(19) United States
(12) Patent Application Publication
(10) Pub. No.: US 2022/0173325 A1
(43) Pub. Date: Jun. 2, 2022

(54) LIGHT EMITTING DIODE AND AMINE COMPOUND FOR THE SAME

(71) Applicant: Samsung Display Co., Ltd., Yongin-si (KR)

(72) Inventors: Taku IMAIZUMI, Yokohama (JP); Takao SAKUMA, Yokohama (JP)

(73) Assignee: Samsung Display Co., Ltd., Yongin-si (KR)

(21) Appl. No.: 17/381,815

(22) Filed: Jul. 21, 2021

(30) Foreign Application Priority Data
Nov. 13, 2020 (KR) 10-2020-0152068

(51) Int. Cl. H01L 51/00

(52) U.S. Cl. CPC H01L 51/0061; H01L 51/0072; H01L 51/0073; H01L 51/5056; H01L 51/0058; H01L 51/006; H01L 51/0074

(57) ABSTRACT

A light emitting diode includes a first electrode, a second electrode disposed on the first electrode, and at least one functional layer disposed between the first electrode and the second electrode. The at least one functional layer includes an amine compound represented by Formula 1, thereby showing high emission efficiency properties and improved life characteristics.

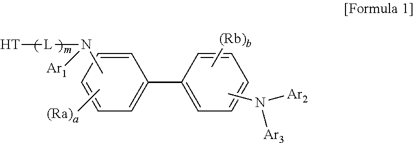

### CROSS-REFERENCE TO RELATED APPLICATION(S)

This application claims priority to and benefits of Korean Patent Application No. 10-2020-0152068 under 35 U.S.C. § 119, filed on Nov. 13, 2020 in the Korean Intellectual Property Office, the entire contents of which are incorporated herein by reference.

### BACKGROUND

### 1. Technical Field

The disclosure relates to an amine compound used in a hole transport region and a light emitting diode including the same.

### 2. Description of the Related Art

Active development continues for an organic electroluminescence display device as an image display device. The organic electroluminescence display device is a so-called self-luminescent display device in which holes and electrons respectively injected from a first electrode and a second electrode recombine in an emission layer so that a light-emitting material in the emission layer emits light to achieve display.

In the application of a light emitting diode to an image display device, there is a need to decrease driving voltage and to increase emission efficiency and device life, and continuous development is required for materials for a light emitting diode which stably achieves such characteristics.

In order to achieve a light emitting diode with high efficiency, development is being conducted on materials for a hole transport region which restrains diffusion of exciton energy in an emission layer.

It is to be understood that this background of the technology section is, in part, intended to provide useful background for understanding the technology. However, this background of the technology section may also include ideas, concepts, or recognitions that were not part of what was known or appreciated by those skilled in the pertinent art prior to a corresponding effective filing date of the subject matter disclosed herein.

### SUMMARY

The disclosure provides a light emitting diode showing excellent emission efficiency and long-life characteristics.

The disclosure also provides an amine compound which is a material for a light emitting diode having high efficiency and long life.

An embodiment provides an amine compound represented by Formula 1 below.

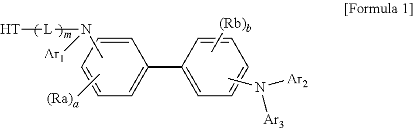

In Formula 1, a and b may each independently be an integer from 0 to 4, Ra and Rb may each independently be a hydrogen atom or a deuterium atom, m may be 0 or 1, L may be a substituted or unsubstituted arylene group of 6 to 40 ring-forming carbon atoms, and HT may be a group represented by Formula 2 below.

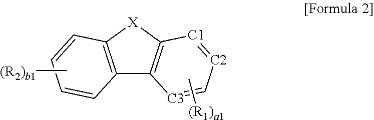

In Formula 2, C1 to C3 each represent a carbon atom in an aromatic ring, any one position among C1 to C3 may be bonded to Formula 1 above. In Formula 2, X may be O or S, a1 may be an integer from 0 to 3, b1 may be an integer from 0 to 4, and R₁ and R₂ may each independently be a hydrogen atom, a deuterium atom, a substituted or unsubstituted alkyl group of 1 to 20 carbon atoms, a substituted or unsubstituted alkenyl group of 2 to 20 carbon atoms, or a substituted or unsubstituted aryl group of 6 to 40 ring-forming carbon atoms, or multiple R₁ groups or multiple R₂ groups may be combined with each other to form an aromatic ring.

In Formula 1, in case where m is 1, Ar₁ to Ar₃ may each independently be a substituted or unsubstituted aryl group of 6 to 40 ring-forming carbon atoms. In case where m is 0, Ar₁ to Ar₃ may each independently be a substituted or unsubstituted aryl group of 6 to 40 ring-forming carbon atoms, or a substituted or unsubstituted heteroaryl group of 5 to 40 ring-forming carbon atoms, or Ar₁ may be a substituted or unsubstituted aryl group of 6 to 40 ring-forming carbon atoms, or a substituted or unsubstituted heteroaryl group of 5 to 40 ring-forming carbon atoms, and Ar₂ and Ar₃ may be combined with an adjacent group to form a carbazole derivative group except for groups represented by CZ-1 to CZ-3 below.

In Formula 1, in case where m is 0, none of Ar₁ to Ar₃ may be a substituted or unsubstituted fluorenyl group, a substituted or unsubstituted pyrenyl group, an unsubstituted carbazolyl group, a 2-dibenzofuranyl group, and a 2-dibenzothiophenyl group, and in case where m is 0, X may not be O, and Ar₁ may not be an unsubstituted phenyl group. In CZ-1 to CZ-3, [symbol] represents a binding site to a neighboring atom.

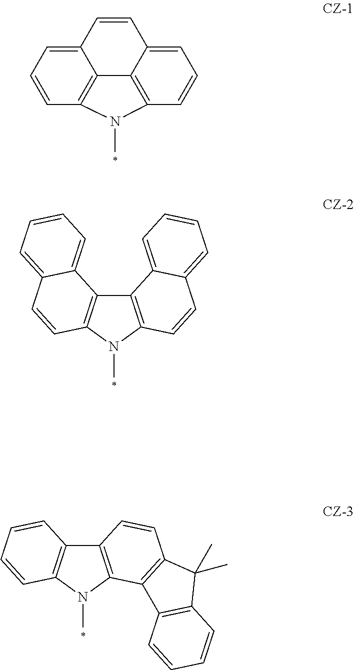 [SMILES: *n1c2cccc3ccc4cccc1c4c32 ; *n1c2ccc3ccccc3c2c2c3ccccc3ccc21 ; *n1c2ccccc2c2ccc3c(c21)-c1ccccc1C3(C)C]

In an embodiment, Formula 1 may be represented by any one among Formula 1-1 to Formula 1-4 below.

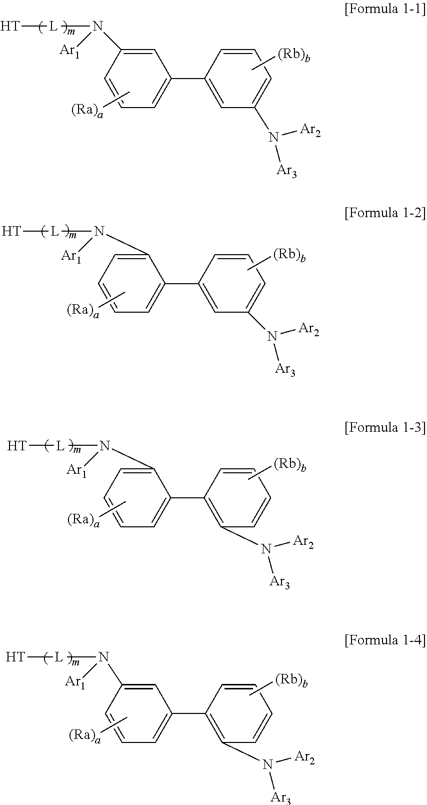

In Formula 1-1 to Formula 1-4, a, b, Ra, Rb, m, L, Ar₁, Ar₂, Ar₃, and HT may be the same as defined in connection with Formula 1 and Formula 2.

In an embodiment, Formula 1 may be represented by Formula 1-A or Formula 1-B below.

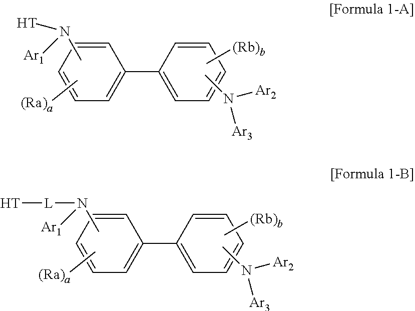

In Formula 1-A and Formula 1-B, a, b, Ra, Rb, L, Ar₁, Ar₂, Ar₃, and HT may be the same as defined in connection with Formula 1 and Formula 2.

In an embodiment, in Formula 1-B, L may be represented by any one among L-1 to L-3 below.

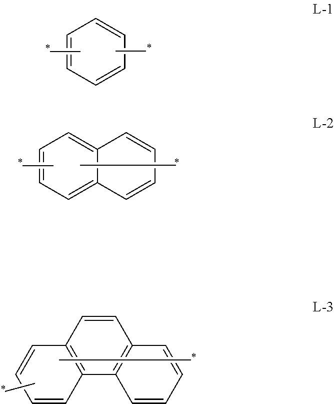

In L-1 to L-3, [symbol] represents a binding site to a neighboring atom.

In an embodiment, Formula 2 may be represented by any one among Formula 2-1 to Formula 2-3 below.

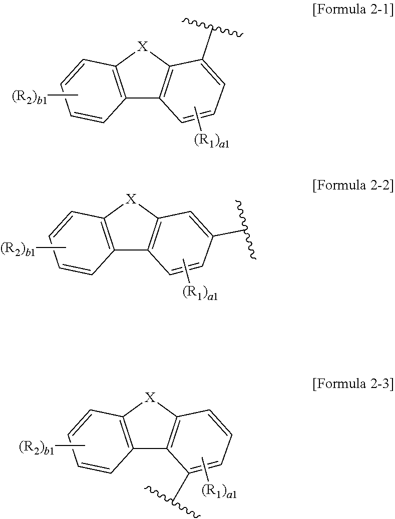

In Formula 2-1 to Formula 2-3, X, a1, b1, R₁, and R₂ may be the same as defined in connection with Formula 2, and

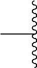

represents a binding site to a neighboring atom.

In an embodiment, Formula 2 may be represented by any one among Formula 2-a to Formula 2-e below.

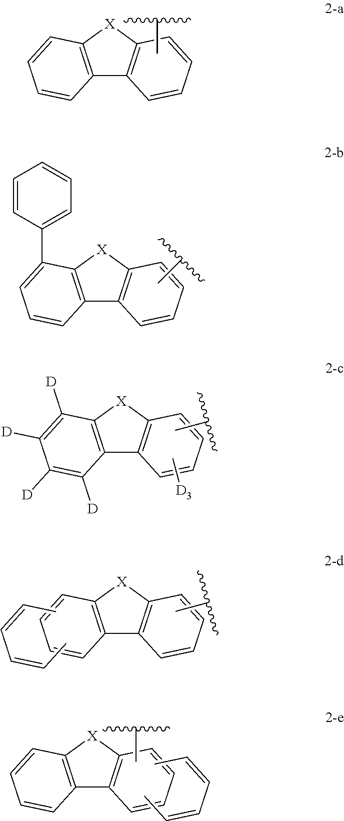

In Formula 2-a to Formula 2-e, X may be the same as defined in connection with Formula 2, and

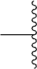

represents a binding site to Formula 1 at any one position among C1 to C3 in Formula 2.

In an embodiment, at least one among Ra, Rb, R₁, and R₂ may be a deuterium atom, or at least one among Ar₁ to Ar₃ may be an aryl group of 6 to 40 ring-forming carbon atoms, which is substituted with at least one deuterium atom.

In an embodiment, a and b may each be 4, and Ra and Rb may each be deuterium atoms.

In an embodiment, in case where m is 0, and at least one among Ar₁ to Ar₃ is a substituted or unsubstituted heteroaryl group of 5 to 40 ring-forming carbon atoms, the heteroaryl group may include O or S as a heteroatom.

Another embodiment provides a light emitting diode which may include a first electrode, a second electrode disposed on the first electrode, and at least one functional layer disposed between the first electrode and the second electrode, the at least one functional layer may include the amine compound of an embodiment.

In an embodiment, the at least one functional layer may include an emission layer, a hole transport region disposed between the first electrode and the emission layer, and an electron transport region disposed between the emission layer and the second electrode, and the hole transport region may include the amine compound.

In an embodiment, the hole transport region may include at least one of a hole injection layer, a hole transport layer, and an electron blocking layer, and at least one among the hole injection layer, the hole transport layer, and the electron blocking layer may include the amine compound.

### BRIEF DESCRIPTION OF THE DRAWINGS

The accompanying drawings are included to provide a further understanding of the embodiments and are incorporated in and constitute a part of this specification. The drawings illustrate embodiments of the disclosure and principles thereof. The above and other aspects and features of the disclosure will become more apparent by describing in detail embodiments thereof with reference to the attached drawings, in which:

FIG. 1 is a plan view showing a display device according to an embodiment;

FIG. 2 is a schematic cross-sectional view of a display device according to an embodiment;

FIG. 3 is a schematic cross-sectional view showing a light emitting diode according to an embodiment;

FIG. 4 is a schematic cross-sectional view showing a light emitting diode according to an embodiment;

FIG. 5 is a schematic cross-sectional view showing a light emitting diode according to an embodiment;

FIG. 6 is a schematic cross-sectional view showing a light emitting diode according to an embodiment;

FIG. 7 is a schematic cross-sectional view of a display device according to an embodiment; and

FIG. 8 is a schematic cross-sectional view of a display device according to an embodiment.

### DETAILED DESCRIPTION OF THE EMBODIMENTS

The disclosure will now be described more fully hereinafter with reference to the accompanying drawings, in which embodiments are shown. This disclosure may, however, be embodied in different forms and should not be construed as limited to the embodiments set forth herein. Rather, these embodiments are provided so that this disclosure will be thorough and complete, and will fully convey the scope of the disclosure to those skilled in the art.

In the drawings, the sizes, thicknesses, ratios, and dimensions of the elements may be exaggerated for ease of description and for clarity. Like numbers refer to like elements throughout.

In the description, it will be understood that when an element (or region, layer, part, etc.) is referred to as being “on”, “connected to”, or “coupled to” another element, it can be directly on, connected to, or coupled to the other element, or one or more intervening elements may be present therebetween. In a similar sense, when an element (or region, layer, part, etc.) is described as “covering” another element, it can directly cover the other element, or one or more intervening elements may be present therebetween.

In the description, when an element is “directly on,” “directly connected to,” or “directly coupled to” another element, there are no intervening elements present. For example, “directly on” may mean that two layers or two elements are disposed without an additional element such as an adhesion element therebetween.

As used herein, the expressions used in the singular such as “a,” “an,” and “the,” are intended to include the plural forms as well, unless the context clearly indicates otherwise.

As used herein, the term “and/or” includes any and all combinations of one or more of the associated listed items. For example, “A and/or B” may be understood to mean “A, B, or A and B.” The terms “and” and “or” may be used in the conjunctive or disjunctive sense and may be understood to be equivalent to “and/or”.

The term “at least one of” is intended to include the meaning of “at least one selected from” for the purpose of its meaning and interpretation. For example, “at least one of A and B” may be understood to mean “A, B, or A and B.” When preceding a list of elements, the term, “at least one of,” modifies the entire list of elements and does not modify the individual elements of the list.

It will be understood that, although the terms first, second, etc. may be used herein to describe various elements, these elements should not be limited by these terms. These terms are only used to distinguish one element from another element. Thus, a first element could be termed a second element without departing from the teachings of the disclosure. Similarly, a second element could be termed a first element, without departing from the scope of the disclosure.

The spatially relative terms “below”, “beneath”, “lower”, “above”, “upper”, or the like, may be used herein for ease of description to describe the relations between one element or component and another element or component as illustrated in the drawings. It will be understood that the spatially relative terms are intended to encompass different orientations of the device in use or operation, in addition to the orientation depicted in the drawings. For example, in the case where a device illustrated in the drawing is turned over, the device positioned “below” or “beneath” another device may be placed “above” another device. Accordingly, the illustrative term “below” may include both the lower and upper positions. The device may also be oriented in other directions and thus the spatially relative terms may be interpreted differently depending on the orientations.

The terms “about” or “approximately” as used herein is inclusive of the stated value and means within an acceptable range of deviation for the recited value as determined by one of ordinary skill in the art, considering the measurement in question and the error associated with measurement of the recited quantity (i.e., the limitations of the measurement system). For example, “about” may mean within one or more standard deviations, or within ±20%, 10%, or 5% of the stated value.

It should be understood that the terms “comprises,” “comprising,” “includes,” “including,” “have,” “having,” “contains,” “containing,” and the like are intended to specify the presence of stated features, integers, steps, operations, elements, components, or combinations thereof in the disclosure, but do not preclude the presence or addition of one or more other features, integers, steps, operations, elements, components, or combinations thereof.

Unless otherwise defined or implied herein, all terms (including technical and scientific terms) used have the same meaning as commonly understood by those skilled in the art to which this disclosure pertains. It will be further understood that terms, such as those defined in commonly used dictionaries, should be interpreted as having a meaning that is consistent with their meaning in the context of the relevant art and should not be interpreted in an ideal or excessively formal sense unless clearly defined in the specification.

In the description, the term “substituted or unsubstituted” corresponds to substituted or unsubstituted with at least one substituent selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. Each of the substituents may be substituted or unsubstituted. For example, a biphenyl group may be interpreted as an aryl group or a phenyl group substituted with a phenyl group.

In the description, the term “forming a ring via the combination with an adjacent group” may mean forming a substituted or unsubstituted hydrocarbon ring, or a substituted or unsubstituted heterocycle via the combination with an adjacent group. The hydrocarbon ring may include an aliphatic hydrocarbon ring and an aromatic hydrocarbon ring. The heterocycle may include an aliphatic heterocycle and an aromatic heterocycle. The hydrocarbon ring and the heterocycle may each be a monocyclic ring or a polycyclic ring. A ring formed via combination with an adjacent group may be combined with another ring to form a spiro structure.

In the description, the term “adjacent group” may mean a substituent substituted for an atom which is directly combined with an atom substituted with a corresponding substituent, another substituent substituted for an atom which is substituted with a corresponding substituent, or a substituent sterically positioned at the nearest position to a corresponding substituent. For example, in 1,2-dimethylbenzene, two methyl groups may be interpreted as “adjacent groups” to each other, and in 1,1-diethylcyclopentene, two ethyl groups may be interpreted as “adjacent groups” to each other.

In the description, examples of the halogen atom may include a fluorine atom, a chlorine atom, a bromine atom, and an iodine atom.

In the description, the alkyl group may be a linear, a branched, or a cyclic type. The number of carbon atoms in the alkyl group may be 1 to 50, 1 to 30, 1 to 20, 1 to 10, or 1 to 6. Examples of the alkyl group may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-heneicosyl, n-docosyl, n-tricosyl, n-tetracosyl, n-pentacosyl, n-hexacosyl, n-heptacosyl, n-octacosyl, n-nonacosyl, n-triacontyl, etc., without limitation.

In the description, the hydrocarbon ring group may be an optional functional group or substituent derived from an aliphatic hydrocarbon ring group. The hydrocarbon ring group may be a saturated hydrocarbon ring group of 5 to 20 ring-forming carbon atoms.

In the description, the aryl group may be an optional functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include phenyl, naphthyl, fluorenyl, anthracenyl, phenanthryl, biphenyl, terphenyl, quaterphenyl, quinquephenyl, sexiphenyl, triphenylenyl, pyrenyl, benzofluoranthenyl, chrysenyl, etc., without limitation.

In the description, the fluorenyl group may be substituted, and two substituents may be combined with each other to form a spiro structure. Examples of a substituted fluorenyl group are as follows but embodiments are not limited thereto.

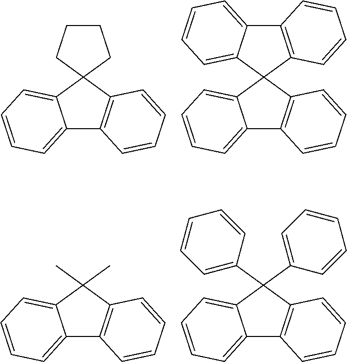

In the description, the heterocyclic group may be an optional functional group or substituent derived from a ring including at least one of B, O, N, P, Si, and S as heteroatoms. The heterocyclic group may include an aliphatic heterocyclic group and an aromatic heterocyclic group. The aromatic heterocyclic group may be a heteroaryl group. The aliphatic heterocyclic group and the aromatic heterocyclic group may be a monocycle or a polycycle.

In the description, the heterocyclic group may include at least one of B, O, N, P, Si, and S as heteroatoms. If the heterocyclic group includes two or more heteroatoms, two or more heteroatoms may be the same or different. The heterocyclic group may be a monocyclic heterocyclic group or a polycyclic heterocyclic group, and may include a heteroaryl group. The number ring-forming carbon atoms in the heteroaryl group may be 2 to 30, 2 to 20, 2 to 12, or 2 to 10.

In the description, the aliphatic heterocyclic group may include at least one of B, O, N, P, Si, and S as heteroatoms. The number of ring-forming carbon atoms in the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., without limitation.

In the description, the heteroaryl group may include at least one of B, O, N, P, Si, and S as heteroatoms. If the heteroaryl group includes two or more heteroatoms, two or more heteroatoms may be the same or different. The heteroaryl group may be a monocyclic heterocyclic group or polycyclic heterocyclic group. The number of ring-forming carbon atoms in the heteroaryl group may be 2 to 30, 2 to 20, or 2 to 10. Examples of the heteroaryl group may include thiophene, furan, pyrrole, imidazole, triazole, pyridine, bipyridine, pyrimidine, triazine, triazole, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, carbazole, N-arylcarbazole, N-heteroarylcarbazole, N-alkylcarbazole, benzoxazole, benzoimidazole, benzothiazole, benzocarbazole, benzothiophene, dibenzothiophene, thienothiophene, benzofurane, phenanthroline, thiazole, isooxazole, oxazole, oxadiazole, thiadiazole, phenothiazine, dibenzosilole, dibenzofuran, etc., without limitation.

In the description, the explanation on the aryl group may be applied to an arylene group except that the arylene group is a divalent group. The explanation on the heteroaryl group may be applied to a heteroarylene group except that the heteroarylene group is a divalent group.

In the description, the silyl group may include an alkyl silyl group and an aryl silyl group. Examples of the silyl group may include a trimethylsilyl group, a triethylsilyl group, a t-butyldimethylsilyl group, a propyldimethylsilyl group, a triphenylsilyl group, a diphenylsilyl group, a phenylsilyl group, etc., without limitation.

In the description, the number of carbon atoms in the amino group is not specifically limited, but may be 1 to 30. The amino group may include an alkyl amino group, an aryl amino group, or a heteroaryl amino group. Examples of the amino group may include a methylamino group, a dimethylamino group, a phenylamino group, a diphenylamino group, a naphthylamino group, a 9-methyl-anthracenylamino group, a triphenylamino group, etc., without limitation.

In the description, the number of carbon atoms in a carbonyl group is not specifically limited, but may be 1 to 40, 1 to 30, or 1 to 20. For example, the carbonyl group may have the structures below, but is not limited thereto.

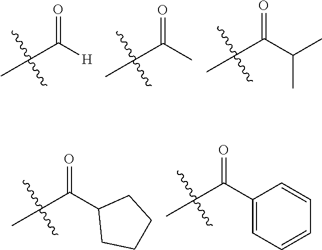

In the description, the number of carbon atoms in the sulfinyl group and sulfonyl group is not specifically limited, but may be 1 to 30. The sulfinyl group may include an alkyl sulfinyl group and an aryl sulfinyl group. The sulfonyl group may include an alkyl sulfonyl group and an aryl sulfonyl group.

In the description, the thio group may include an alkyl thio group and an aryl thio group. The thio group may include the above-defined alkyl group or aryl group combined with a sulfur atom. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, etc., without limitation.

In the description, the oxy group may include the above-defined alkyl group or aryl group which is combined with an oxygen atom. The oxy group may include an alkoxy group and an aryl oxy group. The alkoxy group may be a linear, branched, or cyclic chain. The number of carbon atoms in the alkoxy group is not specifically limited but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, etc. However, embodiments are not limited thereto.

In the description, the boron group may include the above-defined alkyl group or aryl group which is combined with a boron atom. The boron group may include an alkyl boron group and an aryl boron group. Examples of the boron group include a trimethylboron group, a triethylboron group, a t-butyldimethylboron group, a triphenylboron group, a diphenylboron group, a phenylboron group, etc., without limitation.

In the description, the alkenyl group may be a linear chain or a branched chain. The number of carbon atoms in the alkenyl group is not specifically limited but may be 2 to 30, 2 to 20, or 2 to 10. Examples of the alkenyl group may include a vinyl group, a 1-butenyl group, a 1-pentenyl group, a 1,3-butadienyl aryl group, a styrenyl group, a styrylvinyl group, etc., without limitation.

In the description, the number of carbon atoms in the amine group is not specifically limited, but may be 1 to 30. The amine group may include an alkyl amine group and an aryl amine group. Examples of the amine group include a methylamine group, a dimethylamine group, a phenylamine group, a diphenylamine group, a naphthylamine group, a 9-methyl-anthracenylamine group, a triphenylamine group, etc., without limitation.

In the description, an alkyl group in the alkylthio group, alkylsulfoxy group, alkylaryl group, alkylamino group, alkylboron group, alkyl silyl group, and alkyl amine group may be the same as the examples of the above-described alkyl group.

In the description, the aryl group in the aryloxy group, arylthio group, arylsulfoxy group, aryl amino group, arylboron group, and aryl silyl group may be the same as the examples of the above-described aryl group.

In the description, a direct linkage may be a single bond.

In the description,

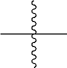

and [symbol] each indicate a binding site to a neighboring atom.

Hereinafter, embodiments will be explained referring to the drawings.

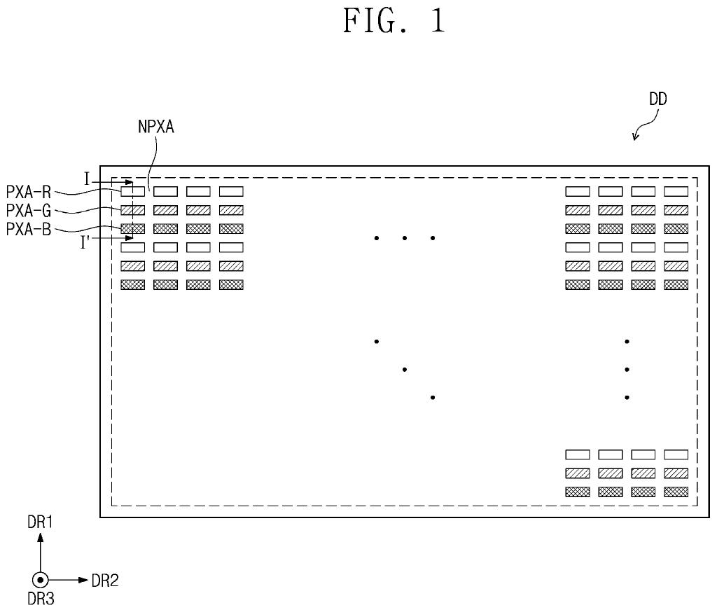

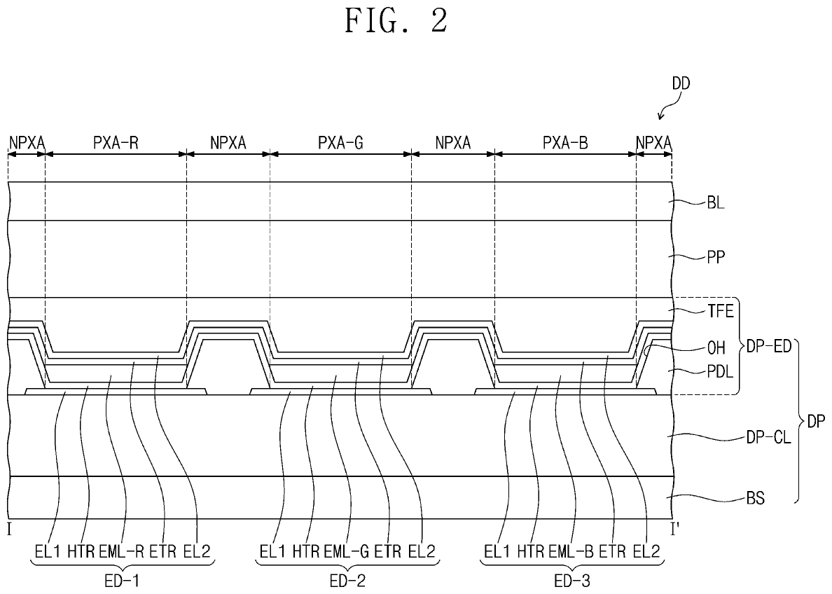

FIG. 1 is a plan view showing an embodiment of a display device DD. FIG. 2 is a schematic cross-sectional view of a display device DD of an embodiment. FIG. 2 is a schematic cross-sectional view showing a part corresponding to line I-I′.

The display device DD may include a display panel DP and an optical layer PP disposed on the display panel DP. The display panel DP includes light emitting diodes ED-1, ED-2, and ED-3. The display device DD may include multiple light emitting diodes ED-1, ED-2, and ED-3. The optical layer PP may be disposed on the display panel DP and may control light reflected from an external light at the display panel DP. The optical layer PP may include, for example, a polarization layer or a color filter layer. Although not shown in the drawings, in an embodiment, the optical layer PP may be omitted in the display device DD.

A base substrate BL may be disposed on the optical layer PP. The base substrate BL may be a member providing a base surface where the optical layer PP is disposed. The base substrate BL may be a glass substrate, a metal substrate, a plastic substrate, etc. However, embodiments are not limited thereto, and the base substrate BL may include an inorganic layer, an organic layer, or a composite material layer. Although not shown in the drawings, the base substrate BL may be omitted in an embodiment.

The display device DD according to an embodiment may further include a filling layer (not shown). The filling layer (not shown) may be disposed between a display device layer DP-ED and a base substrate BL. The filling layer (not shown) may include an organic layer. The filling layer (not shown) may include at least one of an acrylic resin, a silicon-based resin, and an epoxy-based resin.

The display panel DP may include a base layer BS, a circuit layer DP-CL provided on the base layer BS, and a display element layer DP-ED. The display element layer DP-ED may include a pixel definition layer PDL, light emitting diodes ED-1, ED-2, and ED-3 disposed in the pixel definition layer PDL, and an encapsulating layer TFE disposed on the light emitting diodes ED-1, ED-2, and ED-3.

The base layer BS may be a member providing a base surface where the display device element layer DP-ED is disposed. The base layer BS may be a glass substrate, a metal substrate, a plastic substrate, etc. However, embodiments are not limited thereto, and the base layer BS may include an inorganic layer, an organic layer, or a composite material layer.

In an embodiment, the circuit layer DP-CL is disposed on the base layer BS, and the circuit layer DP-CL may include multiple transistors (not shown). Each of the transistors (not shown) may include a control electrode, an input electrode, and an output electrode. For example, the circuit layer DP-CL may include switching transistors and driving transistors for driving the light emitting diodes ED-1, ED-2, and ED-3 of the display element layer DP-ED.

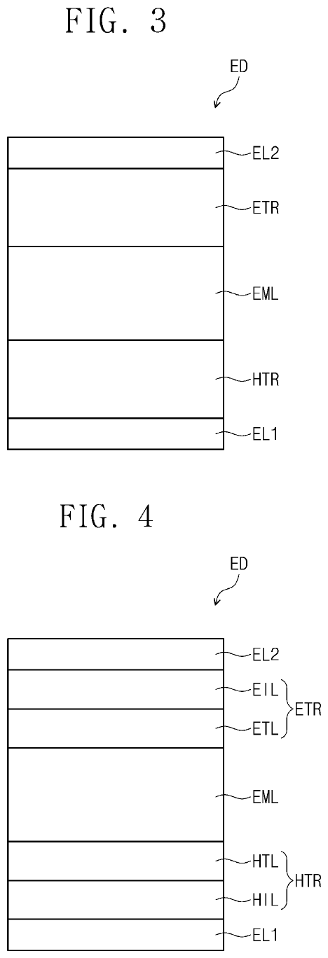

Each of the light emitting diodes ED-1, ED-2, and ED-3 may have the structures of light emitting diodes ED of embodiments according to FIG. 3 to FIG. 6, which will be explained later. Each of the light emitting diodes ED-1, ED-2, and ED-3 may include a first electrode EL1, a hole transport region HTR, emission layers EML-R, EML-G, and EML-B, an electron transport region ETR, and a second electrode EL2.

In FIG. 2, shown is an embodiment where the emission layers EML-R, EML-G, and EML-B of light emitting diodes ED-1, ED-2, and ED-3, are disposed in opening portions OH defined in a pixel definition layer PDL, and a hole transport region HTR, an electron transport region ETR, and a second electrode EL2 are provided as common layers in all light emitting diodes ED-1, ED-2, and ED-3. However, embodiments are not limited thereto. Although not shown in FIG. 2, in an embodiment, the hole transport region HTR and the electron transport region ETR may be patterned and provided in the opening portions OH defined in the pixel definition layer PDL. For example, in an embodiment, the hole transport region HTR, the emission layers EML-R, EML-G, and EML-B, and the electron transport region ETR of the light emitting diodes ED-1, ED-2, and ED-3 may be patterned by an ink jet printing method and provided.

An encapsulating layer TFE may cover the light emitting diodes ED-1, ED-2, and ED-3. The encapsulating layer TFE may encapsulate the display element layer DP-ED. The encapsulating layer TFE may be a thin film encapsulating layer. The encapsulating layer TFE may be one layer or a stack layer of layers. The encapsulating layer TFE may include at least one insulating layer. The encapsulating layer TFE according to an embodiment may include at least one inorganic layer (hereinafter, encapsulating inorganic layer). The encapsulating layer TFE according to an embodiment may include at least one organic layer (hereinafter, encapsulating organic layer) and at least one encapsulating inorganic layer.

The encapsulating inorganic layer may protect the display element layer DP-ED from moisture and/or oxygen, and the encapsulating organic layer may protect the display element layer DP-ED from foreign materials such as dust particles. The encapsulating inorganic layer may include silicon nitride, silicon oxy nitride, silicon oxide, titanium oxide, or aluminum oxide, without specific limitation. The encapsulating organic layer may include an acrylic compound, an epoxy-based compound, etc. The encapsulating organic layer may include a photopolymerizable organic material, without specific limitation.

The encapsulating layer TFE may be disposed on the second electrode EL2 and may be disposed to fill the opening portion OH.

Referring to FIG. 1 and FIG. 2, the display device DD may include a non-emitting area NPXA and emitting areas PXA-R, PXA-G, and PXA-B. The emitting areas PXA-R, PXA-G, and PXA-B may be areas emitting light produced from the light emitting diodes ED-1, ED-2, and ED-3, respectively. The emitting areas PXA-R, PXA-G, and PXA-B may be separated from each other on a plane.

The emitting areas PXA-R, PXA-G, and PXA-B may be areas separated by the pixel definition layer PDL. The non-emitting areas NPXA may be areas between neighboring emitting areas PXA-R, PXA-G, and PXA-B and may be areas corresponding to the pixel definition layer PDL. In the disclosure, each of the emitting areas PXA-R, PXA-G, and PXA-B may correspond to a pixel. The pixel definition layer PDL may divide the light emitting diodes ED-1, ED-2, and ED-3. The emission layers EML-R, EML-G, and EML-B of the light emitting diodes ED-1, ED-2, and ED-3 may be disposed and divided in the opening portions OH defined in the pixel definition layer PDL.

The emitting areas PXA-R, PXA-G, and PXA-B may be divided into groups according to the color of light produced from the light emitting diodes ED-1, ED-2, and ED-3. In the display device DD of an embodiment, shown in FIG. 1 and FIG. 2, three emitting areas PXA-R, PXA-G, and PXA-B respectively emitting red light, green light, and blue light are illustrated as an embodiment. For example, the display device DD of an embodiment may include a red emitting area PXA-R, a green emitting area PXA-G, and a blue emitting area PXA-B, which are separated from each other.

In the display device DD according to an embodiment, multiple light emitting diodes ED-1, ED-2, and ED-3 may emit light having different wavelength regions. For example, in an embodiment, the display device DD may include a first light emitting diode ED-1 emitting red light, a second light emitting diode ED-2 emitting green light, and a third light emitting diode ED-3 emitting blue light. For example, each of the red emitting area PXA-R, the green emitting area PXA-G, and the blue emitting area PXA-B of the display device DD may correspond to the first light emitting diode ED-1, the second light emitting diode ED-2, and the third light emitting diode ED-3.

However, embodiments are not limited thereto, and the first to third light emitting diodes ED-1, ED-2, and ED-3 may emit light in a same wavelength region, or at least one thereof may emit light in a different wavelength region. For example, all of the first to third light emitting diodes ED-1, ED-2, and ED-3 may emit blue light.

The emitting areas PXA-R, PXA-G, and PXA-B in the display device DD according to an embodiment may be arranged in a stripe shape. Referring to FIG. 1, multiple red emitting areas PXA-R, multiple green emitting areas PXA-G, and multiple blue emitting areas PXA-B may be arranged along a second directional axis DR2. The red emitting area PXA-R, the green emitting area PXA-G, and the blue emitting area PXA-B may be arranged by turns along a first directional axis DR1.

In FIG. 1 and FIG. 2, the areas of the emitting areas PXA-R, PXA-G, and PXA-B are shown as having a similar size, but embodiments are not limited thereto. The areas of the emitting areas PXA-R, PXA-G, and PXA-B may be different from each other according to the wavelength region of light emitted. The areas of the emitting areas PXA-R, PXA-G, and PXA-B may be areas in a plan view that are defined by the first directional axis DR1 and the second directional axis DR2.

The arrangement of the emitting areas PXA-R, PXA-G, and PXA-B is not limited to the configuration shown in FIG. 1, and the arrangement of the red emitting areas PXA-R, the green emitting areas PXA-G, and the blue emitting areas PXA-B may be provided in various combinations according to the properties of display quality required for the display device DD. For example, the arrangement type of the emitting areas PXA-R, PXA-G, and PXA-B may be a PenTile® arrangement type, or a diamond arrangement type.

The areas of the emitting areas PXA-R, PXA-G and PXA-B may be different from each other. For example, in an embodiment, the area of the green emitting area PXA-G may be smaller than the area of the blue emitting area PXA-B, but embodiments are not limited thereto.

Hereinafter, FIG. 3 to FIG. 6 are schematic cross-sectional views showing light emitting diodes according to embodiments. The light emitting diode ED according to an embodiment may include a first electrode EL1, a second electrode EL2 facing and disposed on the first electrode EL1, and at least one functional layer disposed between the first electrode EL1 and the second electrode EL2. The at least one functional layer may include a hole transport region HTR, an emission layer EML, and an electron transport region ETR, stacked in order. For example, the light emitting diode ED of an embodiment may include a first electrode EL1, a hole transport region HTR, an emission layer EML, an electron transport region ETR, and a second electrode EL2, stacked in that order.

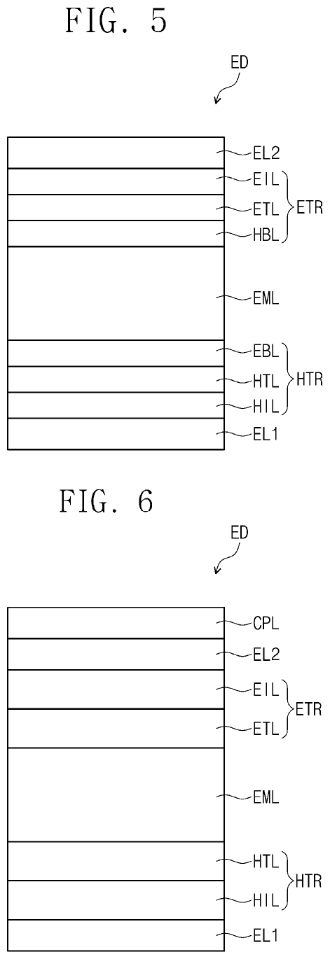

In comparison to FIG. 3, FIG. 4 shows a schematic cross-sectional view of a light emitting diode ED of an embodiment, wherein a hole transport region HTR includes a hole injection layer HIL and a hole transport layer HTL, and an electron transport region ETR includes an electron injection layer EIL and an electron transport layer ETL. In comparison to FIG. 3, FIG. 5 shows a schematic cross-sectional view of a light emitting diode ED of an embodiment, wherein a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. In comparison to FIG. 4, FIG. 6 shows a schematic cross-sectional view of a light emitting diode ED of an embodiment, including a capping layer CPL disposed on the second electrode EL2.

The light emitting diode ED of an embodiment may include an amine compound of an embodiment, which will be explained later, in at least one functional layer, such as a hole transport region HTR, an emission layer EML, and an electron transport region ETR.

In the light emitting diode ED according to an embodiment, the first electrode EL1 has conductivity. The first electrode EL1 may be formed using a metal material, a metal alloy, or a conductive compound. The first electrode EL1 may be an anode or a cathode. However, embodiments are not limited thereto. In an embodiment, the first electrode EL1 may be a pixel electrode. The first electrode EL1 may be a transmissive electrode, a transflective electrode, or a reflective electrode. If the first electrode EL1 is a transmissive electrode, the first electrode EL1 may include a transparent metal oxide such as indium tin oxide (ITO), indium zinc oxide (IZO), zinc oxide (ZnO), and indium tin zinc oxide (ITZO). If the first electrode EL1 is a transflective electrode or a reflective electrode, the first electrode EL1 may include Ag, Mg, Cu, Al, Pt, Pd, Au, Ni, Nd, Ir, Cr, Li, Ca, LiF/Ca, LiF/Al, Mo, Ti, W, compounds thereof, or mixtures thereof (for example, a mixture of Ag and Mg). In an embodiment, the first electrode EL1 may have a structure including multiple layers including a reflective layer or a transflective layer formed using the above materials, and a transmissive conductive layer formed using ITO, IZO, ZnO, or ITZO. For example, the first electrode EL1 may include a three-layer structure of ITO/Ag/ITO. However, embodiments are not limited thereto. The first electrode EL1 may include the above-described metal materials, combinations of two or more metal materials selected from the above-described metal materials, or oxides of the above-described metal materials, without limitation. A thickness of the first electrode EL1 may be in a range of about 700 Å to about 10,000 Å. For example, the thickness of the first electrode EL1 may be in a range of about 1,000 Å to about 3,000 Å.

The hole transport region HTR may be provided on the first electrode EL1. The hole transport region HTR may include at least one of a hole injection layer HIL, a hole transport layer HTL, a buffer layer (not shown), an emission auxiliary layer (not shown), and an electron blocking layer EBL. A thickness of the hole transport region HTR may be in a range of about 50 Å to about 15,000 Å.

The hole transport region HTR may have a single layer formed using a single material, a single layer formed using different materials, or a multilayer structure including layers formed using different materials.

For example, the hole transport region HTR may have the structure of a single layer of a hole injection layer HIL or a hole transport layer HTL, and may have a structure of a single layer formed using a hole injection material and a hole transport material. In another embodiment, the hole transport region HTR may have a structure of a single layer formed using multiple different materials, or a structure stacked from the first electrode EL1 of hole injection layer HIL/hole transport layer HTL, hole injection layer HIL/hole transport layer HTL/buffer layer (not shown), hole injection layer HIL/buffer layer (not shown), hole transport layer HTL/buffer layer (not shown), or hole injection layer HIL/hole transport layer HTL/electron blocking layer EBL, without limitation.

The hole transport region HTR may be formed using various methods such as a vacuum deposition method, a spin coating method, a cast method, a Langmuir-Blodgett (LB) method, an inkjet printing method, a laser printing method, and a laser induced thermal imaging (LITI) method.

In the light emitting diode ED of an embodiment, the hole transport region HTR may include an amine compound represented by Formula 1 below. In the light emitting diode ED of an embodiment, a hole transport region HTR may include at least one of a hole injection layer HIL, a hole transport layer HTL, and an electron blocking layer EBL, and at least one among the hole injection layer HIL, the hole transport layer HTL, and the electron blocking layer EBL may include an amine compound of an embodiment, represented by Formula 1 below. For example, in the light emitting diode ED of an embodiment, the hole transport layer HTL may include an amine compound represented by Formula 1 below.

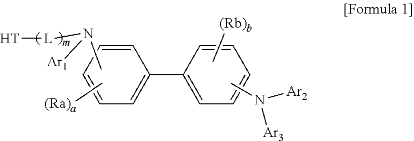

In Formula 1, HT corresponds to a dibenzoheterole moiety represented by Formula 2 which will be explained later. Thus, amine compound of an embodiment, represented by Formula 1 has a structure in which a biphenyl group may be used as a linking moiety, and each benzene ring of the biphenyl group may be combined with a nitrogen atom of an amine group. In Formula 1, at least one of the amine groups may have a structure including a dibenzoheterole ring.

In Formula 1, a and b may each independently be an integer from 0 to 4, and Ra and Rb may be each independently a hydrogen atom or a deuterium atom. For example, in the amine compound of an embodiment, represented by Formula 1, Ra and Rb may each be hydrogen atoms, or at least one among multiple Ra groups and multiple Rb groups may be a deuterium atom. In an embodiment, in case where a and b are each 4, all of four Ra groups and four Rb groups may each be deuterium atoms.

In Formula 1, m may be 0 or 1. In case where m is 0, a dibenzoheterole group represented by HT may be directly bonded to a nitrogen atom. For example, in case where m is 0, a dibenzoheterole group represented by HT may be bonded to a nitrogen atom of an amine group via a single bond. In case where m is 1, a dibenzoheterole group represented by HT may be bonded to a nitrogen atom of an amine group via a linking moiety represented by L.

In Formula 1, in case where m is 1, Ar₁ to Ar₃ may each independently be a substituted or unsubstituted aryl group of 6 to 40 ring-forming carbon atoms. In Formula 1, in case where m is 0, Ar₁ to Ar₃ may each independently be a substituted or unsubstituted aryl group of 6 to 40 ring-forming carbon atoms, or a substituted or unsubstituted heteroaryl group of 5 to 40 ring-forming carbon atoms; or Ar₁ may be a substituted or unsubstituted aryl group of 6 to 40 ring-forming carbon atoms, or a substituted or unsubstituted heteroaryl group of 5 to 40 ring-forming carbon atoms, and Ar₂ and Ar₃ may be combined with an adjacent group to form a carbazole derivative group except for groups represented by CZ-1 to CZ-3 below.

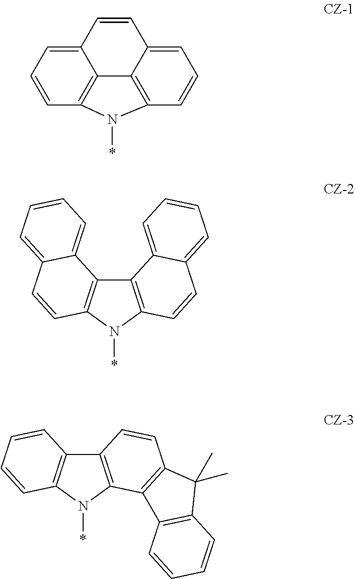 [SMILES: *n1c2cccc3ccc4cccc1c4c32 ; *n1c2ccc3ccccc3c2c2c3ccccc3ccc21 ; *n1c2ccccc2c2ccc3c(c21)-c1ccccc1C3(C)C]

In the amine compound of an embodiment, represented by Formula 1, in case where m is 0, none of Ar₁ to Ar₃ may be a substituted or unsubstituted fluorenyl group, a substituted or unsubstituted pyrenyl group, an unsubstituted carbazolyl group, a 2-dibenzofuranyl group, and a 2-dibenzothiophenyl group. In Formula 1, in case where m is 0, X may not be O, and Ar₁ may not be an unsubstituted phenyl group.

In Formula 1, L may be a substituted or unsubstituted arylene group of 6 to 40 ring-forming carbon atoms. For example, L may be a substituted or unsubstituted phenylene group, a substituted or unsubstituted naphthalene group, a substituted or unsubstituted phenanthrene group, etc. However, embodiments are not limited thereto.

In an embodiment, in case where m is 1, L may be represented by any one among L-1 to L-3 below. In L-1 to L-3 below, [symbol] represents a binding site to a neighboring atom.

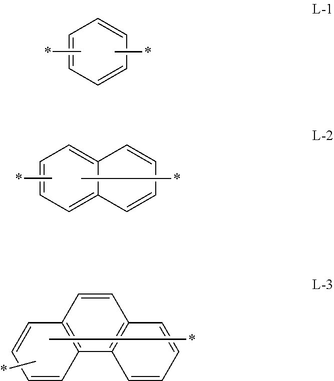

In Formula 1, HT may be represented by Formula 2 below.

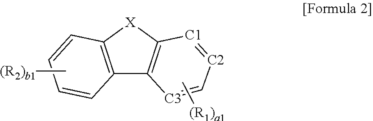

In Formula 2, C1 to C3 each represent a carbon atom in an aromatic ring, and a position selected among C1 to C3 may be a binding site to Formula 1. In Formula 1, in case where m is 0, a nitrogen atom of an amine group in Formula 1 is bonded to Formula 2 at any one position among C1 to C3, and in case where m is 1, any one position among C1 to C3 in Formula 2 is bonded to L in Formula 1.

In Formula 2, X may be O or S. For example, the dibenzoheterole group represented by Formula 2 may be a dibenzofuran derivative, or a dibenzothiophene derivative.

In Formula 2, a1 may be an integer from 0 to 3, and b1 may be an integer from 0 to 4. In case where a1 is 2 or more, multiple R₁ groups may be the same, or at least one thereof may be different from the remainder. In case where b1 is 2 or more, multiple R₂ groups may be the same, or at least one thereof may be different from the remainder.

In Formula 2, R₁ and R₂ may each independently be a hydrogen atom, a deuterium atom, a substituted or unsubstituted alkyl group of 1 to 20 carbon atoms, a substituted or unsubstituted alkenyl group of 2 to 20 carbon atoms, or a substituted or unsubstituted aryl group of 6 to 40 ring-forming carbon atoms, or multiple R₁ groups or multiple R₂ groups may be combined with each other to form an aromatic ring.

In an embodiment, Formula 2 may be represented by any one among Formula 2-1 to Formula 2-3 below.

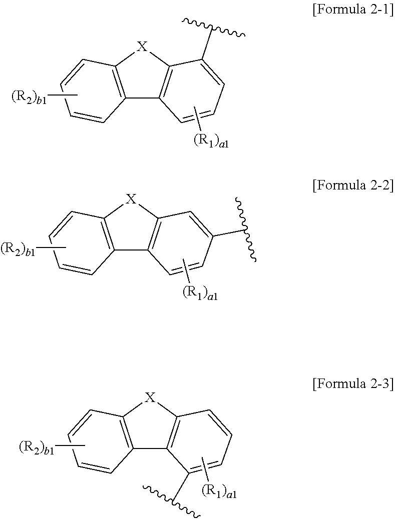

Formula 2-1 corresponds to a case where Formula 1 is bonded at the C1 position of Formula 2, Formula 2-2 corresponds to a case where Formula 1 is bonded at the C2 position of Formula 2, and Formula 2-3 corresponds to a case where Formula 1 is bonded at the C3 position of Formula 2. In Formula 2-1 to Formula 2-3, X, a1, b1, R₁, and R₂ may be the same as defined in connection with Formula 2.

In an embodiment, Formula 2 may be represented by any one among Formula 2-a to Formula 2-e below. In Formula 2,

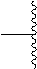

below represents a binding site to Formula 1 at any one position among C1 to C3 in Formula 2. For example, in an embodiment, the dibenzoheterole group represented by Formula 2 may be bonded to Formula 1 at a carbon atom position excluding a carbon atom position corresponding to a para position with respect to a heteroatom represented by X. This is because if the heteroatom of the dibenzoheterole group is combined with the nitrogen atom of an amine group at a para position, an electron excess state may be induced, high reactivity may be attained, and the stability of a compound may be deteriorated.

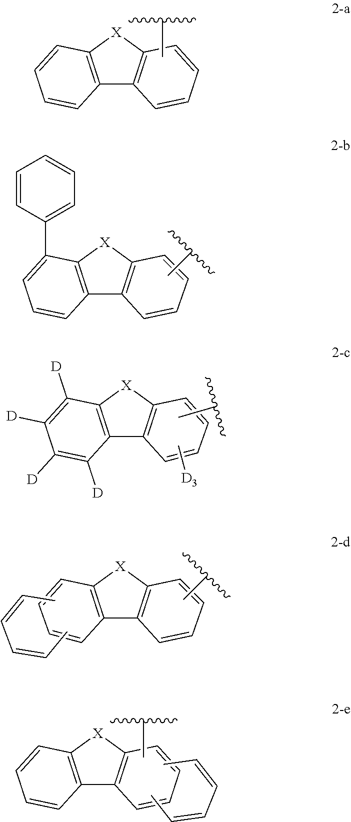

In Formula 2-a to Formula 2-e, X may be the same as defined in connection with Formula 2. Therefore, X in Formula 2-a to Formula 2-e may be O or S. The dibenzoheterole group represented by Formula 2-c corresponds to a case where deuterium atoms are substituted at all carbon atoms excluding a position bonded to Formula 1. The dibenzoheterole group represented by Formula 2-d corresponds to a case where multiple R₂ groups are combined with each other to form an aromatic ring, and the dibenzoheterole group represented by Formula 2-e corresponds to a case where multiple R₁ groups are combined with each other to form an aromatic ring.

In an embodiment, the amine compound represented by Formula 1 may be represented by any one among Formula 1-1 to Formula 1-4 below.

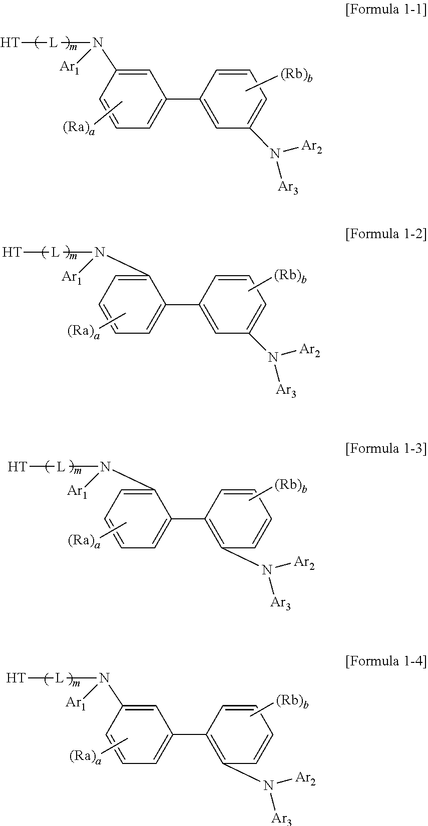

Formula 1-1 corresponds to a case where two amine groups are bonded to different benzene rings of the biphenyl group at meta positions, respectively. Formula 1-3 corresponds to a case where two amine groups are bonded different benzene rings of the biphenyl group at ortho positions, respectively. Formula 1-2 and Formula 1-4 each correspond to cases where one amine group is combined with a benzene ring of the biphenyl group at a meta position, and the other amine group is combined with the other benzene ring of the biphenyl group at an ortho position.

In Formula 1-1 to Formula 1-4, a, b, Ra, Rb, m, L, Ar₁, Ar₂, Ar₃, and HT may be the same as defined in connection with Formula 1 and Formula 2.

In an embodiment, the amine compound may be represented by Formula 1-A or Formula 1-B below.

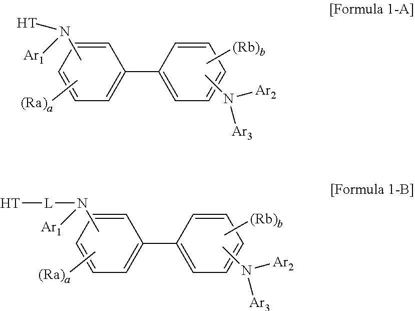

Formula 1-A represents a case where a dibenzoheterole group represented by HT is directly bonded to an amine group that is bonded to the biphenyl group, and Formula 1-B represents a case where a dibenzoheterole group represented by HT is bonded to an amine group that is bonded to the biphenyl group via a linking moiety represented by L.

In Formula 1-A and Formula 1-B, a, b, Ra, Rb, L, Ar₁, Ar₂, Ar₃, and HT may be the same as defined in connection with Formula 1 and Formula 2.

In an embodiment, in the amine compound represented by Formula 1, in case where m is 1, Ar₁ to Ar₃ may each independently be a substituted or unsubstituted aryl group of 6 to 40 ring-forming carbon atoms. For example, if m is 1, Ar₁ to Ar₃ may each independently be a substituted or unsubstituted phenyl group, a substituted or unsubstituted naphthyl group, a substituted or unsubstituted biphenyl group, a substituted or unsubstituted terphenyl group, or a substituted or unsubstituted phenanthrene group. However, embodiments are not limited thereto.

In an embodiment, in the amine compound represented by Formula 1, if m is 0, Ar₁ to Ar₃ may each independently be a substituted or unsubstituted aryl group of 6 to 40 ring-forming carbon atoms, or a substituted or unsubstituted heteroaryl group of 5 to 40 ring-forming carbon atoms, or Ar₁ to Ar₃ may each independently be combined with an adjacent group to form a carbazole derivative group.

In an embodiment, in the amine compound represented by Formula 1, if m is 0, and at least one among Ar₁ to Ar₃ may be a substituted or unsubstituted heteroaryl group of 5 to 40 ring-forming carbon atoms, and the heteroaryl group may include O or S as a heteroatom.

In Formula 1, in case where Ar₂ and Ar₃ are combined with an adjacent group to form a carbazole derivative group, cases of forming carbazole derivative groups represented by CZ-1 to CZ-3 below are excluded.

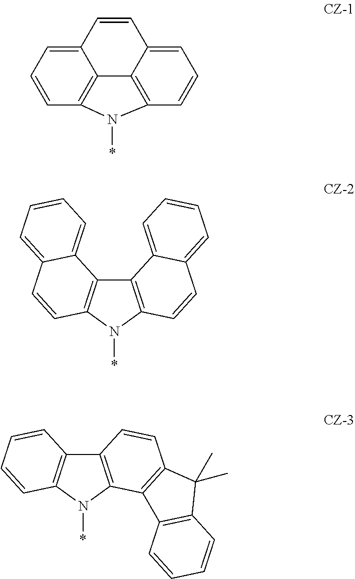 [SMILES: *n1c2cccc3ccc4cccc1c4c32 ; *n1c2ccc3ccccc3c2c2c3ccccc3ccc21 ; *n1c2ccccc2c2ccc3c(c21)-c1ccccc1C3(C)C]

For example, in case where Ar₂ and Ar₃ are combined with an adjacent group to form a carbazole derivative group, the carbazole derivative may be represented by CZ-a or CZ-b below.

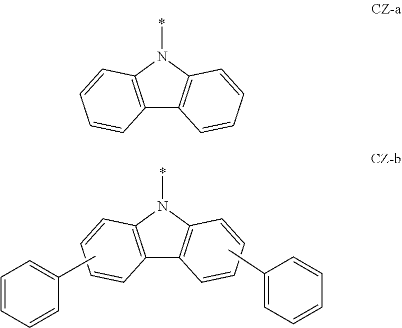

In an embodiment, in the amine compound represented by Formula 1, if m is 0, none of Ar₁ to Ar₃ may be a substituted or unsubstituted fluorenyl group, a substituted or unsubstituted pyrenyl group, an unsubstituted carbazolyl group, a 2-dibenzofuranyl group, and a 2-dibenzothiophenyl group. In an embodiment, in the amine compound represented by Formula 1, if m is 0, X may not be O, and Ar₁ may not be an unsubstituted phenyl group.

In an embodiment, the amine compound represented by Formula 1 may include at least one deuterium substituent. In an embodiment, in Formula 1, at least one among Ra, Rb, R₁, and R₂ may be a deuterium atom, or at least one among Ar₁ to Ar₃ may be an aryl group of 6 to 40 ring-forming carbon atoms substituted with at least one deuterium atom.

In the amine compound of an embodiment represented by Formula 1, the biphenyl moiety may be substituted with at least one deuterium atom. For example, in an embodiment, a and b may each be 4, and Ra and Rb may each be deuterium atoms.

The amine compound of an embodiment, represented by Formula 1, may be represented by any one among the compounds in Compound Group 1A to Compound Group 1Y below. The hole transport region HTR of a light emitting diode ED of an embodiment may include at least one among the amine compounds represented in Compound Group 1A to Compound Group 1Y below.

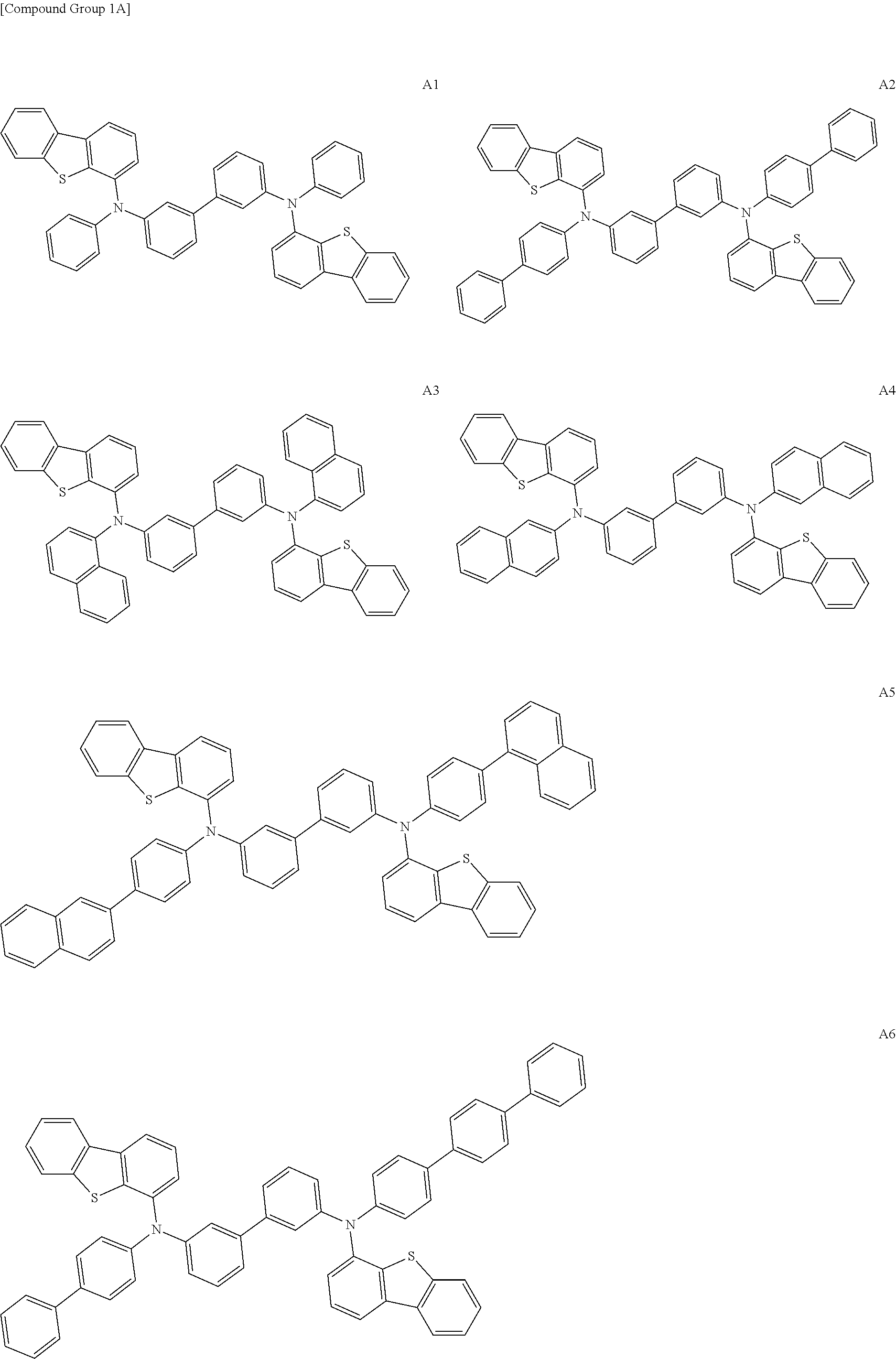

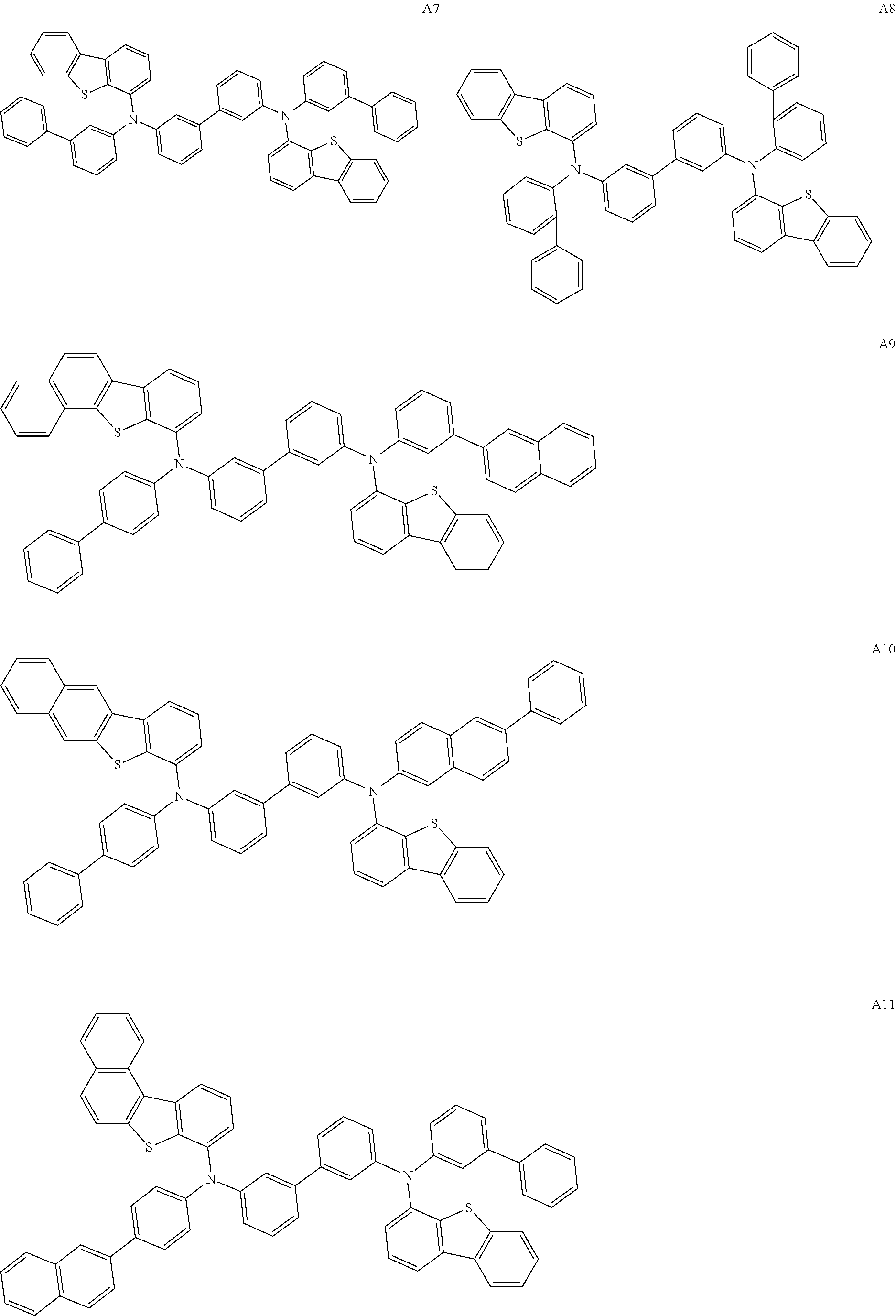

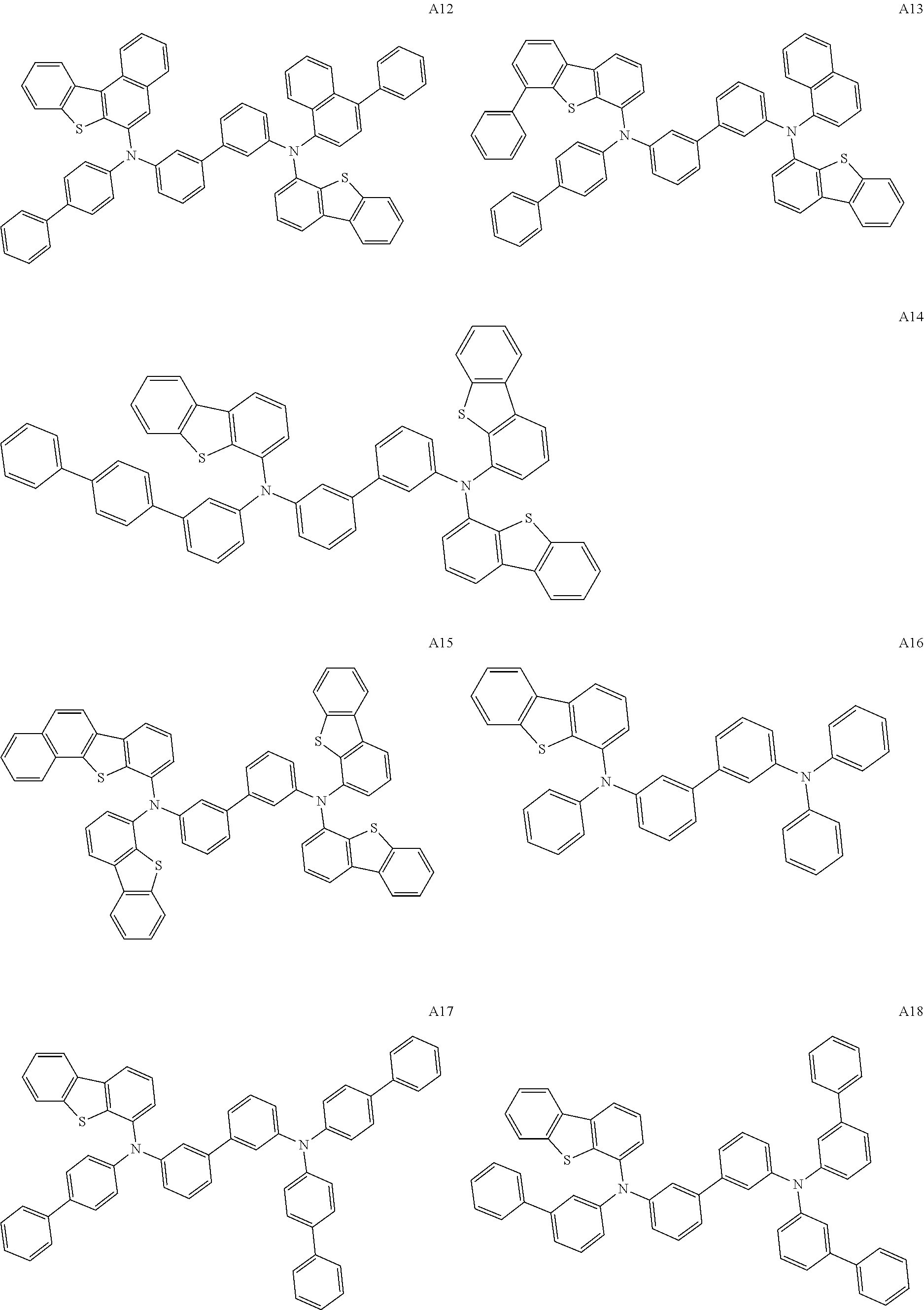

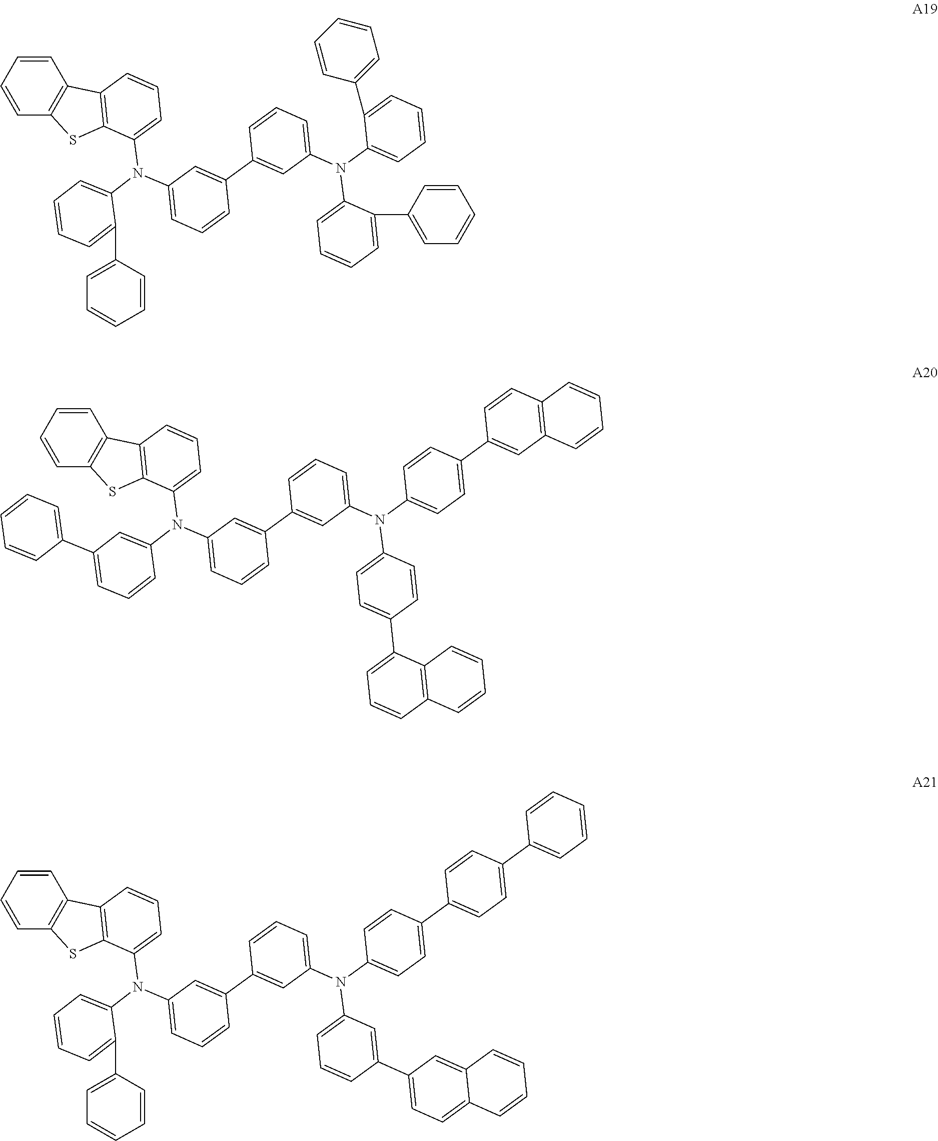 [SMILES: c1ccc(-c2ccccc2N(c2cccc(-c3cccc(N(c4ccccc4-c4ccccc4)c4cccc5c4sc4ccccc45)c3)c2)c2ccccc2-c2ccccc2)cc1 ; c1ccc(-c2cccc(N(c3cccc(-c4cccc(N(c5ccc(-c6ccc7ccccc7c6)cc5)c5ccc(-c6cccc7ccccc67)cc5)c4)c3)c3cccc4c3sc3ccccc34)c2)cc1 ; c1ccc(-c2ccc(-c3ccc(N(c4cccc(-c5cccc(N(c6ccccc6-c6ccccc6)c6cccc7c6sc6ccccc67)c5)c4)c4cccc(-c5ccc6ccccc6c5)c4)cc3)cc2)cc1]

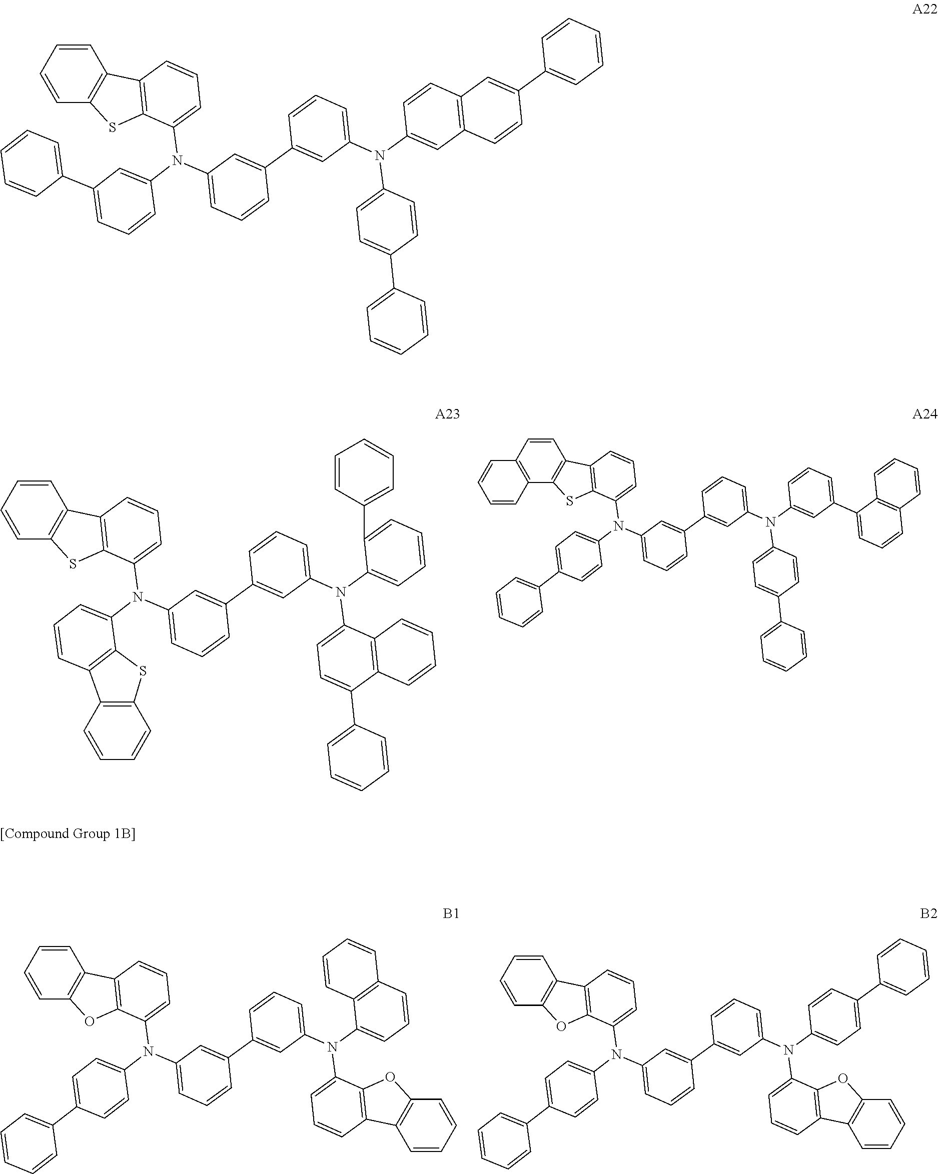

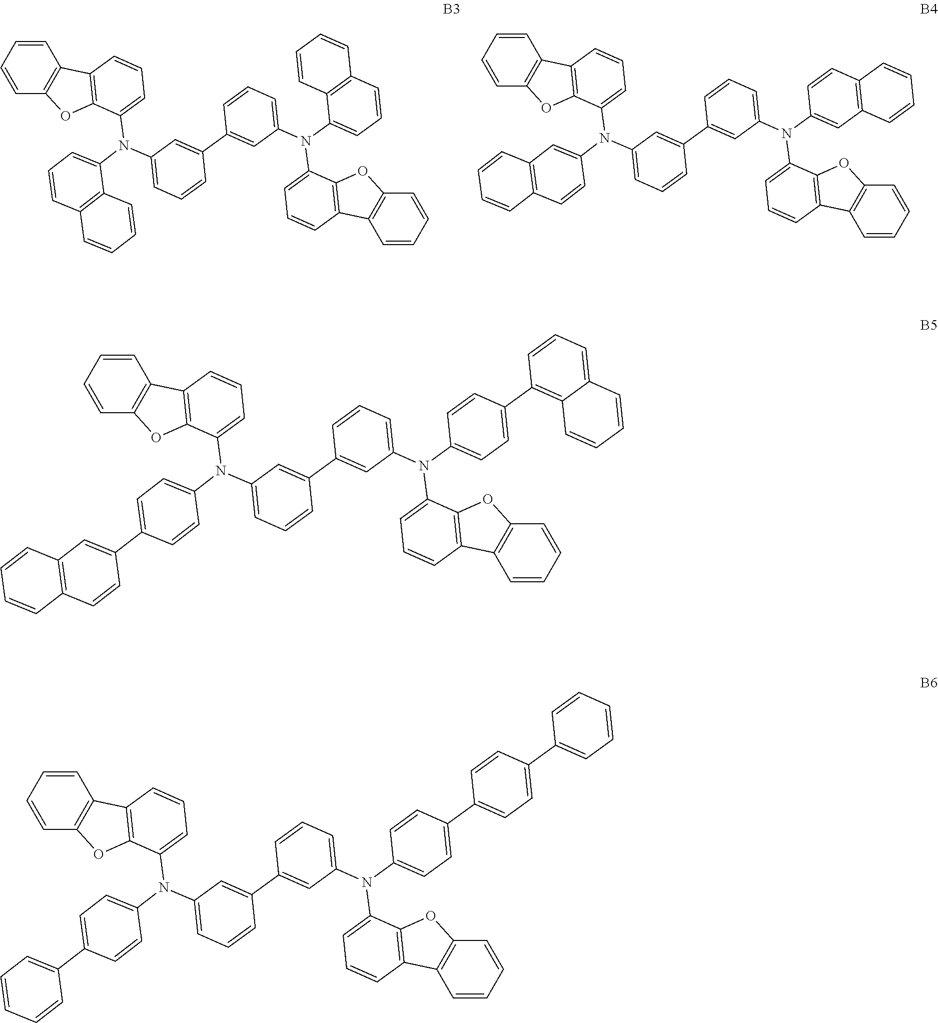 [SMILES: c1cc(-c2cccc(N(c3cccc4ccccc34)c3cccc4c3oc3ccccc34)c2)cc(N(c2cccc3ccccc23)c2cccc3c2oc2ccccc23)c1 ; c1cc(-c2cccc(N(c3ccc4ccccc4c3)c3cccc4c3oc3ccccc34)c2)cc(N(c2ccc3ccccc3c2)c2cccc3c2oc2ccccc23)c1 ; c1cc(-c2cccc(N(c3ccc(-c4cccc5ccccc45)cc3)c3cccc4c3oc3ccccc34)c2)cc(N(c2ccc(-c3ccc4ccccc4c3)cc2)c2cccc3c2oc2ccccc23)c1 ; c1ccc(-c2ccc(-c3ccc(N(c4cccc(-c5cccc(N(c6ccc(-c7ccccc7)cc6)c6cccc7c6oc6ccccc67)c5)c4)c4cccc5c4oc4ccccc45)cc3)cc2)cc1]

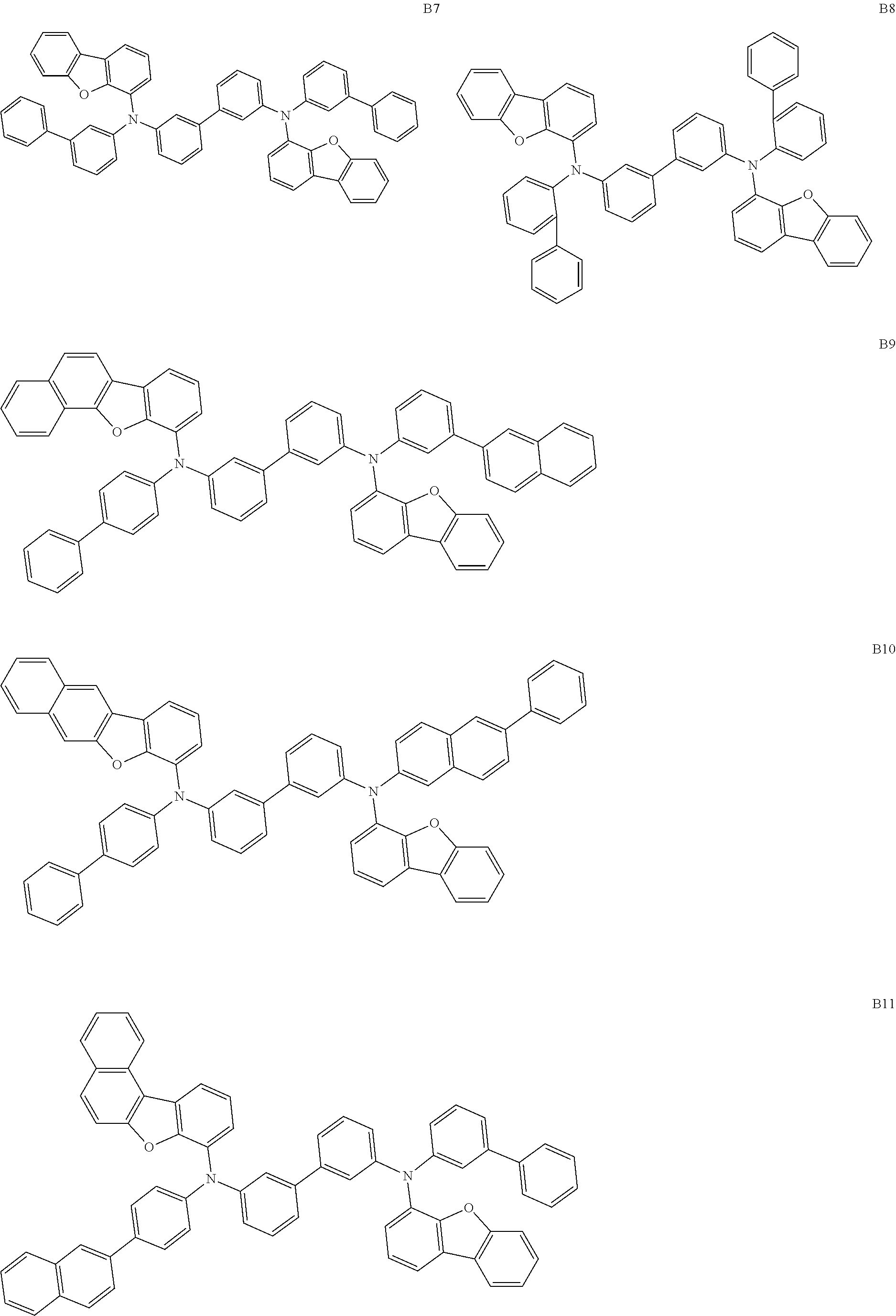

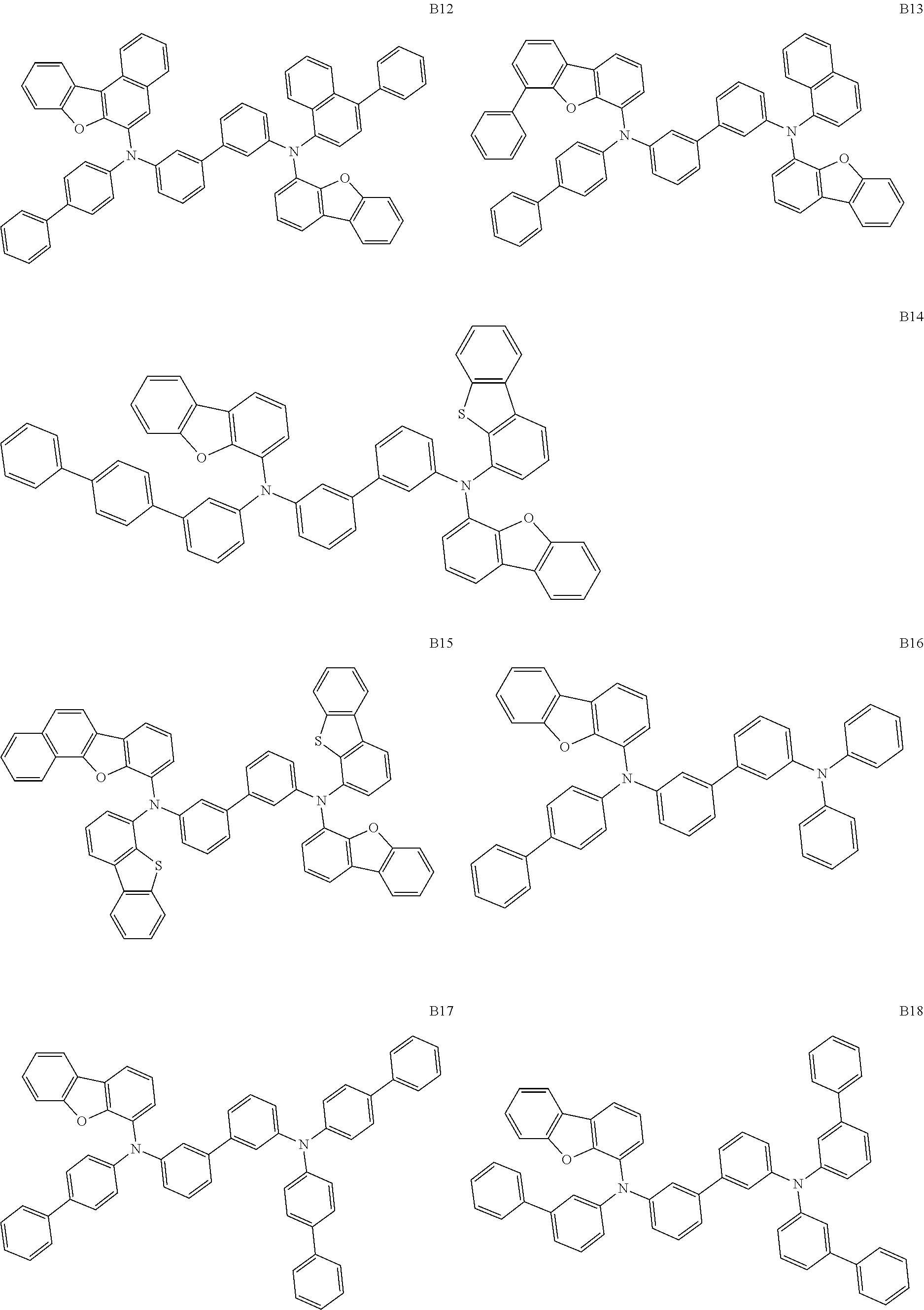

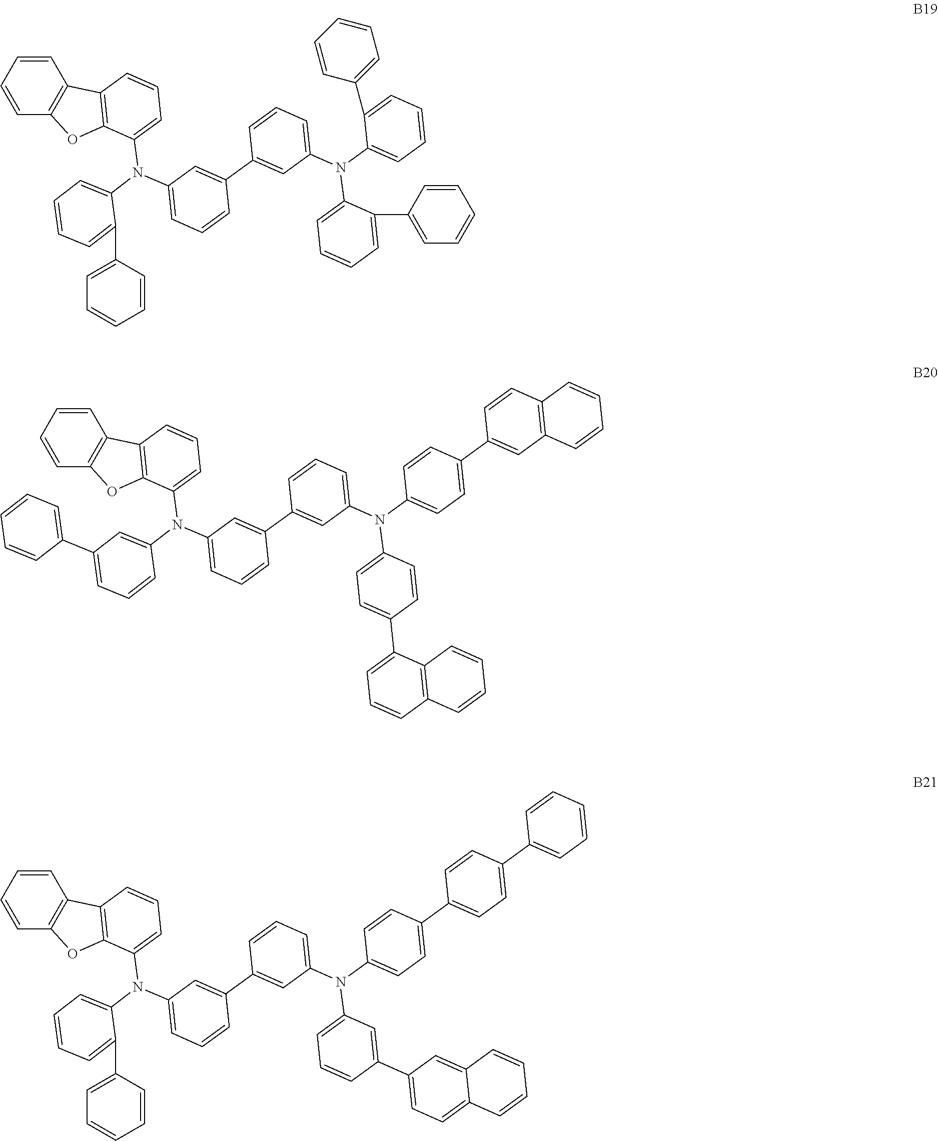 [SMILES: c1ccc(-c2ccccc2N(c2cccc(-c3cccc(N(c4ccccc4-c4ccccc4)c4cccc5c4oc4ccccc45)c3)c2)c2ccccc2-c2ccccc2)cc1 ; c1ccc(-c2cccc(N(c3cccc(-c4cccc(N(c5ccc(-c6ccc7ccccc7c6)cc5)c5ccc(-c6cccc7ccccc67)cc5)c4)c3)c3cccc4c3oc3ccccc34)c2)cc1 ; c1ccc(-c2ccc(-c3ccc(N(c4cccc(-c5cccc(N(c6ccccc6-c6ccccc6)c6cccc7c6oc6ccccc67)c5)c4)c4cccc(-c5ccc6ccccc6c5)c4)cc3)cc2)cc1]

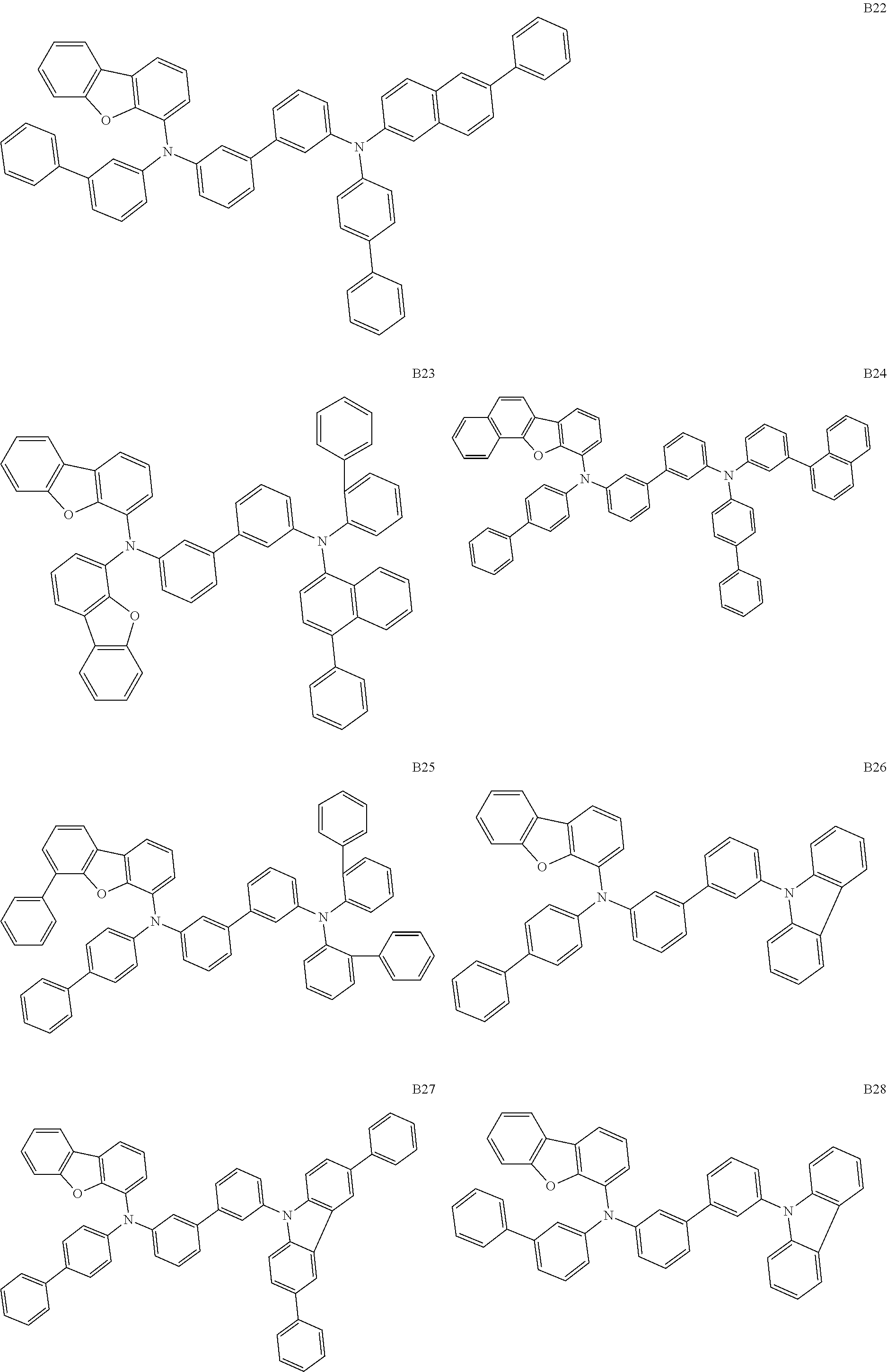

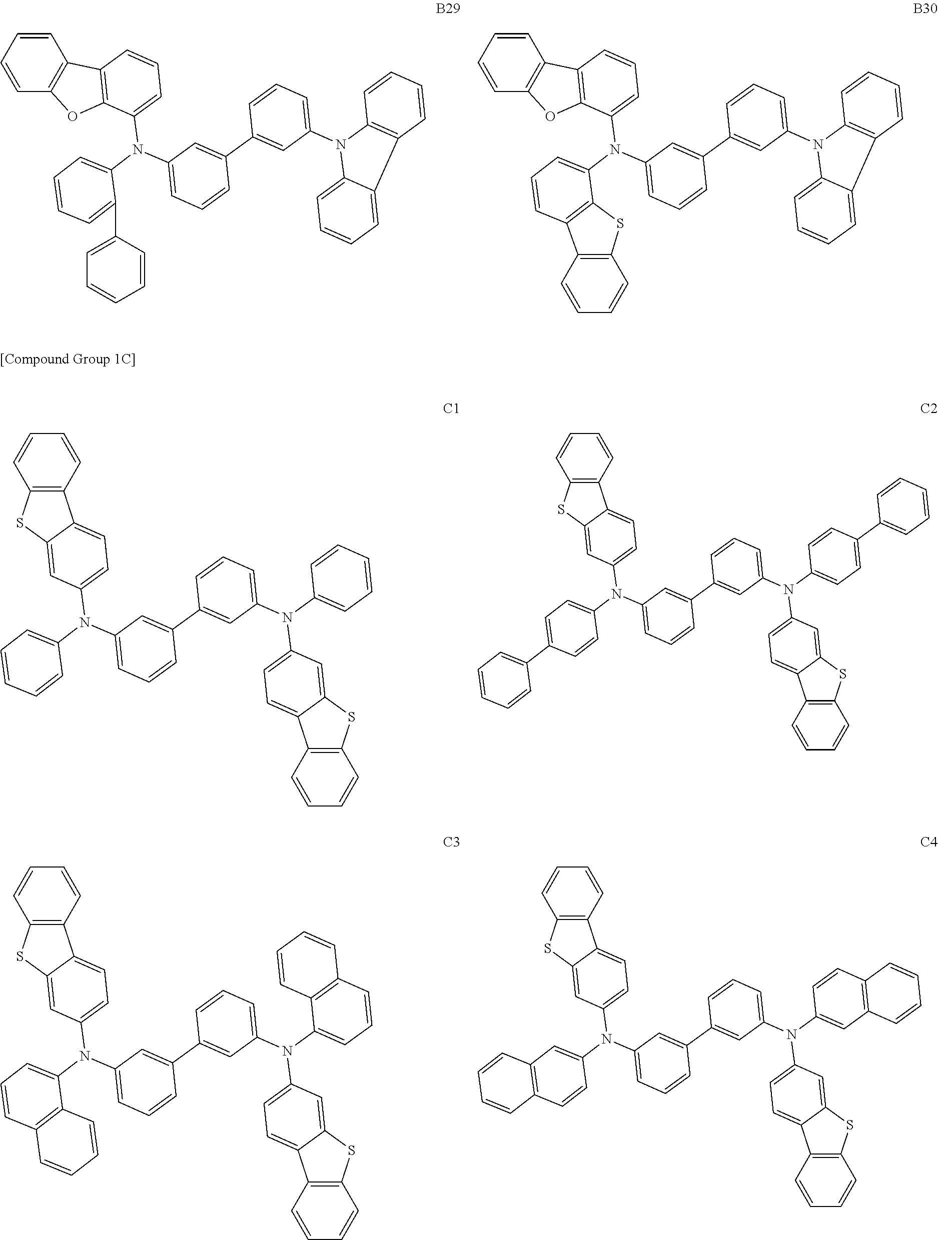

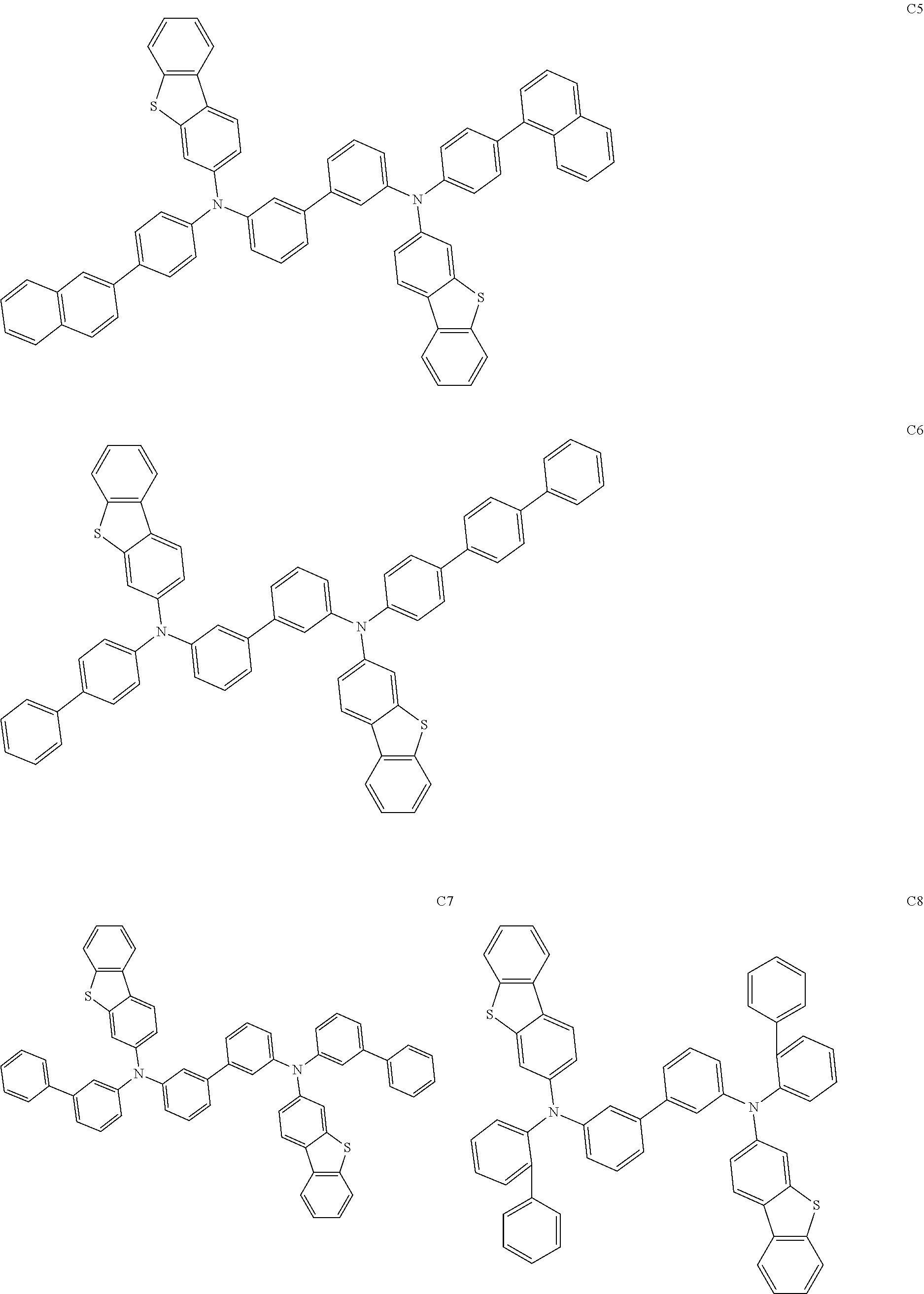 [SMILES: c1cc(-c2cccc(N(c3ccc(-c4cccc5ccccc45)cc3)c3ccc4c(c3)sc3ccccc34)c2)cc(N(c2ccc(-c3ccc4ccccc4c3)cc2)c2ccc3c(c2)sc2ccccc23)c1 ; c1ccc(-c2ccc(-c3ccc(N(c4cccc(-c5cccc(N(c6ccc(-c7ccccc7)cc6)c6ccc7c(c6)sc6ccccc67)c5)c4)c4ccc5c(c4)sc4ccccc45)cc3)cc2)cc1 ; c1ccc(-c2cccc(N(c3cccc(-c4cccc(N(c5cccc(-c6ccccc6)c5)c5ccc6c(c5)sc5ccccc56)c4)c3)c3ccc4c(c3)sc3ccccc34)c2)cc1 ; c1ccc(-c2ccccc2N(c2cccc(-c3cccc(N(c4ccc5c(c4)sc4ccccc45)c4ccccc4-c4ccccc4)c3)c2)c2ccc3c(c2)sc2ccccc23)cc1]

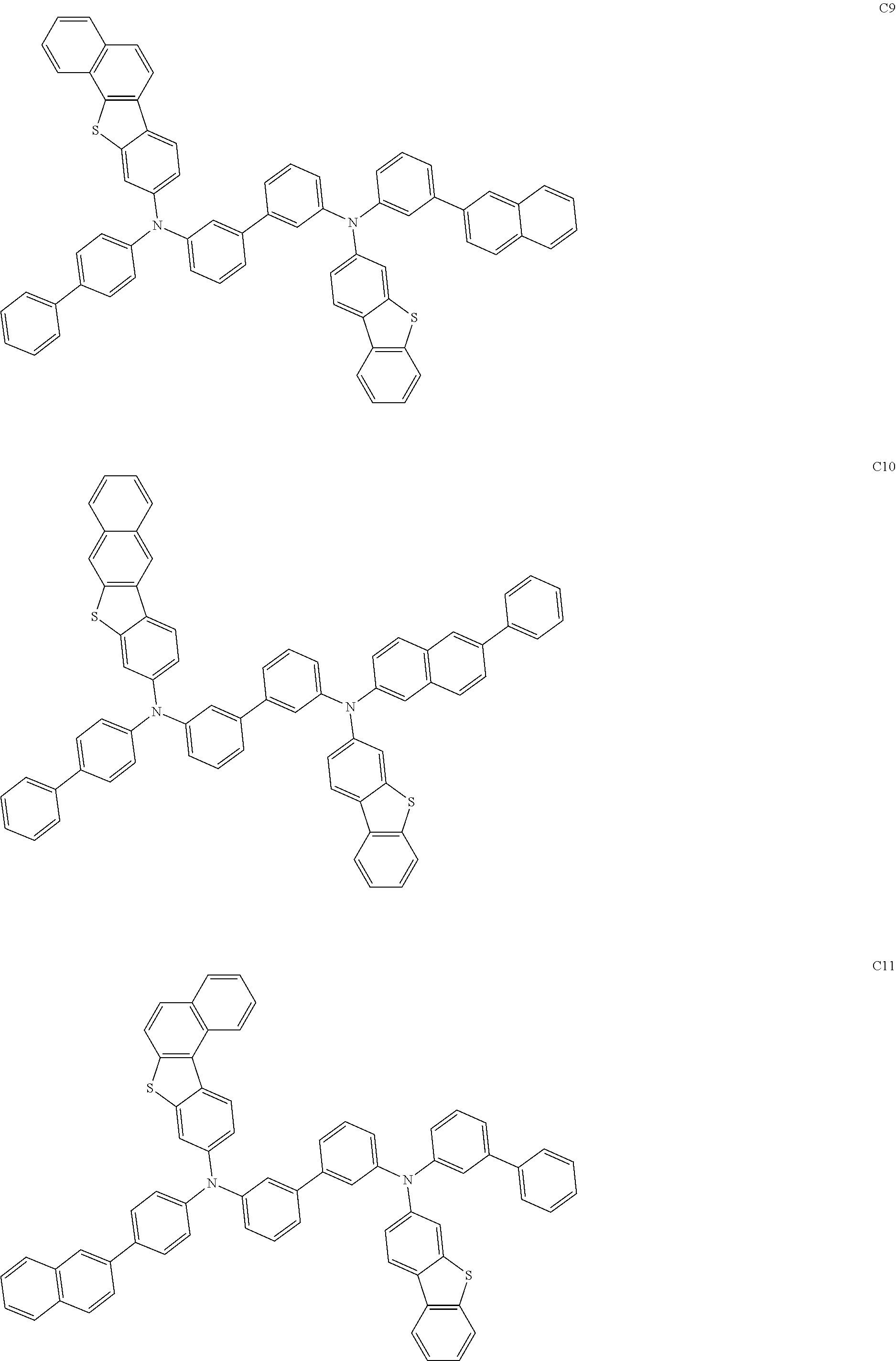 [SMILES: c1ccc(-c2ccc(N(c3cccc(-c4cccc(N(c5cccc(-c6ccc7ccccc7c6)c5)c5ccc6c(c5)sc5ccccc56)c4)c3)c3ccc4c(c3)sc3c5ccccc5ccc43)cc2)cc1 ; c1ccc(-c2ccc(N(c3cccc(-c4cccc(N(c5ccc6cc(-c7ccccc7)ccc6c5)c5ccc6c(c5)sc5ccccc56)c4)c3)c3ccc4c(c3)sc3cc5ccccc5cc34)cc2)cc1 ; c1ccc(-c2cccc(N(c3cccc(-c4cccc(N(c5ccc(-c6ccc7ccccc7c6)cc5)c5ccc6c(c5)sc5ccc7ccccc7c56)c4)c3)c3ccc4c(c3)sc3ccccc34)c2)cc1]

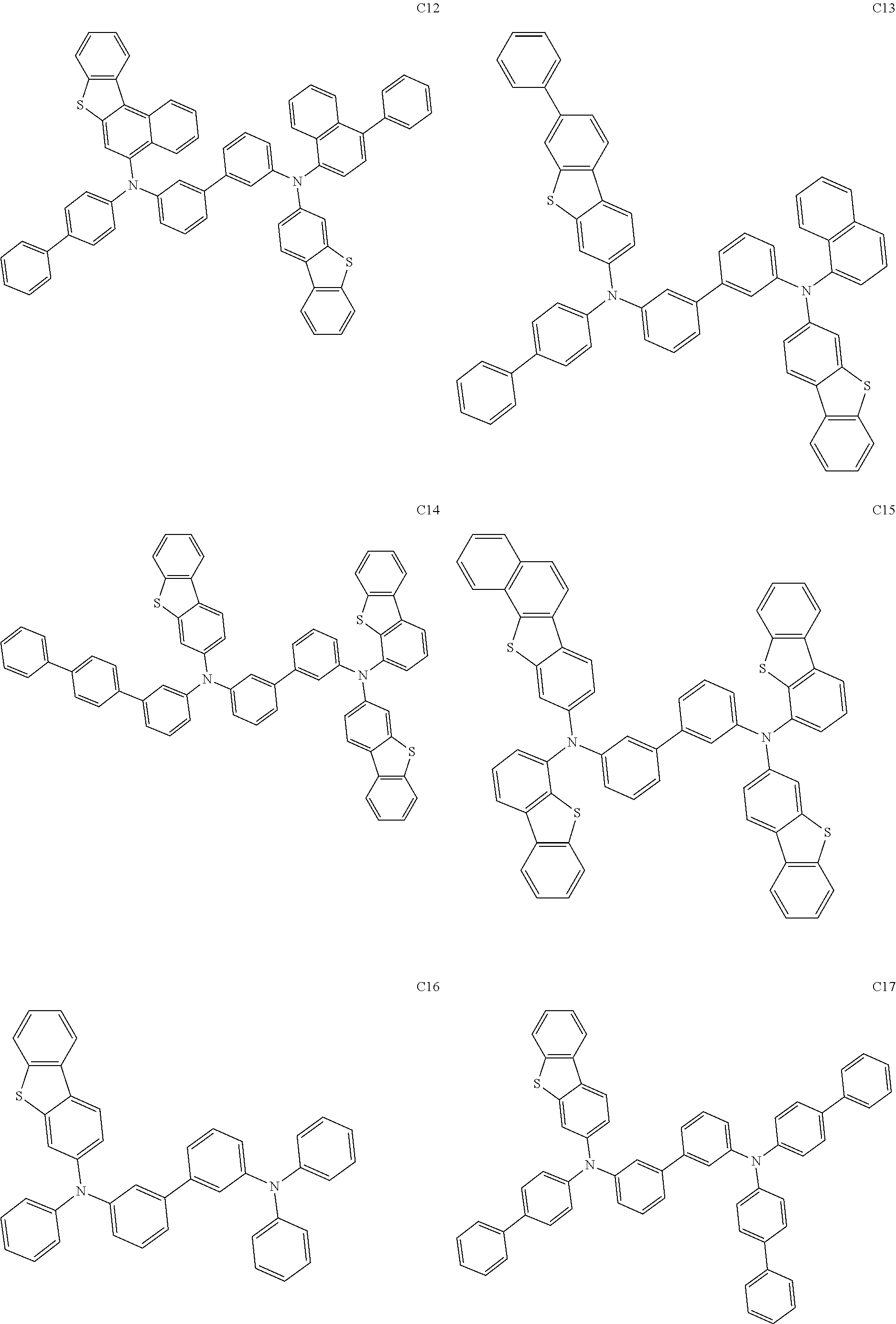

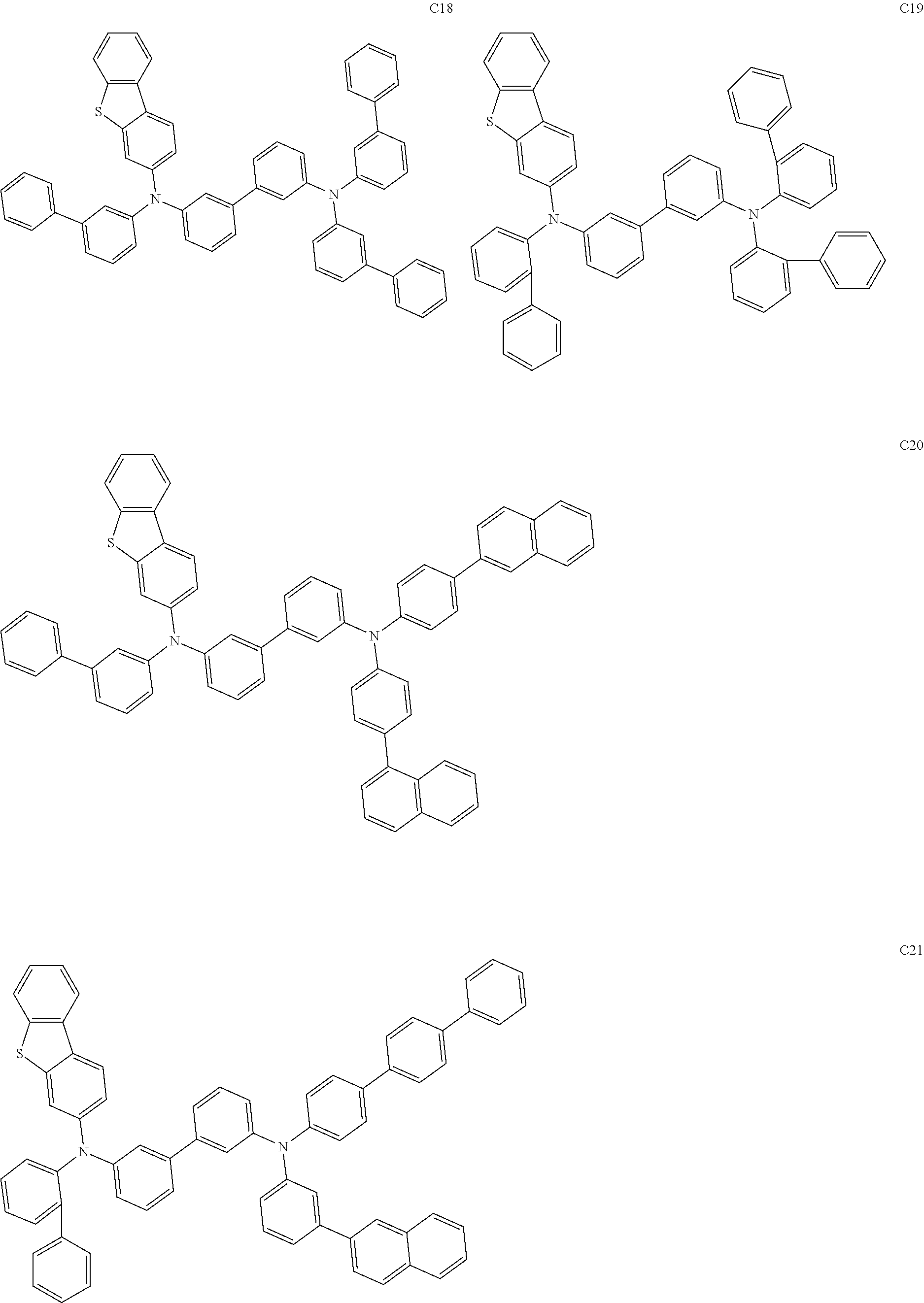

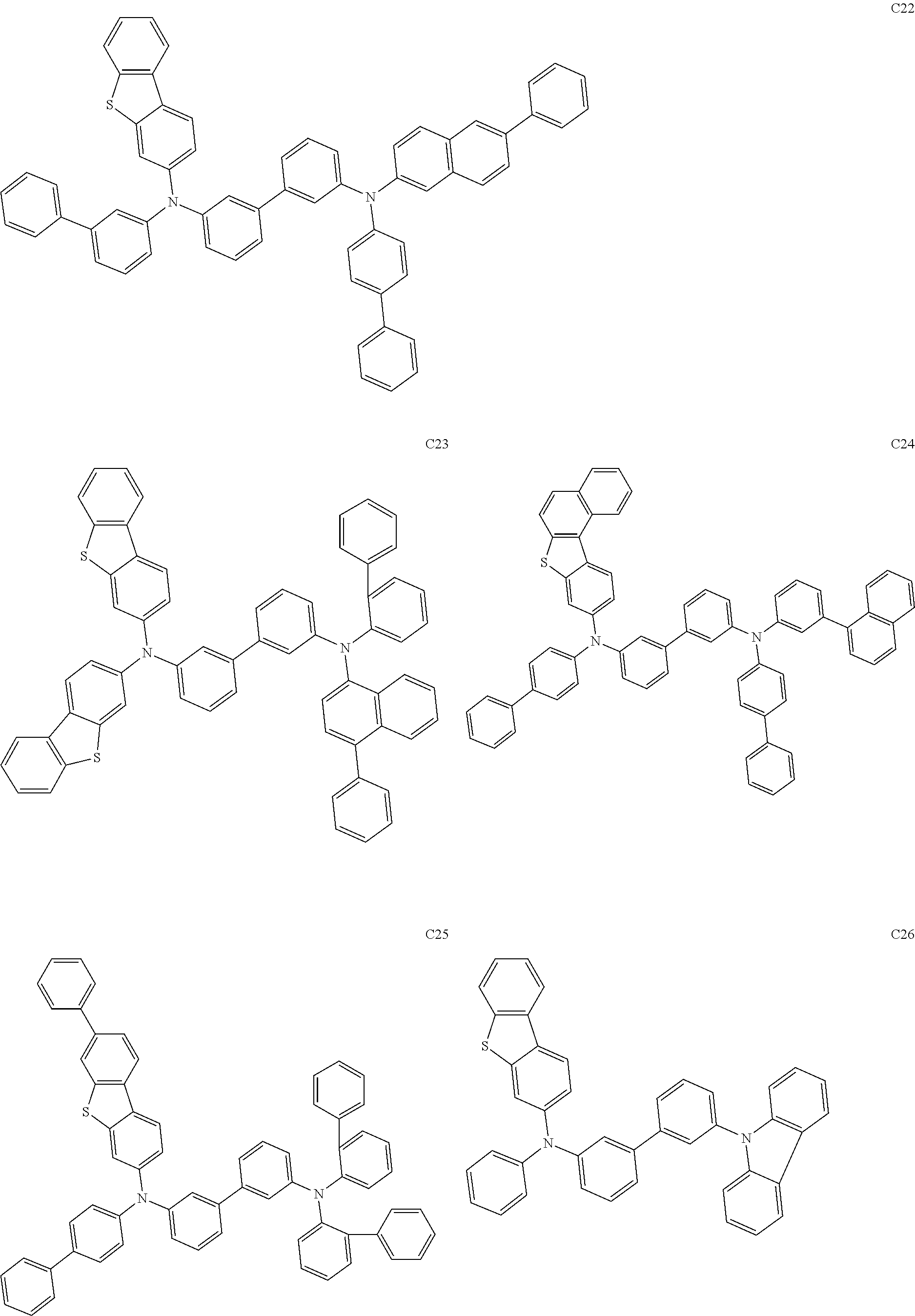

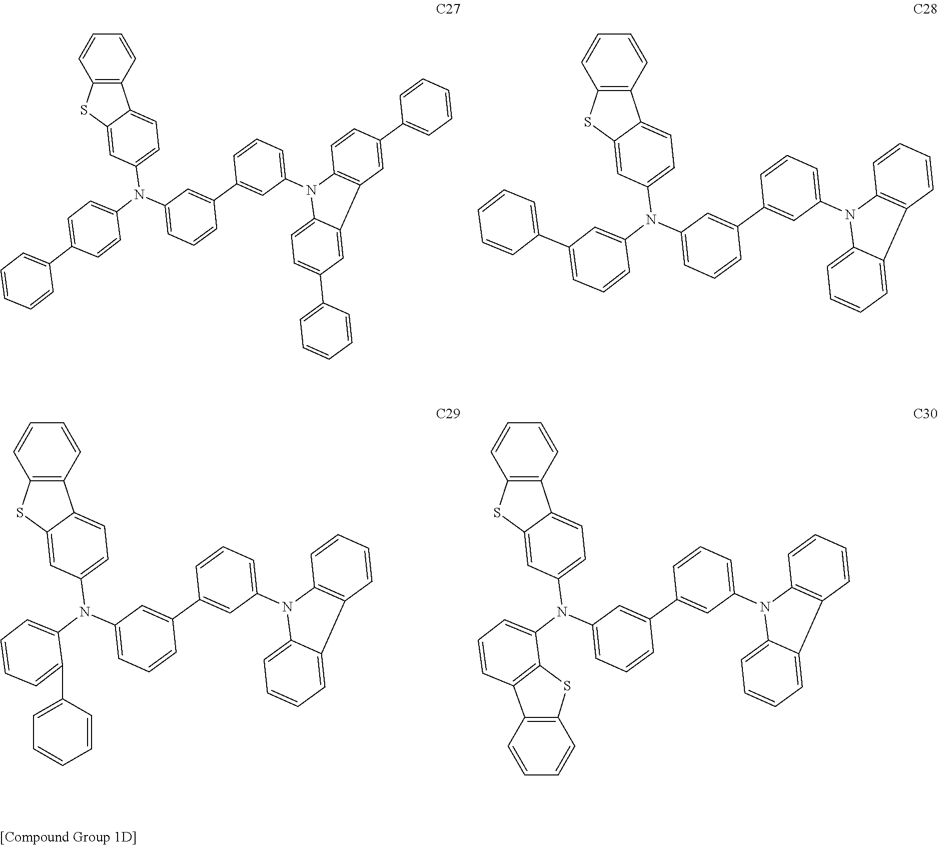 [SMILES: c1ccc(-c2ccc(N(c3cccc(-c4cccc(-n5c6ccc(-c7ccccc7)cc6c6cc(-c7ccccc7)ccc65)c4)c3)c3ccc4c(c3)sc3ccccc34)cc2)cc1 ; c1ccc(-c2cccc(N(c3cccc(-c4cccc(-n5c6ccccc6c6ccccc65)c4)c3)c3ccc4c(c3)sc3ccccc34)c2)cc1 ; c1ccc(-c2ccccc2N(c2cccc(-c3cccc(-n4c5ccccc5c5ccccc54)c3)c2)c2ccc3c(c2)sc2ccccc23)cc1 ; c1cc(-c2cccc(-n3c4ccccc4c4ccccc43)c2)cc(N(c2ccc3c(c2)sc2ccccc23)c2cccc3c2sc2ccccc23)c1]

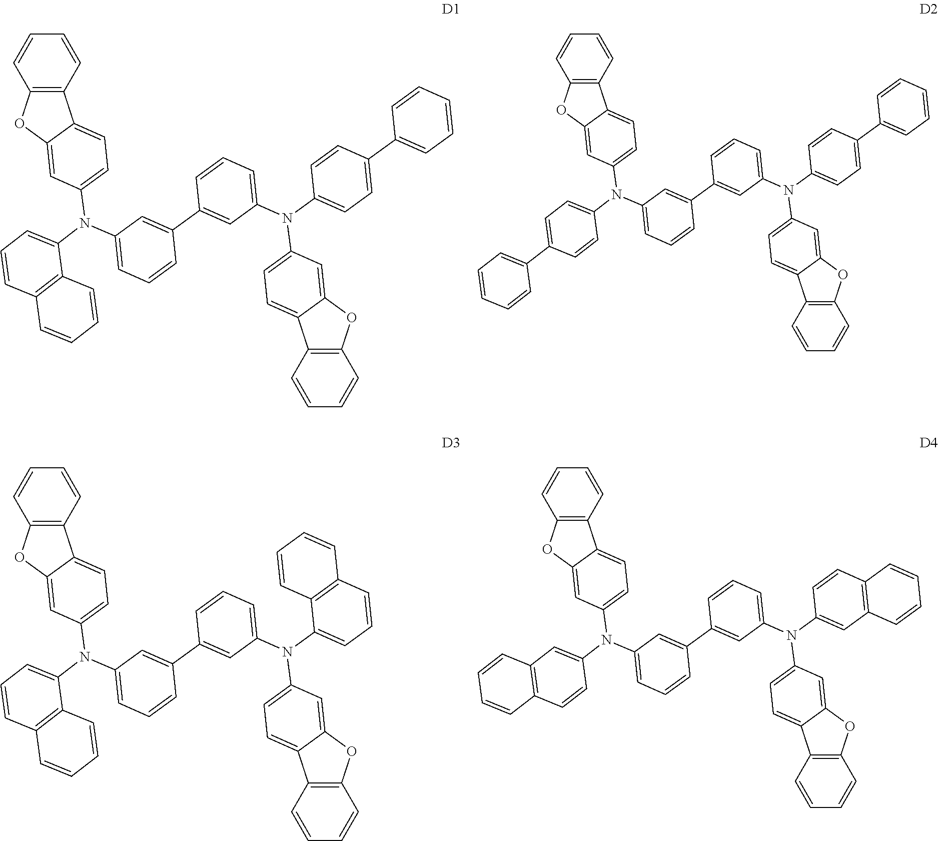 [SMILES: c1ccc(-c2ccc(N(c3cccc(-c4cccc(N(c5ccc6c(c5)oc5ccccc56)c5cccc6ccccc56)c4)c3)c3ccc4c(c3)oc3ccccc34)cc2)cc1 ; c1ccc(-c2ccc(N(c3cccc(-c4cccc(N(c5ccc(-c6ccccc6)cc5)c5ccc6c(c5)oc5ccccc56)c4)c3)c3ccc4c(c3)oc3ccccc34)cc2)cc1 ; c1cc(-c2cccc(N(c3ccc4c(c3)oc3ccccc34)c3cccc4ccccc34)c2)cc(N(c2ccc3c(c2)oc2ccccc23)c2cccc3ccccc23)c1 ; c1cc(-c2cccc(N(c3ccc4ccccc4c3)c3ccc4c(c3)oc3ccccc34)c2)cc(N(c2ccc3ccccc3c2)c2ccc3c(c2)oc2ccccc23)c1]

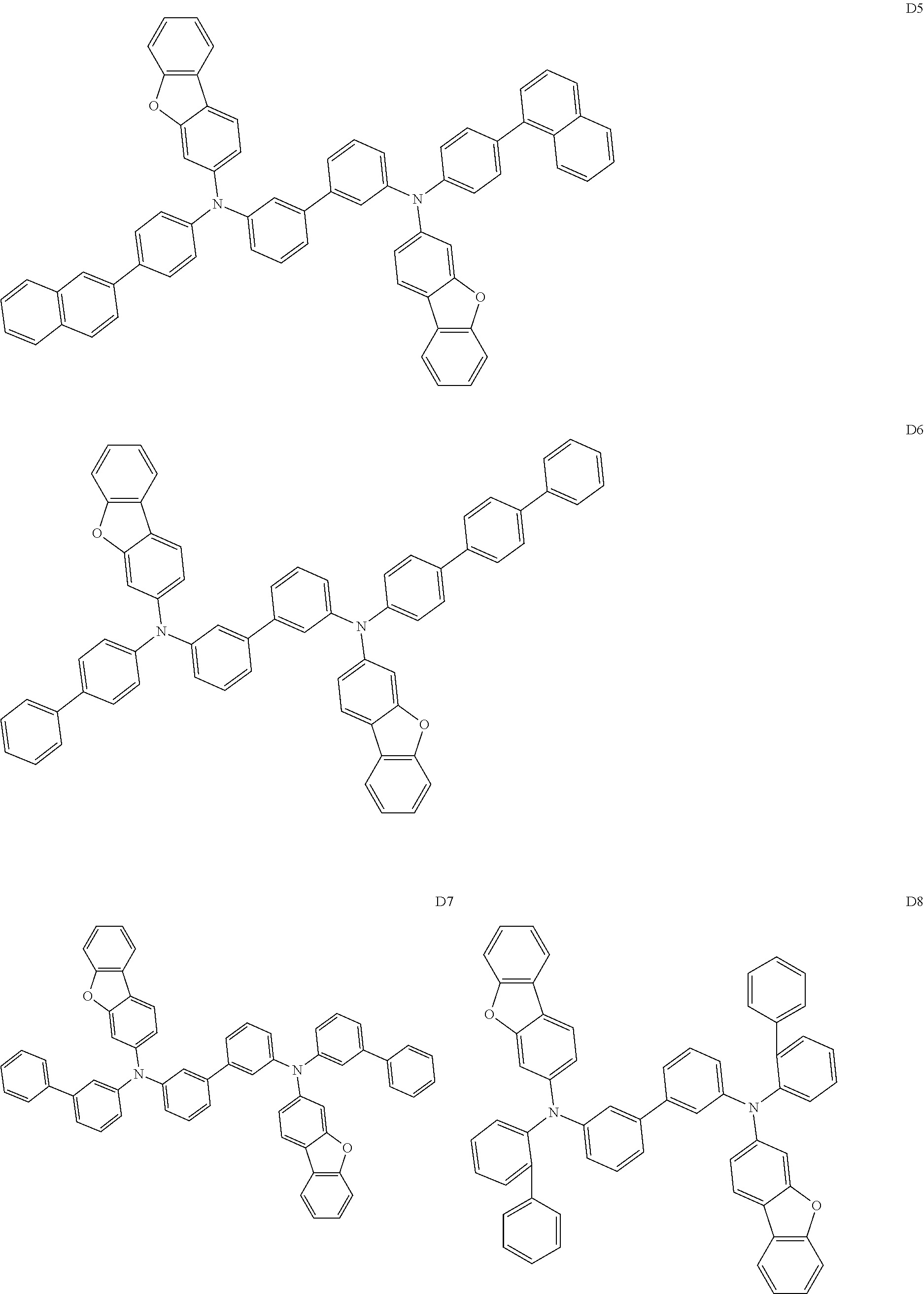 [SMILES: c1cc(-c2cccc(N(c3ccc(-c4cccc5ccccc45)cc3)c3ccc4c(c3)oc3ccccc34)c2)cc(N(c2ccc(-c3ccc4ccccc4c3)cc2)c2ccc3c(c2)oc2ccccc23)c1 ; c1ccc(-c2ccc(-c3ccc(N(c4cccc(-c5cccc(N(c6ccc(-c7ccccc7)cc6)c6ccc7c(c6)oc6ccccc67)c5)c4)c4ccc5c(c4)oc4ccccc45)cc3)cc2)cc1 ; c1ccc(-c2cccc(N(c3cccc(-c4cccc(N(c5cccc(-c6ccccc6)c5)c5ccc6c(c5)oc5ccccc56)c4)c3)c3ccc4c(c3)oc3ccccc34)c2)cc1 ; c1ccc(-c2ccccc2N(c2cccc(-c3cccc(N(c4ccc5c(c4)oc4ccccc45)c4ccccc4-c4ccccc4)c3)c2)c2ccc3c(c2)oc2ccccc23)cc1]

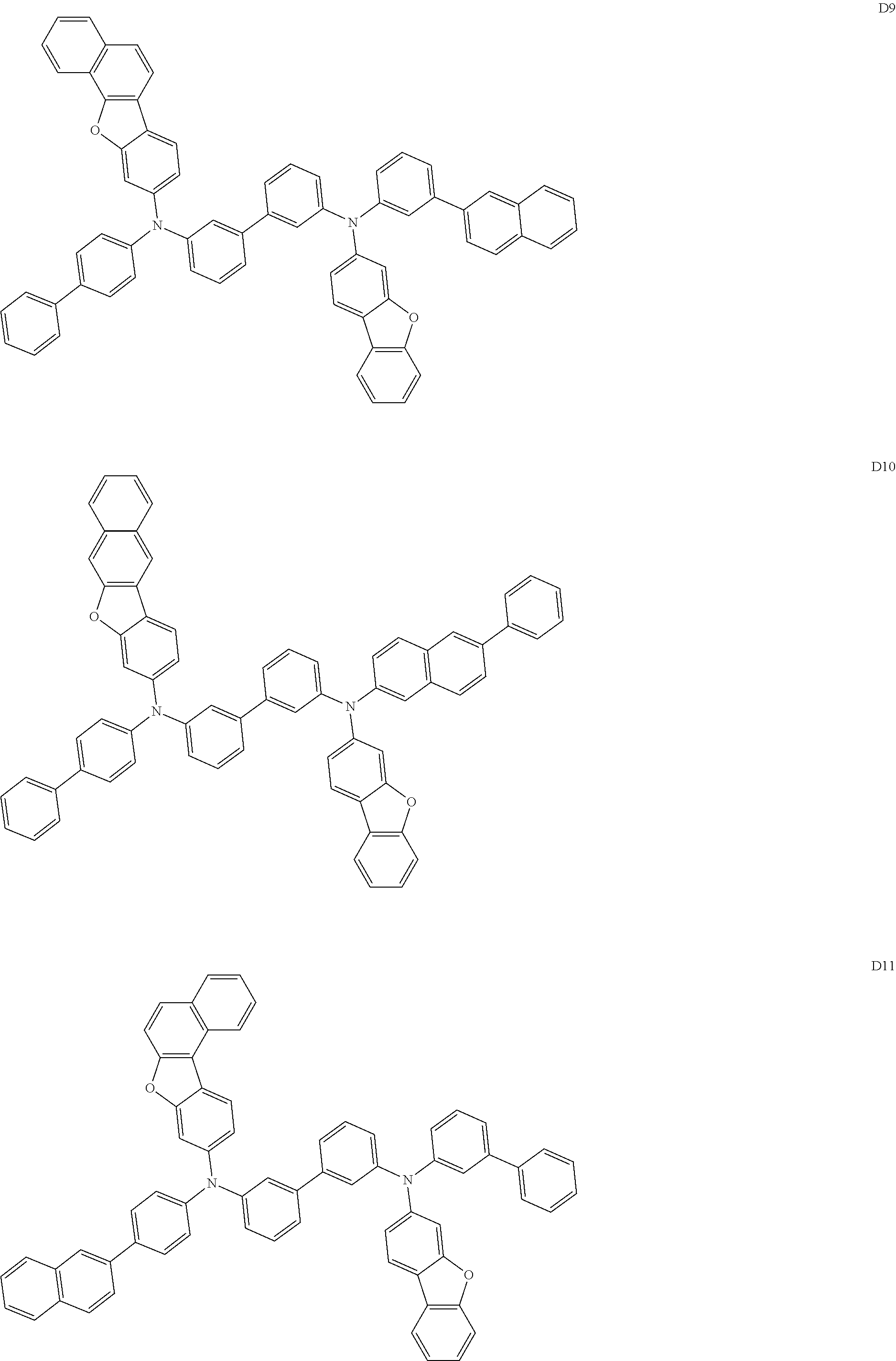 [SMILES: c1ccc(-c2ccc(N(c3cccc(-c4cccc(N(c5cccc(-c6ccc7ccccc7c6)c5)c5ccc6c(c5)oc5ccccc56)c4)c3)c3ccc4c(c3)oc3c5ccccc5ccc43)cc2)cc1 ; c1ccc(-c2ccc(N(c3cccc(-c4cccc(N(c5ccc6cc(-c7ccccc7)ccc6c5)c5ccc6c(c5)oc5ccccc56)c4)c3)c3ccc4c(c3)oc3cc5ccccc5cc34)cc2)cc1 ; c1ccc(-c2cccc(N(c3cccc(-c4cccc(N(c5ccc(-c6ccc7ccccc7c6)cc5)c5ccc6c(c5)oc5ccc7ccccc7c56)c4)c3)c3ccc4c(c3)oc3ccccc34)c2)cc1]

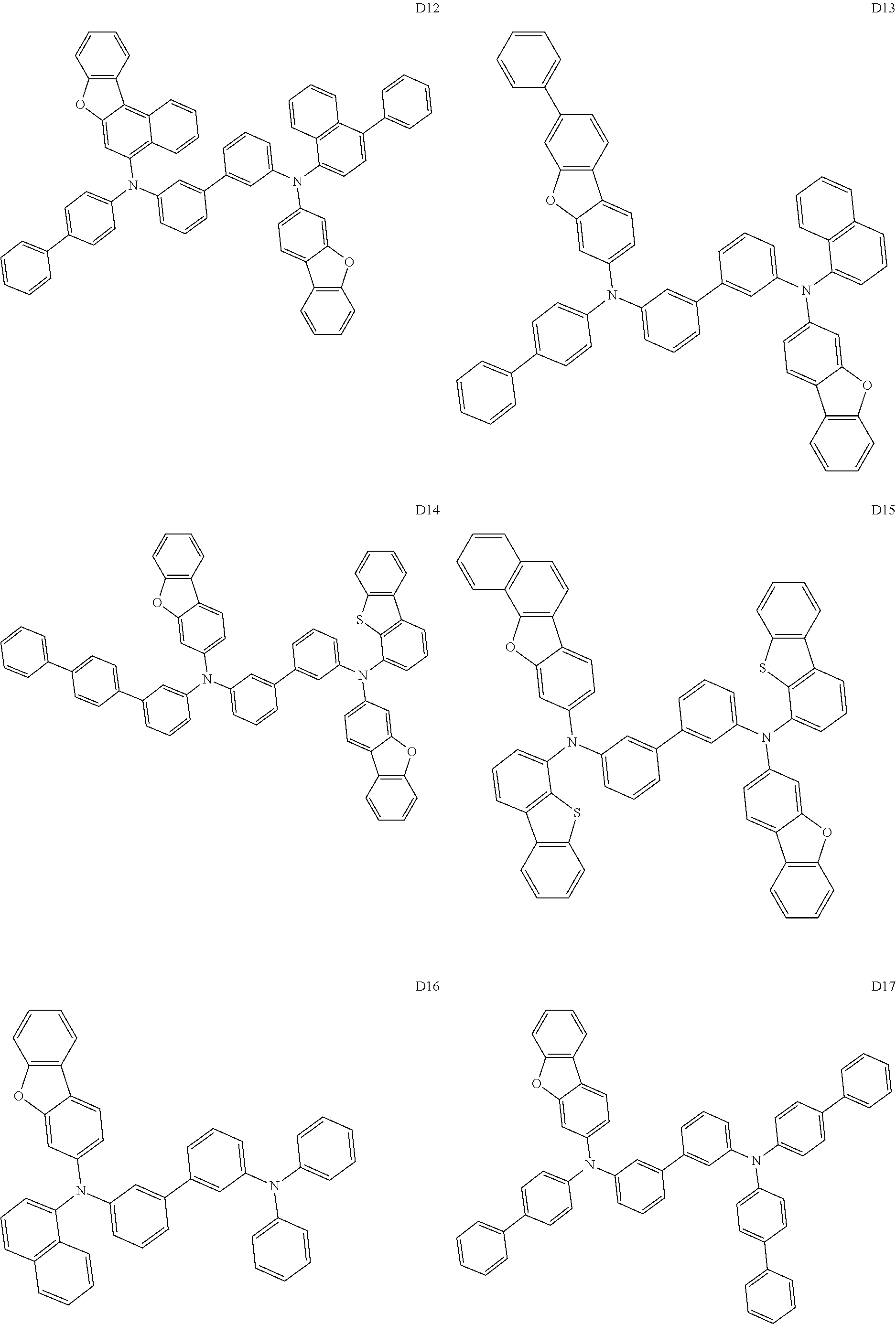

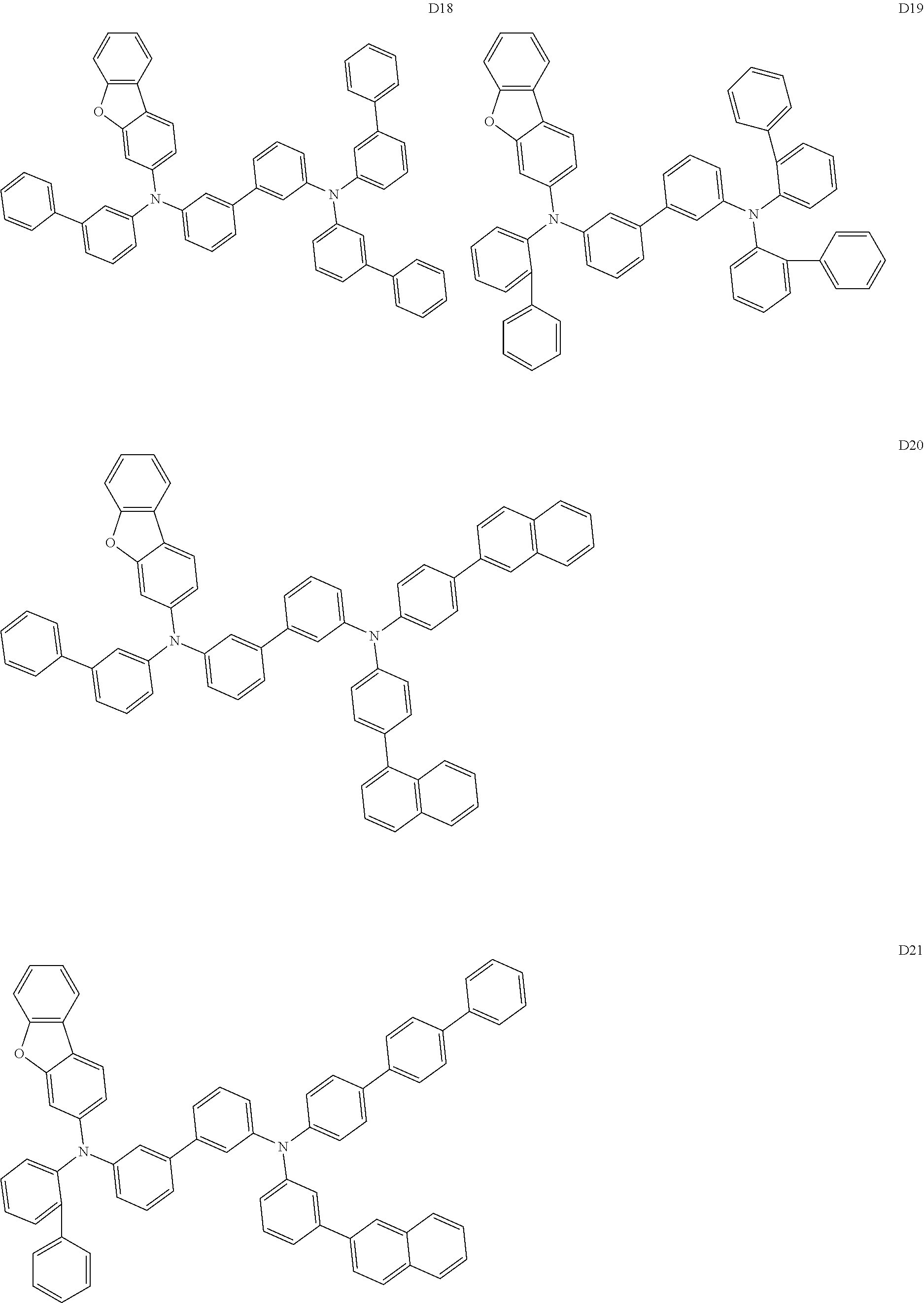

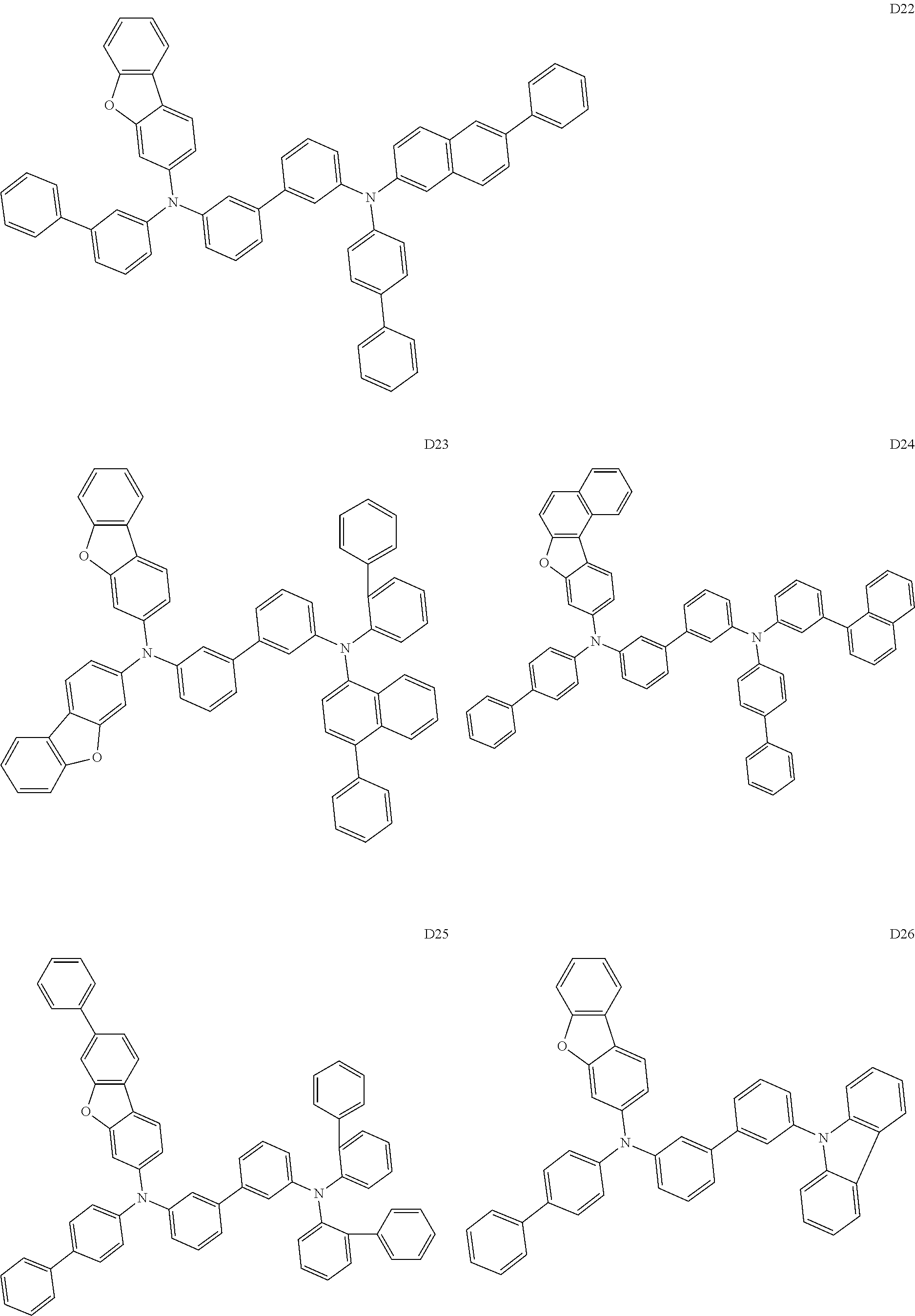

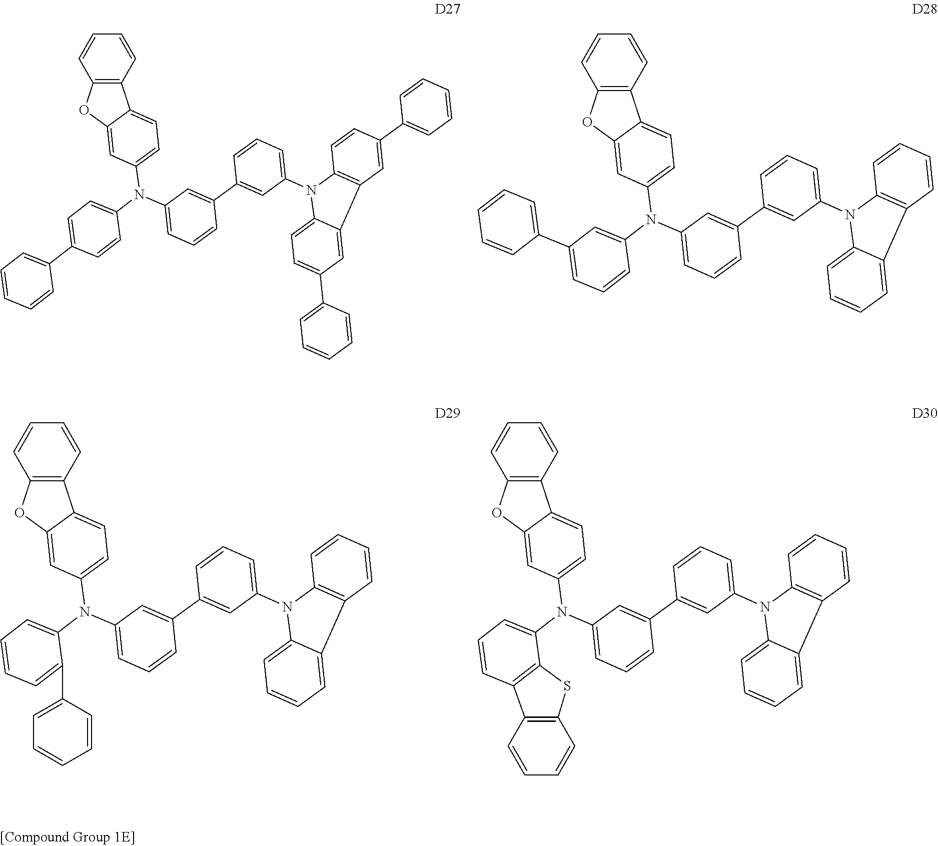 [SMILES: c1ccc(-c2ccc(N(c3cccc(-c4cccc(-n5c6ccc(-c7ccccc7)cc6c6cc(-c7ccccc7)ccc65)c4)c3)c3ccc4c(c3)oc3ccccc34)cc2)cc1 ; c1ccc(-c2cccc(N(c3cccc(-c4cccc(-n5c6ccccc6c6ccccc65)c4)c3)c3ccc4c(c3)oc3ccccc34)c2)cc1 ; c1ccc(-c2ccccc2N(c2cccc(-c3cccc(-n4c5ccccc5c5ccccc54)c3)c2)c2ccc3c(c2)oc2ccccc23)cc1 ; c1cc(-c2cccc(-n3c4ccccc4c4ccccc43)c2)cc(N(c2ccc3c(c2)oc2ccccc23)c2cccc3c2sc2ccccc23)c1]

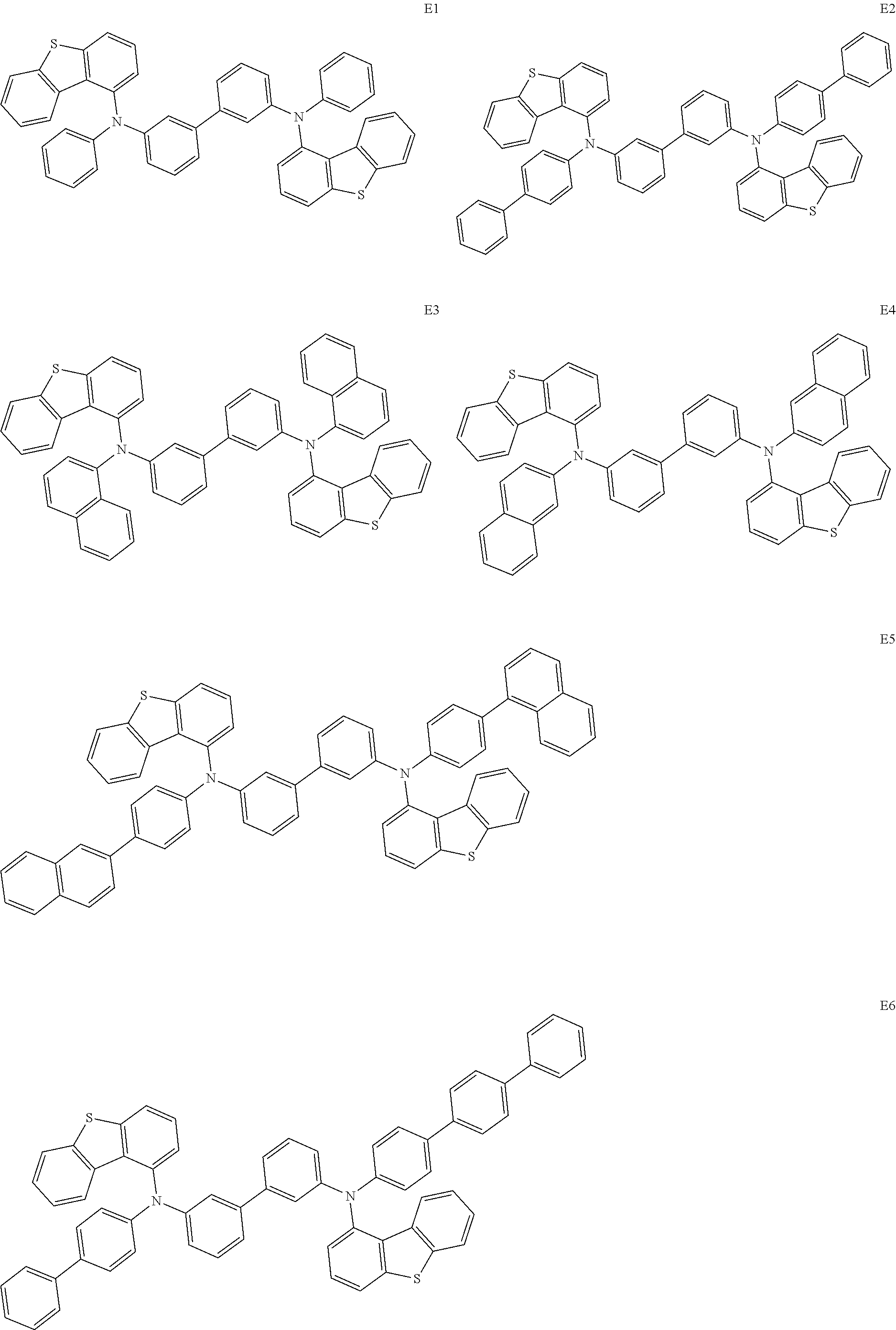

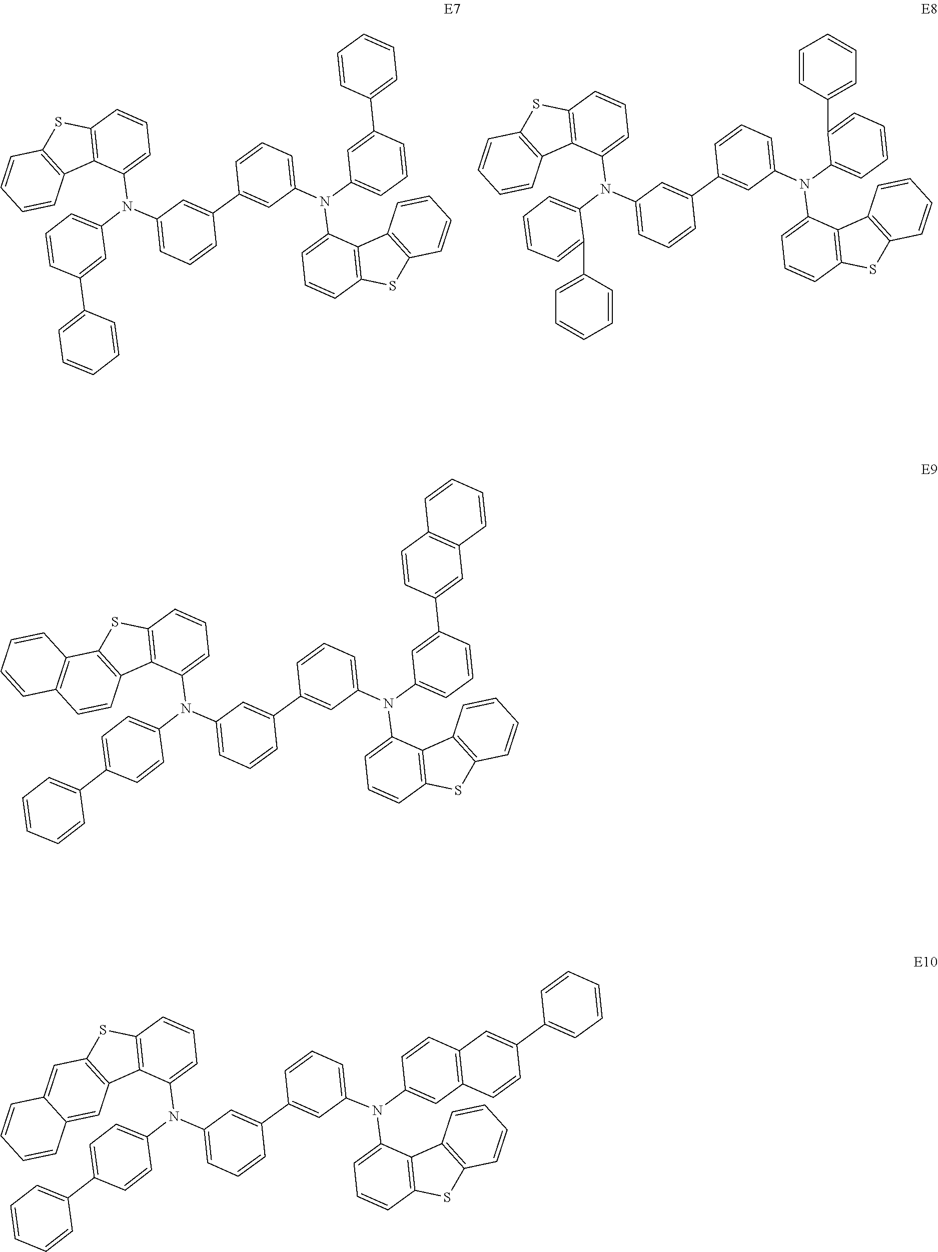 [SMILES: c1ccc(-c2cccc(N(c3cccc(-c4cccc(N(c5cccc(-c6ccccc6)c5)c5cccc6sc7ccccc7c56)c4)c3)c3cccc4sc5ccccc5c34)c2)cc1 ; c1ccc(-c2ccccc2N(c2cccc(-c3cccc(N(c4ccccc4-c4ccccc4)c4cccc5sc6ccccc6c45)c3)c2)c2cccc3sc4ccccc4c23)cc1 ; c1ccc(-c2ccc(N(c3cccc(-c4cccc(N(c5cccc(-c6ccc7ccccc7c6)c5)c5cccc6sc7ccccc7c56)c4)c3)c3cccc4sc5c6ccccc6ccc5c34)cc2)cc1 ; c1ccc(-c2ccc(N(c3cccc(-c4cccc(N(c5ccc6cc(-c7ccccc7)ccc6c5)c5cccc6sc7ccccc7c56)c4)c3)c3cccc4sc5cc6ccccc6cc5c34)cc2)cc1]

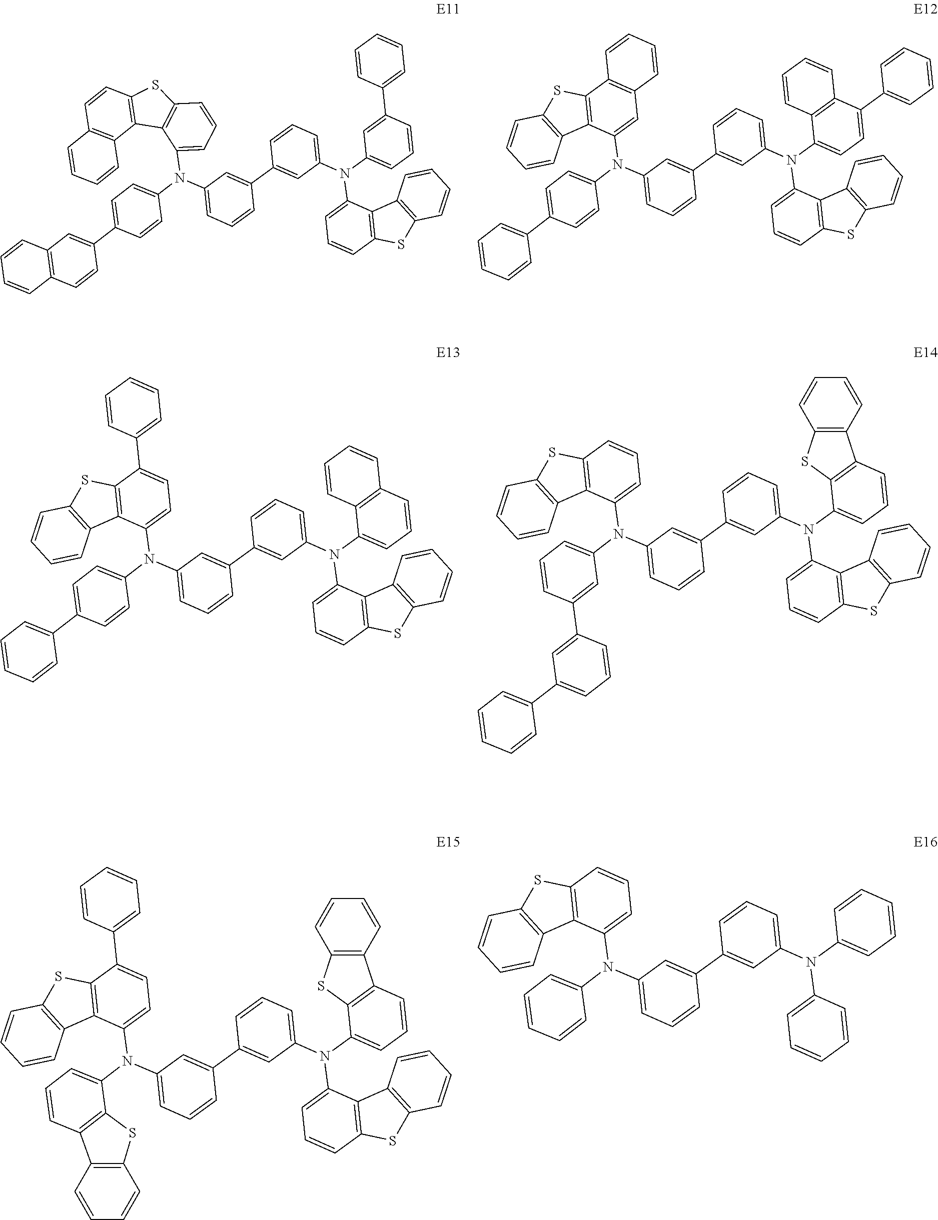

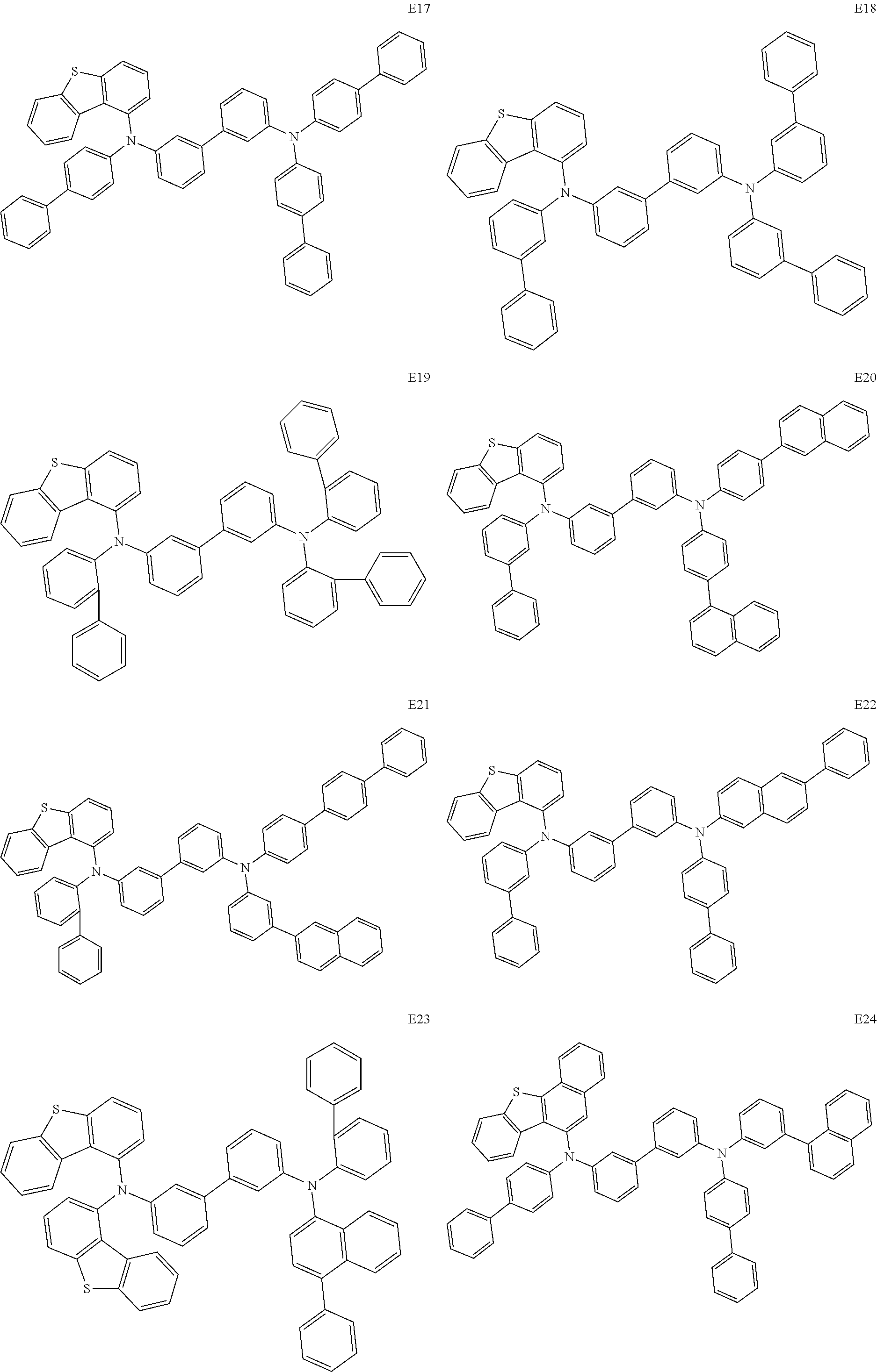

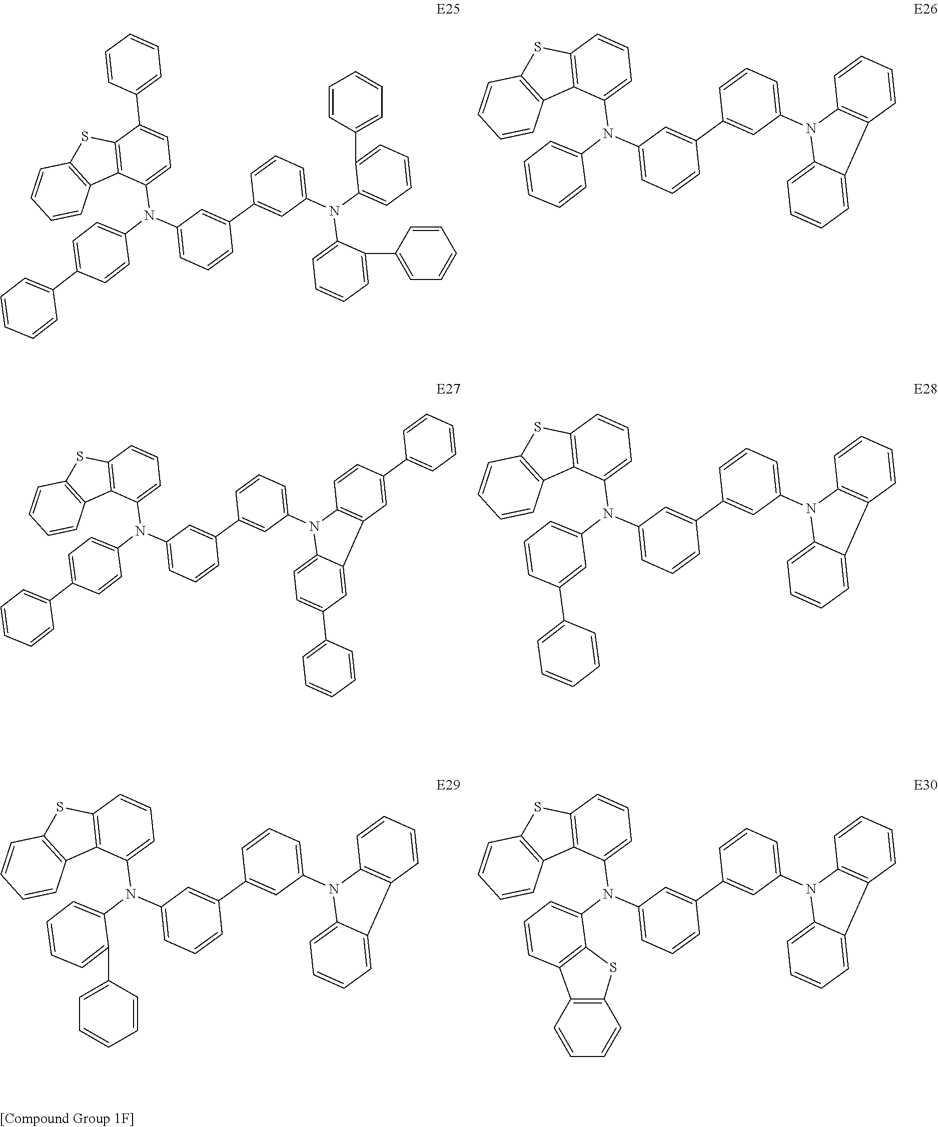

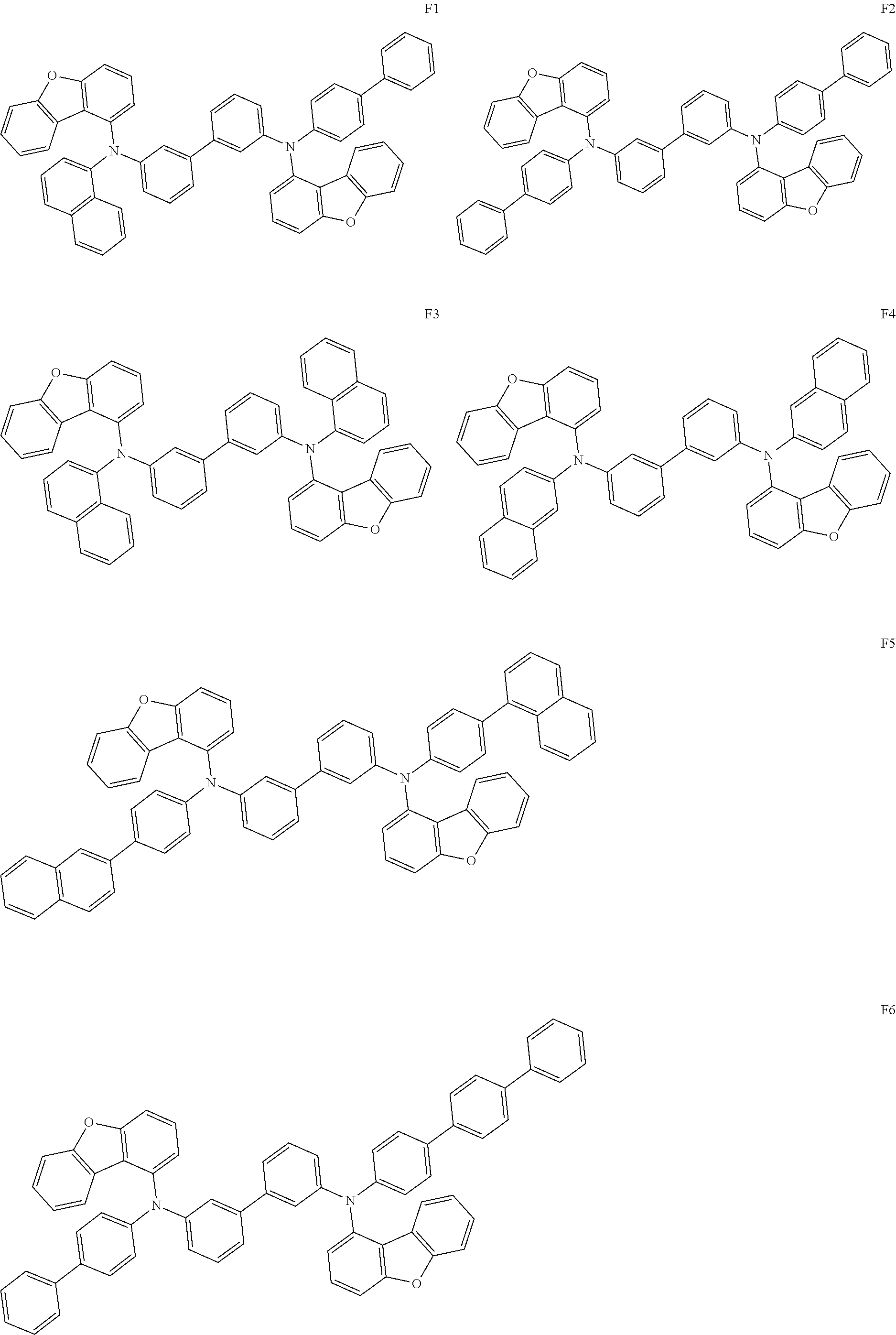

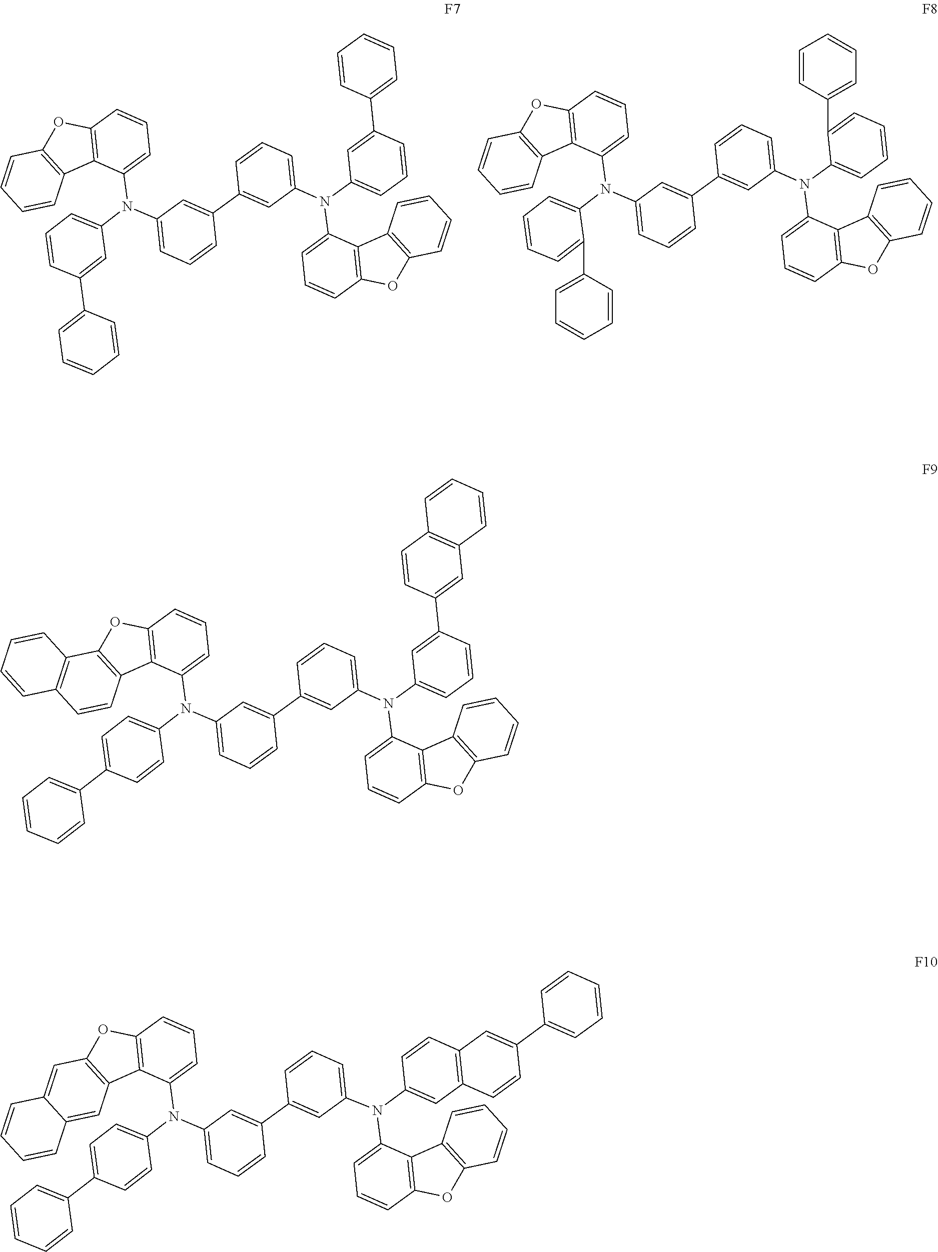 [SMILES: c1ccc(-c2cccc(N(c3cccc(-c4cccc(N(c5cccc(-c6ccccc6)c5)c5cccc6oc7ccccc7c56)c4)c3)c3cccc4oc5ccccc5c34)c2)cc1 ; c1ccc(-c2ccccc2N(c2cccc(-c3cccc(N(c4ccccc4-c4ccccc4)c4cccc5oc6ccccc6c45)c3)c2)c2cccc3oc4ccccc4c23)cc1 ; c1ccc(-c2ccc(N(c3cccc(-c4cccc(N(c5cccc(-c6ccc7ccccc7c6)c5)c5cccc6oc7ccccc7c56)c4)c3)c3cccc4oc5c6ccccc6ccc5c34)cc2)cc1 ; c1ccc(-c2ccc(N(c3cccc(-c4cccc(N(c5ccc6cc(-c7ccccc7)ccc6c5)c5cccc6oc7ccccc7c56)c4)c3)c3cccc4oc5cc6ccccc6cc5c34)cc2)cc1]

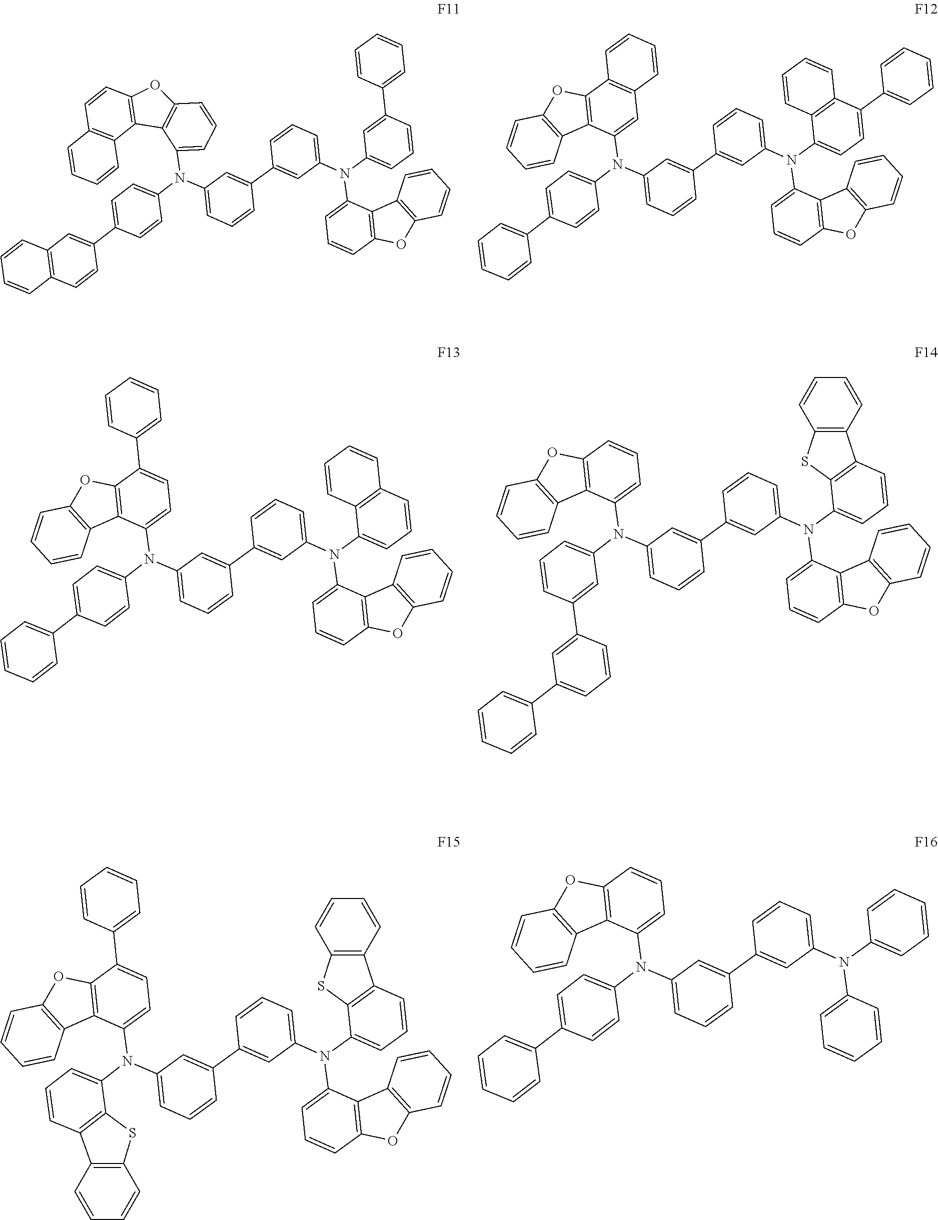

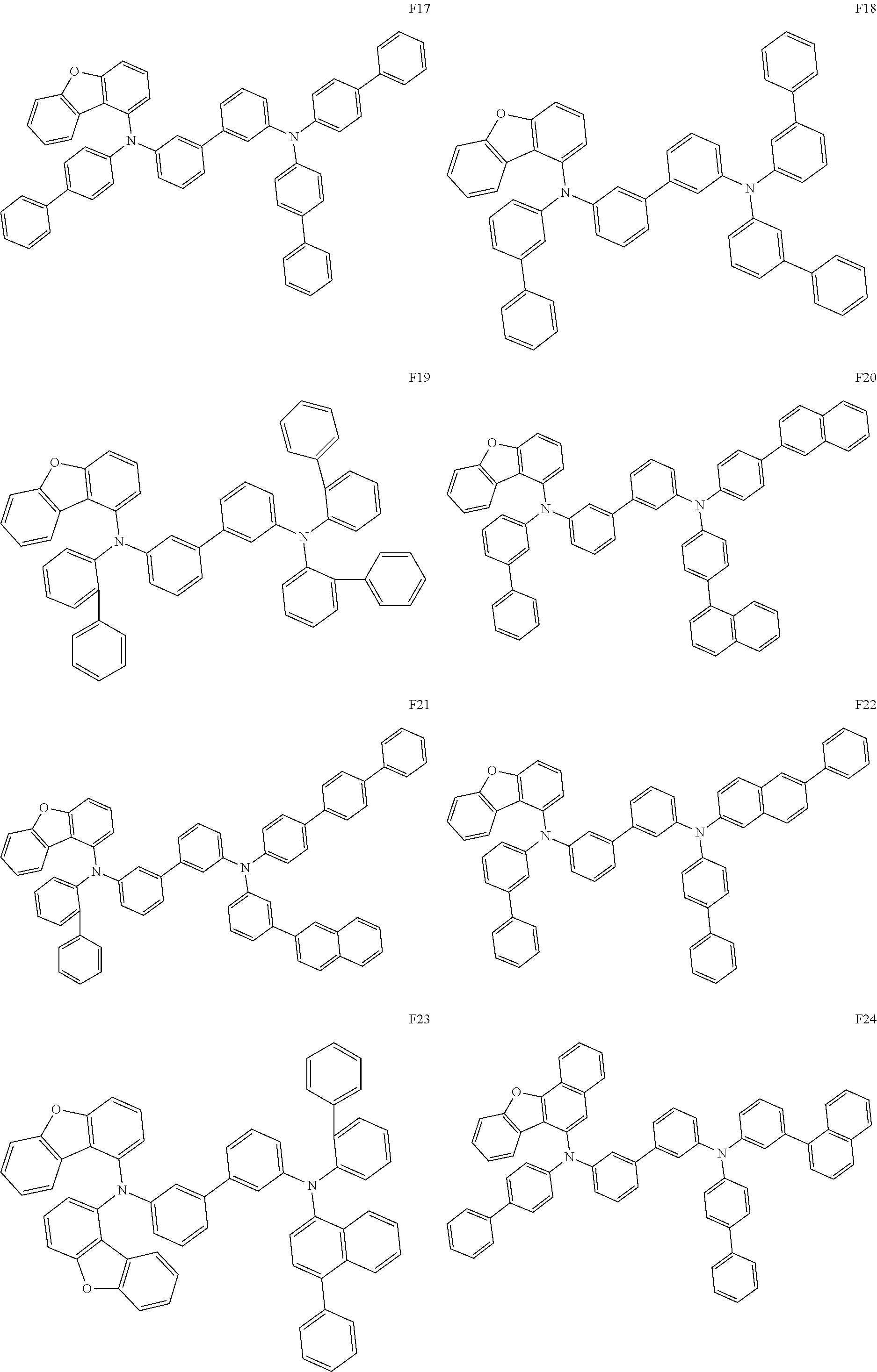

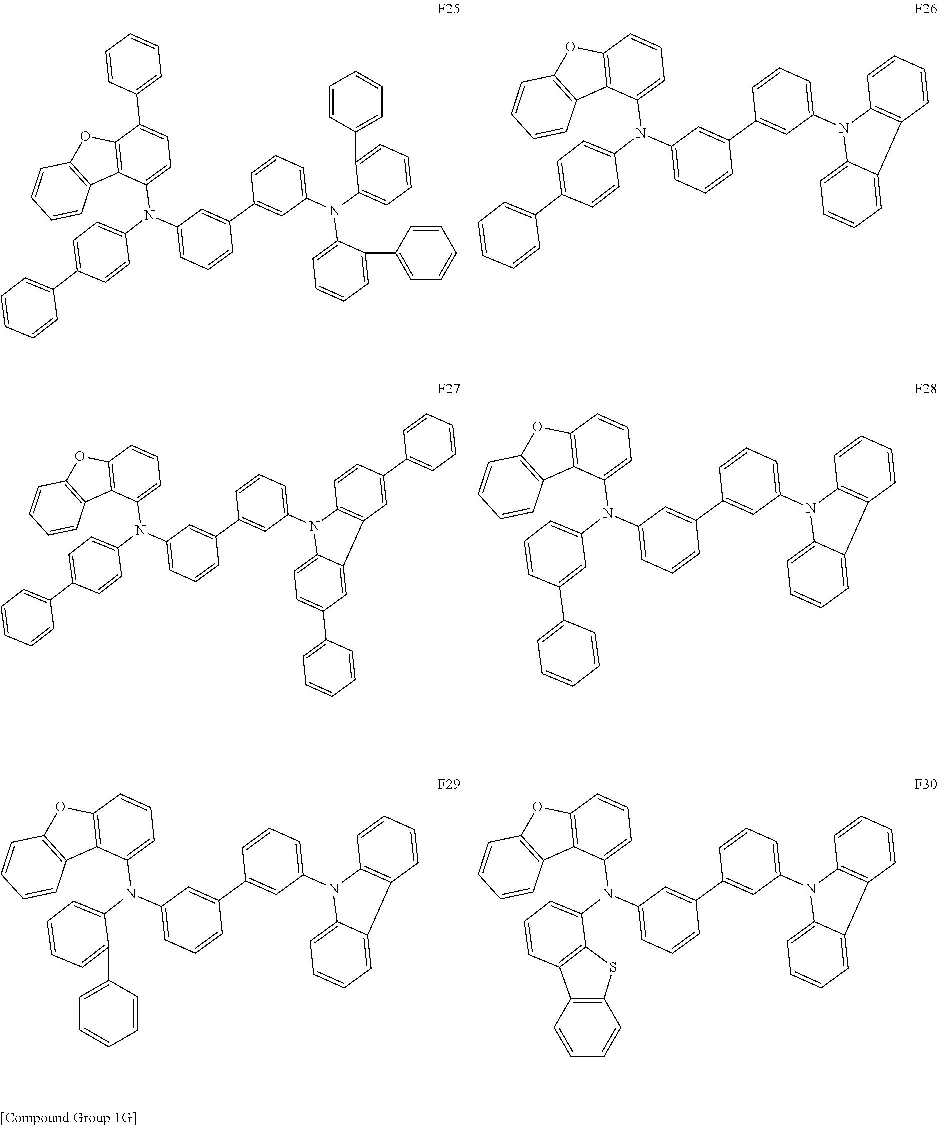

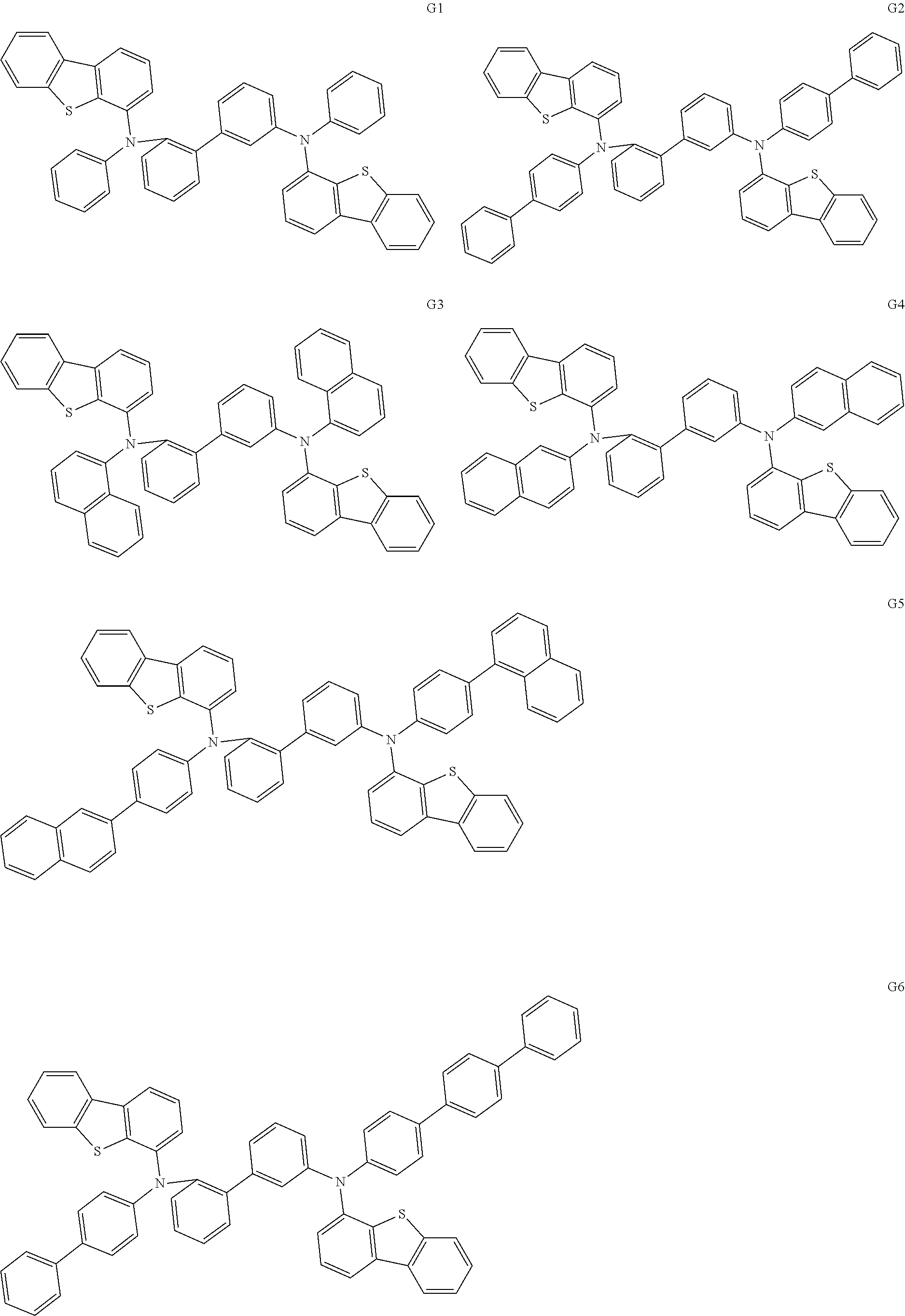

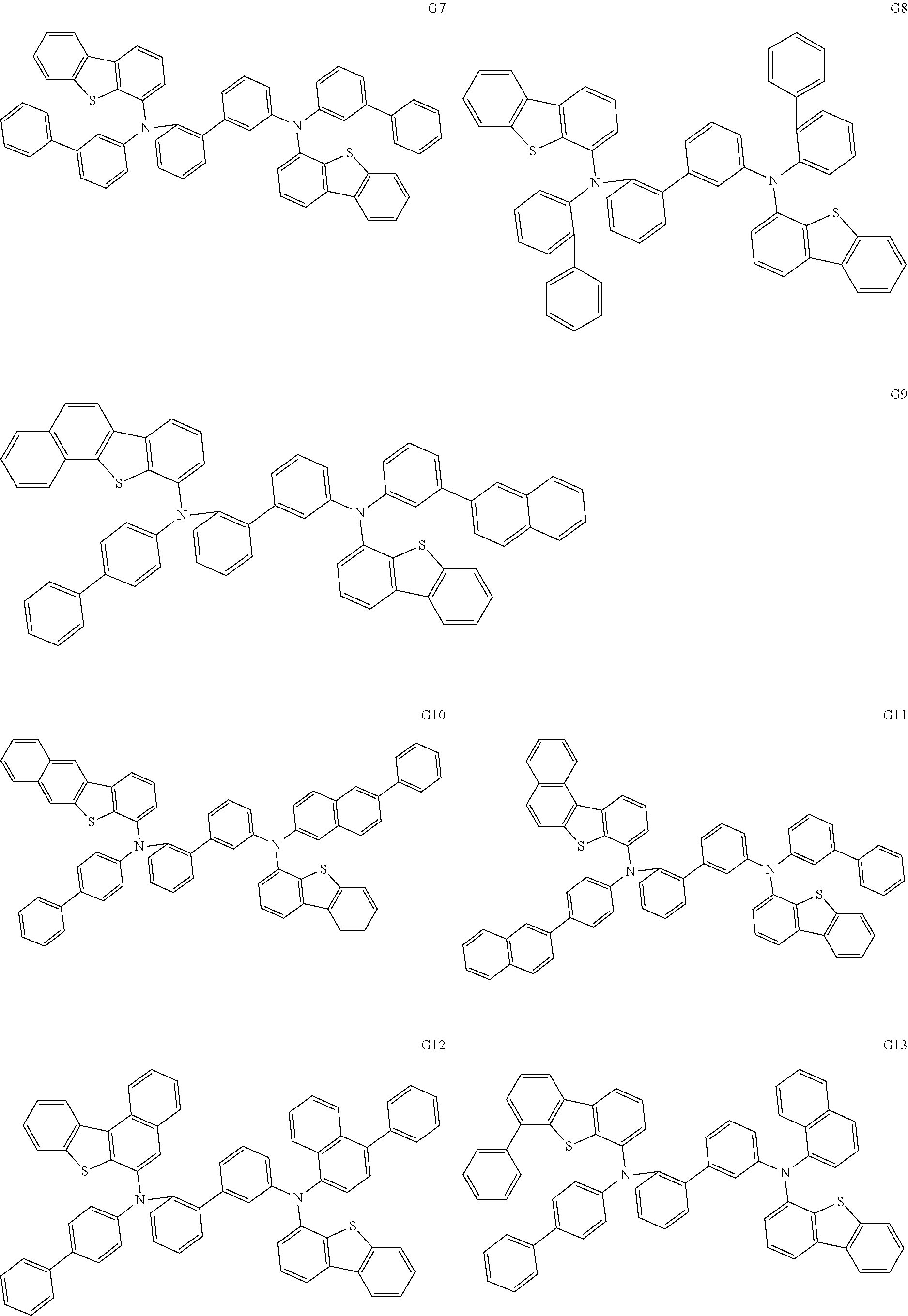

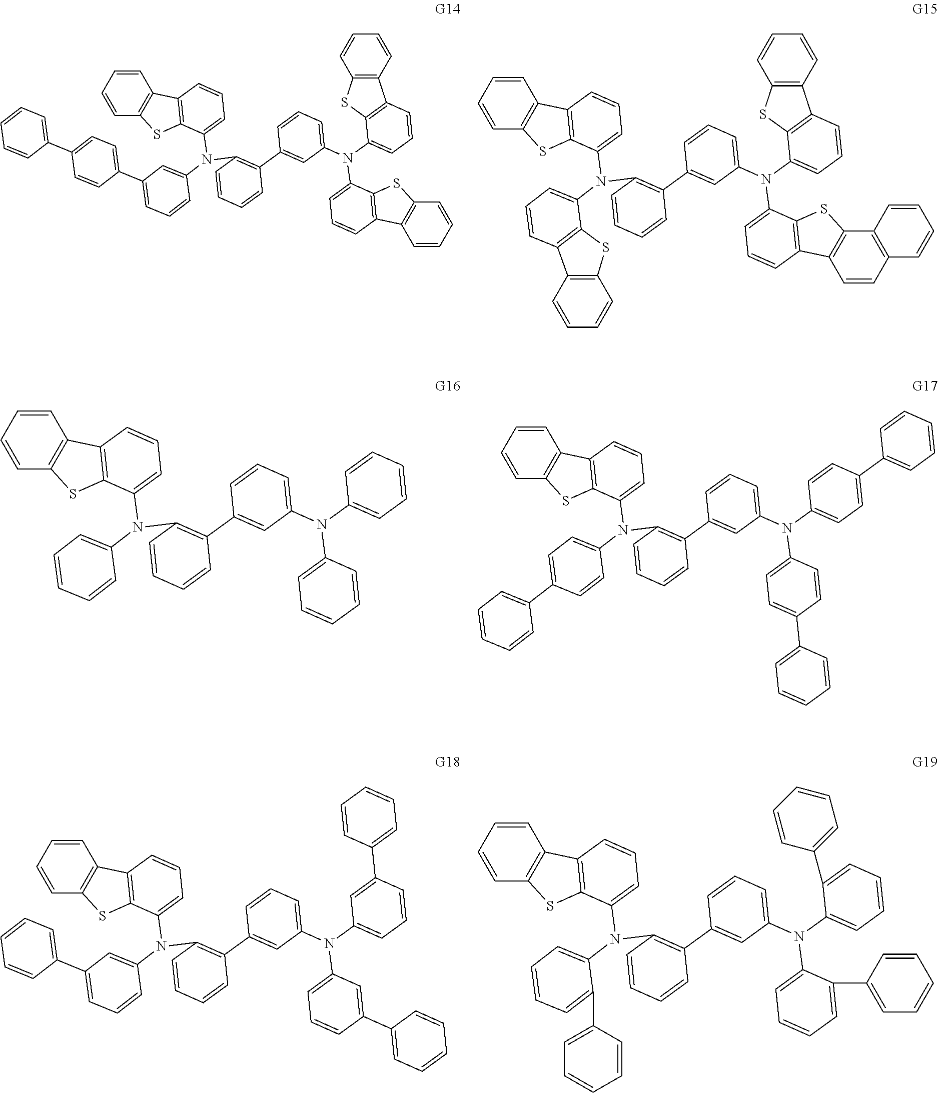

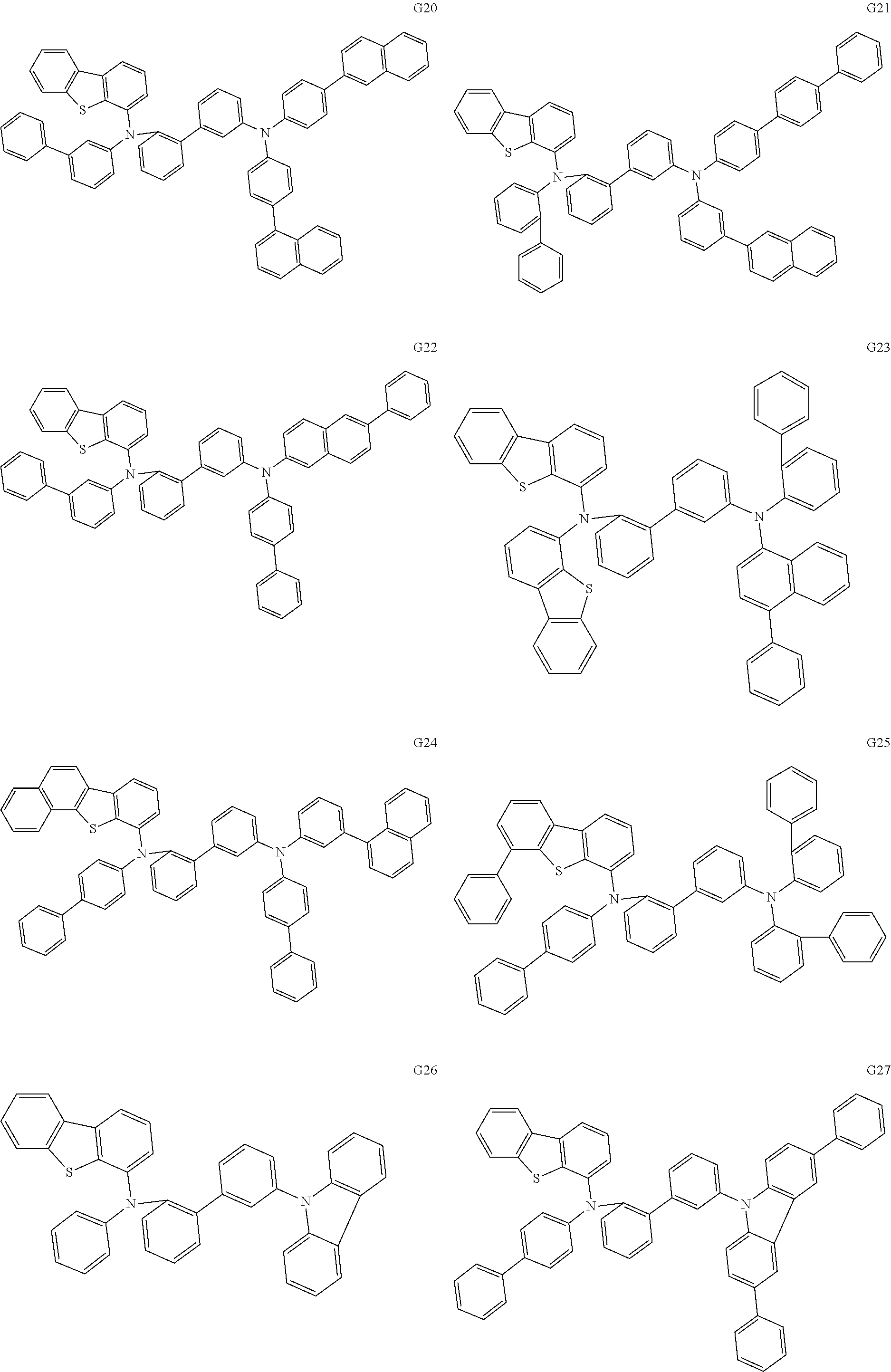

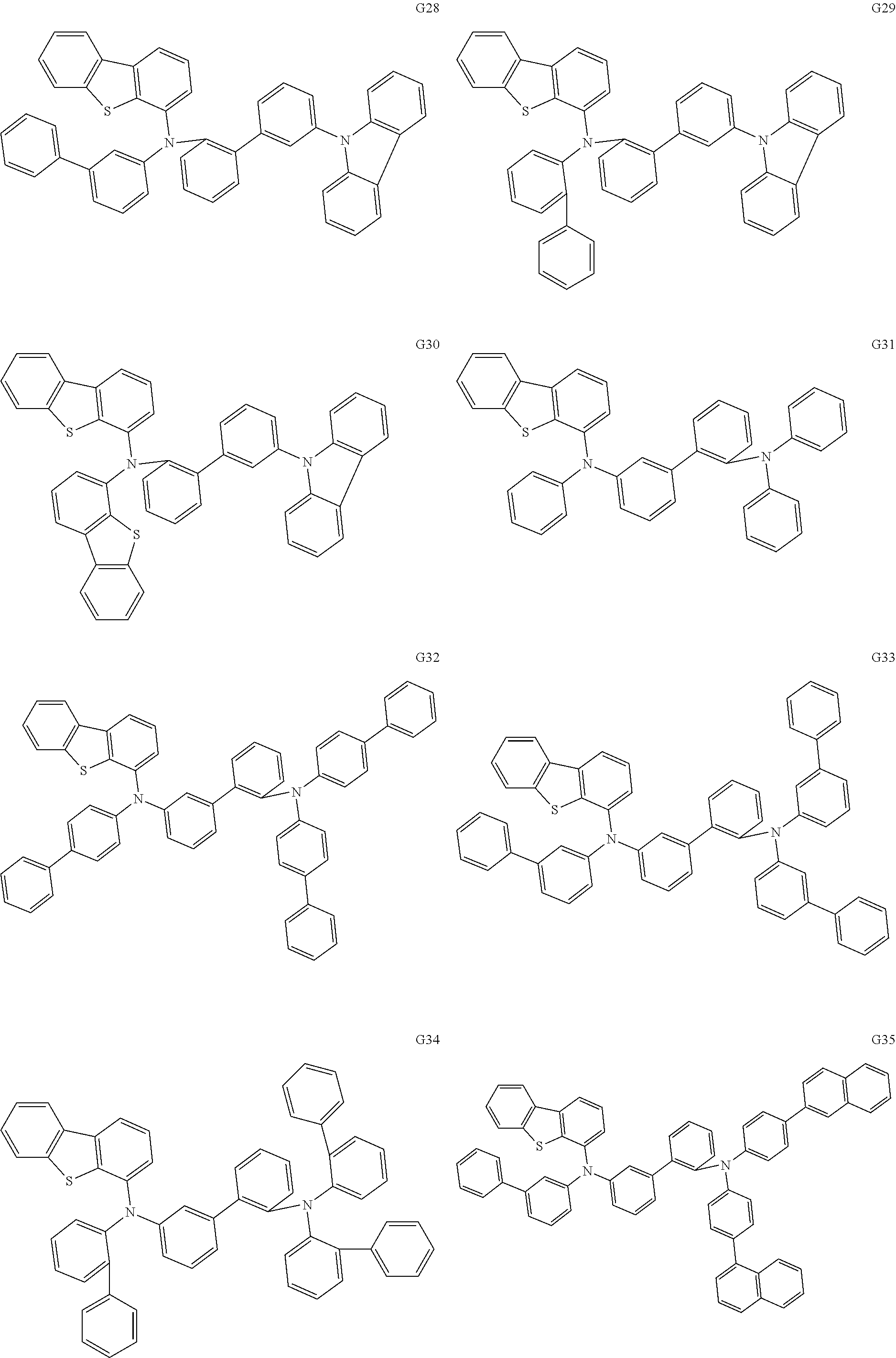

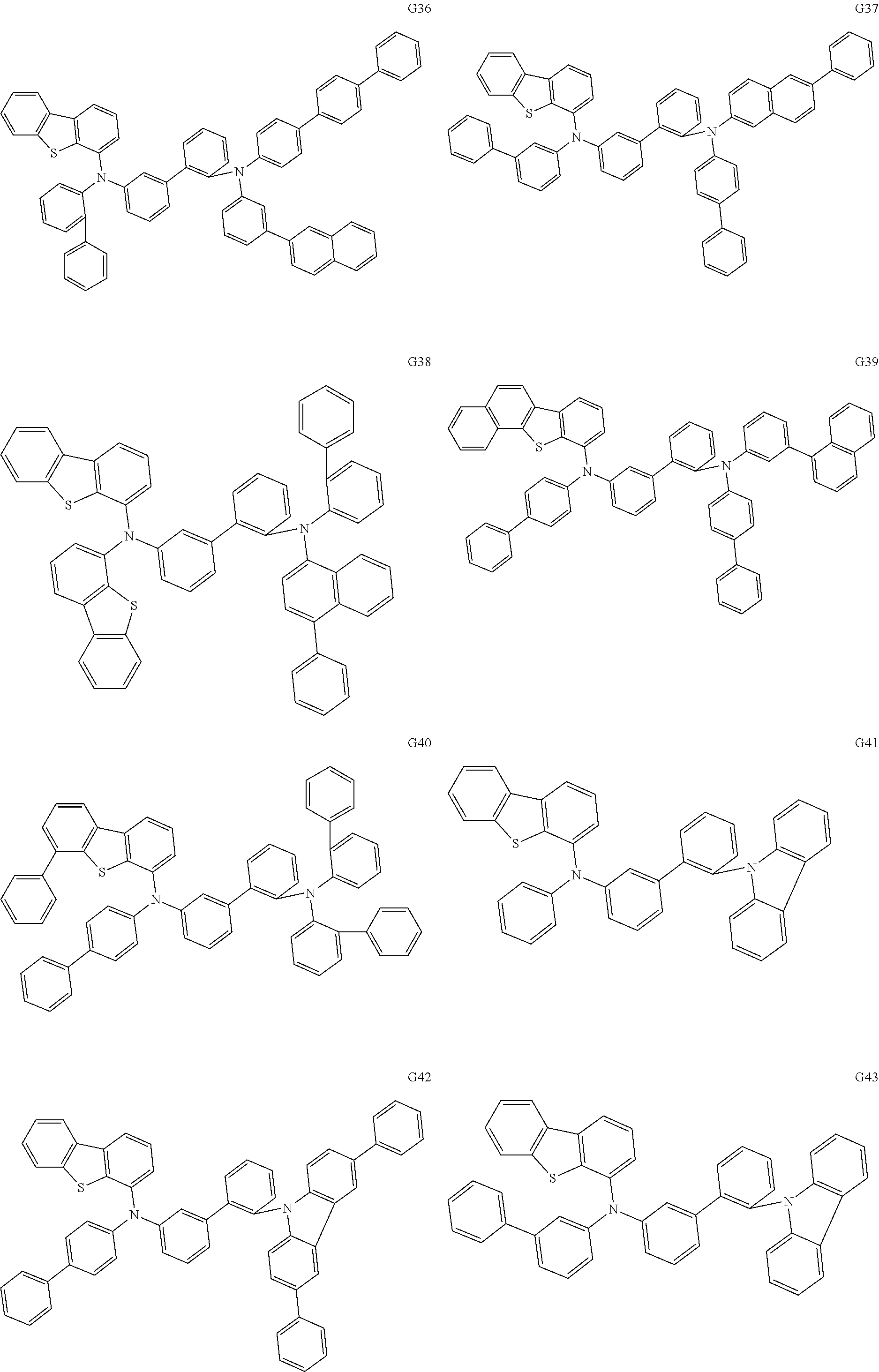

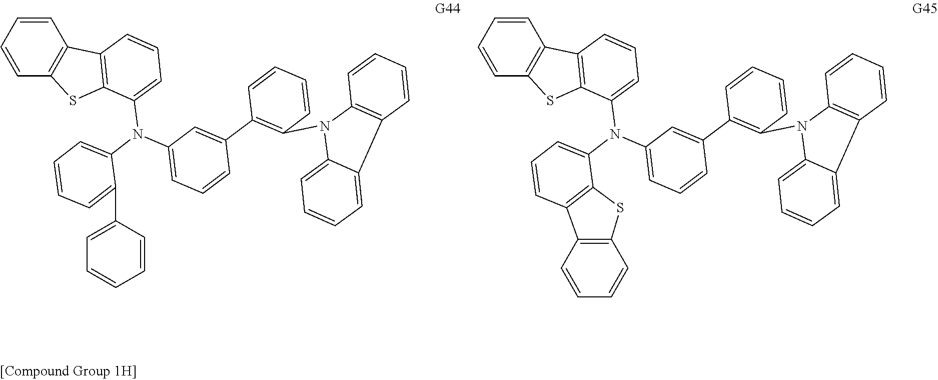 [SMILES: c1ccc(-c2ccccc2N(c2cccc(-c3ccccc3-n3c4ccccc4c4ccccc43)c2)c2cccc3c2sc2ccccc23)cc1 ; c1cc(-c2ccccc2-n2c3ccccc3c3ccccc32)cc(N(c2cccc3c2sc2ccccc23)c2cccc3c2sc2ccccc23)c1]

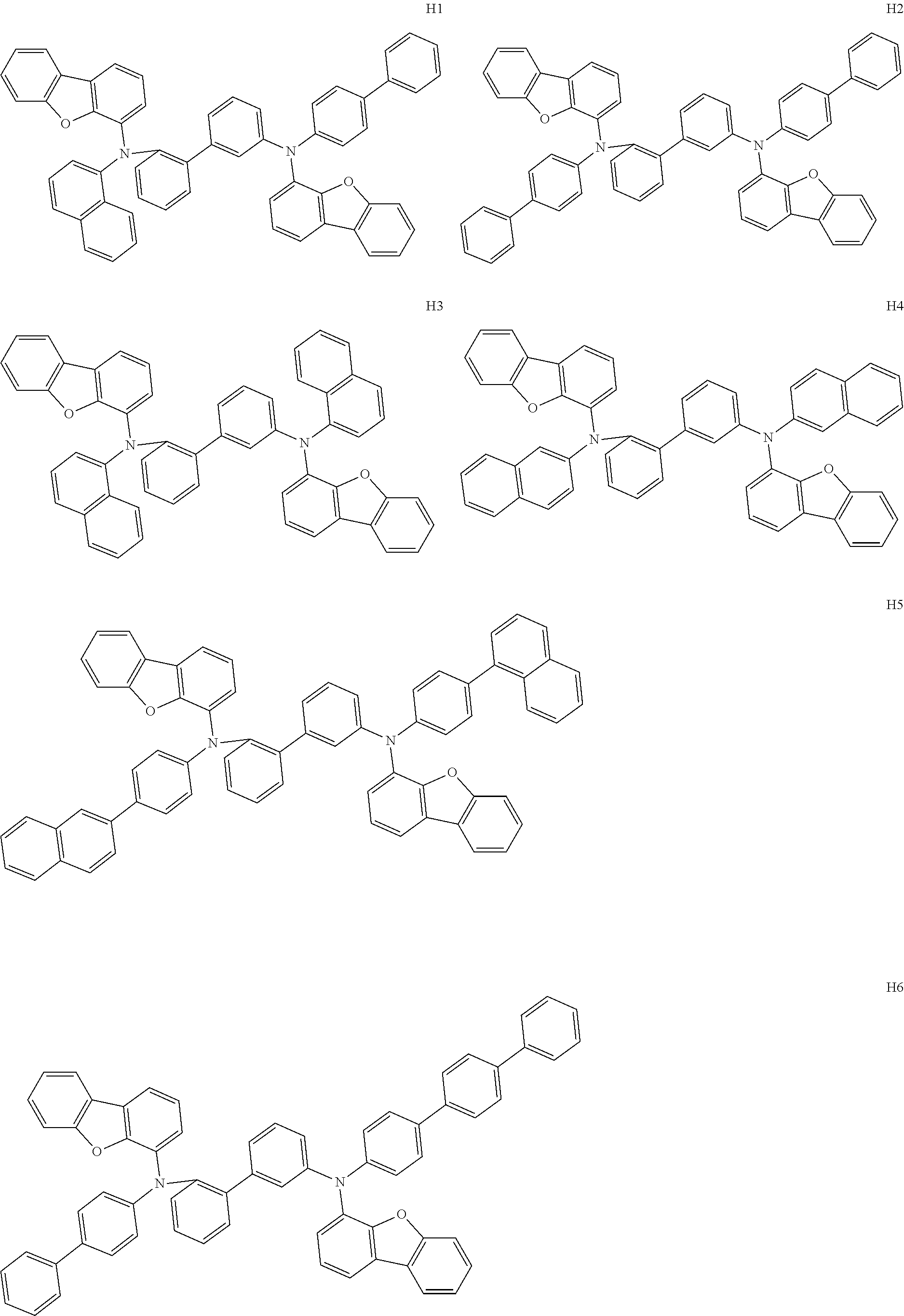

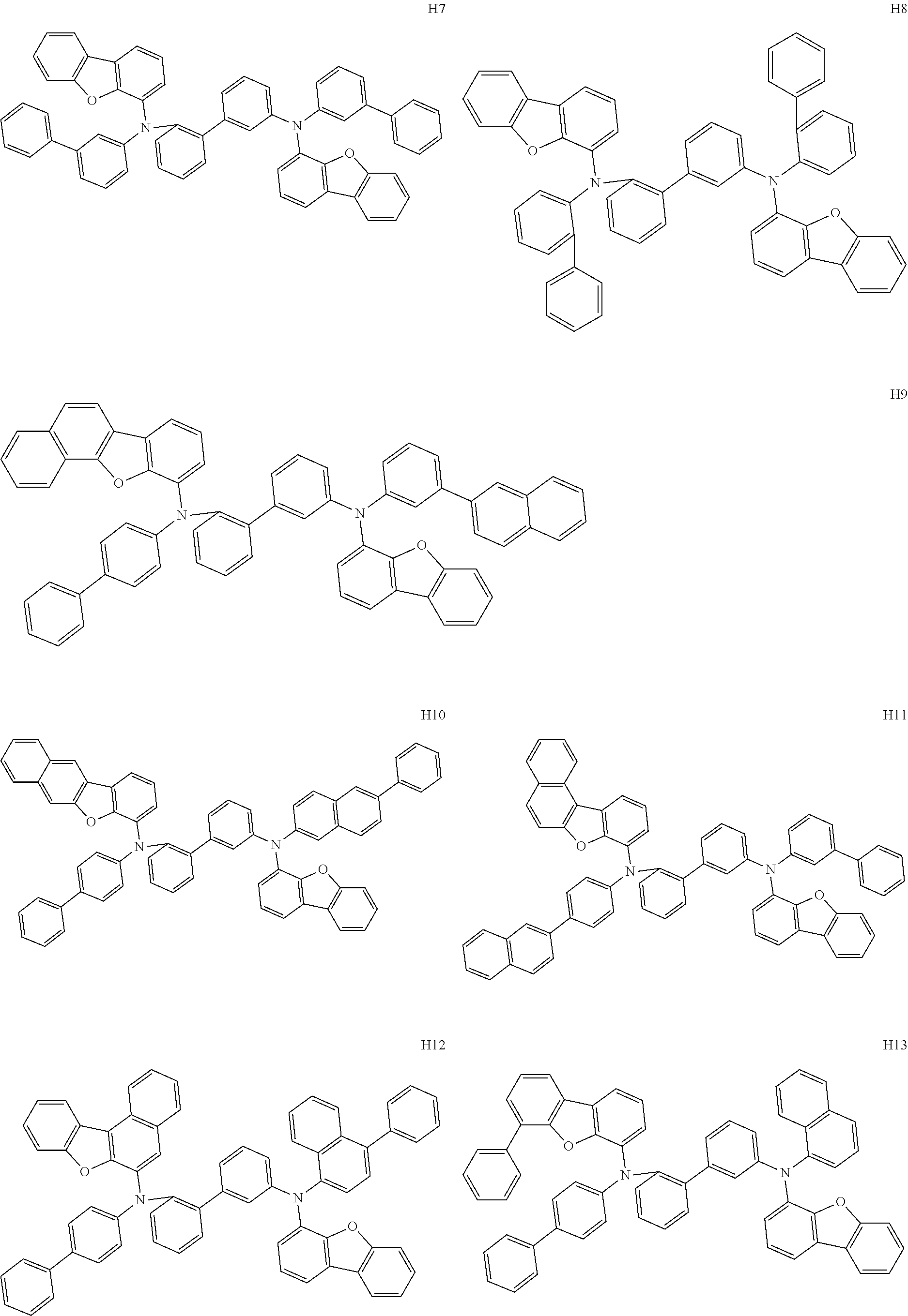

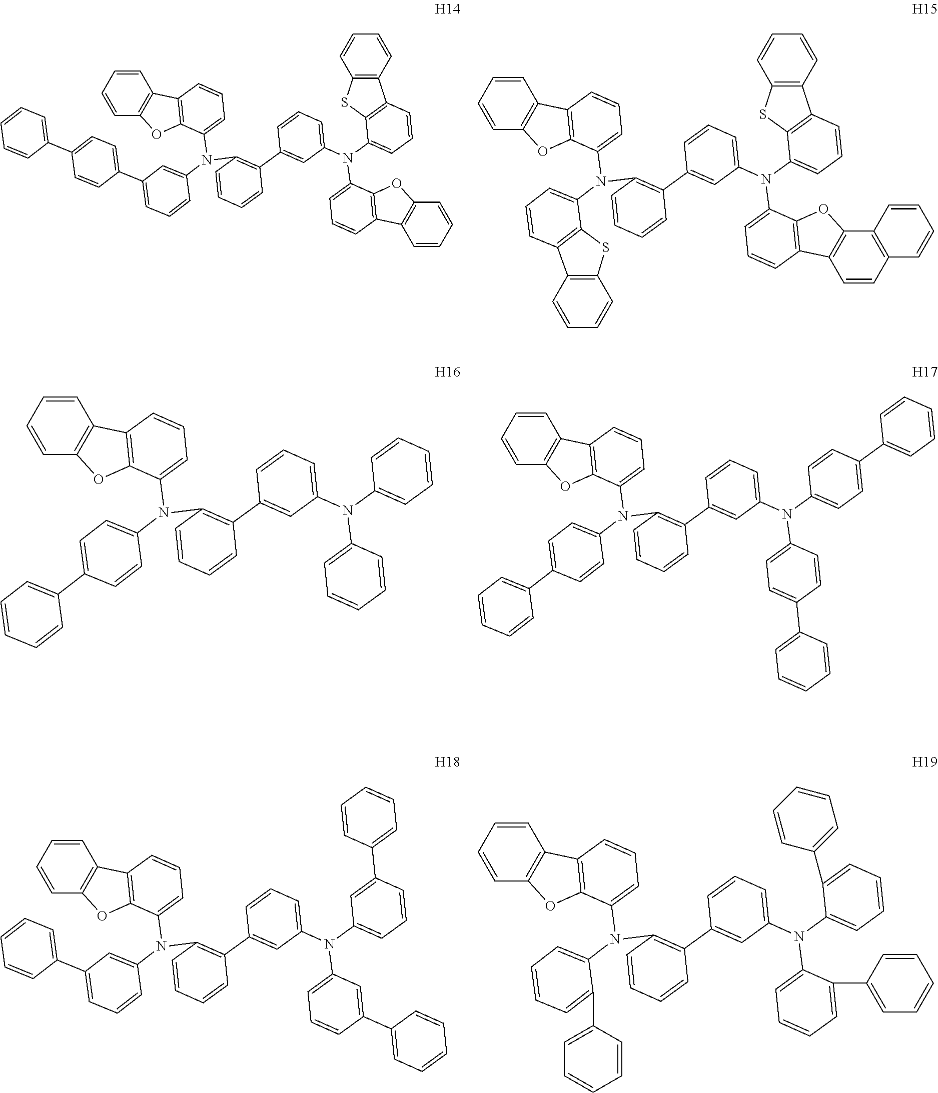

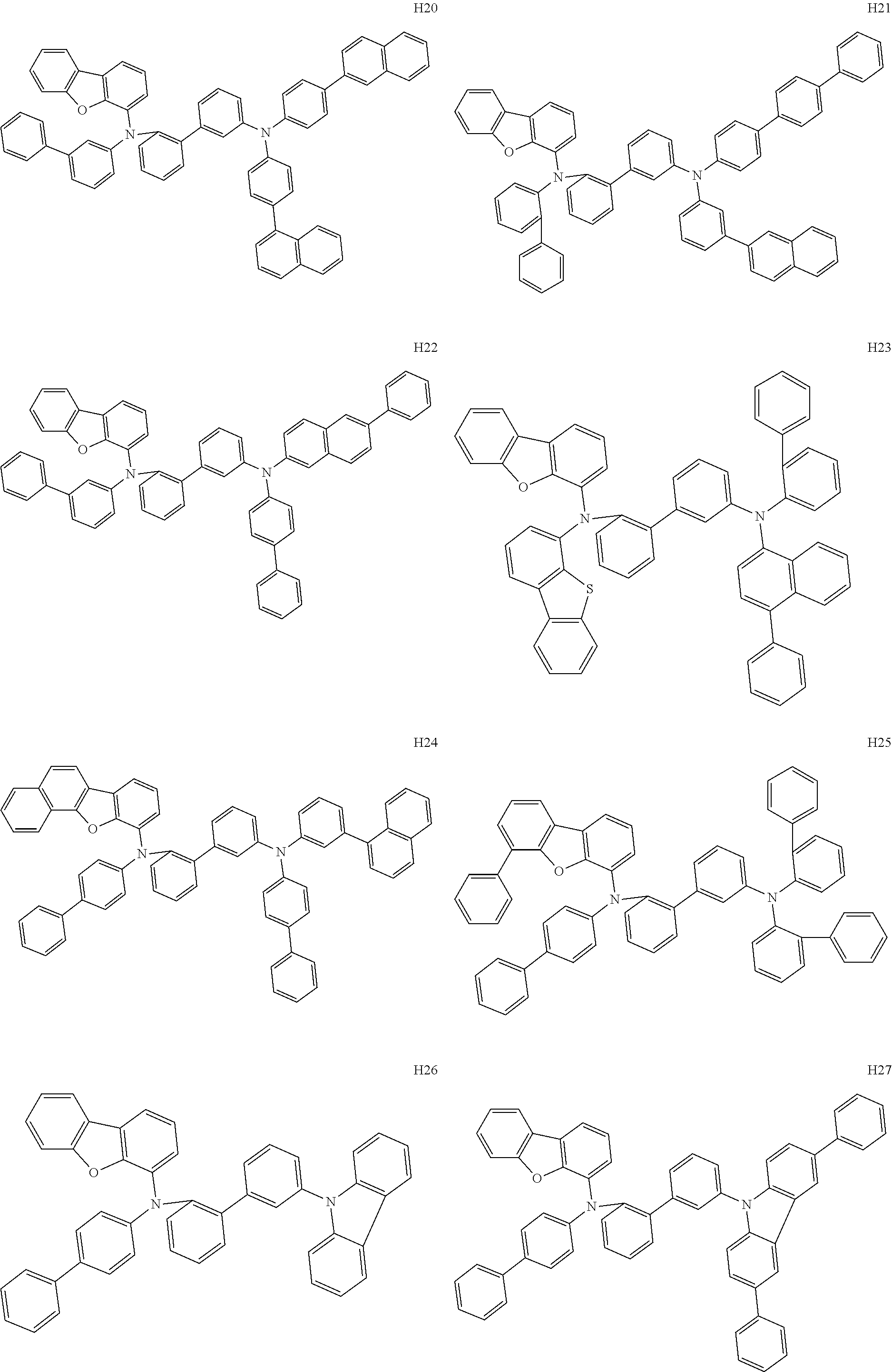

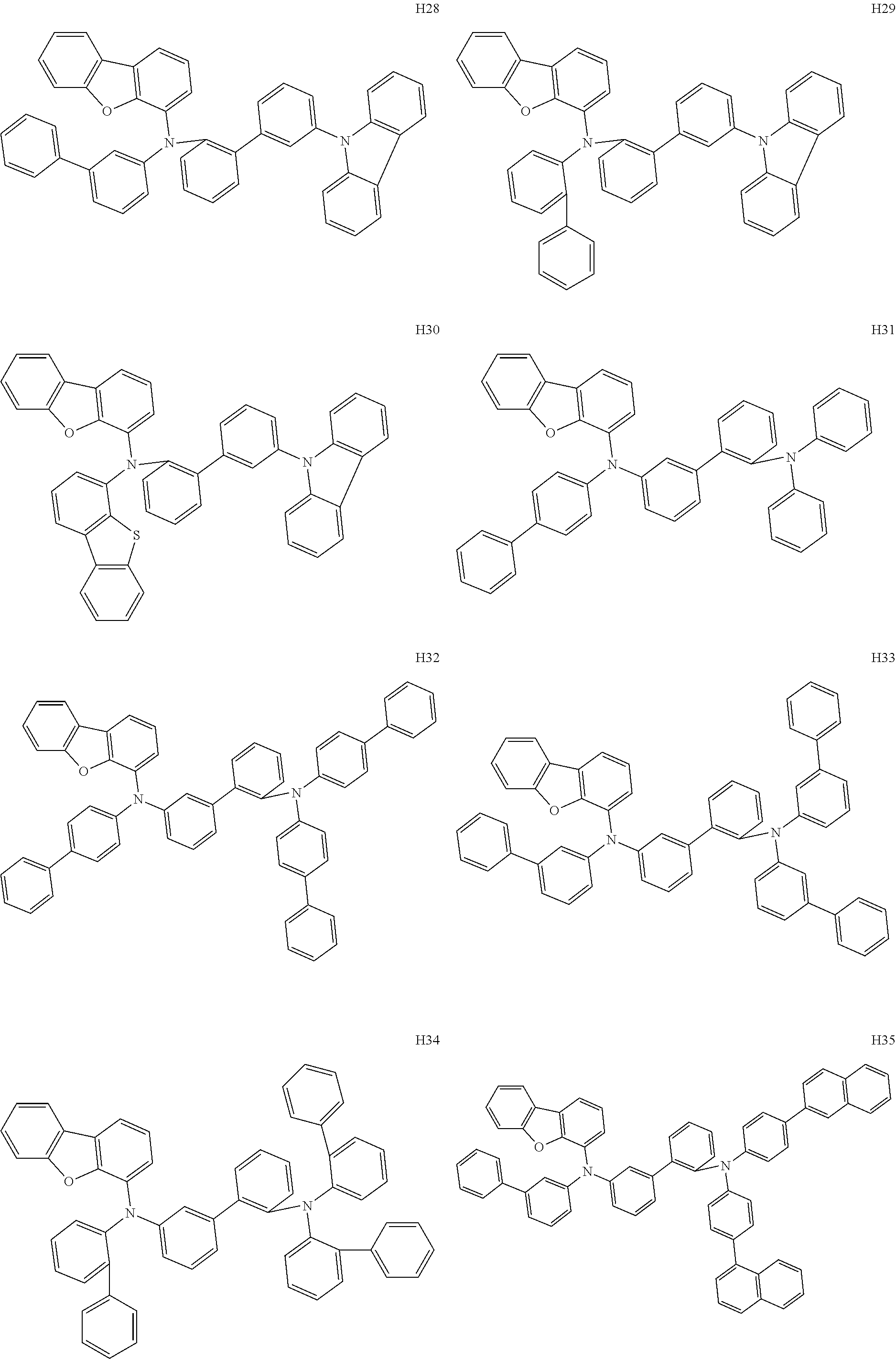

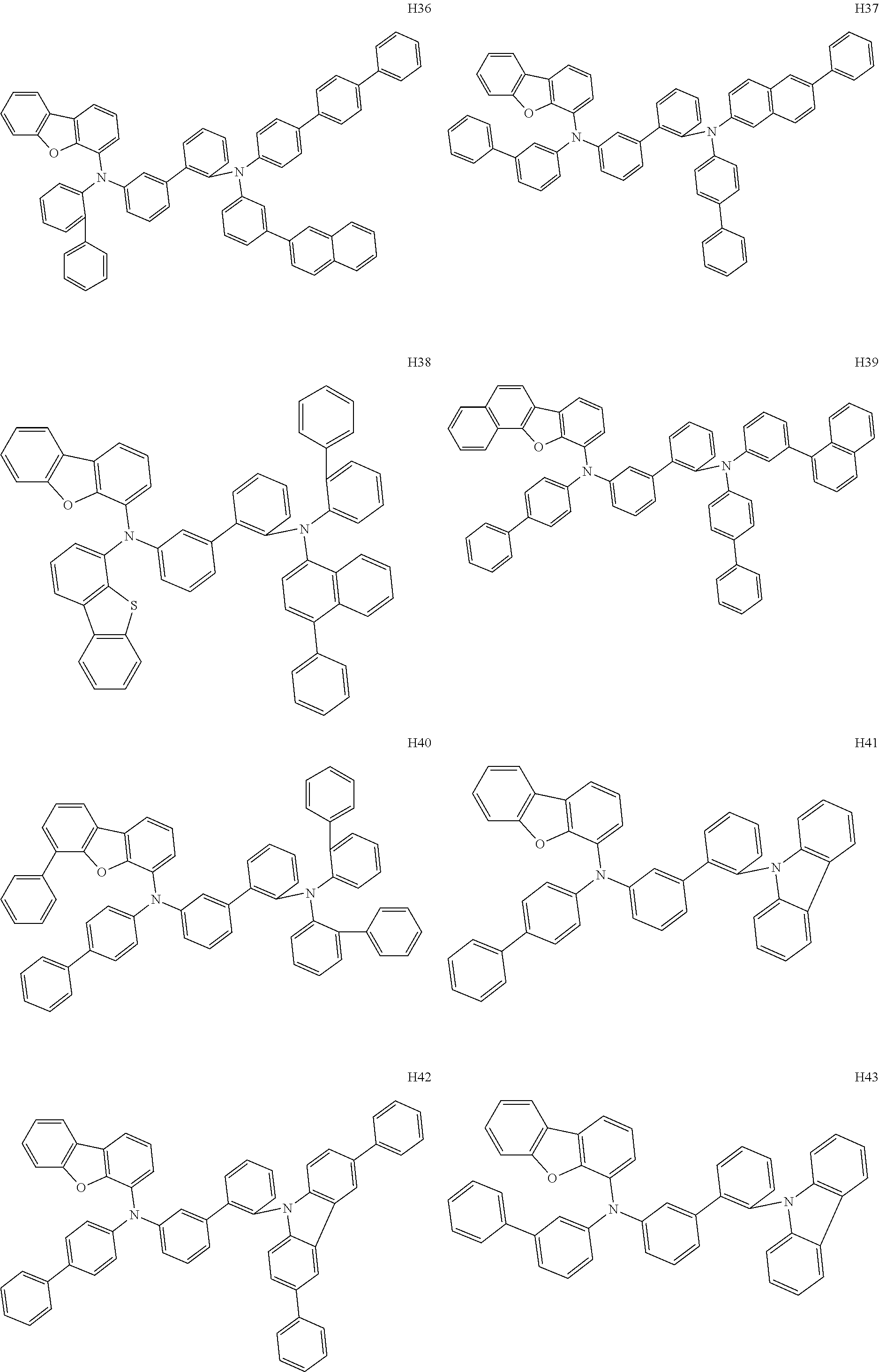

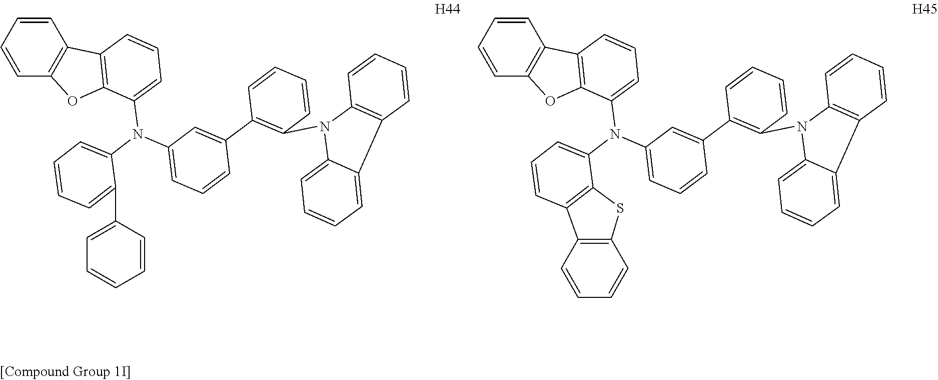 [SMILES: c1ccc(-c2ccccc2N(c2cccc(-c3ccccc3-n3c4ccccc4c4ccccc43)c2)c2cccc3c2oc2ccccc23)cc1 ; c1cc(-c2ccccc2-n2c3ccccc3c3ccccc32)cc(N(c2cccc3c2oc2ccccc23)c2cccc3c2sc2ccccc23)c1]

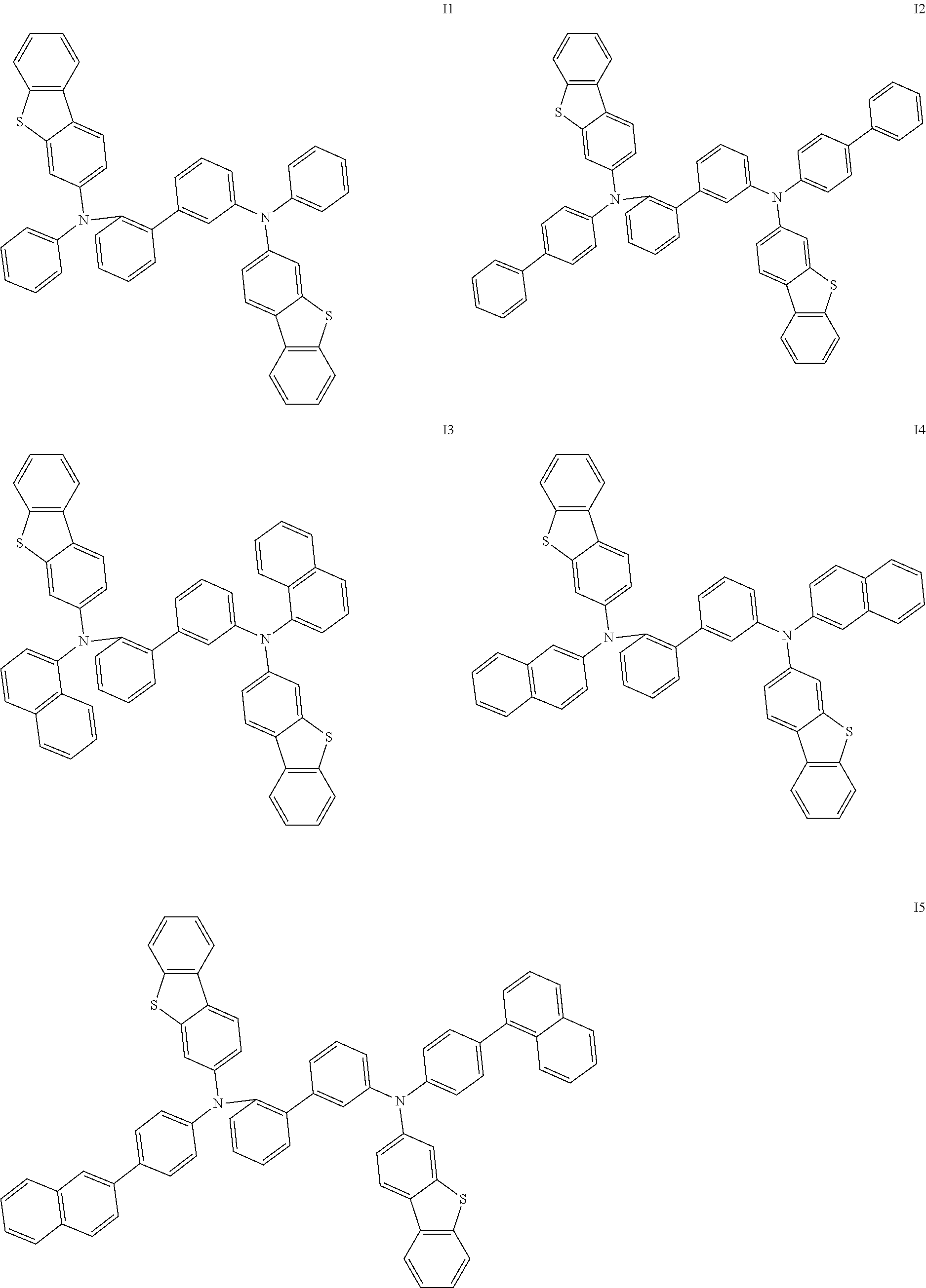

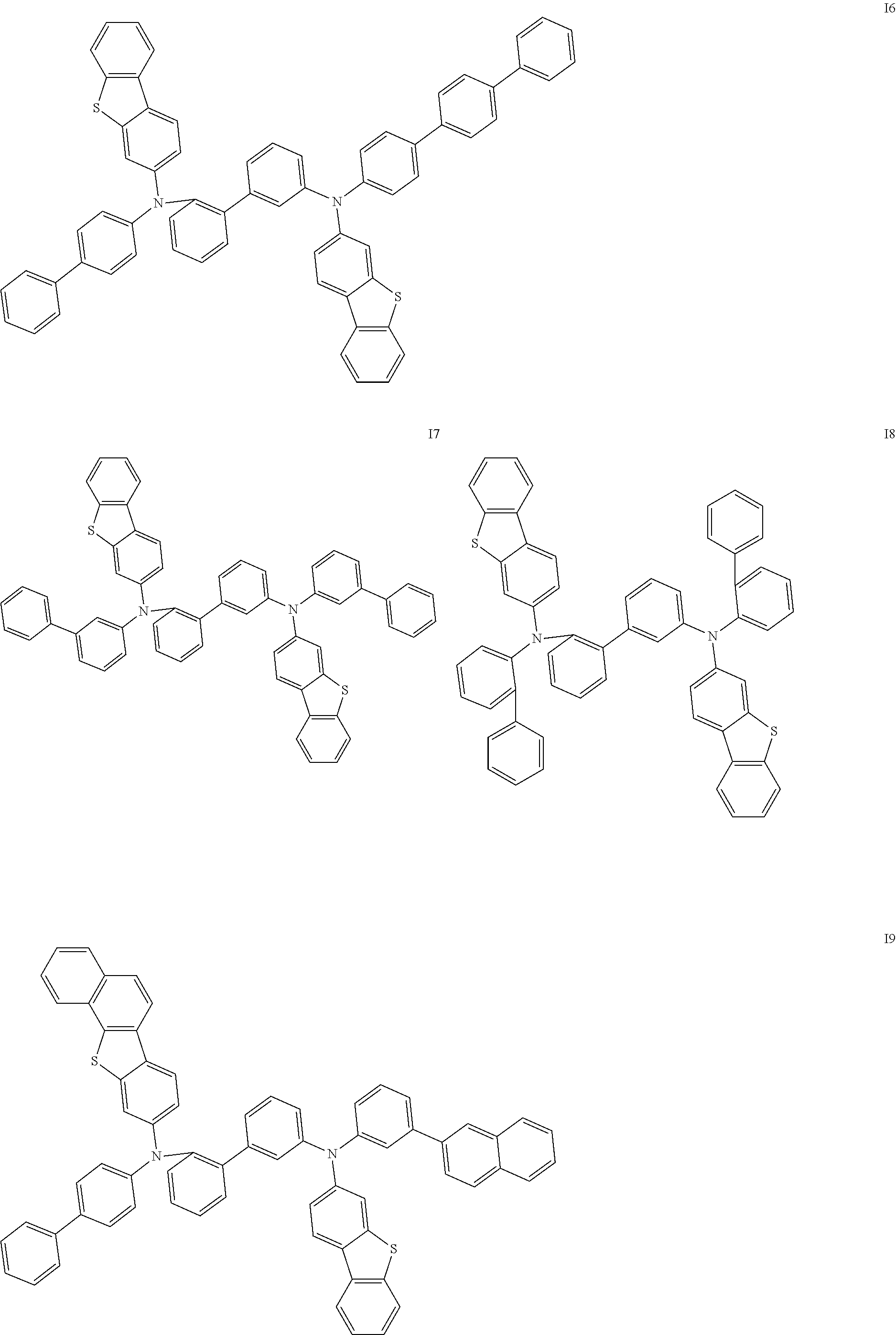 [SMILES: c1ccc(-c2ccc(-c3ccc(N(c4cccc(-c5ccccc5N(c5ccc(-c6ccccc6)cc5)c5ccc6c(c5)sc5ccccc56)c4)c4ccc5c(c4)sc4ccccc45)cc3)cc2)cc1 ; c1ccc(-c2cccc(N(c3cccc(-c4ccccc4N(c4cccc(-c5ccccc5)c4)c4ccc5c(c4)sc4ccccc45)c3)c3ccc4c(c3)sc3ccccc34)c2)cc1 ; c1ccc(-c2ccccc2N(c2cccc(-c3ccccc3N(c3ccc4c(c3)sc3ccccc34)c3ccccc3-c3ccccc3)c2)c2ccc3c(c2)sc2ccccc23)cc1 ; c1ccc(-c2ccc(N(c3ccc4c(c3)sc3c5ccccc5ccc43)c3ccccc3-c3cccc(N(c4cccc(-c5ccc6ccccc6c5)c4)c4ccc5c(c4)sc4ccccc45)c3)cc2)cc1]

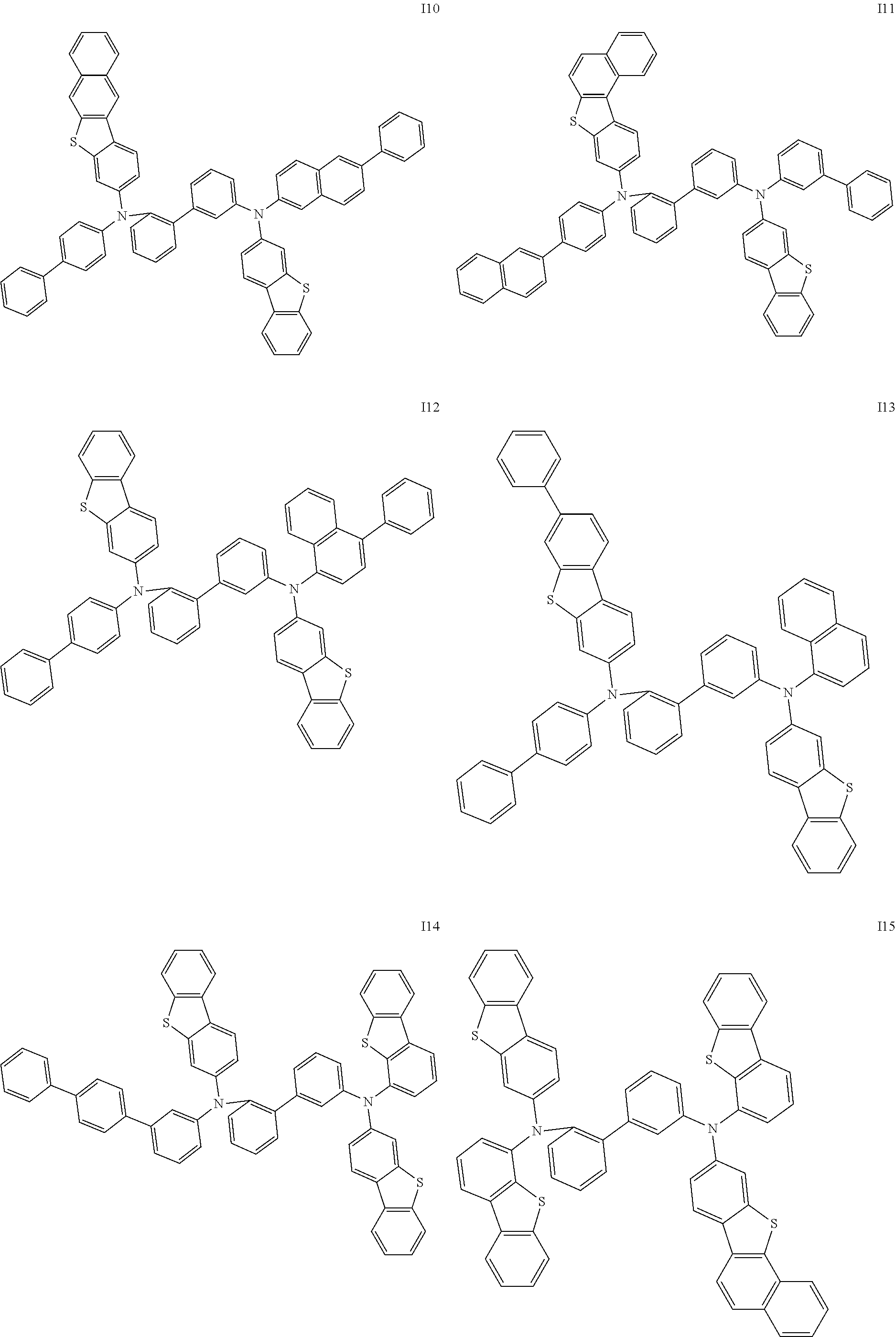

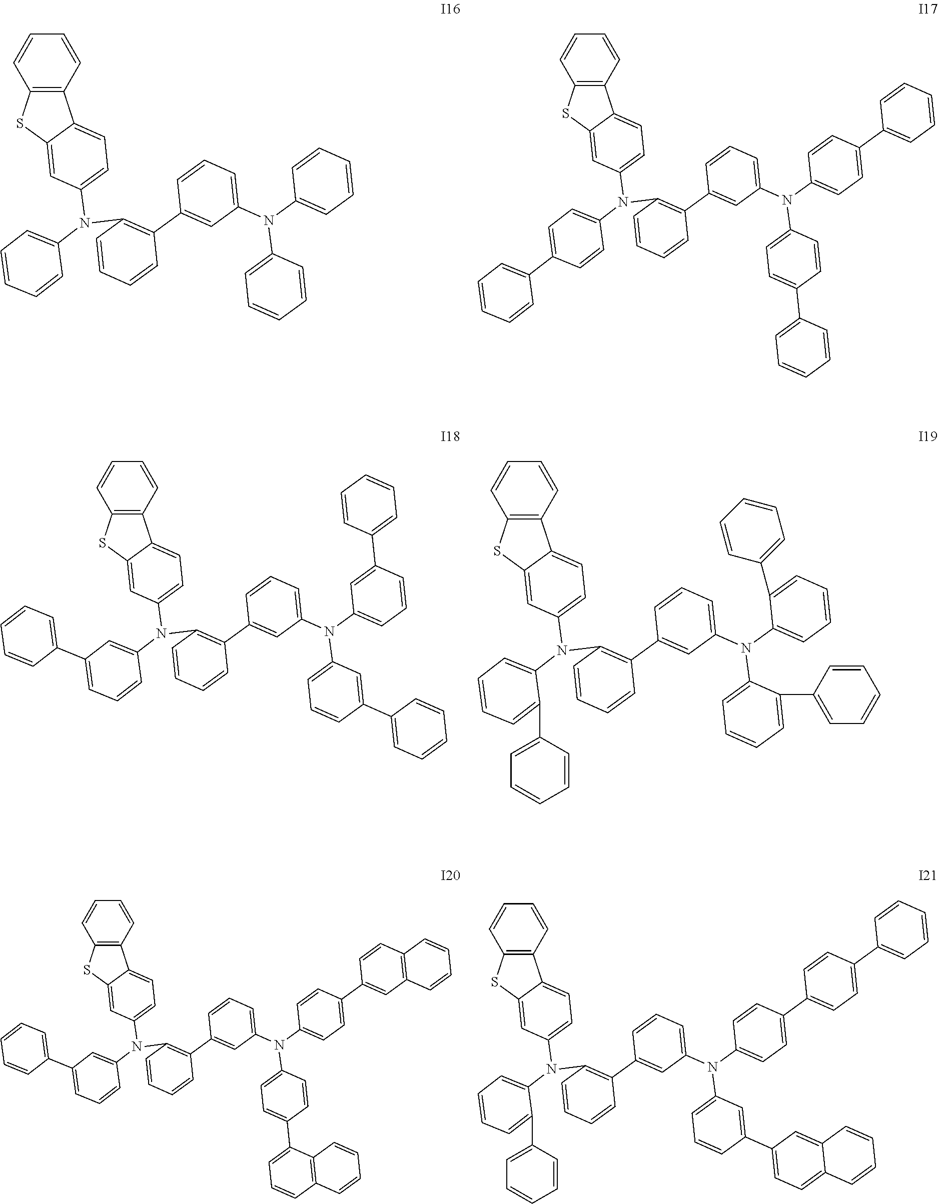

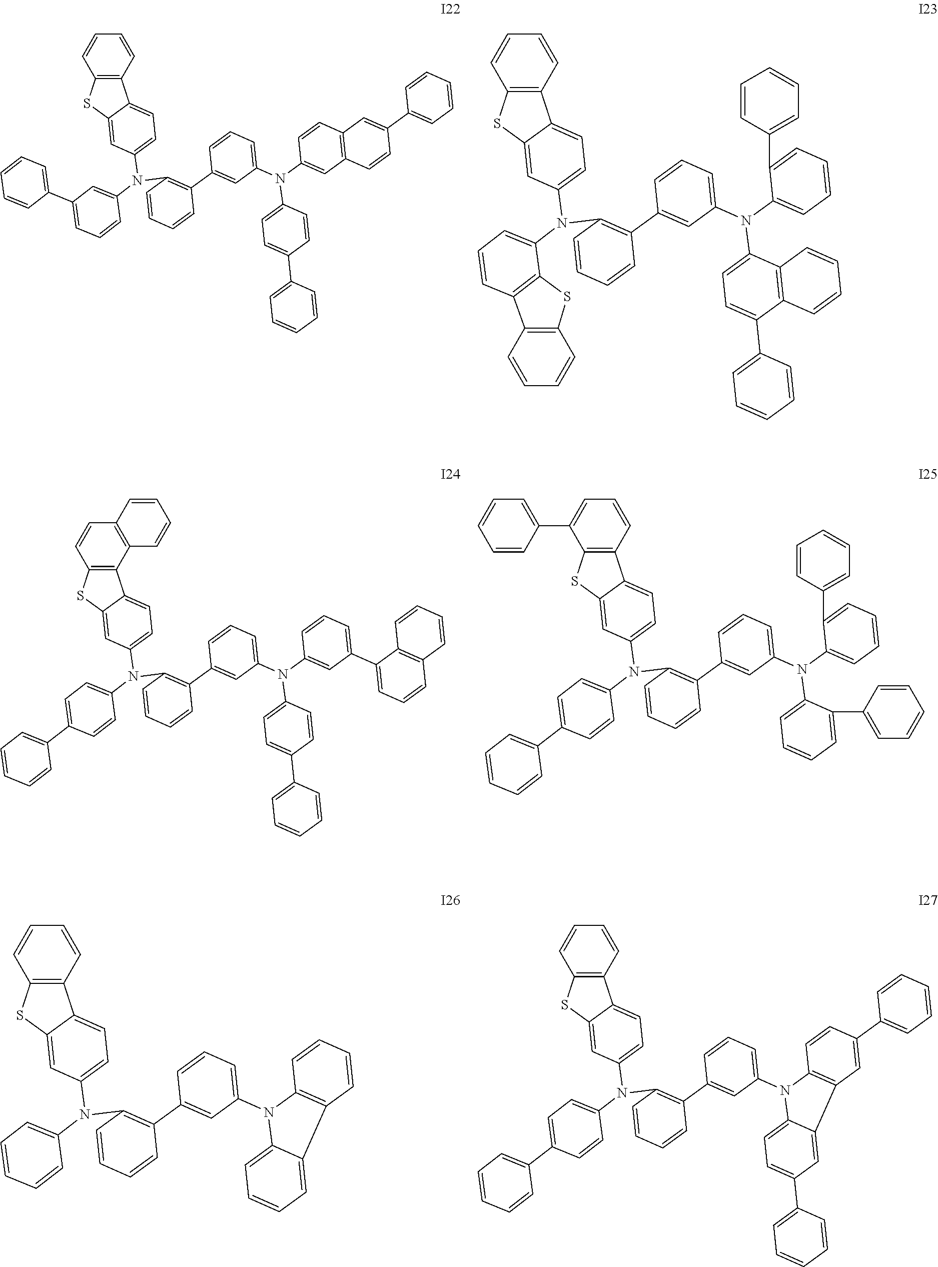

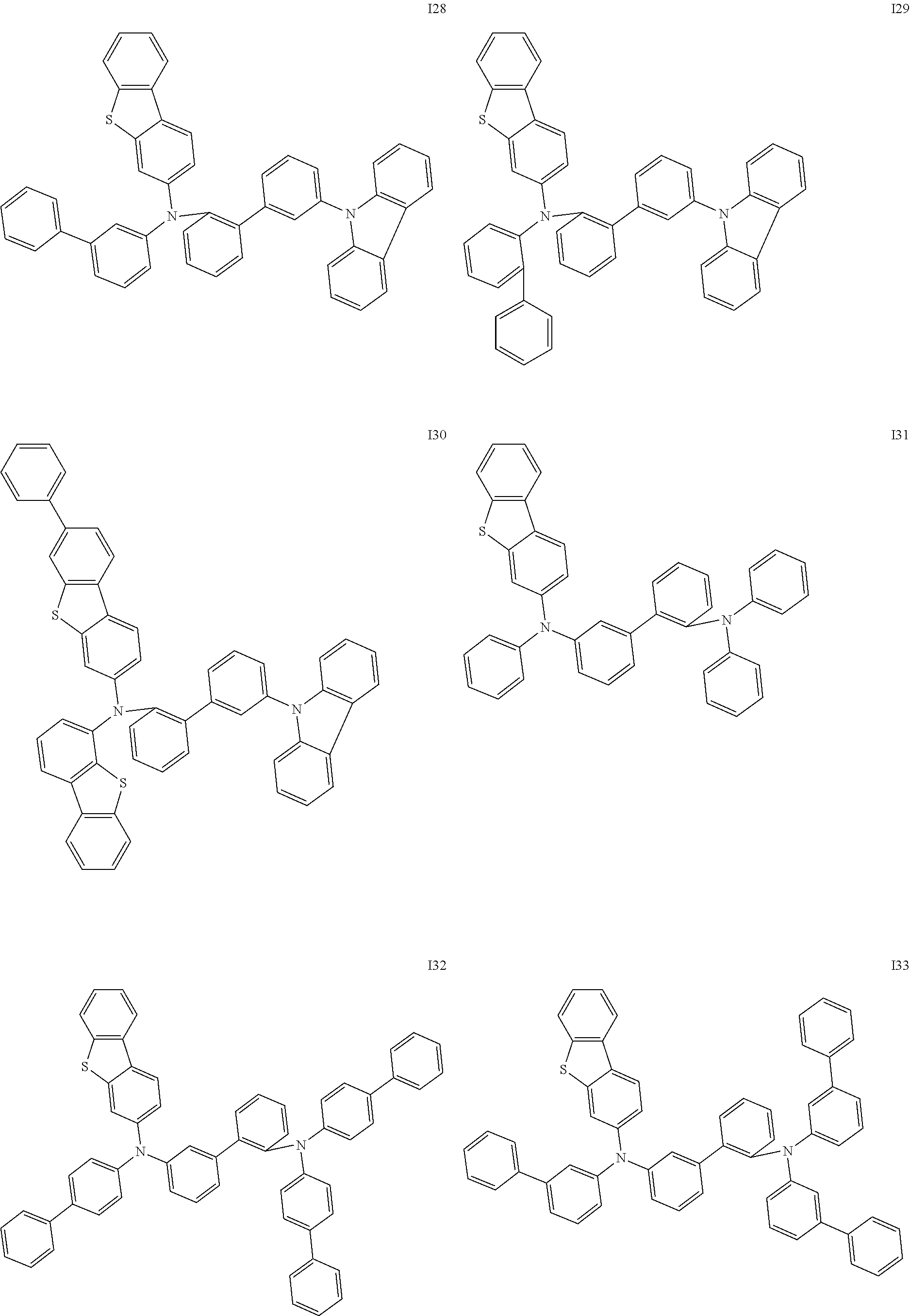

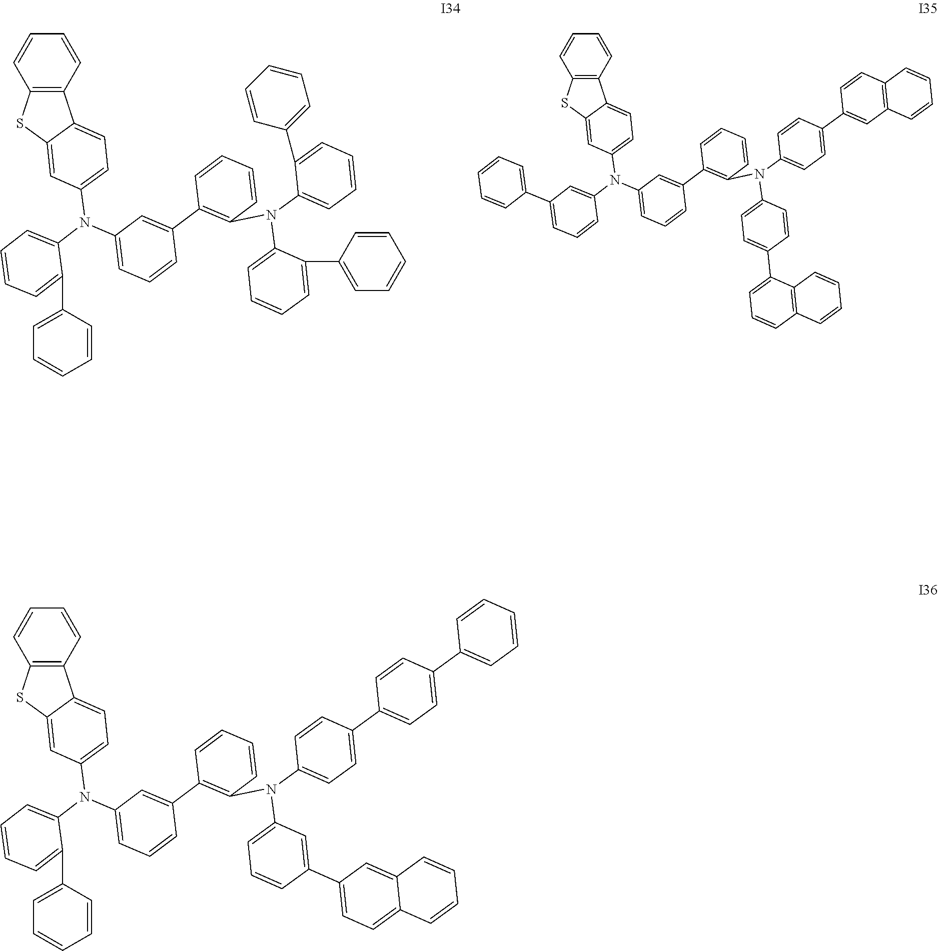 [SMILES: c1ccc(-c2ccccc2N(c2cccc(-c3ccccc3N(c3ccccc3-c3ccccc3)c3ccccc3-c3ccccc3)c2)c2ccc3c(c2)sc2ccccc23)cc1 ; c1ccc(-c2cccc(N(c3cccc(-c4ccccc4N(c4ccc(-c5ccc6ccccc6c5)cc4)c4ccc(-c5cccc6ccccc56)cc4)c3)c3ccc4c(c3)sc3ccccc34)c2)cc1 ; c1ccc(-c2ccc(-c3ccc(N(c4cccc(-c5ccc6ccccc6c5)c4)c4ccccc4-c4cccc(N(c5ccc6c(c5)sc5ccccc56)c5ccccc5-c5ccccc5)c4)cc3)cc2)cc1]

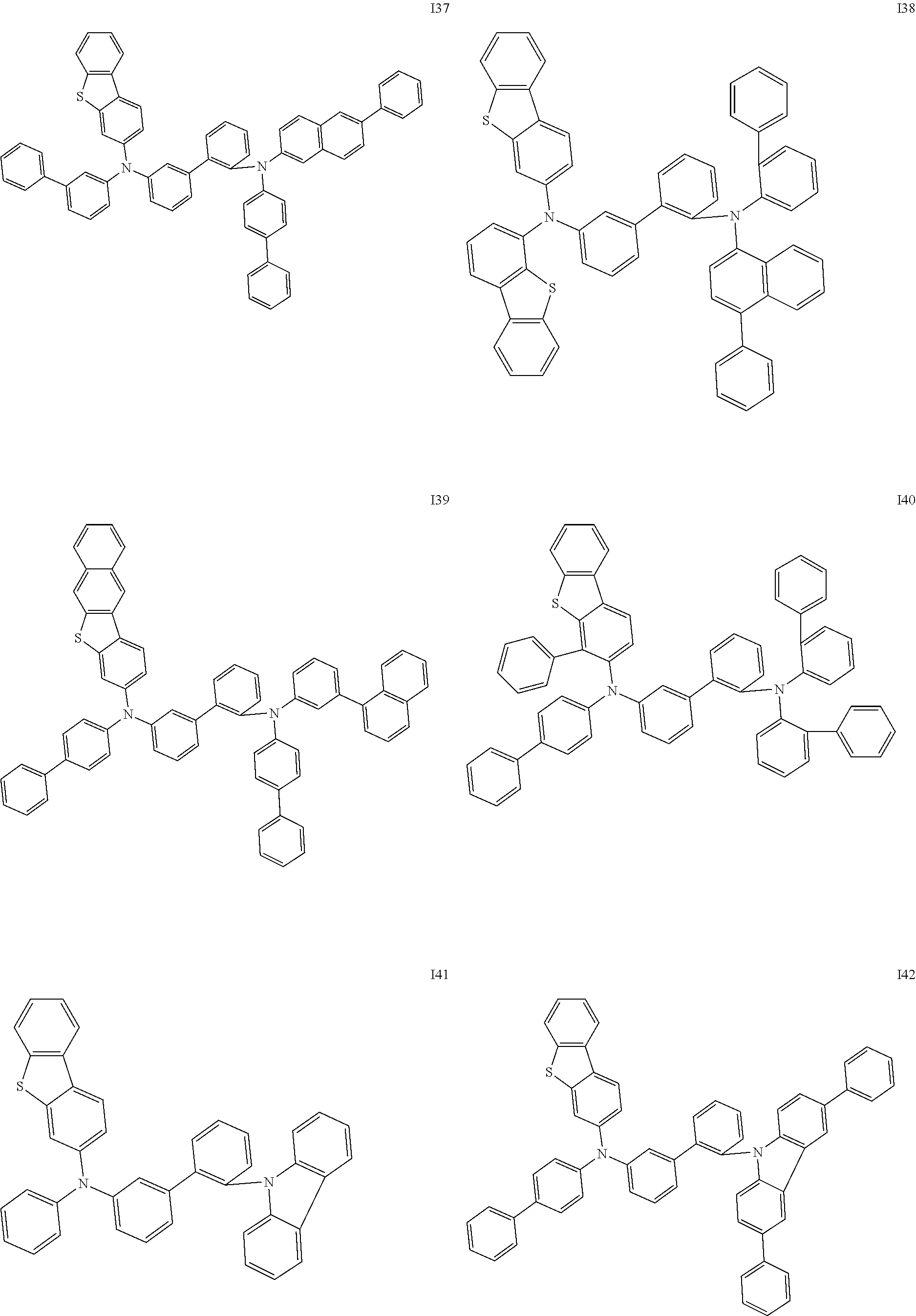

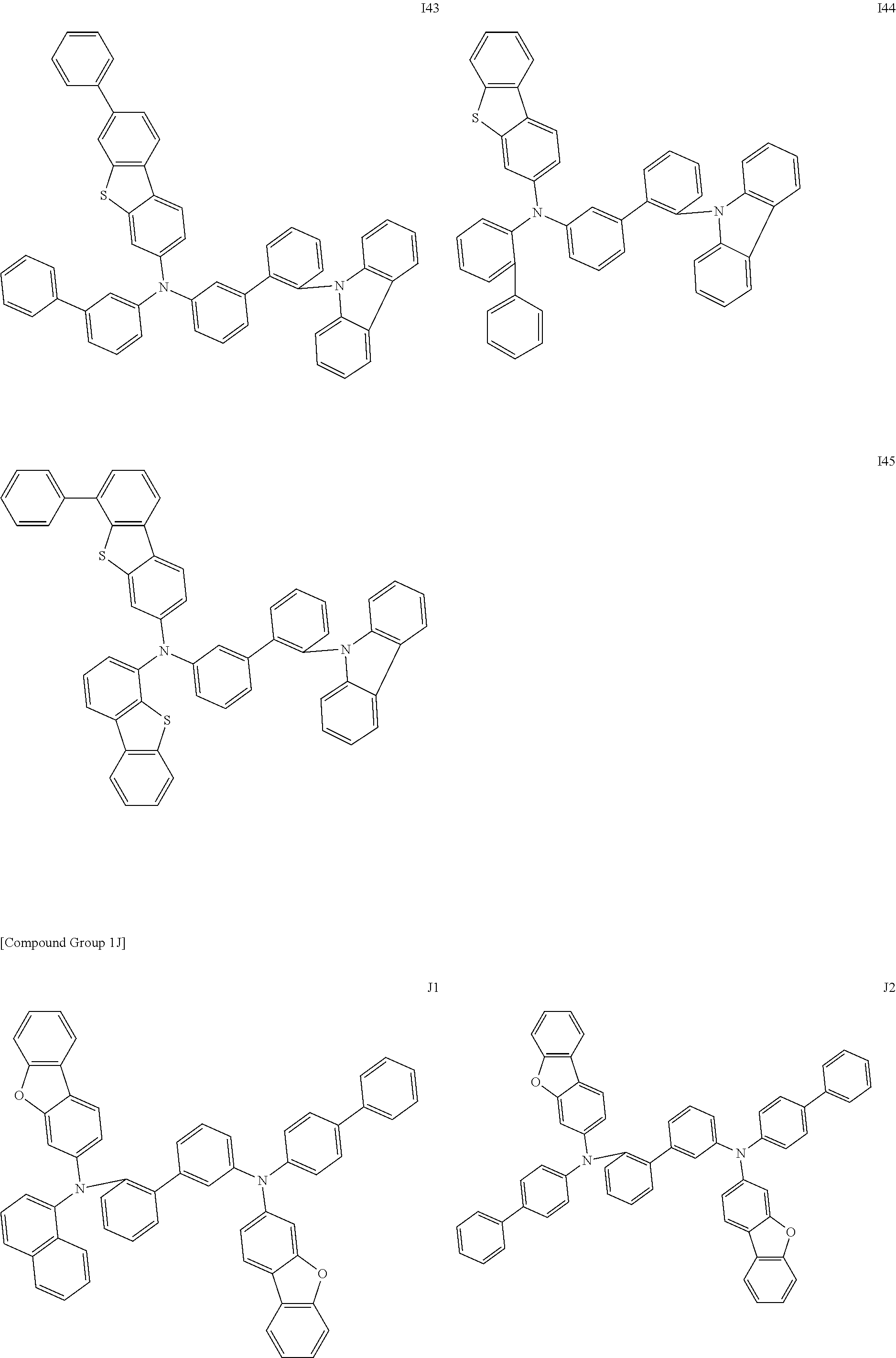

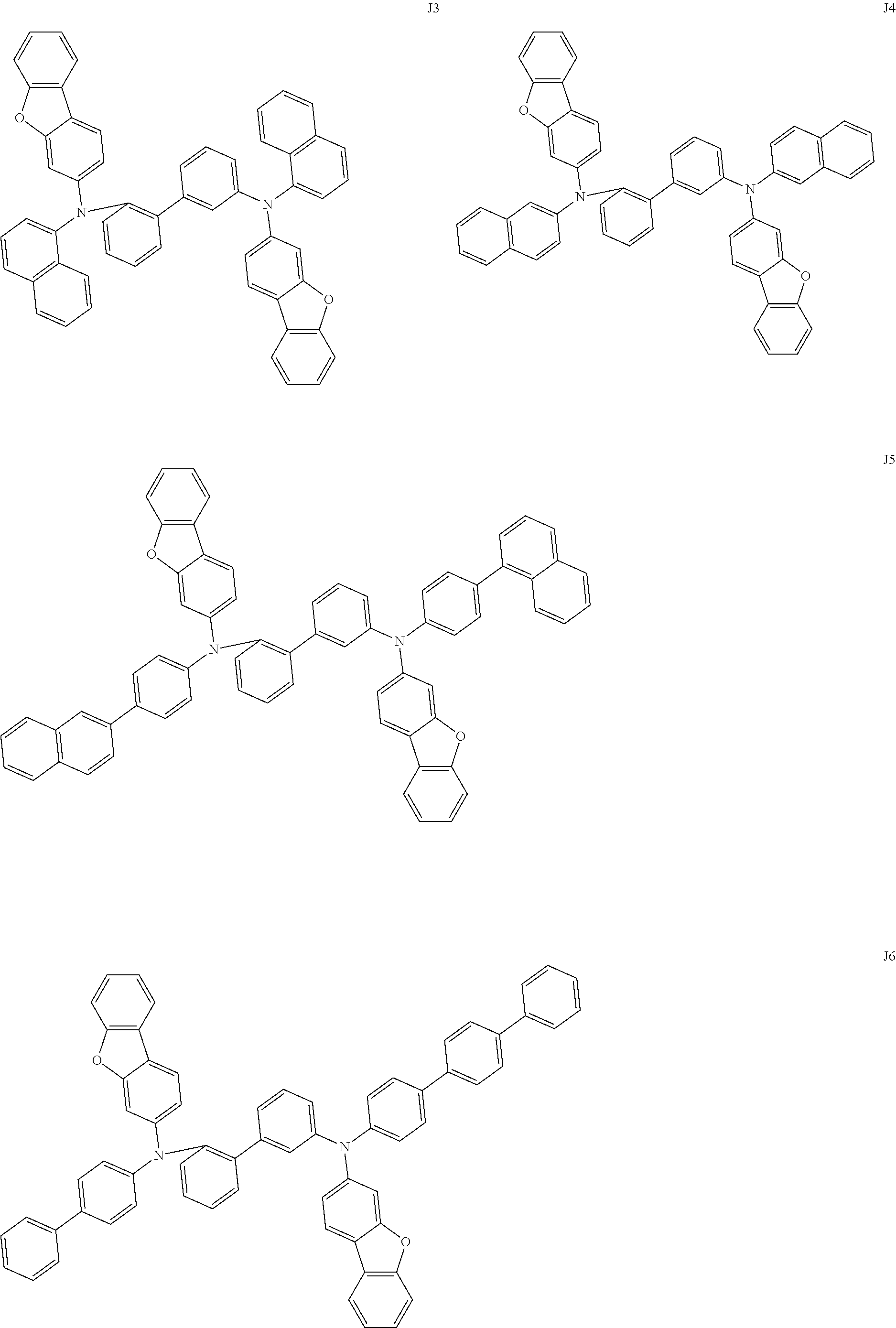 [SMILES: c1cc(-c2ccccc2N(c2ccc3c(c2)oc2ccccc23)c2cccc3ccccc23)cc(N(c2ccc3c(c2)oc2ccccc23)c2cccc3ccccc23)c1 ; c1cc(-c2ccccc2N(c2ccc3ccccc3c2)c2ccc3c(c2)oc2ccccc23)cc(N(c2ccc3ccccc3c2)c2ccc3c(c2)oc2ccccc23)c1 ; c1cc(-c2ccccc2N(c2ccc(-c3ccc4ccccc4c3)cc2)c2ccc3c(c2)oc2ccccc23)cc(N(c2ccc(-c3cccc4ccccc34)cc2)c2ccc3c(c2)oc2ccccc23)c1 ; c1ccc(-c2ccc(-c3ccc(N(c4cccc(-c5ccccc5N(c5ccc(-c6ccccc6)cc5)c5ccc6c(c5)oc5ccccc56)c4)c4ccc5c(c4)oc4ccccc45)cc3)cc2)cc1]

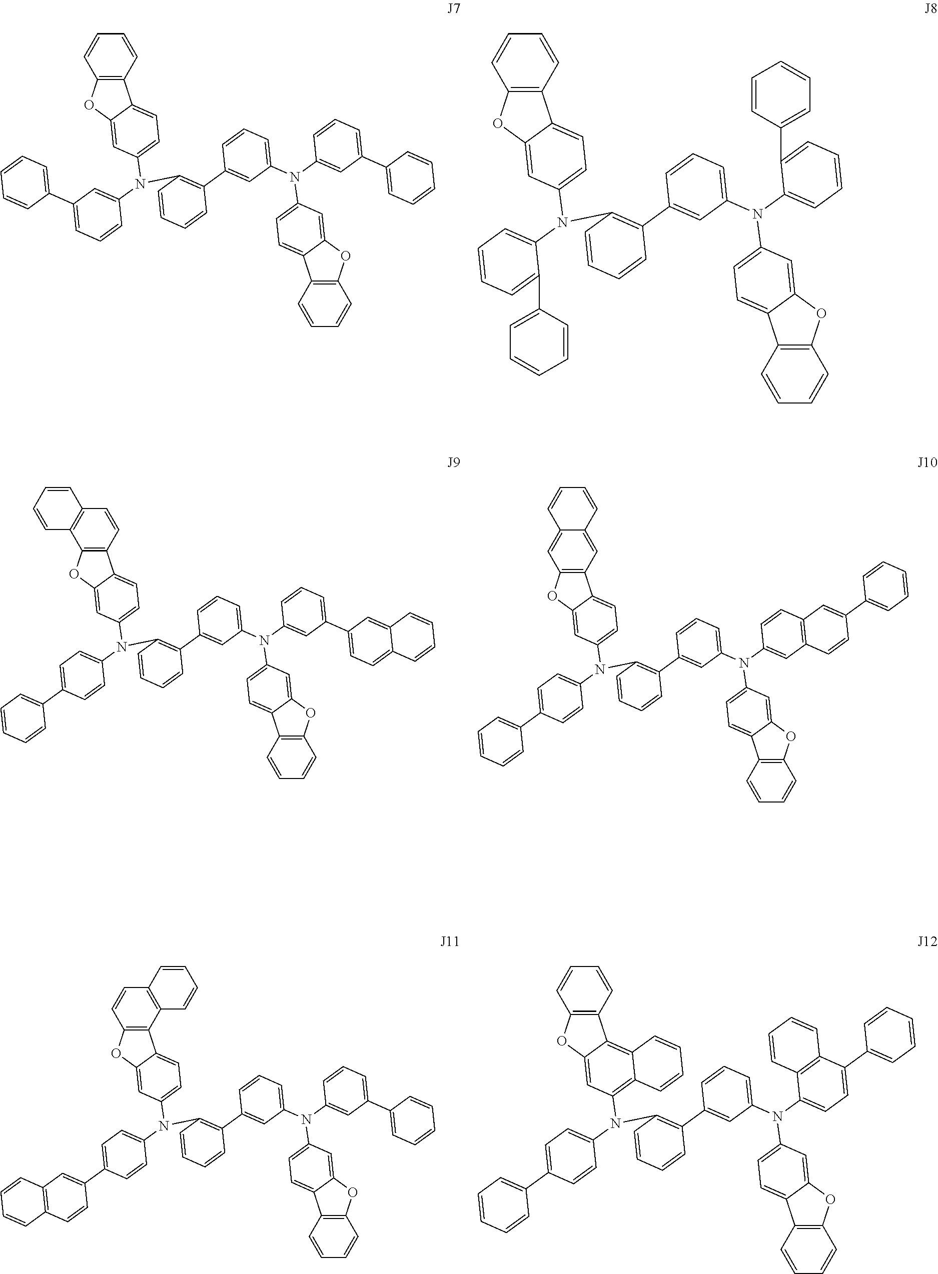

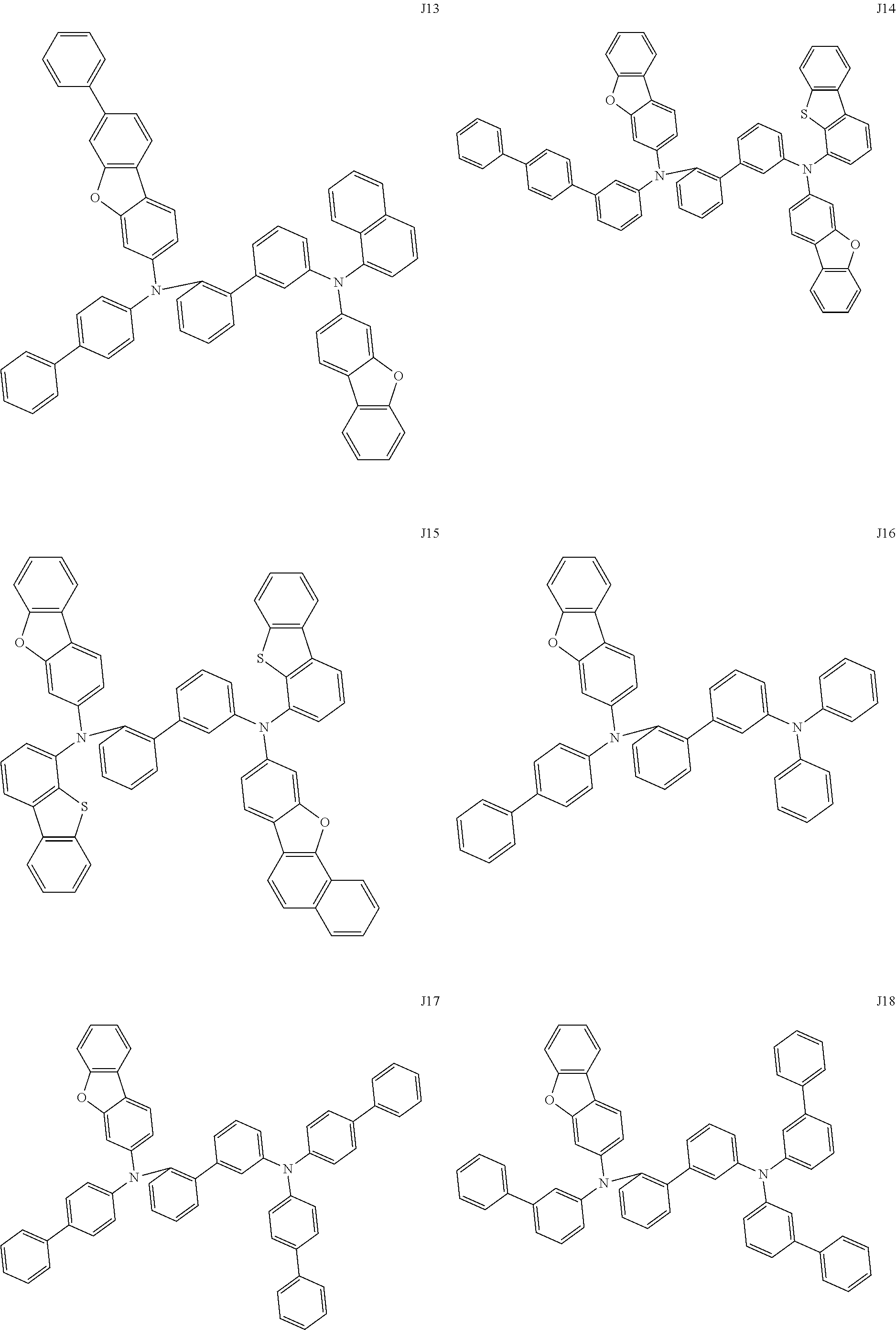

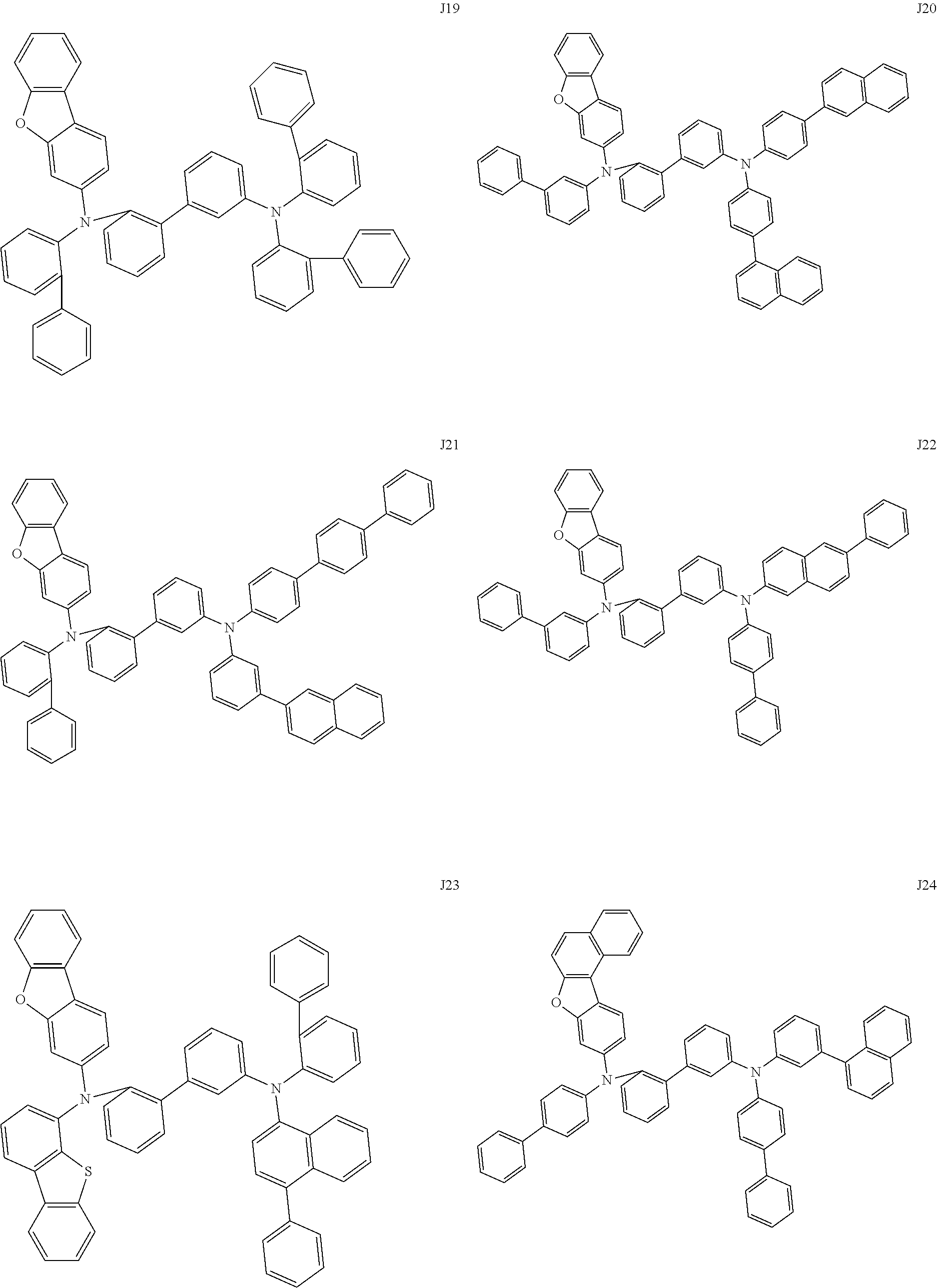

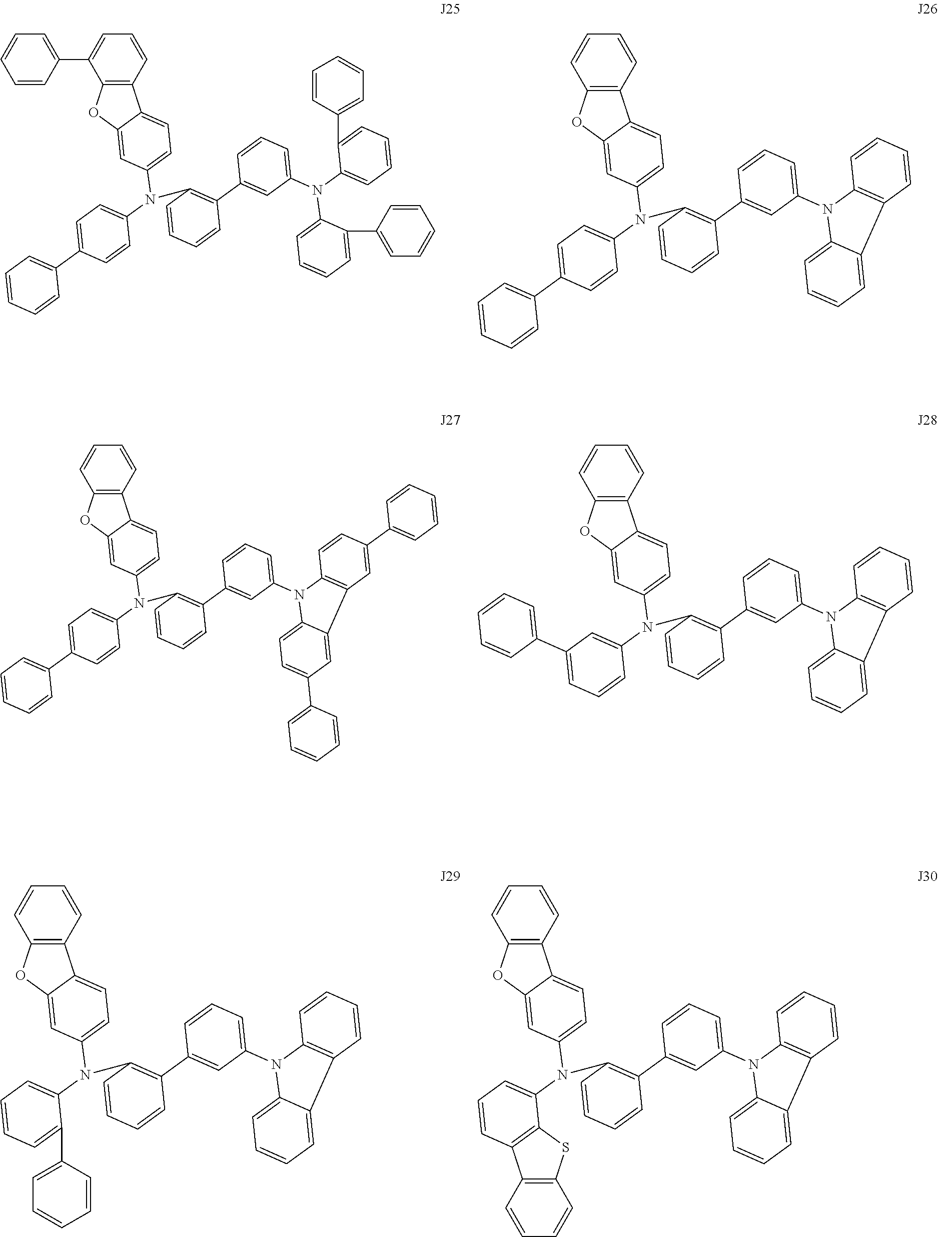

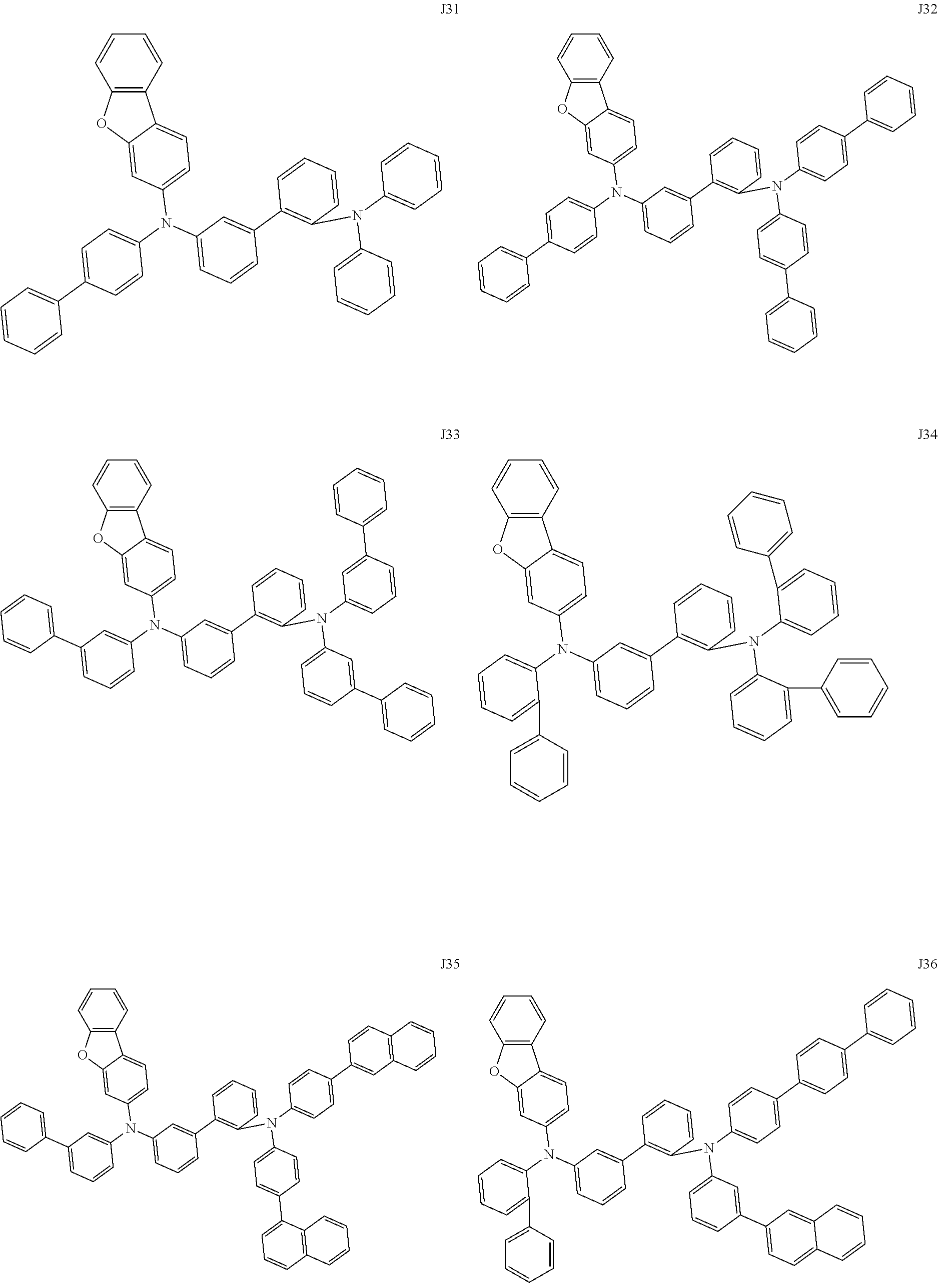

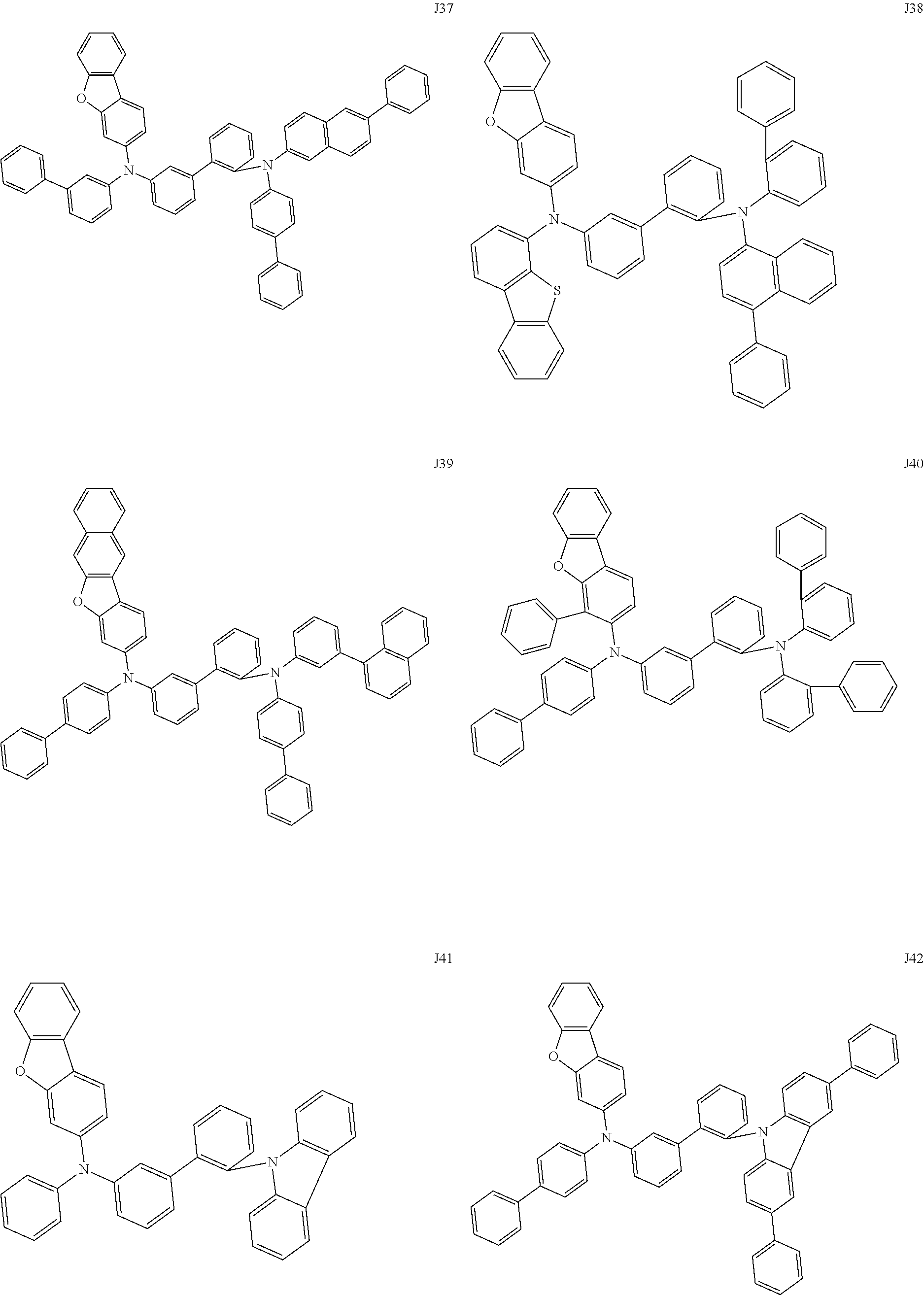

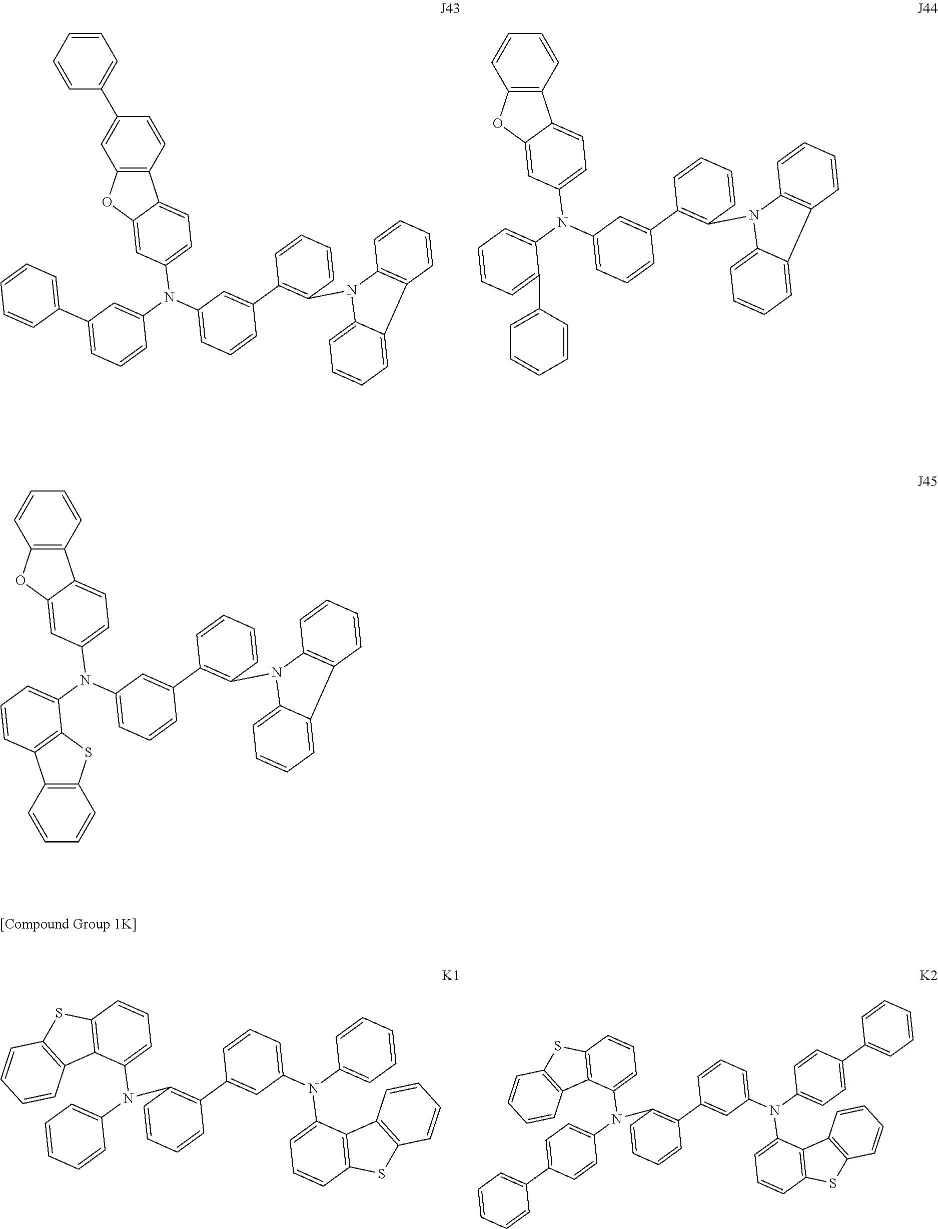 [SMILES: c1ccc(-c2cccc(N(c3cccc(-c4ccccc4-n4c5ccccc5c5ccccc54)c3)c3ccc4c(c3)oc3cc(-c5ccccc5)ccc34)c2)cc1 ; c1ccc(-c2ccccc2N(c2cccc(-c3ccccc3-n3c4ccccc4c4ccccc43)c2)c2ccc3c(c2)oc2ccccc23)cc1 ; c1cc(-c2ccccc2-n2c3ccccc3c3ccccc32)cc(N(c2ccc3c(c2)oc2ccccc23)c2cccc3c2sc2ccccc23)c1 ; c1ccc(N(c2cccc(-c3ccccc3N(c3ccccc3)c3cccc4sc5ccccc5c34)c2)c2cccc3sc4ccccc4c23)cc1 ; c1ccc(-c2ccc(N(c3cccc(-c4ccccc4N(c4ccc(-c5ccccc5)cc4)c4cccc5sc6ccccc6c45)c3)c3cccc4sc5ccccc5c34)cc2)cc1]

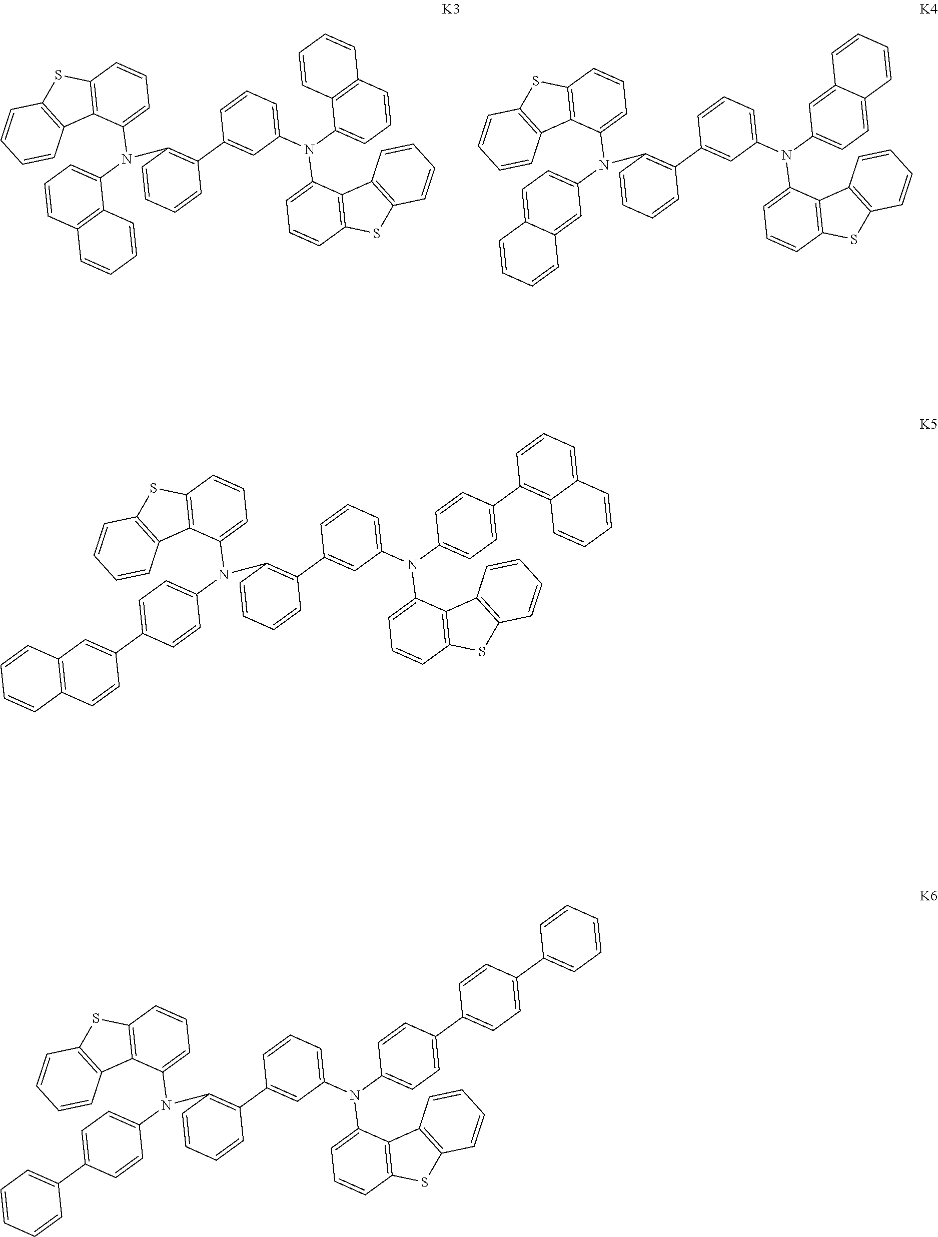 [SMILES: c1cc(-c2ccccc2N(c2cccc3ccccc23)c2cccc3sc4ccccc4c23)cc(N(c2cccc3ccccc23)c2cccc3sc4ccccc4c23)c1 ; c1cc(-c2ccccc2N(c2ccc3ccccc3c2)c2cccc3sc4ccccc4c23)cc(N(c2ccc3ccccc3c2)c2cccc3sc4ccccc4c23)c1 ; c1cc(-c2ccccc2N(c2ccc(-c3ccc4ccccc4c3)cc2)c2cccc3sc4ccccc4c23)cc(N(c2ccc(-c3cccc4ccccc34)cc2)c2cccc3sc4ccccc4c23)c1 ; c1ccc(-c2ccc(-c3ccc(N(c4cccc(-c5ccccc5N(c5ccc(-c6ccccc6)cc5)c5cccc6sc7ccccc7c56)c4)c4cccc5sc6ccccc6c45)cc3)cc2)cc1]

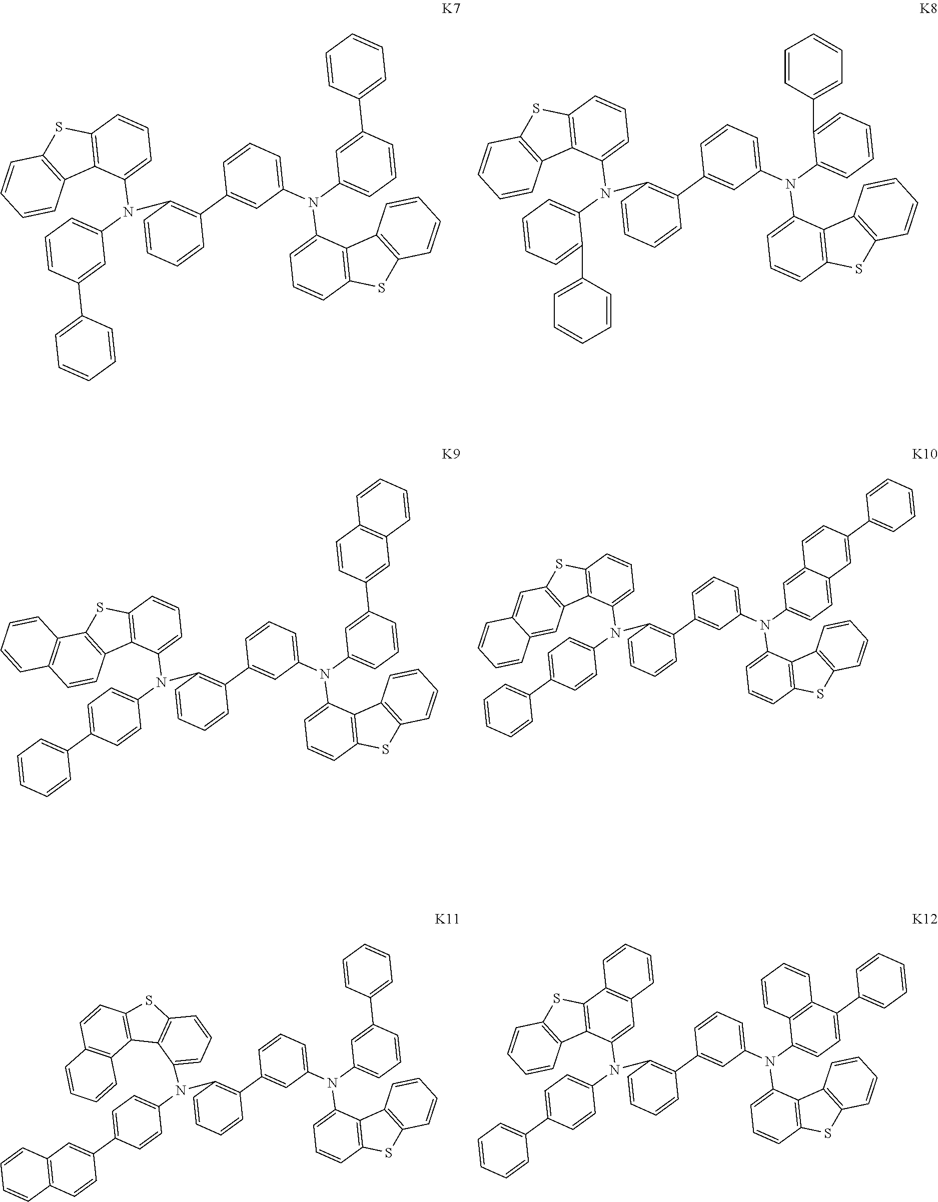

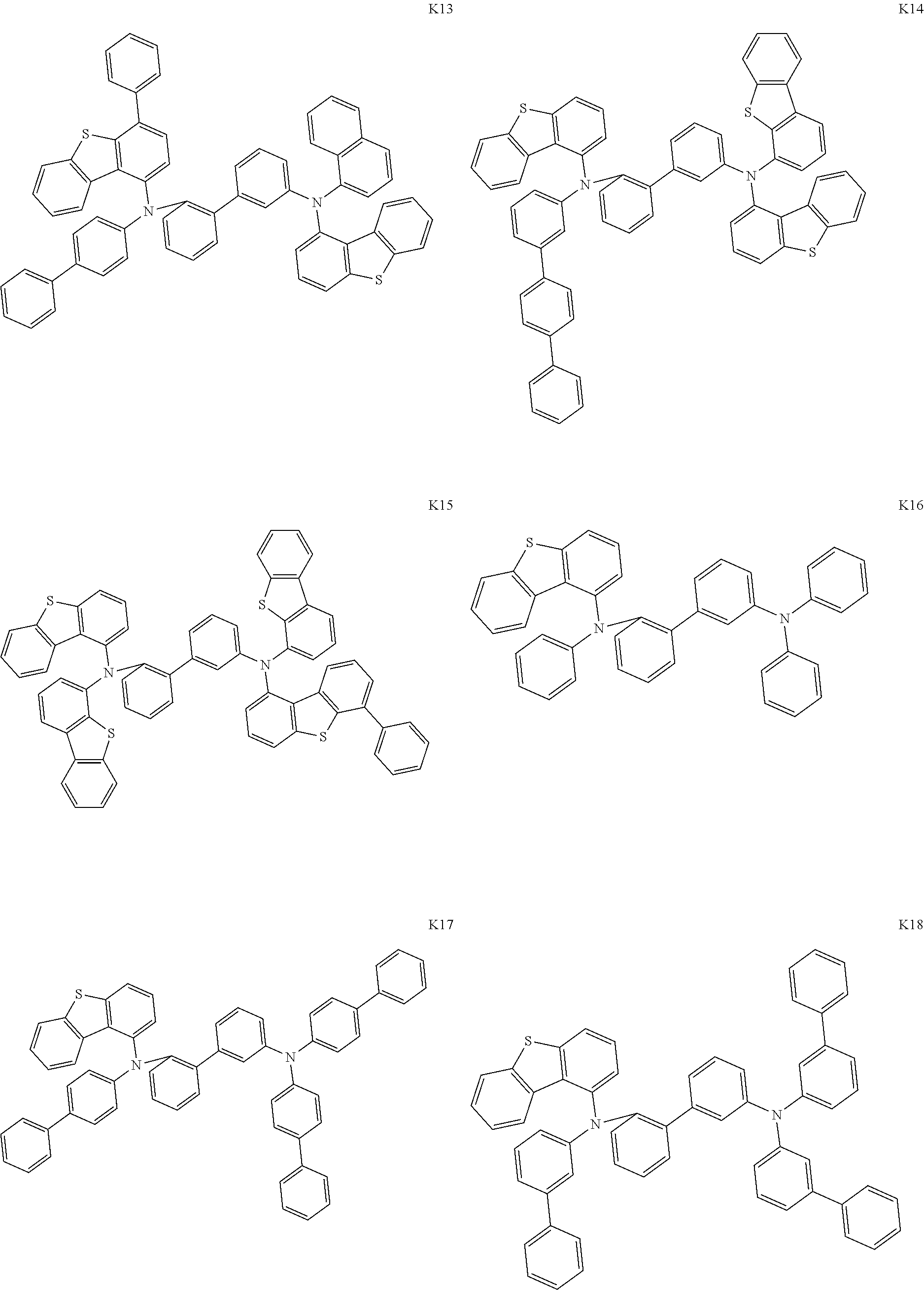

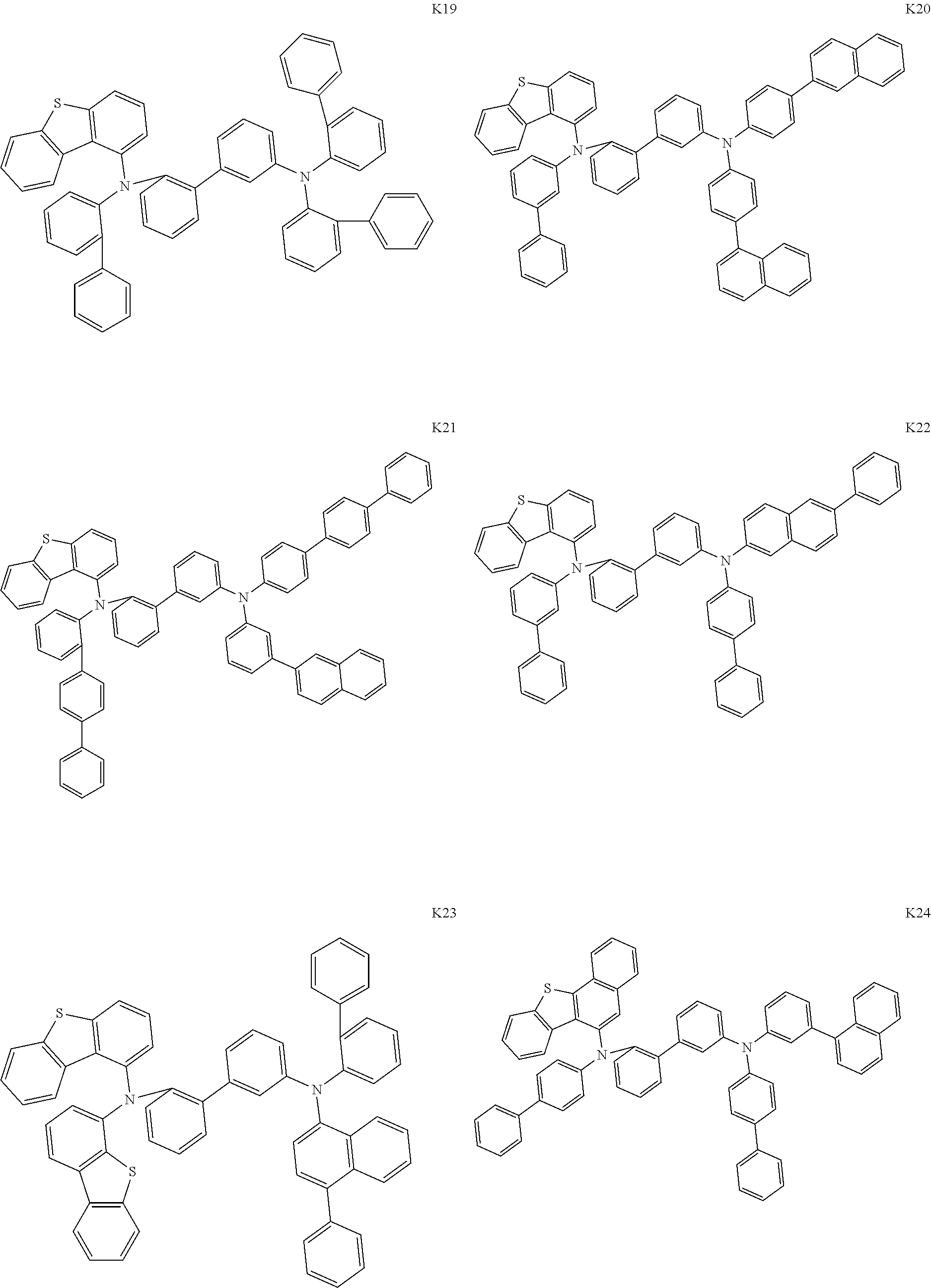

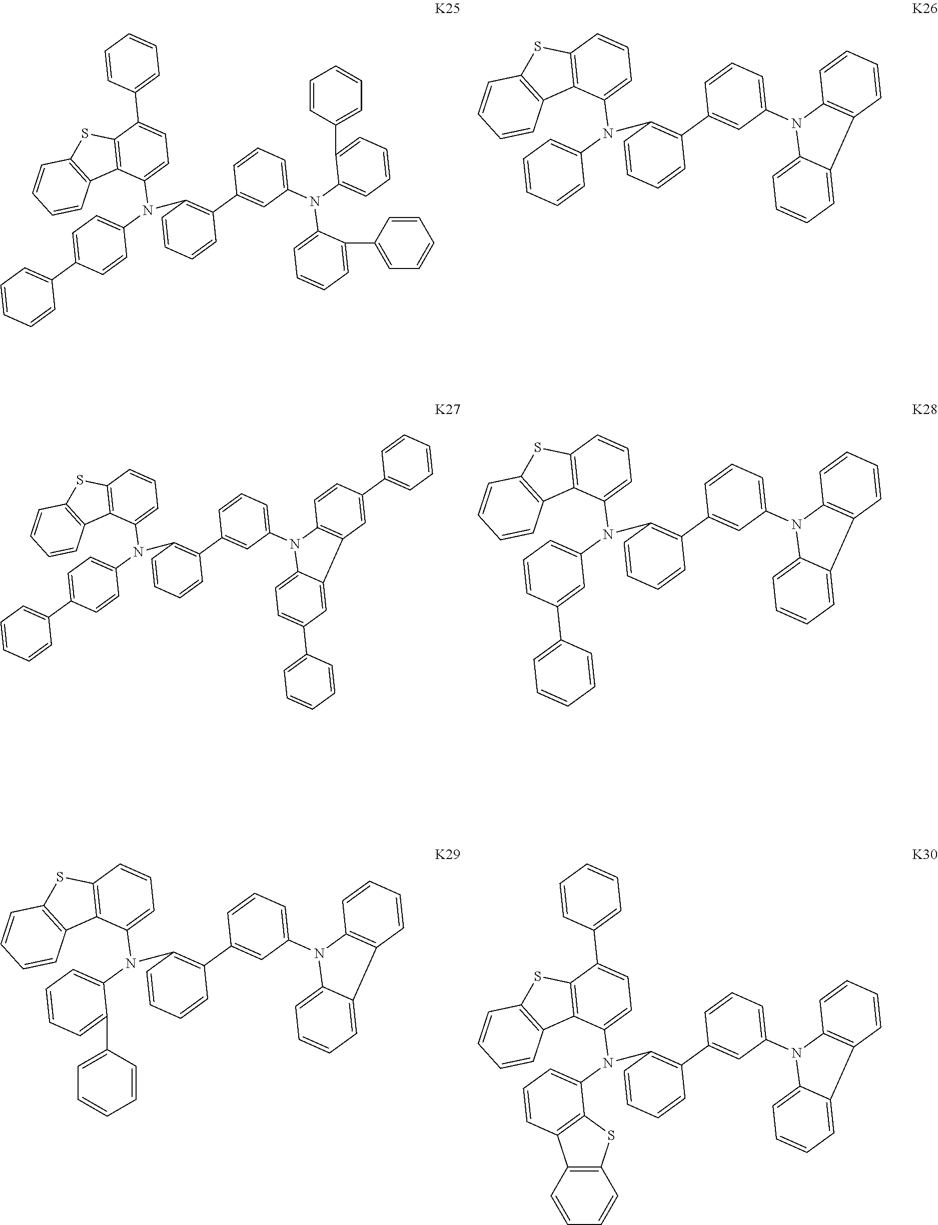

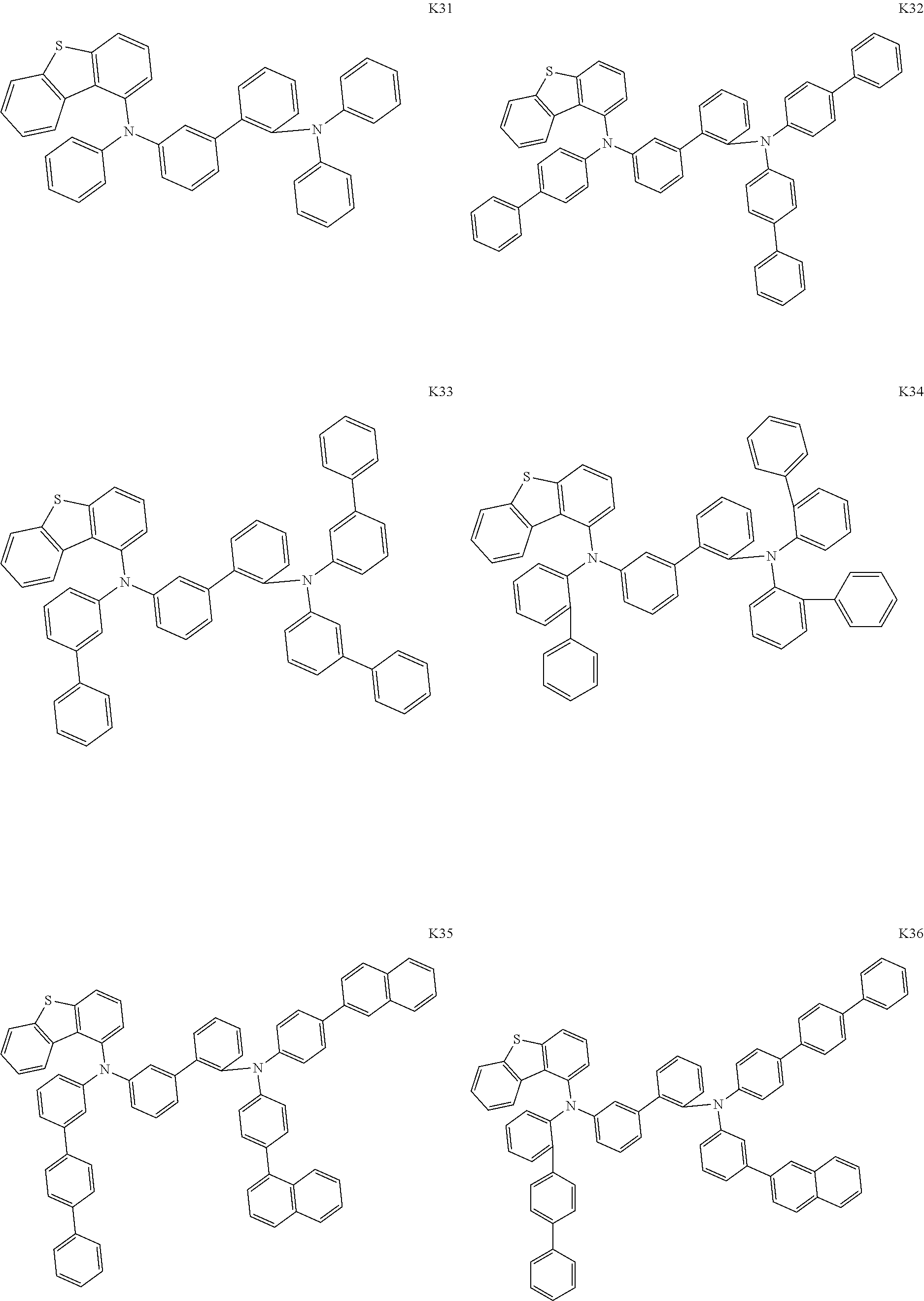

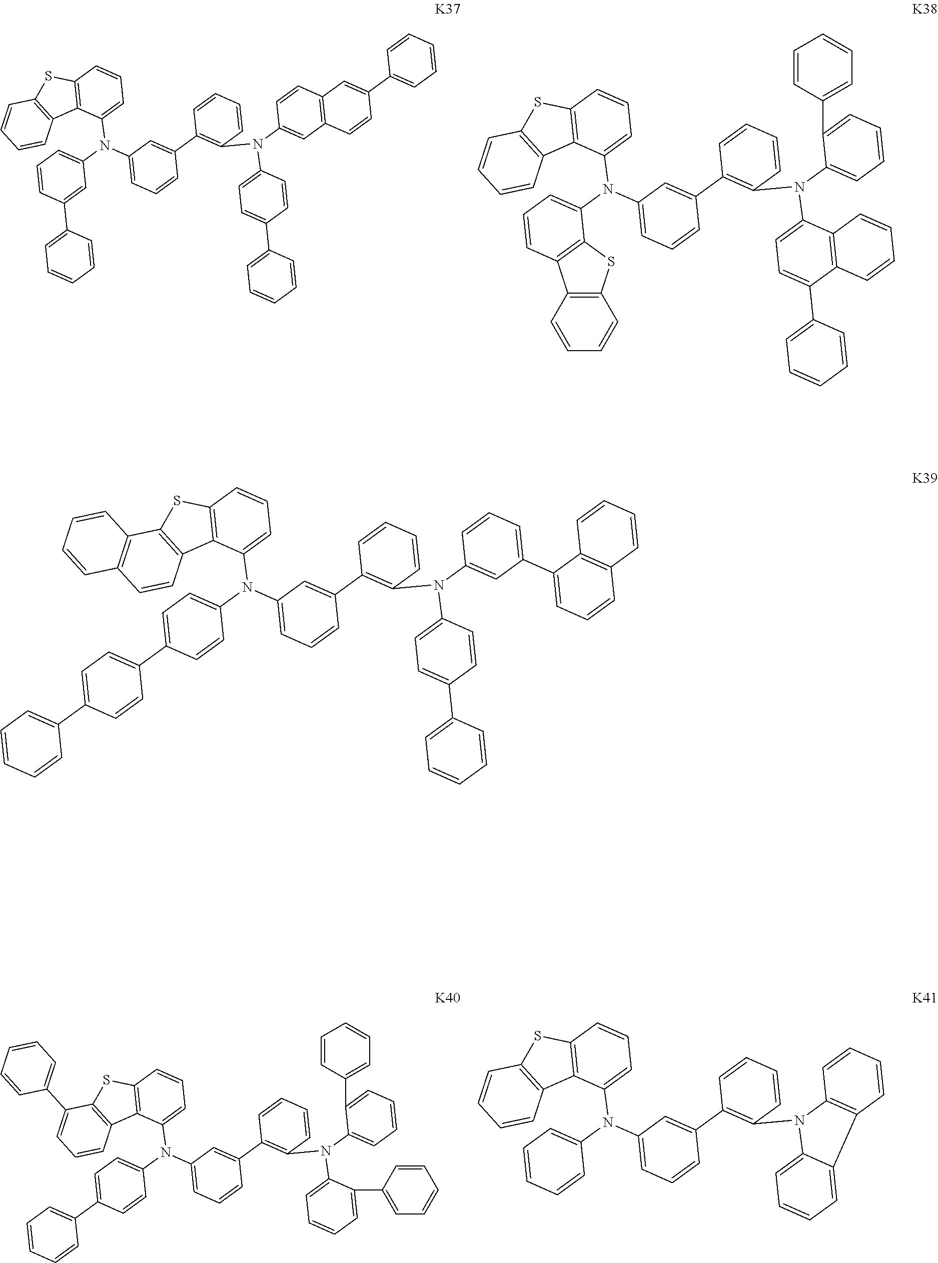

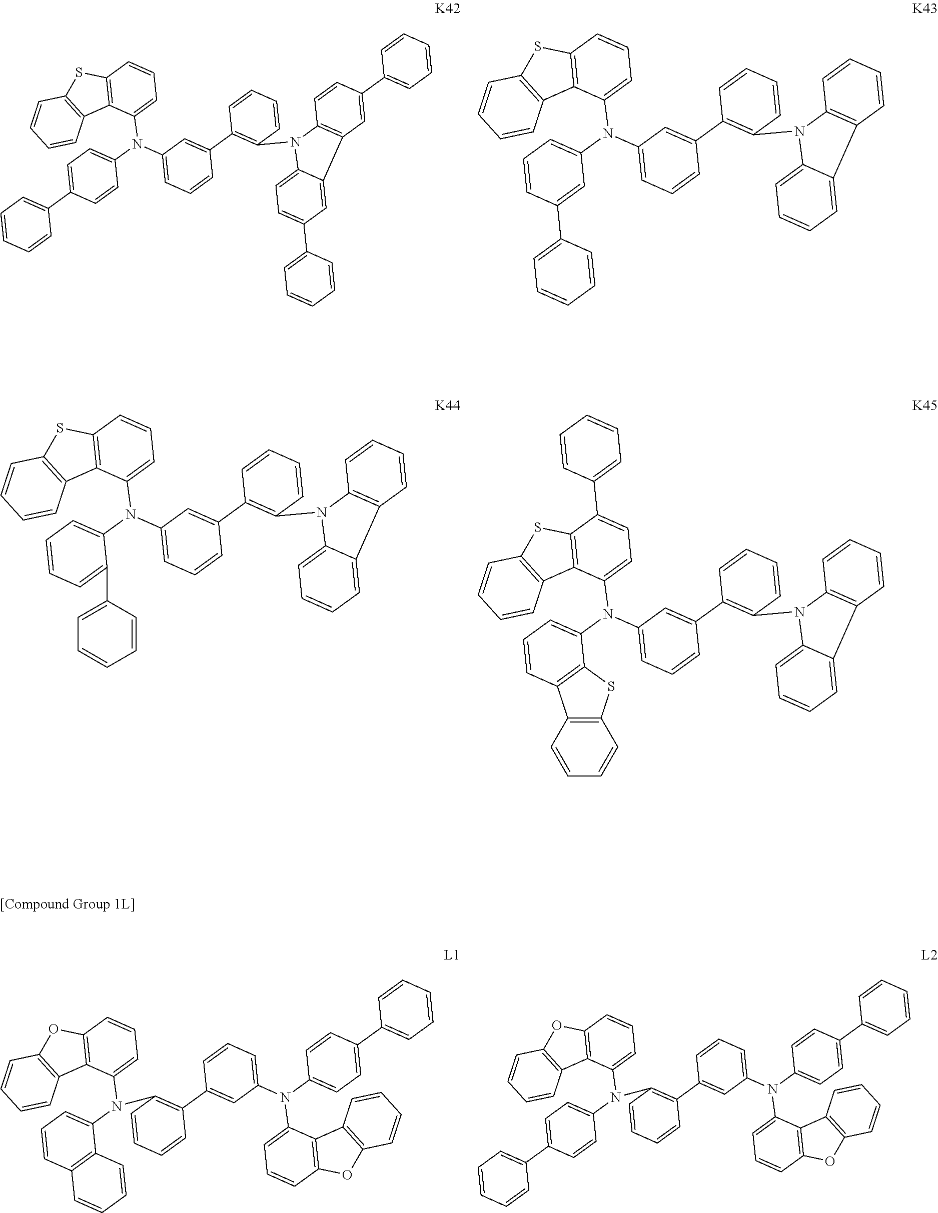

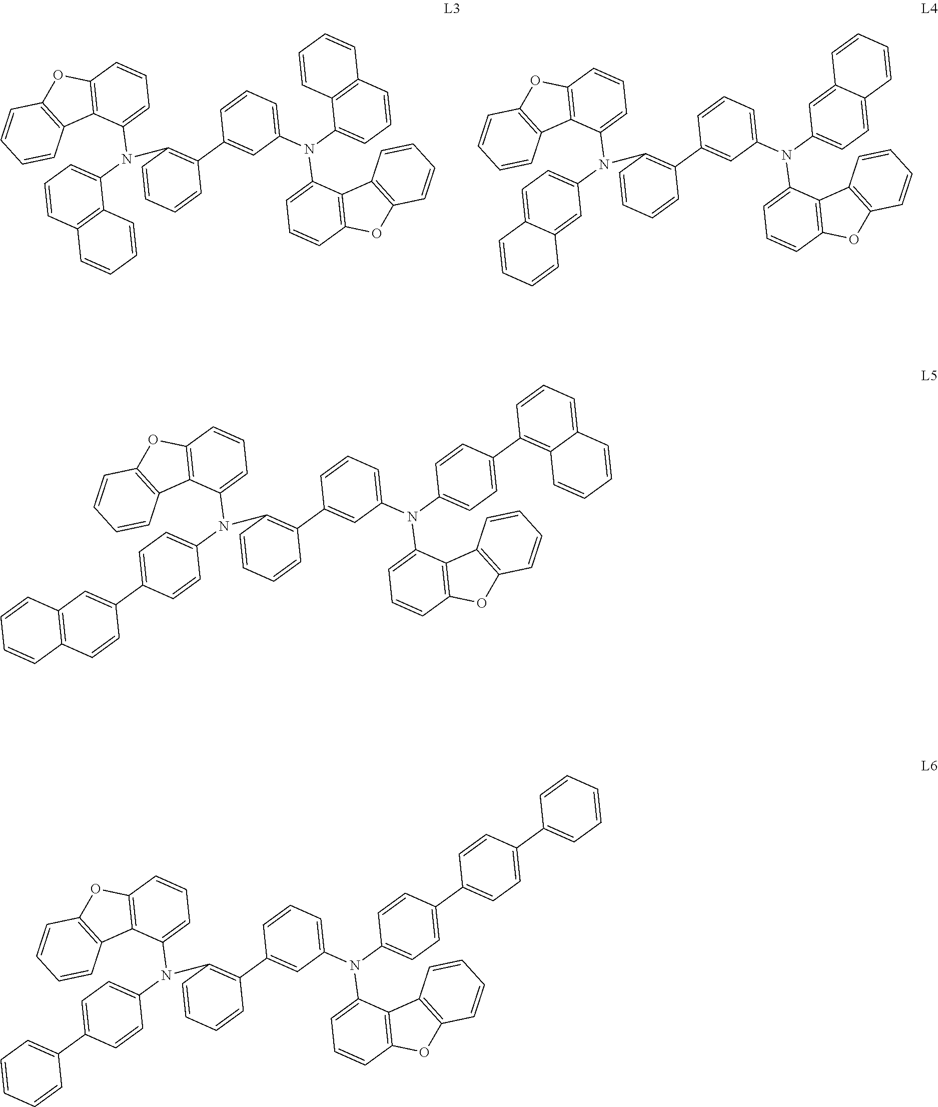 [SMILES: c1cc(-c2ccccc2N(c2cccc3ccccc23)c2cccc3oc4ccccc4c23)cc(N(c2cccc3ccccc23)c2cccc3oc4ccccc4c23)c1 ; c1cc(-c2ccccc2N(c2ccc3ccccc3c2)c2cccc3oc4ccccc4c23)cc(N(c2ccc3ccccc3c2)c2cccc3oc4ccccc4c23)c1 ; c1cc(-c2ccccc2N(c2ccc(-c3ccc4ccccc4c3)cc2)c2cccc3oc4ccccc4c23)cc(N(c2ccc(-c3cccc4ccccc34)cc2)c2cccc3oc4ccccc4c23)c1 ; c1ccc(-c2ccc(-c3ccc(N(c4cccc(-c5ccccc5N(c5ccc(-c6ccccc6)cc5)c5cccc6oc7ccccc7c56)c4)c4cccc5oc6ccccc6c45)cc3)cc2)cc1]

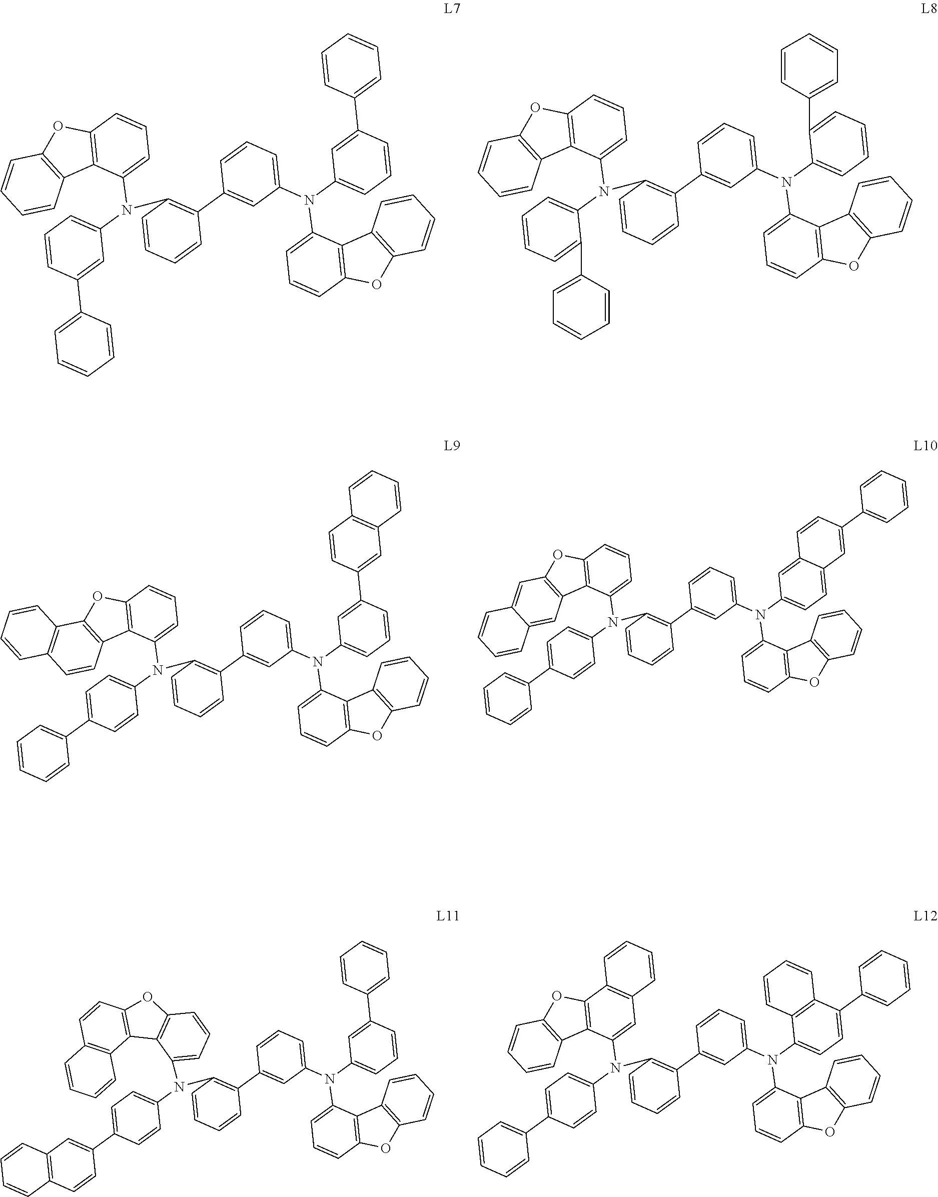

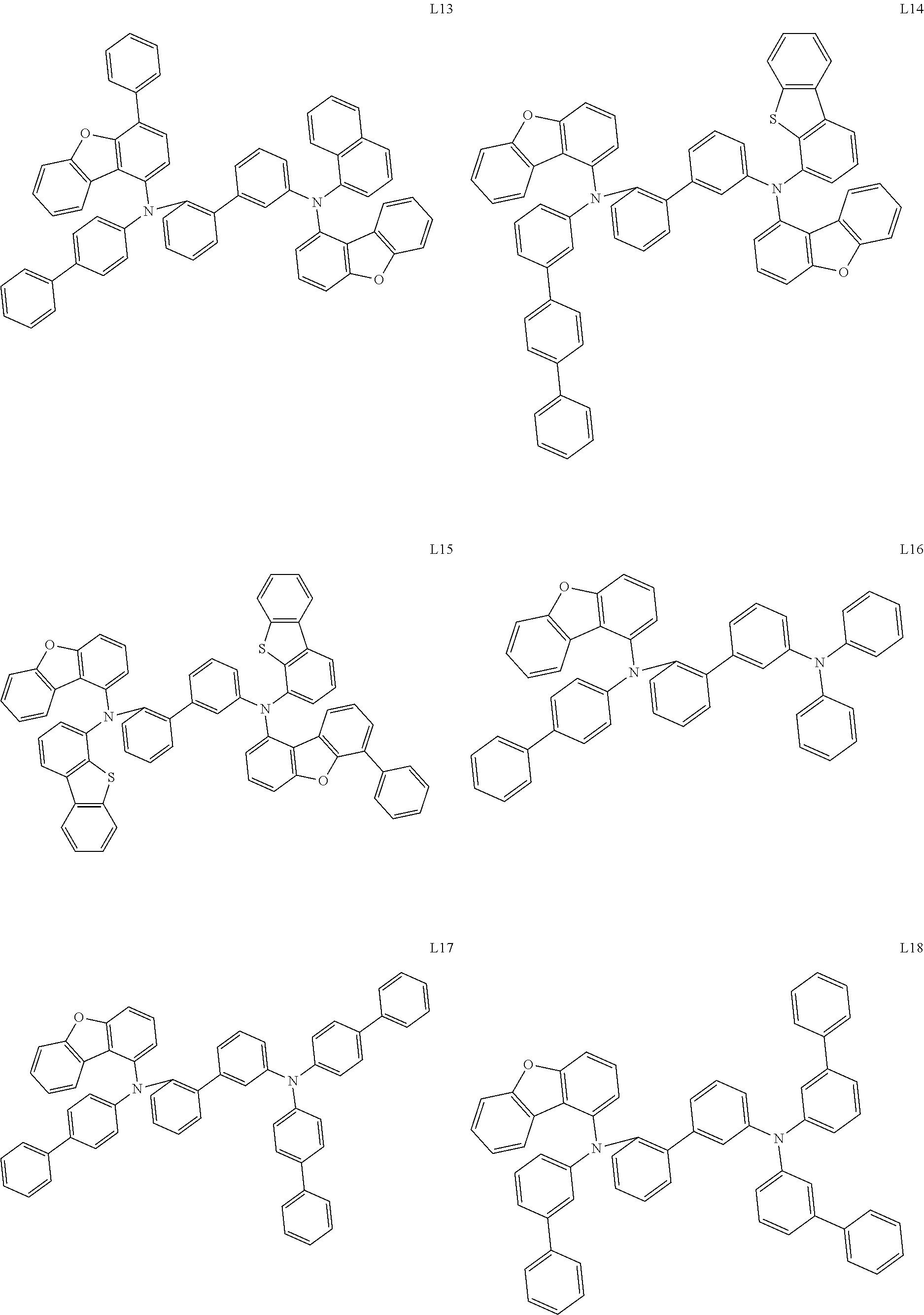

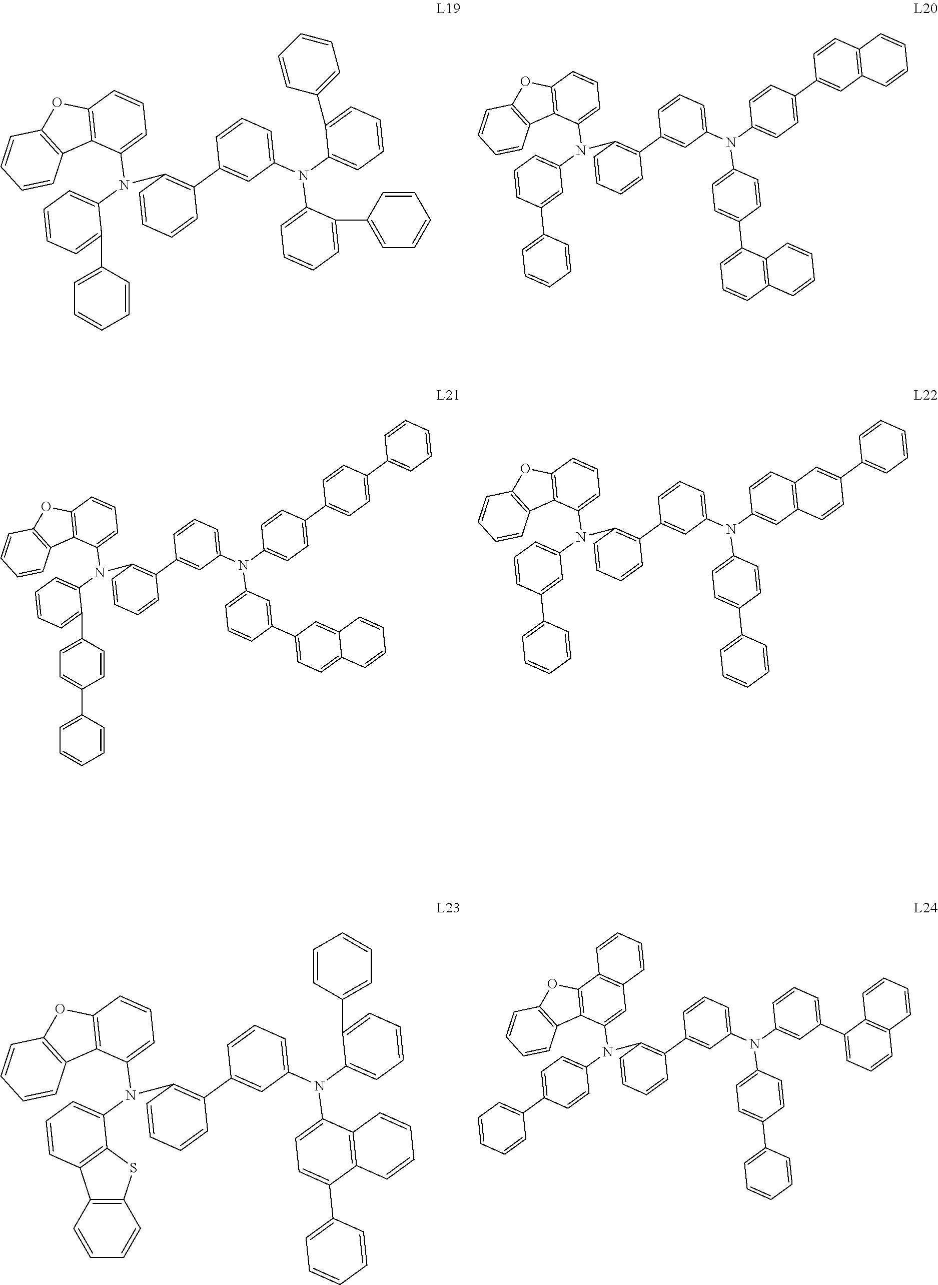

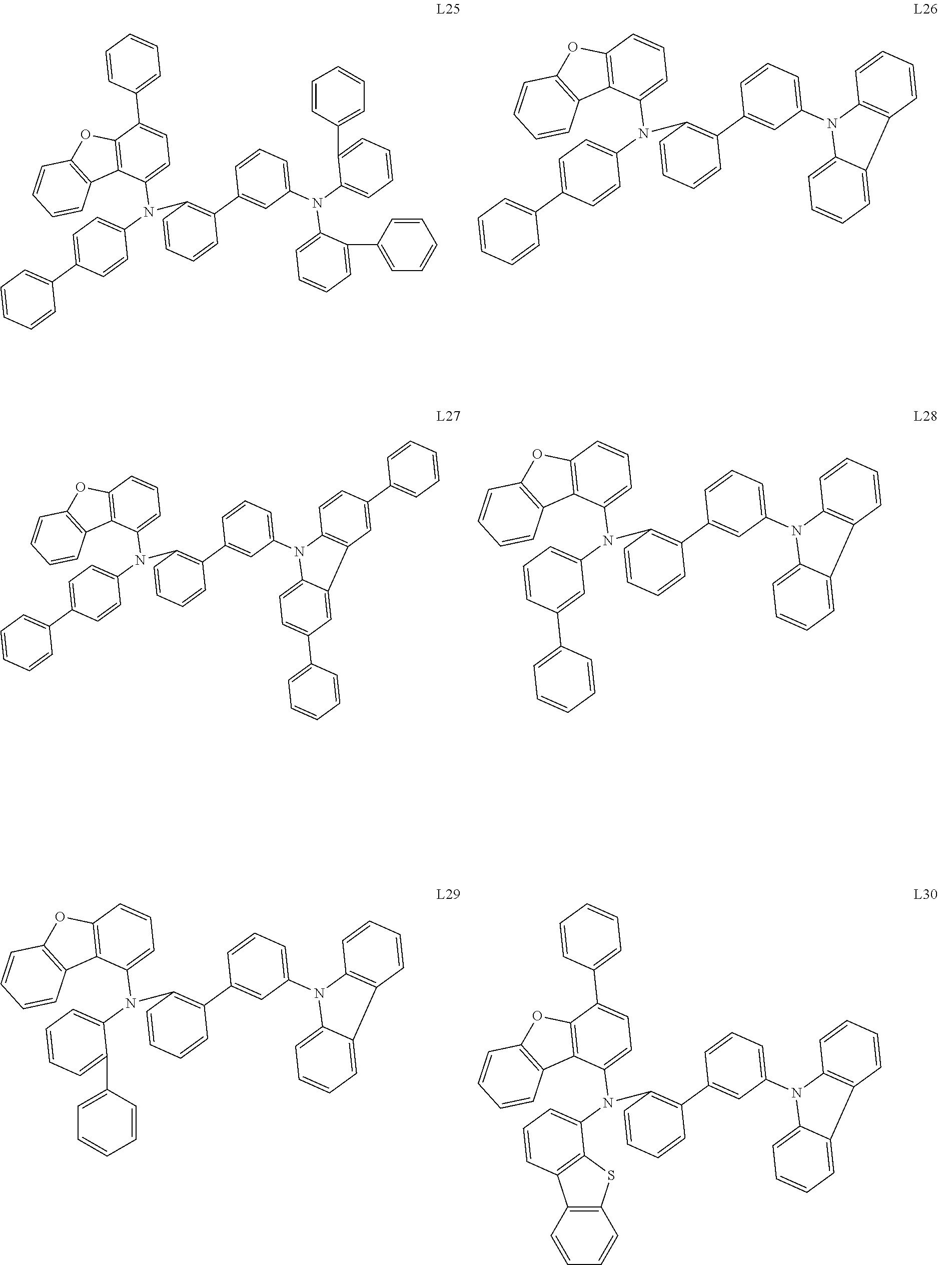

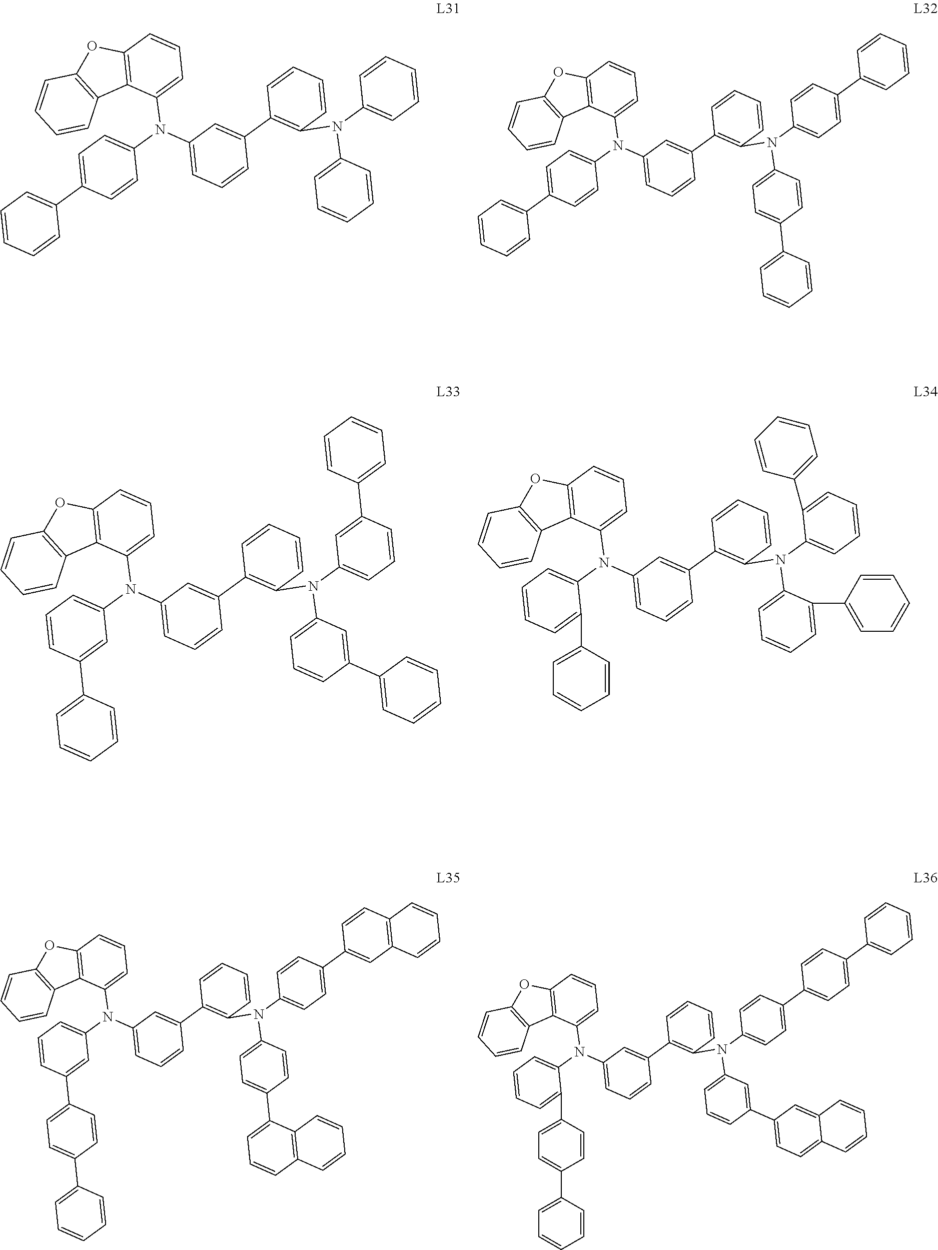

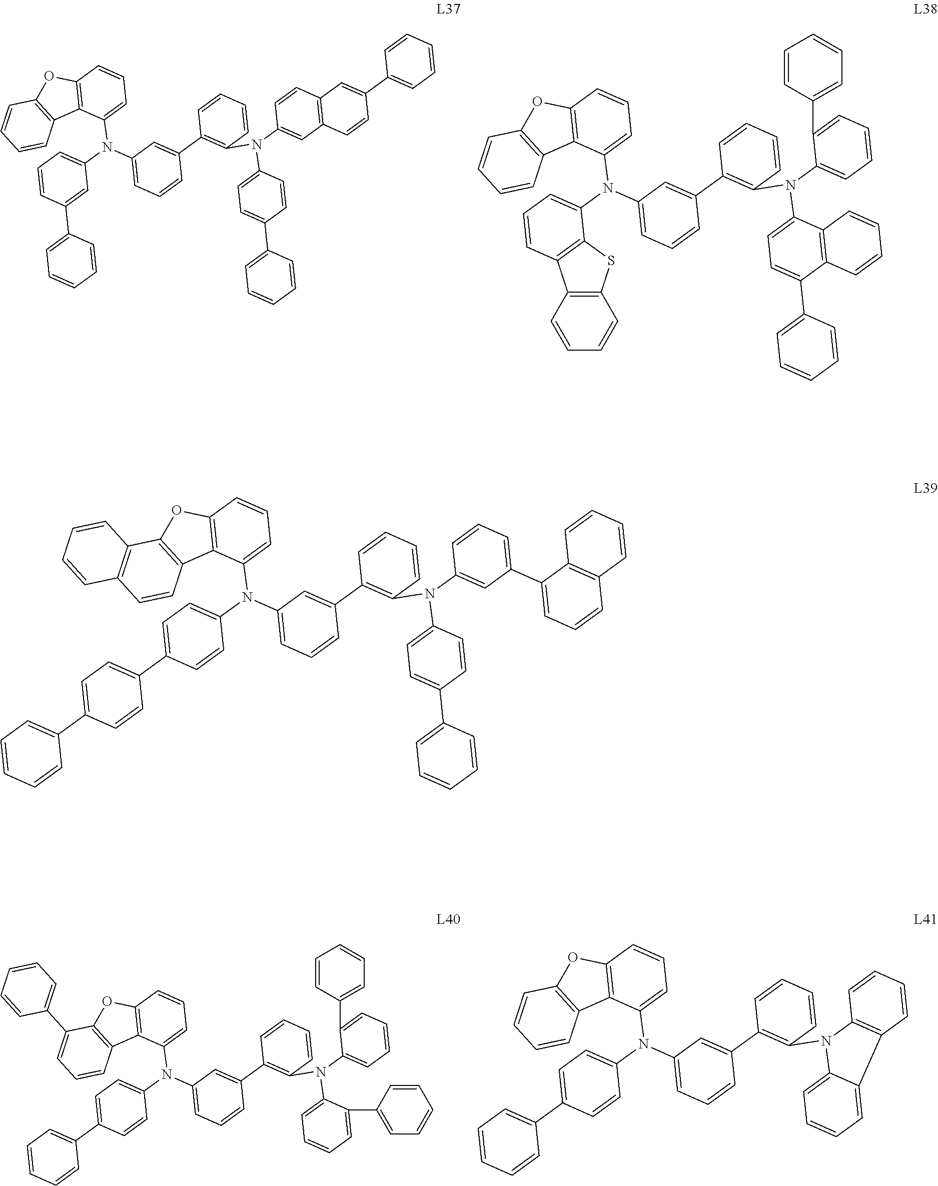

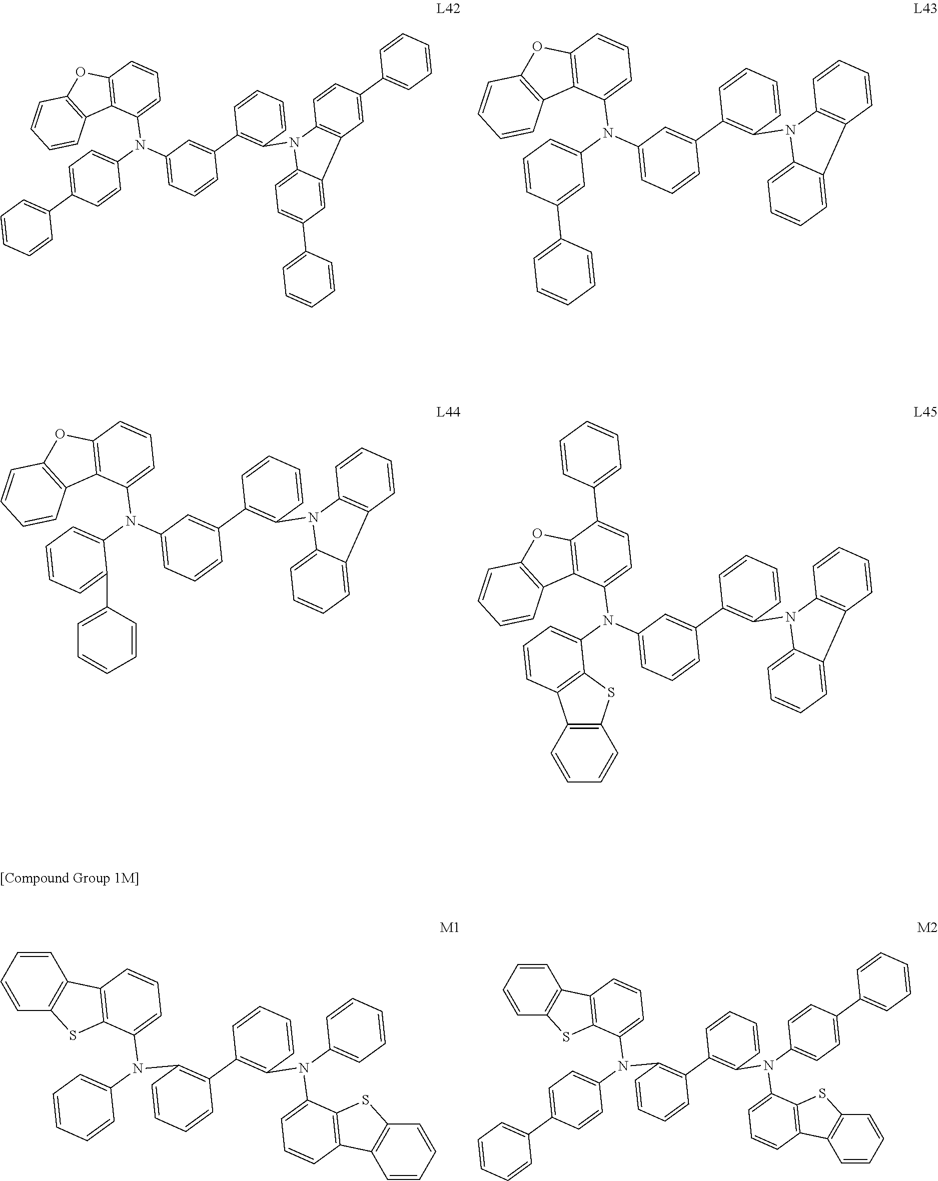

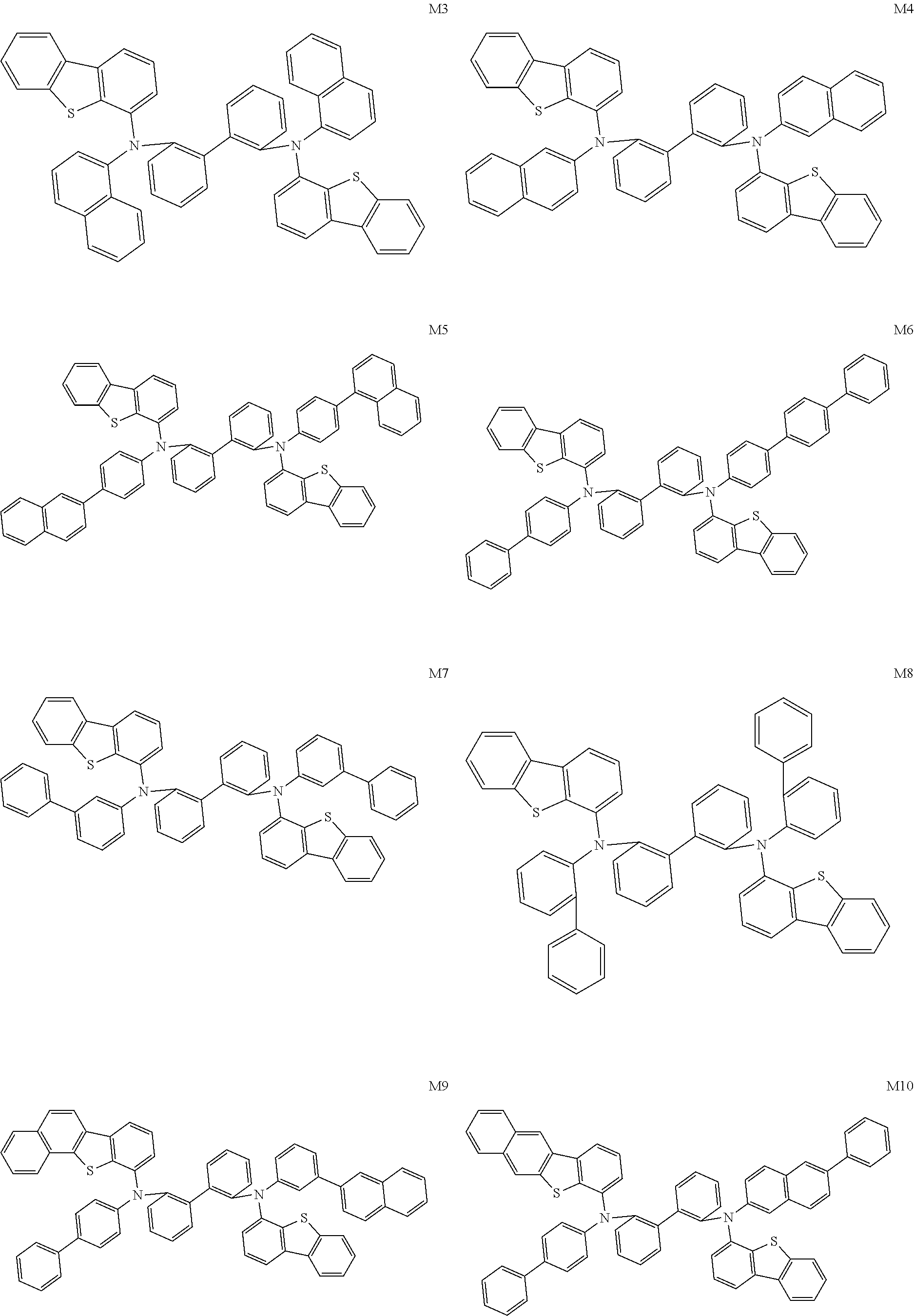

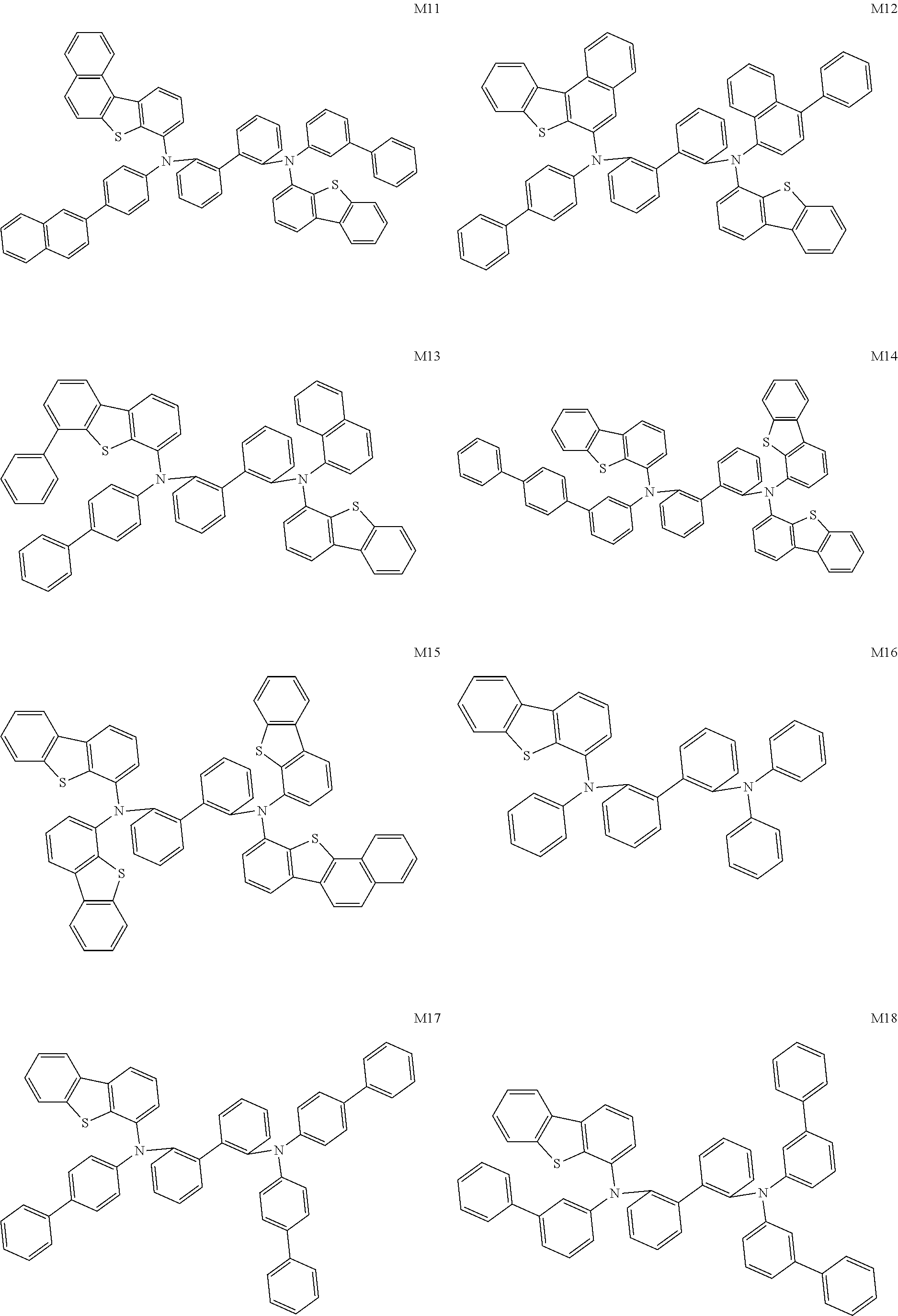

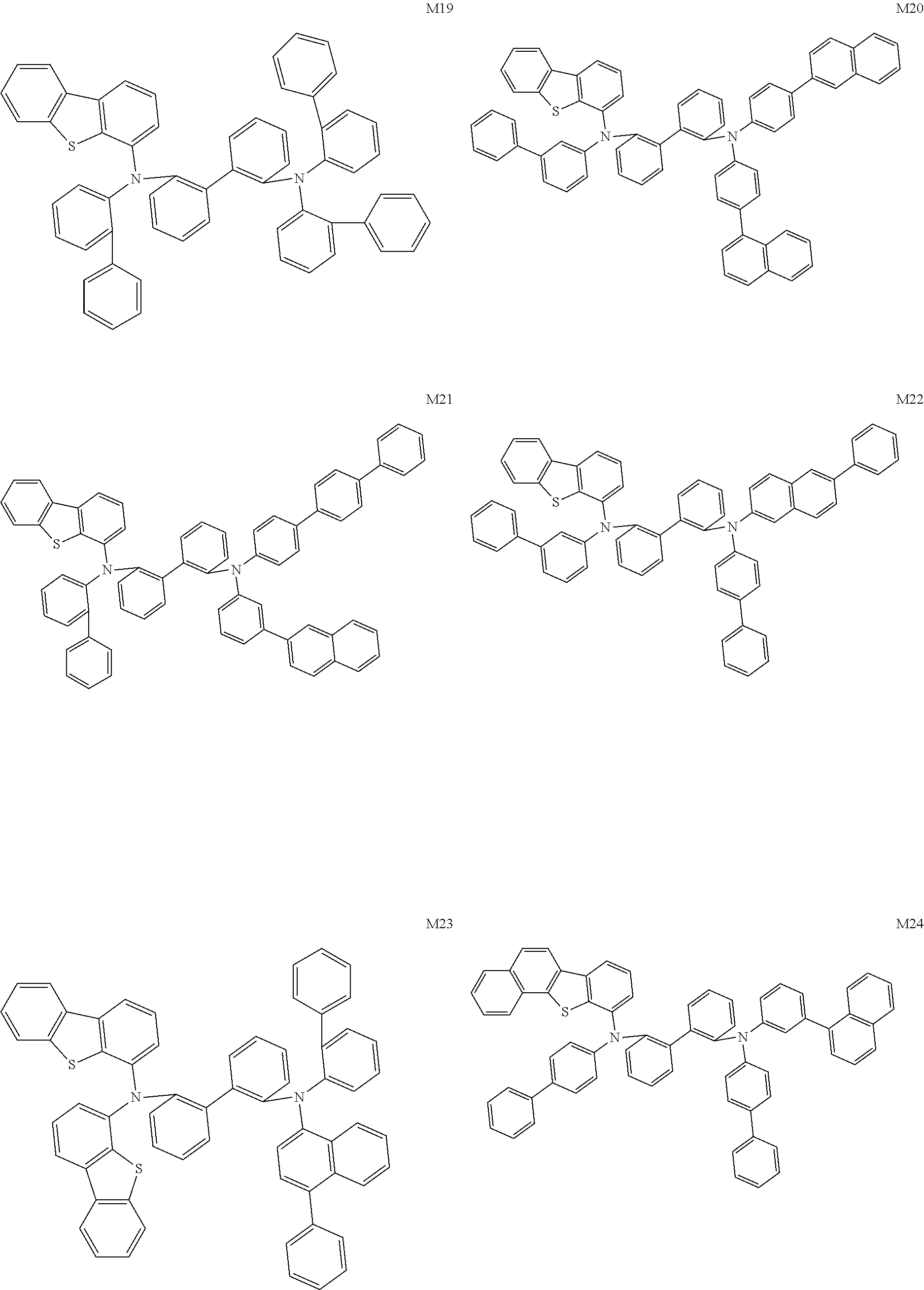

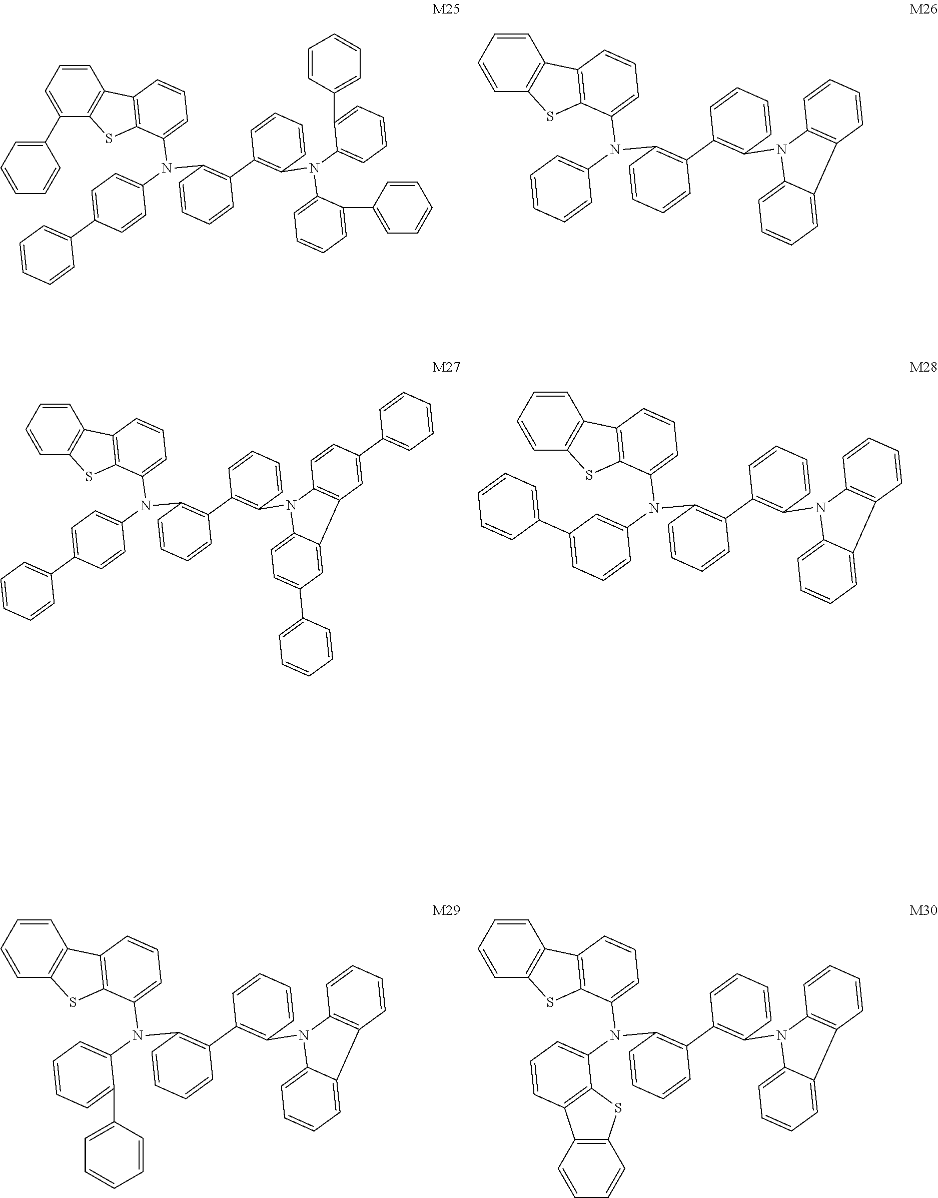

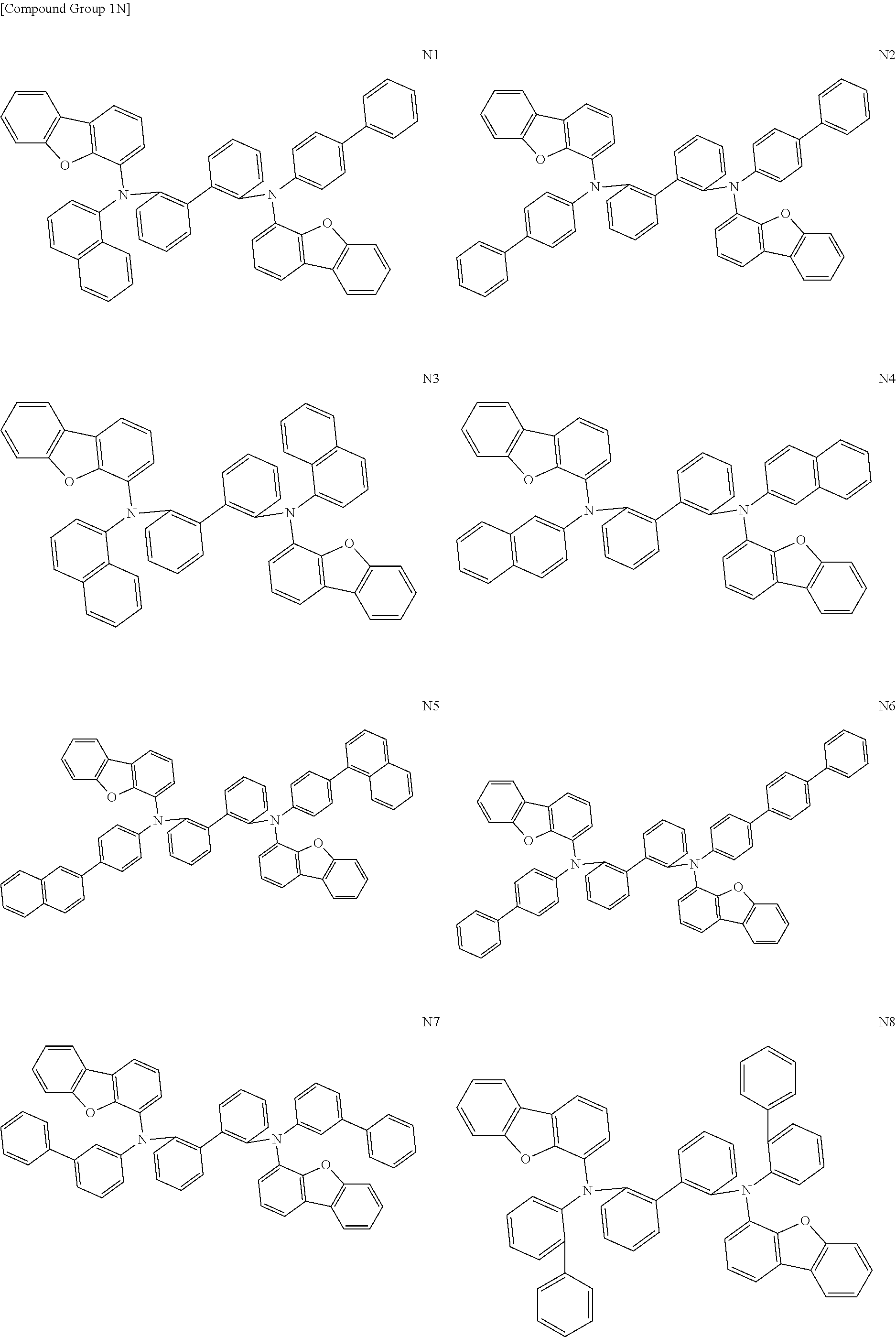

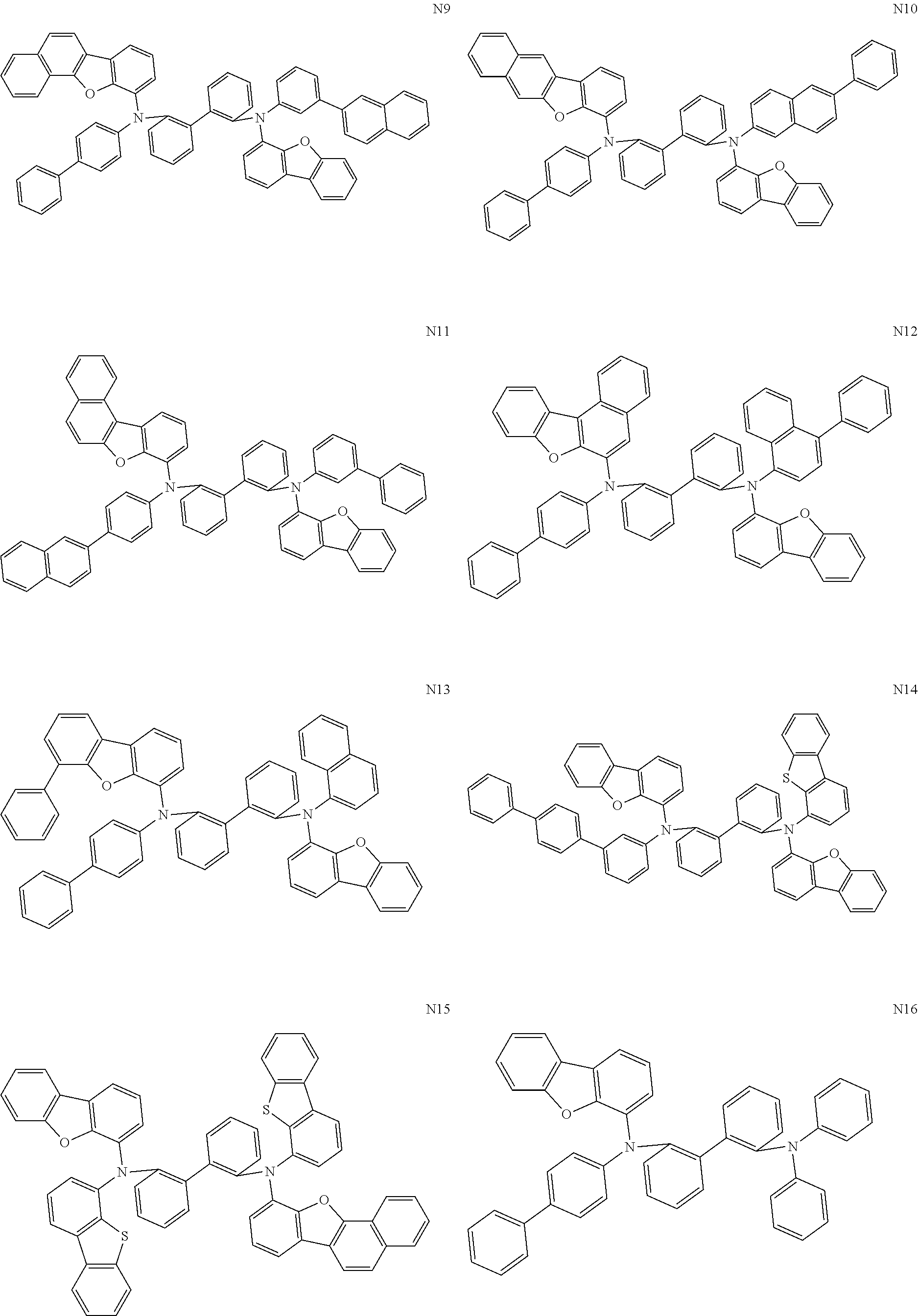

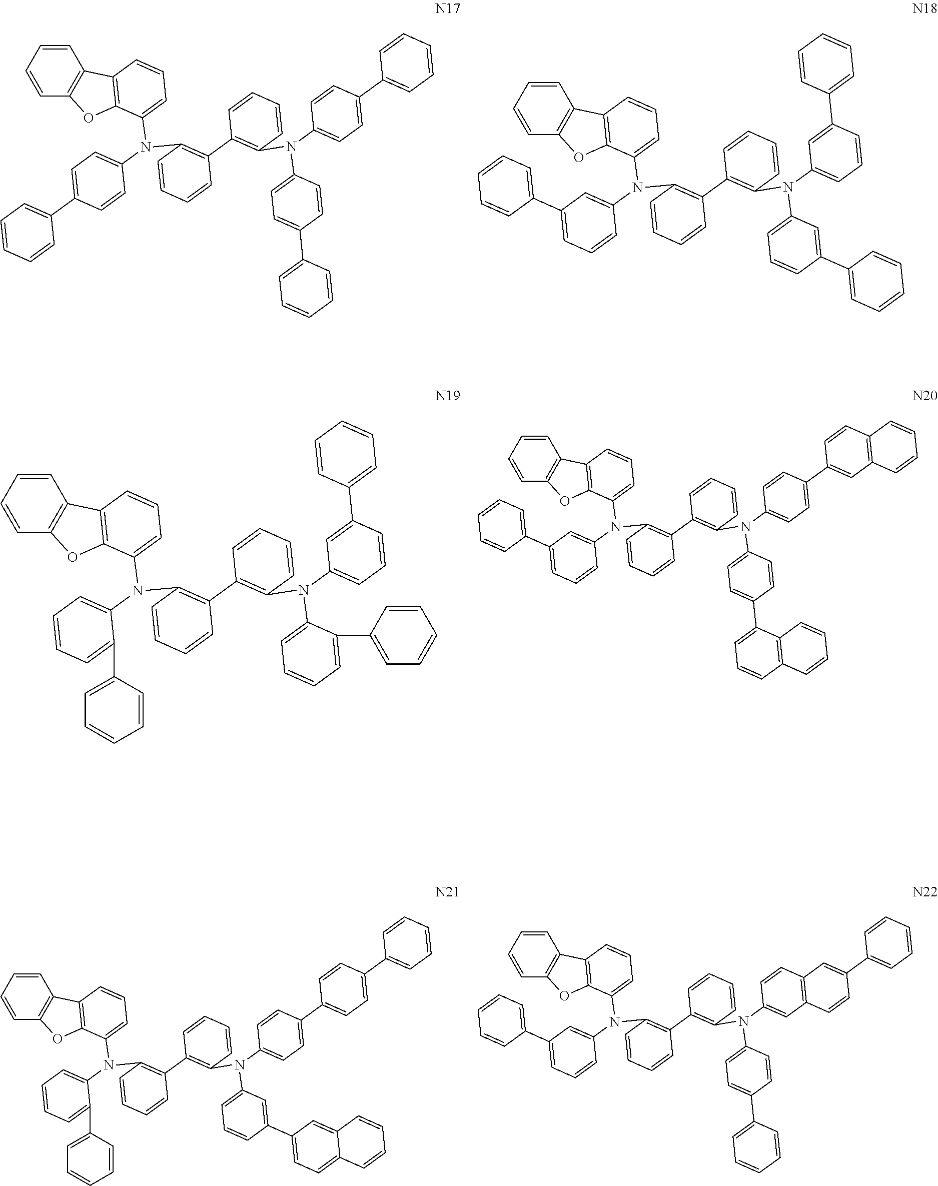

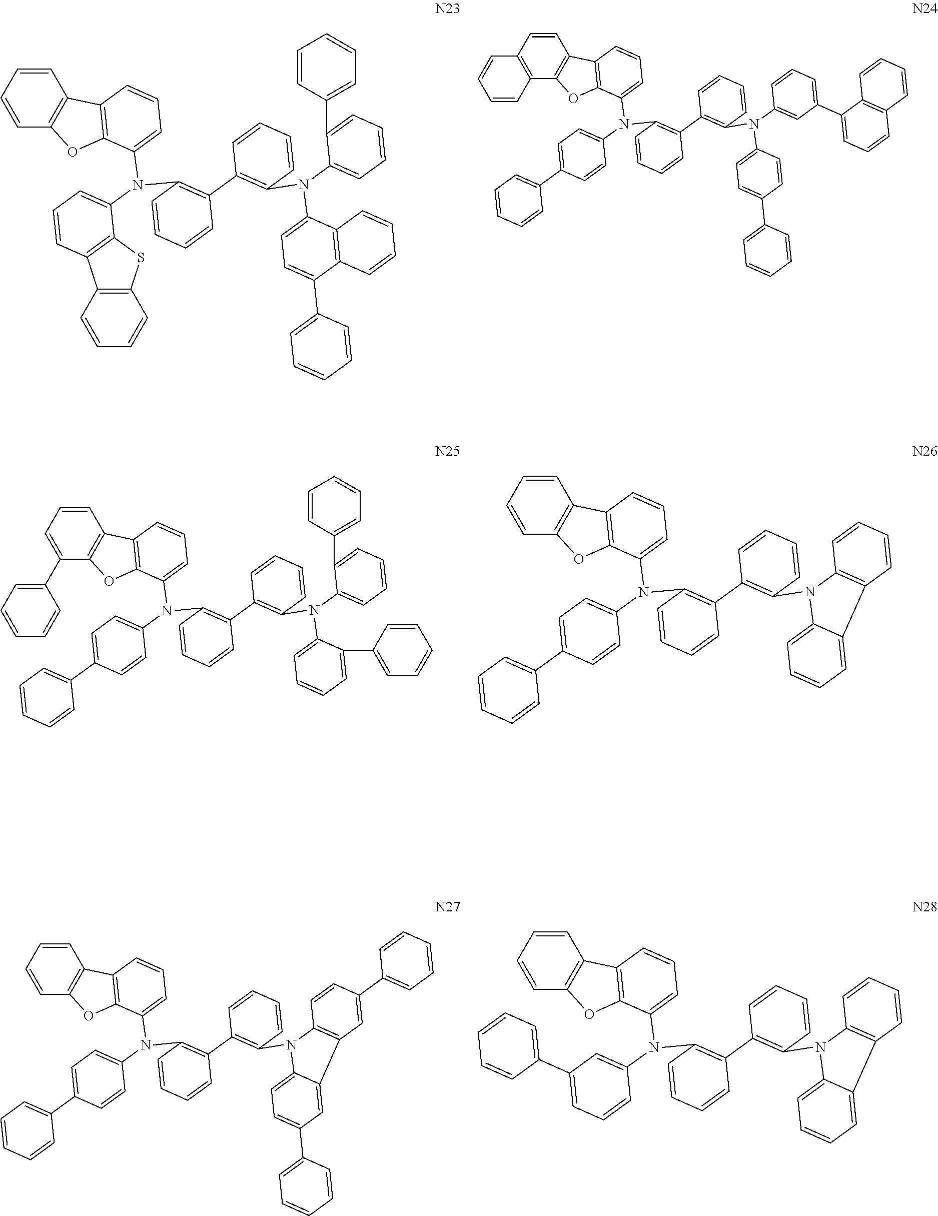

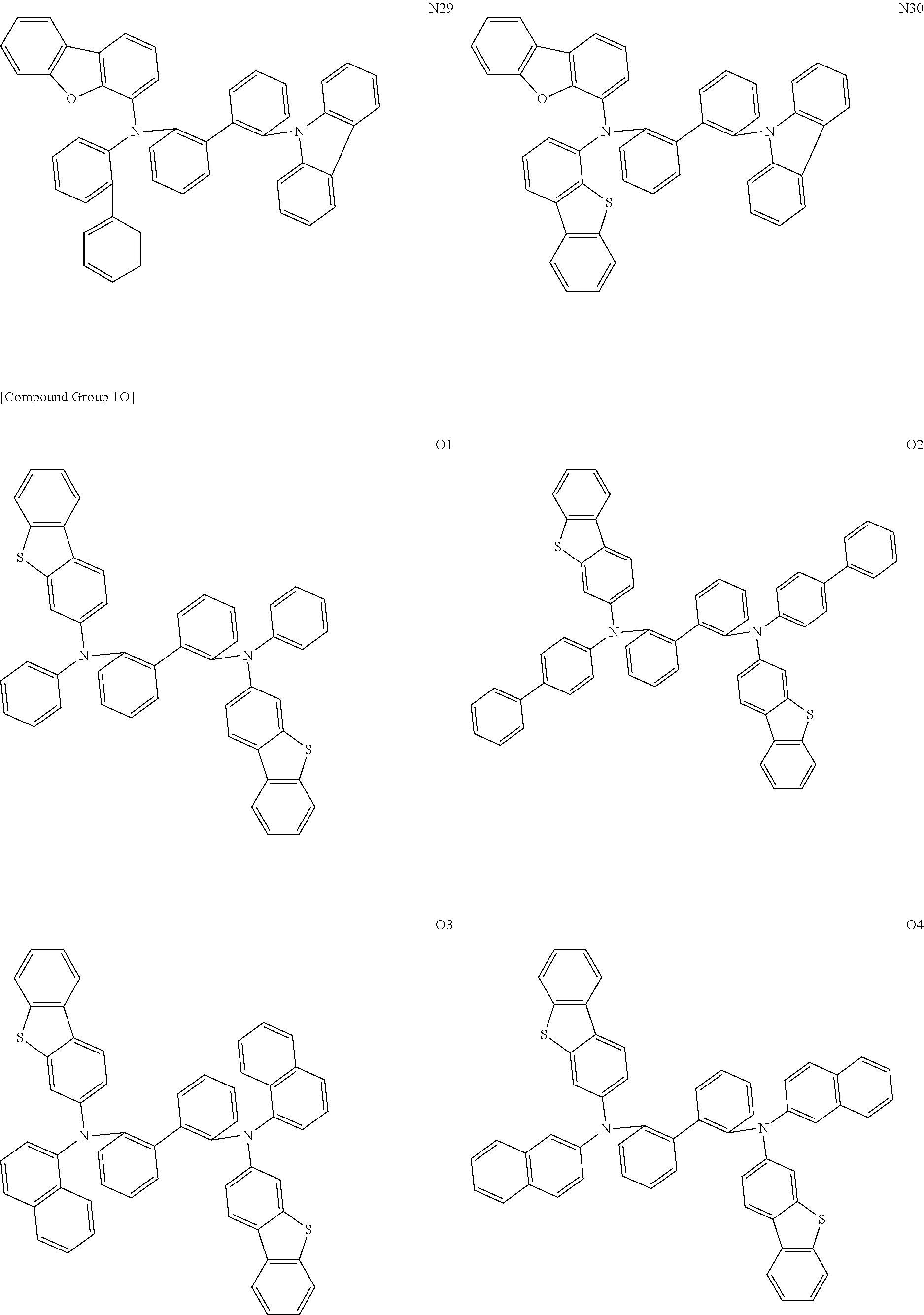

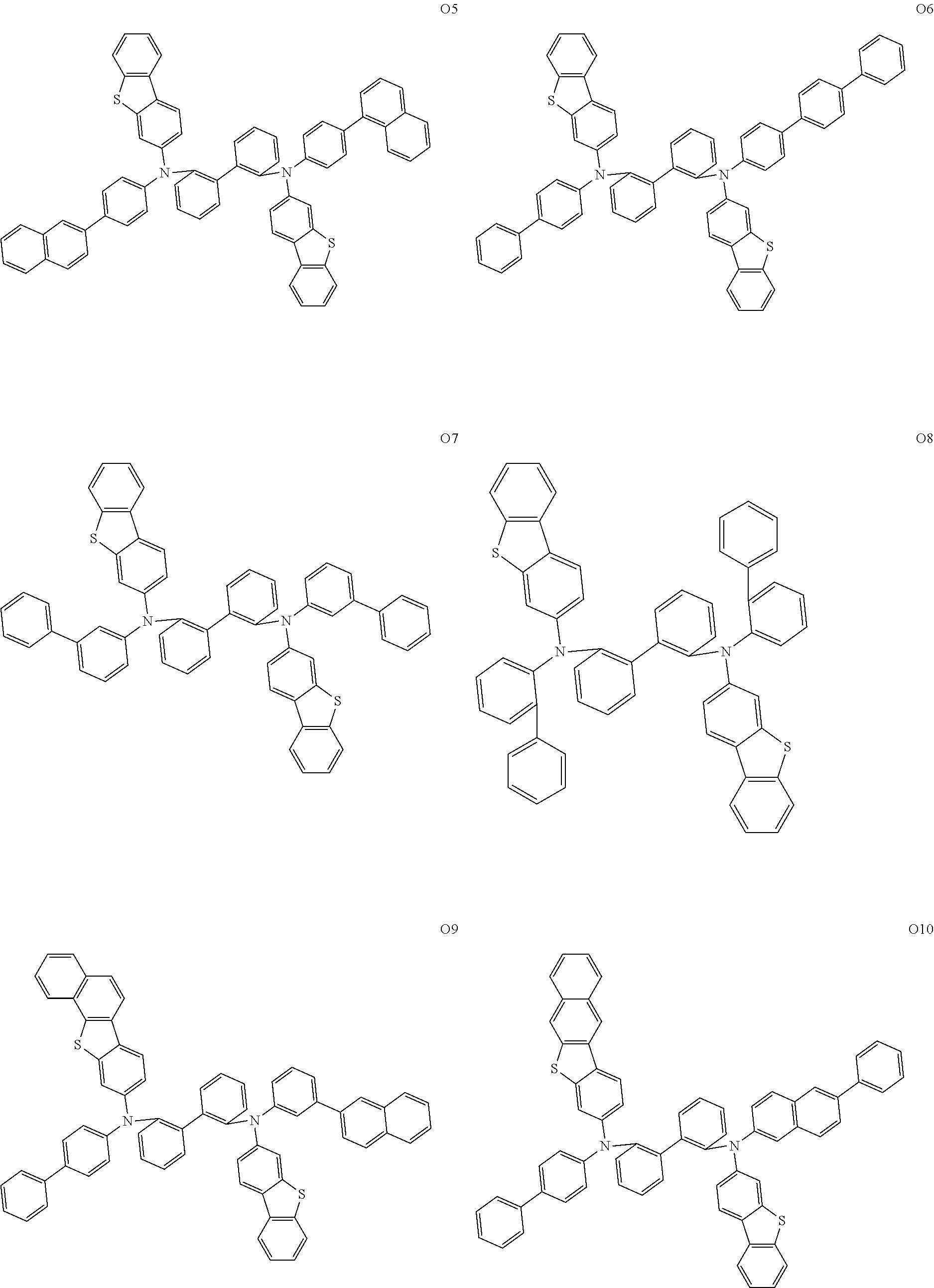

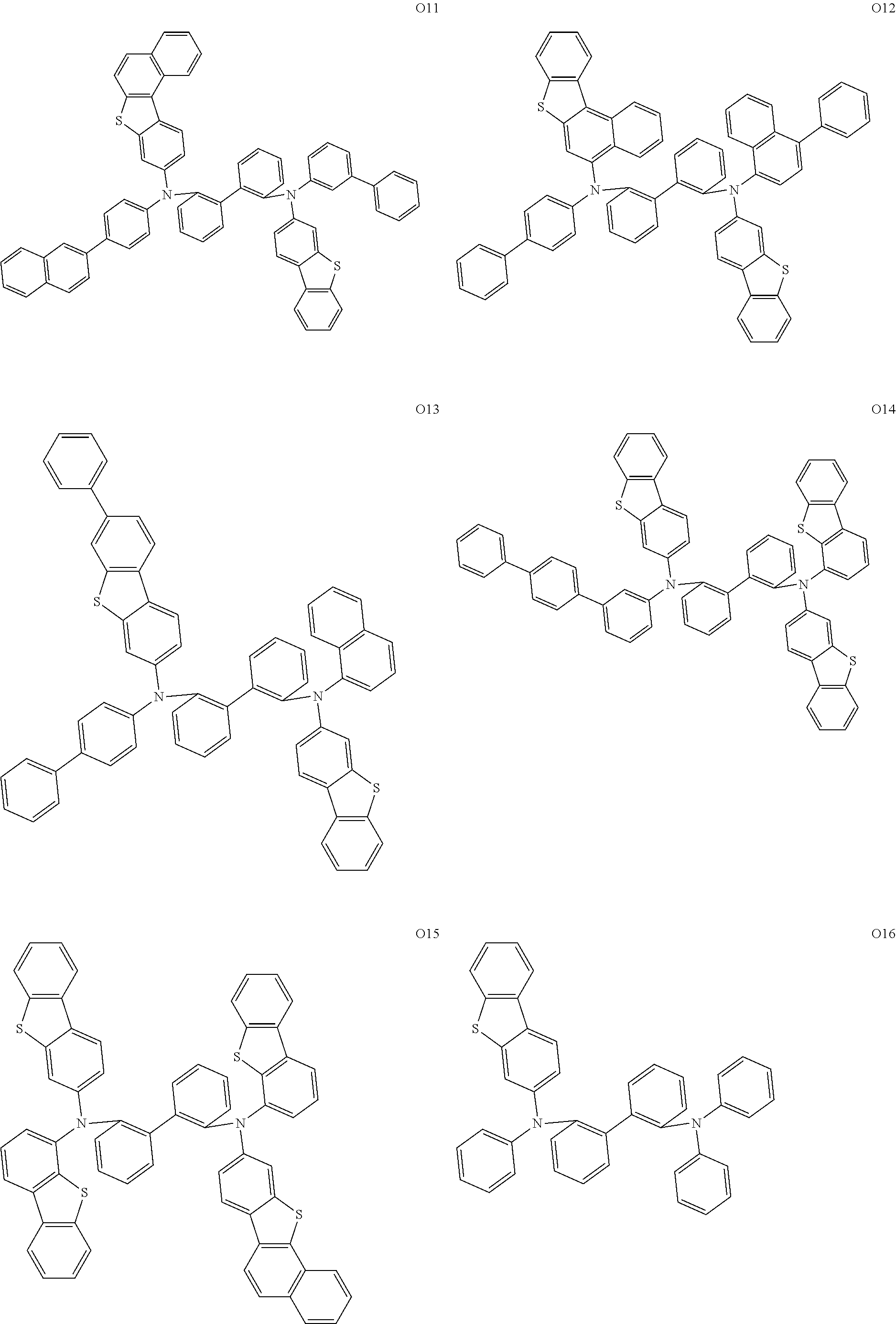

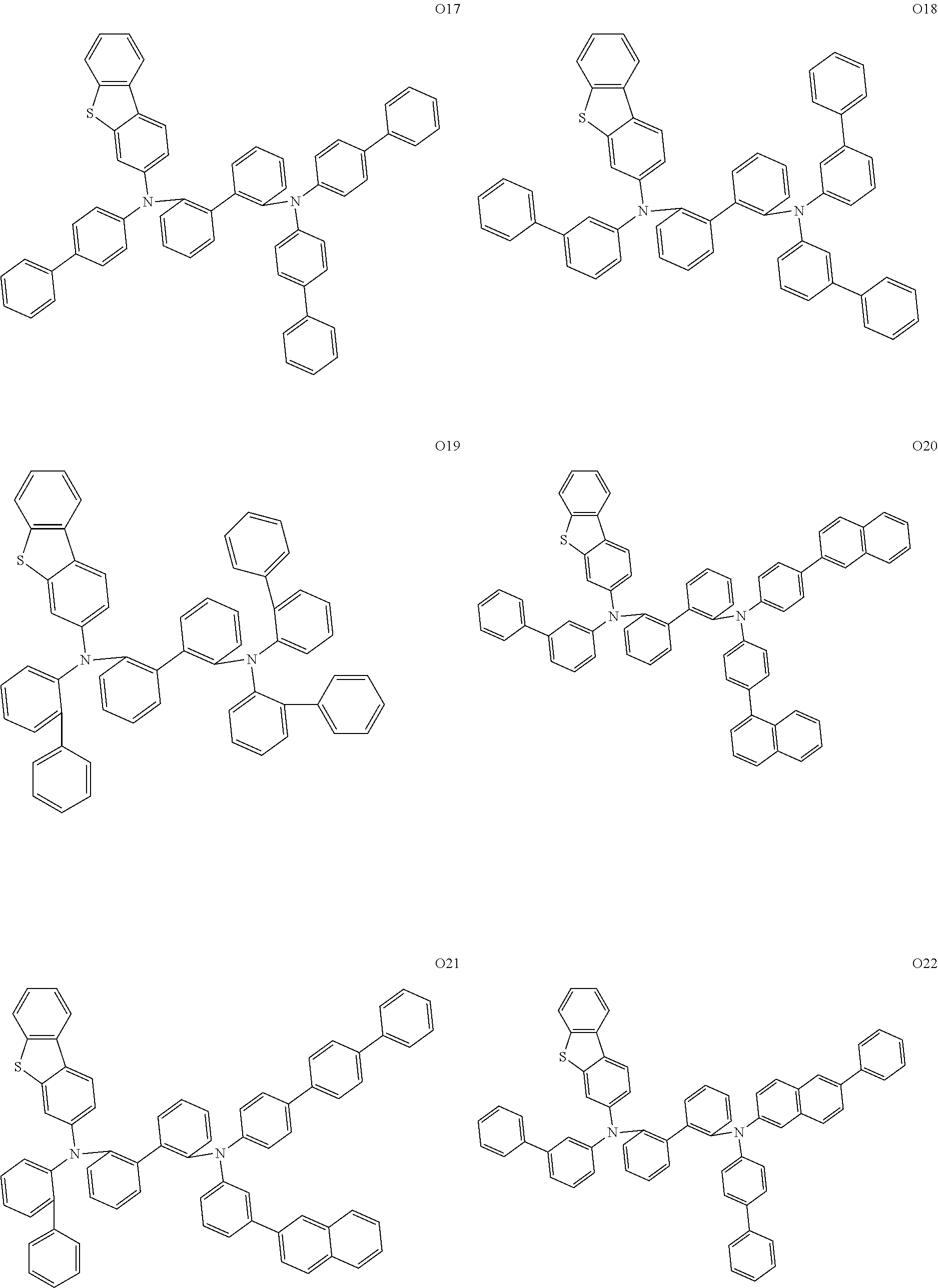

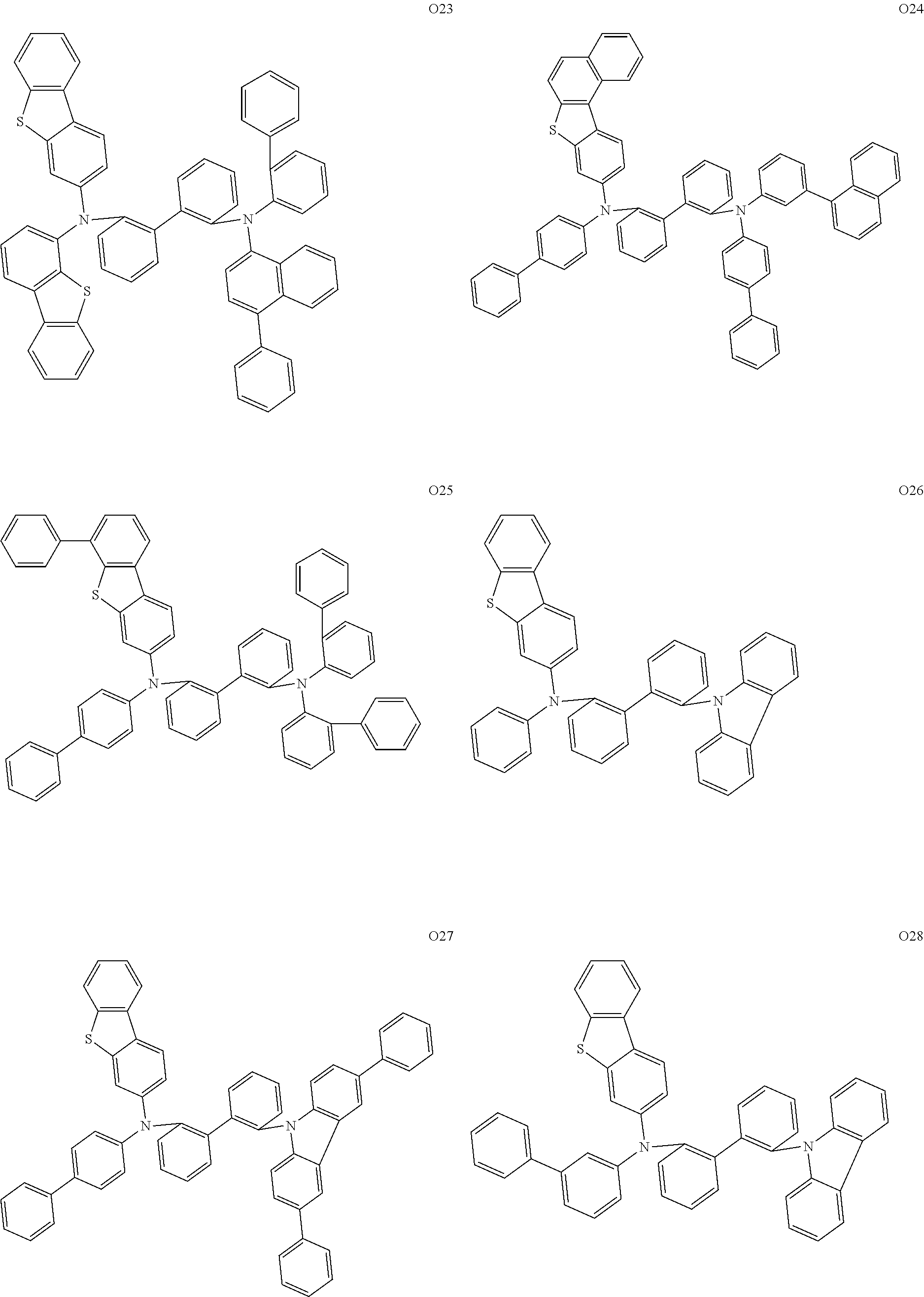

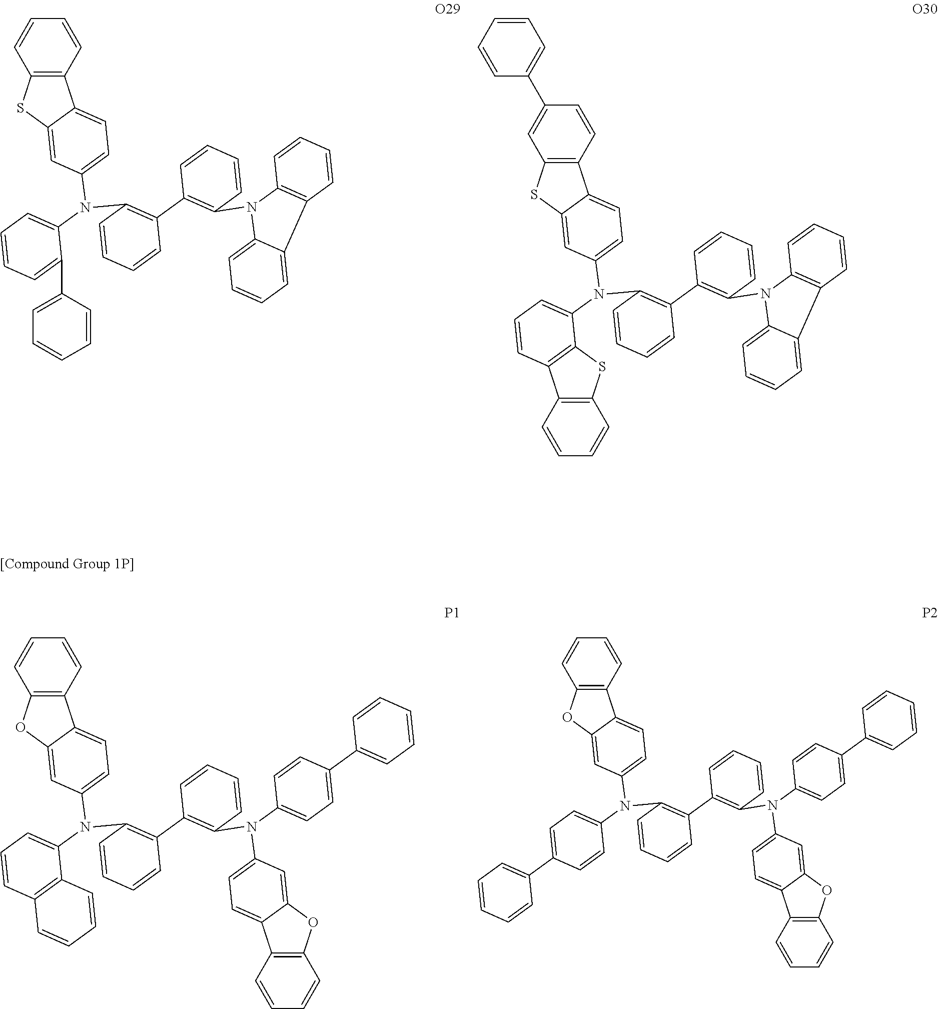 [SMILES: c1ccc(-c2ccccc2N(c2ccc3c(c2)sc2ccccc23)c2ccccc2-c2ccccc2-n2c3ccccc3c3ccccc32)cc1 ; c1ccc(-c2ccc3c(c2)sc2cc(N(c4ccccc4-c4ccccc4-n4c5ccccc5c5ccccc54)c4cccc5c4sc4ccccc45)ccc23)cc1 ; c1ccc(-c2ccc(N(c3ccc4c(c3)oc3ccccc34)c3ccccc3-c3ccccc3N(c3ccc4c(c3)oc3ccccc34)c3cccc4ccccc34)cc2)cc1 ; c1ccc(-c2ccc(N(c3ccc4c(c3)oc3ccccc34)c3ccccc3-c3ccccc3N(c3ccc(-c4ccccc4)cc3)c3ccc4c(c3)oc3ccccc34)cc2)cc1]

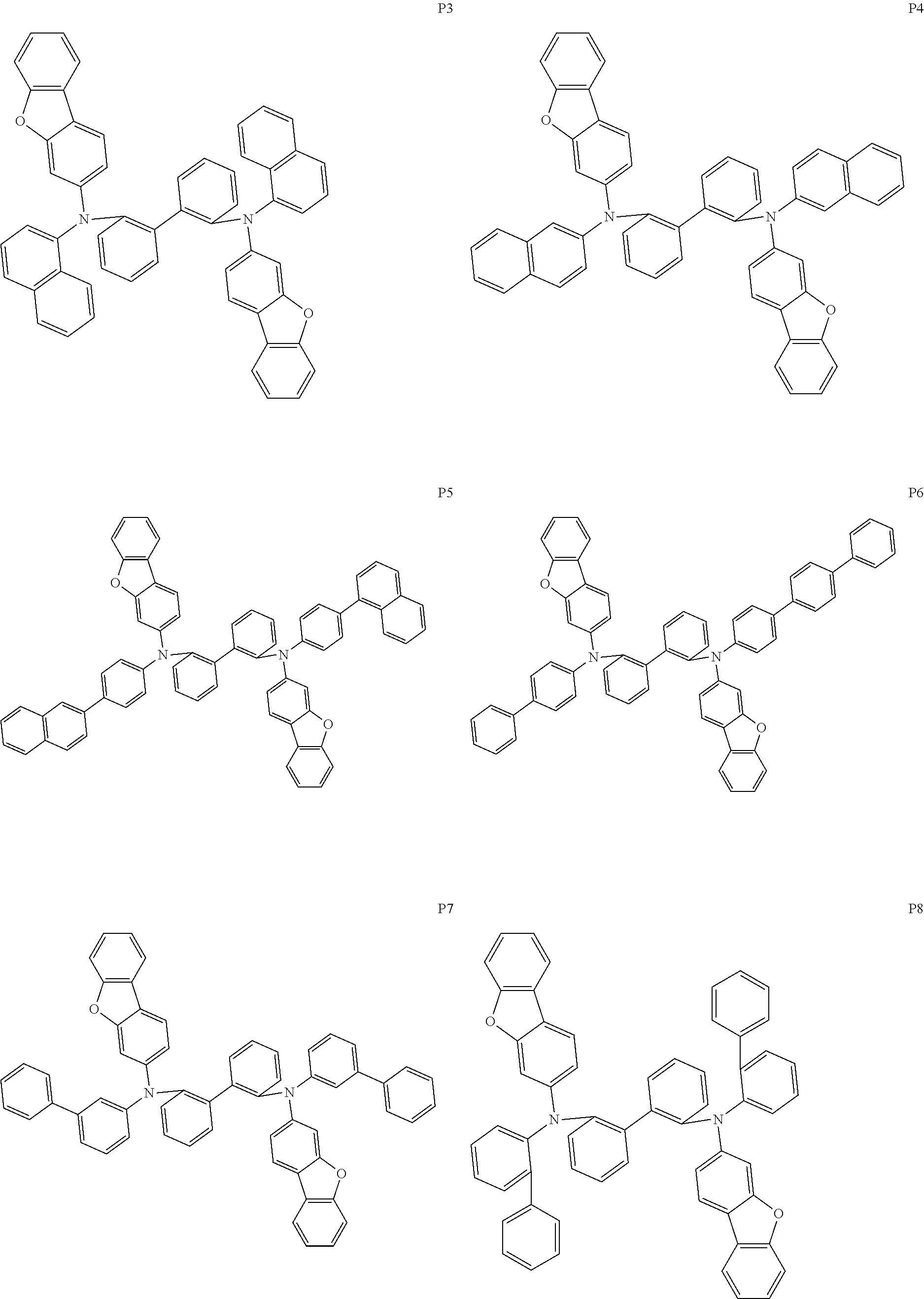

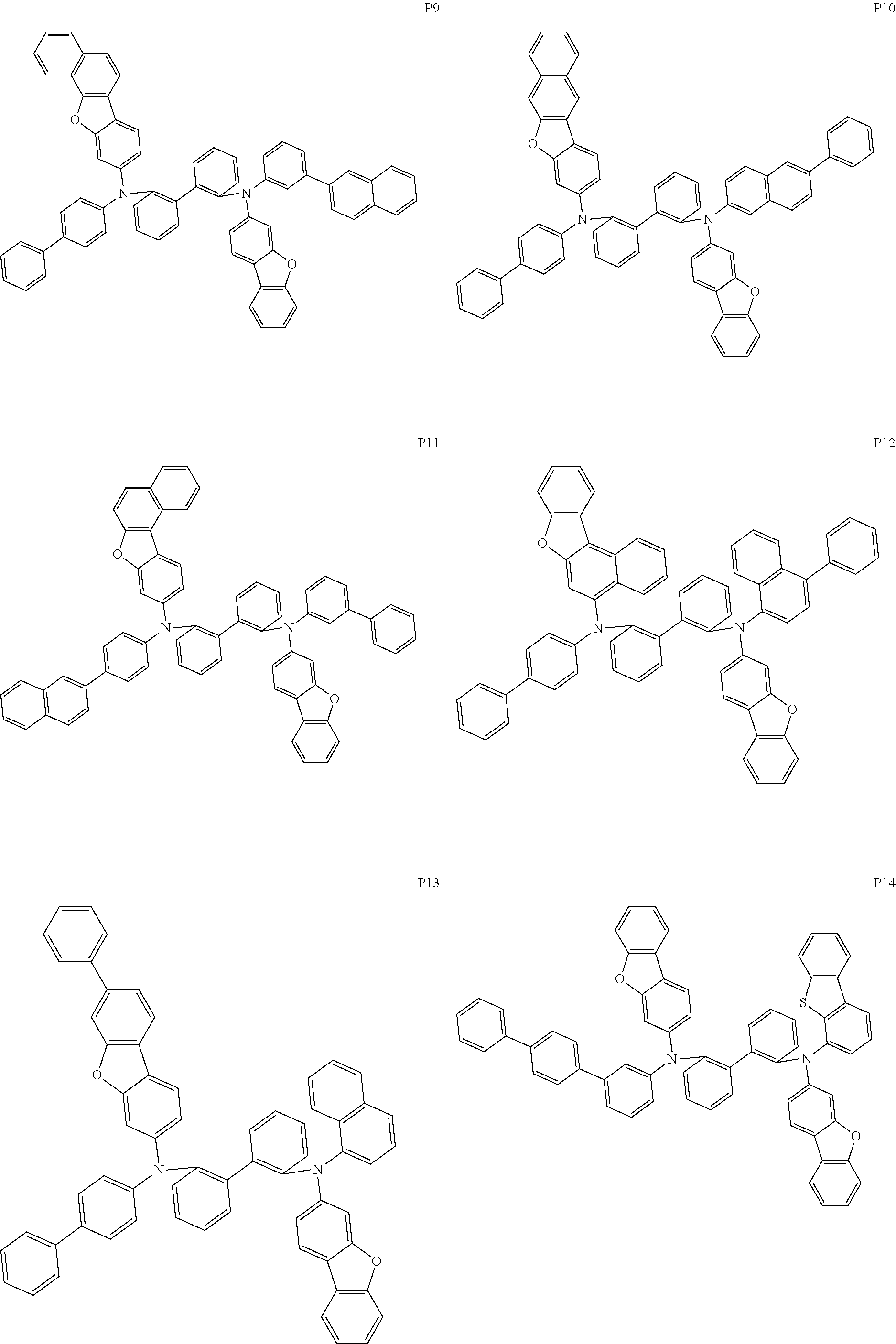

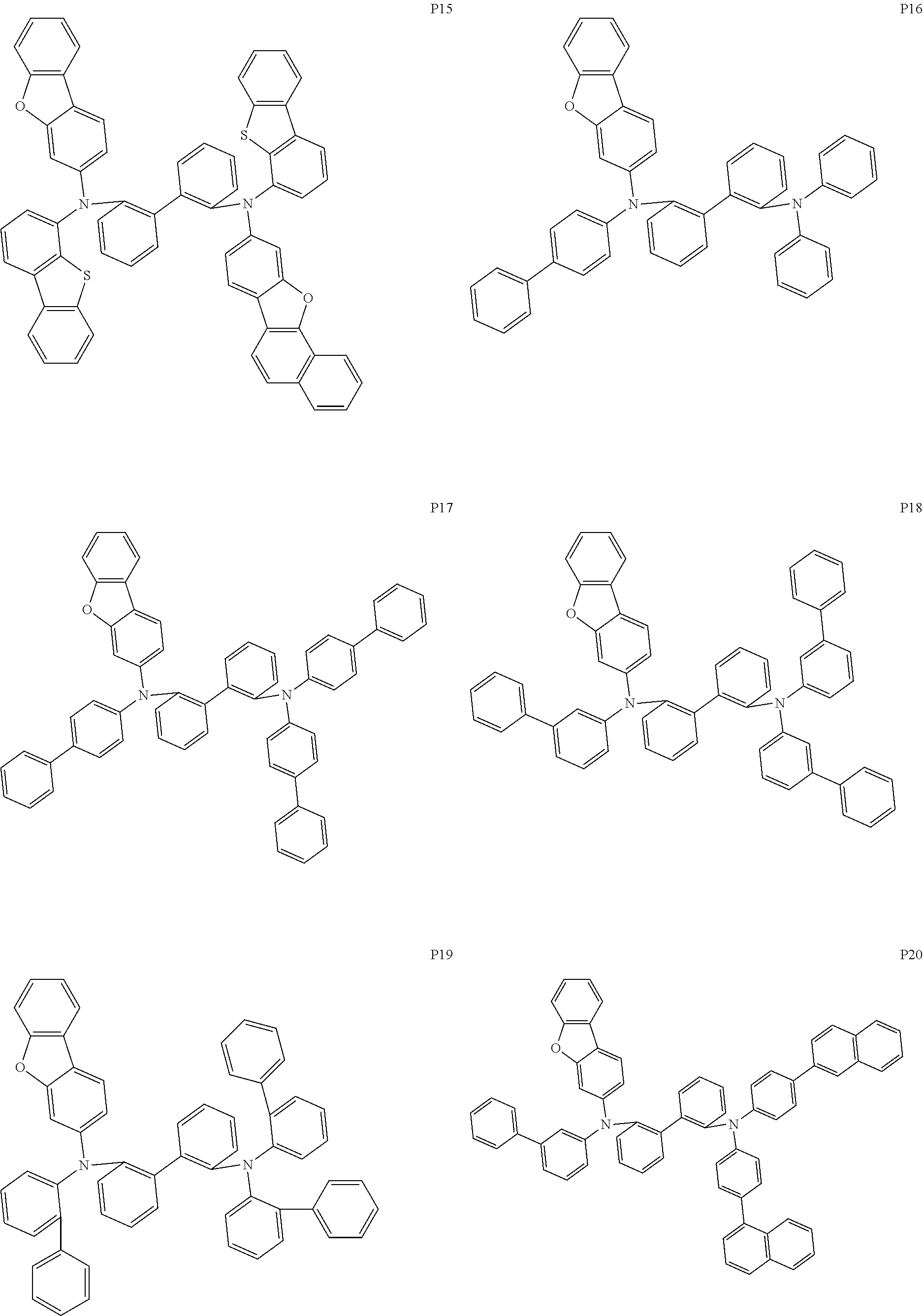

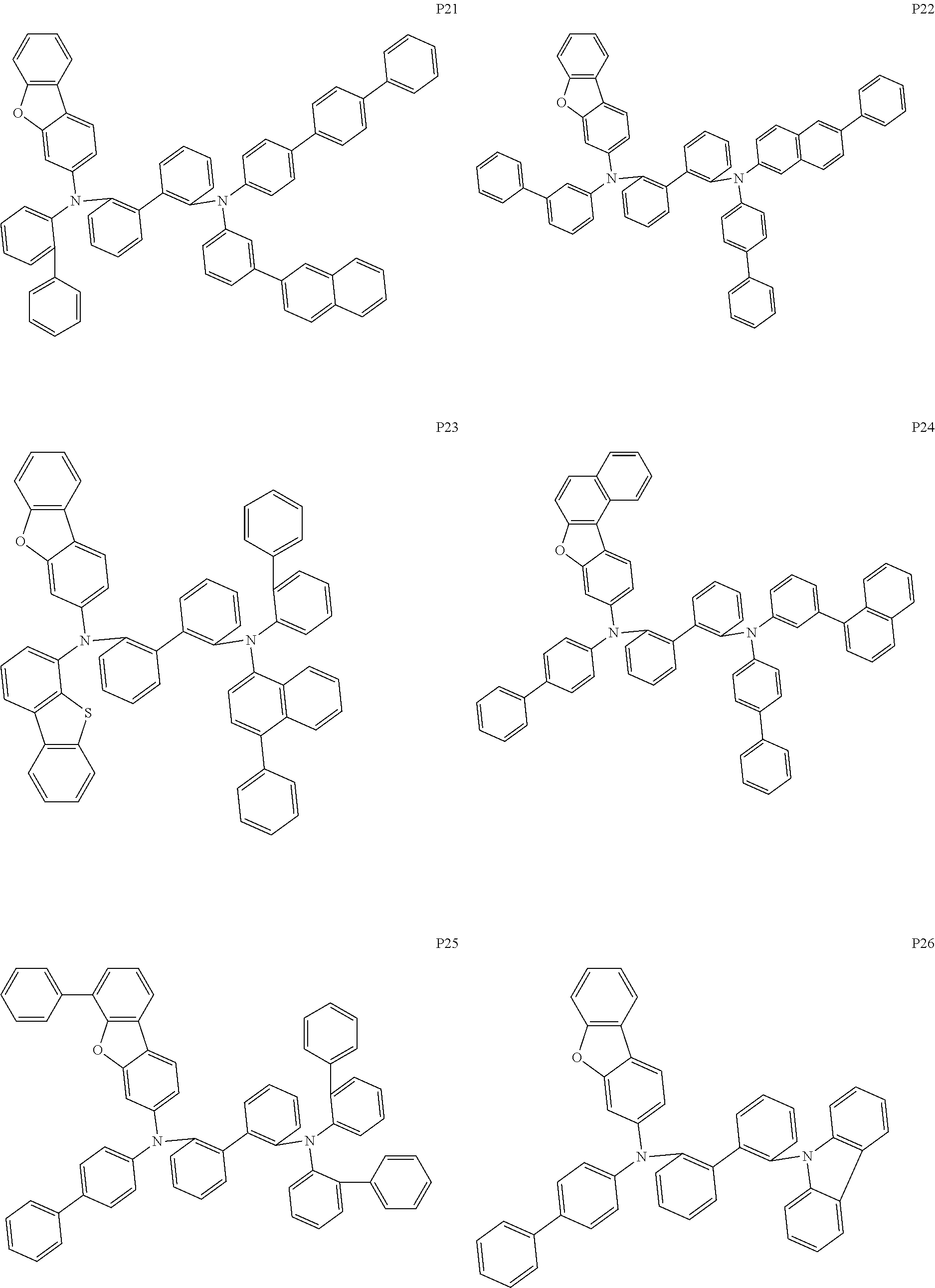

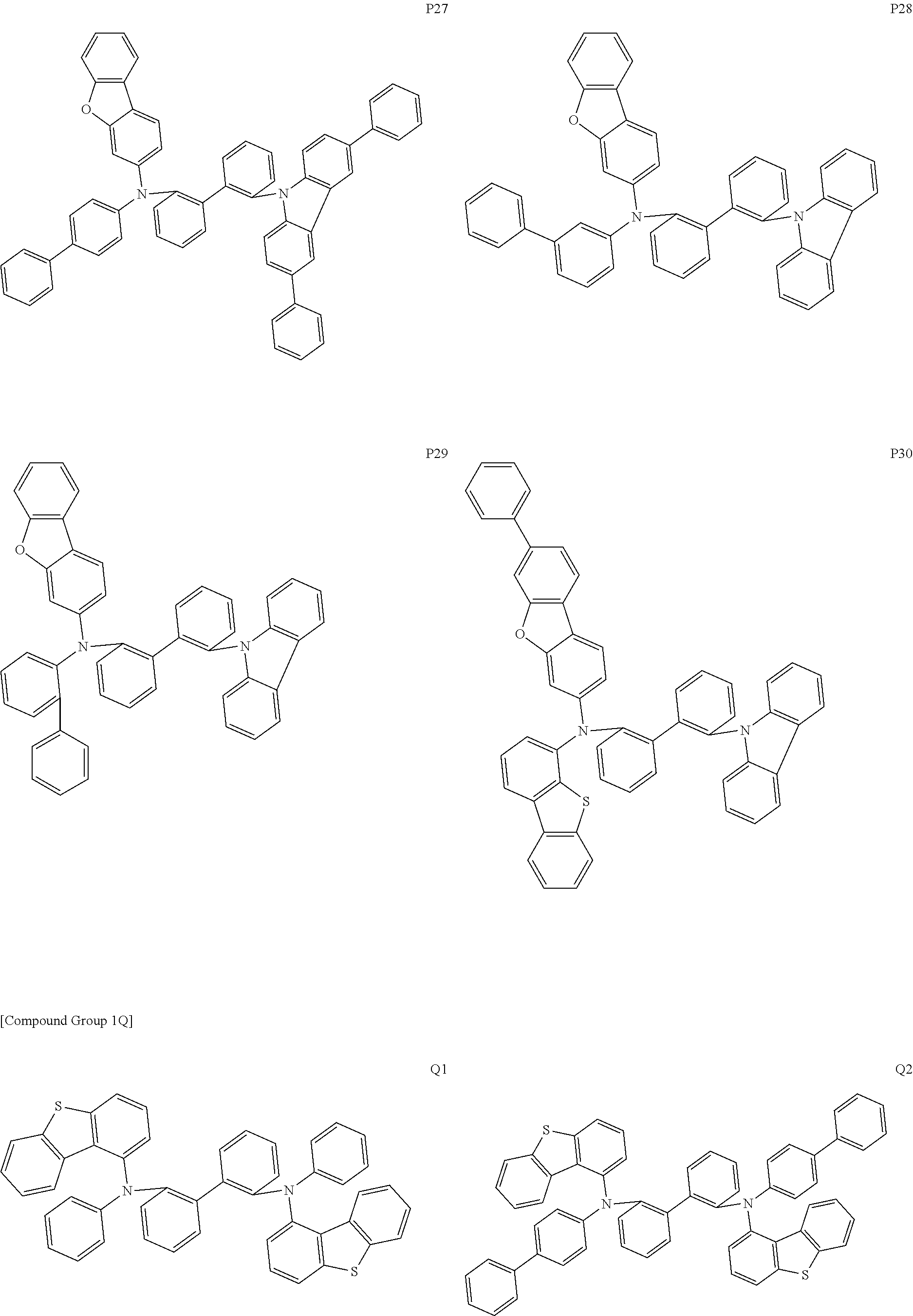

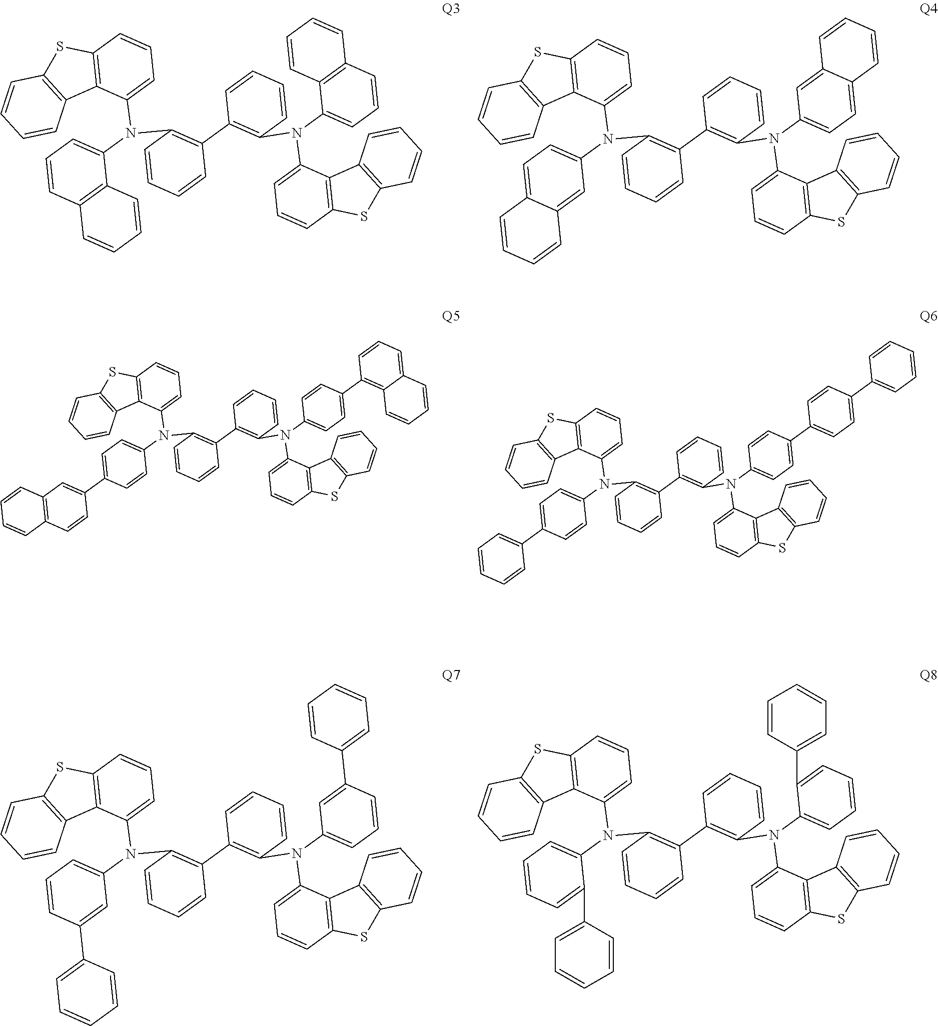

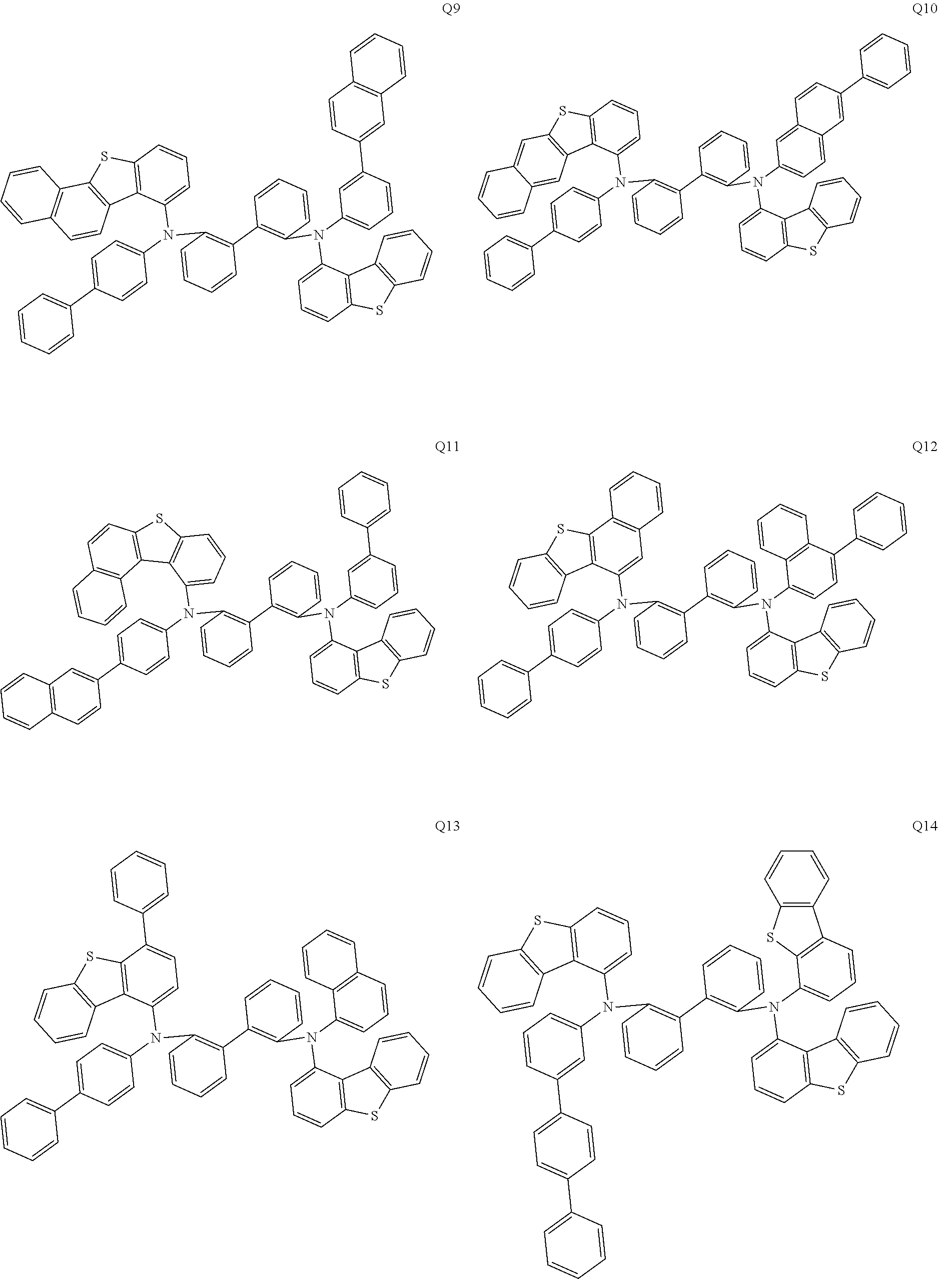

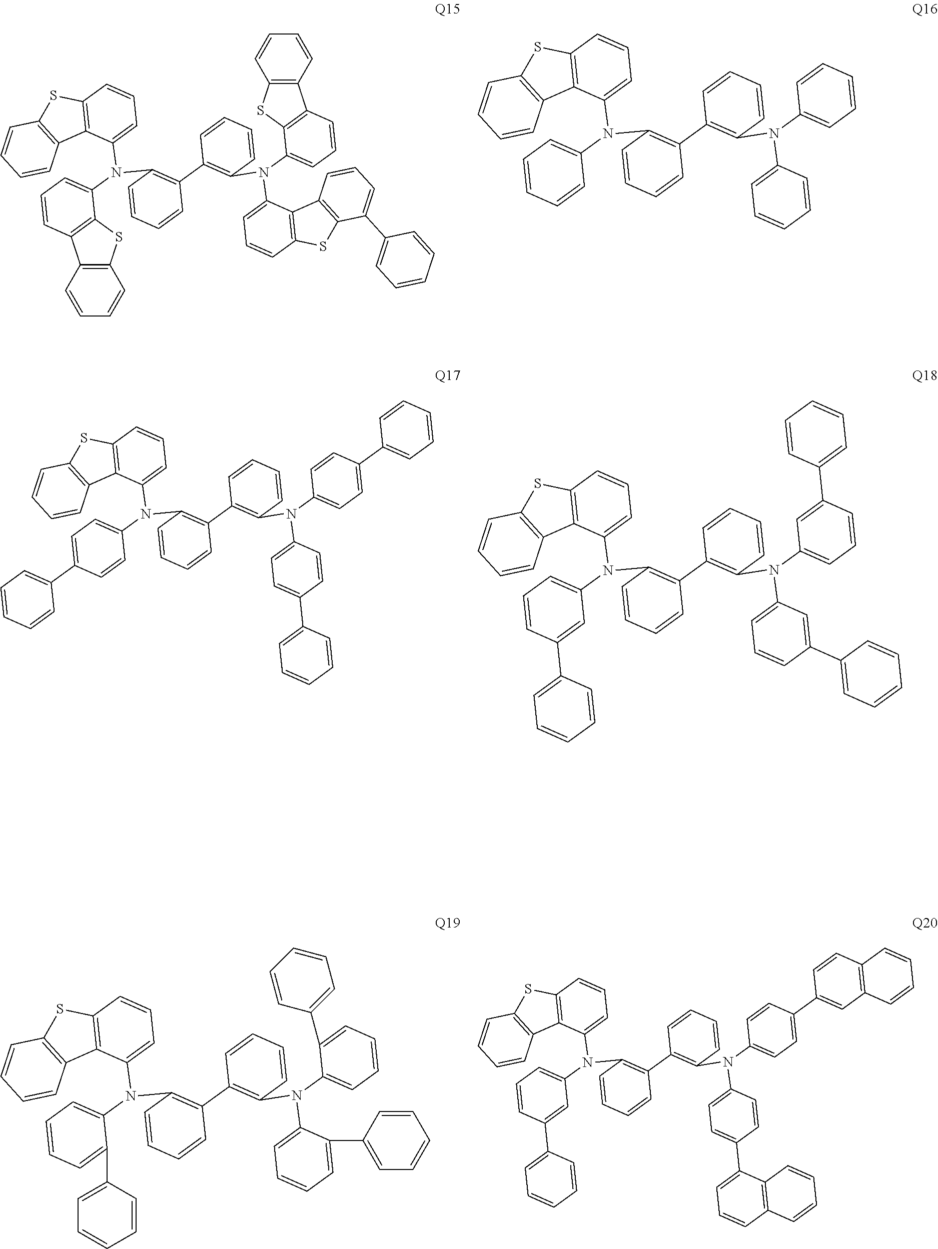

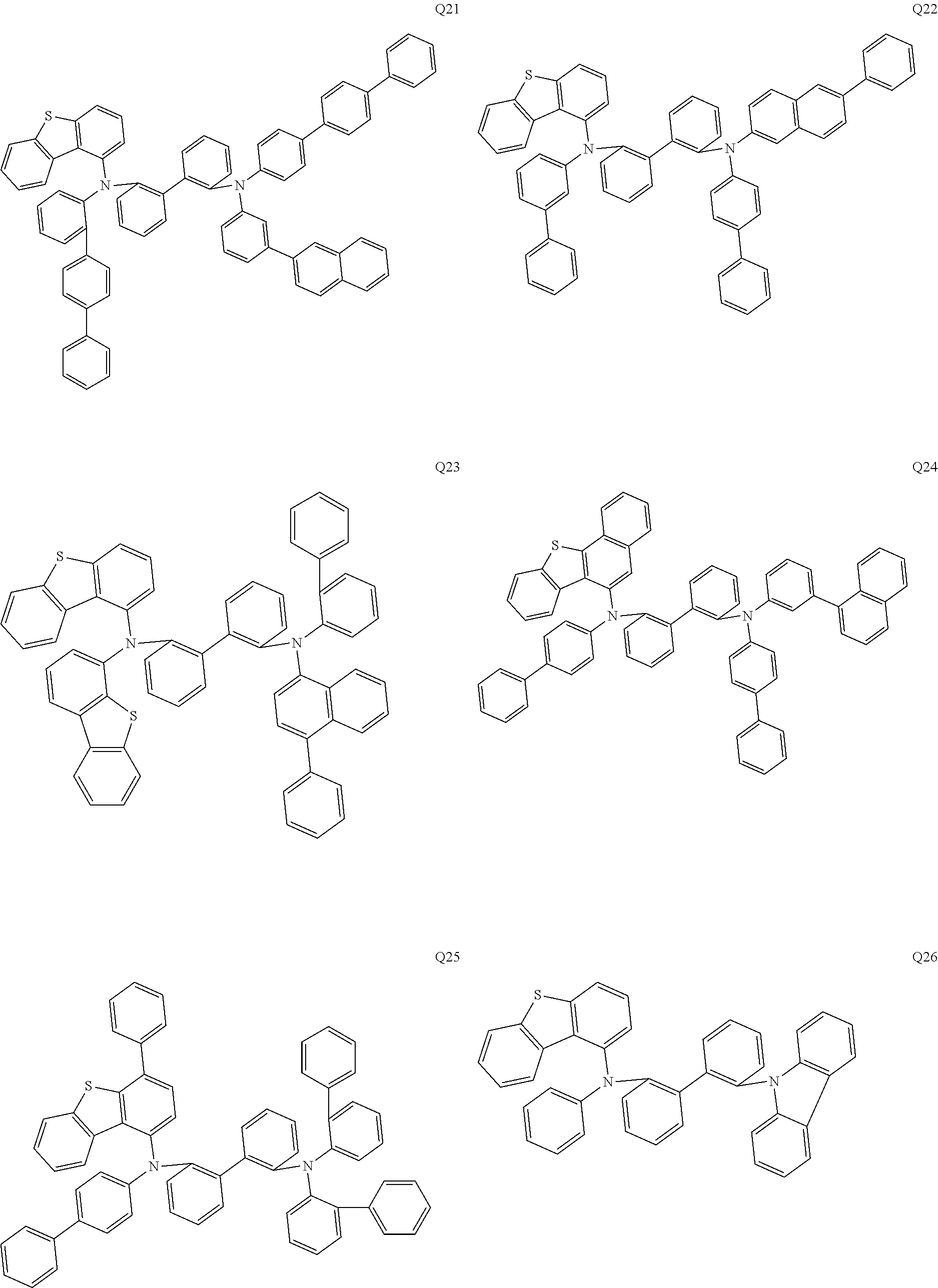

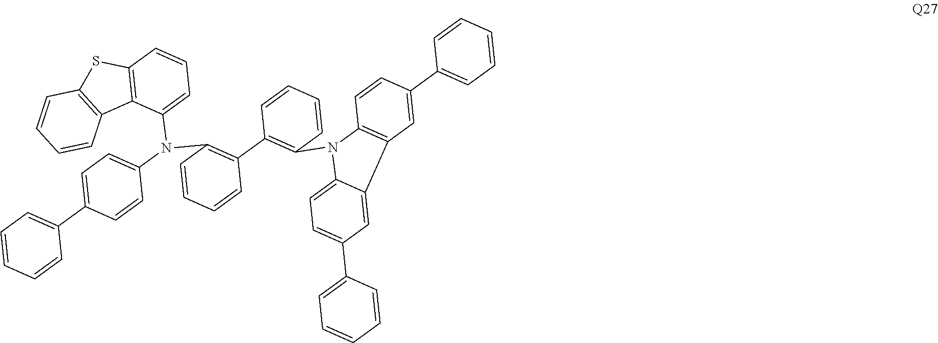 [SMILES: c1ccc(-c2ccc(N(c3ccccc3-c3ccccc3-n3c4ccc(-c5ccccc5)cc4c4cc(-c5ccccc5)ccc43)c3cccc4sc5ccccc5c34)cc2)cc1]

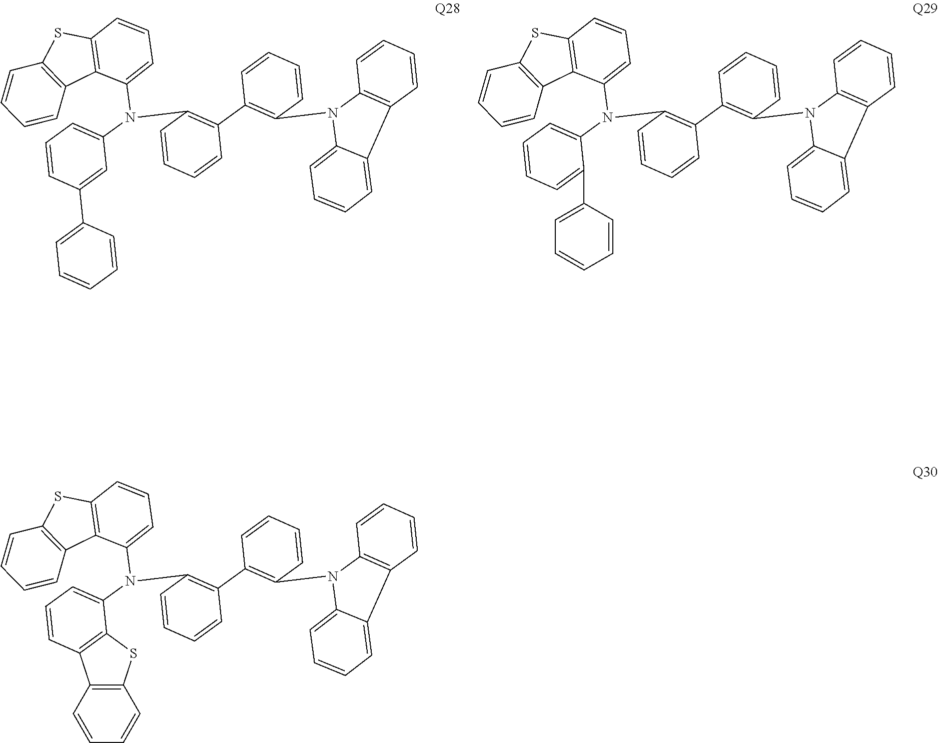 [SMILES: c1ccc(-c2cccc(N(c3ccccc3-c3ccccc3-n3c4ccccc4c4ccccc43)c3cccc4sc5ccccc5c34)c2)cc1 ; c1ccc(-c2ccccc2N(c2ccccc2-c2ccccc2-n2c3ccccc3c3ccccc32)c2cccc3sc4ccccc4c23)cc1 ; c1ccc(N(c2cccc3c2sc2ccccc23)c2cccc3sc4ccccc4c23)c(-c2ccccc2-n2c3ccccc3c3ccccc32)c1]

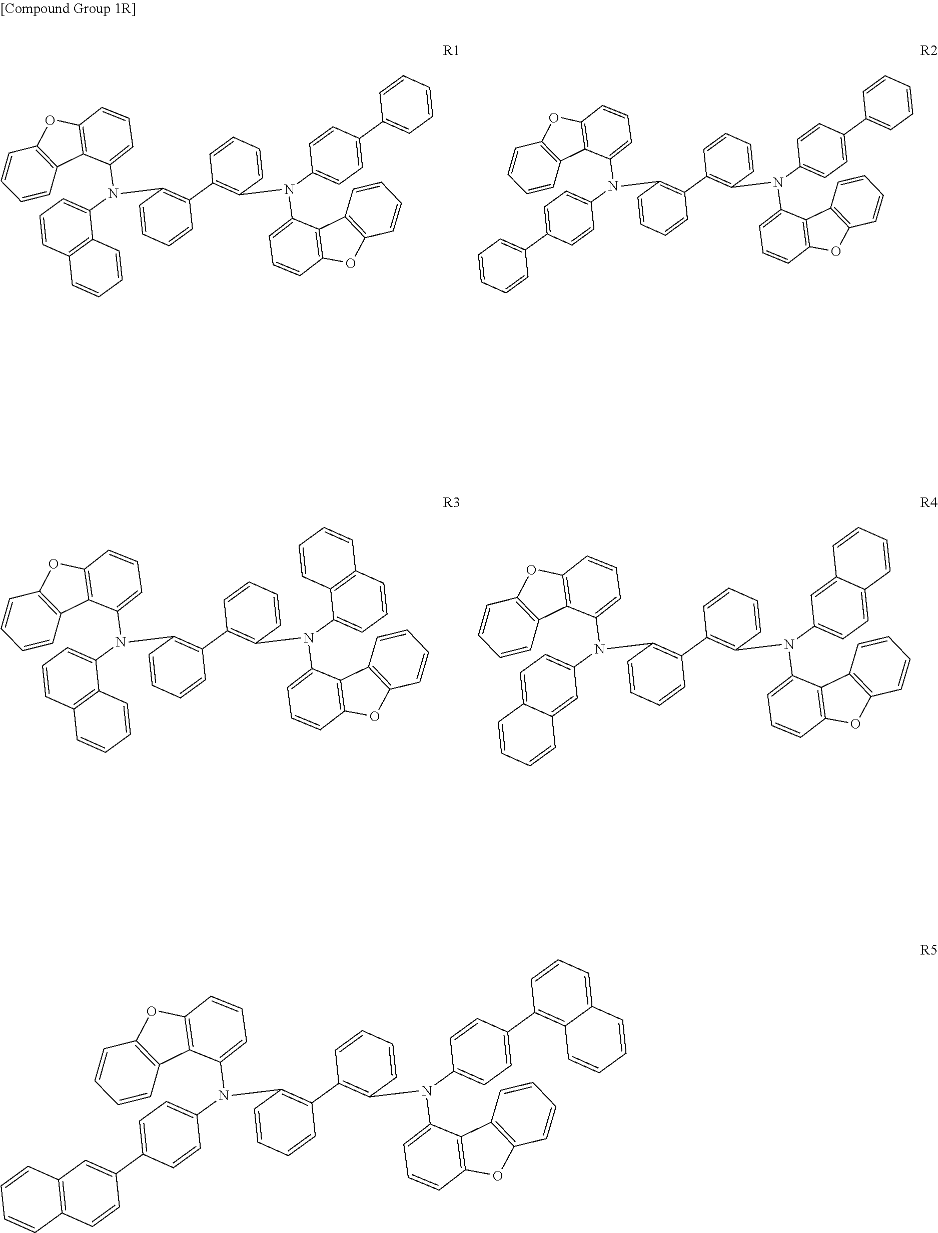

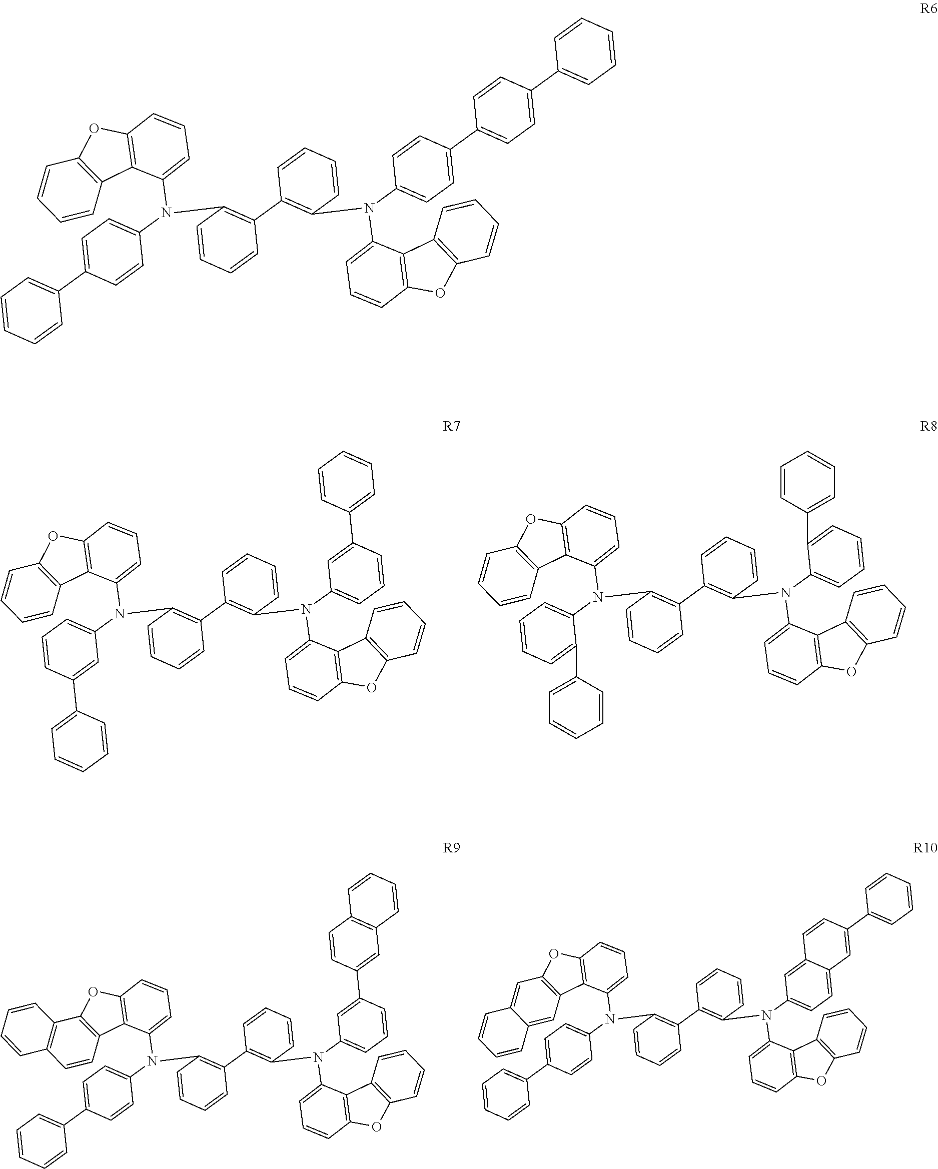

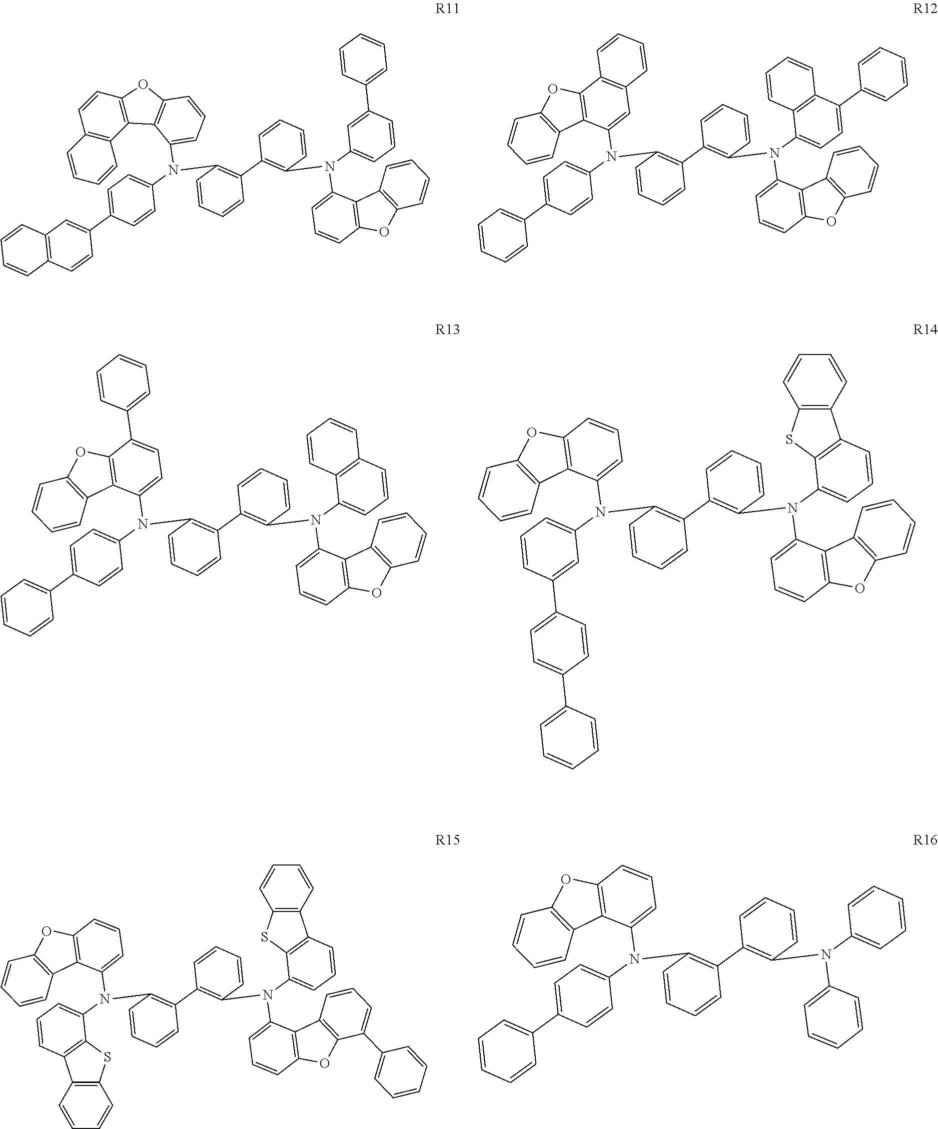

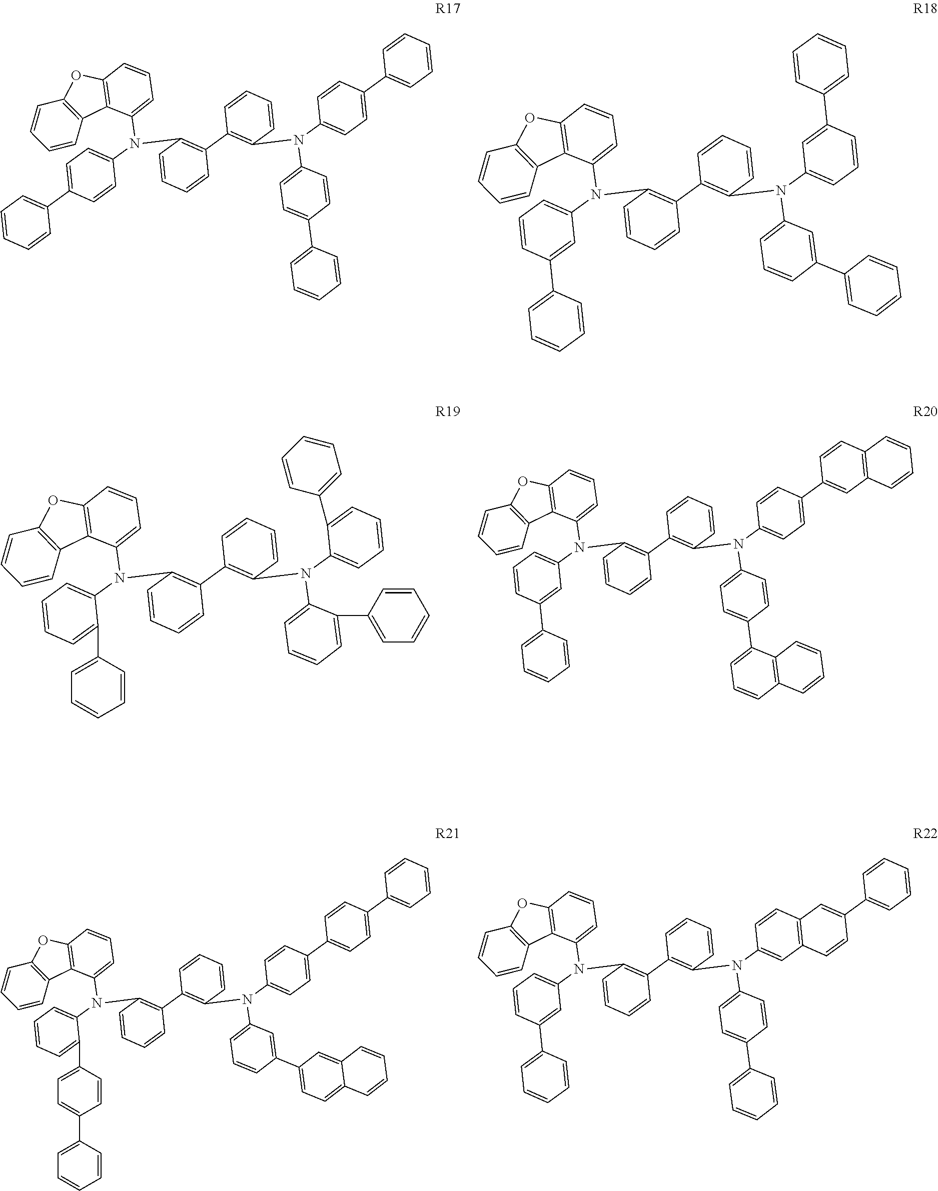

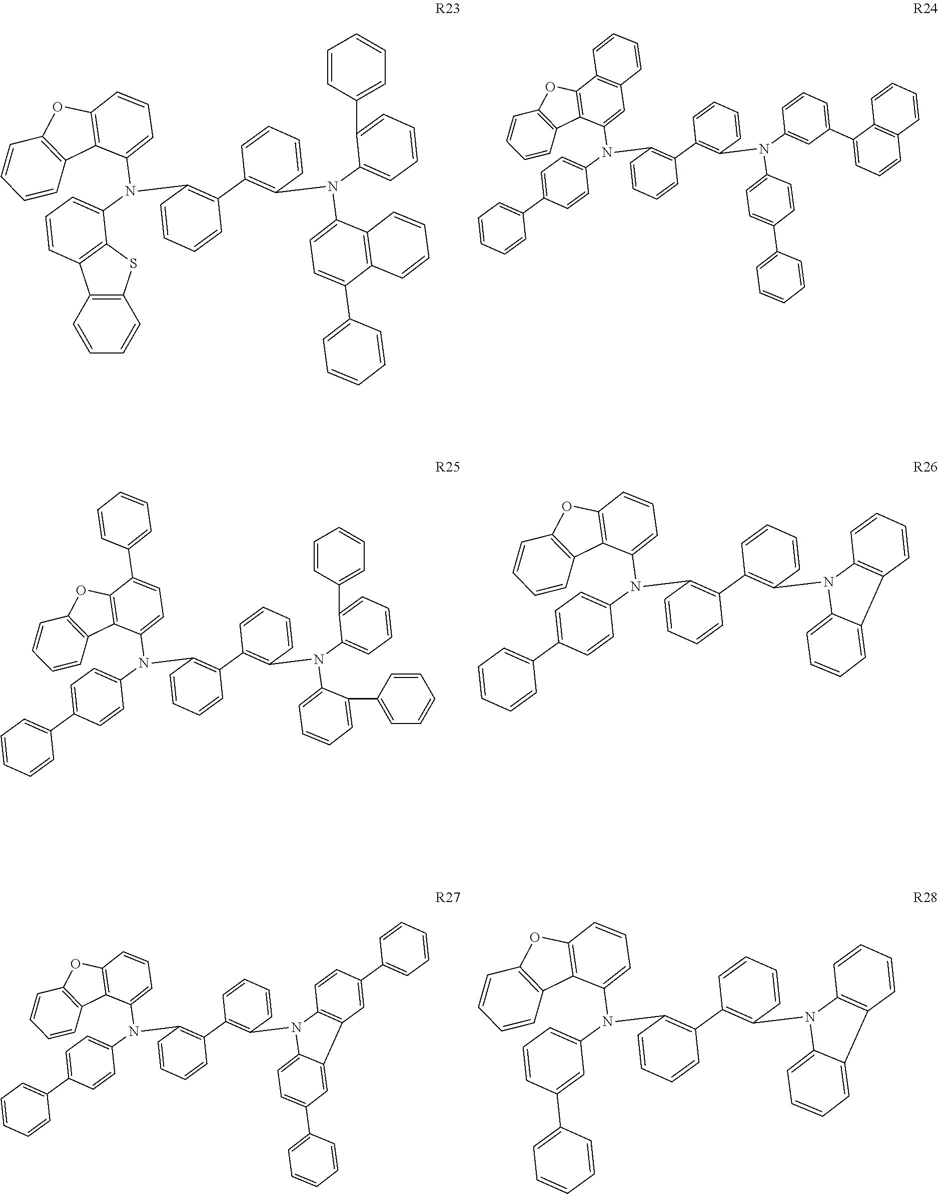

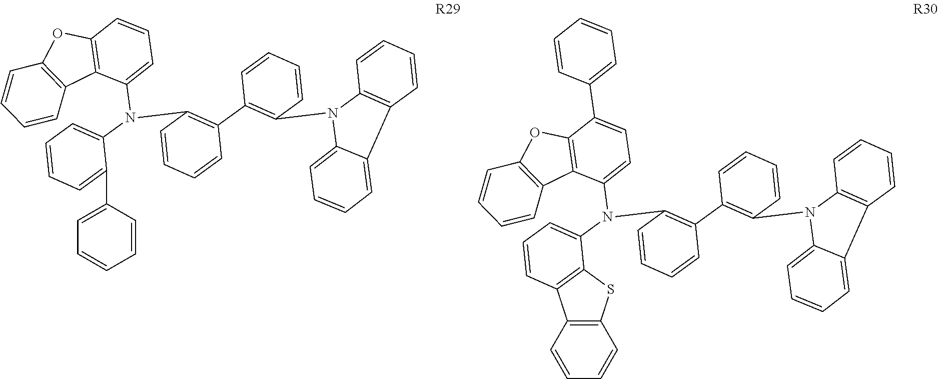 [SMILES: c1ccc(-c2ccccc2N(c2ccccc2-c2ccccc2-n2c3ccccc3c3ccccc32)c2cccc3oc4ccccc4c23)cc1 ; c1ccc(-c2ccc(N(c3ccccc3-c3ccccc3-n3c4ccccc4c4ccccc43)c3cccc4c3sc3ccccc34)c3c2oc2ccccc23)cc1]

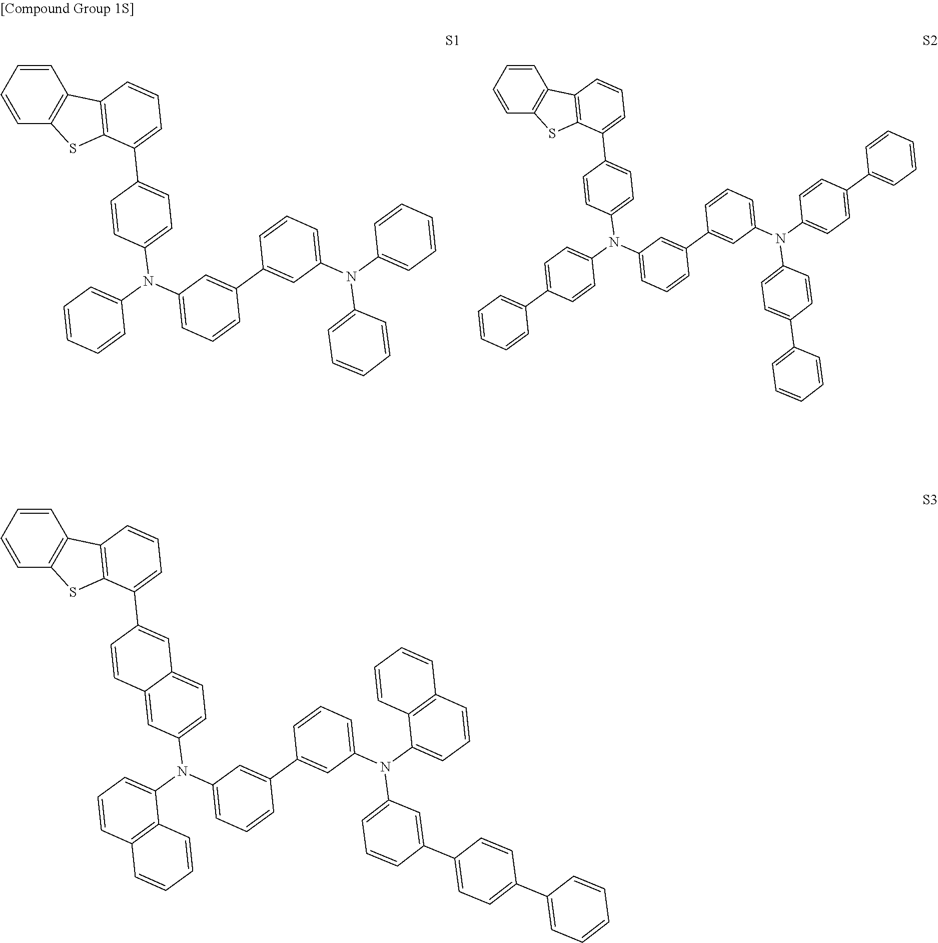 [SMILES: c1ccc(N(c2ccccc2)c2cccc(-c3cccc(N(c4ccccc4)c4ccc(-c5cccc6c5sc5ccccc56)cc4)c3)c2)cc1 ; c1ccc(-c2ccc(N(c3ccc(-c4ccccc4)cc3)c3cccc(-c4cccc(N(c5ccc(-c6ccccc6)cc5)c5ccc(-c6cccc7c6sc6ccccc67)cc5)c4)c3)cc2)cc1 ; c1ccc(-c2ccc(-c3cccc(N(c4cccc(-c5cccc(N(c6ccc7cc(-c8cccc9c8sc8ccccc89)ccc7c6)c6cccc7ccccc67)c5)c4)c4cccc5ccccc45)c3)cc2)cc1]

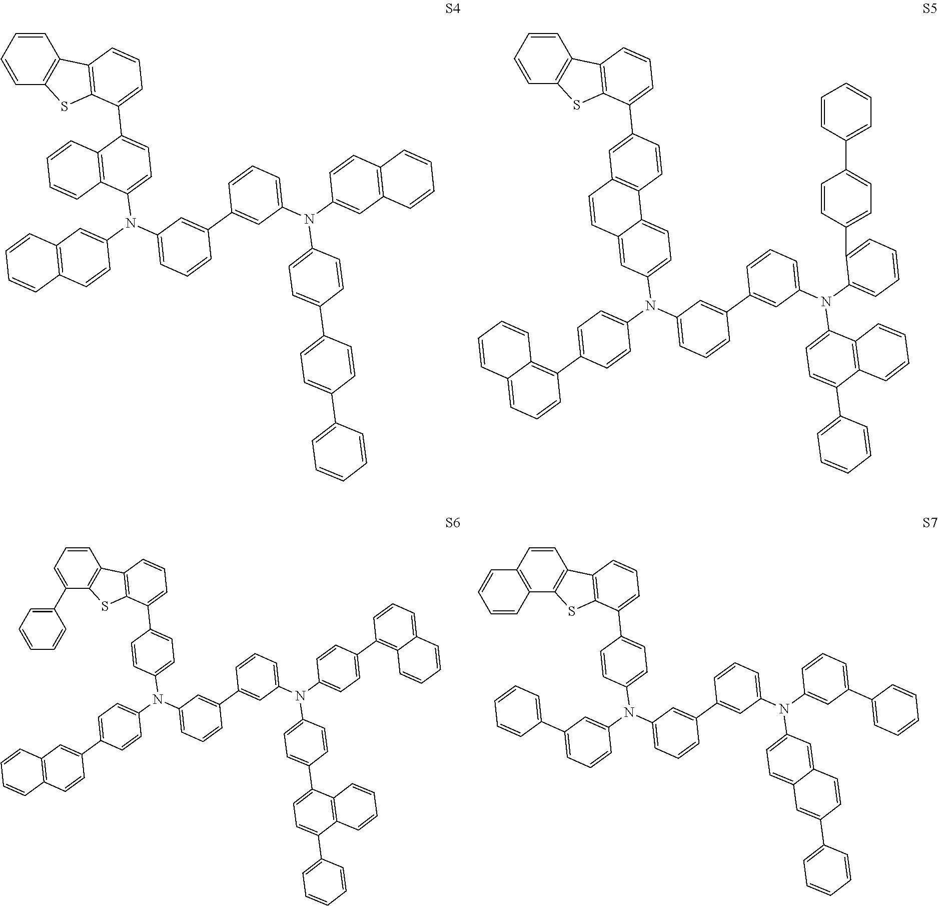

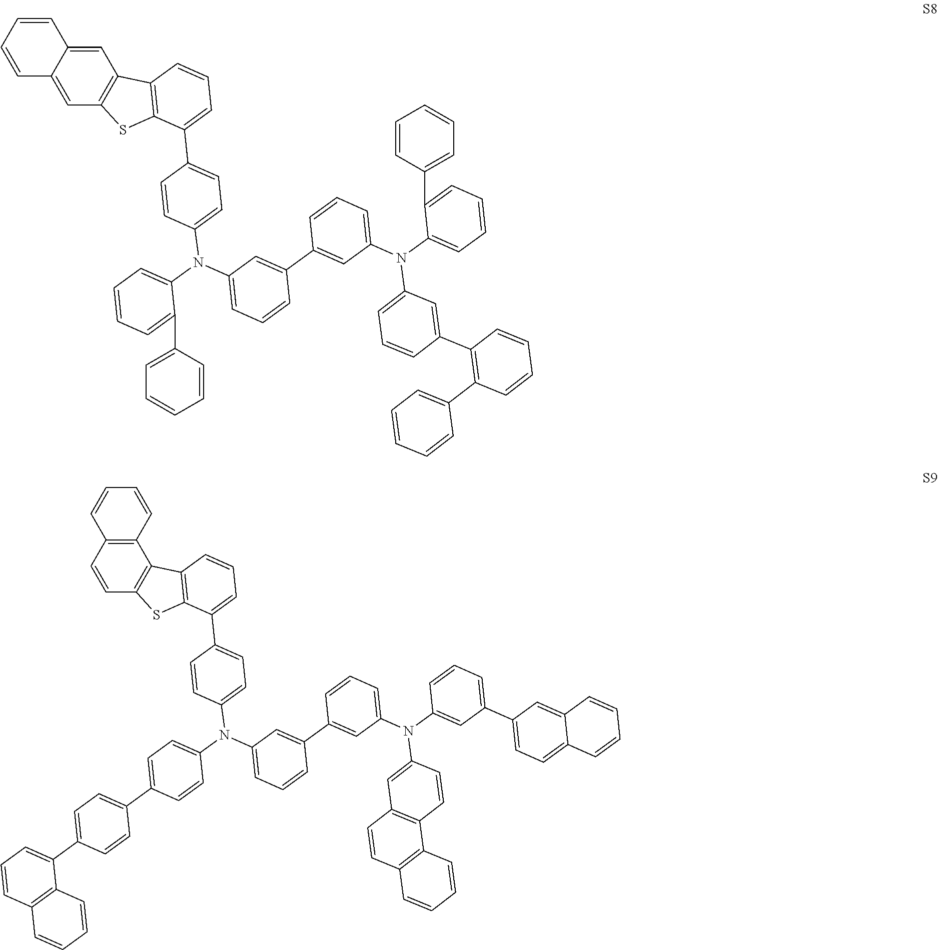 [SMILES: c1ccc(-c2ccccc2-c2cccc(N(c3cccc(-c4cccc(N(c5ccc(-c6cccc7c6sc6cc8ccccc8cc67)cc5)c5ccccc5-c5ccccc5)c4)c3)c3ccccc3-c3ccccc3)c2)cc1 ; c1cc(-c2cccc(N(c3cccc(-c4ccc5ccccc5c4)c3)c3ccc4c(ccc5ccccc54)c3)c2)cc(N(c2ccc(-c3ccc(-c4cccc5ccccc45)cc3)cc2)c2ccc(-c3cccc4c3sc3ccc5ccccc5c34)cc2)c1]

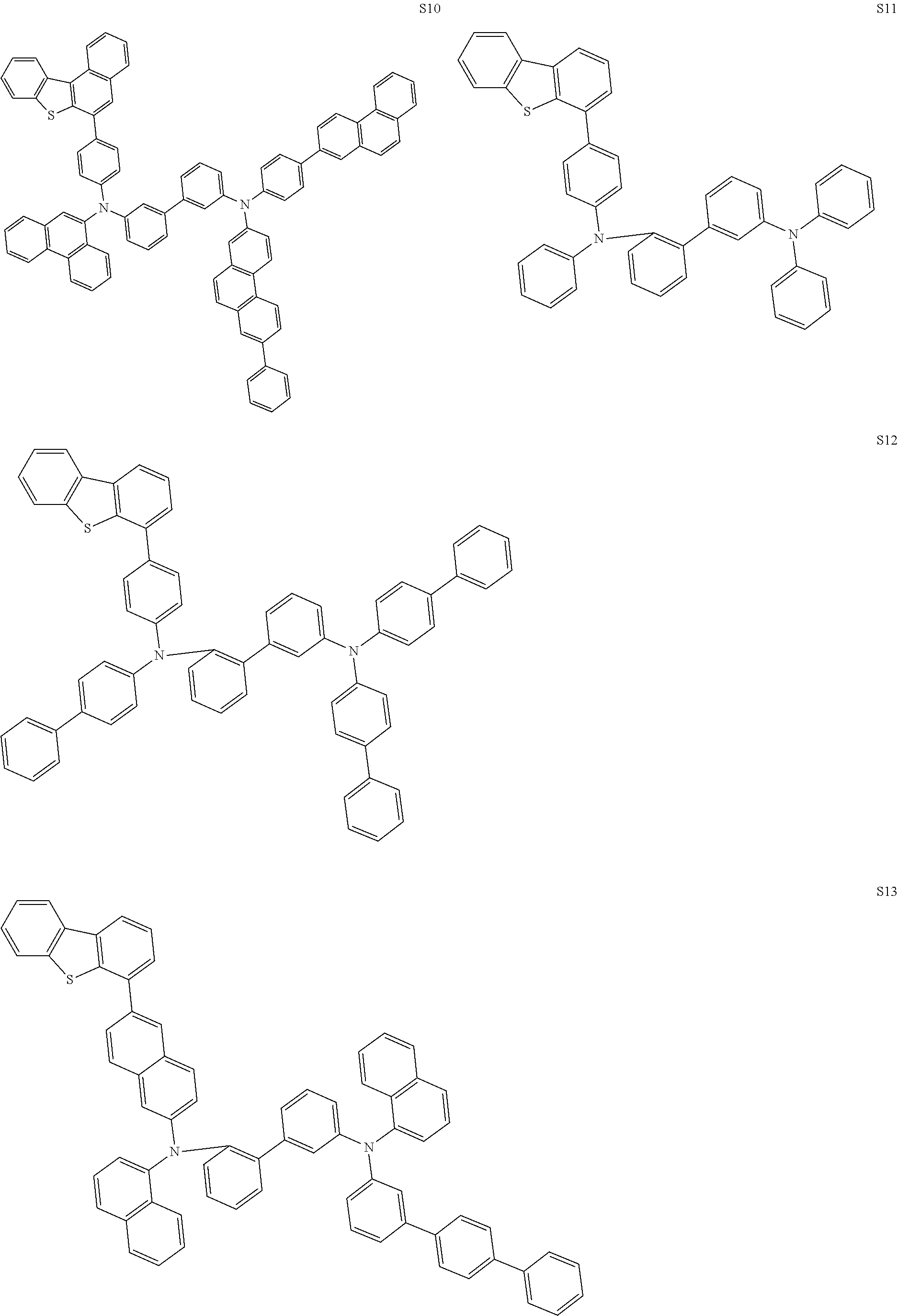 [SMILES: c1ccc(-c2ccc3c(ccc4cc(N(c5ccc(-c6ccc7c(ccc8ccccc87)c6)cc5)c5cccc(-c6cccc(N(c7ccc(-c8cc9ccccc9c9c8sc8ccccc89)cc7)c7cc8ccccc8c8ccccc78)c6)c5)ccc43)c2)cc1 ; c1ccc(N(c2ccccc2)c2cccc(-c3ccccc3N(c3ccccc3)c3ccc(-c4cccc5c4sc4ccccc45)cc3)c2)cc1 ; c1ccc(-c2ccc(N(c3ccc(-c4ccccc4)cc3)c3cccc(-c4ccccc4N(c4ccc(-c5ccccc5)cc4)c4ccc(-c5cccc6c5sc5ccccc56)cc4)c3)cc2)cc1 ; c1ccc(-c2ccc(-c3cccc(N(c4cccc(-c5ccccc5N(c5ccc6cc(-c7cccc8c7sc7ccccc78)ccc6c5)c5cccc6ccccc56)c4)c4cccc5ccccc45)c3)cc2)cc1]

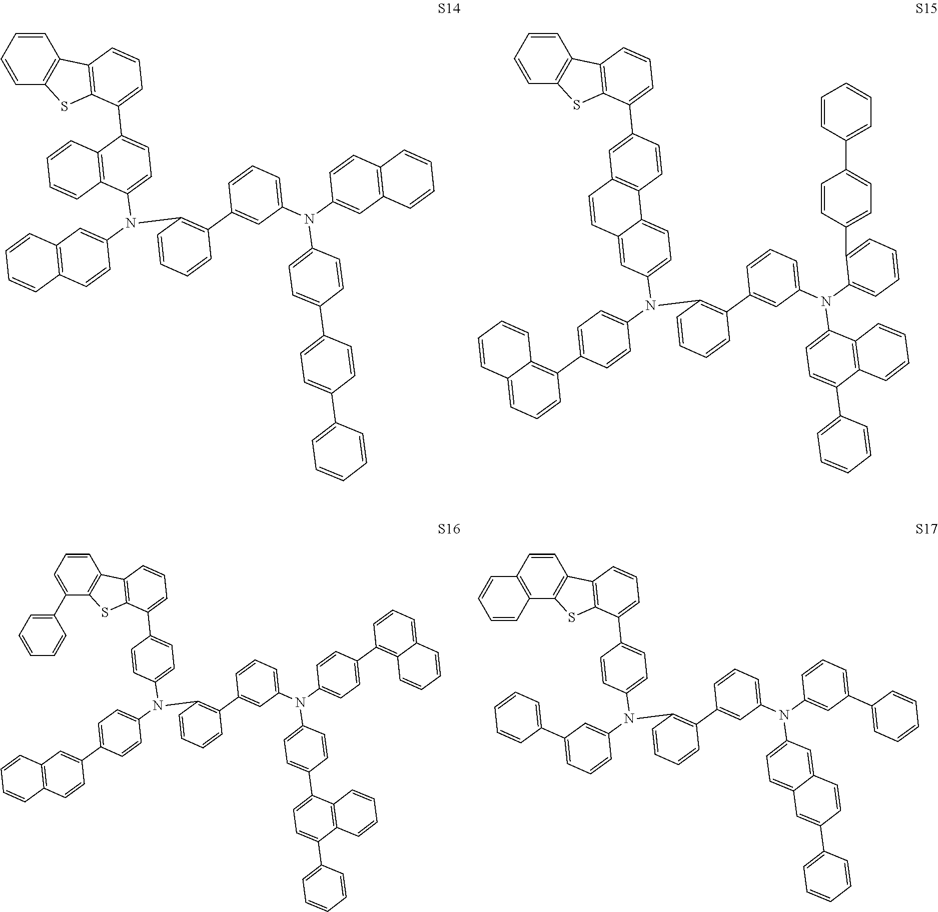

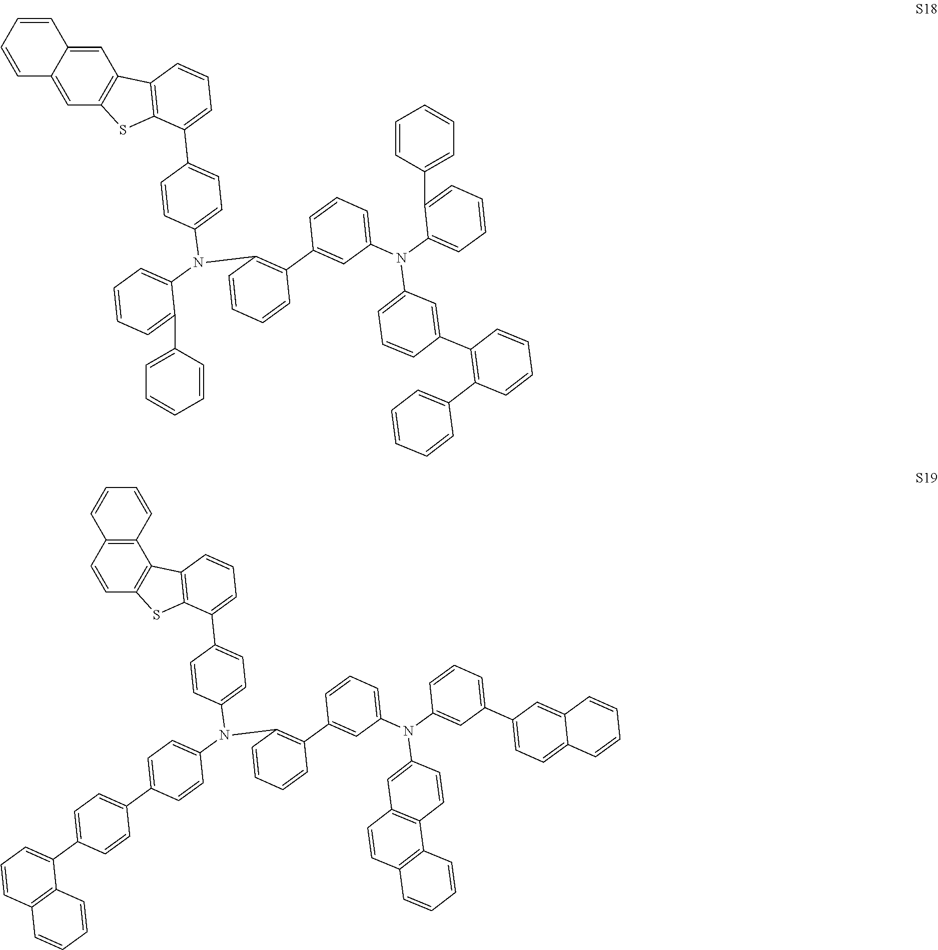 [SMILES: c1ccc(-c2ccccc2-c2cccc(N(c3cccc(-c4ccccc4N(c4ccc(-c5cccc6c5sc5cc7ccccc7cc56)cc4)c4ccccc4-c4ccccc4)c3)c3ccccc3-c3ccccc3)c2)cc1 ; c1cc(-c2ccc3ccccc3c2)cc(N(c2cccc(-c3ccccc3N(c3ccc(-c4ccc(-c5cccc6ccccc56)cc4)cc3)c3ccc(-c4cccc5c4sc4ccc6ccccc6c45)cc3)c2)c2ccc3c(ccc4ccccc43)c2)c1]

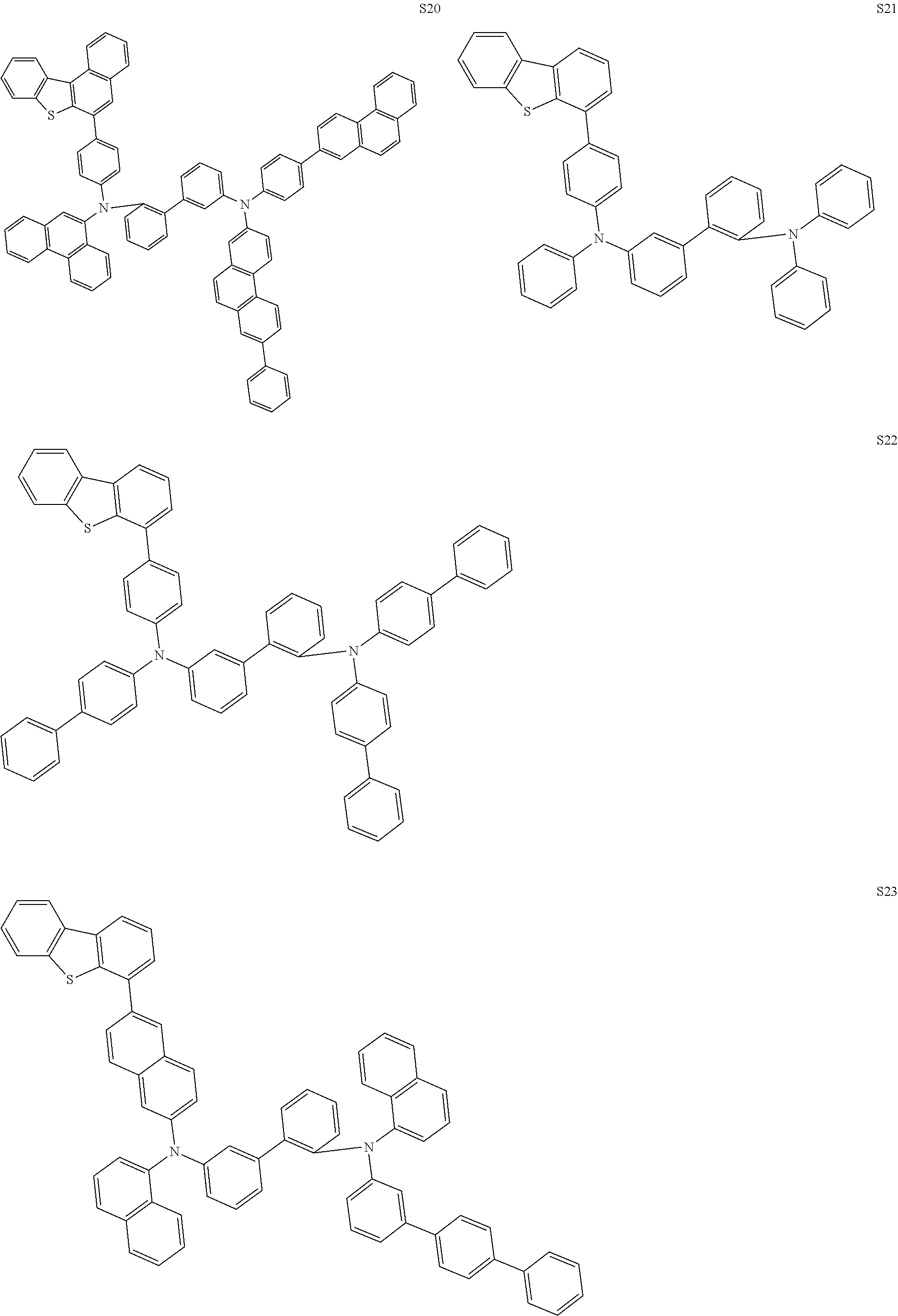 [SMILES: c1ccc(-c2ccc3c(ccc4cc(N(c5ccc(-c6ccc7c(ccc8ccccc87)c6)cc5)c5cccc(-c6ccccc6N(c6ccc(-c7cc8ccccc8c8c7sc7ccccc78)cc6)c6cc7ccccc7c7ccccc67)c5)ccc43)c2)cc1 ; c1ccc(N(c2ccc(-c3cccc4c3sc3ccccc34)cc2)c2cccc(-c3ccccc3N(c3ccccc3)c3ccccc3)c2)cc1 ; c1ccc(-c2ccc(N(c3ccc(-c4cccc5c4sc4ccccc45)cc3)c3cccc(-c4ccccc4N(c4ccc(-c5ccccc5)cc4)c4ccc(-c5ccccc5)cc4)c3)cc2)cc1 ; c1ccc(-c2ccc(-c3cccc(N(c4ccccc4-c4cccc(N(c5ccc6cc(-c7cccc8c7sc7ccccc78)ccc6c5)c5cccc6ccccc56)c4)c4cccc5ccccc45)c3)cc2)cc1]

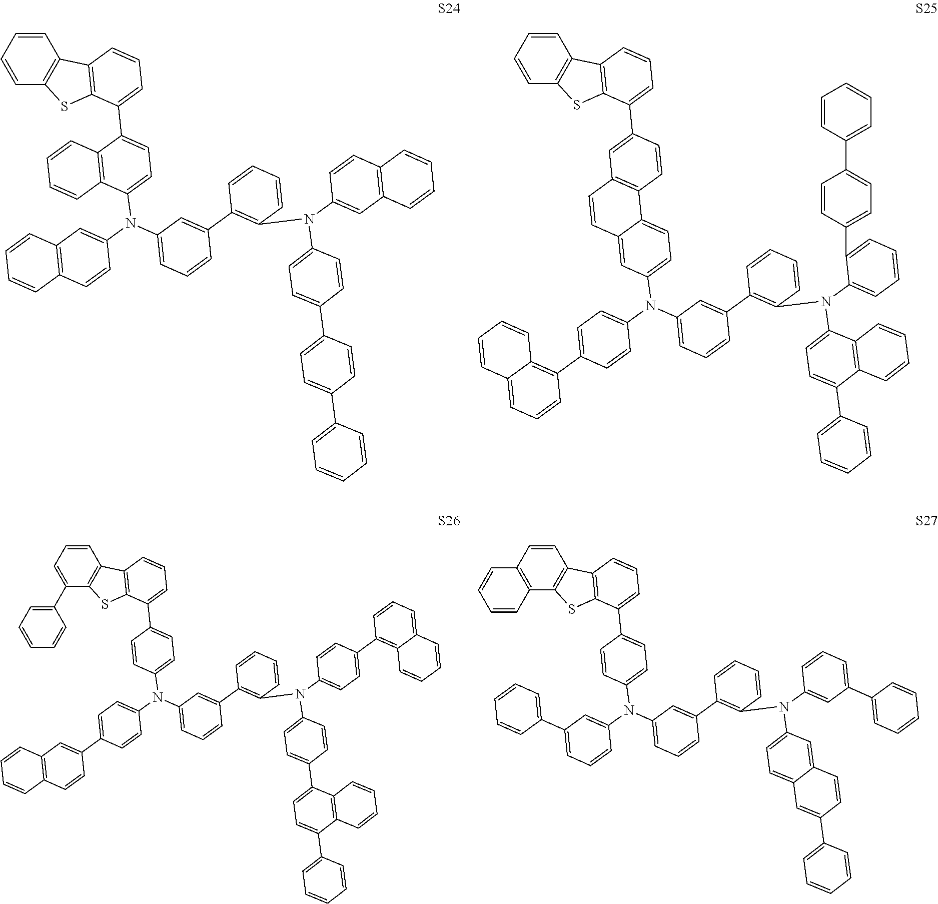

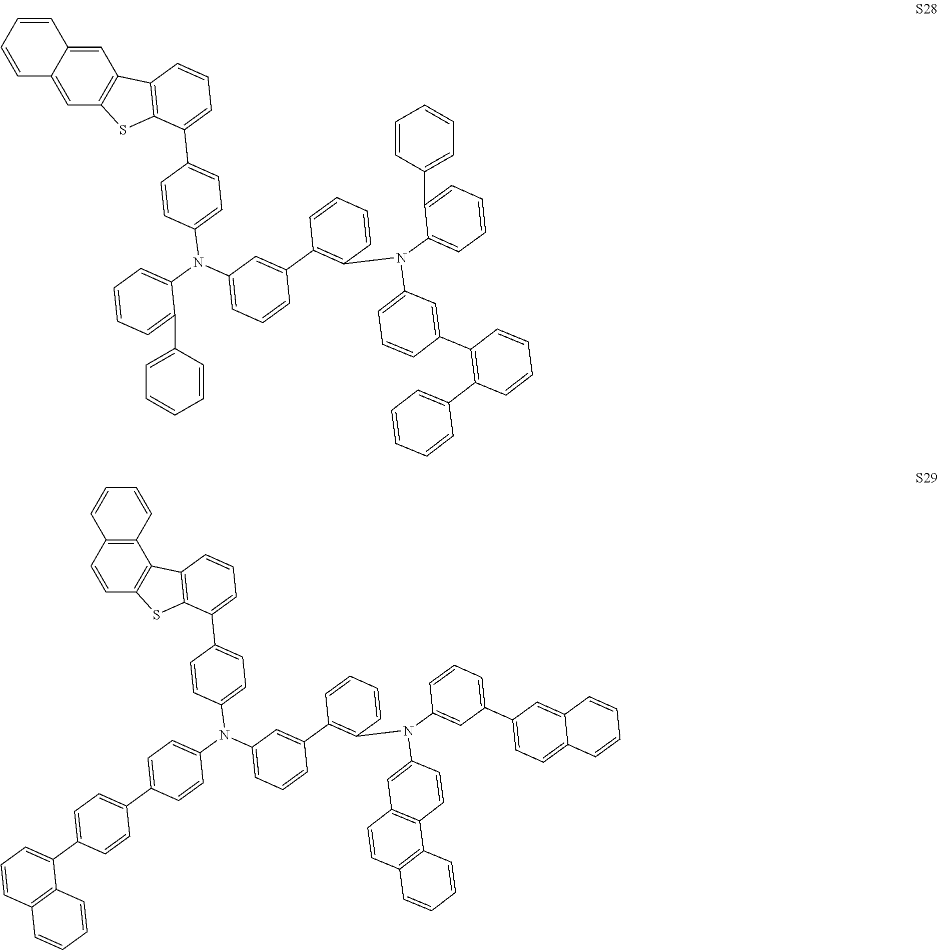 [SMILES: c1ccc(-c2ccccc2-c2cccc(N(c3ccccc3-c3ccccc3)c3ccccc3-c3cccc(N(c4ccc(-c5cccc6c5sc5cc7ccccc7cc56)cc4)c4ccccc4-c4ccccc4)c3)c2)cc1 ; c1cc(-c2ccccc2N(c2cccc(-c3ccc4ccccc4c3)c2)c2ccc3c(ccc4ccccc43)c2)cc(N(c2ccc(-c3ccc(-c4cccc5ccccc45)cc3)cc2)c2ccc(-c3cccc4c3sc3ccc5ccccc5c34)cc2)c1]

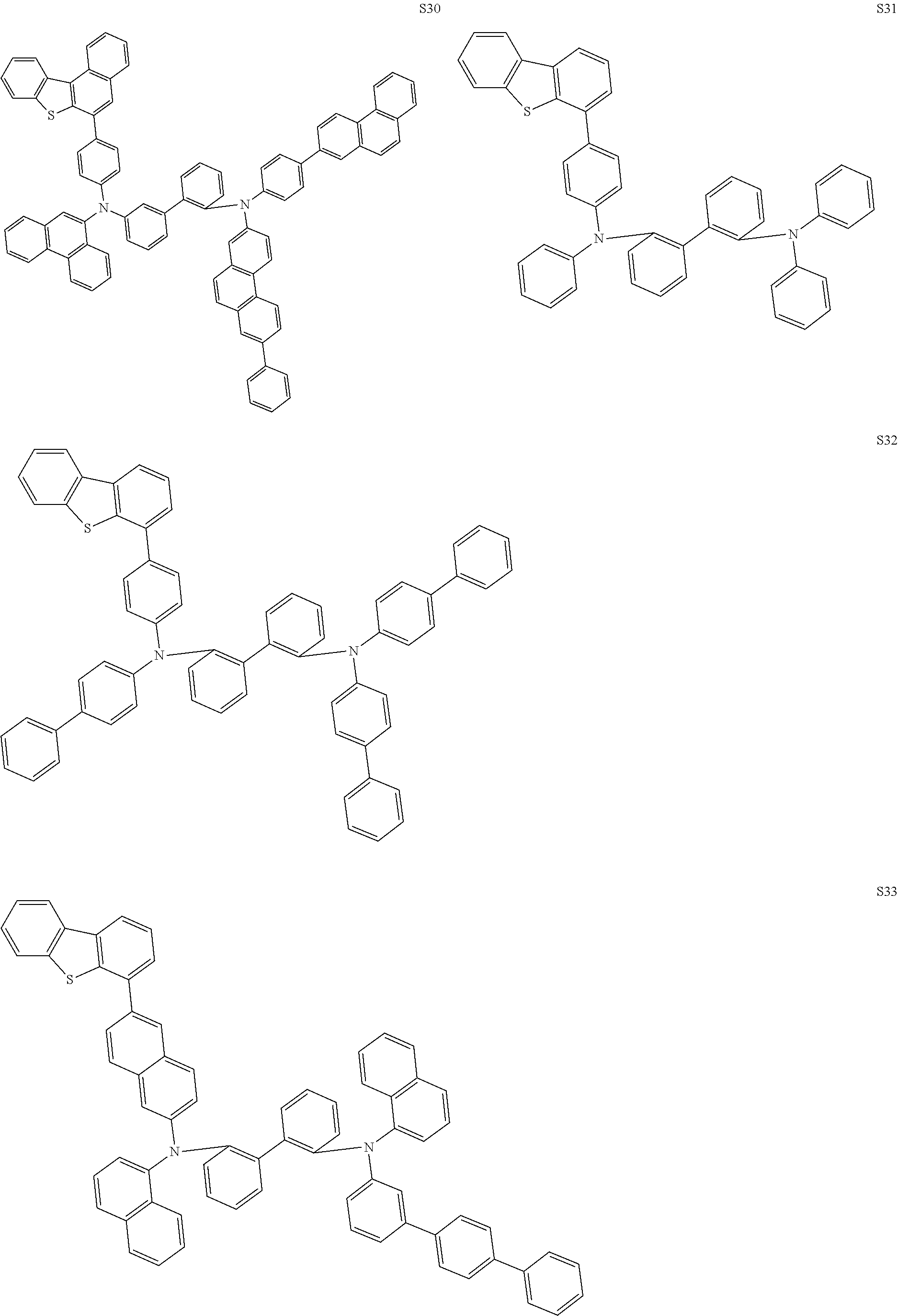 [SMILES: c1ccc(-c2ccc3c(ccc4cc(N(c5ccc(-c6ccc7c(ccc8ccccc87)c6)cc5)c5ccccc5-c5cccc(N(c6ccc(-c7cc8ccccc8c8c7sc7ccccc78)cc6)c6cc7ccccc7c7ccccc67)c5)ccc43)c2)cc1 ; c1ccc(N(c2ccccc2)c2ccccc2-c2ccccc2N(c2ccccc2)c2ccc(-c3cccc4c3sc3ccccc34)cc2)cc1 ; c1ccc(-c2ccc(N(c3ccc(-c4ccccc4)cc3)c3ccccc3-c3ccccc3N(c3ccc(-c4ccccc4)cc3)c3ccc(-c4cccc5c4sc4ccccc45)cc3)cc2)cc1 ; c1ccc(-c2ccc(-c3cccc(N(c4ccccc4-c4ccccc4N(c4ccc5cc(-c6cccc7c6sc6ccccc67)ccc5c4)c4cccc5ccccc45)c4cccc5ccccc45)c3)cc2)cc1]

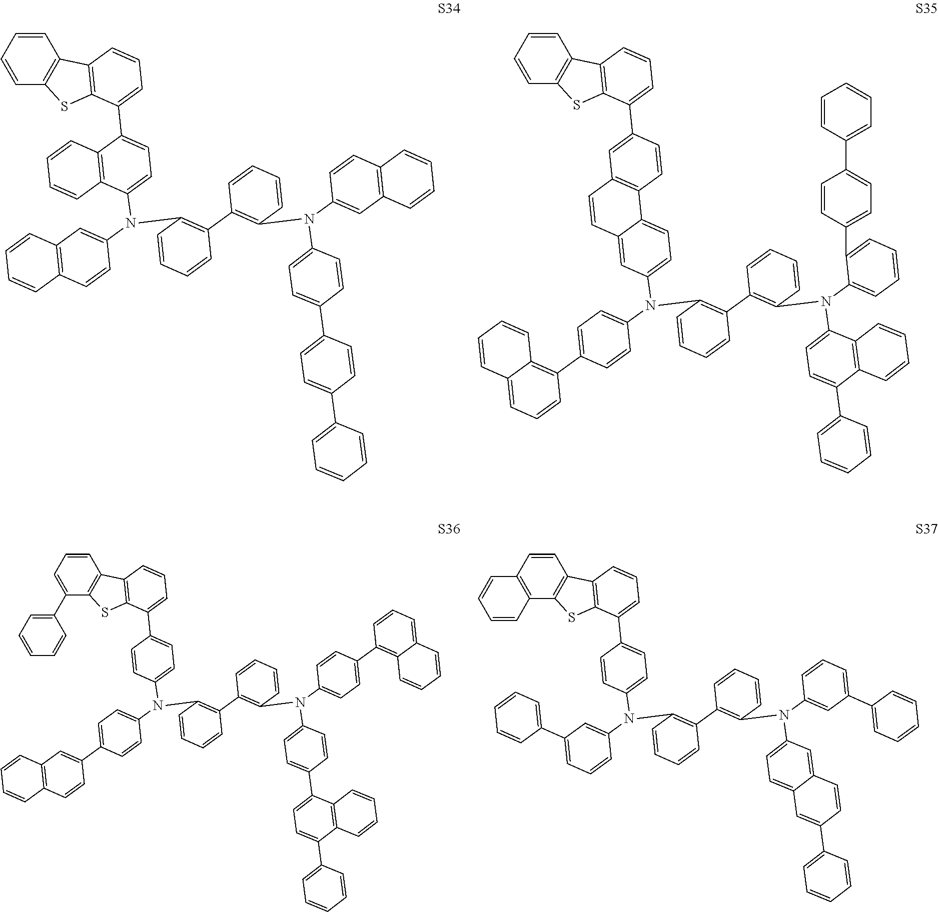

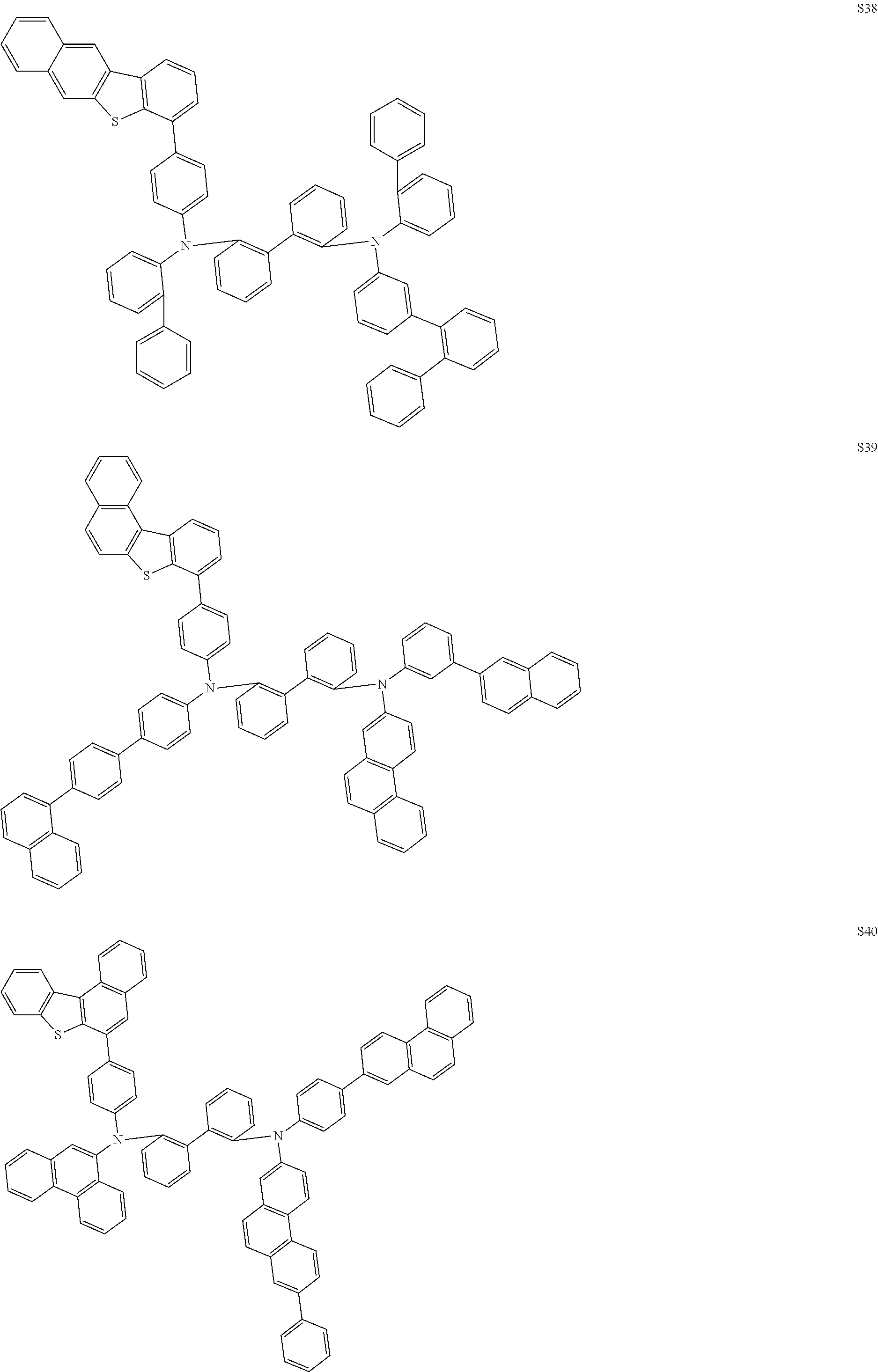 [SMILES: c1ccc(-c2ccccc2-c2cccc(N(c3ccccc3-c3ccccc3)c3ccccc3-c3ccccc3N(c3ccc(-c4cccc5c4sc4cc6ccccc6cc45)cc3)c3ccccc3-c3ccccc3)c2)cc1 ; c1cc(-c2ccc3ccccc3c2)cc(N(c2ccc3c(ccc4ccccc43)c2)c2ccccc2-c2ccccc2N(c2ccc(-c3ccc(-c4cccc5ccccc45)cc3)cc2)c2ccc(-c3cccc4c3sc3ccc5ccccc5c34)cc2)c1 ; c1ccc(-c2ccc3c(ccc4cc(N(c5ccc(-c6ccc7c(ccc8ccccc87)c6)cc5)c5ccccc5-c5ccccc5N(c5ccc(-c6cc7ccccc7c7c6sc6ccccc67)cc5)c5cc6ccccc6c6ccccc56)ccc43)c2)cc1]

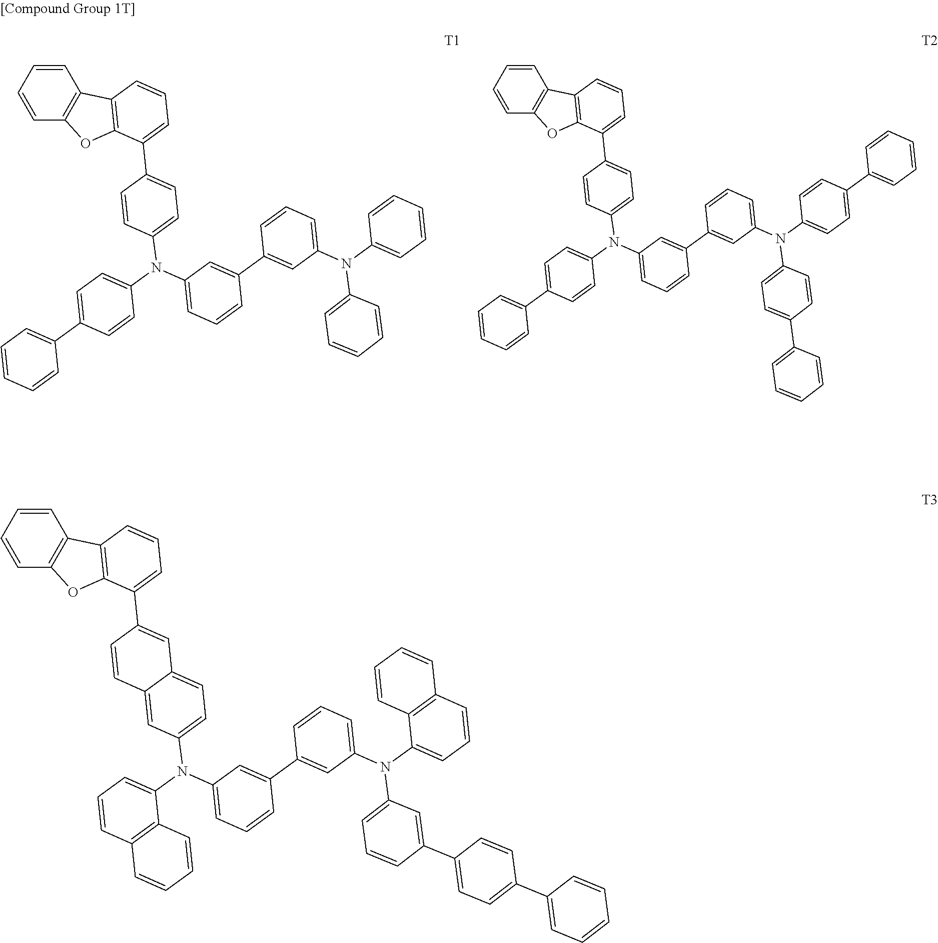 [SMILES: c1ccc(-c2ccc(N(c3ccc(-c4cccc5c4oc4ccccc45)cc3)c3cccc(-c4cccc(N(c5ccccc5)c5ccccc5)c4)c3)cc2)cc1 ; c1ccc(-c2ccc(N(c3ccc(-c4ccccc4)cc3)c3cccc(-c4cccc(N(c5ccc(-c6ccccc6)cc5)c5ccc(-c6cccc7c6oc6ccccc67)cc5)c4)c3)cc2)cc1 ; c1ccc(-c2ccc(-c3cccc(N(c4cccc(-c5cccc(N(c6ccc7cc(-c8cccc9c8oc8ccccc89)ccc7c6)c6cccc7ccccc67)c5)c4)c4cccc5ccccc45)c3)cc2)cc1]

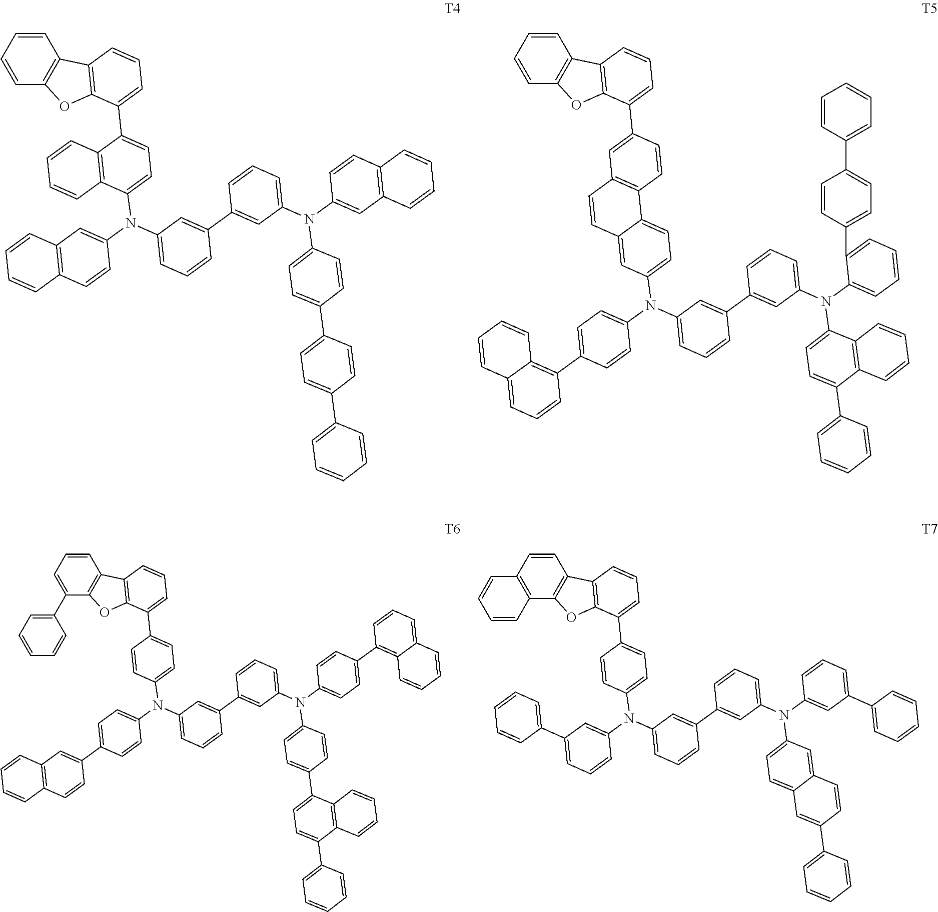

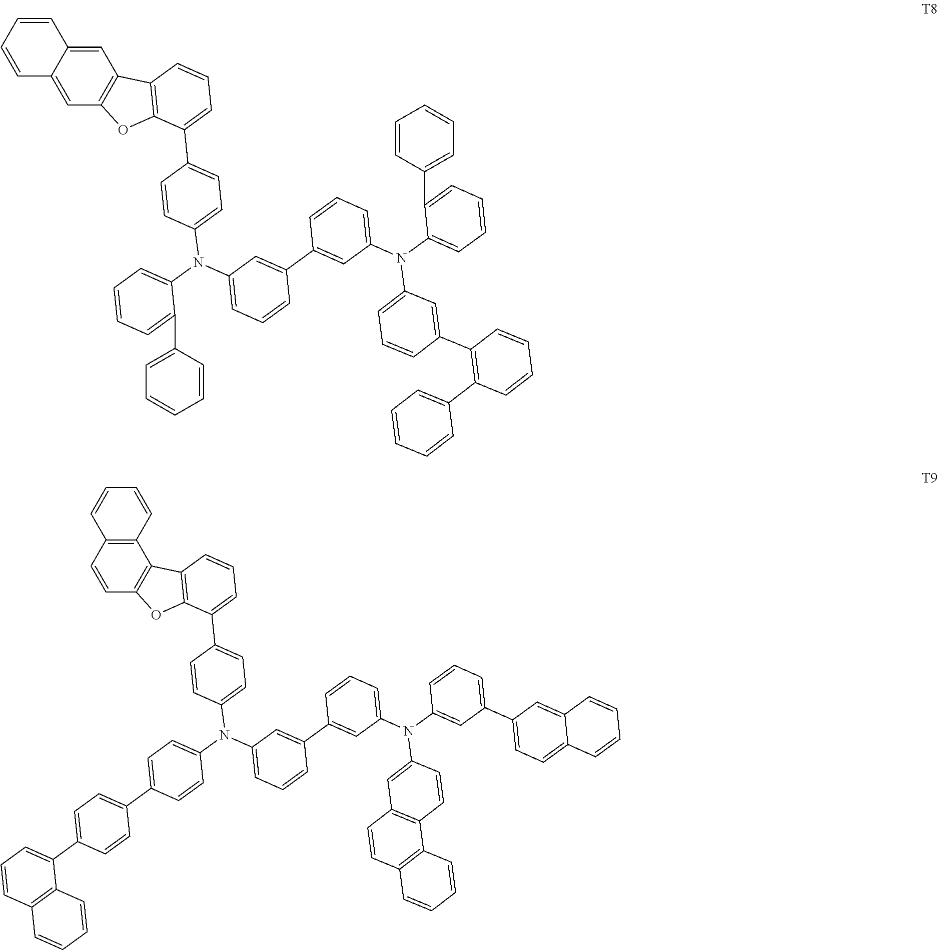 [SMILES: c1ccc(-c2ccccc2-c2cccc(N(c3cccc(-c4cccc(N(c5ccc(-c6cccc7c6oc6cc8ccccc8cc67)cc5)c5ccccc5-c5ccccc5)c4)c3)c3ccccc3-c3ccccc3)c2)cc1 ; c1cc(-c2cccc(N(c3cccc(-c4ccc5ccccc5c4)c3)c3ccc4c(ccc5ccccc54)c3)c2)cc(N(c2ccc(-c3ccc(-c4cccc5ccccc45)cc3)cc2)c2ccc(-c3cccc4c3oc3ccc5ccccc5c34)cc2)c1]

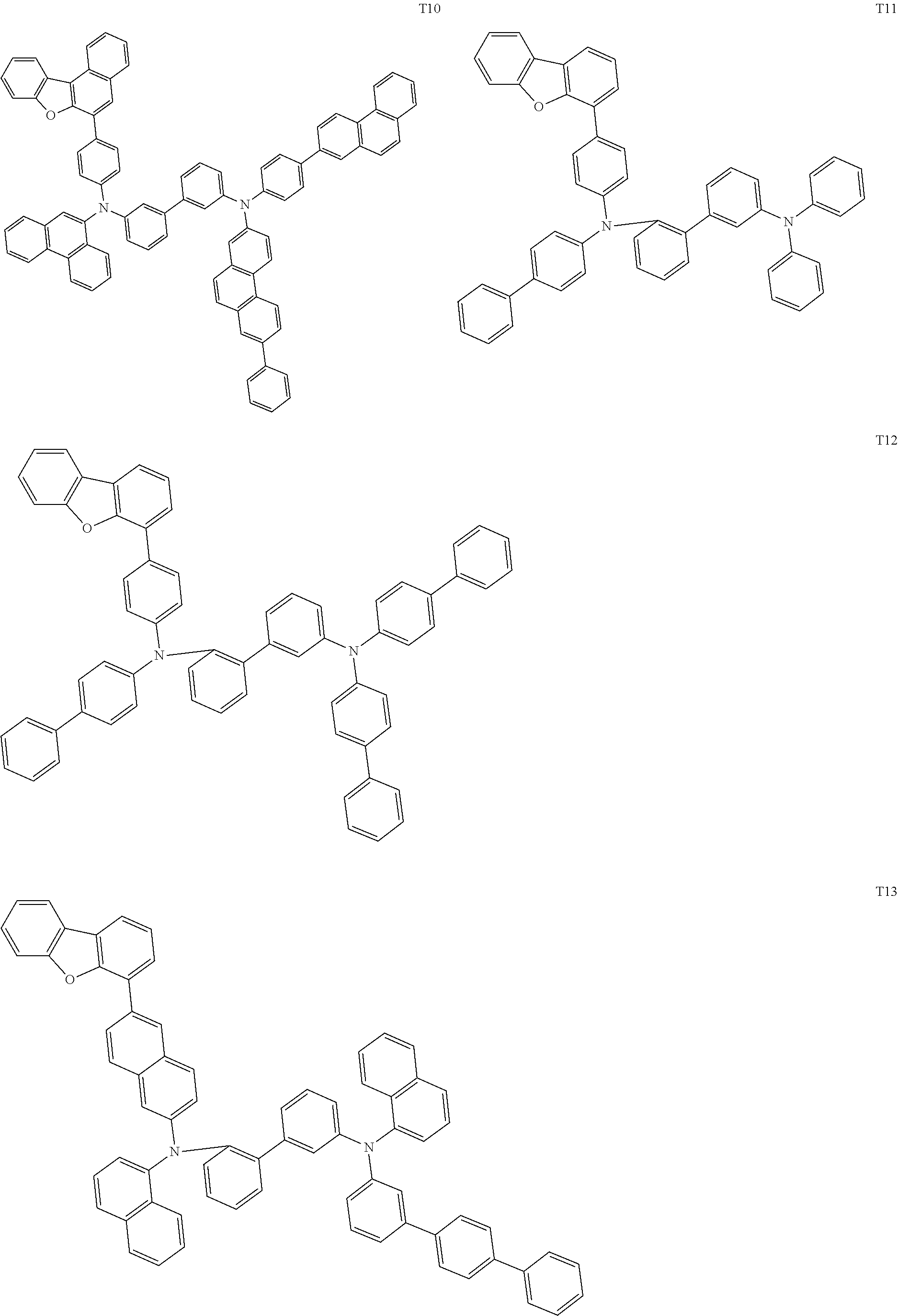 [SMILES: c1ccc(-c2ccc3c(ccc4cc(N(c5ccc(-c6ccc7c(ccc8ccccc87)c6)cc5)c5cccc(-c6cccc(N(c7ccc(-c8cc9ccccc9c9c8oc8ccccc89)cc7)c7cc8ccccc8c8ccccc78)c6)c5)ccc43)c2)cc1 ; c1ccc(-c2ccc(N(c3ccc(-c4cccc5c4oc4ccccc45)cc3)c3ccccc3-c3cccc(N(c4ccccc4)c4ccccc4)c3)cc2)cc1 ; c1ccc(-c2ccc(N(c3ccc(-c4ccccc4)cc3)c3cccc(-c4ccccc4N(c4ccc(-c5ccccc5)cc4)c4ccc(-c5cccc6c5oc5ccccc56)cc4)c3)cc2)cc1 ; c1ccc(-c2ccc(-c3cccc(N(c4cccc(-c5ccccc5N(c5ccc6cc(-c7cccc8c7oc7ccccc78)ccc6c5)c5cccc6ccccc56)c4)c4cccc5ccccc45)c3)cc2)cc1]

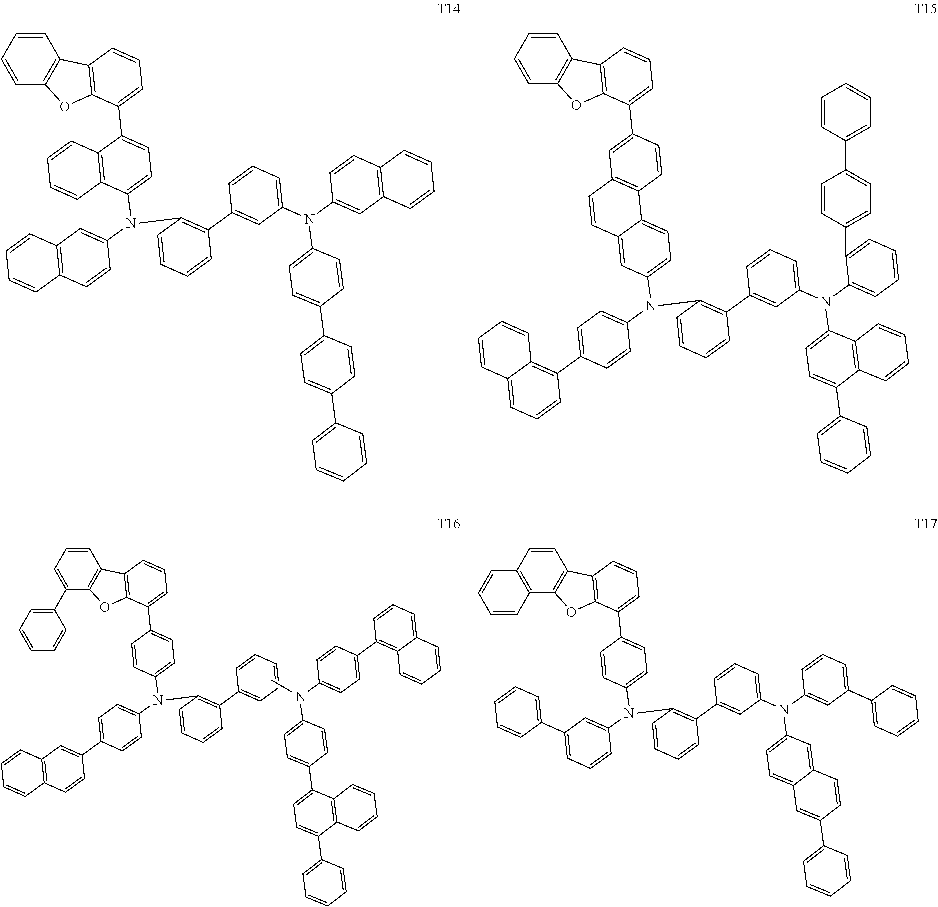

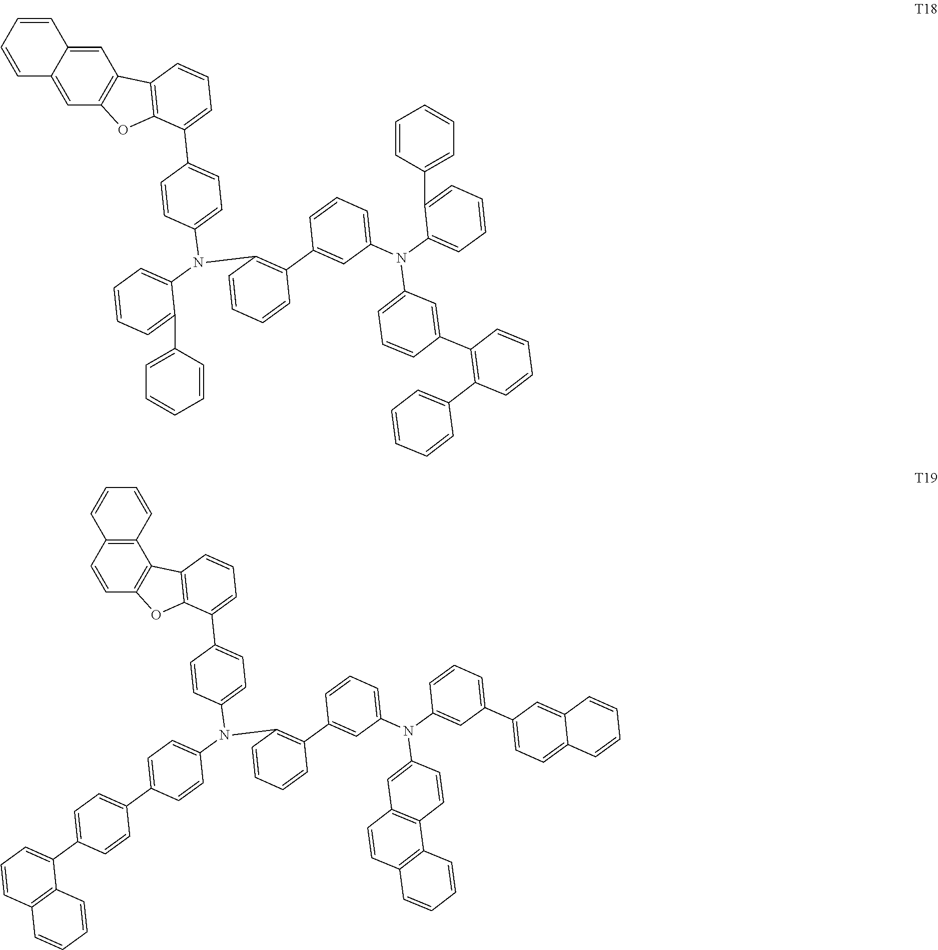 [SMILES: c1ccc(-c2ccccc2-c2cccc(N(c3cccc(-c4ccccc4N(c4ccc(-c5cccc6c5oc5cc7ccccc7cc56)cc4)c4ccccc4-c4ccccc4)c3)c3ccccc3-c3ccccc3)c2)cc1 ; c1cc(-c2ccc3ccccc3c2)cc(N(c2cccc(-c3ccccc3N(c3ccc(-c4ccc(-c5cccc6ccccc56)cc4)cc3)c3ccc(-c4cccc5c4oc4ccc6ccccc6c45)cc3)c2)c2ccc3c(ccc4ccccc43)c2)c1]

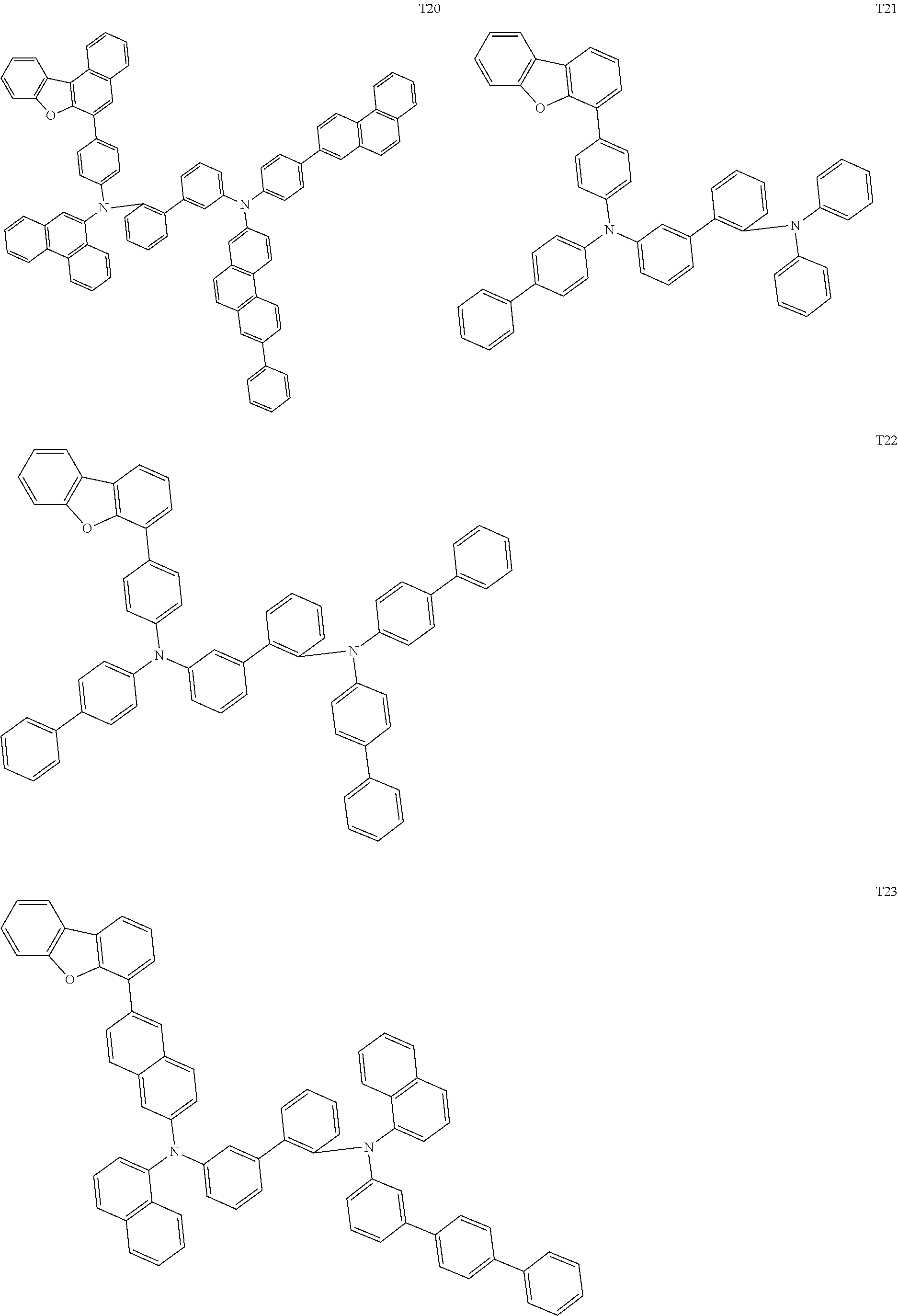 [SMILES: c1ccc(-c2ccc3c(ccc4cc(N(c5ccc(-c6ccc7c(ccc8ccccc87)c6)cc5)c5cccc(-c6ccccc6N(c6ccc(-c7cc8ccccc8c8c7oc7ccccc78)cc6)c6cc7ccccc7c7ccccc67)c5)ccc43)c2)cc1 ; c1ccc(-c2ccc(N(c3ccc(-c4cccc5c4oc4ccccc45)cc3)c3cccc(-c4ccccc4N(c4ccccc4)c4ccccc4)c3)cc2)cc1 ; c1ccc(-c2ccc(N(c3ccc(-c4cccc5c4oc4ccccc45)cc3)c3cccc(-c4ccccc4N(c4ccc(-c5ccccc5)cc4)c4ccc(-c5ccccc5)cc4)c3)cc2)cc1 ; c1ccc(-c2ccc(-c3cccc(N(c4ccccc4-c4cccc(N(c5ccc6cc(-c7cccc8c7oc7ccccc78)ccc6c5)c5cccc6ccccc56)c4)c4cccc5ccccc45)c3)cc2)cc1]

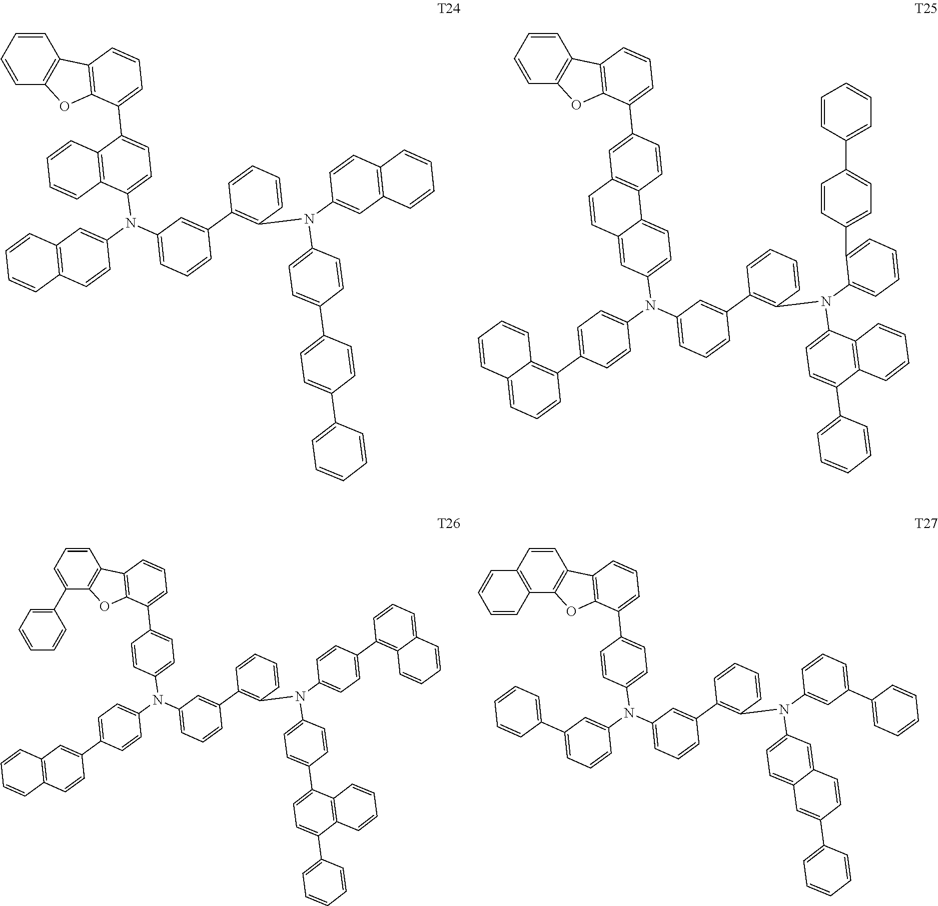

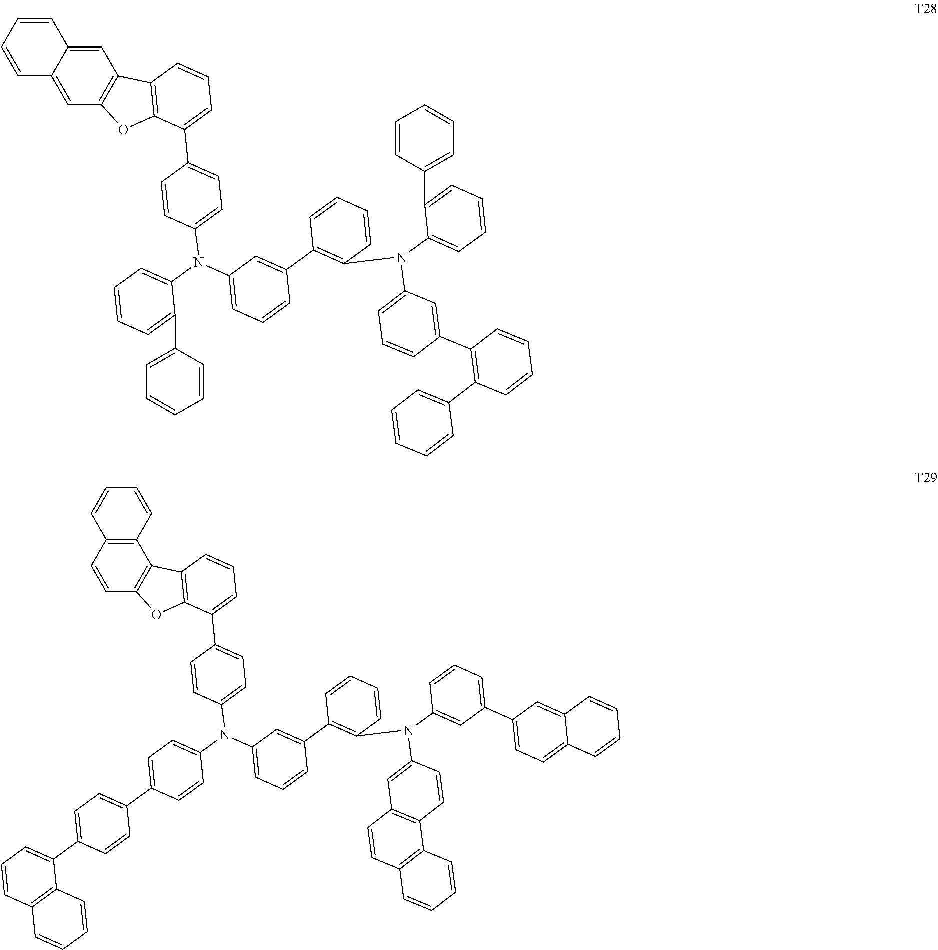 [SMILES: c1ccc(-c2ccccc2-c2cccc(N(c3ccccc3-c3ccccc3)c3ccccc3-c3cccc(N(c4ccc(-c5cccc6c5oc5cc7ccccc7cc56)cc4)c4ccccc4-c4ccccc4)c3)c2)cc1 ; c1cc(-c2ccccc2N(c2cccc(-c3ccc4ccccc4c3)c2)c2ccc3c(ccc4ccccc43)c2)cc(N(c2ccc(-c3ccc(-c4cccc5ccccc45)cc3)cc2)c2ccc(-c3cccc4c3oc3ccc5ccccc5c34)cc2)c1]

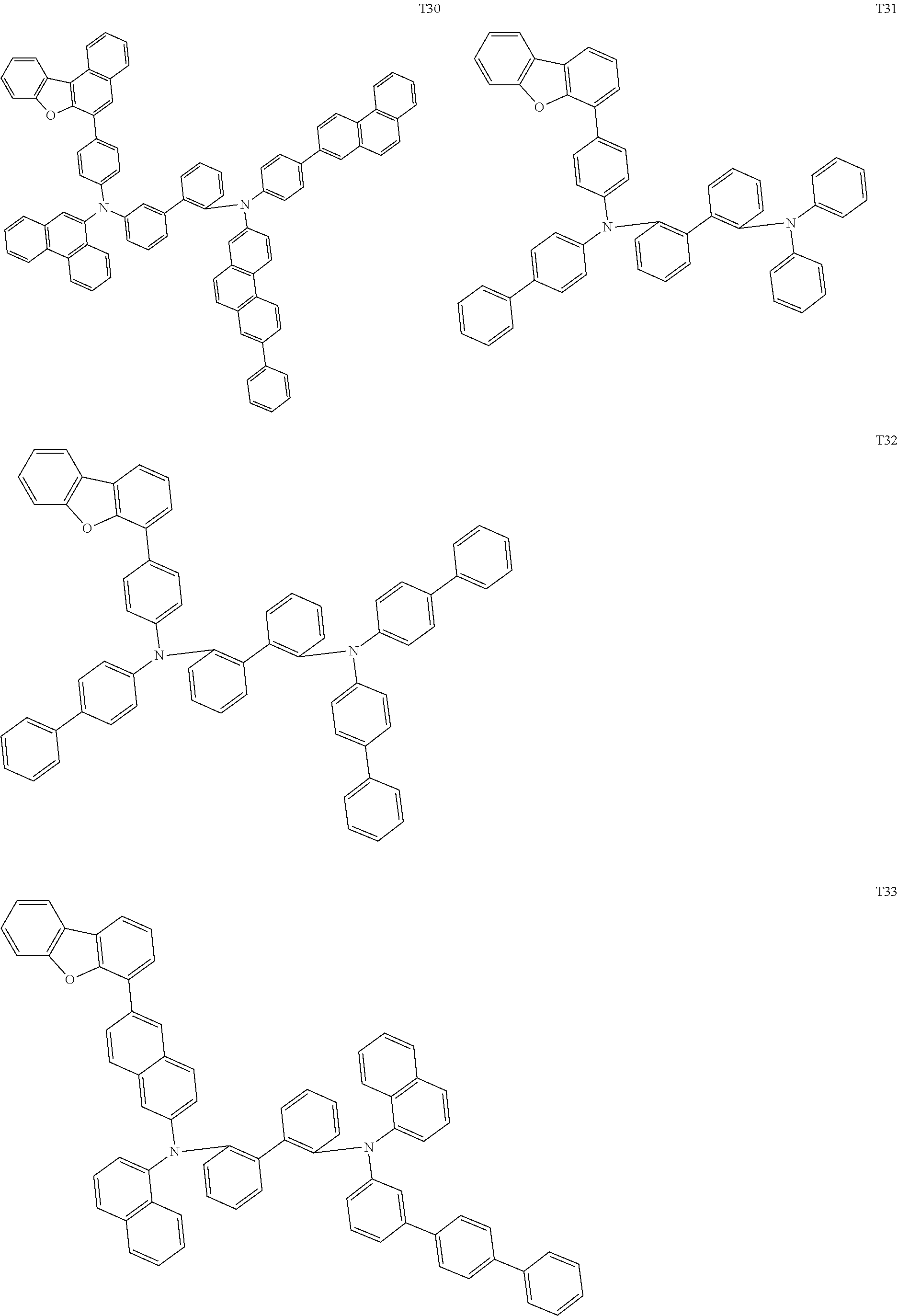 [SMILES: c1ccc(-c2ccc3c(ccc4cc(N(c5ccc(-c6ccc7c(ccc8ccccc87)c6)cc5)c5ccccc5-c5cccc(N(c6ccc(-c7cc8ccccc8c8c7oc7ccccc78)cc6)c6cc7ccccc7c7ccccc67)c5)ccc43)c2)cc1 ; c1ccc(-c2ccc(N(c3ccc(-c4cccc5c4oc4ccccc45)cc3)c3ccccc3-c3ccccc3N(c3ccccc3)c3ccccc3)cc2)cc1 ; c1ccc(-c2ccc(N(c3ccc(-c4ccccc4)cc3)c3ccccc3-c3ccccc3N(c3ccc(-c4ccccc4)cc3)c3ccc(-c4cccc5c4oc4ccccc45)cc3)cc2)cc1 ; c1ccc(-c2ccc(-c3cccc(N(c4ccccc4-c4ccccc4N(c4ccc5cc(-c6cccc7c6oc6ccccc67)ccc5c4)c4cccc5ccccc45)c4cccc5ccccc45)c3)cc2)cc1]

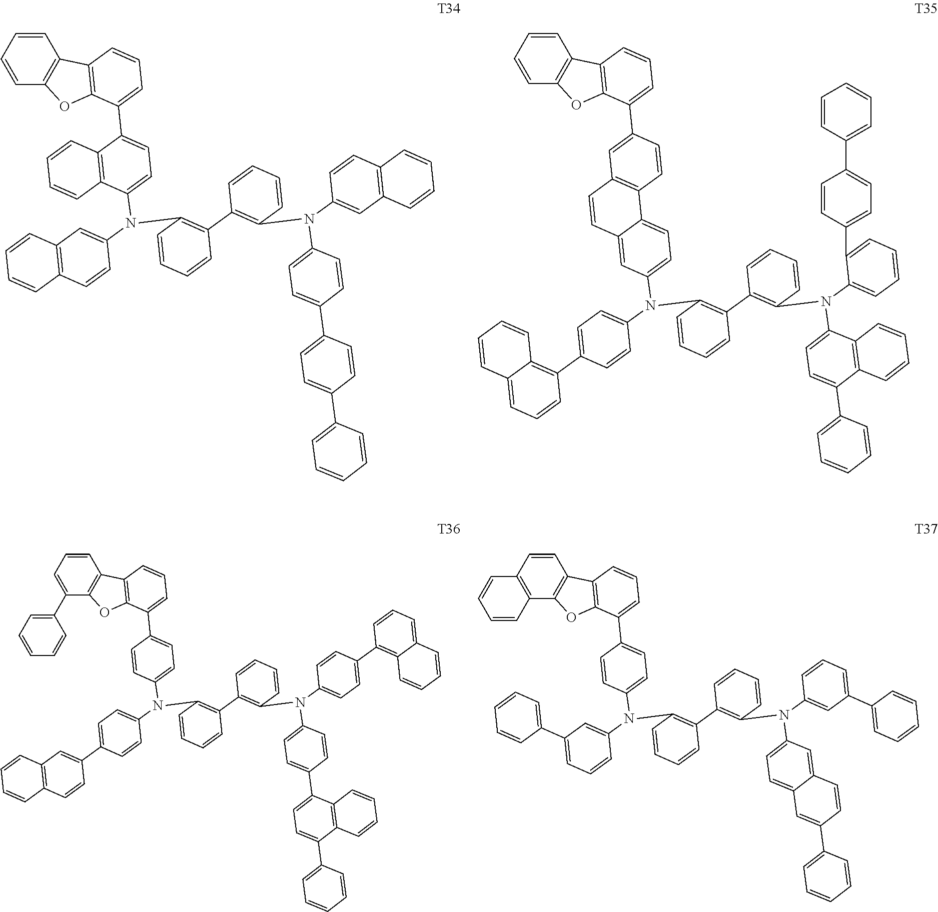

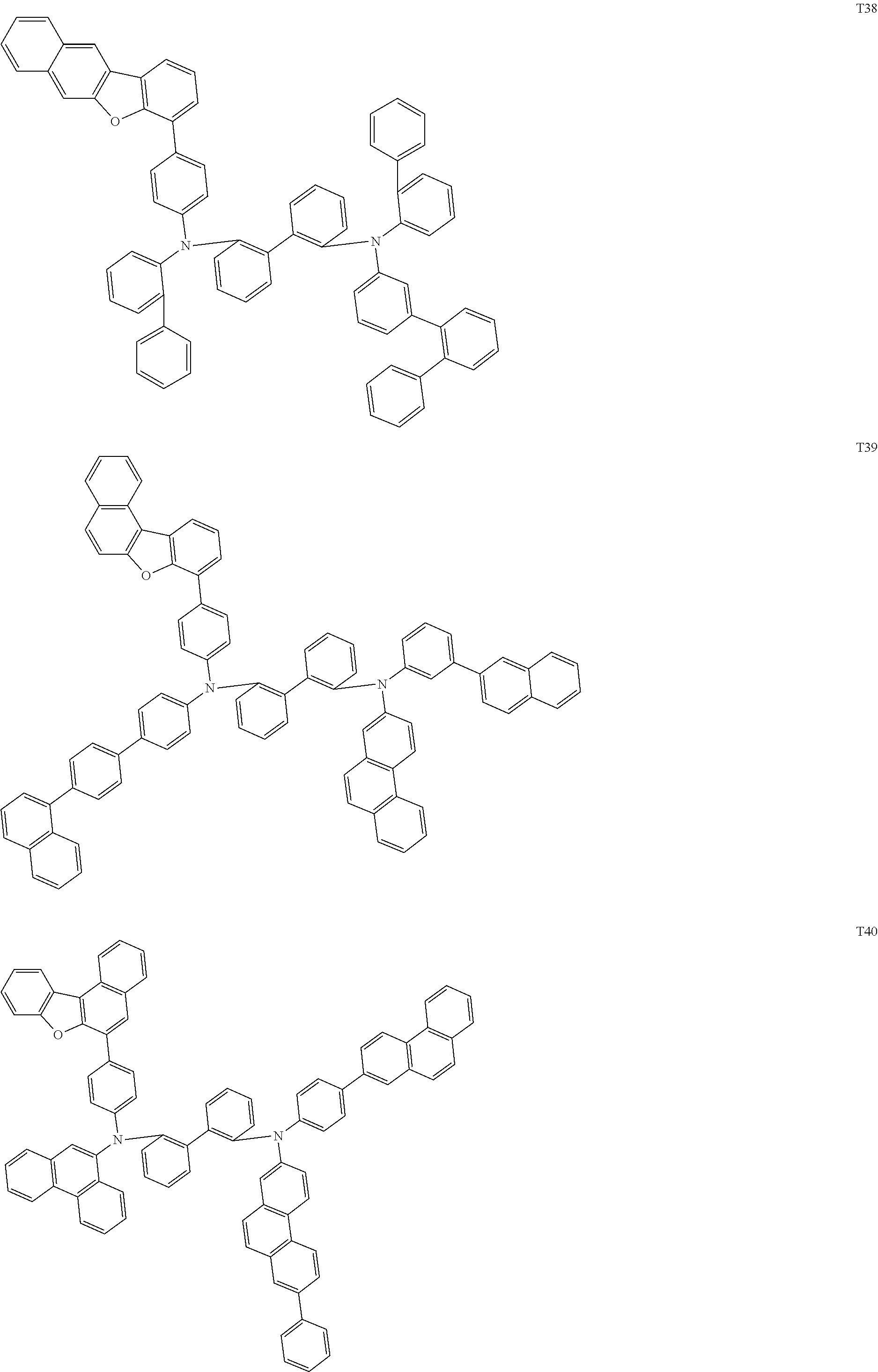 [SMILES: c1ccc(-c2ccccc2-c2cccc(N(c3ccccc3-c3ccccc3)c3ccccc3-c3ccccc3N(c3ccc(-c4cccc5c4oc4cc6ccccc6cc45)cc3)c3ccccc3-c3ccccc3)c2)cc1 ; c1cc(-c2ccc3ccccc3c2)cc(N(c2ccc3c(ccc4ccccc43)c2)c2ccccc2-c2ccccc2N(c2ccc(-c3ccc(-c4cccc5ccccc45)cc3)cc2)c2ccc(-c3cccc4c3oc3ccc5ccccc5c34)cc2)c1 ; c1ccc(-c2ccc3c(ccc4cc(N(c5ccc(-c6ccc7c(ccc8ccccc87)c6)cc5)c5ccccc5-c5ccccc5N(c5ccc(-c6cc7ccccc7c7c6oc6ccccc67)cc5)c5cc6ccccc6c6ccccc56)ccc43)c2)cc1]

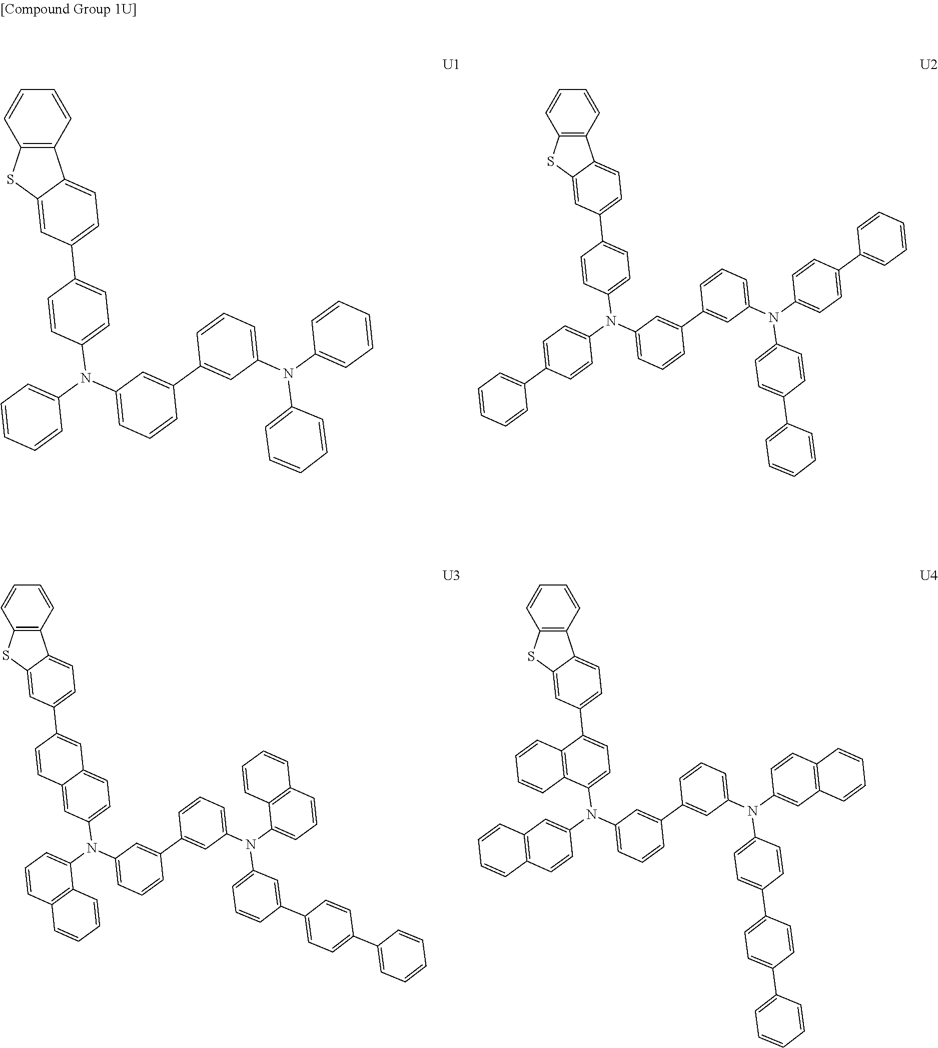 [SMILES: c1ccc(N(c2ccccc2)c2cccc(-c3cccc(N(c4ccccc4)c4ccc(-c5ccc6c(c5)sc5ccccc56)cc4)c3)c2)cc1 ; c1ccc(-c2ccc(N(c3ccc(-c4ccccc4)cc3)c3cccc(-c4cccc(N(c5ccc(-c6ccccc6)cc5)c5ccc(-c6ccc7c(c6)sc6ccccc67)cc5)c4)c3)cc2)cc1 ; c1ccc(-c2ccc(-c3cccc(N(c4cccc(-c5cccc(N(c6ccc7cc(-c8ccc9c(c8)sc8ccccc89)ccc7c6)c6cccc7ccccc67)c5)c4)c4cccc5ccccc45)c3)cc2)cc1 ; c1ccc(-c2ccc(-c3ccc(N(c4cccc(-c5cccc(N(c6ccc7ccccc7c6)c6ccc(-c7ccc8c(c7)sc7ccccc78)c7ccccc67)c5)c4)c4ccc5ccccc5c4)cc3)cc2)cc1]

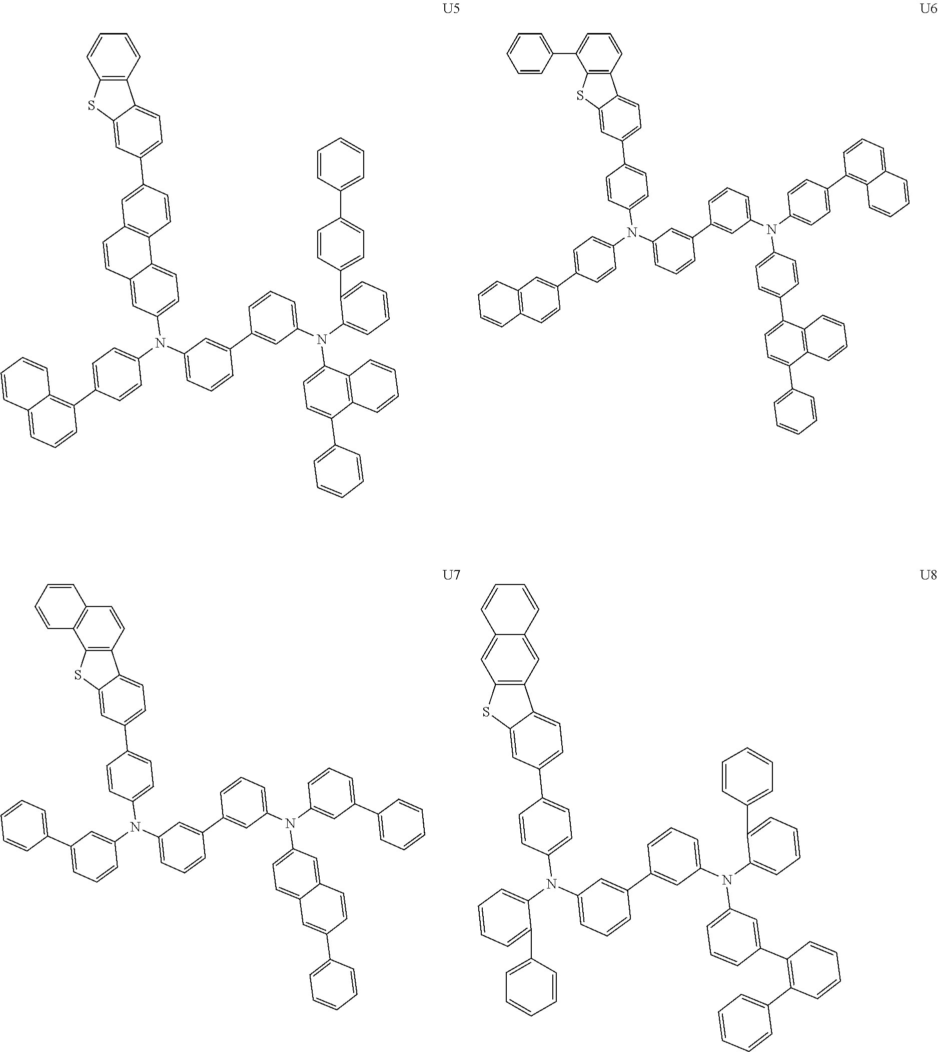

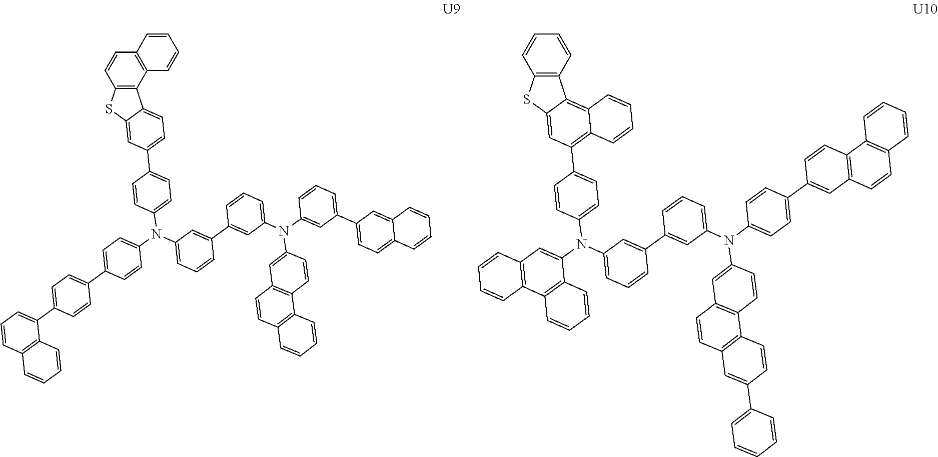 [SMILES: c1cc(-c2cccc(N(c3cccc(-c4ccc5ccccc5c4)c3)c3ccc4c(ccc5ccccc54)c3)c2)cc(N(c2ccc(-c3ccc(-c4cccc5ccccc45)cc3)cc2)c2ccc(-c3ccc4c(c3)sc3ccc5ccccc5c34)cc2)c1 ; c1ccc(-c2ccc3c(ccc4cc(N(c5ccc(-c6ccc7c(ccc8ccccc87)c6)cc5)c5cccc(-c6cccc(N(c7ccc(-c8cc9sc%10ccccc%10c9c9ccccc89)cc7)c7cc8ccccc8c8ccccc78)c6)c5)ccc43)c2)cc1]

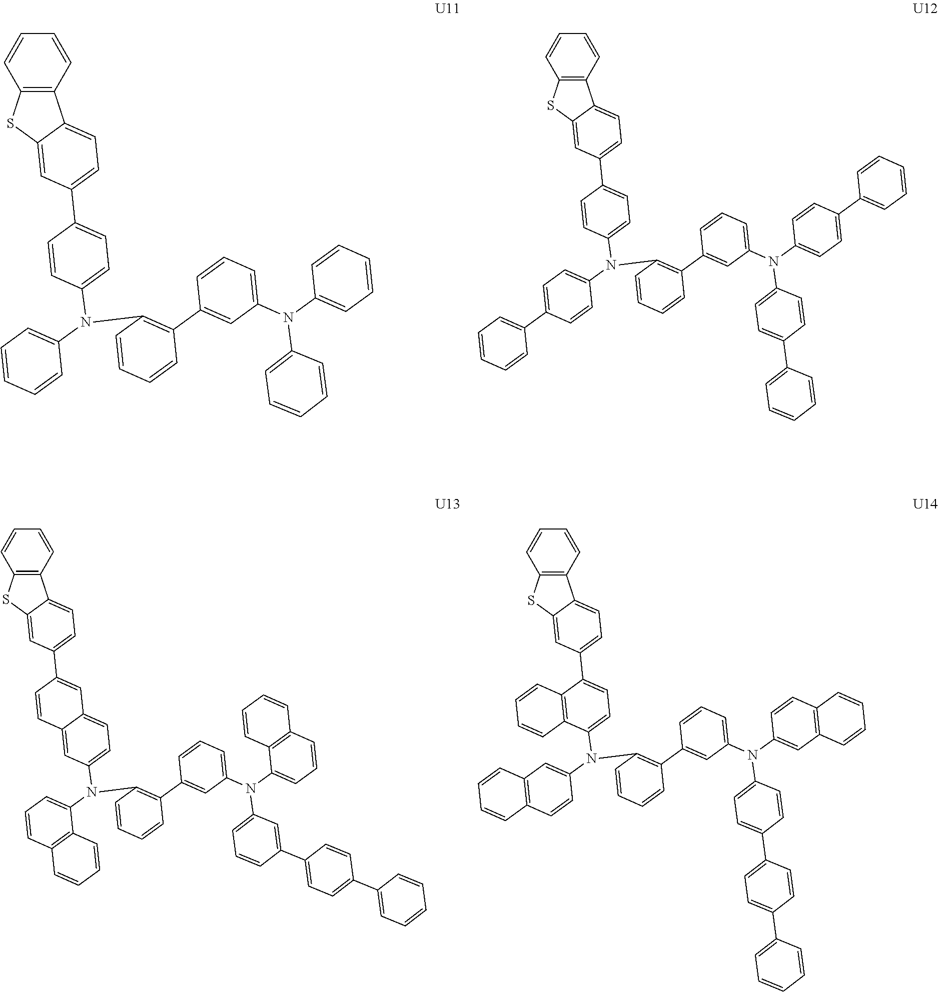 [SMILES: c1ccc(N(c2ccccc2)c2cccc(-c3ccccc3N(c3ccccc3)c3ccc(-c4ccc5c(c4)sc4ccccc45)cc3)c2)cc1 ; c1ccc(-c2ccc(N(c3ccc(-c4ccccc4)cc3)c3cccc(-c4ccccc4N(c4ccc(-c5ccccc5)cc4)c4ccc(-c5ccc6c(c5)sc5ccccc56)cc4)c3)cc2)cc1 ; c1ccc(-c2ccc(-c3cccc(N(c4cccc(-c5ccccc5N(c5ccc6cc(-c7ccc8c(c7)sc7ccccc78)ccc6c5)c5cccc6ccccc56)c4)c4cccc5ccccc45)c3)cc2)cc1 ; c1ccc(-c2ccc(-c3ccc(N(c4cccc(-c5ccccc5N(c5ccc6ccccc6c5)c5ccc(-c6ccc7c(c6)sc6ccccc67)c6ccccc56)c4)c4ccc5ccccc5c4)cc3)cc2)cc1]

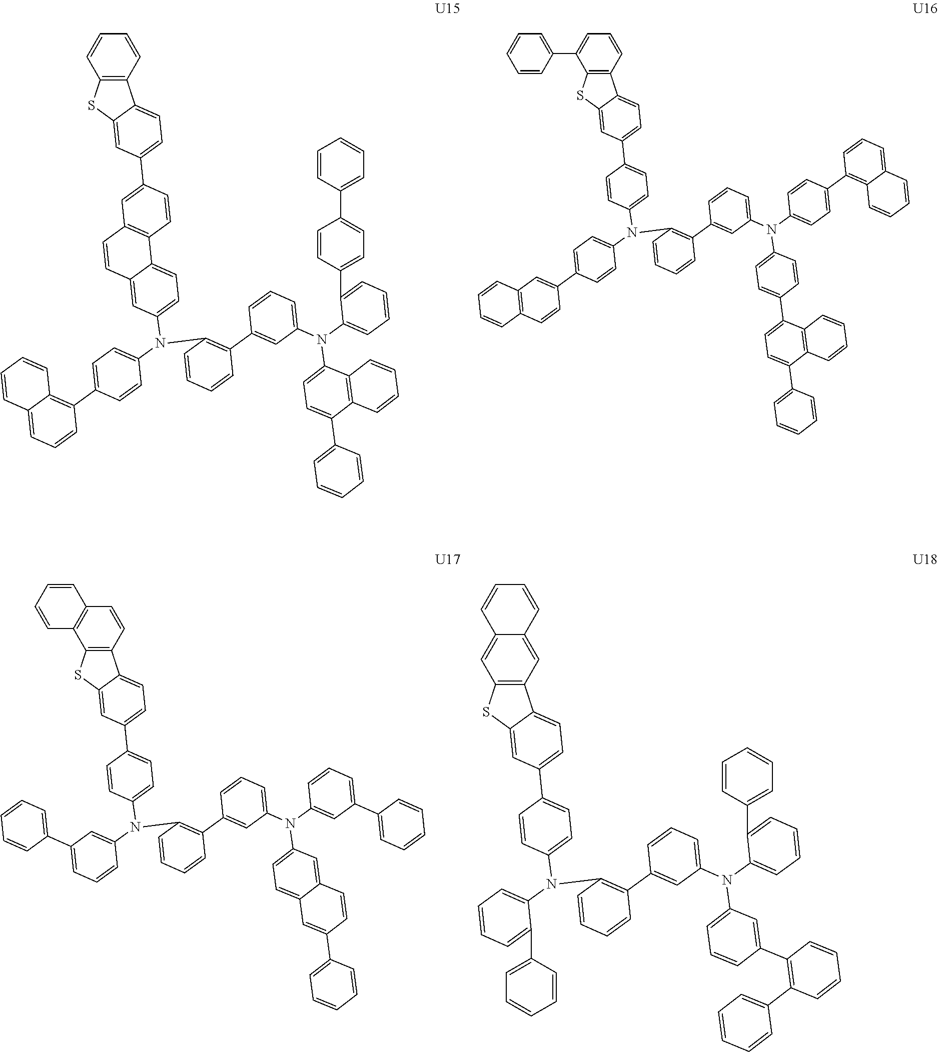

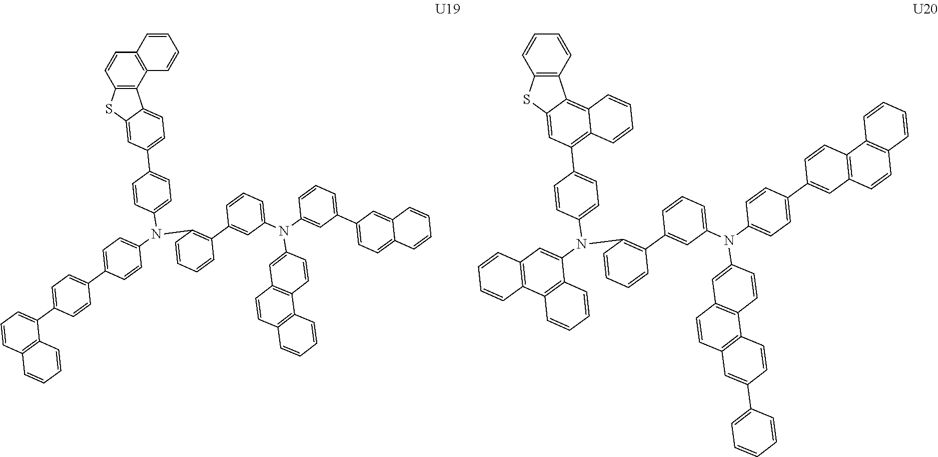 [SMILES: c1cc(-c2ccc3ccccc3c2)cc(N(c2cccc(-c3ccccc3N(c3ccc(-c4ccc(-c5cccc6ccccc56)cc4)cc3)c3ccc(-c4ccc5c(c4)sc4ccc6ccccc6c45)cc3)c2)c2ccc3c(ccc4ccccc43)c2)c1 ; c1ccc(-c2ccc3c(ccc4cc(N(c5ccc(-c6ccc7c(ccc8ccccc87)c6)cc5)c5cccc(-c6ccccc6N(c6ccc(-c7cc8sc9ccccc9c8c8ccccc78)cc6)c6cc7ccccc7c7ccccc67)c5)ccc43)c2)cc1]

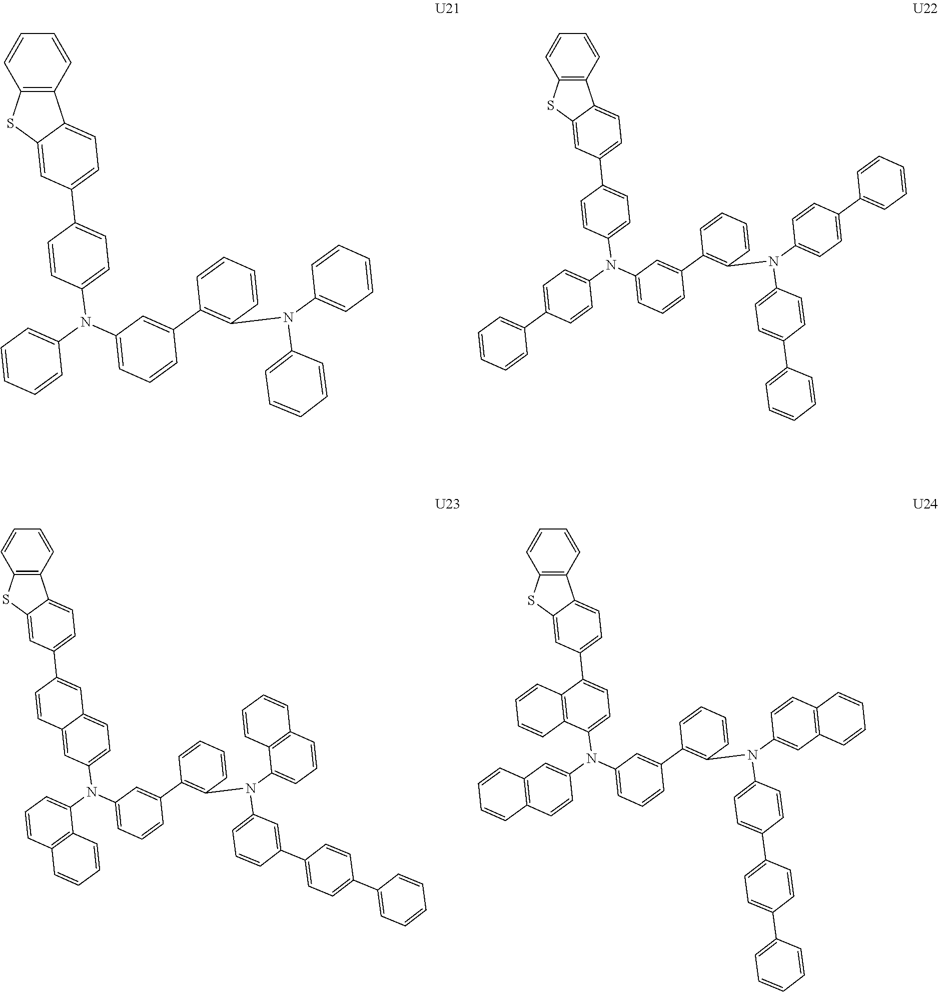 [SMILES: c1ccc(N(c2ccc(-c3ccc4c(c3)sc3ccccc34)cc2)c2cccc(-c3ccccc3N(c3ccccc3)c3ccccc3)c2)cc1 ; c1ccc(-c2ccc(N(c3ccc(-c4ccc5c(c4)sc4ccccc45)cc3)c3cccc(-c4ccccc4N(c4ccc(-c5ccccc5)cc4)c4ccc(-c5ccccc5)cc4)c3)cc2)cc1 ; c1ccc(-c2ccc(-c3cccc(N(c4ccccc4-c4cccc(N(c5ccc6cc(-c7ccc8c(c7)sc7ccccc78)ccc6c5)c5cccc6ccccc56)c4)c4cccc5ccccc45)c3)cc2)cc1 ; c1ccc(-c2ccc(-c3ccc(N(c4ccc5ccccc5c4)c4ccccc4-c4cccc(N(c5ccc6ccccc6c5)c5ccc(-c6ccc7c(c6)sc6ccccc67)c6ccccc56)c4)cc3)cc2)cc1]

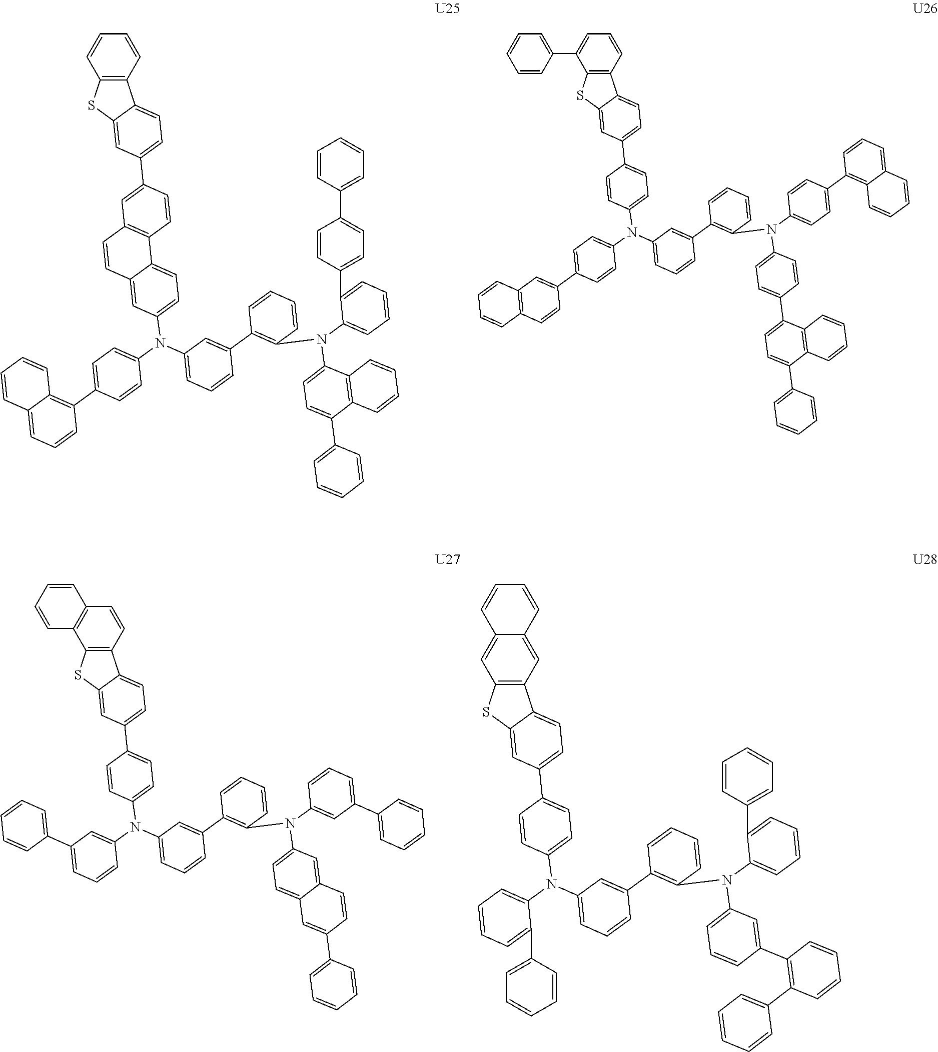

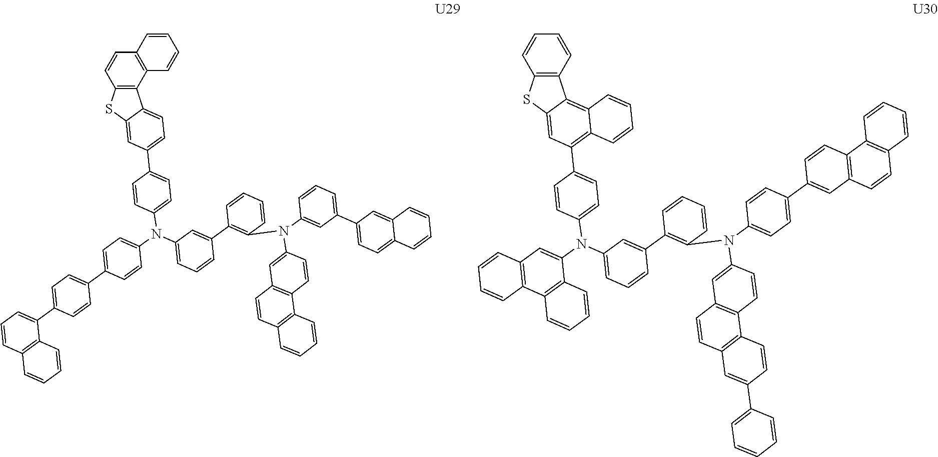 [SMILES: c1cc(-c2ccccc2N(c2cccc(-c3ccc4ccccc4c3)c2)c2ccc3c(ccc4ccccc43)c2)cc(N(c2ccc(-c3ccc(-c4cccc5ccccc45)cc3)cc2)c2ccc(-c3ccc4c(c3)sc3ccc5ccccc5c34)cc2)c1 ; c1ccc(-c2ccc3c(ccc4cc(N(c5ccc(-c6ccc7c(ccc8ccccc87)c6)cc5)c5ccccc5-c5cccc(N(c6ccc(-c7cc8sc9ccccc9c8c8ccccc78)cc6)c6cc7ccccc7c7ccccc67)c5)ccc43)c2)cc1]

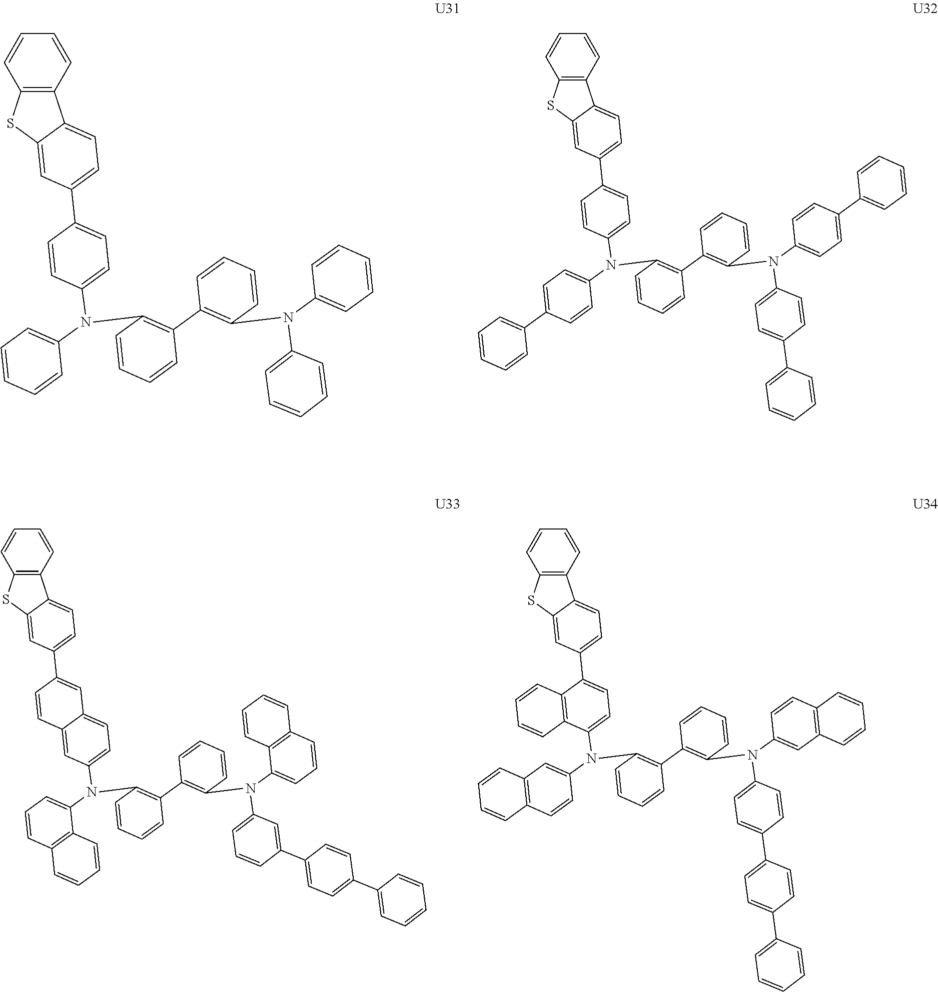 [SMILES: c1ccc(N(c2ccccc2)c2ccccc2-c2ccccc2N(c2ccccc2)c2ccc(-c3ccc4c(c3)sc3ccccc34)cc2)cc1 ; c1ccc(-c2ccc(N(c3ccc(-c4ccccc4)cc3)c3ccccc3-c3ccccc3N(c3ccc(-c4ccccc4)cc3)c3ccc(-c4ccc5c(c4)sc4ccccc45)cc3)cc2)cc1 ; c1ccc(-c2ccc(-c3cccc(N(c4ccccc4-c4ccccc4N(c4ccc5cc(-c6ccc7c(c6)sc6ccccc67)ccc5c4)c4cccc5ccccc45)c4cccc5ccccc45)c3)cc2)cc1 ; c1ccc(-c2ccc(-c3ccc(N(c4ccc5ccccc5c4)c4ccccc4-c4ccccc4N(c4ccc5ccccc5c4)c4ccc(-c5ccc6c(c5)sc5ccccc56)c5ccccc45)cc3)cc2)cc1]

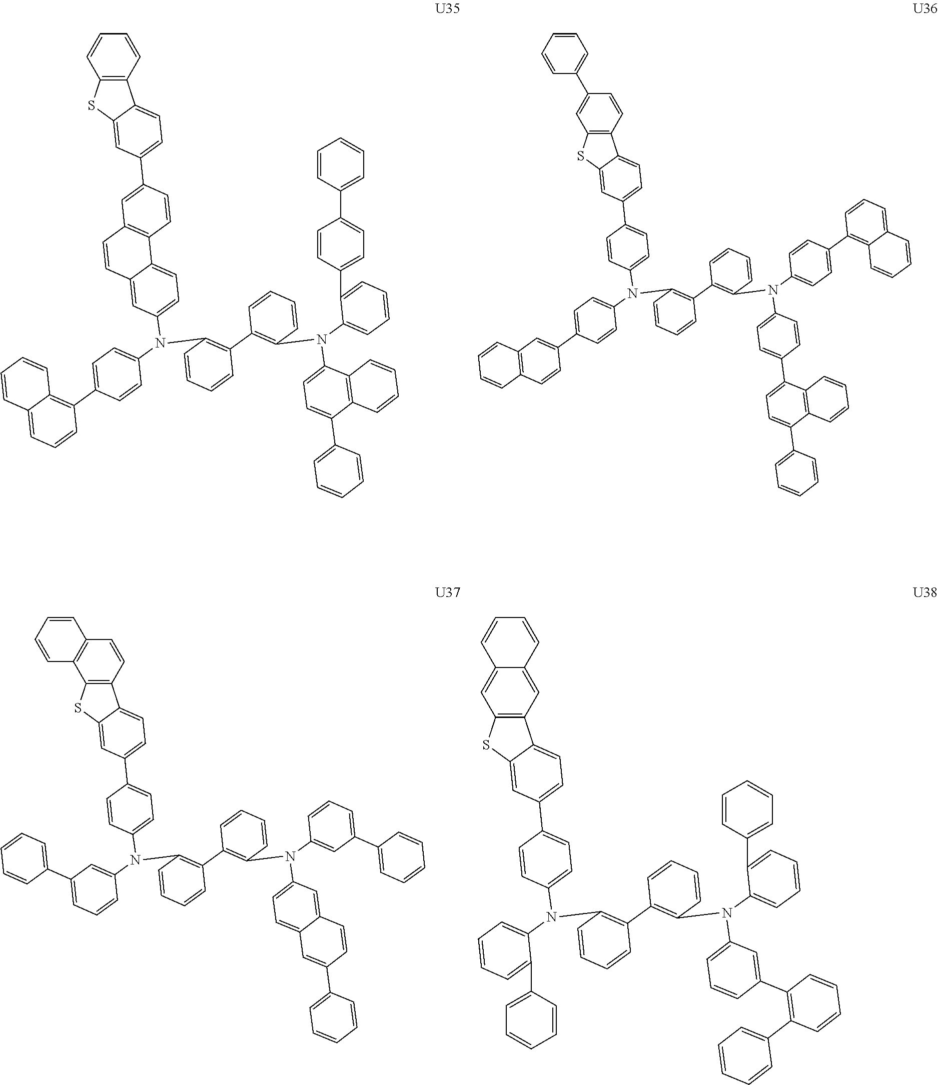

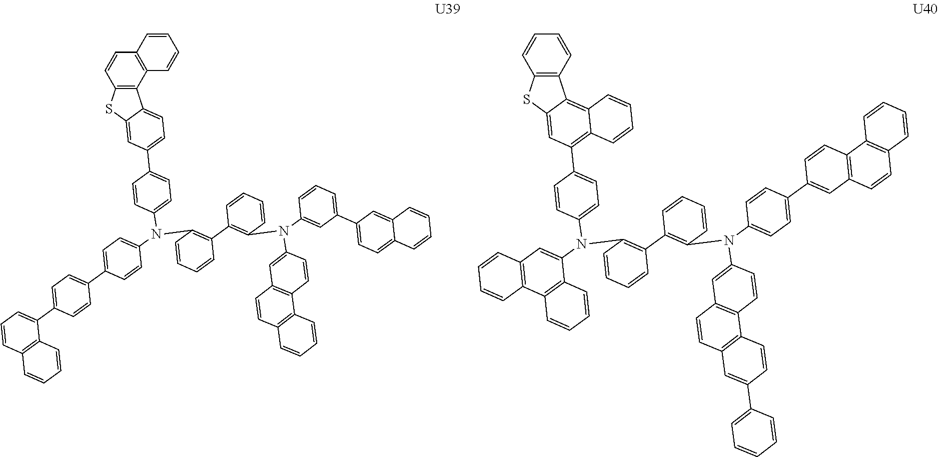 [SMILES: c1cc(-c2ccc3ccccc3c2)cc(N(c2ccc3c(ccc4ccccc43)c2)c2ccccc2-c2ccccc2N(c2ccc(-c3ccc(-c4cccc5ccccc45)cc3)cc2)c2ccc(-c3ccc4c(c3)sc3ccc5ccccc5c34)cc2)c1 ; c1ccc(-c2ccc3c(ccc4cc(N(c5ccc(-c6ccc7c(ccc8ccccc87)c6)cc5)c5ccccc5-c5ccccc5N(c5ccc(-c6cc7sc8ccccc8c7c7ccccc67)cc5)c5cc6ccccc6c6ccccc56)ccc43)c2)cc1]

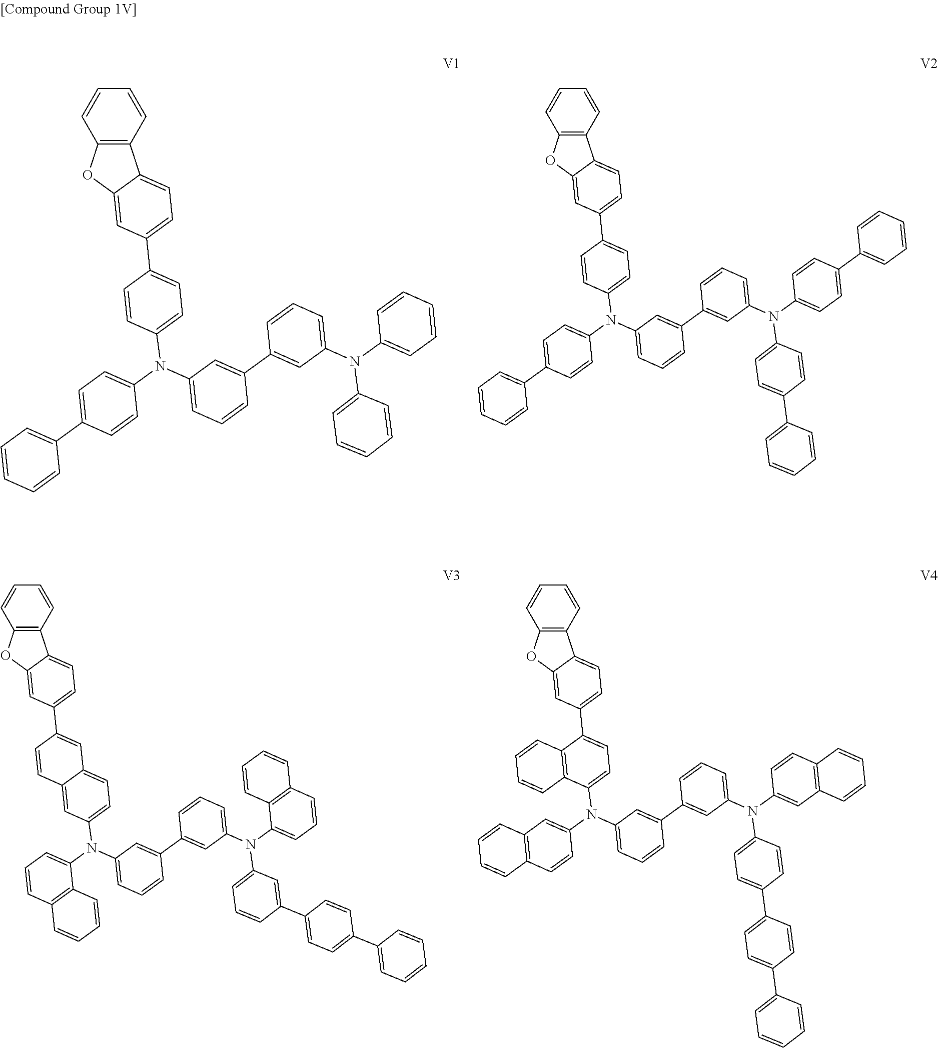 [SMILES: c1ccc(-c2ccc(N(c3ccc(-c4ccc5c(c4)oc4ccccc45)cc3)c3cccc(-c4cccc(N(c5ccccc5)c5ccccc5)c4)c3)cc2)cc1 ; c1ccc(-c2ccc(N(c3ccc(-c4ccccc4)cc3)c3cccc(-c4cccc(N(c5ccc(-c6ccccc6)cc5)c5ccc(-c6ccc7c(c6)oc6ccccc67)cc5)c4)c3)cc2)cc1 ; c1ccc(-c2ccc(-c3cccc(N(c4cccc(-c5cccc(N(c6ccc7cc(-c8ccc9c(c8)oc8ccccc89)ccc7c6)c6cccc7ccccc67)c5)c4)c4cccc5ccccc45)c3)cc2)cc1 ; c1ccc(-c2ccc(-c3ccc(N(c4cccc(-c5cccc(N(c6ccc7ccccc7c6)c6ccc(-c7ccc8c(c7)oc7ccccc78)c7ccccc67)c5)c4)c4ccc5ccccc5c4)cc3)cc2)cc1]

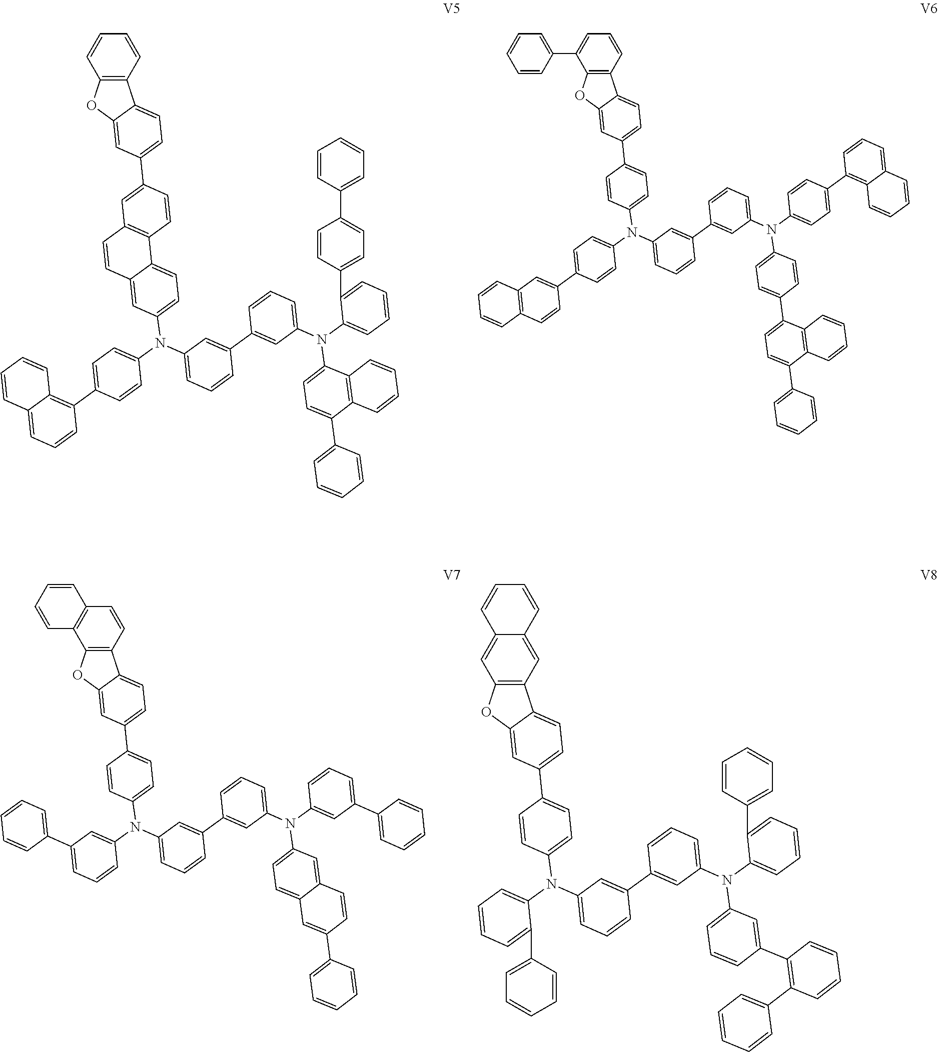

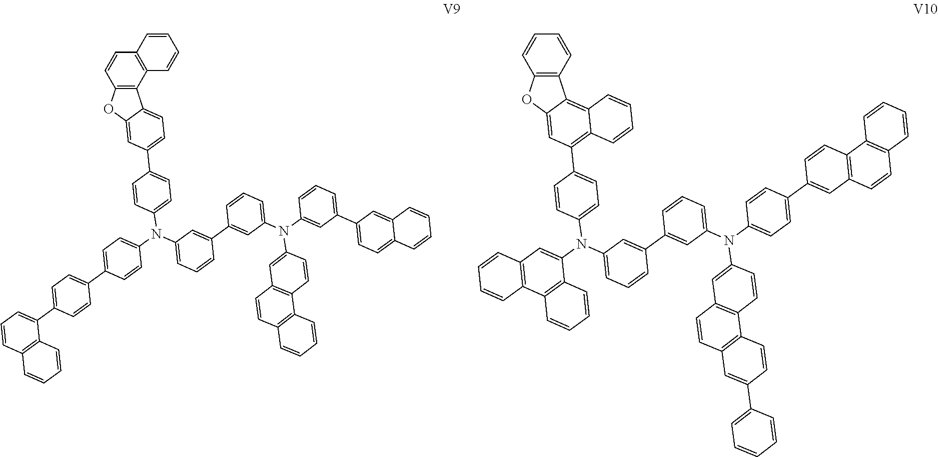 [SMILES: c1cc(-c2cccc(N(c3cccc(-c4ccc5ccccc5c4)c3)c3ccc4c(ccc5ccccc54)c3)c2)cc(N(c2ccc(-c3ccc(-c4cccc5ccccc45)cc3)cc2)c2ccc(-c3ccc4c(c3)oc3ccc5ccccc5c34)cc2)c1 ; c1ccc(-c2ccc3c(ccc4cc(N(c5ccc(-c6ccc7c(ccc8ccccc87)c6)cc5)c5cccc(-c6cccc(N(c7ccc(-c8cc9oc%10ccccc%10c9c9ccccc89)cc7)c7cc8ccccc8c8ccccc78)c6)c5)ccc43)c2)cc1]

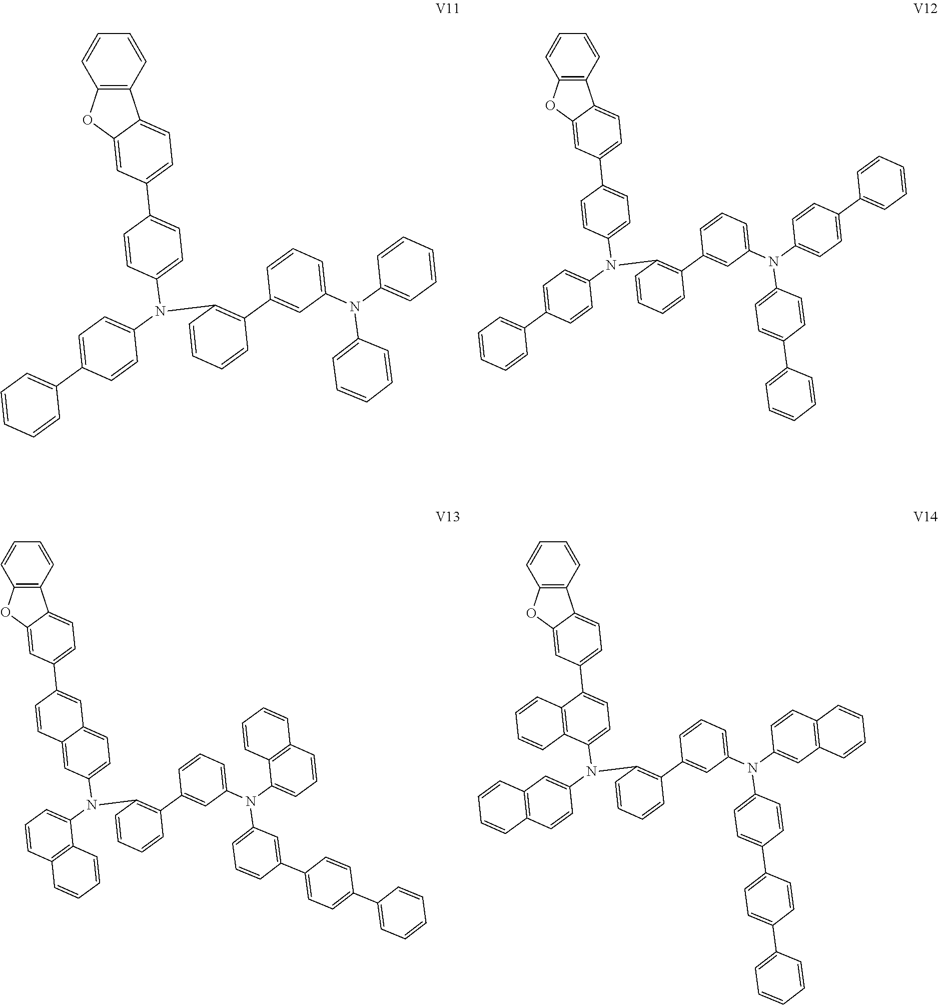 [SMILES: c1ccc(-c2ccc(N(c3ccc(-c4ccc5c(c4)oc4ccccc45)cc3)c3ccccc3-c3cccc(N(c4ccccc4)c4ccccc4)c3)cc2)cc1 ; c1ccc(-c2ccc(N(c3ccc(-c4ccccc4)cc3)c3cccc(-c4ccccc4N(c4ccc(-c5ccccc5)cc4)c4ccc(-c5ccc6c(c5)oc5ccccc56)cc4)c3)cc2)cc1 ; c1ccc(-c2ccc(-c3cccc(N(c4cccc(-c5ccccc5N(c5ccc6cc(-c7ccc8c(c7)oc7ccccc78)ccc6c5)c5cccc6ccccc56)c4)c4cccc5ccccc45)c3)cc2)cc1 ; c1ccc(-c2ccc(-c3ccc(N(c4cccc(-c5ccccc5N(c5ccc6ccccc6c5)c5ccc(-c6ccc7c(c6)oc6ccccc67)c6ccccc56)c4)c4ccc5ccccc5c4)cc3)cc2)cc1]

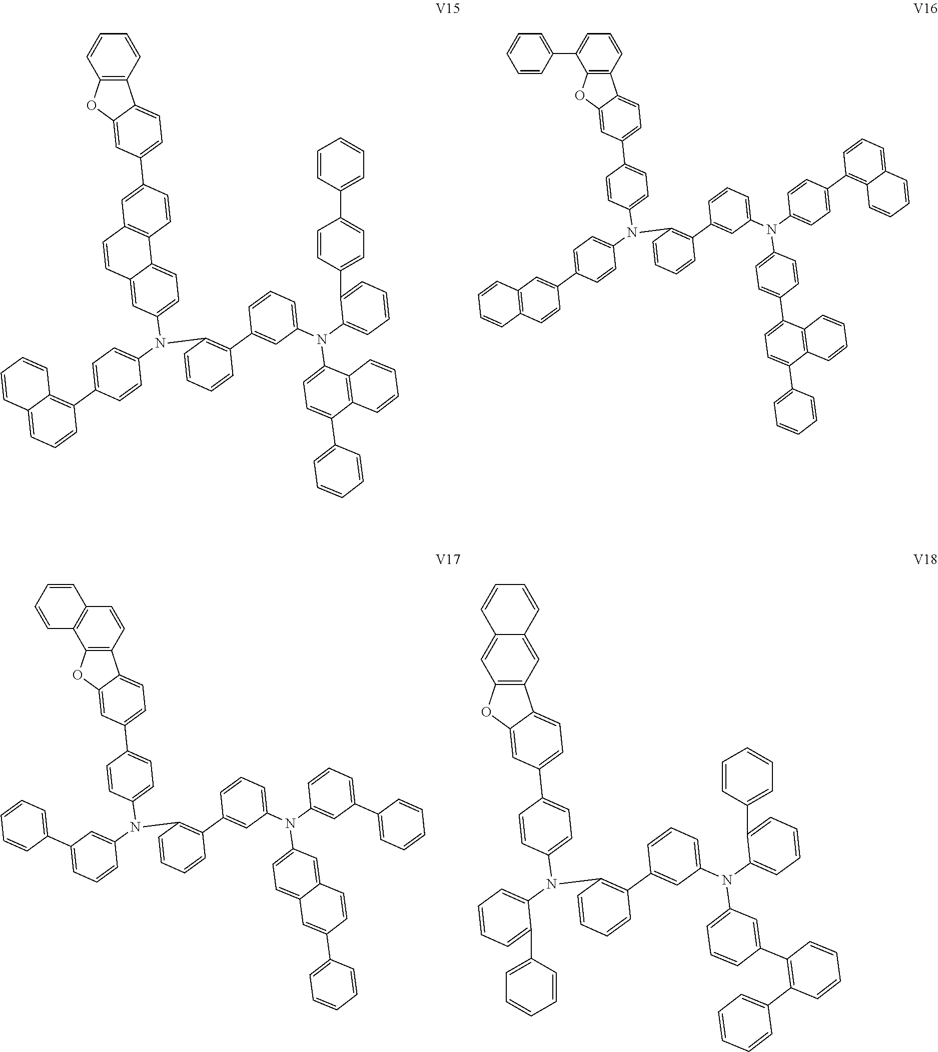

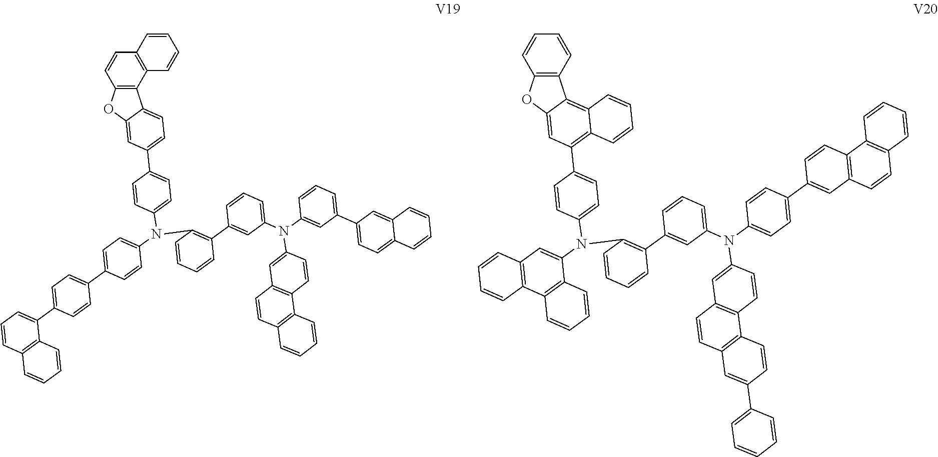 [SMILES: c1cc(-c2ccc3ccccc3c2)cc(N(c2cccc(-c3ccccc3N(c3ccc(-c4ccc(-c5cccc6ccccc56)cc4)cc3)c3ccc(-c4ccc5c(c4)oc4ccc6ccccc6c45)cc3)c2)c2ccc3c(ccc4ccccc43)c2)c1 ; c1ccc(-c2ccc3c(ccc4cc(N(c5ccc(-c6ccc7c(ccc8ccccc87)c6)cc5)c5cccc(-c6ccccc6N(c6ccc(-c7cc8oc9ccccc9c8c8ccccc78)cc6)c6cc7ccccc7c7ccccc67)c5)ccc43)c2)cc1]

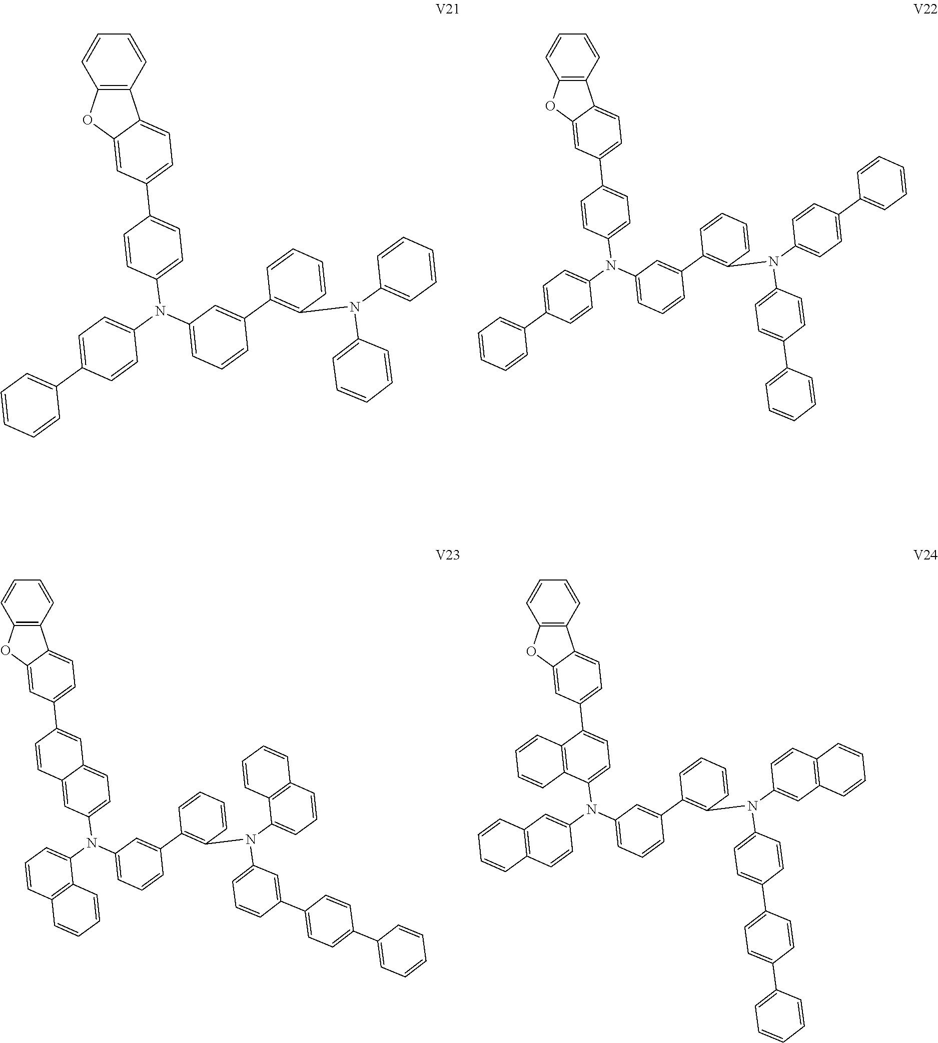 [SMILES: c1ccc(-c2ccc(N(c3ccc(-c4ccc5c(c4)oc4ccccc45)cc3)c3cccc(-c4ccccc4N(c4ccccc4)c4ccccc4)c3)cc2)cc1 ; c1ccc(-c2ccc(N(c3ccc(-c4ccc5c(c4)oc4ccccc45)cc3)c3cccc(-c4ccccc4N(c4ccc(-c5ccccc5)cc4)c4ccc(-c5ccccc5)cc4)c3)cc2)cc1 ; c1ccc(-c2ccc(-c3cccc(N(c4ccccc4-c4cccc(N(c5ccc6cc(-c7ccc8c(c7)oc7ccccc78)ccc6c5)c5cccc6ccccc56)c4)c4cccc5ccccc45)c3)cc2)cc1 ; c1ccc(-c2ccc(-c3ccc(N(c4ccc5ccccc5c4)c4ccccc4-c4cccc(N(c5ccc6ccccc6c5)c5ccc(-c6ccc7c(c6)oc6ccccc67)c6ccccc56)c4)cc3)cc2)cc1]

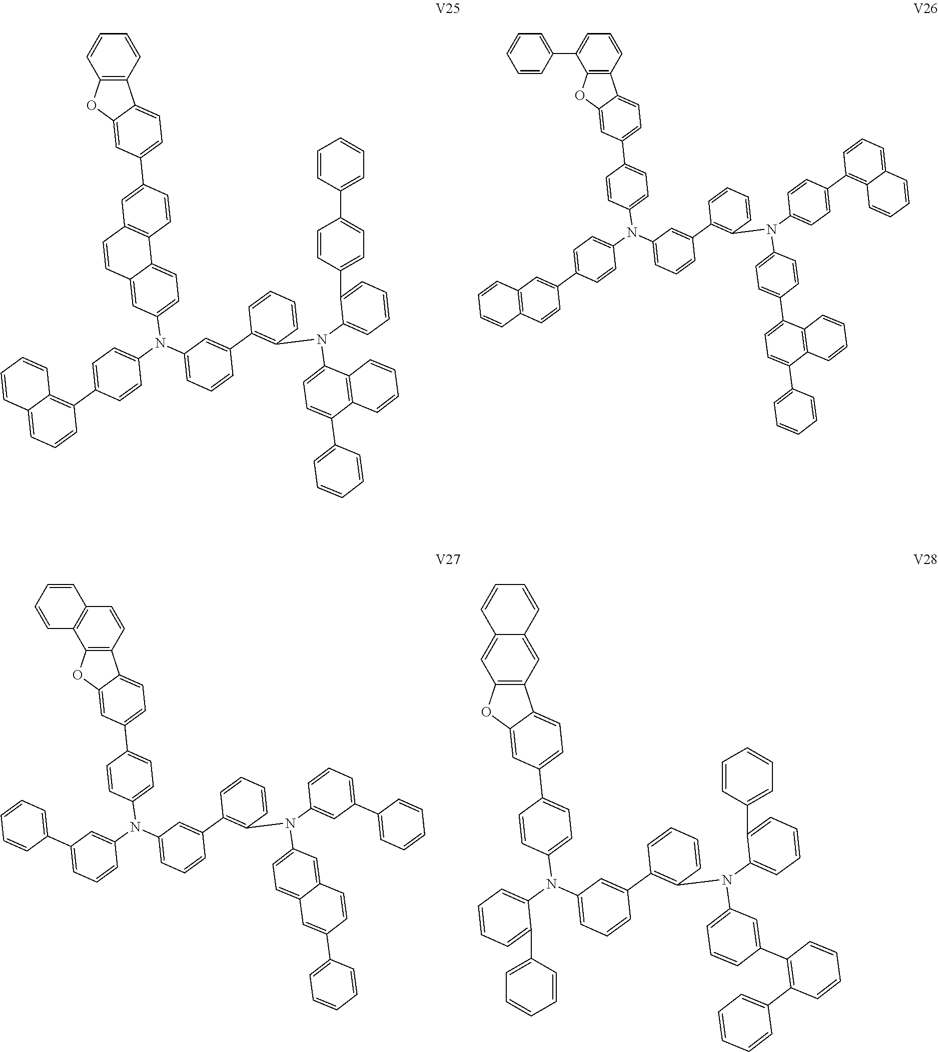

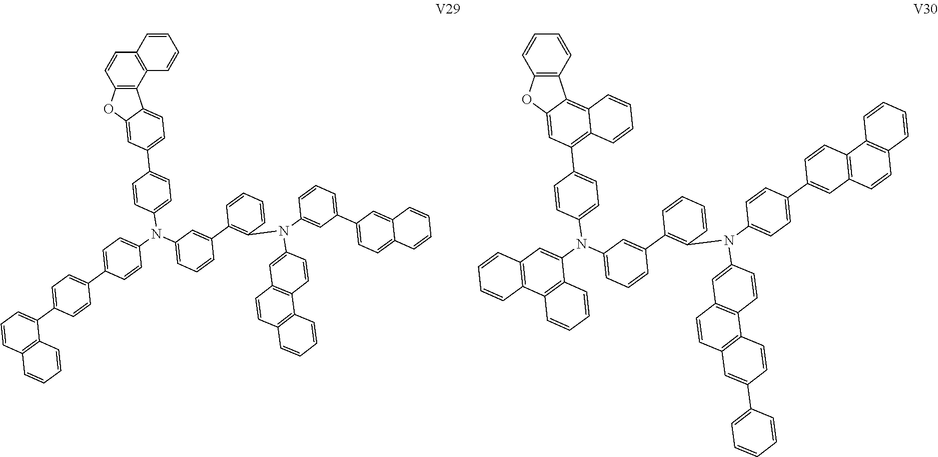 [SMILES: c1cc(-c2ccccc2N(c2cccc(-c3ccc4ccccc4c3)c2)c2ccc3c(ccc4ccccc43)c2)cc(N(c2ccc(-c3ccc(-c4cccc5ccccc45)cc3)cc2)c2ccc(-c3ccc4c(c3)oc3ccc5ccccc5c34)cc2)c1 ; c1ccc(-c2ccc3c(ccc4cc(N(c5ccc(-c6ccc7c(ccc8ccccc87)c6)cc5)c5ccccc5-c5cccc(N(c6ccc(-c7cc8oc9ccccc9c8c8ccccc78)cc6)c6cc7ccccc7c7ccccc67)c5)ccc43)c2)cc1]

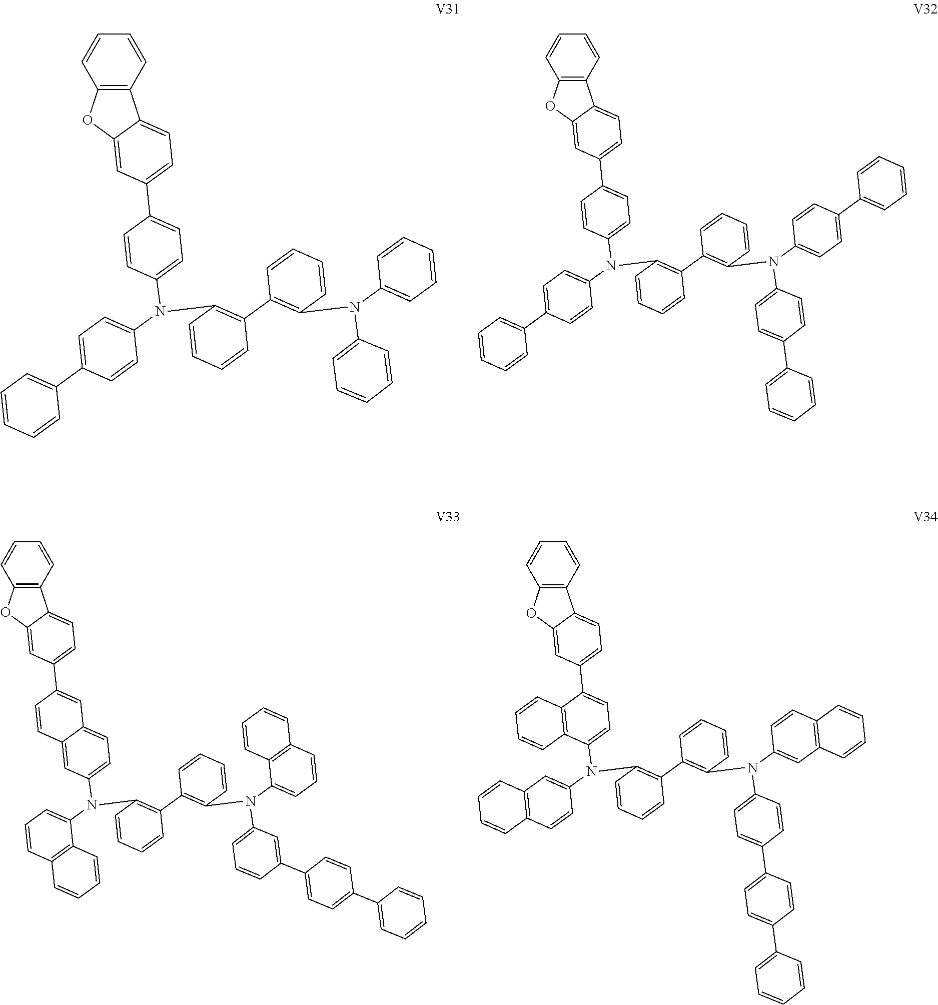 [SMILES: c1ccc(-c2ccc(N(c3ccc(-c4ccc5c(c4)oc4ccccc45)cc3)c3ccccc3-c3ccccc3N(c3ccccc3)c3ccccc3)cc2)cc1 ; c1ccc(-c2ccc(N(c3ccc(-c4ccccc4)cc3)c3ccccc3-c3ccccc3N(c3ccc(-c4ccccc4)cc3)c3ccc(-c4ccc5c(c4)oc4ccccc45)cc3)cc2)cc1 ; c1ccc(-c2ccc(-c3cccc(N(c4ccccc4-c4ccccc4N(c4ccc5cc(-c6ccc7c(c6)oc6ccccc67)ccc5c4)c4cccc5ccccc45)c4cccc5ccccc45)c3)cc2)cc1 ; c1ccc(-c2ccc(-c3ccc(N(c4ccc5ccccc5c4)c4ccccc4-c4ccccc4N(c4ccc5ccccc5c4)c4ccc(-c5ccc6c(c5)oc5ccccc56)c5ccccc45)cc3)cc2)cc1]

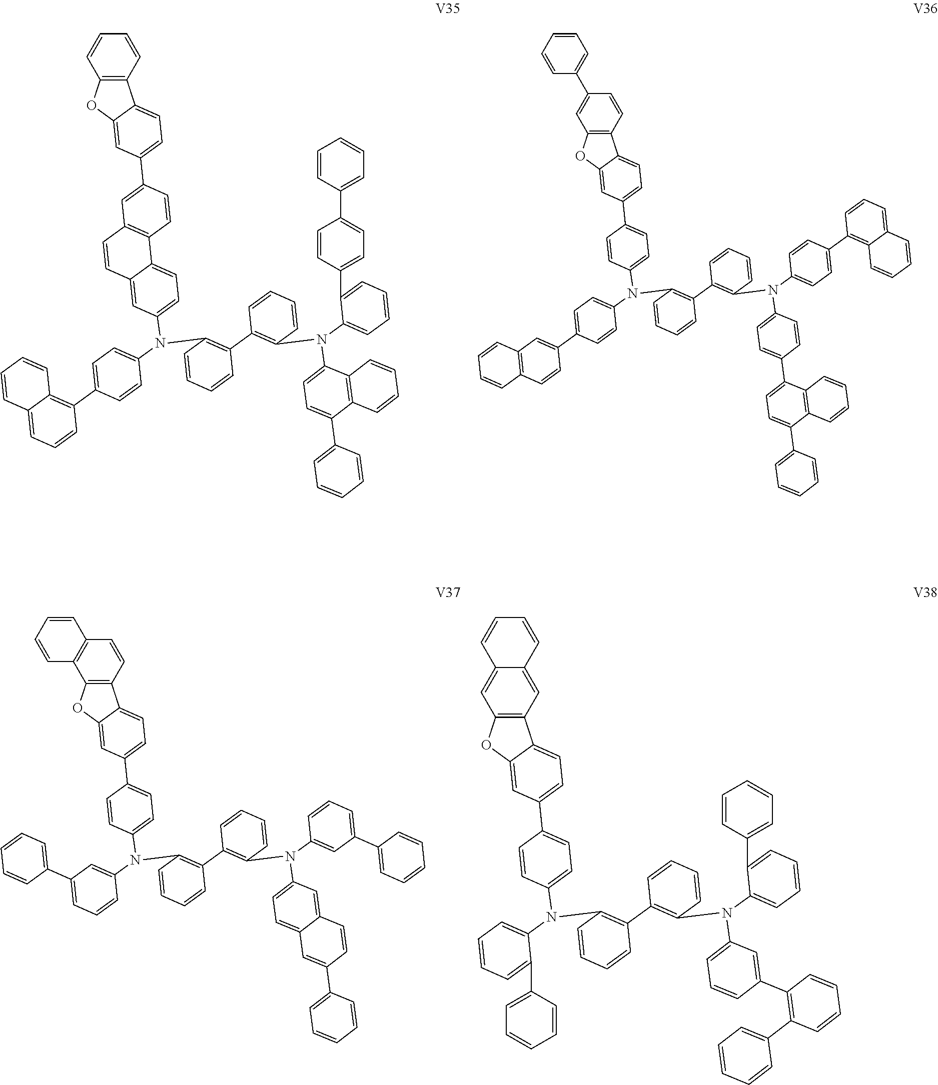

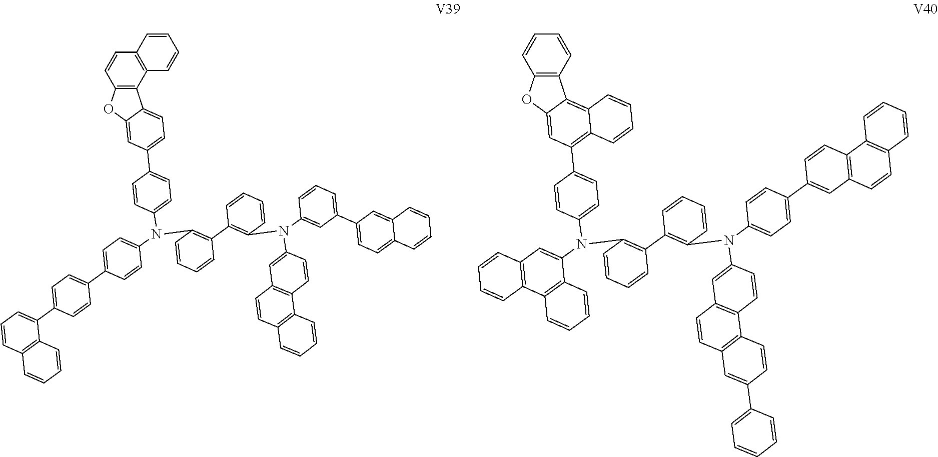 [SMILES: c1cc(-c2ccc3ccccc3c2)cc(N(c2ccc3c(ccc4ccccc43)c2)c2ccccc2-c2ccccc2N(c2ccc(-c3ccc(-c4cccc5ccccc45)cc3)cc2)c2ccc(-c3ccc4c(c3)oc3ccc5ccccc5c34)cc2)c1 ; c1ccc(-c2ccc3c(ccc4cc(N(c5ccc(-c6ccc7c(ccc8ccccc87)c6)cc5)c5ccccc5-c5ccccc5N(c5ccc(-c6cc7oc8ccccc8c7c7ccccc67)cc5)c5cc6ccccc6c6ccccc56)ccc43)c2)cc1]

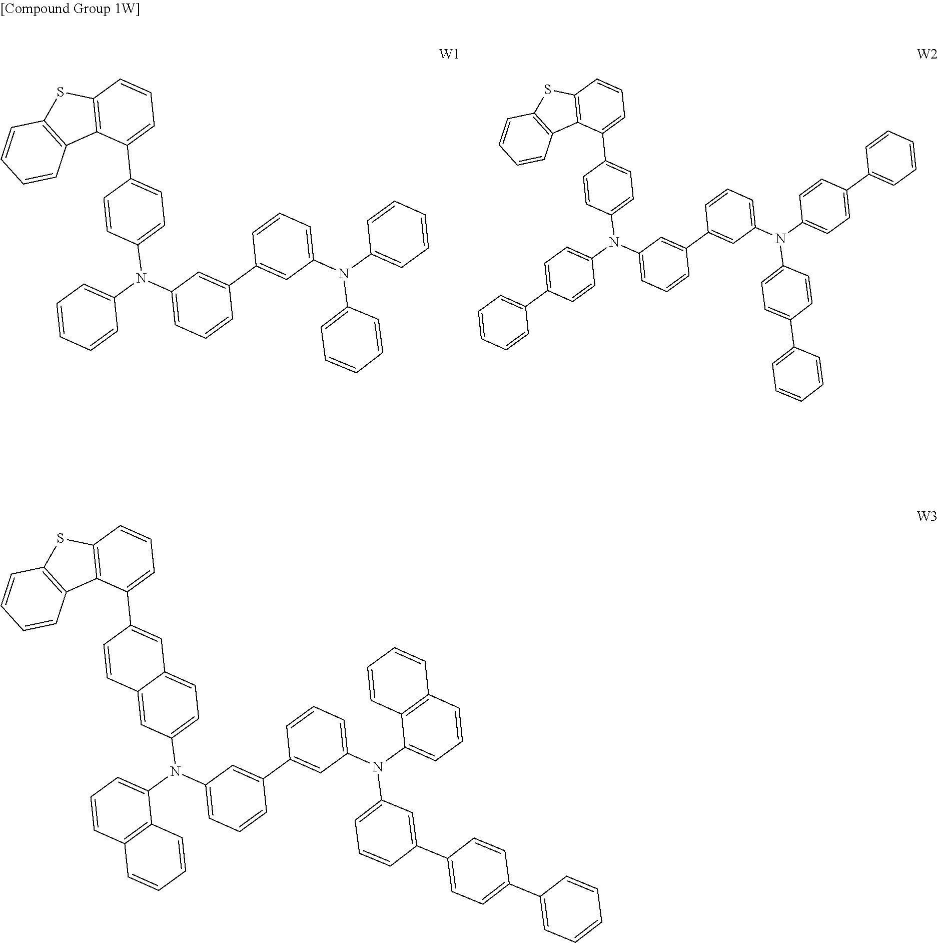 [SMILES: c1ccc(N(c2ccccc2)c2cccc(-c3cccc(N(c4ccccc4)c4ccc(-c5cccc6sc7ccccc7c56)cc4)c3)c2)cc1 ; c1ccc(-c2ccc(N(c3ccc(-c4ccccc4)cc3)c3cccc(-c4cccc(N(c5ccc(-c6ccccc6)cc5)c5ccc(-c6cccc7sc8ccccc8c67)cc5)c4)c3)cc2)cc1 ; c1ccc(-c2ccc(-c3cccc(N(c4cccc(-c5cccc(N(c6ccc7cc(-c8cccc9sc%10ccccc%10c89)ccc7c6)c6cccc7ccccc67)c5)c4)c4cccc5ccccc45)c3)cc2)cc1]

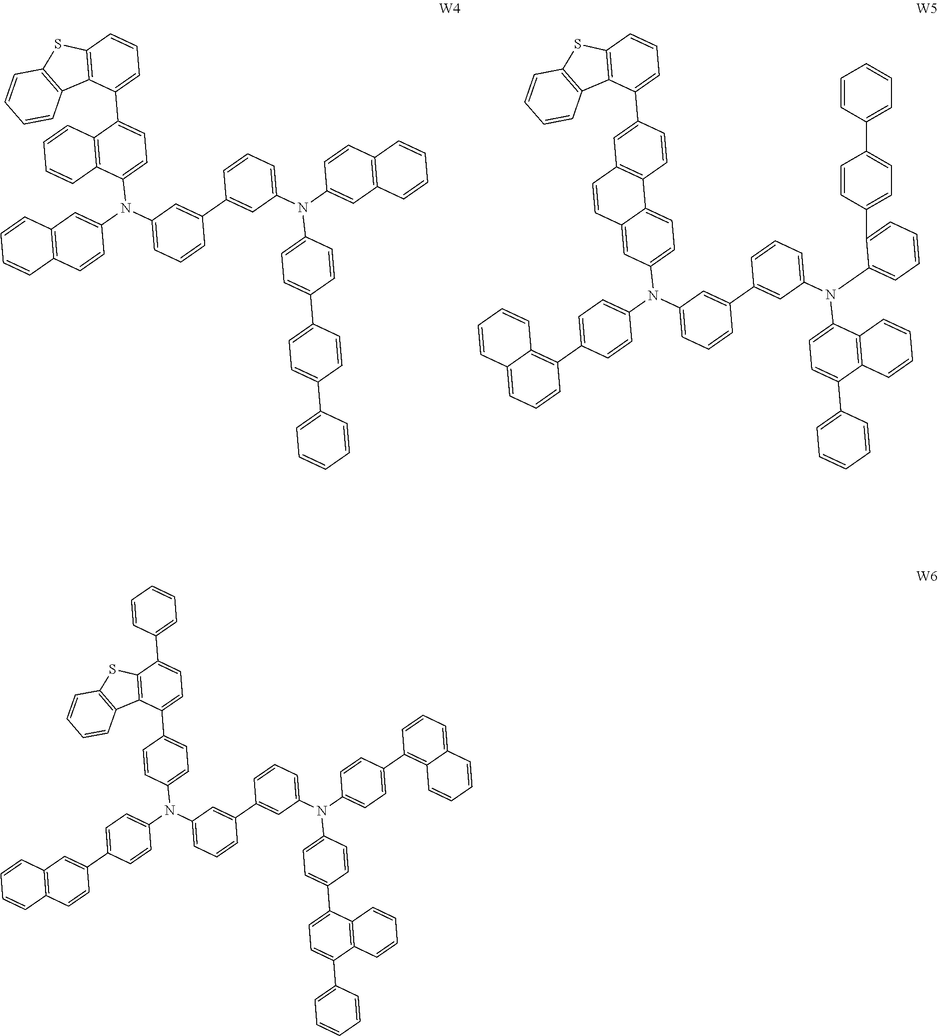 [SMILES: c1ccc(-c2ccc(-c3ccc(N(c4cccc(-c5cccc(N(c6ccc7ccccc7c6)c6ccc(-c7cccc8sc9ccccc9c78)c7ccccc67)c5)c4)c4ccc5ccccc5c4)cc3)cc2)cc1 ; c1ccc(-c2ccc(-c3ccccc3N(c3cccc(-c4cccc(N(c5ccc(-c6cccc7ccccc67)cc5)c5ccc6c(ccc7cc(-c8cccc9sc%10ccccc%10c89)ccc76)c5)c4)c3)c3ccc(-c4ccccc4)c4ccccc34)cc2)cc1 ; c1ccc(-c2ccc(-c3ccc(N(c4ccc(-c5cccc6ccccc56)cc4)c4cccc(-c5cccc(N(c6ccc(-c7ccc8ccccc8c7)cc6)c6ccc(-c7ccc(-c8ccccc8)c8sc9ccccc9c78)cc6)c5)c4)cc3)c3ccccc23)cc1]

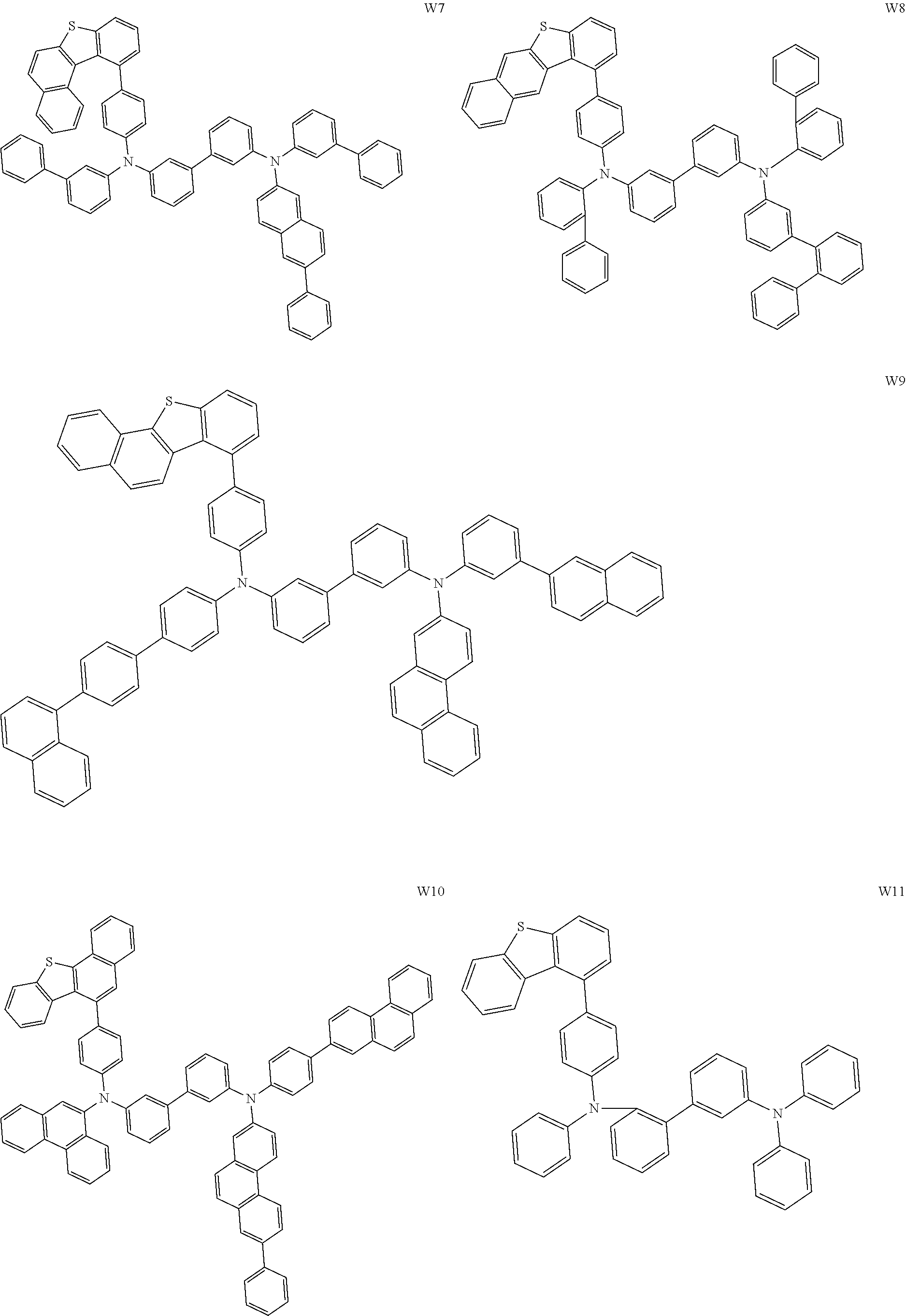

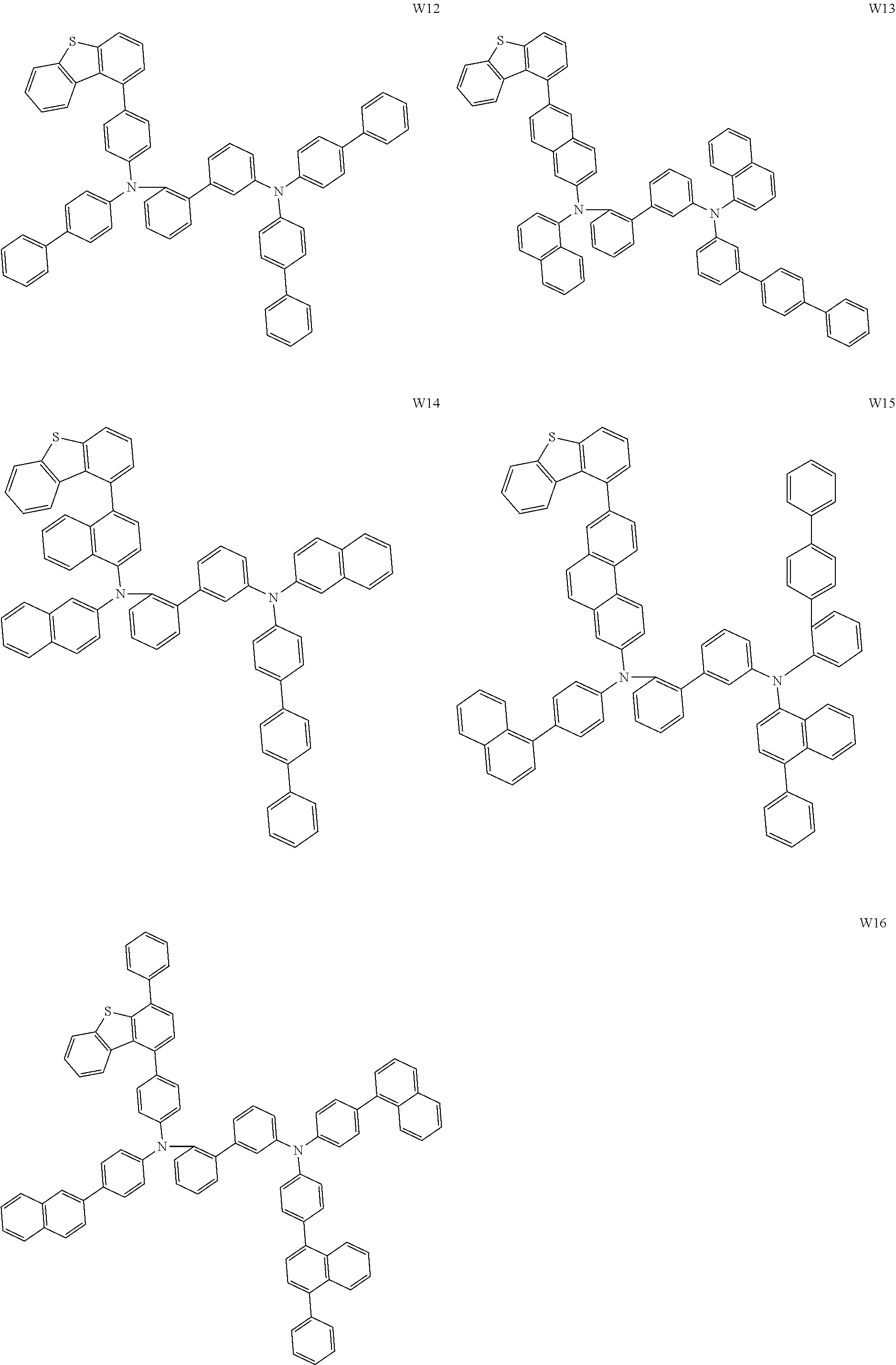

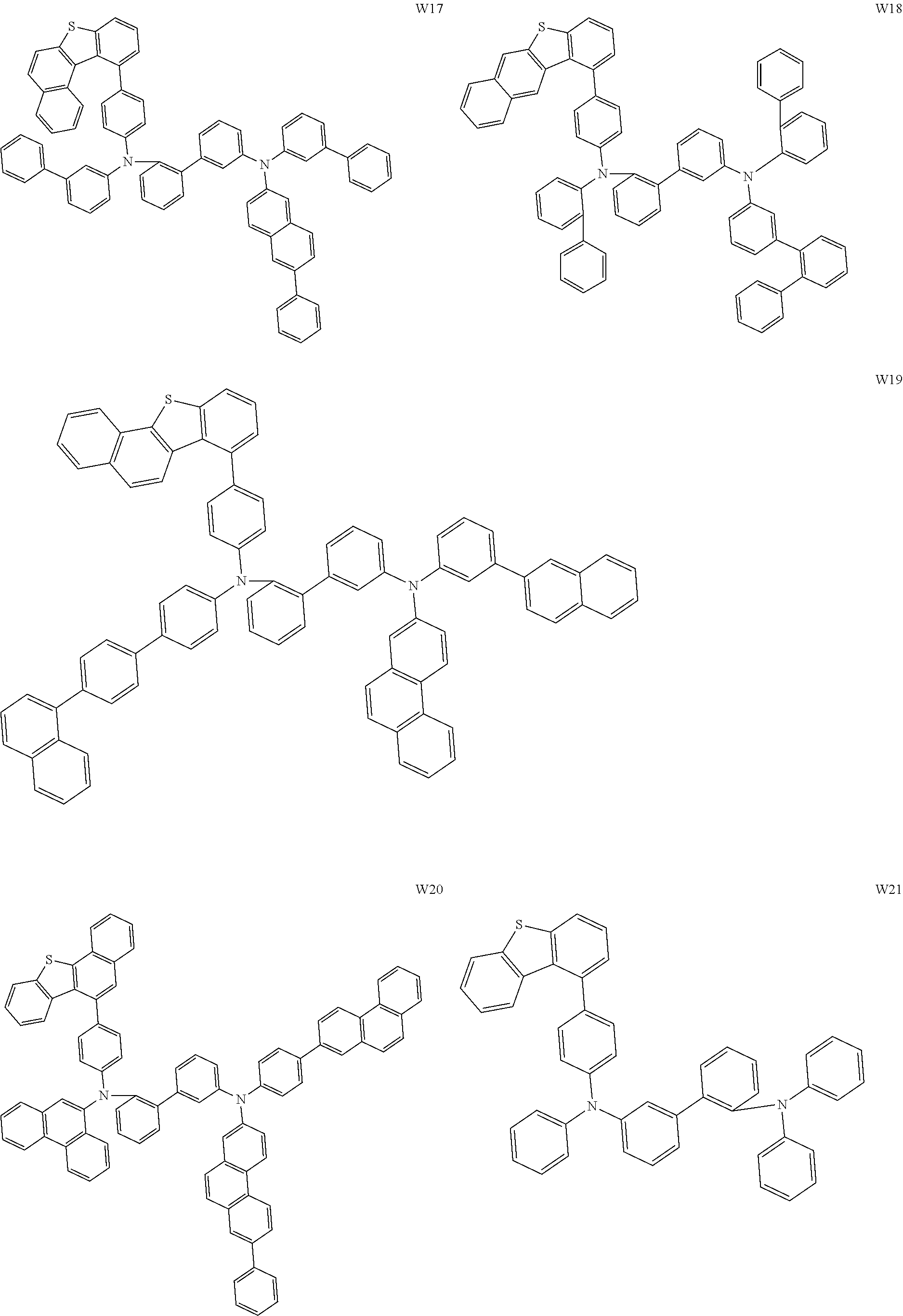

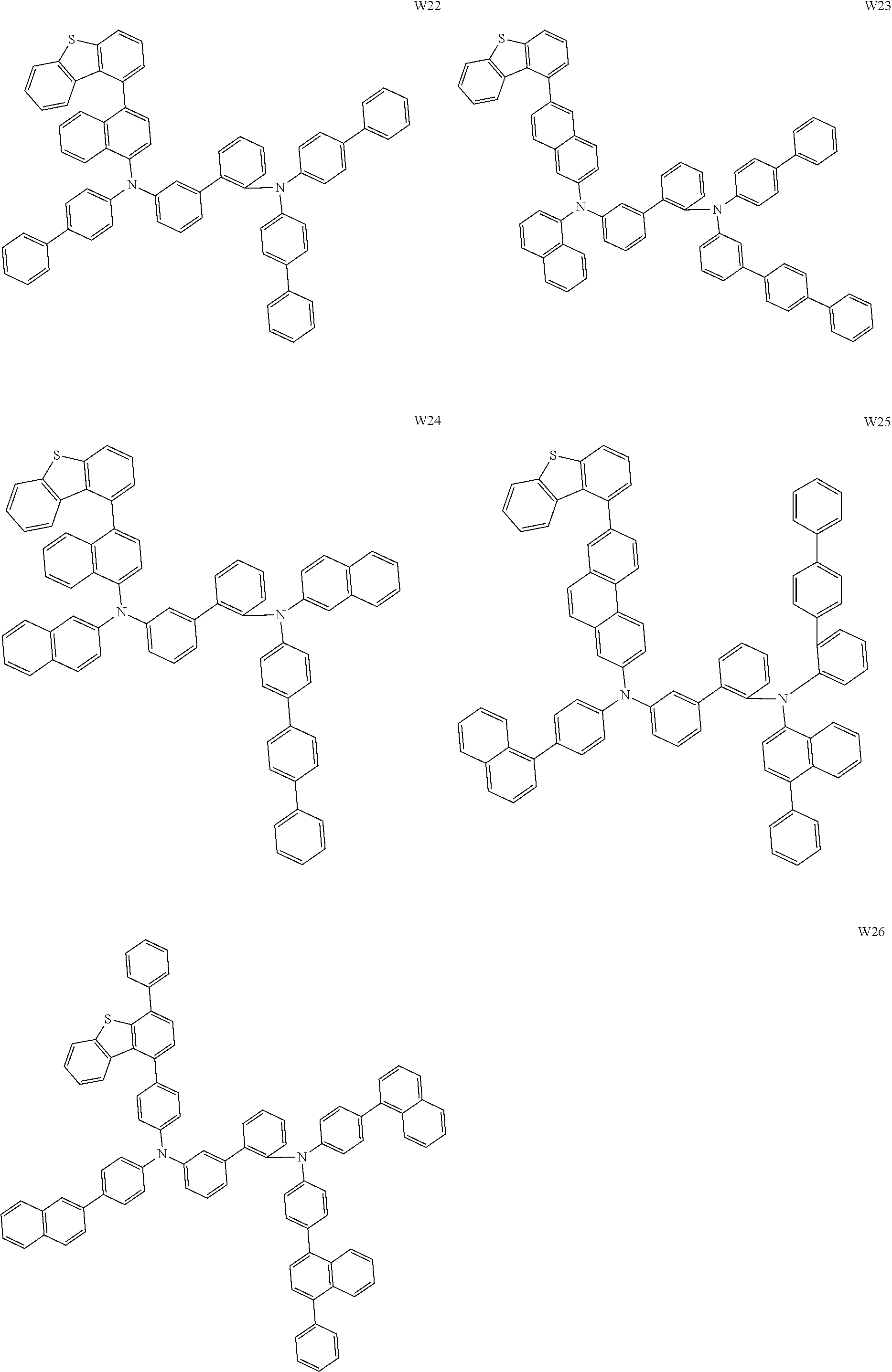

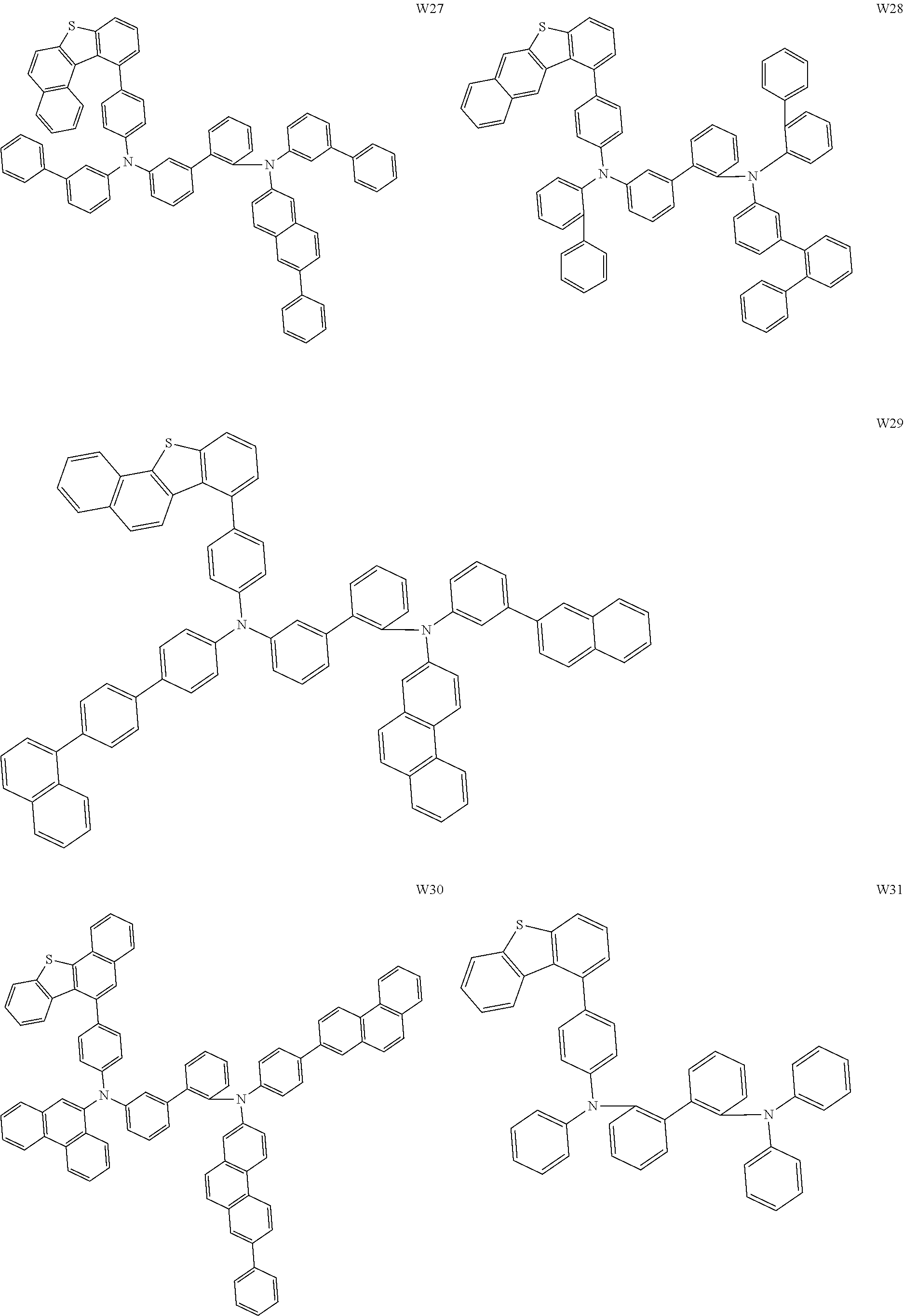

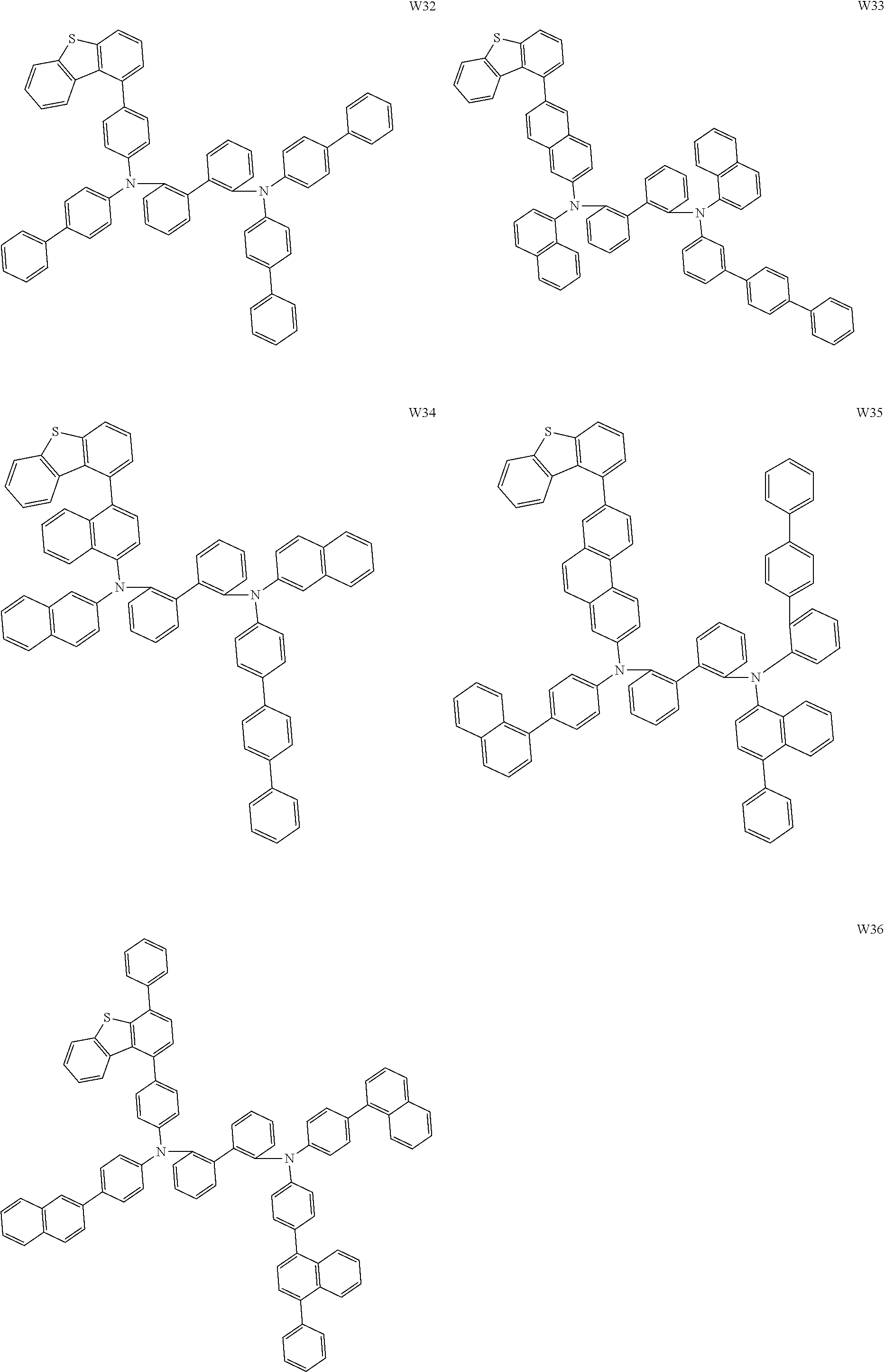

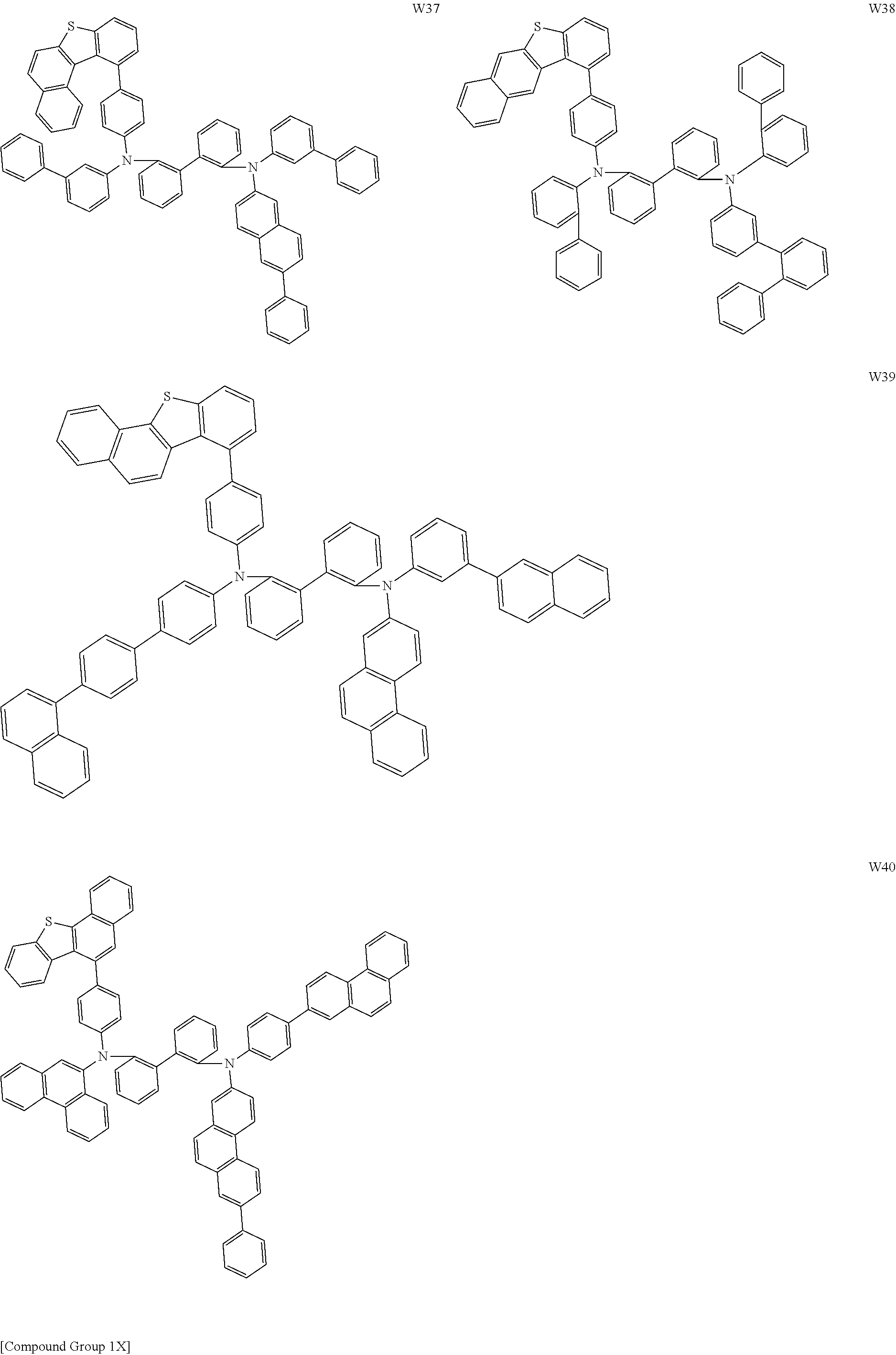

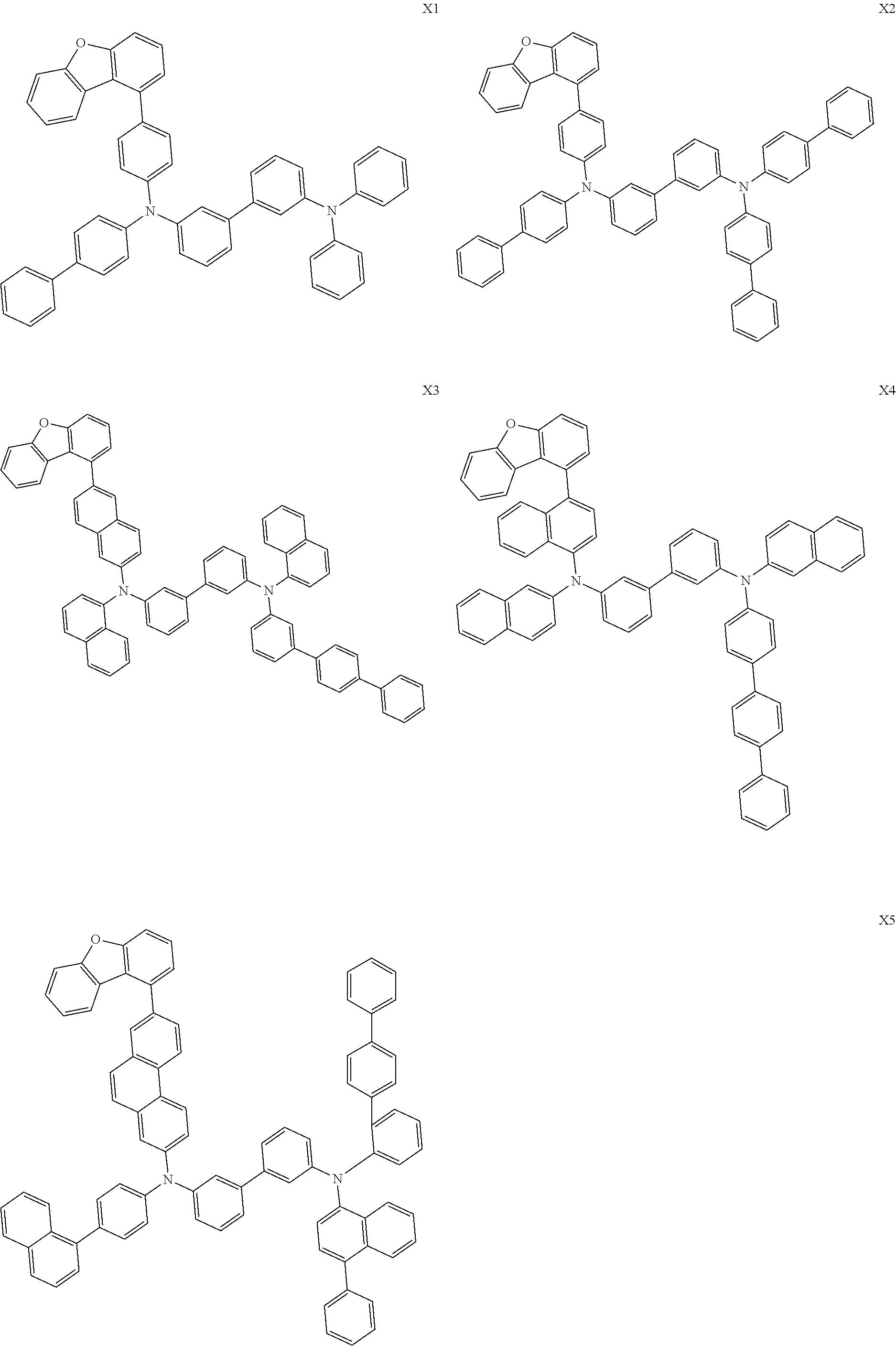

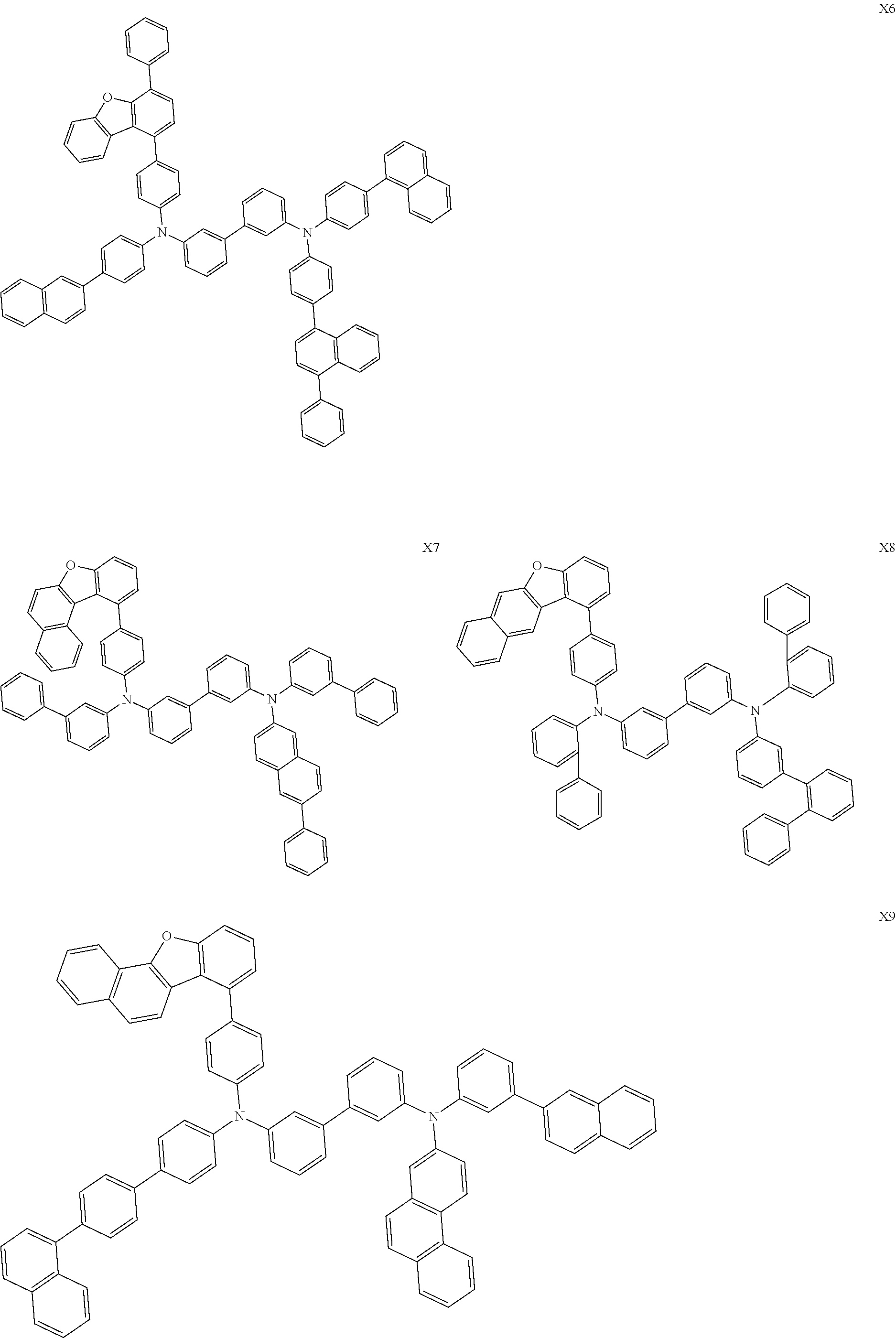

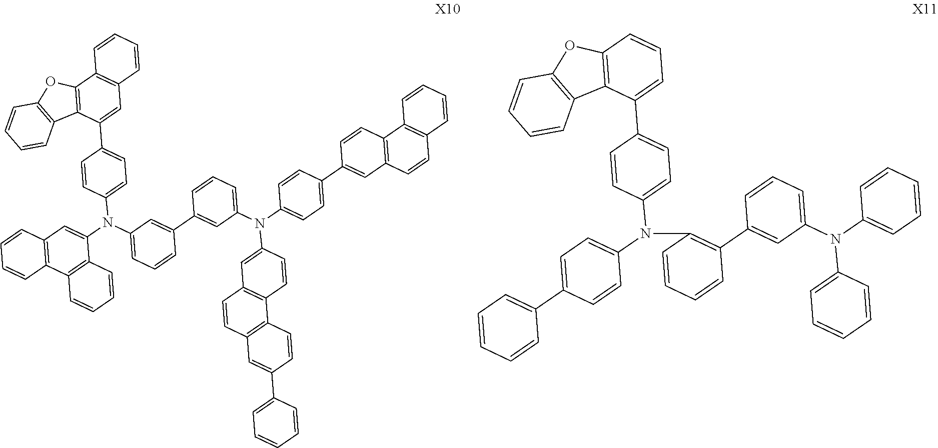 [SMILES: c1ccc(-c2ccc3c(ccc4cc(N(c5ccc(-c6ccc7c(ccc8ccccc87)c6)cc5)c5cccc(-c6cccc(N(c7ccc(-c8cc9ccccc9c9oc%10ccccc%10c89)cc7)c7cc8ccccc8c8ccccc78)c6)c5)ccc43)c2)cc1 ; c1ccc(-c2ccc(N(c3ccc(-c4cccc5oc6ccccc6c45)cc3)c3ccccc3-c3cccc(N(c4ccccc4)c4ccccc4)c3)cc2)cc1]

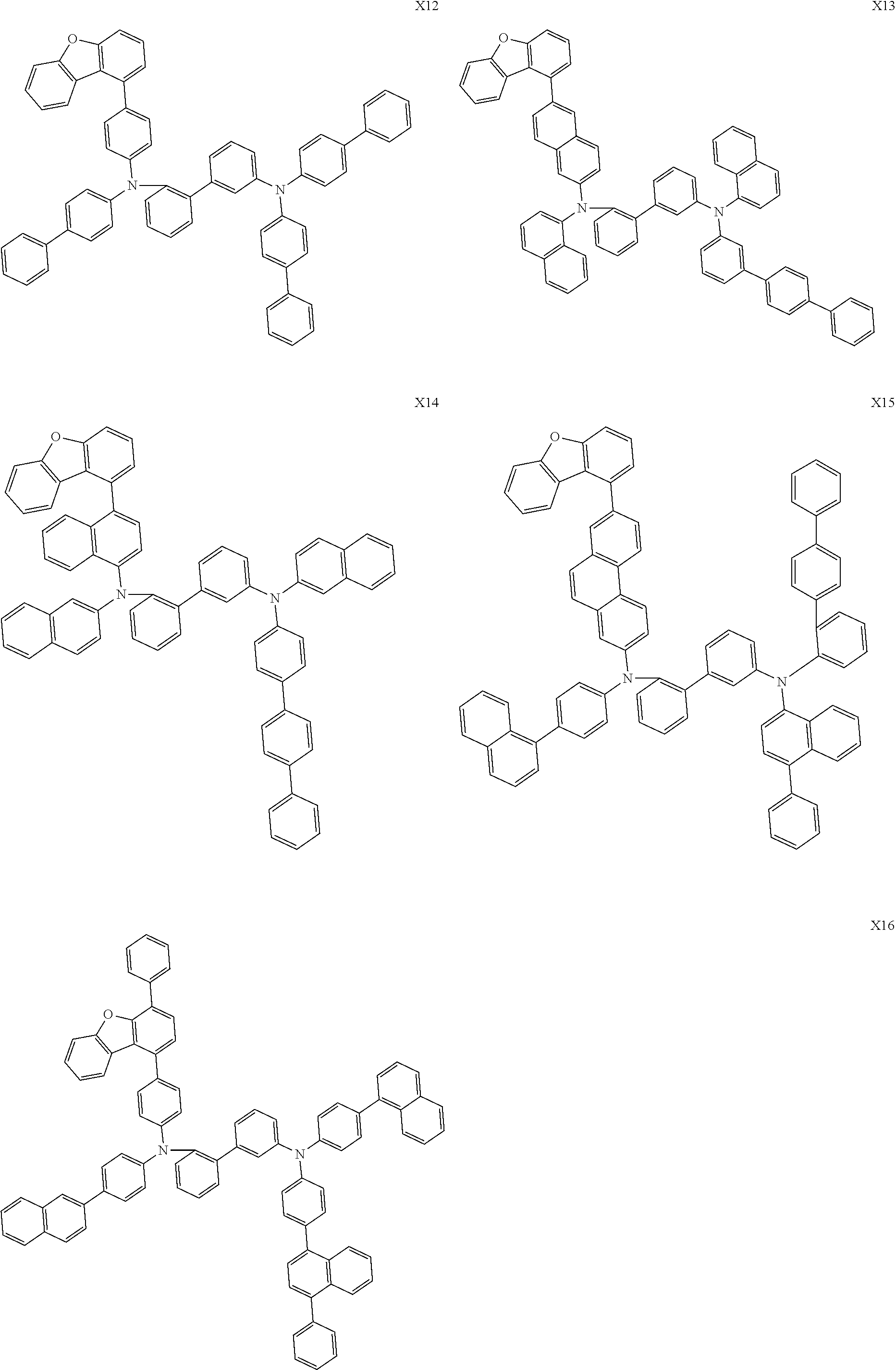

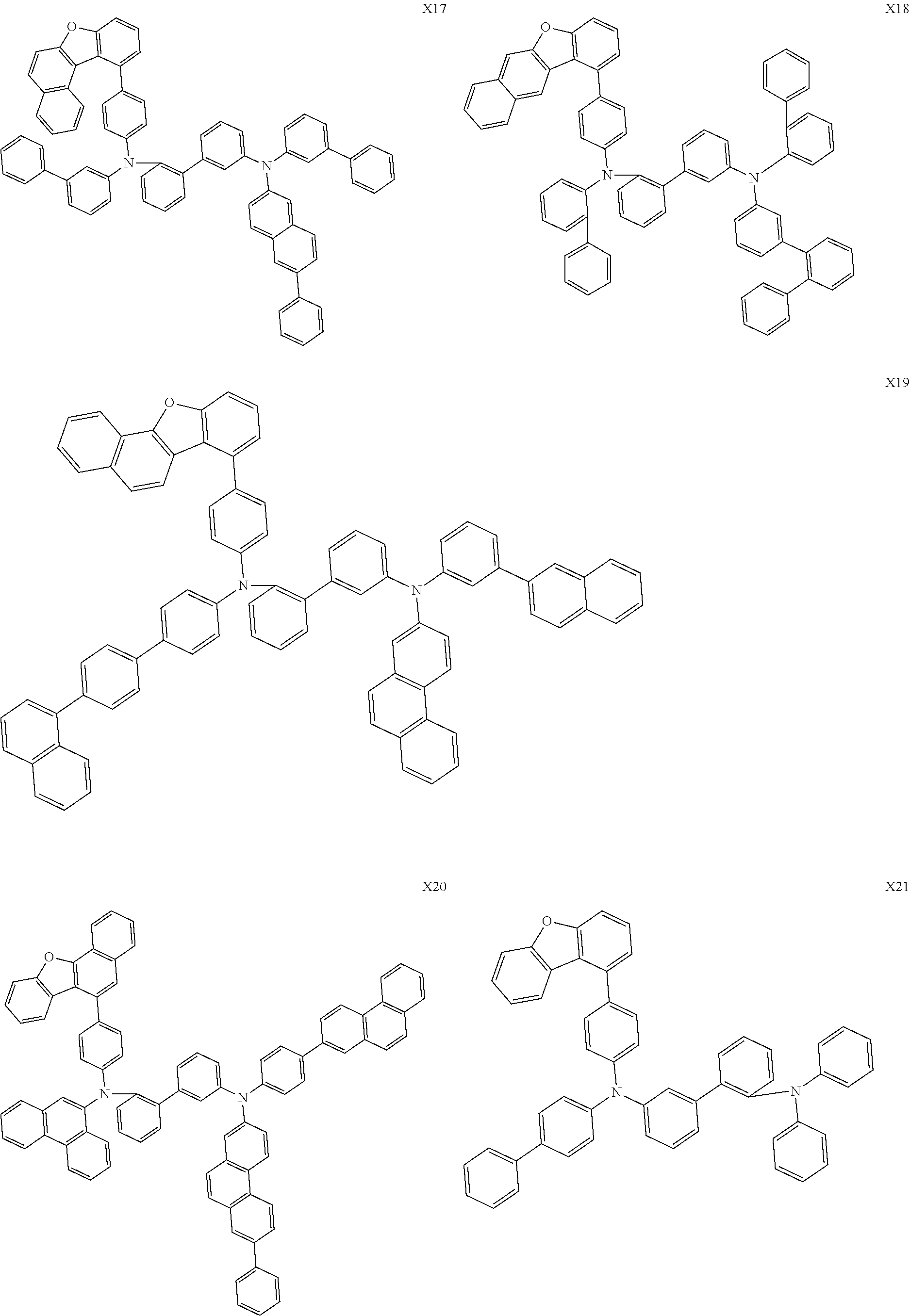

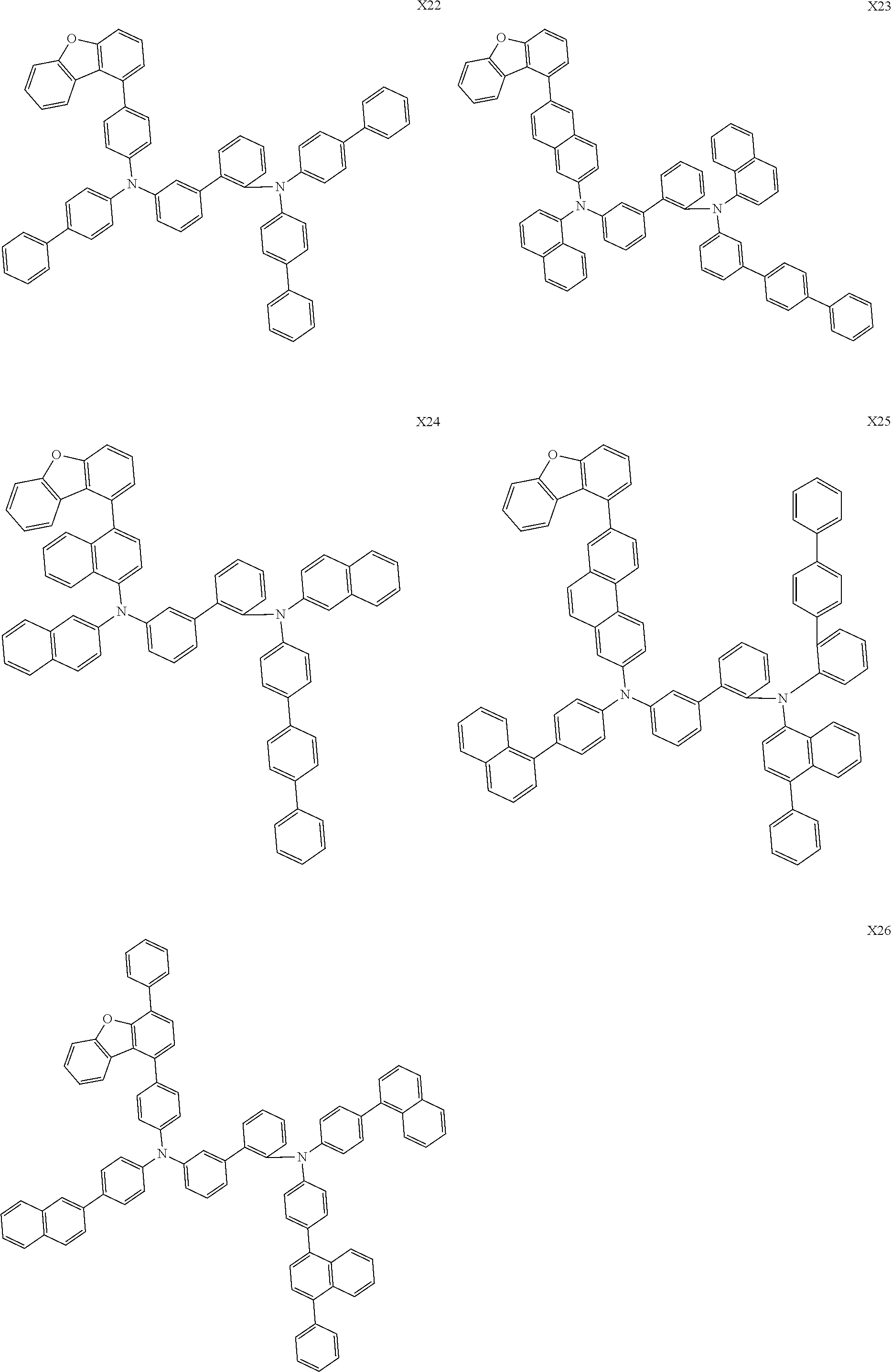

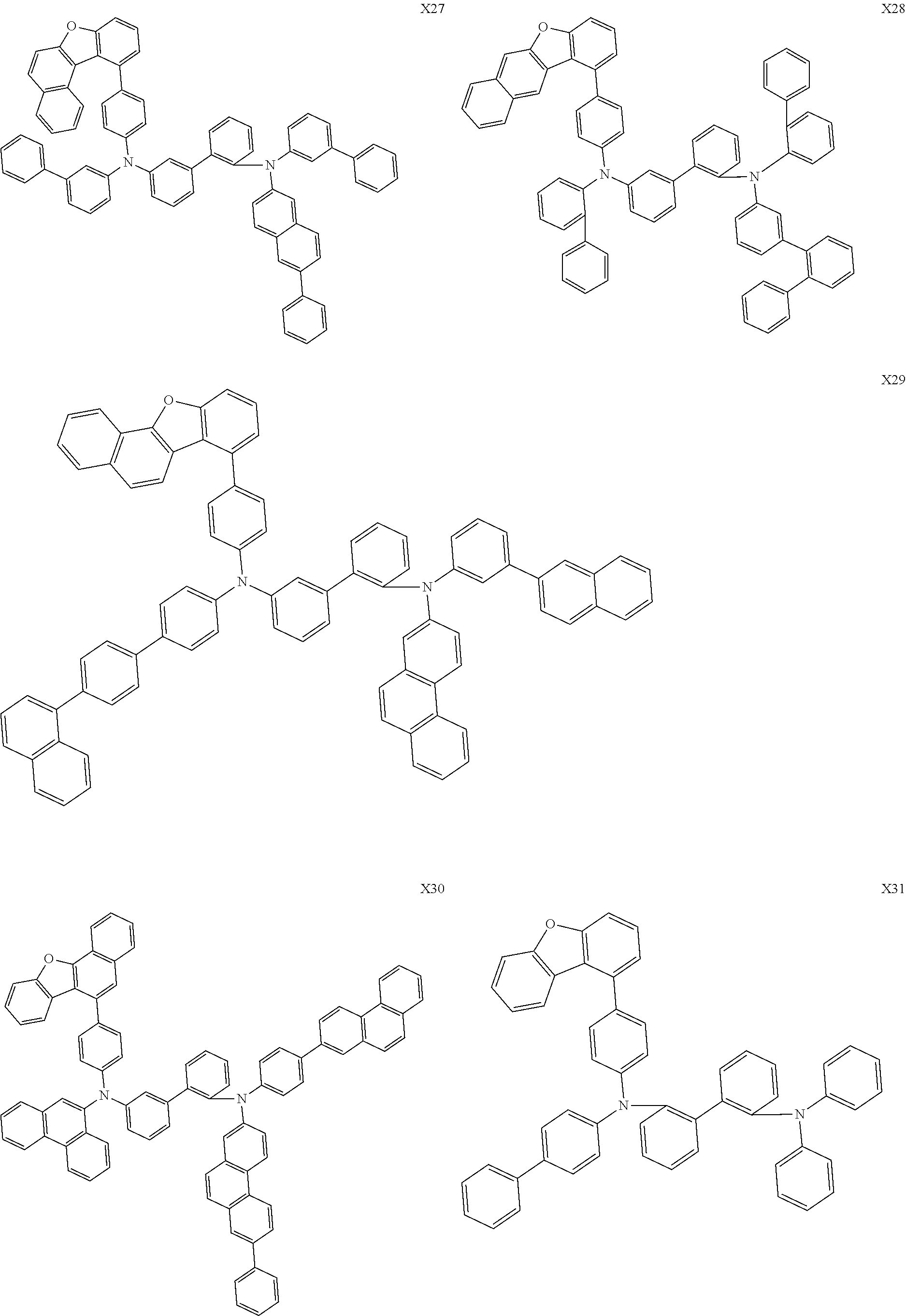

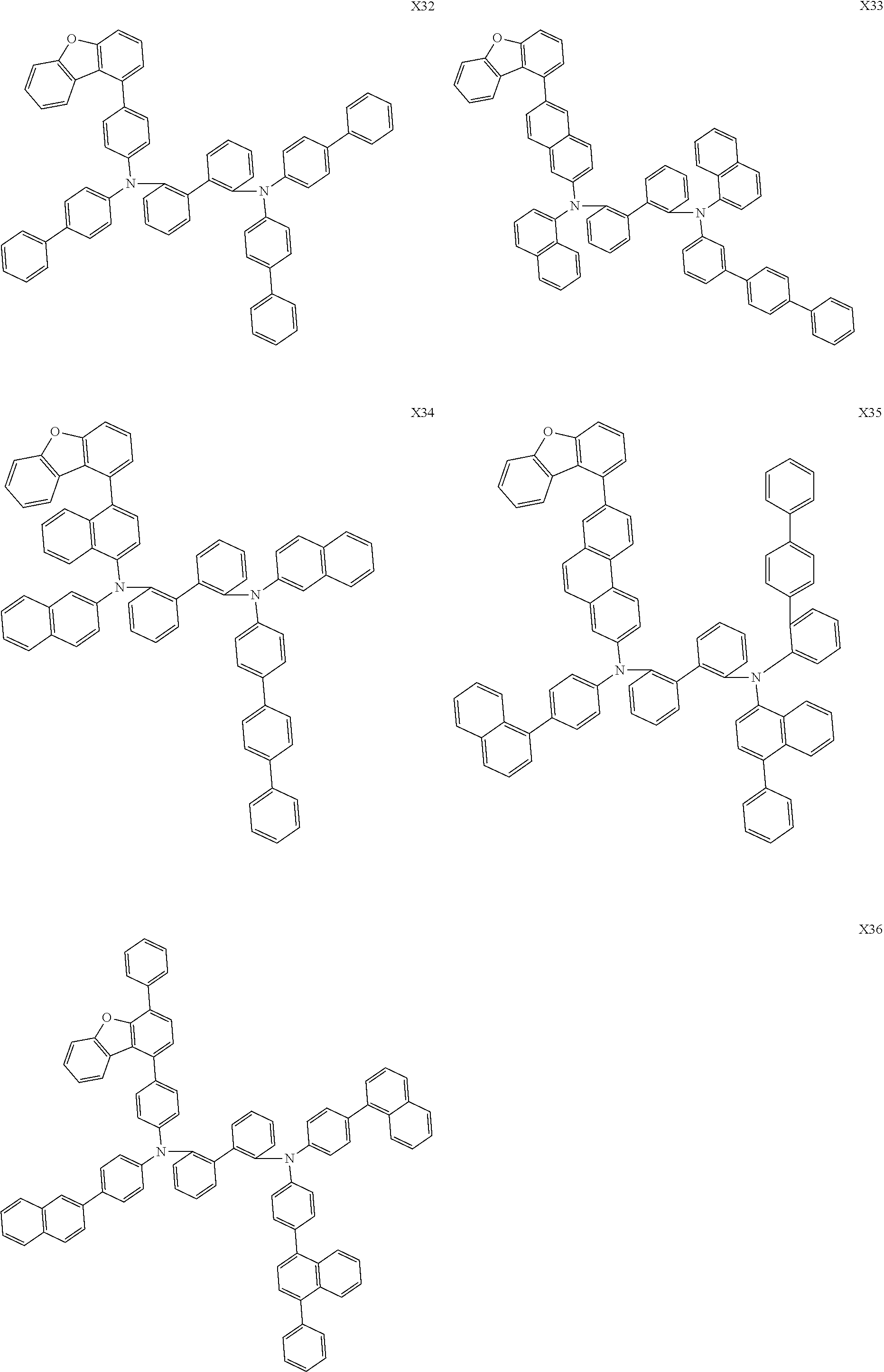

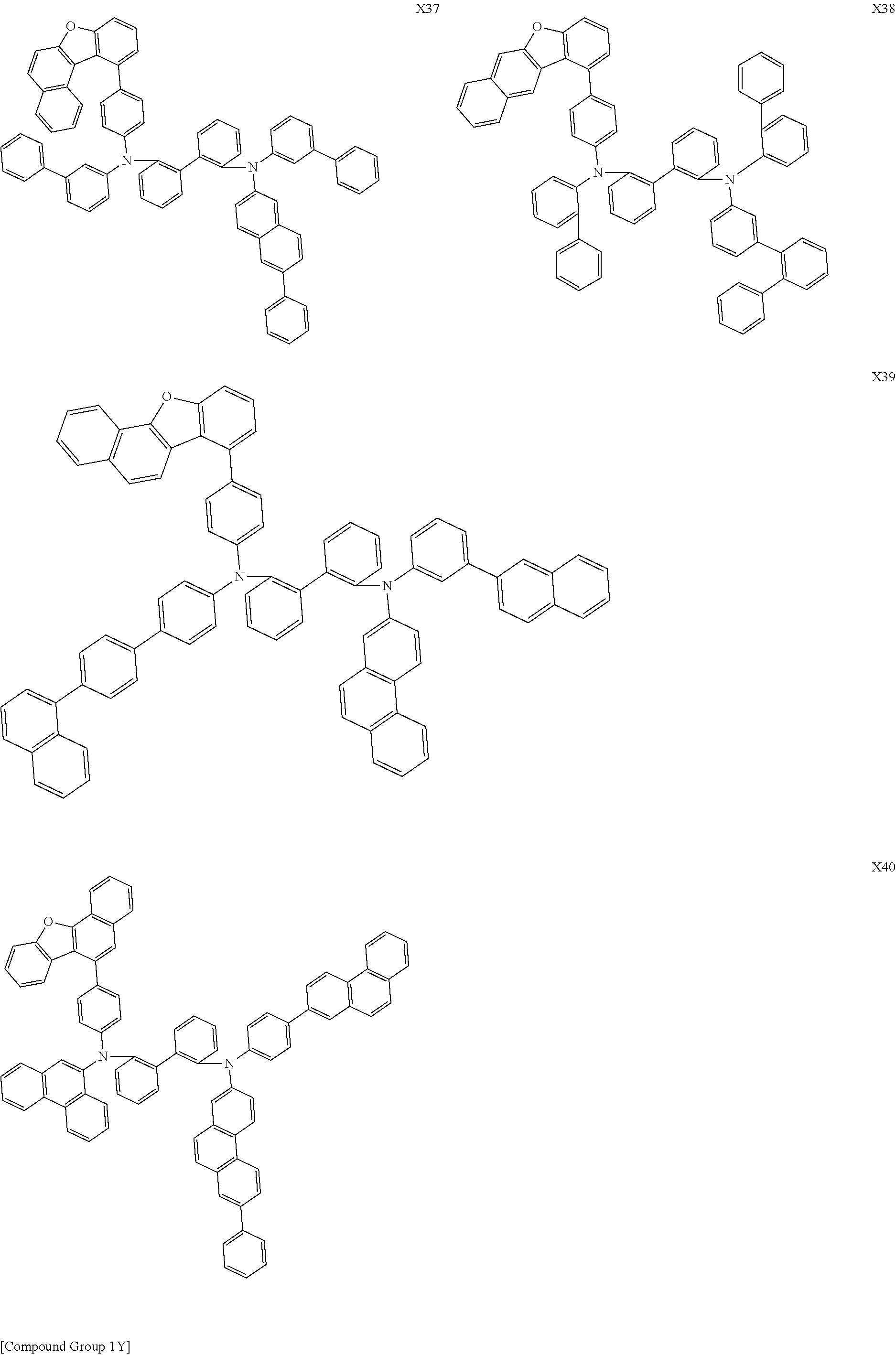

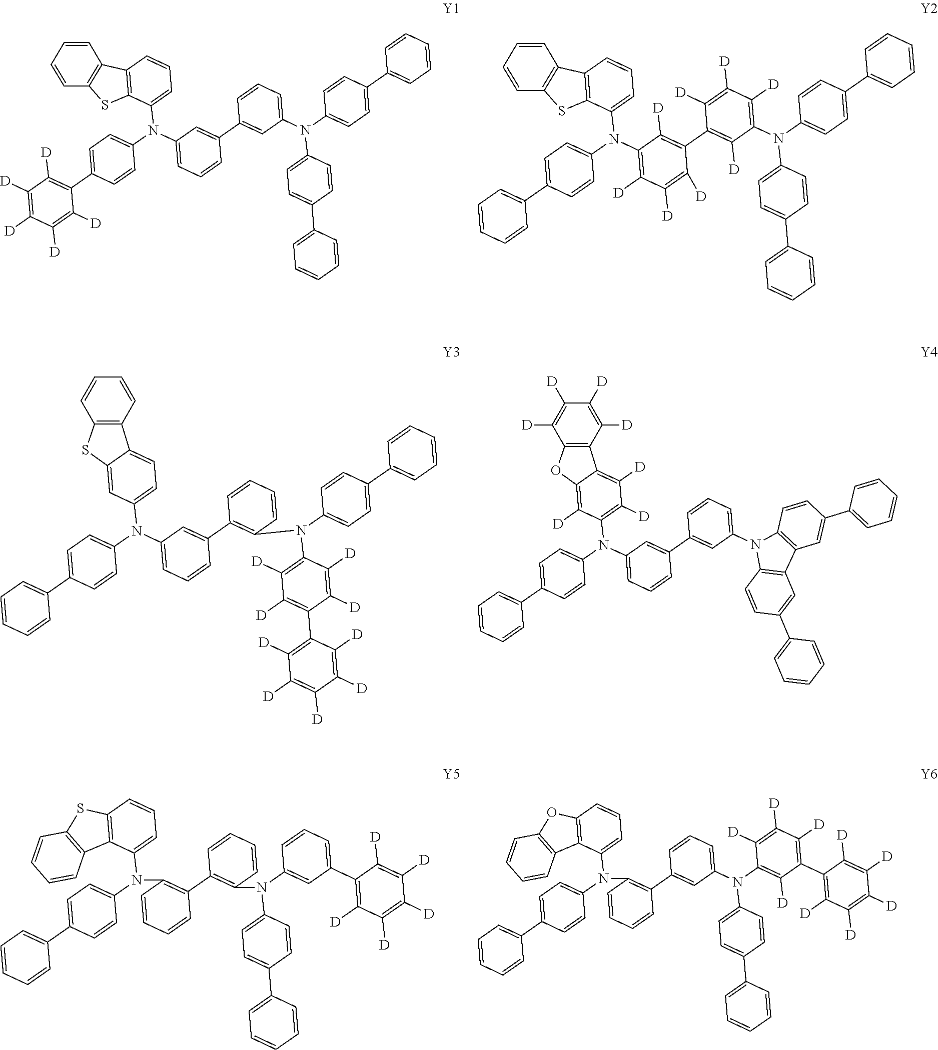

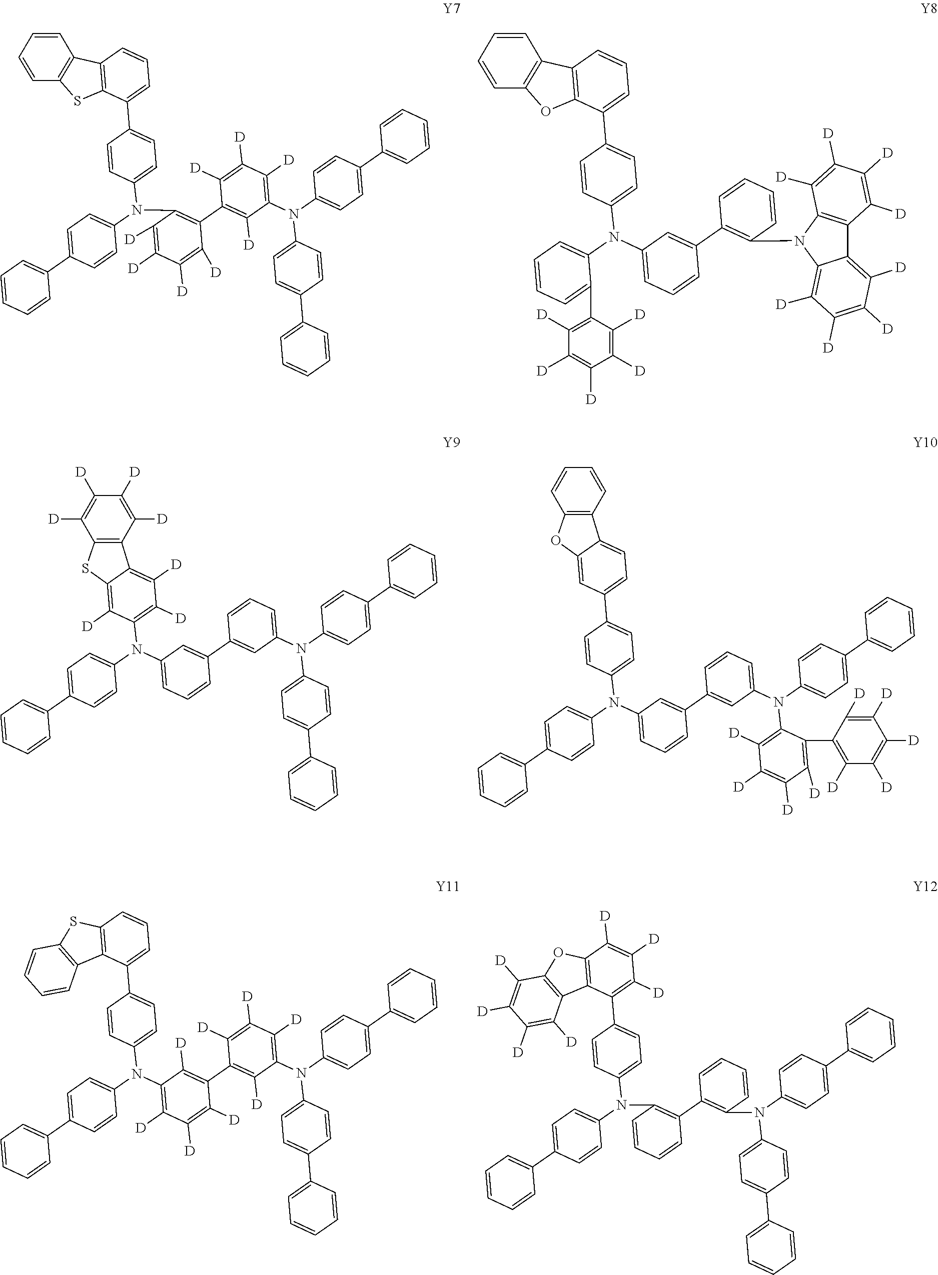

The amine compound according to an embodiment, represented by Formula 1, may include a biphenyl group as a linking moiety and may have a structure in which amine groups are respectively bonded to the benzene rings of the biphenyl group, wherein at least one of the amine groups has a molecular structure including a dibenzoheterole group, thereby showing excellent hole transport capacity and excellent material stability. Accordingly, the light emitting diode according to an embodiment, including the amine compound of an embodiment may show improved emission efficiency and excellent life characteristics.

For example, if the amine compound of an embodiment is used in a hole transport region, hole transport capacity may be improved, recombination probability of holes and electrons in an emission layer may be improved, and emission efficiency may be improved. As described above, by including the amine compound of an embodiment, including a biphenyl group as a linking moiety and having excellent stability, as a material for a light emitting diode, the life of the light emitting diode of an embodiment may be improved.

The light emitting diode ED of an embodiment may further include a material for a hole transport region, which will be explained later, in addition to the above-described amine compound of an embodiment.

The hole transport region HTR may include a compound represented by Formula H-1 below.

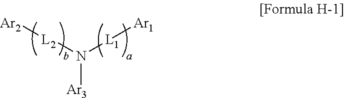

In Formula H-1 above, L₁ and L₂ may each independently be a direct linkage, a substituted or unsubstituted arylene group of 6 to 30 ring-forming carbon atoms, or a substituted or unsubstituted heteroarylene group of 2 to 30 ring-forming carbon atoms. In Formula H-1, a and b may each independently be an integer from 0 to 10. If a or b is 2 or more, multiple L₁ groups or multiple L₂ groups may each independently be a substituted or unsubstituted arylene group of 6 to 30 ring-forming carbon atoms, or a substituted or unsubstituted heteroarylene group of 2 to 30 ring-forming carbon atoms.

In Formula H-1, Ar₁ and Ar₂ may each independently be a substituted or unsubstituted aryl group of 6 to 30 ring-forming carbon atoms, or a substituted or unsubstituted heteroaryl group of 2 to 30 ring-forming carbon atoms. In Formula H-1, Ar₃ may be a substituted or unsubstituted aryl group of 6 to 30 ring-forming carbon atoms, or a substituted or unsubstituted heteroaryl group of 2 to 30 ring-forming carbon atoms.

The compound represented by Formula H-1 may be a monoamine compound. In another embodiment, the compound represented by Formula H-1 may be a diamine compound in which at least one among Ar₁ to Ar₃ includes an amine group as a substituent. The compound represented by Formula H-1 may be a carbazole-based compound in which at least one among Ar₁ and Ar₂ includes a substituted or unsubstituted carbazole group, or a fluorene-based compound in which at least one among Ar₁ and Ar₂ includes a substituted or unsubstituted fluorene group.

The compound represented by Formula H-1 may be represented by any one among the compounds in Compound Group H below. However, the compounds shown in Compound Group H are only examples, and the compound represented by Formula H-1 is not limited to the compounds represented in Compound Group H below.

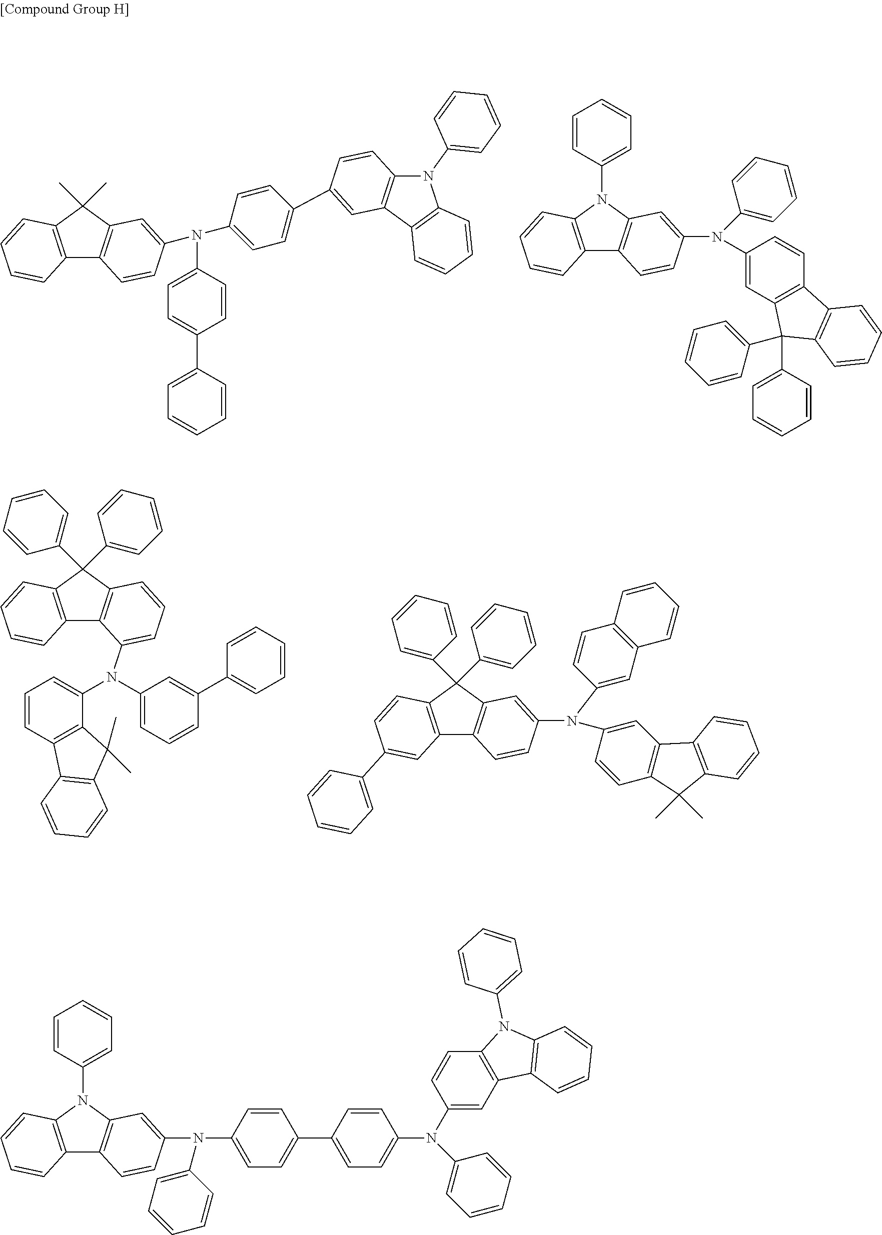 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21 ; c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1]

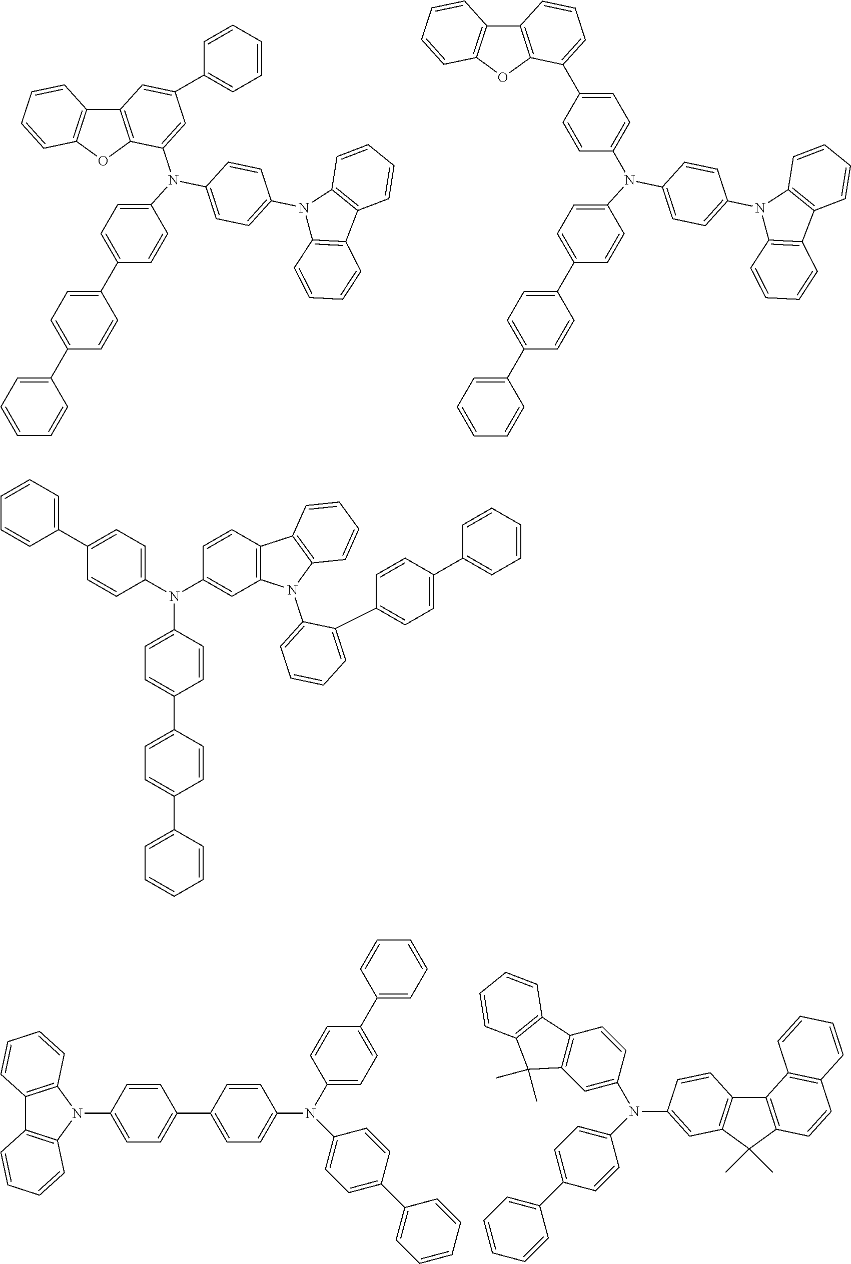

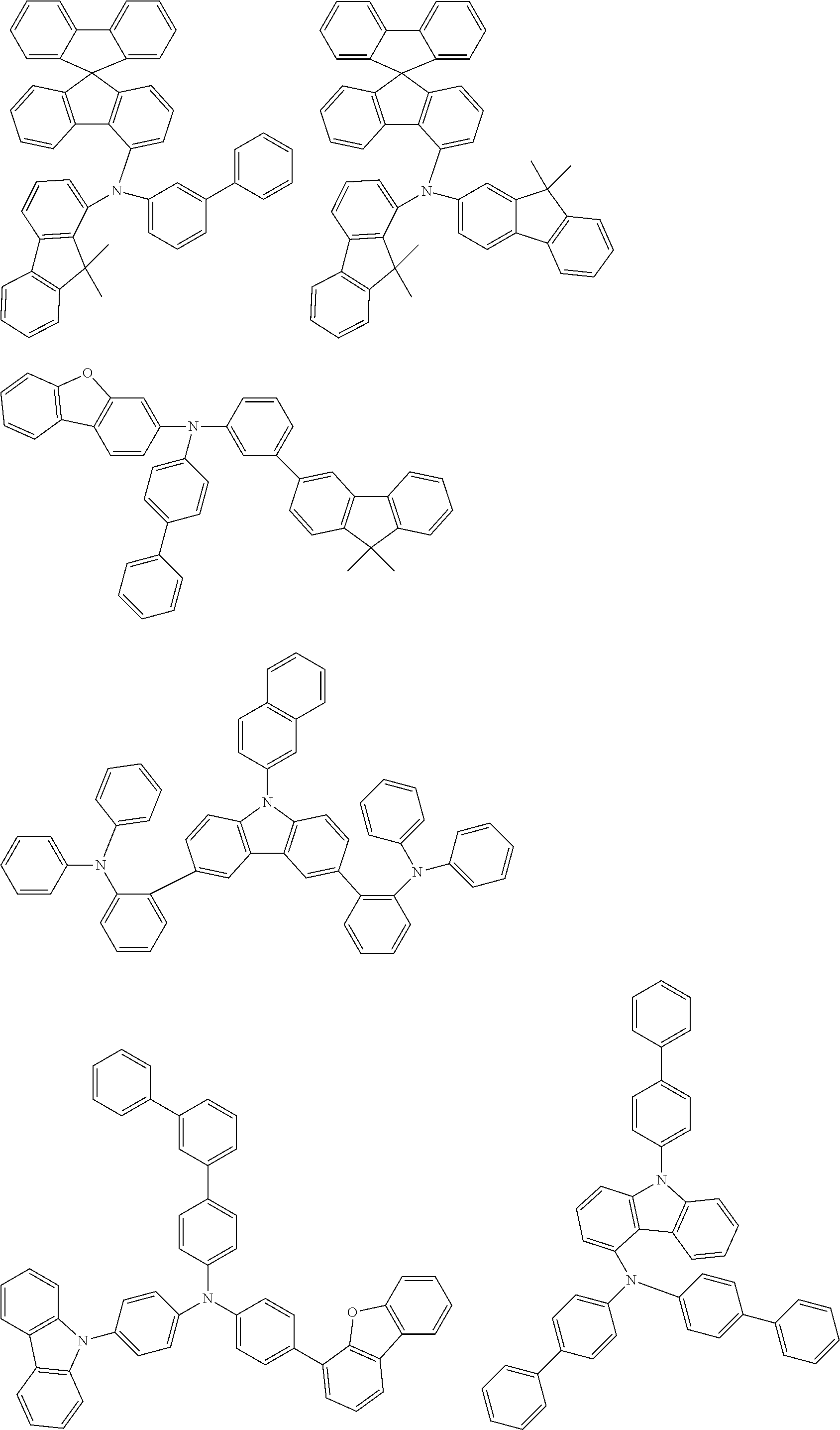

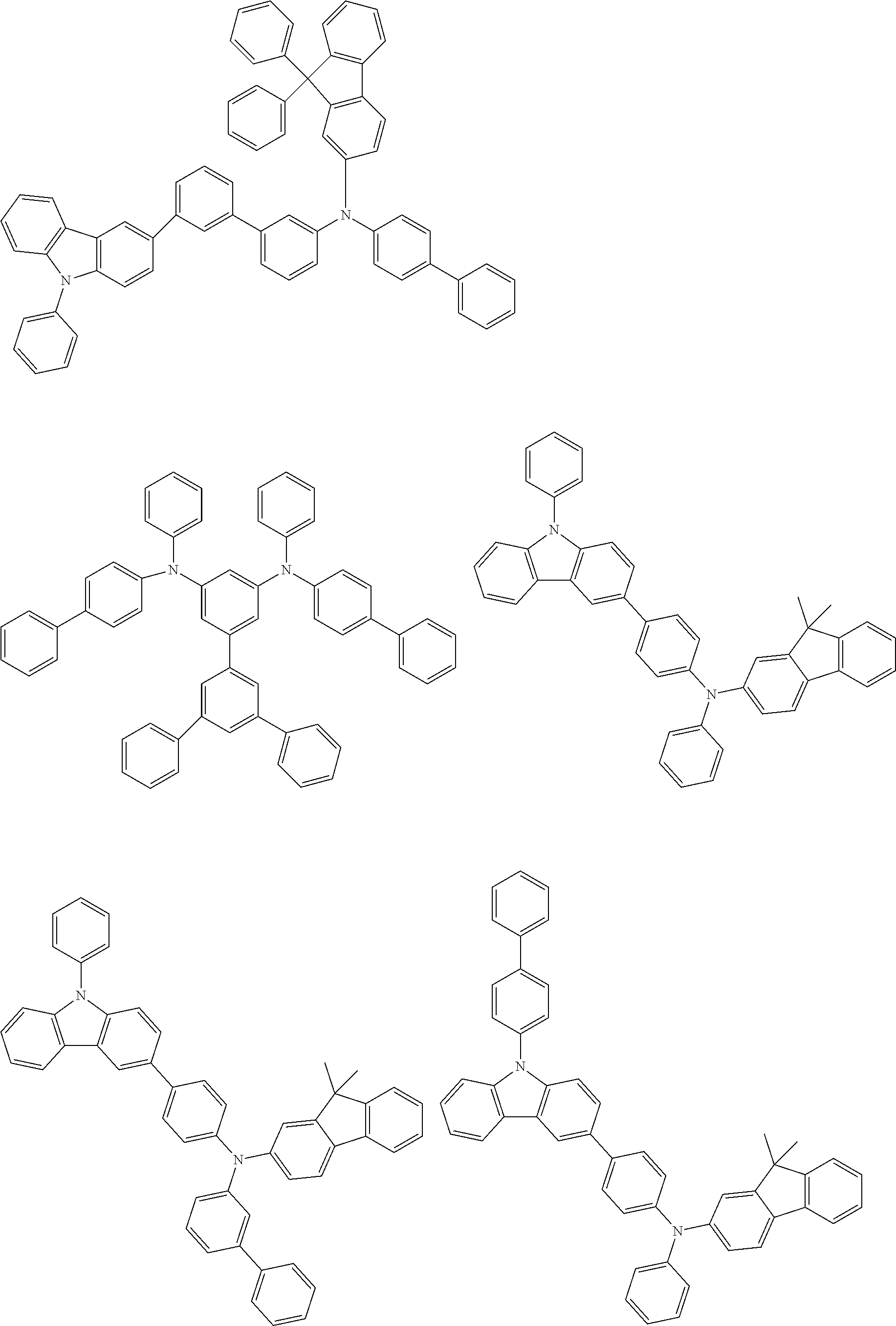

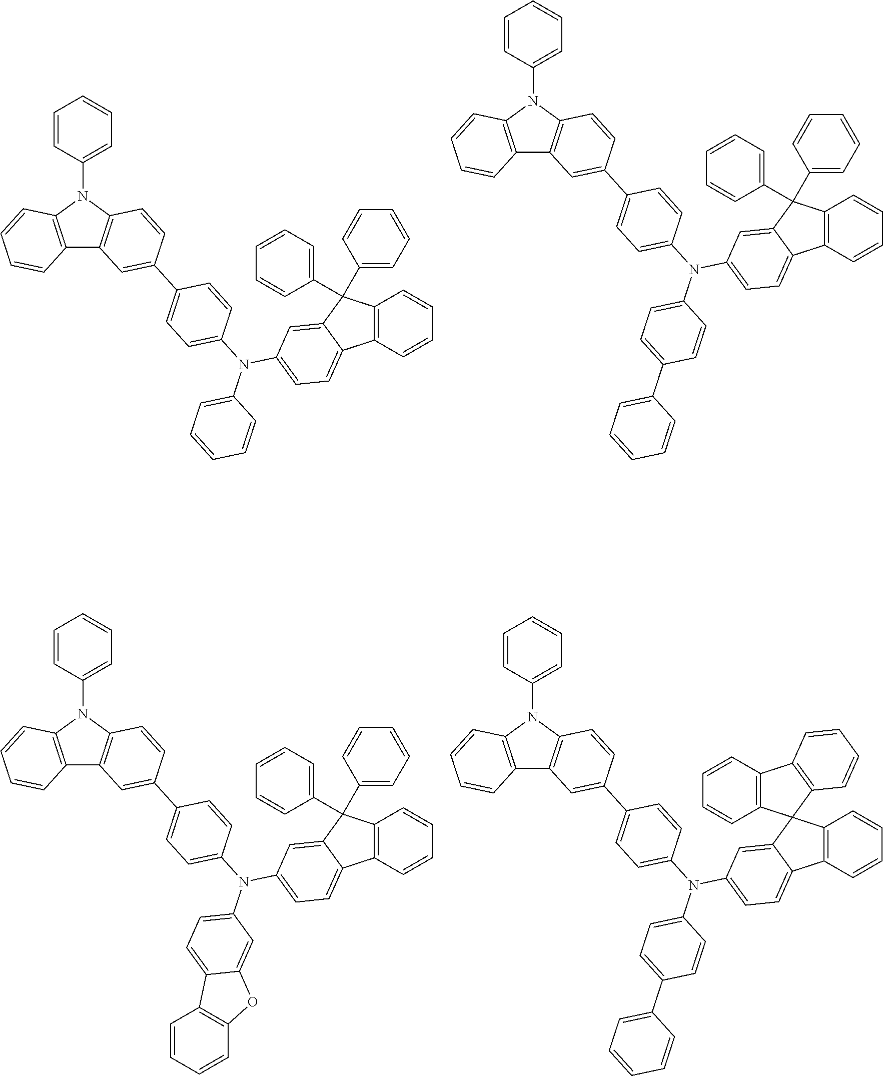

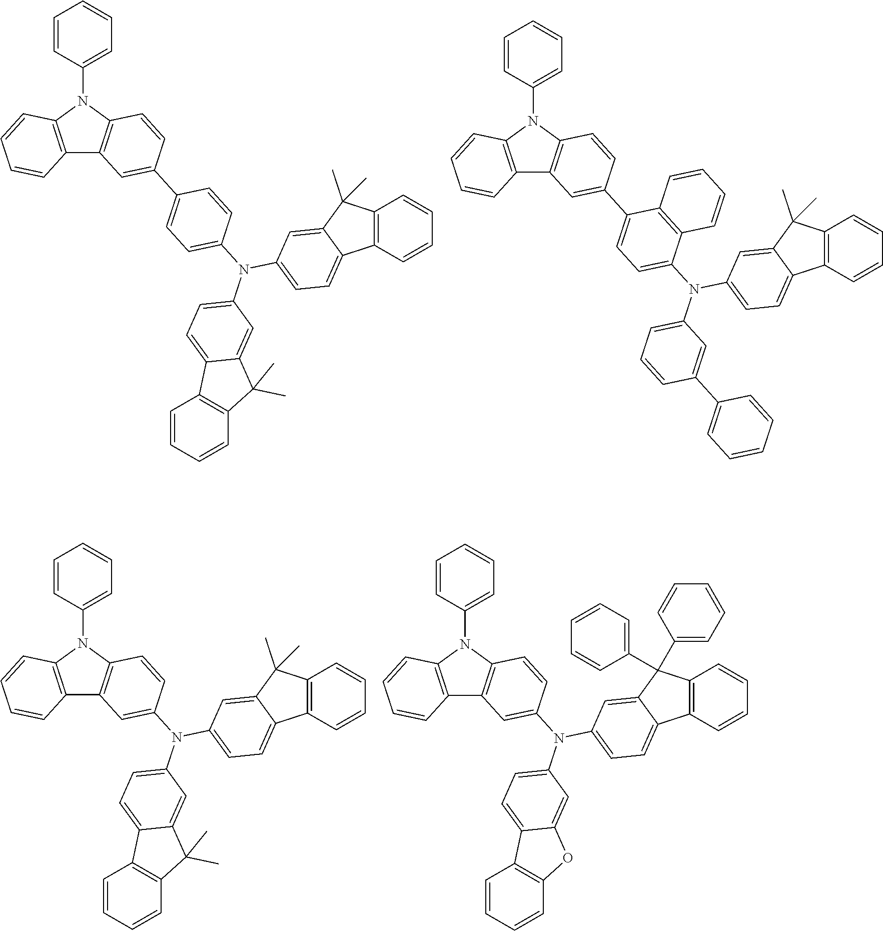

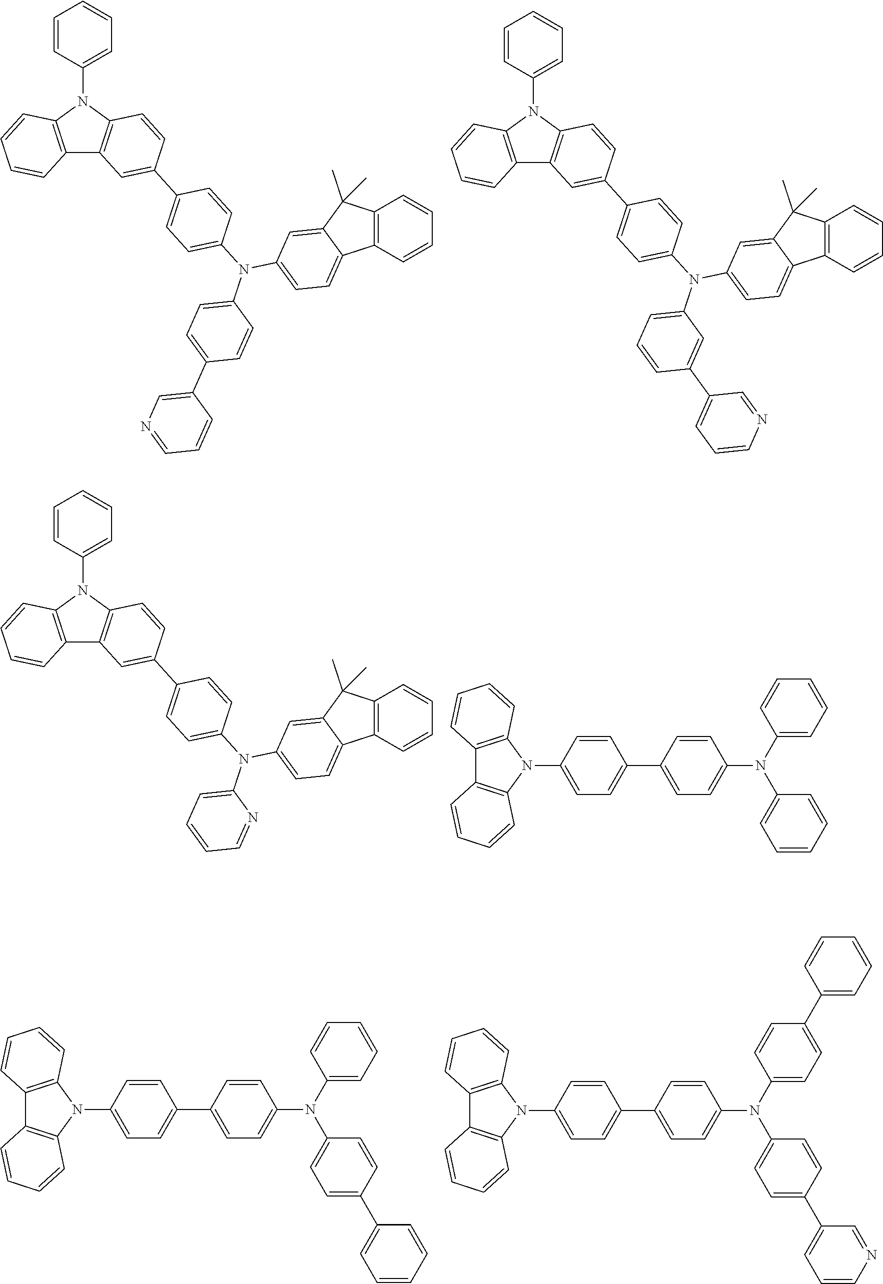

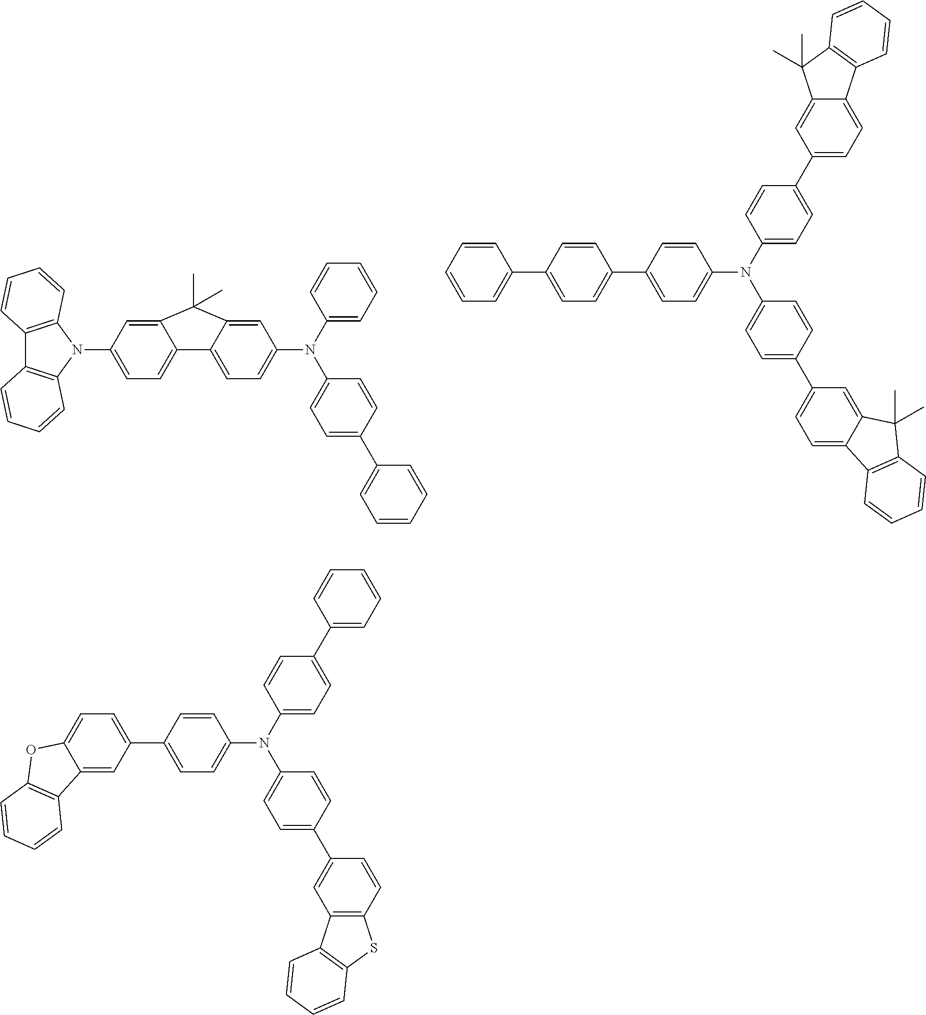 [SMILES: CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21 ; c1ccc(-c2ccc(N(c3ccc(-c4ccc5oc6ccccc6c5c4)cc3)c3ccc(-c4ccc5sc6ccccc6c5c4)cc3)cc2)cc1]

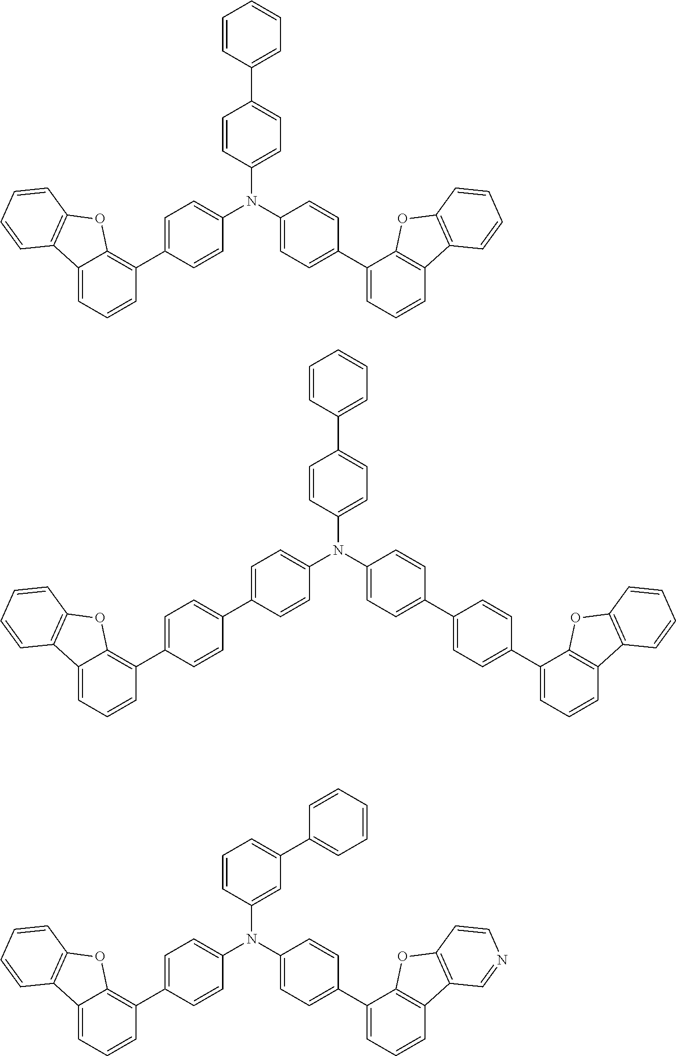 [SMILES: c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1 ; c1ccc(-c2cccc(N(c3ccc(-c4cccc5c4oc4ccccc45)cc3)c3ccc(-c4cccc5c4oc4ccncc45)cc3)c2)cc1]

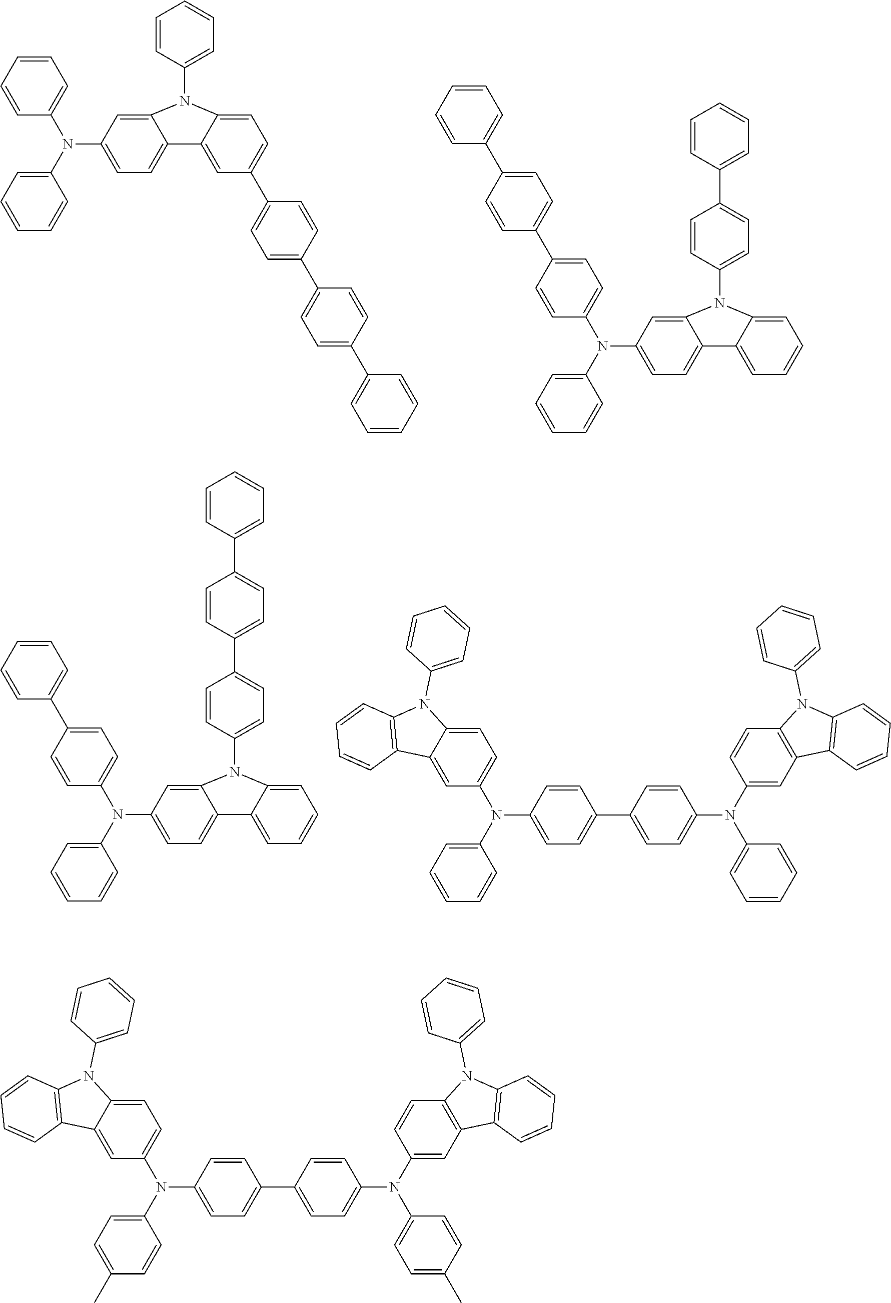 [SMILES: c1ccc(-c2ccc(-c3ccc(-c4ccc5c(c4)c4ccc(N(c6ccccc6)c6ccccc6)cc4n5-c4ccccc4)cc3)cc2)cc1 ; c1ccc(-c2ccc(-c3ccc(N(c4ccccc4)c4ccc5c6ccccc6n(-c6ccc(-c7ccccc7)cc6)c5c4)cc3)cc2)cc1 ; c1ccc(-c2ccc(-c3ccc(-n4c5ccccc5c5ccc(N(c6ccccc6)c6ccc(-c7ccccc7)cc6)cc54)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1]

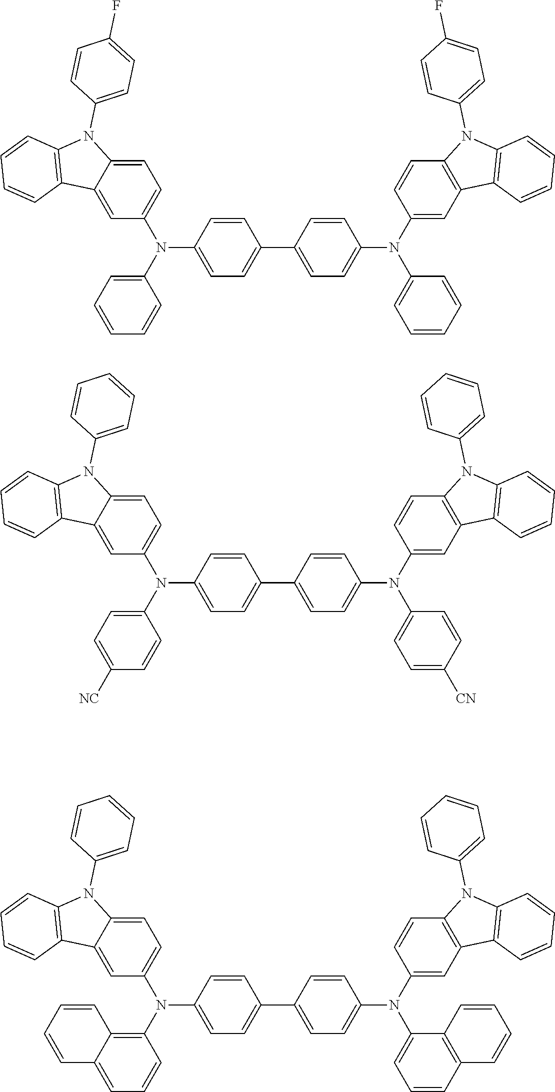

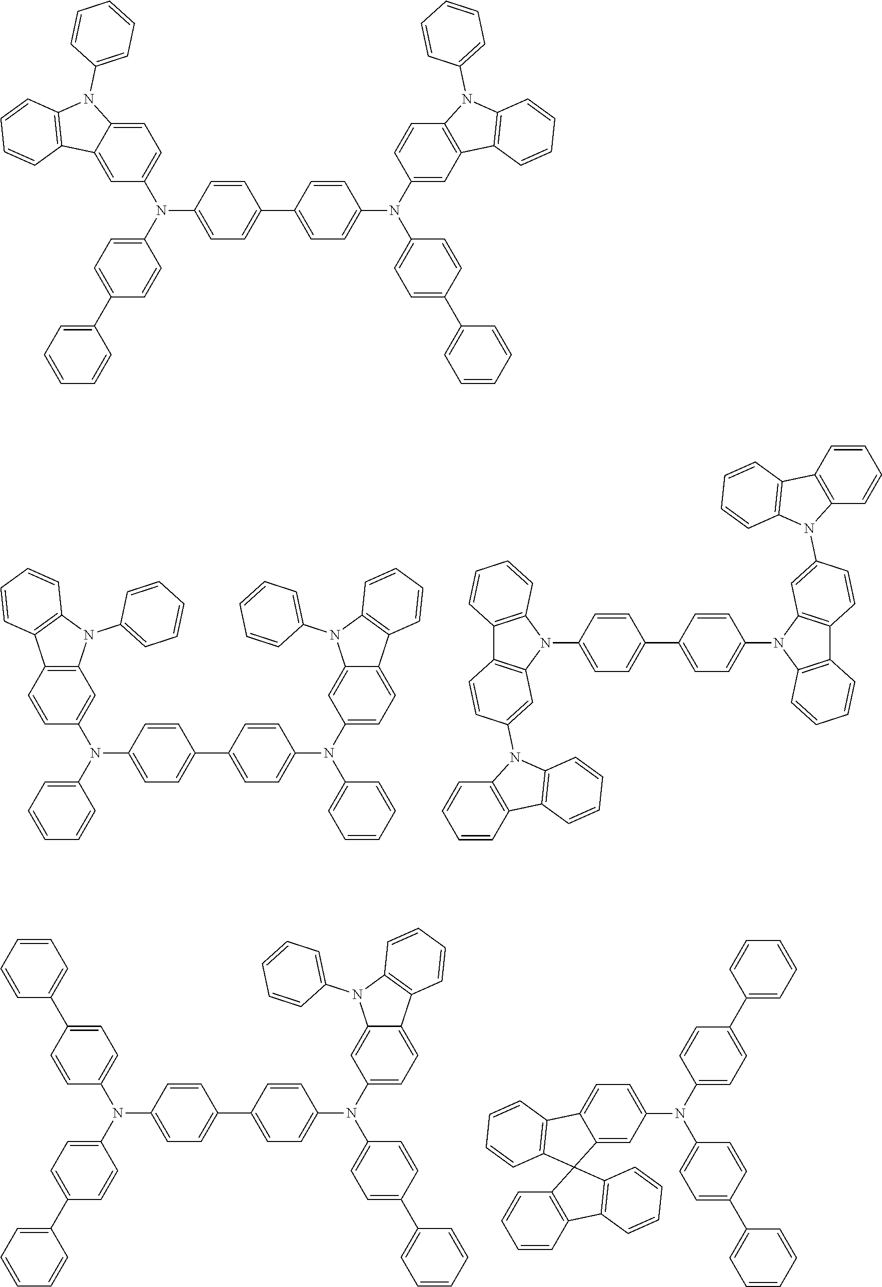

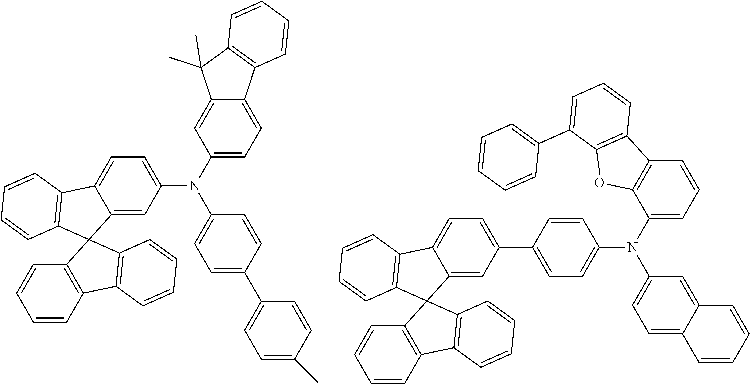

The hole transport region HTR may include a phthalocyanine compound such as copper phthalocyanine, N¹,N^{1′}′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4″,4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(1-naphthalene-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], and dipyrazino[2,3-f:2′,3′-h] quinoxaline-2,3,6,7,10,11-hexacarbonitrile (HAT-CN).

The hole transport region HTR may include carbazole derivatives such as N-phenyl carbazole and polyvinyl carbazole, fluorene-based derivatives, N,N′-bis(3-methylphenyl)-N,N′-diphenyl-[1,1-biphenyl]-4,4′-diamine (TPD), triphenylamine-based derivatives such as 4,4′,4″-tris(N-carbazolyl)triphenylamine (TCTA), N,N′-di(1-naphthalene-1-yl)-N,N′-diphenyl-benzidine (NPB), 4,4′-cyclohexylidene bis[N,N-bis(4-methylphenyl)benzeneamine (TAPC), 4,4′-bis[N,N′-(3-tolyl)amino]-3,3′-dimethylbiphenyl (HMTPD), 9-(4-tert-butylphenyl)-3,6-bis(triphenylsilyl)-9H-carbazole (CzSi), 9-phenyl-9H-3,9′-bicarbazole (CCP), 1,3-bis(N-carbazolyl)benzene (mCP), 1,3-bis(1,8-dimethyl-9H-carbazol-9-yl)benzene (mDCP), etc.

The hole transport region HTR may include the compounds of the hole transport region in at least one among the hole injection layer HIL, hole transport layer HTL, and electron blocking layer EBL.

A thickness of the hole transport region HTR may be in a range of about 100 Å to about 10,000 Å. For example, the thickness of the hole transport region HTR may be in a range of about 100 Å to about 5,000 Å. In case where the hole transport region HTR includes a hole injection layer HIL, a thickness of the hole injection region HIL may be, for example, in a range of about 30 Å to about 1,000 Å. In case where the hole transport region HTR includes a hole transport layer HTL, a thickness of the hole transport layer HTL may be in a range of about 30 Å to about 1,000 Å. For example, in case where the hole transport region HTR includes an electron blocking layer EBL, a thickness of the electron blocking layer EBL may be in a range of about 10 Å to about 1,000 Å. If the thicknesses of the hole transport region HTR, the hole injection layer HIL, the hole transport layer HTL, and the electron blocking layer EBL satisfy the above-described ranges, satisfactory hole transport properties may be achieved without a substantial increase of a driving voltage.

The hole transport region HTR may further include a charge generating material to increase conductivity in addition to the above-described materials. The charge generating material may be dispersed uniformly or non-uniformly in the hole transport region HTR. The charge generating material may be, for example, a p-dopant. The p-dopant may include at least one of metal halide compounds, quinone derivatives, metal oxides, and cyano group-containing compounds, without limitation. For example, the p-dopant may include metal halide compounds such as CuI and RbI, quinone derivatives such as tetracyanoquinodimethane (TCNQ) and 2,3,5,6-tetrafluoro-7,7′,8,8-tetracyanoquinodimethane (F4-TCNQ), metal oxides such as tungsten oxide and molybdenum oxide, and/or cyano group-containing compounds such as dipyrazino[2,3-f: 2′,3′-h] quinoxaline-2,3,6,7,10,11-hexacarbonitrile (HATCN) and 4-[[2,3-bis[cyano-(4-cyano-2,3,5,6-tetrafluorophenyl)methylidene]cyclopropylidene]-cyanomethyl]-2,3,5,6-tetrafluorobenzonitrile, etc., without limitation.

As described above, the hole transport region HTR may further include at least one of a buffer layer (not shown) and an electron blocking layer EBL, in addition to the hole injection layer HIL and the hole transport layer HTL. The buffer layer (not shown) may compensate an optical resonance distance according to the wavelength of light emitted from an emission layer EML and may increase light emission efficiency. Materials which may be included in a hole transport region HTR may be used as materials included in a buffer layer (not shown). The electron blocking layer EBL may be a layer that prevents electron injection from the electron transport region ETR to the hole transport region HTR.

The emission layer EML is provided on the hole transport region HTR. The emission layer EML may have a thickness in a range of about 100 Å to about 1,000 Å. For example, the emission layer EML may have a thickness in a range of about 100 Å to about 300 Å. The emission layer EML may have a single layer formed using a single material, a single layer formed using different materials, or a multilayer structure having layers formed using different materials.

In the light emitting diode ED of an embodiment, the emission layer EML may include anthracene derivatives, pyrene derivatives, fluoranthene derivatives, chrysene derivatives, dihydrobenzanthracene derivatives, or triphenylene derivatives. For example, the emission layer EML may include anthracene derivatives or pyrene derivatives.

In the light emitting diodes ED of embodiments, shown in FIG. 3 to FIG. 6, the emission layer EML may include a host and a dopant, and the emission layer EML may include a compound represented by Formula E-1 below. The compound represented by Formula E-1 below may be used as a fluorescence host material.

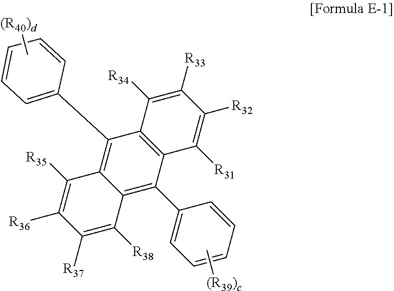

In Formula E-1, R₃₁ to R₄₀ may each independently be a hydrogen atom, a deuterium atom, a halogen atom, a substituted or unsubstituted silyl group, a substituted or unsubstituted alkyl group of 1 to 10 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In Formula E-1, R₃₁ to R₄₀ may be combined with an adjacent group to form a saturated hydrocarbon ring or an unsaturated hydrocarbon ring.

In Formula E-1, c and d may each independently be an integer from 0 to 5.

The compound represented by Formula E-1 may be selected from any one among Compound E1 to Compound E19 below.

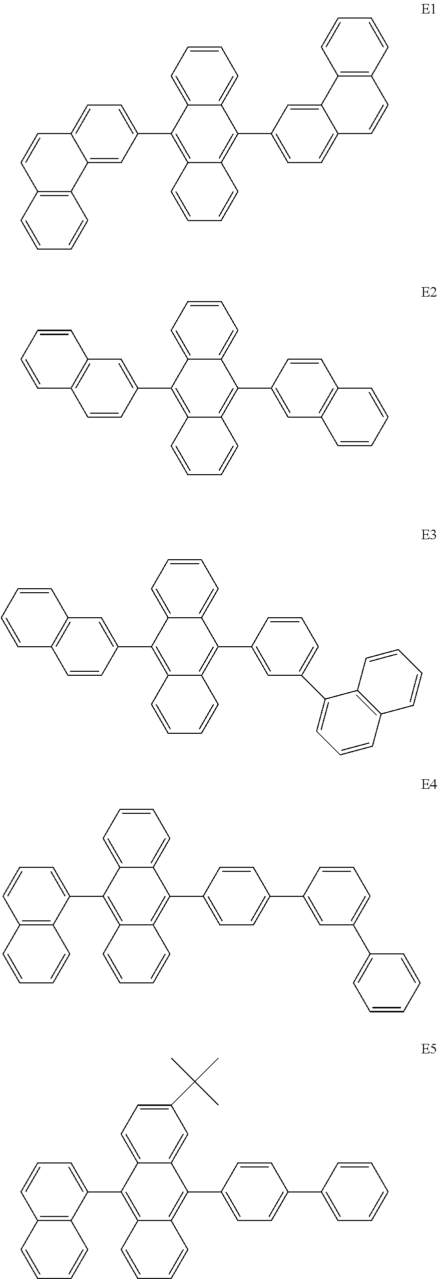

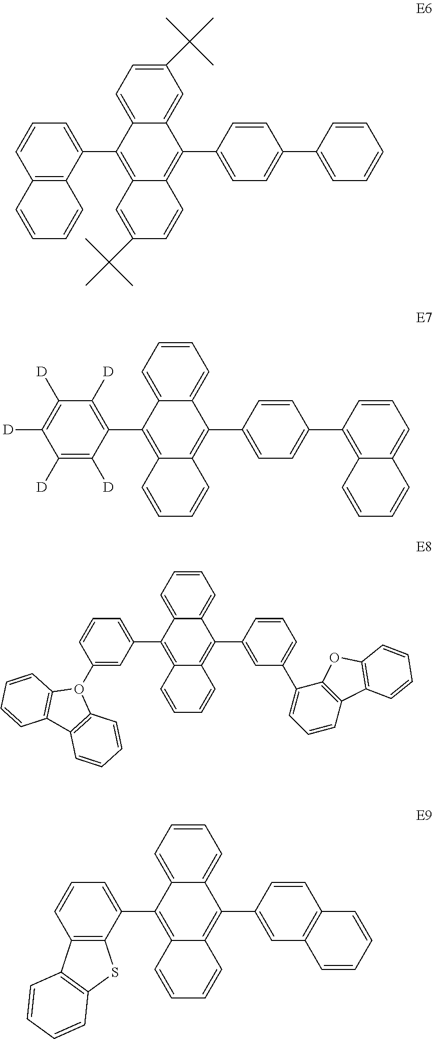

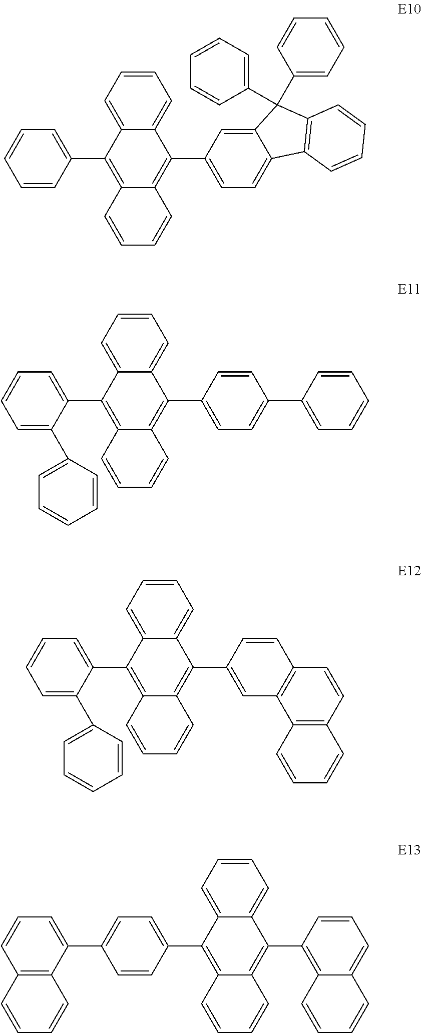

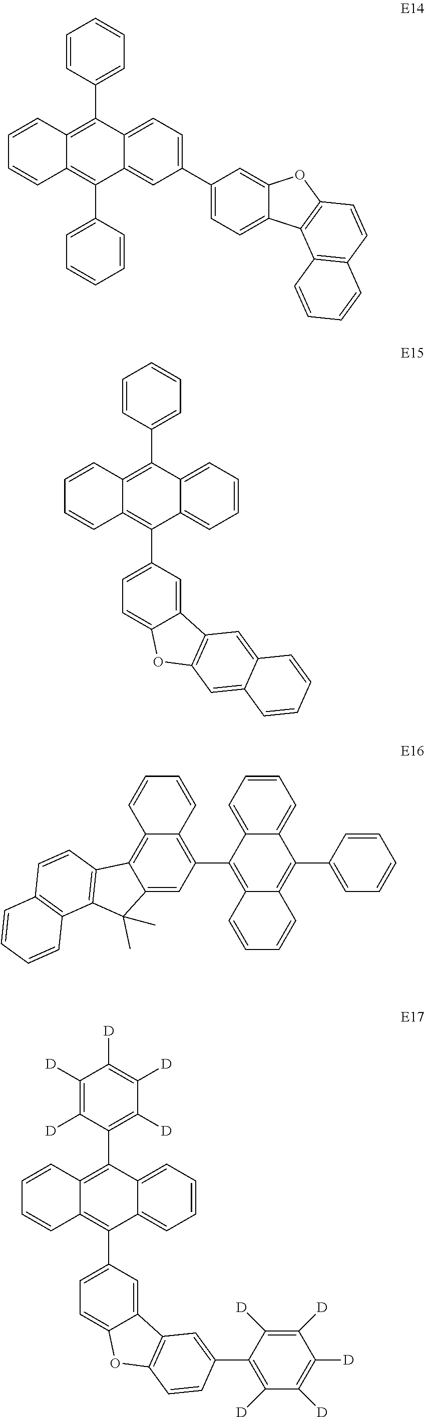

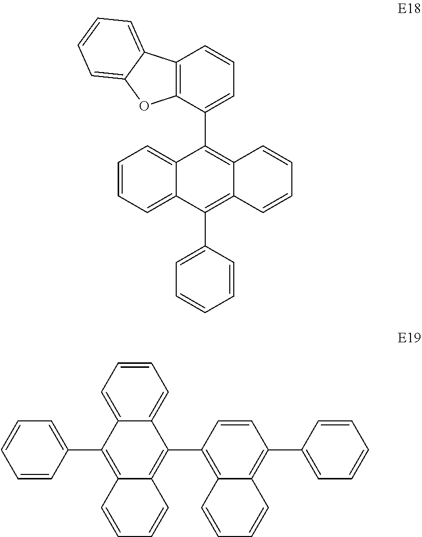

In an embodiment, the compound represented by Formula E-1 may be selected from any one among the compounds below.

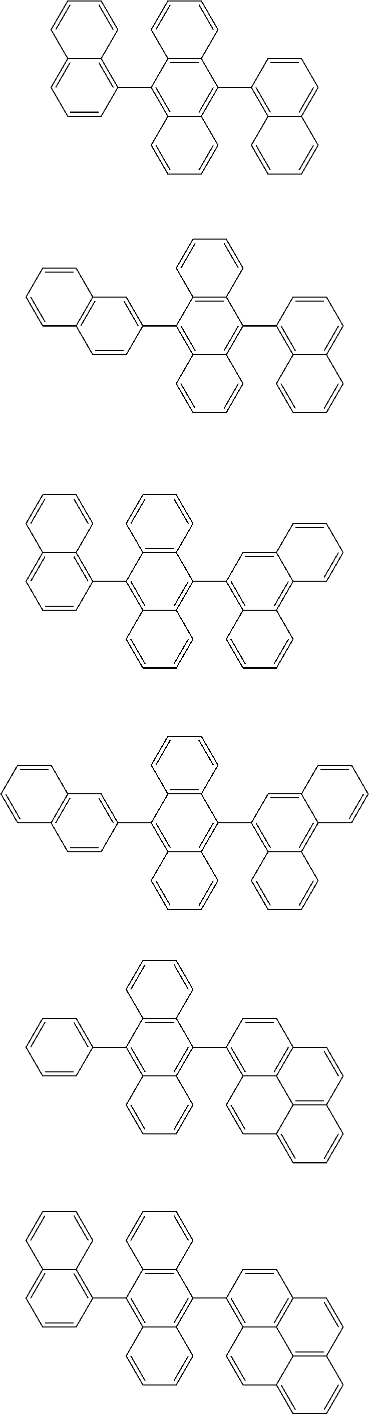

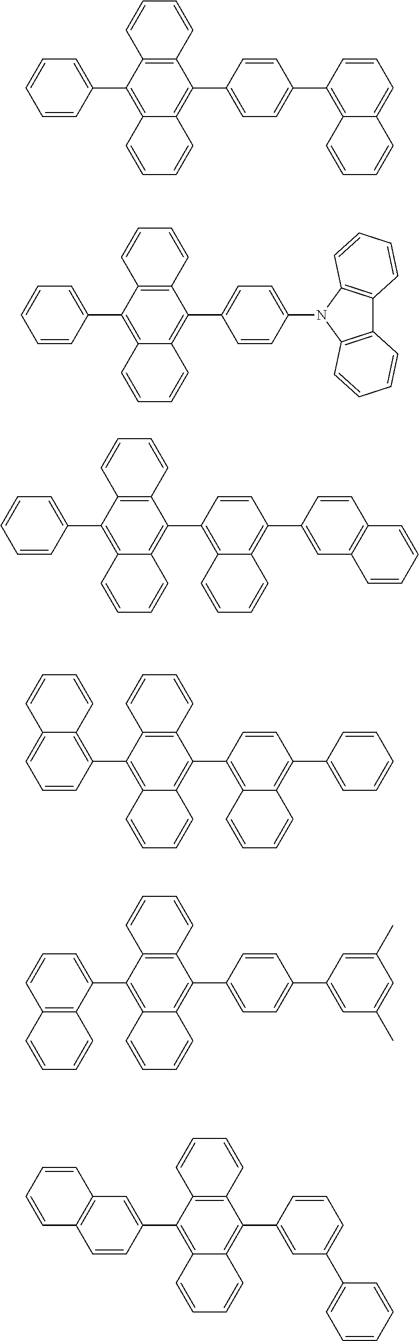

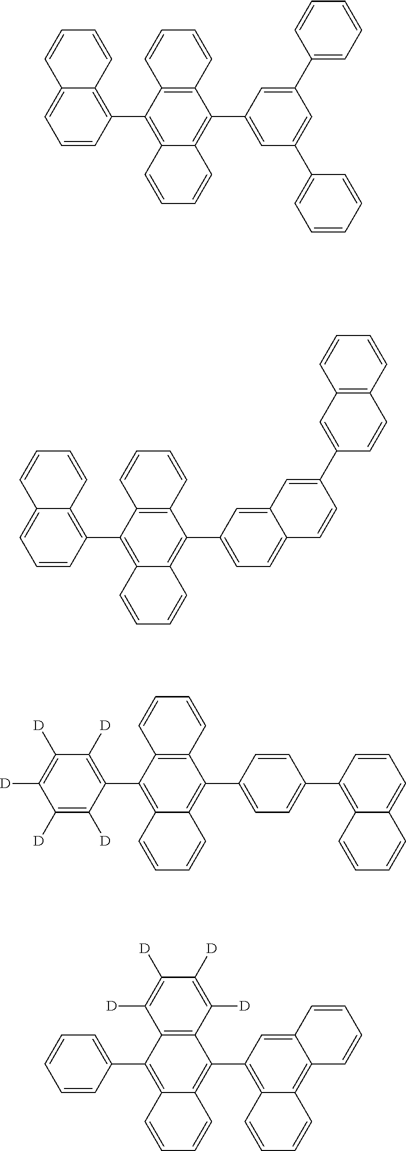

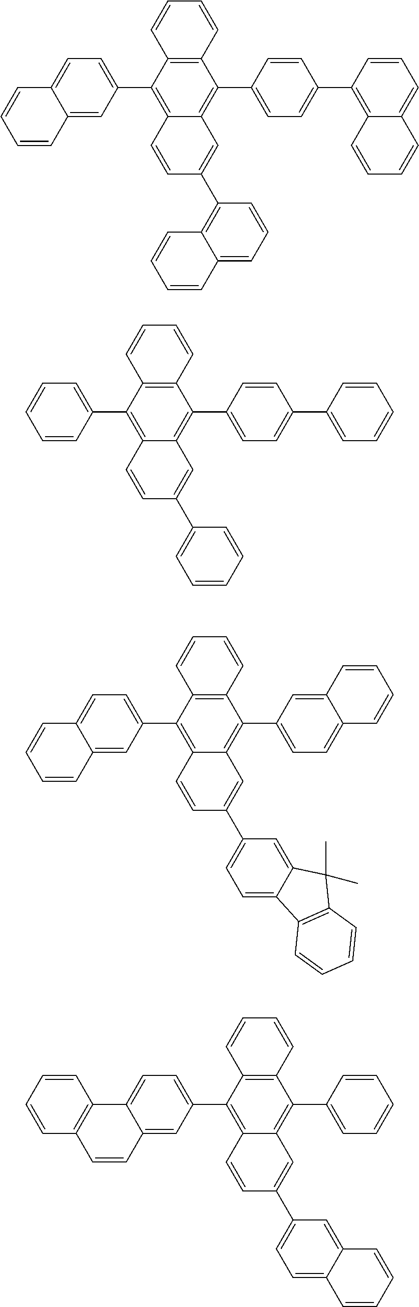

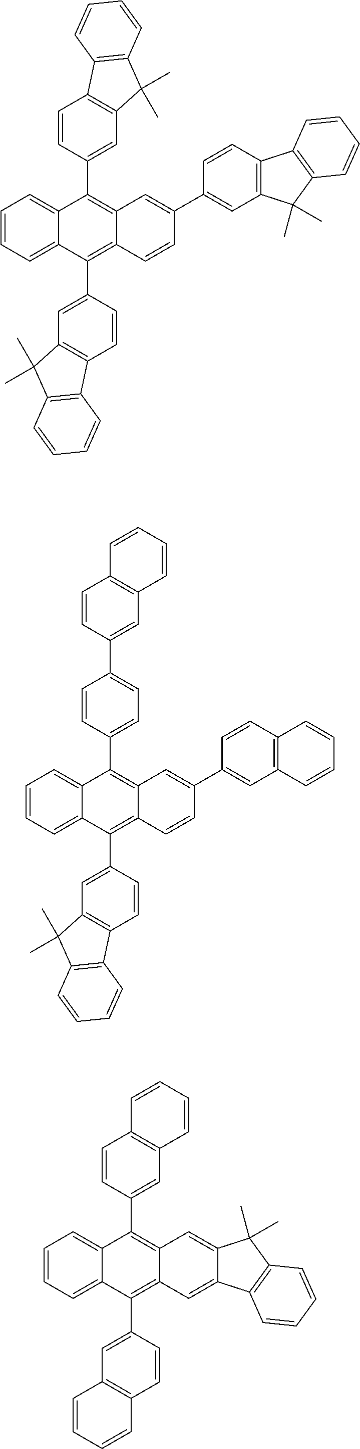

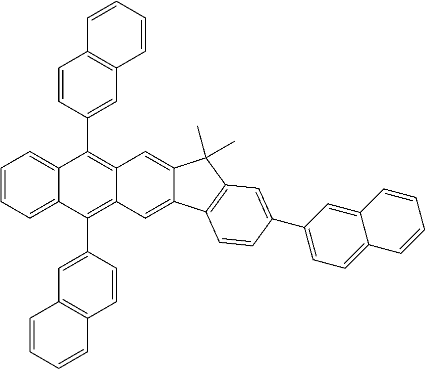 [SMILES: CC1(C)c2cc(-c3ccc4ccccc4c3)ccc2-c2cc3c(-c4ccc5ccccc5c4)c4ccccc4c(-c4ccc5ccccc5c4)c3cc21]

In an embodiment, the emission layer EML may include a compound represented by Formula E-2a or Formula E-2b below. The compound represented by Formula E-2a or Formula E-2b below may be used as a phosphorescence host material.

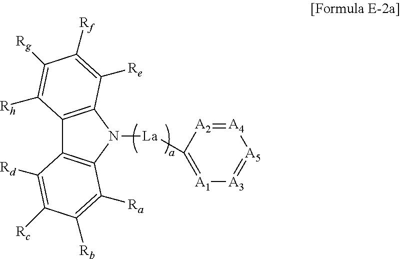

In Formula E-2a, a may be an integer from 0 to 10, and Lₐ may be a direct linkage, a substituted or unsubstituted arylene group of 6 to 30 ring-forming carbon atoms, or a substituted or unsubstituted heteroarylene group of 2 to 30 ring-forming carbon atoms. In Formula E-2a, if a is 2 or more, multiple Lₐ groups may each independently be a substituted or unsubstituted arylene group of 6 to 30 ring-forming carbon atoms, or a substituted or unsubstituted heteroarylene group of 2 to 30 ring-forming carbon atoms.

In Formula E-2a, A₁ to A₅ may each independently be N or C(Ri). Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. Rₐ to Rᵢ may be combined with an adjacent group to form a hydrocarbon ring or a heterocycle including N, O, S, etc. as a ring-forming atom.

In Formula E-2a, two or three of A₁ to A₅ may be N, and the remainder of A₁ to A₅ may be C(Rᵢ).

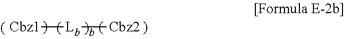

In Formula E-2b, Cbz1 and Cbz2 may each independently be an unsubstituted carbazole group, or a carbazole group substituted with an aryl group of 6 to 30 ring-forming carbon atoms. In Formula E-2b, L_{b} may be a direct linkage, a substituted or unsubstituted arylene group of 6 to 30 ring-forming carbon atoms, or a substituted or unsubstituted heteroarylene group of 2 to 30 ring-forming carbon atoms. In Formula E-2b, b may be an integer from 0 to 10, and if b is 2 or more, multiple L_{b} groups may each independently be a substituted or unsubstituted arylene group of 6 to 30 ring-forming carbon atoms, or a substituted or unsubstituted heteroarylene group of 2 to 30 ring-forming carbon atoms.

The compound represented by Formula E-2a or Formula E-2b may be selected from any one among the compounds in Compound Group E-2 below. However, the compounds shown in Compound Group E-2 below are only examples, and the compound represented by Formula E-2a or Formula E-2b is not limited to the compounds in Compound Group E-2 below.

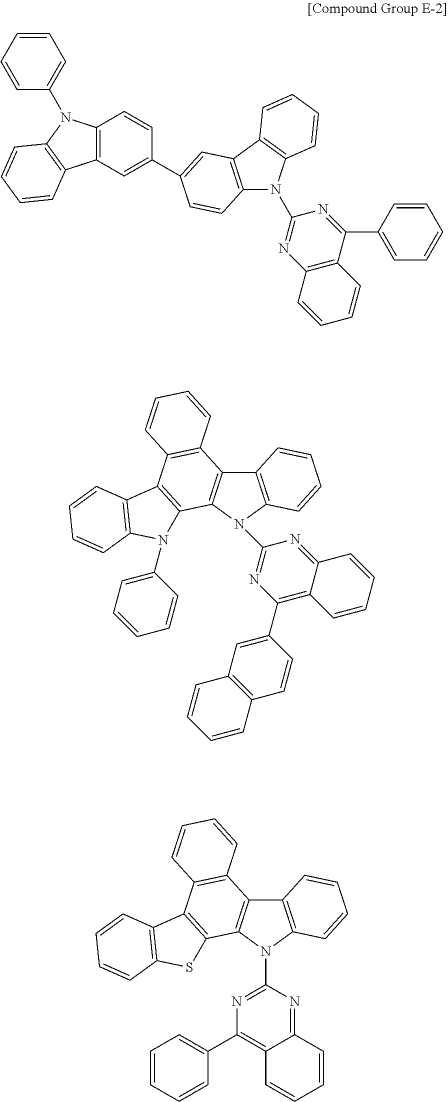 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

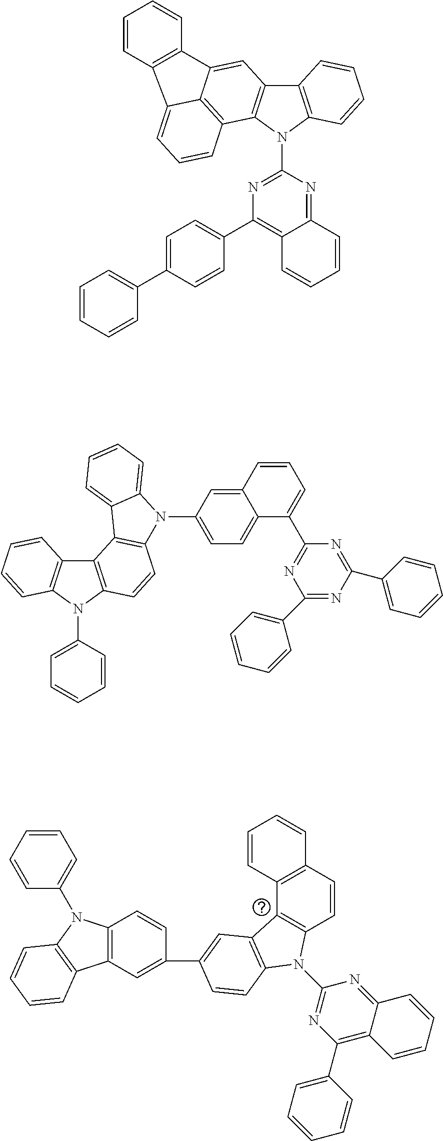 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

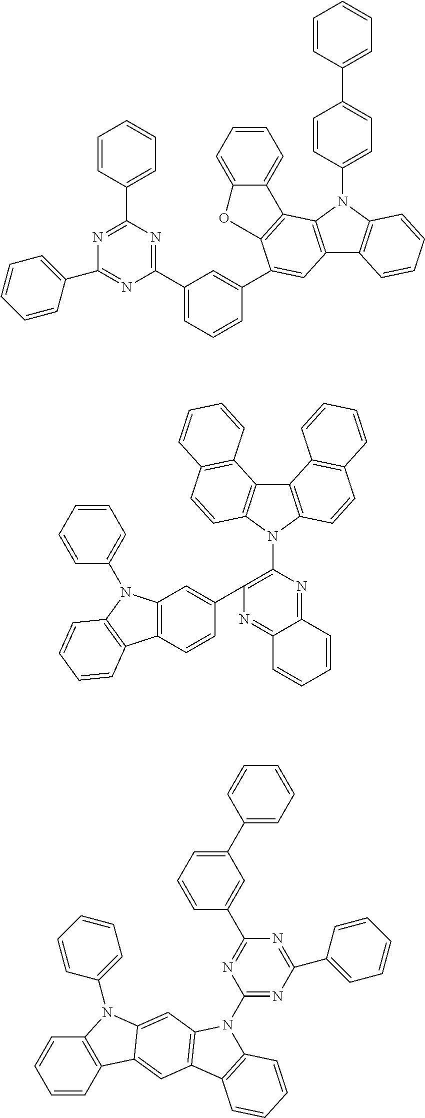 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1]

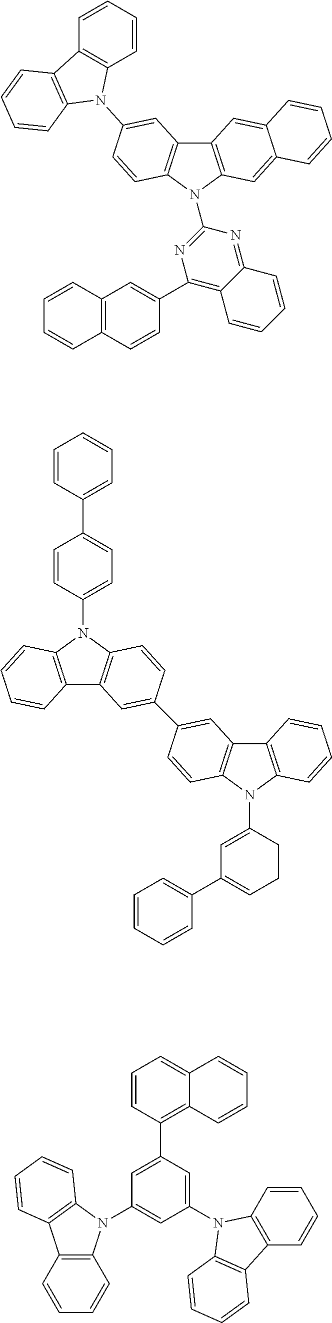 [SMILES: c1ccc2cc(-c3nc(-n4c5ccc(-n6c7ccccc7c7ccccc76)cc5c5cc6ccccc6cc54)nc4ccccc34)ccc2c1 ; C1=C(c2ccccc2)C=C(n2c3ccccc3c3cc(-c4ccc5c(c4)c4ccccc4n5-c4ccc(-c5ccccc5)cc4)ccc32)CC1 ; c1ccc2c(-c3cc(-n4c5ccccc5c5ccccc54)cc(-n4c5ccccc5c5ccccc54)c3)cccc2c1]

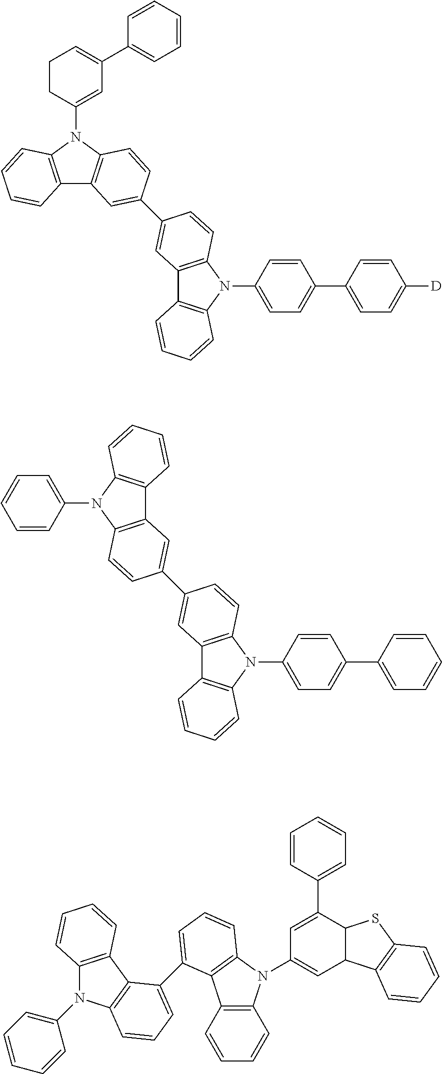 [SMILES: [2H]c1ccc(-c2ccc(-n3c4ccccc4c4cc(-c5ccc6c(c5)c5ccccc5n6C5=CC(c6ccccc6)=CCC5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; C1=C(c2ccccc2)C2Sc3ccccc3C2C=C1n1c2ccccc2c2c(-c3cccc4c3c3ccccc3n4-c3ccccc3)cccc21]

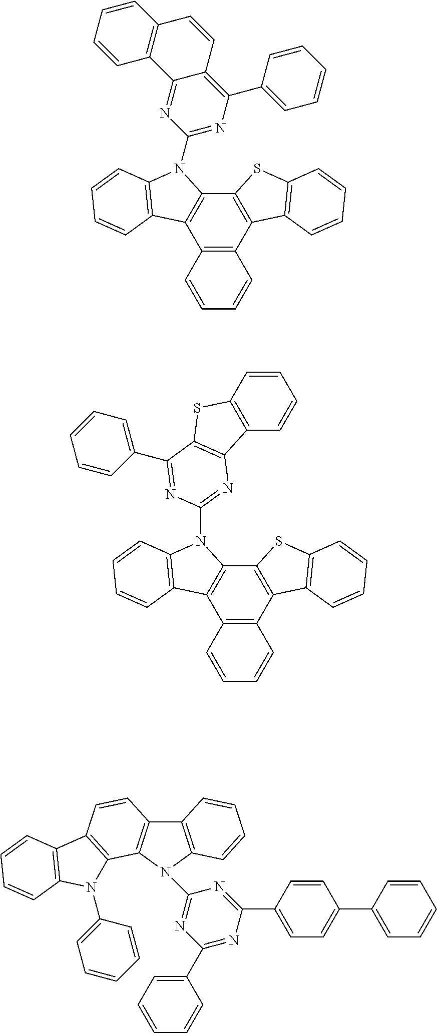 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1]

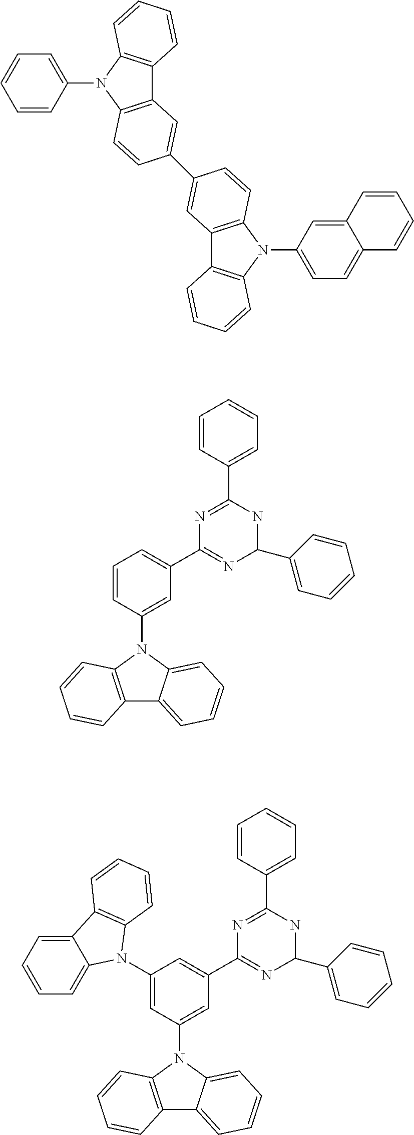 [SMILES: c1ccc(-n2c3ccccc3c3cc(-c4ccc5c(c4)c4ccccc4n5-c4ccc5ccccc5c4)ccc32)cc1 ; c1ccc(C2=NC(c3cccc(-n4c5ccccc5c5ccccc54)c3)=NC(c3ccccc3)N2)cc1 ; c1ccc(C2=NC(c3cc(-n4c5ccccc5c5ccccc54)cc(-n4c5ccccc5c5ccccc54)c3)=NC(c3ccccc3)N2)cc1]

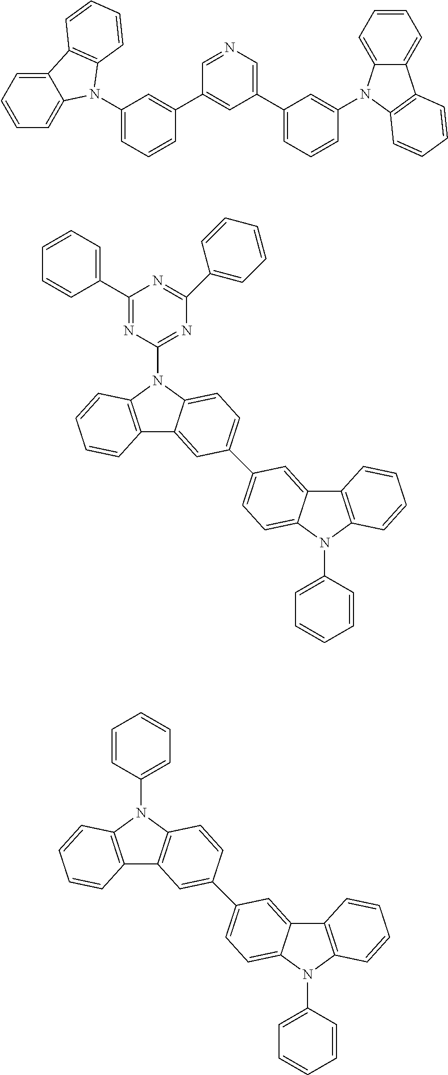 [SMILES: c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-n2c3ccccc3c3cc(-c4ccc5c(c4)c4ccccc4n5-c4ccccc4)ccc32)cc1]

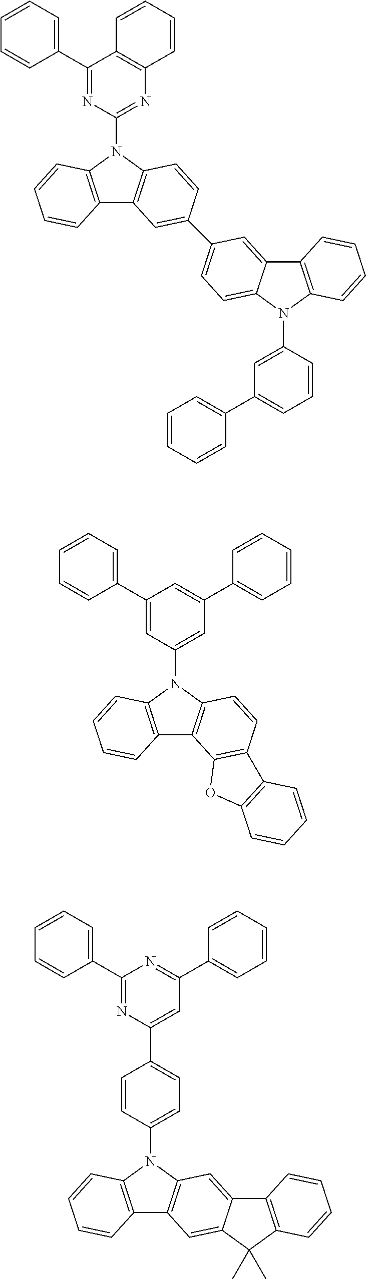 [SMILES: c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1 ; CC1(C)c2ccccc2-c2cc3c(cc21)c1ccccc1n3-c1ccc(-c2cc(-c3ccccc3)nc(-c3ccccc3)n2)cc1]

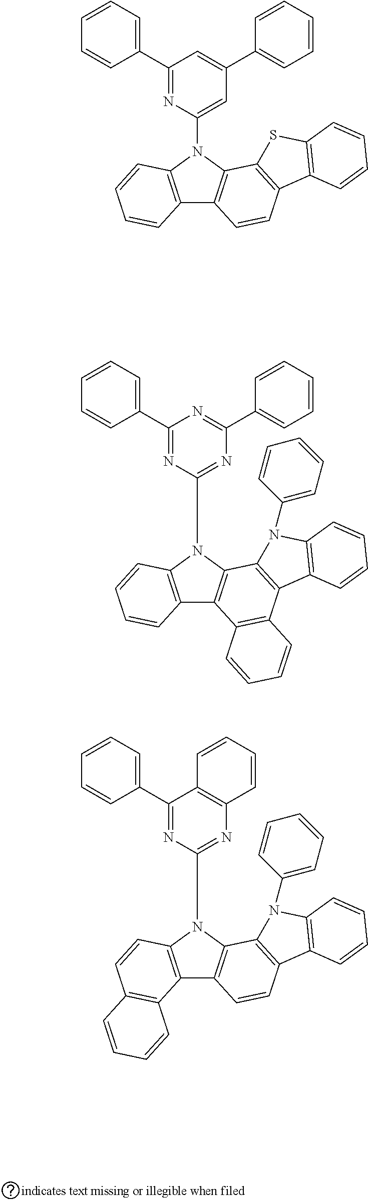 [SMILES: c1ccc(-c2cc(-c3ccccc3)nc(-n3c4ccccc4c4ccc5c6ccccc6sc5c43)c2)cc1 ; c1ccc(-c2nc(-c3ccccc3)nc(-n3c4ccccc4c4c5ccccc5c5c6ccccc6n(-c6ccccc6)c5c43)n2)cc1 ; c1ccc(-c2nc(-n3c4ccc5ccccc5c4c4ccc5c6ccccc6n(-c6ccccc6)c5c43)nc3ccccc23)cc1]

The emission layer EML may further include a common material in the art as a host material. For example, the emission layer EML may include as a host material, at least one of bis[2-(diphenylphosphino)phenyl] ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), and 1,3,5-tris(1-phenyl-1H-benzo[d]imidazole-2-yl)benzene (TPBi). However, embodiments are not limited thereto. For example, tris(8-hydroxyquinolino)aluminum (Alq₃), poly(N-vinylcarbazole) (PVK), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be used as the host material.

The emission layer EML may include a compound represented by Formula M-a or Formula M-b below. The compound represented by Formula M-a or Formula M-b may be used as a phosphorescence dopant material.

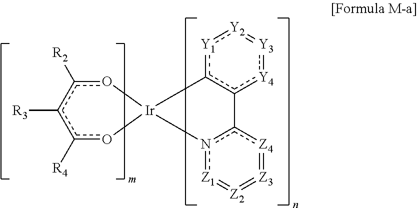

In Formula M-a, Y₁ to Y₄, and Z₁ to Z₄ may each independently be C(R₁) or N, and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In Formula M-a, m may be 0 or 1, and n may be 2 or 3. In Formula M-a, if m is 0, n may be 3, and if m is 1, n may be 2.

The compound represented by Formula M-a may be used as a phosphorescence dopant.

The compound represented by Formula M-a may be selected from any one among Compounds M-a1 to M-a23 below. However, Compounds M-a1 to M-a23 below are examples, and the compound represented by Formula M-a is not limited to the compounds represented by Compounds M-a1 to M-a23 below.

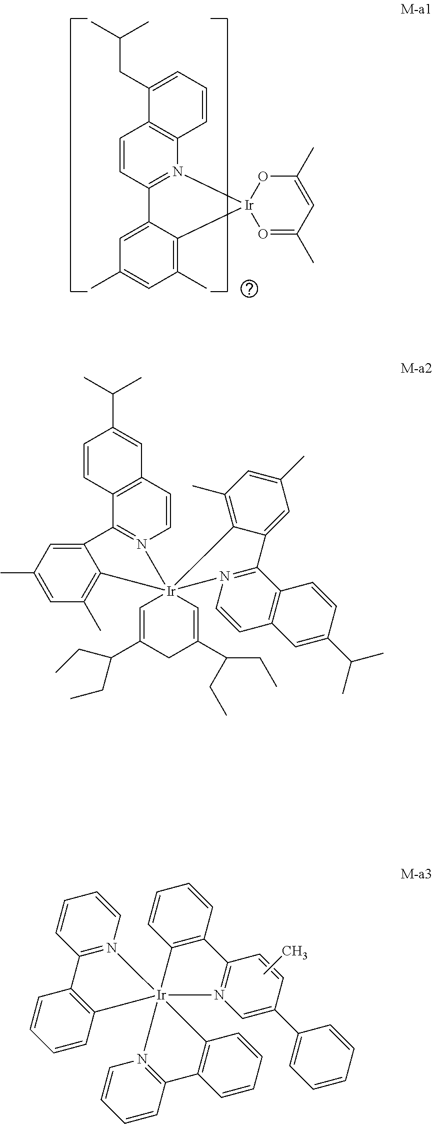

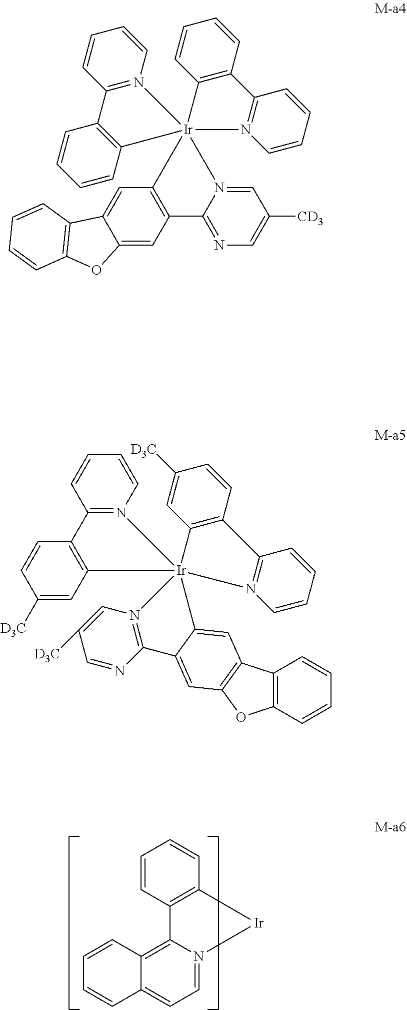

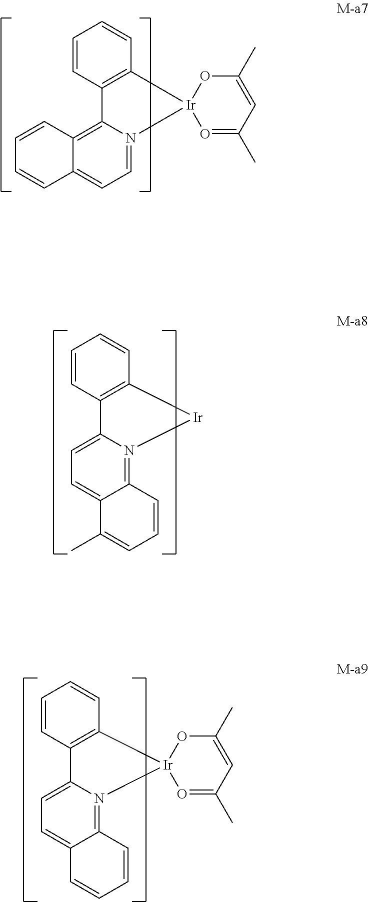

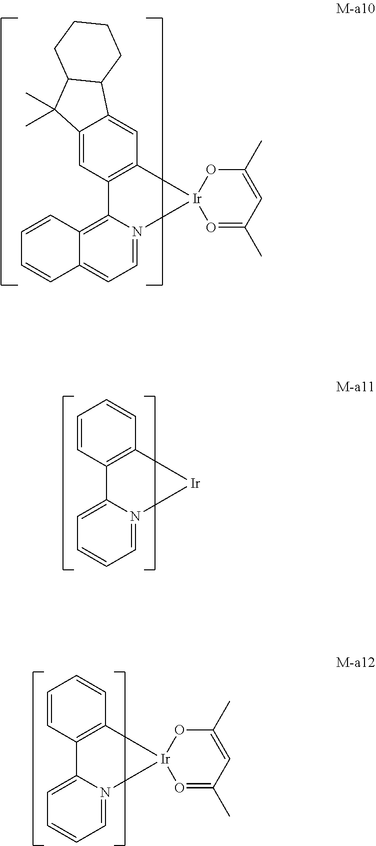

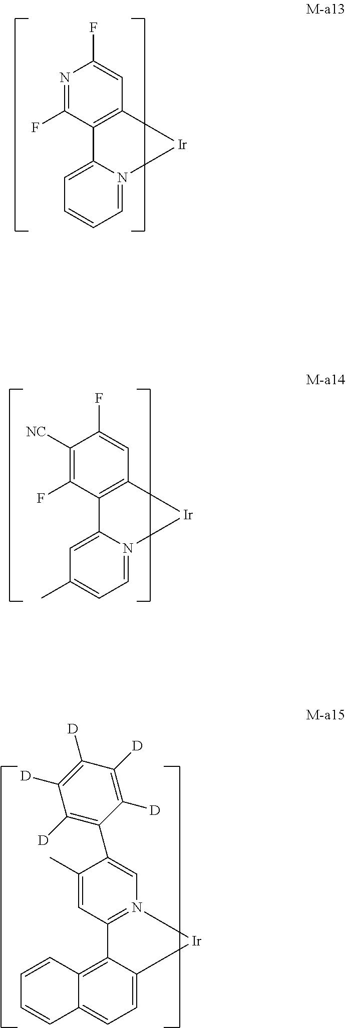

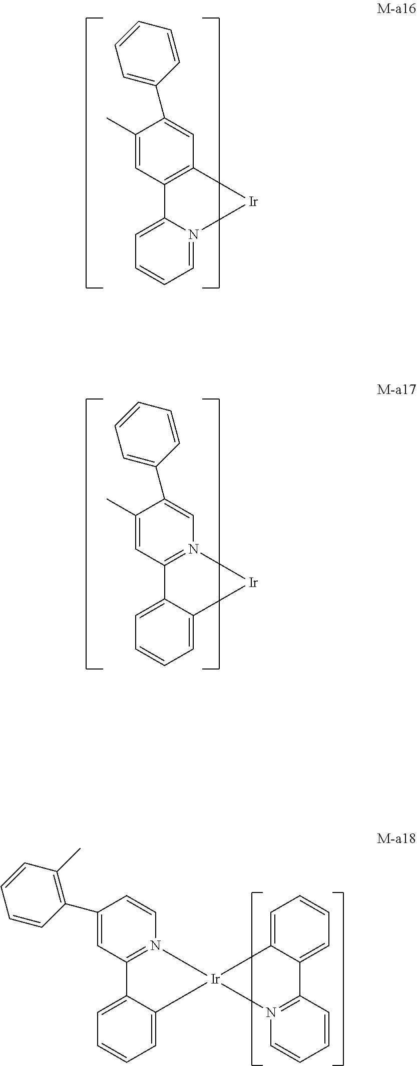

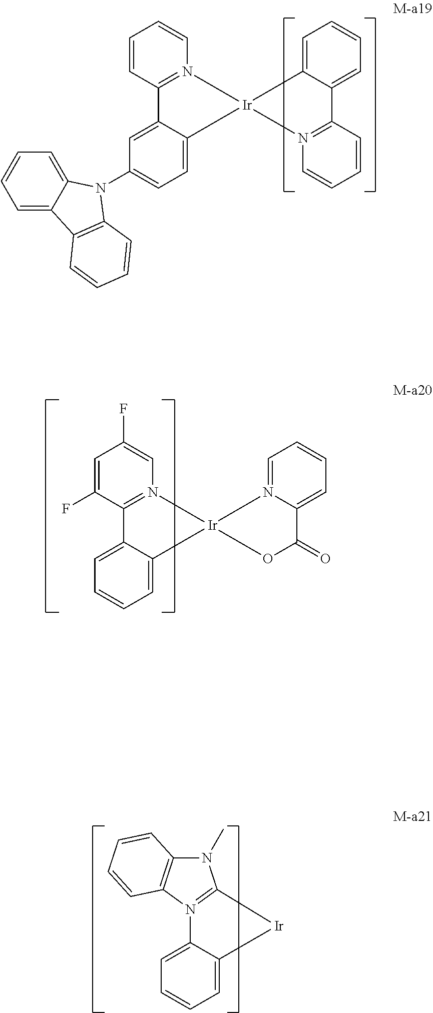

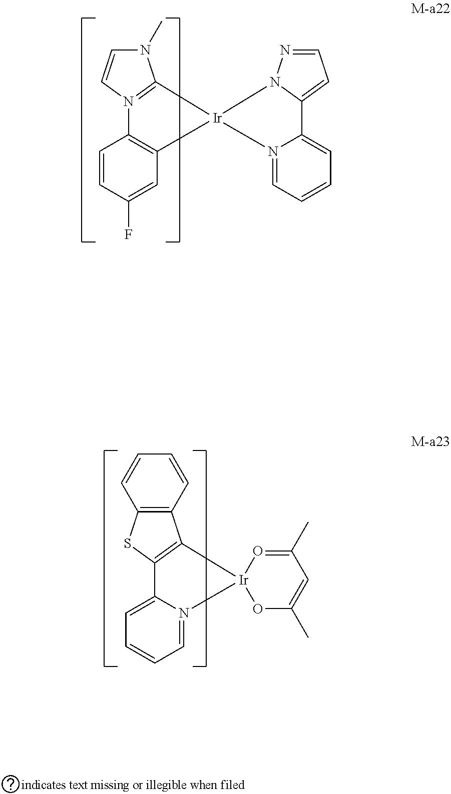

Compound M-a1 and Compound M-a2 may be used as red dopant materials, and Compound M-a3 to Compound M-a5 may be used as green dopant materials.

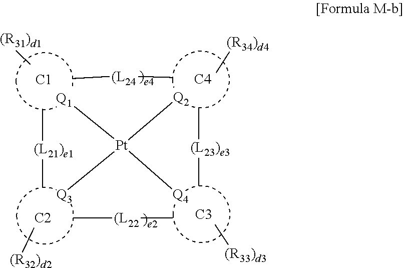

In Formula M-b, Q₁ to Q₄ may each independently be C or N, C1 to C4 may each independently be a substituted or unsubstituted hydrocarbon ring of 5 to 30 ring-forming carbon atoms, or a substituted or unsubstituted heterocycle of 2 to 30 ring-forming carbon atoms. In Formula M-b, L21 to L24 may each independently be a direct linkage,

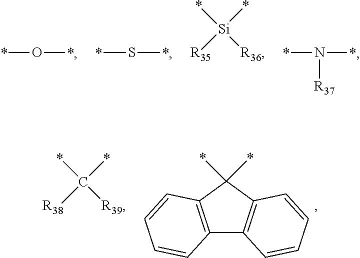

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, and e1 to e4 may each independently be 0 or 1. In Formula M-b, R₃₁ to R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring, and d1 to d4 may each independently be an integer from 0 to 4.

The compound represented by Formula M-b may be used as a blue phosphorescence dopant or a green phosphorescence dopant.

The compound represented by Formula M-b may be selected from any one among the compounds below. However, the compounds below are examples, and the compound represented by Formula M-b is not limited to the compounds represented below.

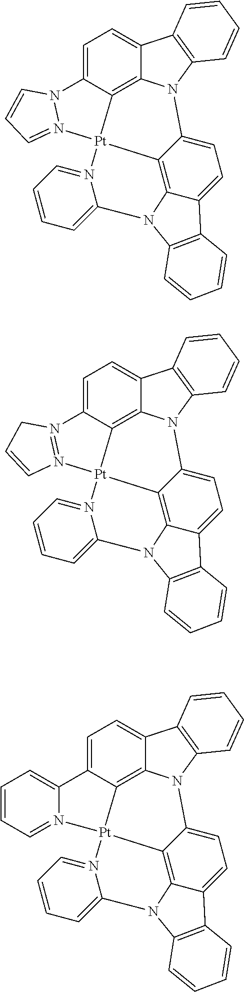

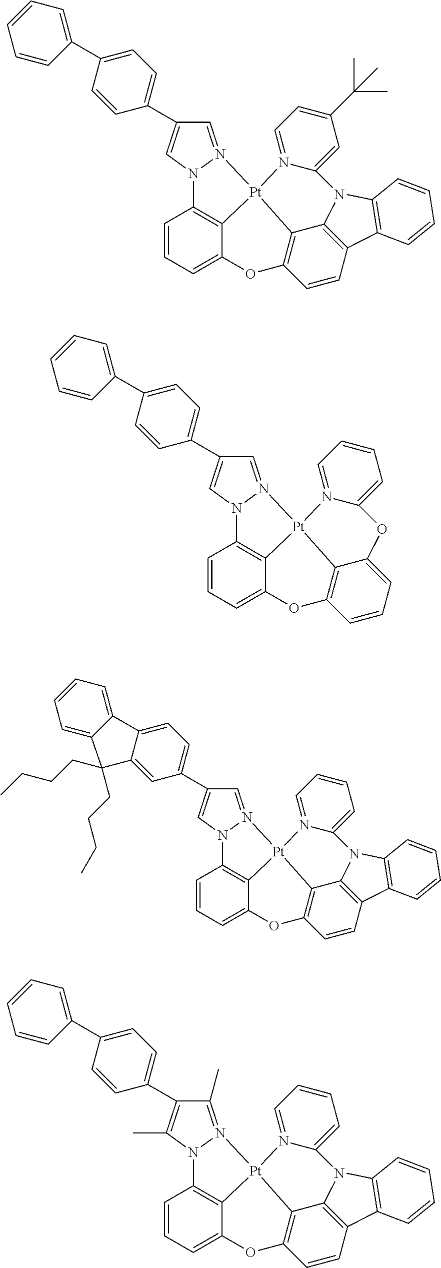

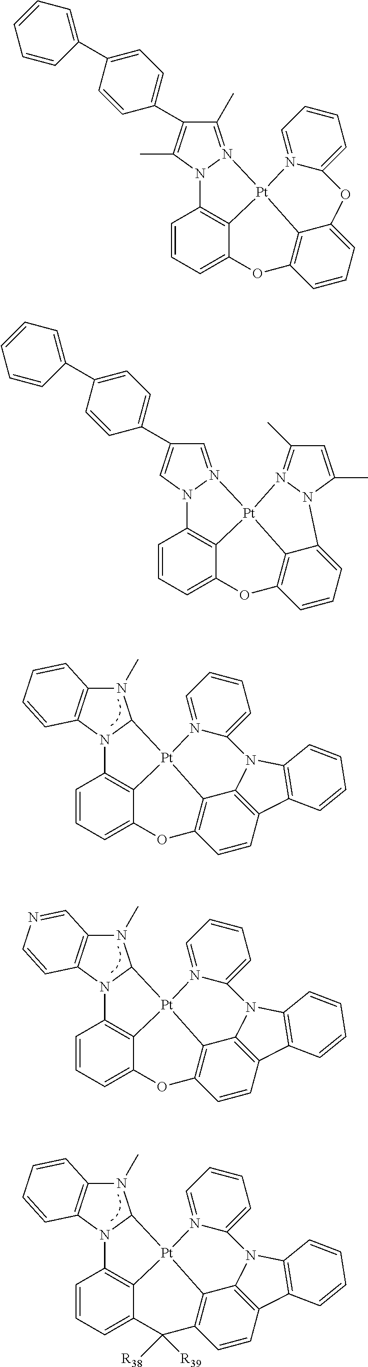

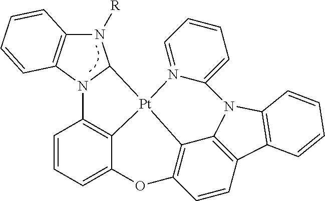

In the compounds above, R, R₃₈, and R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

At least one of the compounds below of the dopant material that include Pt as a central metal may be included.

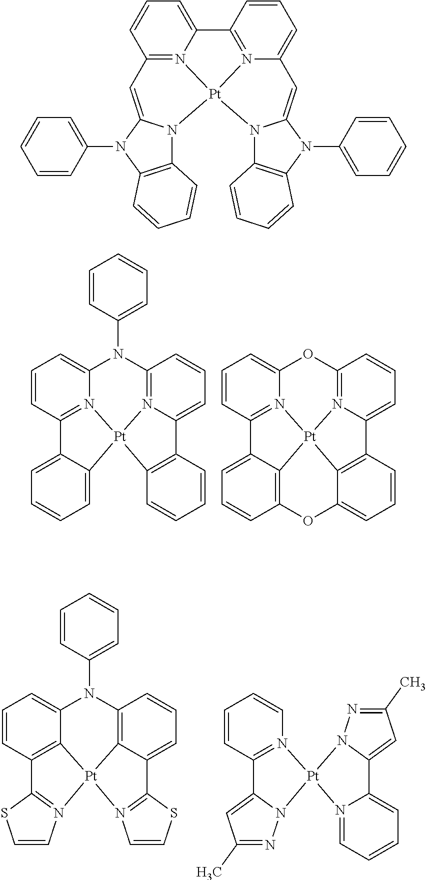

The emission layer EML may include a compound represented by any one among Formula F-a to Formula F-c below. The compounds represented by Formula F-a to Formula F-c below may be used as fluorescence dopant materials.

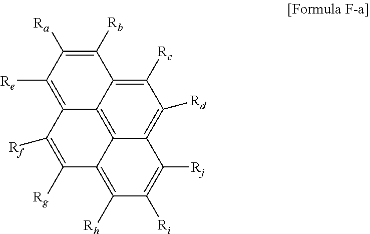

In Formula F-a, two selected from Rₐ to Rⱼ may each independently be substituted with *—NAr₁Ar₂. The remainder of Rₐ to Rⱼ not substituted with *—NAr₁Ar₂ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula F-a, in the moiety represented by *—NAr₁Ar₂, Ar₁ and Ar₂ may each independently be a substituted or unsubstituted aryl group of 6 to 30 ring-forming carbon atoms, or a substituted or unsubstituted heteroaryl group of 2 to 30 ring-forming carbon atoms. For example, at least one among Ar₁ and Ar₂ may be a heteroaryl group including 0 or S as a ring-forming atom.

The emission layer may include at least one among Compounds FD1 to FD22 below as a fluorescence dopant.

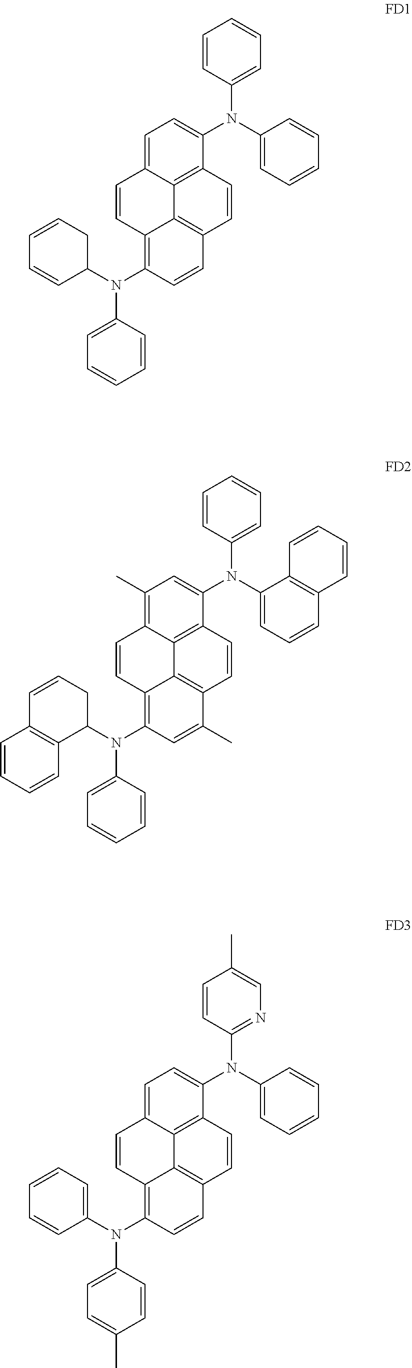 [SMILES: C1=CCC(N(c2ccccc2)c2ccc3ccc4c(N(c5ccccc5)c5ccccc5)ccc5ccc2c3c54)C=C1 ; Cc1cc(N(c2ccccc2)c2cccc3ccccc23)c2ccc3c(C)cc(N(c4ccccc4)C4CC=Cc5ccccc54)c4ccc1c2c34 ; Cc1ccc(N(c2ccccc2)c2ccc3ccc4c(N(c5ccccc5)c5ccc(C)cn5)ccc5ccc2c3c54)cc1]

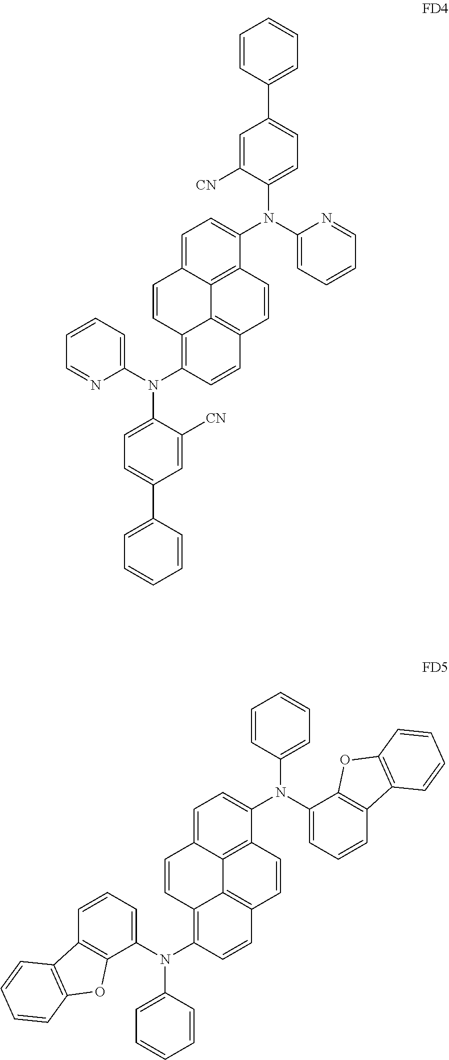

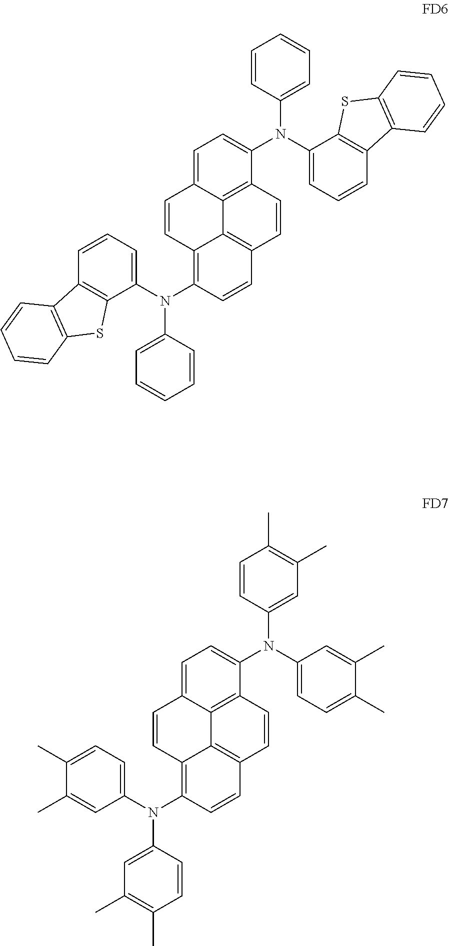

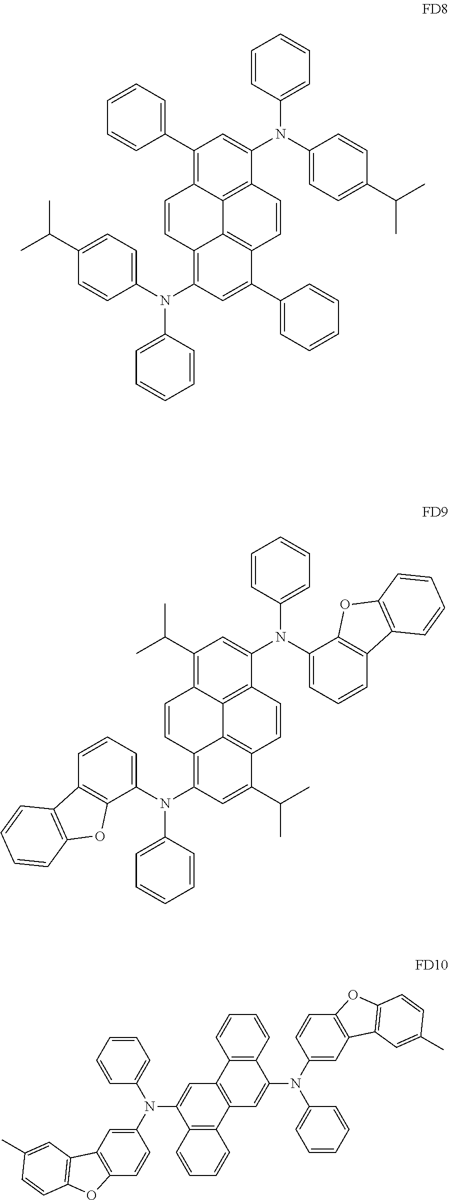

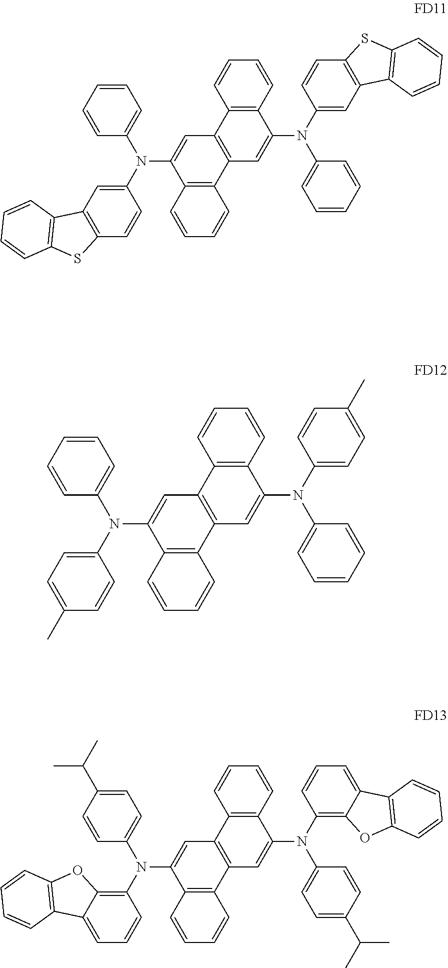 [SMILES: c1ccc(N(c2ccc3sc4ccccc4c3c2)c2cc3c4ccccc4c(N(c4ccccc4)c4ccc5sc6ccccc6c5c4)cc3c3ccccc23)cc1 ; Cc1ccc(N(c2ccccc2)c2cc3c4ccccc4c(N(c4ccccc4)c4ccc(C)cc4)cc3c3ccccc23)cc1 ; CC(C)c1ccc(N(c2cc3c4ccccc4c(N(c4ccc(C(C)C)cc4)c4cccc5c4oc4ccccc45)cc3c3ccccc23)c2cccc3c2oc2ccccc23)cc1]

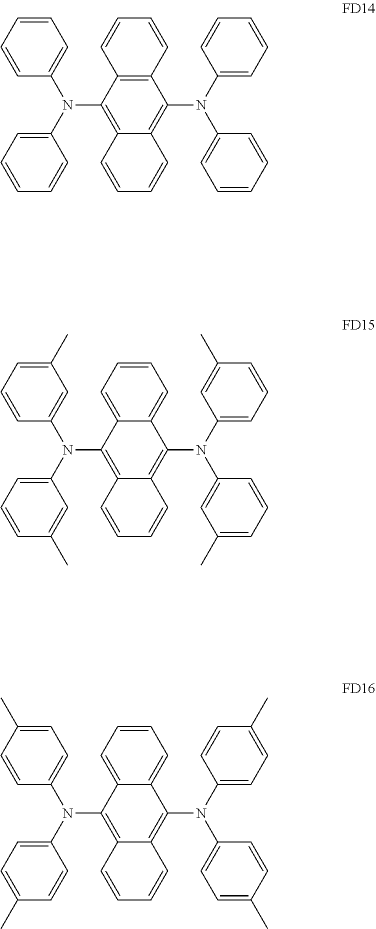 [SMILES: c1ccc(N(c2ccccc2)c2c3ccccc3c(N(c3ccccc3)c3ccccc3)c3ccccc23)cc1 ; Cc1cccc(N(c2cccc(C)c2)c2c3ccccc3c(N(c3cccc(C)c3)c3cccc(C)c3)c3ccccc23)c1 ; Cc1ccc(N(c2ccc(C)cc2)c2c3ccccc3c(N(c3ccc(C)cc3)c3ccc(C)cc3)c3ccccc23)cc1]

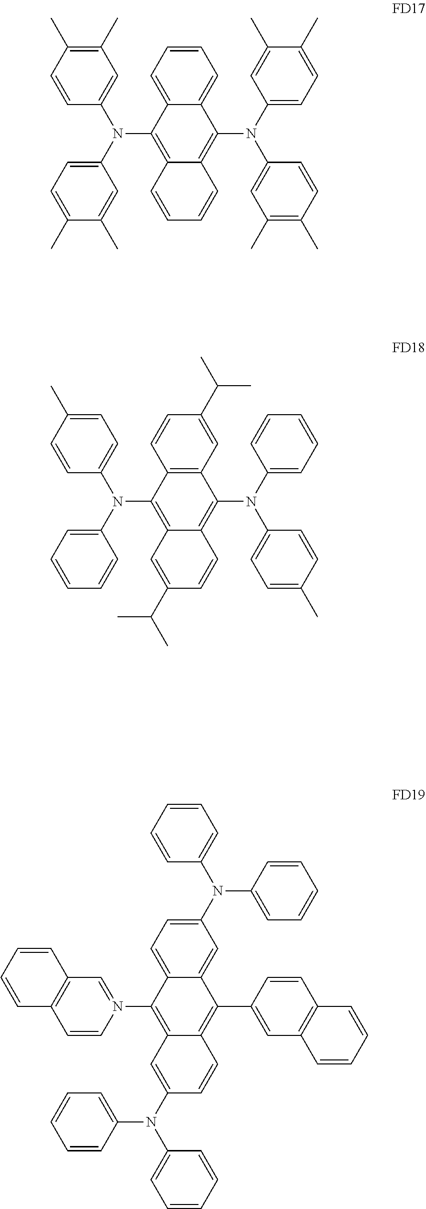

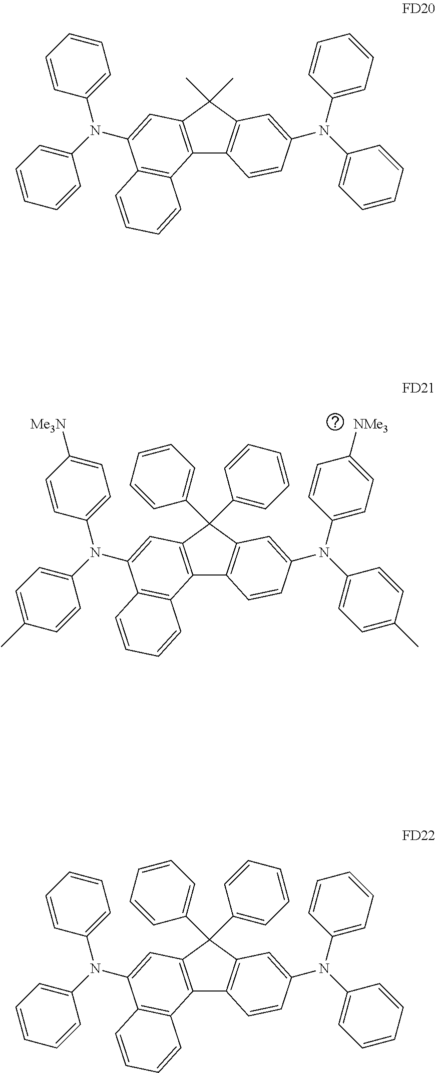

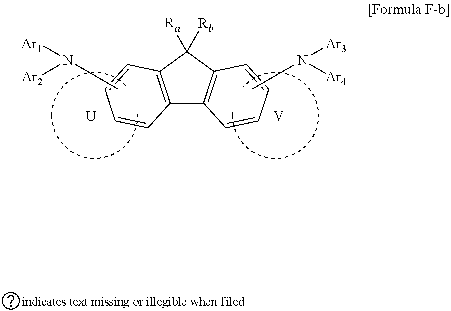

In Formula F-b, Rₐ and R_{b} may each independently be a hydrogen atom, a deuterium atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring.

In Formula F-b, U and V may each independently be a substituted or unsubstituted hydrocarbon ring of 5 to 30 ring-forming carbon atoms, or a substituted or unsubstituted heterocycle of 2 to 30 ring-forming carbon atoms.

In Formula F-b, the number of rings represented by U and V may each independently be 0 or 1. For example, in Formula F-b, if the number of U or V is 1, one ring may form a fused ring at the part designated by U or V, and if the number of U or V is 0, a ring may not be present at the part designated by U or V. For example, if the number of U is 0, and the number of V is 1, or if the number of U is 1, and the number of V is 0, a fused ring having the fluorene core of Formula F-b may be a ring compound with four rings. If the number of both U and V is 0, the fused ring having the fluorene core of Formula F-b may be a ring compound with three rings. If the number of U and V is each 1, a fused ring having the fluorene core of Formula F-b may be a ring compound with five rings.

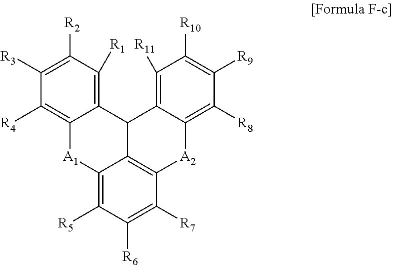

In Formula F-c, A₁ and A₂ may each independently be O, S, Se, or N(Rₘ), and Rₘ may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. In Formula F-c, R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring.

In Formula F-c, A₁ and A₂ may each independently be combined with the substituents of an adjacent group to form a fused ring. For example, if A₁ and A₂ are each N(Rₘ), A₁ may be combined with R₄ or R₅ to form a ring. For example, in Formula F-c, A₂ may be combined with R₇ or R₈ to form a ring.

In an embodiment, the emission layer EML may include as a dopant material, styryl derivatives (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and 4,4′-bis[2-(4-(N,N-diphenylamino)phenyl)vinyl]biphenyl (DPAVBi)), perylene and the derivatives thereof (for example, 2,5,8,11-tetra-t-butylperylene (TBP)), pyrene and the derivatives thereof (for example, 1,1-dipyrene, 1,4-dipyrenylbenzene, and 1,4-bis(N,N-diphenylamino)pyrene), etc.

The emission layer EML may include a phosphorescence dopant material. For example, the phosphorescence dopant may use a metal complex including iridium (Ir), platinum (Pt), osmium (Os), gold (Au), titanium (Ti), zirconium (Zr), hafnium (Hf), europium (Eu), terbium (Tb) or thulium (Tm). For example, iridium(III) bis(4,6-difluorophenylpyridinato-N,C2′)pincolinate) (FIrpic), bis(2,4-difluorophenylpyridinato)-tetrakis(1-pyrazolyl)borate iridium(III) (Fir6), or platinum octaethyl porphyrin (PtOEP) may be used as the phosphorescence dopant. However, embodiments are not limited thereto.

The emission layer EML may include a quantum dot material. The core of the quantum dot may be selected from a II-VI group compound, a III-VI group compound, a I-III-VI group compound, a III-V group compound, a IV-VI group compound, a IV group element, a IV group compound, and combinations thereof.

The II-VI group compound may be selected from the group consisting of: a binary compound selected from the group consisting of CdSe, CdTe, CdS, ZnS, ZnSe, ZnTe, ZnO, HgS, HgSe, HgTe, MgSe, MgS, and mixtures thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and mixtures thereof.

The III-VI group compound may include a binary compound such as In₂S₃, and In₂Se₃, a ternary compound such as InGaS₃, and InGaSe₃, or optional combinations thereof.

The I-III-VI group compound may be selected from a ternary compound selected from the group consisting of AgInS, AgInS₂, CuInS, CuInS₂, AgGaS₂, CuGaS₂, CuGaO₂, AgGaO₂, AgAlO₂ and mixtures thereof, or a quaternary compound such as AgInGaS₂, and CuInGaS₂.

The III-V group compound may be selected from the group consisting of a binary compound selected from the group consisting of GaN, GaP, GaAs, GaSb, AlN, AlP, AlAs, AlSb, InN, InP, InAs, InSb, and mixtures thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and mixtures thereof. The III-V group compound may further include a II group metal. For example, InZnP, etc. may be selected as a III-II-V group compound.

The IV-VI group compound may be selected from the group consisting of a binary compound selected from the group consisting of SnS, SnSe, SnTe, PbS, PbSe, PbTe, and mixtures thereof, a ternary compound selected from the group consisting of SnSeS, SnSeTe, SnSTe, PbSeS, PbSeTe, PbSTe, SnPbS, SnPbSe, SnPbTe, and mixtures thereof, and a quaternary compound selected from the group consisting of SnPbSSe, SnPbSeTe, SnPbSTe, and mixtures thereof. The IV group element may be selected from the group consisting of Si, Ge, and a mixture thereof. The IV group compound may be a binary compound selected from the group consisting of SiC, SiGe, and a mixture thereof.

The binary compound, the ternary compound, or the quaternary compound may be present at a uniform concentration in a particle or may be present at a partially different concentration distribution state in the same particle. A quantum dot may have a core/shell structure in which one quantum dot surrounds another quantum dot. An interface between the core and the shell may have a concentration gradient in which the concentration of an element present in the shell may decrease toward the core.

In an embodiment, the quantum dot may have the above-described core-shell structure including a core including a nanocrystal and a shell surrounding the core. The shell of the quantum dot may be a protection layer that prevents the chemical deformation of the core to maintain semiconductor properties and/or a charging layer that imparts the quantum dot with electrophoretic properties. The shell may have a single layer or a multilayer. The interface between the core and the shell may have a concentration gradient in which the concentration of an element present in the shell may decrease toward the core. Examples of the shell of the quantum dot may include a metal or non-metal oxide, a semiconductor compound, or combinations thereof.

For example, the metal or non-metal oxide may include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄ and NiO, or a ternary compound such as MgAl₂O₄, CoFe₂O₄, NiFe₂O₄ and CoMn₂O₄, but embodiments are not limited thereto.

The semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but embodiments are not limited thereto.

The quantum dot may have a full width of half maximum (FWHM) of an emission wavelength spectrum equal to or less than about 45 nm. For example, the quantum dot may have a FWHM of an emission wavelength spectrum equal to or less than about 40 nm. For example, the quantum dot may have a FWHM of an emission wavelength spectrum equal to or less than about 30 nm. Within these ranges, color purity or color reproducibility may be improved. Light emitted through the quantum dot may be emitted in all directions, and light viewing angle properties may be improved.

The shape of the quantum dot may be selected from among shapes which are used in the art, without specific limitation. For example, the quantum dot may have a spherical, a pyramidal, a multi-arm, or a cubic shape, or the quantum dot may be in the form of a nanoparticle, a nanotube, a nanowire, a nanofiber, or a nanoplate particle, etc.

The quantum dot may control the color of light emitted according to the particle size, and accordingly, the quantum dot may have various emission colors such as blue, red, and green.

In the light emitting diode ED of an embodiment, as shown in FIG. 2 to FIG. 6, the electron transport region ETR is provided on the emission layer EML. The electron transport region ETR may include at least one of a hole blocking layer HBL, an electron transport layer ETL, and an electron injection layer EIL. However, embodiments are not limited thereto.

The electron transport region ETR may have a single layer formed using a single material, a single layer formed using different materials, or a multilayer structure having layers formed using different materials.

For example, the electron transport region ETR may have a single layer structure of an electron injection layer EIL or an electron transport layer ETL, or a single layer structure formed using an electron injection material and an electron transport material. The electron transport region ETR may have a single layer structure formed using different materials, or a structure stacked from the emission layer EML of electron transport layer ETL/electron injection layer EIL, or hole blocking layer HBL/electron transport layer ETL/electron injection layer EIL, without limitation. A thickness of the electron transport region ETR may be, for example, in a range of about 1,000 Å to about 1,500 Å.

The electron transport region ETR may be formed using various methods such as a vacuum deposition method, a spin coating method, a cast method, a Langmuir-Blodgett (LB) method, an inkjet printing method, a laser printing method, and a laser induced thermal imaging (LITI) method.

The electron transport region ETR may include a compound represented by Formula ET-1 below.

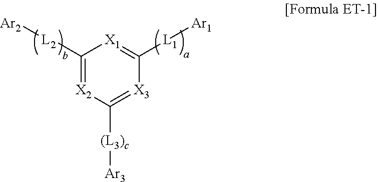

In Formula ET-1, at least one among X₁ to X₃ may be N, and the remainder of X₁ to X₃ may be C(Rₐ). Rₐ may be a hydrogen atom, a deuterium atom, a substituted or unsubstituted alkyl of 1 to 20 carbon atoms, a substituted or unsubstituted aryl group of 6 to 30 ring-forming carbon atoms, or a substituted or unsubstituted heteroaryl group of 2 to 30 ring-forming carbon atoms. In Formula ET-1, Ar₁ to Ar₃ may each independently be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-1, a to c may each independently be an integer from 0 to 10. In Formula ET-1, L₁ to L₃ may each independently be a direct linkage, a substituted or unsubstituted arylene group of 6 to 30 ring-forming carbon atoms, or a substituted or unsubstituted heteroarylene group of 2 to 30 ring-forming carbon atoms. In Formula ET-1, if a to c are 2 or more, L₁ to L₃ may each independently be a substituted or unsubstituted arylene group of 6 to 30 ring-forming carbon atoms, or a substituted or unsubstituted heteroarylene group of 2 to 30 ring-forming carbon atoms.

The electron transport region ETR may include an anthracene-based compound. However, embodiments are not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-ylphenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), and mixtures thereof, without limitation.

The electron transport region ETR may include at least one among Compounds ET1 to ET36 below.

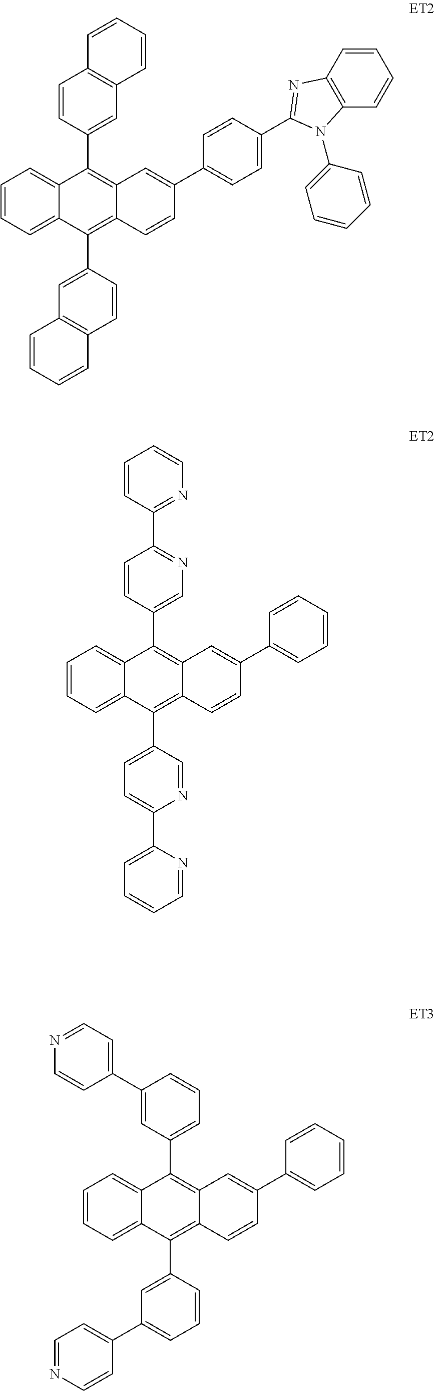 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

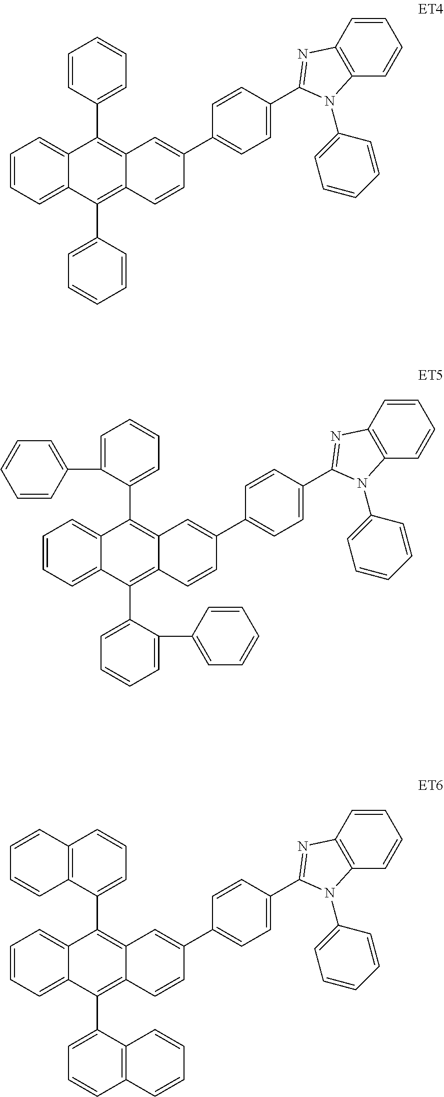 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

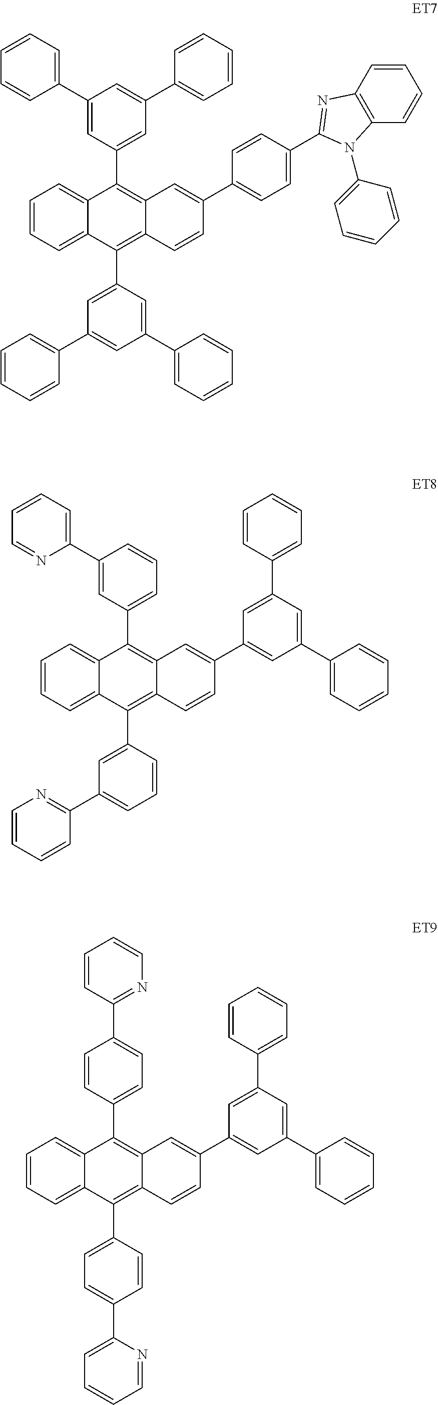 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

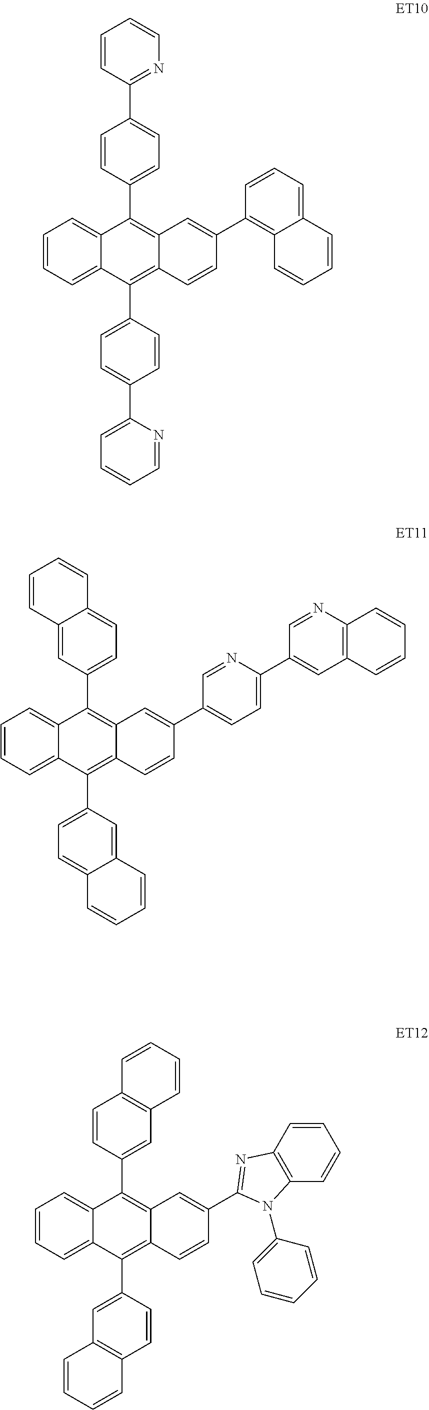 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

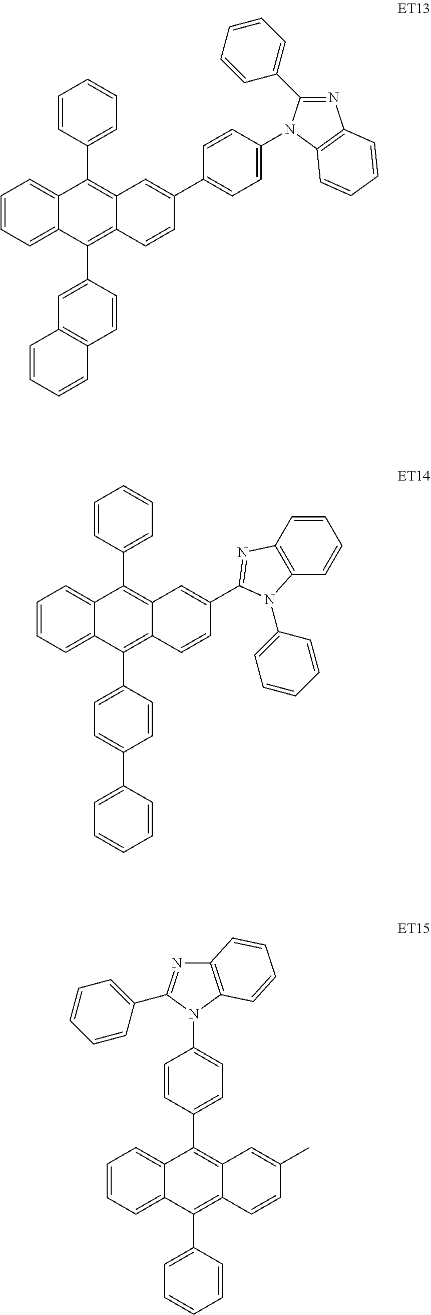 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

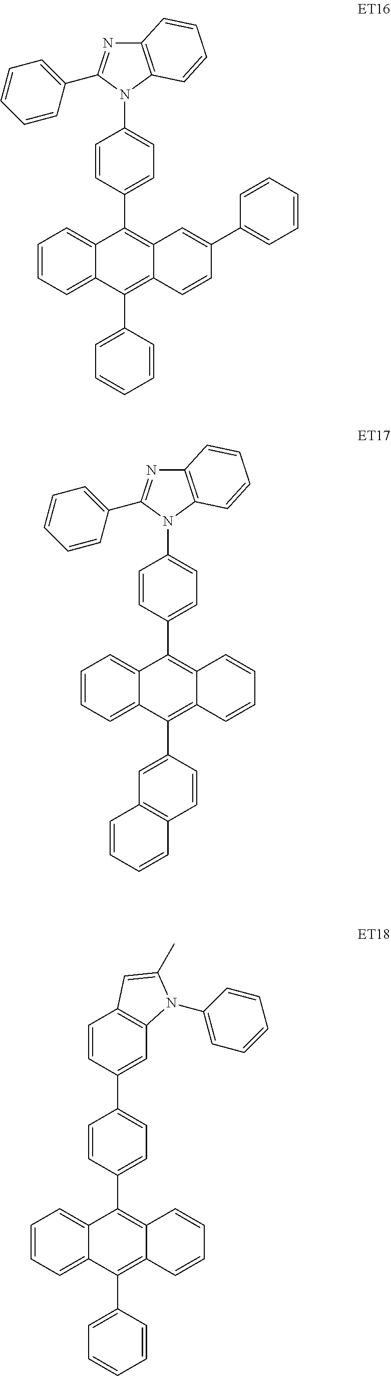 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1cc2ccc(-c3ccc(-c4c5ccccc5c(-c5ccccc5)c5ccccc45)cc3)cc2n1-c1ccccc1]

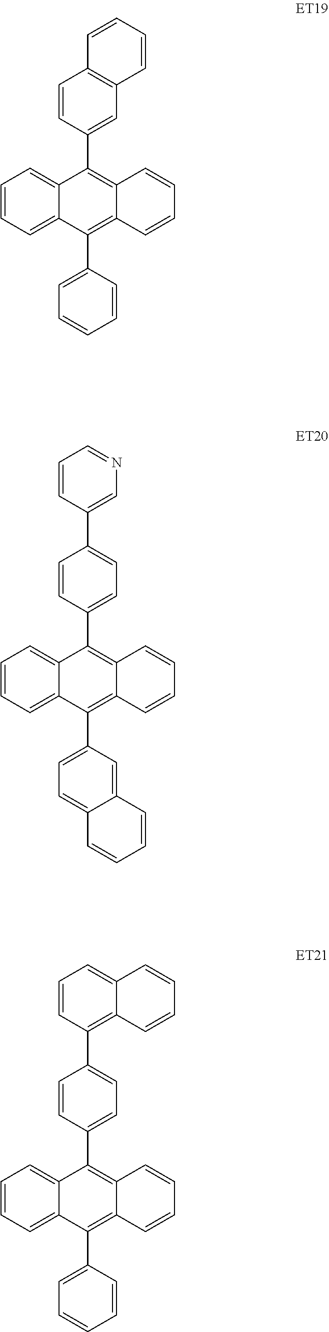

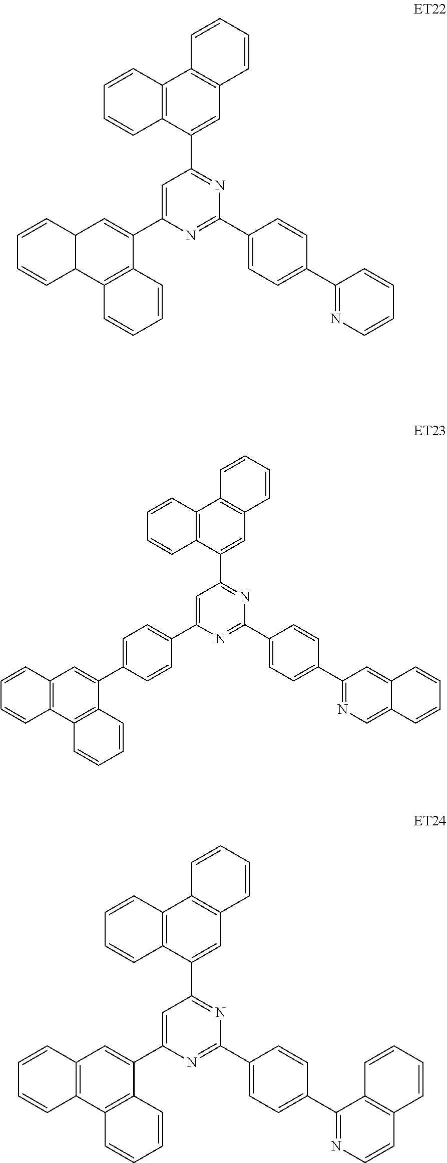 [SMILES: C1=CC2C=C(c3cc(-c4cc5ccccc5c5ccccc45)nc(-c4ccc(-c5ccccn5)cc4)n3)c3ccccc3C2C=C1 ; c1ccc2cc(-c3ccc(-c4nc(-c5ccc(-c6cc7ccccc7c7ccccc67)cc5)cc(-c5cc6ccccc6c6ccccc56)n4)cc3)ncc2c1 ; c1ccc2c(-c3ccc(-c4nc(-c5cc6ccccc6c6ccccc56)cc(-c5cc6ccccc6c6ccccc56)n4)cc3)nccc2c1]

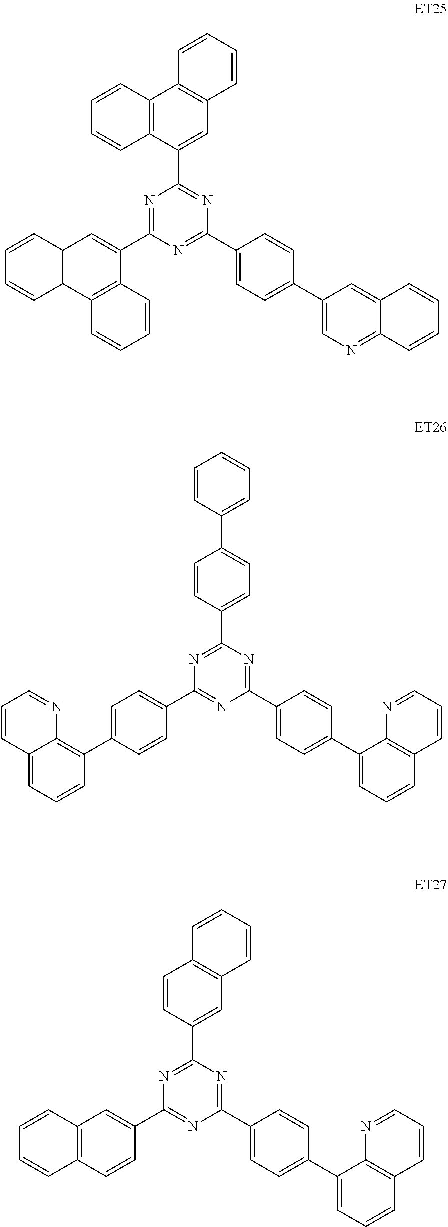 [SMILES: C1=CC2C=C(c3nc(-c4ccc(-c5cnc6ccccc6c5)cc4)nc(-c4cc5ccccc5c5ccccc45)n3)c3ccccc3C2C=C1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

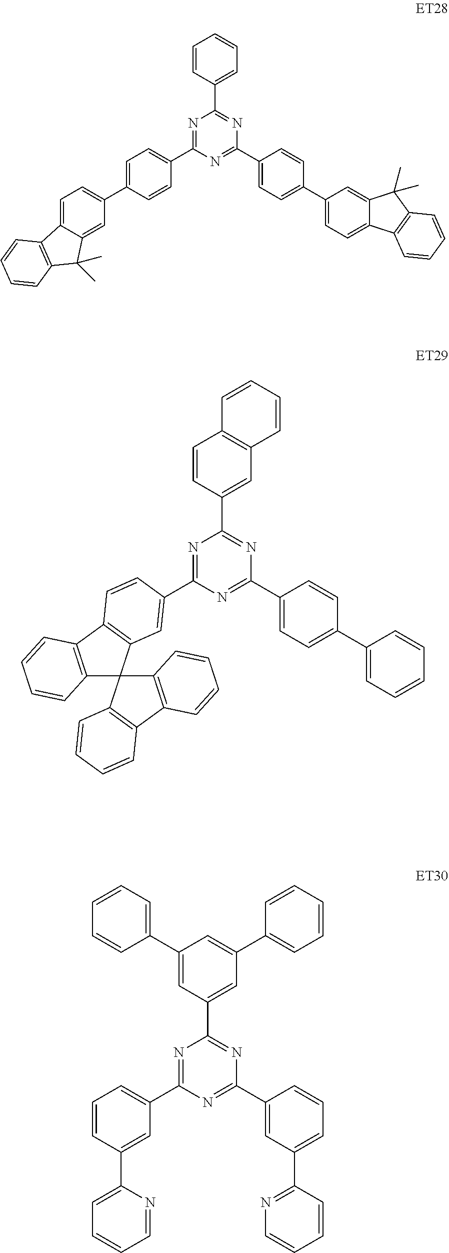 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3nc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

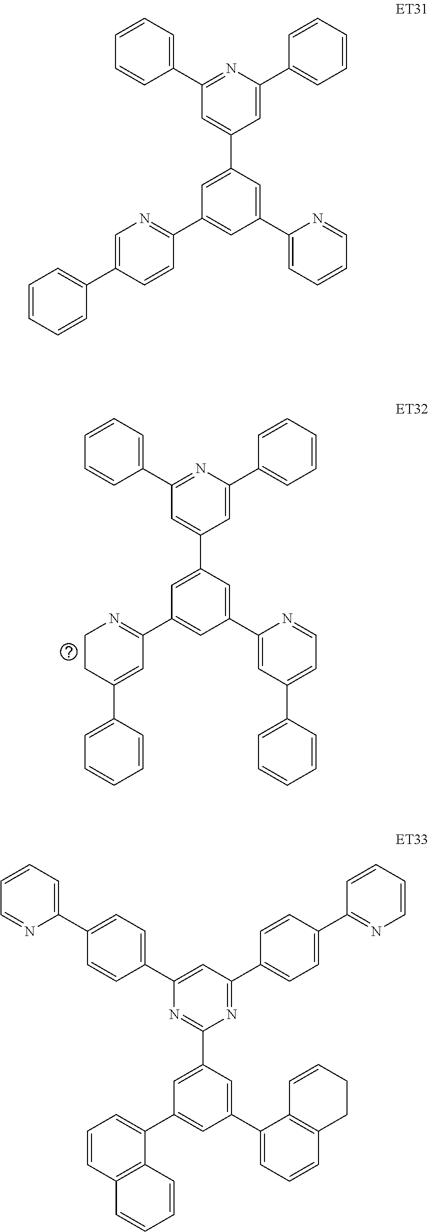 [SMILES: c1ccc(-c2ccc(-c3cc(-c4cc(-c5ccccc5)nc(-c5ccccc5)c4)cc(-c4ccccn4)c3)nc2)cc1 ; C1=C(c2ccccc2)CCN=C1c1cc(-c2cc(-c3ccccc3)nc(-c3ccccc3)c2)cc(-c2cc(-c3ccccc3)ccn2)c1 ; C1=Cc2c(cccc2-c2cc(-c3nc(-c4ccc(-c5ccccn5)cc4)cc(-c4ccc(-c5ccccn5)cc4)n3)cc(-c3cccc4ccccc34)c2)CC1]

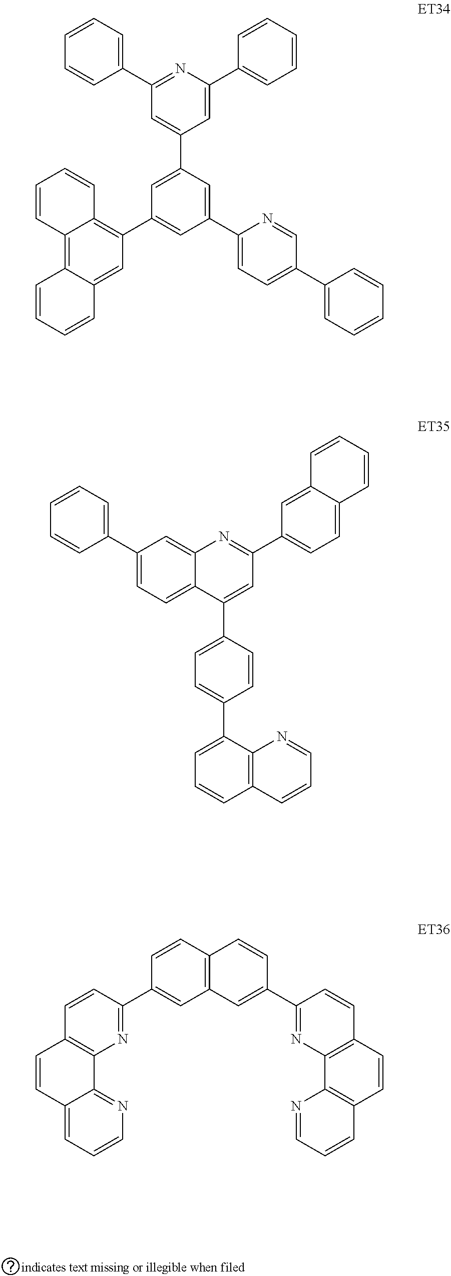 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

The electron transport region ETR may include a metal halide such as LiF, NaCl, CsF, RbCl, RbI, CuI and KI, a lanthanide such as Yb, or a co-depositing material of the metal halide and the lanthanide. For example, the electron transport region ETR may include KI:Yb, RbI:Yb, etc., as a co-depositing material. The electron transport region ETR may use a metal oxide such as Li₂O and BaO, or 8-hydroxy-lithium quinolate (Liq). However, embodiments are not limited thereto. The electron transport region ETR also may be formed using a mixture of an electron transport material and an insulating organo metal salt. The organo metal salt may be a material having an energy band gap of about 4 eV or more. For example, the organo metal salt may include metal acetates, metal benzoates, metal acetoacetates, metal acetylacetonates, or metal stearates.

The electron transport region ETR may include at least one of 2,9-dimethyl-4,7-diphenyl-1,10-phenanthroline (BCP), and 4,7-diphenyl-1,10-phenanthroline (Bphen) in addition to the aforementioned materials. However, embodiments are not limited thereto.

The electron transport region ETR may include the compounds of the electron transport region in at least one among an electron injection layer EIL, an electron transport layer ETL, and a hole blocking layer HBL.

If the electron transport region ETR includes an electron transport layer ETL, a thickness of the electron transport layer ETL may be in a range of about 100 Å to about 1,000 Å. For example, the thickness of the electron transport layer ETL may be in a range of about 150 Å to about 500 Å. If the thickness of the electron transport layer ETL satisfies the above-described range, satisfactory electron transport properties may be obtained without substantial increase of a driving voltage. If the electron transport region ETR includes an electron injection layer EIL, a thickness of the electron injection layer EIL may be in a range of about 1 Å to about 100 Å. For example, the thickness of the electron injection layer EIL may be in a range of about 3 Å to about 90 Å. If the thickness of the electron injection layer EIL satisfies the above described range, satisfactory electron injection properties may be obtained without inducing substantial increase of a driving voltage.

The second electrode EL2 is provided on the electron transport region ETR. The second electrode EL2 may be a common electrode. The second electrode EL2 may be a cathode or an anode, but embodiments are not limited thereto. For example, if the first electrode EL1 is an anode, the second cathode EL2 may be a cathode, and if the first electrode EL1 is a cathode, the second electrode EL2 may be an anode.

The second electrode EL2 may be a transmissive electrode, a transflective electrode, or a reflective electrode. If the second electrode EL2 is a transmissive electrode, the second electrode EL2 may include a transparent metal oxide, for example, ITO, IZO, ZnO, ITZO, etc.

If the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, compounds thereof, or mixtures thereof (for example, AgMg, AgYb, or MgYb). In an embodiment, the second electrode EL2 may have a multilayered structure including a reflective layer or a transflective layer formed using the above-described materials and a transparent conductive layer formed using ITO, IZO, ZnO, ITZO, etc. For example, the second electrode EL2 may include the aforementioned metal materials, combinations of two or more metal materials selected from the aforementioned metal materials, or oxides of the aforementioned metal materials.

Though not shown, the second electrode EL2 may be electrically connected to an auxiliary electrode. If the second electrode EL2 is electrically connected to the auxiliary electrode, the resistance of the second electrode EL2 may decrease.

In an embodiment, the light emitting diode ED may further include a capping layer CPL disposed on the second electrode EL2. The capping layer CPL may include a multilayer or a single layer.

In an embodiment, the capping layer CPL may include an organic layer or an inorganic layer. For example, if the capping layer CPL includes an inorganic material, the inorganic material may include an alkali metal compound such as LiF, an alkaline earth metal compound such as MgF₂, SiON, SiNx, SiOy, etc.

For example, if the capping layer CPL includes an organic material, the organic material may include α-NPD, NPB, TPD, m-MTDATA, Alq₃, CuPc, N4,N4,N4′,N4′-tetra(biphenyl-4-yl) biphenyl-4,4′-diamine (TPD15), 4,4′,4″-tris(carbazol sol-9-yl) triphenylamine (TCTA), etc., or includes an epoxy resin, or acrylate such as methacrylate. The capping layer CPL may include at least one among Compounds P1 to P5 below, but embodiments are not limited thereto.

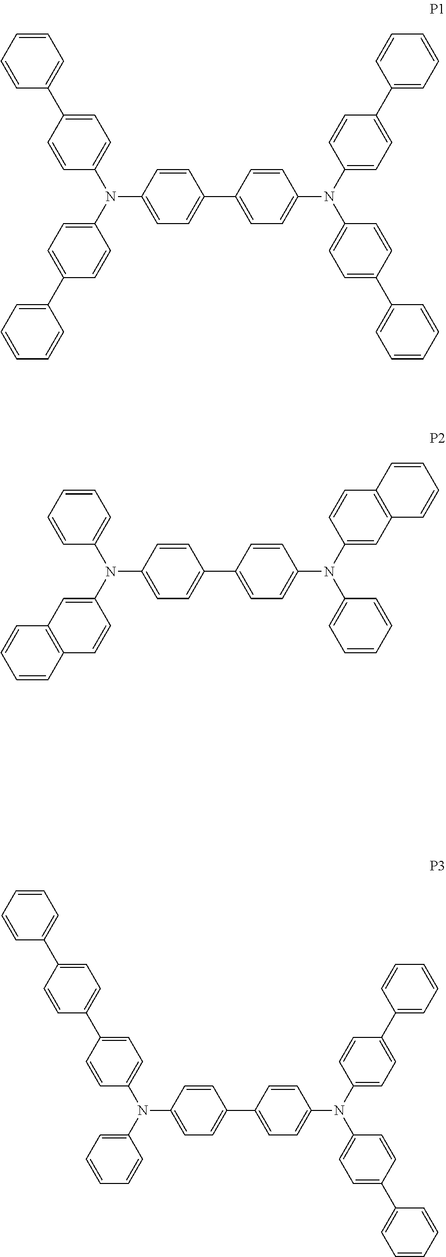 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

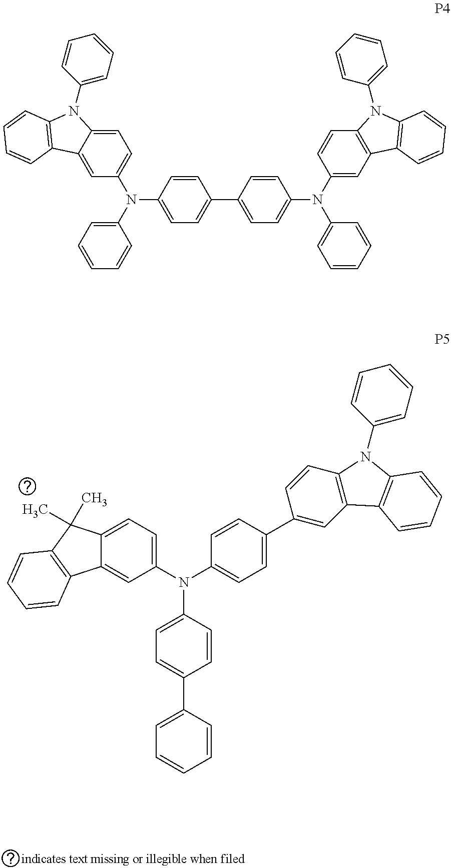 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

A refractive index of the capping layer CPL may be equal to or greater than about 1.6. For example, the refractive index of the capping layer CPL with respect to light in a wavelength range of about 550 nm to about 660 nm may be equal to or greater than about 1.6.

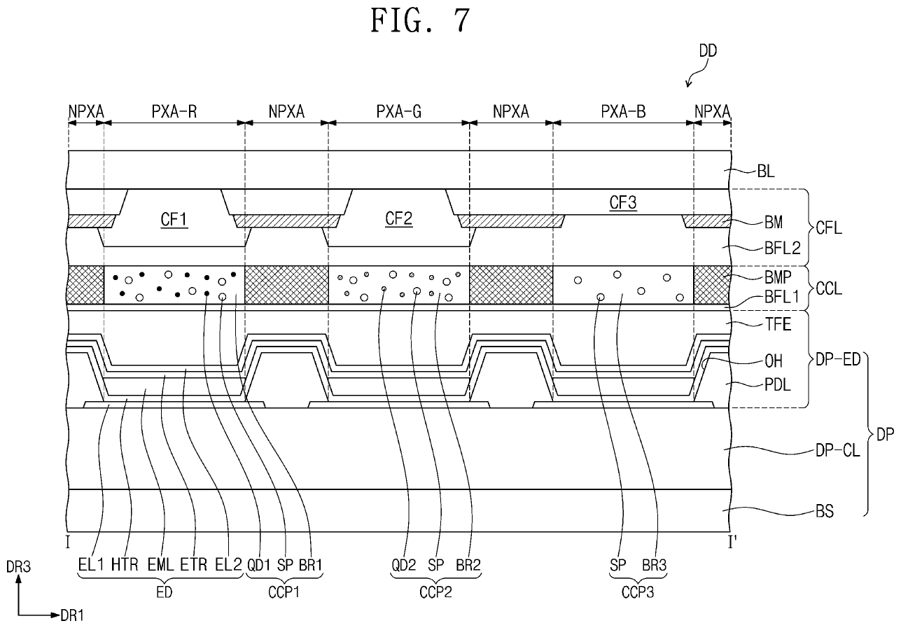

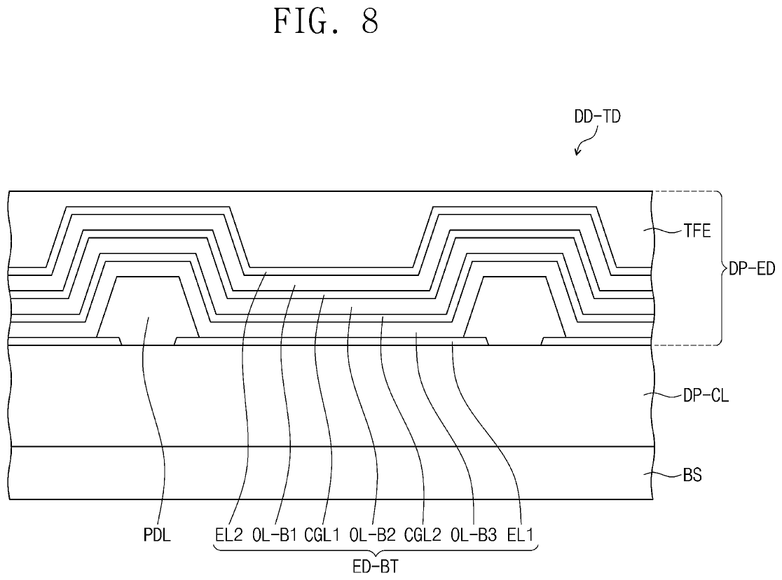

FIG. 7 and FIG. 8 are schematic cross-sectional views of display devices according to embodiments. Hereinafter, in the explanation on the display devices of embodiments, referring to FIG. 7 and FIG. 8, the overlapping parts with the explanation on FIG. 1 to FIG. 6 will not be explained again, and the different features will be explained.

Referring to FIG. 7, the display device DD according to an embodiment may include a display panel DP including a display element layer DP-ED, a light controlling layer CCL disposed on the display panel DP, and a color filter layer CFL.

In an embodiment shown in FIG. 7, the display panel DP includes a base layer BS, a circuit layer DP-CL provided on the base layer BS, and a display element layer DP-ED, and the display element layer DP-ED may include a light emitting diode ED.

The light emitting diode ED may include a first electrode EL1, a hole transport region HTR disposed on the first electrode EL1, an emission layer EML disposed on the hole transport region HTR, an electron transport region ETR disposed on the emission layer EML, and a second electrode EL2 disposed on the electron transport region ETR. The same structures of the light emitting diodes of FIG. 3 to FIG. 6 may be applied to the structure of the light emitting diode ED shown in FIG. 7.

Referring to FIG. 7, the emission layer EML may be disposed in an opening part OH defined in a pixel definition layer PDL. For example, the emission layer EML divided by the pixel definition layer PDL and correspondingly provided to each of emitting areas PXA-R, PXA-G, and PXA-B may emit light in a same wavelength region. In the display device DD of an embodiment, the emission layer EML may emit blue light. Although not shown in the drawings, in an embodiment, the emission layer EML may be provided as a common layer for all emitting areas PXA-R, PXA-G, and PXA-B.

The light controlling layer CCL may be disposed on the display panel DP. The light controlling layer CCL may include a light converter. The light converter may include a quantum dot or a phosphor. The light converter may transform the wavelength of light provided and emit the transformed light. For example, the light controlling layer CCL may be a layer including a quantum dot or a layer including a phosphor.

The light controlling layer CCL may include multiple light controlling parts CCP1, CCP2, and CCP3. The light controlling parts CCP1, CCP2, and CCP3 may be separated from one another.

Referring to FIG. 7, a partition pattern BMP may be disposed between the separated light controlling parts CCP1, CCP2, and CCP3, but embodiments are not limited thereto. In FIG. 7, the partition pattern BMP is shown to not overlap with the light controlling parts CCP1, CCP2, and CCP3, but at least a portion of the edge of the light controlling parts CCP1, CCP2, and CCP3 may overlap with the partition pattern BMP.

The light controlling layer CCL may include a first light controlling part CCP1 including a first quantum dot QD1 converting first color light provided from the light emitting diode ED into second color light, a second light controlling part CCP2 including a second quantum dot QD2 converting first color light into third color light, and a third light controlling part CCP3 transmitting first color light.

In an embodiment, the first light controlling part CCP1 may provide red light which is the second color light, and the second light controlling part CCP2 may provide green light which is the third color light. The third light controlling part CCP3 may transmit and provide blue light which is the first color light provided from the light emitting diode ED. For example, the first quantum dot QD1 may be a red quantum dot, and the second quantum dot QD2 may be a green quantum dot. The same description as given to the quantum dots described above may be applied to quantum dots QD1 and QD2.

The light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include a quantum dot but include the scatterer SP.

The scatterer SP may be an inorganic particle. For example, the scatterer SP may include at least one among TiO₂, ZnO, Al₂O₃, SiO₂, and hollow silica. The scatterer SP may include at least one among TiO₂, ZnO, Al₂O₃, SiO₂, and hollow silica, or may be a mixture of two or more materials selected among TiO₂, ZnO, Al₂O₃, SiO₂, and hollow silica.

Each of the first light controlling part CCP1, the second light controlling part CCP2, and the third light controlling part CCP3 may include base resins BR1, BR2, and BR3 dispersing the quantum dots QD1 and QD2 and the scatterer SP. In an embodiment, the first light controlling part CCP1 may include the first quantum dot QD1 and the scatterer SP dispersed in the first base resin BR1, the second light controlling part CCP2 may include the second quantum dot QD2 and the scatterer SP dispersed in the second base resin BR2, and the third light controlling part CCP3 may include the scatterer particle SP dispersed in the third base resin BR3. The base resins BR1, BR2, and BR3 are mediums in which the quantum dots QD1 and QD2 and the scatterer SP are dispersed, and may be composed of various resin compositions which may be generally referred to as a binder. For example, the base resins BR1, BR2, and BR3 may be acrylic resins, urethane-based resins, silicone-based resins, epoxy-based resins, etc. The base resins BR1, BR2, and BR3 may be transparent resins. In an embodiment, the first base resin BR1, the second base resin BR2, and the third base resin BR3 may be the same as or different from each other.

The light controlling layer CCL may include a barrier layer BFL1. The barrier layer BFL1 may prevent penetration of moisture and/or oxygen (hereinafter, will be referred to as “humidity/oxygen”). The barrier layer BFL1 may be disposed on the light controlling parts CCP1, CCP2, and CCP3 to block the exposure of the light controlling parts CCP1, CCP2, and CCP3 to humidity/oxygen. The barrier layer BFL1 may cover the light controlling parts CCP1, CCP2, and CCP3. A barrier layer BFL2 may be provided between the light controlling parts CCP1, CCP2, and CCP3 and a color filter layer CFL.

The barrier layers BFL1 and BFL2 may include at least one inorganic layer. For example, the barrier layers BFL1 and BFL2 may be formed by including an inorganic material. For example, the barrier layers BFL1 and BFL2 may be formed by including silicon nitride, aluminum nitride, zirconium nitride, titanium nitride, hafnium nitride, tantalum nitride, silicon oxide, aluminum oxide, titanium oxide, tin oxide, cerium oxide and silicon oxynitride or a metal thin film securing light transmittance. The barrier layers BFL1 and BFL2 may further include an organic layer. The barrier layers BFL1 and BFL2 may be composed of a single layer or of multiple layers.

In the display device DD of an embodiment, the color filter layer CFL may be disposed on the light controlling layer CCL. For example, the color filter layer CFL may be disposed directly on the light controlling layer CCL. In an embodiment, the barrier layer BFL2 may be omitted.

The color filter layer CFL may include a light blocking part BM and filters CF1, CF2, and CF3. The color filter layer CFL may include a first filter CF1 transmitting second color light, a second filter CF2 transmitting third color light, and a third filter CF3 transmitting first color light. For example, the first filter CF1 may be a red filter, the second filter CF2 may be a green filter, and the third filter CF3 may be a blue filter. Each of the filters CF1, CF2, and CF3 may include a polymer photosensitive resin and a pigment or dye. The first filter CF1 may include a red pigment or dye, the second filter CF2 may include a green pigment or dye, and the third filter CF3 may include a blue pigment or dye. However, embodiments are not limited thereto, and the third filter CF3 may not include the pigment or dye. The third filter CF3 may include a polymer photosensitive resin and not include a pigment or dye. The third filter CF3 may be transparent. The third filter CF3 may be formed using a transparent photosensitive resin.

In an embodiment, the first filter CF1 and the second filter CF2 may each be a yellow filter. The first filter CF1 and the second filter CF2 may be provided in one body without distinction.

The light blocking part BM may be a black matrix. The light blocking part BM may be formed by including an organic light blocking material or an inorganic light blocking material including a black pigment or black dye. The light blocking part BM may prevent light leakage and may divide the boundaries among adjacent filters CF1, CF2, and CF3. In an embodiment, the light blocking part BM may be formed as a blue filter.

Each of the first to third filters CF1, CF2, and CF3 may be disposed to respectively correspond to each of a red emitting area PXA-R, a green emitting area PXA-G, and a blue emitting area PXA-B.

A base substrate BL may be disposed on the color filter layer CFL. The base substrate BL may be a member providing a base surface on which the color filter layer CFL, the light controlling layer CCL, etc. are disposed. The base substrate BL may be a glass substrate, a metal substrate, a plastic substrate, etc. However, embodiments are not limited thereto, and the base substrate BL may include an inorganic layer, an organic layer, or a composite material layer. Although not shown in the drawings, the base substrate BL may be omitted in an embodiment.

FIG. 8 is a schematic cross-sectional view showing a portion of the display device according to an embodiment. In FIG. 8, the schematic cross-sectional view of a portion corresponding to the display panel DP in FIG. 7 is shown. In a display device DD-TD of an embodiment, the light emitting diode ED-BT may include light emitting structures OL-B1, OL-B2, and OL-B3. The light emitting diode ED-BT may include a first electrode EL1 and an oppositely disposed second electrode EL2, and the light emitting structures OL-B1, OL-B2, and OL-B3 may be stacked in order in a thickness direction and provided between the first electrode EL1 and the second electrode EL2. Each of the light emitting structures OL-B1, OL-B2, and OL-B3 may include an emission layer EML (FIG. 7), and a hole transport region HTR, and an electron transport region ETR, with the emission layer EML (FIG. 7) disposed therebetween.

For example, the light emitting diode ED-BT included in the display device DD-TD of an embodiment may be a light emitting diode having a tandem structure including multiple emission layers.

In an embodiment shown in FIG. 8, light emitted from the light emitting structures OL-B1, OL-B2, and OL-B3 may be all blue light. However, embodiments are not limited thereto, and the wavelength regions of light emitted from the light emitting structures OL-B1, OL-B2, and OL-B3 may be different from each other. For example, the light emitting diode ED-BT including the multiple light emitting structures OL-B1, OL-B2, and OL-B3 emitting light in different wavelength regions may emit white light.

Charge generating layers CGL1 and CGL2 may be disposed between neighboring light emitting structures OL-B1, OL-B2, and OL-B3. The charge generating layers CGL1 and CGL2 may each include a p-type charge generating layer and/or an n-type charge generating layer.

At least one of light emitting structures OL-B1, OL-B2, and OL-B3 included in the display device DD-TD of an embodiment may include the above-described amine compound of an embodiment.

The light emitting diode ED according to an embodiment may include the amine compound of an embodiment in at least one functional layer disposed between the first electrode EL1 and the second electrode EL2 and may show improved emission efficiency and improved life characteristics. The light emitting diode ED according to an embodiment may include the amine compound of an embodiment in at least one of a hole transport region HTR, an emission layer EML, and an electron transport region ETR disposed between the first electrode EL1 and the second electrode EL2, or in a capping layer CPL.

For example, the amine compound according to an embodiment may be included in the hole transport region HTR of the light emitting diode ED of an embodiment, and the light emitting diode of an embodiment may show excellent emission efficiency and long-life characteristics.

The amine compound of an embodiment includes a biphenyl group as a linking moiety and amine groups bonded to each of the benzene rings of the biphenyl group, and at least one of the amine groups includes a dibenzoheterole group, and accordingly, excellent hole transport capacity and improved material stability may be achieved. When the amine compound of an embodiment is used as a material for a light emitting diode, emission efficiency and life of the light emitting diode may be improved.

Hereinafter, the amine compound according to an embodiment and the light emitting diode of an embodiment will be explained referring to embodiments and comparative embodiments. The embodiments below are only examples to assist the understanding of the disclosure, and the scope of the disclosure is not limited thereto.

### EXAMPLES

1. Synthesis of Amine Compound

First, the synthesis method of an amine compound according to an embodiment will be explained in particular illustrating the synthesis methods of Compound A11, Compound A16, Compound B19, Compound D27, Compound I7, Compound L30, Compound Q14, Compound Q30, Compound T13, and Compound X2 in Compound Groups. The synthesis methods of the amine compounds explained hereinafter are embodiments, and the synthesis method of the amine compound according to an embodiment is not limited to the embodiments below.

<Synthesis of Compound A11>

Amine Compound A1 according to an embodiment may be synthesized, for example, by the steps of Reaction 1 below.

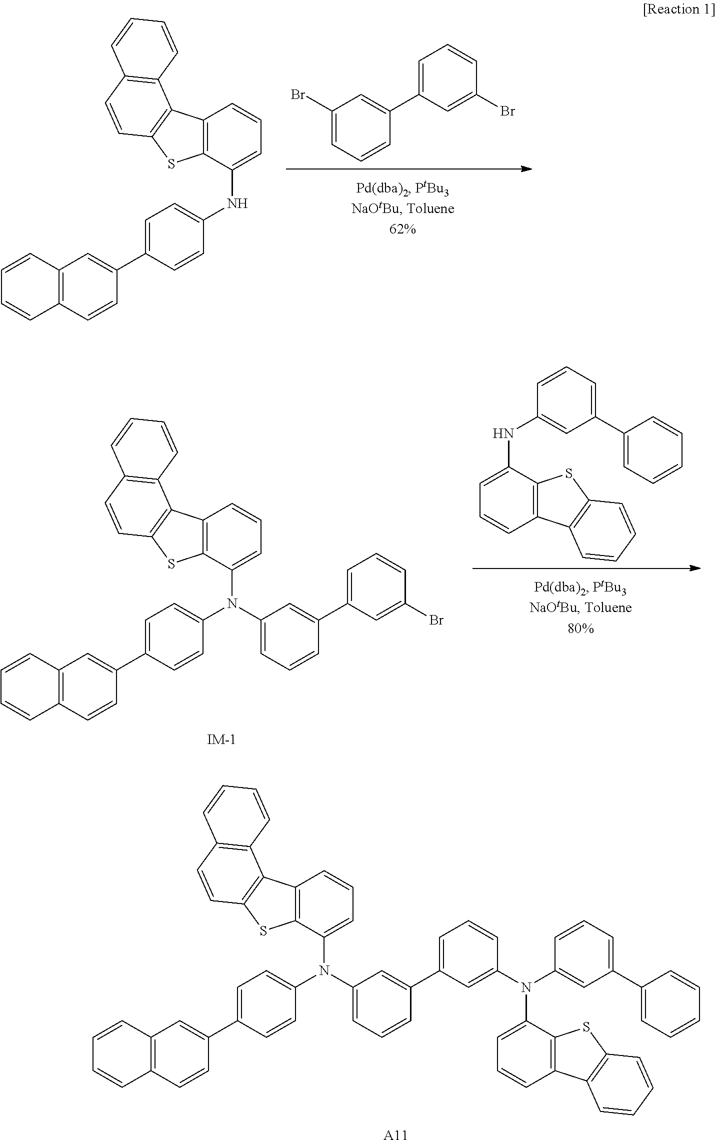

(Synthesis of Intermediate IM-1)

Under an argon (Ar) atmosphere, to a 500 ml, three-neck flask, 10.0 g (22.1 mmol) of N-(4-(naphthalen-2-yl)phenyl)benzo[b]naphtho[1,2-d]thiophen-8-amine, 0.64 g (0.05 equiv, 1.1 mmol) of Pd(dba)₂, 2.13 g (1.0 equiv, 22.1 mmol) of NaO^{t}Bu, 221 mL of toluene, 17.27 g (2.5 equiv, 55.4 mmol) of 3,3′-dibromo-1,1′-biphenyl and 0.90 g (0.2 equiv, 4.4 mmol) of P^{t}Bu₃ were added in order, and stirred while heating and refluxing. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered, an organic layer was concentrated, and the crude product thus obtained was separated by silica gel column chromatography (using a mixture solvent of hexane and toluene as a developing layer) to obtain Intermediate IM-1 (9.37 g, yield 62%).

FAB-MS was measured, mass number m/z=681 was observed as a molecular ion peak, and Intermediate IM-1 was identified.

(Synthesis of Compound A11)

Under an argon atmosphere, to a 300 ml, three-neck flask, 9.37 g (13.7 mmol) of Intermediate IM-1, 0.39 g (0.05 equiv, 0.7 mmol) of Pd(dba)₂, 1.32 g (1.0 equiv, 13.7 mmol) of NaO^{t}Bu, 137 mL of toluene, 4.82 g (1.0 equiv, 13.7 mmol) of N-([1,1′-biphenyl]-3-yl)dibenzo[b,d]thiophen-4-amine and 0.56 g (0.2 equiv, 2.8 mmol) of P^{t}Bu₃ were added in order, and stirred while heating and refluxing. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered, an organic layer was concentrated, and the crude product thus obtained was separated by silica gel column chromatography (using a mixture solvent of hexane and toluene as a developing layer) to obtain Compound A11 (10.47 g, yield 80%).

FAB-MS was measured, mass number m/z=952 was observed as a molecular ion peak, and Compound A11 was identified.

<Synthesis of Compound A16>

Amine Compound A16 according to an embodiment may be synthesized, for example, by the steps of Reaction 2 below.

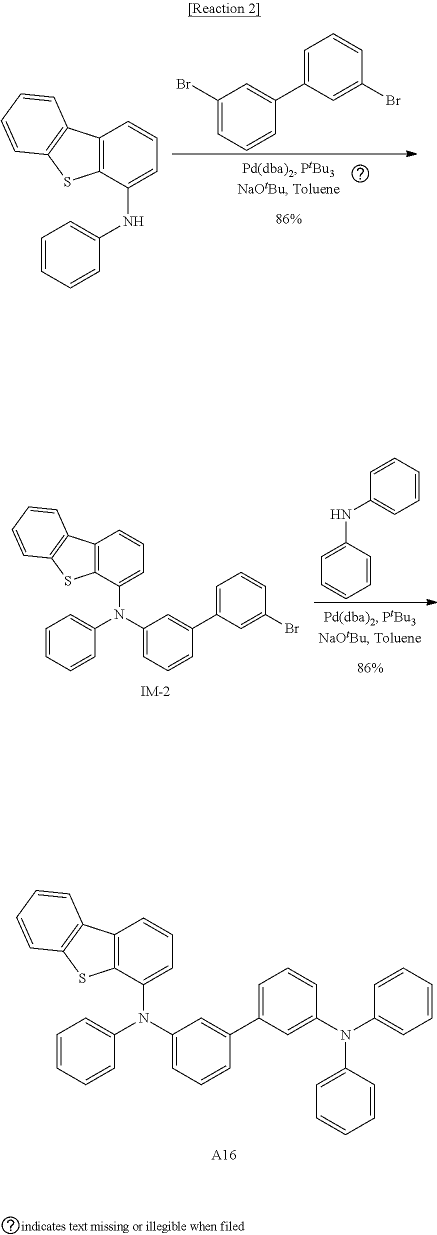

(Synthesis of Intermediate IM-2)

Under an argon atmosphere, to a 1,000 ml, three-neck flask, 10.0 g (36.3 mmol) of N-phenyldibenzo[b,d]thiophen-4-amine, 1.04 g (0.05 equiv, 1.82 mmol) of Pd(dba)₂, 3.49 g (1.0 equiv, 36.3 mmol) of NaO^{t}Bu, 363 mL of toluene, 28.33 g (2.5 equiv, 90.8 mmol) of 3,3′-dibromo-1,1′-biphenyl and 1.47 g (0.2 equiv, 7.3 mmol) of P^{t}Bu₃ were added in order, and stirred while heating and refluxing. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered, an organic layer was concentrated, and the crude product thus obtained was separated by silica gel column chromatography (using a mixture solvent of hexane and toluene as a developing layer) to obtain Intermediate IM-2 (12.14 g, yield 66%).

FAB-MS was measured, mass number m/z=505 was observed as a molecular ion peak, and Intermediate IM-2 was identified.

(Synthesis of Compound A16)

Under an argon atmosphere, to a 500 ml, three-neck flask, 12.14 g (24.0 mmol) of Intermediate IM-2, 0.69 g (0.05 equiv, 1.2 mmol) of Pd(dba)₂, 2.30 g (1.0 equiv, 24.0 mmol) of NaO^{t}Bu, 240 mL of toluene, 4.06 g (1.0 equiv, 24.0 mmol) of di([1,1′-biphenyl]-2-yl)amine and 0.97 g (0.2 equiv, 4.8 mmol) of P^{t}Bu₃ were added in order, and stirred while heating and refluxing. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered, an organic layer was concentrated, and the crude product thus obtained was separated by silica gel column chromatography (using a mixture solvent of hexane and toluene as a developing layer) to obtain Compound A16 (12.12 g, yield 85%).

FAB-MS was measured, mass number m/z=594 was observed as a molecular ion peak, and Compound A16 was identified.

<Synthesis of Compound B19>

Amine Compound B19 according to an embodiment may be synthesized, for example, by the steps of Reaction 3 below.

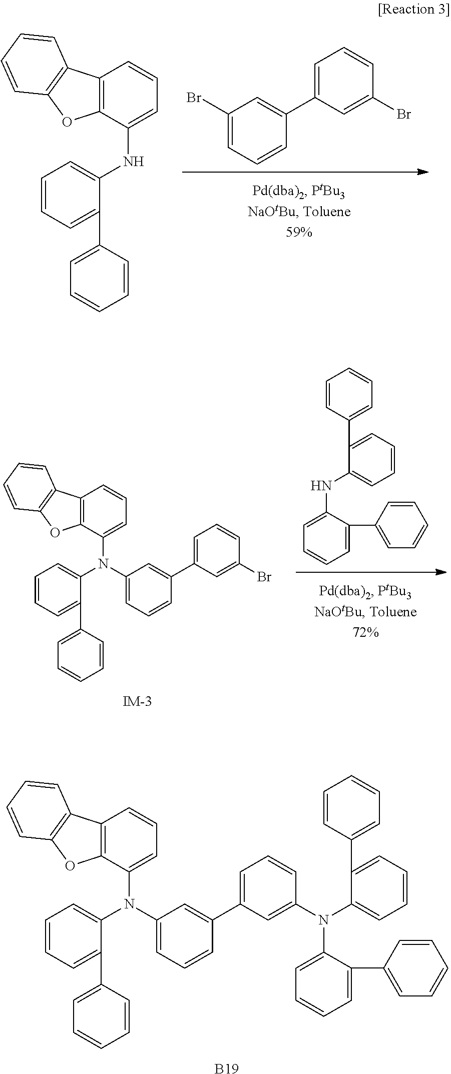

(Synthesis of Intermediate IM-3)

Under an argon atmosphere, to a 1,000 ml, three-neck flask, 10.0 g (29.8 mmol) of N-([1,1′-biphenyl]-2-yl)dibenzo[b,d]furan-4-amine, 0.86 g (0.05 equiv, 1.5 mmol) of Pd(dba)₂, 2.87 g (1.0 equiv, 29.8 mmol) of NaO^{t}Bu, 298 mL of toluene, 23.26 g (2.5 equiv, 74.5 mmol) of 3,3′-dibromo-1,1′-biphenyl and 1.21 g (0.2 equiv, 6.0 mmol) of P^{t}Bu₃ were added in order, and stirred while heating and refluxing. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered, an organic layer was concentrated, and the crude product thus obtained was separated by silica gel column chromatography (using a mixture solvent of hexane and toluene as a developing layer) to obtain Intermediate IM-3 (9.96 g, yield 59%).

FAB-MS was measured, mass number m/z=565 was observed as a molecular ion peak, and Intermediate IM-3 was identified.

(Synthesis of Compound B19)

Under an argon atmosphere, to a 500 ml, three-neck flask, 9.96 g (17.6 mmol) of Intermediate IM-3, 0.51 g (0.05 equiv, 0.88 mmol) of Pd(dba)₂, 1.69 g (1.0 equiv, 17.6 mmol) of NaO^{t}Bu, 176 mL of toluene, 5.65 g (1.0 equiv, 17.6 mmol) of di([1,1′-biphenyl]-2-yl)amine and 0.71 g (0.2 equiv, 3.5 mmol) of P^{t}Bu₃ were added in order, and stirred while heating and refluxing. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered, an organic layer was concentrated, and the crude product thus obtained was separated by silica gel column chromatography (using a mixture solvent of hexane and toluene as a developing layer) to obtain Compound B19 (10.22 g, yield 72%).

FAB-MS was measured, mass number m/z=806 was observed as a molecular ion peak, and Compound B19 was identified.

<Synthesis of Compound D27>

Amine Compound D27 according to an embodiment may be synthesized, for example, by the steps of Reaction 4 below.

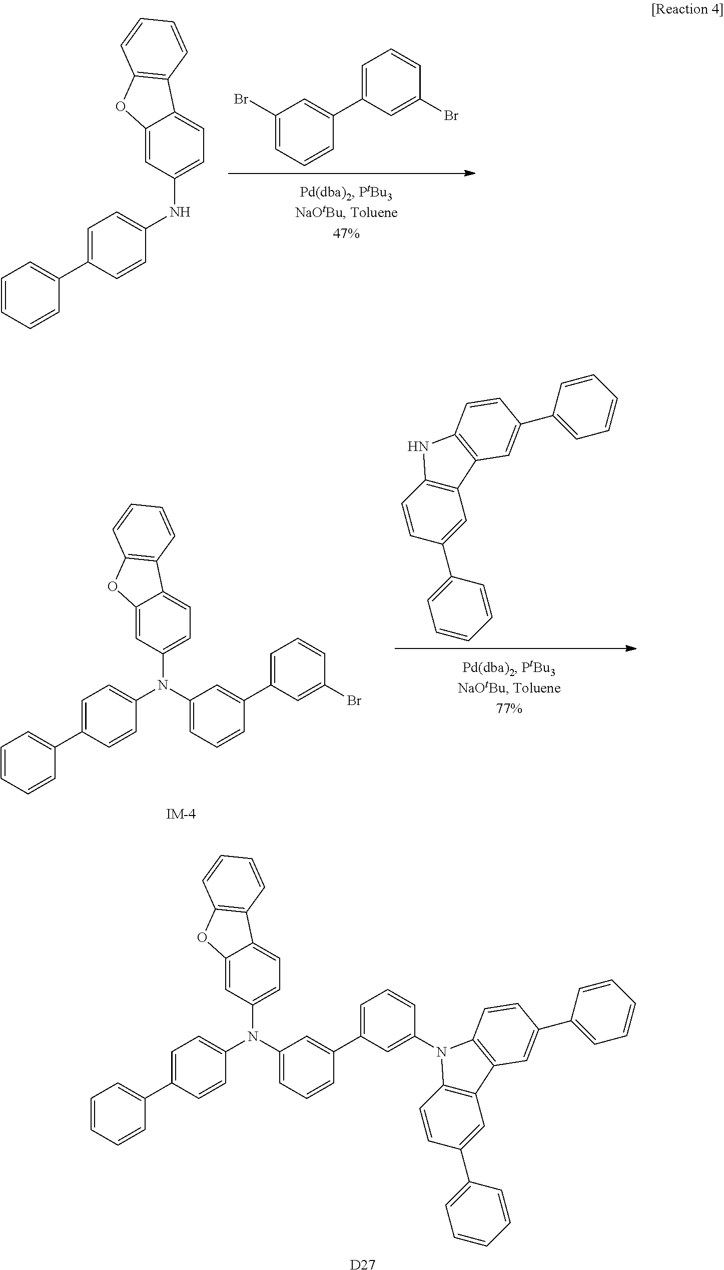

(Synthesis of Intermediate IM-4)

Under an argon atmosphere, to a 1,000 ml, three-neck flask, 9.00 g (26.8 mmol) of N-([1,1′-biphenyl]-4-yl)dibenzo[b,d]furan-3-amine, 0.77 g (0.05 equiv, 1.3 mmol) of Pd(dba)₂, 2.58 g (1.0 equiv, 26.8 mmol) of NaO^{t}Bu, 270 mL of toluene, 20.93 g (2.5 equiv, 67.08 mmol) of 3,3′-dibromo-1,1′-biphenyl and 1.09 g (0.2 equiv, 5.37 mmol) of P^{t}Bu₃ were added in order, and stirred while heating and refluxing. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered, an organic layer was concentrated, and the crude product thus obtained was separated by silica gel column chromatography (using a mixture solvent of hexane and toluene as a developing layer) to obtain Intermediate IM-4 (7.14 g, yield 47%).

FAB-MS was measured, mass number m/z=565 was observed as a molecular ion peak, and Intermediate IM-4 was identified.

(Synthesis of Compound D27)

Under an argon atmosphere, to a 300 ml, three-neck flask, 7.14 g (12.6 mmol) of Intermediate IM-4, 0.36 g (0.05 equiv, 0.63 mmol) of Pd(dba)₂, 1.21 g (1.0 equiv, 12.6 mmol) of NaO^{t}Bu, 130 mL of toluene, 4.03 g (1.0 equiv, 12.6 mmol) of 3,6-diphenyl-9H-carbazole and 0.51 g (0.2 equiv, 2.5 mmol) of P^{t}Bu₃ were added in order, and stirred while heating and refluxing. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered, an organic layer was concentrated, and the crude product thus obtained was separated by silica gel column chromatography (using a mixture solvent of hexane and toluene as a developing layer) to obtain Compound D27 (7.80 g, yield 77%).

FAB-MS was measured, mass number m/z=804 was observed as a molecular ion peak, and Compound D27 was identified.

<Synthesis of Compound I7>

Amine Compound I7 according to an embodiment may be synthesized, for example, by the steps of Reaction 5 below.

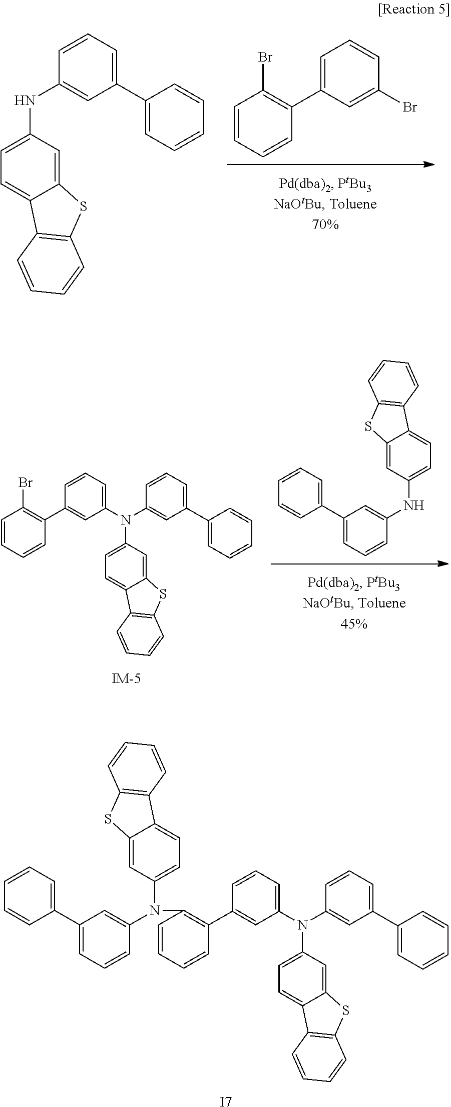

(Synthesis of Intermediate IM-5)

Under an argon atmosphere, to a 1,000 ml, three-neck flask, 10.0 g (28.5 mmol) of N-([1,1′-biphenyl]-3-yl)dibenzo[b,d]thiophen-3-amine, 0.82 g (0.05 equiv, 1.42 mmol) of Pd(dba)₂, 2.73 g (1.0 equiv, 28.5 mmol) of NaO^{t}Bu, 285 mL of toluene, 17.75 g (2.0 equiv, 56.9 mmol) of 2,3′-dibromo-1,1′-biphenyl and 1.15 g (0.2 equiv, 5.69 mmol) of P^{t}Bu₃ were added in order, and stirred while heating and refluxing. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered, an organic layer was concentrated, and the crude product thus obtained was separated by silica gel column chromatography (using a mixture solvent of hexane and toluene as a developing layer) to obtain Intermediate IM-5 (11.58 g, yield 70%).

FAB-MS was measured, mass number m/z=581 was observed as a molecular ion peak, and Intermediate IM-5 was identified.

(Synthesis of Compound I7)

Under an argon atmosphere, to a 500 ml, three-neck flask, 11.58 g (19.88 mmol) of Intermediate IM-5, 0.57 g (0.05 equiv, 0.99 mmol) of Pd(dba)₂, 1.91 g (1.0 equiv, 19.9 mmol) of NaO^{t}Bu, 200 mL of toluene, 10.48 g (1.5 equiv, 29.82 mmol) of N-([1,1′-biphenyl]-3-yl)dibenzo[b,d]thiophen-3-amine and 0.80 g (0.2 equiv, 4.0 mmol) of P^{t}Bu₃ were added in order, and stirred while heating and refluxing. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered, an organic layer was concentrated, and the crude product thus obtained was separated by silica gel column chromatography (using a mixture solvent of hexane and toluene as a developing layer) to obtain Compound I7 (7.60 g, yield 45%).

FAB-MS was measured, mass number m/z=852 was observed as a molecular ion peak, and Compound I7 was identified.

<Synthesis of Compound L30>

Amine Compound L30 according to an embodiment may be synthesized, for example, by the steps of Reaction 6 below.

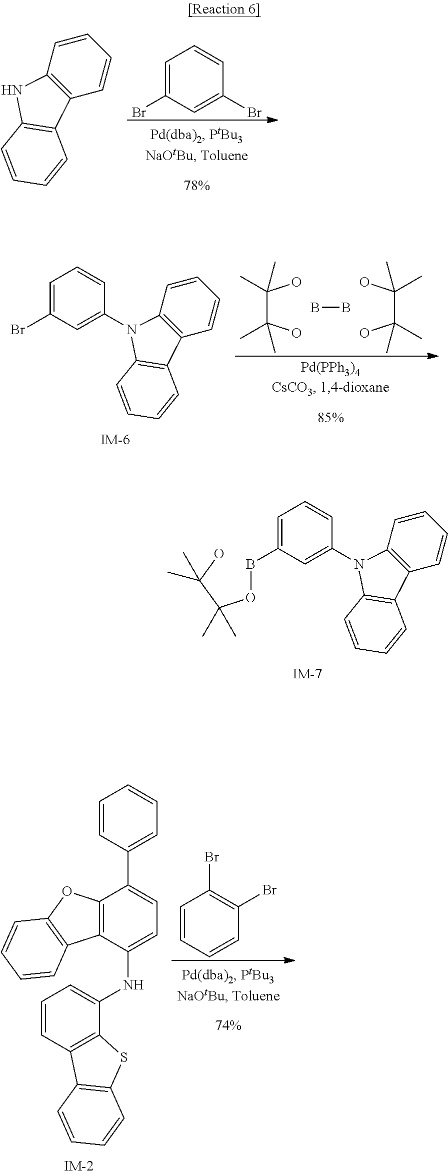

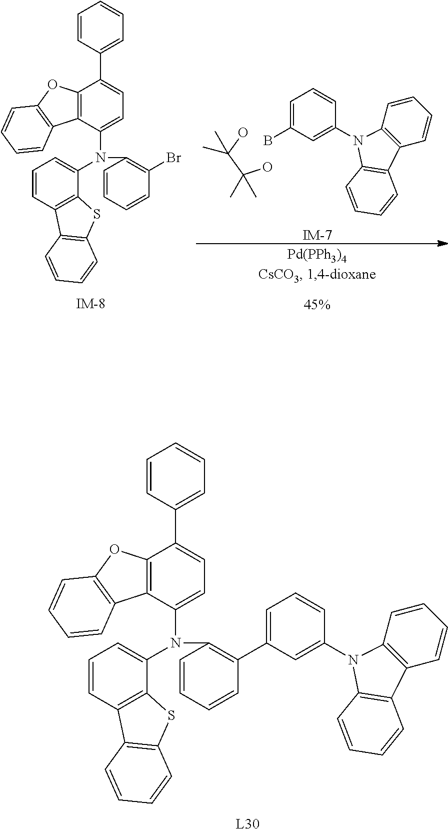

(Synthesis of Intermediate IM-6)

Under an argon atmosphere, to a 1,000 ml, three-neck flask, 8.00 g (47.8 mmol) of 9H-carbazole, 1.38 g (0.05 equiv, 2.39 mmol) of Pd(dba)₂, 4.60 g (1.0 equiv, 47.8 mmol) of NaO^{t}Bu, 480 mL of toluene, 28.22 g (2.5 equiv, 119.6 mmol) of 1,3-dibromobenzene and 1.94 g (0.2 equiv, 9.57 mmol) of P^{t}Bu₃ were added in order, and stirred while heating and refluxing. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered, an organic layer was concentrated, and the crude product thus obtained was separated by silica gel column chromatography (using a mixture solvent of hexane and toluene as a developing layer) to obtain Intermediate IM-6 (12.08 g, yield 78%).

FAB-MS was measured, mass number m/z=321 was observed as a molecular ion peak, and Intermediate IM-6 was identified.

(Synthesis of Intermediate IM-7)

Under an argon atmosphere, to a 1,000 ml, three-neck flask, 12.08 g (37.49 mmol) of Intermediate IM-6, 8.66 g (0.20 equiv, 7.50 mmol) of Pd(PPh₃)₄, 48.86 g (4.0 equiv, 150.0 mmol) of Cs₂CO₃, 370 mL of 1,4-dioxane, and 38.08 g (4.0 equiv, 150.0 mmol) of bis(pinacolato)diboron were added in order, and stirred while heating and refluxing. After cooling to room temperature, water and toluene were added to the reaction solution, and an organic layer was separately taken. An organic layer was washed with a saline solution and dried with MgSO₄. MgSO₄ was filtered, an organic layer was concentrated, and the crude product thus obtained was separated by silica gel column chromatography (using a mixture solvent of hexane and toluene as a developing layer) to obtain Intermediate IM-7 (11.70 g, yield 85%).

FAB-MS was measured, mass number m/z=369 was observed as a molecular ion peak, and Intermediate IM-7 was identified.

(Synthesis of Intermediate IM-8)

Under an argon atmosphere, to a 500 ml, three-neck flask, 9.00 g (20.4 mmol) of N-(dibenzo[b,d]thiophen-4-yl)-4-phenyldibenzo[b,d]furan-1-amine, 0.59 g (0.05 equiv, 1.02 mmol) of Pd(dba)₂, 1.96 g (1.0 equiv, 20.4 mmol) of NaO^{t}Bu, 200 mL of toluene, 12.02 g (2.5 equiv, 50.96 mmol) of 1,2-dibromobenzene and 0.82 g (0.2 equiv, 4.1 mmol) of P^{t}Bu₃ were added in order, and stirred while heating and refluxing. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered, an organic layer was concentrated, and the crude product thus obtained was separated by silica gel column chromatography (using a mixture solvent of hexane and toluene as a developing layer) to obtain Intermediate IM-8 (8.99 g, yield 74%).

FAB-MS was measured, mass number m/z=595 was observed as a molecular ion peak, and Intermediate IM-8 was identified.

(Synthesis of Compound L30)

Under an argon atmosphere, to a 500 ml, three-neck flask, 8.99 g (15.1 mmol) of Intermediate IM-8, 3.48 g (0.20 equiv, 3.01 mmol) of Pd(PPh₃)₄, 9.82 g (2.0 equiv, 30.1 mmol) of Cs₂CO₃, 150 mL of 1,4-dioxane, and 5.56 g (1.0 equiv, 15.1 mmol) of Intermediate IM-7 were added in order, and stirred while heating and refluxing. After cooling to room temperature, water and toluene are added to the reaction solution, and an organic layer was separately taken. The organic layer was washed with a saline solution and dried with MgSO₄. MgSO₄ was filtered, an organic layer was concentrated, and the crude product thus obtained was separated by silica gel column chromatography (using a mixture solvent of hexane and toluene as a developing layer) to obtain Compound L30 (5.12 g, yield 45%).

FAB-MS was measured, mass number m/z=758 was observed as a molecular ion peak, and Compound L30 was identified.

<Synthesis of Compound Q14>

Amine Compound Q14 according to an embodiment may be synthesized, for example, by the steps of Reaction 7 below.

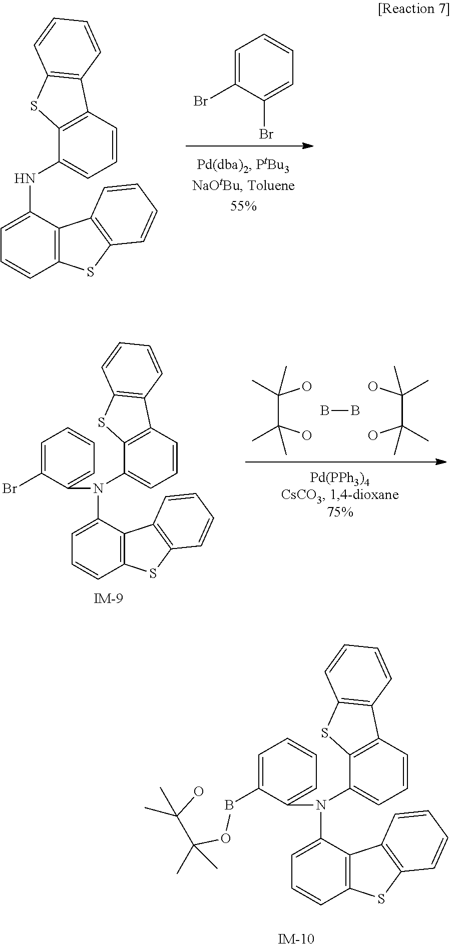

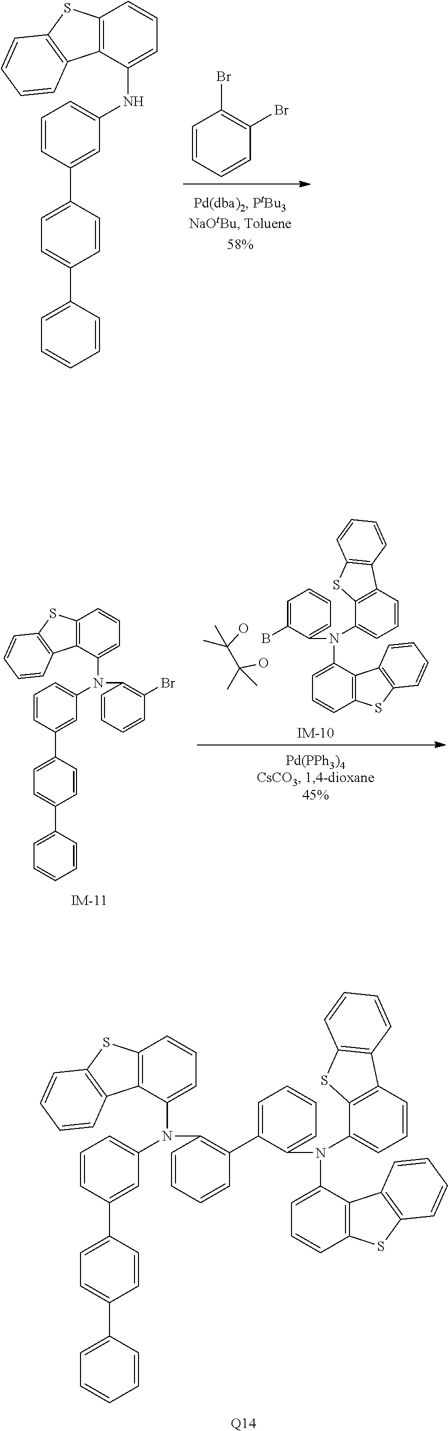

(Synthesis of Intermediate IM-9)

Under an argon atmosphere, to a 1,000 ml, three-neck flask, 15.00 g (39.32 mmol) of N-(dibenzo[b,d]thiophen-4-yl)dibenzo[b,d]thiophen-1-amine, 1.13 g (0.05 equiv, 1.97 mmol) of Pd(dba)₂, 3.78 g (1.0 equiv, 39.3 mmol) of NaO^{t}Bu, 400 mL of toluene, 23.19 g (2.5 equiv, 98.29 mmol) of 1,2-dibromobenzene and 1.59 g (0.2 equiv, 7.86 mmol) of P^{t}Bu₃ were added in order, and stirred while heating and refluxing. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered, an organic layer was concentrated, and the crude product thus obtained was separated by silica gel column chromatography (using a mixture solvent of hexane and toluene as a developing layer) to obtain Intermediate IM-9 (11.60 g, yield 55%).

FAB-MS was measured, mass number m/z=535 was observed as a molecular ion peak, and Intermediate IM-9 was identified.

(Synthesis of Intermediate IM-10)

Under an argon atmosphere, to a 1,000 ml, three-neck flask, 11.00 g (20.43 mmol) of Intermediate IM-9, 4.72 g (0.20 equiv, 4.09 mmol) of Pd(PPh₃)₄, 26.62 g (4.0 equiv, 81.71 mmol) of Cs₂CO₃, 200 mL of 1,4-dioxane, and 20.75 g (4.0 equiv, 81.71 mmol) of bis(pinacolato)diboron were added in order, and stirred while heating and refluxing. After cooling to room temperature, water and toluene were added to the reaction solution, and an organic layer was separately taken. The organic layer was washed with a saline solution and dried with MgSO₄. MgSO₄ was filtered, an organic layer was concentrated, and the crude product thus obtained was separated by silica gel column chromatography (using a mixture solvent of hexane and toluene as a developing layer) to obtain Intermediate IM-10 (8.90 g, yield 75%).

FAB-MS was measured, mass number m/z=583 was observed as a molecular ion peak, and Intermediate IM-10 was identified.

(Synthesis of Intermediate IM-11)

Under an argon atmosphere, to a 500 ml, three-neck flask, 10.00 g (23.39 mmol) of N-([1,1′:4′,1″-terphenyl]-3-yl)dibenzo[b,d]thiophen-1-amine, 0.67 g (0.05 equiv, 1.17 mmol) of Pd(dba)₂, 2.25 g (1.0 equiv, 23.4 mmol) of NaO^{t}Bu, 230 mL of toluene, 13.79 g (2.5 equiv, 58.47 mmol) of 1,2-dibromobenzene and 0.95 g (0.2 equiv, 4.68 mmol) of P^{t}Bu₃ were added in order, and stirred while heating and refluxing. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered, an organic layer was concentrated, and the crude product thus obtained was separated by silica gel column chromatography (using a mixture solvent of hexane and toluene as a developing layer) to obtain Intermediate IM-11 (7.91 g, yield 58%).

FAB-MS was measured, mass number m/z=581 was observed as a molecular ion peak, and Intermediate IM-11 was identified.

(Synthesis of Compound Q14)

Under an argon atmosphere, to a 200 ml, three-neck flask, 5.00 g (8.58 mmol) of Intermediate IM-11, 1.98 g (0.20 equiv, 1.72 mmol) of Pd(PPh₃)₄, 5.59 g (2.0 equiv, 17.2 mmol) of Cs₂CO₃, 85 mL of 1,4-dioxane, and 5.01 g (1.0 equiv, 8.58 mmol) of Intermediate IM-10 were added in order, and stirred while heating and refluxing. After cooling to room temperature, water and toluene were added to the reaction solution, and an organic layer was separately taken. The organic layer was washed with a saline solution and dried with MgSO₄. MgSO₄ was filtered, an organic layer was concentrated, and the crude product thus obtained was separated by silica gel column chromatography (using a mixture solvent of hexane and toluene as a developing layer) to obtain Compound Q14 (3.7 g, yield 45%).

FAB-MS was measured, mass number m/z=958 was observed as a molecular ion peak, and Compound Q14 was identified.

<Synthesis of Compound Q30>

Amine Compound Q30 according to an embodiment may be synthesized, for example, by the steps of Reaction 8 below.

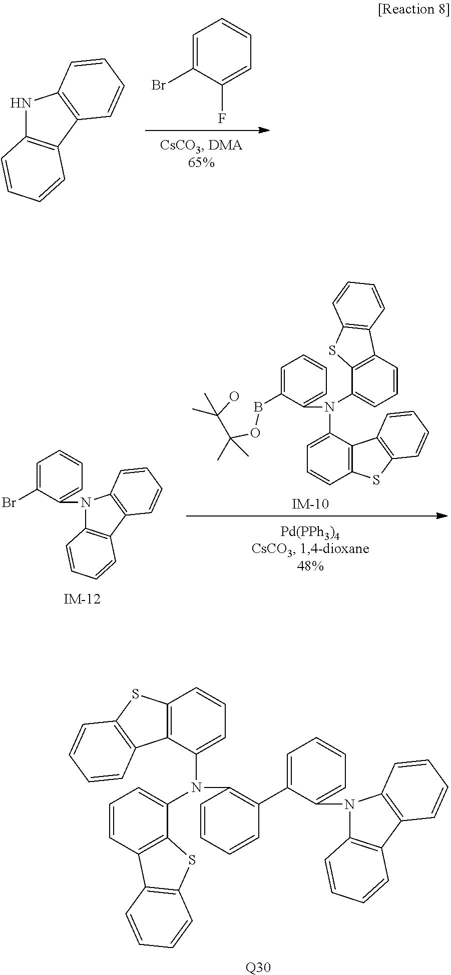

(Synthesis of Intermediate IM-12)

Under an argon atmosphere, to a 200 ml, three-neck flask, 10.00 g (59.81 mmol) of 9H-carbazole, 23.07 g (2.0 equiv, 119.6 mmol) of Cs₂CO₃, 60 mL of DMA, and 20.93 g (2.0 equiv, 119.6 mmol) of 1-bromo-2-fluorobenzene were added in order, and heated to 120° C. and stirred. After cooling to room temperature, water and toluene were added to the reaction solution, and an organic layer was separately taken. The organic layer was washed with a saline solution and dried with MgSO₄. MgSO₄ was filtered, an organic layer was concentrated, and the crude product thus obtained was separated by silica gel column chromatography (using a mixture solvent of hexane and toluene as a developing layer) to obtain Intermediate IM-12 (12.51 g, yield 65%).

FAB-MS was measured, mass number m/z=321 was observed as a molecular ion peak, and Intermediate IM-12 was identified.

(Synthesis of Compound Q30)

Under an argon atmosphere, to a 200 ml, three-neck flask, 2.00 g (6.21 mmol) of Intermediate IM-12, 1.43 g (0.20 equiv, 1.24 mmol) of Pd(PPh₃)₄, 4.04 g (2.0 equiv, 12.4 mmol) of Cs₂CO₃, 60 mL of 1,4-dioxane, and 3.62 g (1.0 equiv, 6.21 mmol) of Intermediate IM-10 were added in order, and stirred while heating and refluxing. After cooling to room temperature, water and toluene were added to the reaction solution, and an organic layer was separately taken. The organic layer was washed with a saline solution and dried with MgSO₄. MgSO₄ was filtered, an organic layer was concentrated, and the crude product thus obtained was separated by silica gel column chromatography (using a mixture solvent of hexane and toluene as a developing layer) to obtain Compound Q30 (2.1 g, yield 48%).

FAB-MS was measured, mass number m/z=698 was observed as a molecular ion peak, and Compound Q30 was identified.

<Synthesis of Compound T13>

Amine Compound T13 according to an embodiment may be synthesized, for example, by the steps of Reaction 9 below.

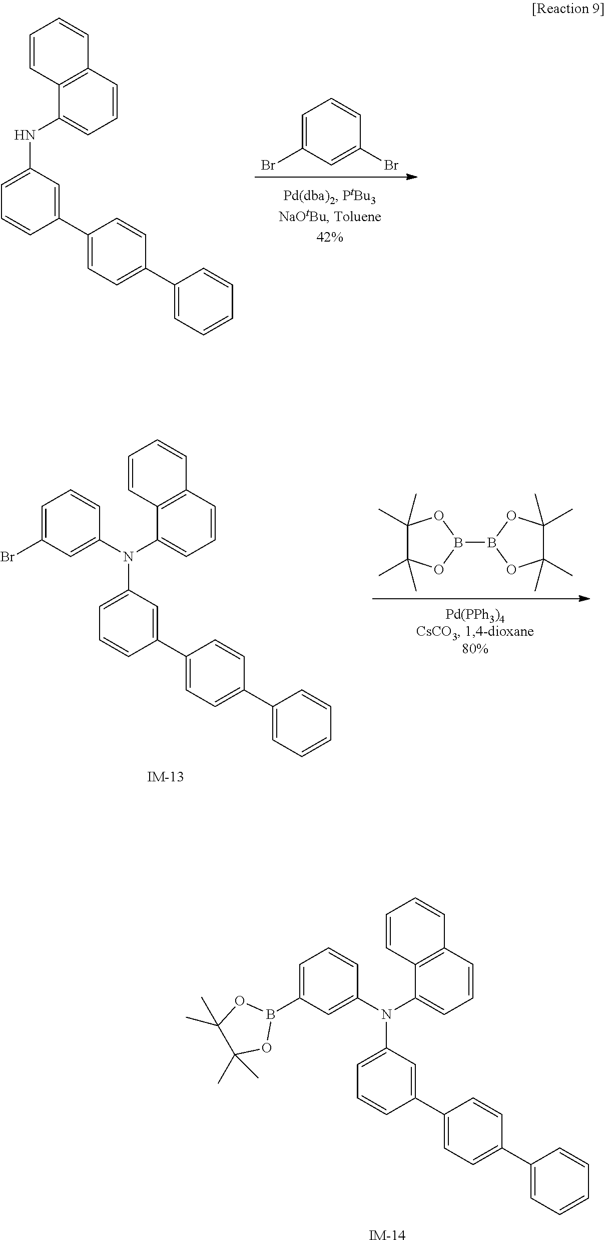

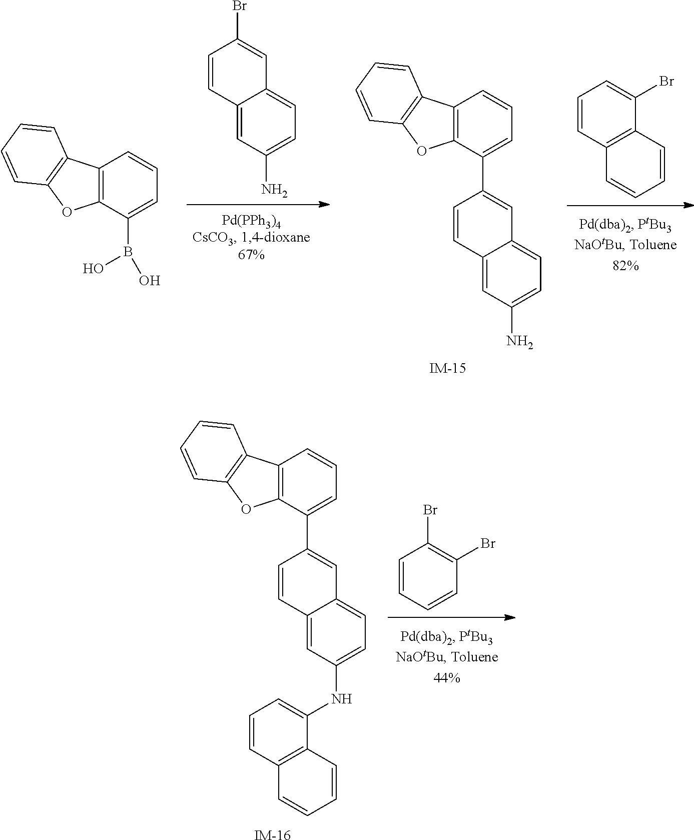

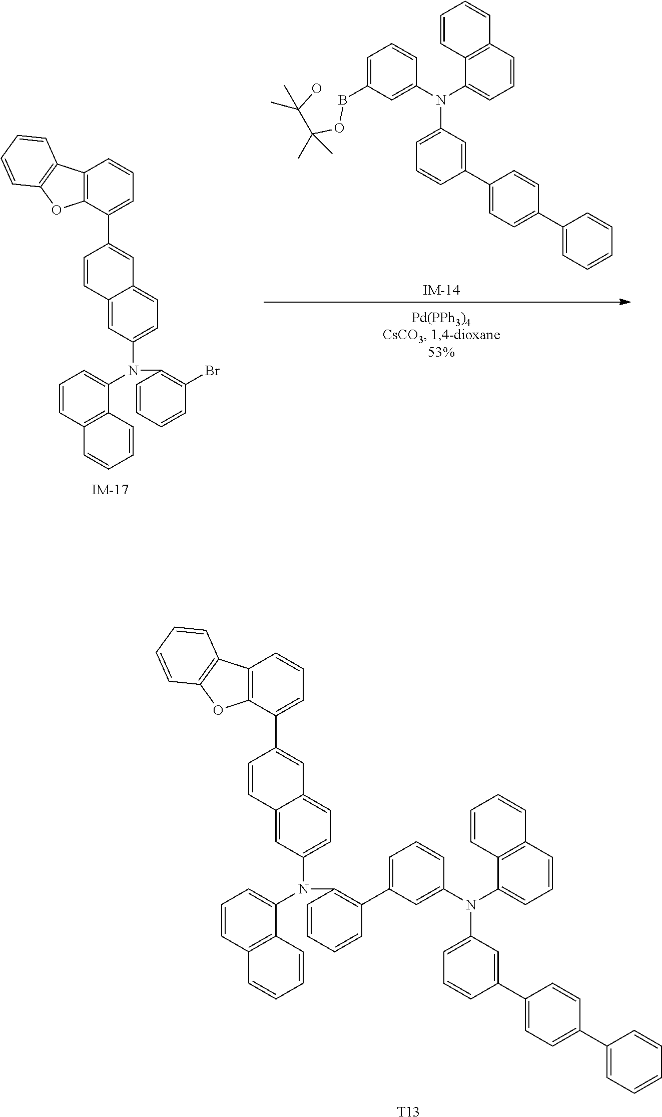

(Synthesis of Intermediate IM-13)

Under an argon atmosphere, to a 1,000 ml, three-neck flask, 10.00 g (26.92 mmol) of N-([1,1′:4′,1″-terphenyl]-3-yl)naphthalen-1-amine, 0.77 g (0.05 equiv, 1.4 mmol) of Pd(dba)₂, 2.59 g (1.0 equiv, 26.9 mmol) of NaO^{t}Bu, 270 mL of toluene, 15.88 g (2.5 equiv, 67.30 mmol) of 1,3-dibromobenzene and 1.94 g (0.2 equiv, 9.57 mmol) of P^{t}Bu₃ were added in order, and stirred while heating and refluxing. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered, an organic layer was concentrated, and the crude product thus obtained was separated by silica gel column chromatography (using a mixture solvent of hexane and toluene as a developing layer) to obtain Intermediate IM-13 (5.99 g, yield 42%).

FAB-MS was measured, mass number m/z=525 was observed as a molecular ion peak, and Intermediate IM-13 was identified.

(Synthesis of Intermediate IM-14)

Under an argon atmosphere, to a 300 ml, three-neck flask, 5.99 g (11.4 mmol) of Intermediate IM-13, 2.63 g (0.20 equiv, 2.28 mmol) of Pd(PPh₃)₄, 11.12 g (3.0 equiv, 34.13 mmol) of Cs₂CO₃, 110 mL of 1,4-dioxane, and 8.67 g (3.0 equiv, 34.1 mmol) of bis(pinacolato)diboron were added in order, and stirred while heating and refluxing. After cooling to room temperature, water and toluene were added to the reaction solution, and an organic layer was separately taken. The organic layer was washed with a saline solution and dried with MgSO₄. MgSO₄ was filtered, an organic layer was concentrated, and the crude product thus obtained was separated by silica gel column chromatography (using a mixture solvent of hexane and toluene as a developing layer) to obtain Intermediate IM-14 (5.23 g, yield 80%).

FAB-MS was measured, mass number m/z=573 was observed as a molecular ion peak, and Intermediate IM-14 was identified.

(Synthesis of Intermediate IM-15)

Under an argon atmosphere, to a 2,000 ml, three-neck flask, 15.00 g (70.75 mmol) of dibenzo[b,d]furan-4-ylboronic acid, 16.35 g (0.20 equiv, 14.15 mmol) of Pd(PPh₃)₄, 23.05 g (1.0 equiv, 70.75 mmol) of Cs₂CO₃, 700 mL of 1,4-dioxane, and 15.71 g (1.0 equiv, 70.75 mmol) of 6-bromonaphthalen-2-amine were added in order, and stirred while heating and refluxing. After cooling to room temperature, water and toluene were added to the reaction solution, and an organic layer was separately taken. The organic layer was washed with a saline solution and dried with MgSO₄. MgSO₄ was filtered, an organic layer was concentrated, and the crude product thus obtained was separated by silica gel column chromatography (using a mixture solvent of hexane and toluene as a developing layer) to obtain Intermediate IM-15 (14.67 g, yield 67%).

FAB-MS was measured, mass number m/z=309 was observed as a molecular ion peak, and Intermediate IM-15 was identified.

(Synthesis of Intermediate IM-16)

Under an argon atmosphere, to a 2,000 ml, three-neck flask, 14.67 g (47.42 mmol) of Intermediate IM-15, 1.36 g (0.05 equiv, 2.37 mmol) of Pd(dba)₂, 4.56 g (1.0 equiv, 47.4 mmol) of NaO^{t}Bu, 470 mL of toluene, 9.82 g (1.0 equiv, 47.4 mmol) of 1-bromonaphthalene and 1.92 g (0.2 equiv, 9.48 mmol) of P^{t}Bu₃ were added in order, and stirred while heating and refluxing. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered, an organic layer was concentrated, and the crude product thus obtained was separated by silica gel column chromatography (using a mixture solvent of hexane and toluene as a developing layer) to obtain Intermediate IM-16 (16.90 g, yield 82%).

FAB-MS was measured, mass number m/z=435 was observed as a molecular ion peak, and Intermediate IM-16 was identified.

(Synthesis of Intermediate IM-17)

Under an argon atmosphere, to a 1,000 ml, three-neck flask, 16.90 g (38.80 mmol) of Intermediate IM-16, 1.12 g (0.05 equiv, 1.94 mmol) of Pd(dba)₂, 3.73 g (1.0 equiv, 38.8 mmol) of NaO^{t}Bu, 390 mL of toluene, 22.89 g (2.5 equiv, 97.01 mmol) of 1,2-dibromobenzene and 1.57 g (0.2 equiv, 7.76 mmol) of P^{t}Bu₃ were added in order, and stirred while heating and refluxing. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered, an organic layer was concentrated, and the crude product thus obtained was separated by silica gel column chromatography (using a mixture solvent of hexane and toluene as a developing layer) to obtain Intermediate IM-17 (10.05 g, yield 44%).

FAB-MS was measured, mass number m/z=589 was observed as a molecular ion peak, and Intermediate IM-17 was identified.

(Synthesis of Compound T13)

Under an argon atmosphere, to a 300 ml, three-neck flask, 5.00 g (8.47 mmol) of Intermediate IM-17, 1.96 g (0.20 equiv, 1.70 mmol) of Pd(PPh₃)₄, 5.51 g (2.0 equiv, 16.9 mmol) of Cs₂CO₃, 80 mL of 1,4-dioxane, and 4.86 g (1.0 equiv, 8.47 mmol) of Intermediate IM-14 were added in order, and stirred while heating and refluxing. After cooling to room temperature, water and toluene were added to the reaction solution, and an organic layer was separately taken. The organic layer was washed with a saline solution and dried with MgSO₄. MgSO₄ was filtered, an organic layer was concentrated, and the crude product thus obtained was separated by silica gel column chromatography (using a mixture solvent of hexane and toluene as a developing layer) to obtain Compound T13 (4.3 g, yield 53%).

FAB-MS was measured, mass number m/z=956 was observed as a molecular ion peak, and Compound T13 was identified.

<Synthesis of Compound X2>

Amine Compound X2 according to an embodiment may be synthesized, for example, by the steps of Reaction 10 below.

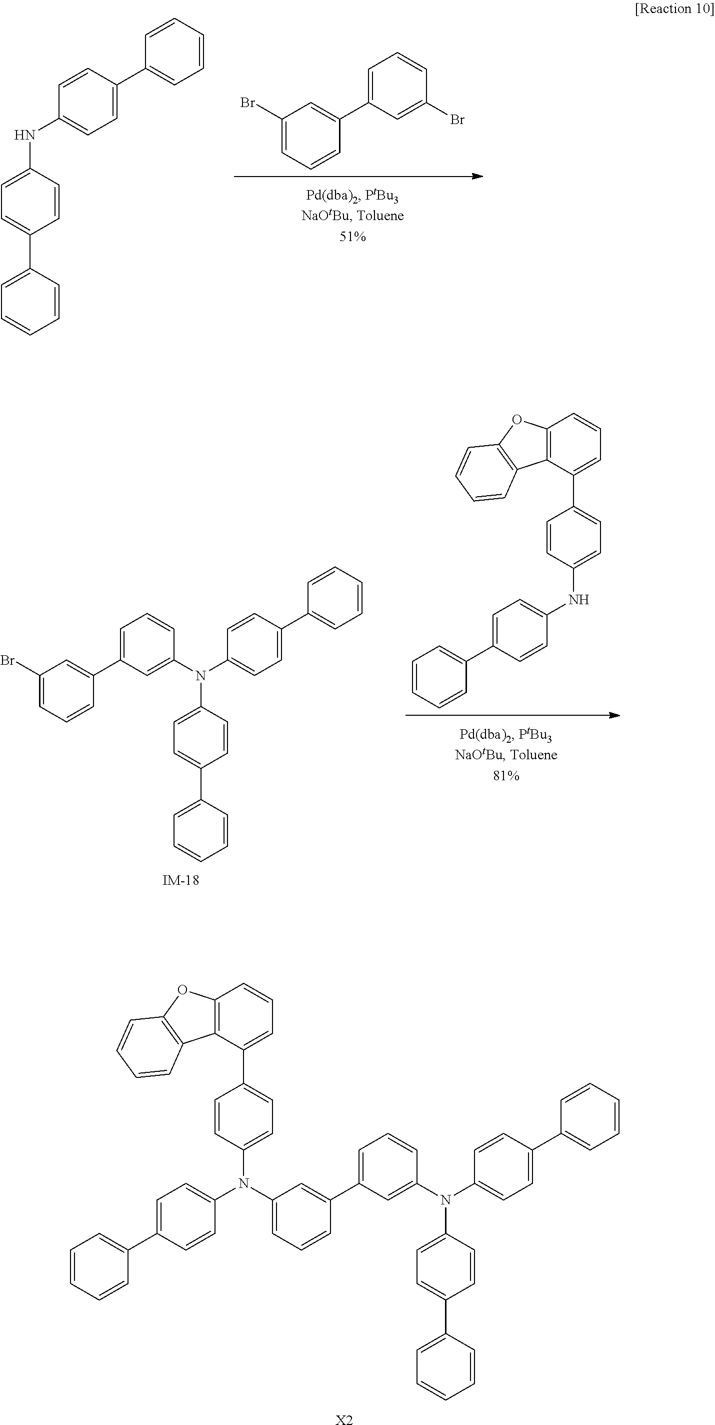

(Synthesis of Intermediate IM-18)

Under an argon atmosphere, to a 1,000 ml, three-neck flask, 10.0 g (31.1 mmol) of di([1,1′-biphenyl]-4-yl)amine, 0.89 g (0.05 equiv, 1.56 mmol) of Pd(dba)₂, 2.99 g (1.0 equiv, 31.1 mmol) of NaO^{t}Bu, 310 mL of toluene, 24.27 g (2.5 equiv, 77.78 mmol) of 3,3′-dibromo-1,1′-biphenyl and 1.26 g (0.2 equiv, 6.22 mmol) of P^{t}Bu₃ were added in order, and stirred while heating and refluxing. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered, an organic layer was concentrated, and the crude product thus obtained was separated by silica gel column chromatography (using a mixture solvent of hexane and toluene as a developing layer) to obtain Intermediate IM-18 (8.78 g, yield 51%).

FAB-MS was measured, mass number m/z=551 was observed as a molecular ion peak, and Intermediate IM-18 was identified.

(Synthesis of Compound X2)

Under an argon atmosphere, to a 500 ml, three-neck flask, 8.78 g (15.9 mmol) of Intermediate IM-18, 0.46 g (0.05 equiv, 0.79 mmol) of Pd(dba)₂, 1.53 g (1.0 equiv, 15.9 mmol) of NaO^{t}Bu, 160 mL of toluene, 6.54 g (1.0 equiv, 15.9 mmol) of N-(4-(dibenzo[b,d]furan-1-yl)phenyl)-[1,1′-biphenyl]-4-amine and 0.64 g (0.2 equiv, 3.2 mmol) of P^{t}Bu₃ were added in order, and stirred while heating and refluxing. After cooling to room temperature, water was added to the reaction solution, and an organic layer was separately taken. Toluene was added to an aqueous layer, and an organic layer was further extracted. Organic layers were collected, washed with a saline solution, and dried with MgSO₄. MgSO₄ was filtered, an organic layer was concentrated, and the crude product thus obtained was separated by silica gel column chromatography (using a mixture solvent of hexane and toluene as a developing layer) to obtain Compound X2 (11.33 g, yield 81%).

FAB-MS was measured, mass number m/z=882 was observed as a molecular ion peak, and Compound X2 was identified.

2. Manufacture and Evaluation of Light Emitting Diode

(Manufacture of Light Emitting Diode)

A light emitting diode of an embodiment including an amine compound of an embodiment in a hole transport layer was manufactured by a method below. Light emitting diodes of Examples 1 to 10 were manufactured using amine compounds of Compounds A11, A16, B19, D27, I7, L30, Q14, Q30, T13, B149, and X2 as materials for a hole transport layer. In Comparative Examples 1 to 28, light emitting diodes were manufactured using Comparative Compounds R-1 to R-28 below as materials of a hole transport layer.

The compounds used in hole transport layer in Example 1 to Example 10, and Comparative Example 1 to Comparative Example 28 are shown below.

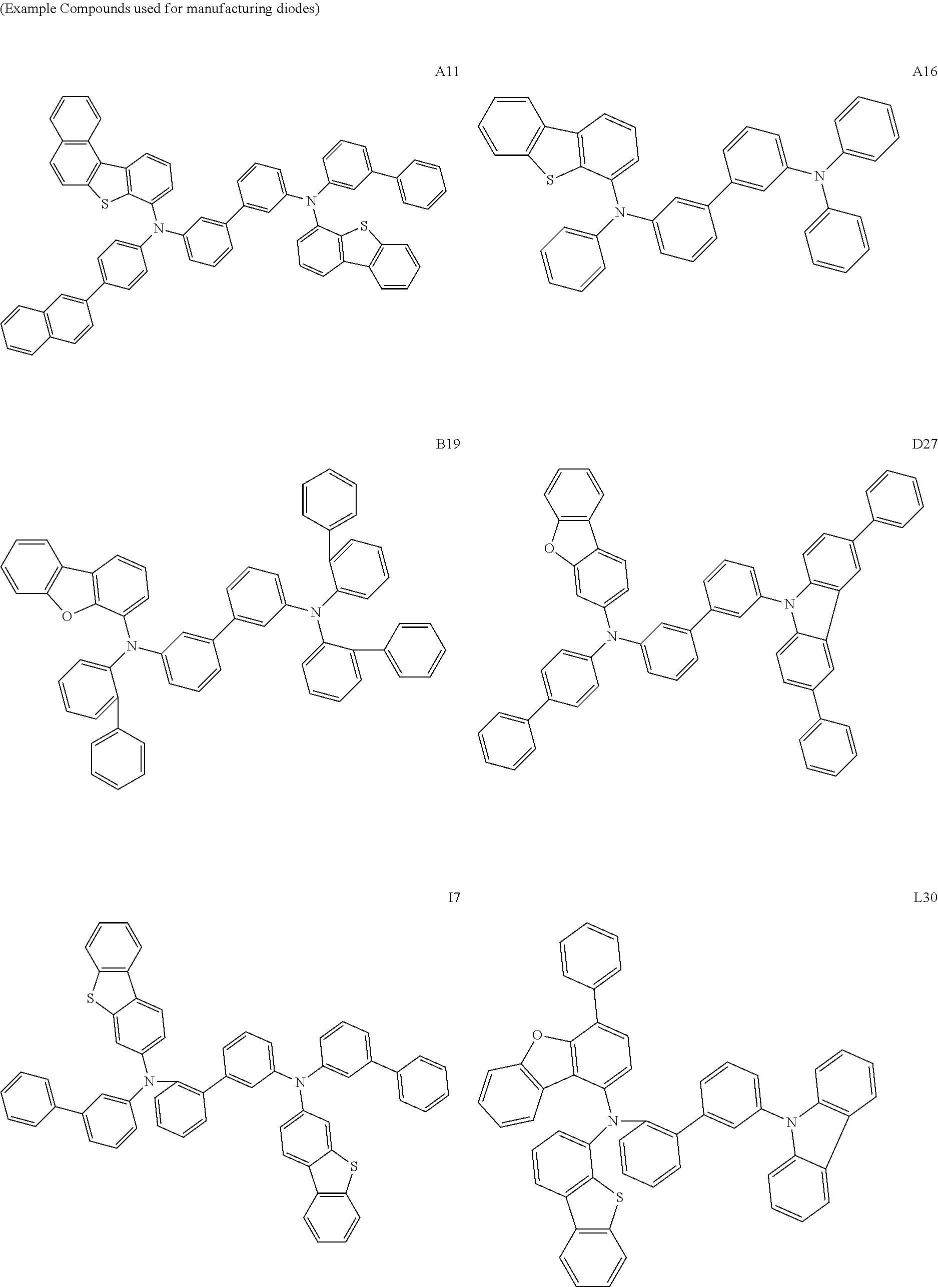

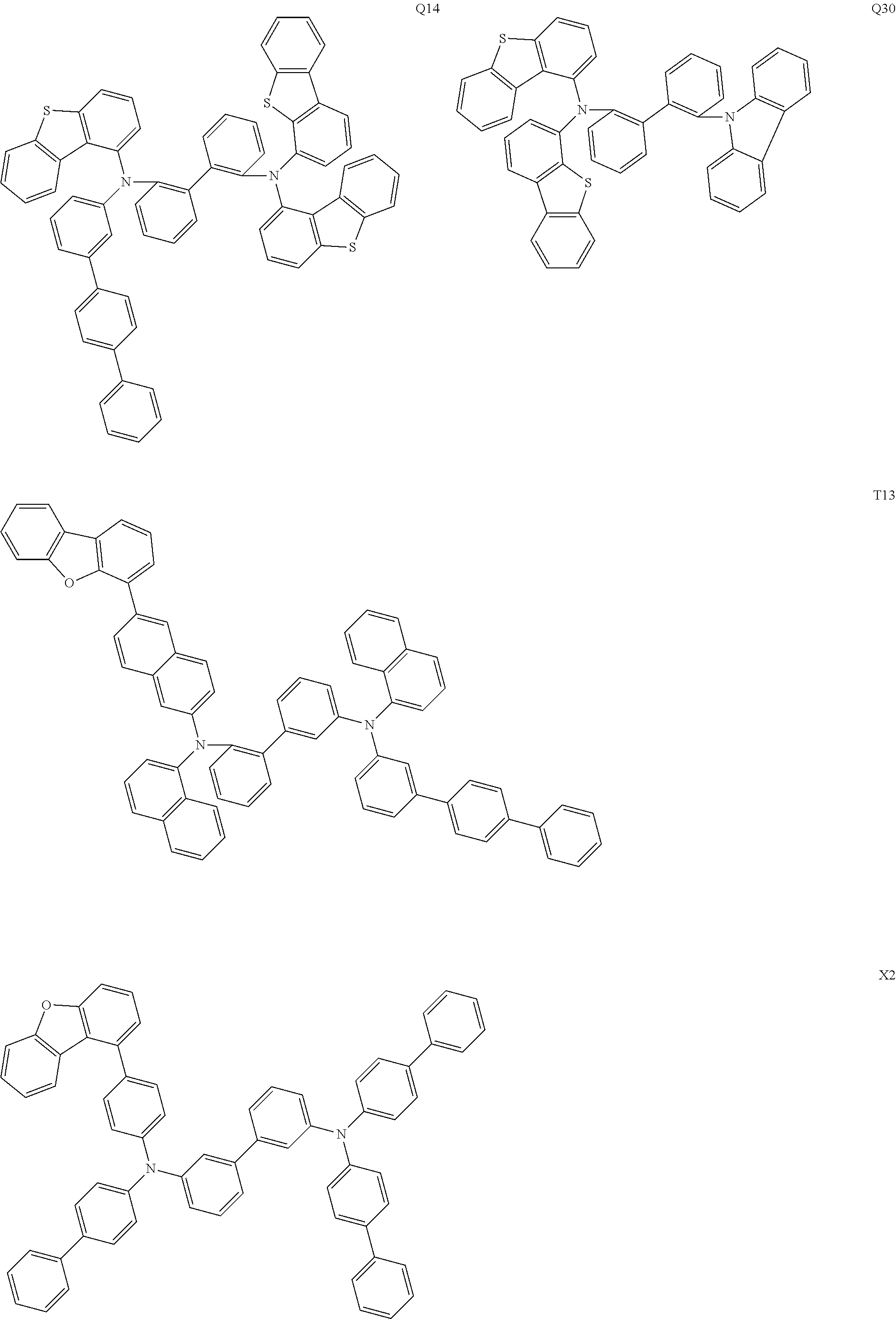 [SMILES: c1ccc(-c2ccc(-c3cccc(N(c4ccccc4-c4ccccc4N(c4cccc5c4sc4ccccc45)c4cccc5sc6ccccc6c45)c4cccc5sc6ccccc6c45)c3)cc2)cc1 ; c1ccc(N(c2cccc3c2sc2ccccc23)c2cccc3sc4ccccc4c23)c(-c2ccccc2-n2c3ccccc3c3ccccc32)c1 ; c1ccc(-c2ccc(-c3cccc(N(c4cccc(-c5ccccc5N(c5ccc6cc(-c7cccc8c7oc7ccccc78)ccc6c5)c5cccc6ccccc56)c4)c4cccc5ccccc45)c3)cc2)cc1 ; c1ccc(-c2ccc(N(c3ccc(-c4ccccc4)cc3)c3cccc(-c4cccc(N(c5ccc(-c6ccccc6)cc5)c5ccc(-c6cccc7oc8ccccc8c67)cc5)c4)c3)cc2)cc1]

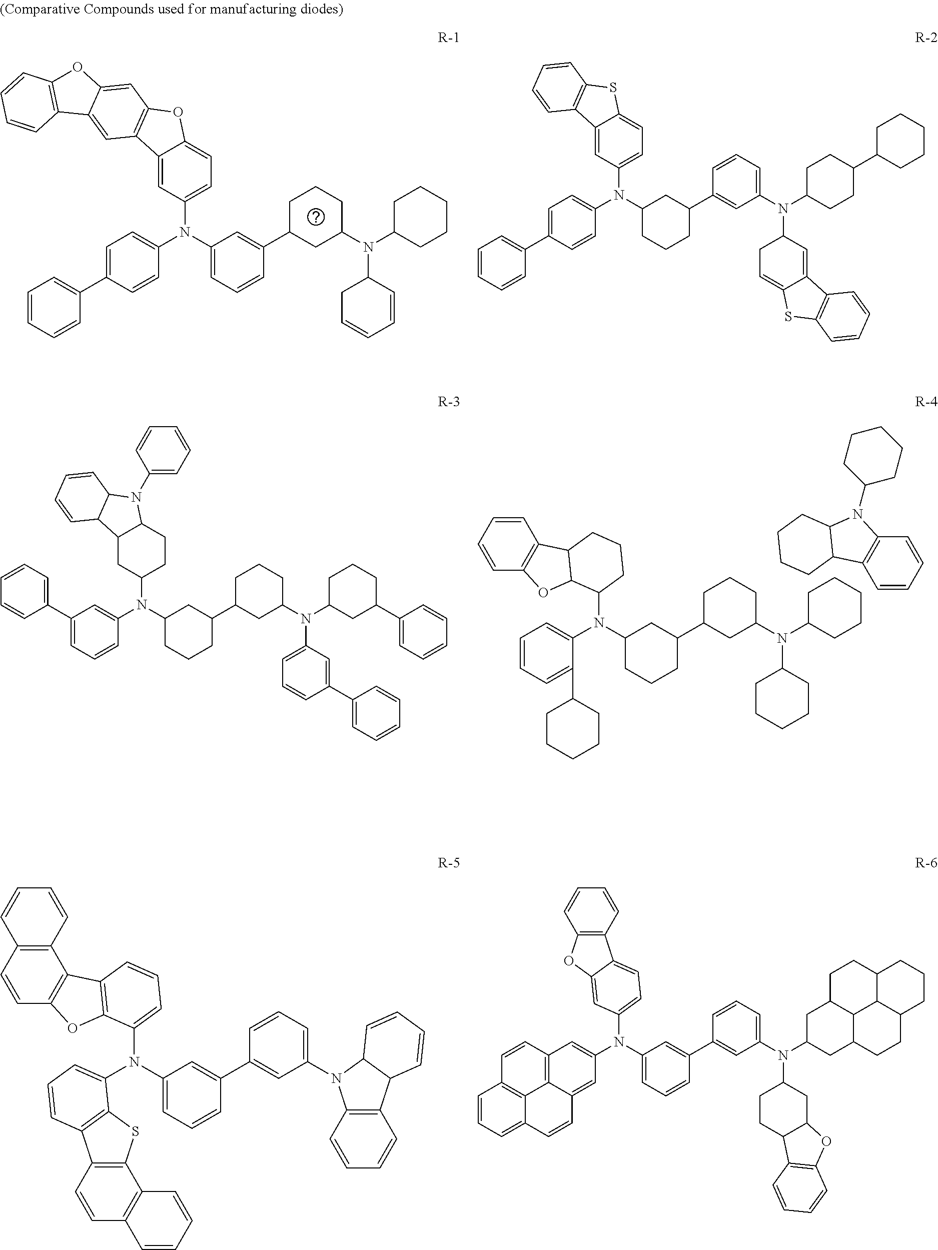

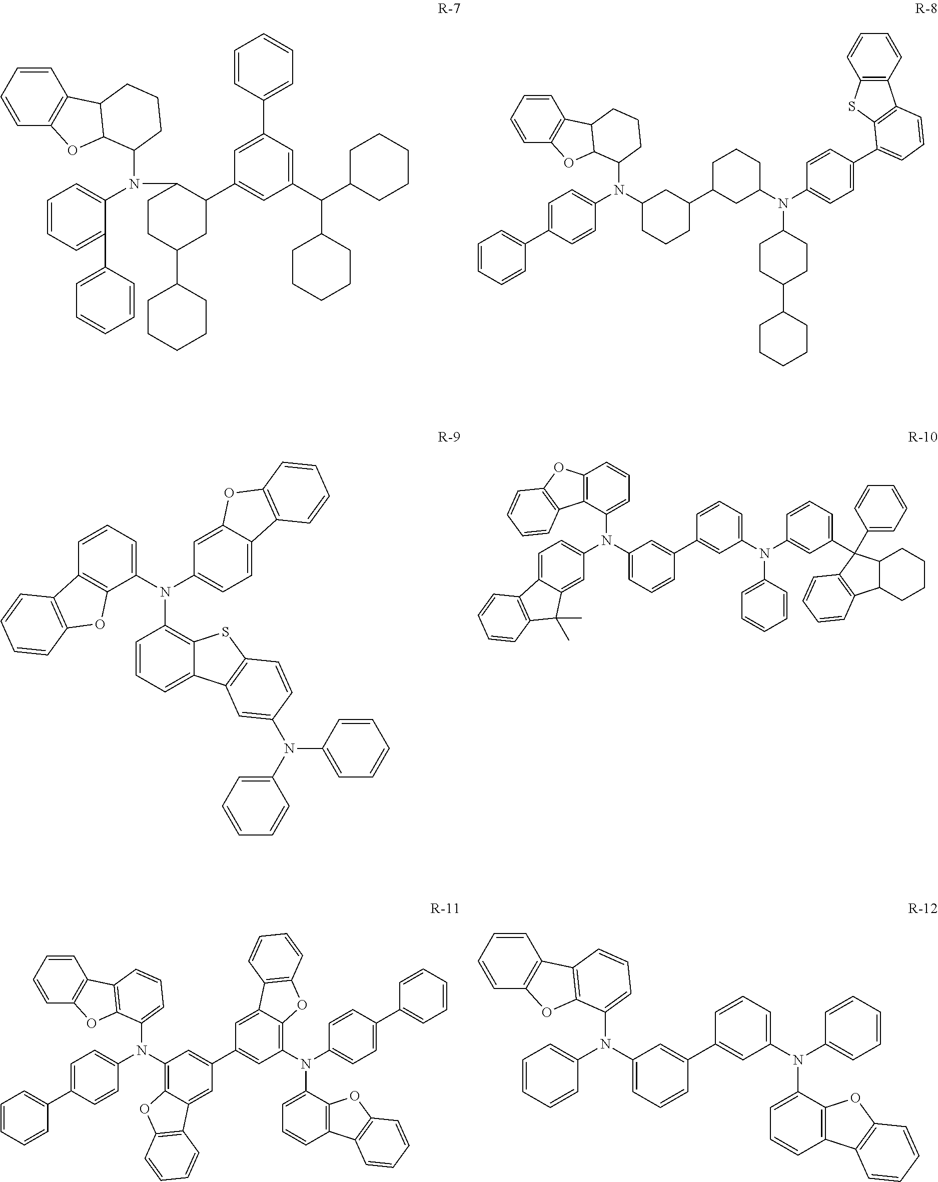

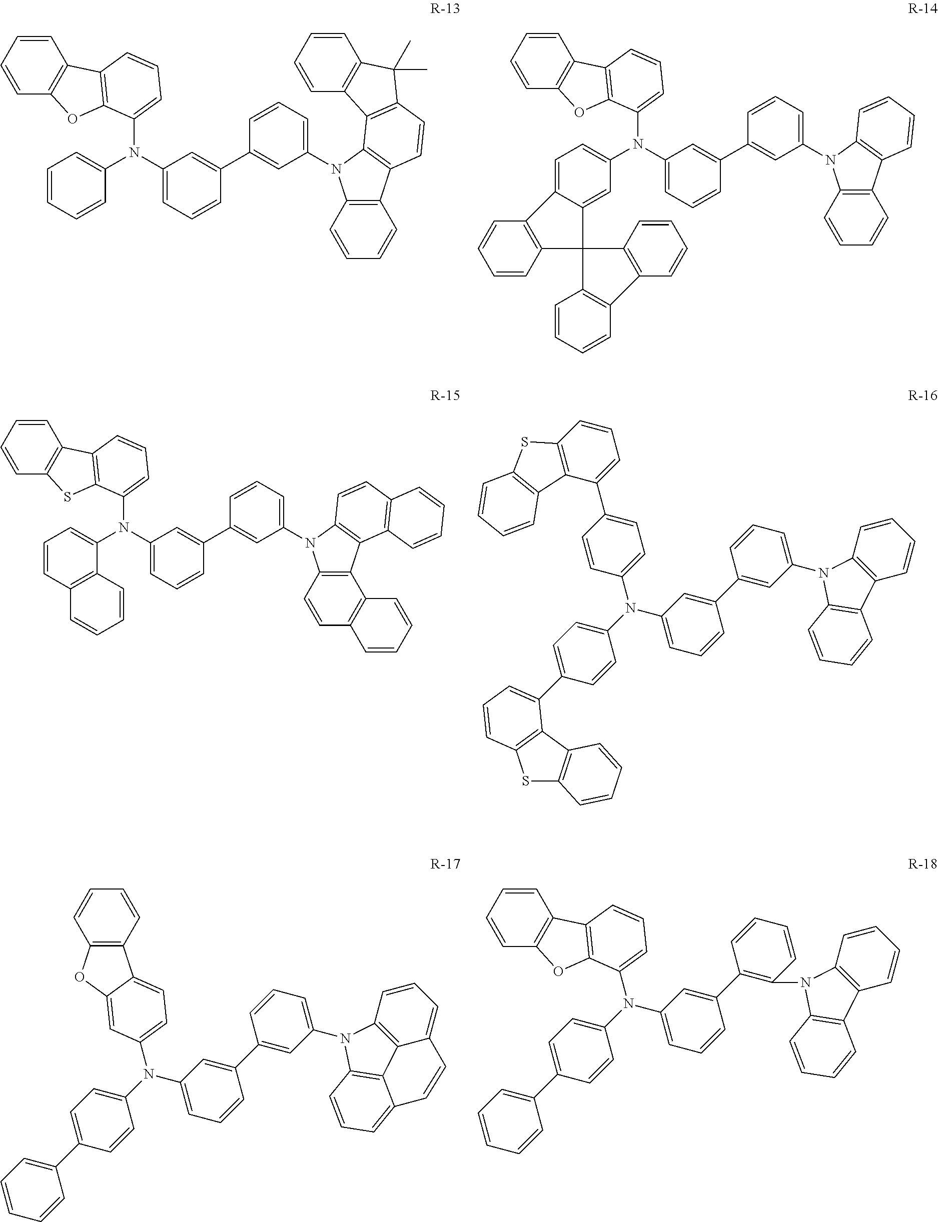

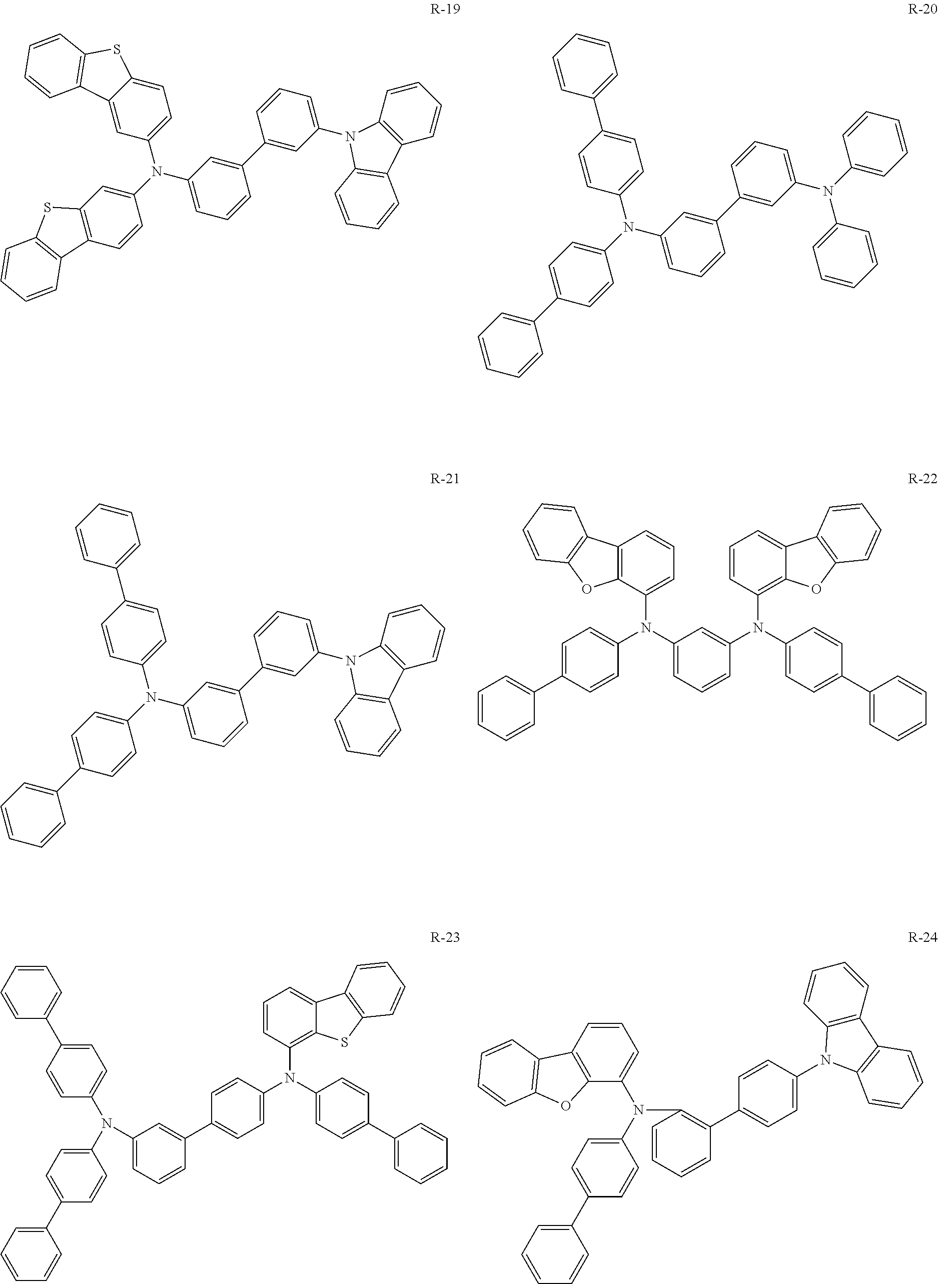

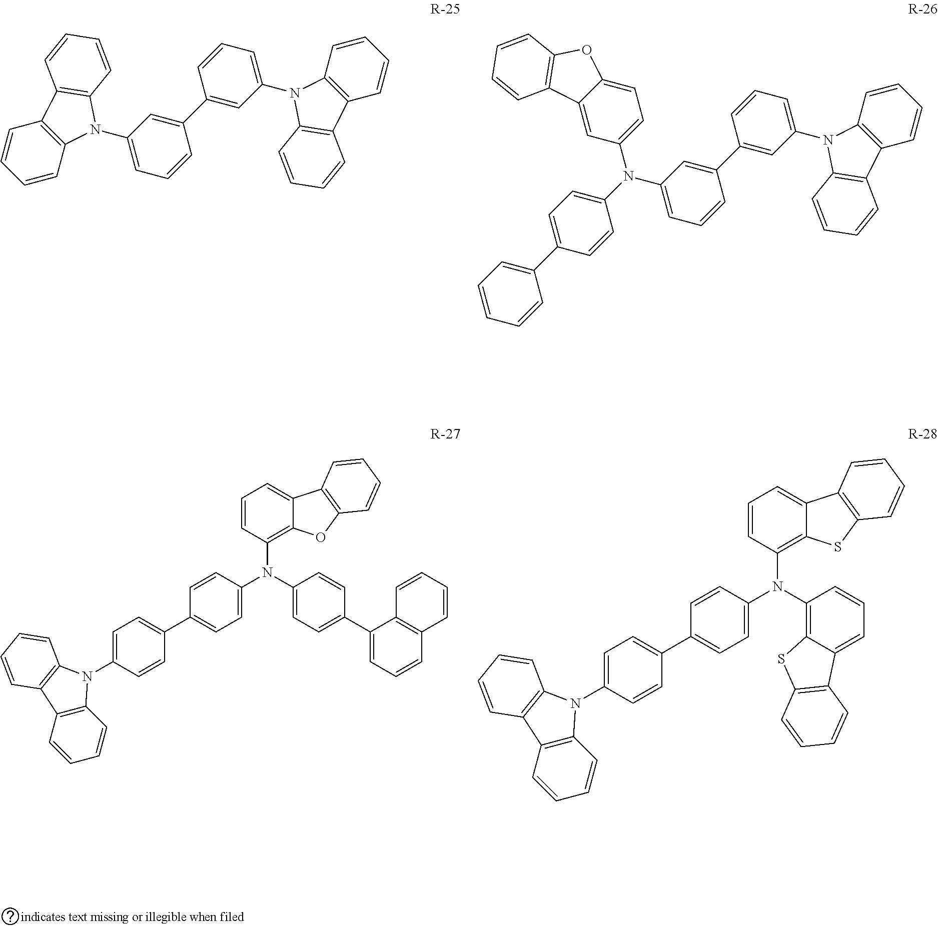 [SMILES: c1cc(-c2cccc(-n3c4ccccc4c4ccccc43)c2)cc(-n2c3ccccc3c3ccccc32)c1 ; c1ccc(-c2ccc(N(c3cccc(-c4cccc(-n5c6ccccc6c6ccccc65)c4)c3)c3ccc4oc5ccccc5c4c3)cc2)cc1 ; c1ccc2c(-c3ccc(N(c4ccc(-c5ccc(-n6c7ccccc7c7ccccc76)cc5)cc4)c4cccc5c4oc4ccccc45)cc3)cccc2c1 ; c1ccc2c(c1)sc1c(N(c3ccc(-c4ccc(-n5c6ccccc6c6ccccc65)cc4)cc3)c3cccc4c3sc3ccccc34)cccc12]

On a glass substrate, ITO with a thickness of about 1,500 Å was patterned and washed with ultra-pure water and treated with UV ozone for about 10 minutes. 2-TNATA was deposited to a thickness of about 600 Å to form a hole injection layer. The Example Compound or Comparative Compound was deposited to a thickness of about 300 Å to form a hole transport layer.

An emission layer with a thickness of about 250 Å was formed using ADN doped with 3% TBP. After that, Alq₃ was deposited to a thickness of about 250 Å to form an electron transport region, and LiF was deposited to a thickness of about 10 Å to form an electron injection layer.

Al was provided to a thickness of about 1,000 Å to form a second electrode. On the second electrode, a capping layer including Compound P4 below was formed to a thickness of about 600 Å.

In the Examples, the hole injection layer, the hole transport layer, the emission layer, the electron transport layer, the electron injection layer and the second electrode were formed using a vacuum deposition apparatus.

(Evaluation of Properties of Light Emitting Diode)

In Table 1, the evaluation results of the light emitting diodes of Example 1 to Example 10, and Comparative Example 1 to Comparative Example 28 are shown. In Table 1, the driving voltage, emission efficiency and diode life of the light emitting diodes thus manufactured are compared and shown. In the evaluation results on the properties of the Examples and Comparative Examples shown in Table 1, the emission efficiency shows efficiency values at a current density of about 10 mA/cm², and the diode life (LT50) shows luminance half life at 1.0 mA/cm².

The current density, voltage and emission efficiency of the light emitting diodes of the Examples and Comparative Examples were obtained using 2400 series Source Meter of Keithley Instrument Co., a chroma meter CS-200 of Konica Minolta Co., and PC Program LabVIEW 2.0 for measurement of National Instrument Co., in Japan in a dark room.

TABLE 1

| Diode | | | | |
|---|---|---|---|---|
| manufacturing | Hole transport layer | Voltage | Efficiency | Life |
| example | material | (V) | (cd/A) | LT50 (h) |
| Example 1 | Example Compound A11 | 5.5 | 7.5 | 1950 |
| Example 2 | Example Compound A16 | 5.2 | 7.1 | 1800 |
| Example 3 | Example Compound B19 | 5.2 | 7.2 | 1900 |
| Example 4 | Example Compound D27 | 5.3 | 7.8 | 1900 |
| Example 5 | Example Compound I7 | 5.3 | 7.4 | 1850 |
| Example 6 | Example Compound L30 | 5.1 | 7.7 | 1800 |
| Example 7 | Example Compound Q14 | 5.4 | 7.6 | 1850 |
| Example 8 | Example Compound Q30 | 5.1 | 7.8 | 1850 |
| Example 9 | Example Compound T13 | 5.7 | 7.1 | 1900 |
| Example 10 | Example Compound X2 | 5.7 | 7.2 | 2000 |
| Comparative | Comparative Compound | 6.0 | 6.3 | 1650 |
| Example 1 | R-1 | | | |
| Comparative | Comparative Compound | 5.9 | 6.9 | 1450 |
| Example 2 | R-2 | | | |
| Comparative | Comparative Compound | 6.0 | 6.6 | 1400 |
| Example 3 | R-3 | | | |
| Comparative | Comparative Compound | 6.1 | 6.8 | 1450 |
| Example 4 | R-4 | | | |
| Comparative | Comparative Compound | 6.5 | 6.0 | 1550 |
| Example 5 | R-5 | | | |
| Comparative | Comparative Compound | 6.1 | 5.1 | 1650 |
| Example 6 | R-6 | | | |
| Comparative | Comparative Compound | 6.4 | 6.3 | 1550 |
| Example 7 | R-7 | | | |
| Comparative | Comparative Compound | 6.3 | 6.8 | 1400 |
| Example 8 | R-8 | | | |
| Comparative | Comparative Compound | 6.3 | 6.5 | 1500 |
| Example 9 | R-9 | | | |
| Comparative | Comparative Compound | 6.4 | 5.8 | 1450 |
| Example 10 | R-10 | | | |
| Comparative | Comparative Compound | 6.5 | 6.5 | 1400 |
| Example 11 | R-11 | | | |
| Comparative | Comparative Compound | 5.9 | 6.9 | 1650 |
| Example 12 | R-12 | | | |
| Comparative | Comparative Compound | 6.1 | 5.9 | 1500 |
| Example 13 | R-13 | | | |
| Comparative | Comparative Compound | 6.4 | 5.6 | 1400 |
| Example 14 | R-14 | | | |
| Comparative | Comparative Compound | 6.3 | 5.9 | 1550 |
| Example 15 | R-15 | | | |
| Comparative | Comparative Compound | 6.6 | 6.4 | 1550 |
| Example 16 | R-16 | | | |
| Comparative | Comparative Compound | 6.5 | 6.8 | 1550 |
| Example 17 | R-17 | | | |
| Comparative | Comparative Compound | 6.2 | 6.6 | 1500 |
| Example 18 | R-18 | | | |
| Comparative | Comparative Compound | 6.0 | 6.9 | 1400 |
| Example 19 | R-19 | | | |
| Comparative | Comparative Compound | 6.1 | 5.9 | 1750 |
| Example 20 | R-20 | | | |
| Comparative | Comparative Compound | 6.0 | 6.4 | 1650 |
| Example 21 | R-21 | | | |
| Comparative | Comparative Compound | 5.9 | 6.0 | 1350 |
| Example 22 | R-22 | | | |
| Comparative | Comparative Compound | 6.0 | 6.2 | 1650 |
| Example 23 | R-23 | | | |
| Comparative | Comparative Compound | 5.8 | 6.3 | 1600 |
| Example 24 | R-24 | | | |
| Comparative | Comparative Compound | 6.0 | 6.0 | 1400 |
| Example 25 | R-25 | | | |
| Comparative | Comparative Compound | 6.1 | 6.8 | 1500 |
| Example 26 | R-26 | | | |
| Comparative | Comparative Compound | 5.8 | 6.8 | 1450 |
| Example 27 | R-27 | | | |
| Comparative | Comparative Compound | 5.9 | 6.9 | 1400 |
| Example 28 | R-28 | | | |

Referring to the results of Table 1, it could be found that the light emitting diodes of the Examples using the amine compounds of embodiments of the disclosure as materials for a hole transport layer showed a low driving voltage, excellent diode efficiency and improved diode-life characteristics. For example, referring to Table 1, it could be confirmed that the light emitting diodes of Example 1 to Example 10 showed a lower driving voltage, longer life and higher efficiency when compared with the light emitting diodes of Comparative Example 1 to Comparative Example 28.

The amine compound according to an embodiment is a diamine compound having a biphenyl skeleton and a dibenzoheterole group and may accomplish long life and high efficiency properties as well as low driving voltage properties at the same time. The amine compound of an embodiment includes a dibenzoheterole group having excellent hole transport capacity, and may show improved hole transport capacity while maintaining low voltage properties of the diamine. The amine compound of an embodiment includes a dibenzoheterole group, and the migration of holes from a hole transport region to an emission layer may be accelerated, and the emission efficiency of a light emitting diode may be improved. Since biphenyl was included as a linking moiety connecting the nitrogen atoms of two amine groups, the stability of a compound was improved, and the life of a light emitting diode was increased.

As shown in Example 1 to Example 10, the Example Compounds include a dibenzoheterole group in a molecule, and the increase of emission efficiency could be confirmed. Such increase of the emission efficiency is thought to be achieved because a heteroatom included in the dibenzoheterole group enhances the hole transport capacity of a whole molecule, and the recombination probability of holes and electrons in an emission layer is increased. For example, the light emitting diodes of Examples 4, 6 and 8 included the Example Compounds further including a carbazole group in a molecule, and even higher emission efficiency properties were shown. Such improved emission efficiency properties are thought to be achieved because intermolecular stacking is increased due to the planarity of the carbazole group, hole mobility is increased due to the decrease of intermolecular distance, and hole transport capacity is enhanced.

When compared with the Examples, Comparative Example 1 showed degraded results of both diode emission efficiency and diode life. These results were thought to be obtained because a moiety having high nucleophilicity due to two oxygen atoms of benzobisbenzofuran reacted with the radical cations of adjacent molecules produced during driving a diode, and diode deterioration was generated.

When compared with the Examples, Comparative Examples 2, 19 and 26 showed results of markedly reduced diode life. In case where a nitrogen atom and the heteroatom of a dibenzoheterole group are combined at para positions as in Comparative Compounds R-2, R-19, and R-26, it is thought that an aromatic ring easily becomes electron excess due to the heteroatom, and reactivity is high, reaction is carried out during driving a diode, and diode deterioration is generated. In contrast, in case of the Example Compounds, 4-dibenzoheterol has a twist between the conjugation of a nitrogen atom and a heteroatom, and this twist prevents the electron excess due to the heteroatom combined at an ortho position, and high hole mobility and tolerance to deterioration may be shown at the same time.

When compared with the Examples, the diode life was reduced in Comparative Examples 3, 4 and 22. It is though that in case where two or more nitrogen are combined in one aromatic ring as in Comparative Compounds R-3, R-4, and R-22, a moiety having high nucleophilicity was present, reaction was processed when the radical cations of an adjacent molecule were produced during driving a diode, and diode deterioration was generated.

In Comparative Example 5, both the emission efficiency and diode life of the diode were degraded. Benzonaphthofuran or benzonaphthothiophene is a substituent having high deposition temperature due to planarity. For example, it is thought that Comparative Compound R-5 included a substituent having relatively high deposition temperature such as benzonaphthofuran, benzonaphthothiophene, and carbazole, and decomposition was generated during the deposition process during the manufacture of a diode, and Comparative Example 5 showed degraded diode properties.

In Comparative Example 6, the emission efficiency of a diode was largely degraded. Such degradation of emission efficiency is thought to be shown because Comparative Compound R-6 included pyrene as a substituent, and energy from an emission layer was absorbed.

In Comparative Examples 7 and 11, both the emission efficiency and life of the diodes were degraded. It is thought that a substituent other than an amine group was substituted at biphenyl in Comparative Compounds R-7 and R-11, and deposition temperature was increased, and the decomposition of a compound was generated during a deposition process. For example, when comparing the Example Compounds of the disclosure with Comparative Compounds in Comparative Examples 3, 4, 7, 11 and 22, it could be found that both high material stability and low deposition temperature may be achieved if a biphenyl group is included as a linking moiety between nitrogen atoms of two amine groups as in the Example Compounds.

In Comparative Examples 8 and 16, both the emission efficiency and life characteristics were degraded. The Comparative Compounds used in Comparative Examples 8 and 16 additionally included a dibenzoheterole group disposed at the outside with a phenyl linker therebetween and required high deposition temperature. Accordingly, an additional dibenzoheterole group was included and it is thought that the deposition temperature was increased due to the increase of planarity when compared with a case of including and an aryl group having no crosslinking bonds by a heteroatom, and the decomposition of a compound during a deposition process was generated according to the structural characteristics of such Comparative Compounds.

In Comparative Compound 9, both the emission efficiency and life characteristics of the light emitting diode were degraded. Comparative Compound R-9 has a skeleton in which two nitrogen atoms are combined with a dibenzoheterole group, and accordingly, has highly nucleophilic moiety. It is thought that due to the structural characteristics of such a compound, reaction was carried out when radical cations of an adjacent molecule are produced during driving a diode, and diode deterioration was generated, and accordingly, diode properties were degraded.

In Comparative Examples 10, 13 and 14, both the emission efficiency and diode life of the light emitting diodes were degraded. It is thought that C—C bond combined with a quaternary carbon corresponding to a benzyl position in a fluorine skeleton was thermally unstable and decomposed during deposition, and the diode performance of the Comparative Examples was degraded.

Comparative Compound R-12 used in Comparative Example 12 corresponds to an amine compound including both a biphenyl group and a dibenzoheterole group similar to embodiments of the disclosure, but diode life was degraded when compared with the Examples. In case of an aromatic ring having a heteroatom, electron introduction is easy, and weak electron tolerance is shown when compared with an aryl group, and different from a sulfur atom, for example, an oxygen atom among heteroatoms does not have an empty orbital with respect to electrons, and tolerance to electrons leaking from host is low. Accordingly, in order to restrain the generation of the decomposition of the compound, in case of a dibenzoheterole group including an oxygen atom as a heteroatom is directly bonded to the nitrogen atom of amine, the stabilization of a molecule by distributing electrons to another substituent is required. Stabilization through a wide distribution of electrons may be limited only with a phenyl group, and in the case of Comparative Compound R-12, it is thought that a phenyl group was included as an additional substituent to the dibenzoheterole group including an oxygen atom as a heteroatom, and degradation of the diode was generated during the driving of a diode.

In Comparative Example 15, both the emission efficiency and life of a light emitting diode were degraded. A 7H-dibenzo[c,g]carbazole skeleton is a skeleton having high planarity, and it is thought that in case of Comparative Compound R-15 including the same, the deposition temperature was significantly elevated, the compound was decomposed during a deposition process, and diode performance was degraded. In Comparative Example 17, both the emission efficiency and life of a light emitting diode were degraded. A 4H-benzo[def]carbazole skeleton is also a skeleton having high planarity, and it is thought that the deposition temperature was significantly elevated, the compound was decomposed during deposition, and diode performance was degraded.

In Comparative Example 18, both the emission efficiency and life of the light emitting diode were degraded. A dibenzoheterole group disposed outside with a phenyl linker therebetween largely elevates the deposition temperature. The carbazole group also has high planarity when compared with an amine group and elevates the deposition temperature, and it is thought that the decomposition according to the increase of the deposition temperature was generated due to the combination of the dibenzoheterole group and the carbazole group via the phenyl linker. The diode properties were degraded in Comparative Example 16 by similar reasons to those in Comparative Example 18.

In Comparative Examples 20 and 21, the emission efficiency of the diodes was degraded. Different from the Example Compounds of the disclosure, in case of Comparative Compounds R-20 and R-21, a dibenzoheterole group having excellent hole transport capacity was not included, and diode efficiency was not improved.

The Comparative Compounds used in Comparative Examples 23, 24, 27, and 28 correspond to amine compounds including both a biphenyl group and a dibenzoheterole group similar to the Example Compounds, but degraded properties of diode life were shown when compared with the Examples. In case where any of one nitrogen atom of an amine group is combined at a para position with respect to a biphenyl group as the Comparative Compounds used in Comparative Examples 23, 24, 27, and 28, the HOMO orbital of the nitrogen atom may be expanded to the other phenyl group combined with another one nitrogen atom as well as a phenyl group directly connected. Accordingly, the phenyl group is influenced by two nitrogen atoms and has a highly nucleophilic moiety, and diode deterioration may be generated during driving the diode. Accordingly, Comparative Examples 23, 24, 27, and 28 are thought to show degraded diode properties when compared with the Examples.

In contrast, in the Example Compounds in Examples 5, 6, 7, 8, and 9, it is thought that nitrogen was combined at an ortho position with respect to a biphenyl group, and a structure in which a highly nucleophilic moiety was not generated due to twist.

Comparative Compound R-25 in Comparative Example 25 has a structure in which carbazole is combined with a biphenyl group similar to the Example Compounds, but Comparative Example 25 showed degraded results of both emission efficiency and life when compared with the Examples. As described above, carbazole is a substituent of which deposition temperature may be easily elevated due to planarity when compared with an amine group, and the compound includes multiple carbazole groups having such properties and has high deposition temperature properties, and accordingly, it is thought that the decomposition of a material was generated, and diode properties were degraded.

As described above, Example 1 to Example 10 showed improved emission efficiency and emission life when compared with Comparative Example 1 to Comparative Example 28. For example, the Example Compounds having the structure of a diamine compound including both a biphenyl skeleton and a dibenzoheterole group may be used as a material of a light emitting diode and may improve both diode efficiency and diode life.

The amine compound according to an embodiment includes a biphenyl group as a linking moiety and amine groups bonded to each of the benzene rings of the biphenyl group, and has a molecular structure in which at least one of the amine groups includes a dibenzoheterole group, thereby contributing to the decrease of a voltage and the increase of life and efficiency properties of a light emitting diode. The light emitting diode according to an embodiment includes the amine compound of an embodiment and may show long life and high efficiency properties at the same time.

The light emitting diode of an embodiment includes the amine compound of an embodiment in a hole transport region and may show high efficiency and long-life characteristics.

The amine compound of an embodiment may improve emission efficiency and service life of a light emitting diode.

Embodiments have been disclosed herein, and although terms are employed, they are used and are to be interpreted in a generic and descriptive sense only and not for purpose of limitation. In some instances, as would be apparent by one of ordinary skill in the art, features, characteristics, and/or elements described in connection with an embodiment may be used singly or in combination with features, characteristics, and/or elements described in connection with other embodiments unless otherwise specifically indicated. Accordingly, it will be understood by those of ordinary skill in the art that various changes in form and details may be made without departing from the spirit and scope of the disclosure as set forth in the following claims.

What is claimed is:

1. A light emitting diode, comprising:
a first electrode;
a second electrode disposed on the first electrode; and
at least one functional layer disposed between the first electrode and the second electrode, the at least one functional layer comprising an amine compound represented by Formula 1: 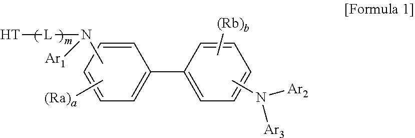
wherein in Formula 1,
a and b are each independently an integer from 0 to 4,
Ra and Rb are each independently a hydrogen atom or a deuterium atom,
m is 0 or 1,
L is a substituted or unsubstituted arylene group of 6 to 40 ring-forming carbon atoms, and
HT is a group represented by Formula 2: 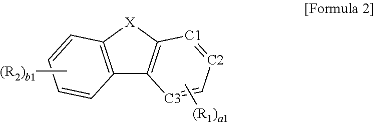
wherein in Formula 2,
C1 to C3 each represent a carbon atom in an aromatic ring,
a position selected from C1 to C3 is bonded to Formula 1,
X is O or S,
a1 is an integer from 0 to 3,
b1 is an integer from 0 to 4, and
R₁ and R₂ are each independently a hydrogen atom, a deuterium atom, a substituted or unsubstituted alkyl group of 1 to 20 carbon atoms, a substituted or unsubstituted alkenyl group of 2 to 20 carbon atoms, or a substituted or unsubstituted aryl group of 6 to 40 ring-forming carbon atoms, or multiple R₁ groups or multiple R₂ groups are combined with each other to form an aromatic ring,
wherein in Formula 1,
in case where m is 1, Ar₁ to Ar₃ are each independently a substituted or unsubstituted aryl group of 6 to 40 ring-forming carbon atoms,
in case where m is 0,
Ar₁ to Ar₃ are each independently a substituted or unsubstituted aryl group of 6 to 40 ring-forming carbon atoms, or a substituted or unsubstituted heteroaryl group of 5 to 40 ring-forming carbon atoms, or
Ar₁ is a substituted or unsubstituted aryl group of 6 to 40 ring-forming carbon atoms, or a substituted or unsubstituted heteroaryl group of 5 to 40 ring-forming carbon atoms, and Ar₂ and Ar₃ are combined with an adjacent group to form a carbazole derivative group except for groups represented by CZ-1 to CZ-3,
in case where m is 0, none of Ar₁ to Ar₃ are a substituted or unsubstituted fluorenyl group, a substituted or unsubstituted pyrenyl group, an unsubstituted carbazolyl group, a 2-dibenzofuranyl group, and a 2-dibenzothiophenyl group, and
in case where m is 0, X is not O, and Ar₁ is not an unsubstituted phenyl group, and wherein in CZ-1 to CZ-3, [symbol]
represents a binding site to a neighboring atom:

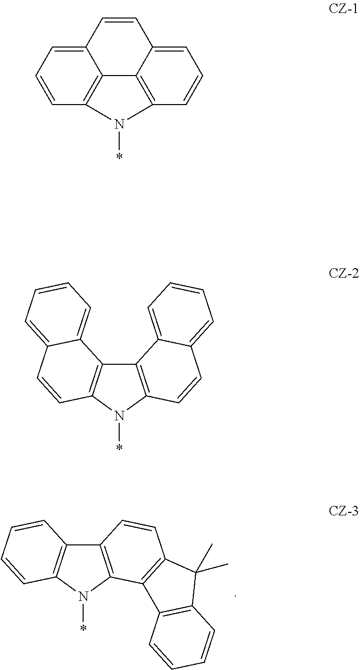 [SMILES: *n1c2cccc3ccc4cccc1c4c32 ; *n1c2ccc3ccccc3c2c2c3ccccc3ccc21 ; *n1c2ccccc2c2ccc3c(c21)-c1ccccc1C3(C)C ; "."]

2. The light emitting diode of claim 1, wherein
the at least one functional layer comprises:
an emission layer;
a hole transport region disposed between the first electrode and the emission layer; and
an electron transport region disposed between the emission layer and the second electrode, and
the hole transport region comprises the amine compound.

3. The light emitting diode of claim 2, wherein
the hole transport region comprises at least one of a hole injection layer, a hole transport layer, and an electron blocking layer, and
at least one of the hole injection layer, the hole transport layer, and the electron blocking layer comprises the amine compound.

4. The light emitting diode of claim 1, wherein Formula 1 is represented by one of Formula 1-1 to Formula 1-4: 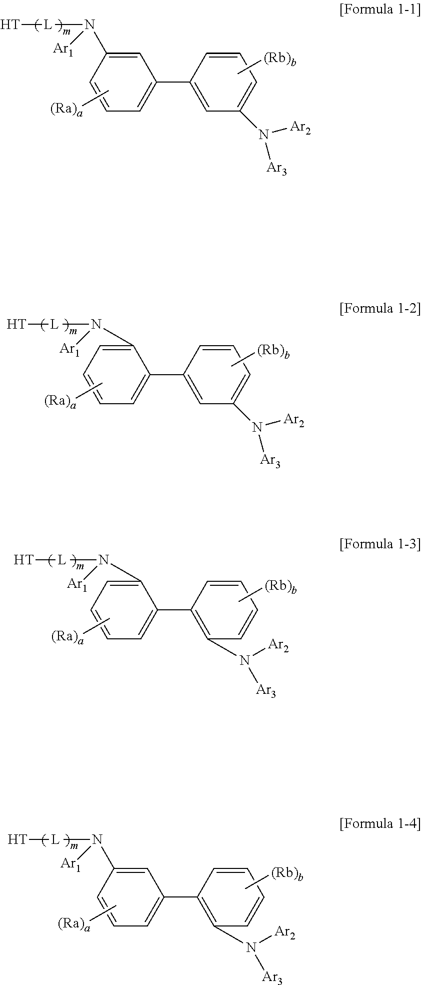
wherein in Formula 1-1 to Formula 1-4,
a, b, Ra, Rb, m, L, Ar₁, Ar₂, Ar₃, and HT are the same as defined in connection with Formula 1 and Formula 2.

5. The light emitting diode of claim 1, wherein Formula 1 is represented by Formula 1-A or Formula 1-B: 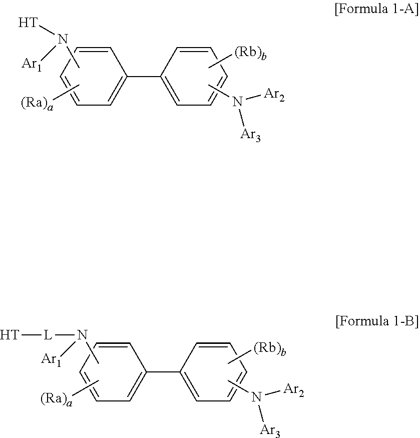
wherein in Formula 1-A and Formula 1-B,
a, b, Ra, Rb, L, Ar₁, Ar₂, Ar₃, and HT are the same as defined in connection with Formula 1 and Formula 2.

6. The light emitting diode of claim 5, wherein in Formula 1-B,
L is represented by one of L-1 to L-3: 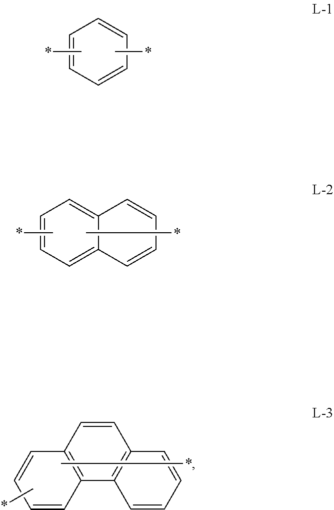
wherein in L-1 to L-3, [symbol]
represents a binding site to a neighboring atom.

7. The light emitting diode of claim 1, wherein Formula 2 is represented by one of Formula 2-1 to Formula 2-3: 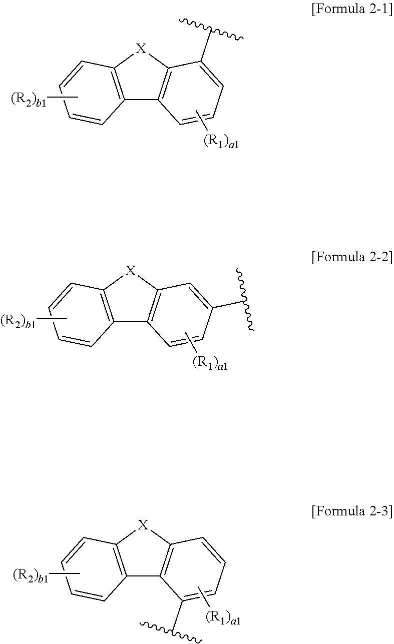
wherein in Formula 2-1 to Formula 2-3,
X, a1, b1, R₁, and R₂ are the same as defined in connection with Formula 2, and 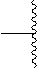
represents a binding site to a neighboring atom.

8. The light emitting diode of claim 1, wherein Formula 2 is represented by one of Formula 2-a to Formula 2-e: 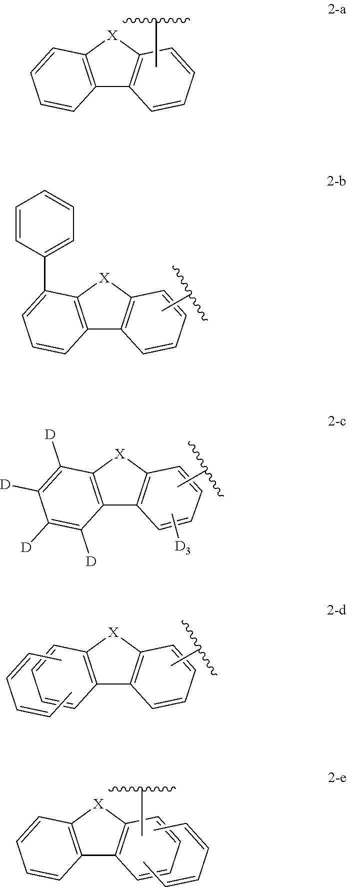
wherein in Formula 2-a to Formula 2-e,
X is the same as defined in connection with Formula 2, and 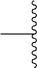
represents a binding site to Formula 1 at a position selected from C1 to C3 in Formula 2.

9. The light emitting diode of claim 1, wherein
at least one of Ra, Rb, R₁, and R₂ is a deuterium atom, or
at least one of Ar₁ to Ar₃ is an aryl group of 6 to 40 ring-forming carbon atoms substituted with at least one deuterium atom.

10. The light emitting diode of claim 1, wherein
a and b are each 4, and
Ra and Rb are each deuterium atoms.

11. The light emitting diode of claim 1, wherein,
in case where m is 0, and at least one of Ar₁ to Ar₃ is a substituted or unsubstituted heteroaryl group of 5 to 40 ring-forming carbon atoms,
the heteroaryl group comprises O or S as a heteroatom.

12. The light emitting diode of claim 2, wherein the emission layer comprises a compound represented by Formula E-1: 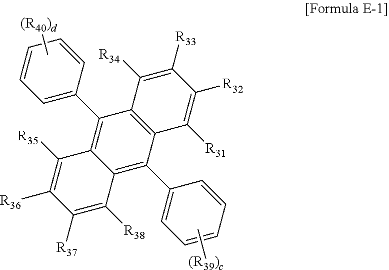
wherein in Formula E-1,
R₃₁ to R₄₀ are each independently a hydrogen atom, a deuterium atom, a halogen atom, a substituted or unsubstituted silyl group, a substituted or unsubstituted alkyl group of 1 to 10 carbon atoms, a substituted or unsubstituted aryl group of 6 to 30 ring-forming carbon atoms, or a substituted or unsubstituted heteroaryl group of 2 to 30 ring-forming carbon atoms, or are combined with an adjacent group to form a ring, and
c and d are each independently an integer from 0 to 5.

13. The light emitting diode of claim 1, wherein the amine compound is one selected from Compound Group 1A to Compound Group 1Y:

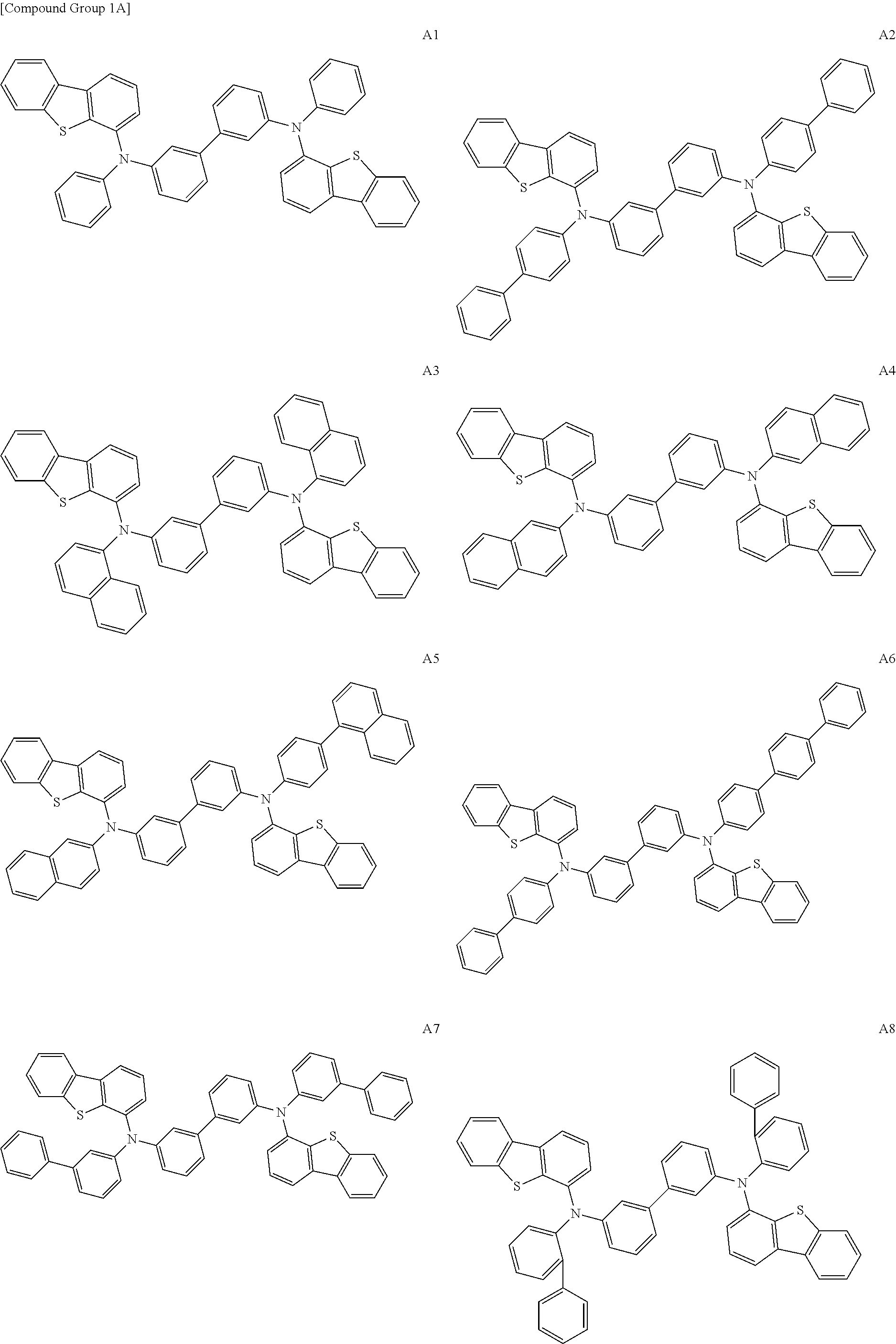

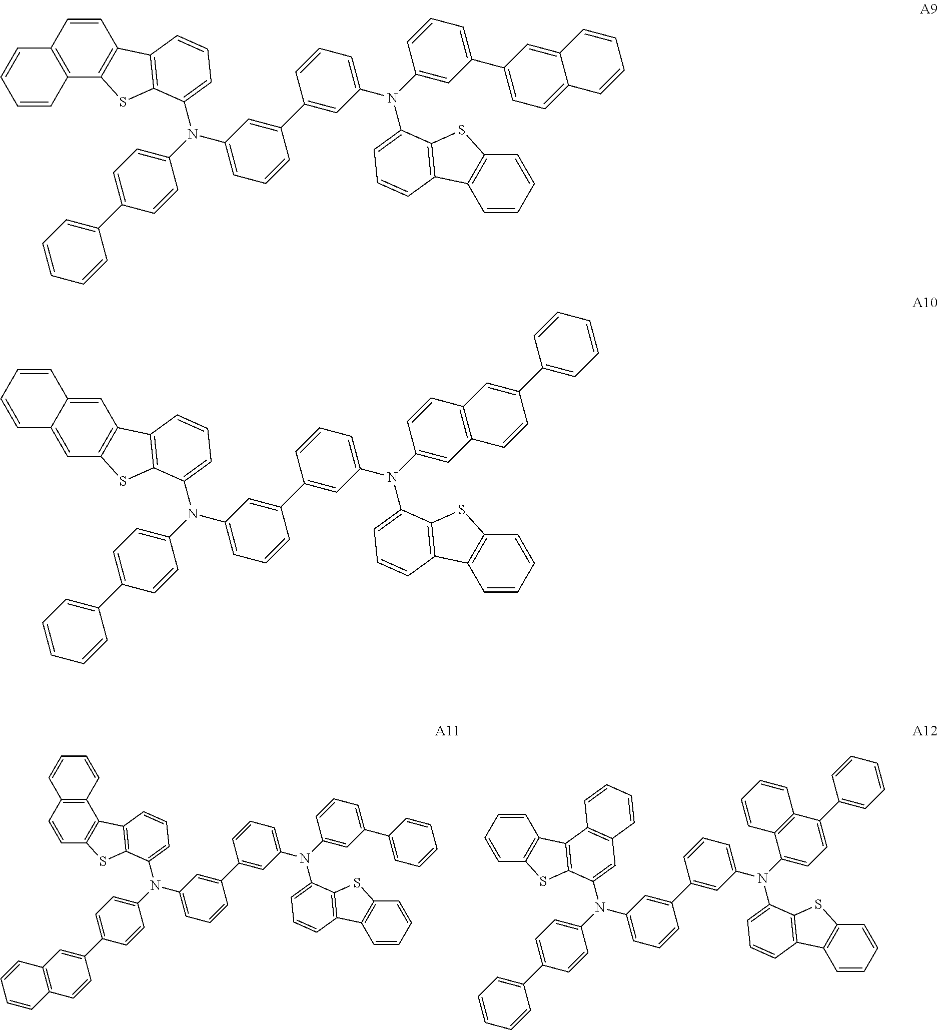 [SMILES: c1ccc(-c2ccc(N(c3cccc(-c4cccc(N(c5cccc(-c6ccc7ccccc7c6)c5)c5cccc6c5sc5ccccc56)c4)c3)c3cccc4c3sc3c5ccccc5ccc43)cc2)cc1 ; c1ccc(-c2ccc(N(c3cccc(-c4cccc(N(c5ccc6cc(-c7ccccc7)ccc6c5)c5cccc6c5sc5ccccc56)c4)c3)c3cccc4c3sc3cc5ccccc5cc34)cc2)cc1 ; c1ccc(-c2cccc(N(c3cccc(-c4cccc(N(c5ccc(-c6ccc7ccccc7c6)cc5)c5cccc6c5sc5ccc7ccccc7c56)c4)c3)c3cccc4c3sc3ccccc34)c2)cc1 ; c1ccc(-c2ccc(N(c3cccc(-c4cccc(N(c5ccc(-c6ccccc6)c6ccccc56)c5cccc6c5sc5ccccc56)c4)c3)c3cc4ccccc4c4c3sc3ccccc34)cc2)cc1]

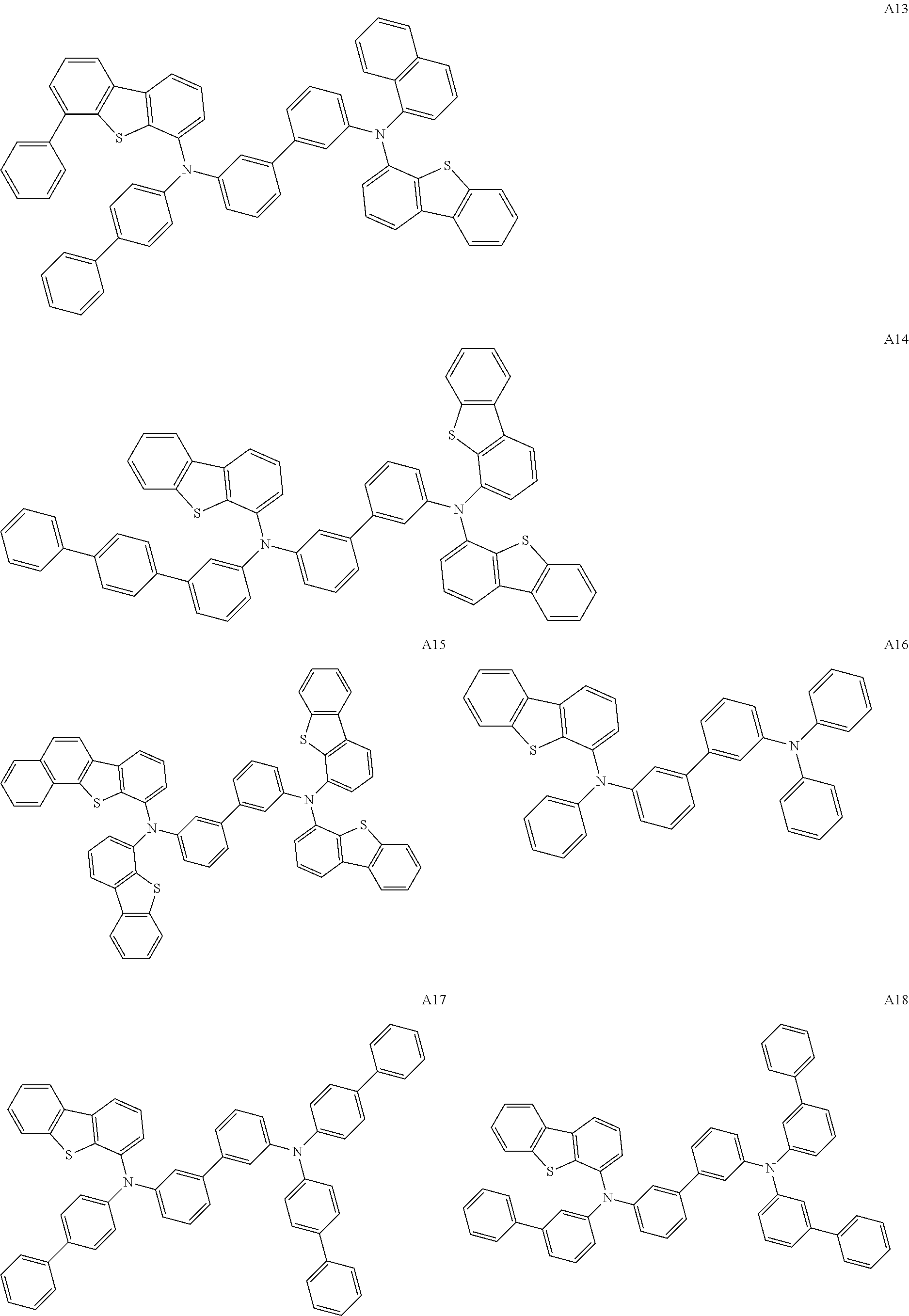

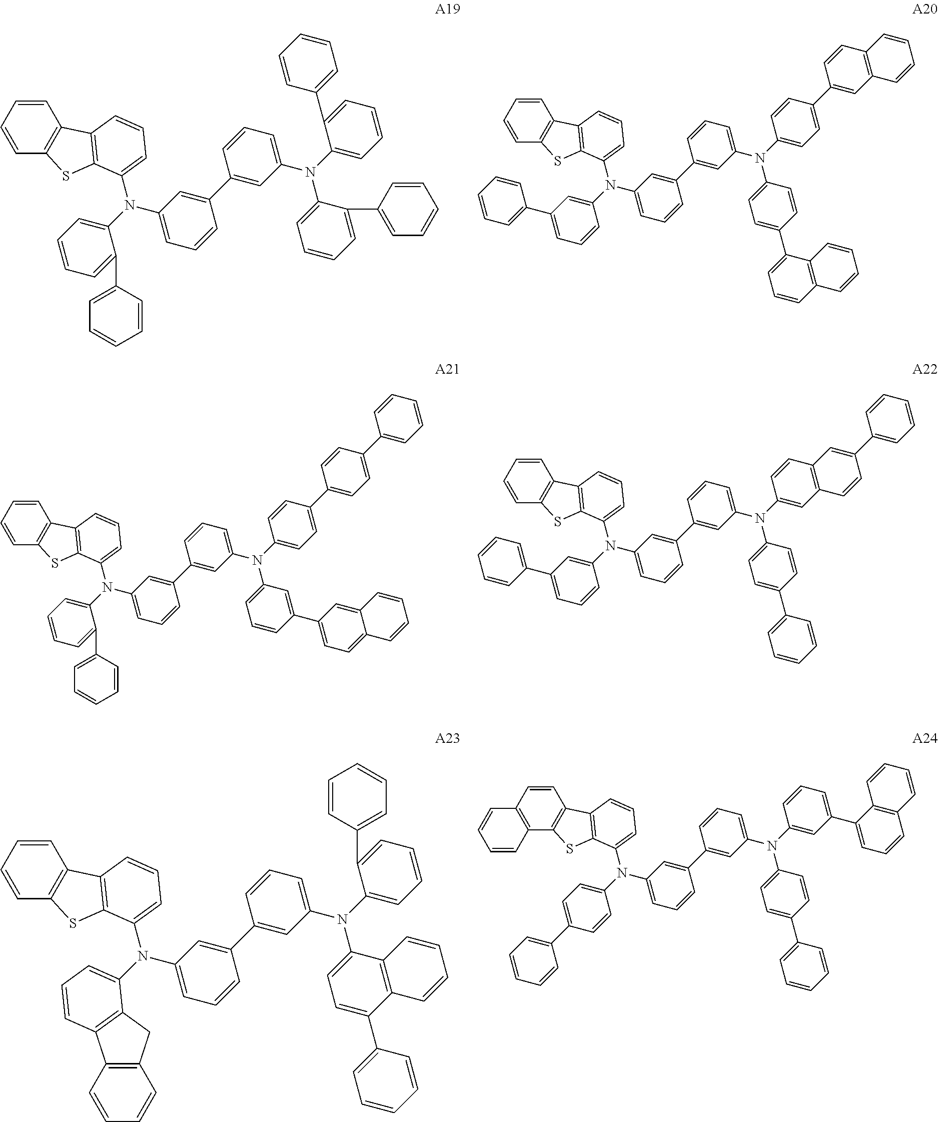

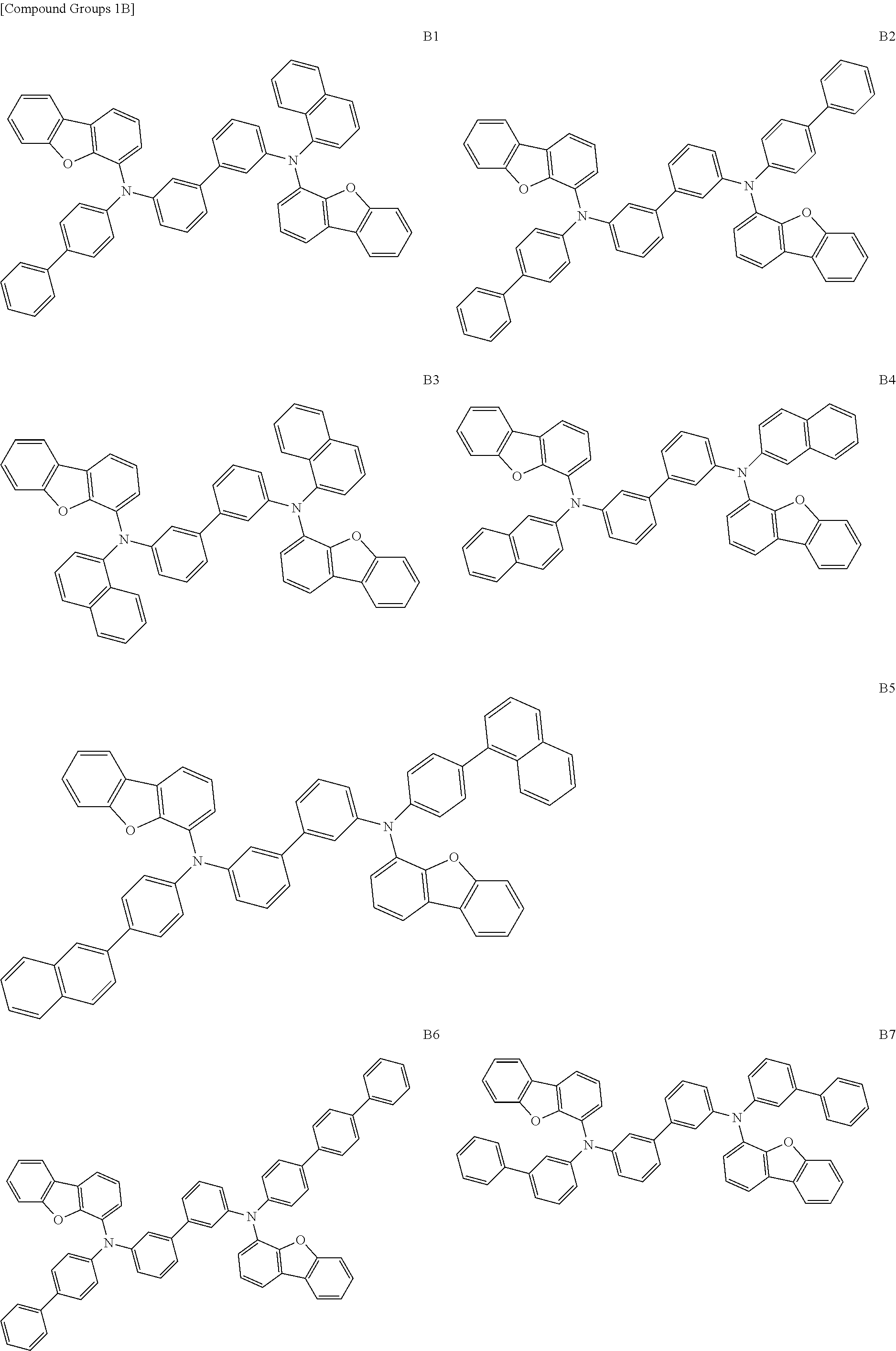

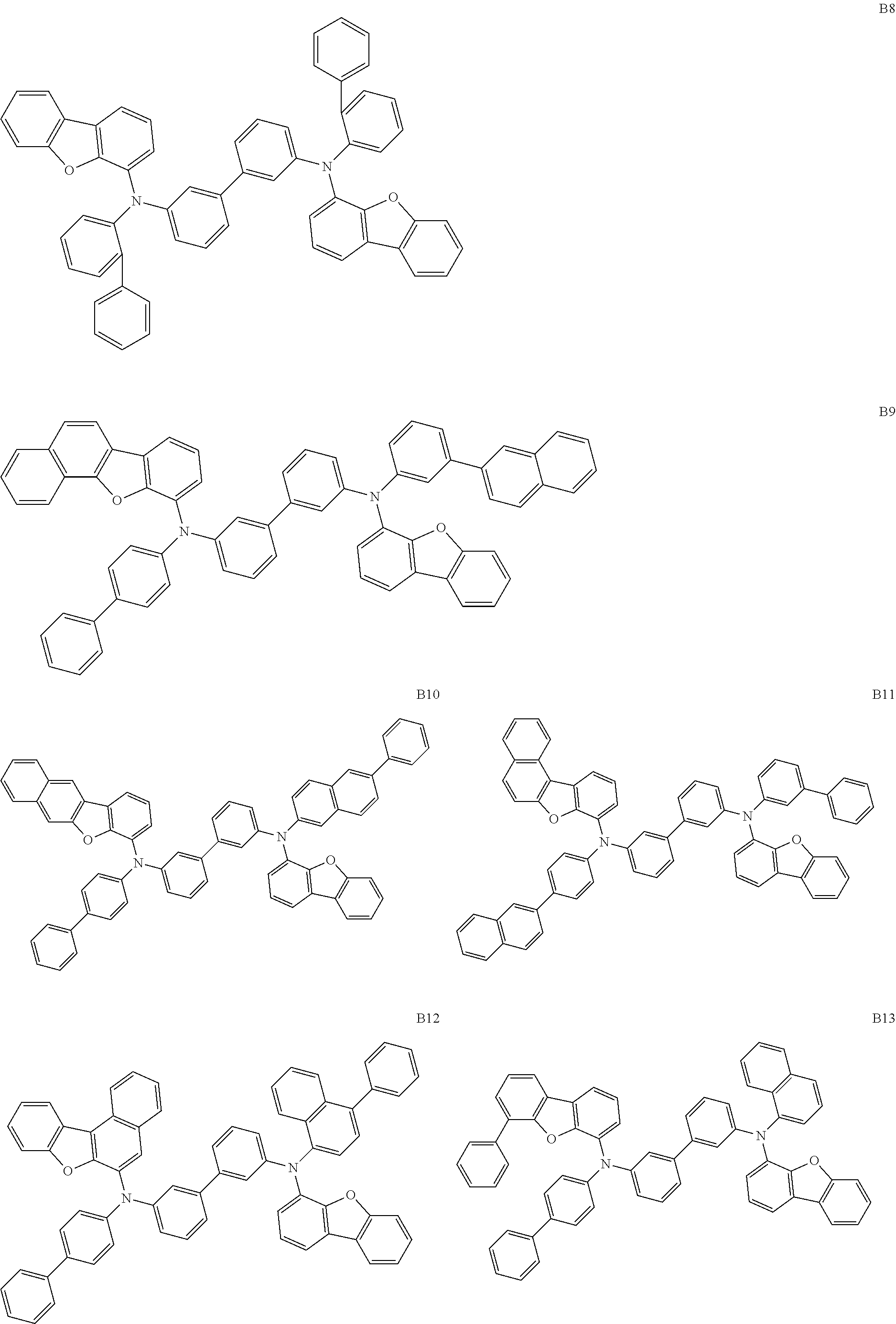

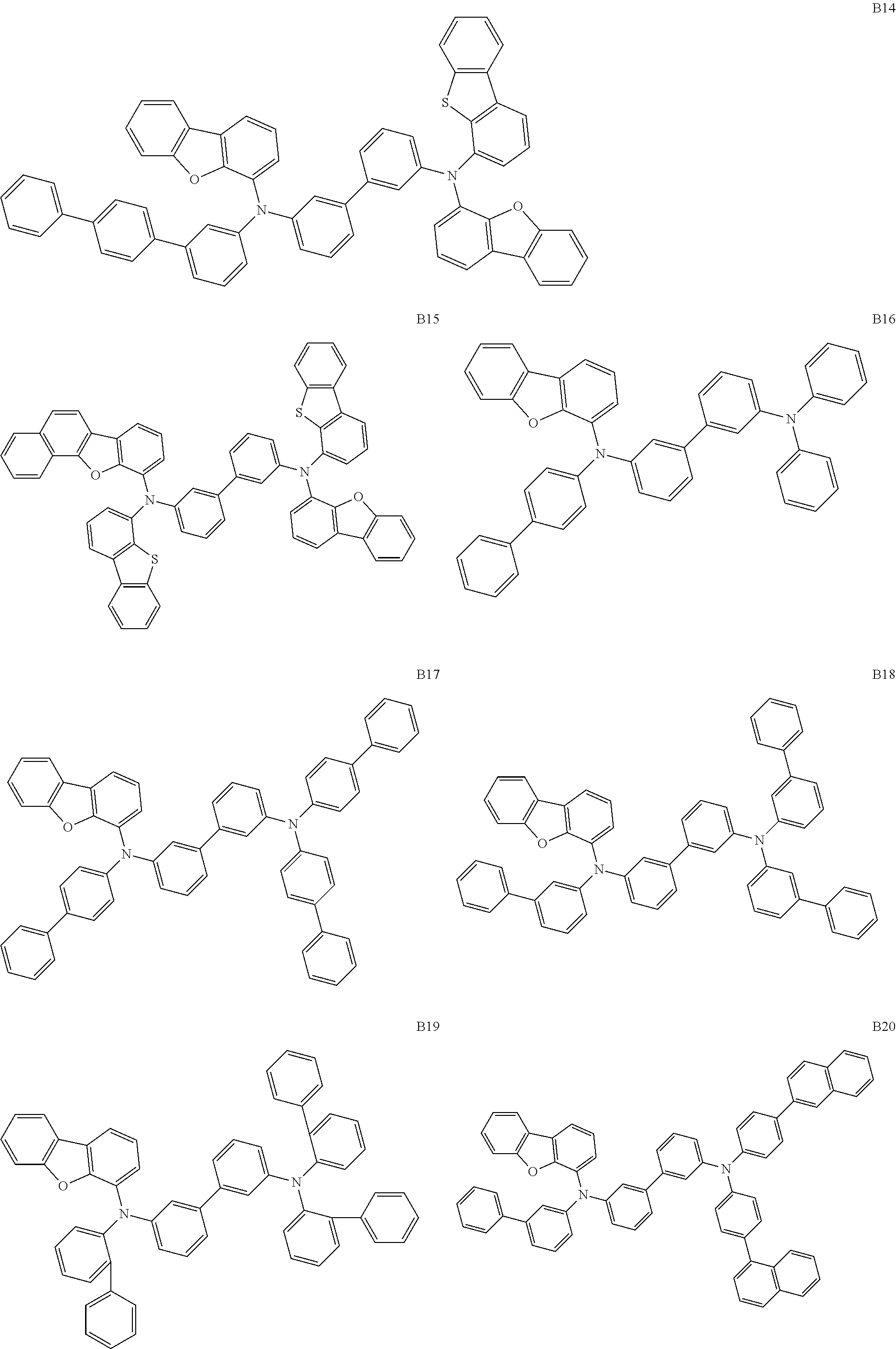

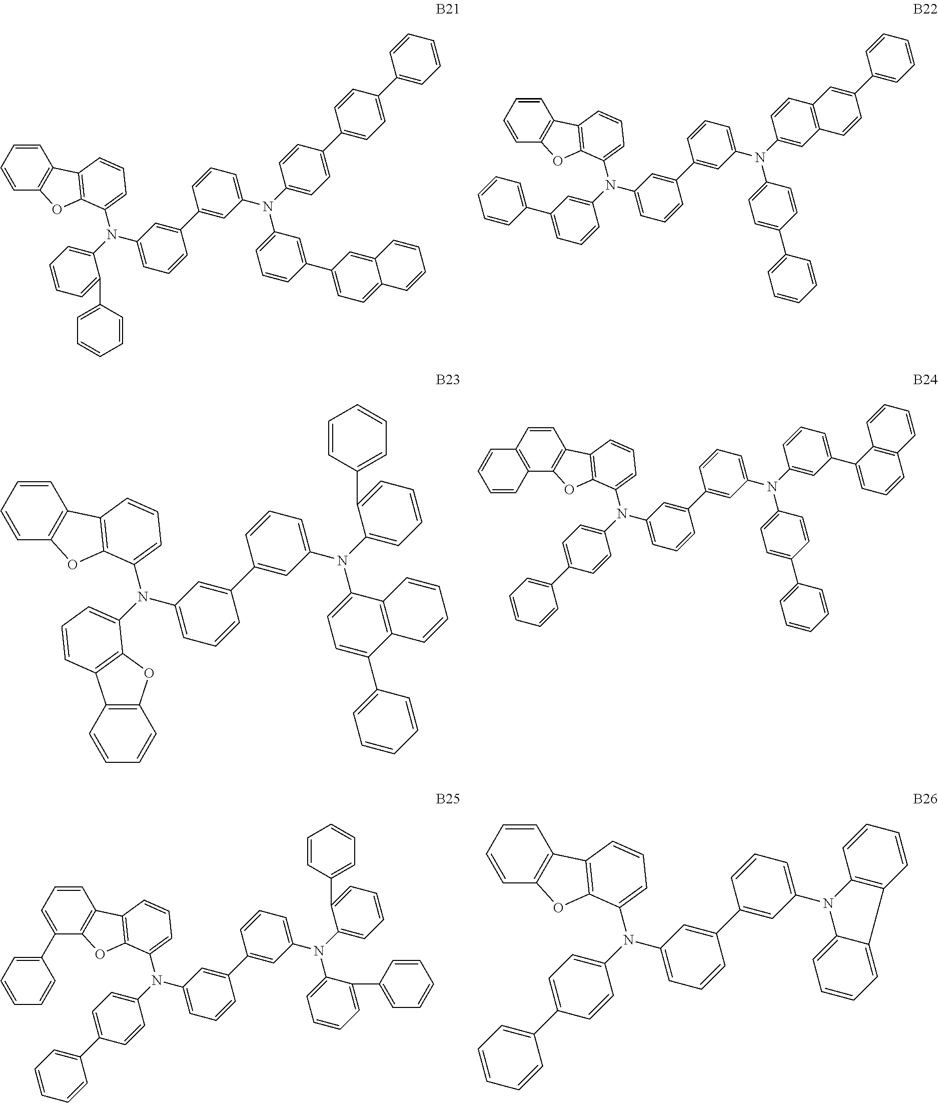

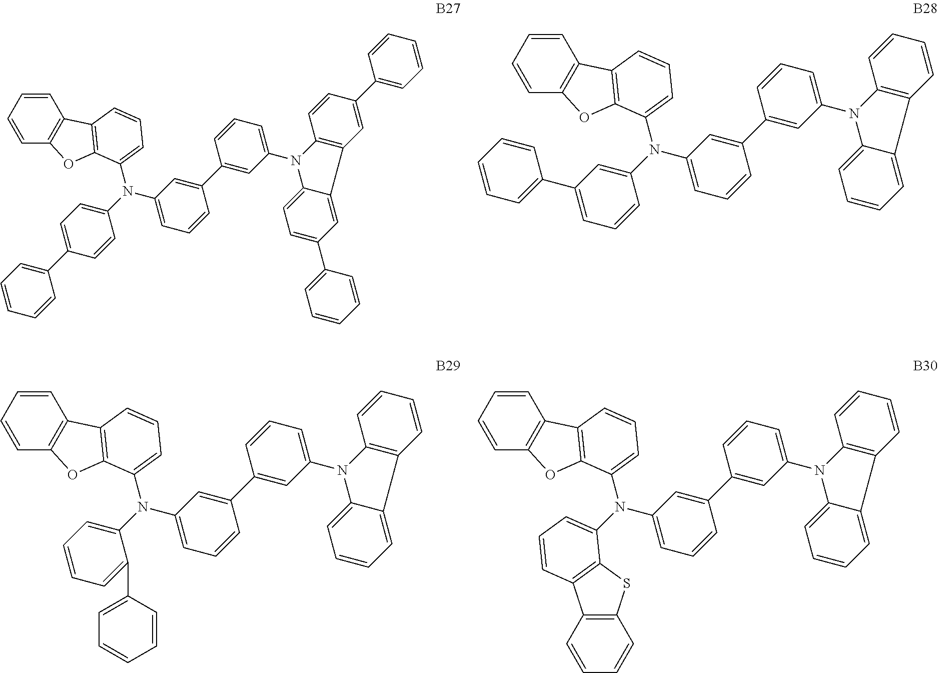 [SMILES: c1ccc(-c2ccc(N(c3cccc(-c4cccc(-n5c6ccc(-c7ccccc7)cc6c6cc(-c7ccccc7)ccc65)c4)c3)c3cccc4c3oc3ccccc34)cc2)cc1 ; c1ccc(-c2cccc(N(c3cccc(-c4cccc(-n5c6ccccc6c6ccccc65)c4)c3)c3cccc4c3oc3ccccc34)c2)cc1 ; c1ccc(-c2ccccc2N(c2cccc(-c3cccc(-n4c5ccccc5c5ccccc54)c3)c2)c2cccc3c2oc2ccccc23)cc1 ; c1cc(-c2cccc(-n3c4ccccc4c4ccccc43)c2)cc(N(c2cccc3c2oc2ccccc23)c2cccc3c2sc2ccccc23)c1]

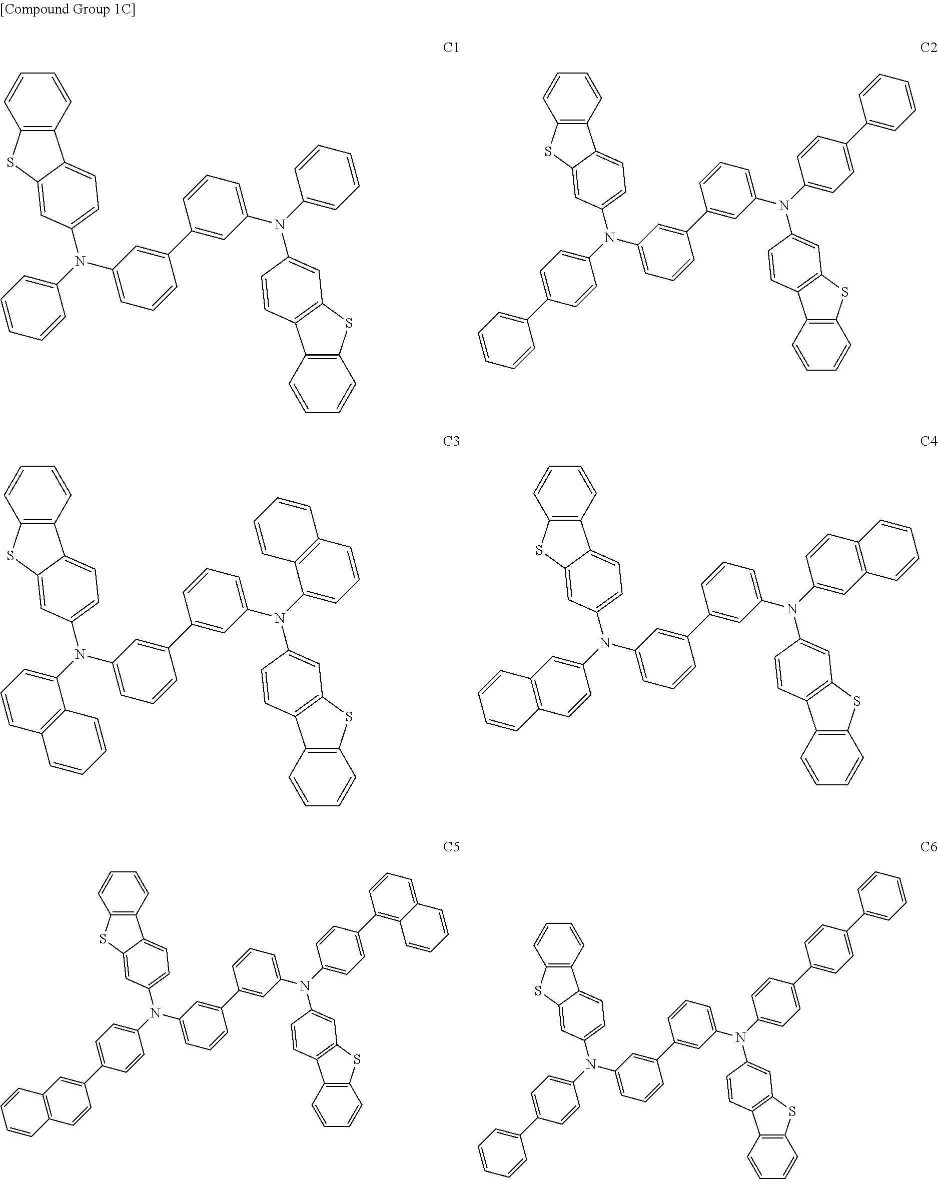

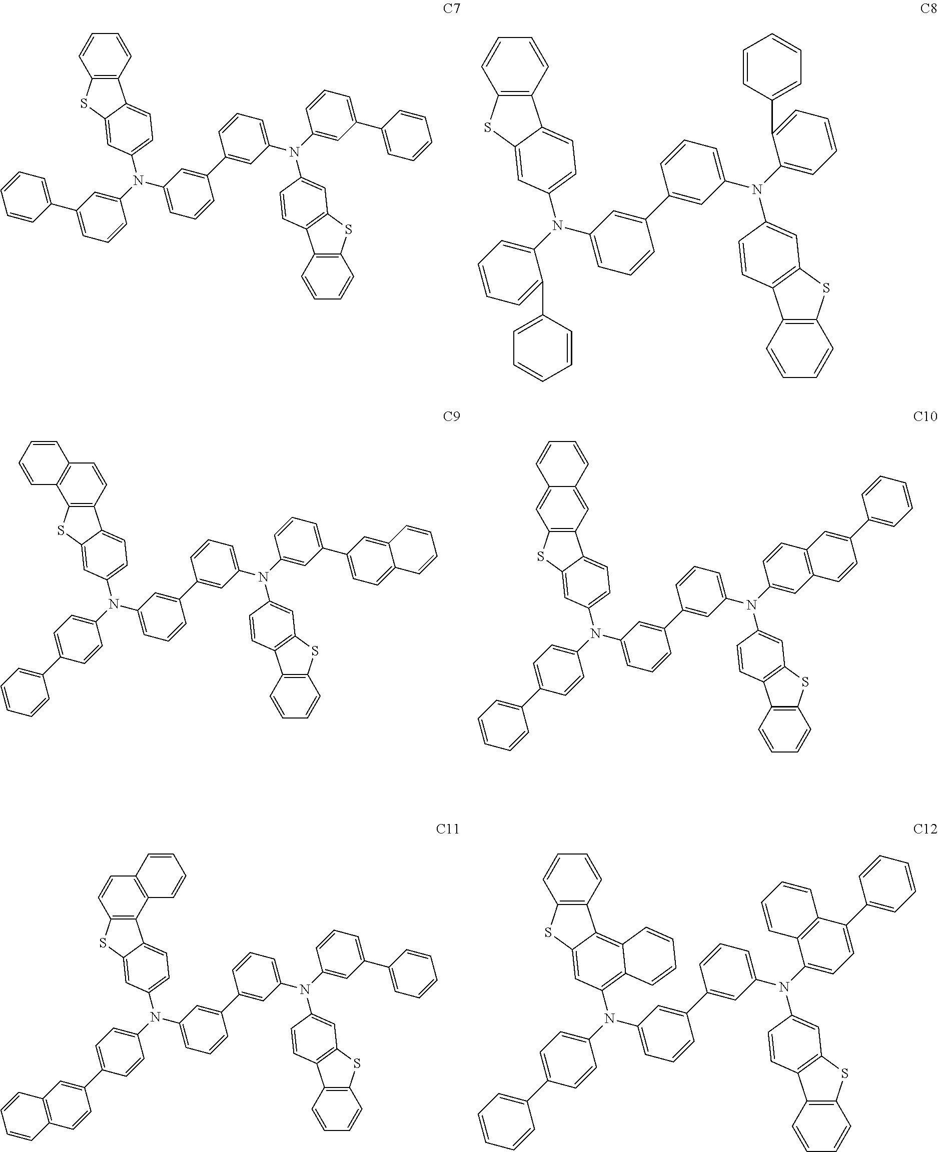

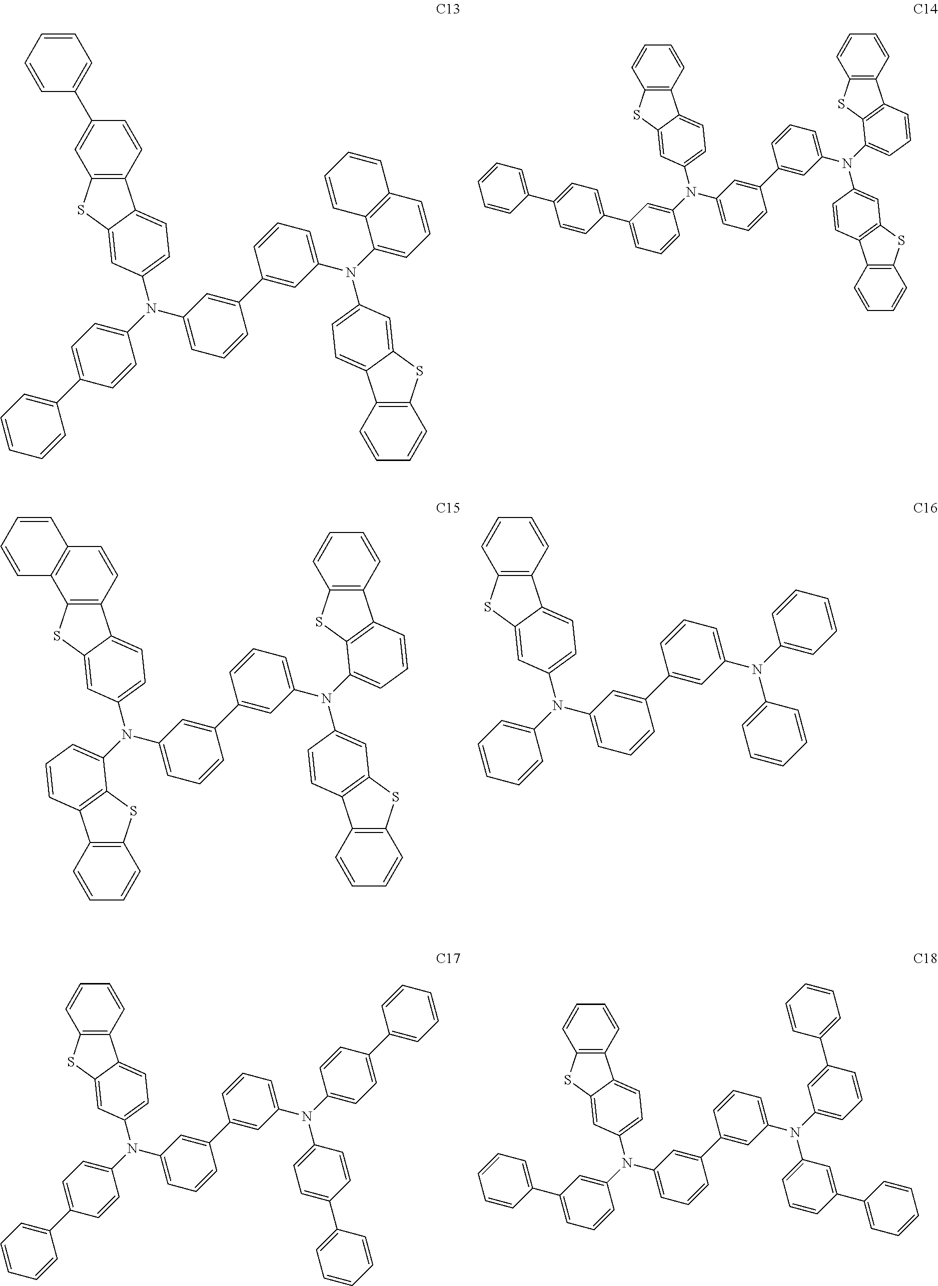

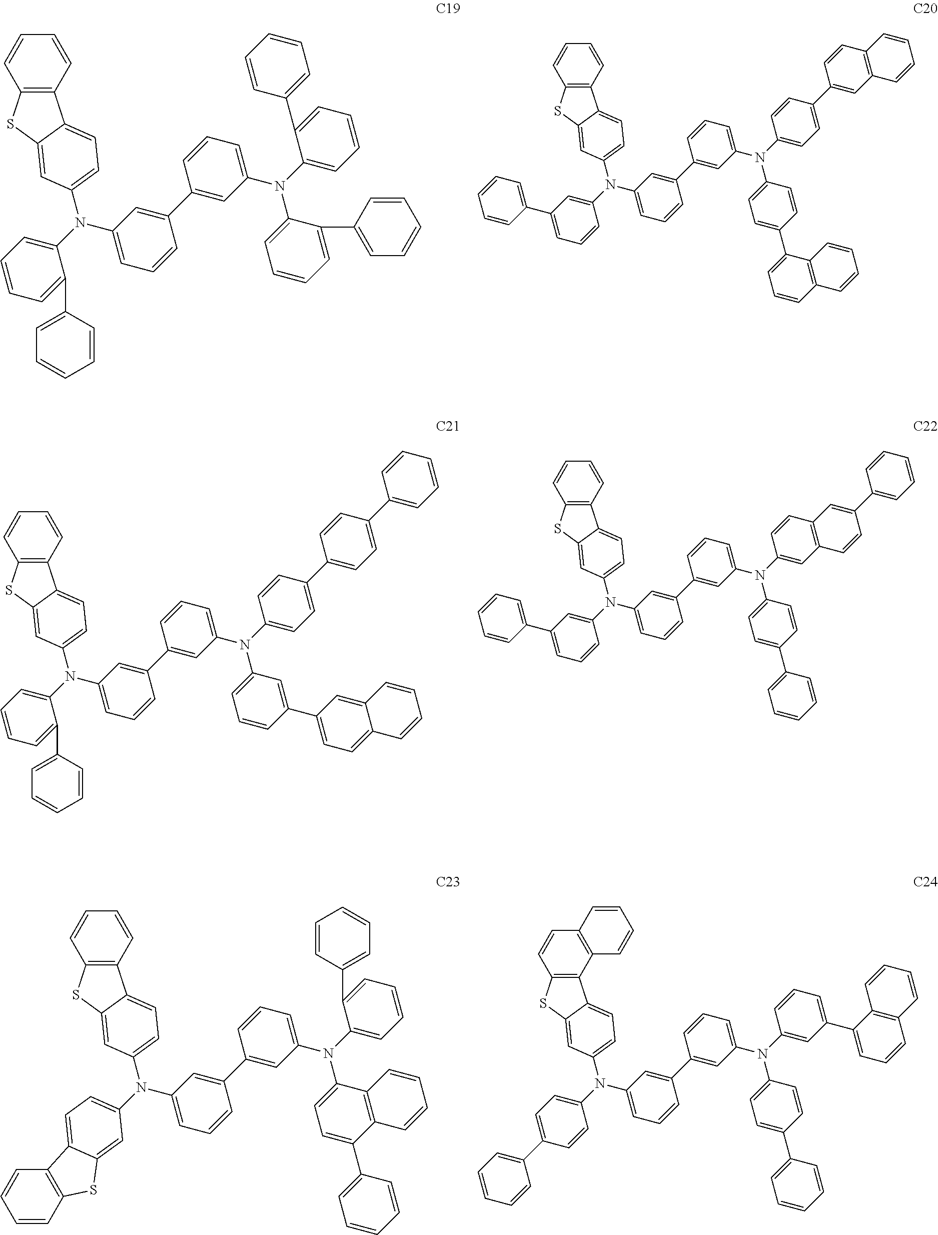

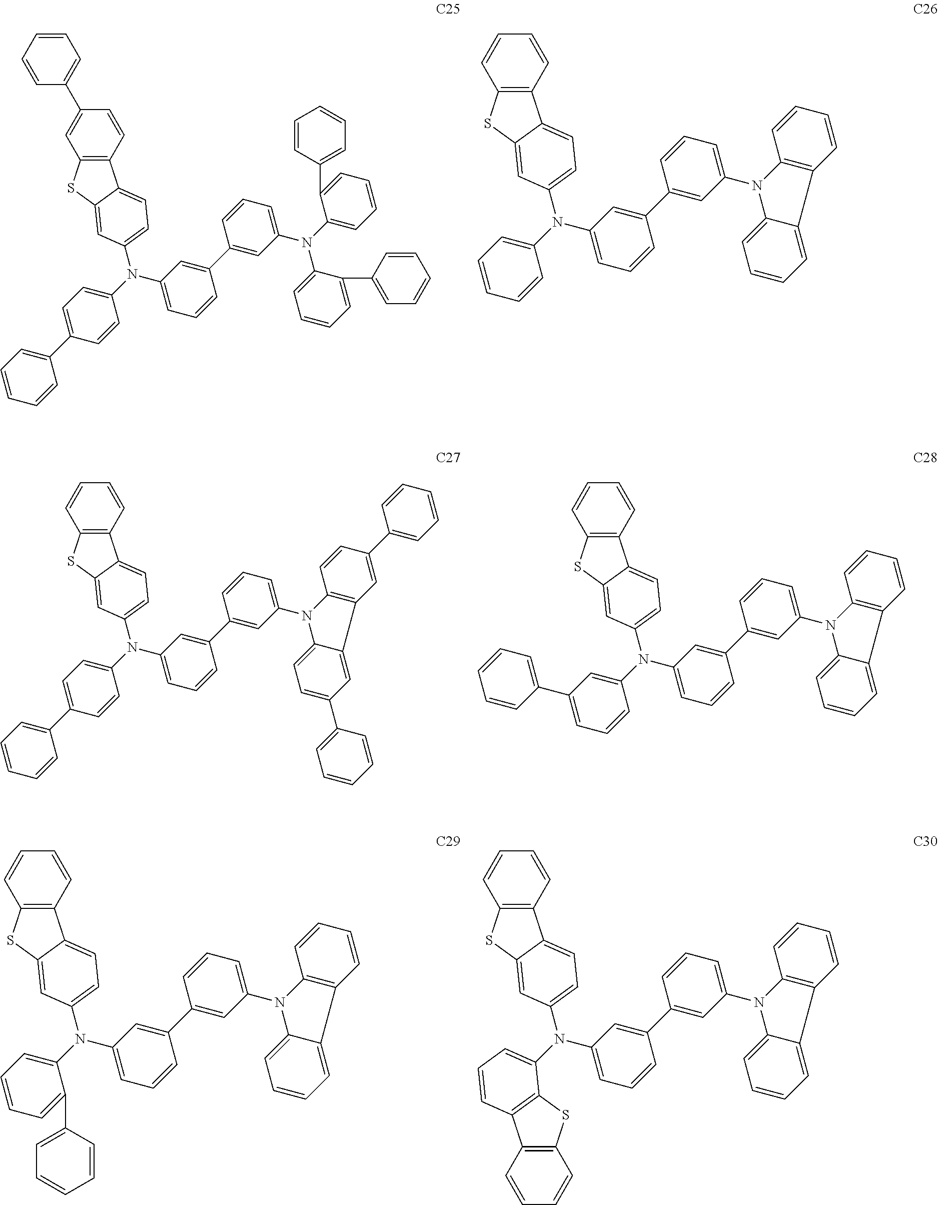

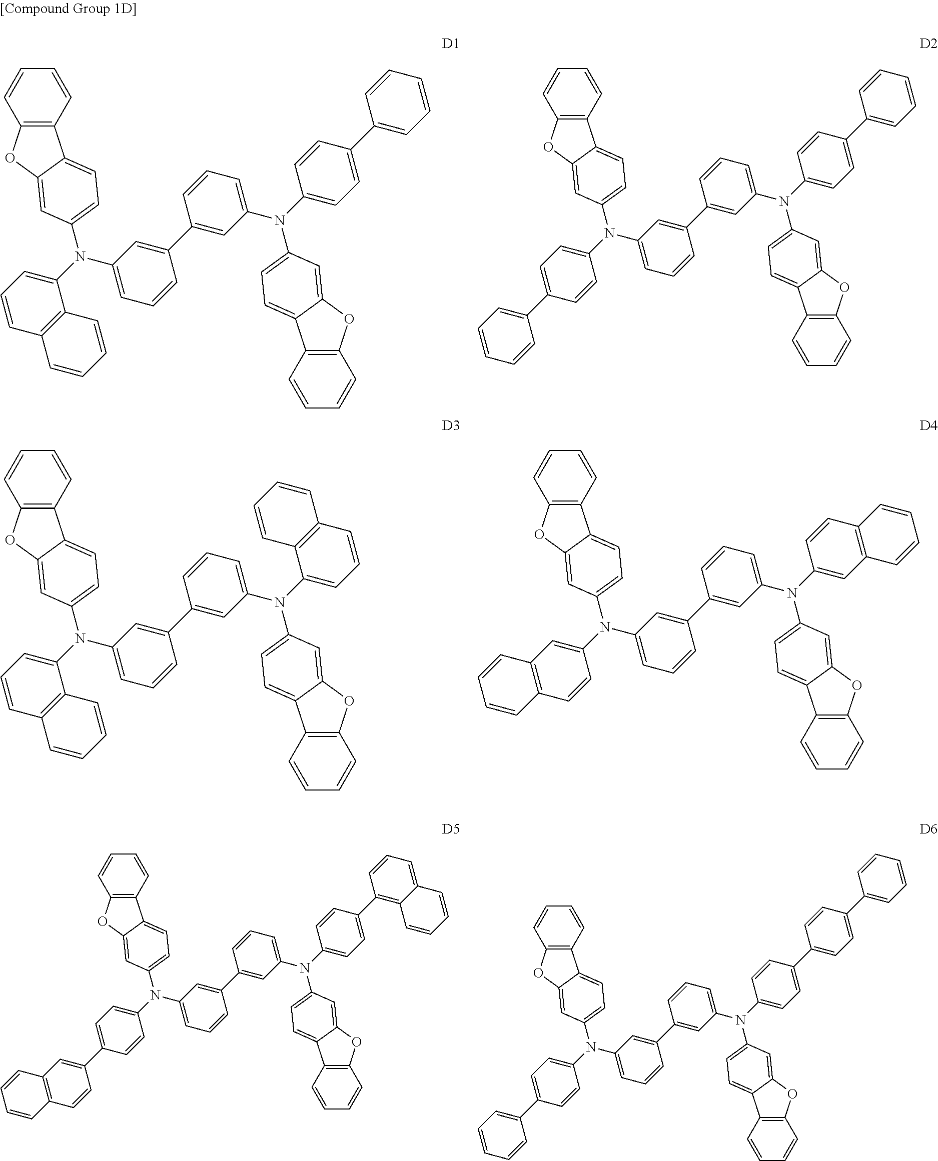

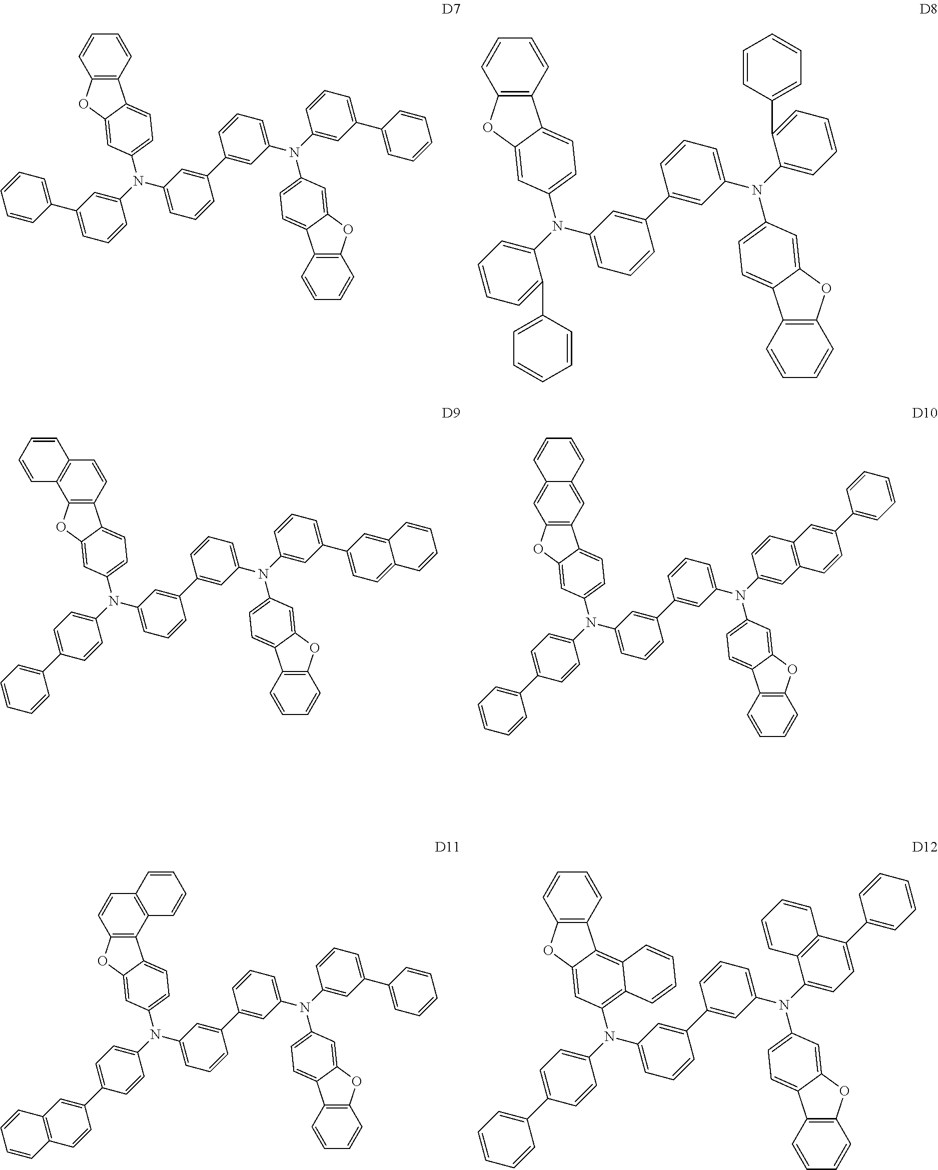

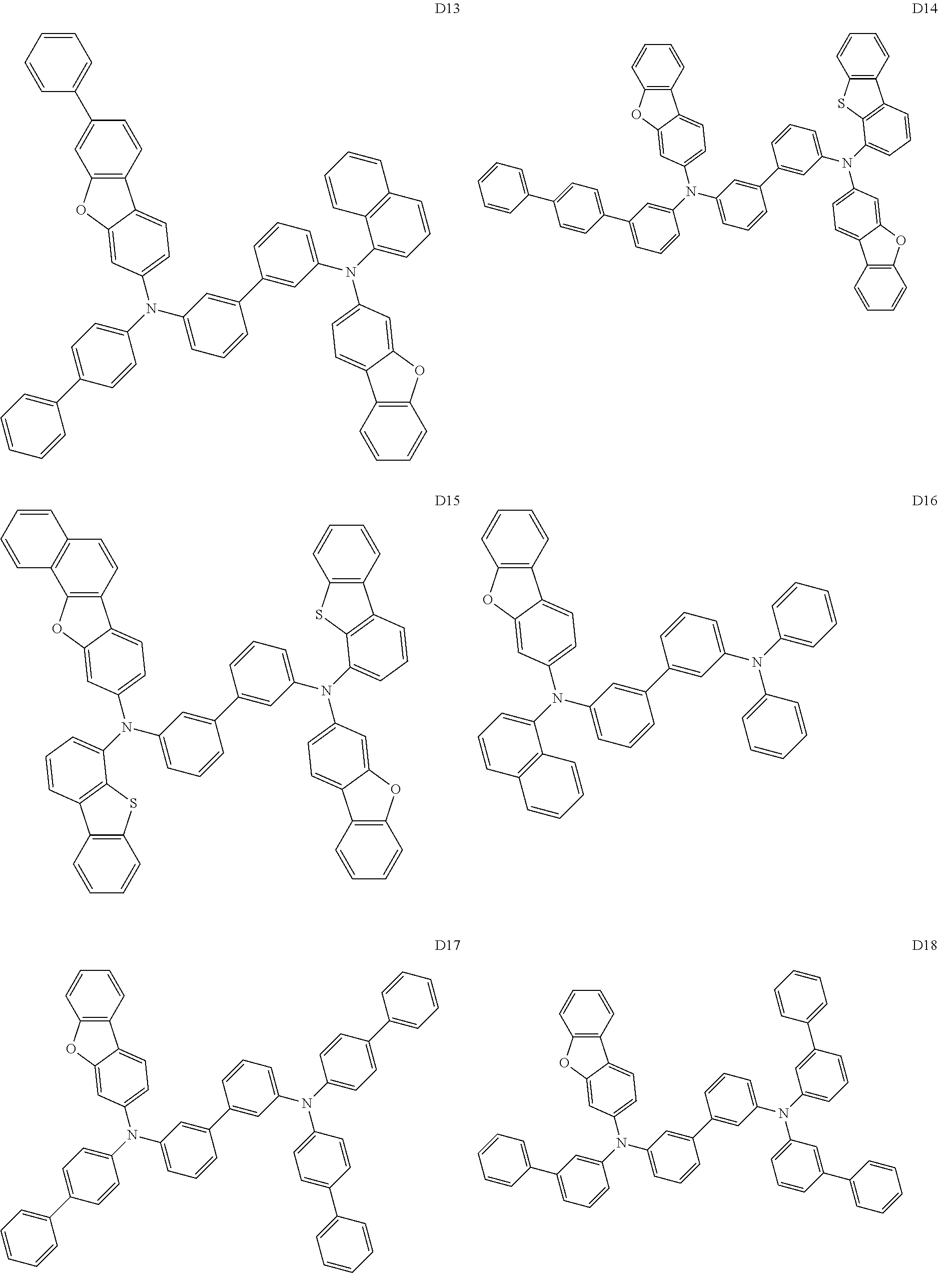

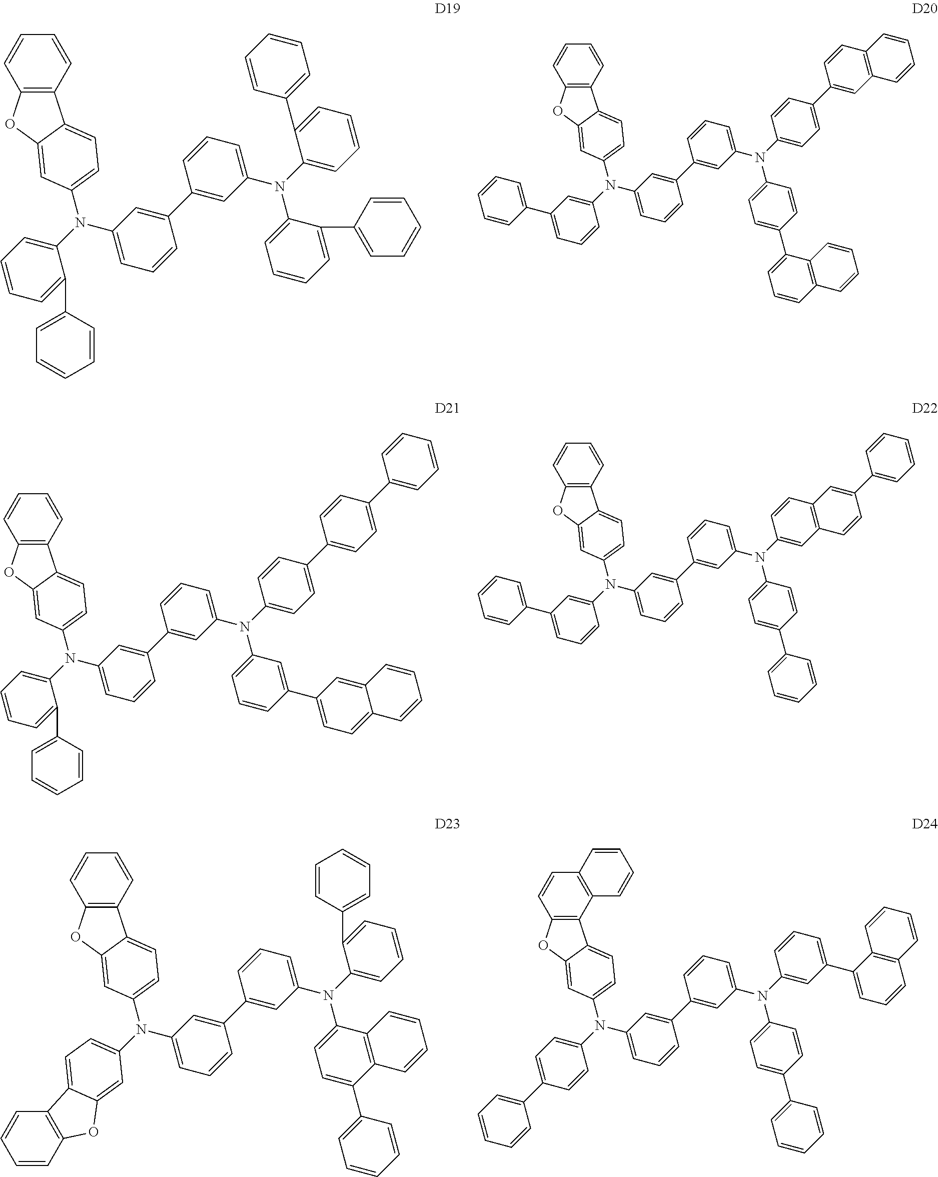

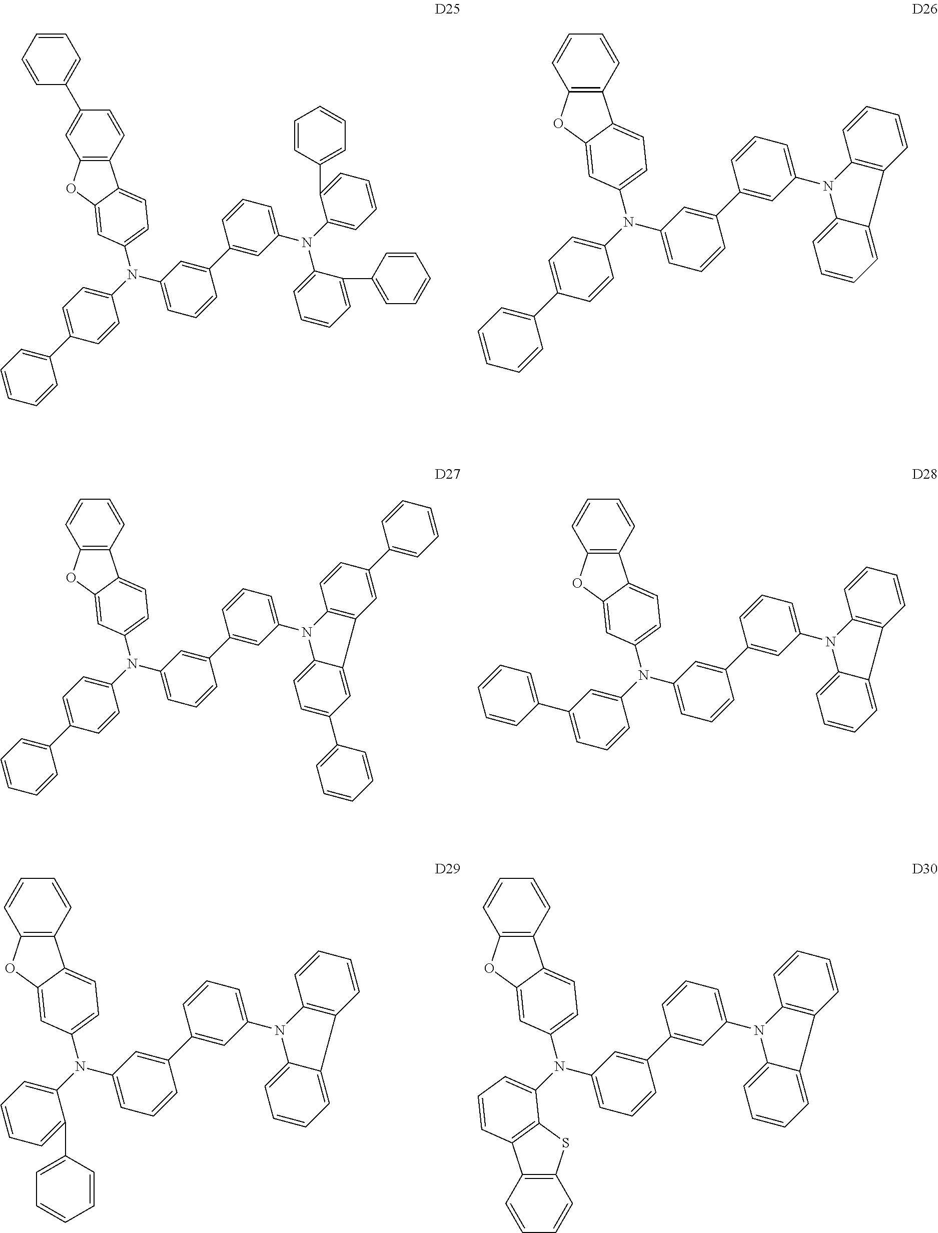

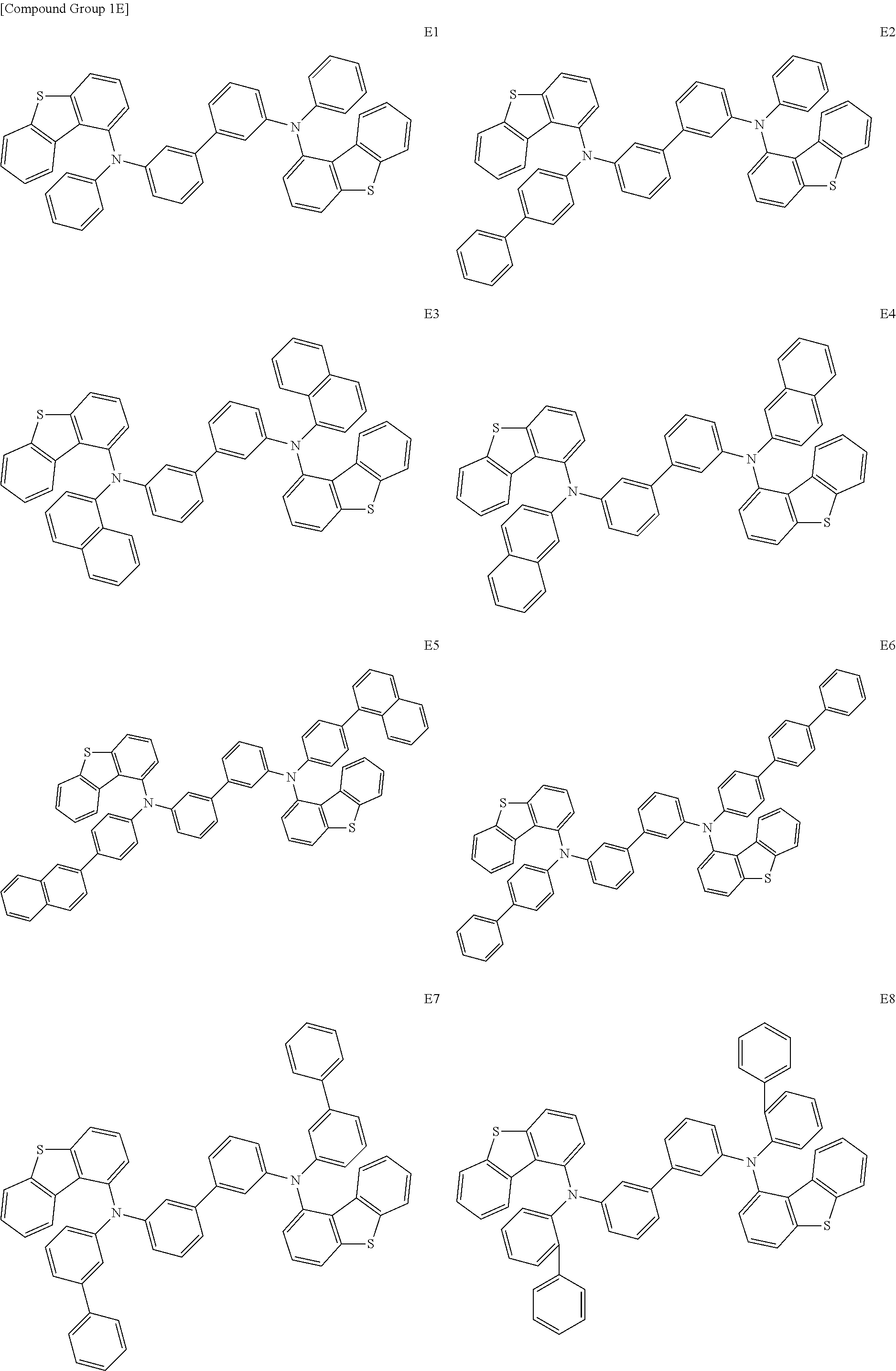

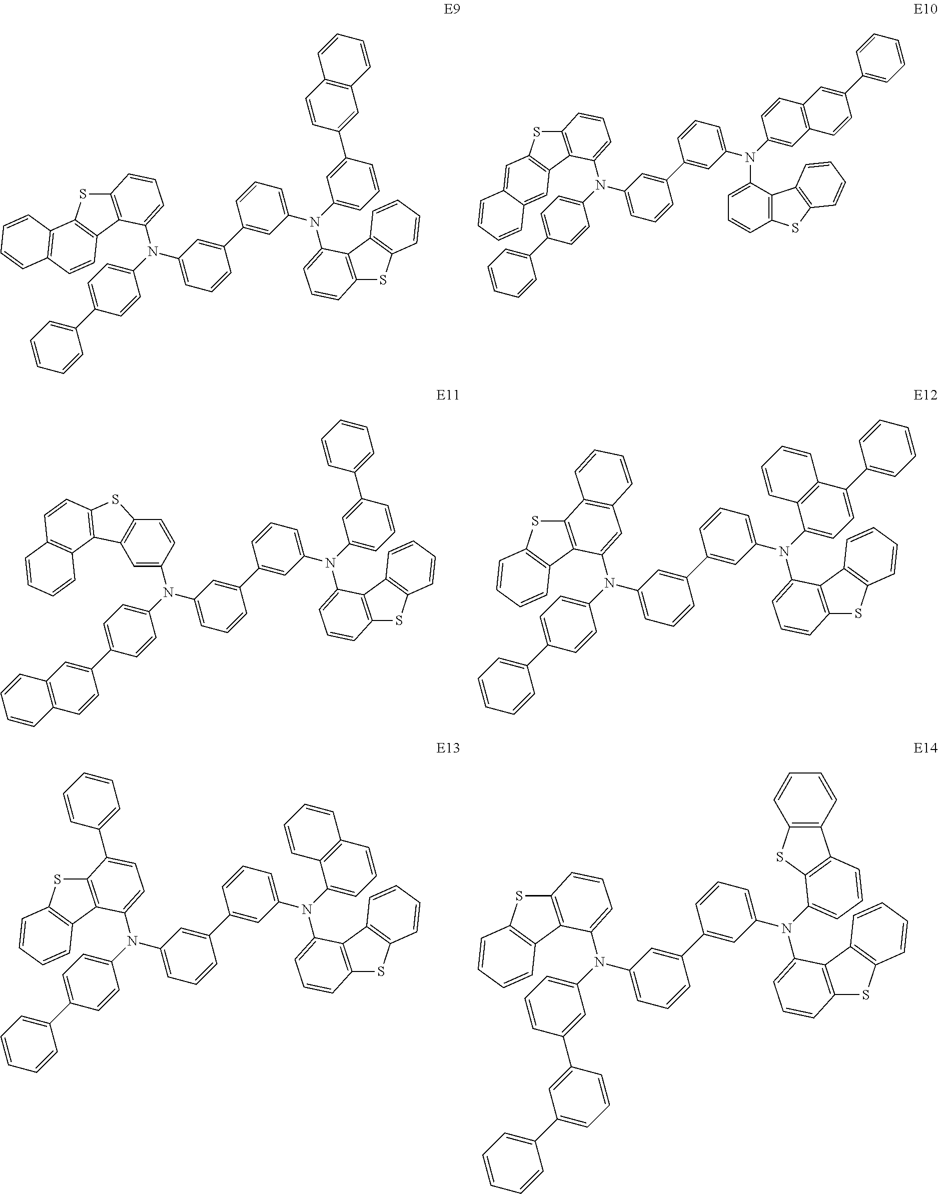

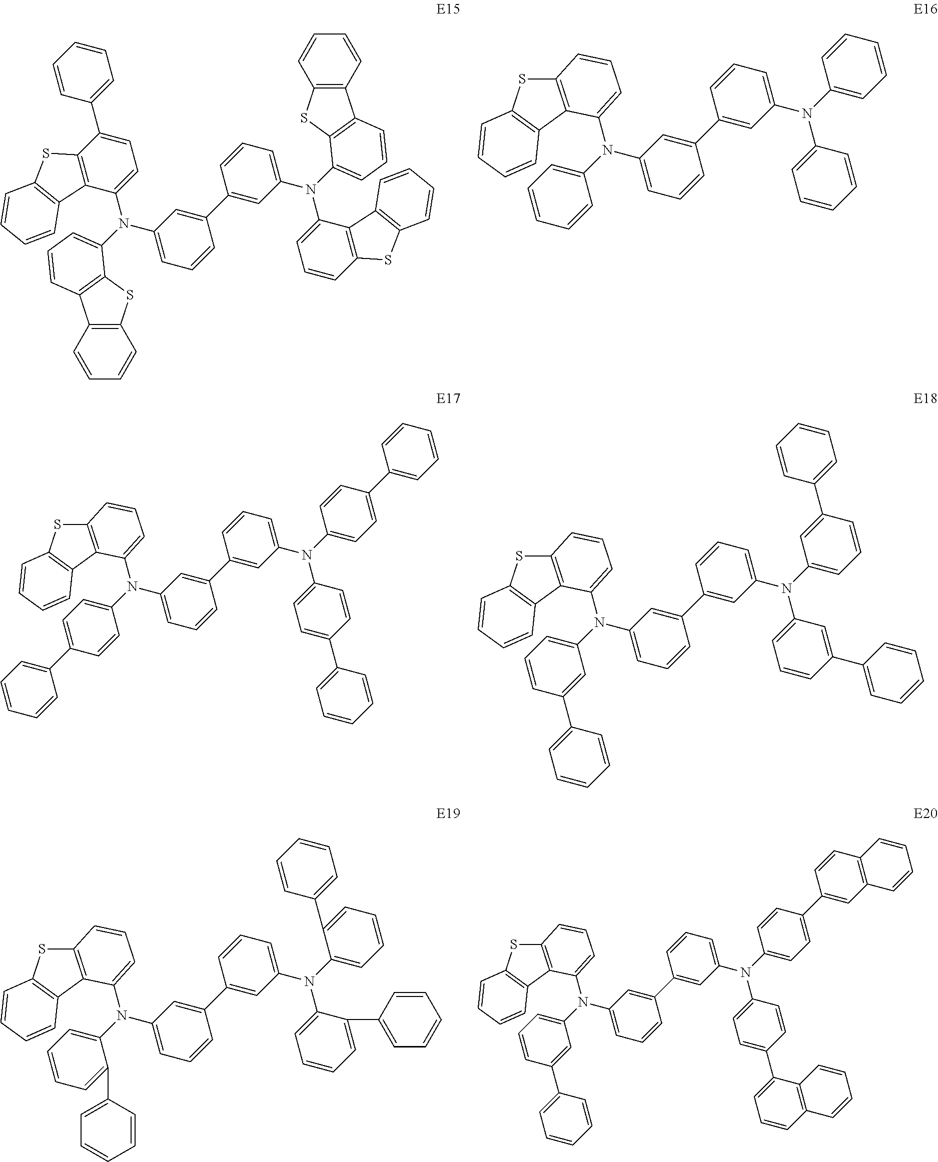

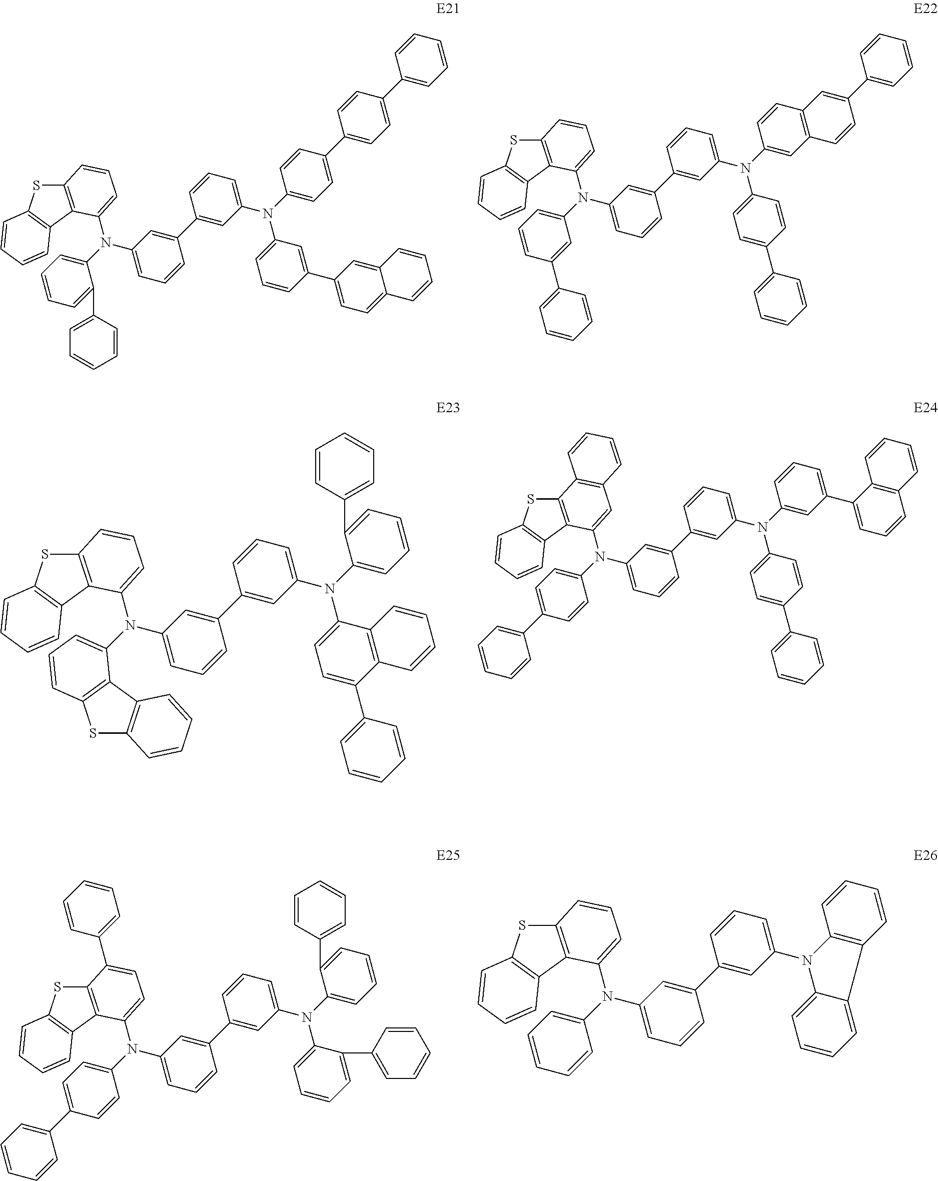

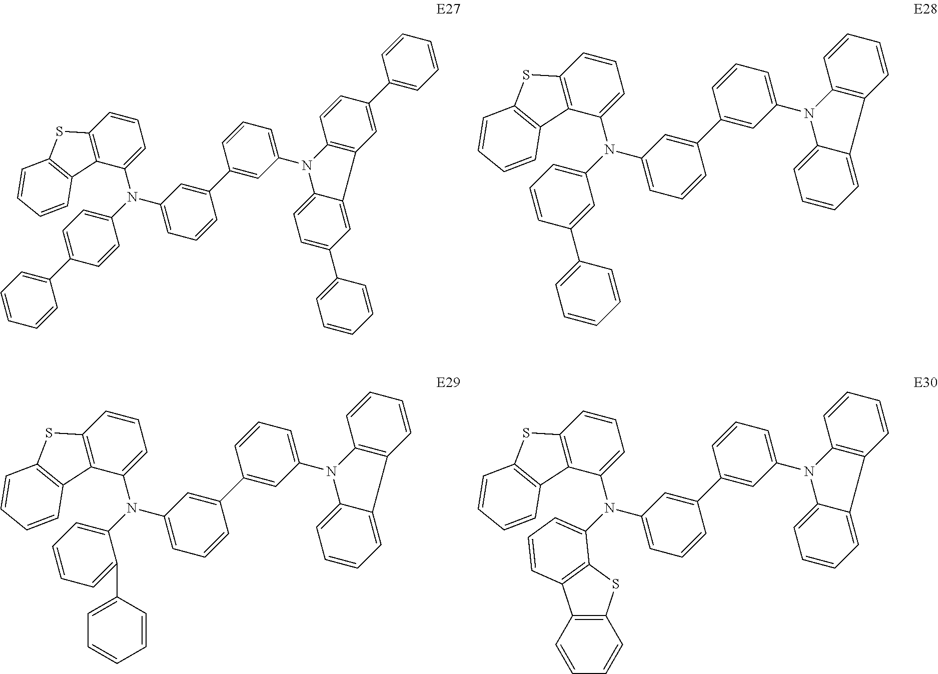 [SMILES: c1ccc(-c2ccc(N(c3cccc(-c4cccc(-n5c6ccc(-c7ccccc7)cc6c6cc(-c7ccccc7)ccc65)c4)c3)c3cccc4sc5ccccc5c34)cc2)cc1 ; c1ccc(-c2cccc(N(c3cccc(-c4cccc(-n5c6ccccc6c6ccccc65)c4)c3)c3cccc4sc5ccccc5c34)c2)cc1 ; c1ccc(-c2ccccc2N(c2cccc(-c3cccc(-n4c5ccccc5c5ccccc54)c3)c2)c2cccc3sc4ccccc4c23)cc1 ; c1cc(-c2cccc(-n3c4ccccc4c4ccccc43)c2)cc(N(c2cccc3c2sc2ccccc23)c2cccc3sc4ccccc4c23)c1]

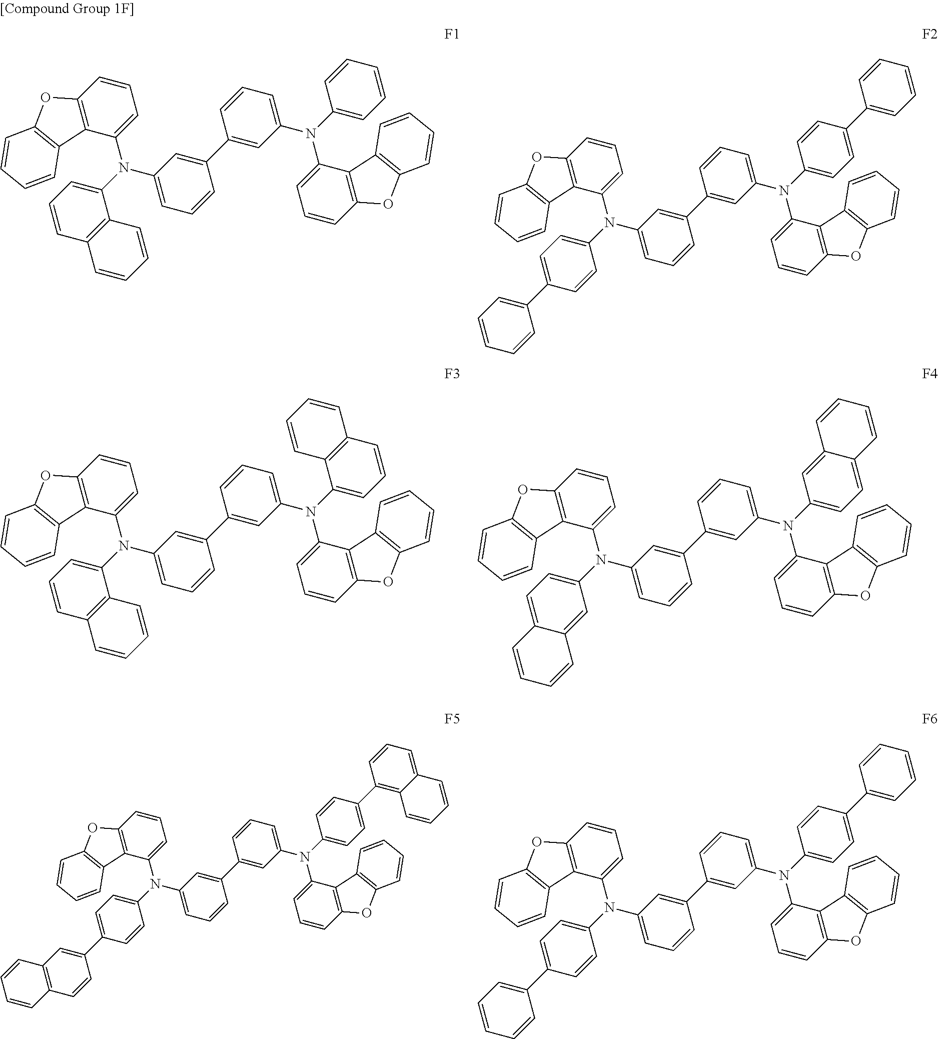

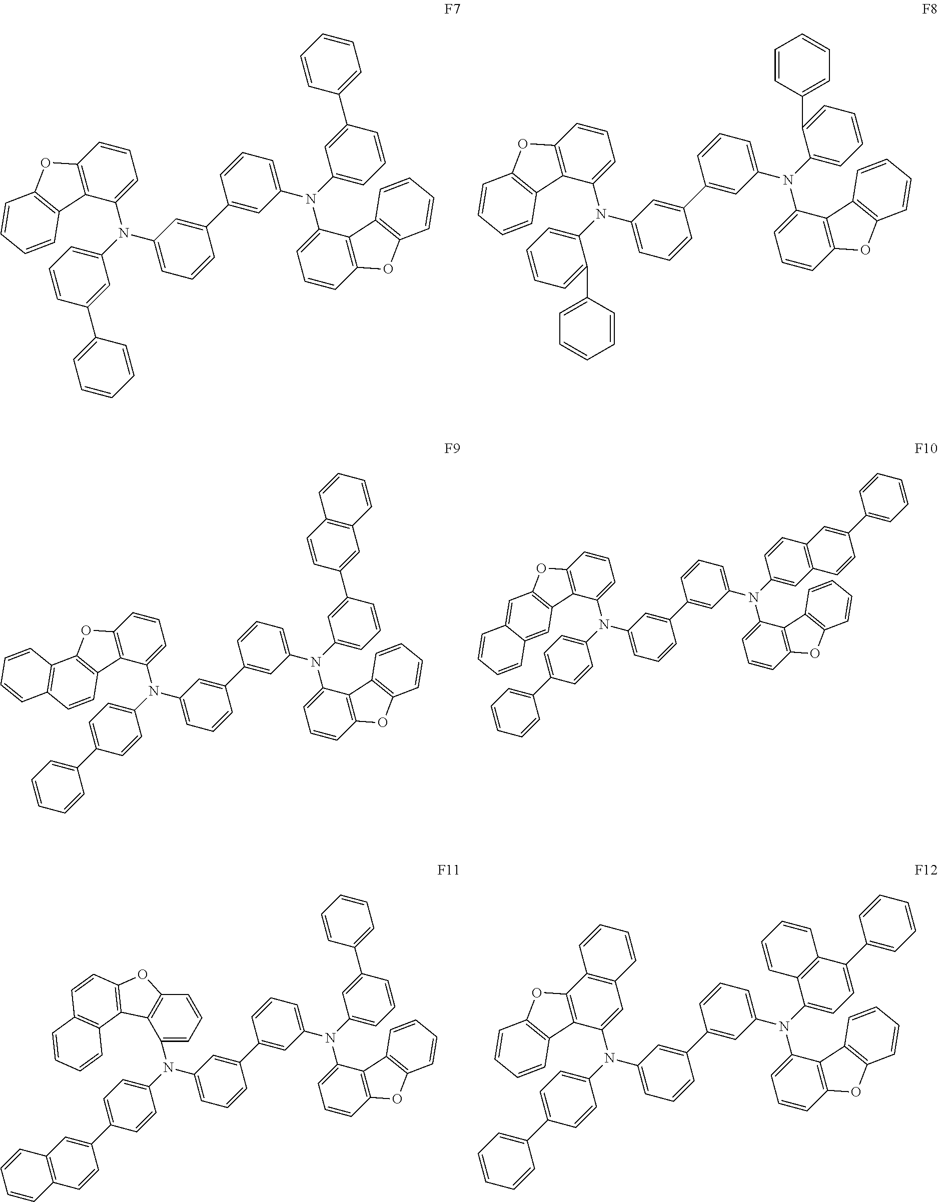

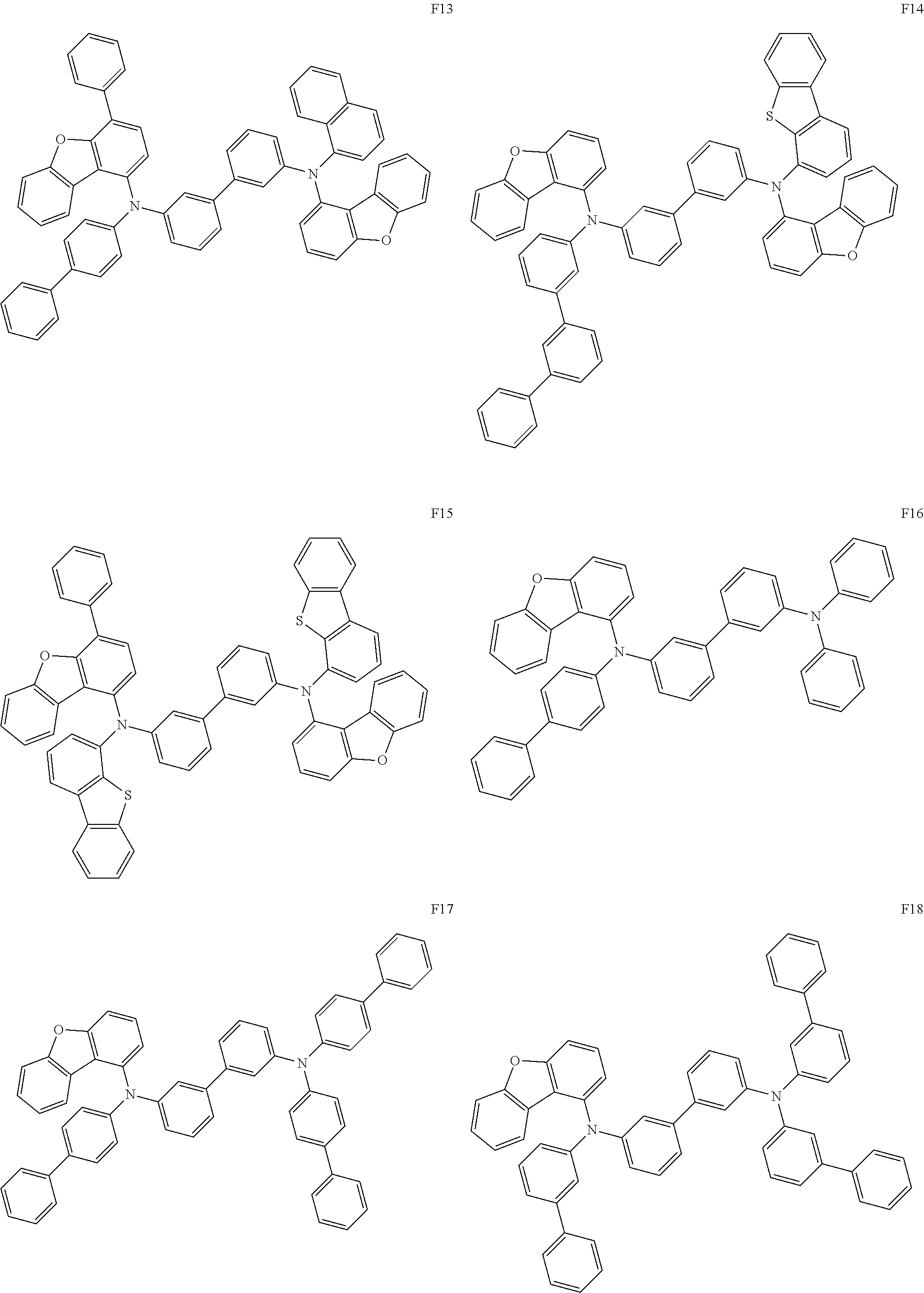

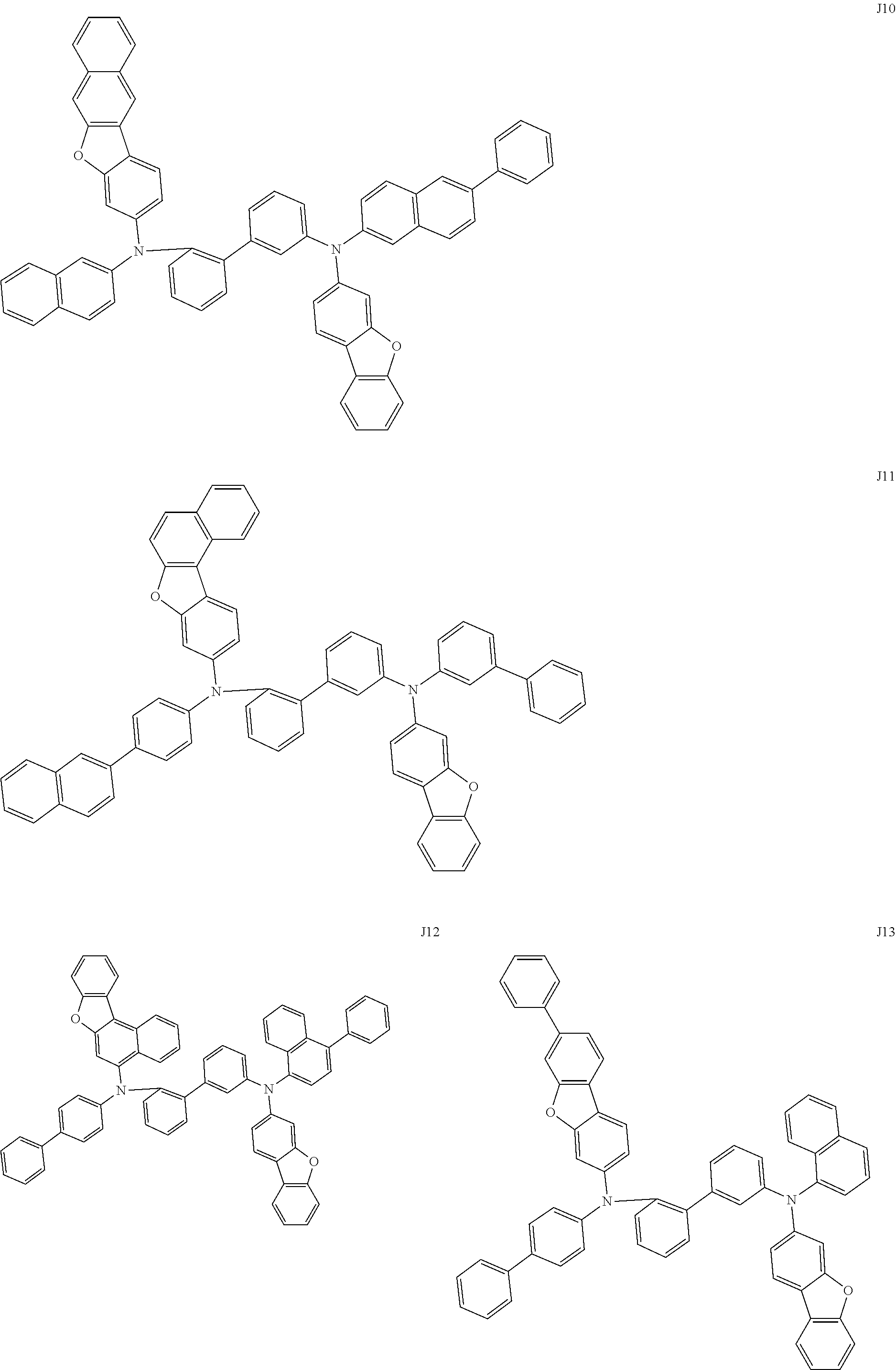 [SMILES: c1ccc(-c2ccc3cc(N(c4cccc(-c5ccccc5N(c5ccc6ccccc6c5)c5ccc6c(c5)oc5cc7ccccc7cc56)c4)c4ccc5c(c4)oc4ccccc45)ccc3c2)cc1 ; c1ccc(-c2cccc(N(c3cccc(-c4ccccc4N(c4ccc(-c5ccc6ccccc6c5)cc4)c4ccc5c(c4)oc4ccc6ccccc6c45)c3)c3ccc4c(c3)oc3ccccc34)c2)cc1 ; c1ccc(-c2ccc(N(c3ccccc3-c3cccc(N(c4ccc5c(c4)oc4ccccc45)c4ccc(-c5ccccc5)c5ccccc45)c3)c3cc4oc5ccccc5c4c4ccccc34)cc2)cc1 ; c1ccc(-c2ccc(N(c3ccc4c(c3)oc3cc(-c5ccccc5)ccc34)c3ccccc3-c3cccc(N(c4ccc5c(c4)oc4ccccc45)c4cccc5ccccc45)c3)cc2)cc1]

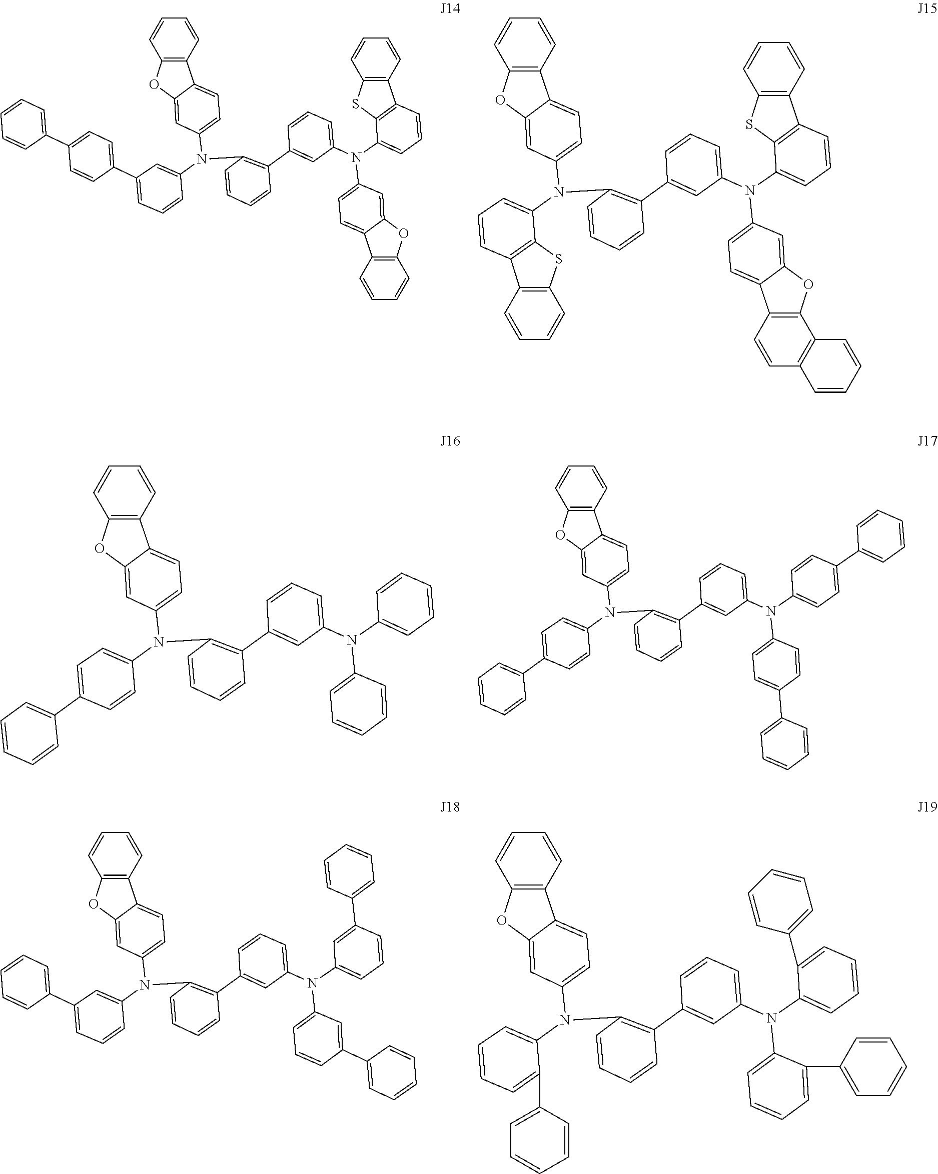

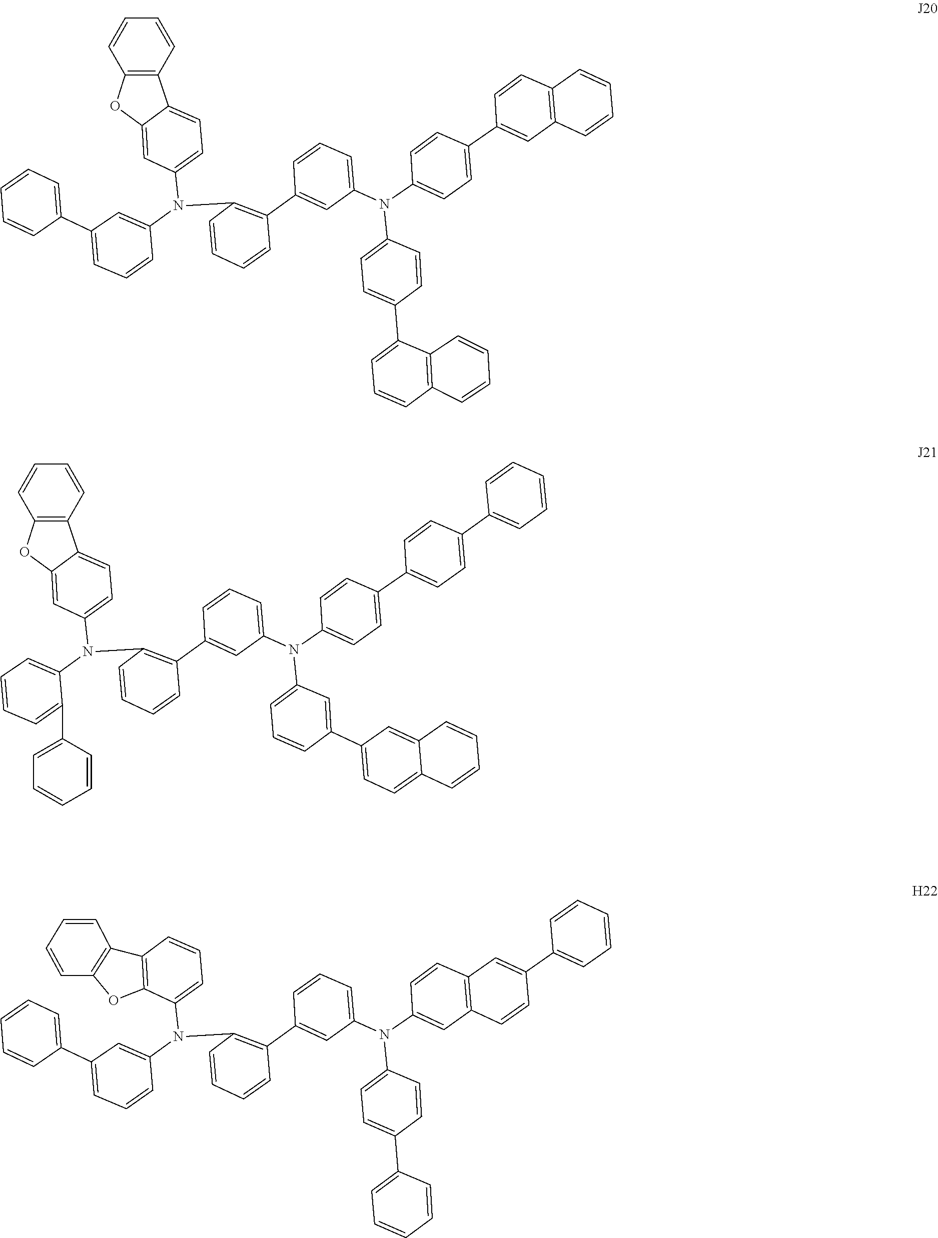 [SMILES: c1ccc(-c2cccc(N(c3ccc4c(c3)oc3ccccc34)c3ccccc3-c3cccc(N(c4ccc(-c5ccc6ccccc6c5)cc4)c4ccc(-c5cccc6ccccc56)cc4)c3)c2)cc1 ; c1ccc(-c2ccc(-c3ccc(N(c4cccc(-c5ccc6ccccc6c5)c4)c4cccc(-c5ccccc5N(c5ccc6c(c5)oc5ccccc56)c5ccccc5-c5ccccc5)c4)cc3)cc2)cc1 ; c1ccc(-c2ccc(N(c3cccc(-c4ccccc4N(c4cccc(-c5ccccc5)c4)c4cccc5c4oc4ccccc45)c3)c3ccc4cc(-c5ccccc5)ccc4c3)cc2)cc1]

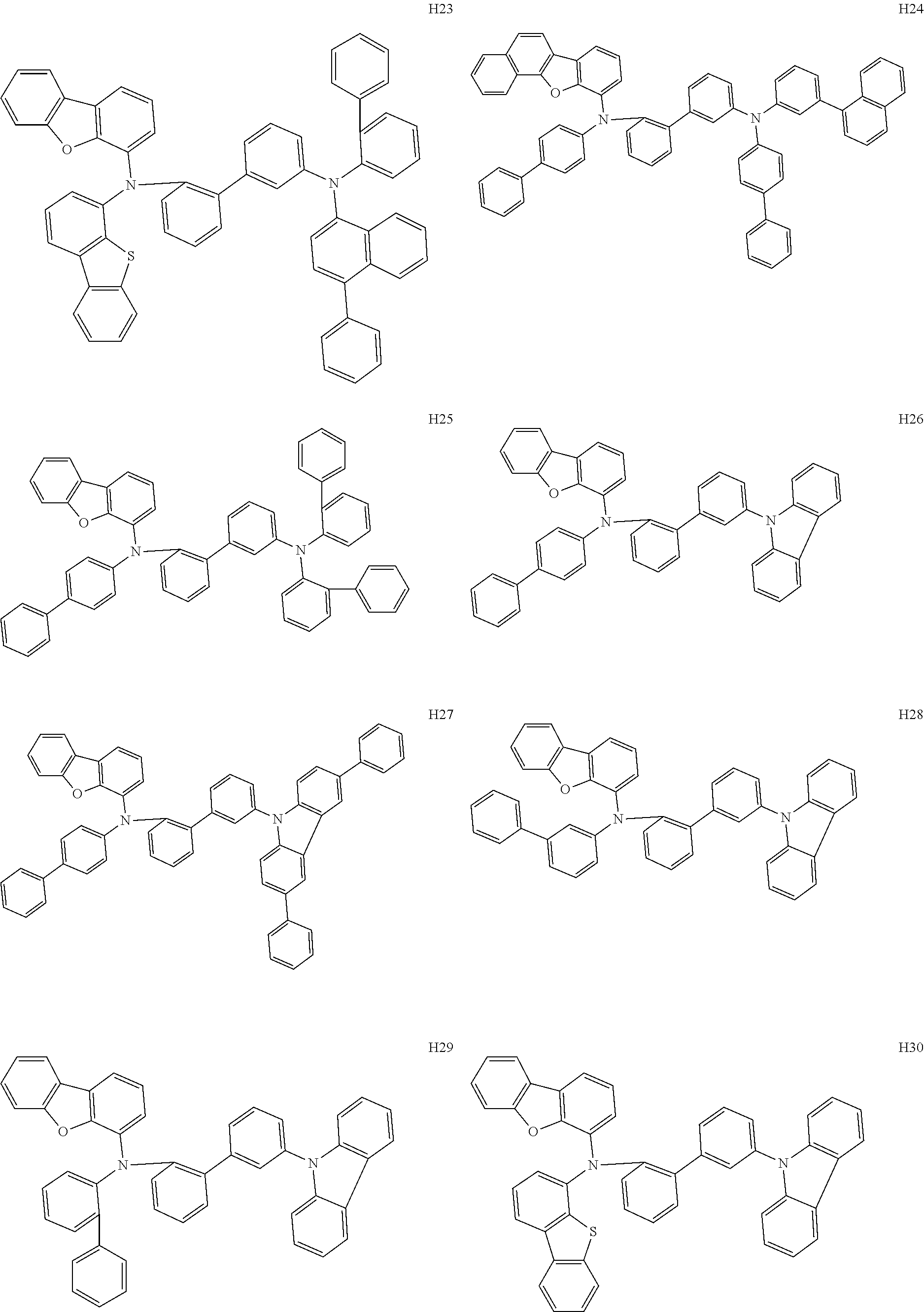

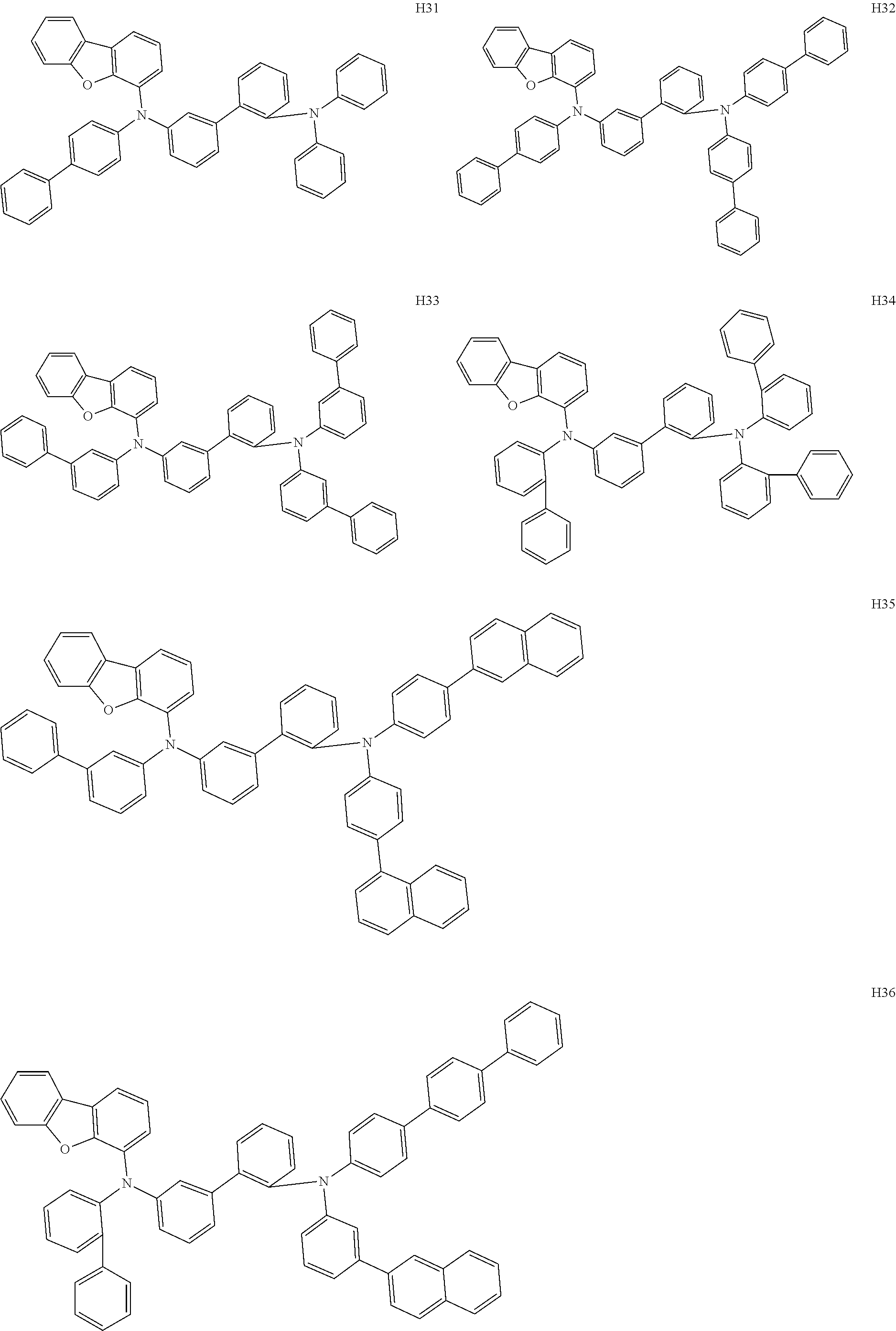

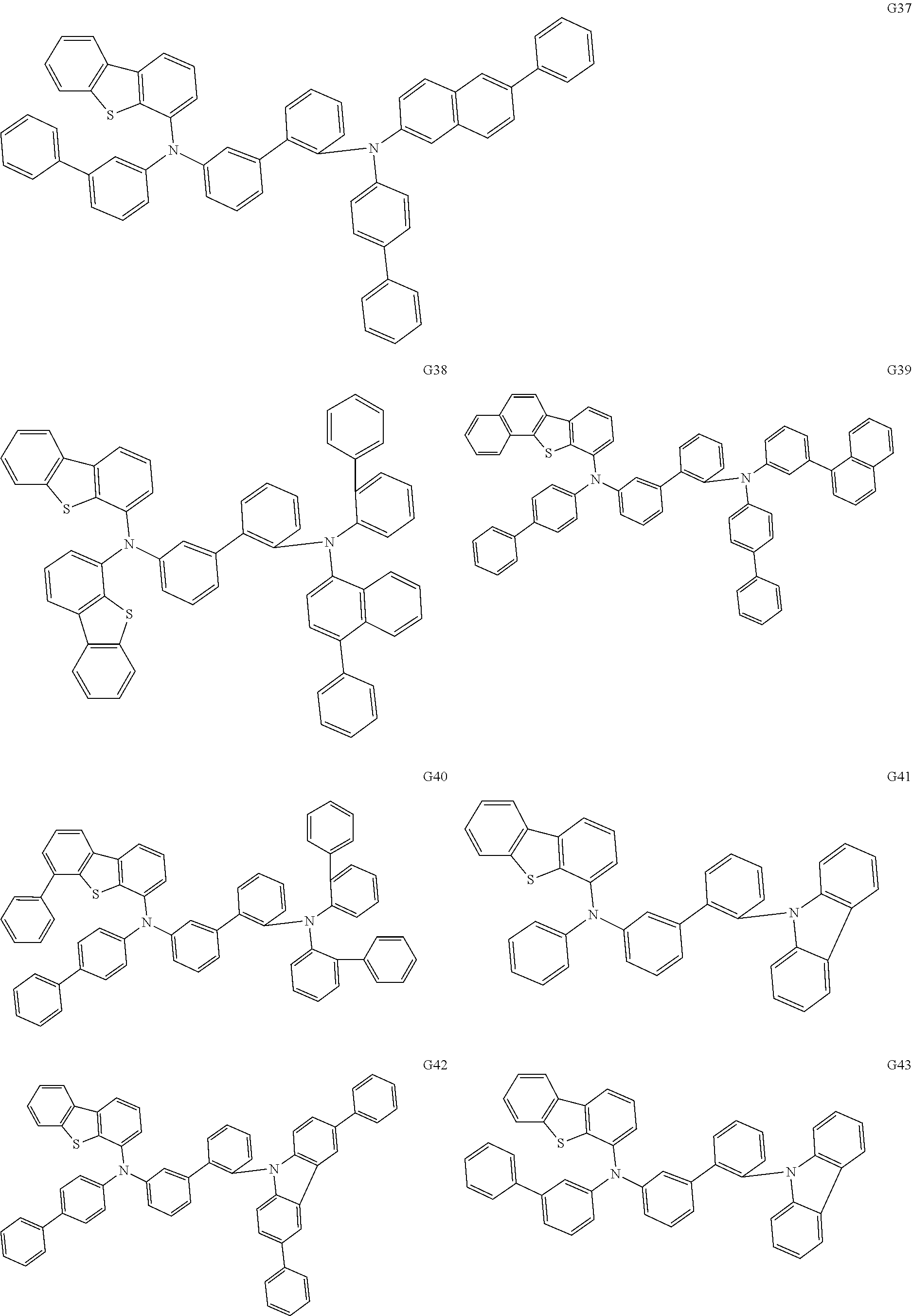

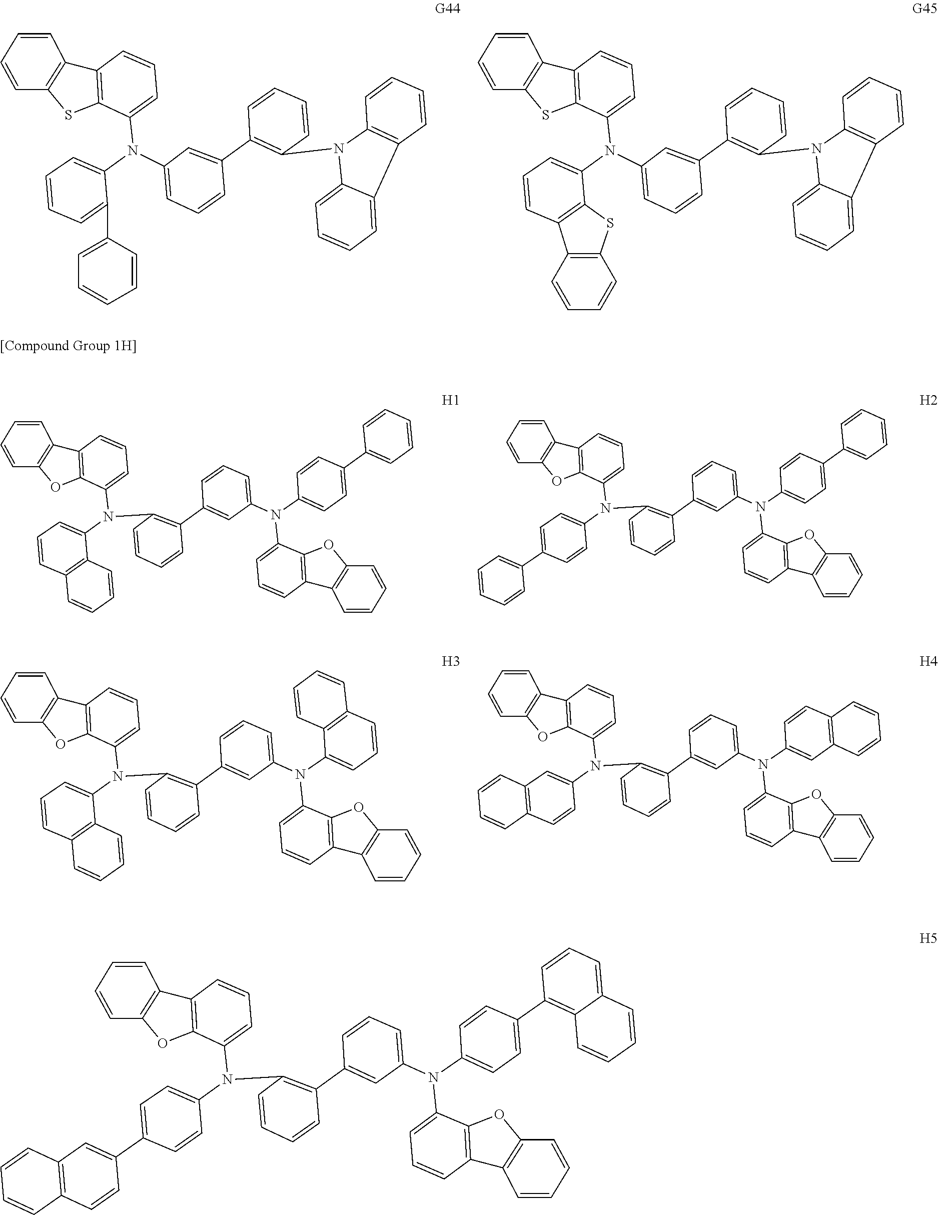

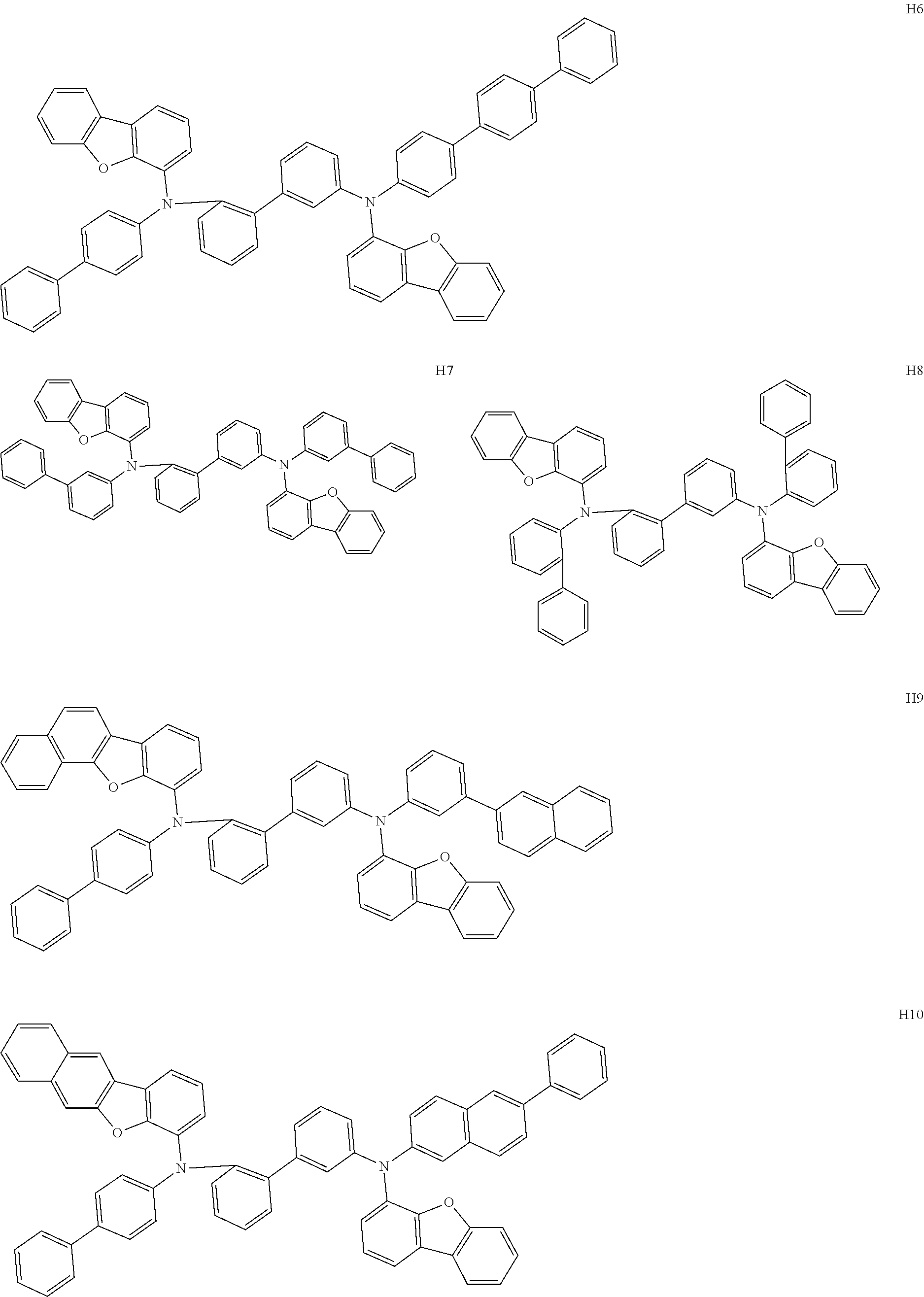

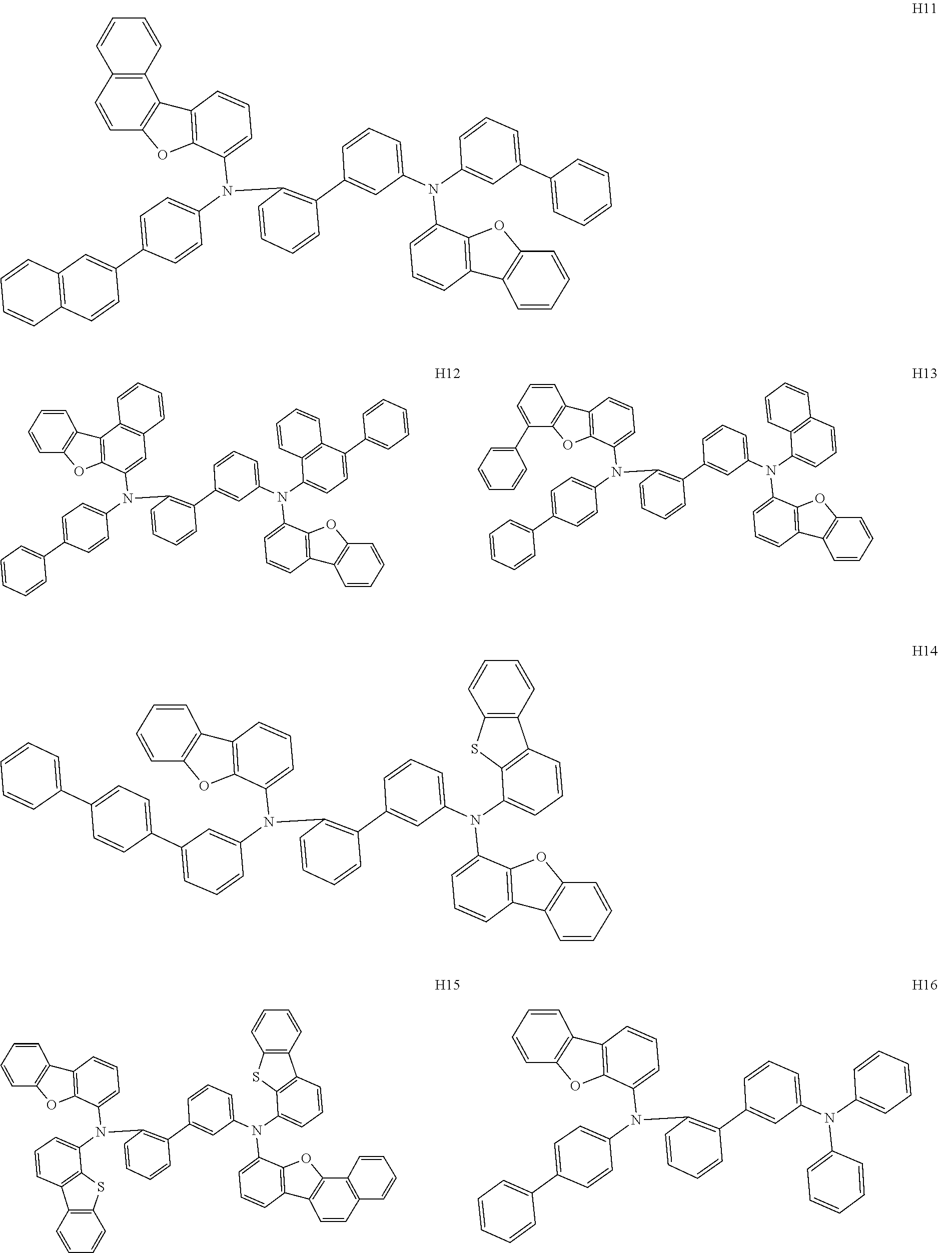

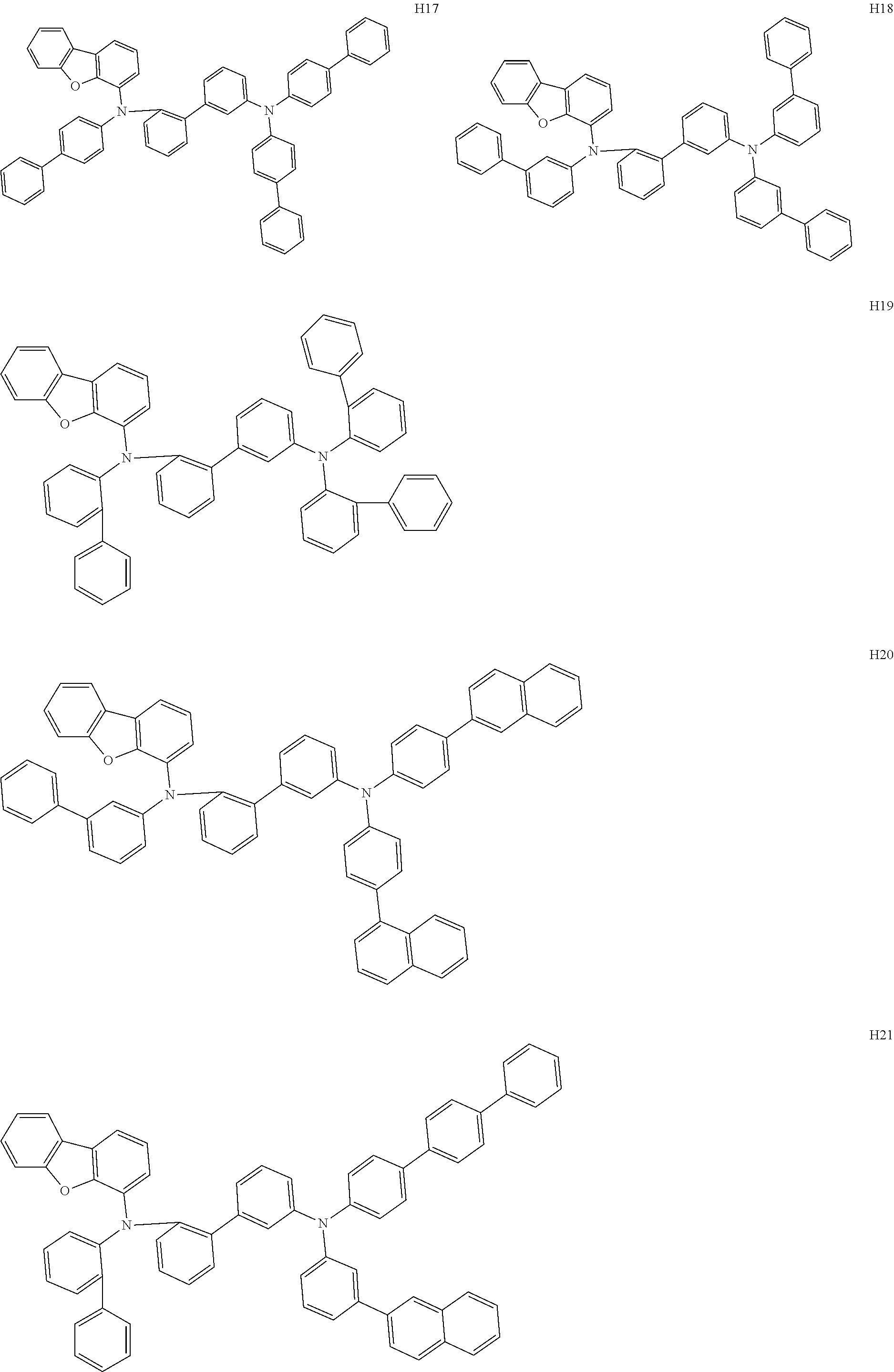

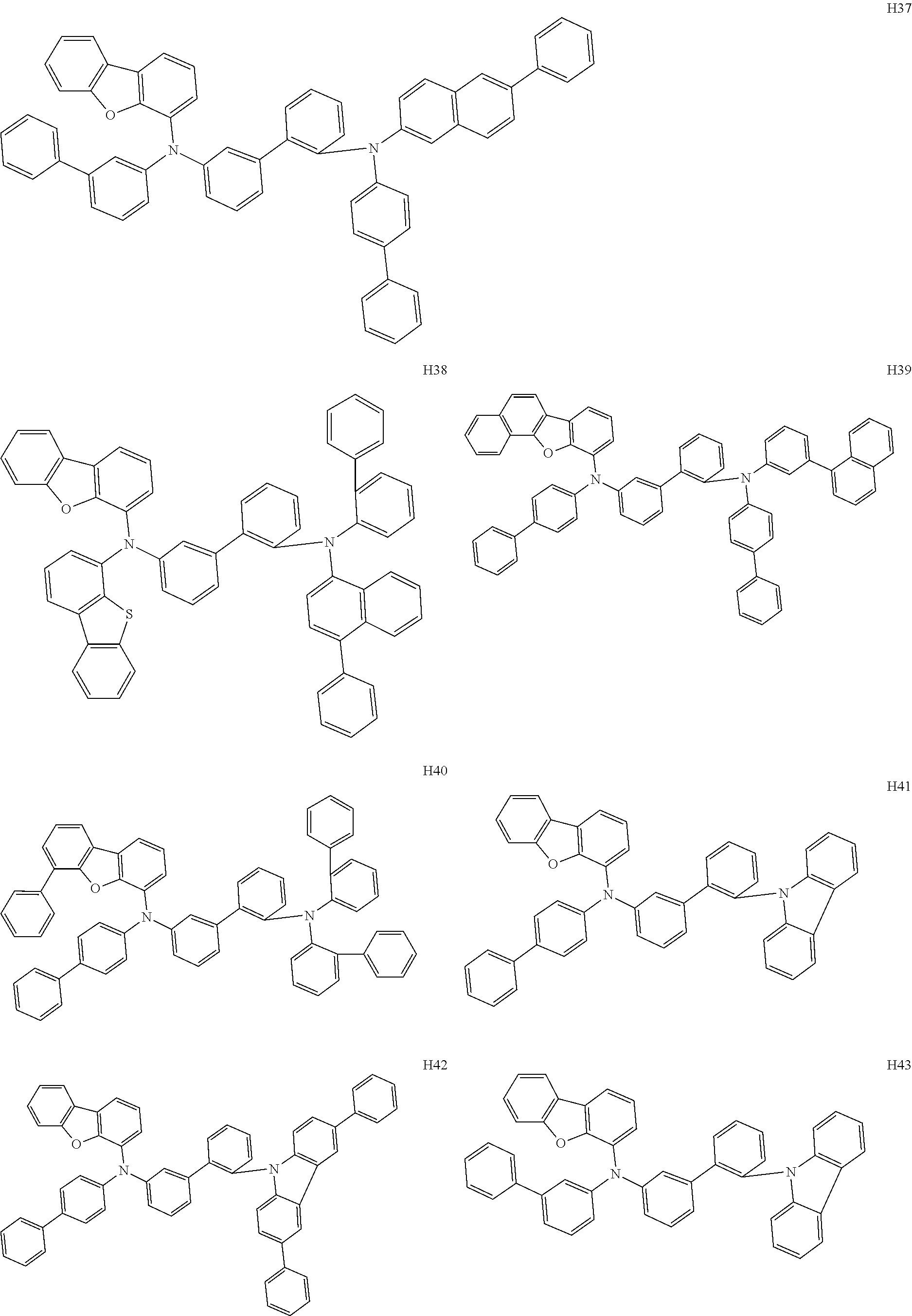

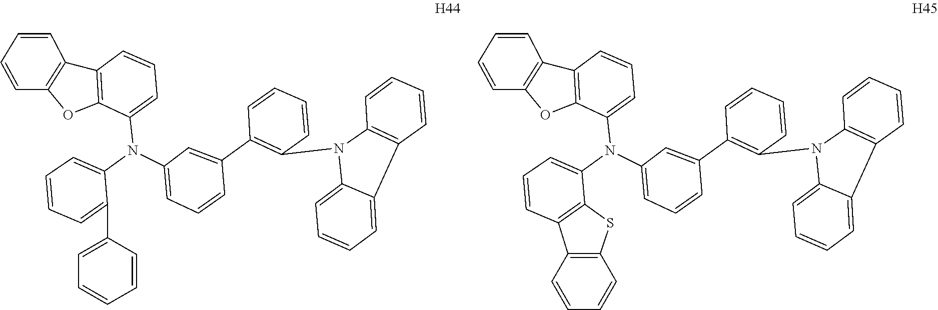 [SMILES: c1ccc(-c2ccccc2N(c2cccc(-c3ccccc3-n3c4ccccc4c4ccccc43)c2)c2cccc3c2oc2ccccc23)cc1 ; c1cc(-c2ccccc2-n2c3ccccc3c3ccccc32)cc(N(c2cccc3c2oc2ccccc23)c2cccc3c2sc2ccccc23)c1]

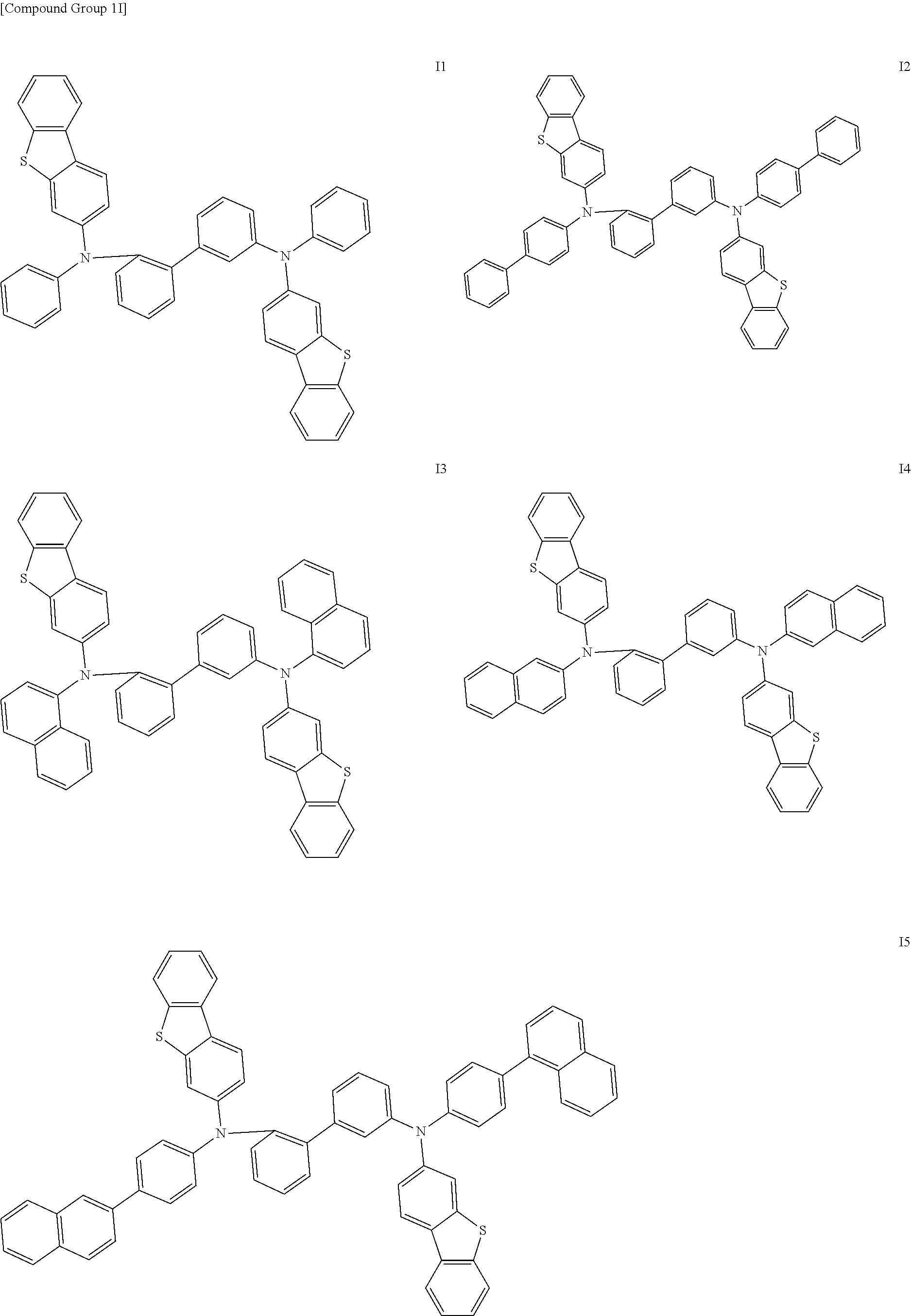

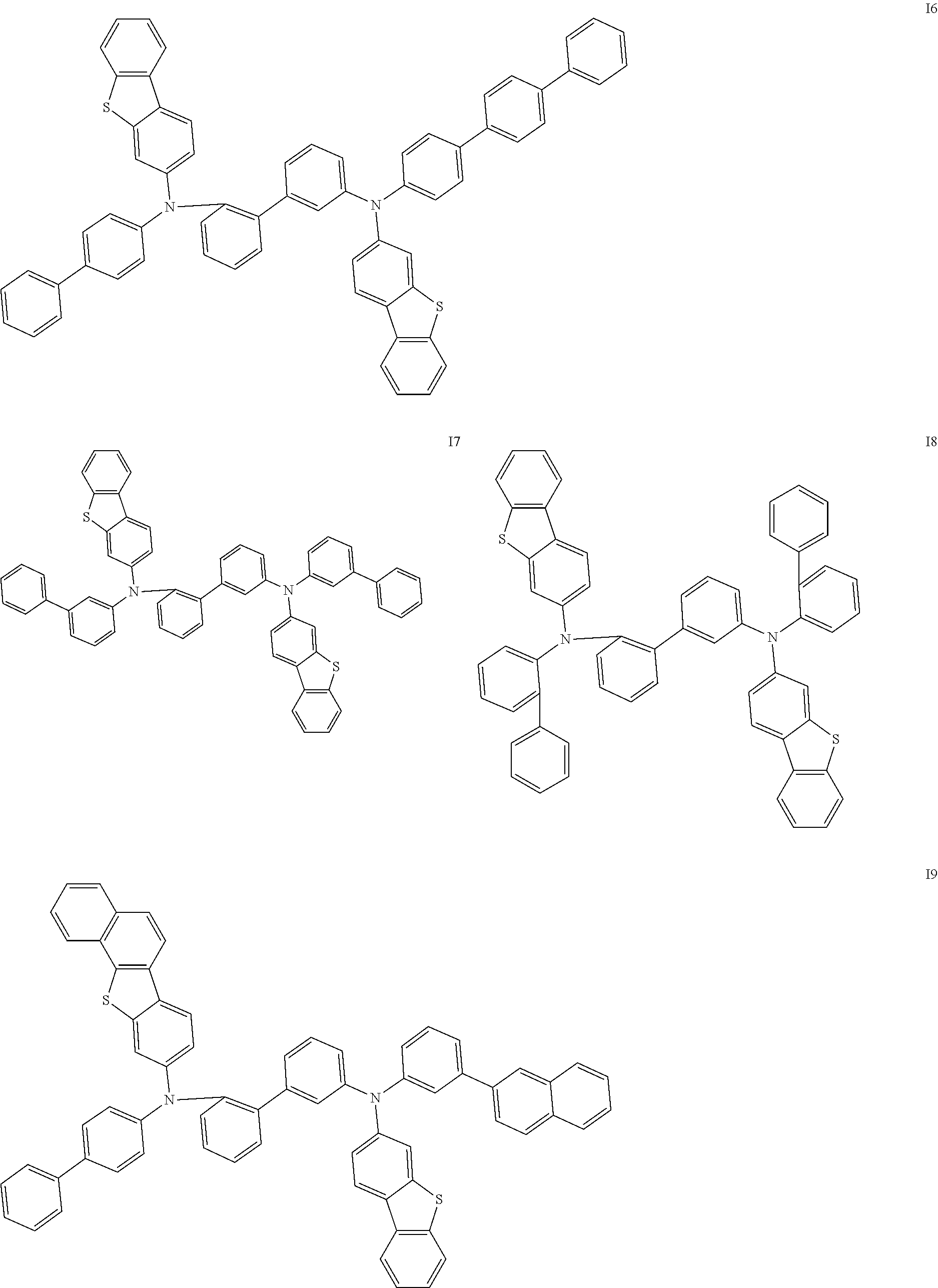 [SMILES: c1ccc(-c2ccc(-c3ccc(N(c4cccc(-c5ccccc5N(c5ccc(-c6ccccc6)cc5)c5ccc6c(c5)sc5ccccc56)c4)c4ccc5c(c4)sc4ccccc45)cc3)cc2)cc1 ; c1ccc(-c2cccc(N(c3cccc(-c4ccccc4N(c4cccc(-c5ccccc5)c4)c4ccc5c(c4)sc4ccccc45)c3)c3ccc4c(c3)sc3ccccc34)c2)cc1 ; c1ccc(-c2ccccc2N(c2cccc(-c3ccccc3N(c3ccc4c(c3)sc3ccccc34)c3ccccc3-c3ccccc3)c2)c2ccc3c(c2)sc2ccccc23)cc1 ; c1ccc(-c2ccc(N(c3ccc4c(c3)sc3c5ccccc5ccc43)c3ccccc3-c3cccc(N(c4cccc(-c5ccc6ccccc6c5)c4)c4ccc5c(c4)sc4ccccc45)c3)cc2)cc1]

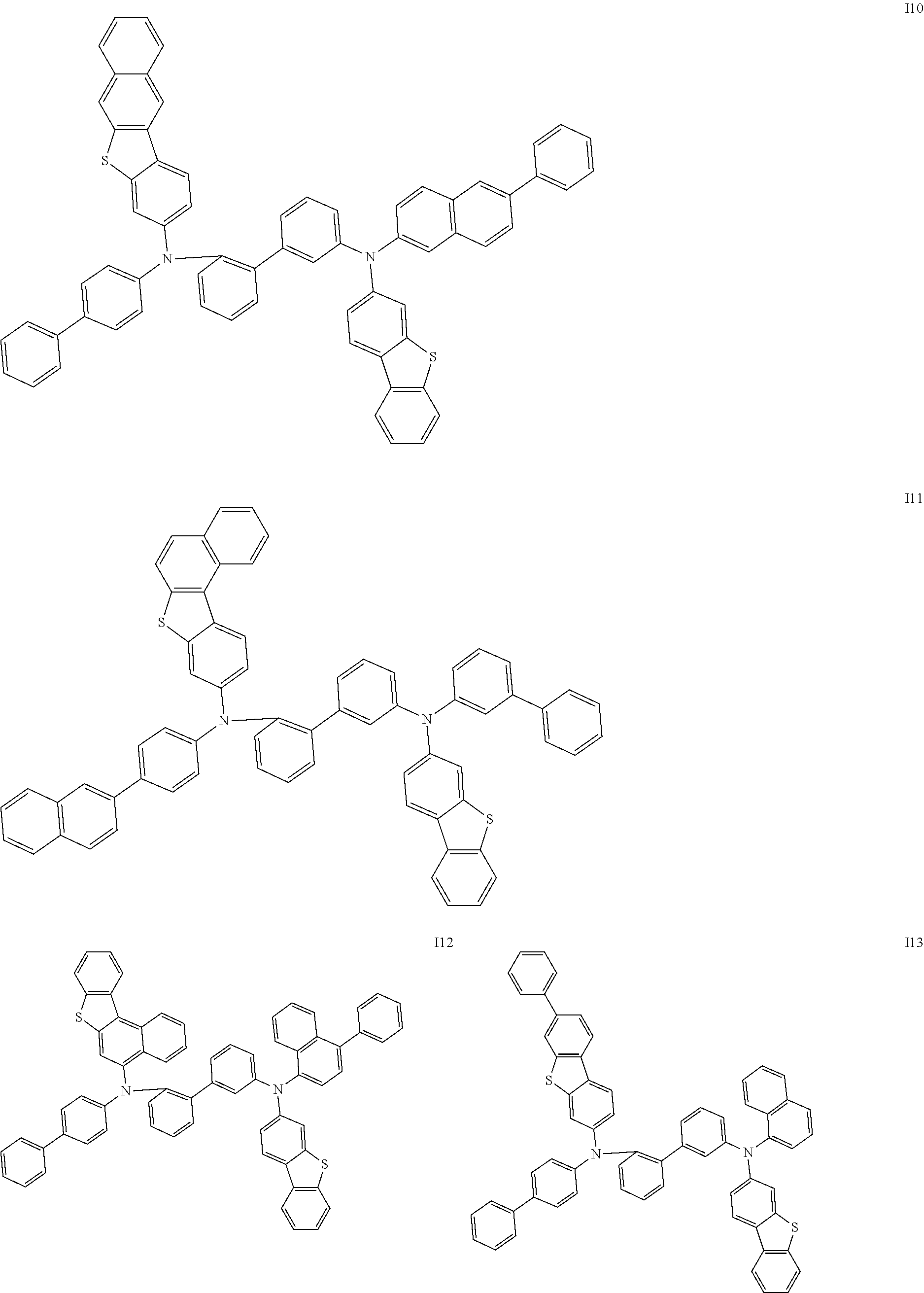 [SMILES: c1ccc(-c2ccc(N(c3ccc4c(c3)sc3cc5ccccc5cc34)c3ccccc3-c3cccc(N(c4ccc5cc(-c6ccccc6)ccc5c4)c4ccc5c(c4)sc4ccccc45)c3)cc2)cc1 ; c1ccc(-c2cccc(N(c3cccc(-c4ccccc4N(c4ccc(-c5ccc6ccccc6c5)cc4)c4ccc5c(c4)sc4ccc6ccccc6c45)c3)c3ccc4c(c3)sc3ccccc34)c2)cc1 ; c1ccc(-c2ccc(N(c3ccccc3-c3cccc(N(c4ccc5c(c4)sc4ccccc45)c4ccc(-c5ccccc5)c5ccccc45)c3)c3cc4sc5ccccc5c4c4ccccc34)cc2)cc1 ; c1ccc(-c2ccc(N(c3ccc4c(c3)sc3cc(-c5ccccc5)ccc34)c3ccccc3-c3cccc(N(c4ccc5c(c4)sc4ccccc45)c4cccc5ccccc45)c3)cc2)cc1]

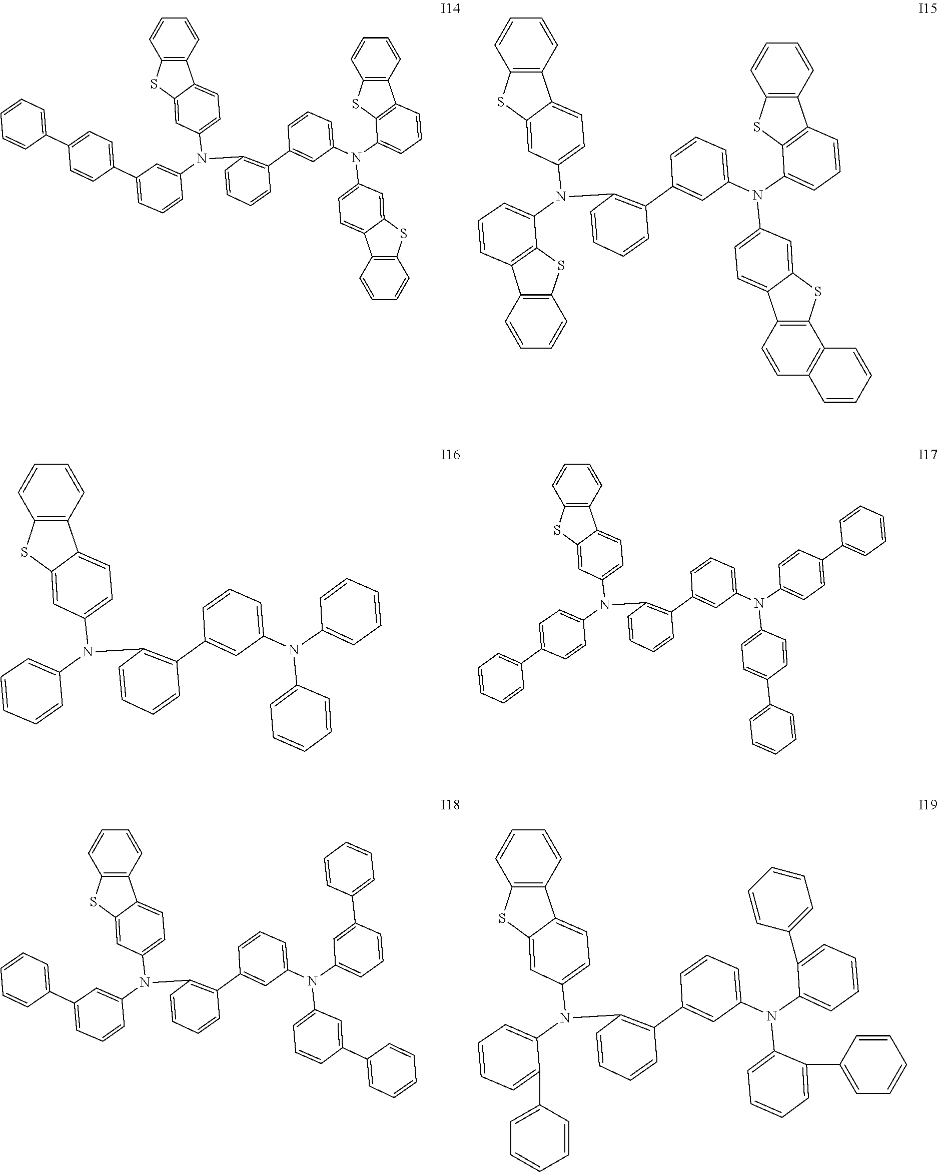

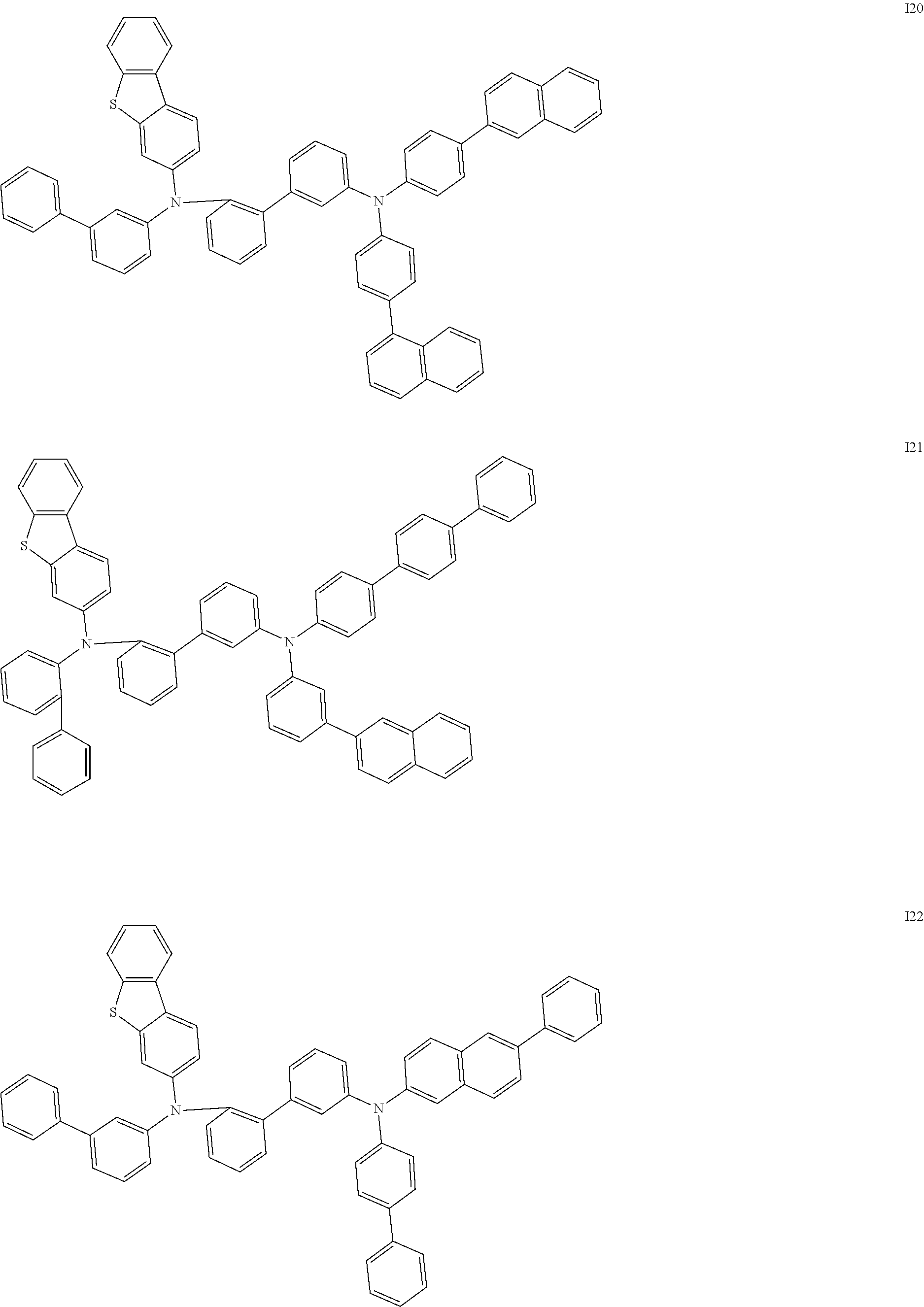 [SMILES: c1ccc(-c2cccc(N(c3ccc4c(c3)sc3ccccc34)c3ccccc3-c3cccc(N(c4ccc(-c5ccc6ccccc6c5)cc4)c4ccc(-c5cccc6ccccc56)cc4)c3)c2)cc1 ; c1ccc(-c2ccc(-c3ccc(N(c4cccc(-c5ccc6ccccc6c5)c4)c4cccc(-c5ccccc5N(c5ccc6c(c5)sc5ccccc56)c5ccccc5-c5ccccc5)c4)cc3)cc2)cc1 ; c1ccc(-c2ccc(N(c3cccc(-c4ccccc4N(c4cccc(-c5ccccc5)c4)c4ccc5c(c4)sc4ccccc45)c3)c3ccc4cc(-c5ccccc5)ccc4c3)cc2)cc1]

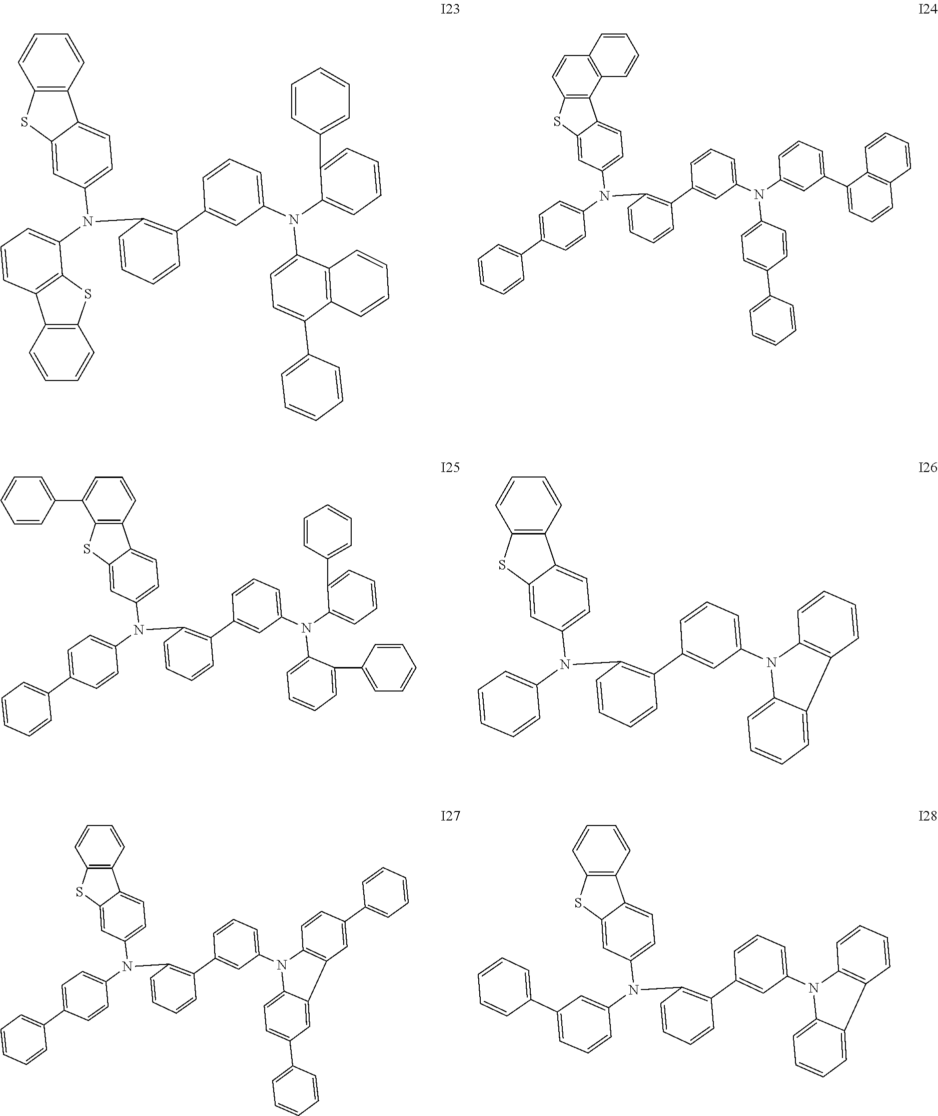

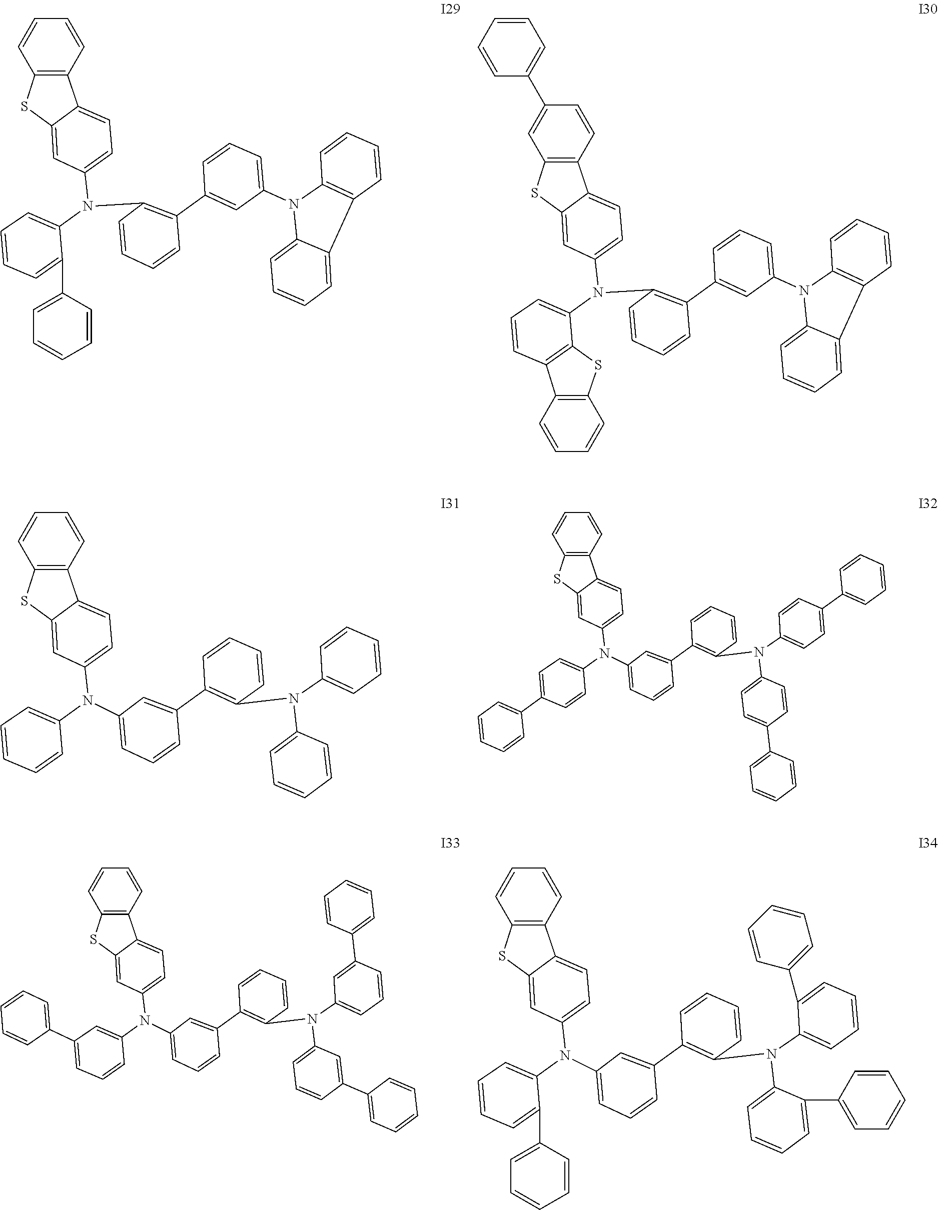

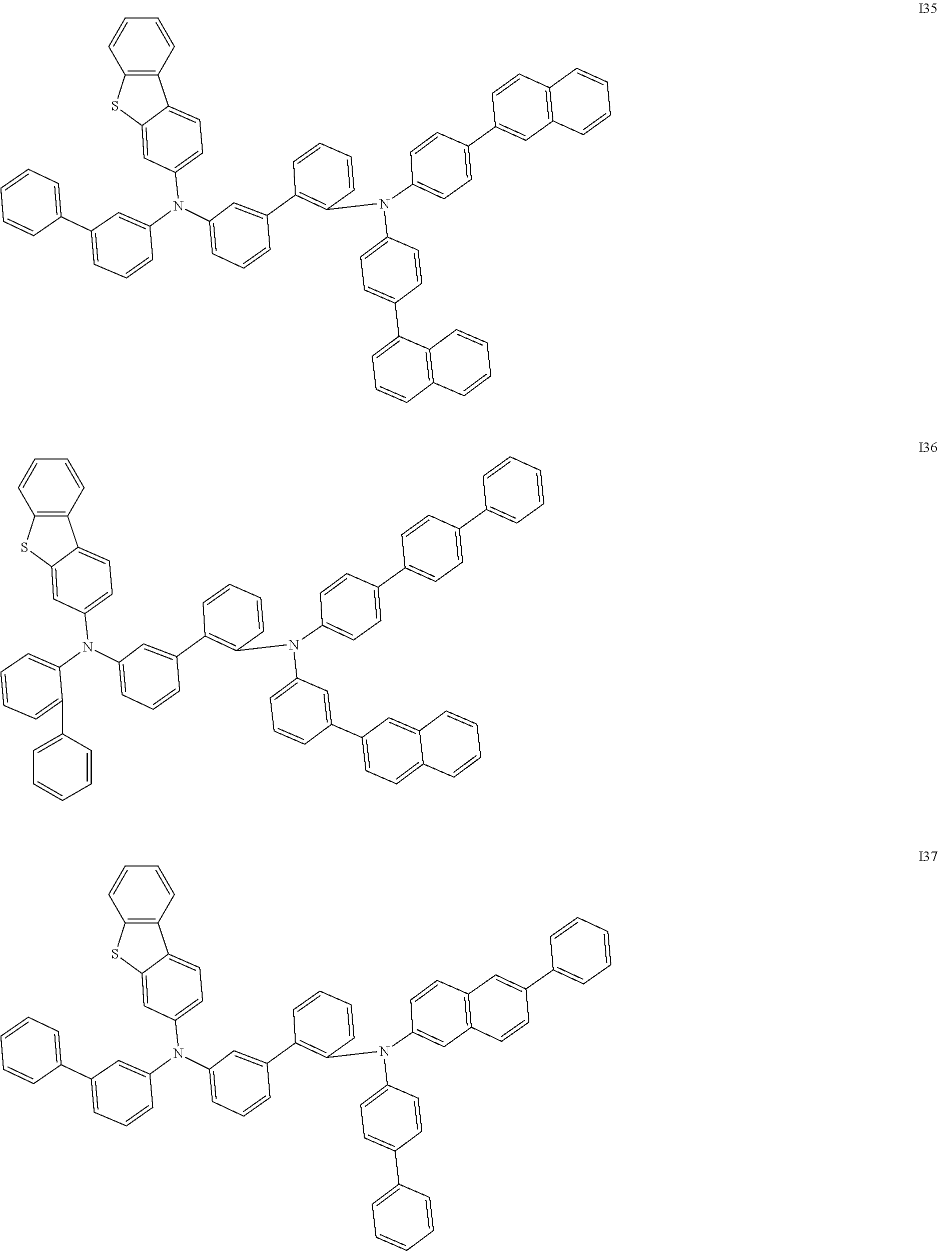 [SMILES: c1ccc(-c2cccc(N(c3cccc(-c4ccccc4N(c4ccc(-c5ccc6ccccc6c5)cc4)c4ccc(-c5cccc6ccccc56)cc4)c3)c3ccc4c(c3)sc3ccccc34)c2)cc1 ; c1ccc(-c2ccc(-c3ccc(N(c4cccc(-c5ccc6ccccc6c5)c4)c4ccccc4-c4cccc(N(c5ccc6c(c5)sc5ccccc56)c5ccccc5-c5ccccc5)c4)cc3)cc2)cc1 ; c1ccc(-c2ccc(N(c3ccc4cc(-c5ccccc5)ccc4c3)c3ccccc3-c3cccc(N(c4cccc(-c5ccccc5)c4)c4ccc5c(c4)sc4ccccc45)c3)cc2)cc1]

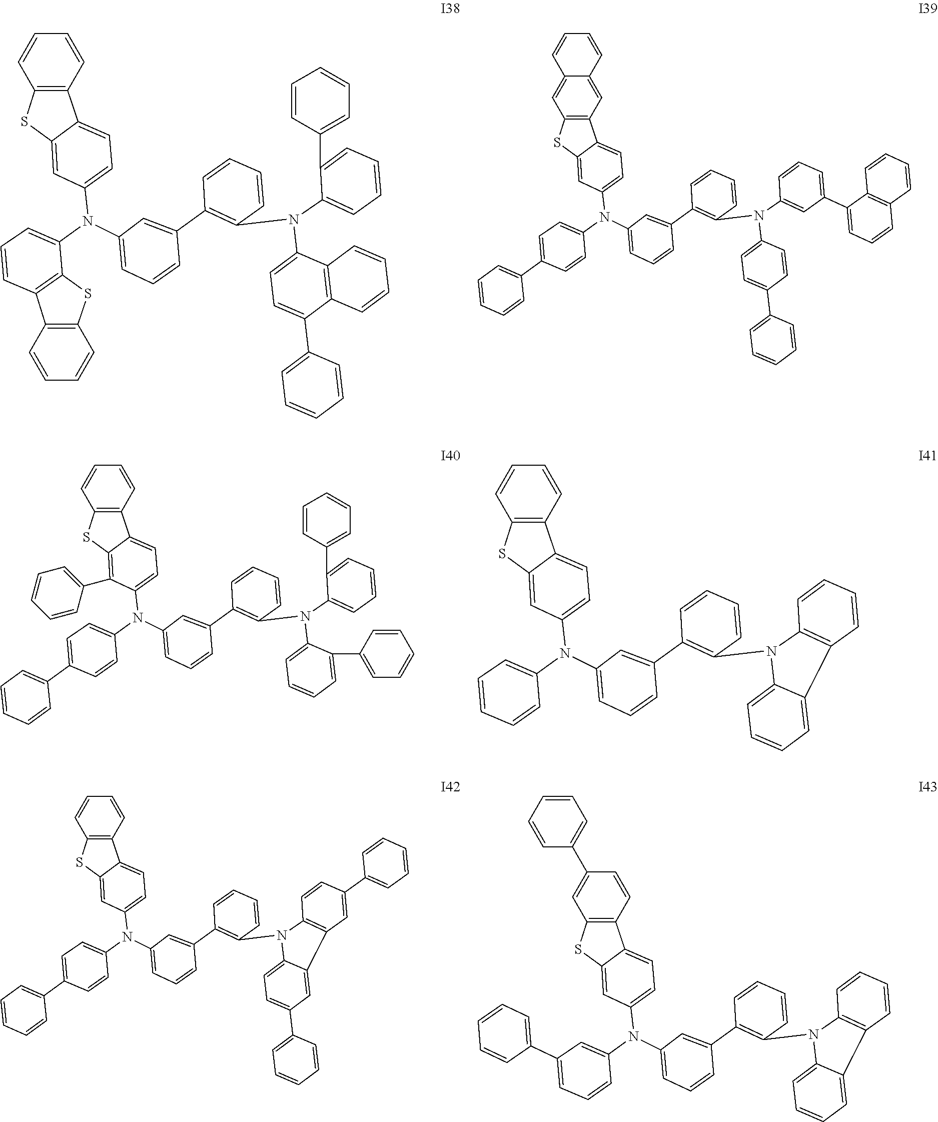

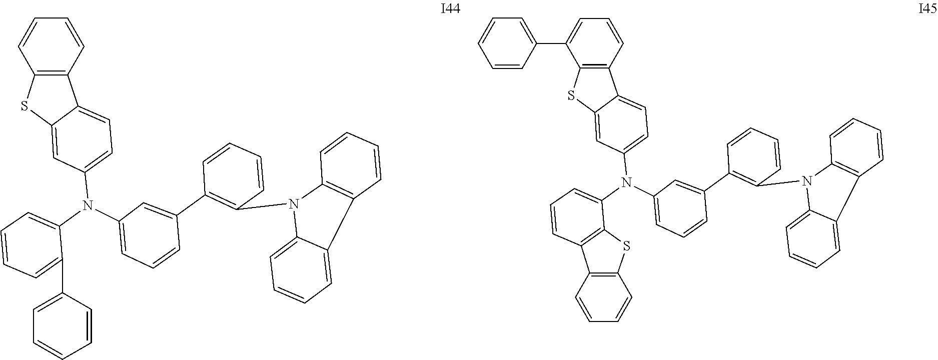 [SMILES: c1ccc(-c2ccccc2N(c2cccc(-c3ccccc3-n3c4ccccc4c4ccccc43)c2)c2ccc3c(c2)sc2ccccc23)cc1 ; c1ccc(-c2cccc3c2sc2cc(N(c4cccc(-c5ccccc5-n5c6ccccc6c6ccccc65)c4)c4cccc5c4sc4ccccc45)ccc23)cc1]

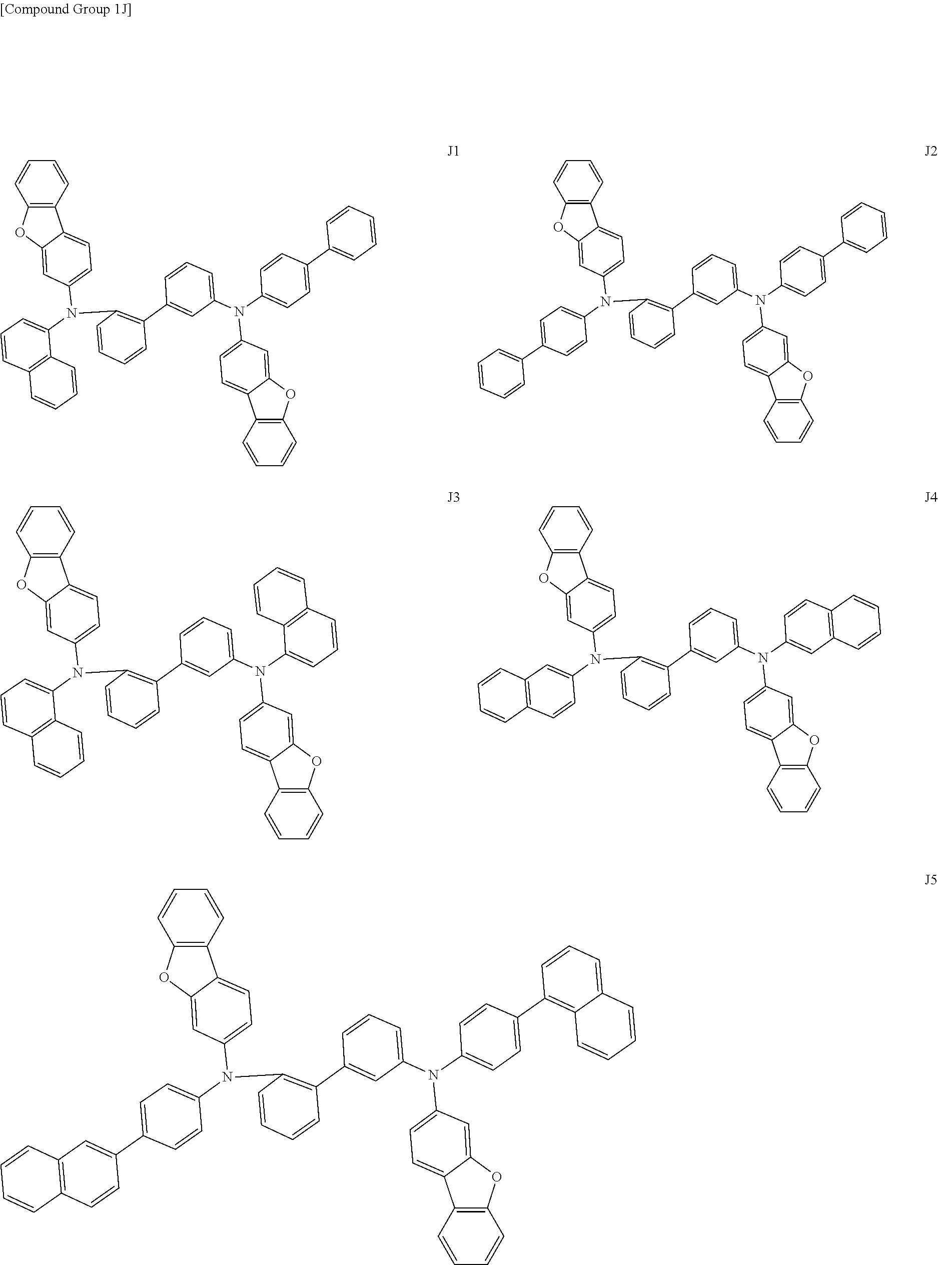

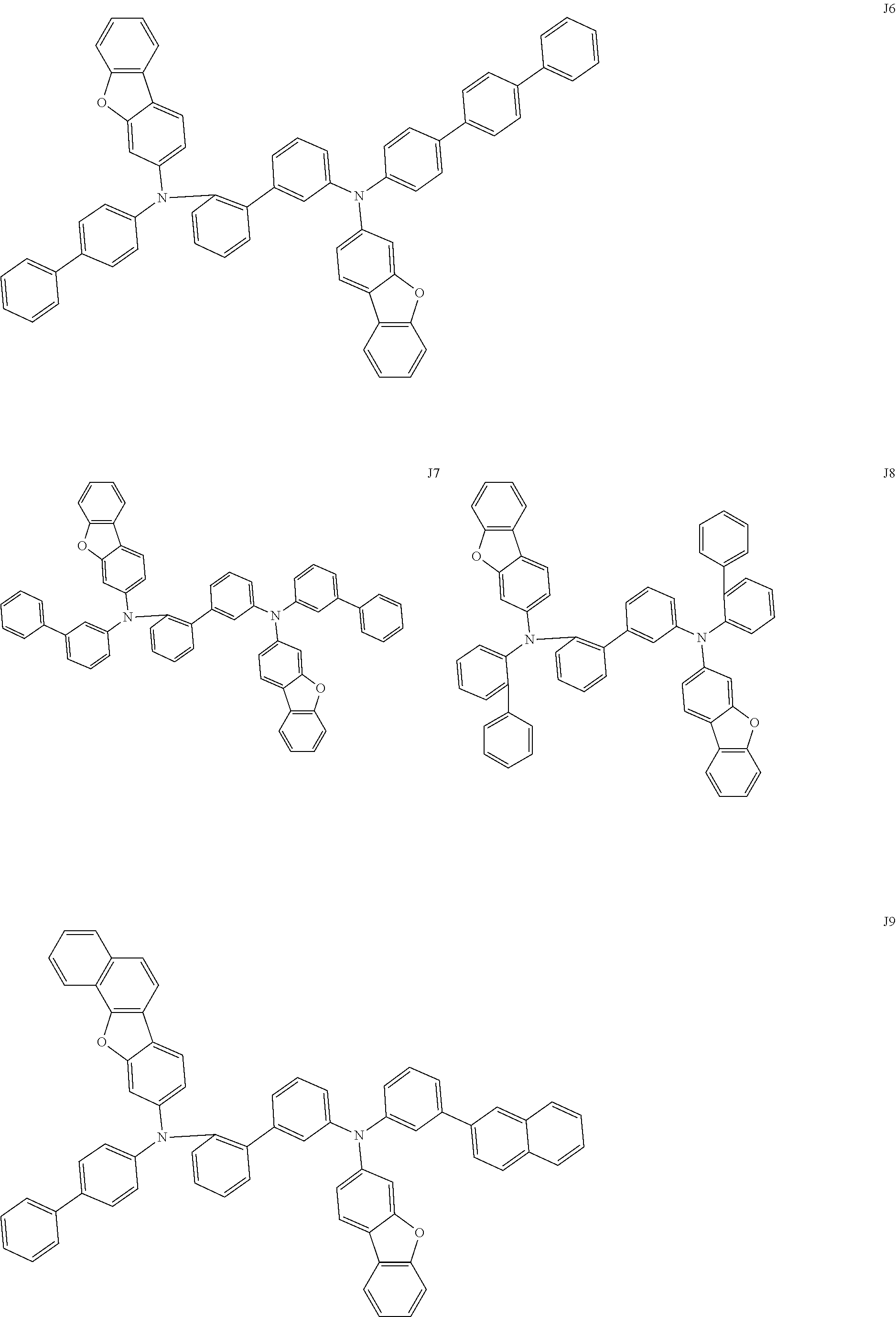 [SMILES: c1ccc(-c2ccc(-c3ccc(N(c4cccc(-c5ccccc5N(c5ccc(-c6ccccc6)cc5)c5ccc6c(c5)oc5ccccc56)c4)c4ccc5c(c4)oc4ccccc45)cc3)cc2)cc1 ; c1ccc(-c2cccc(N(c3cccc(-c4ccccc4N(c4cccc(-c5ccccc5)c4)c4ccc5c(c4)oc4ccccc45)c3)c3ccc4c(c3)oc3ccccc34)c2)cc1 ; c1ccc(-c2ccccc2N(c2cccc(-c3ccccc3N(c3ccc4c(c3)oc3ccccc34)c3ccccc3-c3ccccc3)c2)c2ccc3c(c2)oc2ccccc23)cc1 ; c1ccc(-c2ccc(N(c3ccc4c(c3)oc3c5ccccc5ccc43)c3ccccc3-c3cccc(N(c4cccc(-c5ccc6ccccc6c5)c4)c4ccc5c(c4)oc4ccccc45)c3)cc2)cc1]

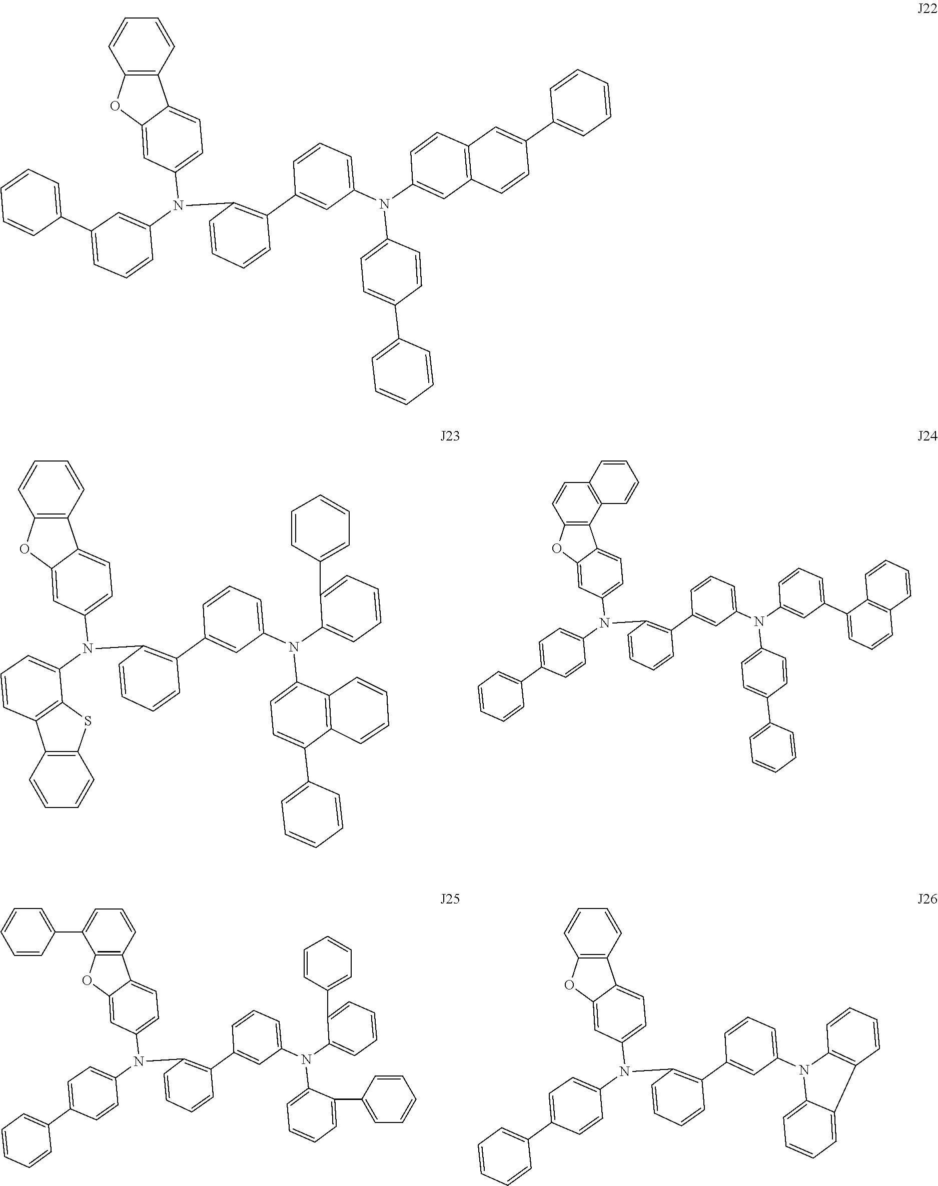

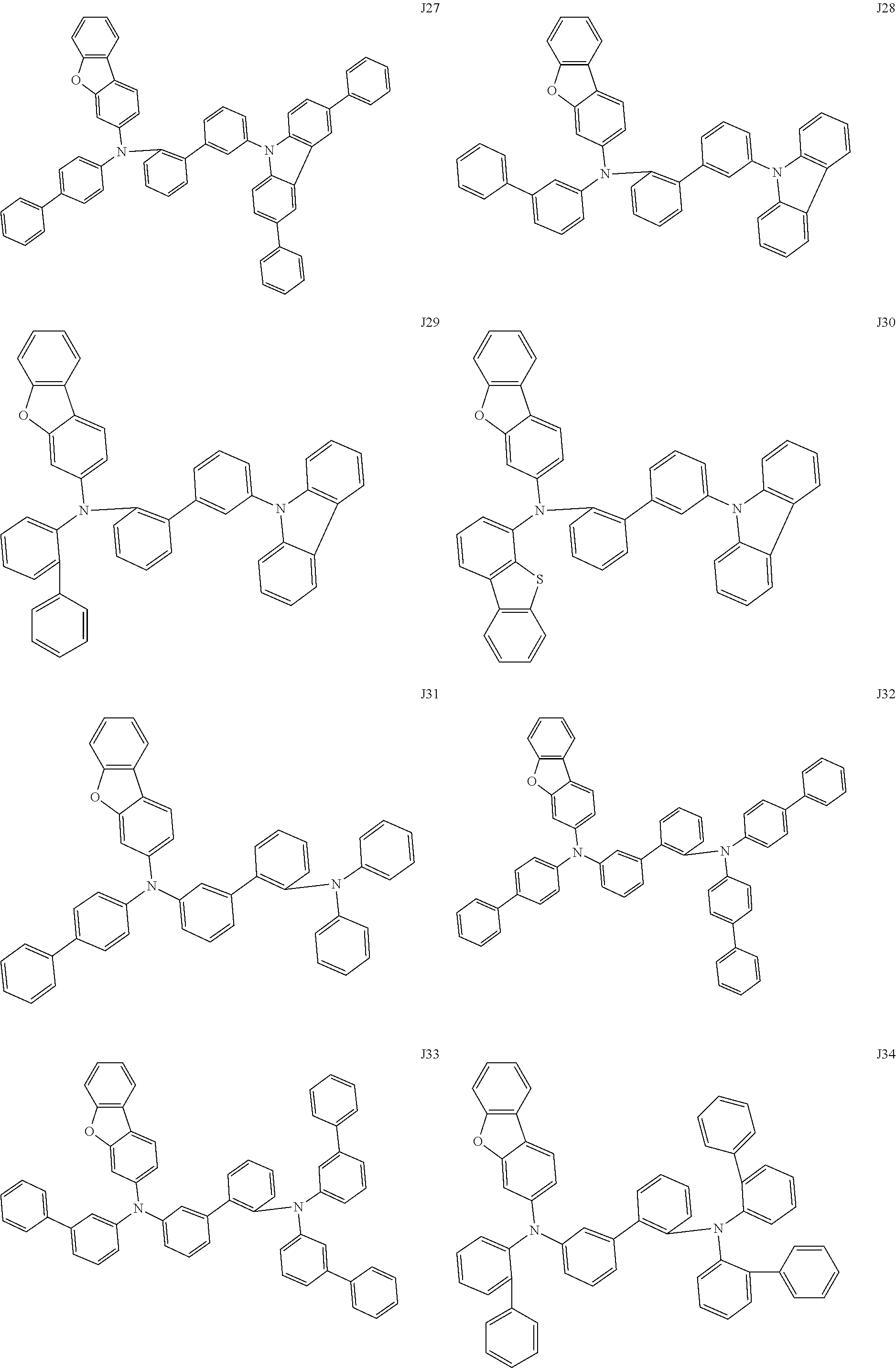

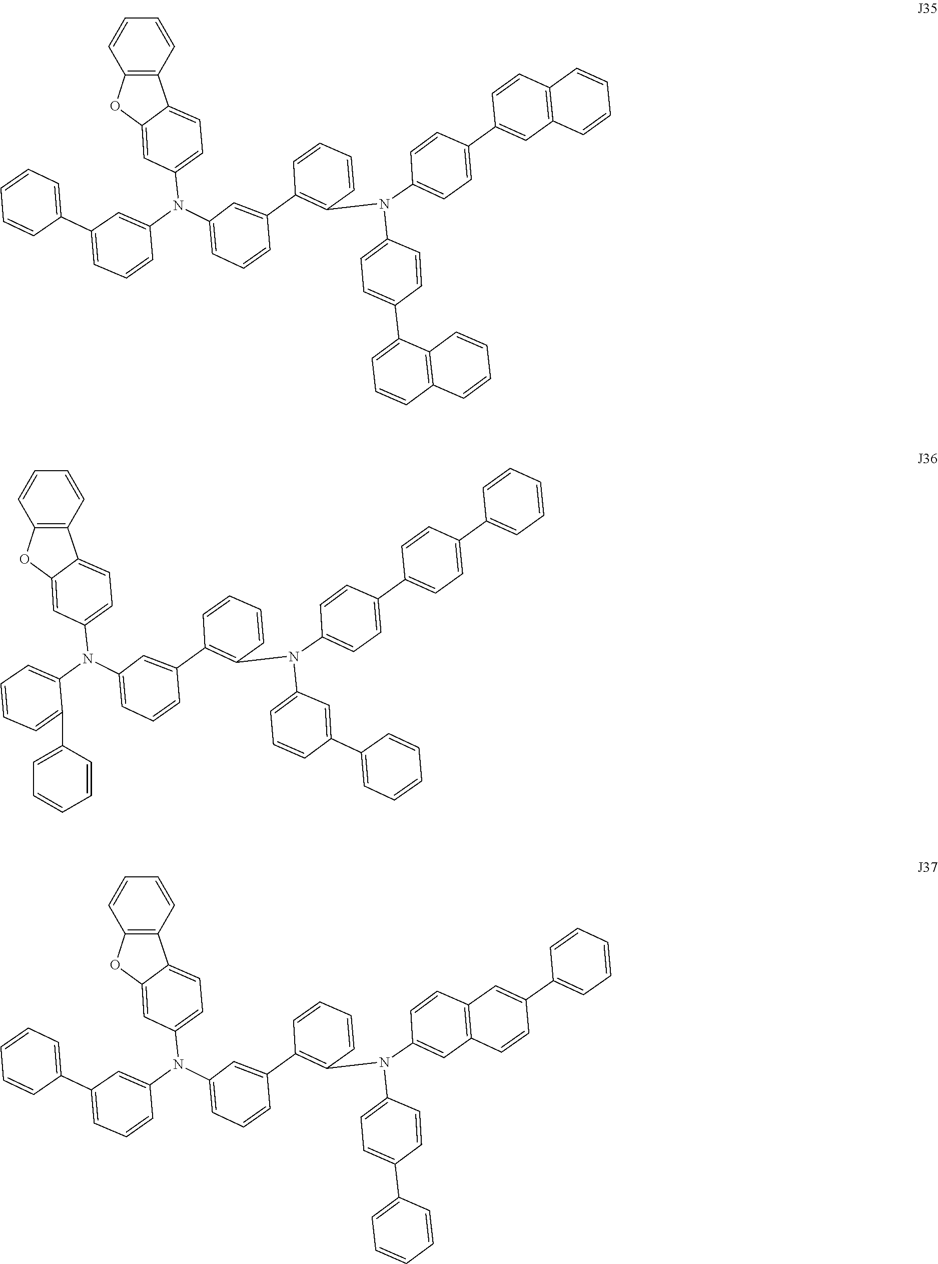 [SMILES: c1ccc(-c2cccc(N(c3cccc(-c4ccccc4N(c4ccc(-c5ccc6ccccc6c5)cc4)c4ccc(-c5cccc6ccccc56)cc4)c3)c3ccc4c(c3)oc3ccccc34)c2)cc1 ; c1ccc(-c2ccc(-c3ccc(N(c4cccc(-c5ccccc5)c4)c4ccccc4-c4cccc(N(c5ccc6c(c5)oc5ccccc56)c5ccccc5-c5ccccc5)c4)cc3)cc2)cc1 ; c1ccc(-c2ccc(N(c3ccc4cc(-c5ccccc5)ccc4c3)c3ccccc3-c3cccc(N(c4cccc(-c5ccccc5)c4)c4ccc5c(c4)oc4ccccc45)c3)cc2)cc1]

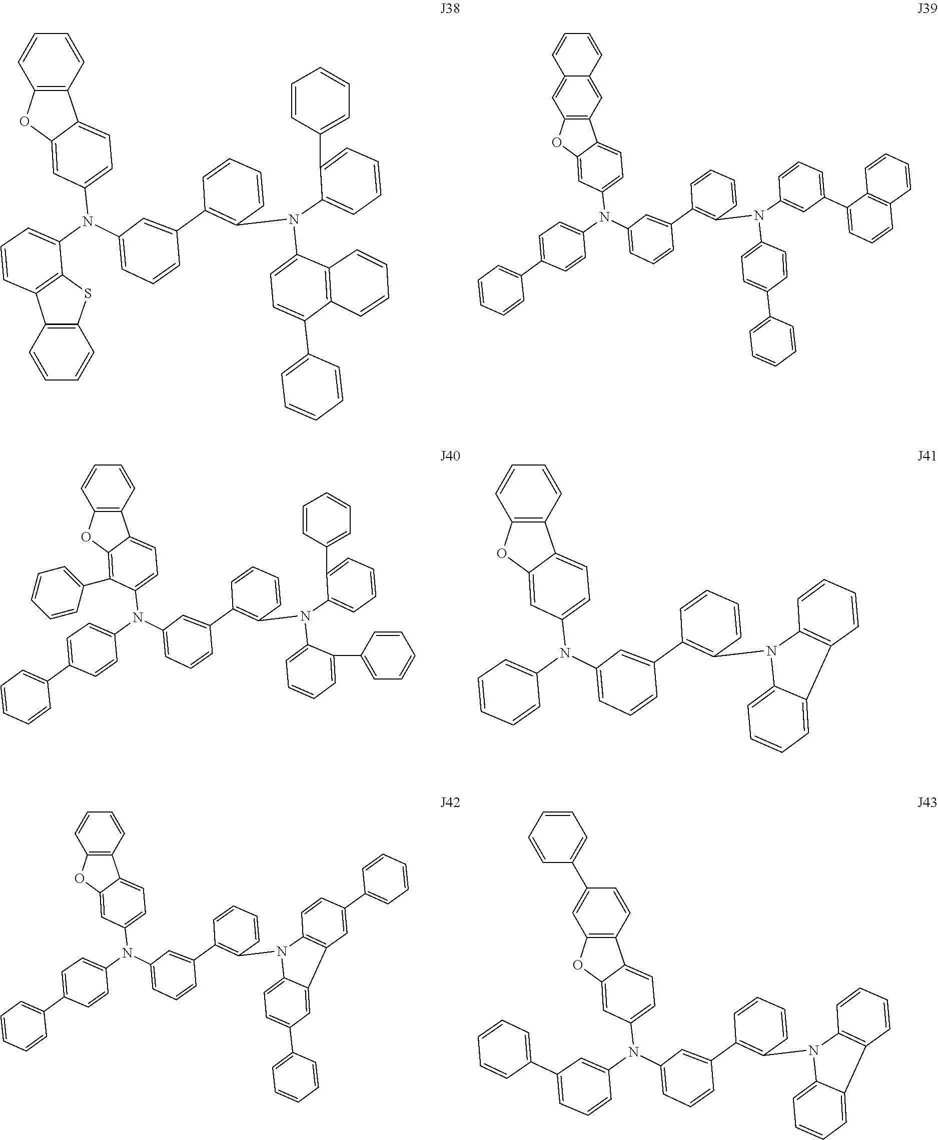

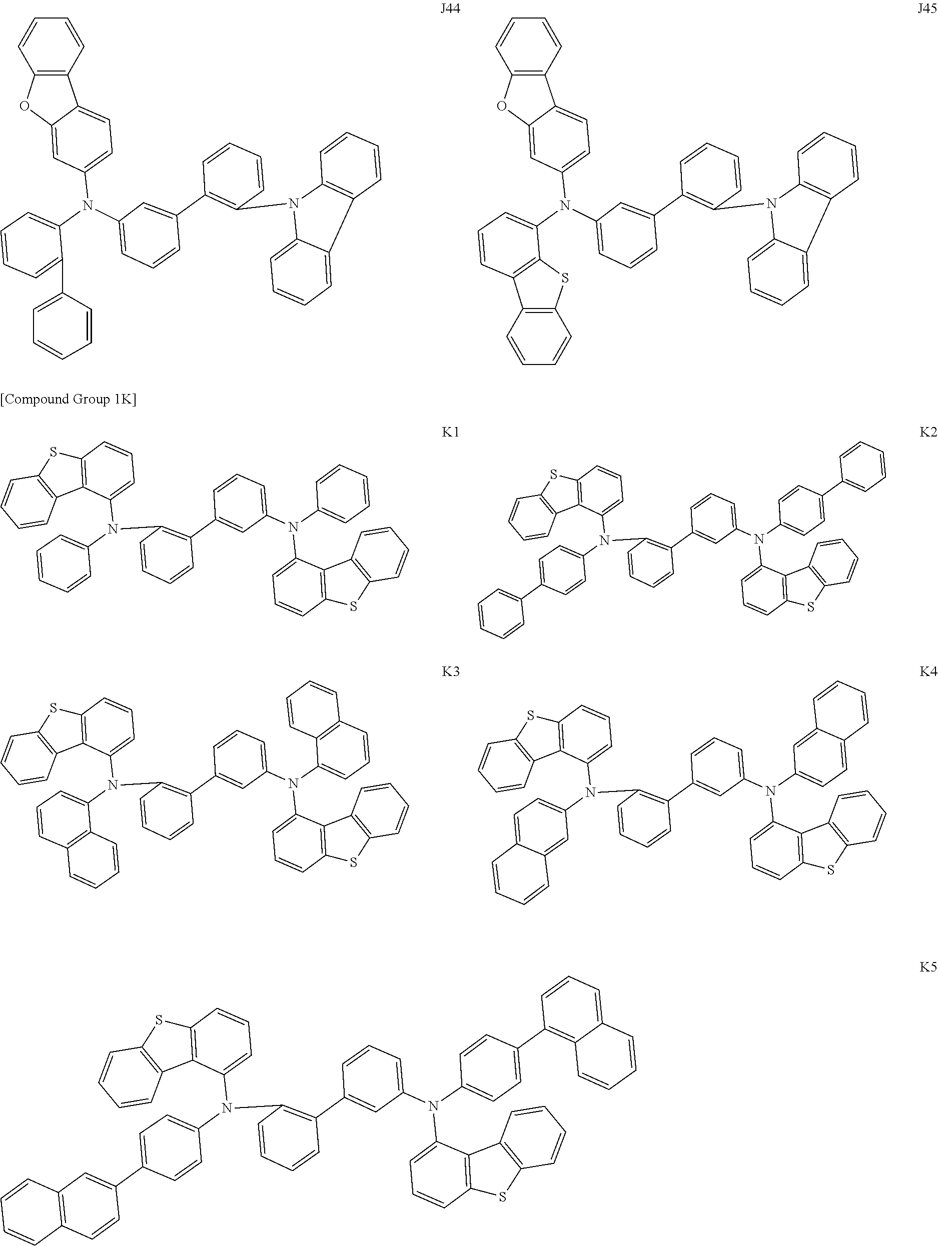

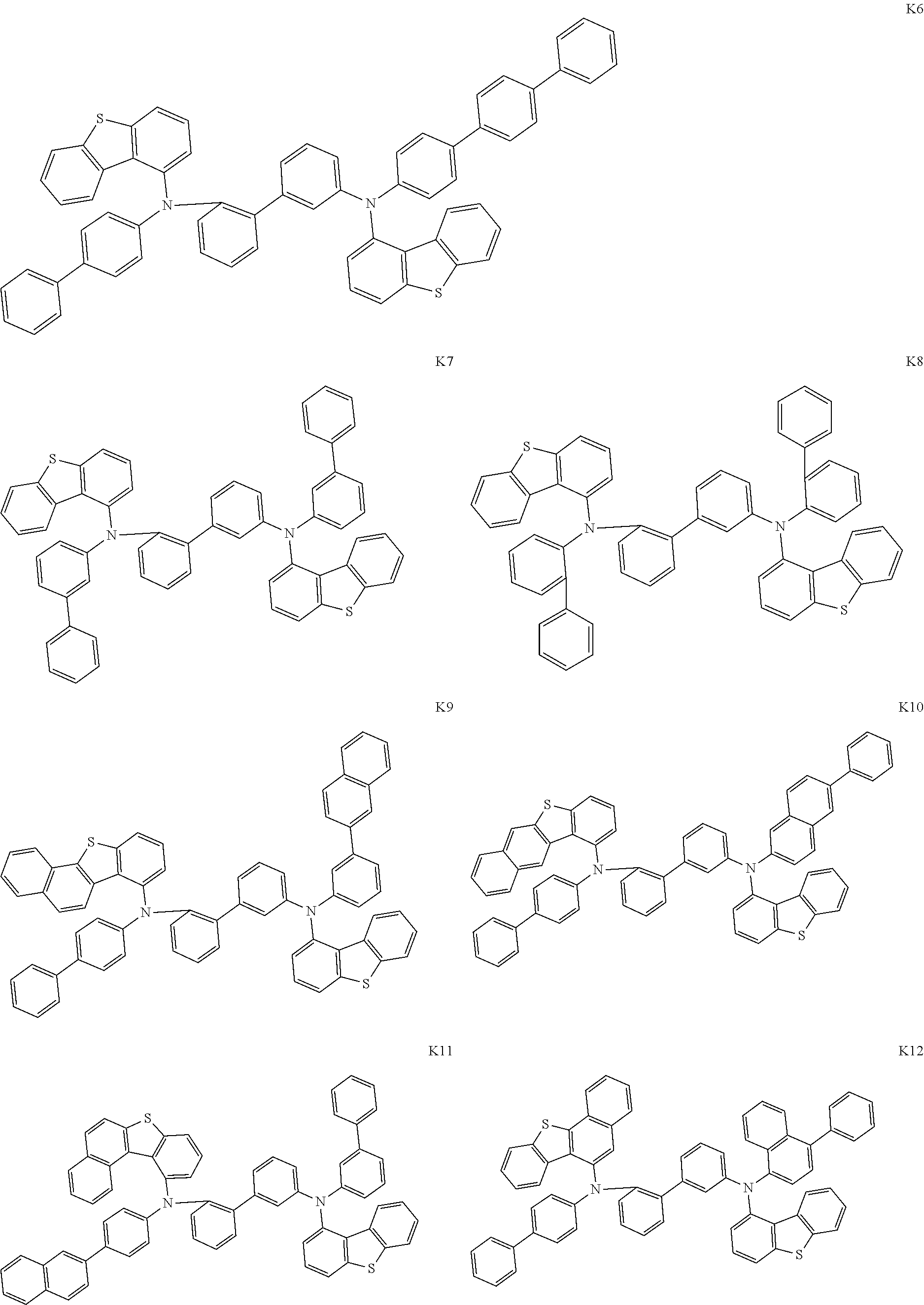

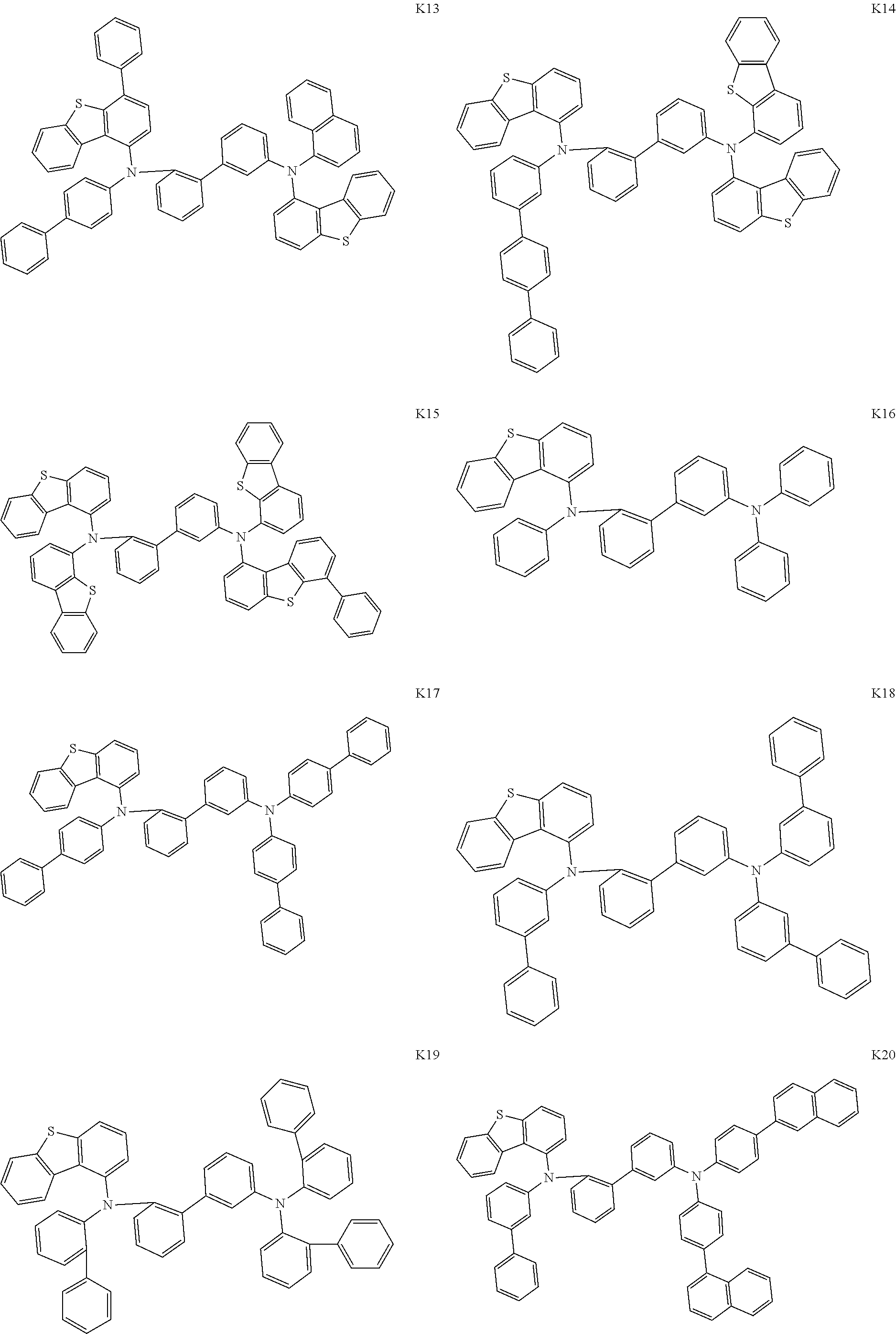

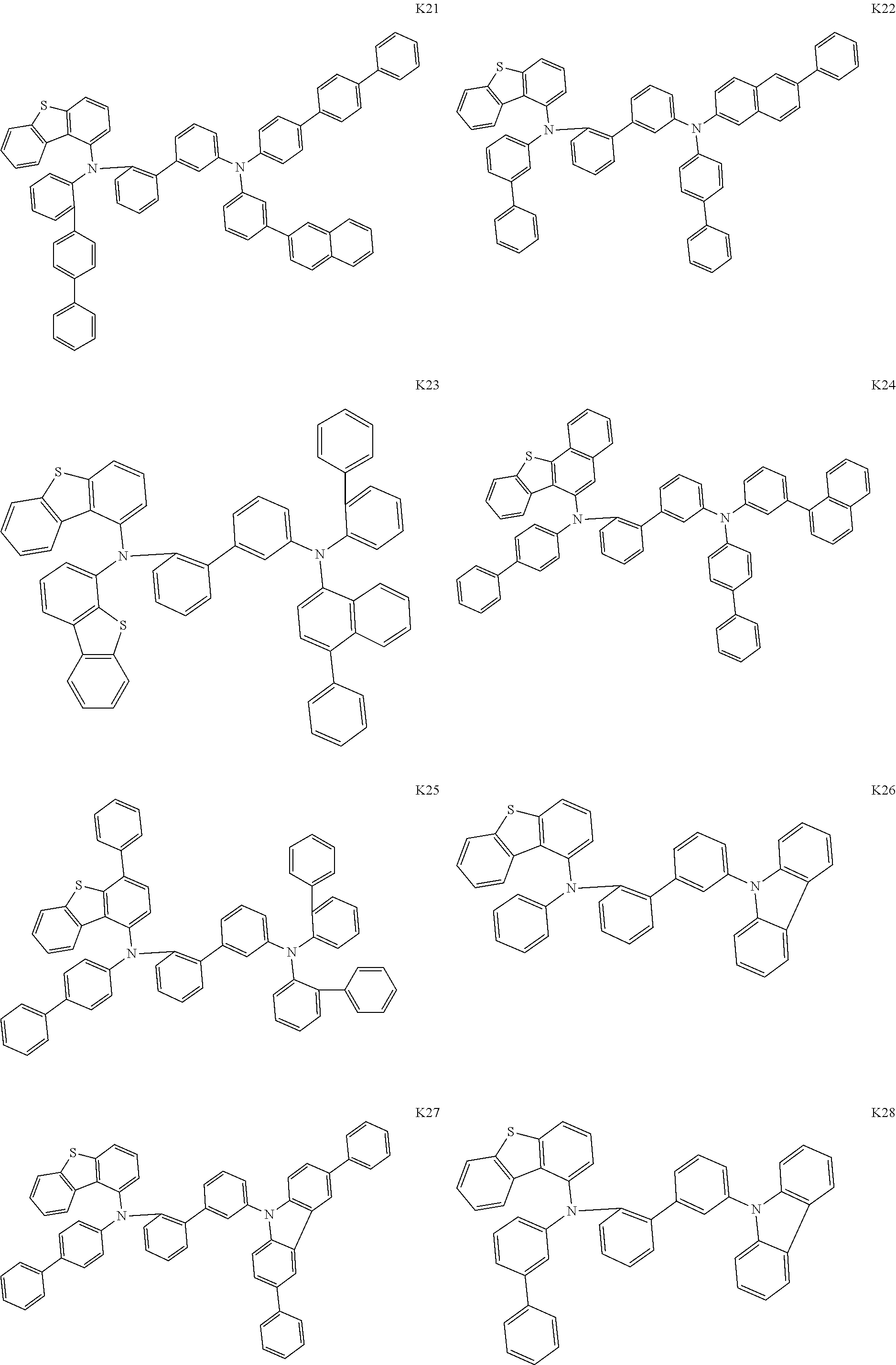

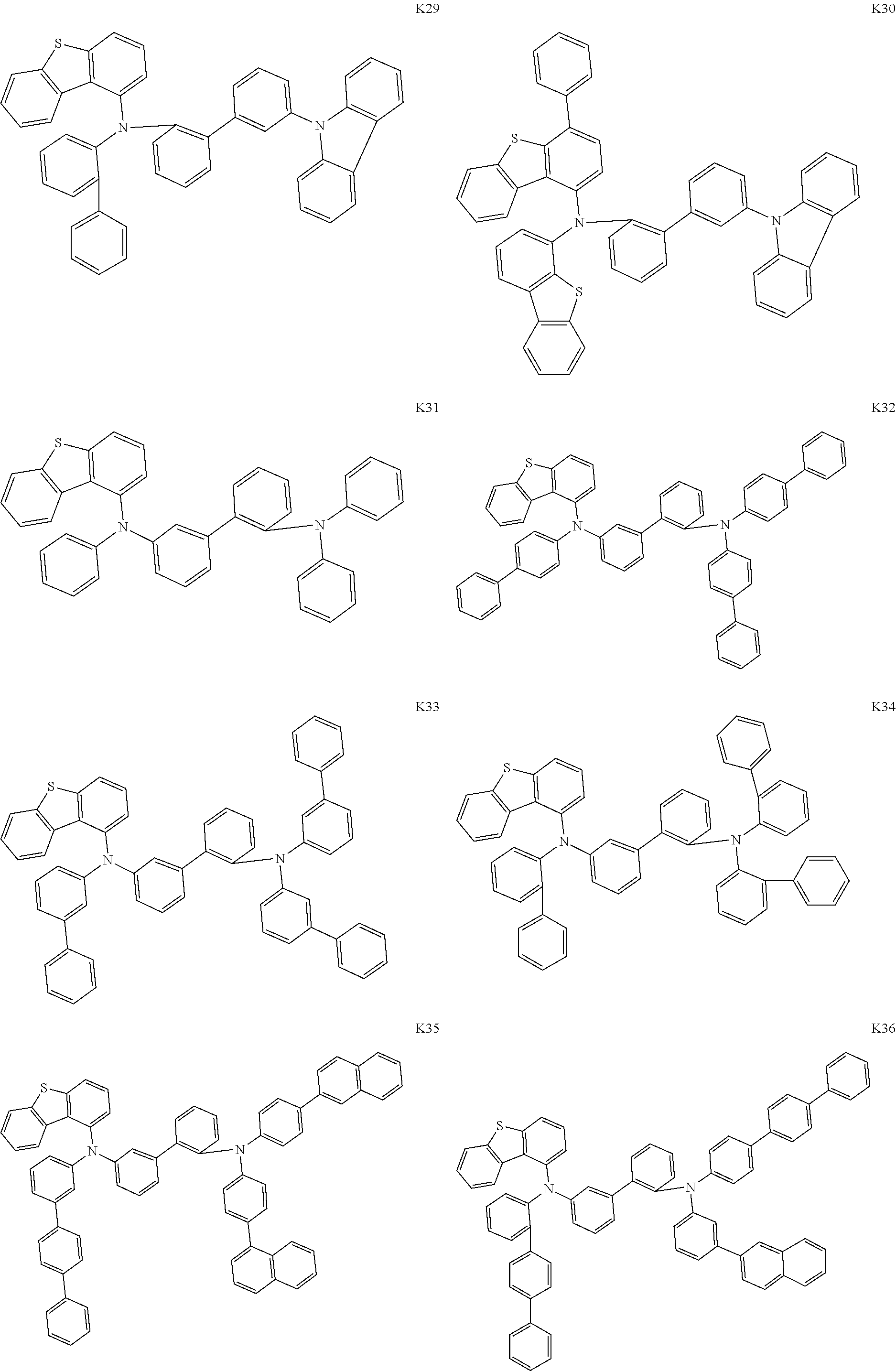

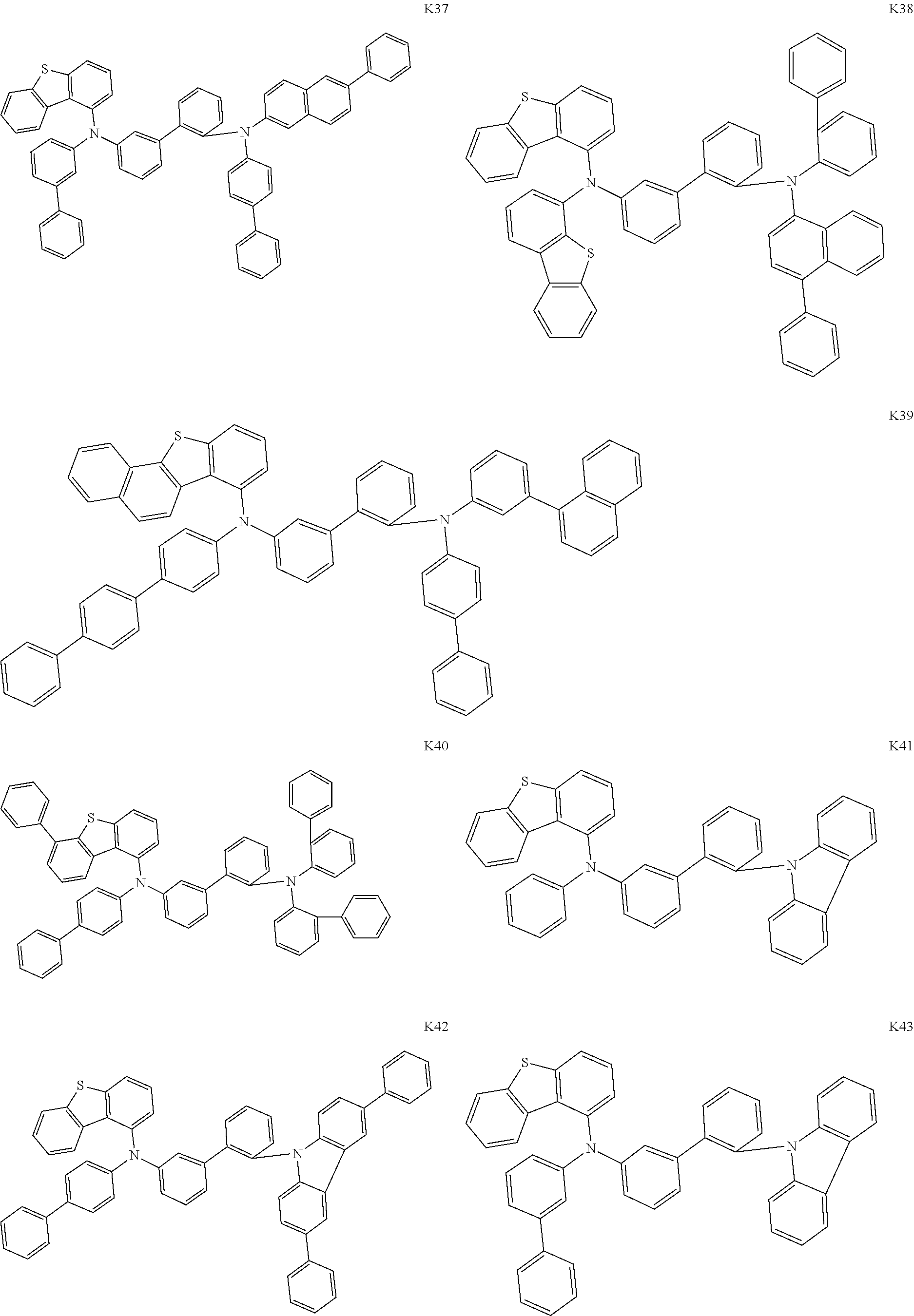

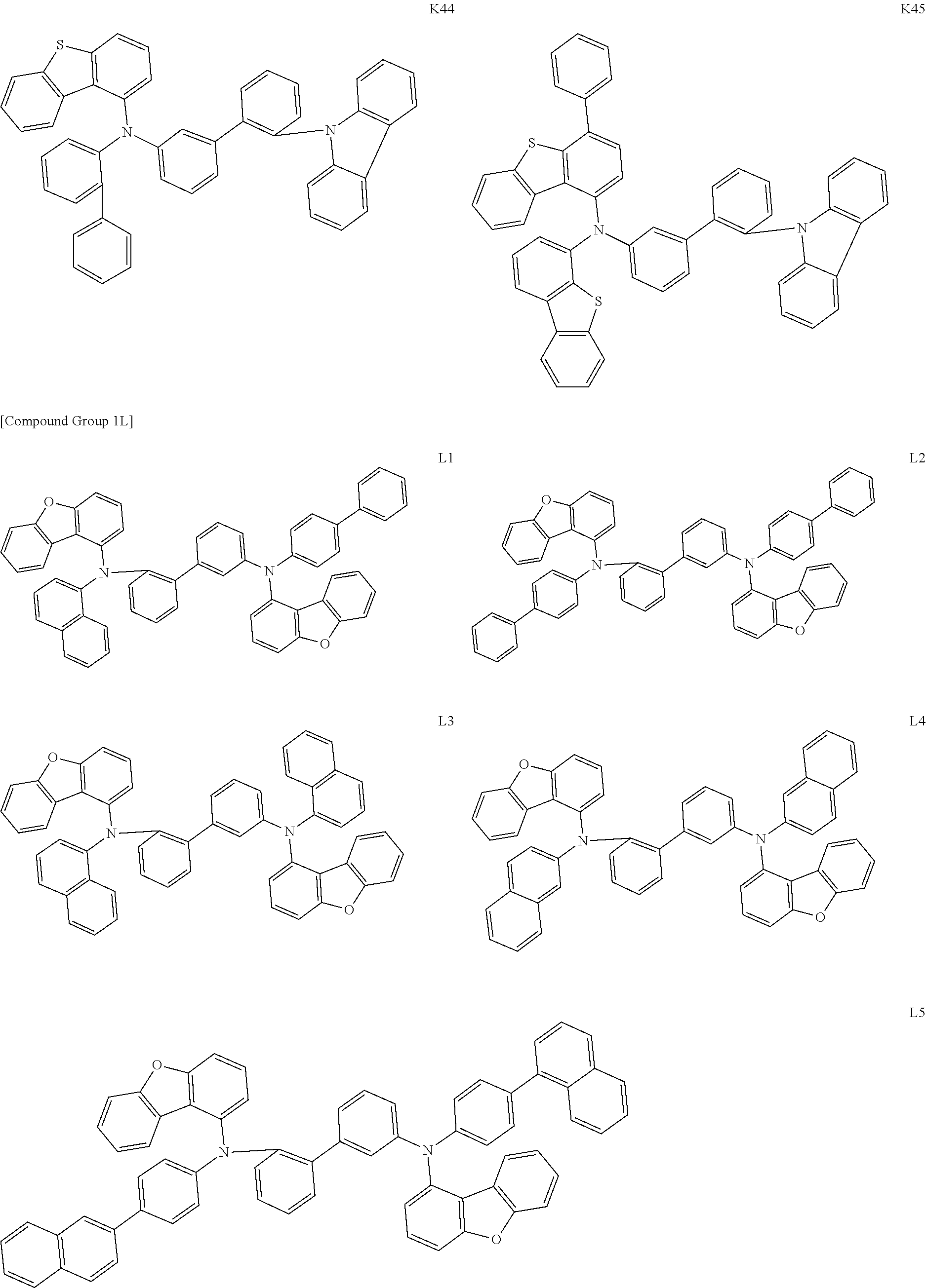

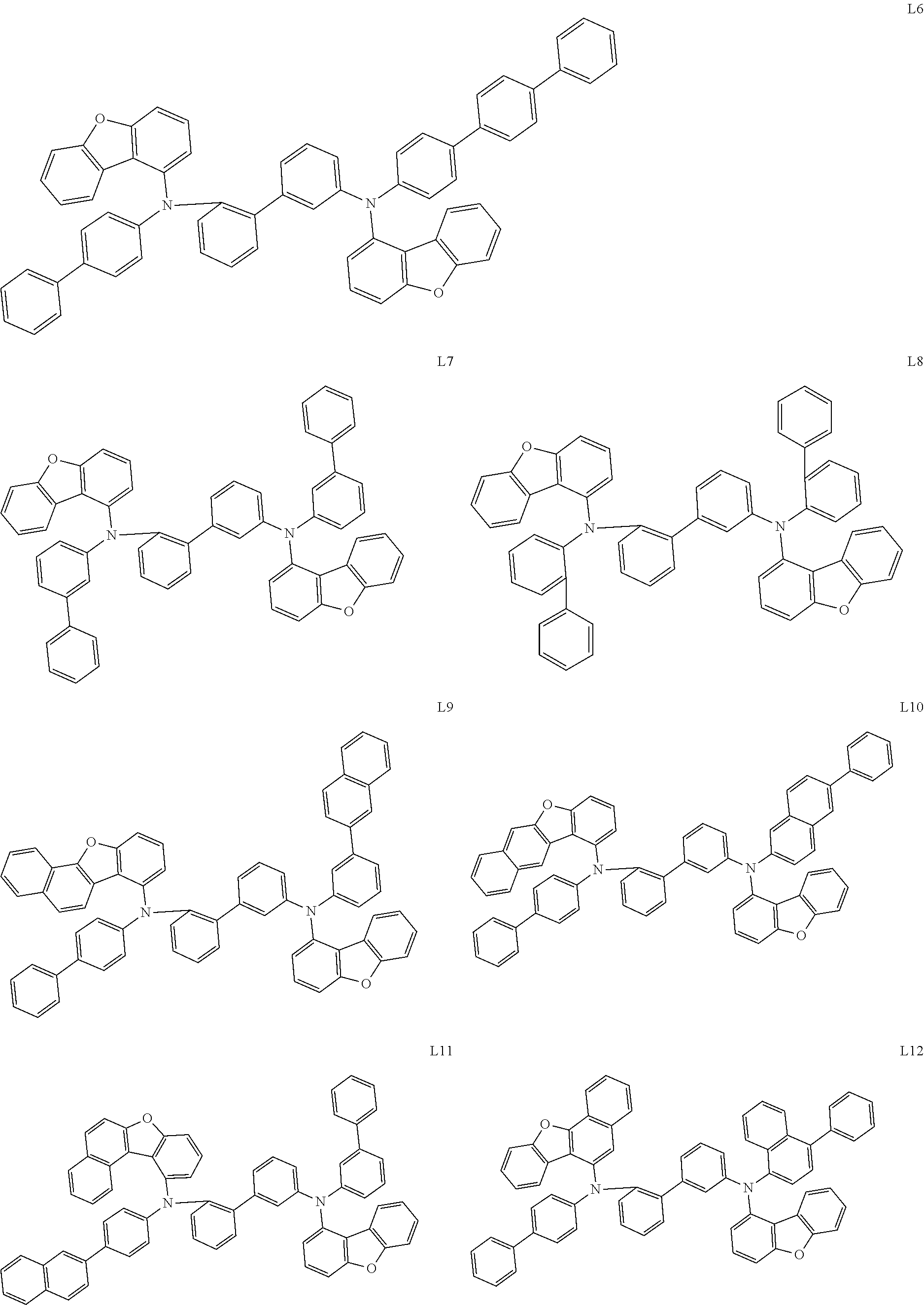

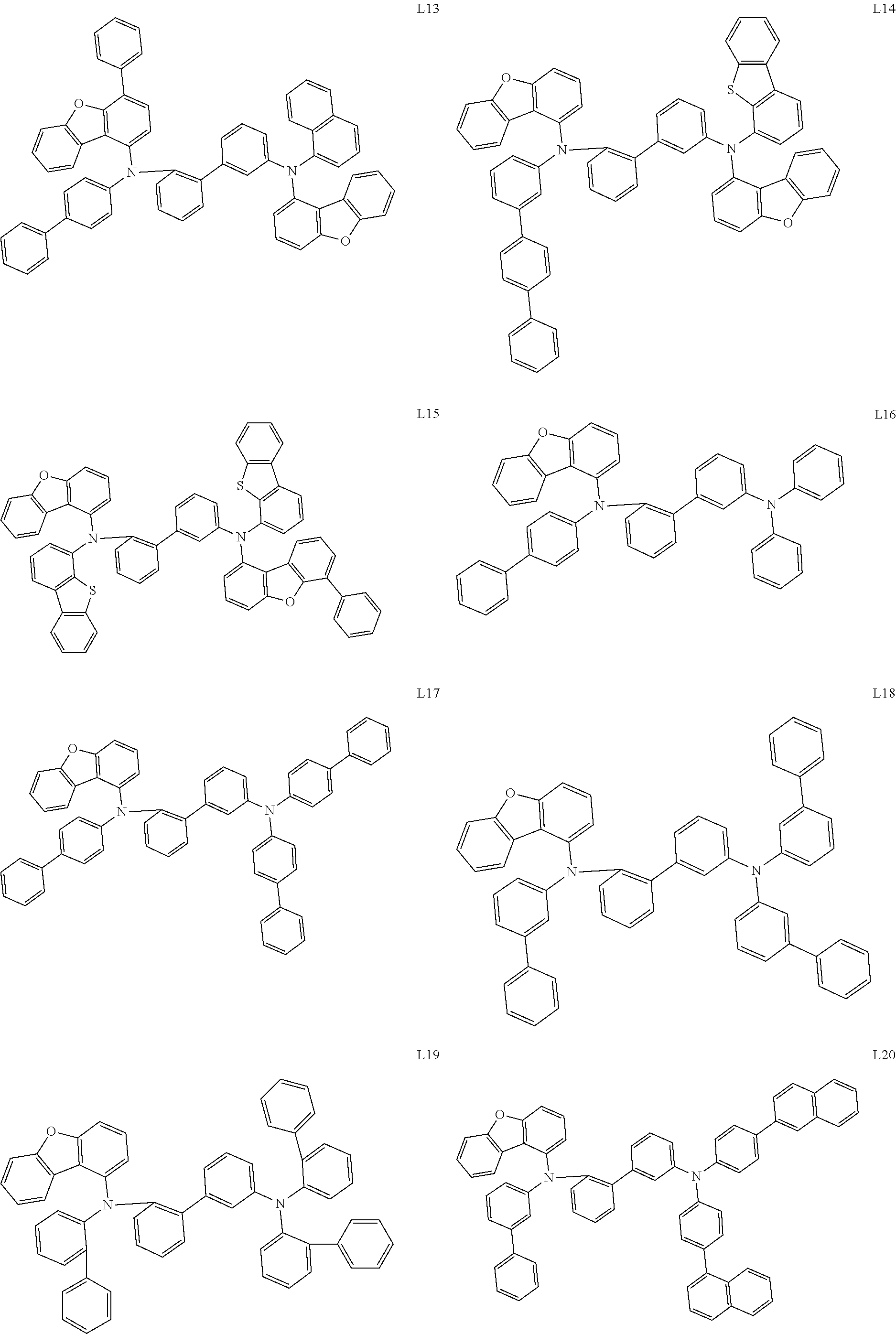

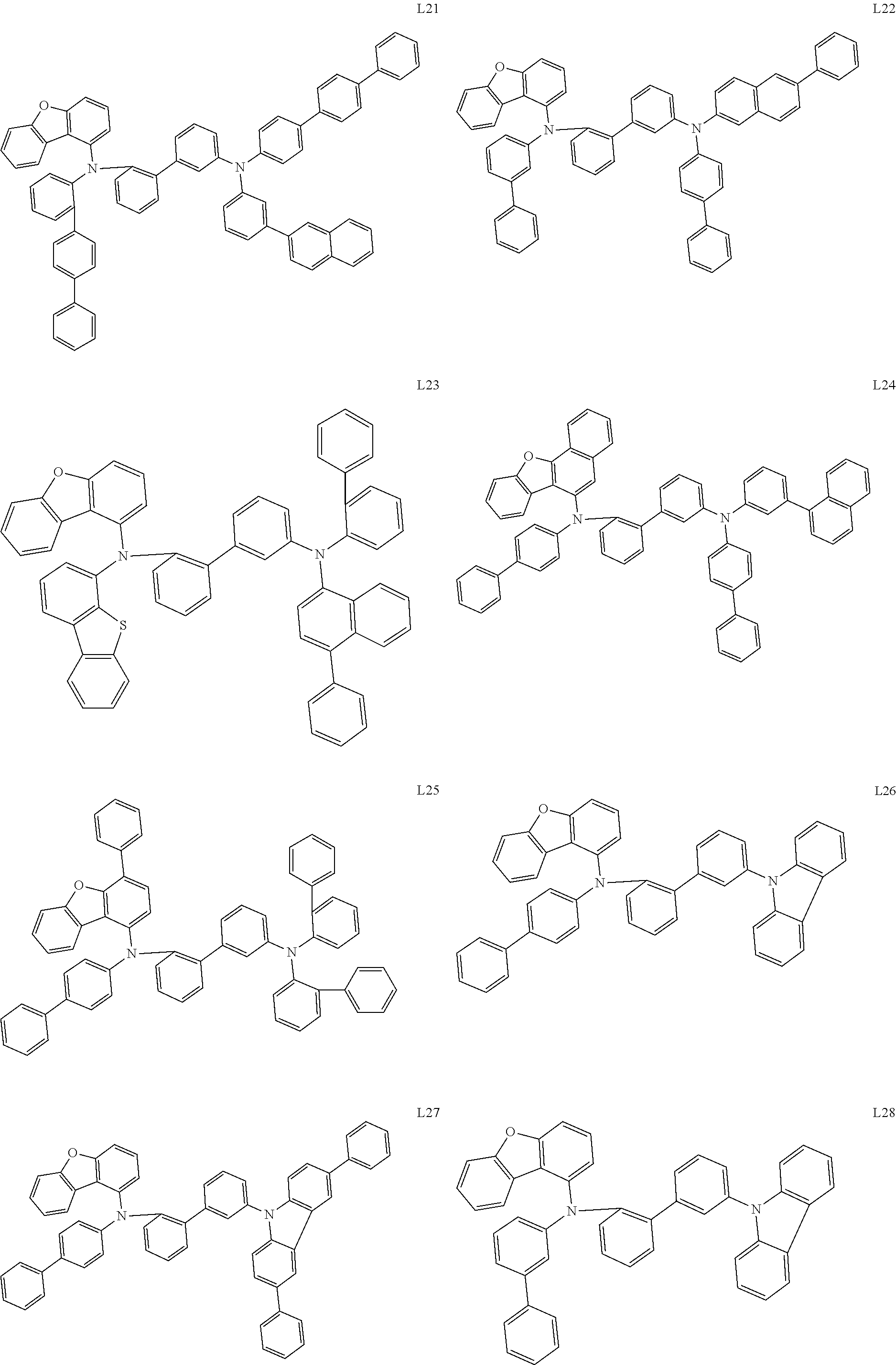

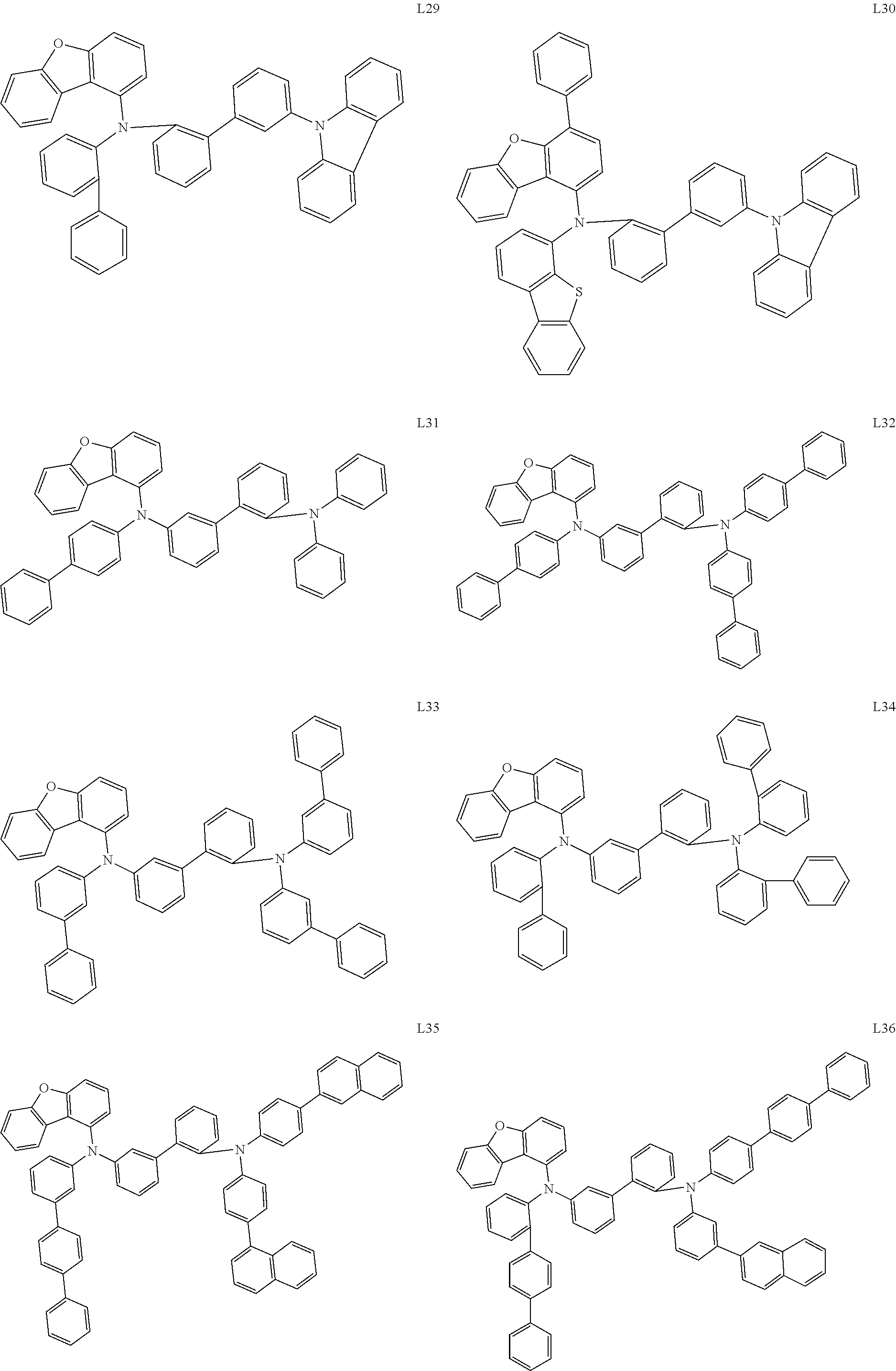

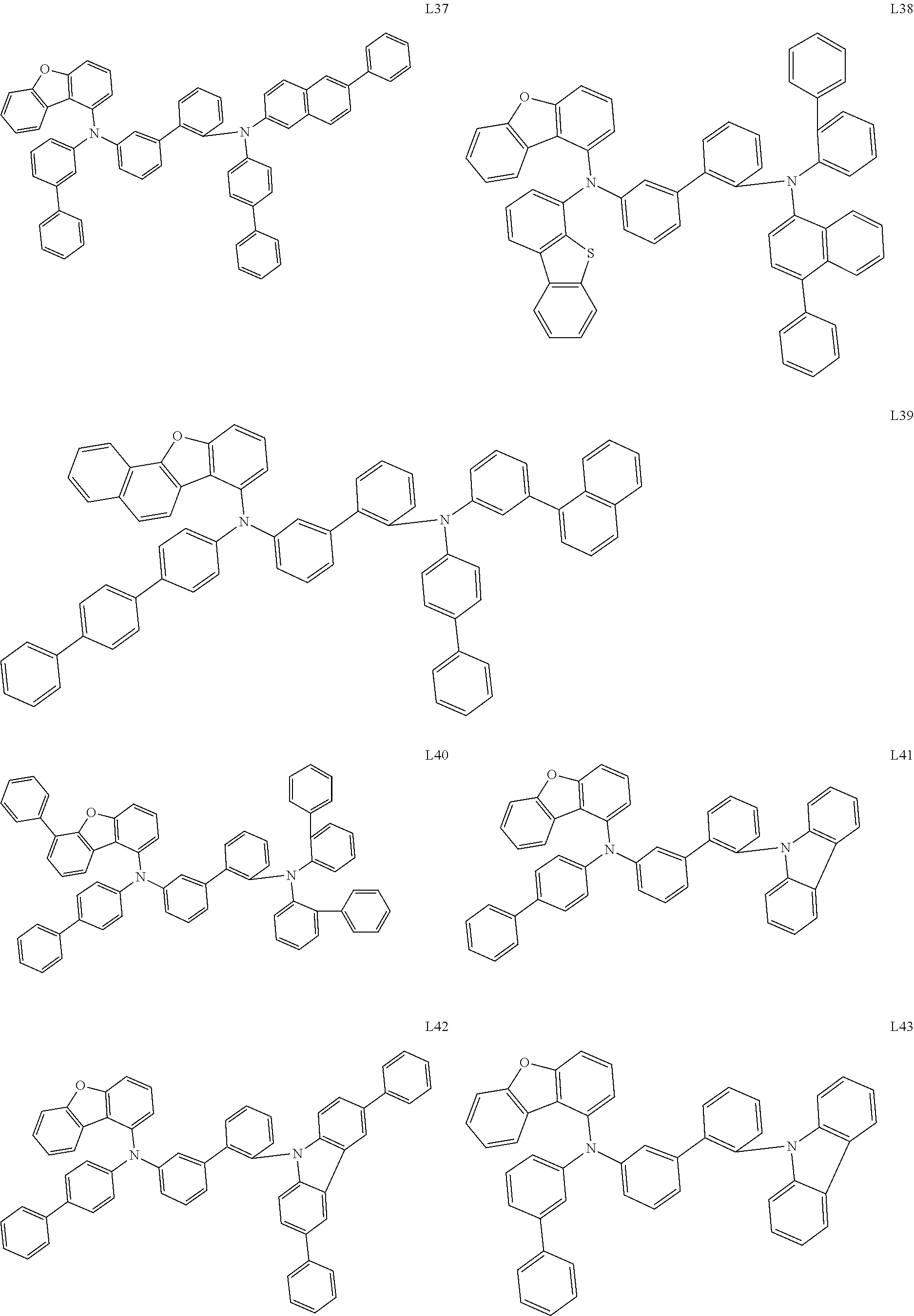

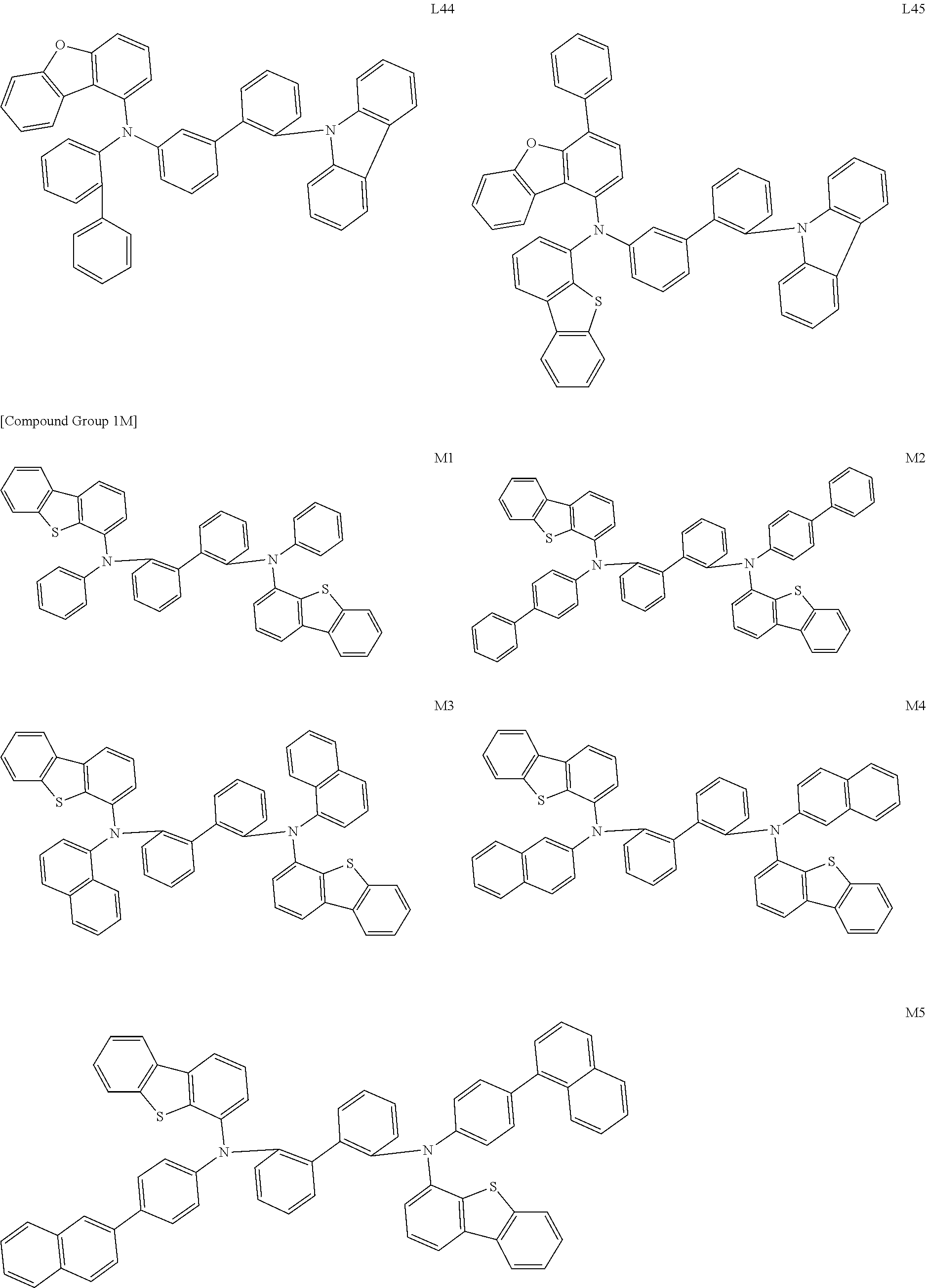

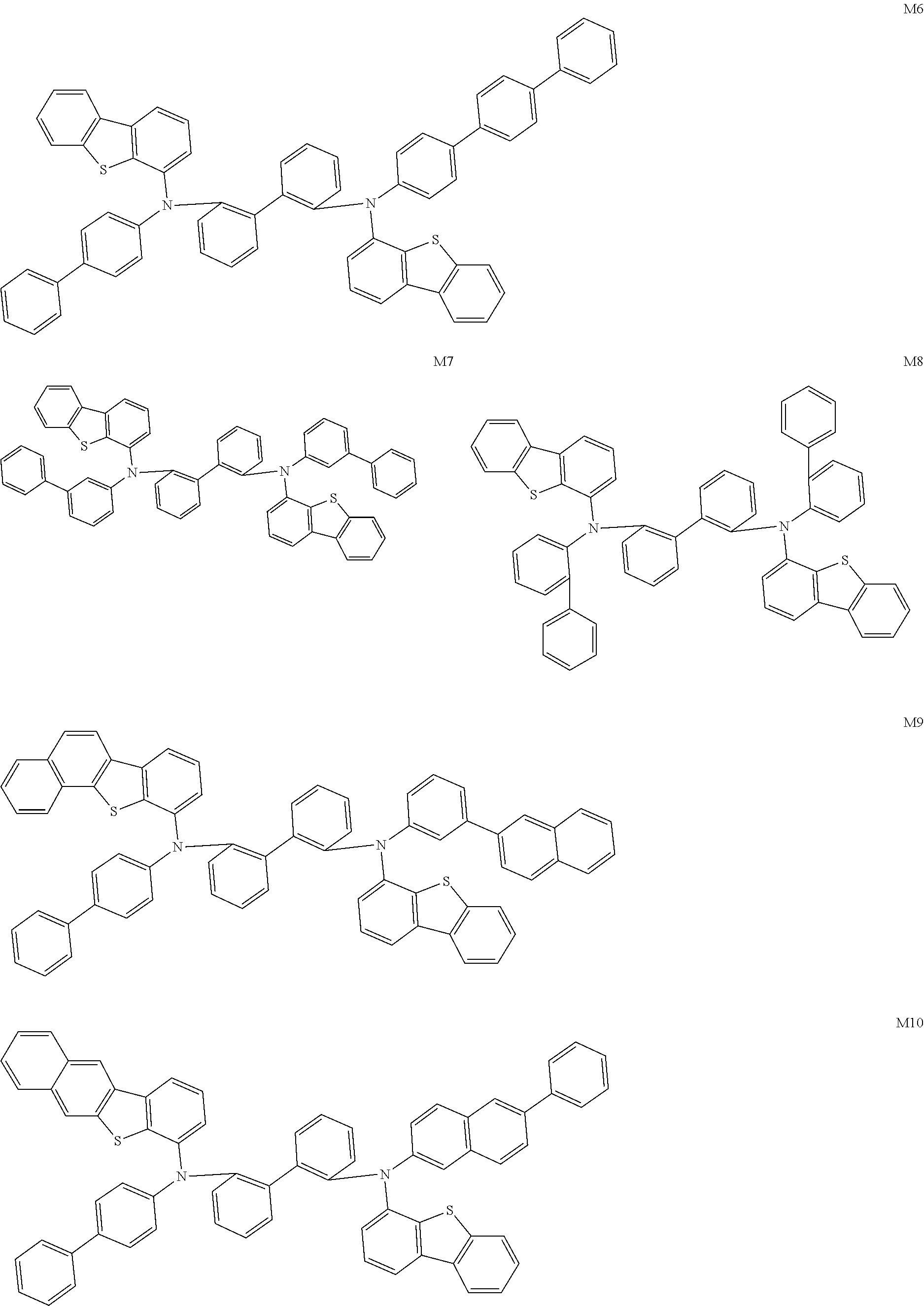

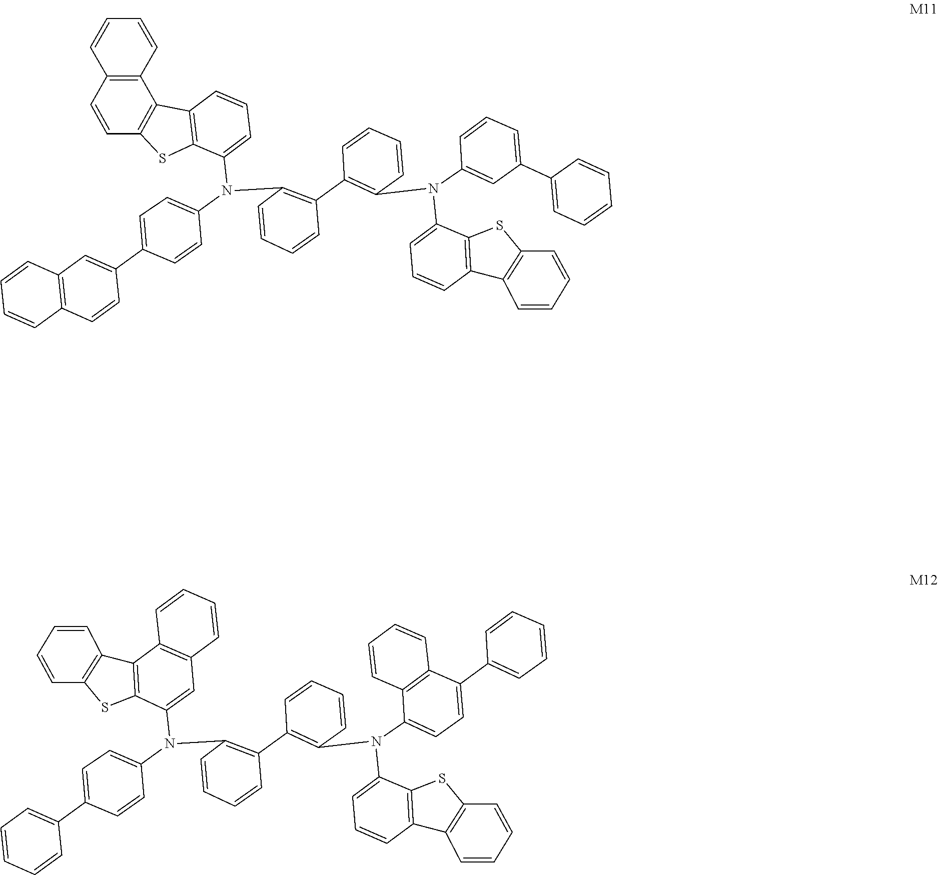 [SMILES: c1ccc(-c2cccc(N(c3ccccc3-c3ccccc3N(c3ccc(-c4ccc5ccccc5c4)cc3)c3cccc4c3sc3ccc5ccccc5c34)c3cccc4c3sc3ccccc34)c2)cc1 ; c1ccc(-c2ccc(N(c3ccccc3-c3ccccc3N(c3ccc(-c4ccccc4)c4ccccc34)c3cccc4c3sc3ccccc34)c3cc4ccccc4c4c3sc3ccccc34)cc2)cc1]

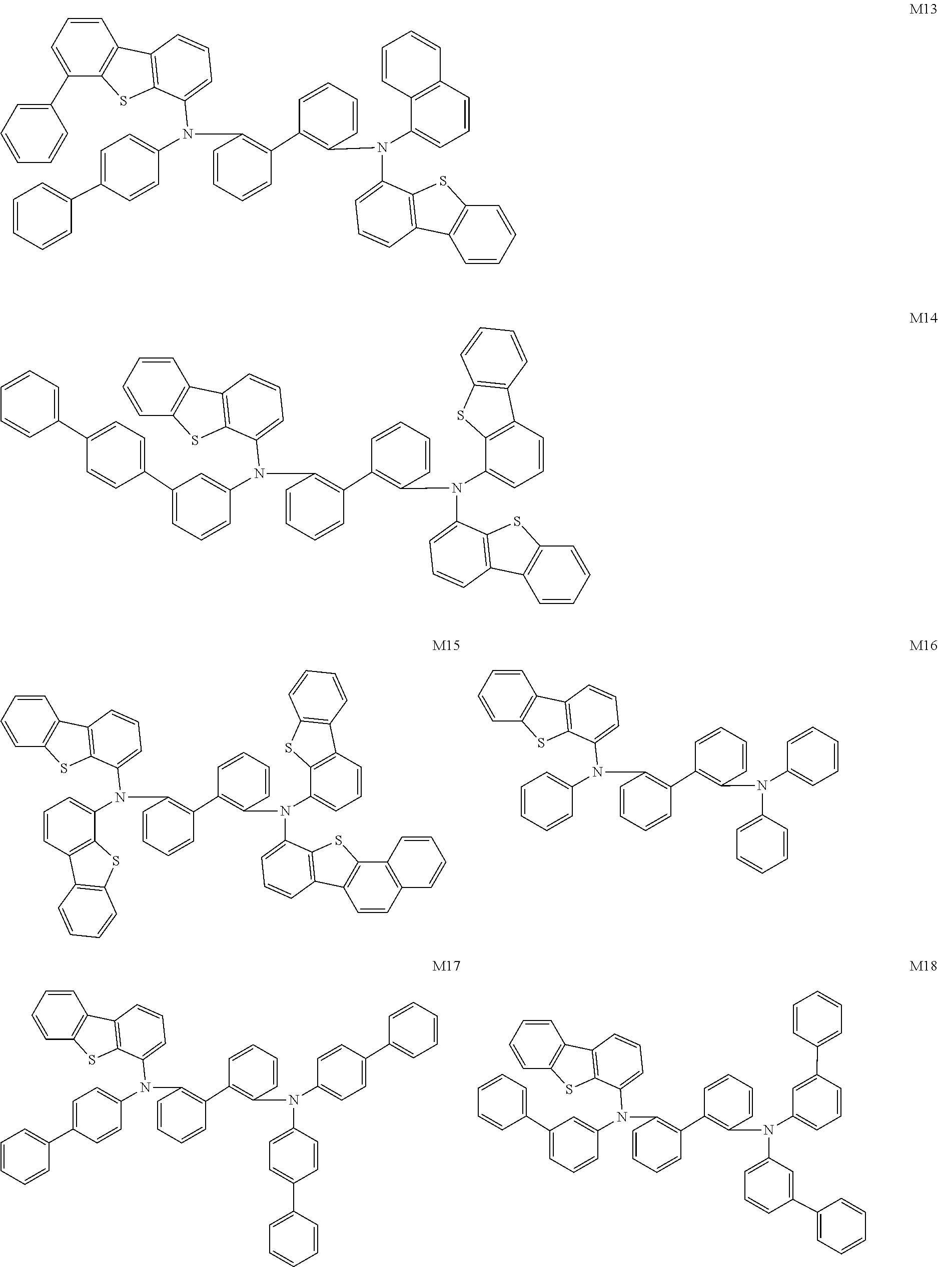

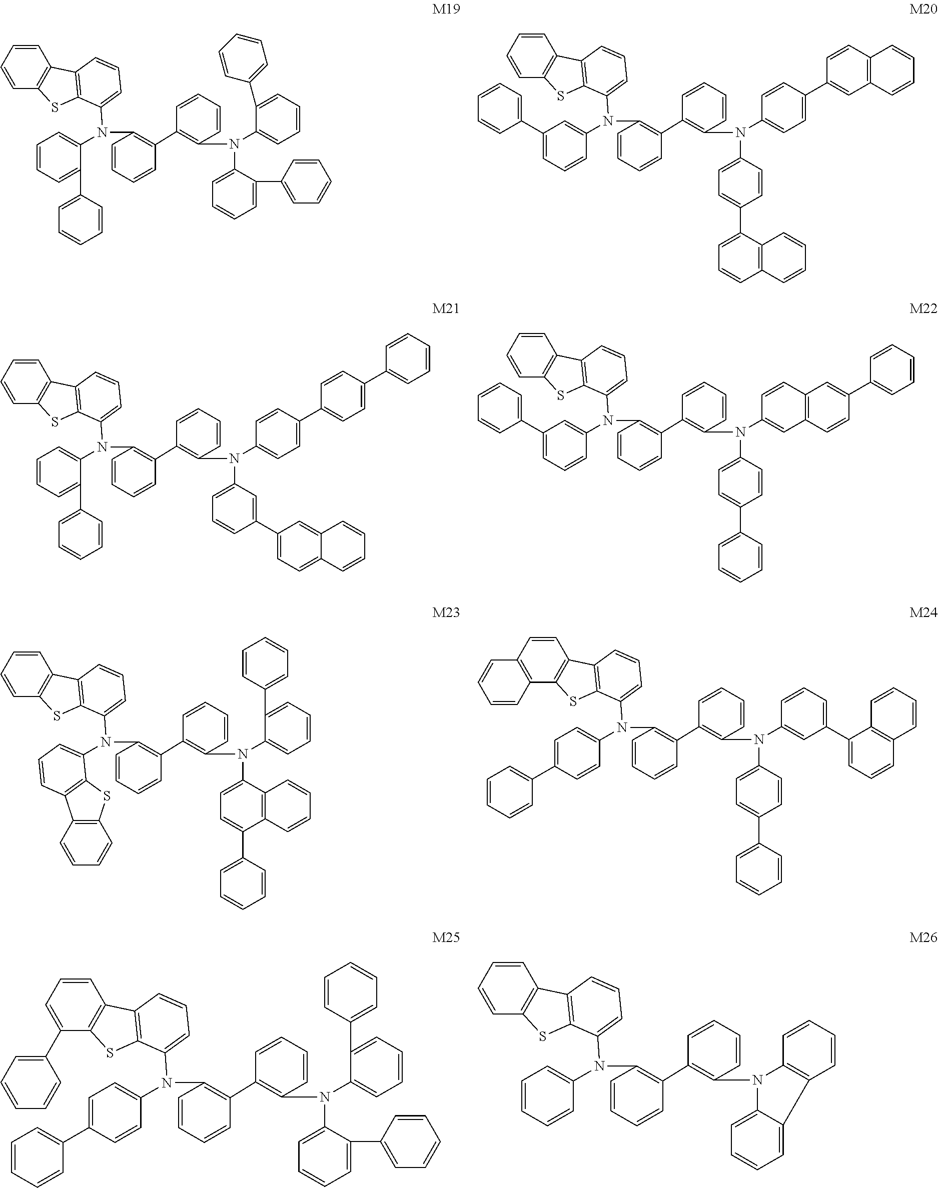

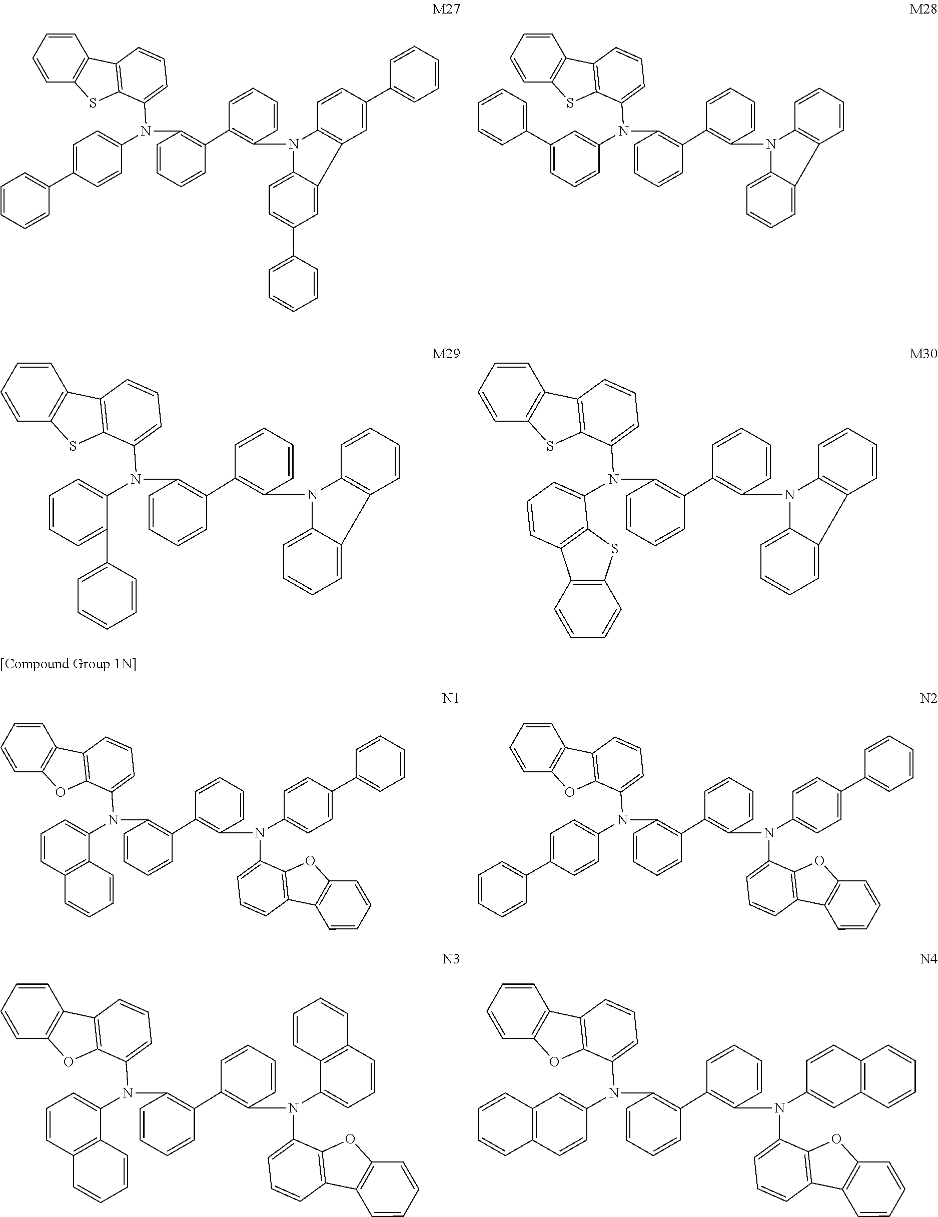

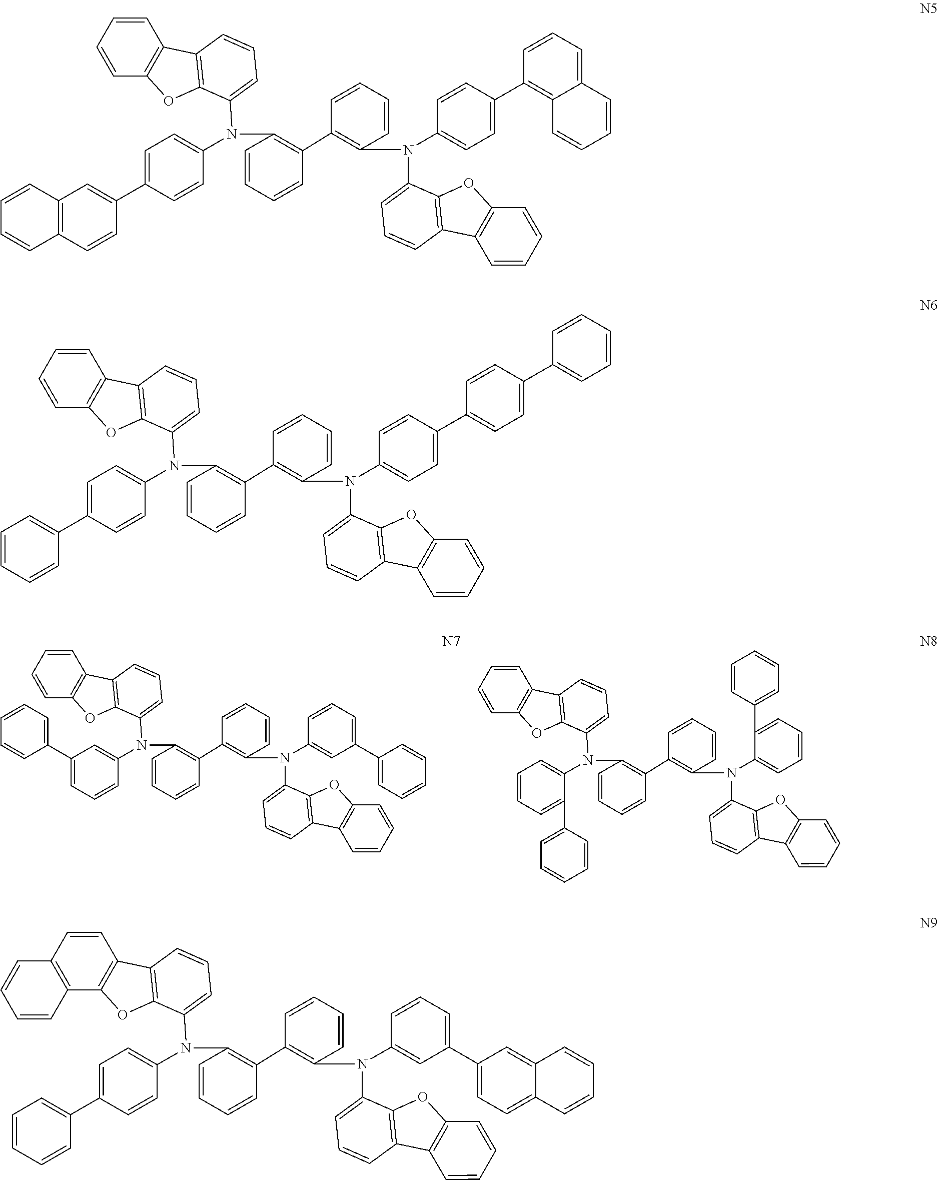

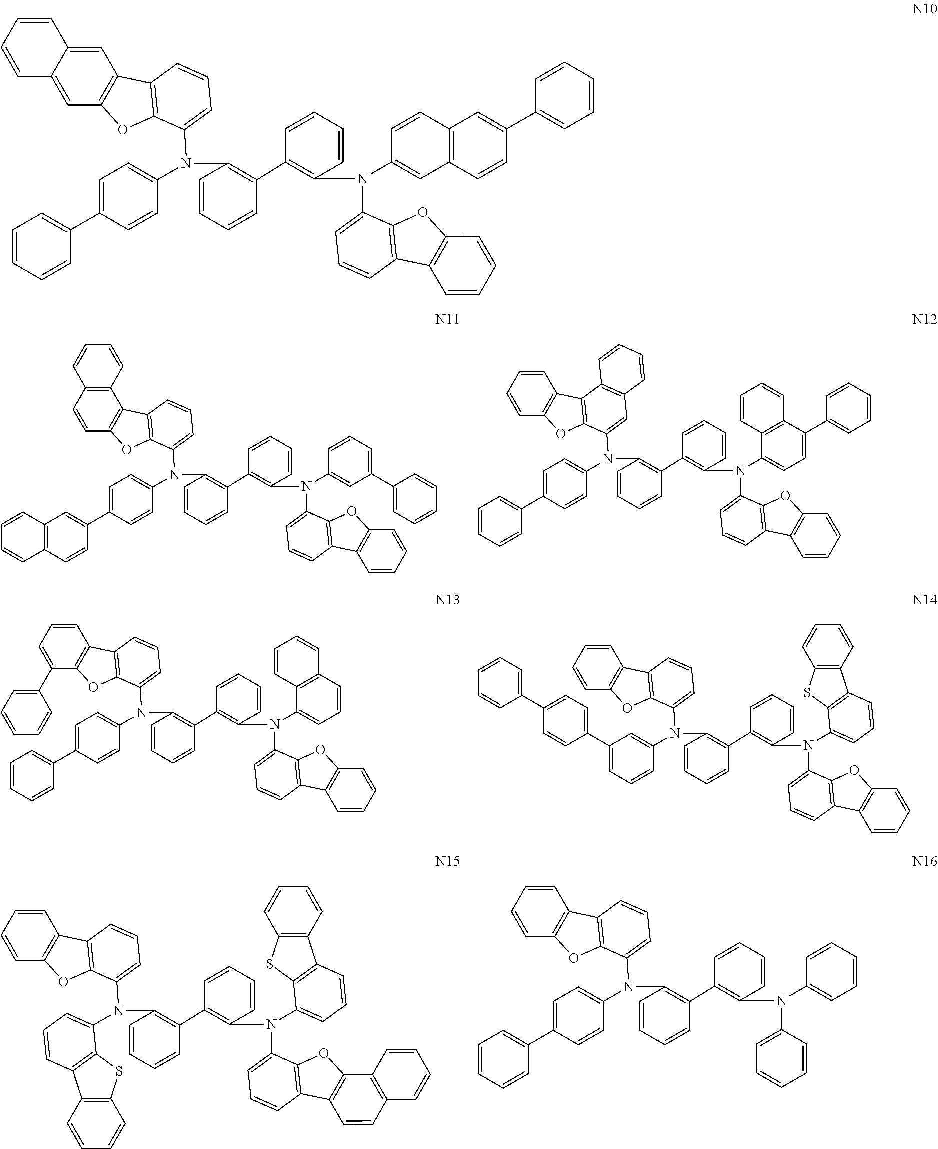

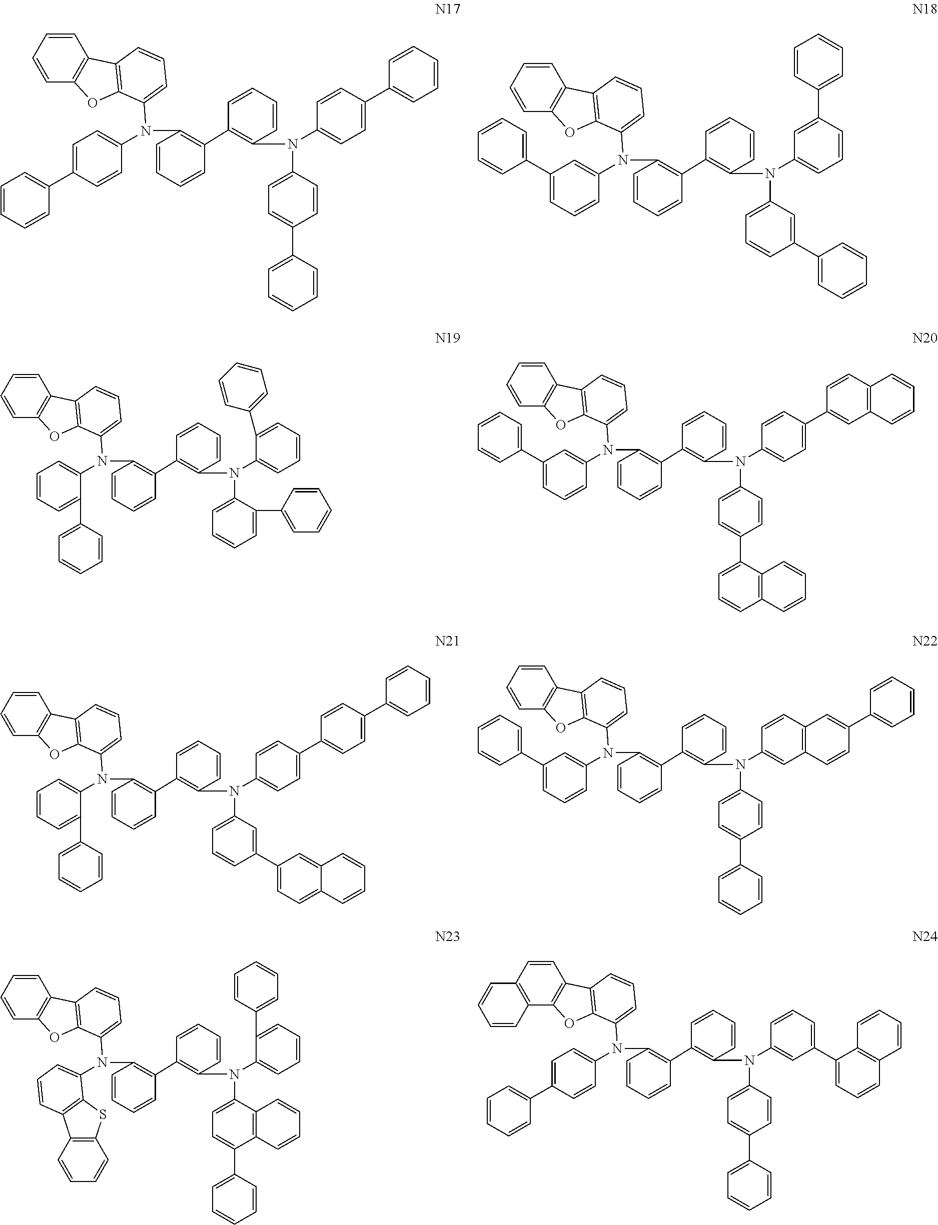

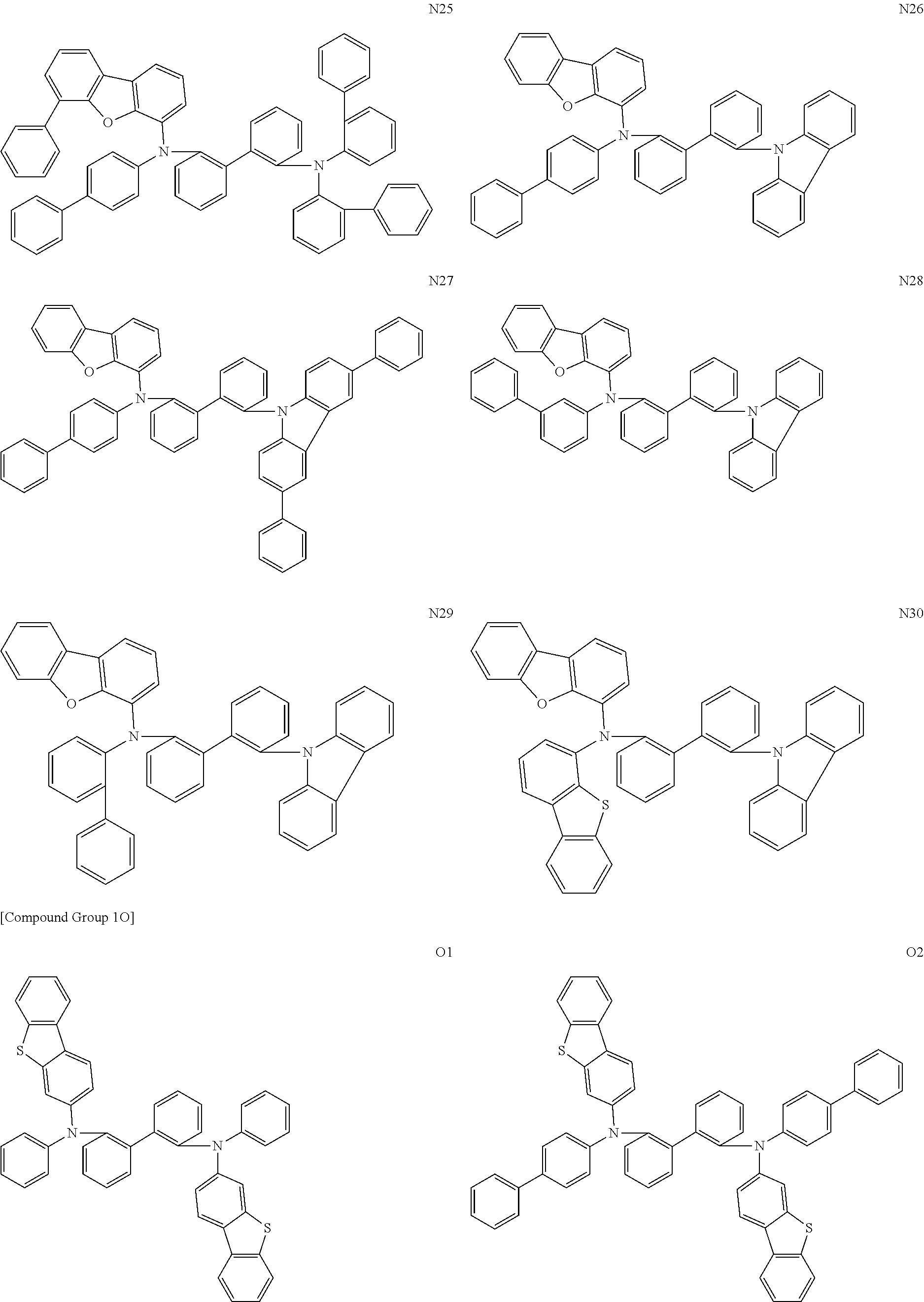

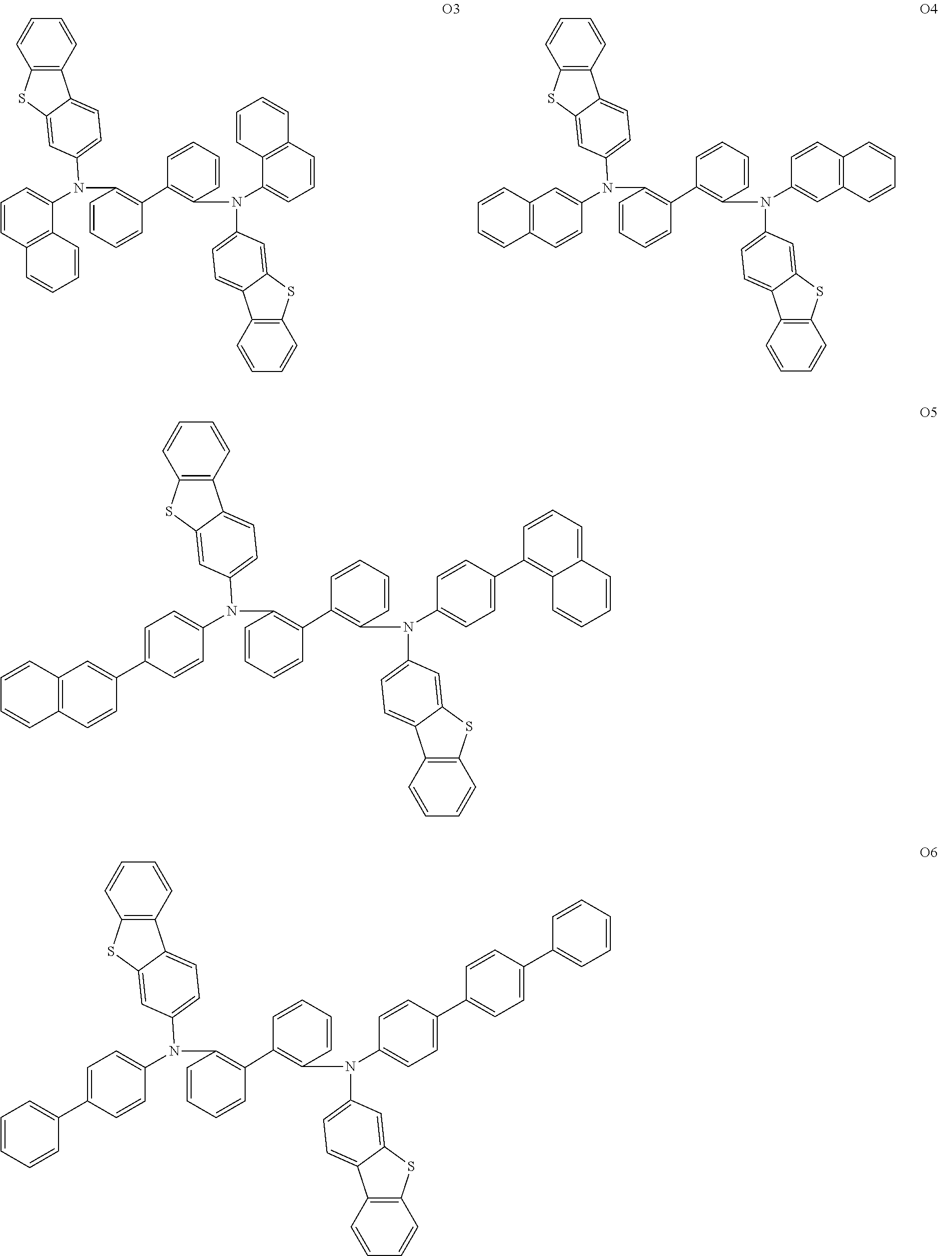 [SMILES: c1ccc(N(c2ccc3c(c2)sc2ccccc23)c2cccc3ccccc23)c(-c2ccccc2N(c2ccc3c(c2)sc2ccccc23)c2cccc3ccccc23)c1 ; c1ccc(N(c2ccc3ccccc3c2)c2ccc3c(c2)sc2ccccc23)c(-c2ccccc2N(c2ccc3ccccc3c2)c2ccc3c(c2)sc2ccccc23)c1 ; c1ccc(N(c2ccc(-c3ccc4ccccc4c3)cc2)c2ccc3c(c2)sc2ccccc23)c(-c2ccccc2N(c2ccc(-c3cccc4ccccc34)cc2)c2ccc3c(c2)sc2ccccc23)c1 ; c1ccc(-c2ccc(-c3ccc(N(c4ccc5c(c4)sc4ccccc45)c4ccccc4-c4ccccc4N(c4ccc(-c5ccccc5)cc4)c4ccc5c(c4)sc4ccccc45)cc3)cc2)cc1]

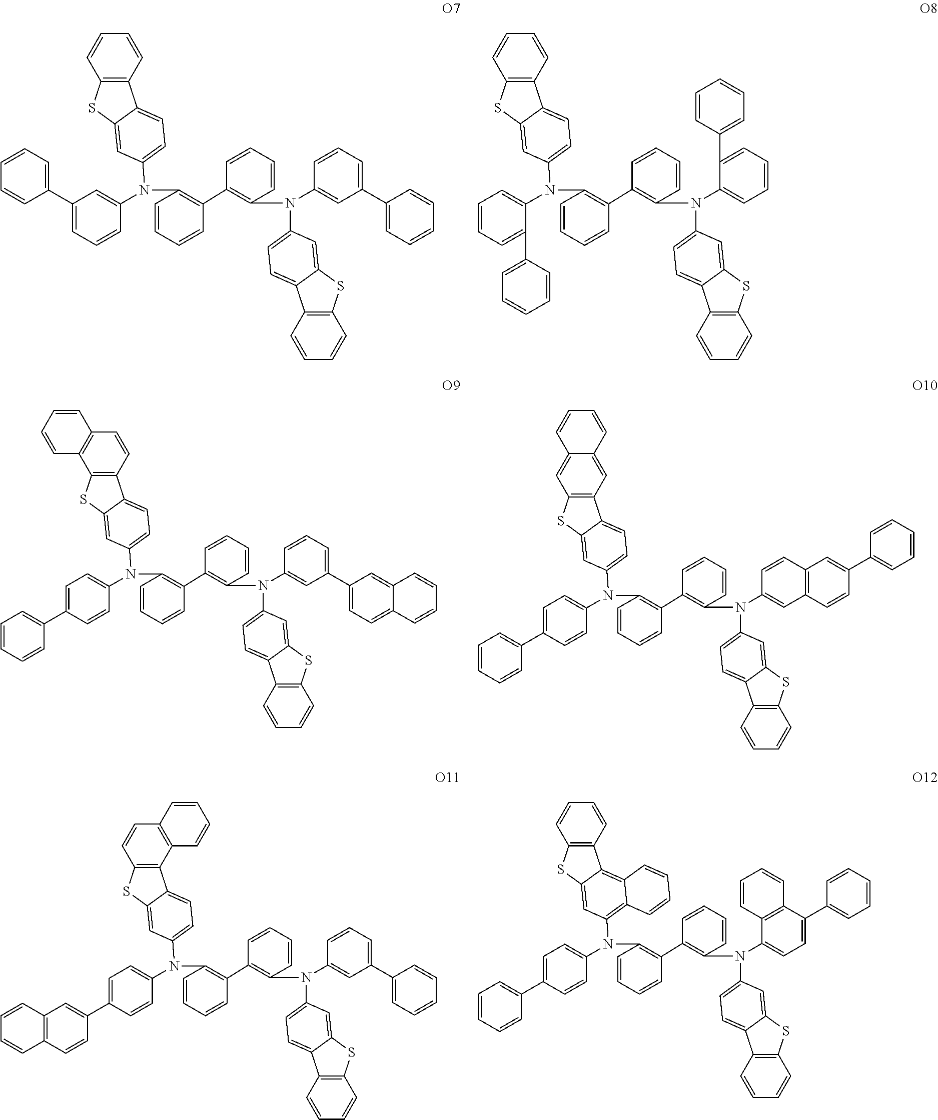

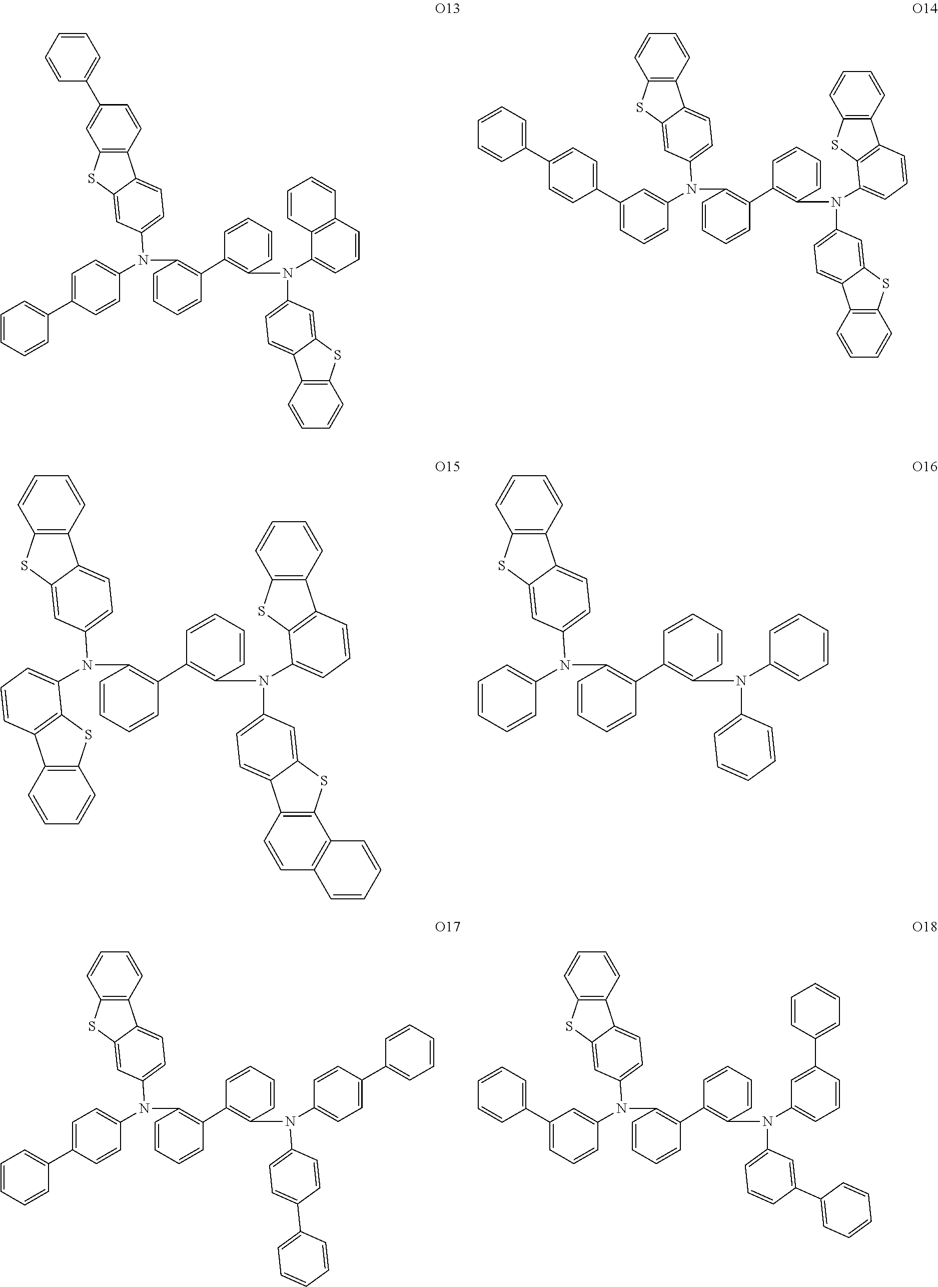

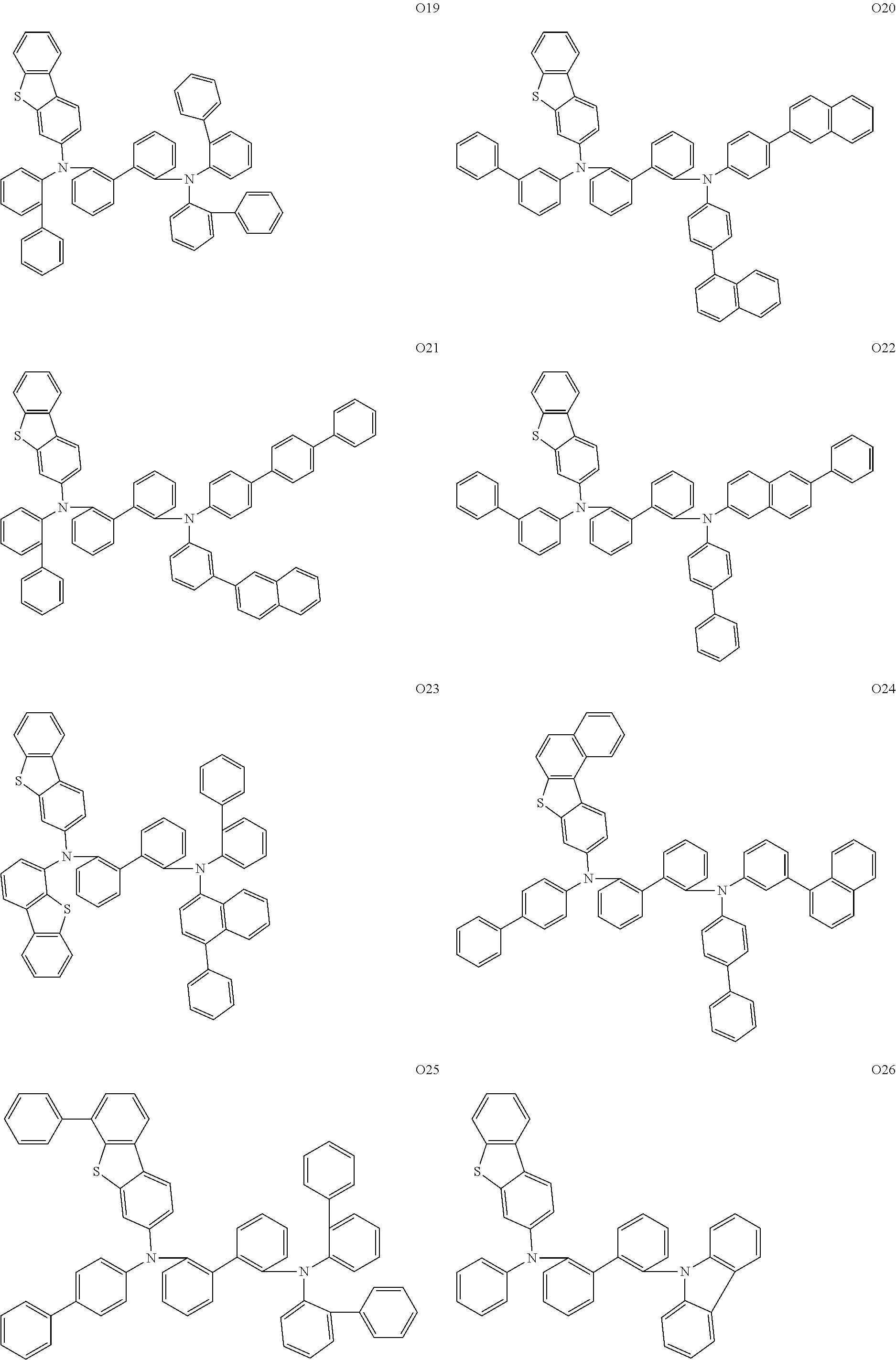

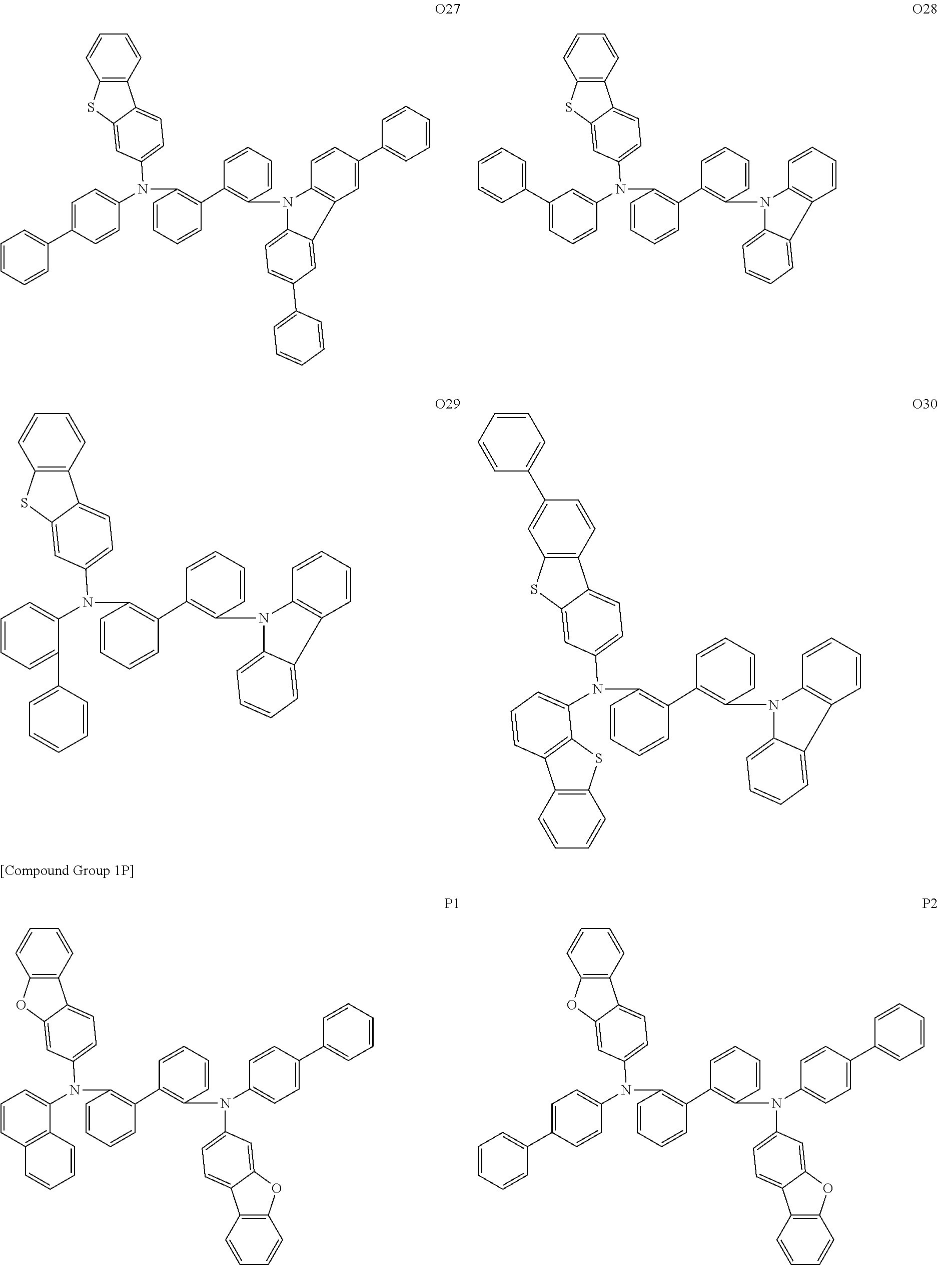

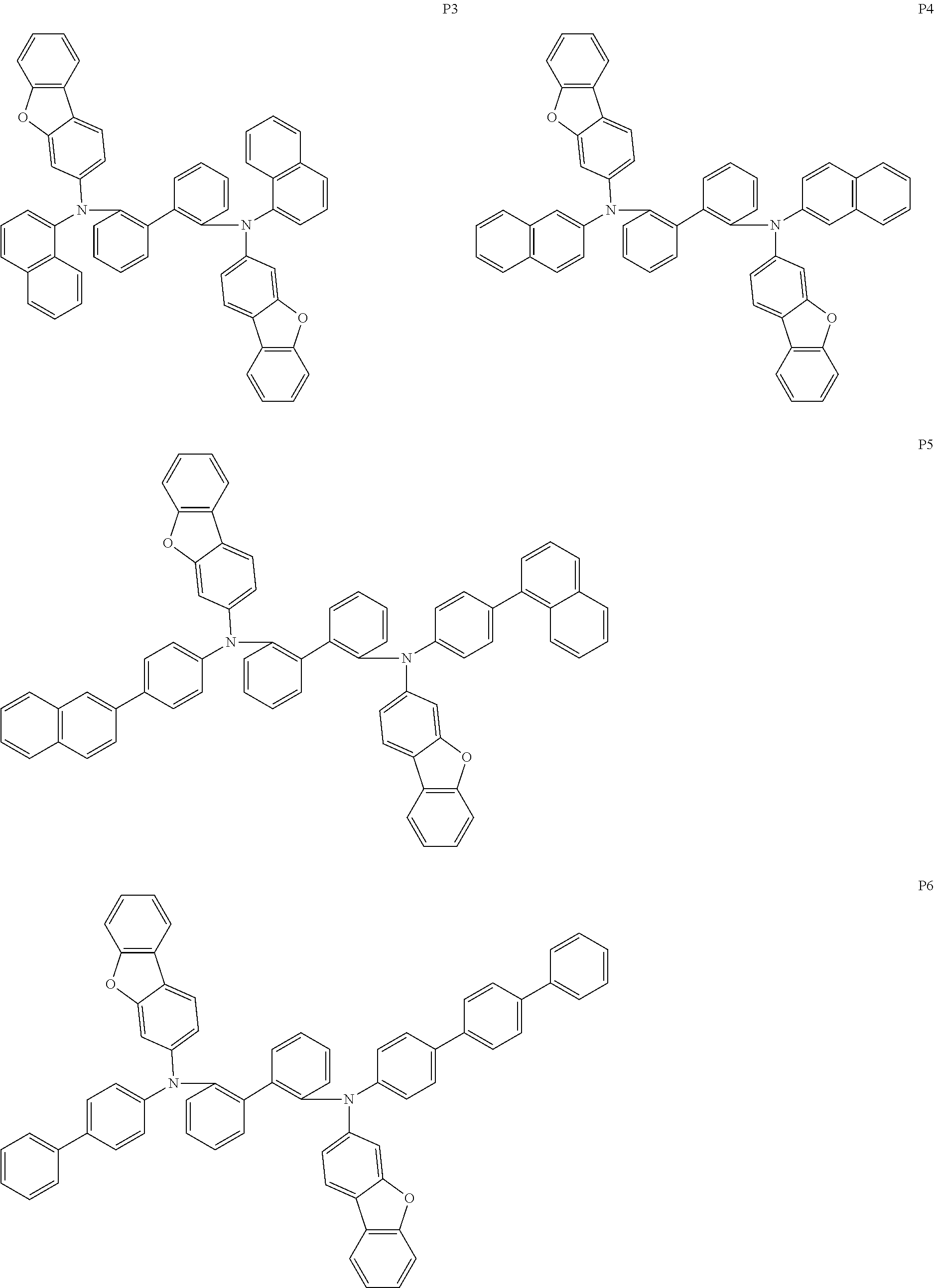 [SMILES: c1ccc(N(c2ccc3c(c2)oc2ccccc23)c2cccc3ccccc23)c(-c2ccccc2N(c2ccc3c(c2)oc2ccccc23)c2cccc3ccccc23)c1 ; c1ccc(N(c2ccc3ccccc3c2)c2ccc3c(c2)oc2ccccc23)c(-c2ccccc2N(c2ccc3ccccc3c2)c2ccc3c(c2)oc2ccccc23)c1 ; c1ccc(N(c2ccc(-c3ccc4ccccc4c3)cc2)c2ccc3c(c2)oc2ccccc23)c(-c2ccccc2N(c2ccc(-c3cccc4ccccc34)cc2)c2ccc3c(c2)oc2ccccc23)c1 ; c1ccc(-c2ccc(-c3ccc(N(c4ccc5c(c4)oc4ccccc45)c4ccccc4-c4ccccc4N(c4ccc(-c5ccccc5)cc4)c4ccc5c(c4)oc4ccccc45)cc3)cc2)cc1]

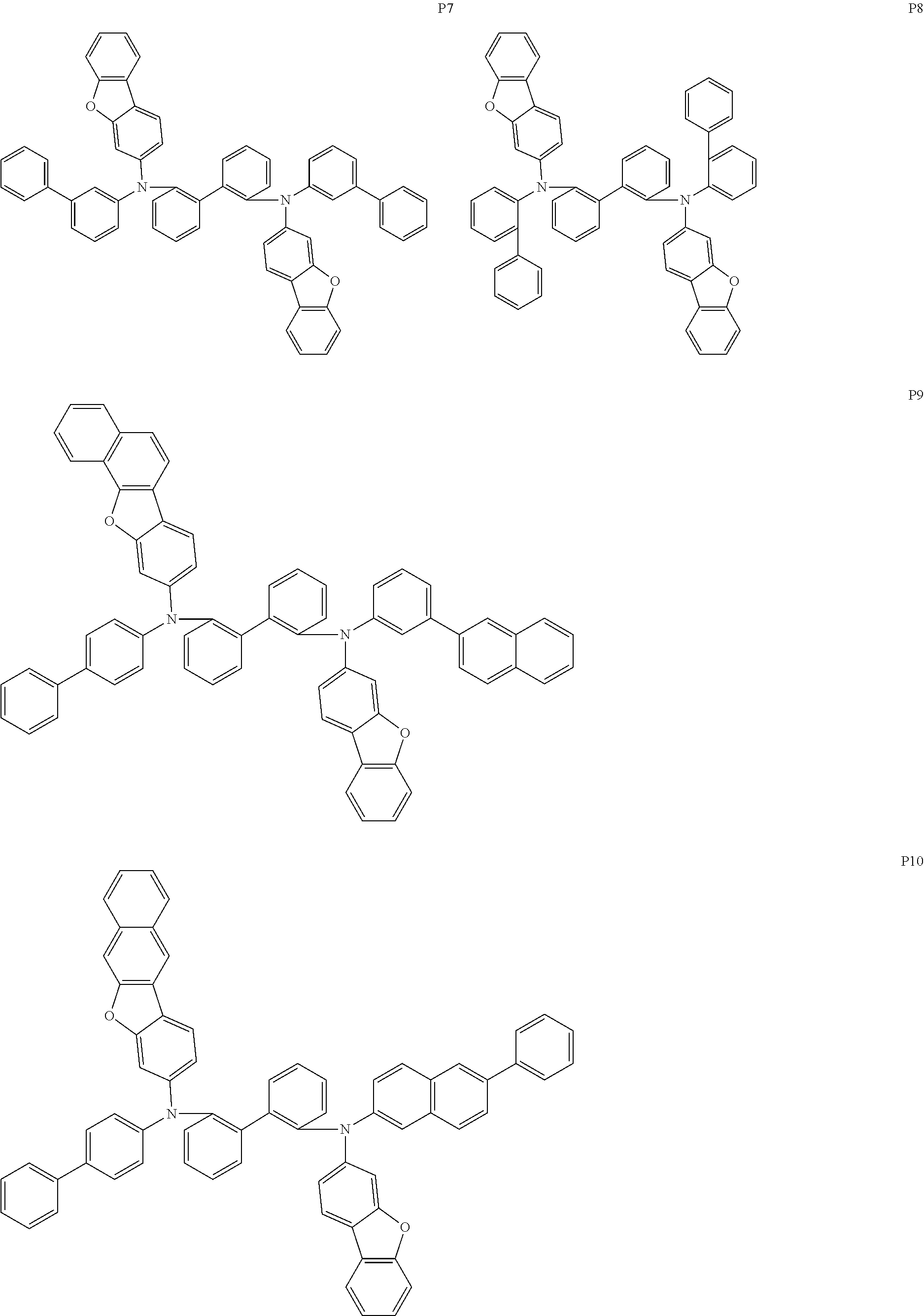

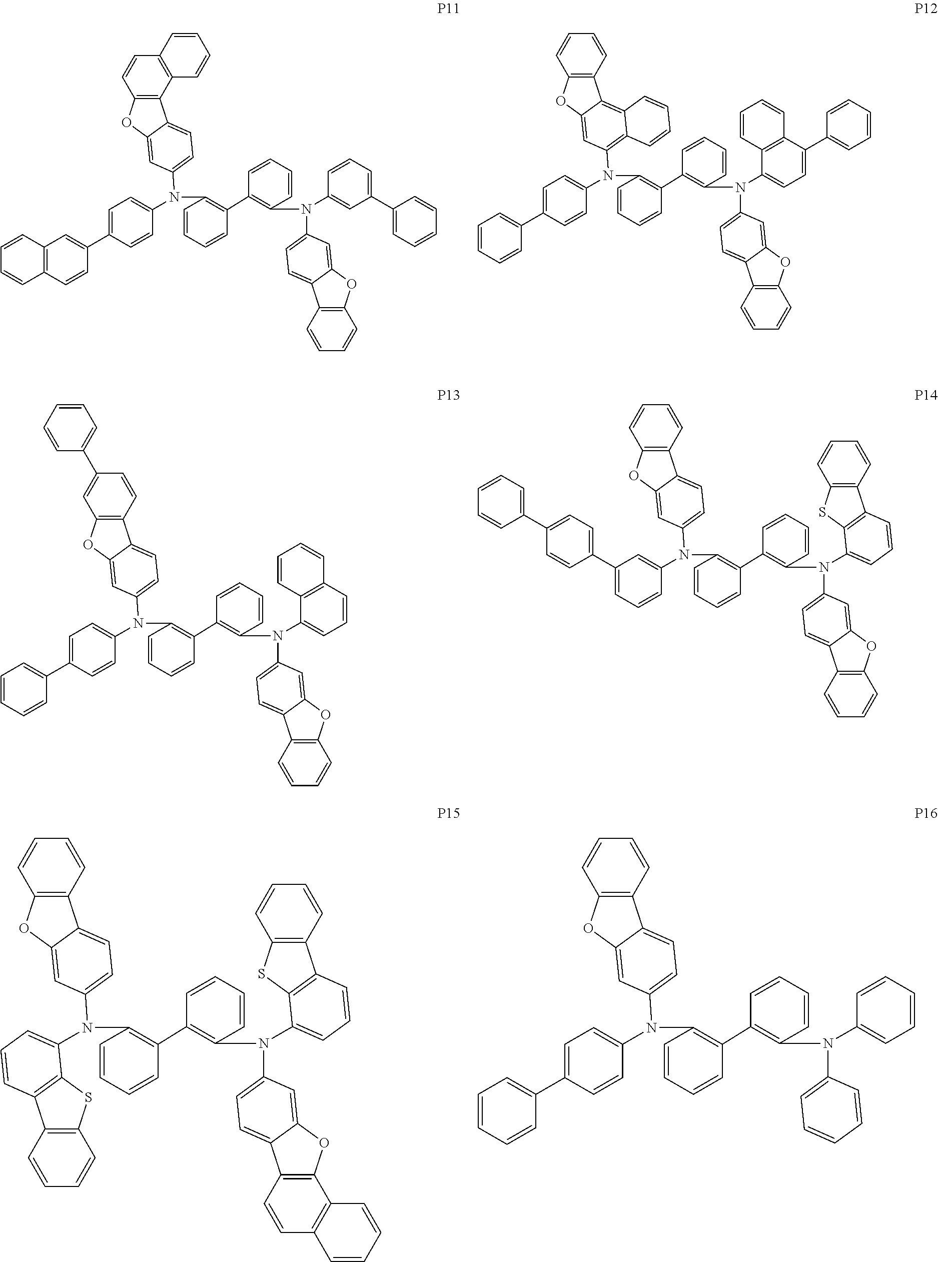

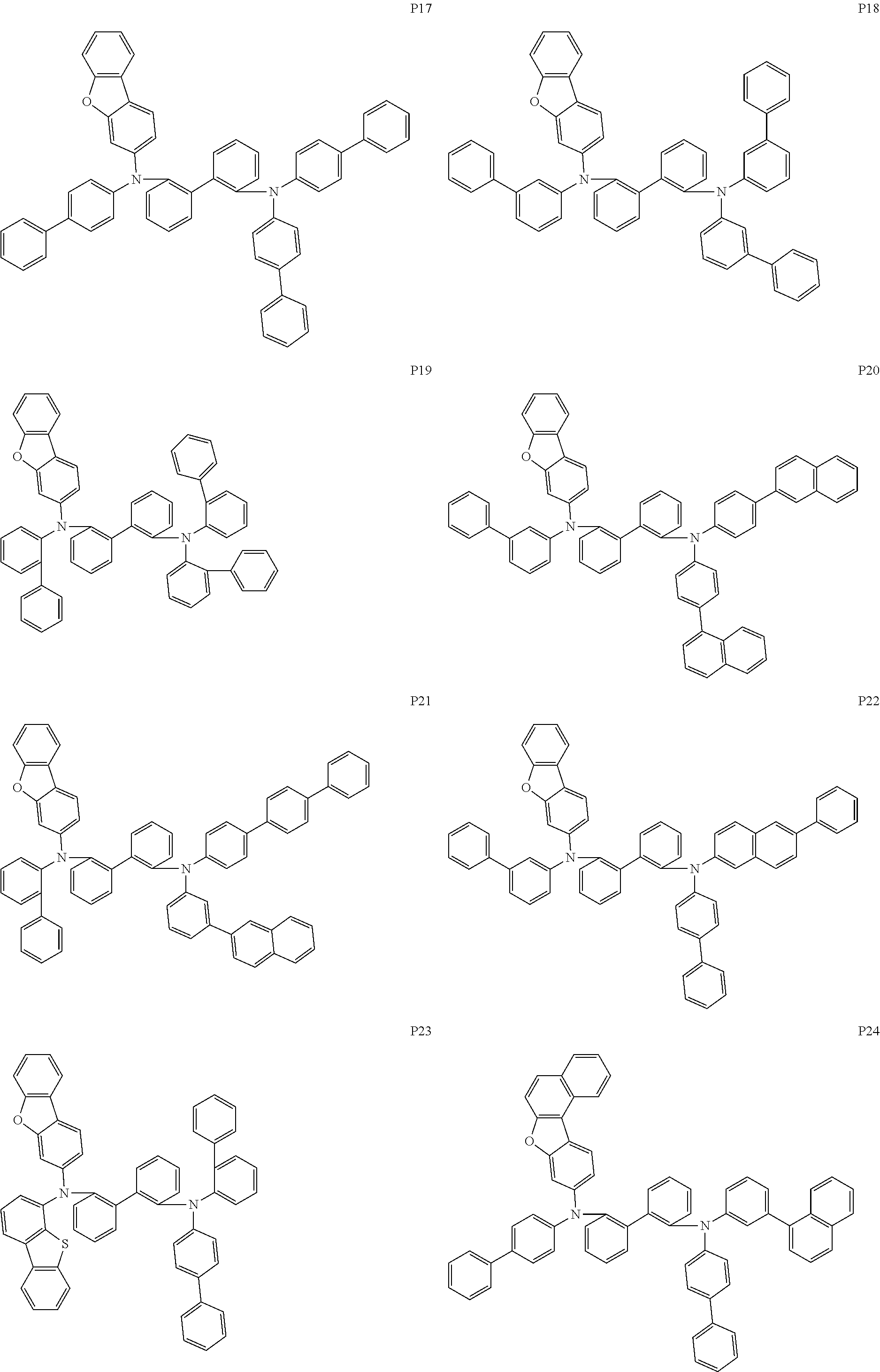

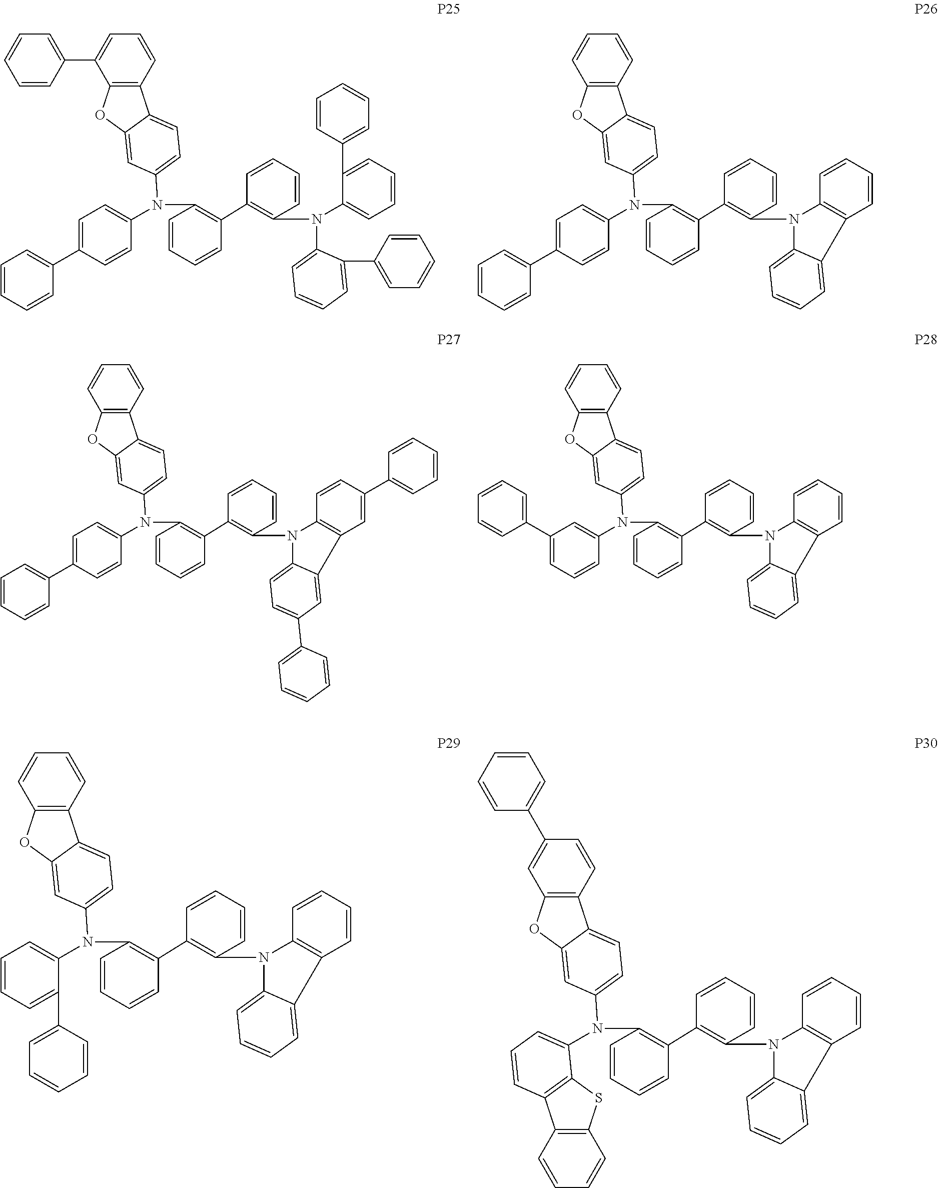

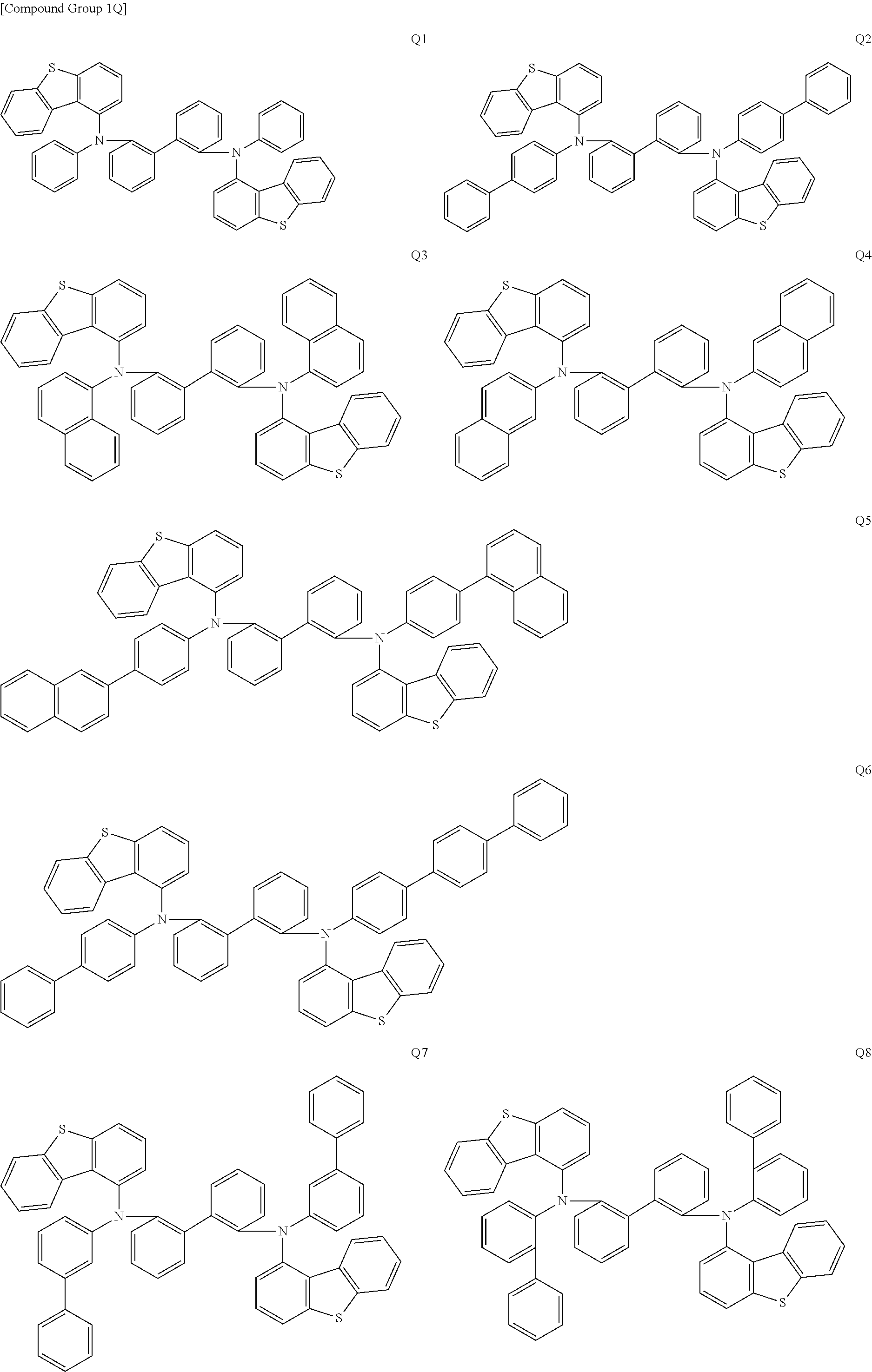

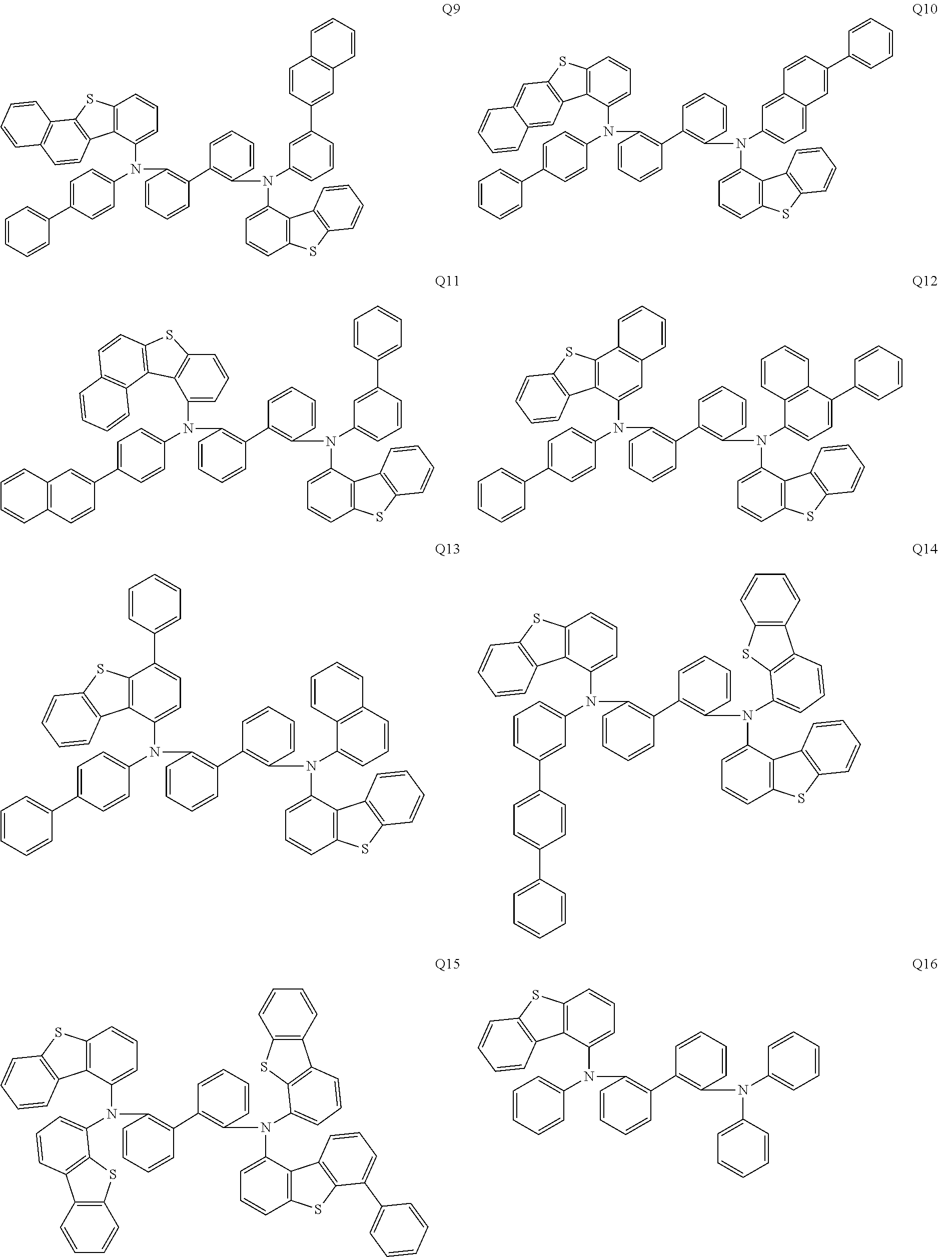

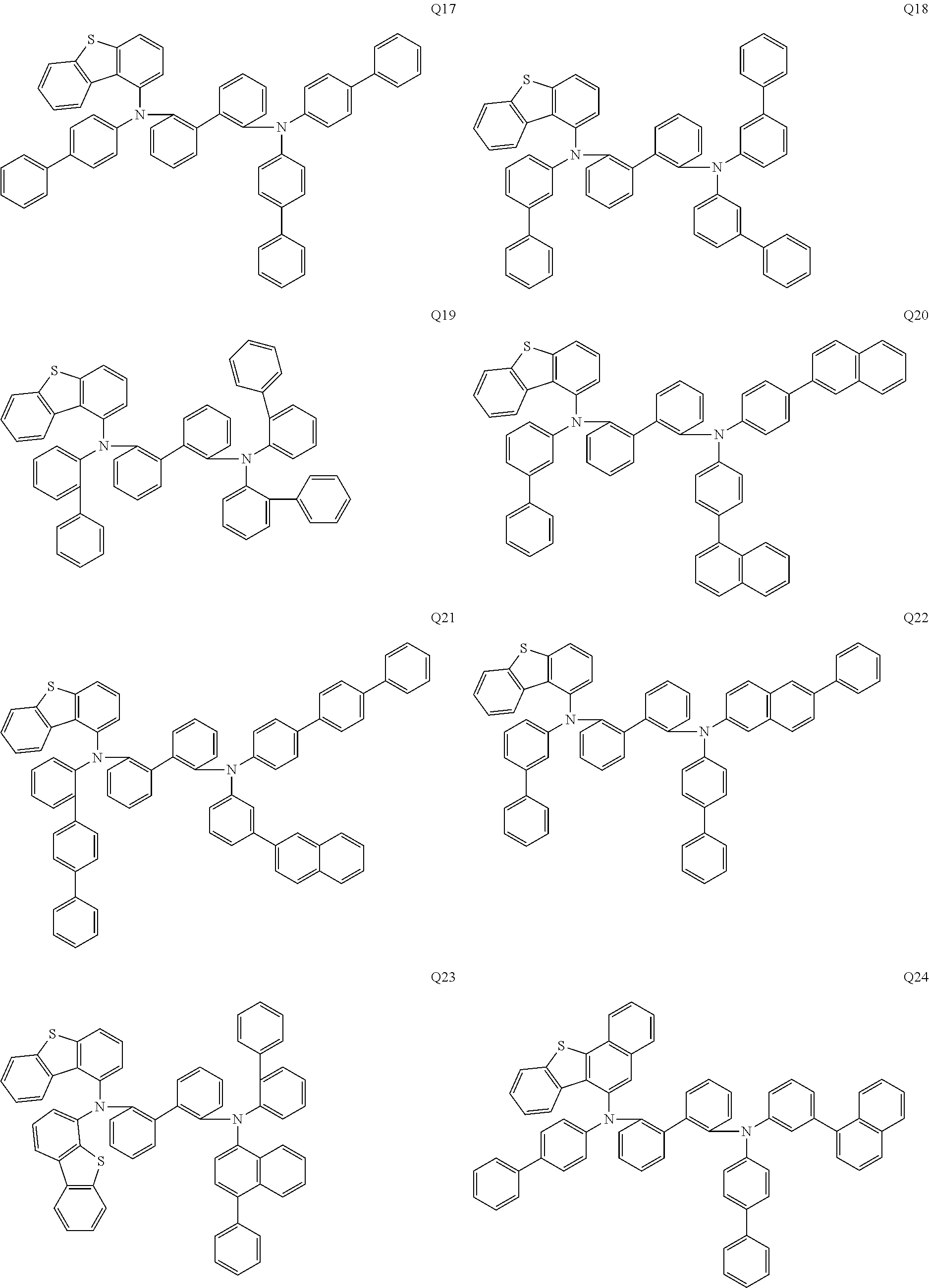

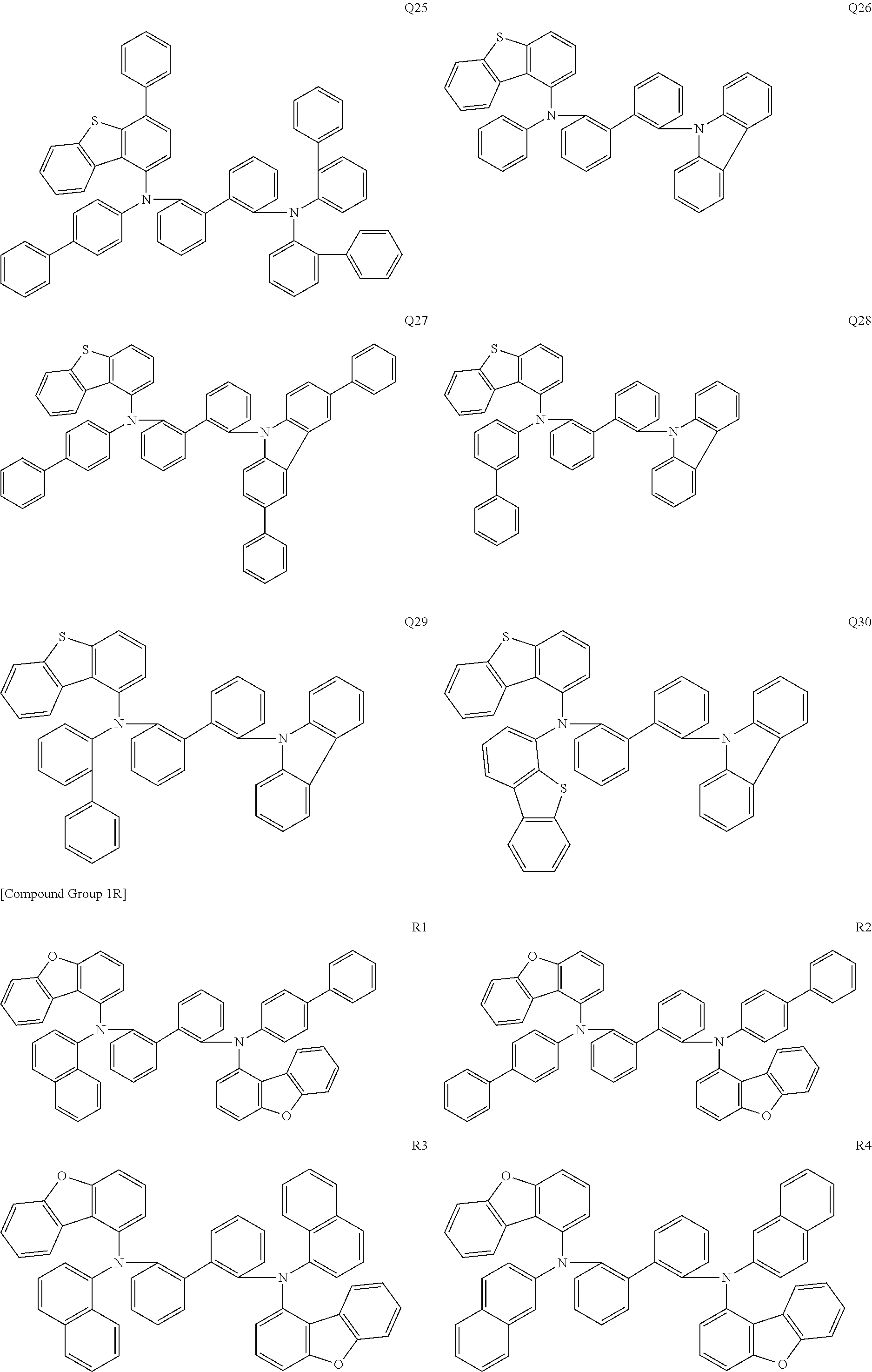

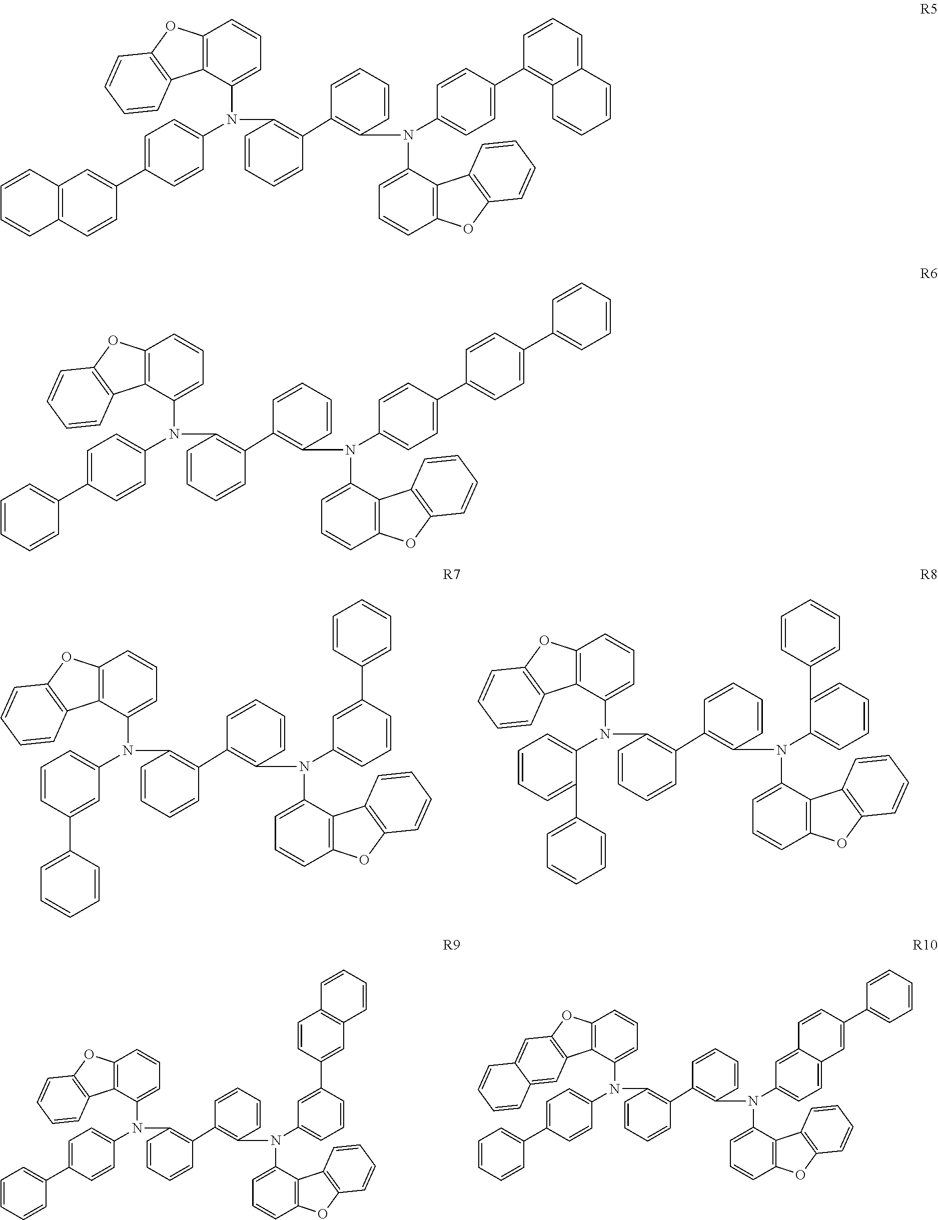

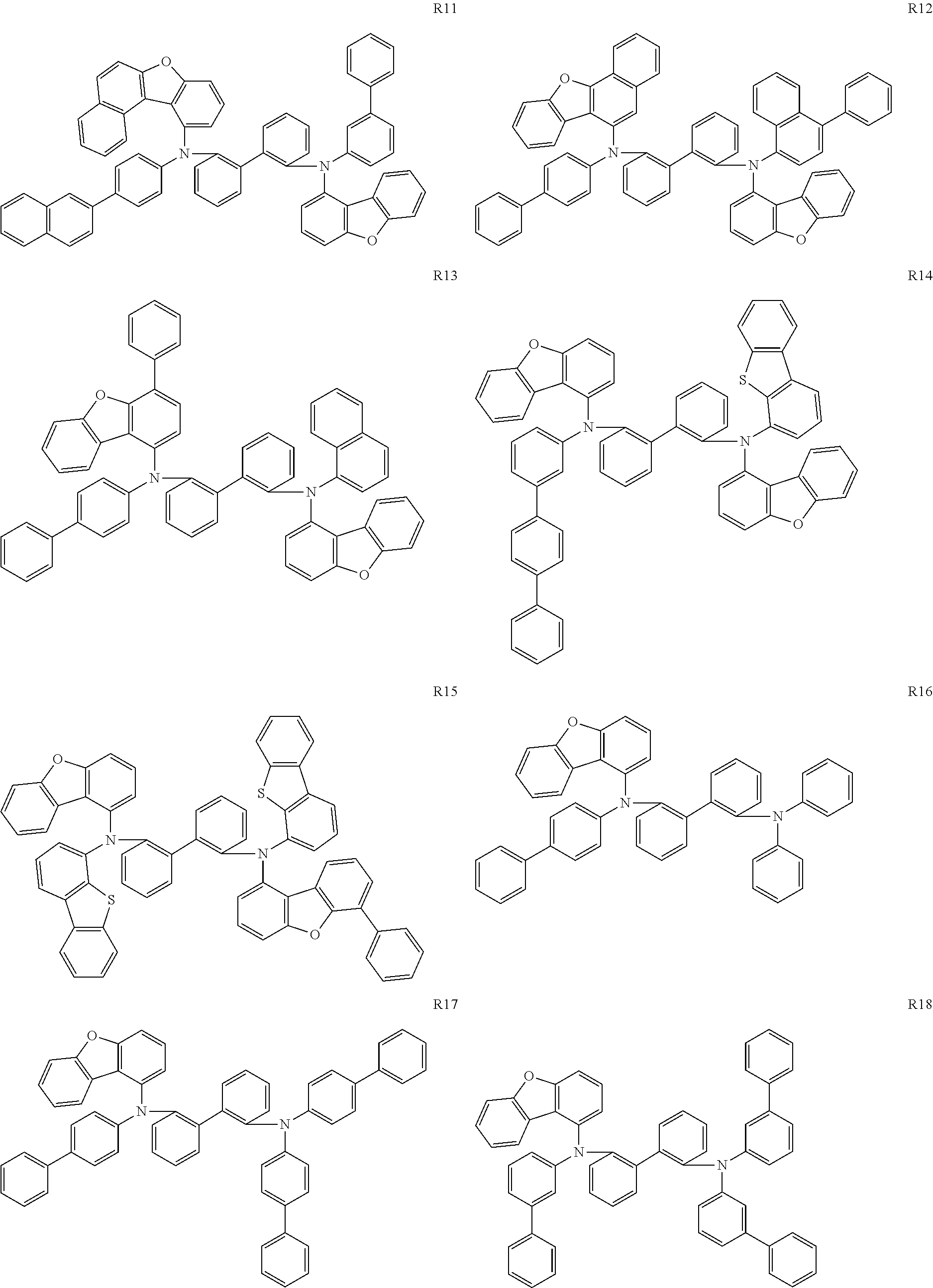

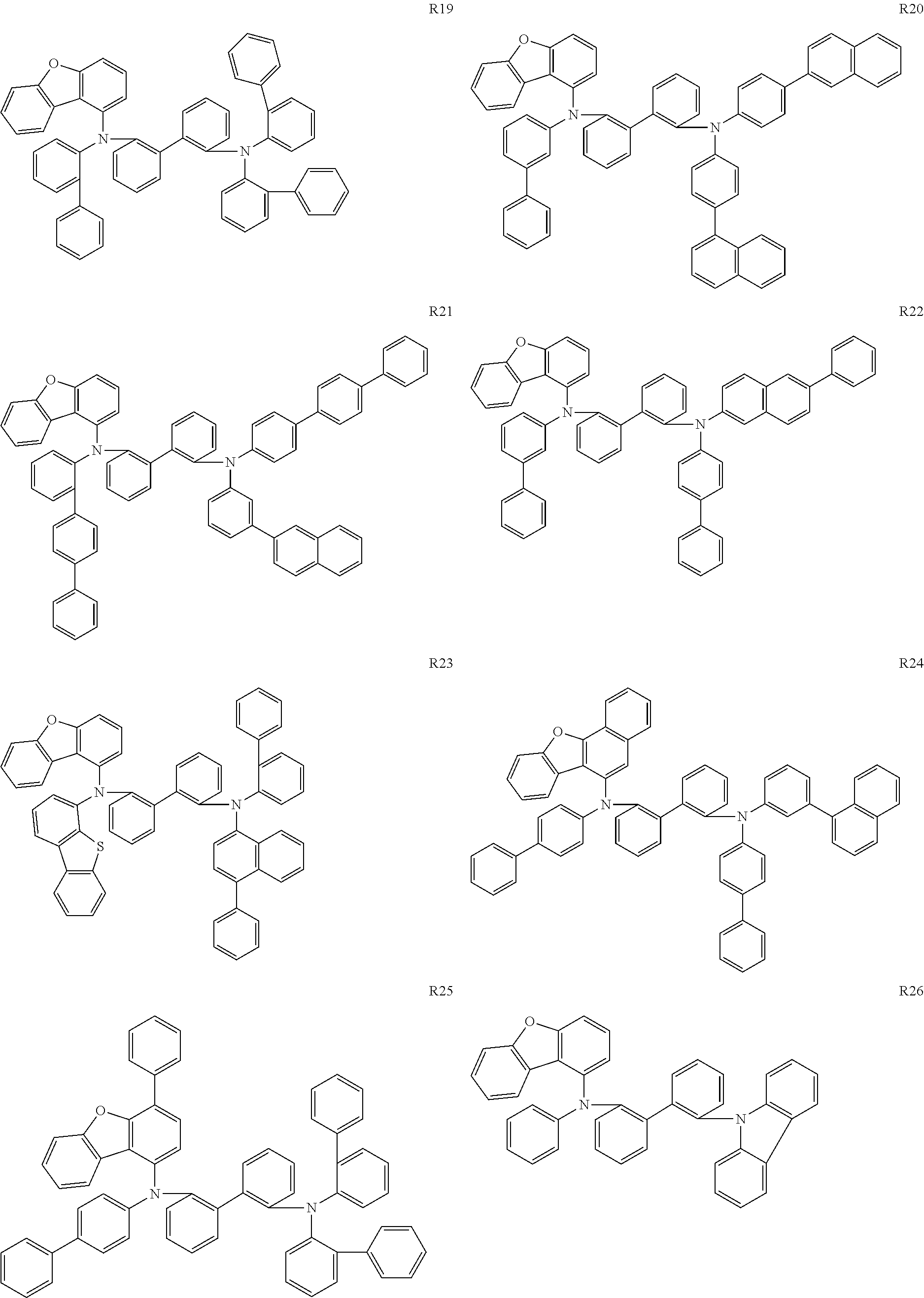

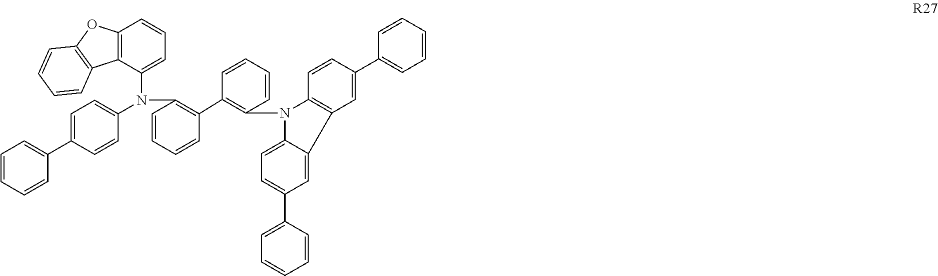 [SMILES: c1ccc(-c2ccc(N(c3ccccc3-c3ccccc3-n3c4ccc(-c5ccccc5)cc4c4cc(-c5ccccc5)ccc43)c3cccc4oc5ccccc5c34)cc2)cc1]

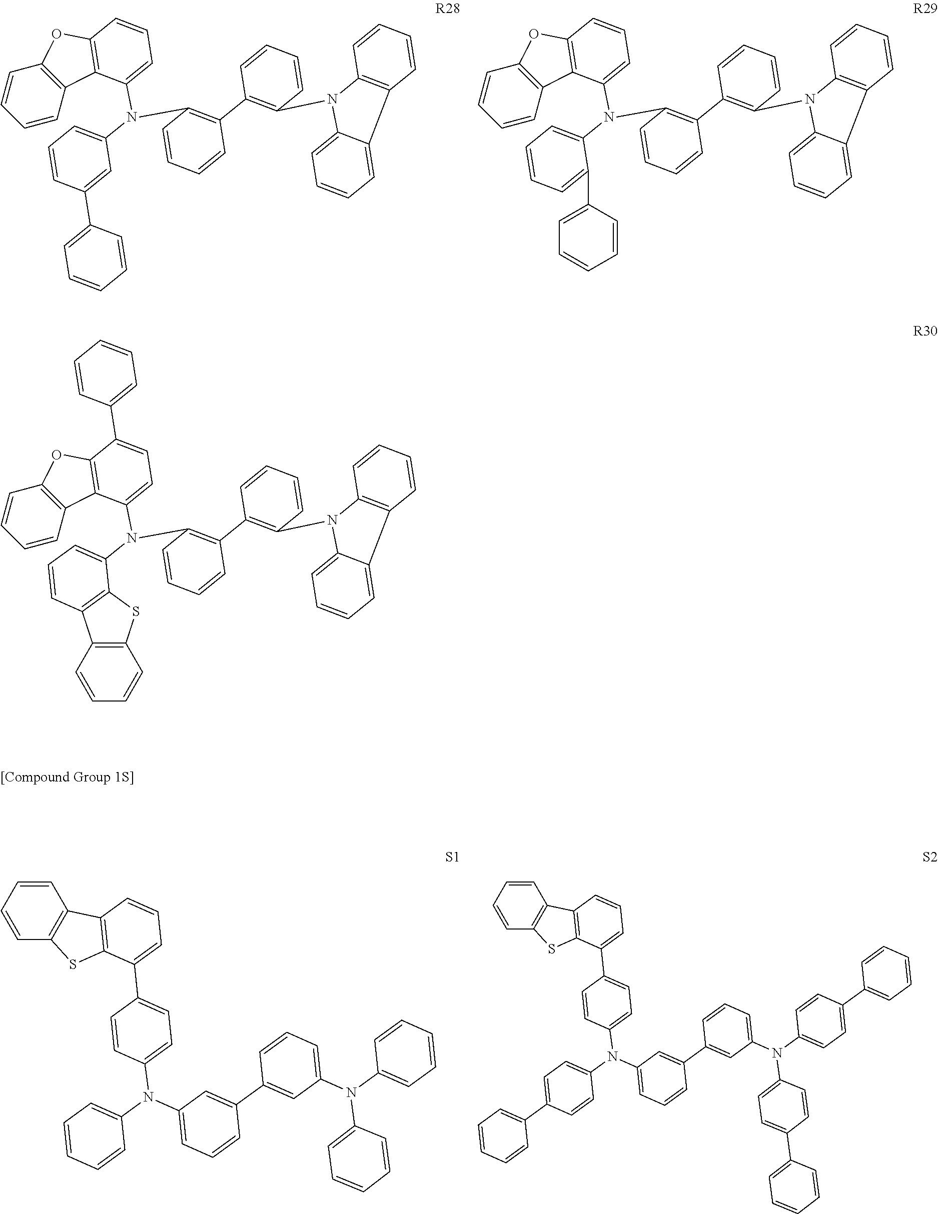 [SMILES: c1ccc(-c2cccc(N(c3ccccc3-c3ccccc3-n3c4ccccc4c4ccccc43)c3cccc4oc5ccccc5c34)c2)cc1 ; c1ccc(-c2ccccc2N(c2ccccc2-c2ccccc2-n2c3ccccc3c3ccccc32)c2cccc3oc4ccccc4c23)cc1 ; c1ccc(-c2ccc(N(c3ccccc3-c3ccccc3-n3c4ccccc4c4ccccc43)c3cccc4c3sc3ccccc34)c3c2oc2ccccc23)cc1 ; c1ccc(N(c2ccccc2)c2cccc(-c3cccc(N(c4ccccc4)c4ccc(-c5cccc6c5sc5ccccc56)cc4)c3)c2)cc1 ; c1ccc(-c2ccc(N(c3ccc(-c4ccccc4)cc3)c3cccc(-c4cccc(N(c5ccc(-c6ccccc6)cc5)c5ccc(-c6cccc7c6sc6ccccc67)cc5)c4)c3)cc2)cc1]

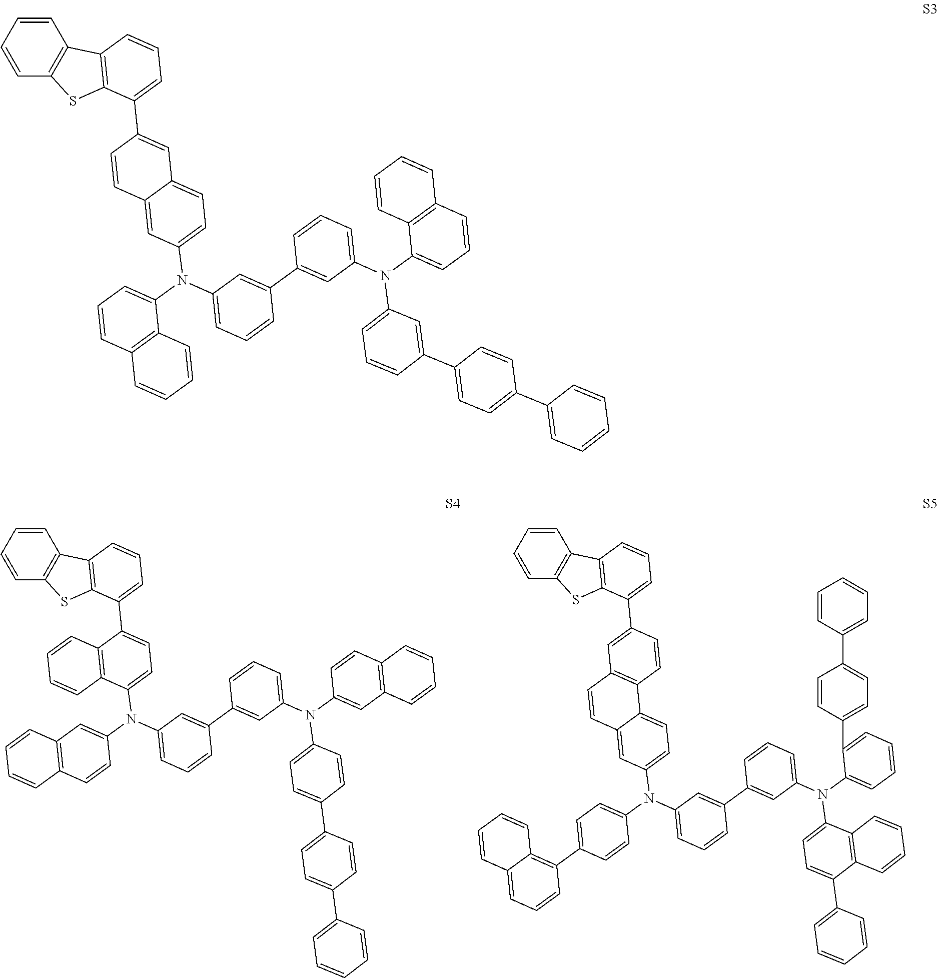 [SMILES: c1ccc(-c2ccc(-c3cccc(N(c4cccc(-c5cccc(N(c6ccc7cc(-c8cccc9c8sc8ccccc89)ccc7c6)c6cccc7ccccc67)c5)c4)c4cccc5ccccc45)c3)cc2)cc1 ; c1ccc(-c2ccc(-c3ccc(N(c4cccc(-c5cccc(N(c6ccc7ccccc7c6)c6ccc(-c7cccc8c7sc7ccccc78)c7ccccc67)c5)c4)c4ccc5ccccc5c4)cc3)cc2)cc1 ; c1ccc(-c2ccc(-c3ccccc3N(c3cccc(-c4cccc(N(c5ccc(-c6cccc7ccccc67)cc5)c5ccc6c(ccc7cc(-c8cccc9c8sc8ccccc89)ccc76)c5)c4)c3)c3ccc(-c4ccccc4)c4ccccc34)cc2)cc1]

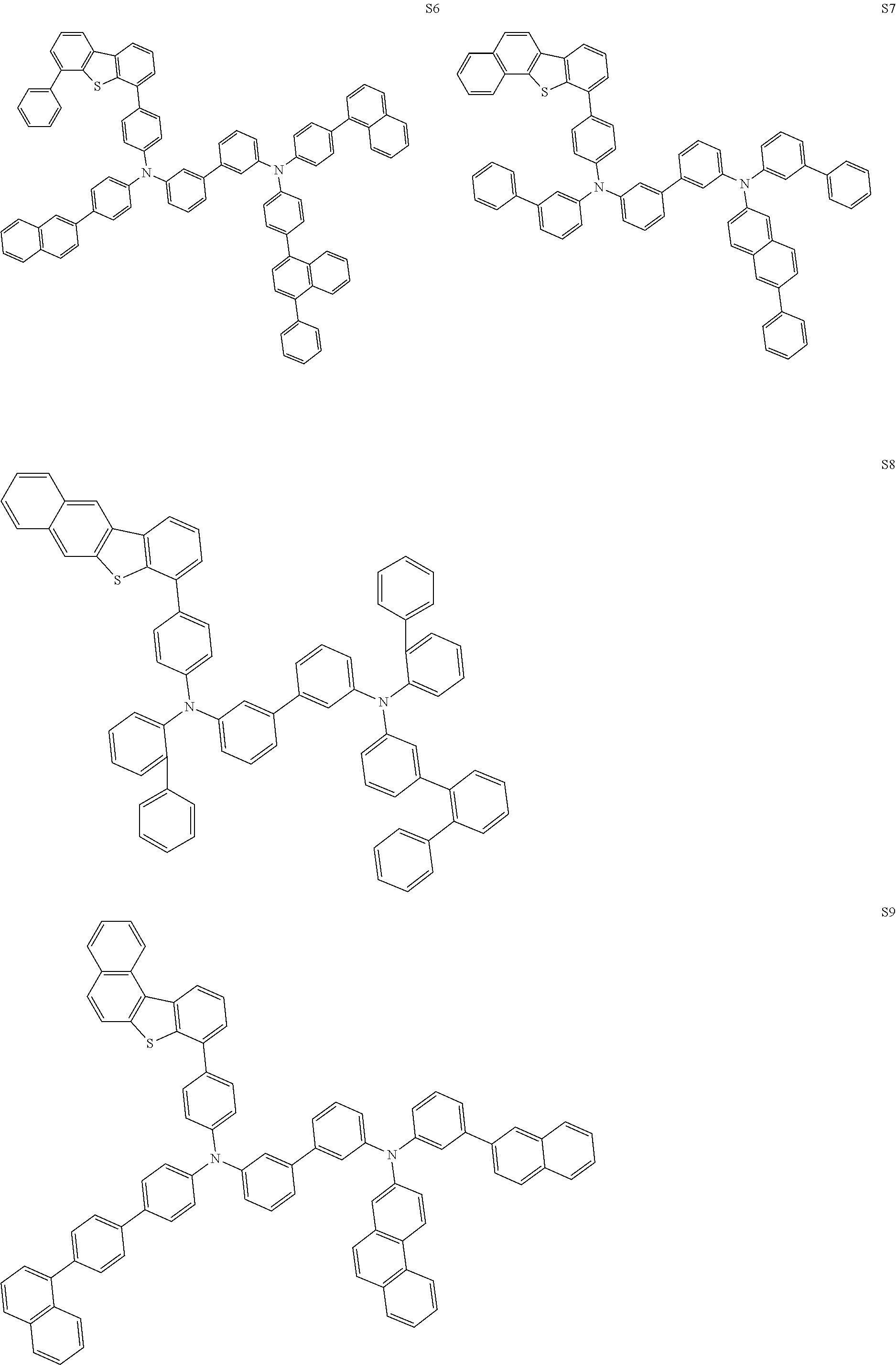

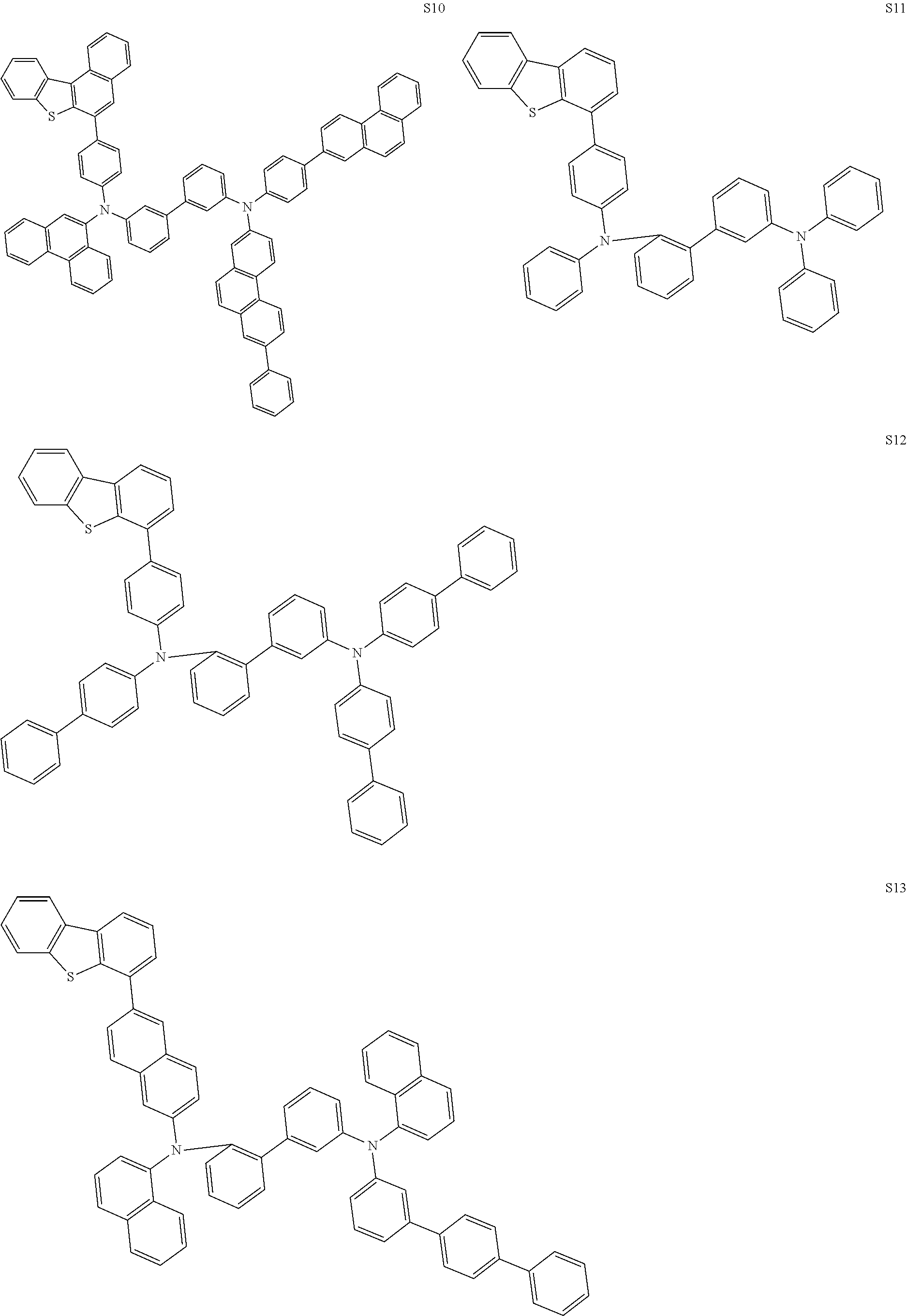 [SMILES: c1ccc(-c2ccc3c(ccc4cc(N(c5ccc(-c6ccc7c(ccc8ccccc87)c6)cc5)c5cccc(-c6cccc(N(c7ccc(-c8cc9ccccc9c9c8sc8ccccc89)cc7)c7cc8ccccc8c8ccccc78)c6)c5)ccc43)c2)cc1 ; c1ccc(N(c2ccccc2)c2cccc(-c3ccccc3N(c3ccccc3)c3ccc(-c4cccc5c4sc4ccccc45)cc3)c2)cc1 ; c1ccc(-c2ccc(N(c3ccc(-c4ccccc4)cc3)c3cccc(-c4ccccc4N(c4ccc(-c5ccccc5)cc4)c4ccc(-c5cccc6c5sc5ccccc56)cc4)c3)cc2)cc1 ; c1ccc(-c2ccc(-c3cccc(N(c4cccc(-c5ccccc5N(c5ccc6cc(-c7cccc8c7sc7ccccc78)ccc6c5)c5cccc6ccccc56)c4)c4cccc5ccccc45)c3)cc2)cc1]

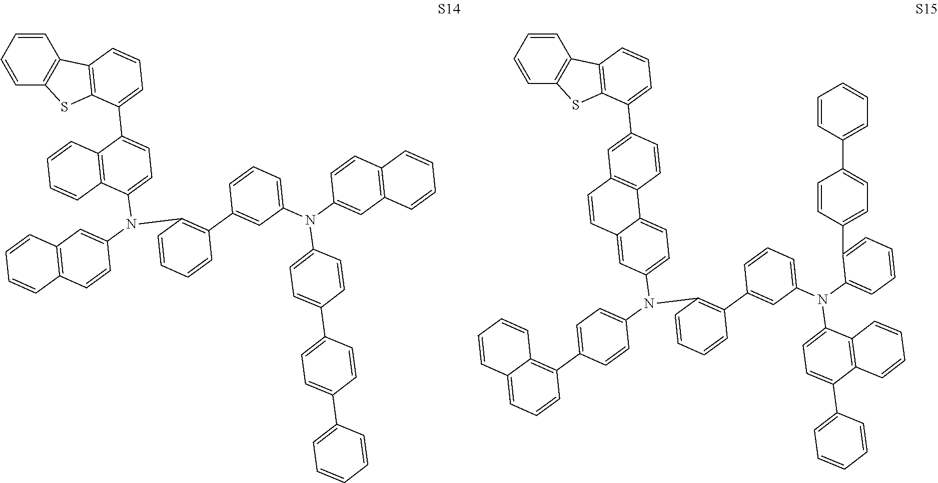 [SMILES: c1ccc(-c2ccc(-c3ccc(N(c4cccc(-c5ccccc5N(c5ccc6ccccc6c5)c5ccc(-c6cccc7c6sc6ccccc67)c6ccccc56)c4)c4ccc5ccccc5c4)cc3)cc2)cc1 ; c1ccc(-c2ccc(-c3ccccc3N(c3cccc(-c4ccccc4N(c4ccc(-c5cccc6ccccc56)cc4)c4ccc5c(ccc6cc(-c7cccc8c7sc7ccccc78)ccc65)c4)c3)c3ccc(-c4ccccc4)c4ccccc34)cc2)cc1]

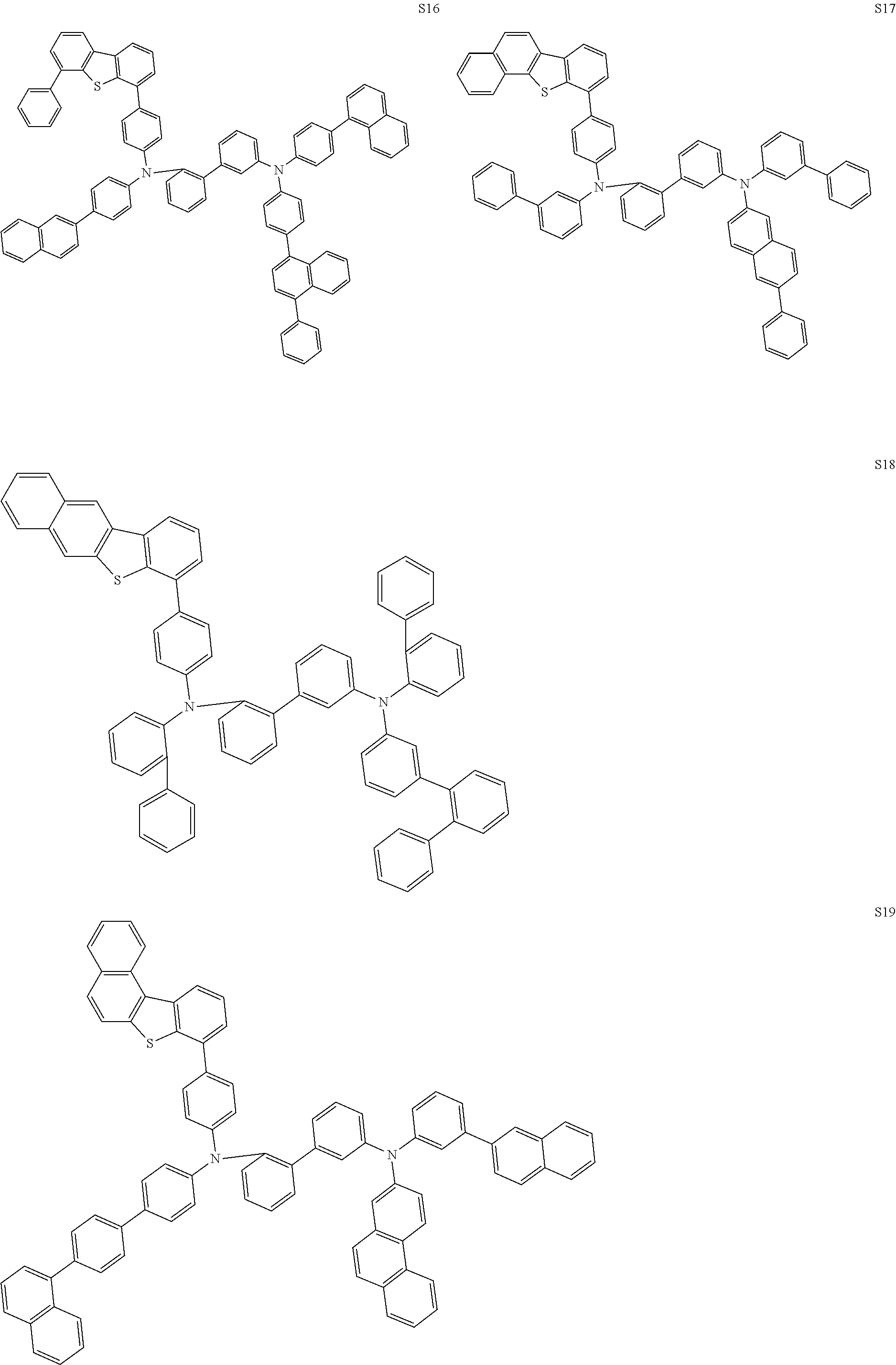

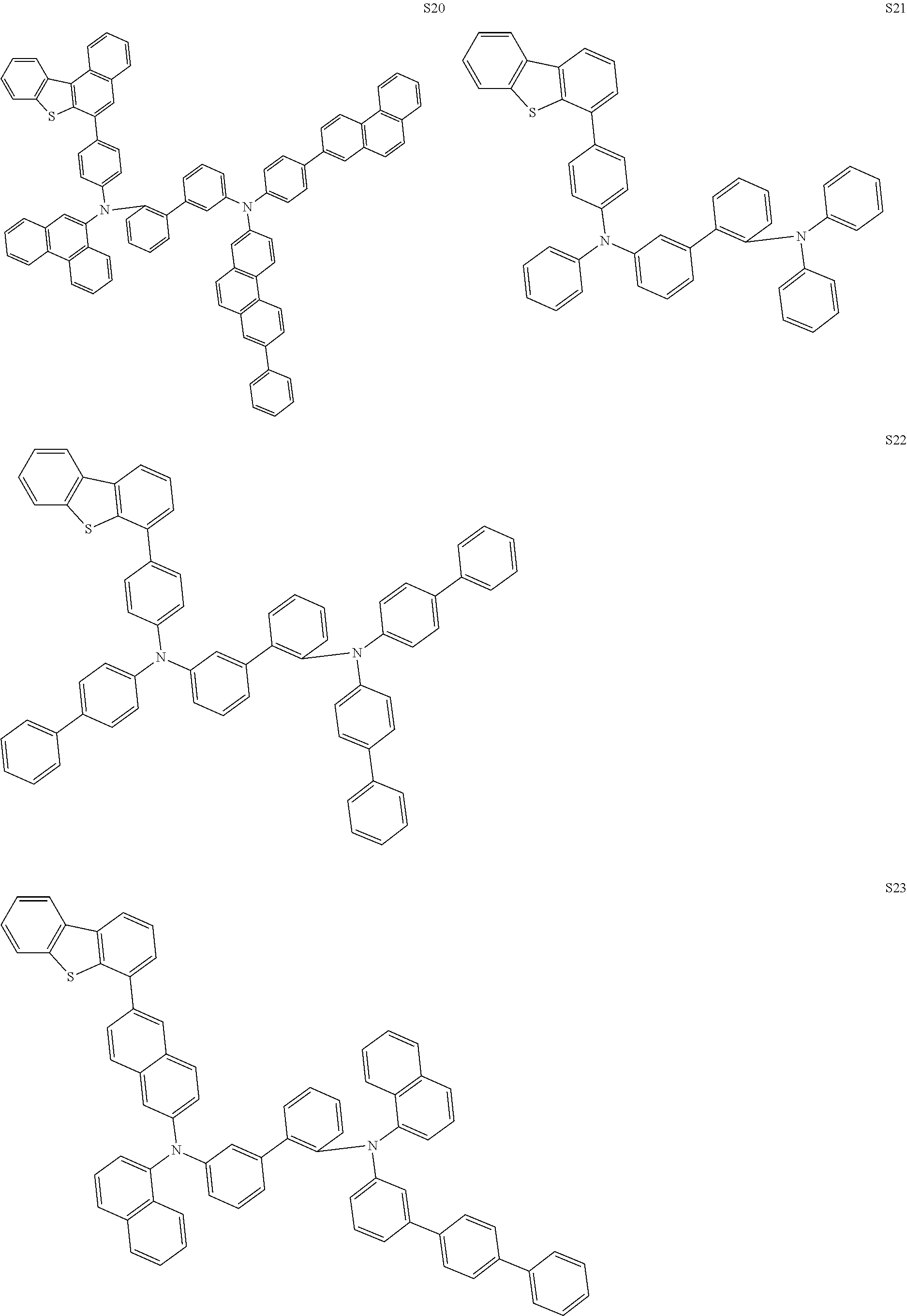 [SMILES: c1ccc(-c2ccc3c(ccc4cc(N(c5ccc(-c6ccc7c(ccc8ccccc87)c6)cc5)c5cccc(-c6ccccc6N(c6ccc(-c7cc8ccccc8c8c7sc7ccccc78)cc6)c6cc7ccccc7c7ccccc67)c5)ccc43)c2)cc1 ; c1ccc(N(c2ccc(-c3cccc4c3sc3ccccc34)cc2)c2cccc(-c3ccccc3N(c3ccccc3)c3ccccc3)c2)cc1 ; c1ccc(-c2ccc(N(c3ccc(-c4cccc5c4sc4ccccc45)cc3)c3cccc(-c4ccccc4N(c4ccc(-c5ccccc5)cc4)c4ccc(-c5ccccc5)cc4)c3)cc2)cc1 ; c1ccc(-c2ccc(-c3cccc(N(c4ccccc4-c4cccc(N(c5ccc6cc(-c7cccc8c7sc7ccccc78)ccc6c5)c5cccc6ccccc56)c4)c4cccc5ccccc45)c3)cc2)cc1]

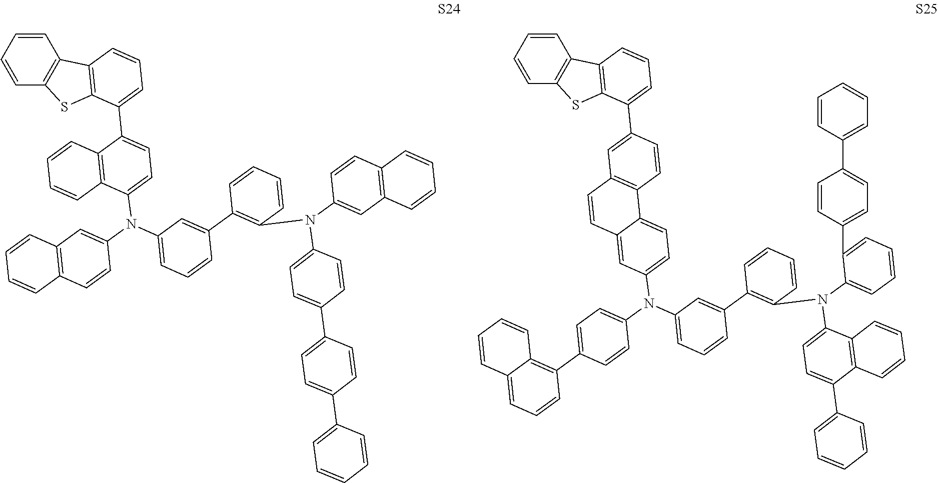 [SMILES: c1ccc(-c2ccc(-c3ccc(N(c4ccc5ccccc5c4)c4ccccc4-c4cccc(N(c5ccc6ccccc6c5)c5ccc(-c6cccc7c6sc6ccccc67)c6ccccc56)c4)cc3)cc2)cc1 ; c1ccc(-c2ccc(-c3ccccc3N(c3ccccc3-c3cccc(N(c4ccc(-c5cccc6ccccc56)cc4)c4ccc5c(ccc6cc(-c7cccc8c7sc7ccccc78)ccc65)c4)c3)c3ccc(-c4ccccc4)c4ccccc34)cc2)cc1]

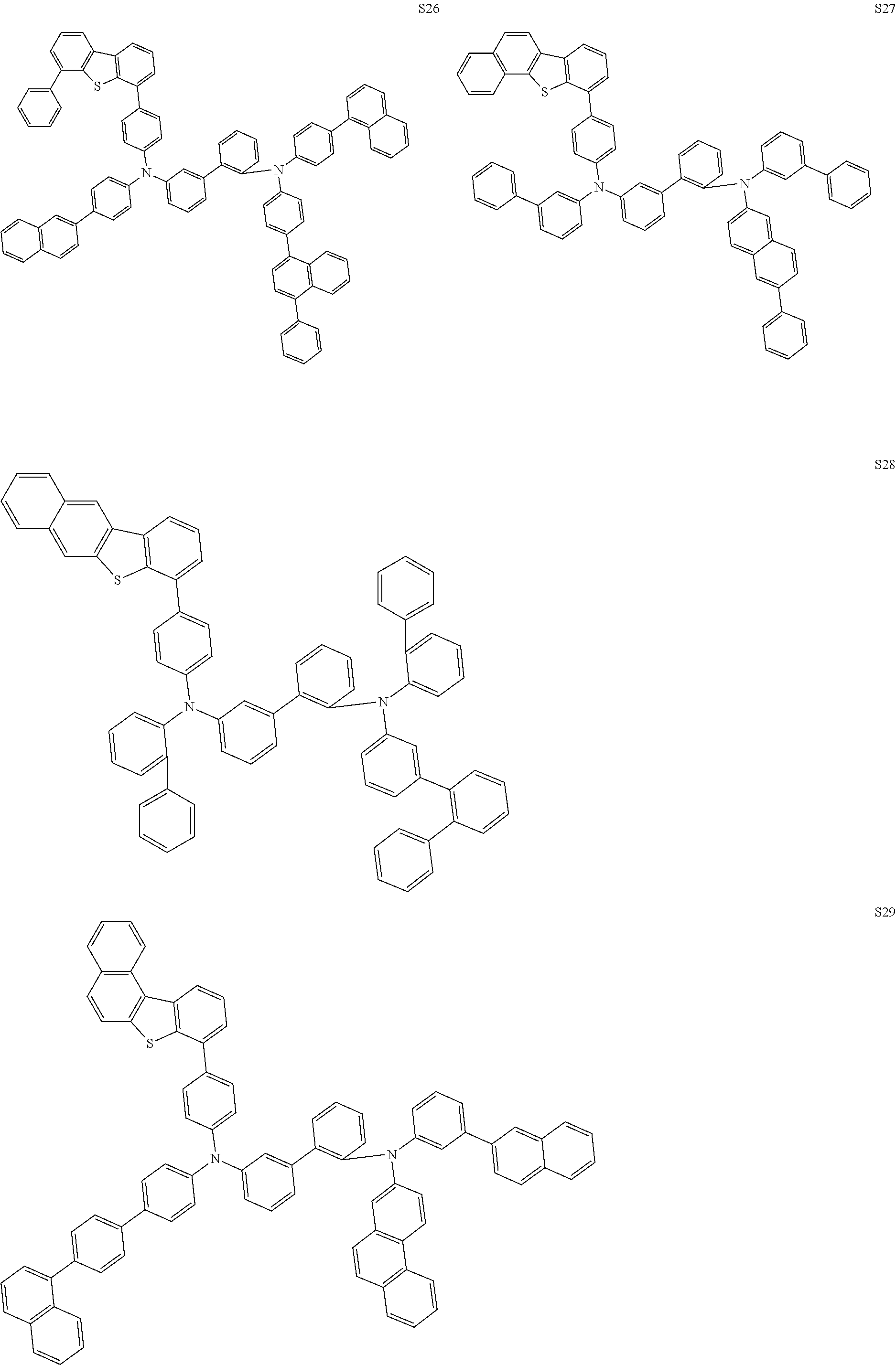

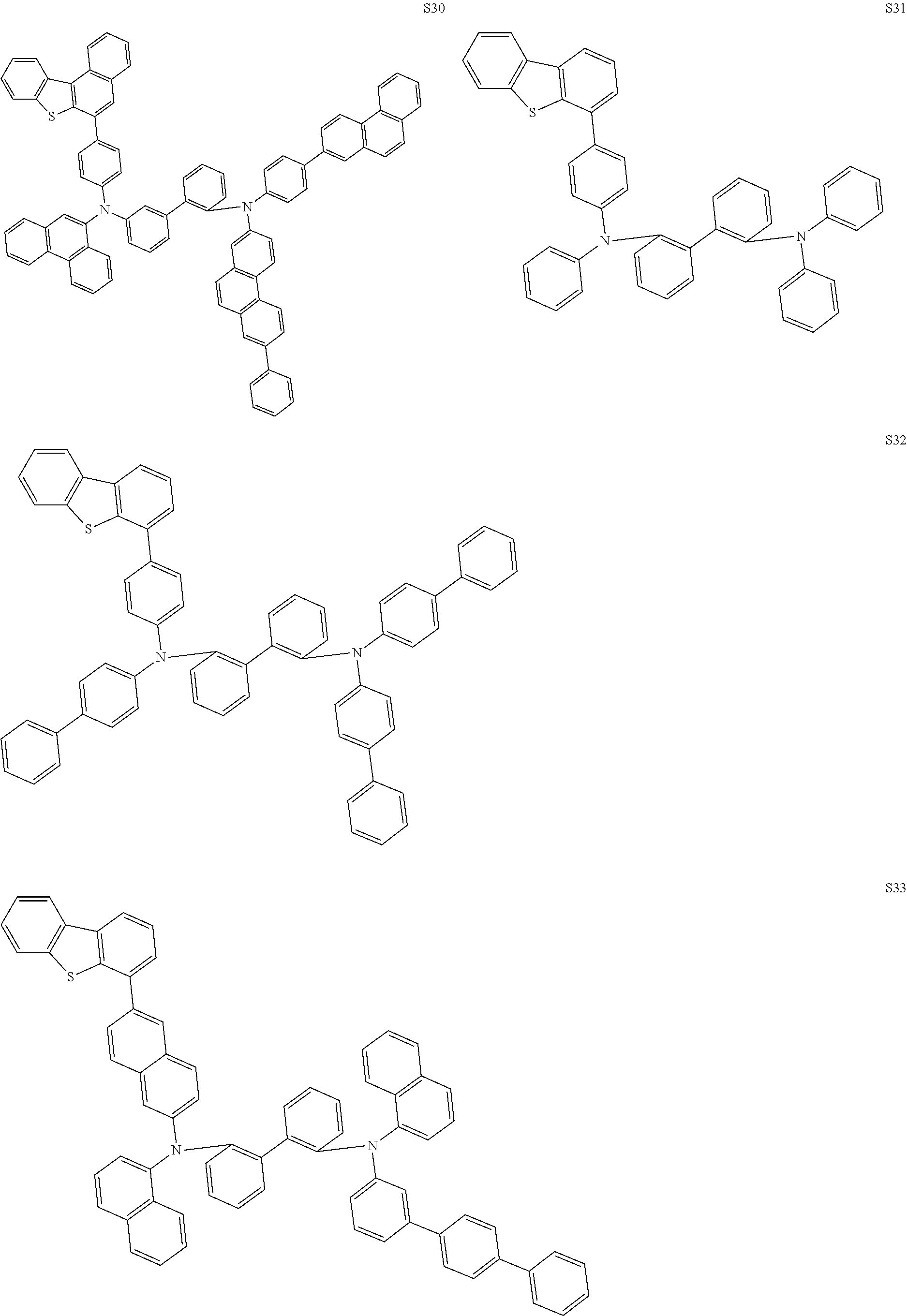 [SMILES: c1ccc(-c2ccc3c(ccc4cc(N(c5ccc(-c6ccc7c(ccc8ccccc87)c6)cc5)c5ccccc5-c5cccc(N(c6ccc(-c7cc8ccccc8c8c7sc7ccccc78)cc6)c6cc7ccccc7c7ccccc67)c5)ccc43)c2)cc1 ; c1ccc(N(c2ccccc2)c2ccccc2-c2ccccc2N(c2ccccc2)c2ccc(-c3cccc4c3sc3ccccc34)cc2)cc1 ; c1ccc(-c2ccc(N(c3ccc(-c4ccccc4)cc3)c3ccccc3-c3ccccc3N(c3ccc(-c4ccccc4)cc3)c3ccc(-c4cccc5c4sc4ccccc45)cc3)cc2)cc1 ; c1ccc(-c2ccc(-c3cccc(N(c4ccccc4-c4ccccc4N(c4ccc5cc(-c6cccc7c6sc6ccccc67)ccc5c4)c4cccc5ccccc45)c4cccc5ccccc45)c3)cc2)cc1]

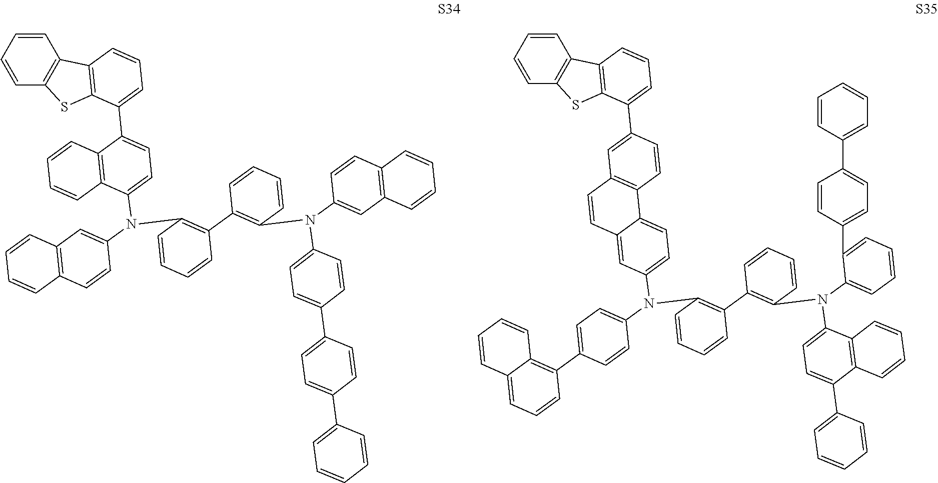 [SMILES: c1ccc(-c2ccc(-c3ccc(N(c4ccc5ccccc5c4)c4ccccc4-c4ccccc4N(c4ccc5ccccc5c4)c4ccc(-c5cccc6c5sc5ccccc56)c5ccccc45)cc3)cc2)cc1 ; c1ccc(-c2ccc(-c3ccccc3N(c3ccccc3-c3ccccc3N(c3ccc(-c4cccc5ccccc45)cc3)c3ccc4c(ccc5cc(-c6cccc7c6sc6ccccc67)ccc54)c3)c3ccc(-c4ccccc4)c4ccccc34)cc2)cc1]

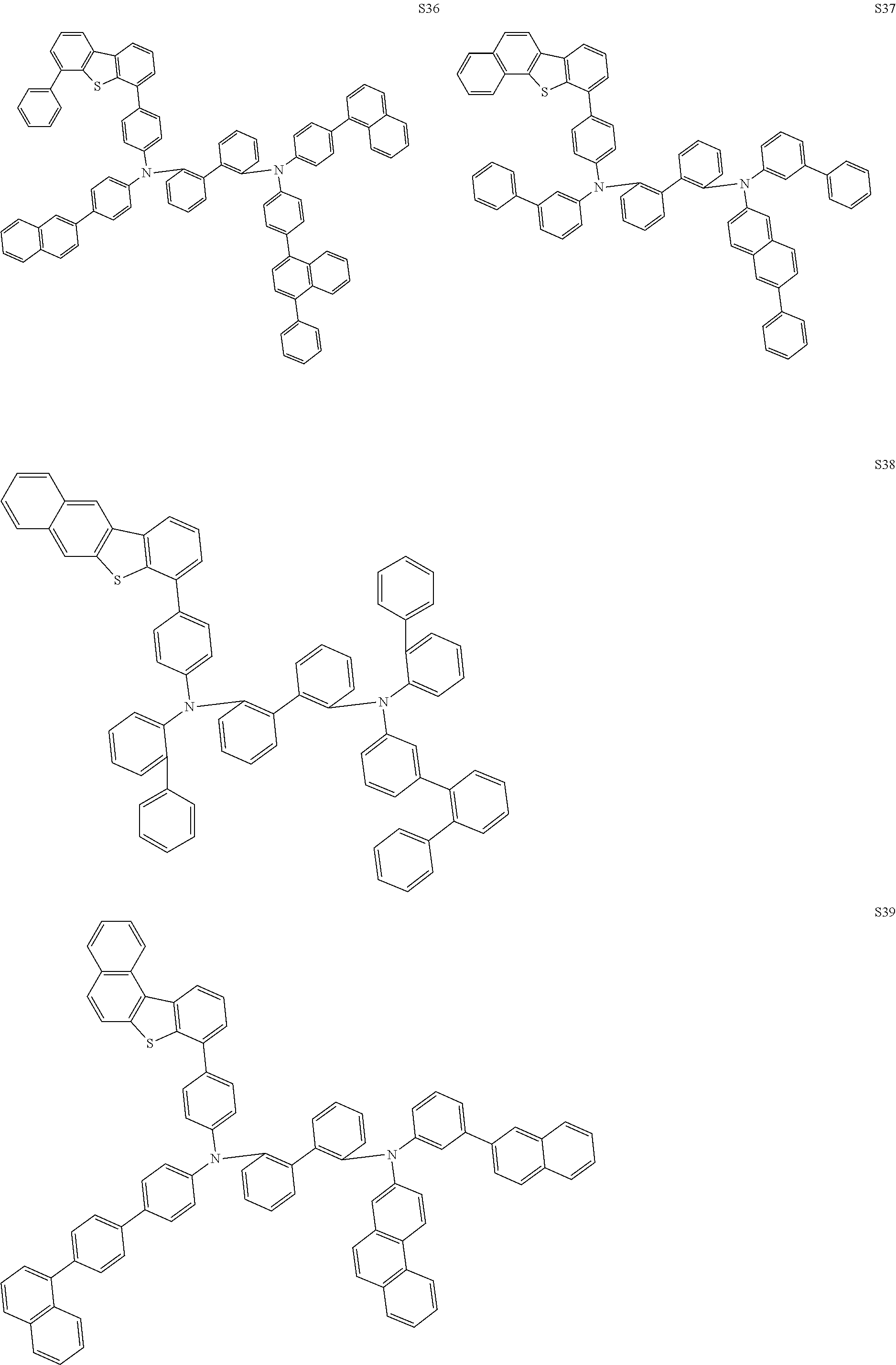

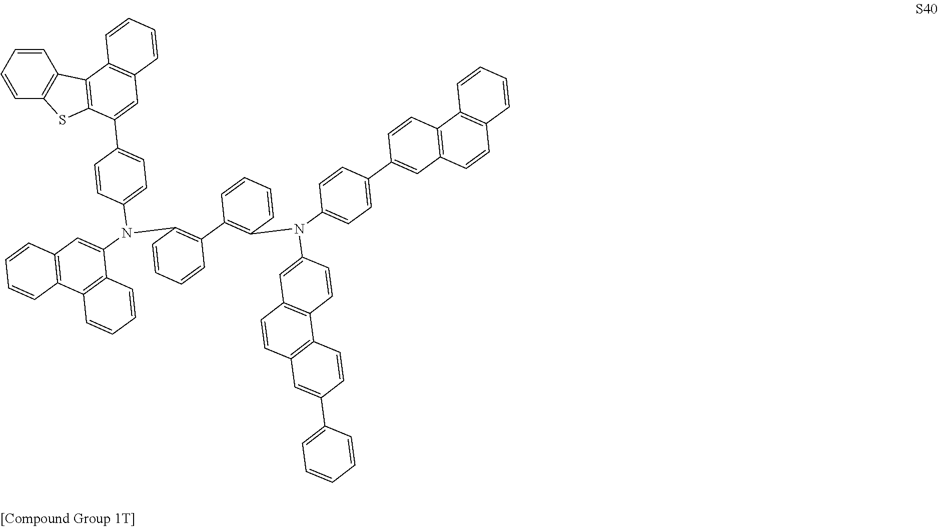 [SMILES: c1ccc(-c2ccc3c(ccc4cc(N(c5ccc(-c6ccc7c(ccc8ccccc87)c6)cc5)c5ccccc5-c5ccccc5N(c5ccc(-c6cc7ccccc7c7c6sc6ccccc67)cc5)c5cc6ccccc6c6ccccc56)ccc43)c2)cc1]

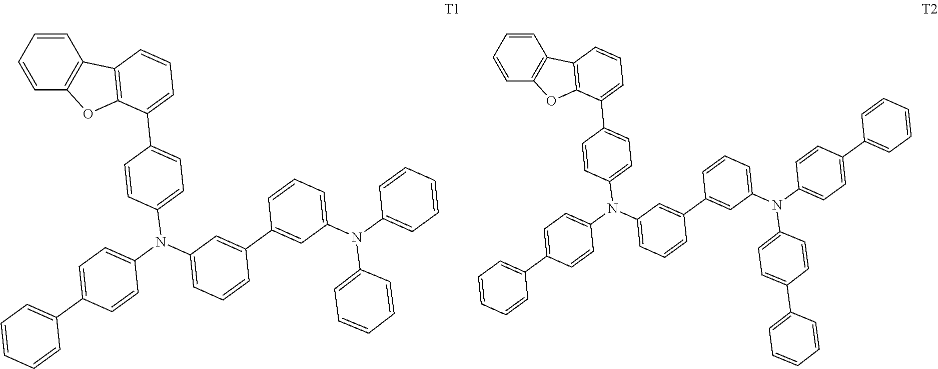 [SMILES: c1ccc(-c2ccc(N(c3ccc(-c4cccc5c4oc4ccccc45)cc3)c3cccc(-c4cccc(N(c5ccccc5)c5ccccc5)c4)c3)cc2)cc1 ; c1ccc(-c2ccc(N(c3ccc(-c4ccccc4)cc3)c3cccc(-c4cccc(N(c5ccc(-c6ccccc6)cc5)c5ccc(-c6cccc7c6oc6ccccc67)cc5)c4)c3)cc2)cc1]

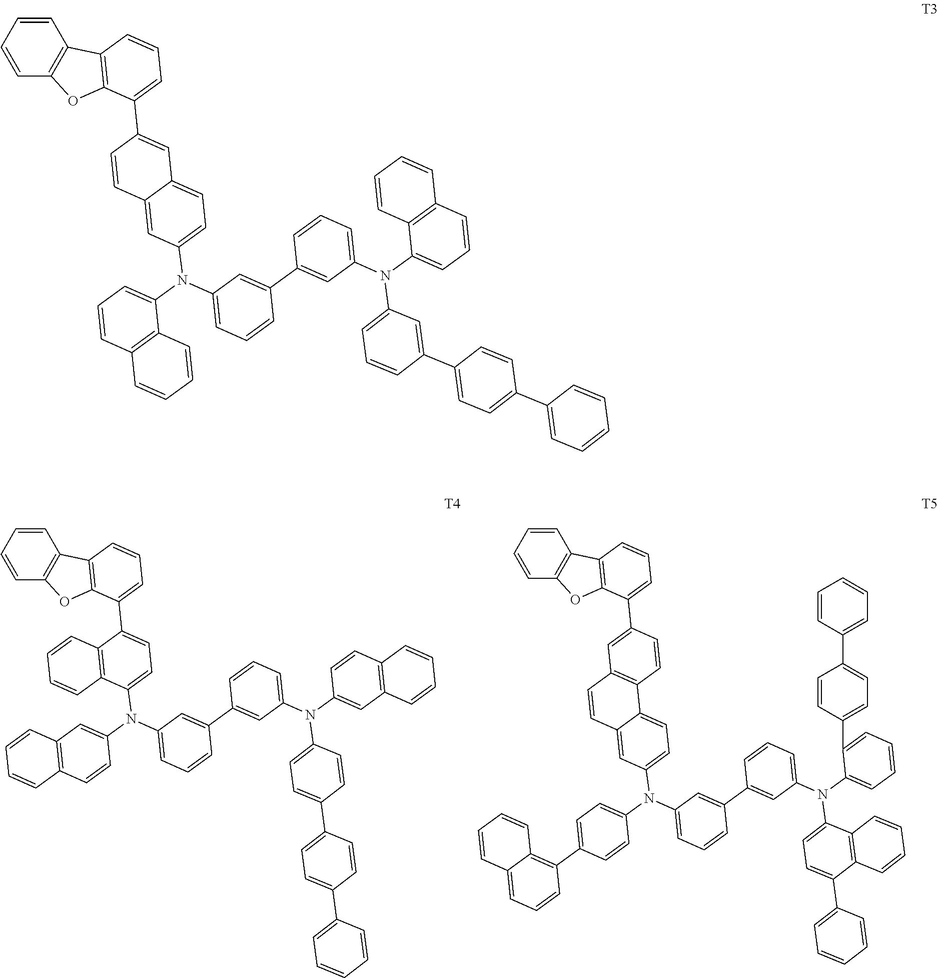 [SMILES: c1ccc(-c2ccc(-c3cccc(N(c4cccc(-c5cccc(N(c6ccc7cc(-c8cccc9c8oc8ccccc89)ccc7c6)c6cccc7ccccc67)c5)c4)c4cccc5ccccc45)c3)cc2)cc1 ; c1ccc(-c2ccc(-c3ccc(N(c4cccc(-c5cccc(N(c6ccc7ccccc7c6)c6ccc(-c7cccc8c7oc7ccccc78)c7ccccc67)c5)c4)c4ccc5ccccc5c4)cc3)cc2)cc1 ; c1ccc(-c2ccc(-c3ccccc3N(c3cccc(-c4cccc(N(c5ccc(-c6cccc7ccccc67)cc5)c5ccc6c(ccc7cc(-c8cccc9c8oc8ccccc89)ccc76)c5)c4)c3)c3ccc(-c4ccccc4)c4ccccc34)cc2)cc1]

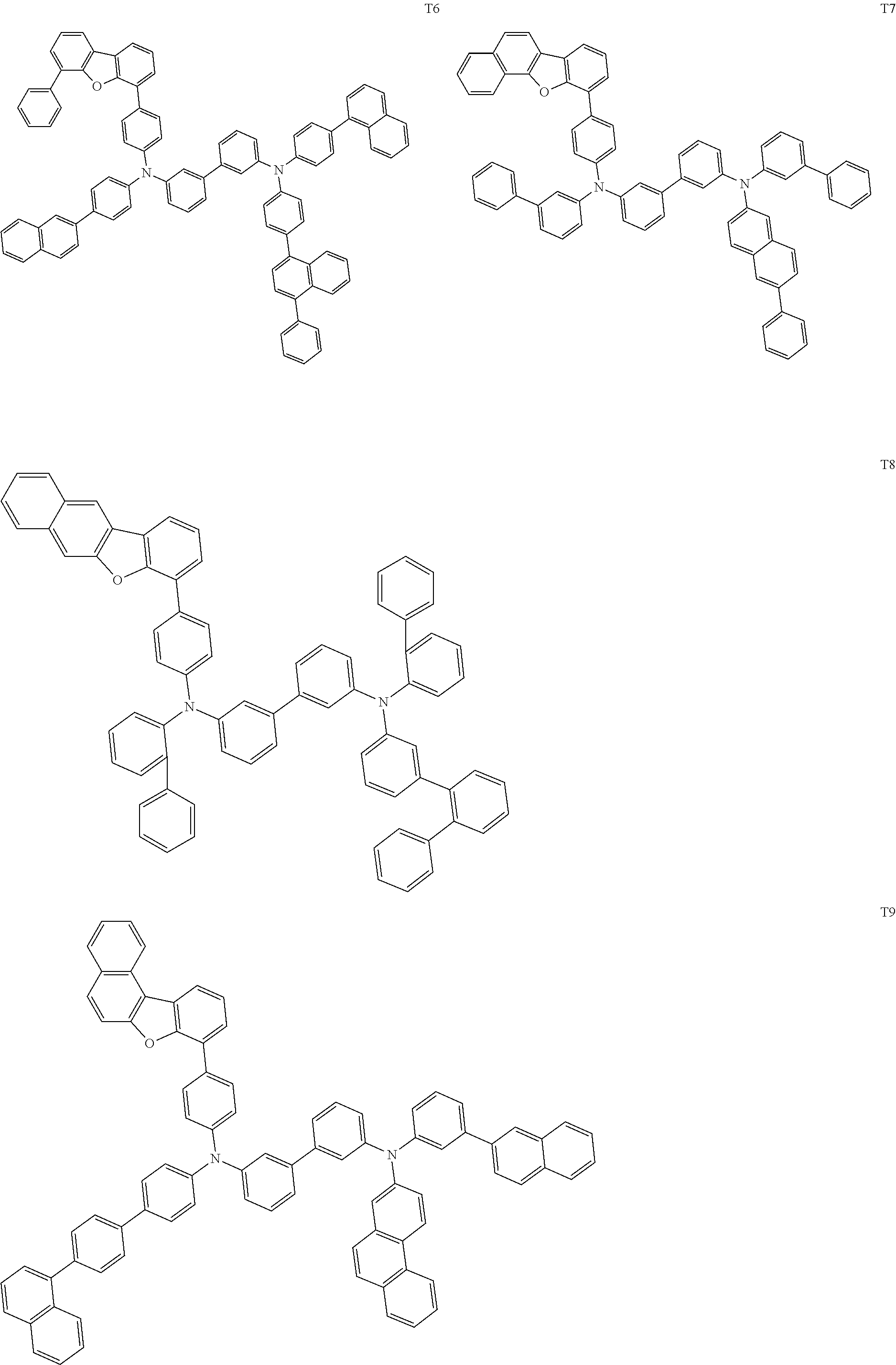

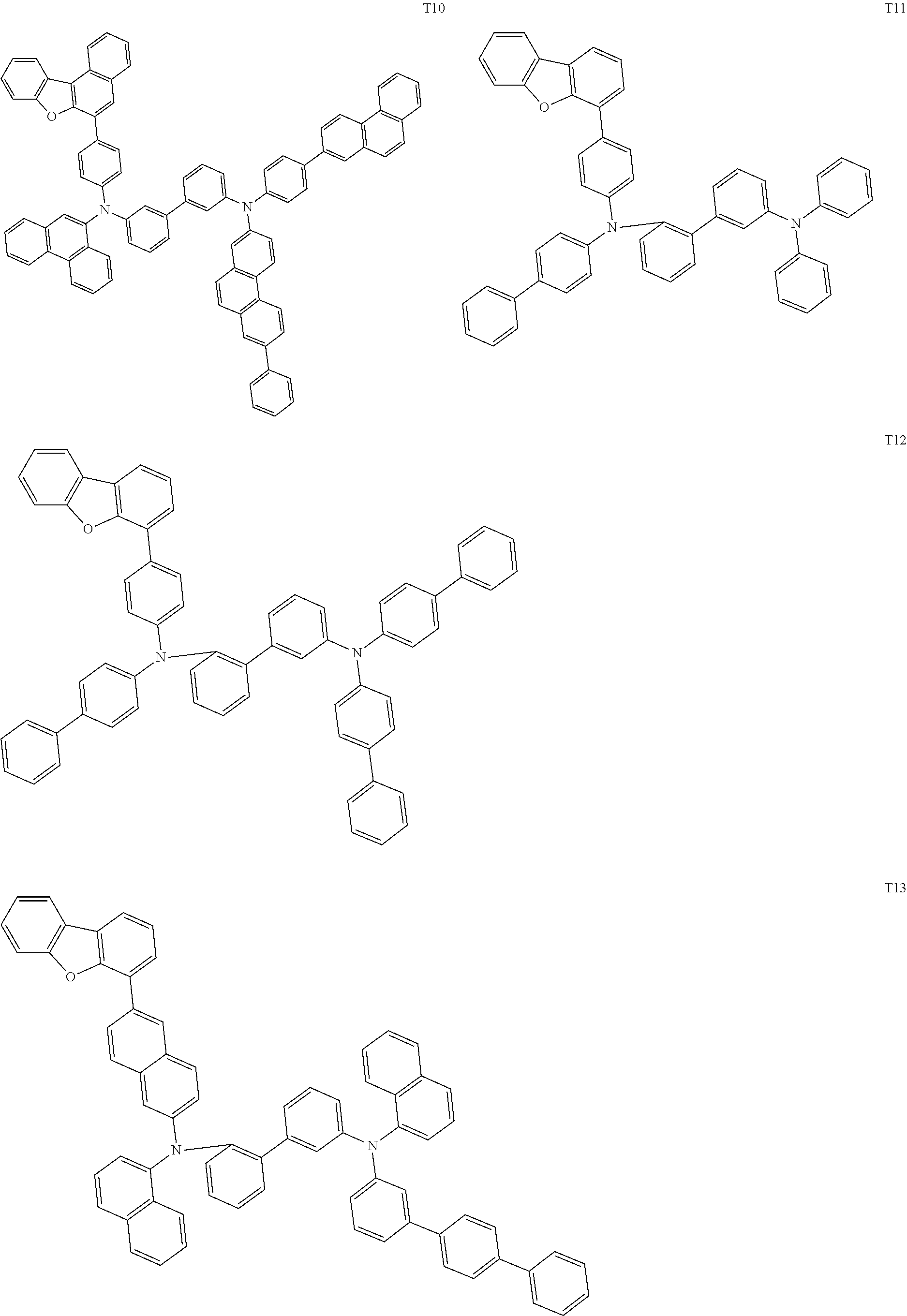 [SMILES: c1ccc(-c2ccc3c(ccc4cc(N(c5ccc(-c6ccc7c(ccc8ccccc87)c6)cc5)c5cccc(-c6cccc(N(c7ccc(-c8cc9ccccc9c9c8oc8ccccc89)cc7)c7cc8ccccc8c8ccccc78)c6)c5)ccc43)c2)cc1 ; c1ccc(-c2ccc(N(c3ccc(-c4cccc5c4oc4ccccc45)cc3)c3ccccc3-c3cccc(N(c4ccccc4)c4ccccc4)c3)cc2)cc1 ; c1ccc(-c2ccc(N(c3ccc(-c4ccccc4)cc3)c3cccc(-c4ccccc4N(c4ccc(-c5ccccc5)cc4)c4ccc(-c5cccc6c5oc5ccccc56)cc4)c3)cc2)cc1 ; c1ccc(-c2ccc(-c3cccc(N(c4cccc(-c5ccccc5N(c5ccc6cc(-c7cccc8c7oc7ccccc78)ccc6c5)c5cccc6ccccc56)c4)c4cccc5ccccc45)c3)cc2)cc1]

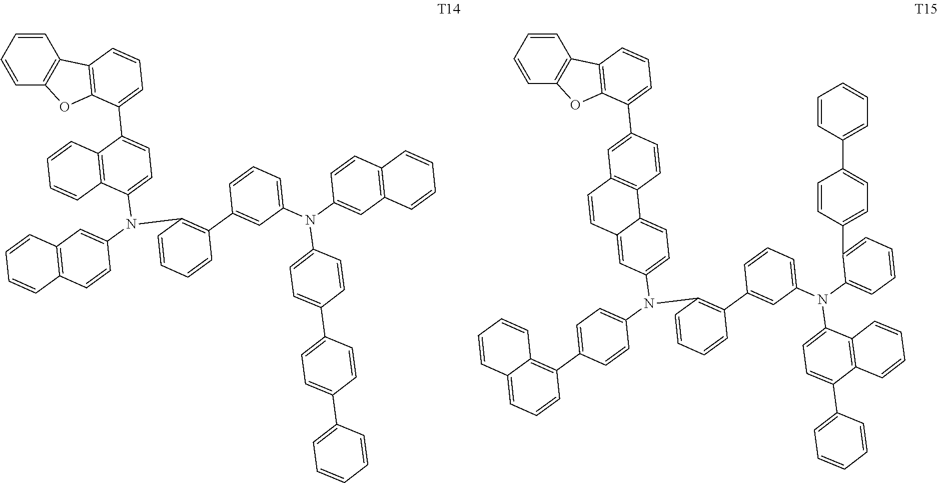 [SMILES: c1ccc(-c2ccc(-c3ccc(N(c4cccc(-c5ccccc5N(c5ccc6ccccc6c5)c5ccc(-c6cccc7c6oc6ccccc67)c6ccccc56)c4)c4ccc5ccccc5c4)cc3)cc2)cc1 ; c1ccc(-c2ccc(-c3ccccc3N(c3cccc(-c4ccccc4N(c4ccc(-c5cccc6ccccc56)cc4)c4ccc5c(ccc6cc(-c7cccc8c7oc7ccccc78)ccc65)c4)c3)c3ccc(-c4ccccc4)c4ccccc34)cc2)cc1]

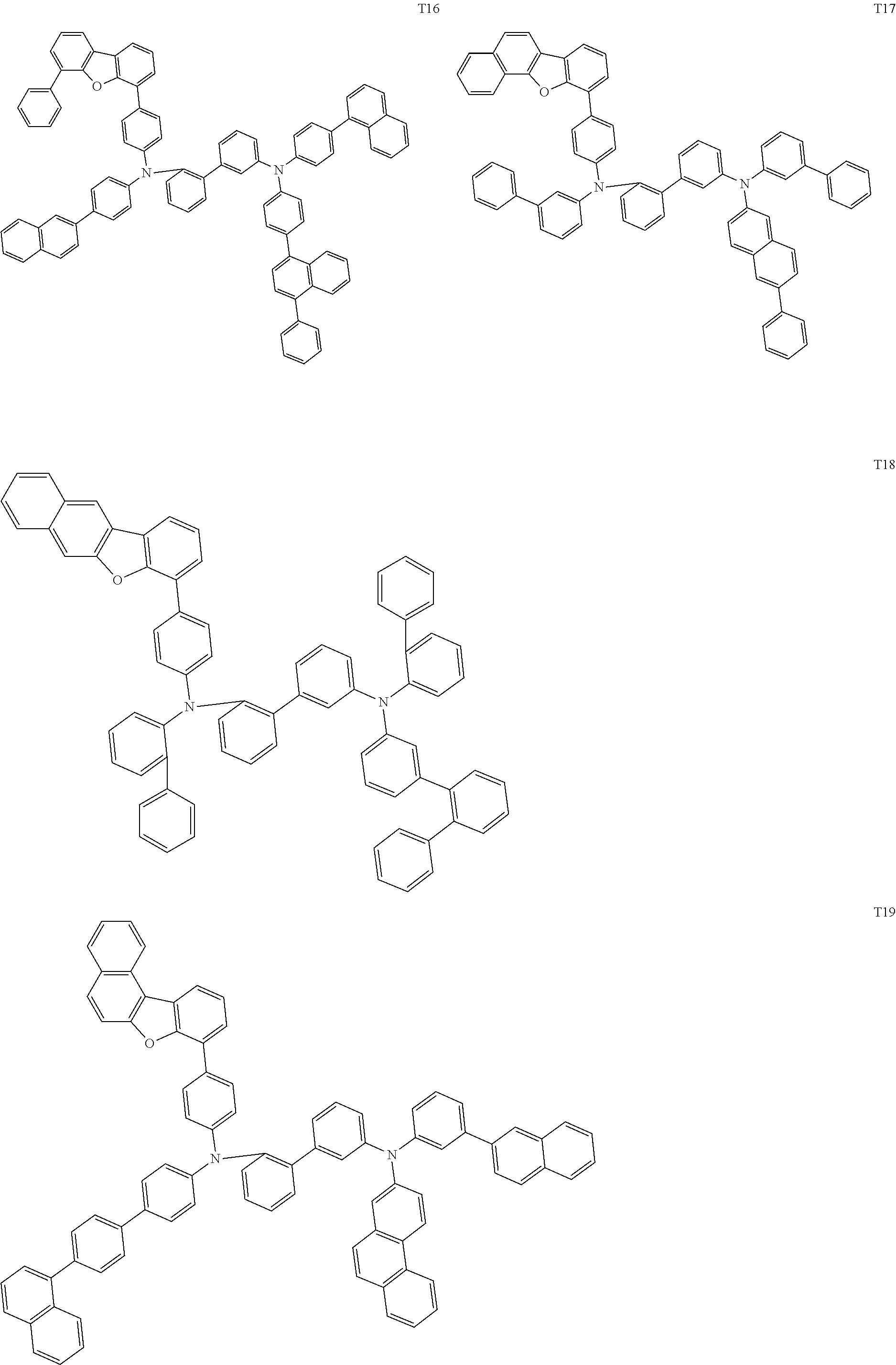

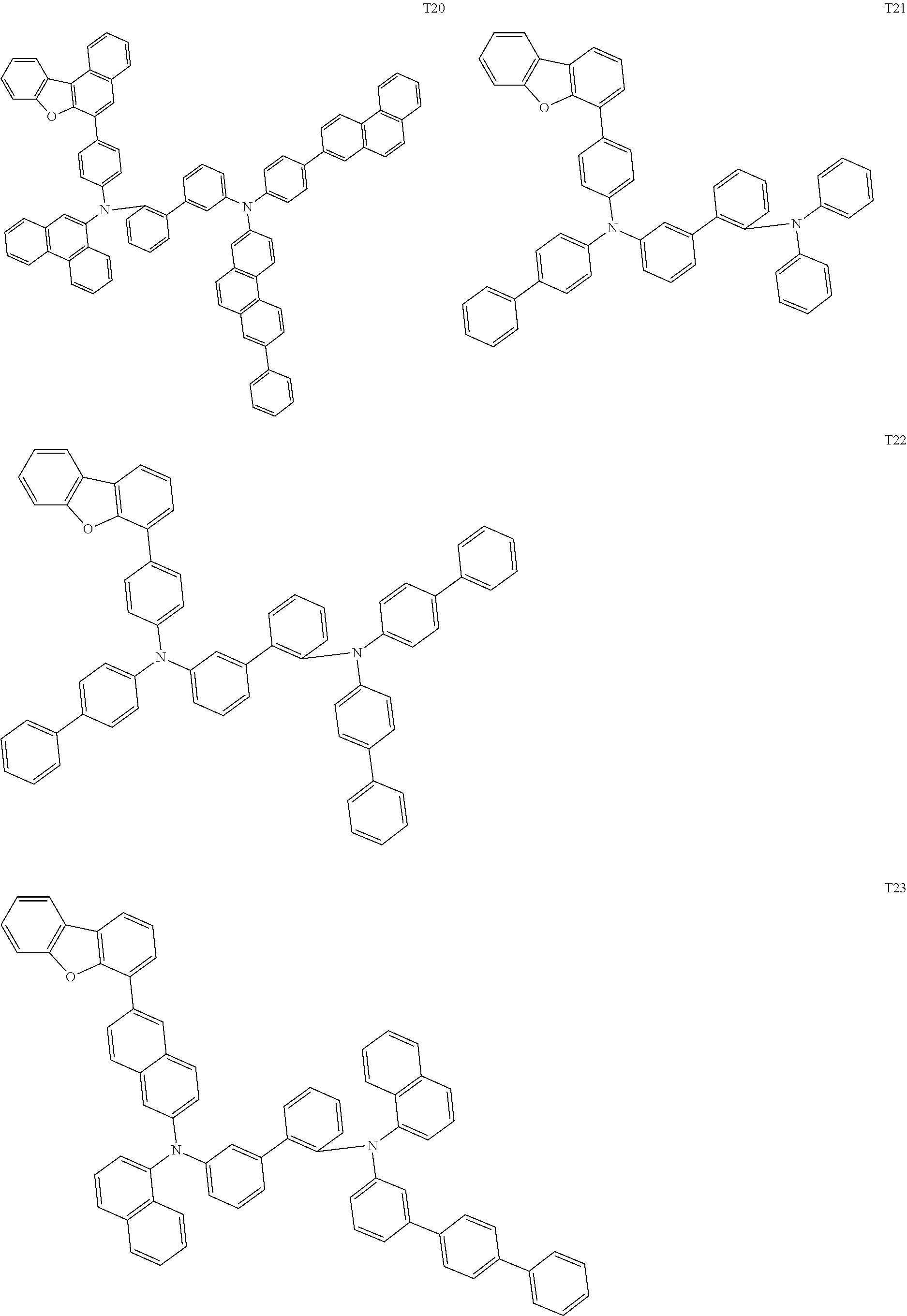 [SMILES: c1ccc(-c2ccc3c(ccc4cc(N(c5ccc(-c6ccc7c(ccc8ccccc87)c6)cc5)c5cccc(-c6ccccc6N(c6ccc(-c7cc8ccccc8c8c7oc7ccccc78)cc6)c6cc7ccccc7c7ccccc67)c5)ccc43)c2)cc1 ; c1ccc(-c2ccc(N(c3ccc(-c4cccc5c4oc4ccccc45)cc3)c3cccc(-c4ccccc4N(c4ccccc4)c4ccccc4)c3)cc2)cc1 ; c1ccc(-c2ccc(N(c3ccc(-c4cccc5c4oc4ccccc45)cc3)c3cccc(-c4ccccc4N(c4ccc(-c5ccccc5)cc4)c4ccc(-c5ccccc5)cc4)c3)cc2)cc1 ; c1ccc(-c2ccc(-c3cccc(N(c4ccccc4-c4cccc(N(c5ccc6cc(-c7cccc8c7oc7ccccc78)ccc6c5)c5cccc6ccccc56)c4)c4cccc5ccccc45)c3)cc2)cc1]

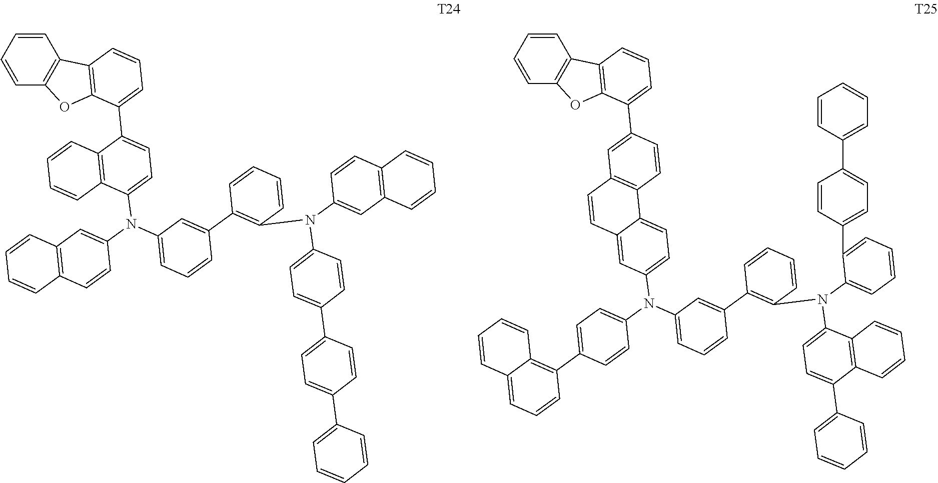 [SMILES: c1ccc(-c2ccc(-c3ccc(N(c4ccc5ccccc5c4)c4ccccc4-c4cccc(N(c5ccc6ccccc6c5)c5ccc(-c6cccc7c6oc6ccccc67)c6ccccc56)c4)cc3)cc2)cc1 ; c1ccc(-c2ccc(-c3ccccc3N(c3ccccc3-c3cccc(N(c4ccc(-c5cccc6ccccc56)cc4)c4ccc5c(ccc6cc(-c7cccc8c7oc7ccccc78)ccc65)c4)c3)c3ccc(-c4ccccc4)c4ccccc34)cc2)cc1]

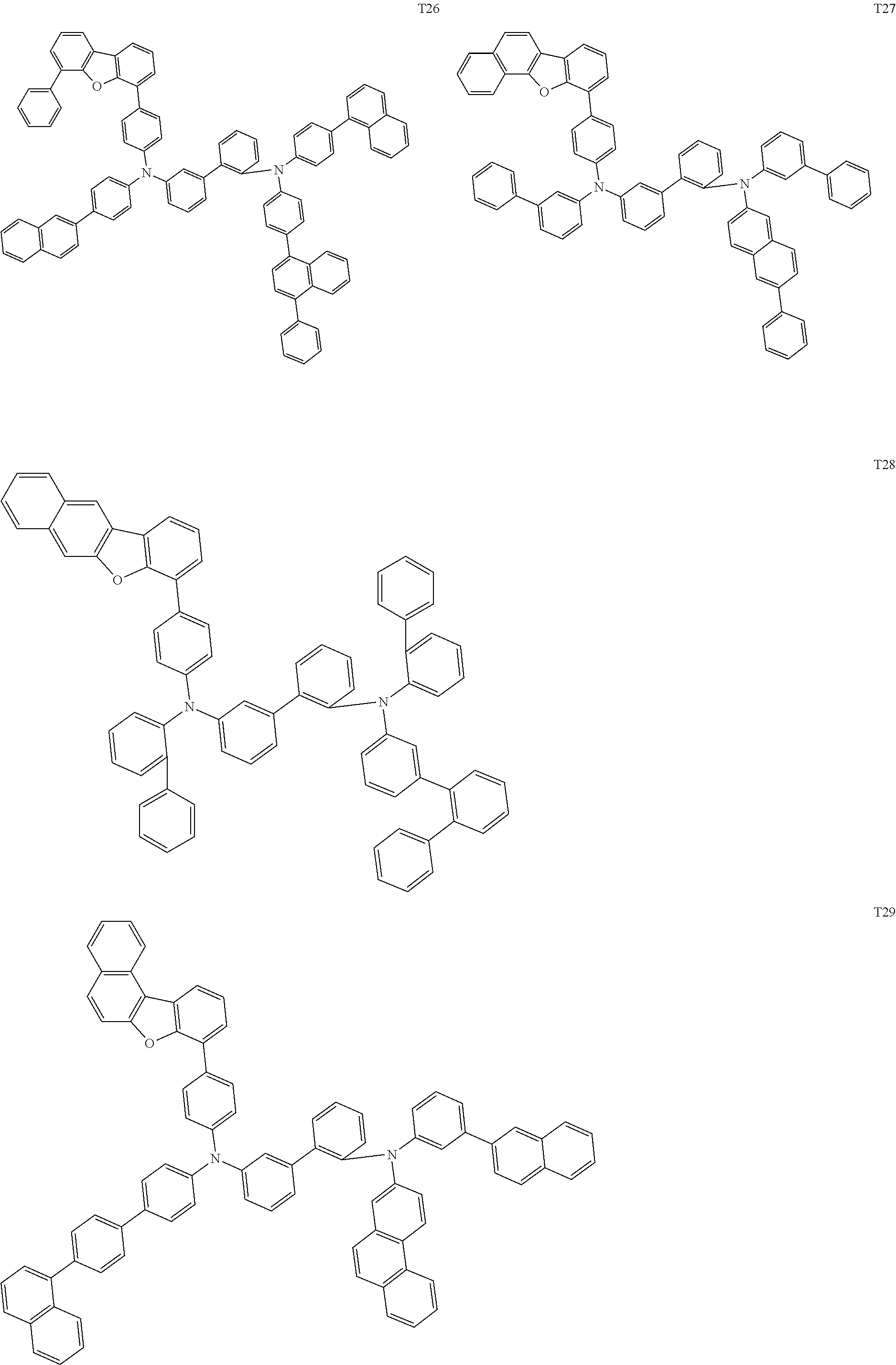

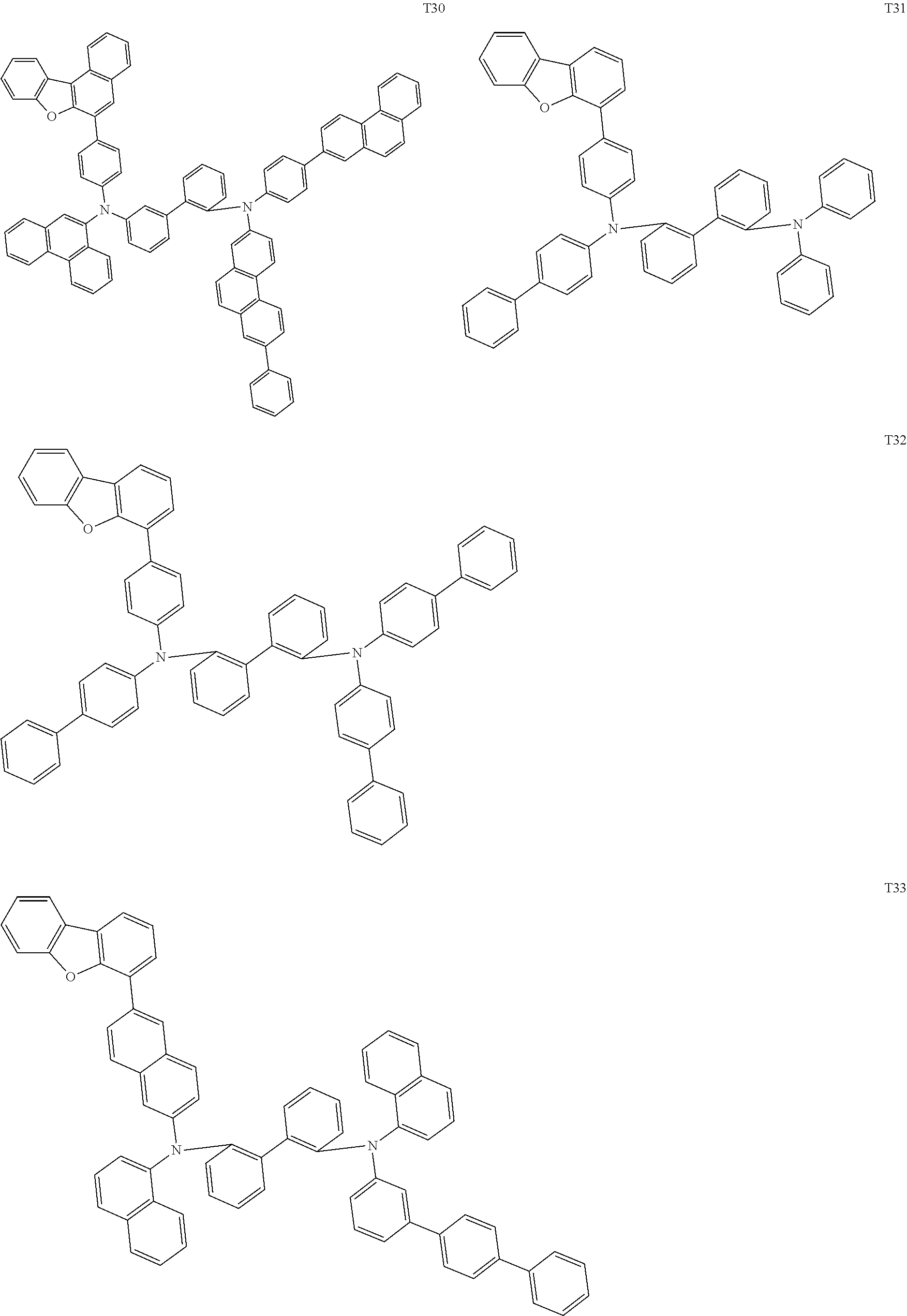 [SMILES: c1ccc(-c2ccc3c(ccc4cc(N(c5ccc(-c6ccc7c(ccc8ccccc87)c6)cc5)c5ccccc5-c5cccc(N(c6ccc(-c7cc8ccccc8c8c7oc7ccccc78)cc6)c6cc7ccccc7c7ccccc67)c5)ccc43)c2)cc1 ; c1ccc(-c2ccc(N(c3ccc(-c4cccc5c4oc4ccccc45)cc3)c3ccccc3-c3ccccc3N(c3ccccc3)c3ccccc3)cc2)cc1 ; c1ccc(-c2ccc(N(c3ccc(-c4ccccc4)cc3)c3ccccc3-c3ccccc3N(c3ccc(-c4ccccc4)cc3)c3ccc(-c4cccc5c4oc4ccccc45)cc3)cc2)cc1 ; c1ccc(-c2ccc(-c3cccc(N(c4ccccc4-c4ccccc4N(c4ccc5cc(-c6cccc7c6oc6ccccc67)ccc5c4)c4cccc5ccccc45)c4cccc5ccccc45)c3)cc2)cc1]

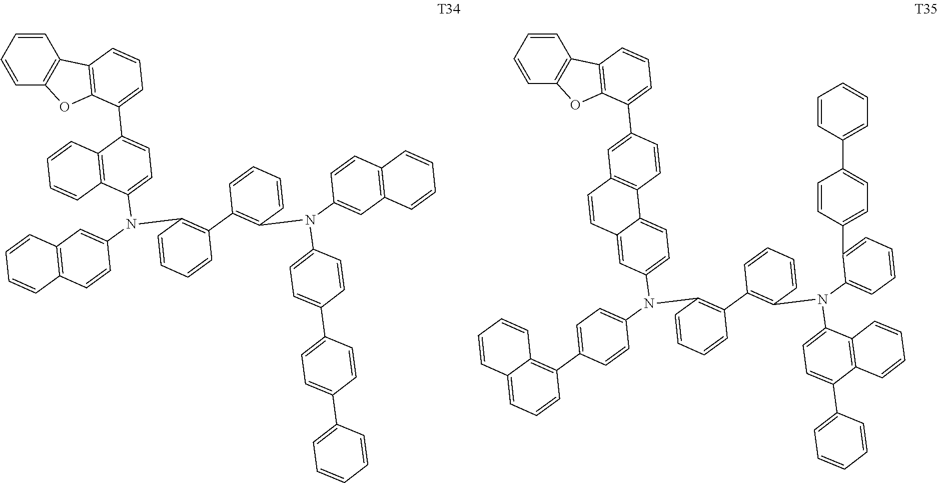 [SMILES: c1ccc(-c2ccc(-c3ccc(N(c4ccc5ccccc5c4)c4ccccc4-c4ccccc4N(c4ccc5ccccc5c4)c4ccc(-c5cccc6c5oc5ccccc56)c5ccccc45)cc3)cc2)cc1 ; c1ccc(-c2ccc(-c3ccccc3N(c3ccccc3-c3ccccc3N(c3ccc(-c4cccc5ccccc45)cc3)c3ccc4c(ccc5cc(-c6cccc7c6oc6ccccc67)ccc54)c3)c3ccc(-c4ccccc4)c4ccccc34)cc2)cc1]

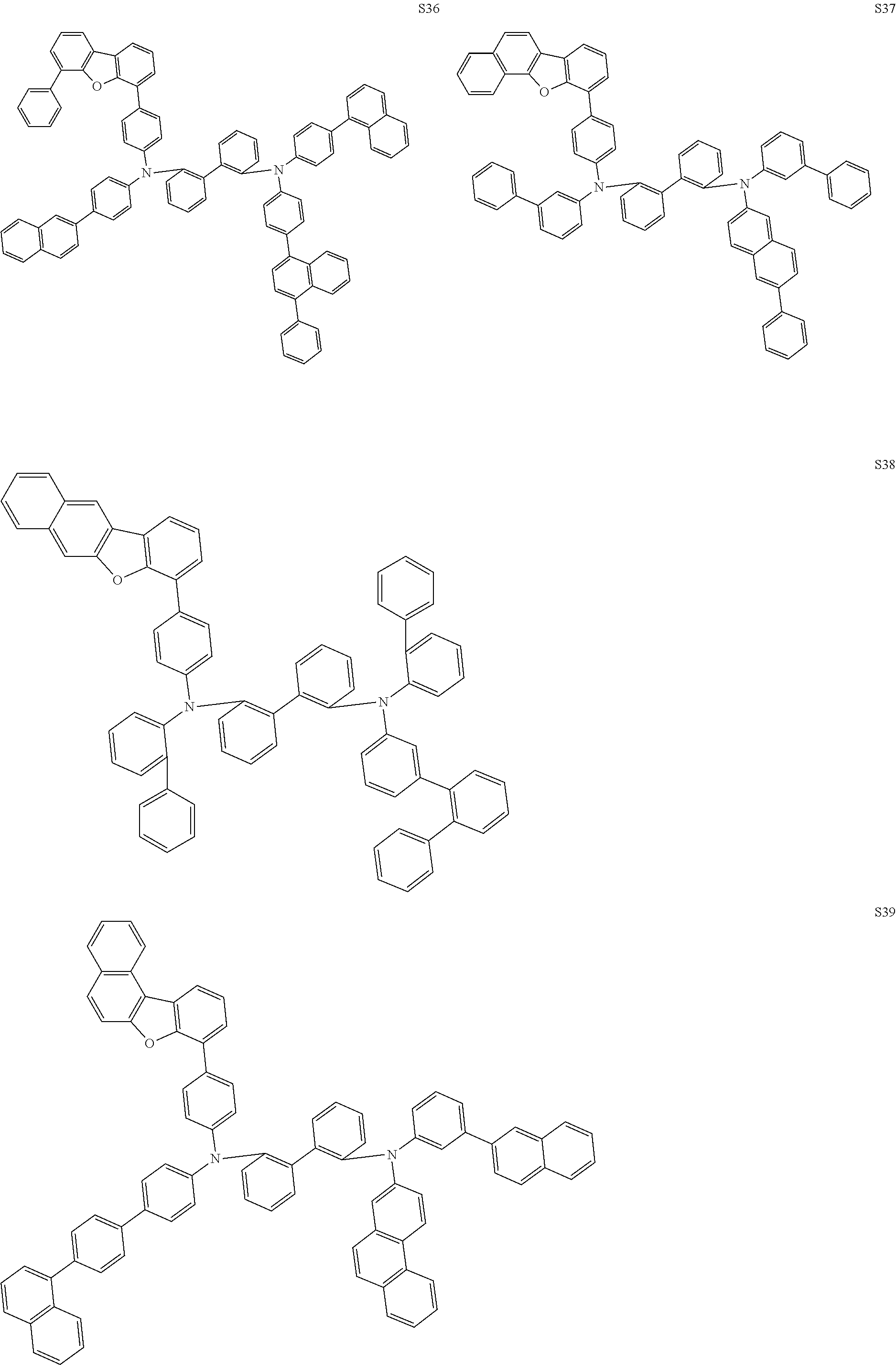

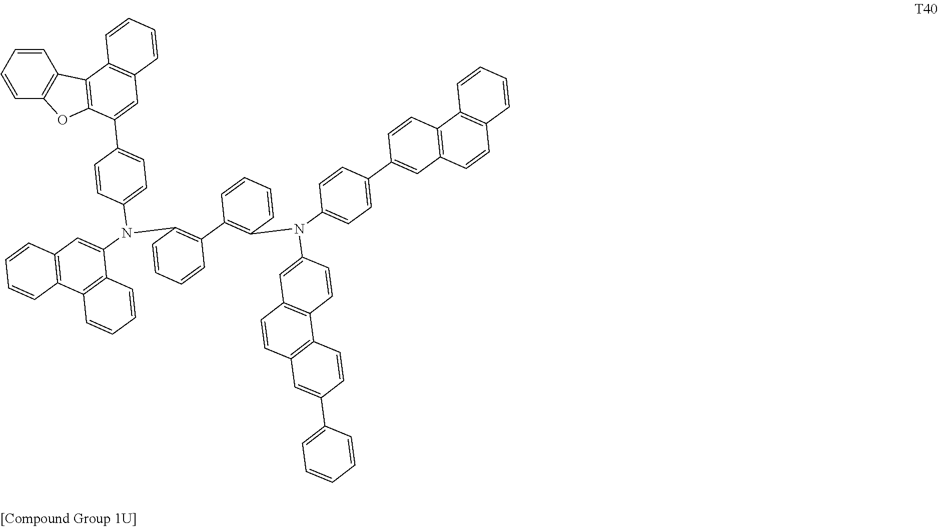 [SMILES: c1ccc(-c2ccc3c(ccc4cc(N(c5ccc(-c6ccc7c(ccc8ccccc87)c6)cc5)c5ccccc5-c5ccccc5N(c5ccc(-c6cc7ccccc7c7c6oc6ccccc67)cc5)c5cc6ccccc6c6ccccc56)ccc43)c2)cc1]

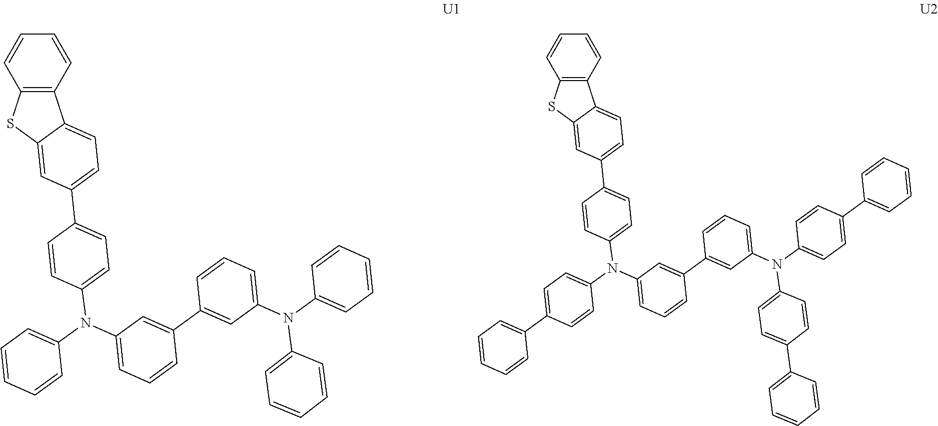 [SMILES: c1ccc(N(c2ccccc2)c2cccc(-c3cccc(N(c4ccccc4)c4ccc(-c5ccc6c(c5)sc5ccccc56)cc4)c3)c2)cc1 ; c1ccc(-c2ccc(N(c3ccc(-c4ccccc4)cc3)c3cccc(-c4cccc(N(c5ccc(-c6ccccc6)cc5)c5ccc(-c6ccc7c(c6)sc6ccccc67)cc5)c4)c3)cc2)cc1]

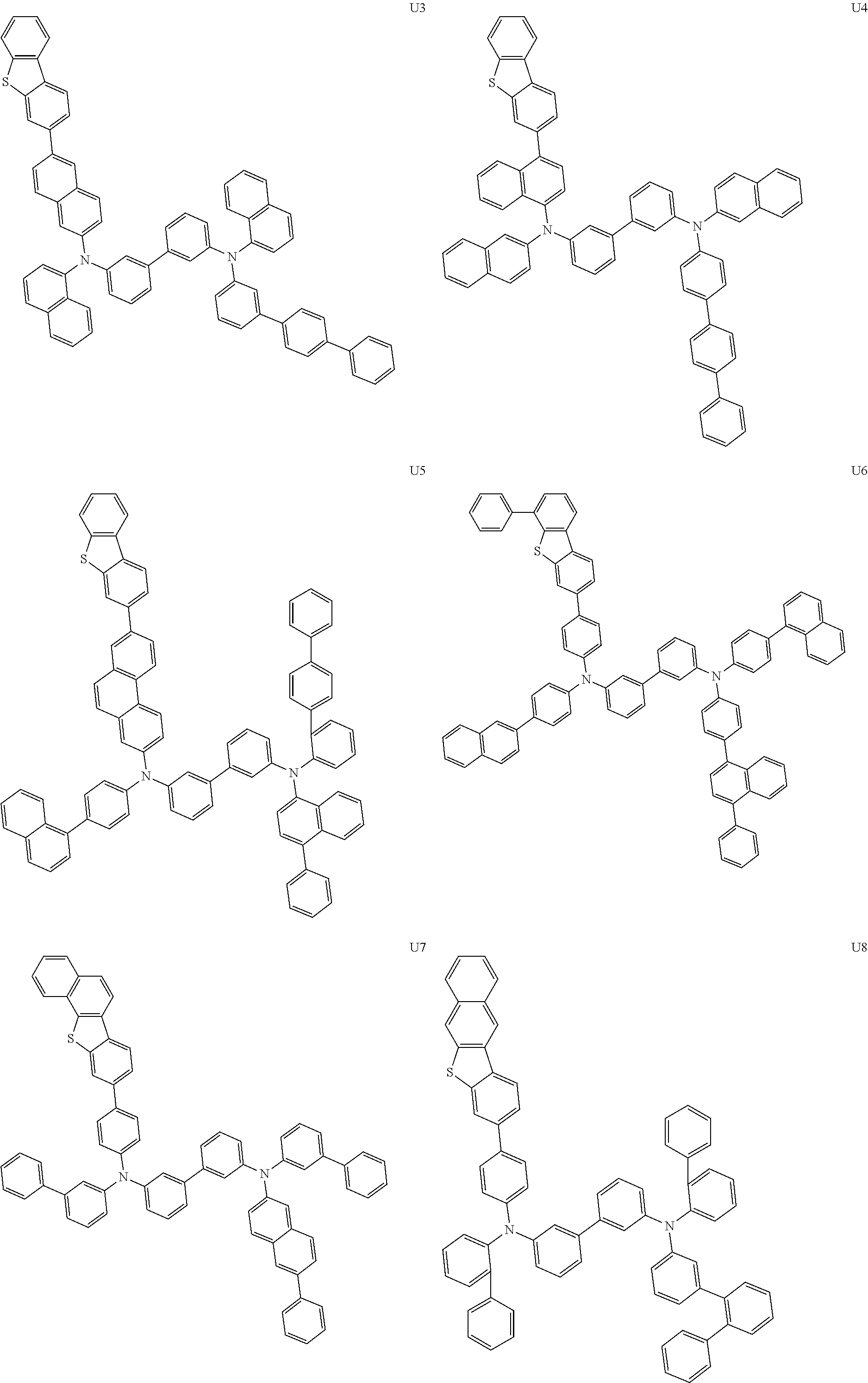

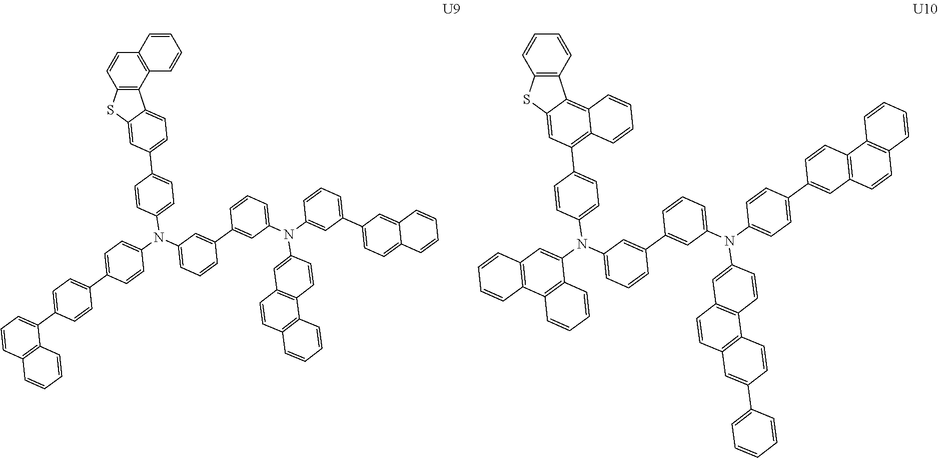 [SMILES: c1cc(-c2cccc(N(c3cccc(-c4ccc5ccccc5c4)c3)c3ccc4c(ccc5ccccc54)c3)c2)cc(N(c2ccc(-c3ccc(-c4cccc5ccccc45)cc3)cc2)c2ccc(-c3ccc4c(c3)sc3ccc5ccccc5c34)cc2)c1 ; c1ccc(-c2ccc3c(ccc4cc(N(c5ccc(-c6ccc7c(ccc8ccccc87)c6)cc5)c5cccc(-c6cccc(N(c7ccc(-c8cc9sc%10ccccc%10c9c9ccccc89)cc7)c7cc8ccccc8c8ccccc78)c6)c5)ccc43)c2)cc1]

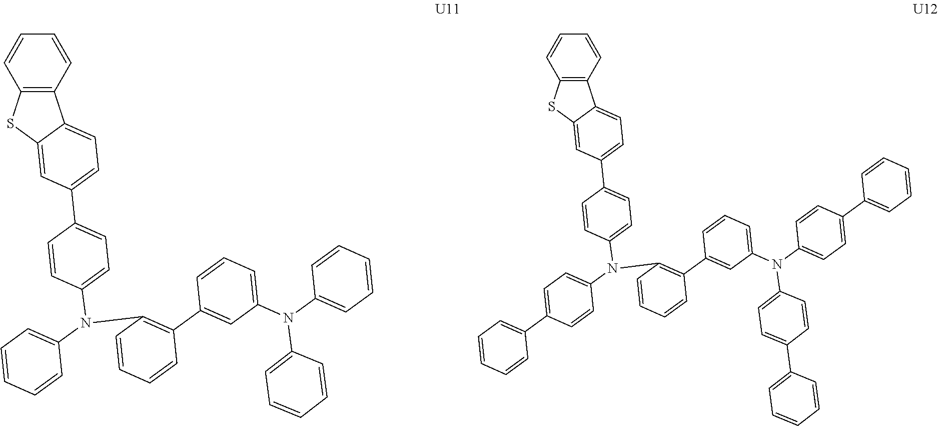 [SMILES: c1ccc(N(c2ccccc2)c2cccc(-c3ccccc3N(c3ccccc3)c3ccc(-c4ccc5c(c4)sc4ccccc45)cc3)c2)cc1 ; c1ccc(-c2ccc(N(c3ccc(-c4ccccc4)cc3)c3cccc(-c4ccccc4N(c4ccc(-c5ccccc5)cc4)c4ccc(-c5ccc6c(c5)sc5ccccc56)cc4)c3)cc2)cc1]

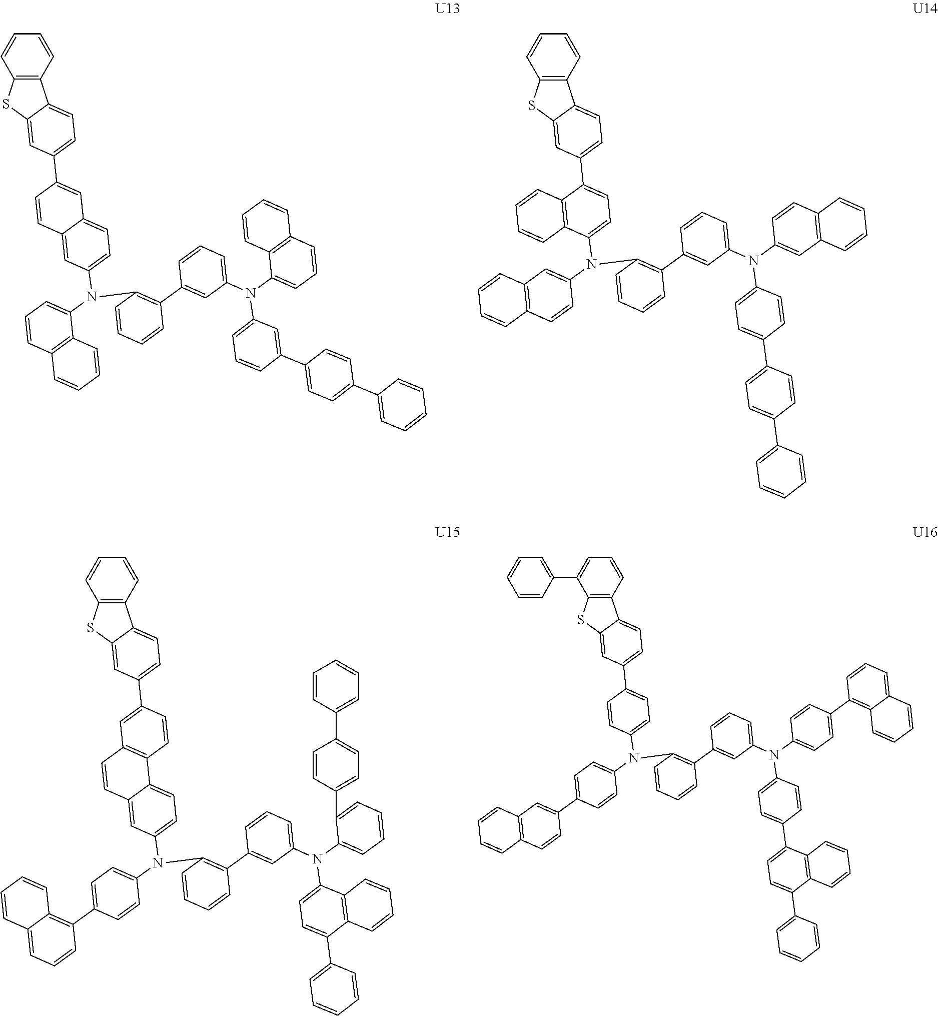

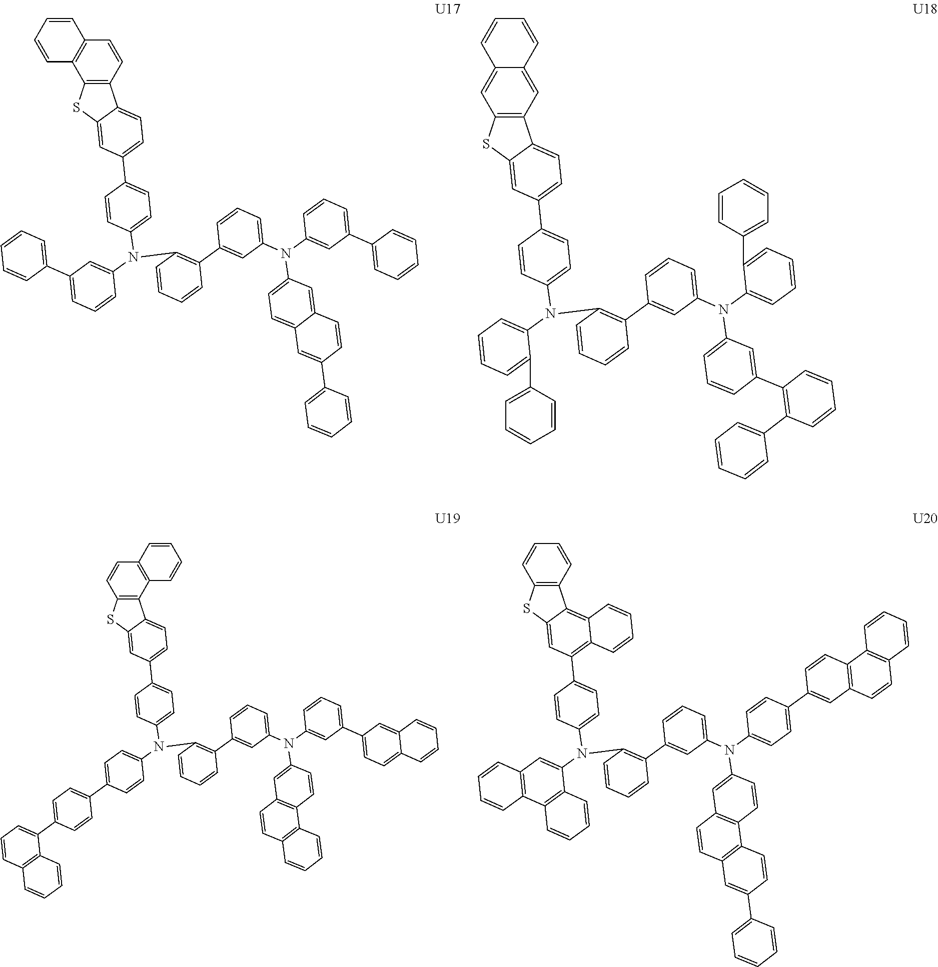

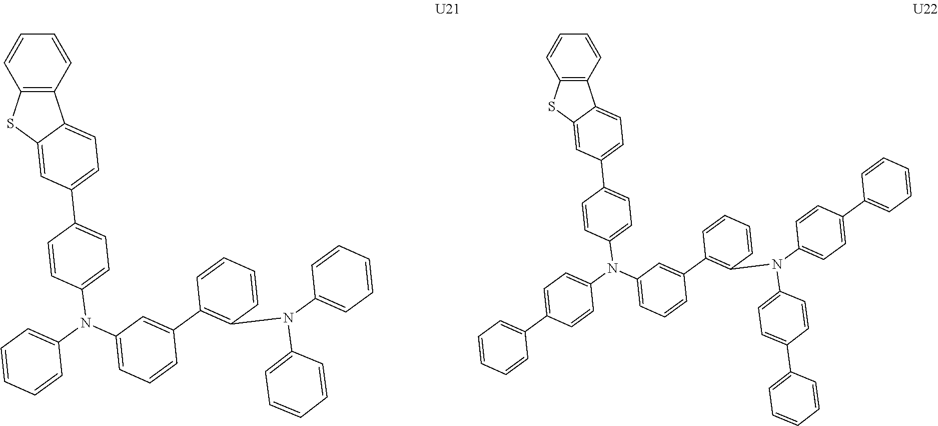 [SMILES: c1ccc(N(c2ccc(-c3ccc4c(c3)sc3ccccc34)cc2)c2cccc(-c3ccccc3N(c3ccccc3)c3ccccc3)c2)cc1 ; c1ccc(-c2ccc(N(c3ccc(-c4ccc5c(c4)sc4ccccc45)cc3)c3cccc(-c4ccccc4N(c4ccc(-c5ccccc5)cc4)c4ccc(-c5ccccc5)cc4)c3)cc2)cc1]

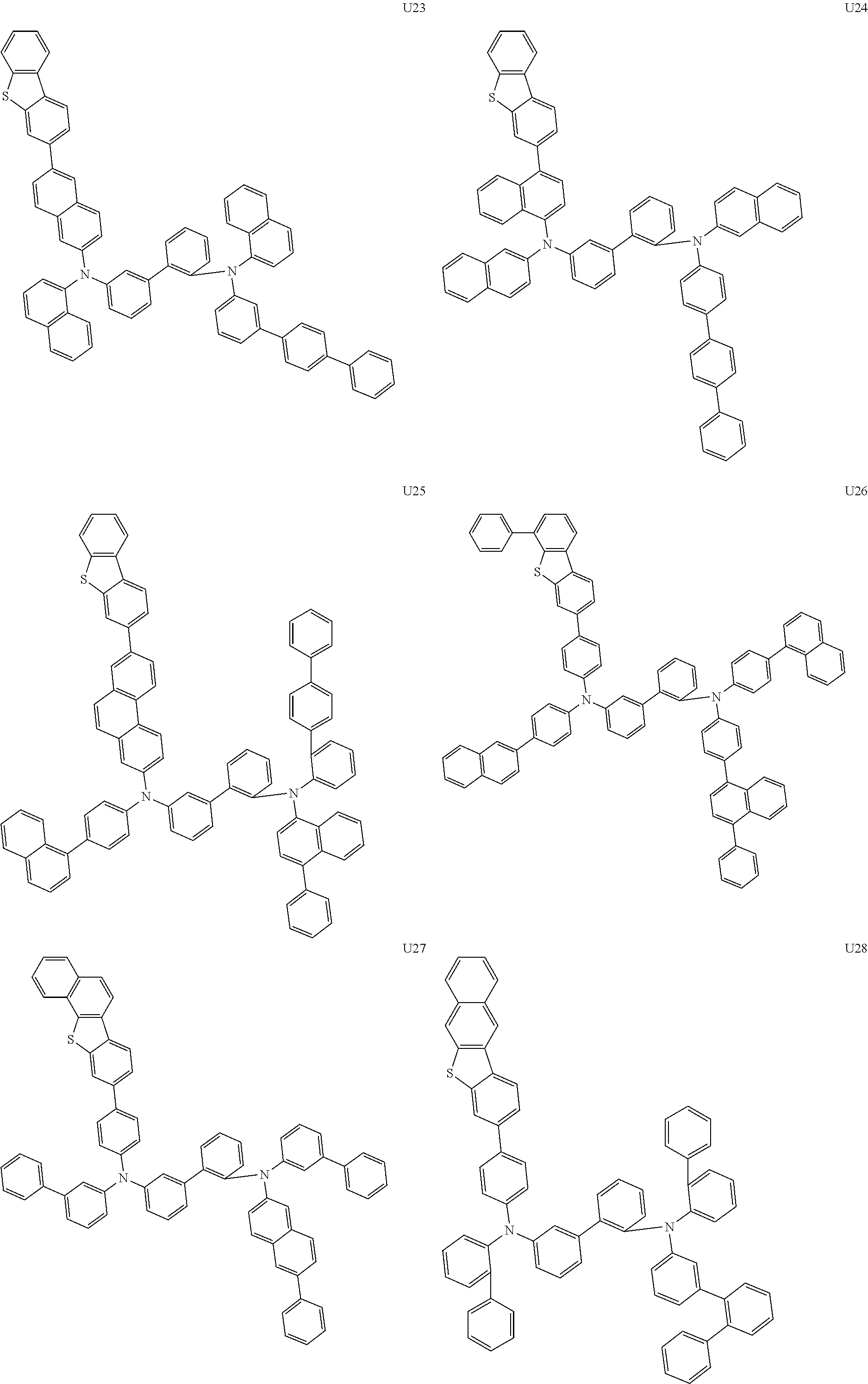

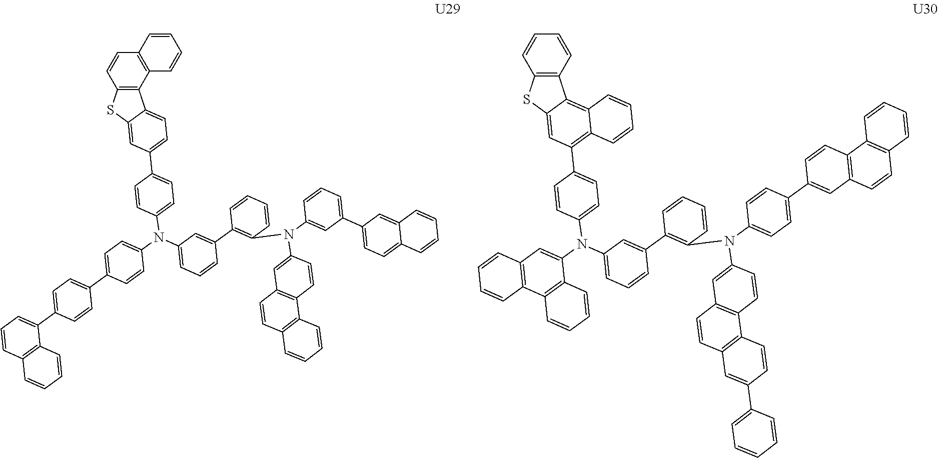 [SMILES: c1cc(-c2ccccc2N(c2cccc(-c3ccc4ccccc4c3)c2)c2ccc3c(ccc4ccccc43)c2)cc(N(c2ccc(-c3ccc(-c4cccc5ccccc45)cc3)cc2)c2ccc(-c3ccc4c(c3)sc3ccc5ccccc5c34)cc2)c1 ; c1ccc(-c2ccc3c(ccc4cc(N(c5ccc(-c6ccc7c(ccc8ccccc87)c6)cc5)c5ccccc5-c5cccc(N(c6ccc(-c7cc8sc9ccccc9c8c8ccccc78)cc6)c6cc7ccccc7c7ccccc67)c5)ccc43)c2)cc1]

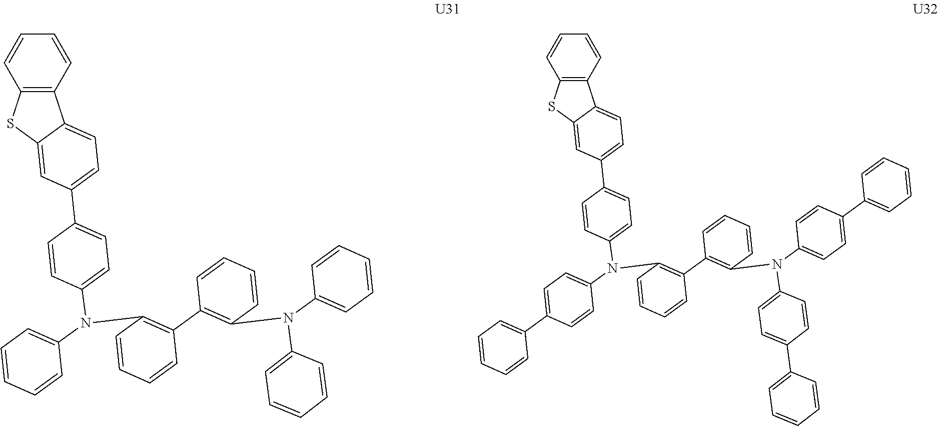 [SMILES: c1ccc(N(c2ccccc2)c2ccccc2-c2ccccc2N(c2ccccc2)c2ccc(-c3ccc4c(c3)sc3ccccc34)cc2)cc1 ; c1ccc(-c2ccc(N(c3ccc(-c4ccccc4)cc3)c3ccccc3-c3ccccc3N(c3ccc(-c4ccccc4)cc3)c3ccc(-c4ccc5c(c4)sc4ccccc45)cc3)cc2)cc1]

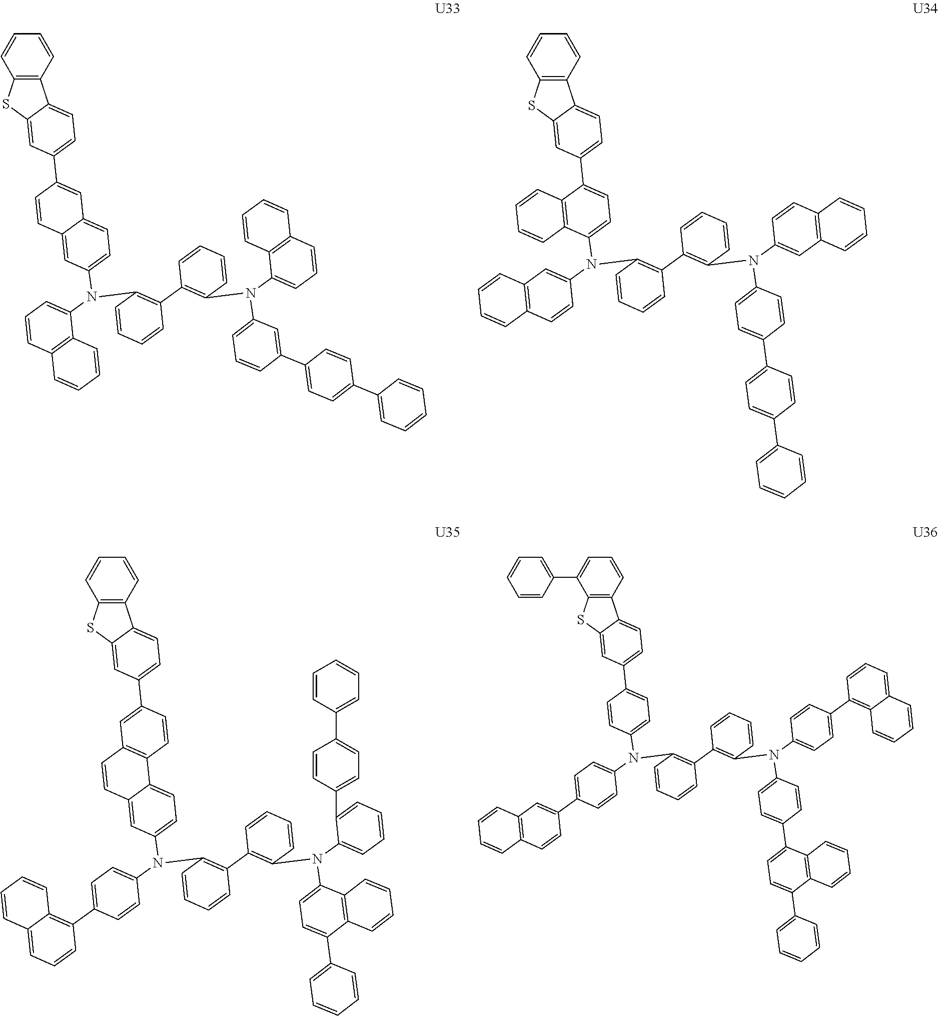

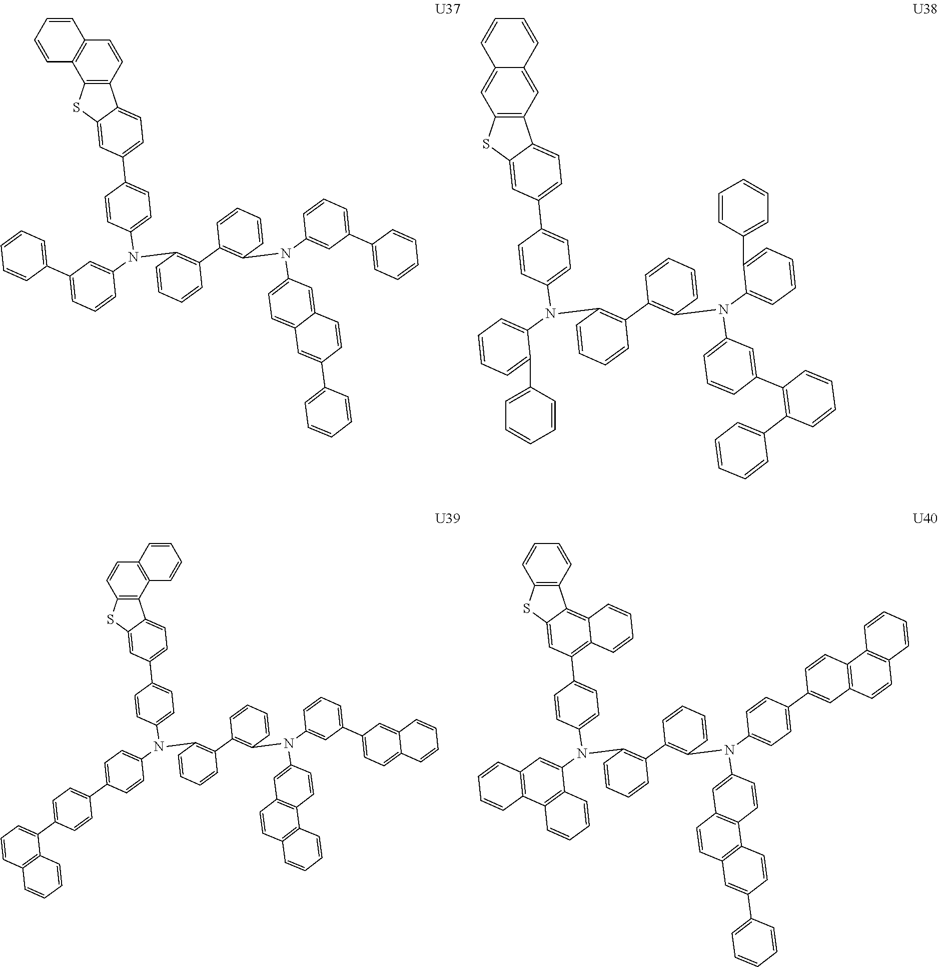

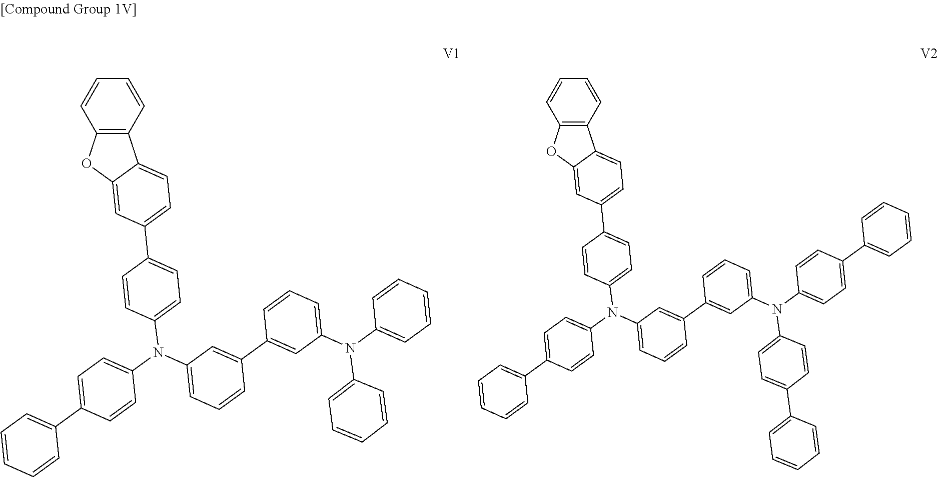 [SMILES: c1ccc(-c2ccc(N(c3ccc(-c4ccc5c(c4)oc4ccccc45)cc3)c3cccc(-c4cccc(N(c5ccccc5)c5ccccc5)c4)c3)cc2)cc1 ; c1ccc(-c2ccc(N(c3ccc(-c4ccccc4)cc3)c3cccc(-c4cccc(N(c5ccc(-c6ccccc6)cc5)c5ccc(-c6ccc7c(c6)oc6ccccc67)cc5)c4)c3)cc2)cc1]

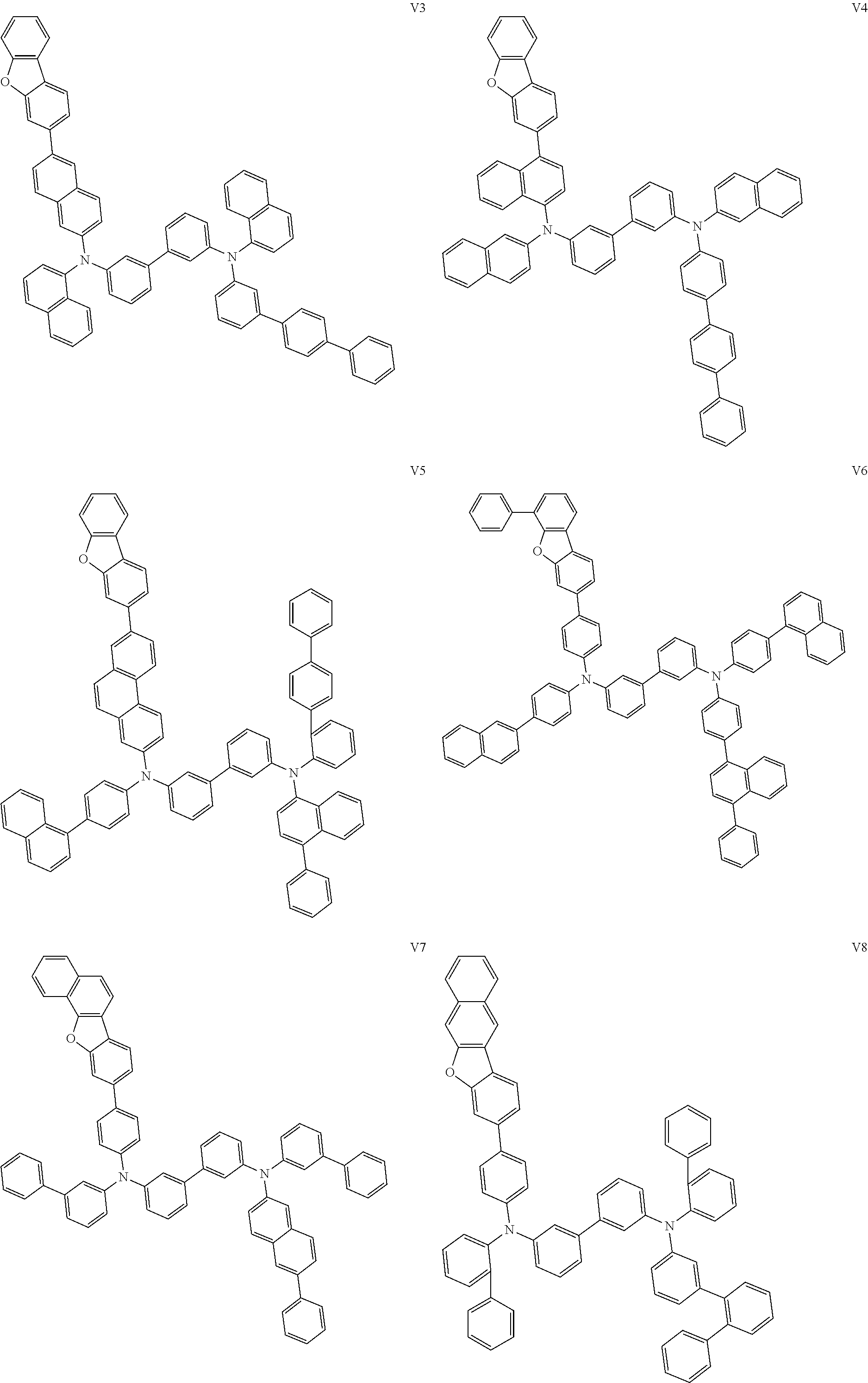

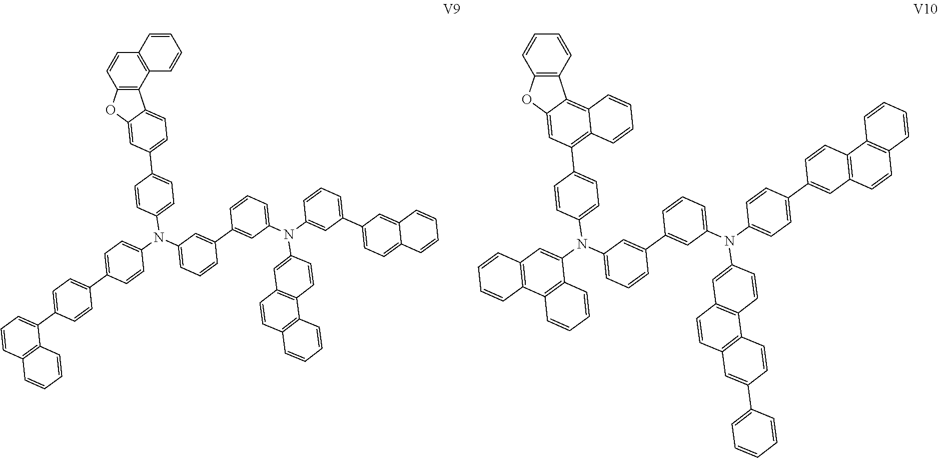 [SMILES: c1cc(-c2cccc(N(c3cccc(-c4ccc5ccccc5c4)c3)c3ccc4c(ccc5ccccc54)c3)c2)cc(N(c2ccc(-c3ccc(-c4cccc5ccccc45)cc3)cc2)c2ccc(-c3ccc4c(c3)oc3ccc5ccccc5c34)cc2)c1 ; c1ccc(-c2ccc3c(ccc4cc(N(c5ccc(-c6ccc7c(ccc8ccccc87)c6)cc5)c5cccc(-c6cccc(N(c7ccc(-c8cc9oc%10ccccc%10c9c9ccccc89)cc7)c7cc8ccccc8c8ccccc78)c6)c5)ccc43)c2)cc1]

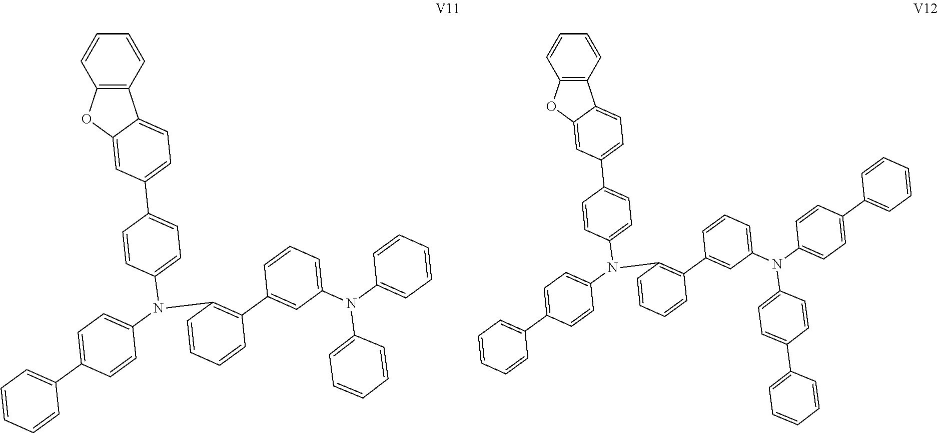 [SMILES: c1ccc(-c2ccc(N(c3ccc(-c4ccc5c(c4)oc4ccccc45)cc3)c3ccccc3-c3cccc(N(c4ccccc4)c4ccccc4)c3)cc2)cc1 ; c1ccc(-c2ccc(N(c3ccc(-c4ccccc4)cc3)c3cccc(-c4ccccc4N(c4ccc(-c5ccccc5)cc4)c4ccc(-c5ccc6c(c5)oc5ccccc56)cc4)c3)cc2)cc1]

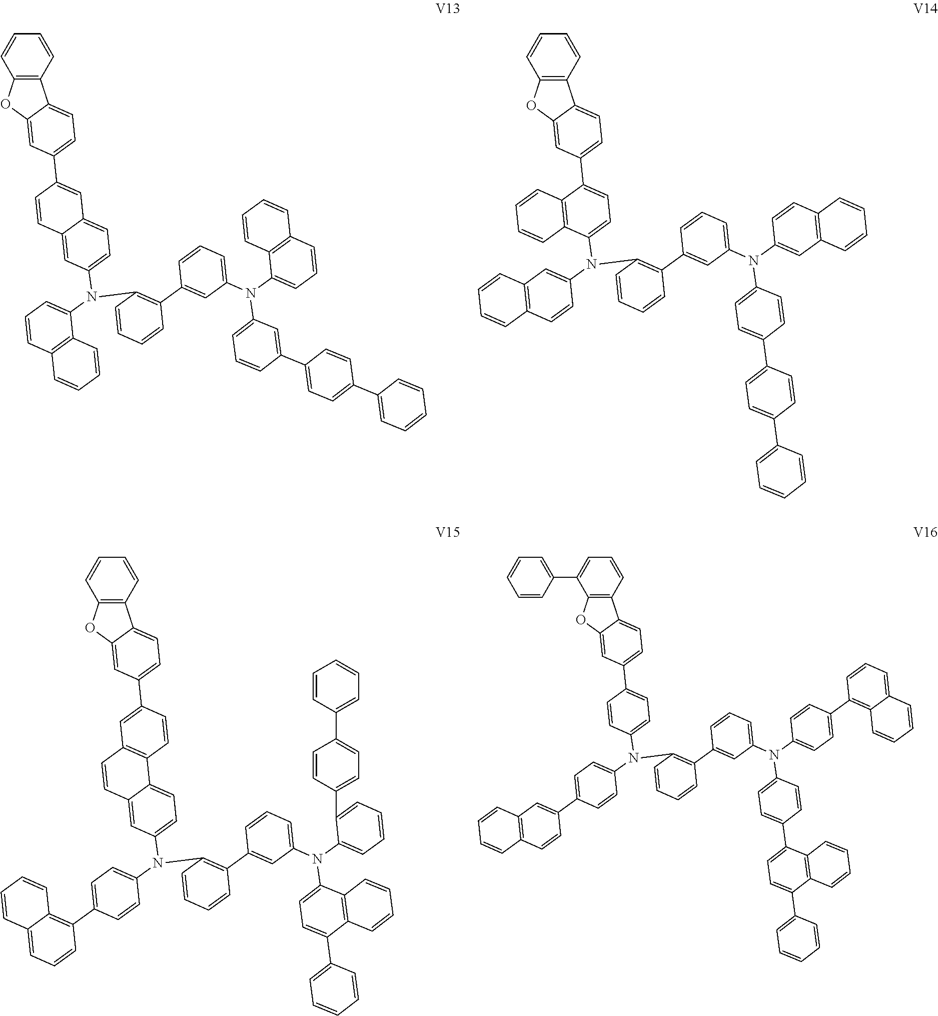

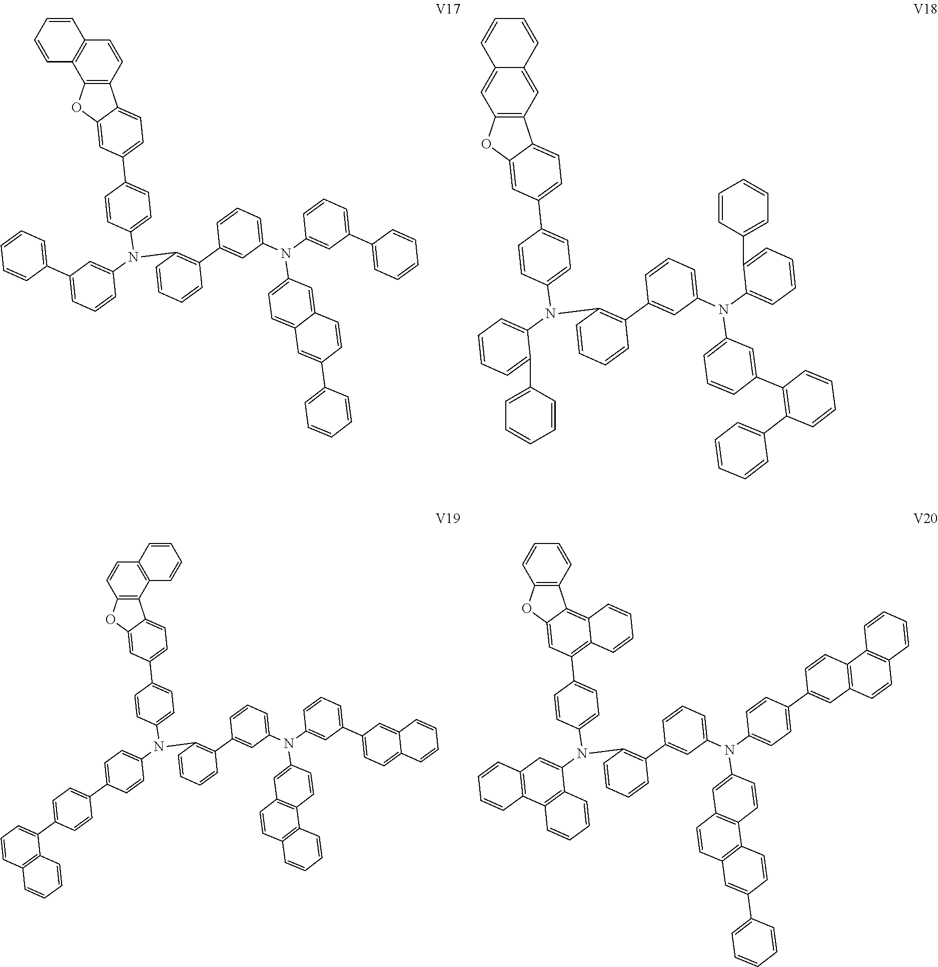

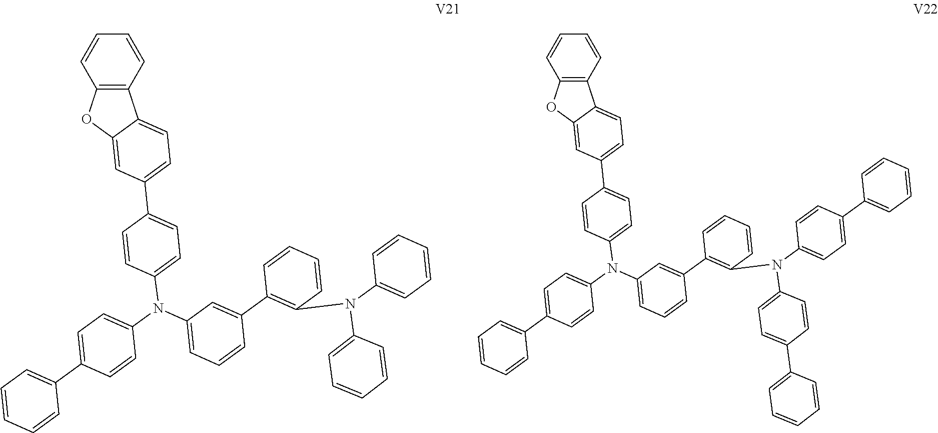 [SMILES: c1ccc(-c2ccc(N(c3ccc(-c4ccc5c(c4)oc4ccccc45)cc3)c3cccc(-c4ccccc4N(c4ccccc4)c4ccccc4)c3)cc2)cc1 ; c1ccc(-c2ccc(N(c3ccc(-c4ccc5c(c4)oc4ccccc45)cc3)c3cccc(-c4ccccc4N(c4ccc(-c5ccccc5)cc4)c4ccc(-c5ccccc5)cc4)c3)cc2)cc1]

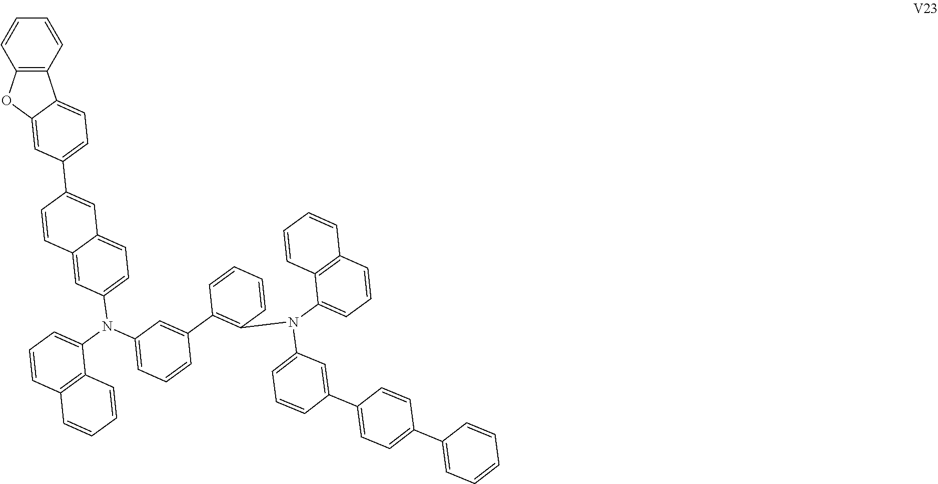 [SMILES: c1ccc(-c2ccc(-c3cccc(N(c4ccccc4-c4cccc(N(c5ccc6cc(-c7ccc8c(c7)oc7ccccc78)ccc6c5)c5cccc6ccccc56)c4)c4cccc5ccccc45)c3)cc2)cc1]

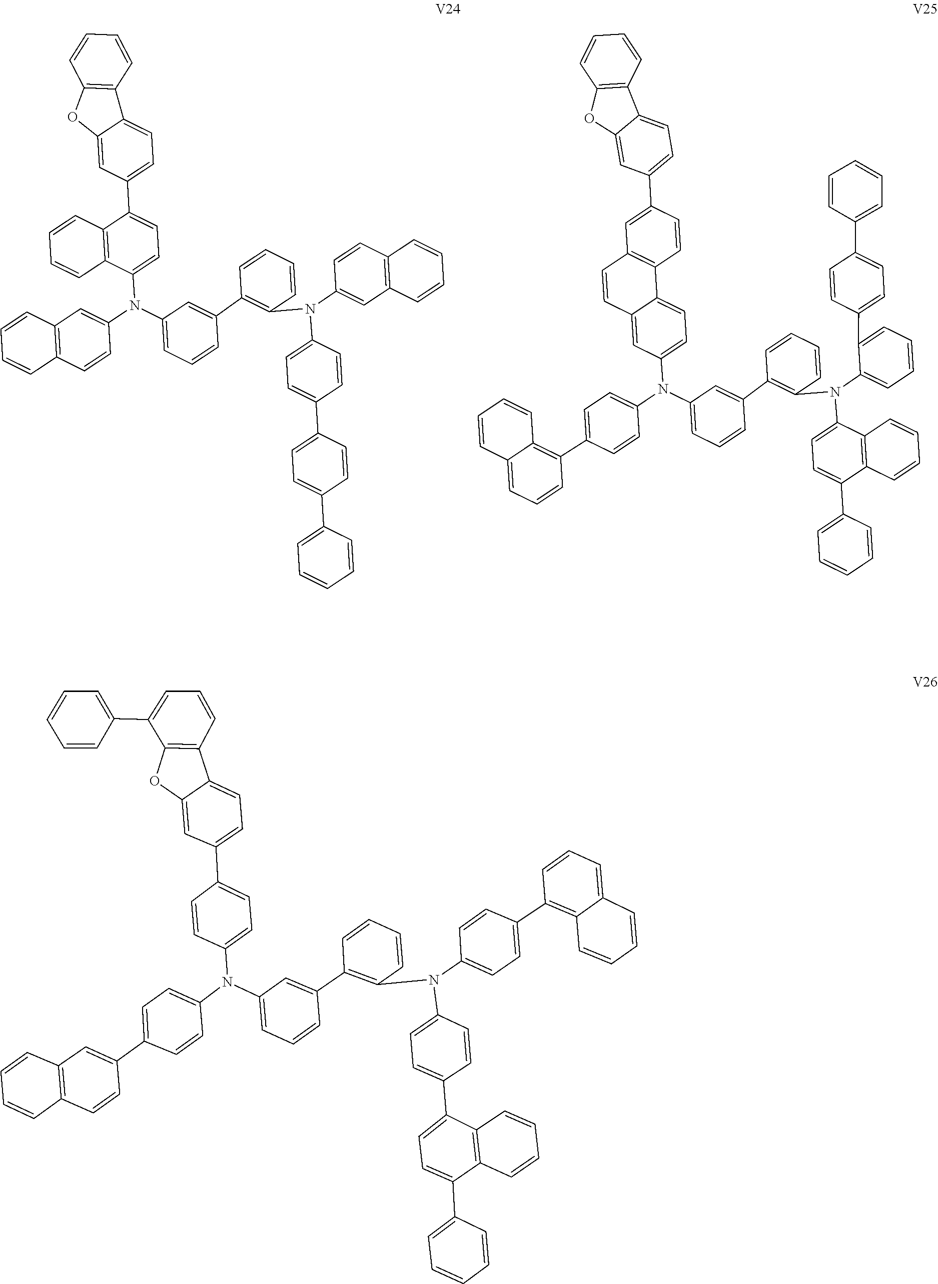 [SMILES: c1ccc(-c2ccc(-c3ccc(N(c4ccc5ccccc5c4)c4ccccc4-c4cccc(N(c5ccc6ccccc6c5)c5ccc(-c6ccc7c(c6)oc6ccccc67)c6ccccc56)c4)cc3)cc2)cc1 ; c1ccc(-c2ccc(-c3ccccc3N(c3ccccc3-c3cccc(N(c4ccc(-c5cccc6ccccc56)cc4)c4ccc5c(ccc6cc(-c7ccc8c(c7)oc7ccccc78)ccc65)c4)c3)c3ccc(-c4ccccc4)c4ccccc34)cc2)cc1 ; c1ccc(-c2ccc(-c3ccc(N(c4ccc(-c5cccc6ccccc56)cc4)c4ccccc4-c4cccc(N(c5ccc(-c6ccc7ccccc7c6)cc5)c5ccc(-c6ccc7c(c6)oc6c(-c8ccccc8)cccc67)cc5)c4)cc3)c3ccccc23)cc1]

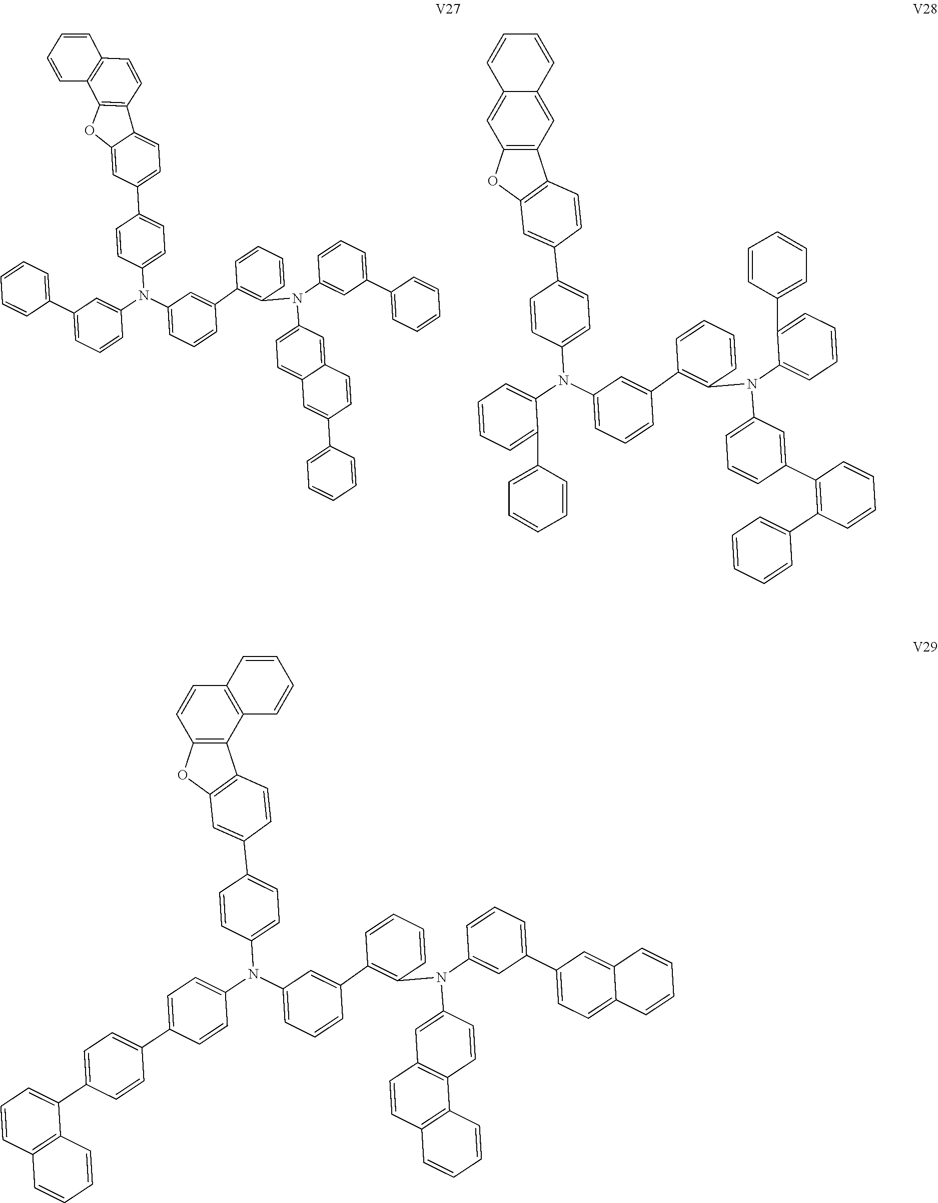 [SMILES: c1ccc(-c2cccc(N(c3ccc(-c4ccc5c(c4)oc4c6ccccc6ccc54)cc3)c3cccc(-c4ccccc4N(c4cccc(-c5ccccc5)c4)c4ccc5cc(-c6ccccc6)ccc5c4)c3)c2)cc1 ; c1ccc(-c2ccccc2-c2cccc(N(c3ccccc3-c3ccccc3)c3ccccc3-c3cccc(N(c4ccc(-c5ccc6c(c5)oc5cc7ccccc7cc56)cc4)c4ccccc4-c4ccccc4)c3)c2)cc1 ; c1cc(-c2ccccc2N(c2cccc(-c3ccc4ccccc4c3)c2)c2ccc3c(ccc4ccccc43)c2)cc(N(c2ccc(-c3ccc(-c4cccc5ccccc45)cc3)cc2)c2ccc(-c3ccc4c(c3)oc3ccc5ccccc5c34)cc2)c1]

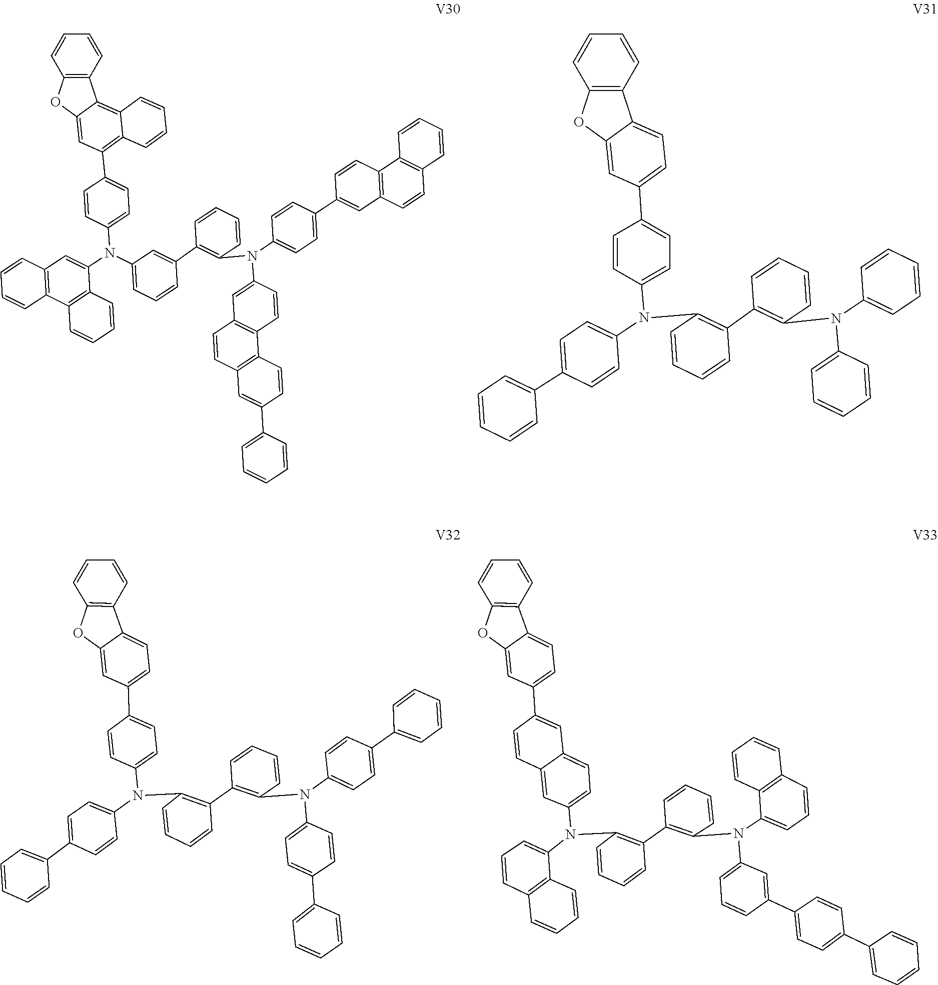 [SMILES: c1ccc(-c2ccc3c(ccc4cc(N(c5ccc(-c6ccc7c(ccc8ccccc87)c6)cc5)c5ccccc5-c5cccc(N(c6ccc(-c7cc8oc9ccccc9c8c8ccccc78)cc6)c6cc7ccccc7c7ccccc67)c5)ccc43)c2)cc1 ; c1ccc(-c2ccc(N(c3ccc(-c4ccc5c(c4)oc4ccccc45)cc3)c3ccccc3-c3ccccc3N(c3ccccc3)c3ccccc3)cc2)cc1 ; c1ccc(-c2ccc(N(c3ccc(-c4ccccc4)cc3)c3ccccc3-c3ccccc3N(c3ccc(-c4ccccc4)cc3)c3ccc(-c4ccc5c(c4)oc4ccccc45)cc3)cc2)cc1 ; c1ccc(-c2ccc(-c3cccc(N(c4ccccc4-c4ccccc4N(c4ccc5cc(-c6ccc7c(c6)oc6ccccc67)ccc5c4)c4cccc5ccccc45)c4cccc5ccccc45)c3)cc2)cc1]

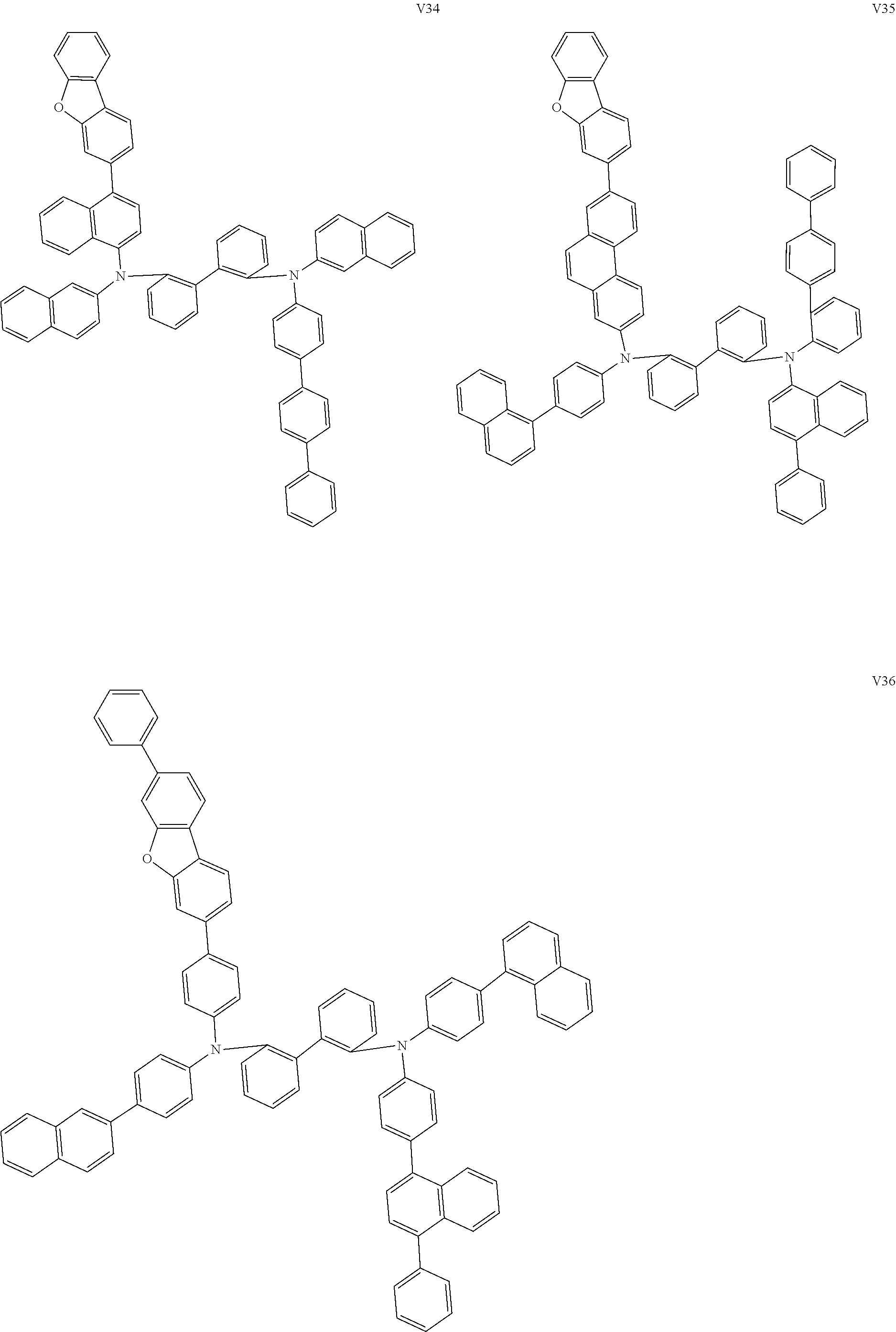 [SMILES: c1ccc(-c2ccc(-c3ccc(N(c4ccc5ccccc5c4)c4ccccc4-c4ccccc4N(c4ccc5ccccc5c4)c4ccc(-c5ccc6c(c5)oc5ccccc56)c5ccccc45)cc3)cc2)cc1 ; c1ccc(-c2ccc(-c3ccccc3N(c3ccccc3-c3ccccc3N(c3ccc(-c4cccc5ccccc45)cc3)c3ccc4c(ccc5cc(-c6ccc7c(c6)oc6ccccc67)ccc54)c3)c3ccc(-c4ccccc4)c4ccccc34)cc2)cc1 ; c1ccc(-c2ccc3c(c2)oc2cc(-c4ccc(N(c5ccc(-c6ccc7ccccc7c6)cc5)c5ccccc5-c5ccccc5N(c5ccc(-c6cccc7ccccc67)cc5)c5ccc(-c6ccc(-c7ccccc7)c7ccccc67)cc5)cc4)ccc23)cc1]

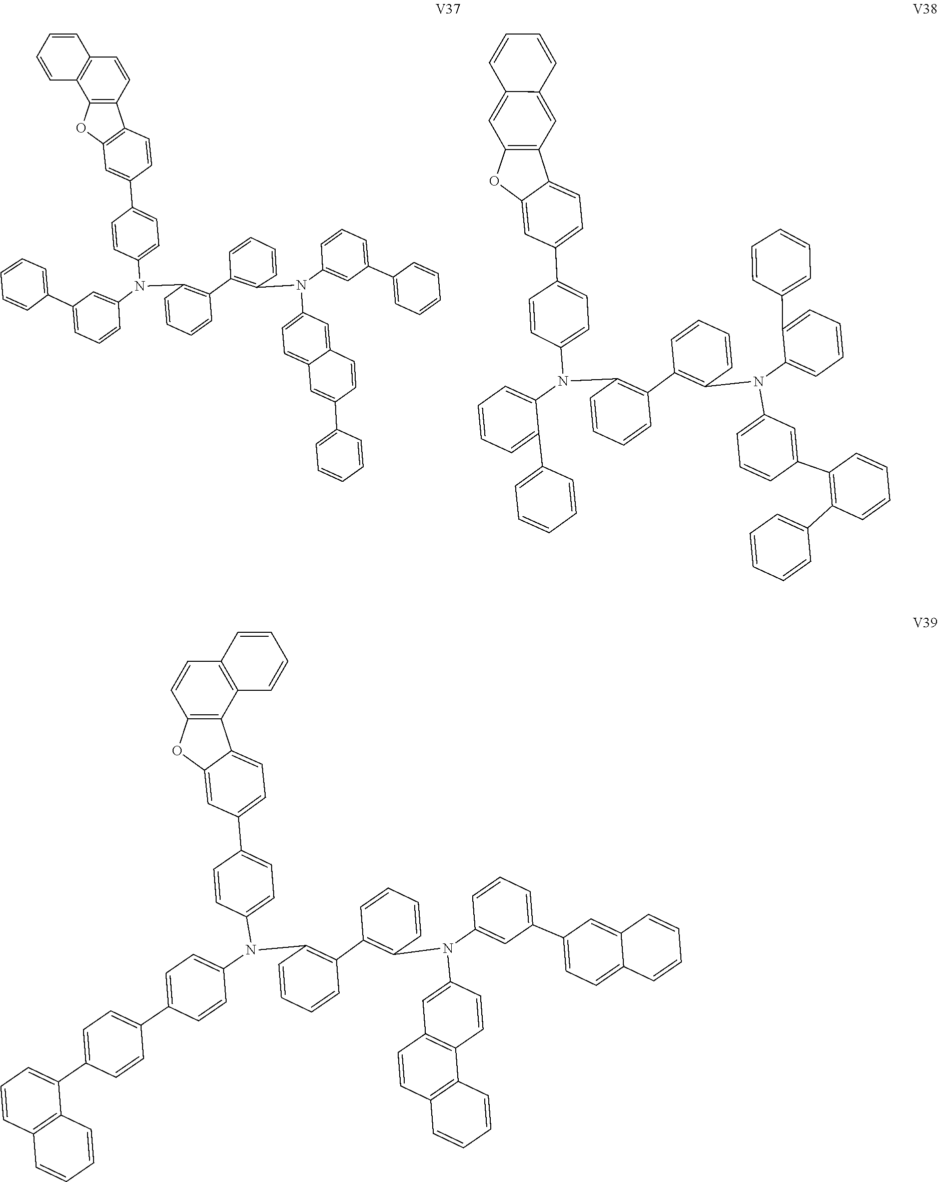 [SMILES: c1ccc(-c2cccc(N(c3ccc(-c4ccc5c(c4)oc4c6ccccc6ccc54)cc3)c3ccccc3-c3ccccc3N(c3cccc(-c4ccccc4)c3)c3ccc4cc(-c5ccccc5)ccc4c3)c2)cc1 ; c1ccc(-c2ccccc2-c2cccc(N(c3ccccc3-c3ccccc3)c3ccccc3-c3ccccc3N(c3ccc(-c4ccc5c(c4)oc4cc6ccccc6cc45)cc3)c3ccccc3-c3ccccc3)c2)cc1 ; c1cc(-c2ccc3ccccc3c2)cc(N(c2ccc3c(ccc4ccccc43)c2)c2ccccc2-c2ccccc2N(c2ccc(-c3ccc(-c4cccc5ccccc45)cc3)cc2)c2ccc(-c3ccc4c(c3)oc3ccc5ccccc5c34)cc2)c1]

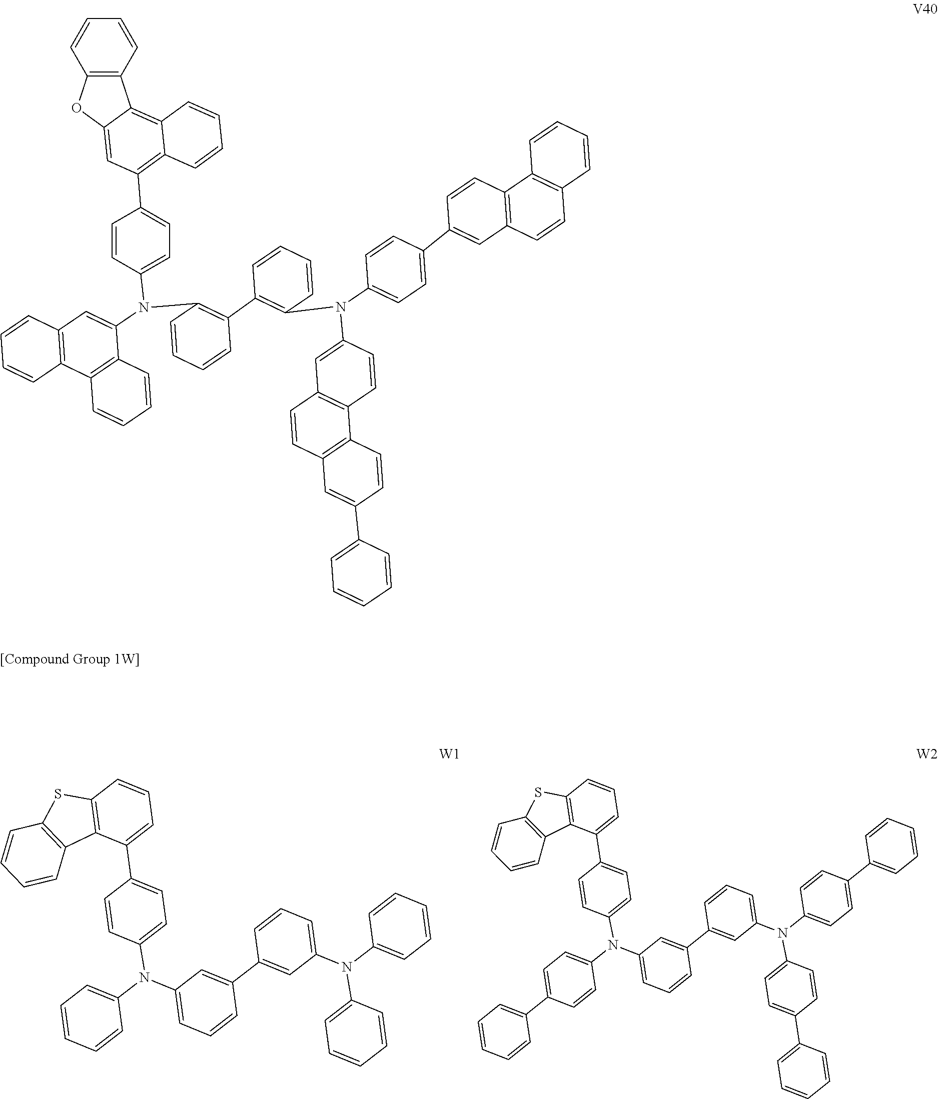 [SMILES: c1ccc(-c2ccc3c(ccc4cc(N(c5ccc(-c6ccc7c(ccc8ccccc87)c6)cc5)c5ccccc5-c5ccccc5N(c5ccc(-c6cc7oc8ccccc8c7c7ccccc67)cc5)c5cc6ccccc6c6ccccc56)ccc43)c2)cc1 ; c1ccc(N(c2ccccc2)c2cccc(-c3cccc(N(c4ccccc4)c4ccc(-c5cccc6sc7ccccc7c56)cc4)c3)c2)cc1 ; c1ccc(-c2ccc(N(c3ccc(-c4ccccc4)cc3)c3cccc(-c4cccc(N(c5ccc(-c6ccccc6)cc5)c5ccc(-c6cccc7sc8ccccc8c67)cc5)c4)c3)cc2)cc1]

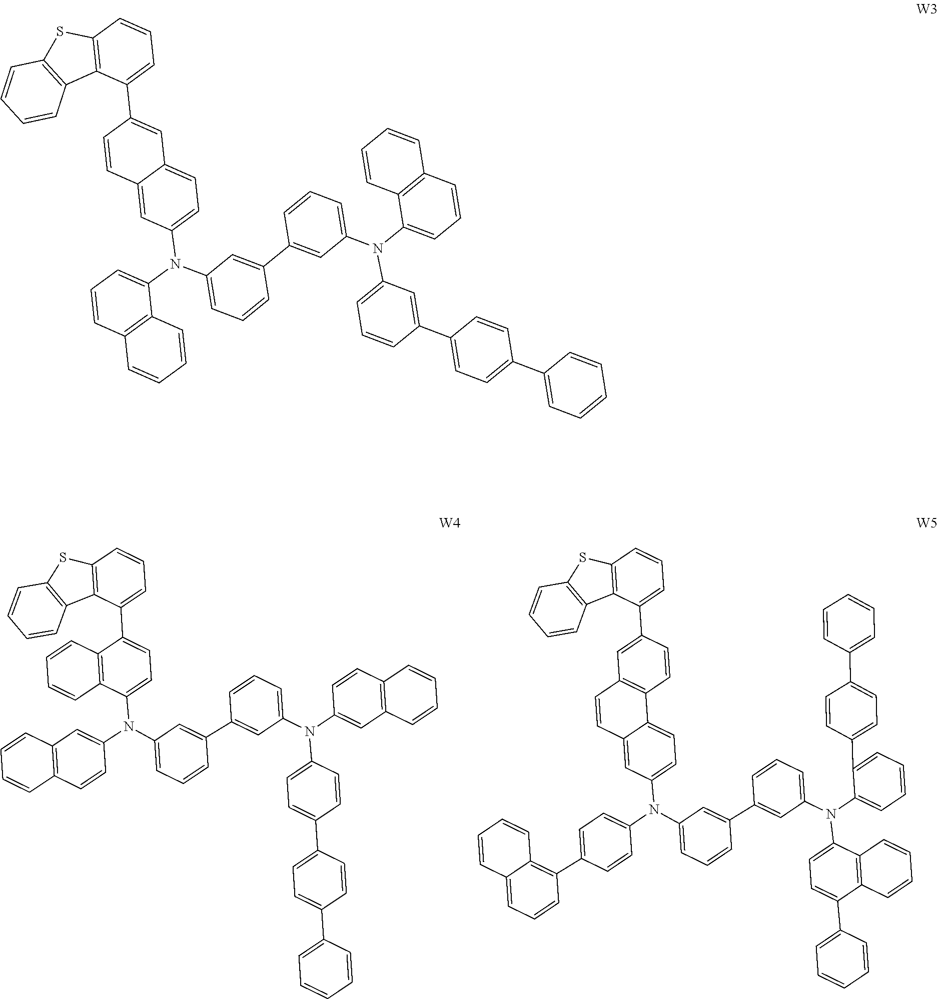 [SMILES: c1ccc(-c2ccc(-c3cccc(N(c4cccc(-c5cccc(N(c6ccc7cc(-c8cccc9sc%10ccccc%10c89)ccc7c6)c6cccc7ccccc67)c5)c4)c4cccc5ccccc45)c3)cc2)cc1 ; c1ccc(-c2ccc(-c3ccc(N(c4cccc(-c5cccc(N(c6ccc7ccccc7c6)c6ccc(-c7cccc8sc9ccccc9c78)c7ccccc67)c5)c4)c4ccc5ccccc5c4)cc3)cc2)cc1 ; c1ccc(-c2ccc(-c3ccccc3N(c3cccc(-c4cccc(N(c5ccc(-c6cccc7ccccc67)cc5)c5ccc6c(ccc7cc(-c8cccc9sc%10ccccc%10c89)ccc76)c5)c4)c3)c3ccc(-c4ccccc4)c4ccccc34)cc2)cc1]

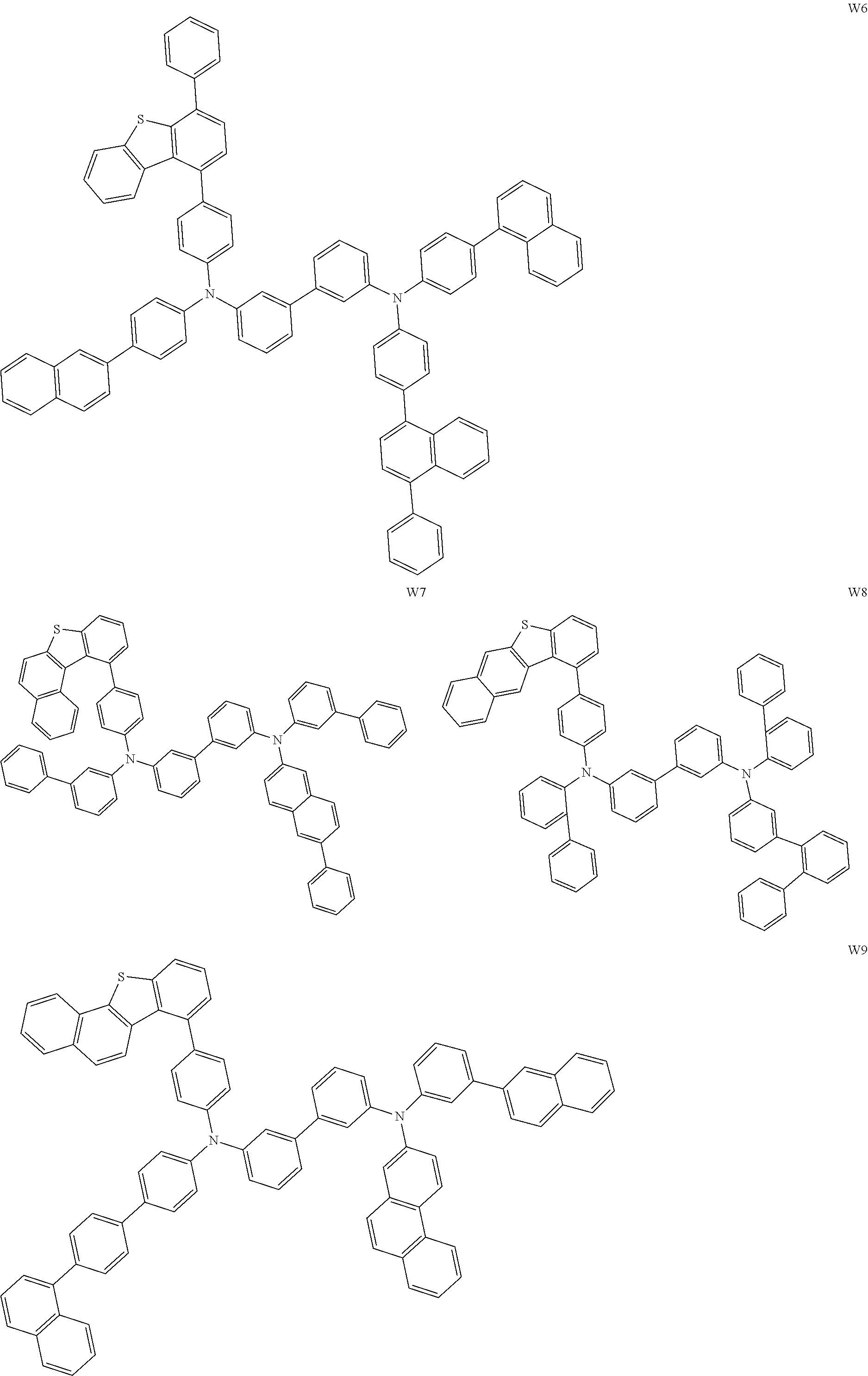

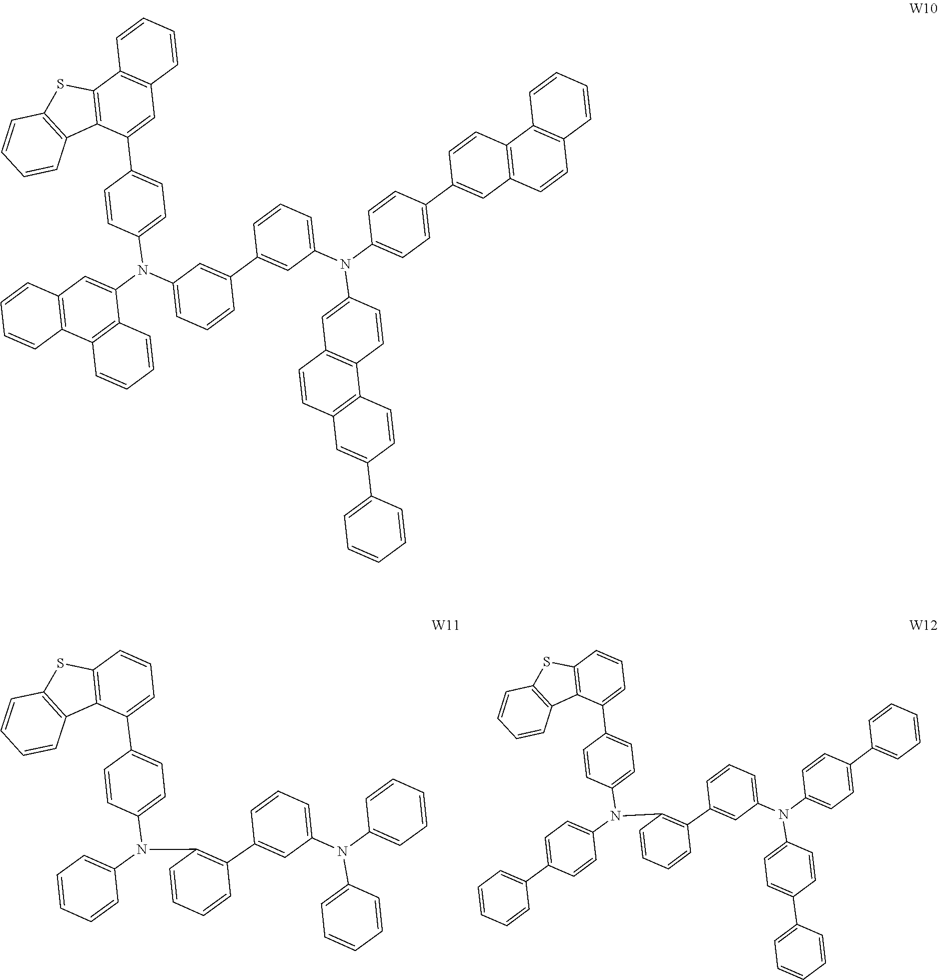 [SMILES: c1ccc(-c2ccc3c(ccc4cc(N(c5ccc(-c6ccc7c(ccc8ccccc87)c6)cc5)c5cccc(-c6cccc(N(c7ccc(-c8cc9ccccc9c9sc%10ccccc%10c89)cc7)c7cc8ccccc8c8ccccc78)c6)c5)ccc43)c2)cc1 ; c1ccc(N(c2ccccc2)c2cccc(-c3ccccc3N(c3ccccc3)c3ccc(-c4cccc5sc6ccccc6c45)cc3)c2)cc1 ; c1ccc(-c2ccc(N(c3ccc(-c4ccccc4)cc3)c3cccc(-c4ccccc4N(c4ccc(-c5ccccc5)cc4)c4ccc(-c5cccc6sc7ccccc7c56)cc4)c3)cc2)cc1]

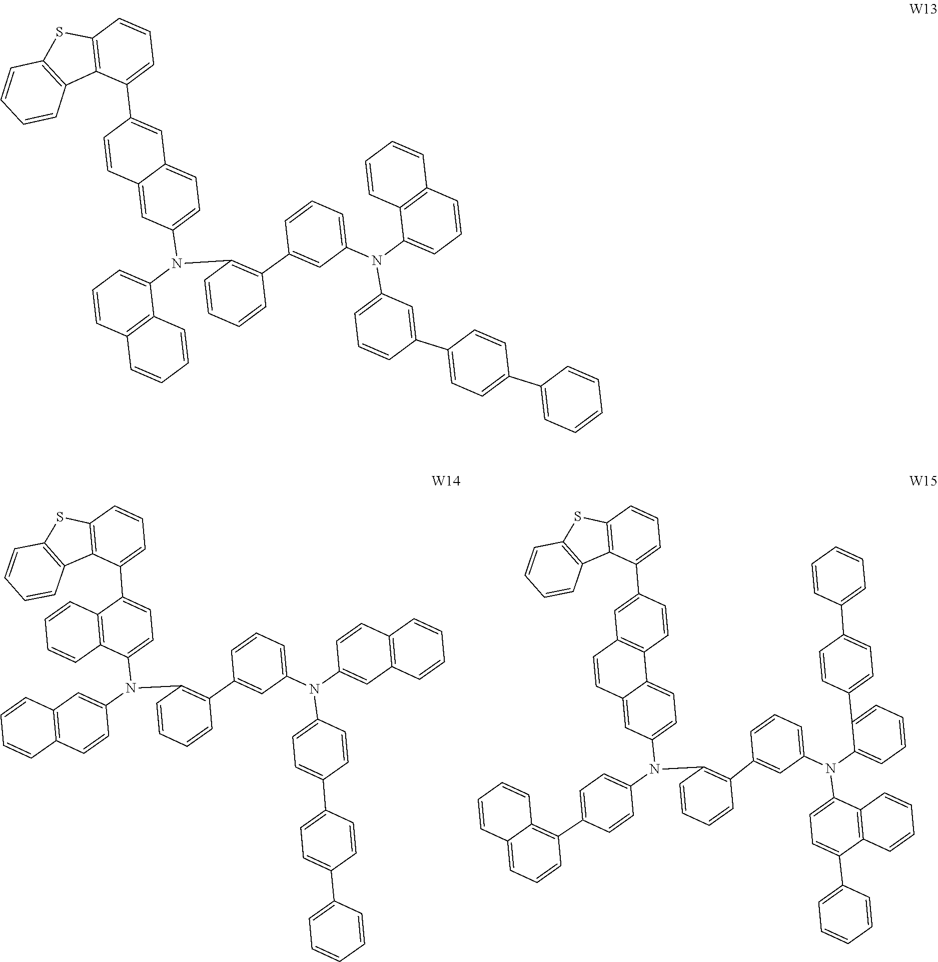 [SMILES: c1ccc(-c2ccc(-c3cccc(N(c4cccc(-c5ccccc5N(c5ccc6cc(-c7cccc8sc9ccccc9c78)ccc6c5)c5cccc6ccccc56)c4)c4cccc5ccccc45)c3)cc2)cc1 ; c1ccc(-c2ccc(-c3ccc(N(c4cccc(-c5ccccc5N(c5ccc6ccccc6c5)c5ccc(-c6cccc7sc8ccccc8c67)c6ccccc56)c4)c4ccc5ccccc5c4)cc3)cc2)cc1 ; c1ccc(-c2ccc(-c3ccccc3N(c3cccc(-c4ccccc4N(c4ccc(-c5cccc6ccccc56)cc4)c4ccc5c(ccc6cc(-c7cccc8sc9ccccc9c78)ccc65)c4)c3)c3ccc(-c4ccccc4)c4ccccc34)cc2)cc1]

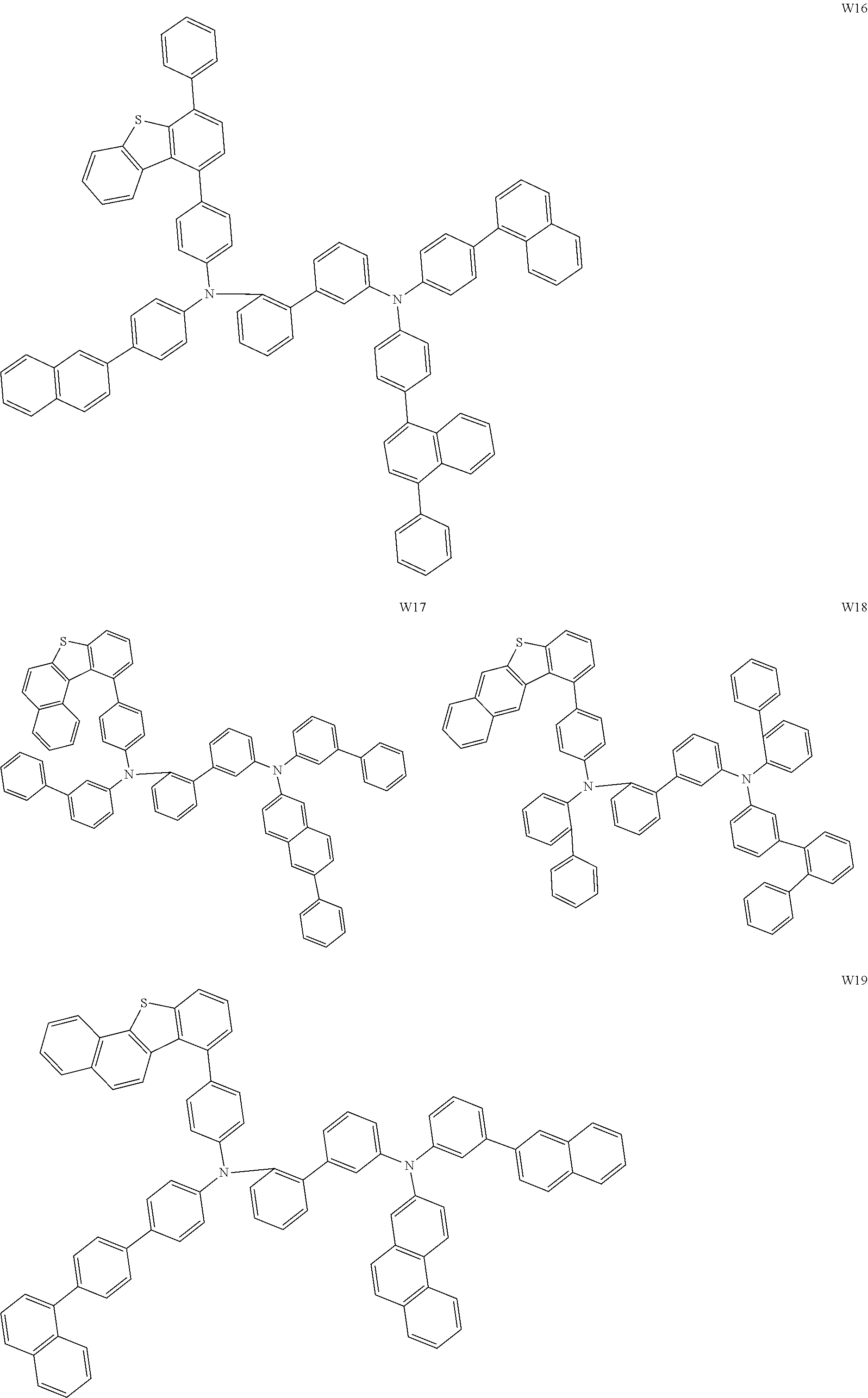

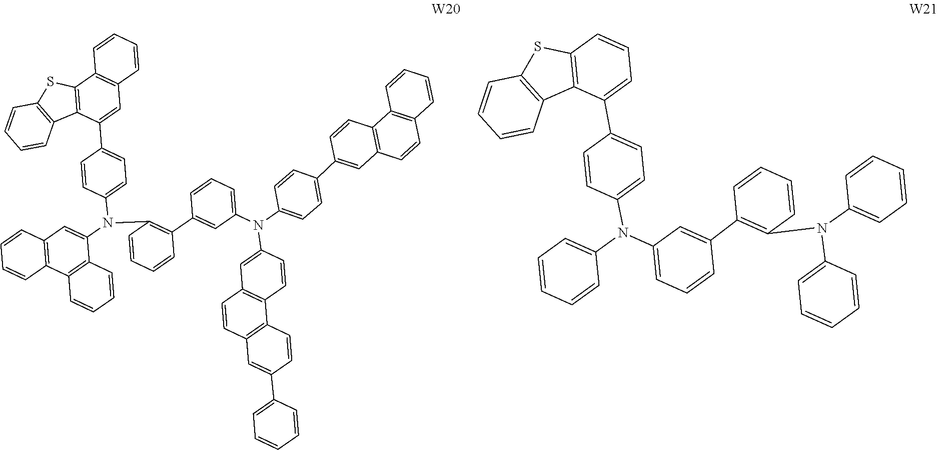 [SMILES: c1ccc(-c2ccc3c(ccc4cc(N(c5ccc(-c6ccc7c(ccc8ccccc87)c6)cc5)c5cccc(-c6ccccc6N(c6ccc(-c7cc8ccccc8c8sc9ccccc9c78)cc6)c6cc7ccccc7c7ccccc67)c5)ccc43)c2)cc1 ; c1ccc(N(c2ccc(-c3cccc4sc5ccccc5c34)cc2)c2cccc(-c3ccccc3N(c3ccccc3)c3ccccc3)c2)cc1]

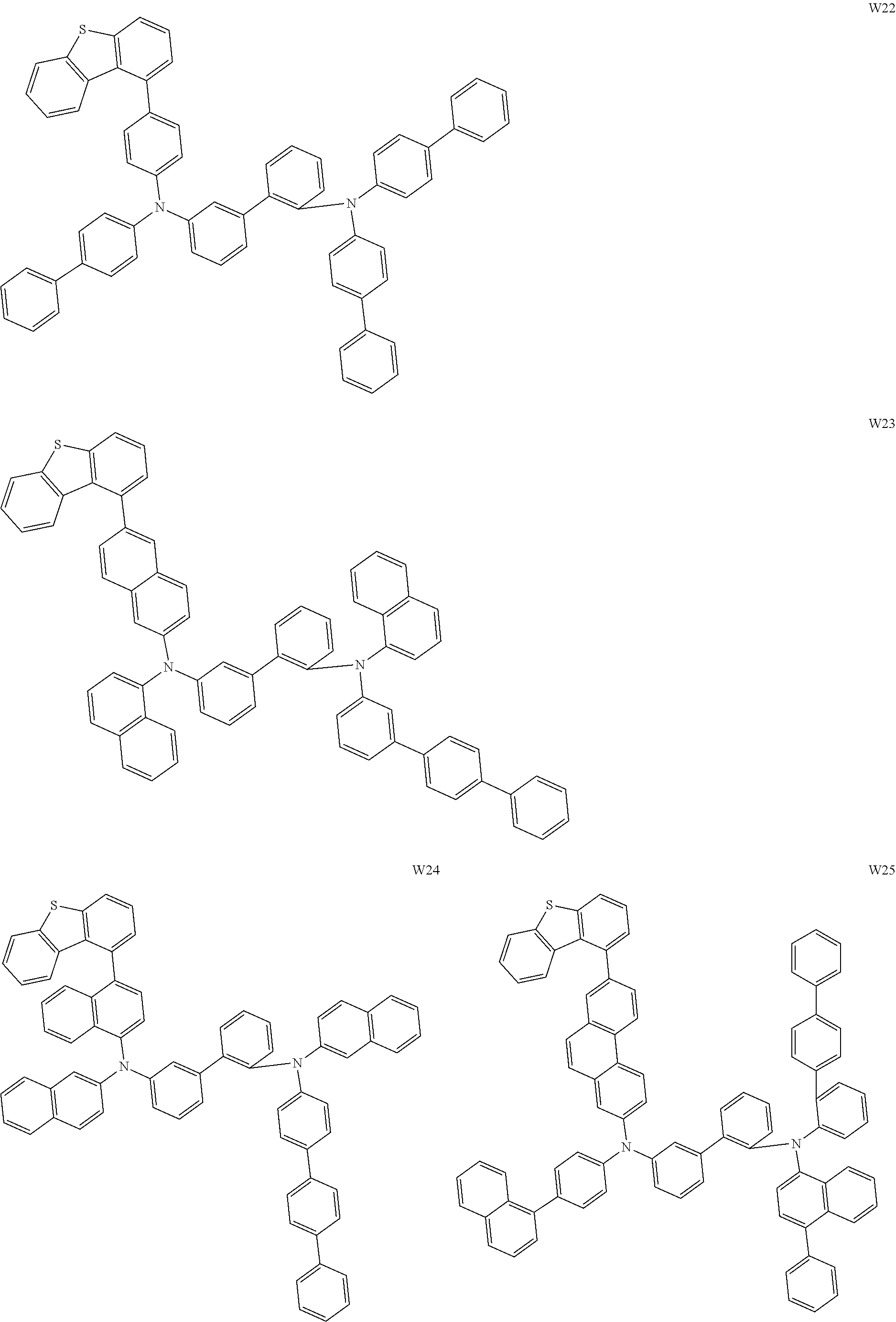

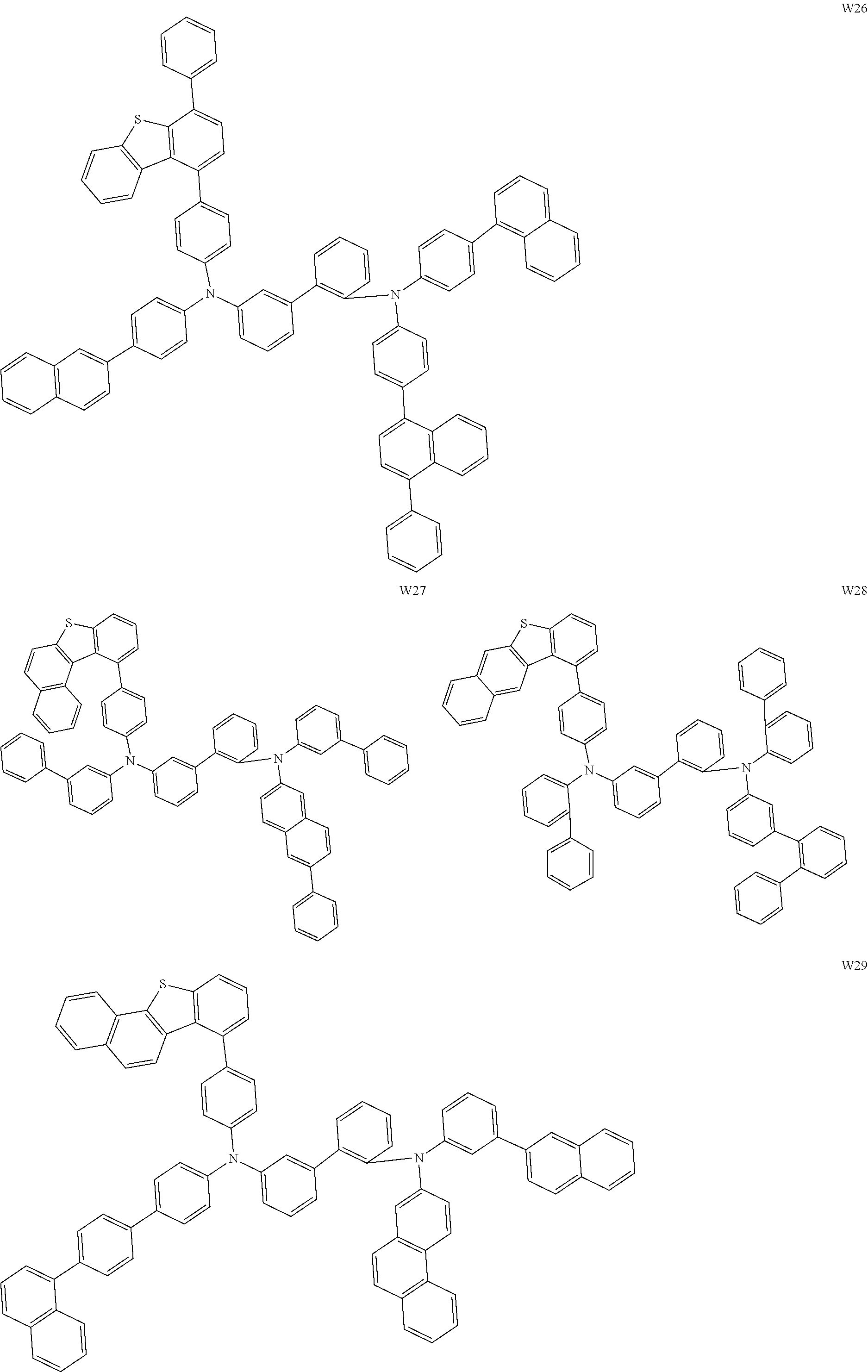

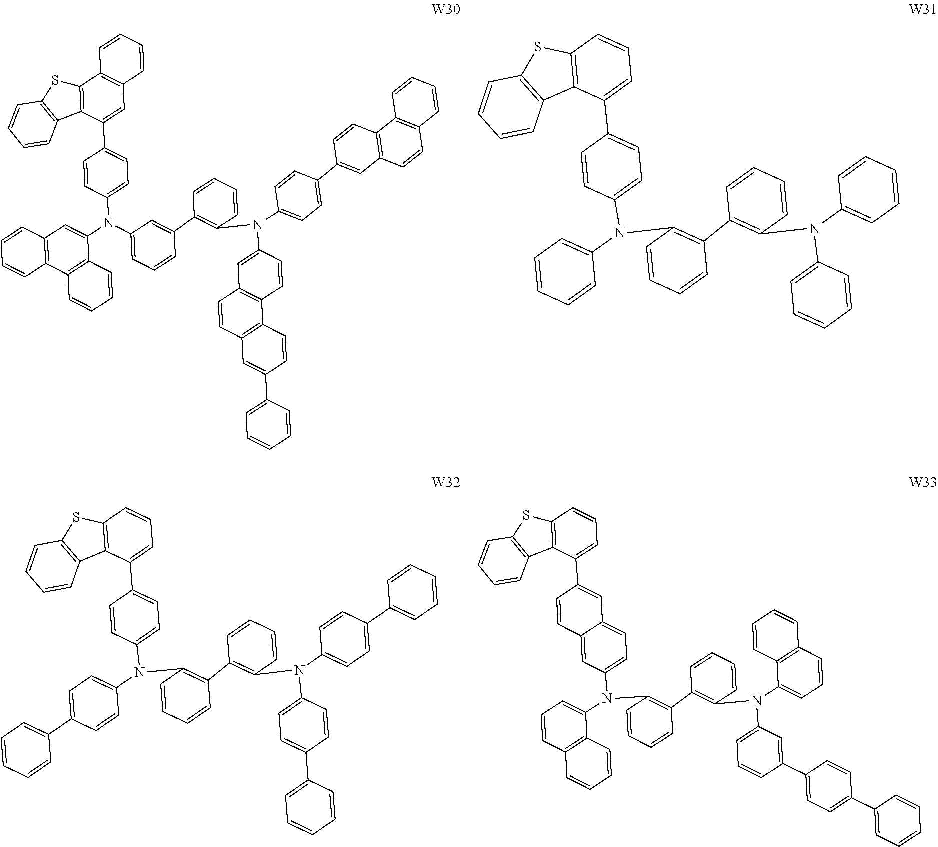 [SMILES: c1ccc(-c2ccc3c(ccc4cc(N(c5ccc(-c6ccc7c(ccc8ccccc87)c6)cc5)c5ccccc5-c5cccc(N(c6ccc(-c7cc8ccccc8c8sc9ccccc9c78)cc6)c6cc7ccccc7c7ccccc67)c5)ccc43)c2)cc1 ; c1ccc(N(c2ccccc2)c2ccccc2-c2ccccc2N(c2ccccc2)c2ccc(-c3cccc4sc5ccccc5c34)cc2)cc1 ; c1ccc(-c2ccc(N(c3ccc(-c4ccccc4)cc3)c3ccccc3-c3ccccc3N(c3ccc(-c4ccccc4)cc3)c3ccc(-c4cccc5sc6ccccc6c45)cc3)cc2)cc1 ; c1ccc(-c2ccc(-c3cccc(N(c4ccccc4-c4ccccc4N(c4ccc5cc(-c6cccc7sc8ccccc8c67)ccc5c4)c4cccc5ccccc45)c4cccc5ccccc45)c3)cc2)cc1]

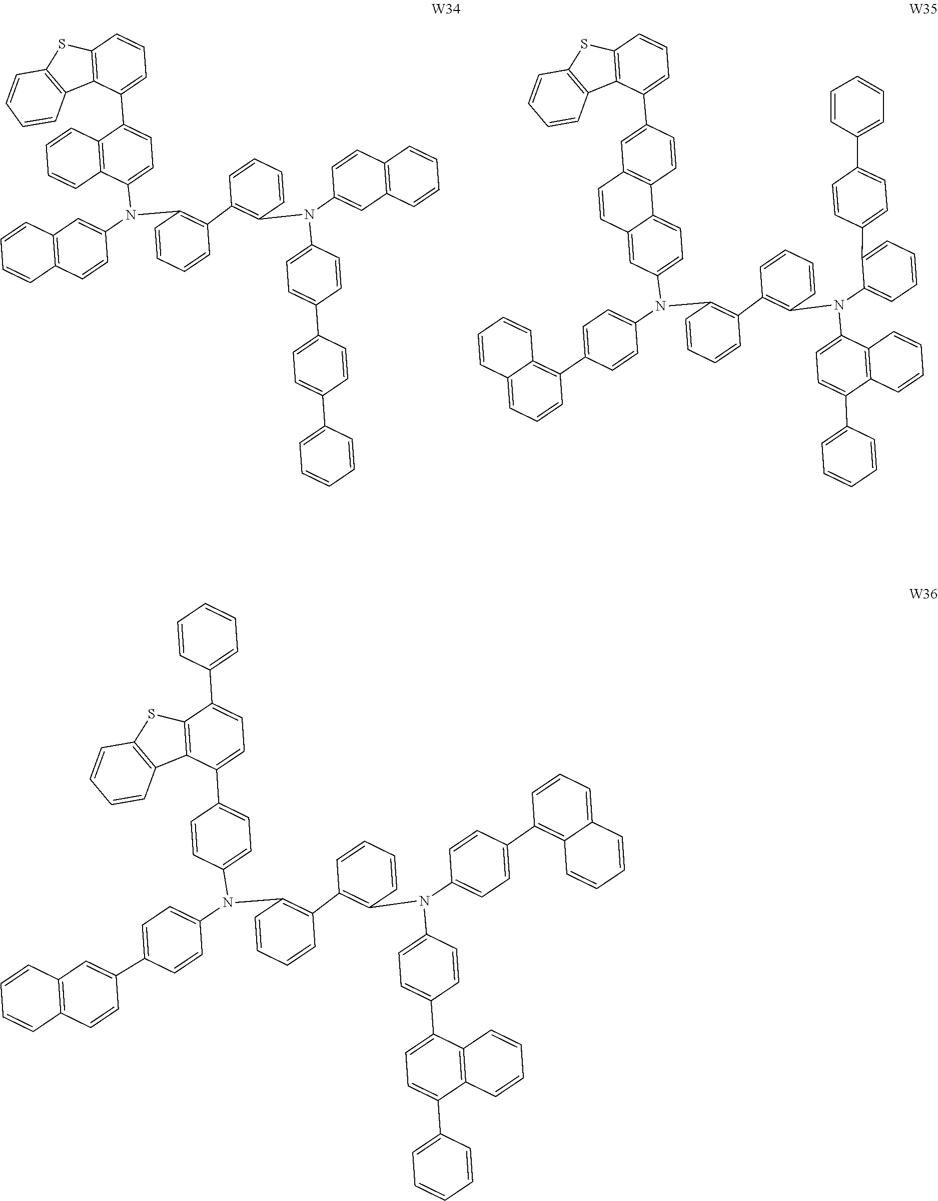 [SMILES: c1ccc(-c2ccc(-c3ccc(N(c4ccc5ccccc5c4)c4ccccc4-c4ccccc4N(c4ccc5ccccc5c4)c4ccc(-c5cccc6sc7ccccc7c56)c5ccccc45)cc3)cc2)cc1 ; c1ccc(-c2ccc(-c3ccccc3N(c3ccccc3-c3ccccc3N(c3ccc(-c4cccc5ccccc45)cc3)c3ccc4c(ccc5cc(-c6cccc7sc8ccccc8c67)ccc54)c3)c3ccc(-c4ccccc4)c4ccccc34)cc2)cc1 ; c1ccc(-c2ccc(-c3ccc(N(c4ccc(-c5cccc6ccccc56)cc4)c4ccccc4-c4ccccc4N(c4ccc(-c5ccc6ccccc6c5)cc4)c4ccc(-c5ccc(-c6ccccc6)c6sc7ccccc7c56)cc4)cc3)c3ccccc23)cc1]

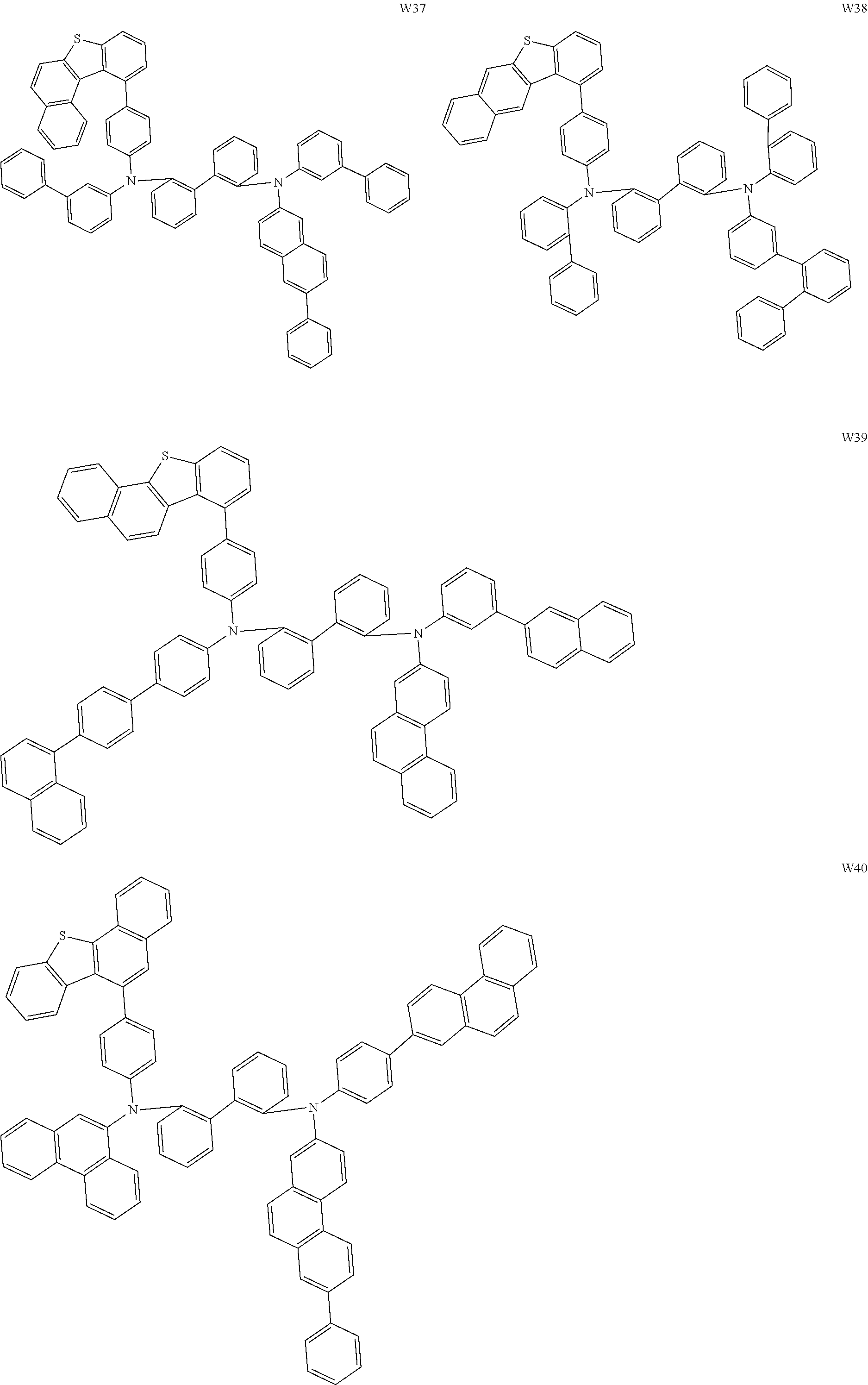

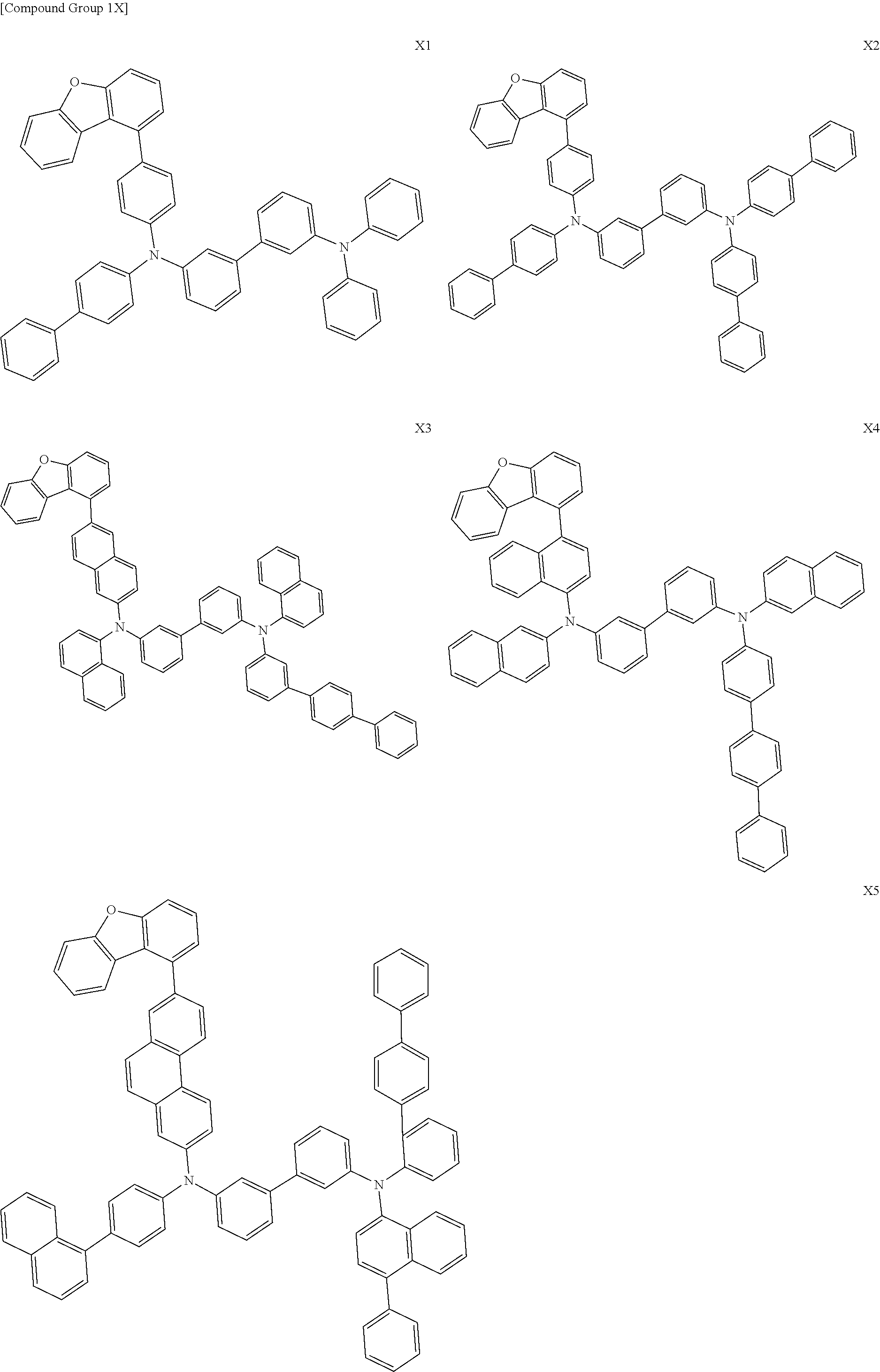

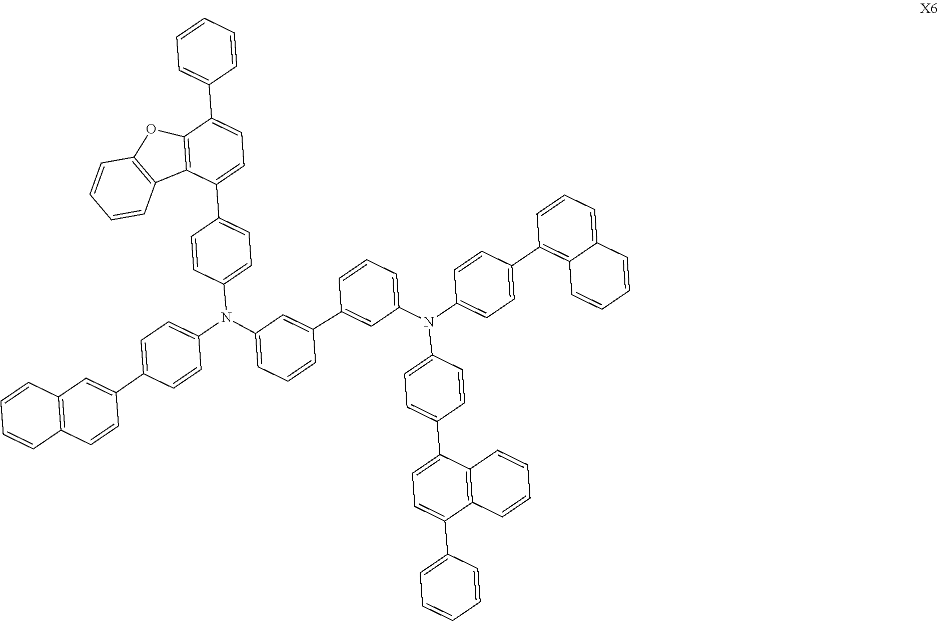 [SMILES: c1ccc(-c2ccc(-c3ccc(N(c4ccc(-c5cccc6ccccc56)cc4)c4cccc(-c5cccc(N(c6ccc(-c7ccc8ccccc8c7)cc6)c6ccc(-c7ccc(-c8ccccc8)c8oc9ccccc9c78)cc6)c5)c4)cc3)c3ccccc23)cc1]

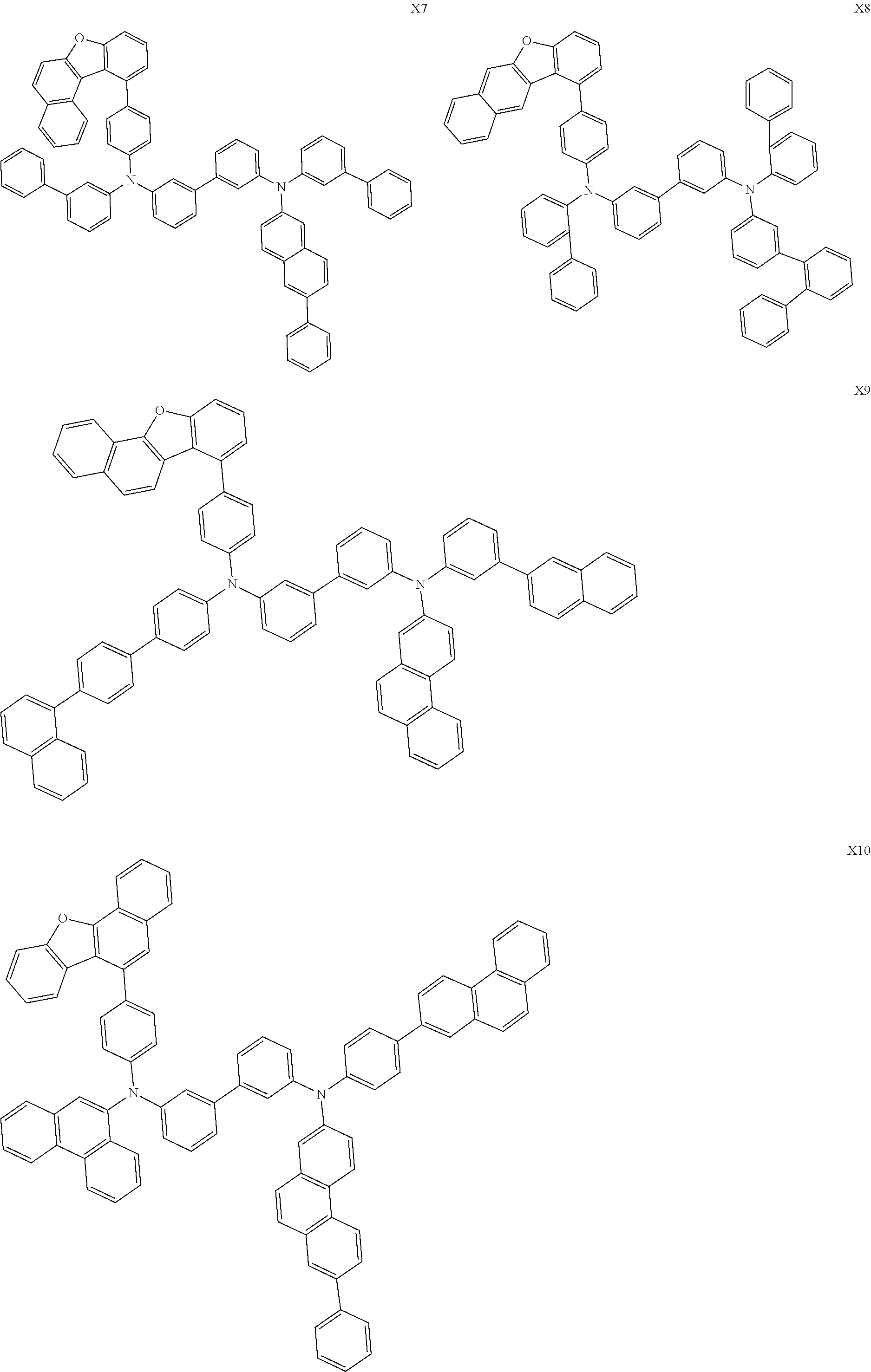

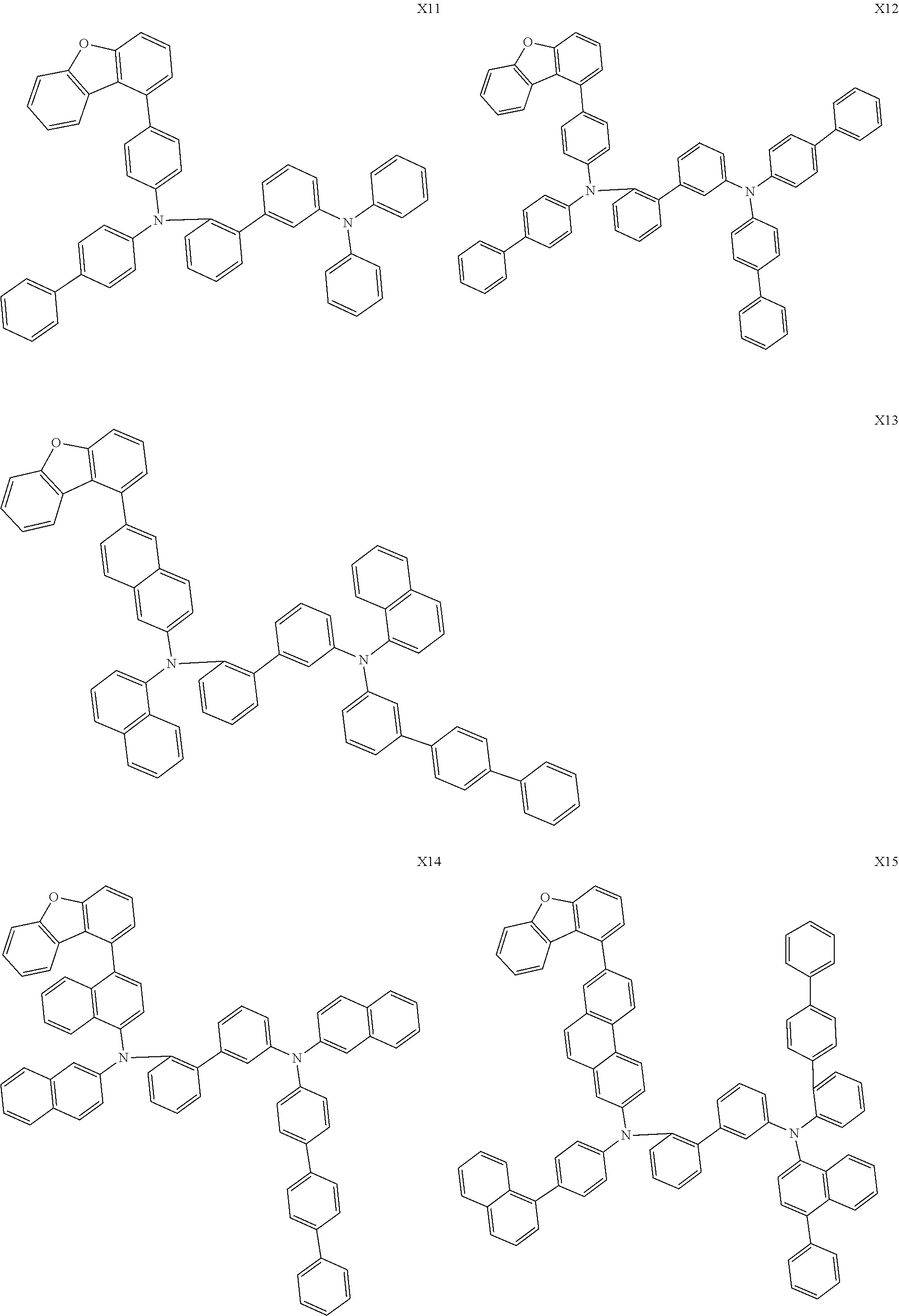

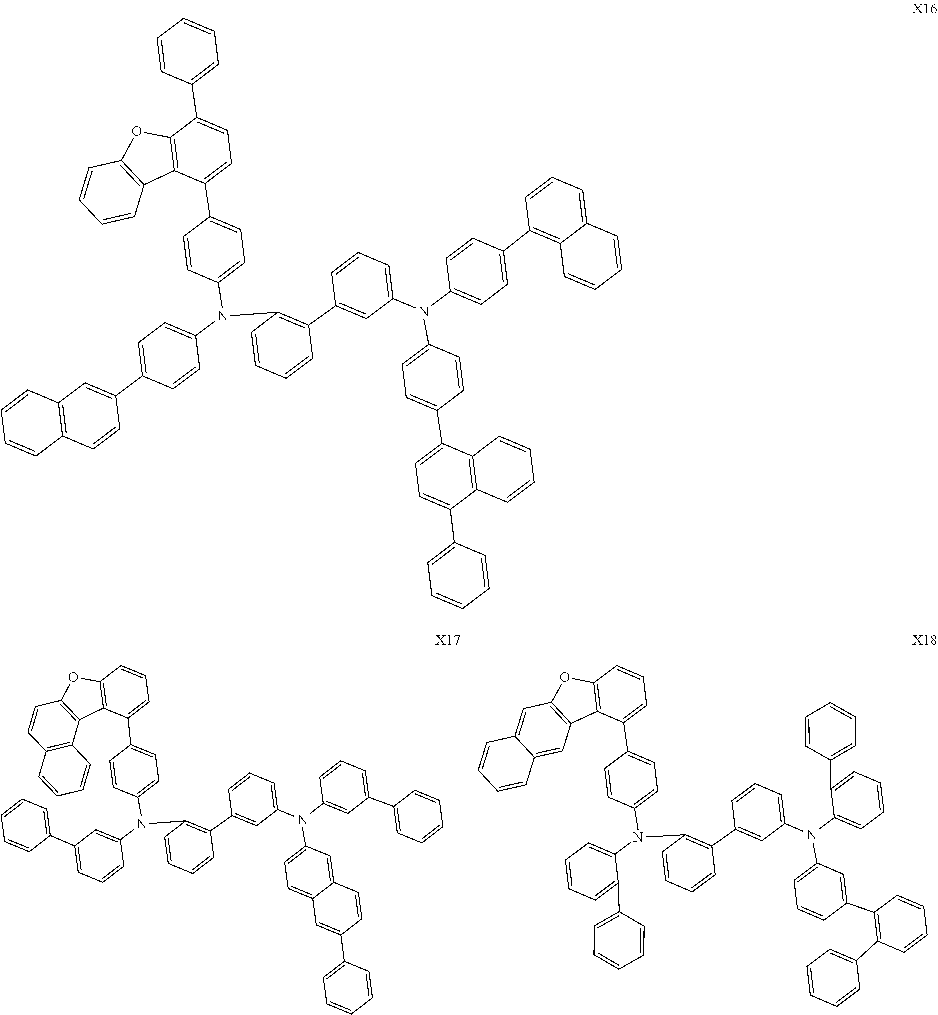 [SMILES: c1ccc(-c2ccc(-c3ccc(N(c4ccc(-c5cccc6ccccc56)cc4)c4cccc(-c5ccccc5N(c5ccc(-c6ccc7ccccc7c6)cc5)c5ccc(-c6ccc(-c7ccccc7)c7oc8ccccc8c67)cc5)c4)cc3)c3ccccc23)cc1 ; c1ccc(-c2cccc(N(c3cccc(-c4ccccc4N(c4ccc(-c5cccc6oc7ccc8ccccc8c7c56)cc4)c4cccc(-c5ccccc5)c4)c3)c3ccc4cc(-c5ccccc5)ccc4c3)c2)cc1 ; c1ccc(-c2ccccc2-c2cccc(N(c3cccc(-c4ccccc4N(c4ccc(-c5cccc6oc7cc8ccccc8cc7c56)cc4)c4ccccc4-c4ccccc4)c3)c3ccccc3-c3ccccc3)c2)cc1]

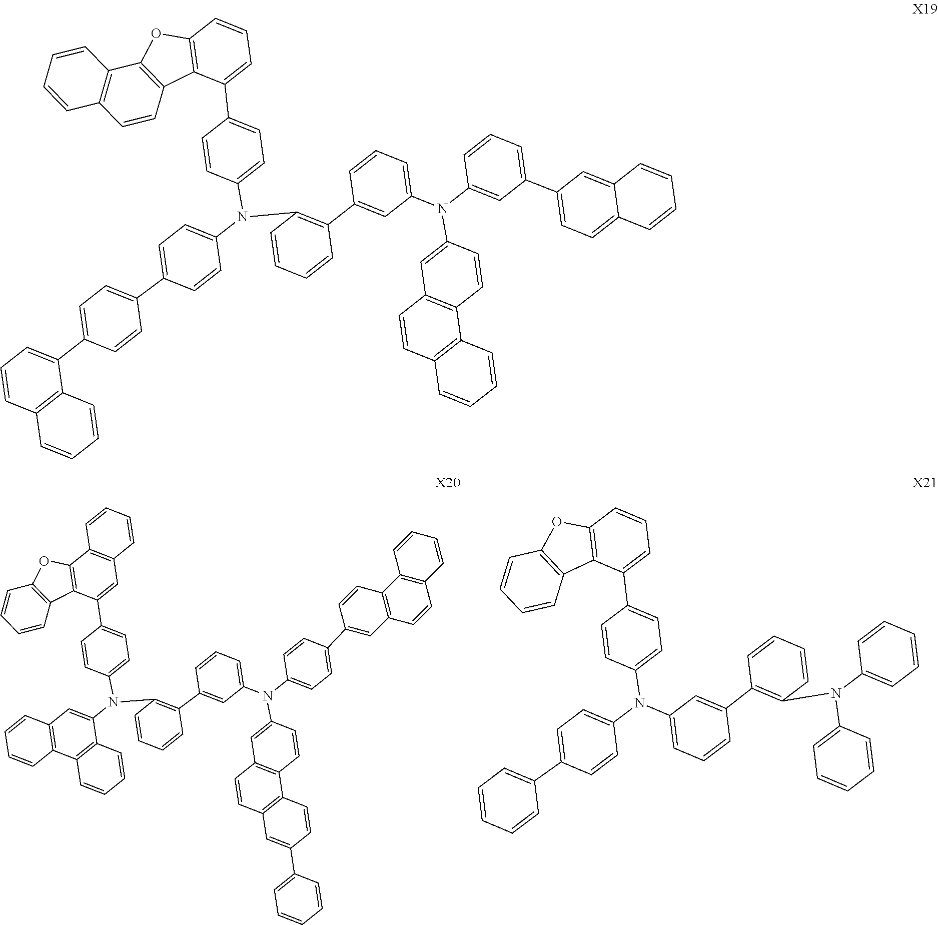 [SMILES: c1cc(-c2ccc3ccccc3c2)cc(N(c2cccc(-c3ccccc3N(c3ccc(-c4ccc(-c5cccc6ccccc56)cc4)cc3)c3ccc(-c4cccc5oc6c7ccccc7ccc6c45)cc3)c2)c2ccc3c(ccc4ccccc43)c2)c1 ; c1ccc(-c2ccc3c(ccc4cc(N(c5ccc(-c6ccc7c(ccc8ccccc87)c6)cc5)c5cccc(-c6ccccc6N(c6ccc(-c7cc8ccccc8c8oc9ccccc9c78)cc6)c6cc7ccccc7c7ccccc67)c5)ccc43)c2)cc1 ; c1ccc(-c2ccc(N(c3ccc(-c4cccc5oc6ccccc6c45)cc3)c3cccc(-c4ccccc4N(c4ccccc4)c4ccccc4)c3)cc2)cc1]

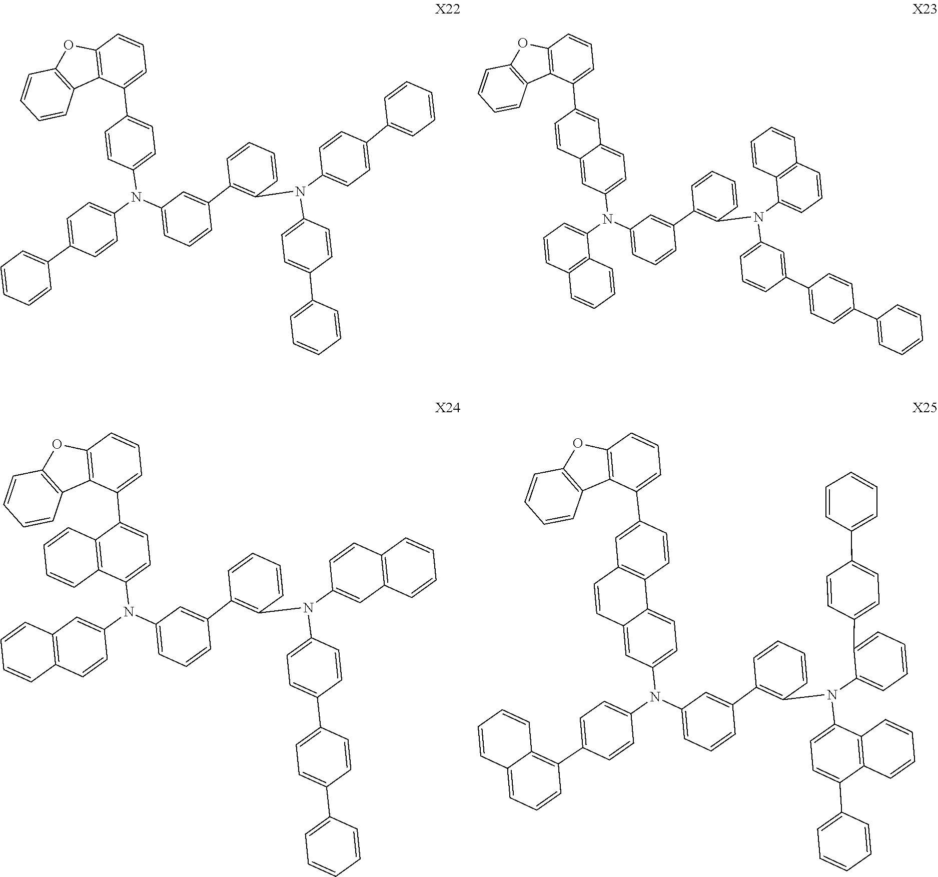

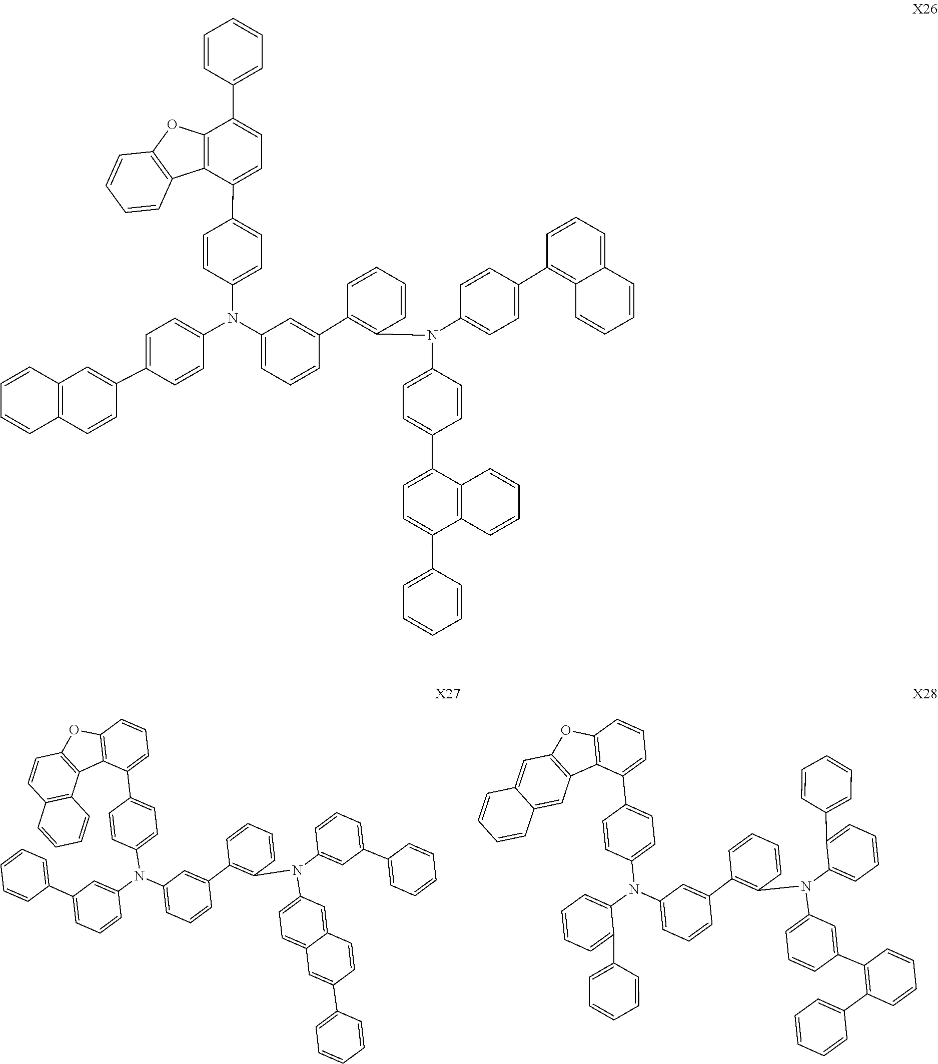 [SMILES: c1ccc(-c2ccc(-c3ccc(N(c4ccc(-c5cccc6ccccc56)cc4)c4ccccc4-c4cccc(N(c5ccc(-c6ccc7ccccc7c6)cc5)c5ccc(-c6ccc(-c7ccccc7)c7oc8ccccc8c67)cc5)c4)cc3)c3ccccc23)cc1 ; c1ccc(-c2cccc(N(c3ccc(-c4cccc5oc6ccc7ccccc7c6c45)cc3)c3cccc(-c4ccccc4N(c4cccc(-c5ccccc5)c4)c4ccc5cc(-c6ccccc6)ccc5c4)c3)c2)cc1 ; c1ccc(-c2ccccc2-c2cccc(N(c3ccccc3-c3ccccc3)c3ccccc3-c3cccc(N(c4ccc(-c5cccc6oc7cc8ccccc8cc7c56)cc4)c4ccccc4-c4ccccc4)c3)c2)cc1]

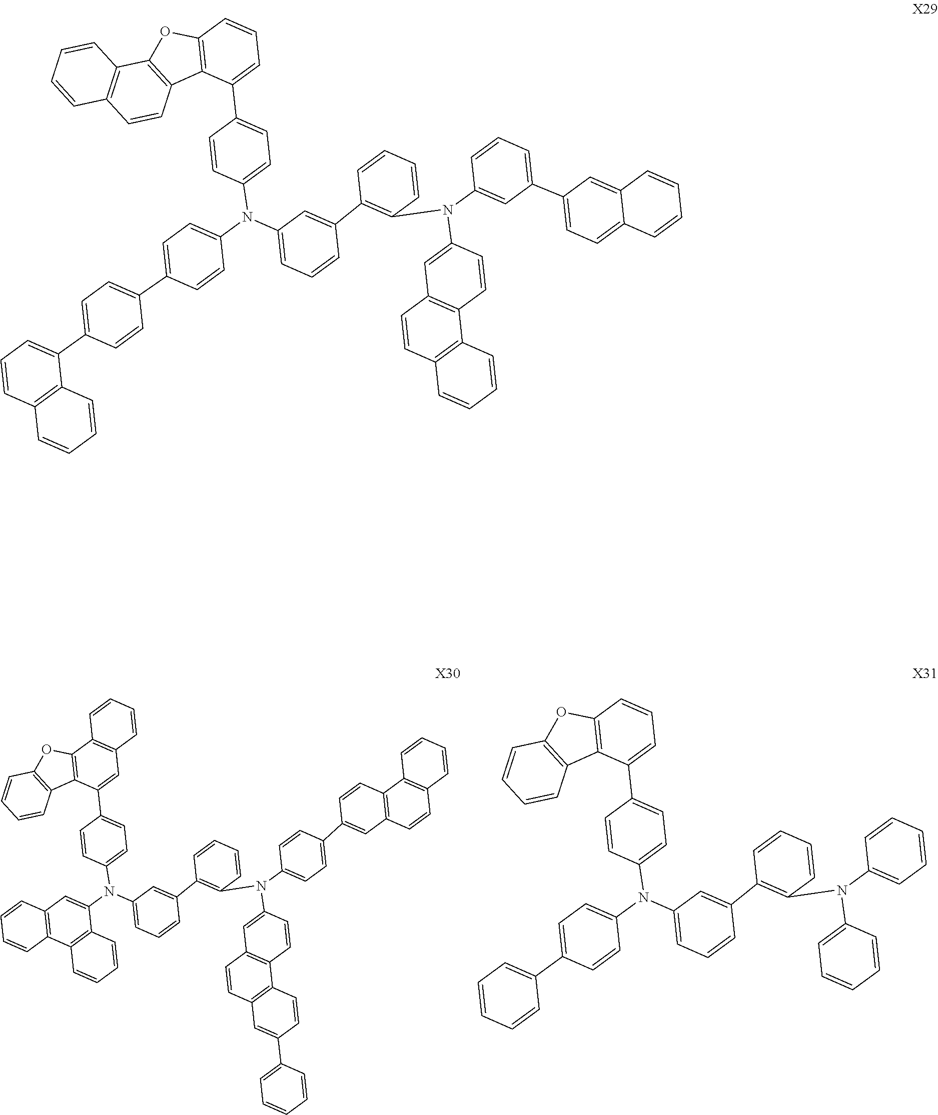 [SMILES: c1cc(-c2ccccc2N(c2cccc(-c3ccc4ccccc4c3)c2)c2ccc3c(ccc4ccccc43)c2)cc(N(c2ccc(-c3ccc(-c4cccc5ccccc45)cc3)cc2)c2ccc(-c3cccc4oc5c6ccccc6ccc5c34)cc2)c1 ; c1ccc(-c2ccc3c(ccc4cc(N(c5ccc(-c6ccc7c(ccc8ccccc87)c6)cc5)c5ccccc5-c5cccc(N(c6ccc(-c7cc8ccccc8c8oc9ccccc9c78)cc6)c6cc7ccccc7c7ccccc67)c5)ccc43)c2)cc1 ; c1ccc(-c2ccc(N(c3ccc(-c4cccc5oc6ccccc6c45)cc3)c3cccc(-c4ccccc4N(c4ccccc4)c4ccccc4)c3)cc2)cc1]

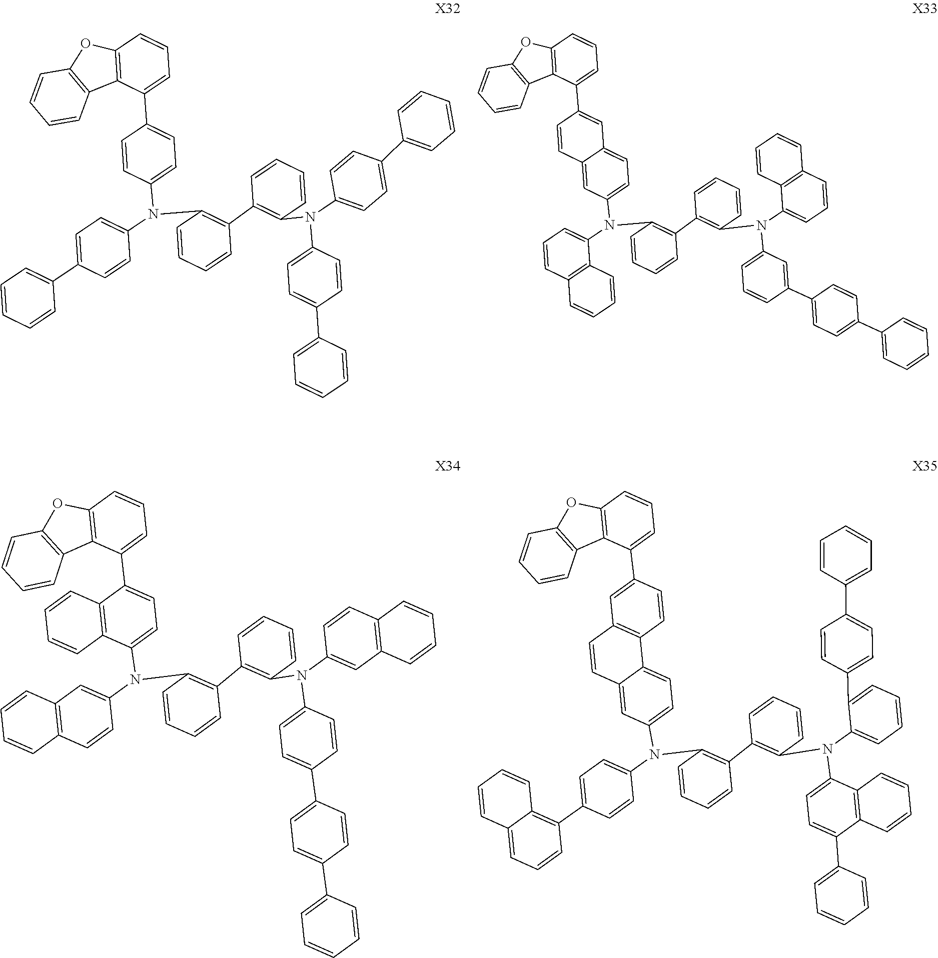

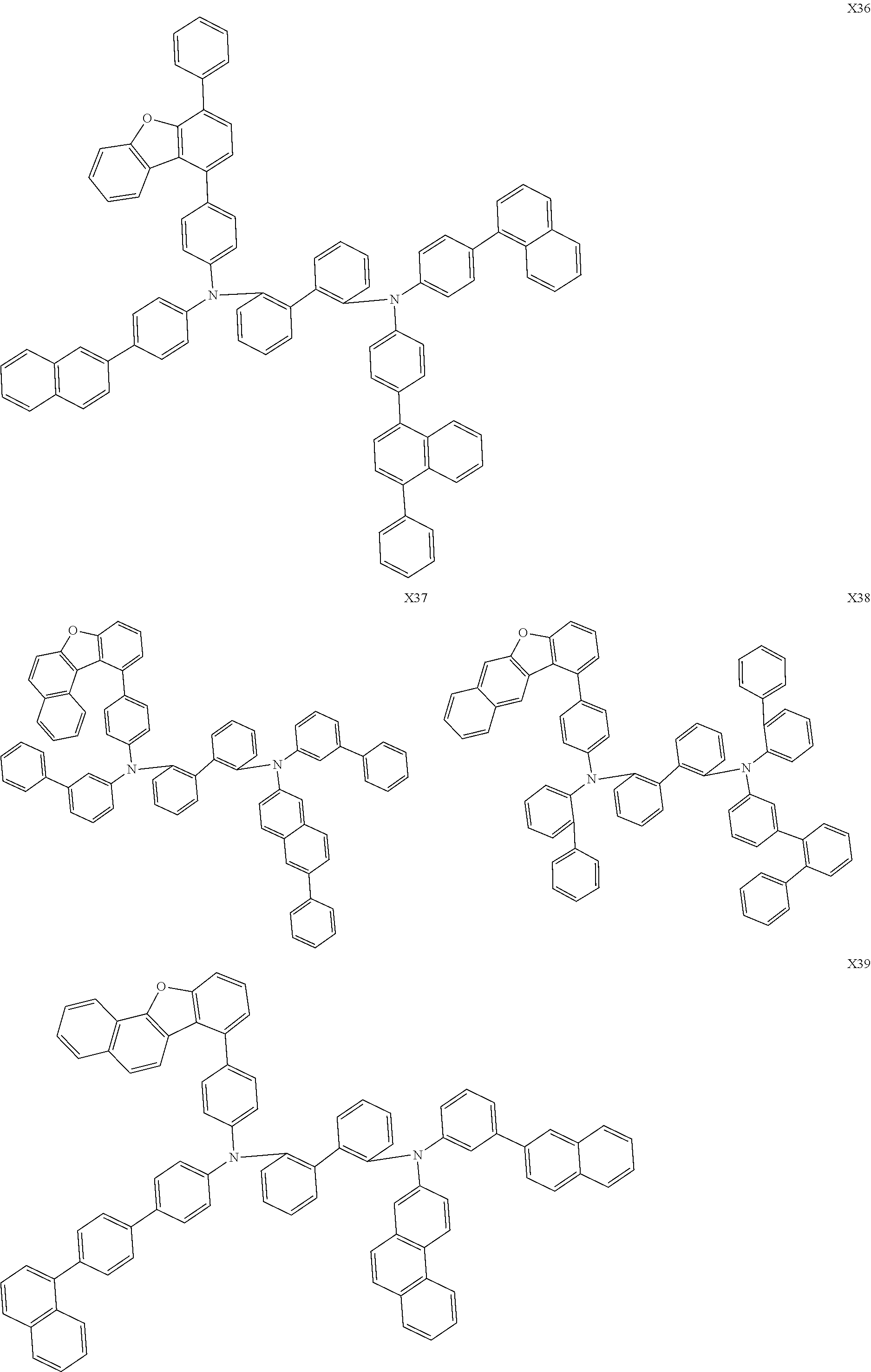

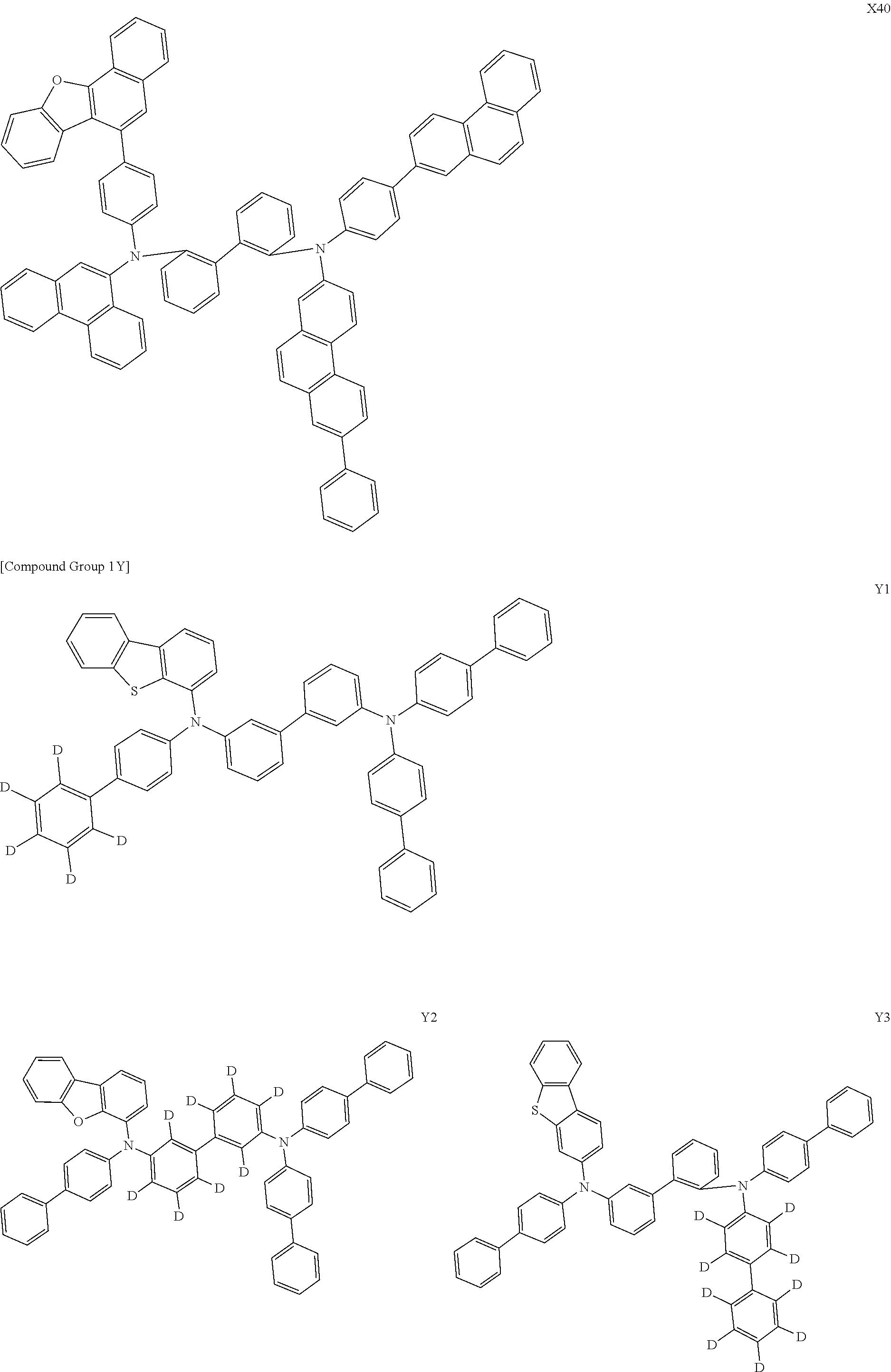

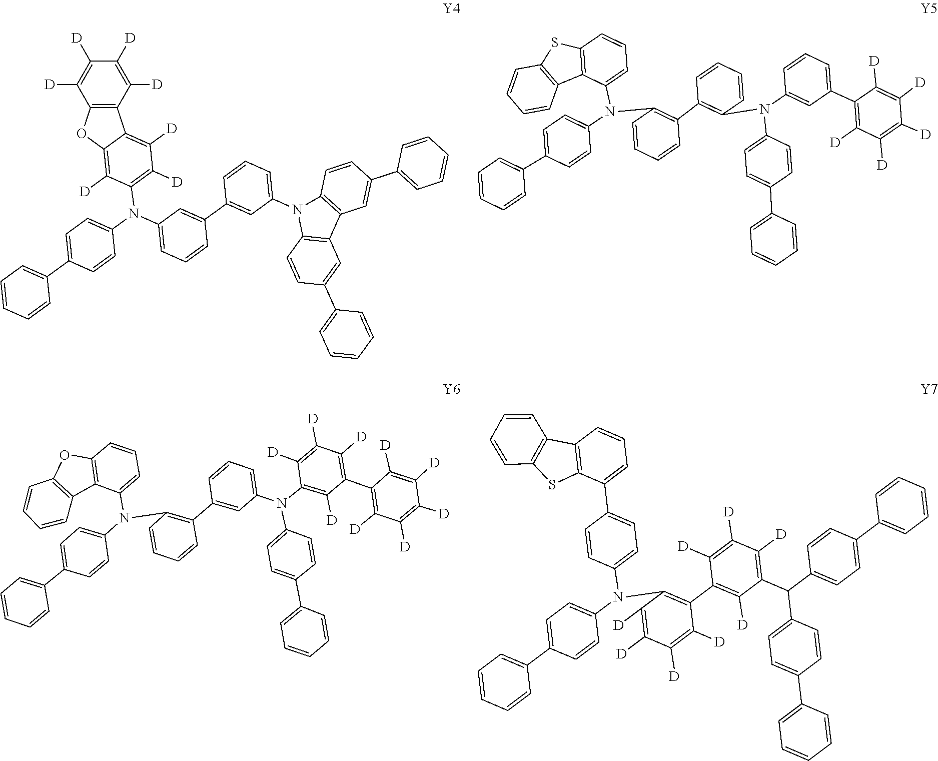

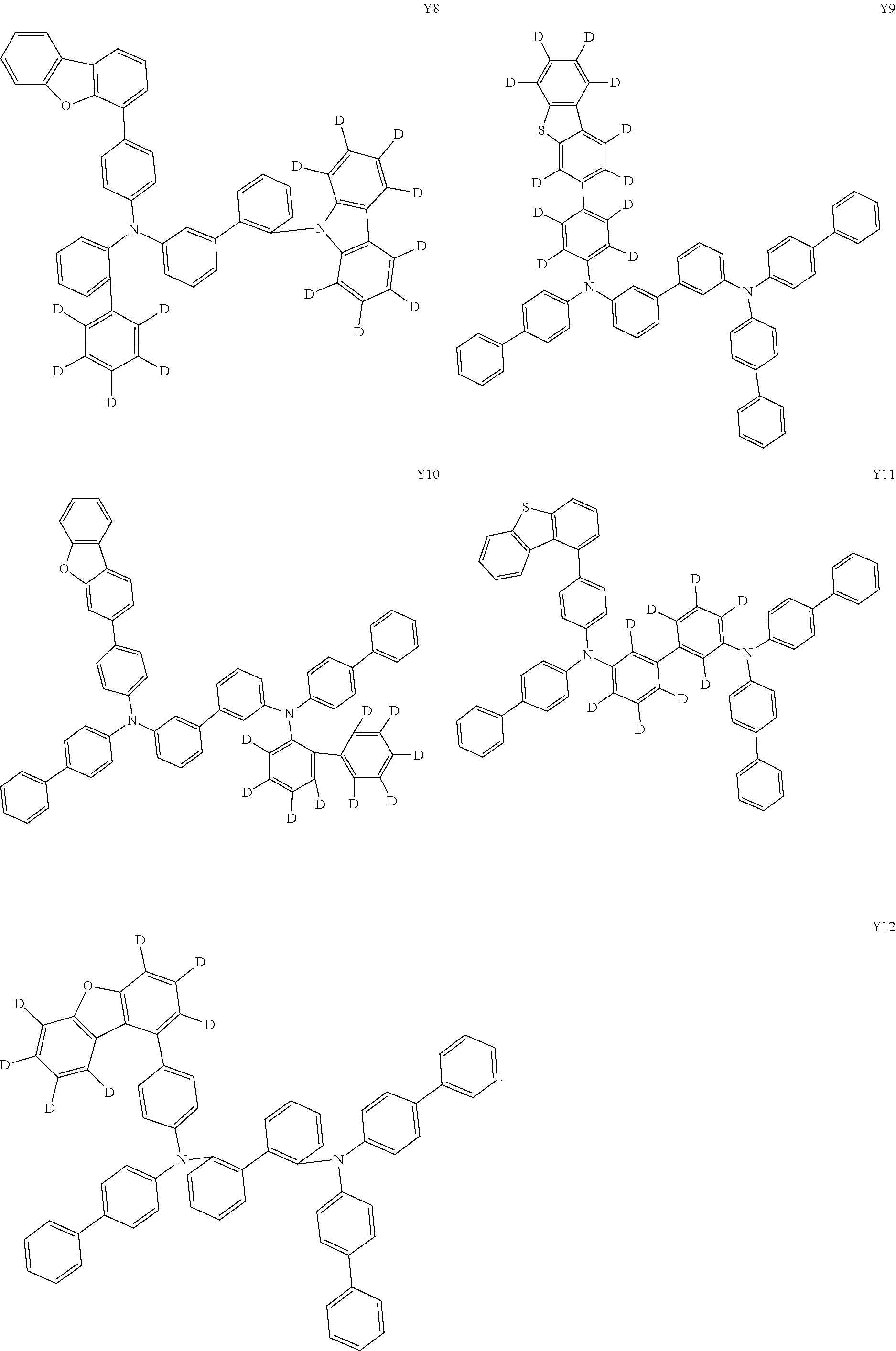

14. An amine compound represented by Formula 1: 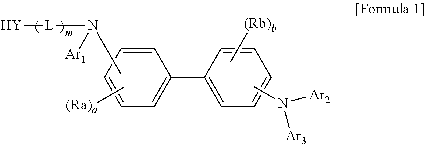
wherein in Formula 1,
a and b are each independently an integer from 0 to 4,
Ra and Rb are each independently a hydrogen atom or a deuterium atom,
m is 0 or 1,
L is a substituted or unsubstituted arylene group of 6 to 40 ring-forming carbon atoms, and
HT is a group represented by Formula 2: 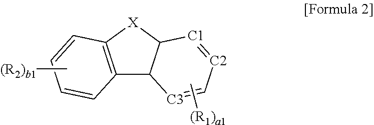
wherein in Formula 2,
C1 to C3 each represent a carbon atom in an aromatic ring,
a position selected from C1 to C3 is bonded to Formula 1,
X is O or S,
a1 is an integer from 0 to 3,
b1 is an integer from 0 to 4, and
R₁ and R₂ are each independently a hydrogen atom, a deuterium atom, a substituted or unsubstituted alkyl group of 1 to 20 carbon atoms, a substituted or unsubstituted alkenyl group of 2 to 20 carbon atoms, or a substituted or unsubstituted aryl group of 6 to 40 ring-forming carbon atoms, or multiple R₁ groups or multiple R₂ groups are combined with each other to form an aromatic ring,
wherein in Formula 1,
in case where m is 1, Ar₁ to Ar₃ are each independently a substituted or unsubstituted aryl group of 6 to 40 ring-forming carbon atoms,
in case where m is 0,
Ar₁ to Ar₃ are each independently a substituted or unsubstituted aryl group of 6 to 40 ring-forming carbon atoms, or a substituted or unsubstituted heteroaryl group of 5 to 40 ring-forming carbon atoms, or
Ar₁ is a substituted or unsubstituted aryl group of 6 to 40 ring-forming carbon atoms, or a substituted or unsubstituted heteroaryl group of 5 to 40 ring-forming carbon atoms, and Ar₂ and Ar₃ are combined with an adjacent group to form a carbazole derivative group except for groups represented by CZ-1 to CZ-3,
in case where m is 0, none of Ar₁ to Ar₃ are a substituted or unsubstituted fluorenyl group, a substituted or unsubstituted pyrenyl group, an unsubstituted carbazolyl group, a 2-dibenzofuranyl group, and a 2-dibenzothiophenyl group, and
in case where m is 0, X is not O, and Ar₁ is not an unsubstituted phenyl group, and wherein in CZ-1 to CZ-3, [symbol]
represents a binding site to a neighboring atom:

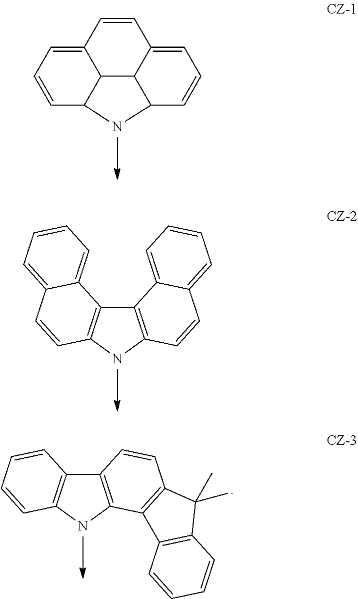 [SMILES: C1=CC2NC3C=CC=C4C=CC(=C1)C2C43 ; c1ccc2c(c1)ccc1[nH]c3ccc4ccccc4c3c12 ; CC1(C)c2ccccc2-c2c1ccc1c2[nH]c2ccccc21]

15. The amine compound of claim 14, wherein Formula 1 is represented by one of Formula 1-1 to Formula 1-4: 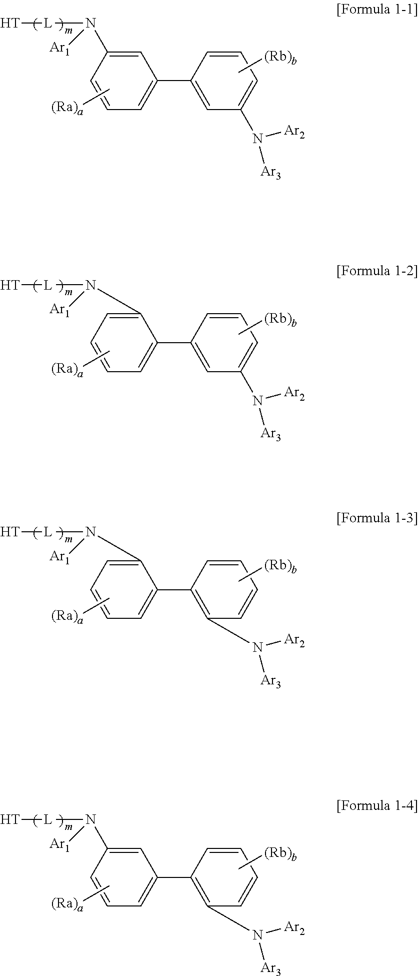
wherein in Formula 1-1 to Formula 1-4,
a, b, Ra, Rb, m, L, Ar₁, Ar₂, Ar₃, and HT are the same as defined in connection with Formula 1 and Formula 2.

16. The amine compound of claim 14, wherein Formula 1 is represented by the following Formula 1-A or Formula 1-B: 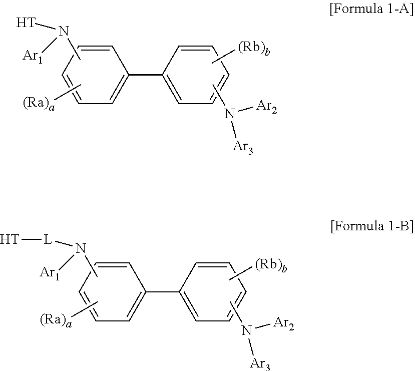
wherein in Formula 1-A an Formula 1-B,
a, b, Ra, Rb, L, Ar₁, Ar₂, Ar₃, and HT are the same as defined in connection with Formula 1 and Formula 2.

17. The amine compound of claim 16, wherein in Formula 1-B,
L is represented by one of L-1 to L-3: 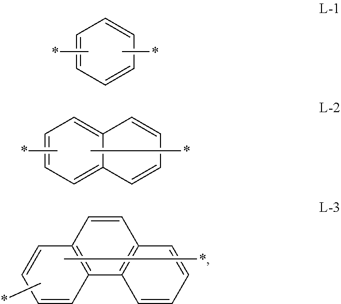
wherein in L-1 to L-3, [symbol]
represents a binding site to a neighboring atom.

18. The amine compound of claim 14, wherein Formula 2 is represented by one of Formula 2-1 to Formula 2-3: 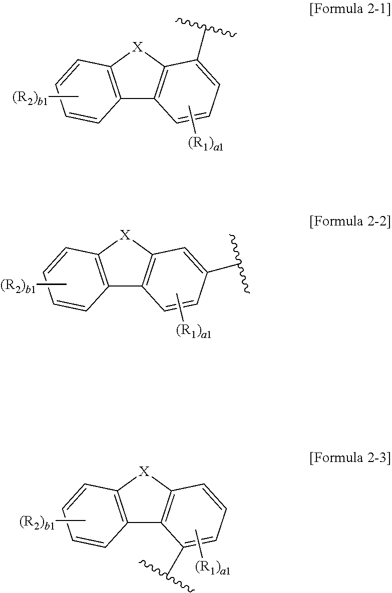
wherein in Formula 2-1 to Formula 2-3,
X, a1, b1, R₁, and R₂ are the same as defined in connection with Formula 2, and 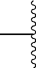
represents a binding site to a neighboring atom.

19. The amine compound of claim 14, wherein Formula 2 is represented by one of Formula 2-a to Formula 2-e: 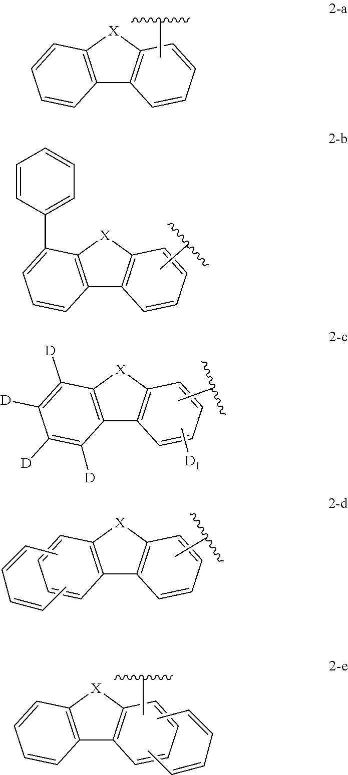
wherein in Formula 2-a to Formula 2-e,
X is the same as defined in connection with Formula 2, and 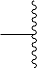
represents a binding site to Formula 1 at a position selected from C1 to C3 in Formula 2.

20. The amine compound of claim 14, wherein
at least one of Ra, Rb, R₁, and R₂ is a deuterium atom, or
at least one of Ar₁ to Ar₃ is an aryl group of 6 to 40 ring-forming carbon atoms substituted with at least one deuterium atom.

21. The amine compound of claim 14, wherein
a and b are each 4, and
Ra and Rb are each deuterium atoms.

22. The amine compound of claim 14, wherein,
in case where m is 0, and at least one of Ar₁ to Ar₃ is a substituted or unsubstituted heteroaryl group of 5 to 40 ring-forming carbon atoms,
the heteroaryl group comprises O or S as a heteroatom.

23. The amine compound of claim 14, wherein Formula 1 is one selected from Compound Group 1A to Compound Group 1Y: 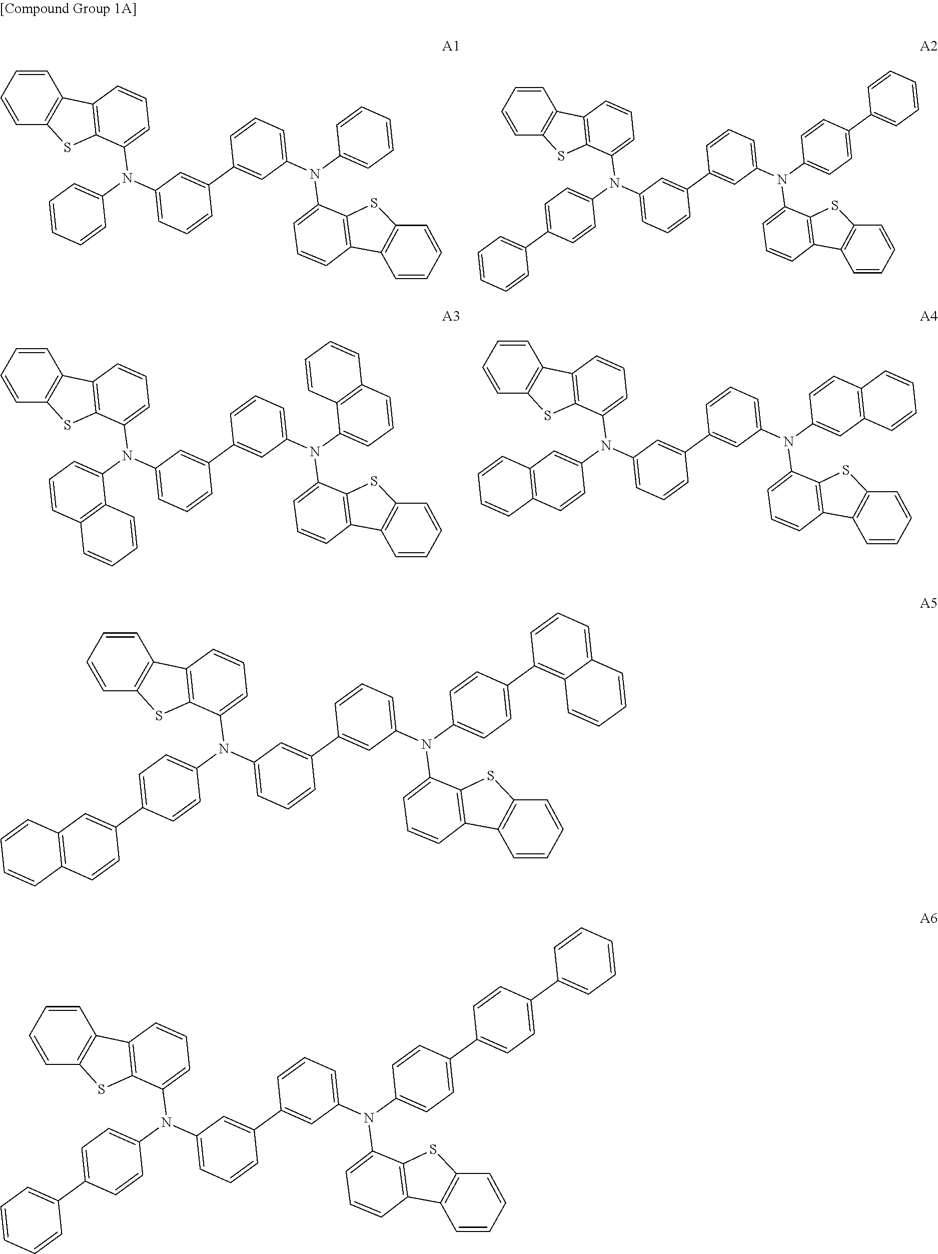 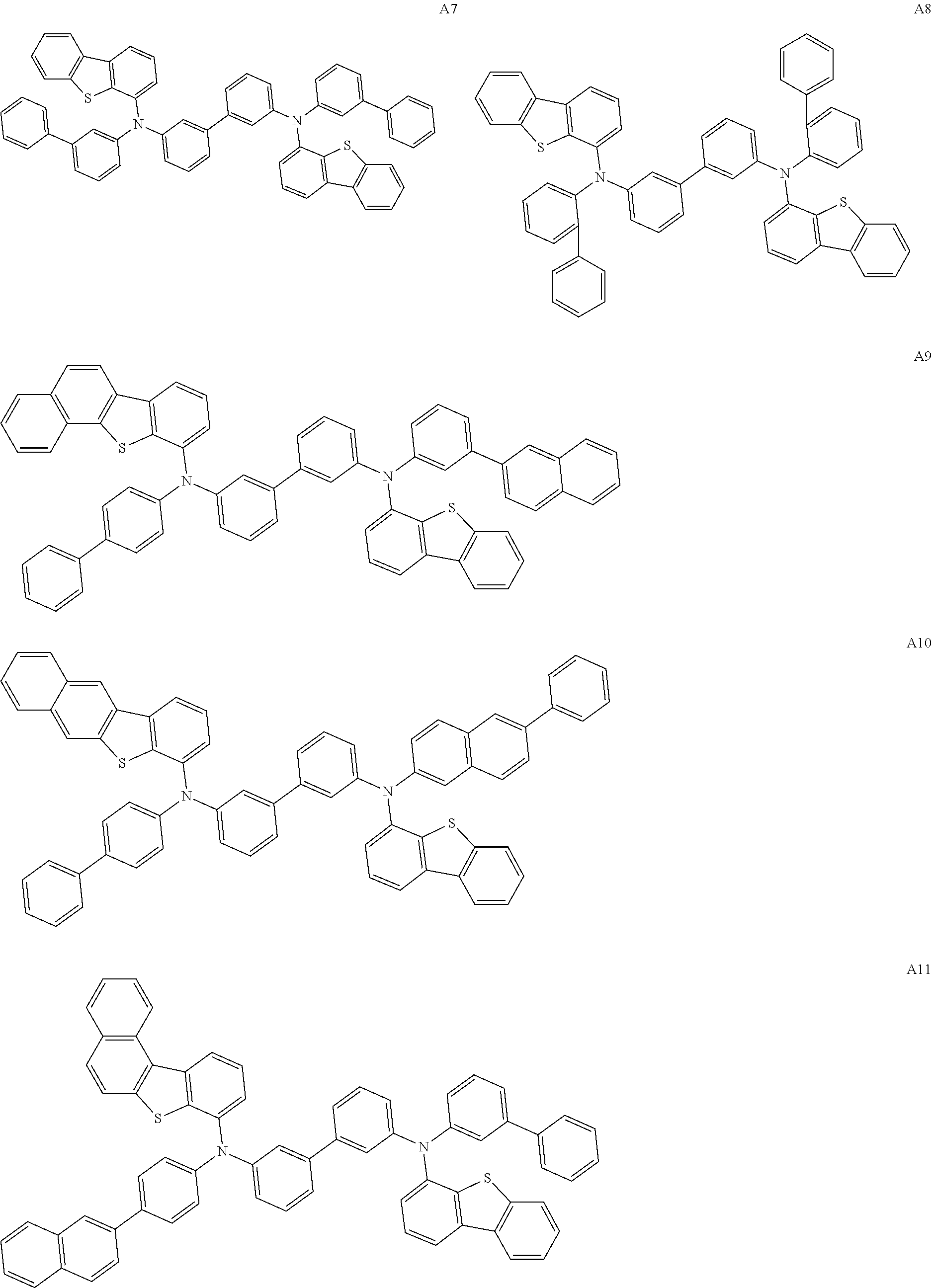 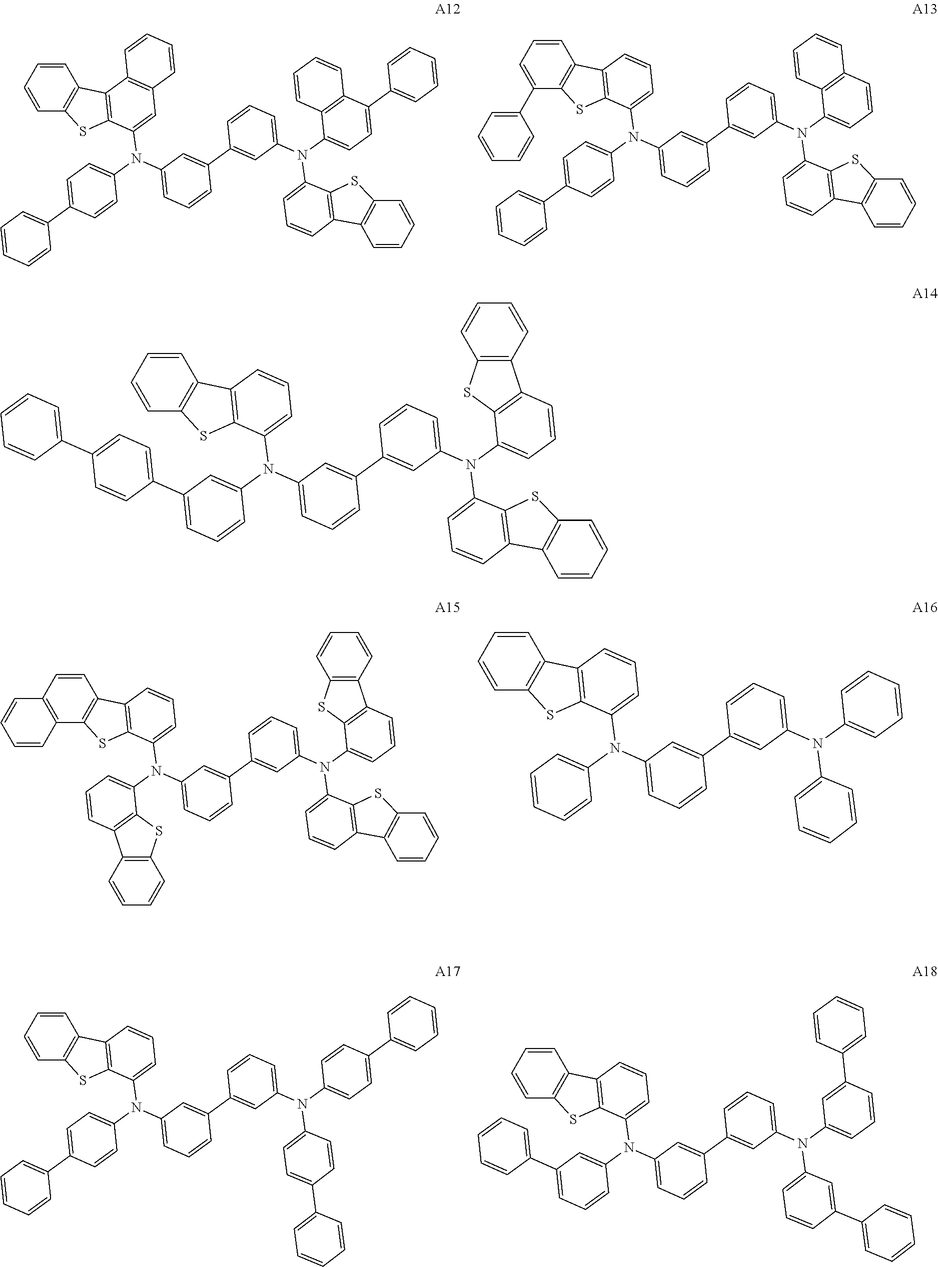 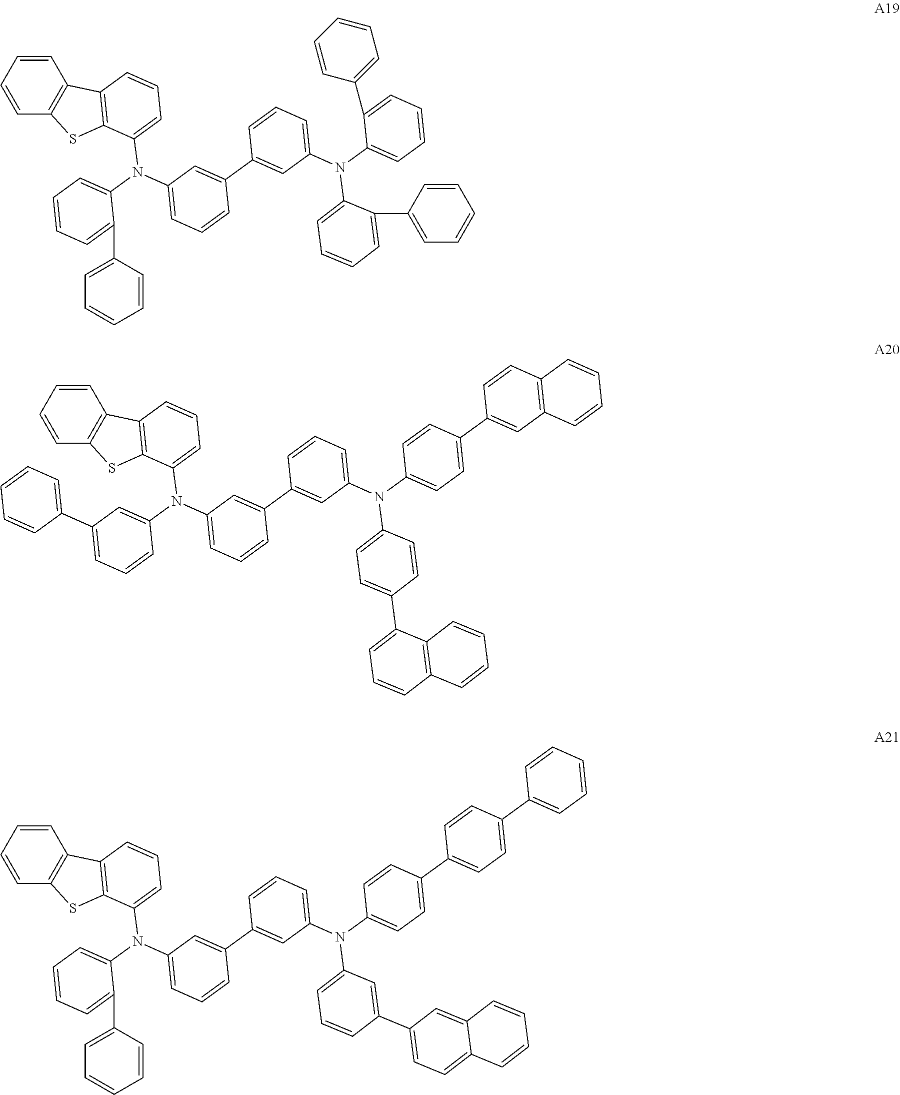 [SMILES: c1ccc(-c2ccccc2N(c2cccc(-c3cccc(N(c4ccccc4-c4ccccc4)c4cccc5c4sc4ccccc45)c3)c2)c2ccccc2-c2ccccc2)cc1 ; c1ccc(-c2cccc(N(c3cccc(-c4cccc(N(c5ccc(-c6ccc7ccccc7c6)cc5)c5ccc(-c6cccc7ccccc67)cc5)c4)c3)c3cccc4c3sc3ccccc34)c2)cc1 ; c1ccc(-c2ccc(-c3ccc(N(c4cccc(-c5cccc(N(c6ccccc6-c6ccccc6)c6cccc7c6sc6ccccc67)c5)c4)c4cccc(-c5ccc6ccccc6c5)c4)cc3)cc2)cc1] 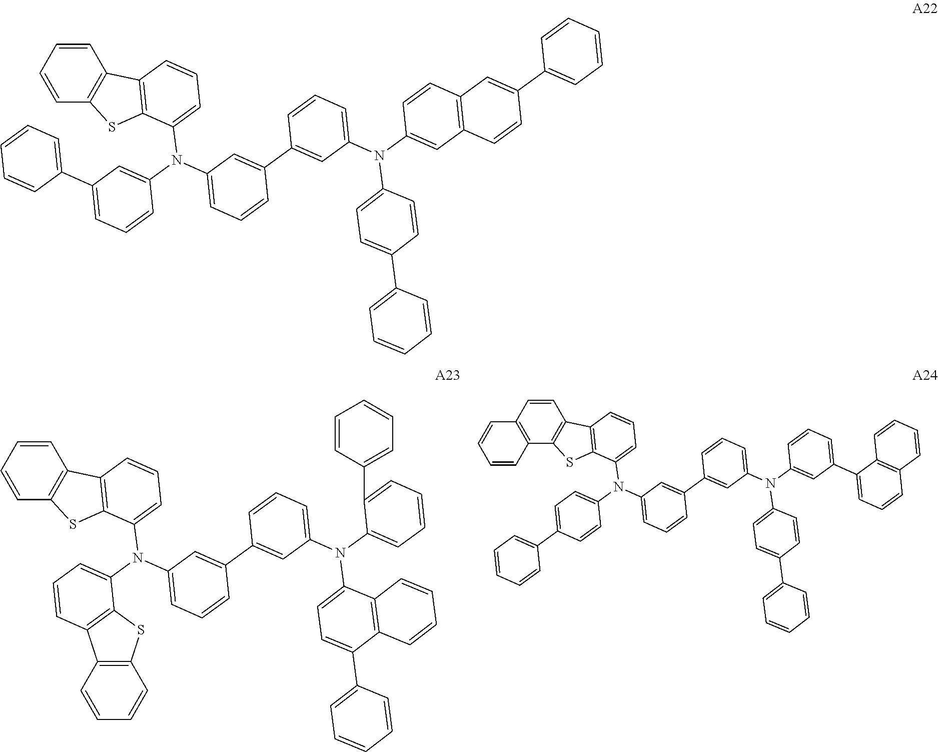 [SMILES: c1ccc(-c2ccc(N(c3cccc(-c4cccc(N(c5cccc(-c6ccccc6)c5)c5cccc6c5sc5ccccc56)c4)c3)c3ccc4cc(-c5ccccc5)ccc4c3)cc2)cc1 ; c1ccc(-c2ccccc2N(c2cccc(-c3cccc(N(c4cccc5c4sc4ccccc45)c4cccc5c4sc4ccccc45)c3)c2)c2ccc(-c3ccccc3)c3ccccc23)cc1 ; c1ccc(-c2ccc(N(c3cccc(-c4cccc(N(c5ccc(-c6ccccc6)cc5)c5cccc6c5sc5c7ccccc7ccc65)c4)c3)c3cccc(-c4cccc5ccccc45)c3)cc2)cc1] 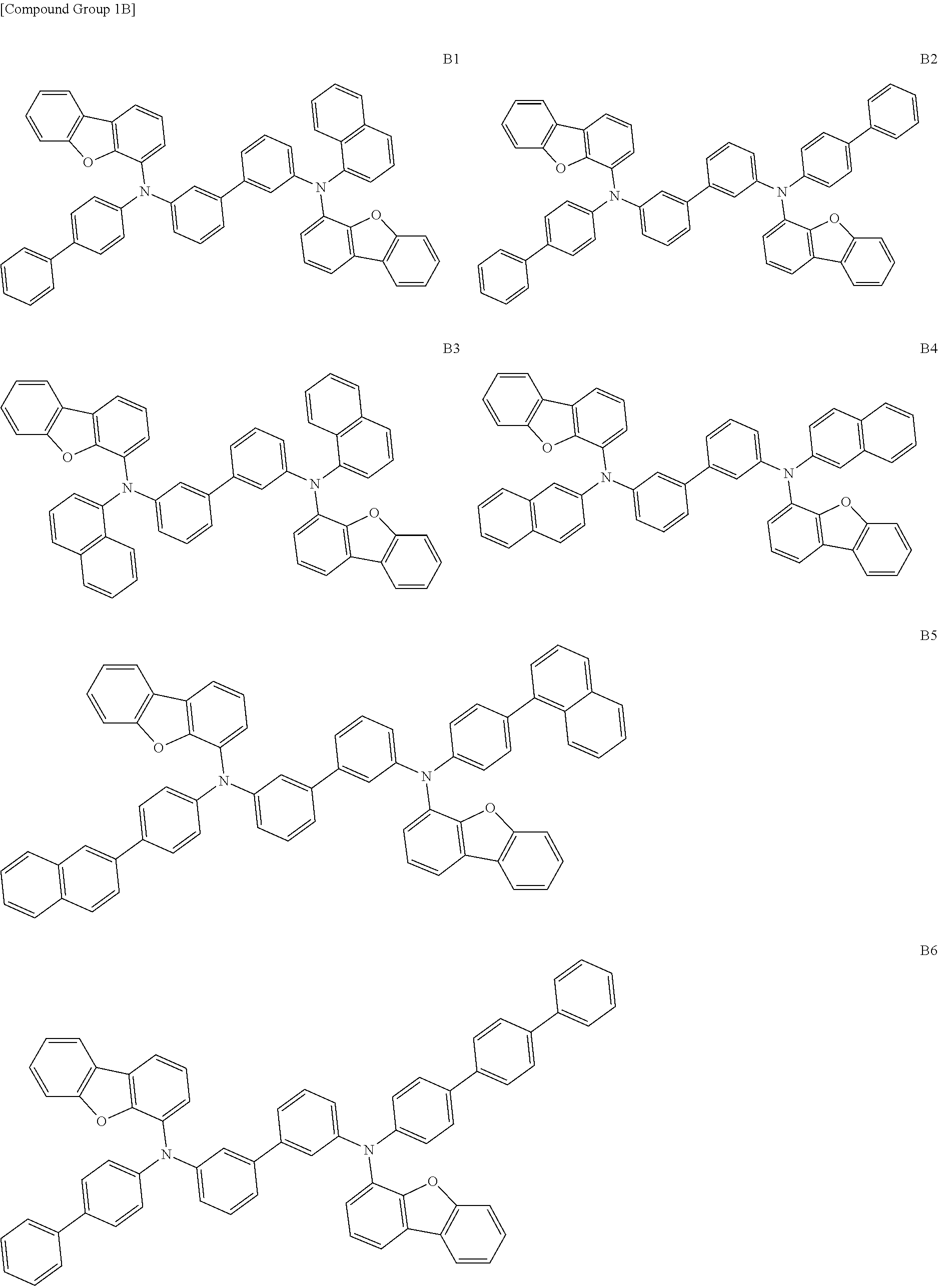 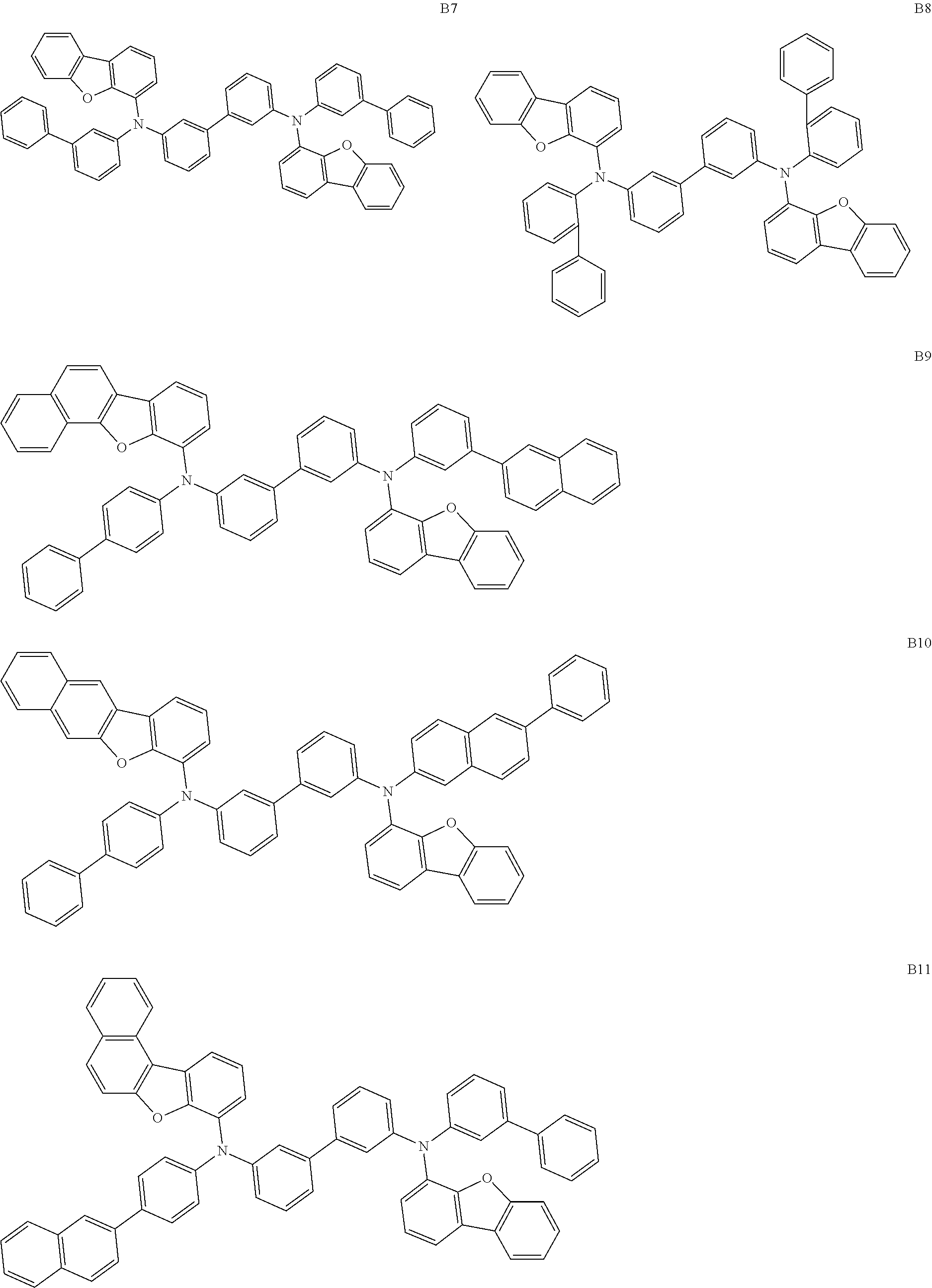 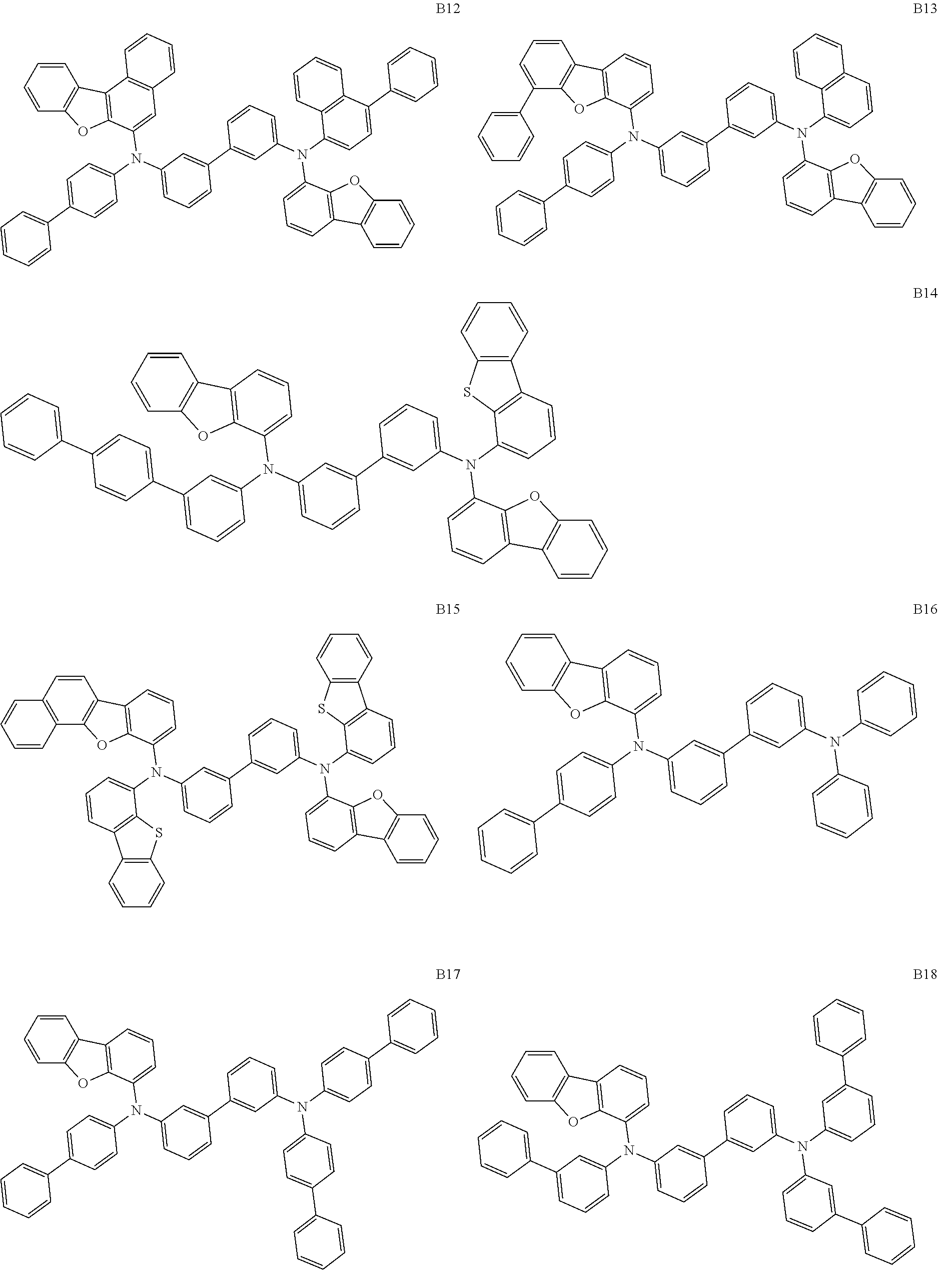 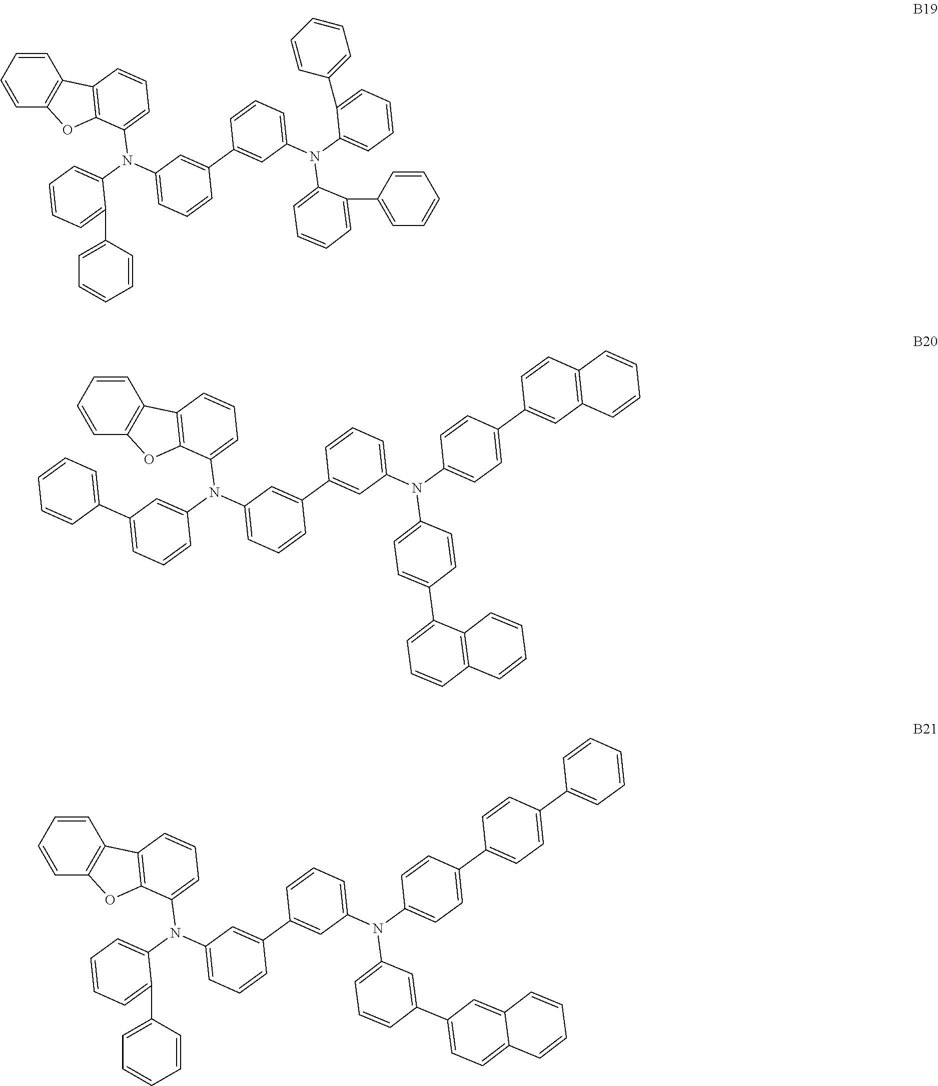 [SMILES: c1ccc(-c2ccccc2N(c2cccc(-c3cccc(N(c4ccccc4-c4ccccc4)c4cccc5c4oc4ccccc45)c3)c2)c2ccccc2-c2ccccc2)cc1 ; c1ccc(-c2cccc(N(c3cccc(-c4cccc(N(c5ccc(-c6ccc7ccccc7c6)cc5)c5ccc(-c6cccc7ccccc67)cc5)c4)c3)c3cccc4c3oc3ccccc34)c2)cc1 ; c1ccc(-c2ccc(-c3ccc(N(c4cccc(-c5cccc(N(c6ccccc6-c6ccccc6)c6cccc7c6oc6ccccc67)c5)c4)c4cccc(-c5ccc6ccccc6c5)c4)cc3)cc2)cc1] 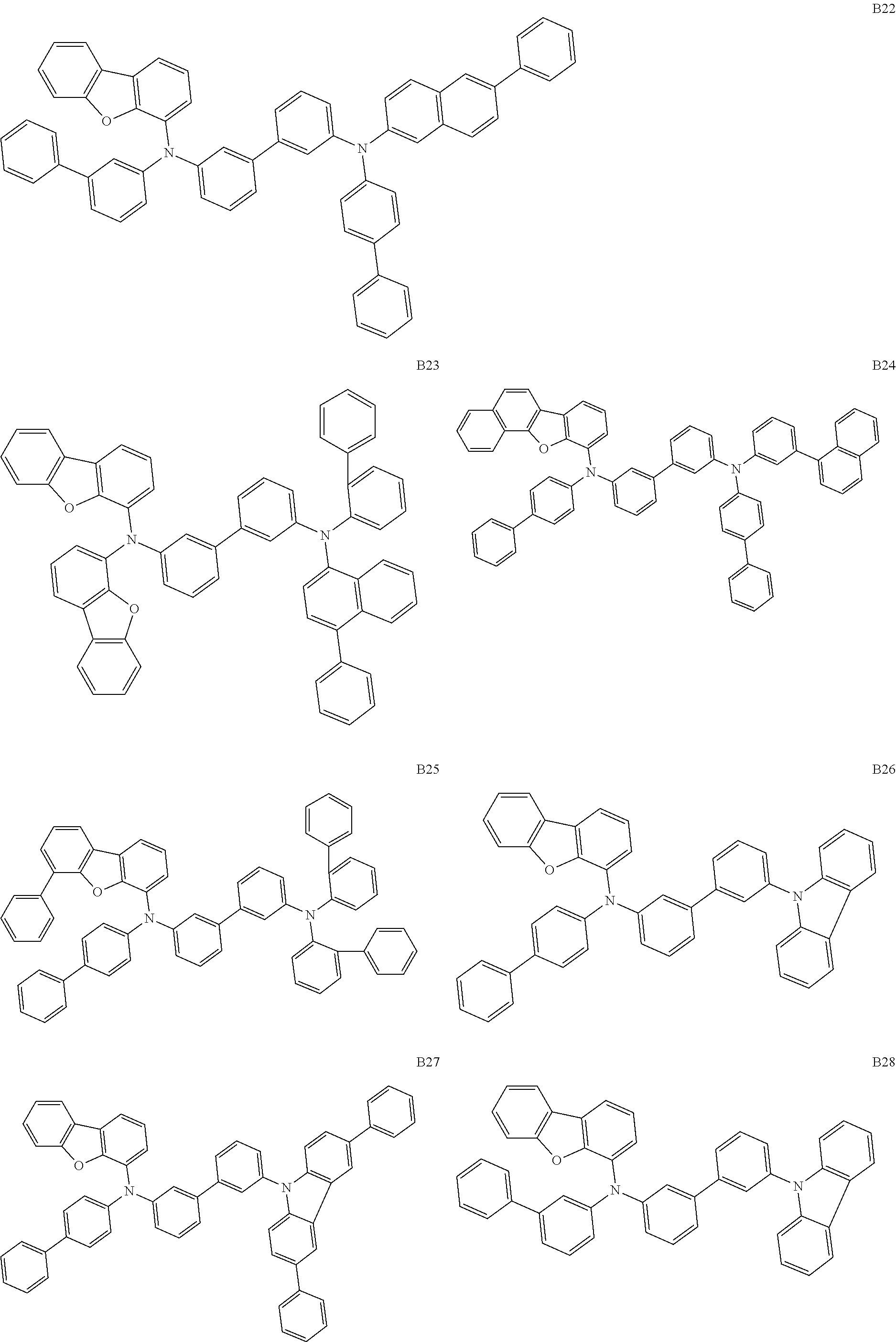 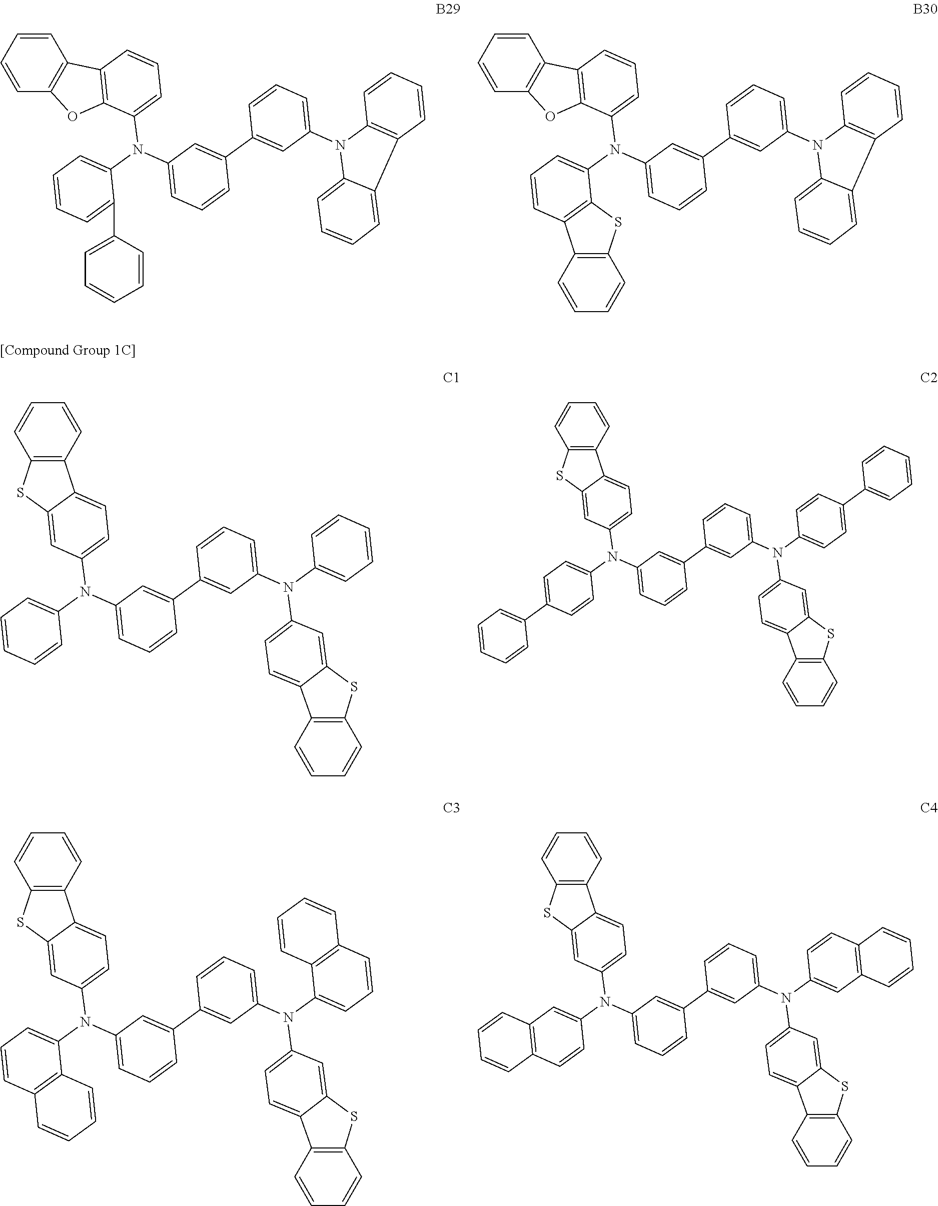 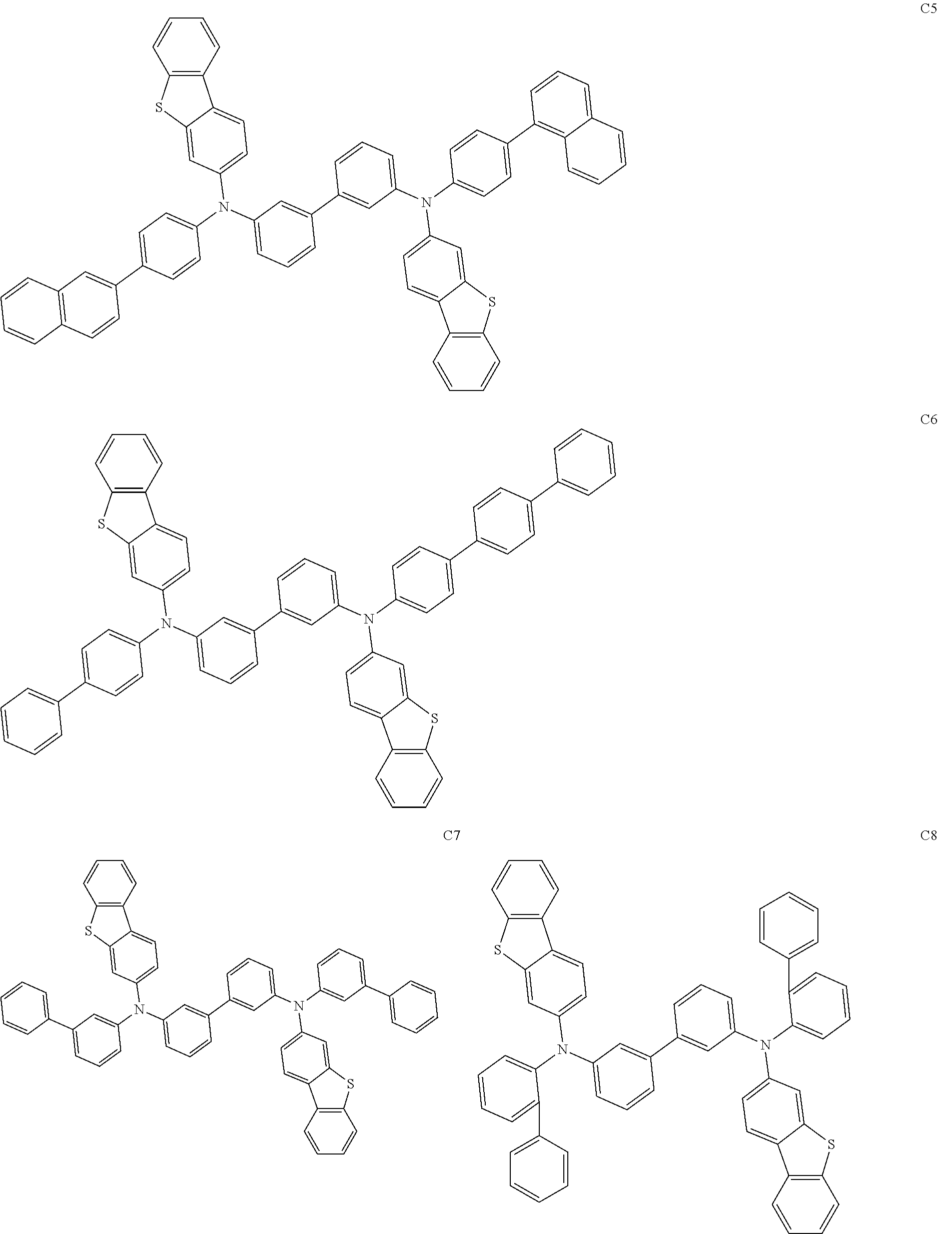 [SMILES: c1cc(-c2cccc(N(c3ccc(-c4cccc5ccccc45)cc3)c3ccc4c(c3)sc3ccccc34)c2)cc(N(c2ccc(-c3ccc4ccccc4c3)cc2)c2ccc3c(c2)sc2ccccc23)c1 ; c1ccc(-c2ccc(-c3ccc(N(c4cccc(-c5cccc(N(c6ccc(-c7ccccc7)cc6)c6ccc7c(c6)sc6ccccc67)c5)c4)c4ccc5c(c4)sc4ccccc45)cc3)cc2)cc1 ; c1ccc(-c2cccc(N(c3cccc(-c4cccc(N(c5cccc(-c6ccccc6)c5)c5ccc6c(c5)sc5ccccc56)c4)c3)c3ccc4c(c3)sc3ccccc34)c2)cc1 ; c1ccc(-c2ccccc2N(c2cccc(-c3cccc(N(c4ccc5c(c4)sc4ccccc45)c4ccccc4-c4ccccc4)c3)c2)c2ccc3c(c2)sc2ccccc23)cc1] 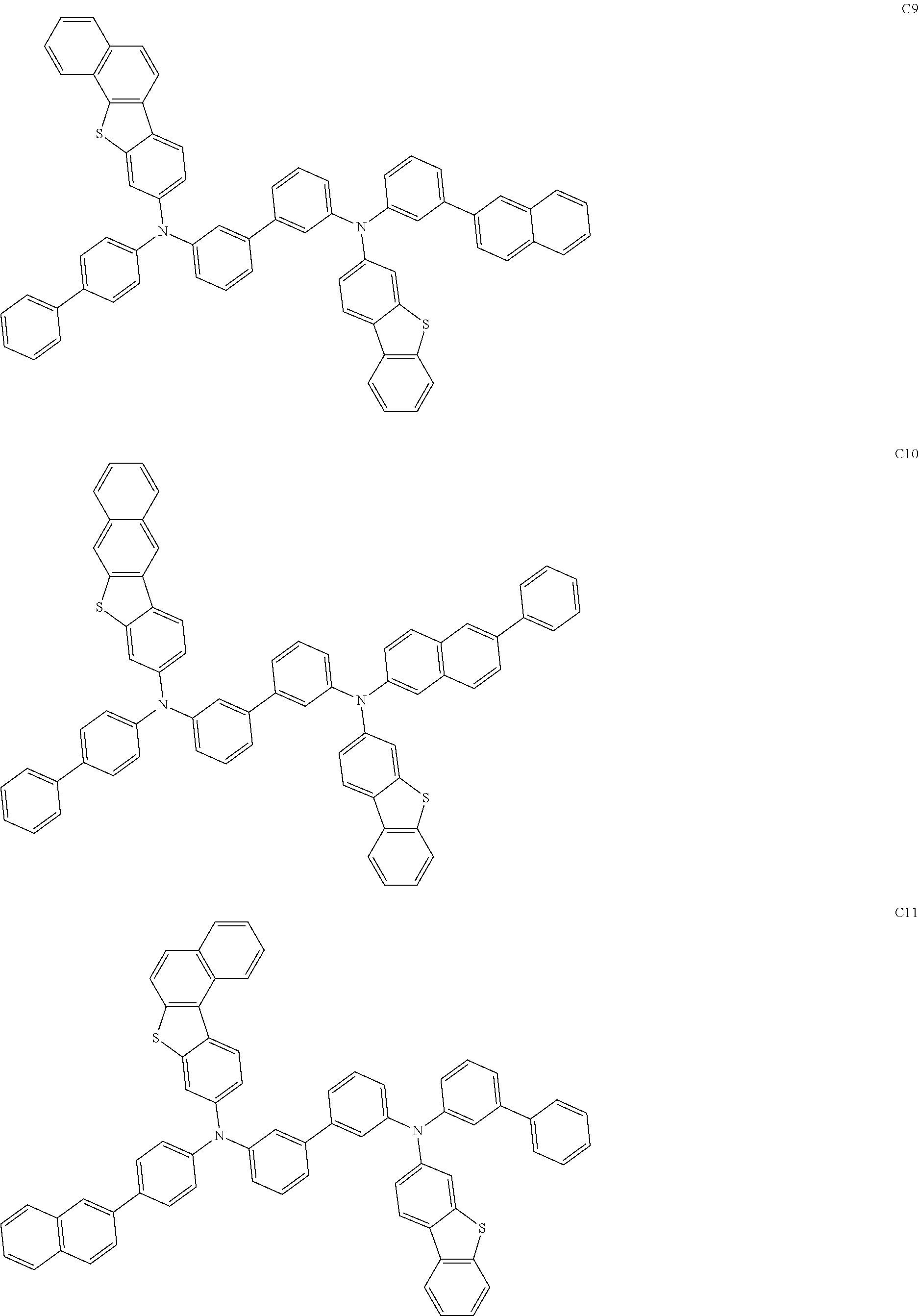 [SMILES: c1ccc(-c2ccc(N(c3cccc(-c4cccc(N(c5cccc(-c6ccc7ccccc7c6)c5)c5ccc6c(c5)sc5ccccc56)c4)c3)c3ccc4c(c3)sc3c5ccccc5ccc43)cc2)cc1 ; c1ccc(-c2ccc(N(c3cccc(-c4cccc(N(c5ccc6cc(-c7ccccc7)ccc6c5)c5ccc6c(c5)sc5ccccc56)c4)c3)c3ccc4c(c3)sc3cc5ccccc5cc34)cc2)cc1 ; c1ccc(-c2cccc(N(c3cccc(-c4cccc(N(c5ccc(-c6ccc7ccccc7c6)cc5)c5ccc6c(c5)sc5ccc7ccccc7c56)c4)c3)c3ccc4c(c3)sc3ccccc34)c2)cc1] 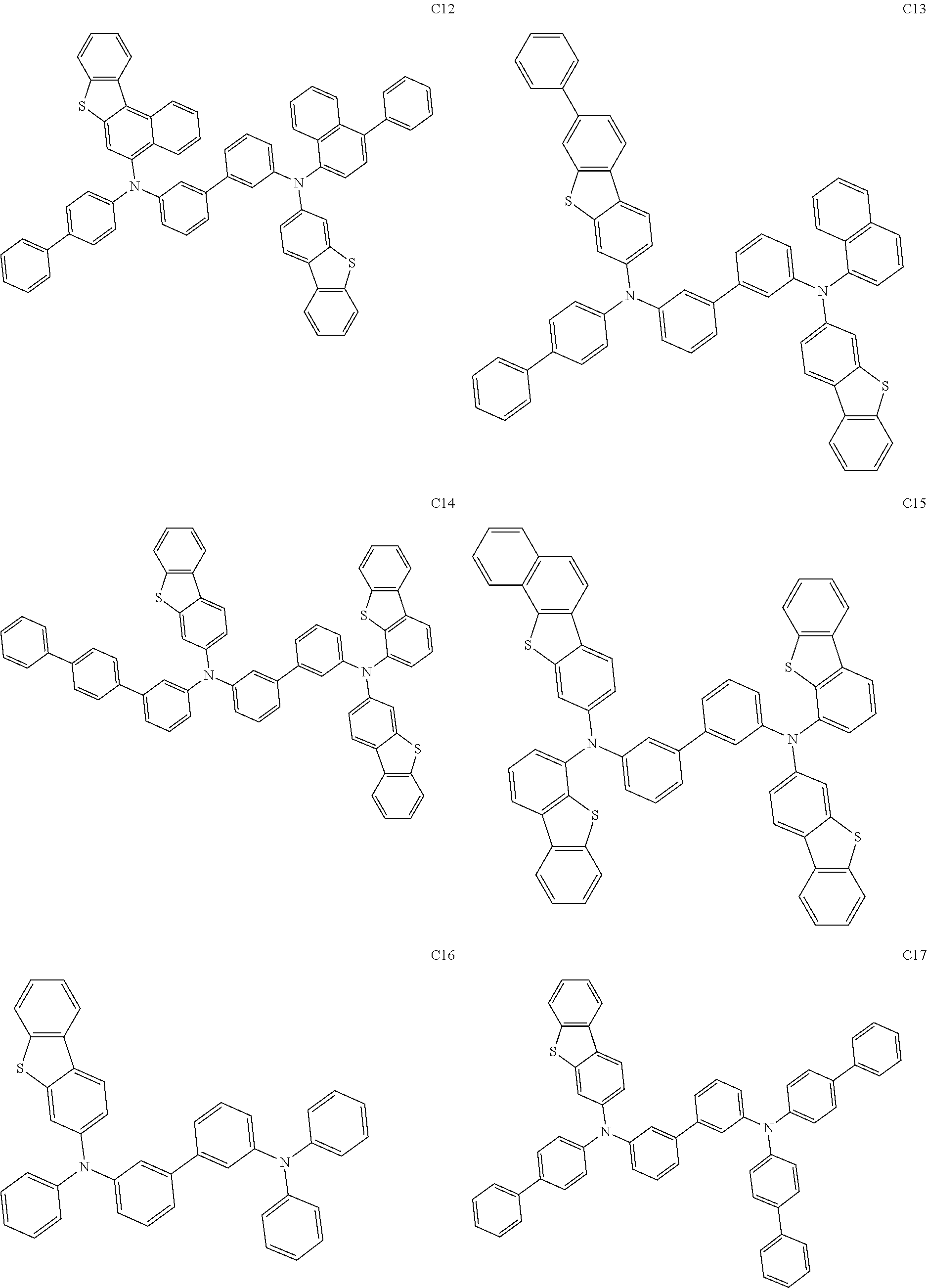 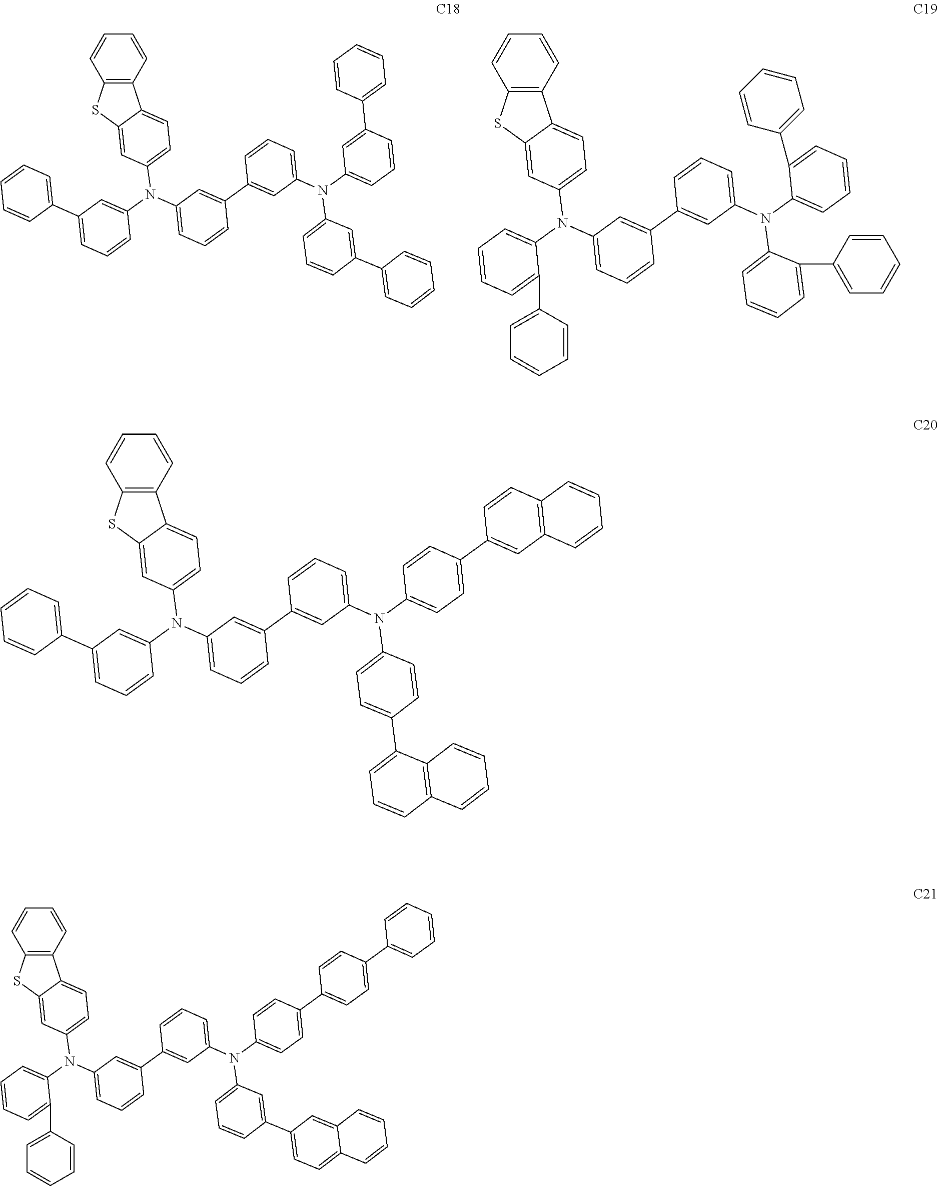 [SMILES: c1ccc(-c2cccc(N(c3cccc(-c4ccccc4)c3)c3cccc(-c4cccc(N(c5cccc(-c6ccccc6)c5)c5ccc6c(c5)sc5ccccc56)c4)c3)c2)cc1 ; c1ccc(-c2ccccc2N(c2cccc(-c3cccc(N(c4ccccc4-c4ccccc4)c4ccccc4-c4ccccc4)c3)c2)c2ccc3c(c2)sc2ccccc23)cc1 ; c1ccc(-c2cccc(N(c3cccc(-c4cccc(N(c5ccc(-c6ccc7ccccc7c6)cc5)c5ccc(-c6cccc7ccccc67)cc5)c4)c3)c3ccc4c(c3)sc3ccccc34)c2)cc1 ; c1ccc(-c2ccc(-c3ccc(N(c4cccc(-c5cccc(N(c6ccc7c(c6)sc6ccccc67)c6ccccc6-c6ccccc6)c5)c4)c4cccc(-c5ccc6ccccc6c5)c4)cc3)cc2)cc1] 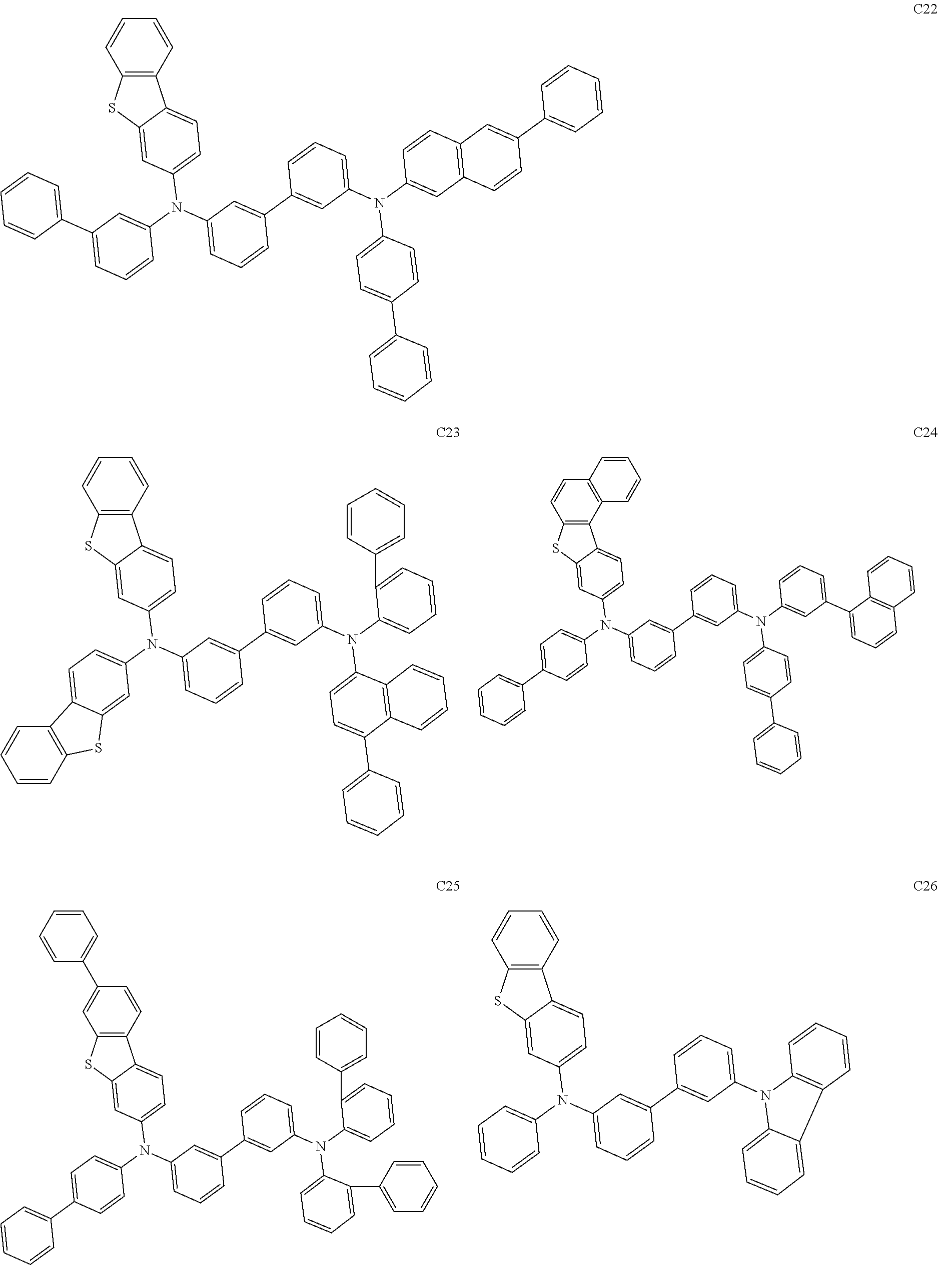 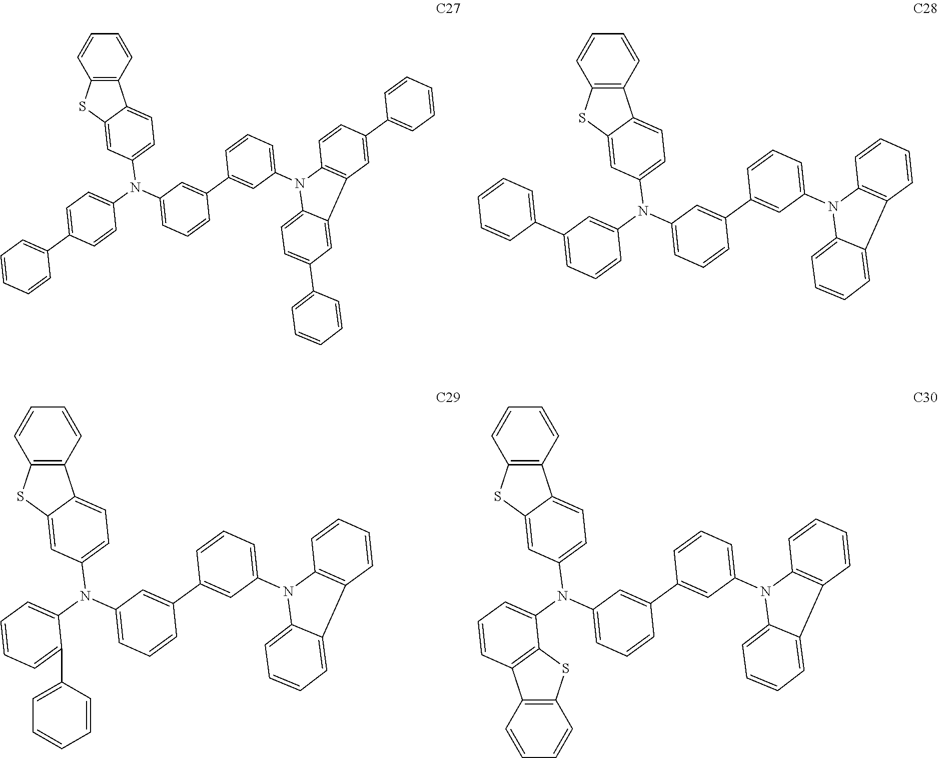 [SMILES: c1ccc(-c2ccc(N(c3cccc(-c4cccc(-n5c6ccc(-c7ccccc7)cc6c6cc(-c7ccccc7)ccc65)c4)c3)c3ccc4c(c3)sc3ccccc34)cc2)cc1 ; c1ccc(-c2cccc(N(c3cccc(-c4cccc(-n5c6ccccc6c6ccccc65)c4)c3)c3ccc4c(c3)sc3ccccc34)c2)cc1 ; c1ccc(-c2ccccc2N(c2cccc(-c3cccc(-n4c5ccccc5c5ccccc54)c3)c2)c2ccc3c(c2)sc2ccccc23)cc1 ; c1cc(-c2cccc(-n3c4ccccc4c4ccccc43)c2)cc(N(c2ccc3c(c2)sc2ccccc23)c2cccc3c2sc2ccccc23)c1] 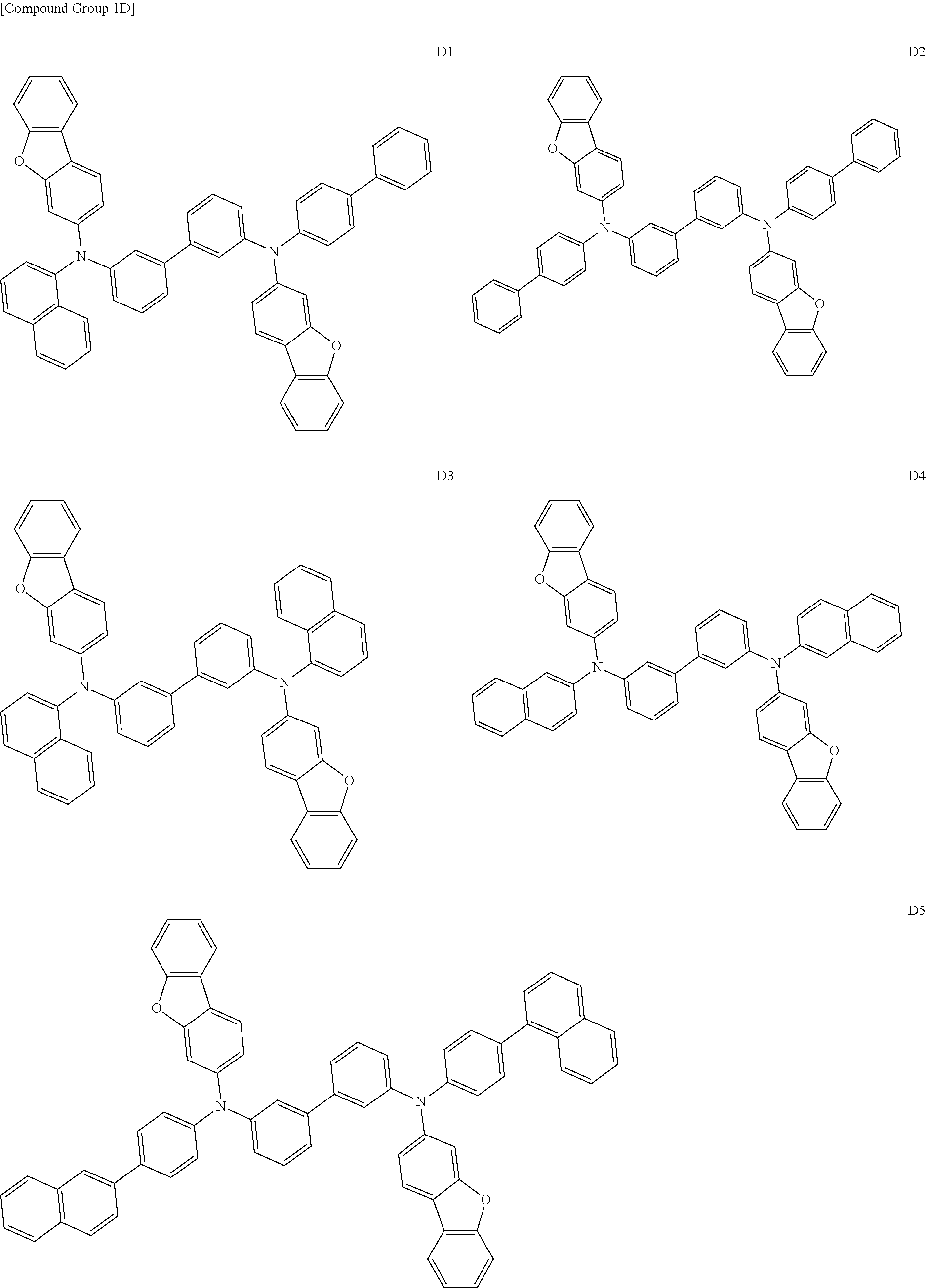 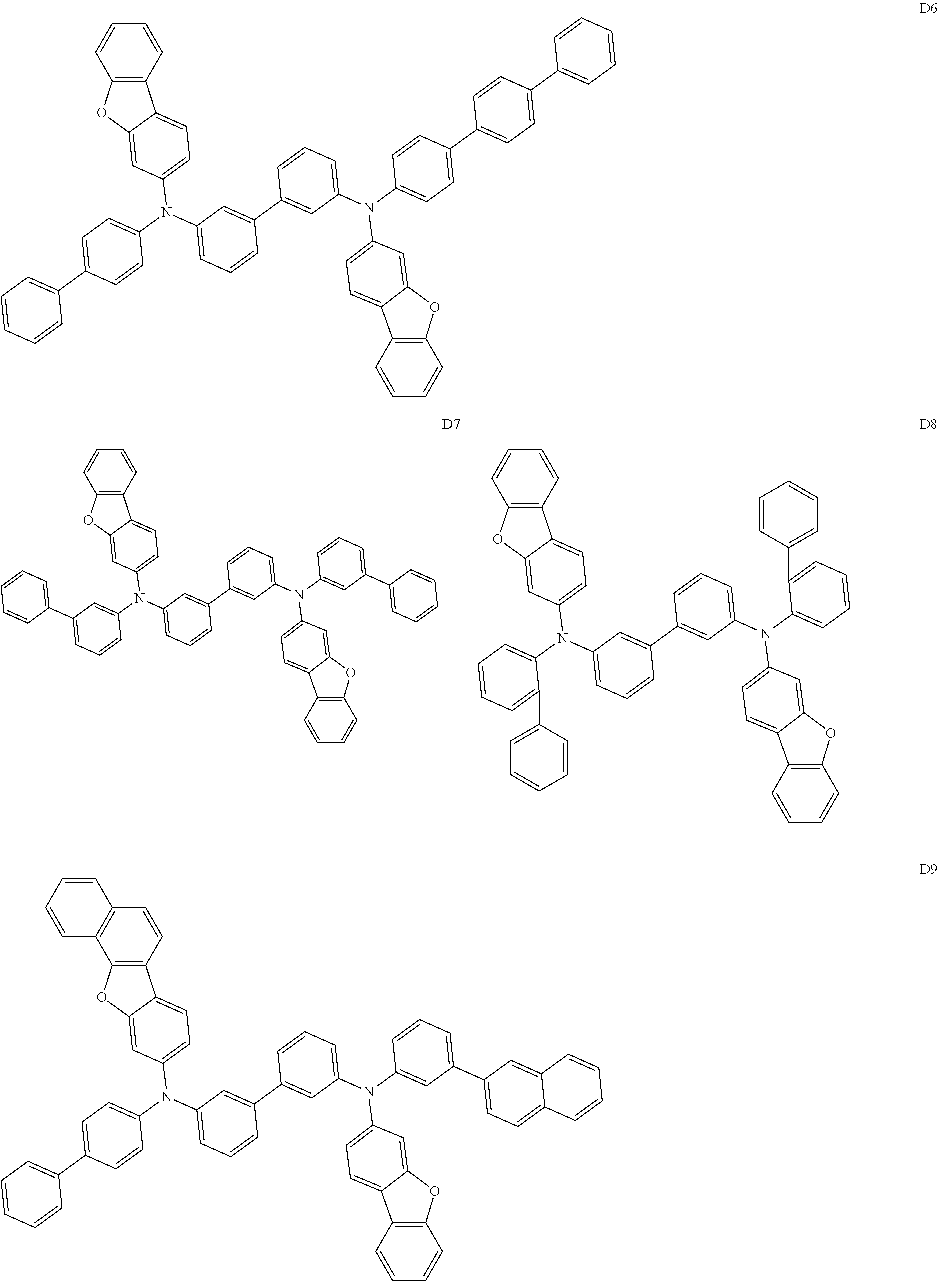 [SMILES: c1ccc(-c2ccc(-c3ccc(N(c4cccc(-c5cccc(N(c6ccc(-c7ccccc7)cc6)c6ccc7c(c6)oc6ccccc67)c5)c4)c4ccc5c(c4)oc4ccccc45)cc3)cc2)cc1 ; c1ccc(-c2cccc(N(c3cccc(-c4cccc(N(c5cccc(-c6ccccc6)c5)c5ccc6c(c5)oc5ccccc56)c4)c3)c3ccc4c(c3)oc3ccccc34)c2)cc1 ; c1ccc(-c2ccccc2N(c2cccc(-c3cccc(N(c4ccc5c(c4)oc4ccccc45)c4ccccc4-c4ccccc4)c3)c2)c2ccc3c(c2)oc2ccccc23)cc1 ; c1ccc(-c2ccc(N(c3cccc(-c4cccc(N(c5cccc(-c6ccc7ccccc7c6)c5)c5ccc6c(c5)oc5ccccc56)c4)c3)c3ccc4c(c3)oc3c5ccccc5ccc43)cc2)cc1] 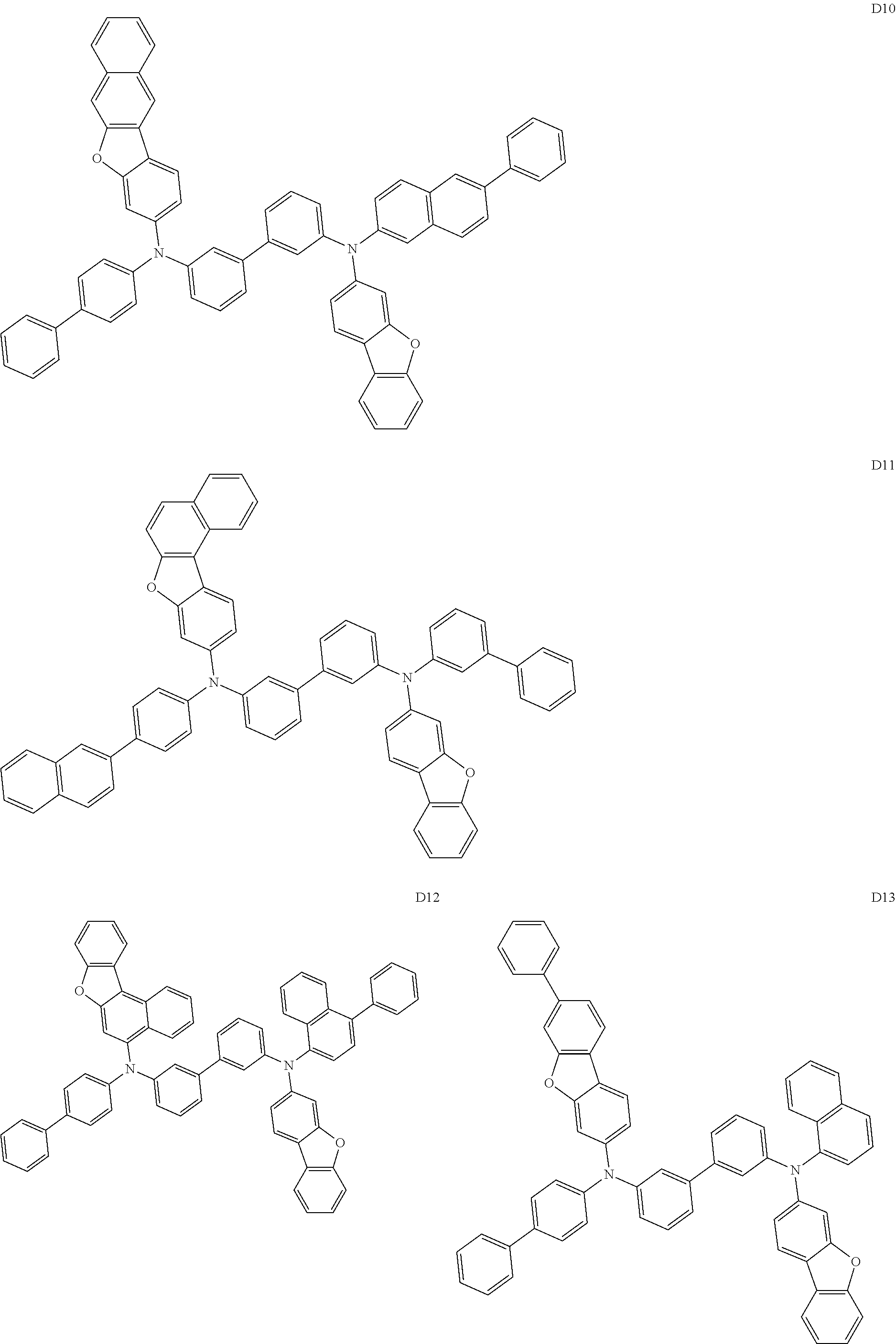 [SMILES: c1ccc(-c2ccc(N(c3cccc(-c4cccc(N(c5ccc6cc(-c7ccccc7)ccc6c5)c5ccc6c(c5)oc5ccccc56)c4)c3)c3ccc4c(c3)oc3cc5ccccc5cc34)cc2)cc1 ; c1ccc(-c2cccc(N(c3cccc(-c4cccc(N(c5ccc(-c6ccc7ccccc7c6)cc5)c5ccc6c(c5)oc5ccc7ccccc7c56)c4)c3)c3ccc4c(c3)oc3ccccc34)c2)cc1 ; c1ccc(-c2ccc(N(c3cccc(-c4cccc(N(c5ccc6c(c5)oc5ccccc56)c5ccc(-c6ccccc6)c6ccccc56)c4)c3)c3cc4oc5ccccc5c4c4ccccc34)cc2)cc1 ; c1ccc(-c2ccc(N(c3cccc(-c4cccc(N(c5ccc6c(c5)oc5ccccc56)c5cccc6ccccc56)c4)c3)c3ccc4c(c3)oc3cc(-c5ccccc5)ccc34)cc2)cc1] 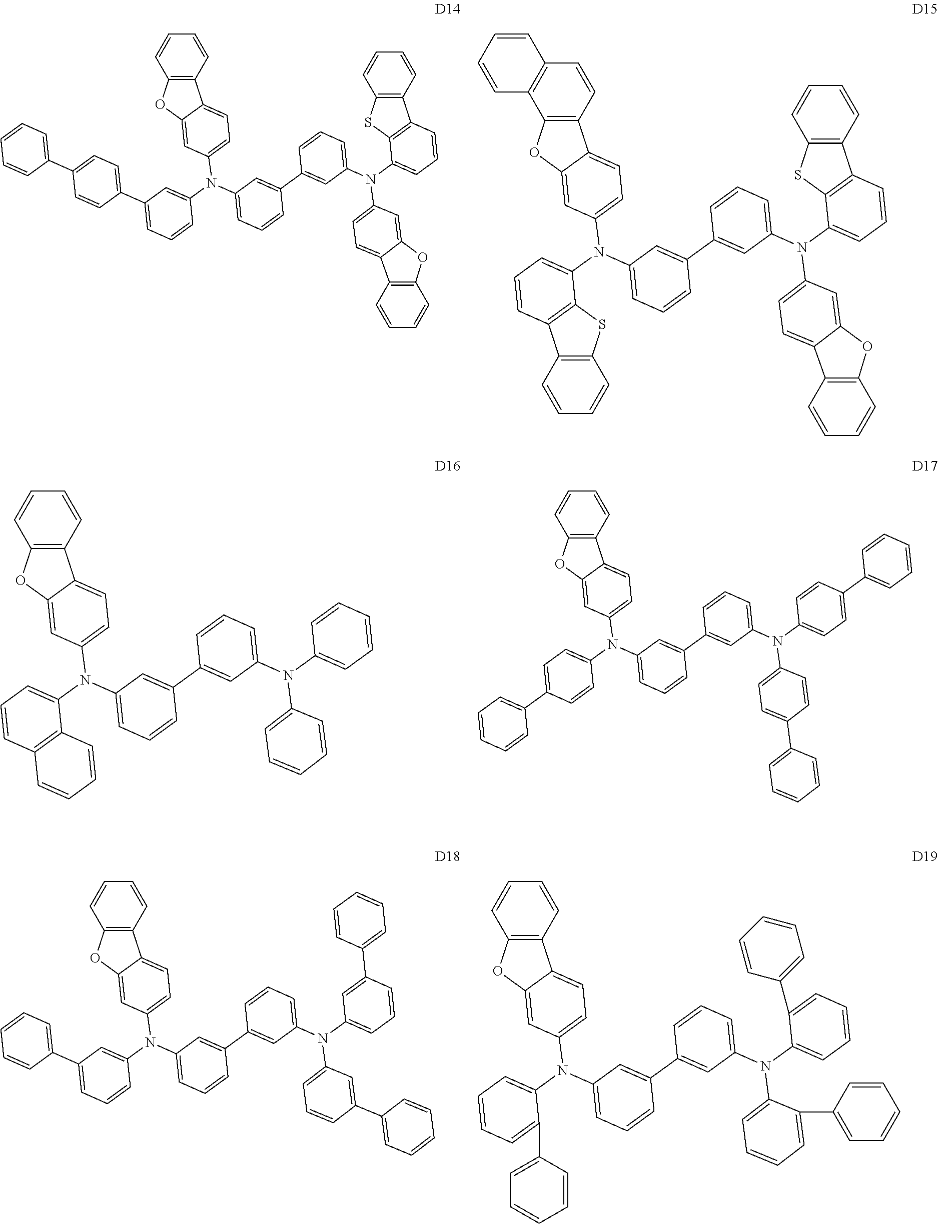 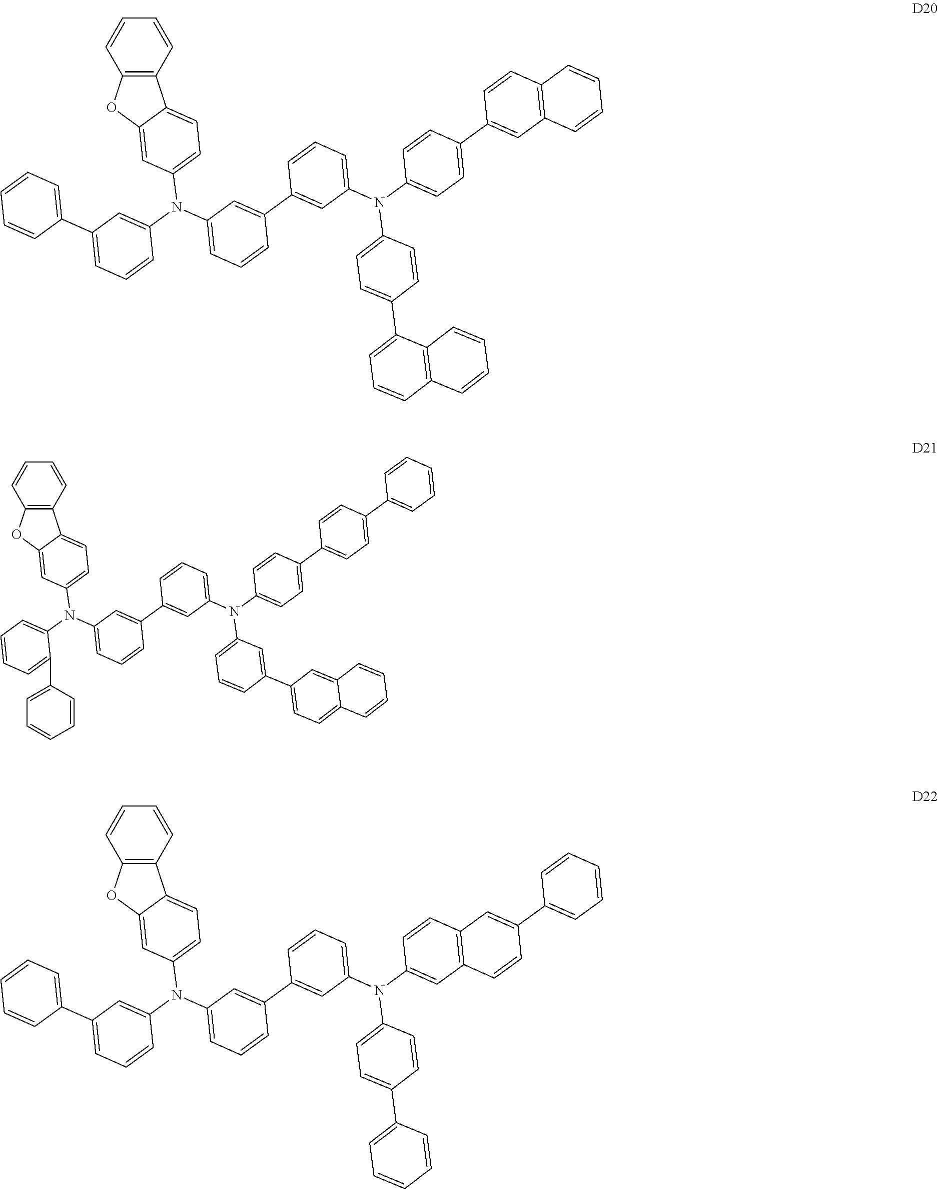 [SMILES: c1ccc(-c2cccc(N(c3cccc(-c4cccc(N(c5ccc(-c6ccc7ccccc7c6)cc5)c5ccc(-c6cccc7ccccc67)cc5)c4)c3)c3ccc4c(c3)oc3ccccc34)c2)cc1 ; c1ccc(-c2ccc(-c3ccc(N(c4cccc(-c5cccc(N(c6ccc7c(c6)oc6ccccc67)c6ccccc6-c6ccccc6)c5)c4)c4cccc(-c5ccc6ccccc6c5)c4)cc3)cc2)cc1 ; c1ccc(-c2ccc(N(c3cccc(-c4cccc(N(c5cccc(-c6ccccc6)c5)c5ccc6c(c5)oc5ccccc56)c4)c3)c3ccc4cc(-c5ccccc5)ccc4c3)cc2)cc1] 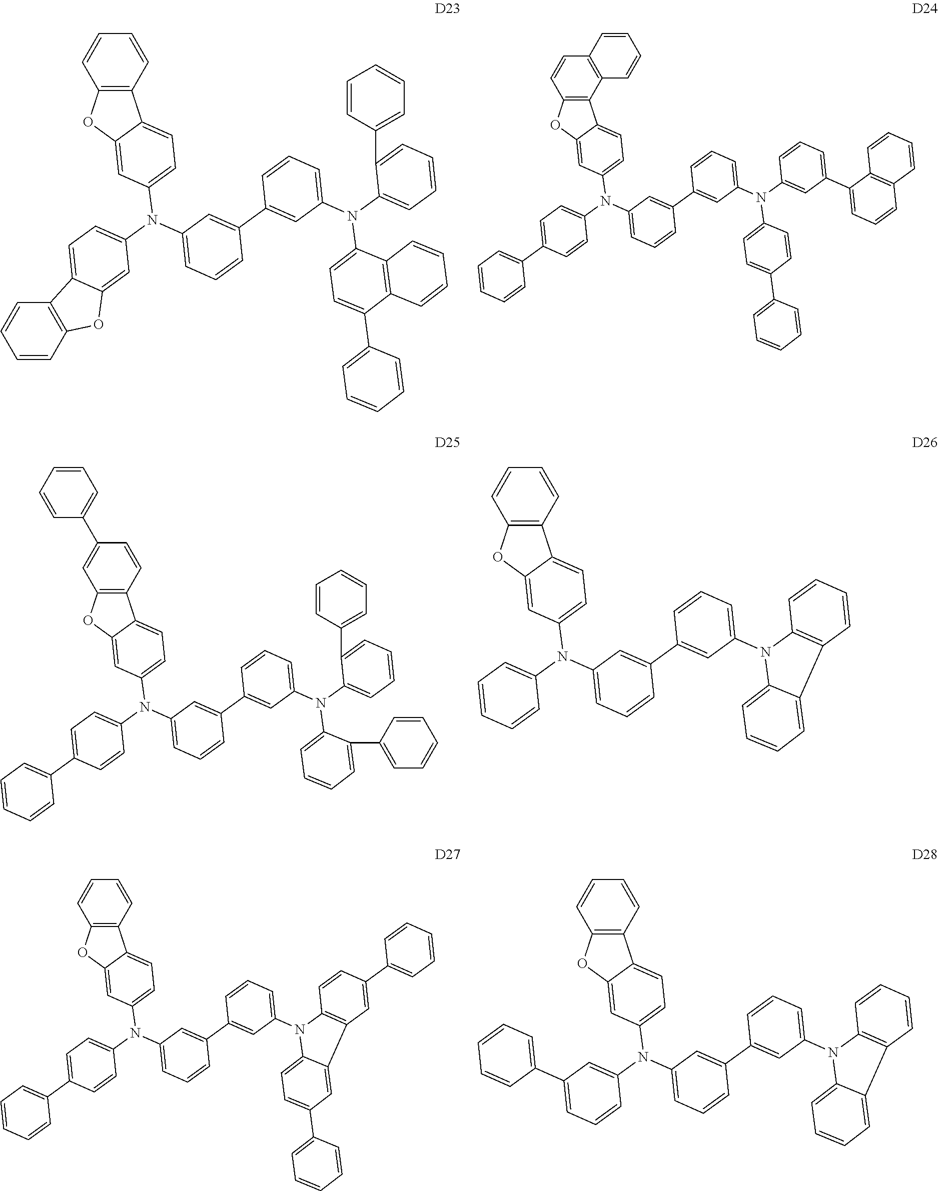 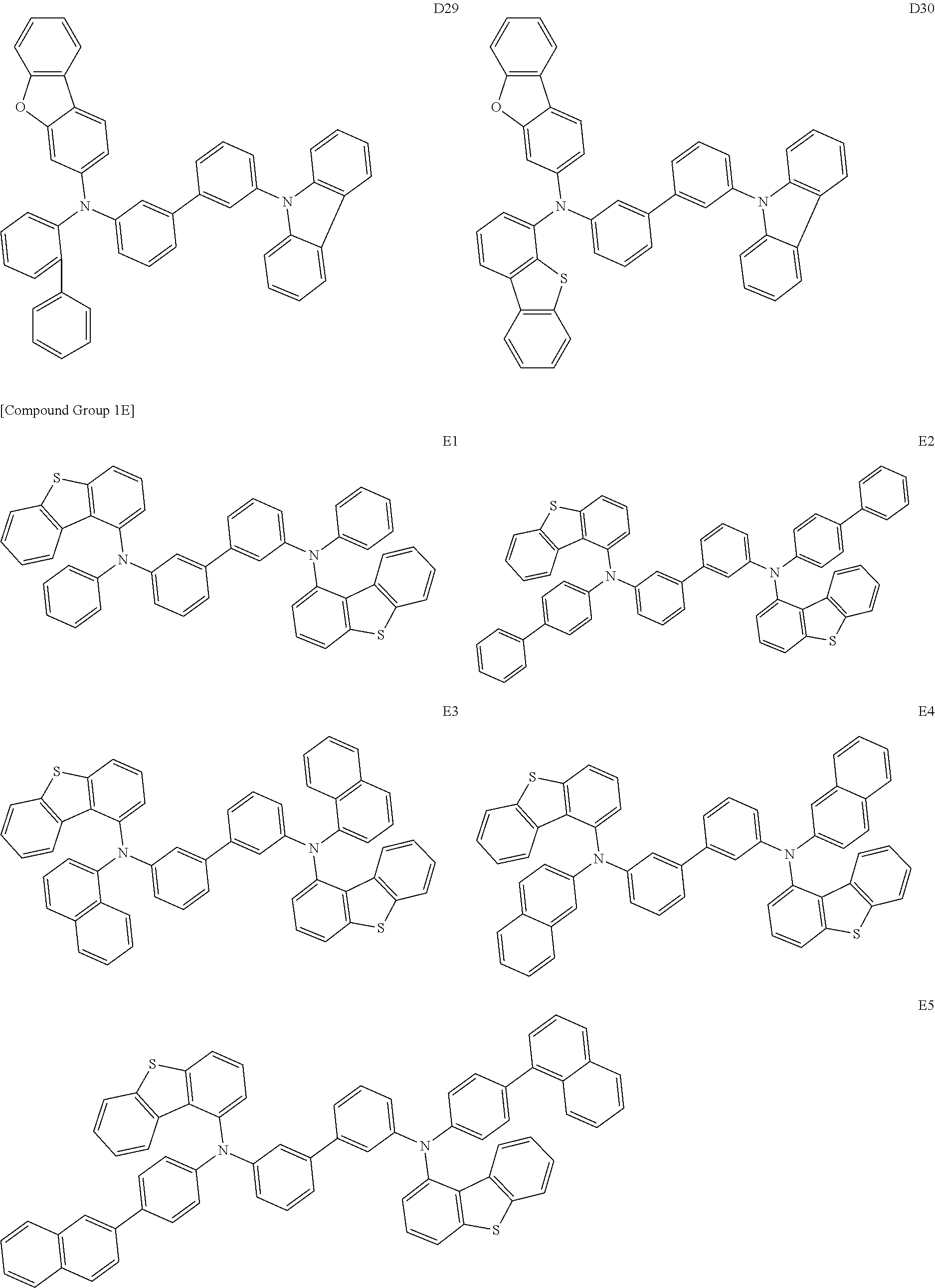 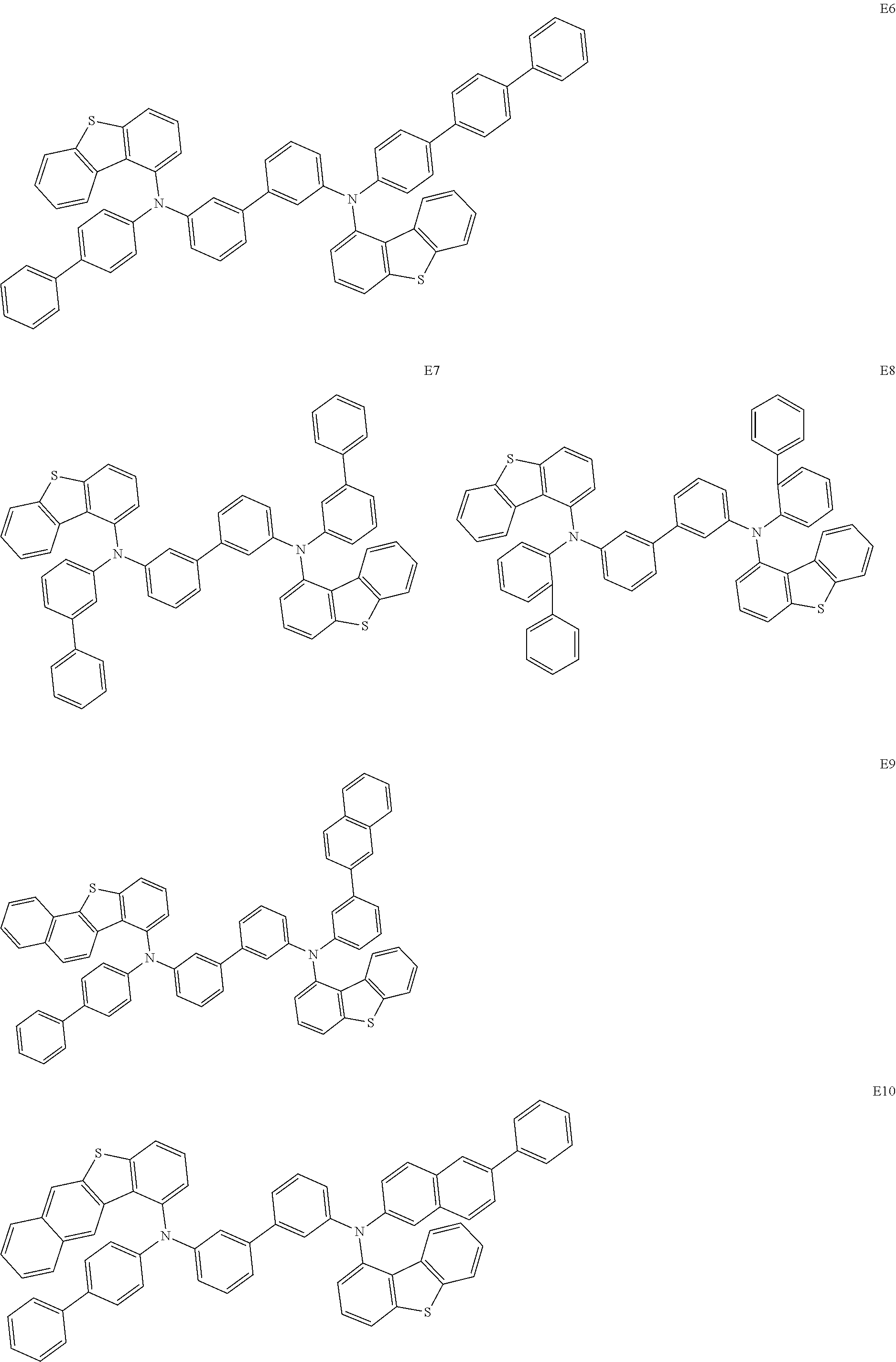 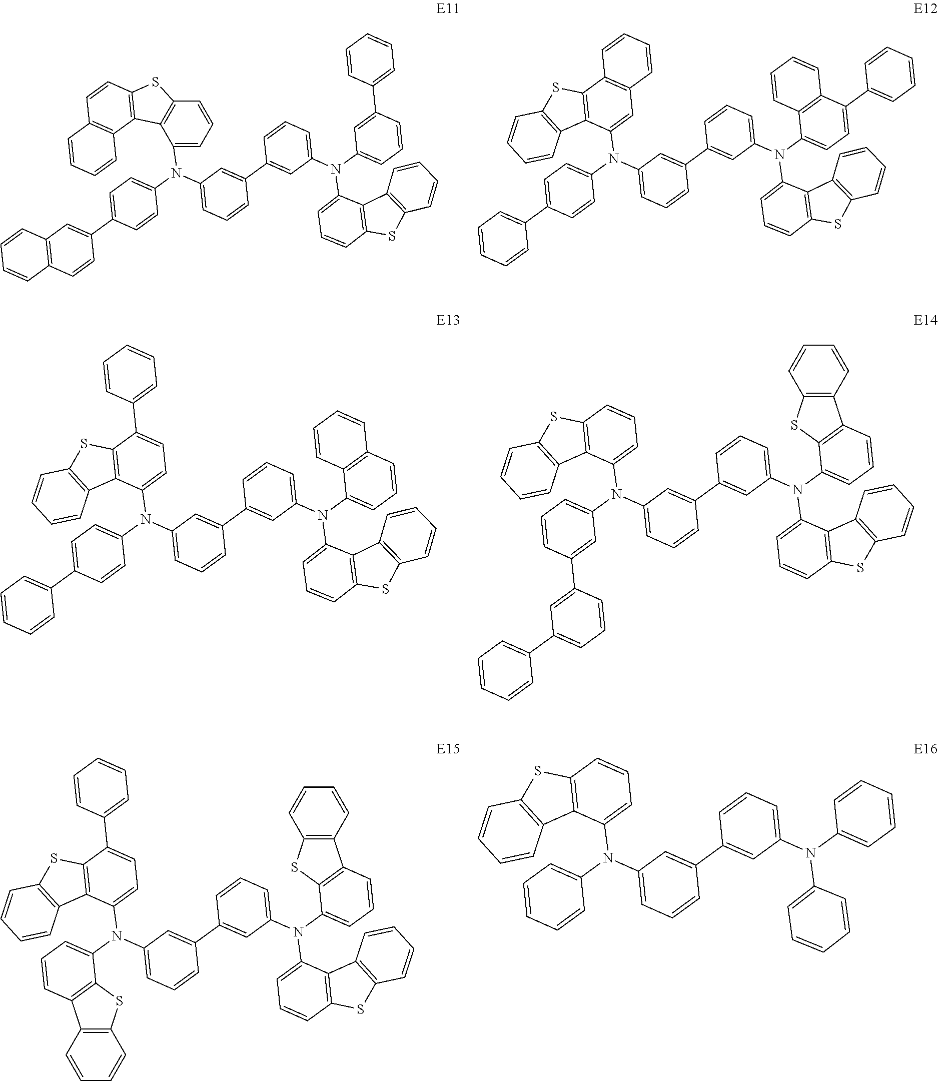 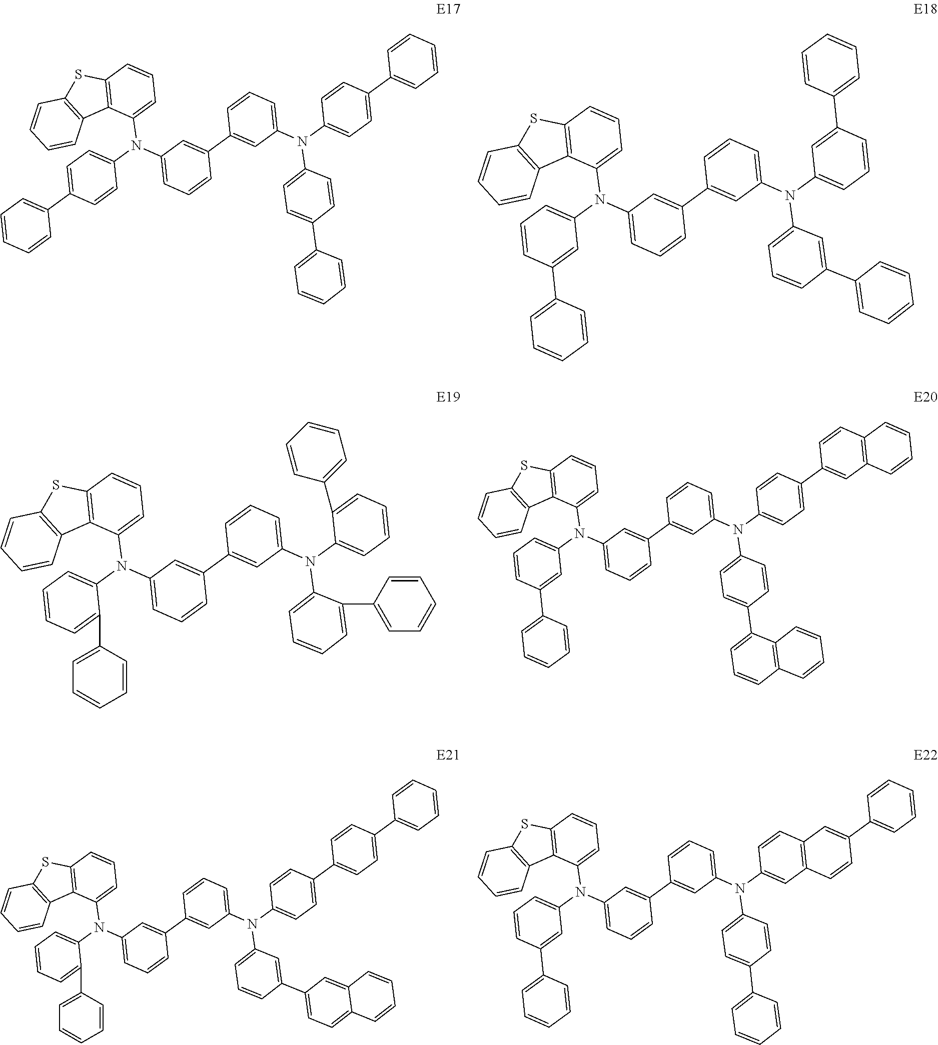 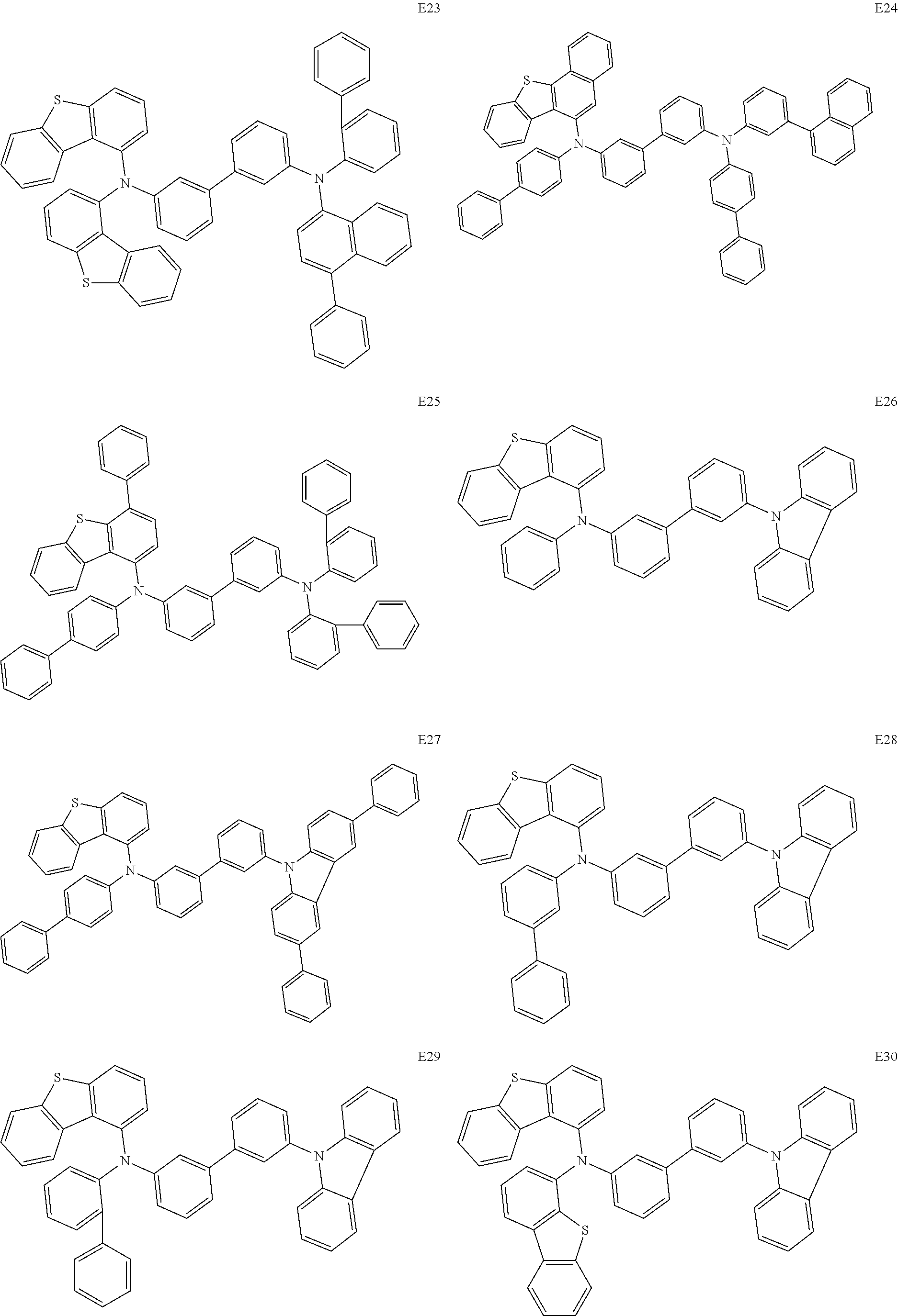 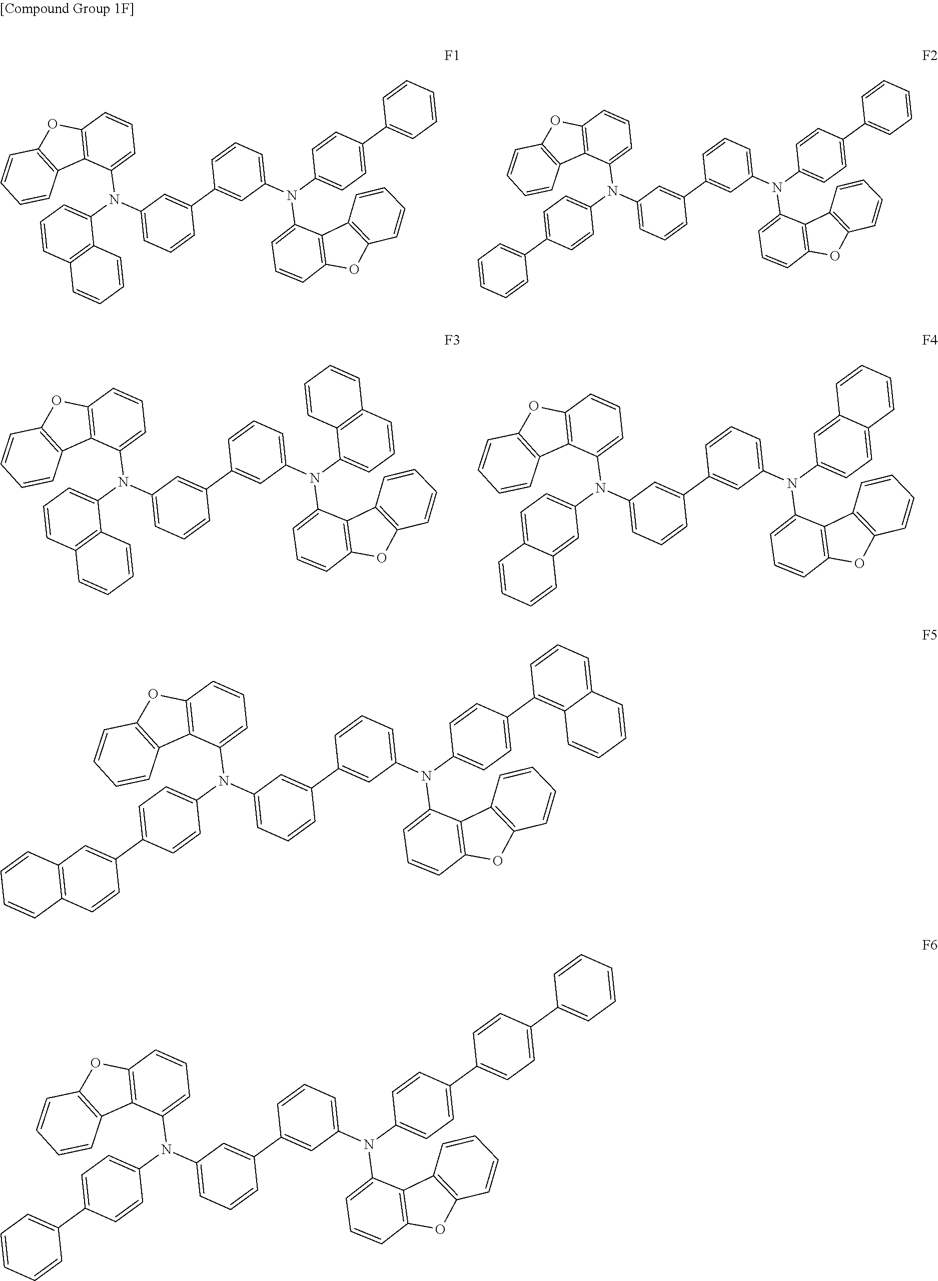 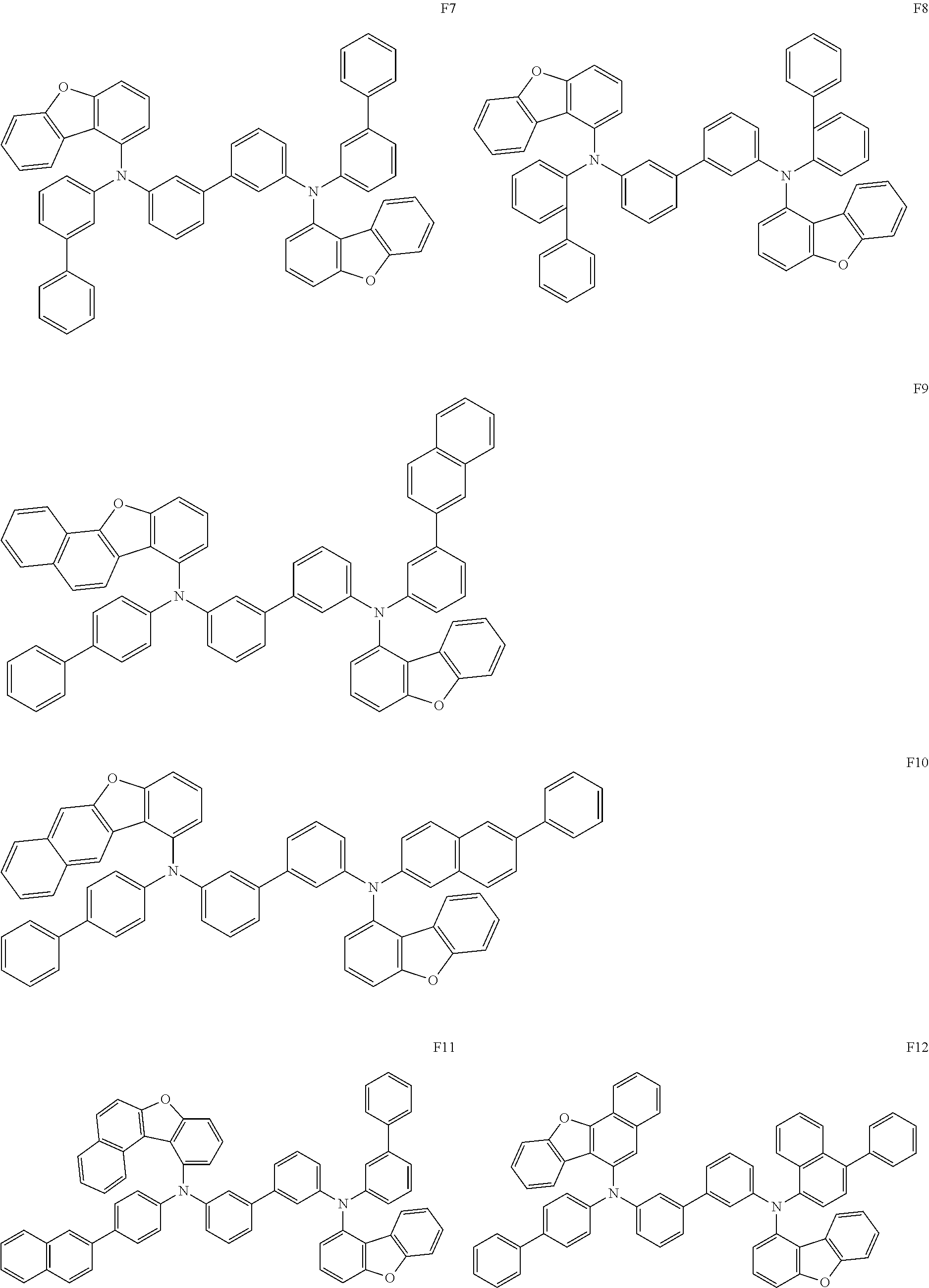 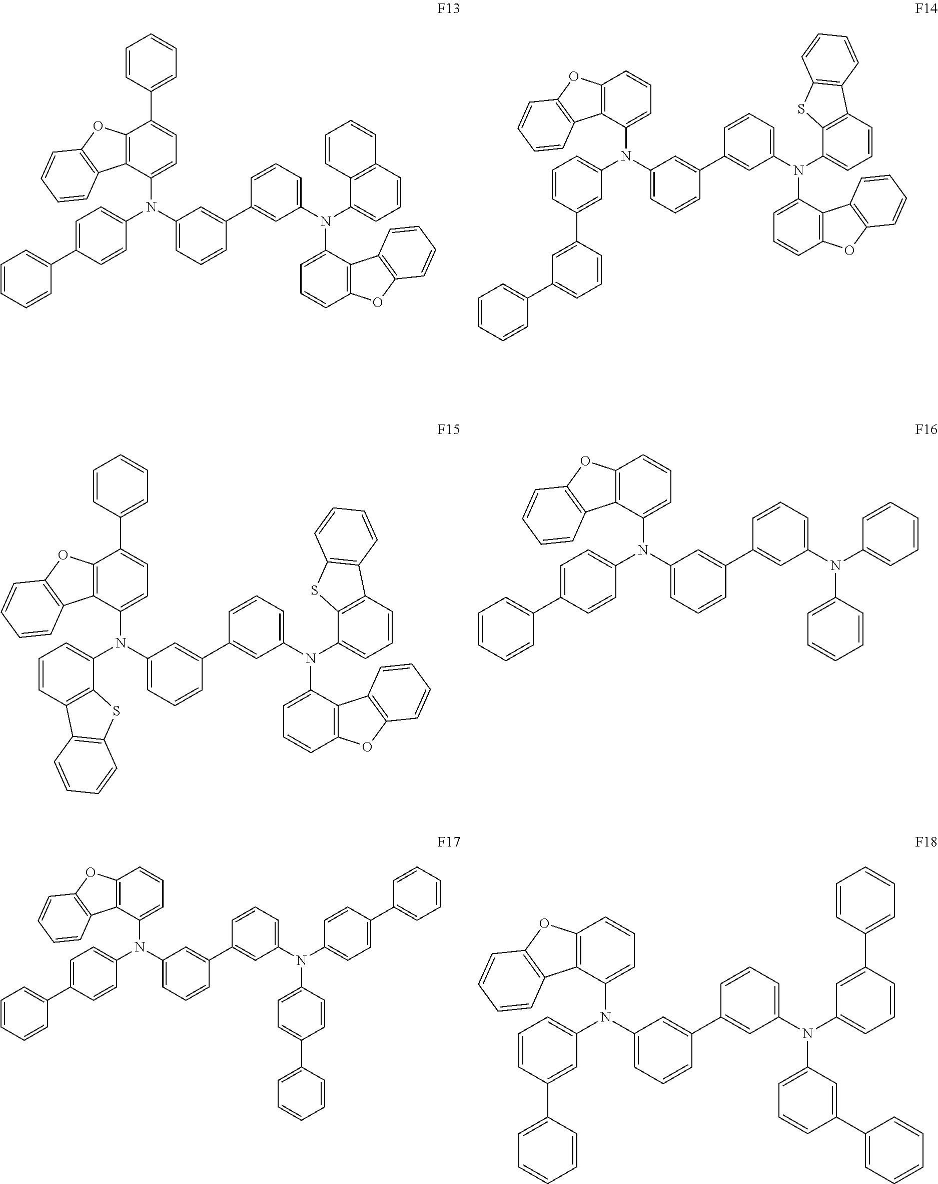 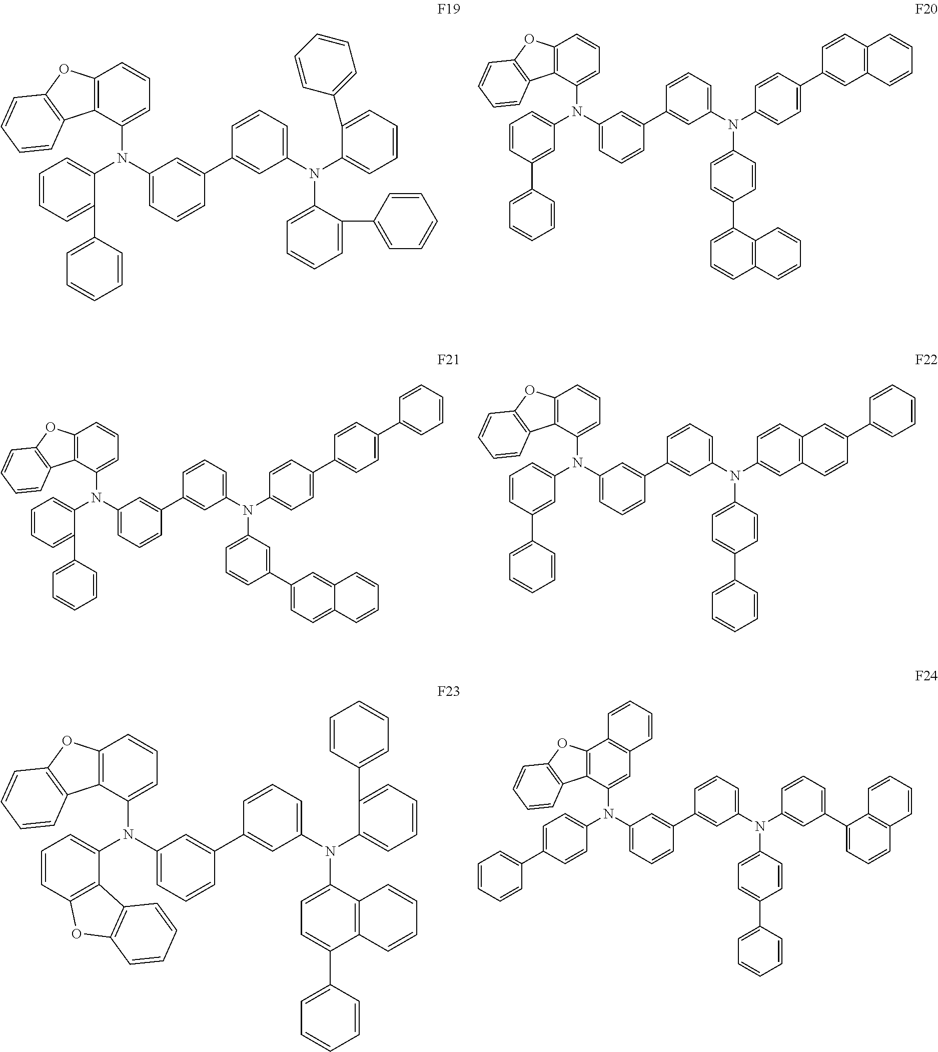 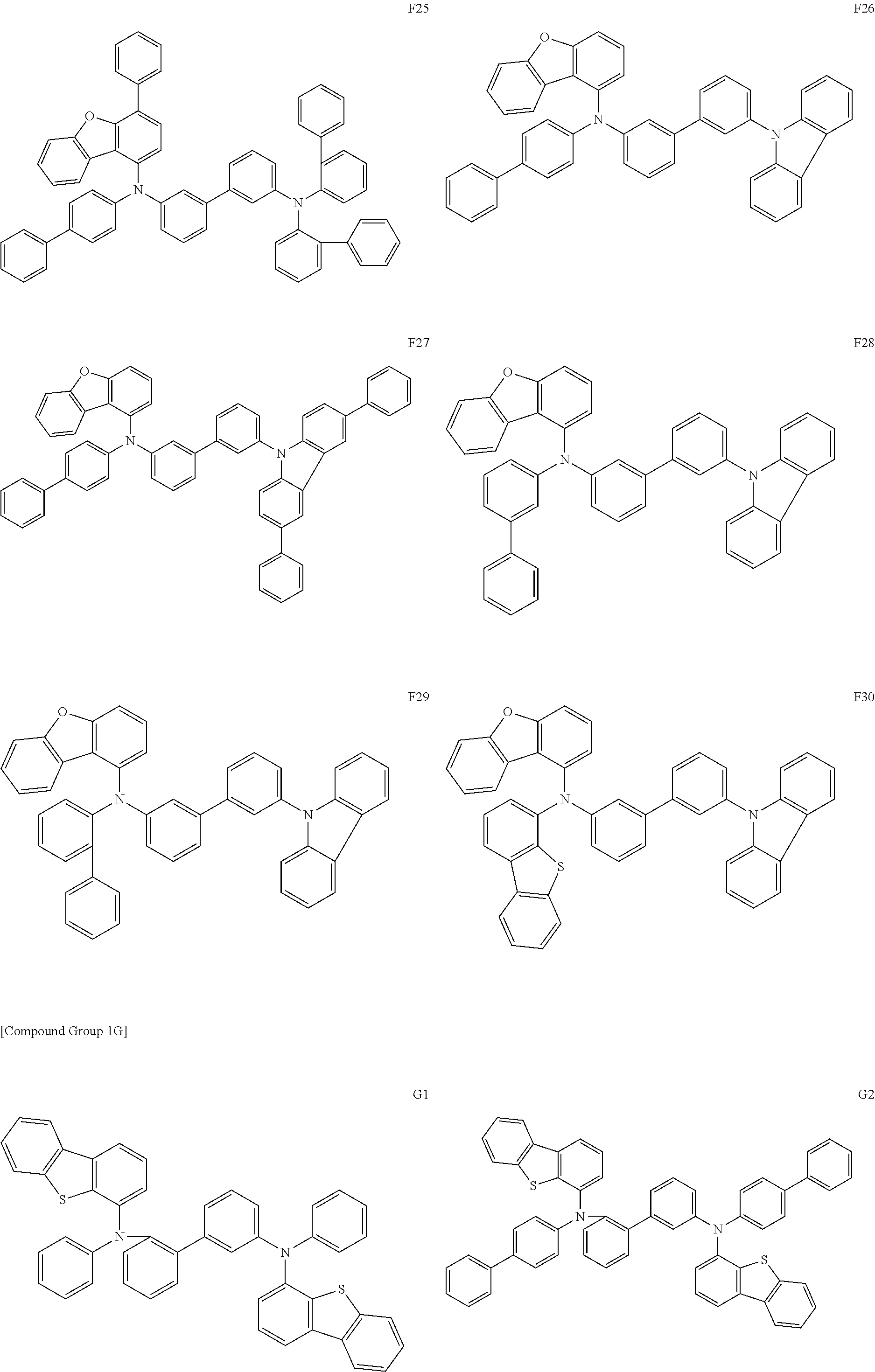 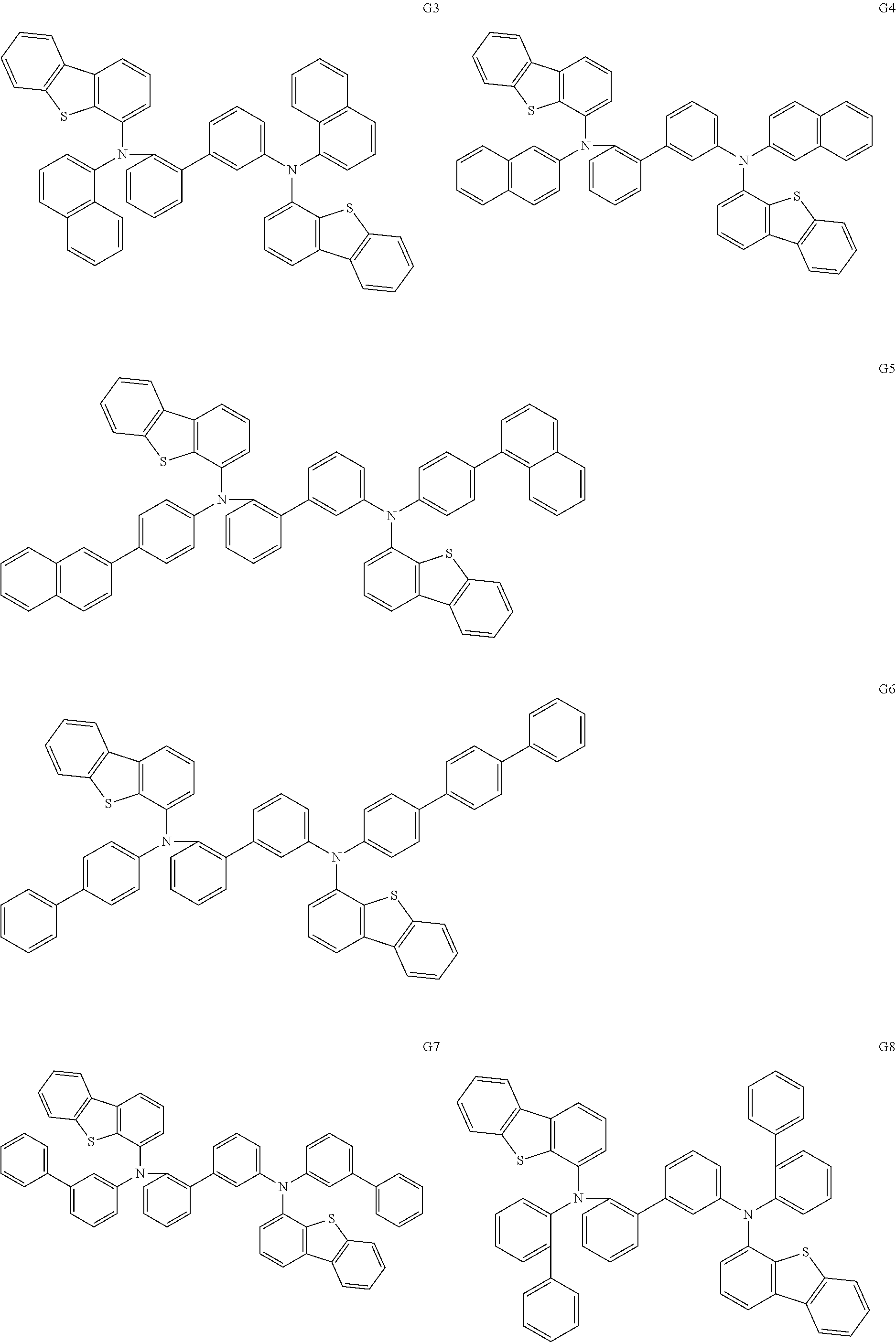 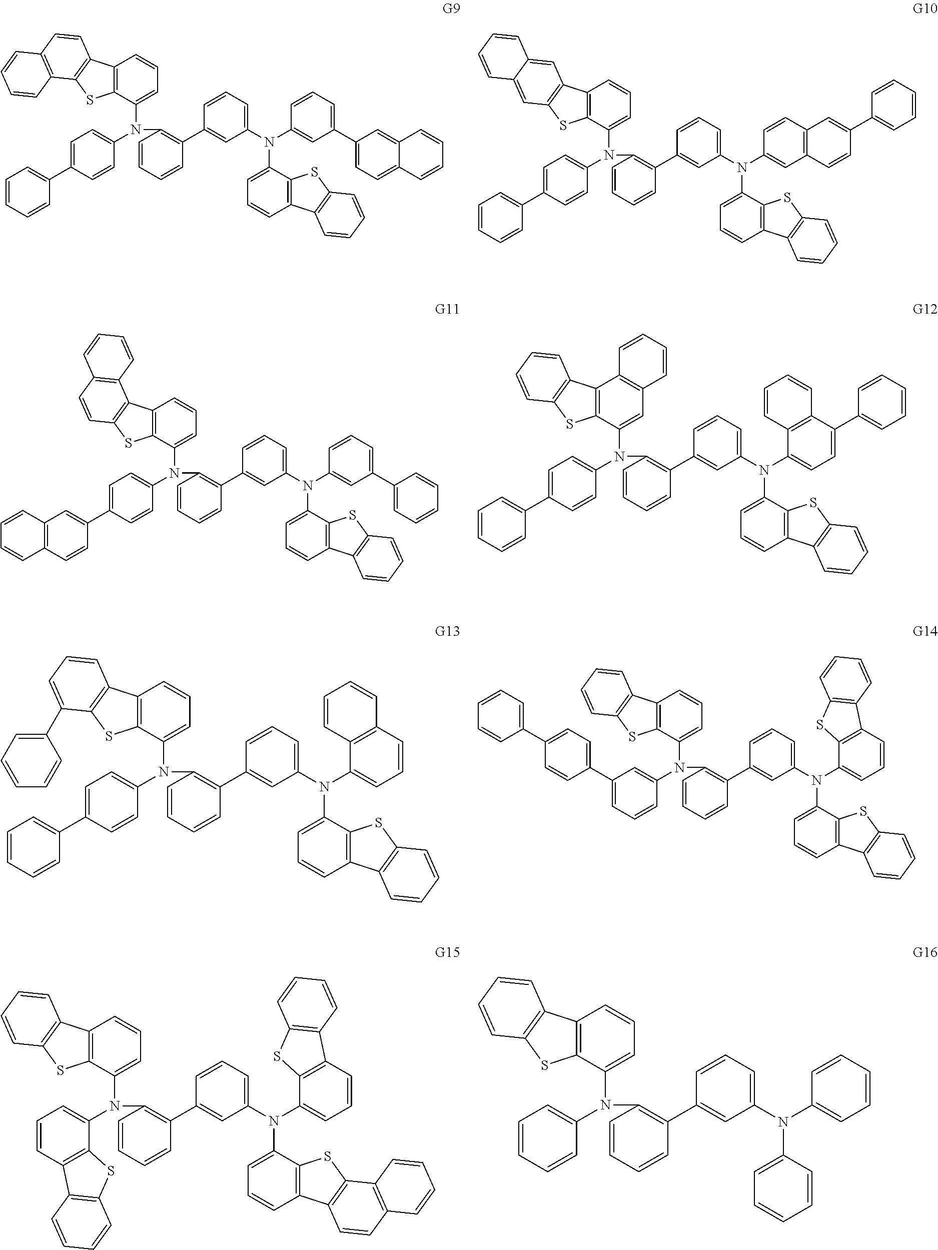 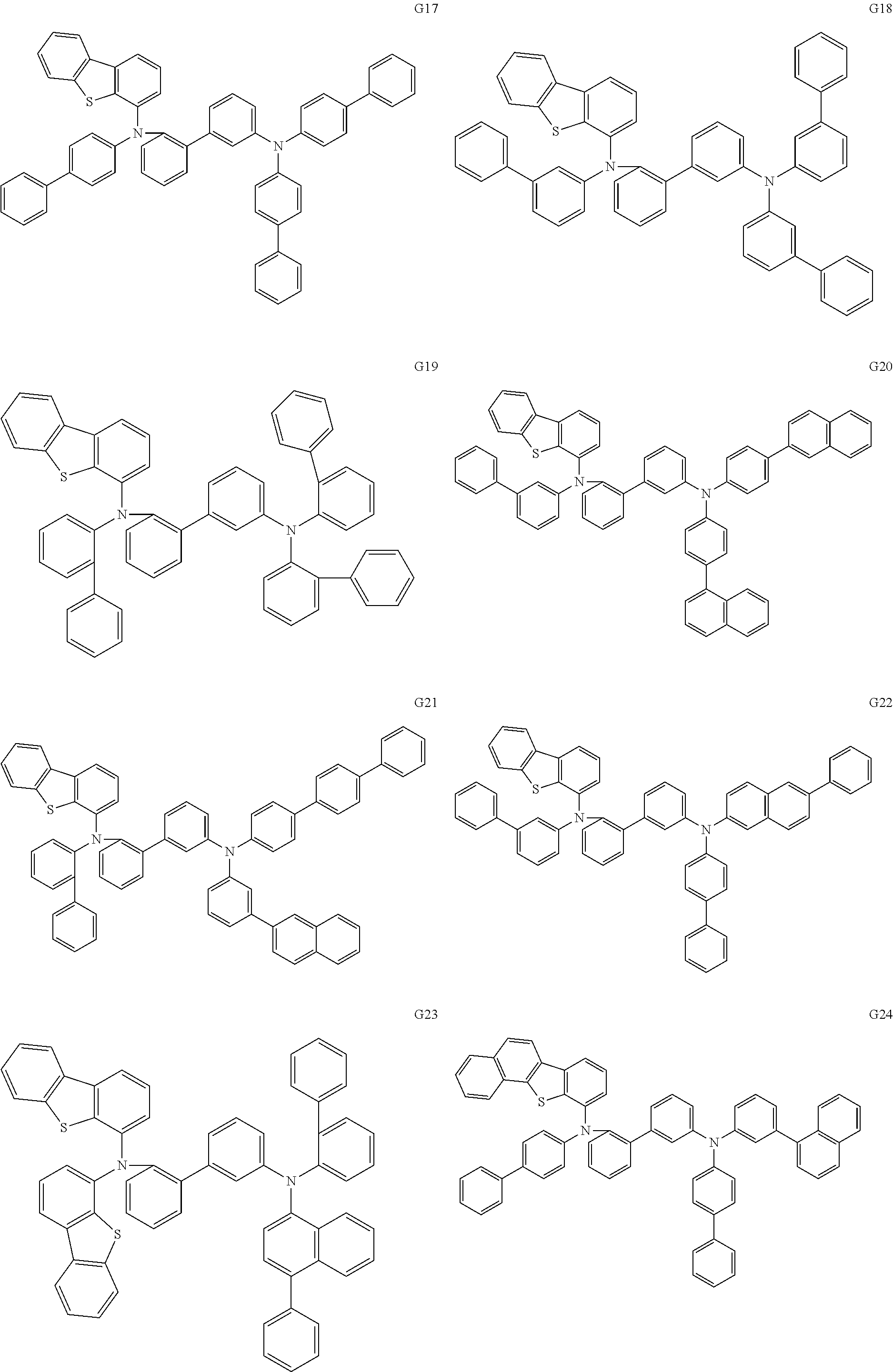 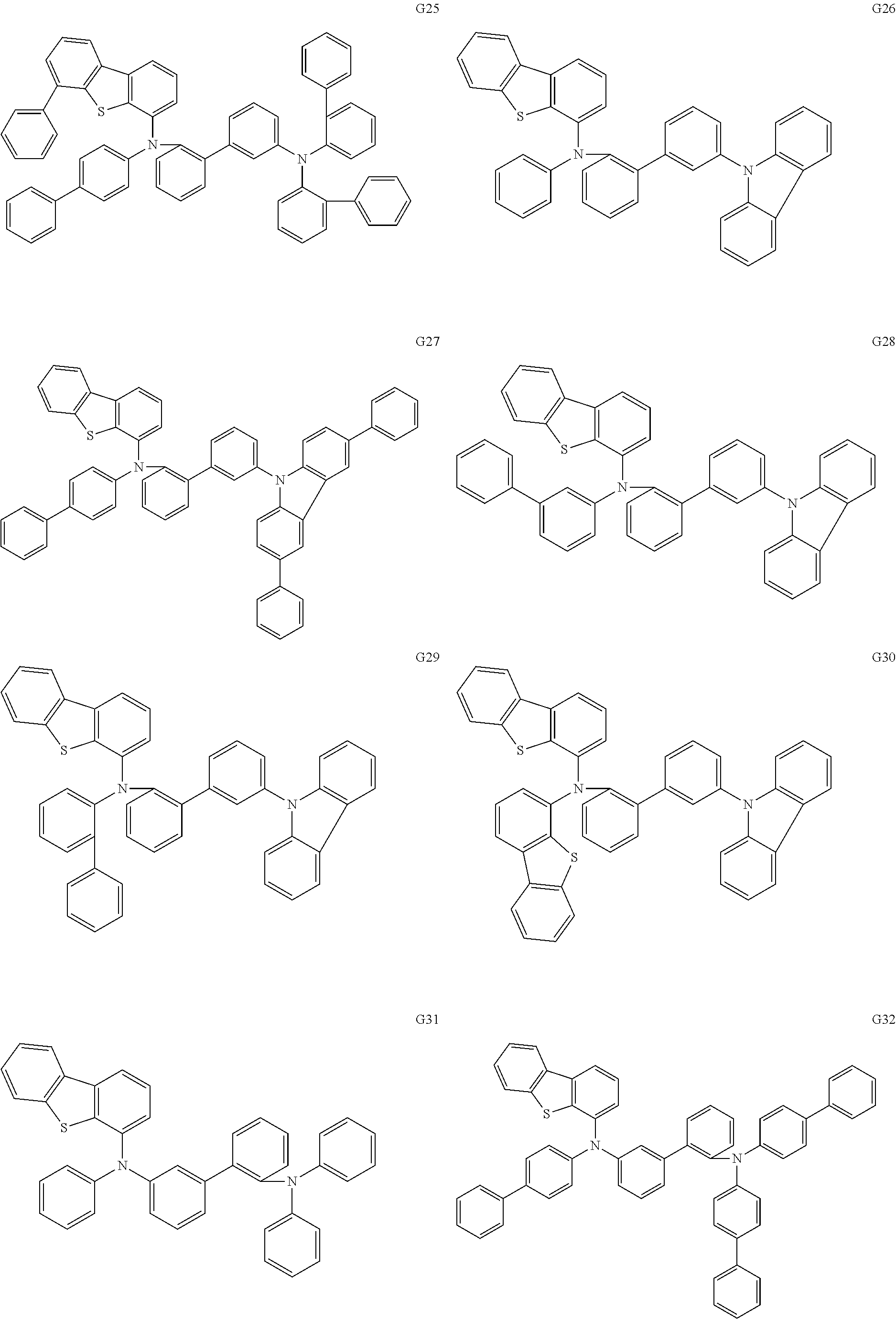 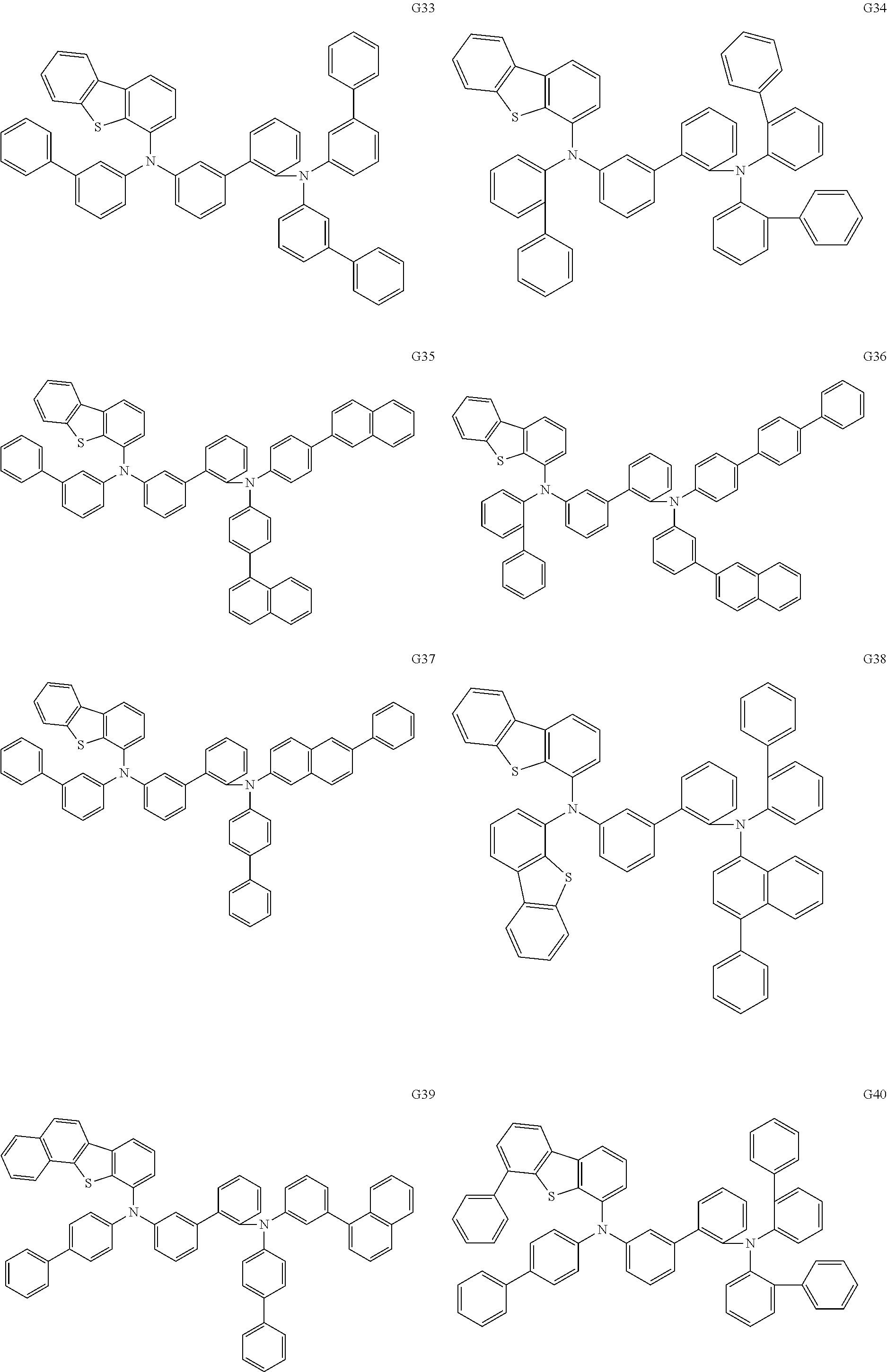 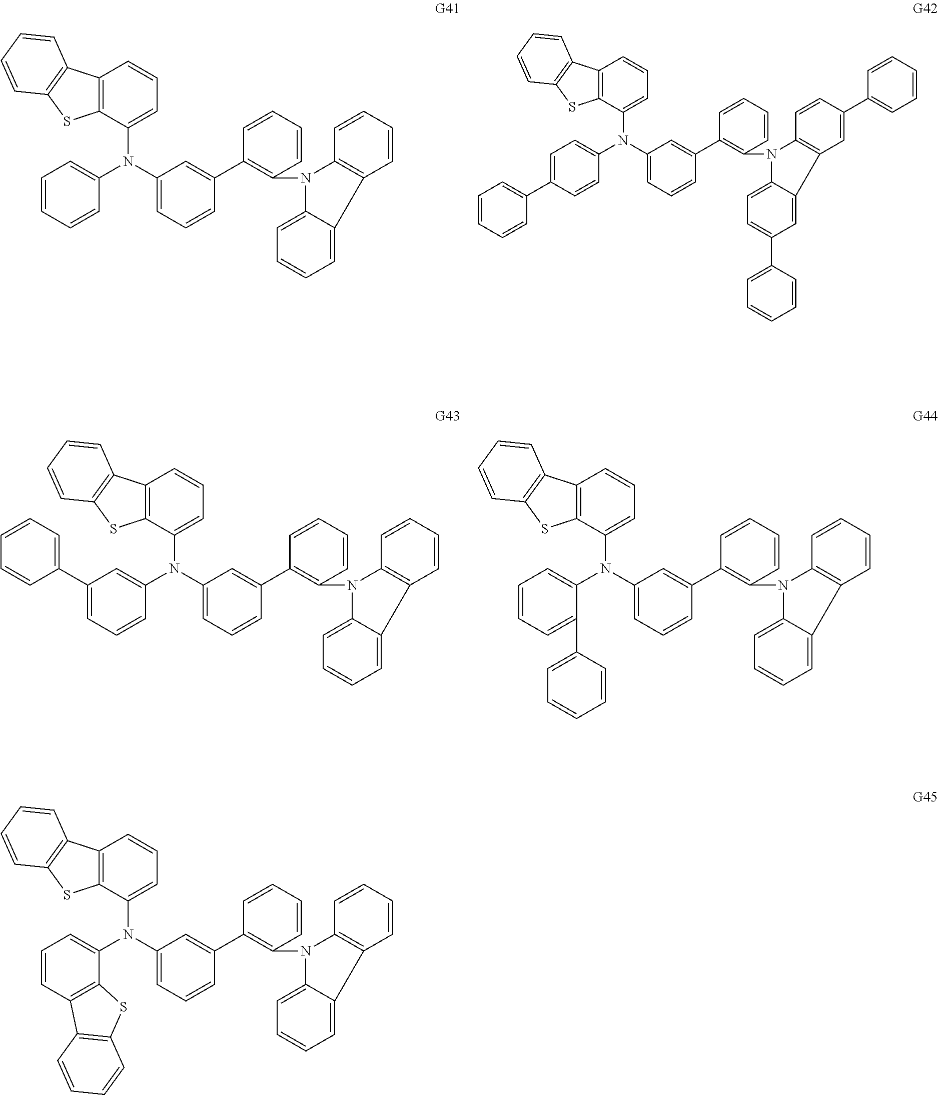 [SMILES: c1ccc(N(c2cccc(-c3ccccc3-n3c4ccccc4c4ccccc43)c2)c2cccc3c2sc2ccccc23)cc1 ; c1ccc(-c2ccc(N(c3cccc(-c4ccccc4-n4c5ccc(-c6ccccc6)cc5c5cc(-c6ccccc6)ccc54)c3)c3cccc4c3sc3ccccc34)cc2)cc1 ; c1ccc(-c2cccc(N(c3cccc(-c4ccccc4-n4c5ccccc5c5ccccc54)c3)c3cccc4c3sc3ccccc34)c2)cc1 ; c1ccc(-c2ccccc2N(c2cccc(-c3ccccc3-n3c4ccccc4c4ccccc43)c2)c2cccc3c2sc2ccccc23)cc1 ; c1cc(-c2ccccc2-n2c3ccccc3c3ccccc32)cc(N(c2cccc3c2sc2ccccc23)c2cccc3c2sc2ccccc23)c1] 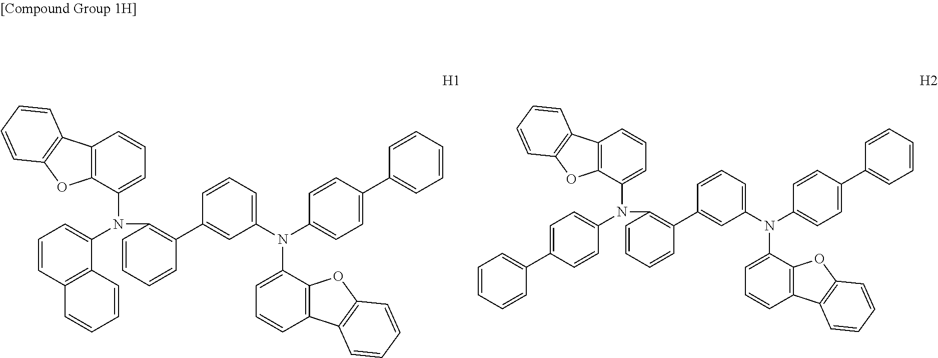 [SMILES: c1ccc(-c2ccc(N(c3cccc(-c4ccccc4N(c4cccc5ccccc45)c4cccc5c4oc4ccccc45)c3)c3cccc4c3oc3ccccc34)cc2)cc1 ; c1ccc(-c2ccc(N(c3cccc(-c4ccccc4N(c4ccc(-c5ccccc5)cc4)c4cccc5c4oc4ccccc45)c3)c3cccc4c3oc3ccccc34)cc2)cc1] 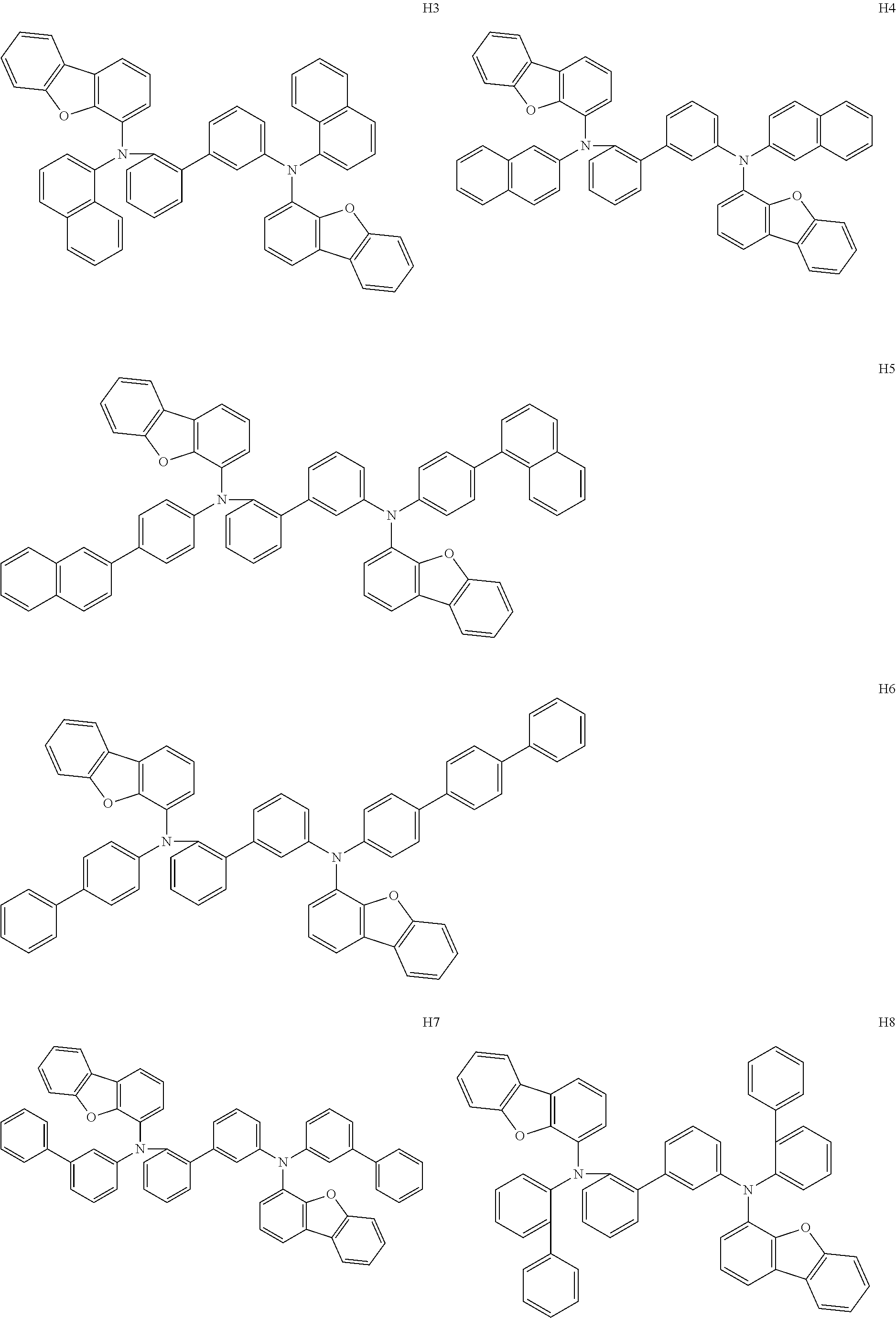 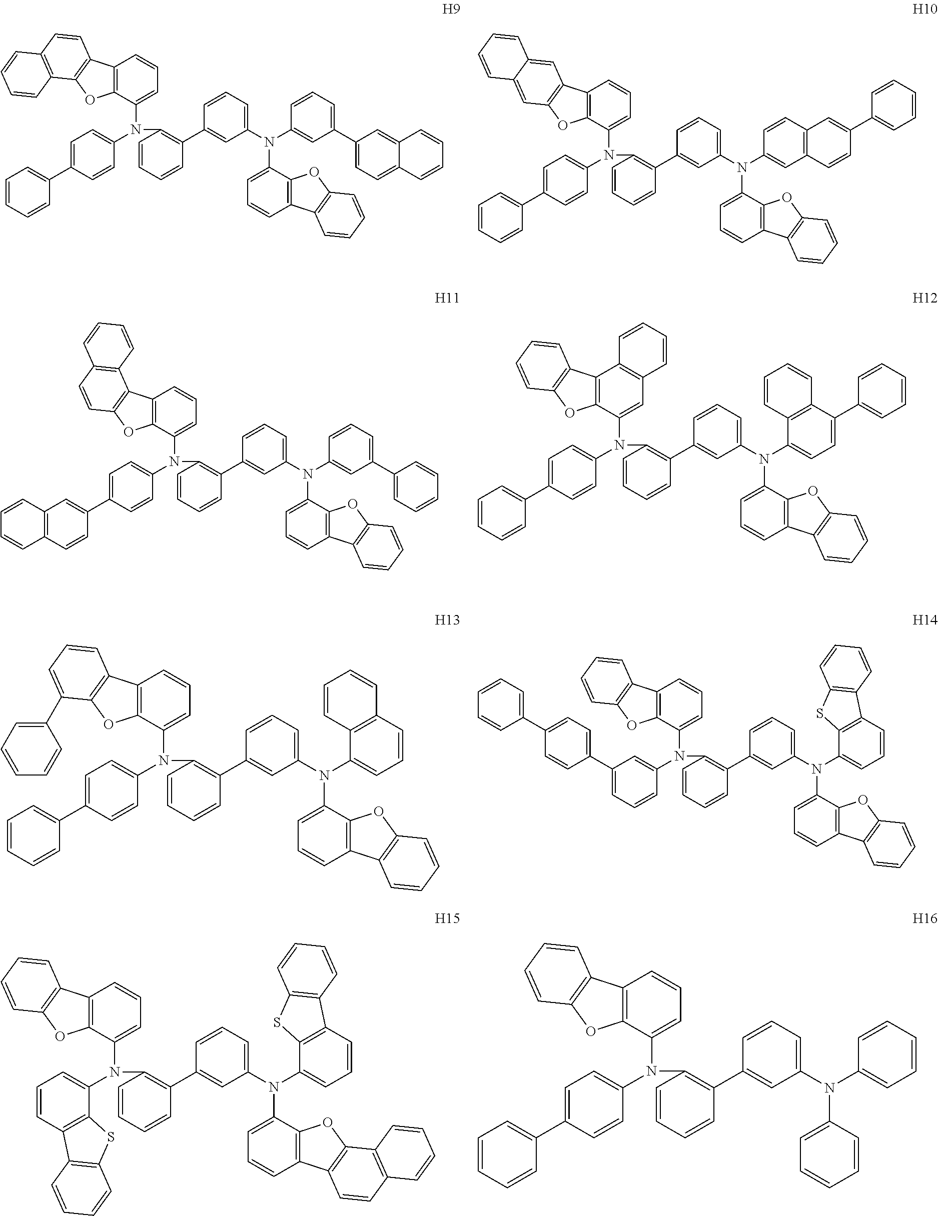 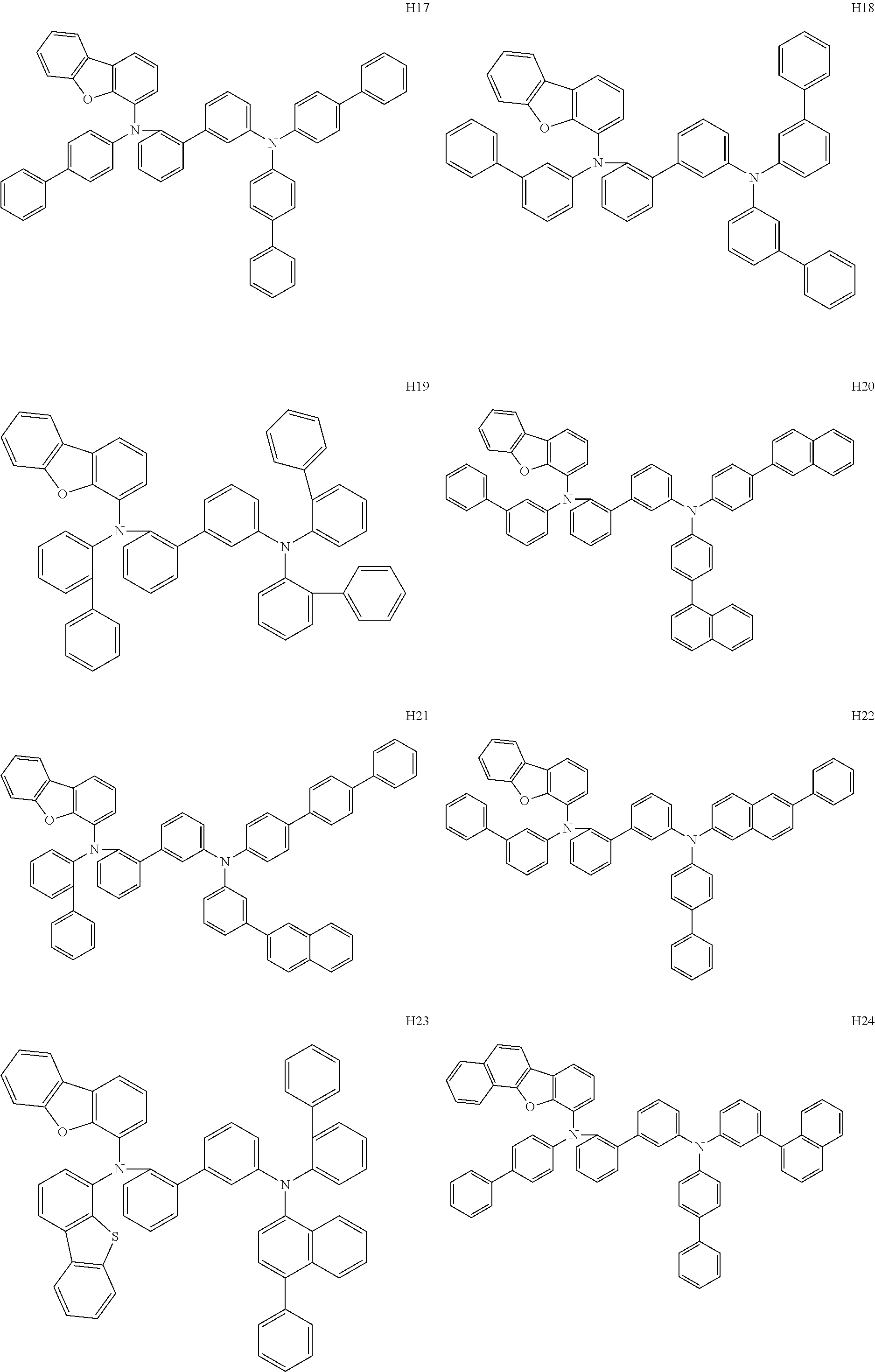 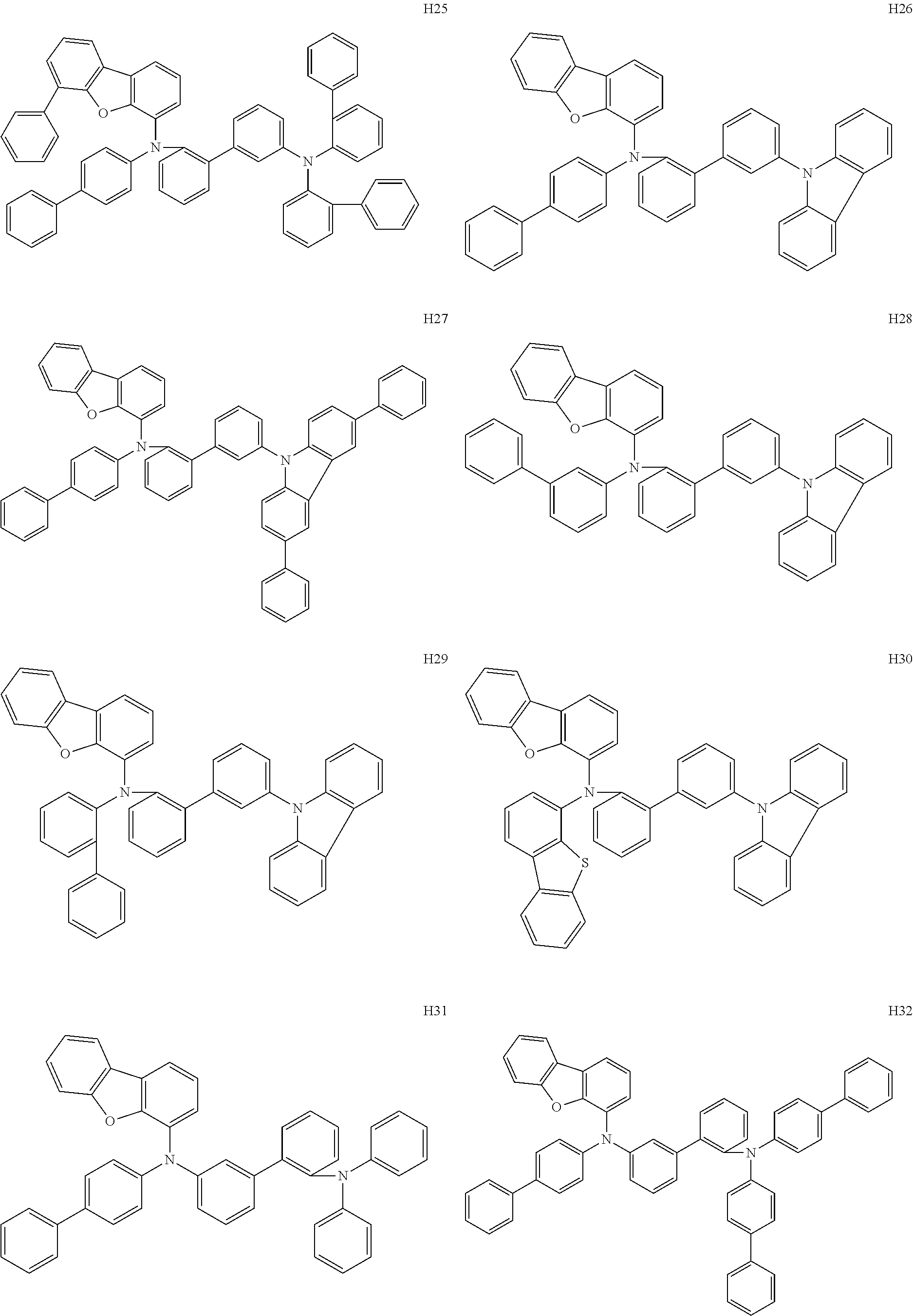 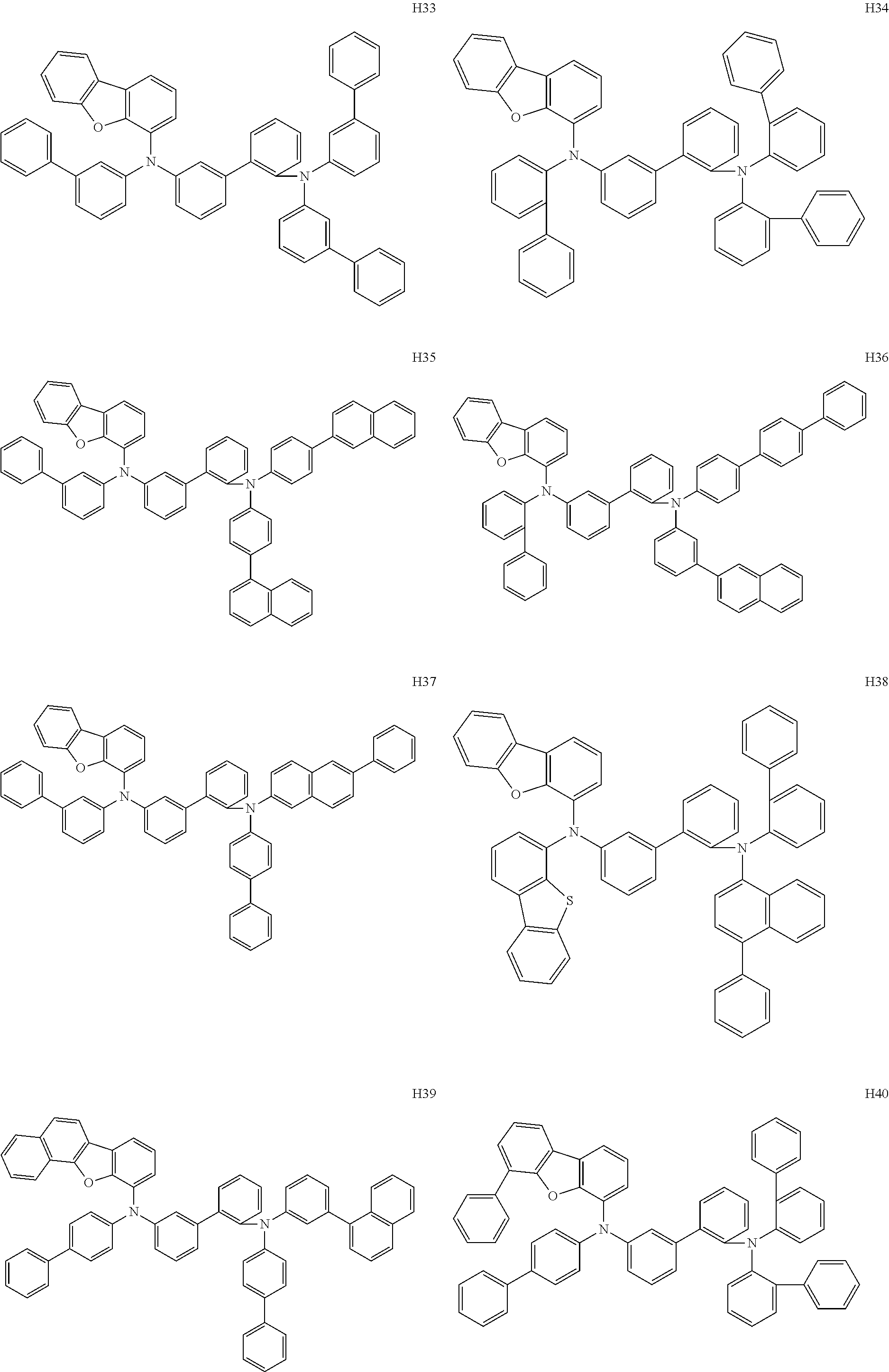 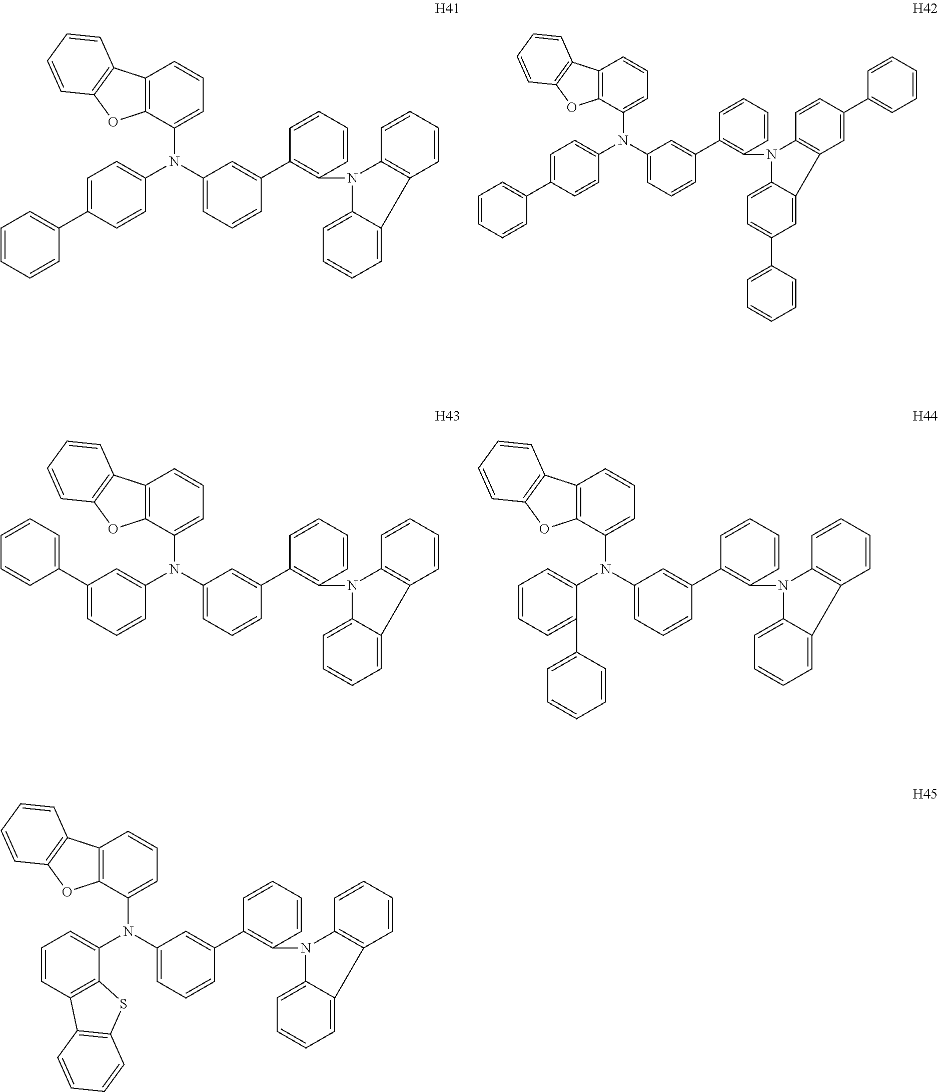 [SMILES: c1ccc(-c2ccc(N(c3cccc(-c4ccccc4-n4c5ccccc5c5ccccc54)c3)c3cccc4c3oc3ccccc34)cc2)cc1 ; c1ccc(-c2ccc(N(c3cccc(-c4ccccc4-n4c5ccc(-c6ccccc6)cc5c5cc(-c6ccccc6)ccc54)c3)c3cccc4c3oc3ccccc34)cc2)cc1 ; c1ccc(-c2cccc(N(c3cccc(-c4ccccc4-n4c5ccccc5c5ccccc54)c3)c3cccc4c3oc3ccccc34)c2)cc1 ; c1ccc(-c2ccccc2N(c2cccc(-c3ccccc3-n3c4ccccc4c4ccccc43)c2)c2cccc3c2oc2ccccc23)cc1 ; c1cc(-c2ccccc2-n2c3ccccc3c3ccccc32)cc(N(c2cccc3c2oc2ccccc23)c2cccc3c2sc2ccccc23)c1] 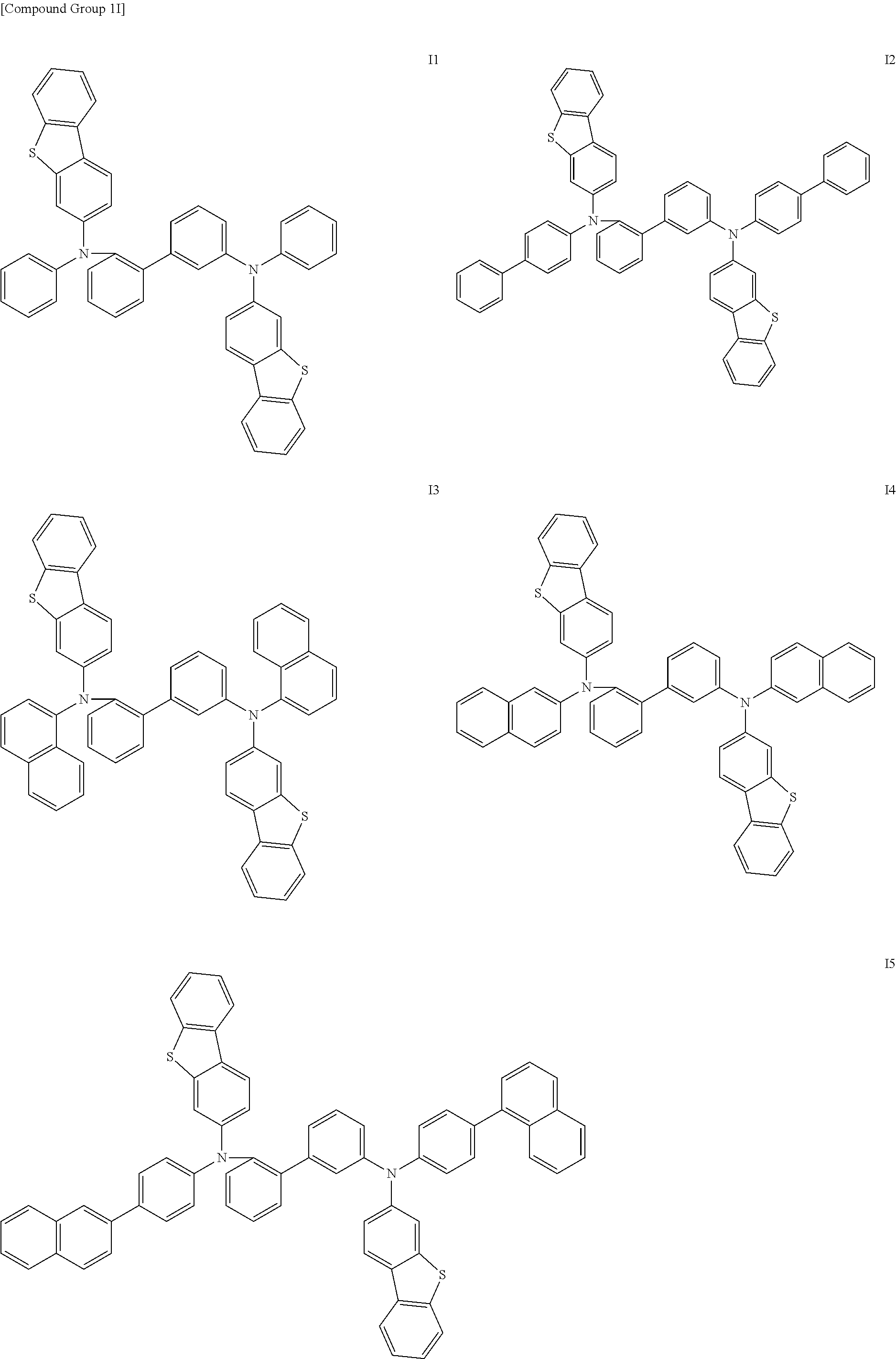 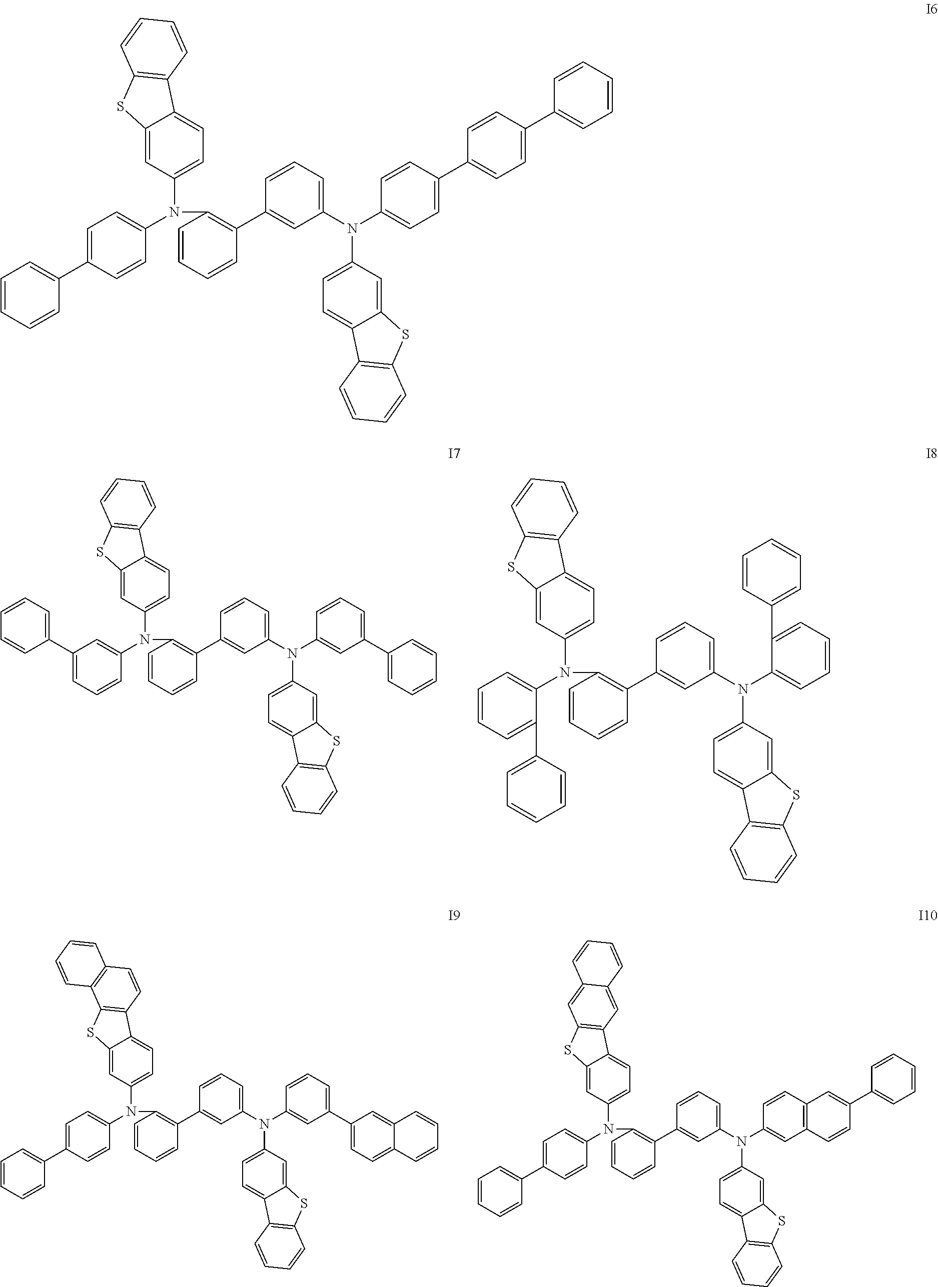 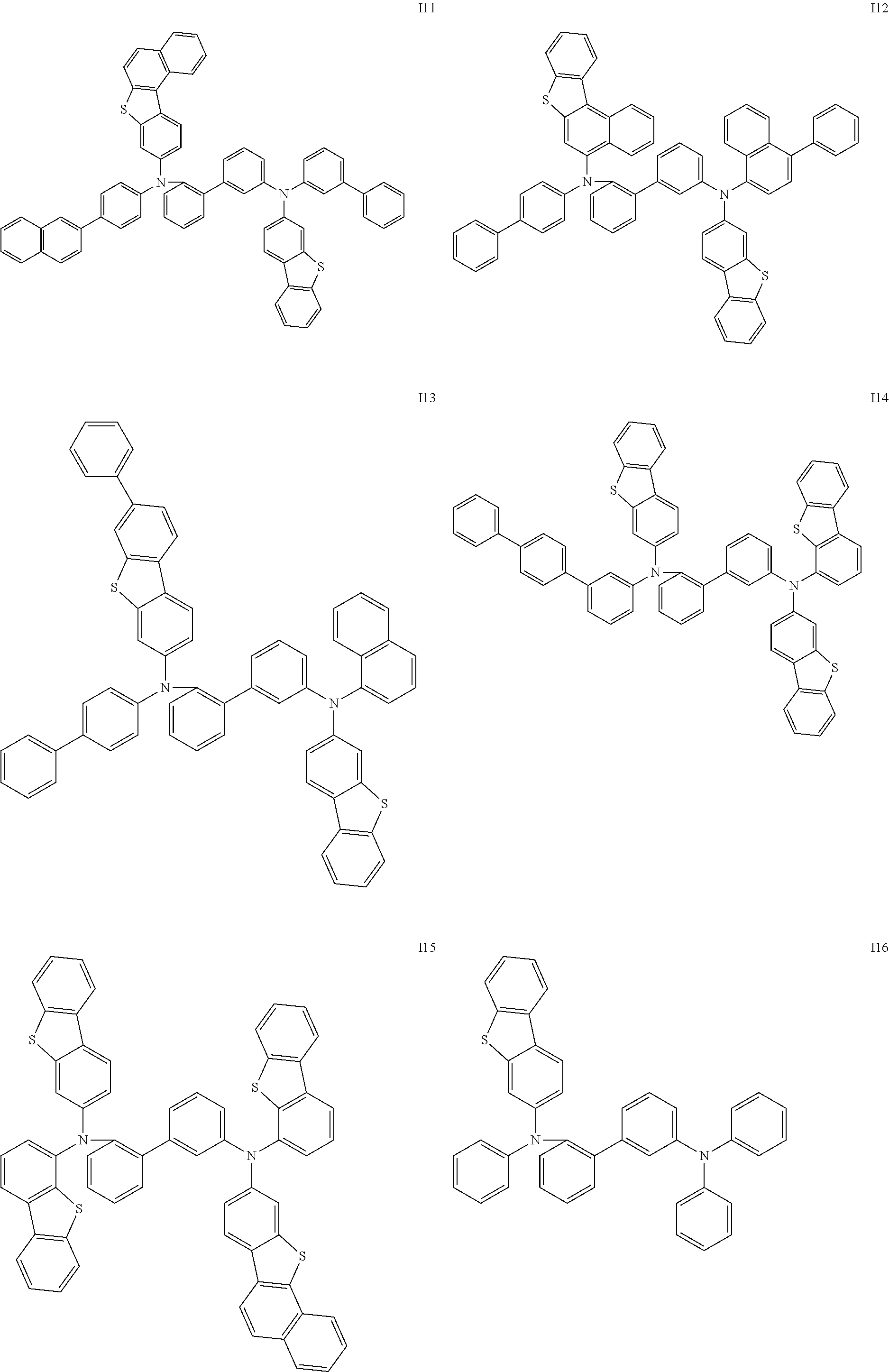 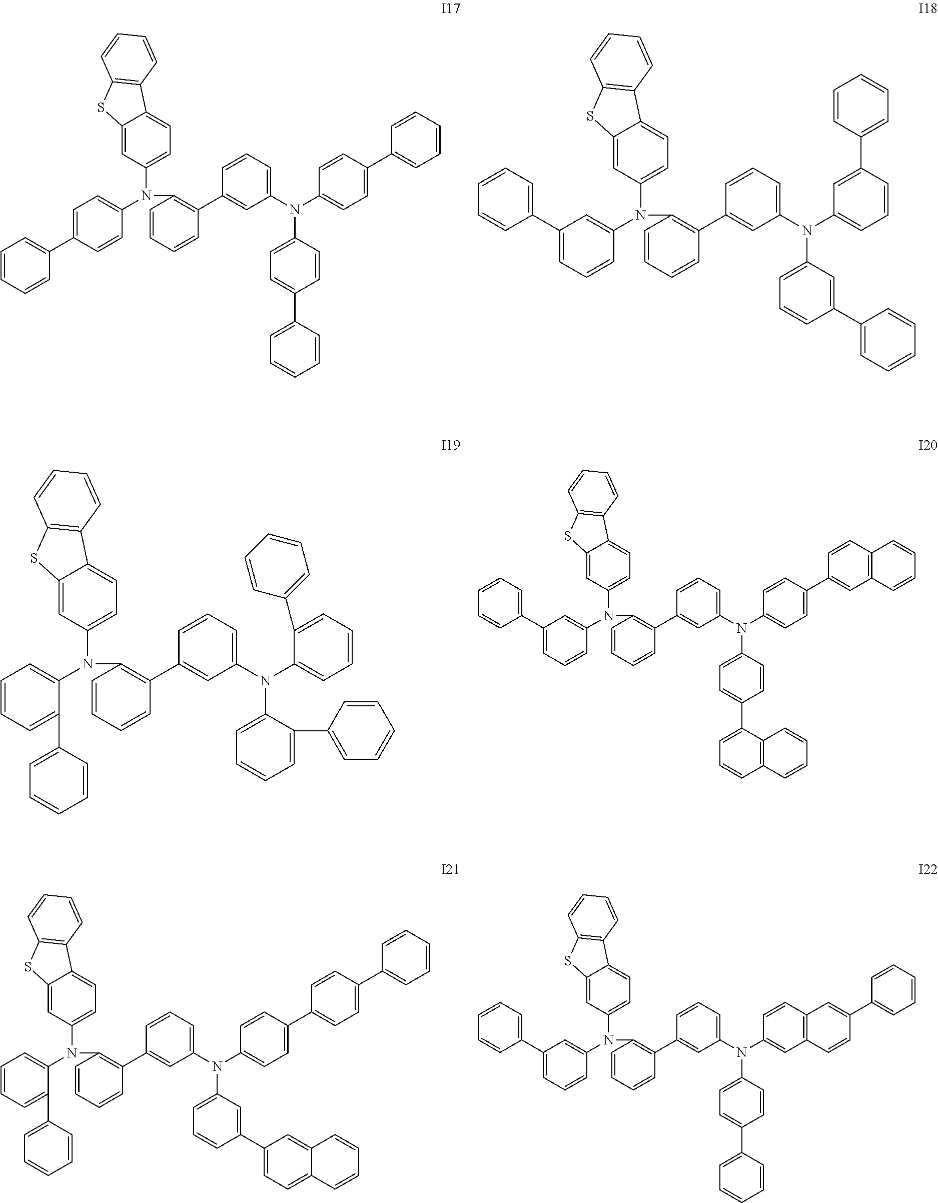 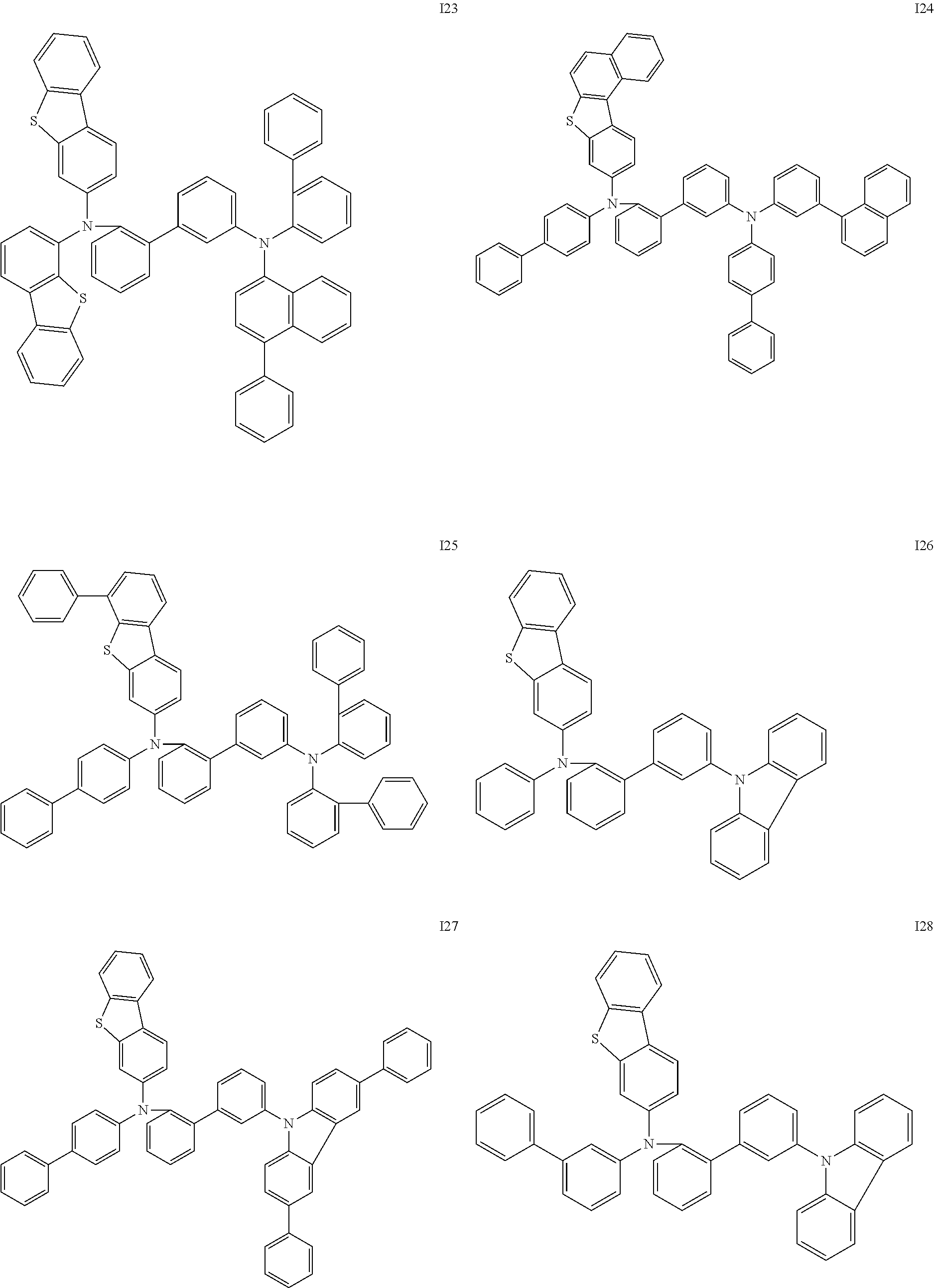 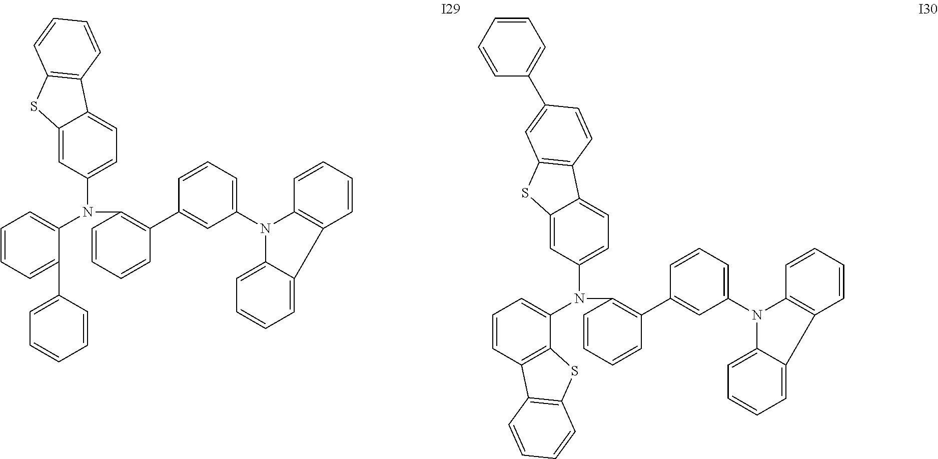 [SMILES: c1ccc(-c2ccccc2N(c2ccc3c(c2)sc2ccccc23)c2ccccc2-c2cccc(-n3c4ccccc4c4ccccc43)c2)cc1 ; c1ccc(-c2ccc3c(c2)sc2cc(N(c4ccccc4-c4cccc(-n5c6ccccc6c6ccccc65)c4)c4cccc5c4sc4ccccc45)ccc23)cc1] 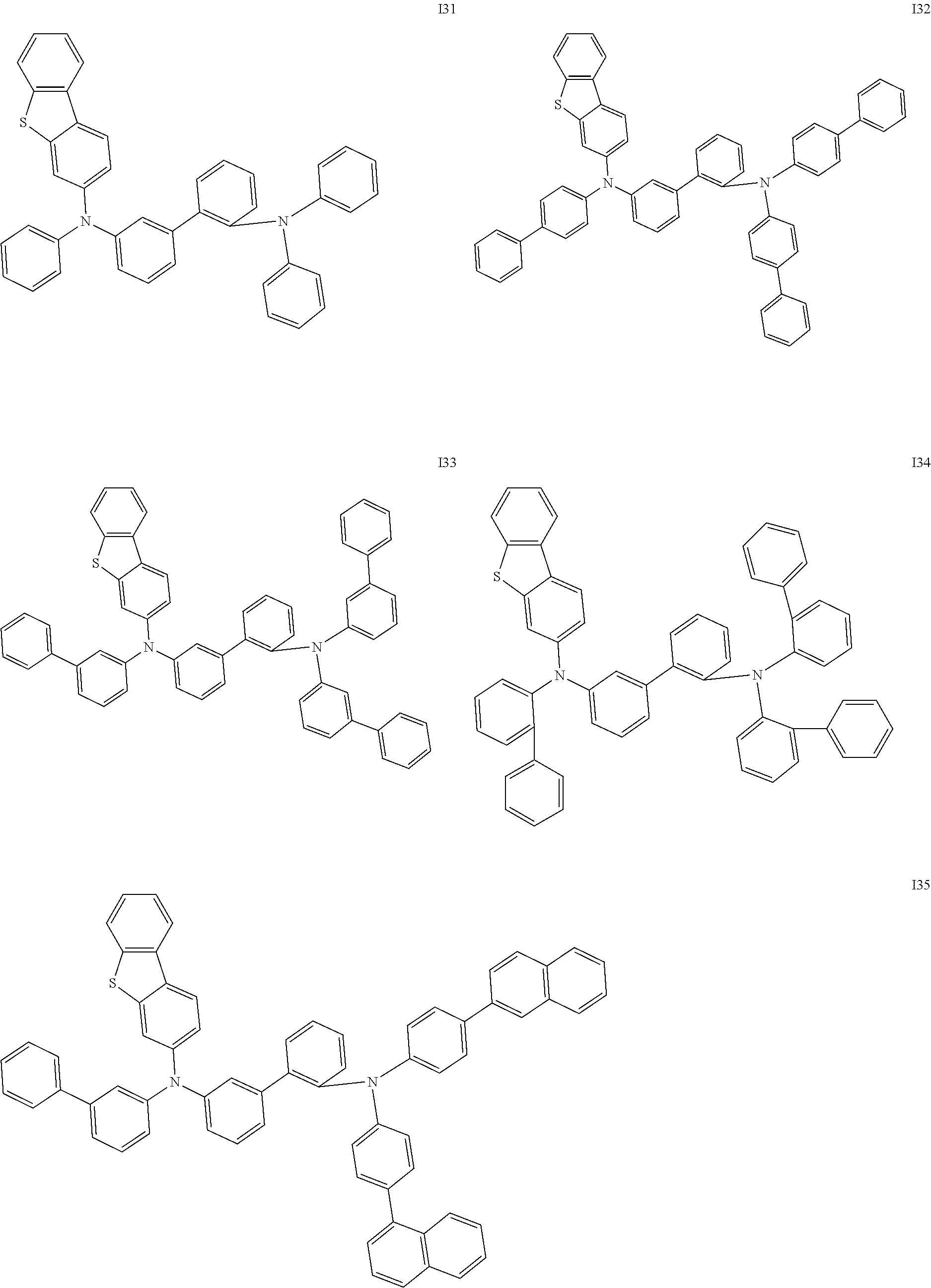 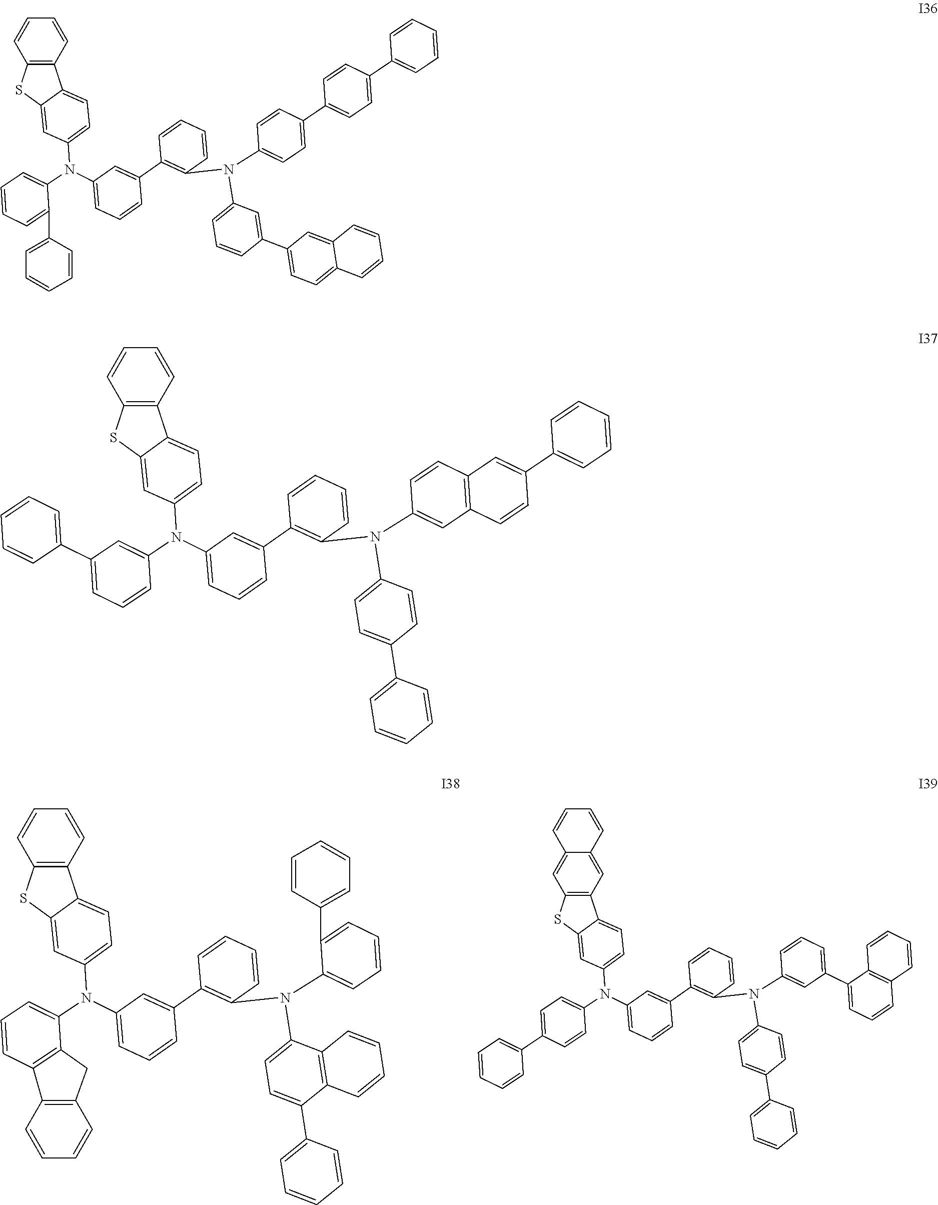 [SMILES: c1ccc(-c2ccc(-c3ccc(N(c4cccc(-c5ccc6ccccc6c5)c4)c4ccccc4-c4cccc(N(c5ccc6c(c5)sc5ccccc56)c5ccccc5-c5ccccc5)c4)cc3)cc2)cc1 ; c1ccc(-c2ccc(N(c3ccc4cc(-c5ccccc5)ccc4c3)c3ccccc3-c3cccc(N(c4cccc(-c5ccccc5)c4)c4ccc5c(c4)sc4ccccc45)c3)cc2)cc1 ; c1ccc(-c2ccccc2N(c2ccccc2-c2cccc(N(c3ccc4c(c3)sc3ccccc34)c3cccc4c3Cc3ccccc3-4)c2)c2ccc(-c3ccccc3)c3ccccc23)cc1 ; c1ccc(-c2ccc(N(c3cccc(-c4ccccc4N(c4ccc(-c5ccccc5)cc4)c4cccc(-c5cccc6ccccc56)c4)c3)c3ccc4c(c3)sc3cc5ccccc5cc34)cc2)cc1] 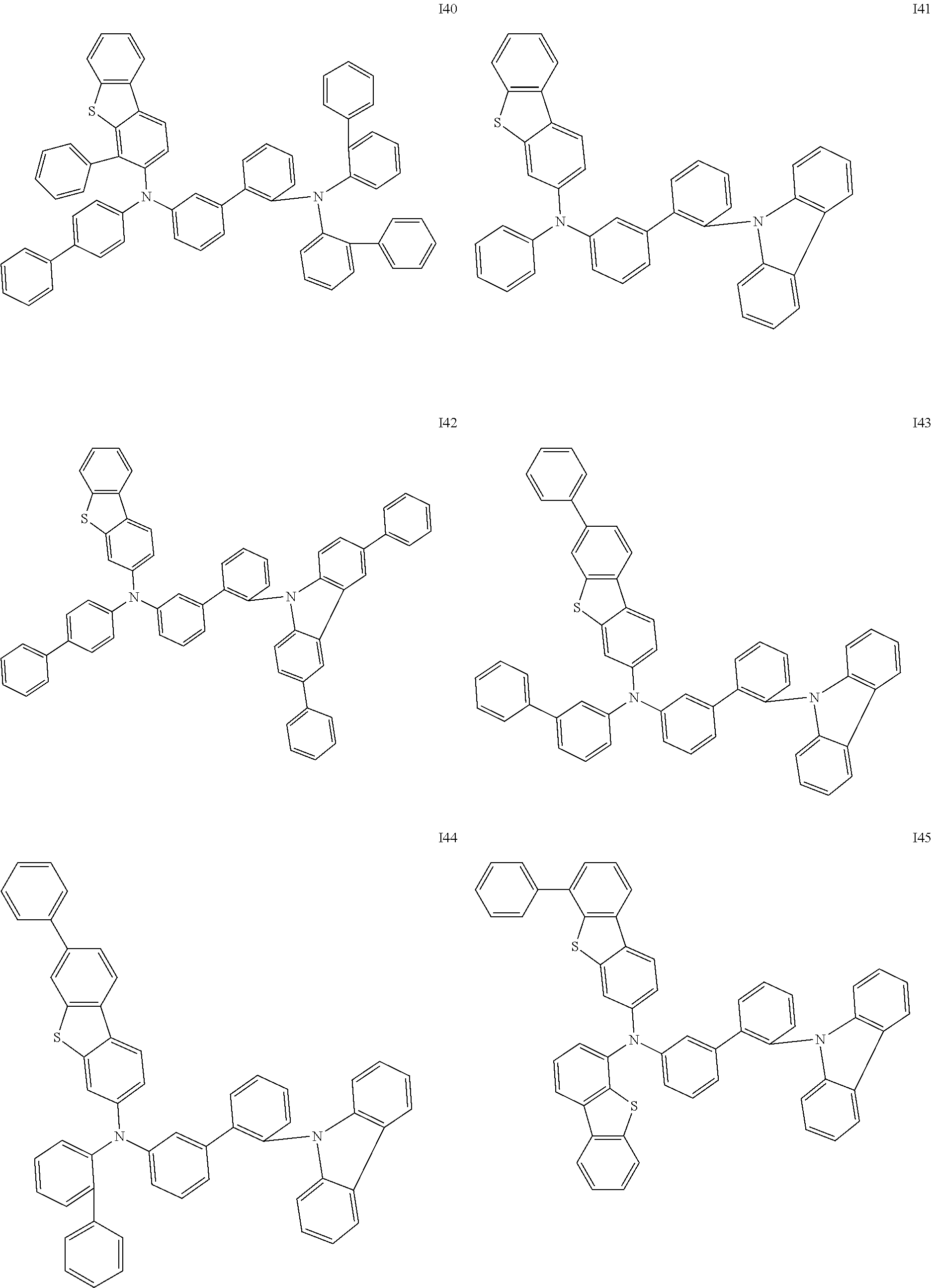 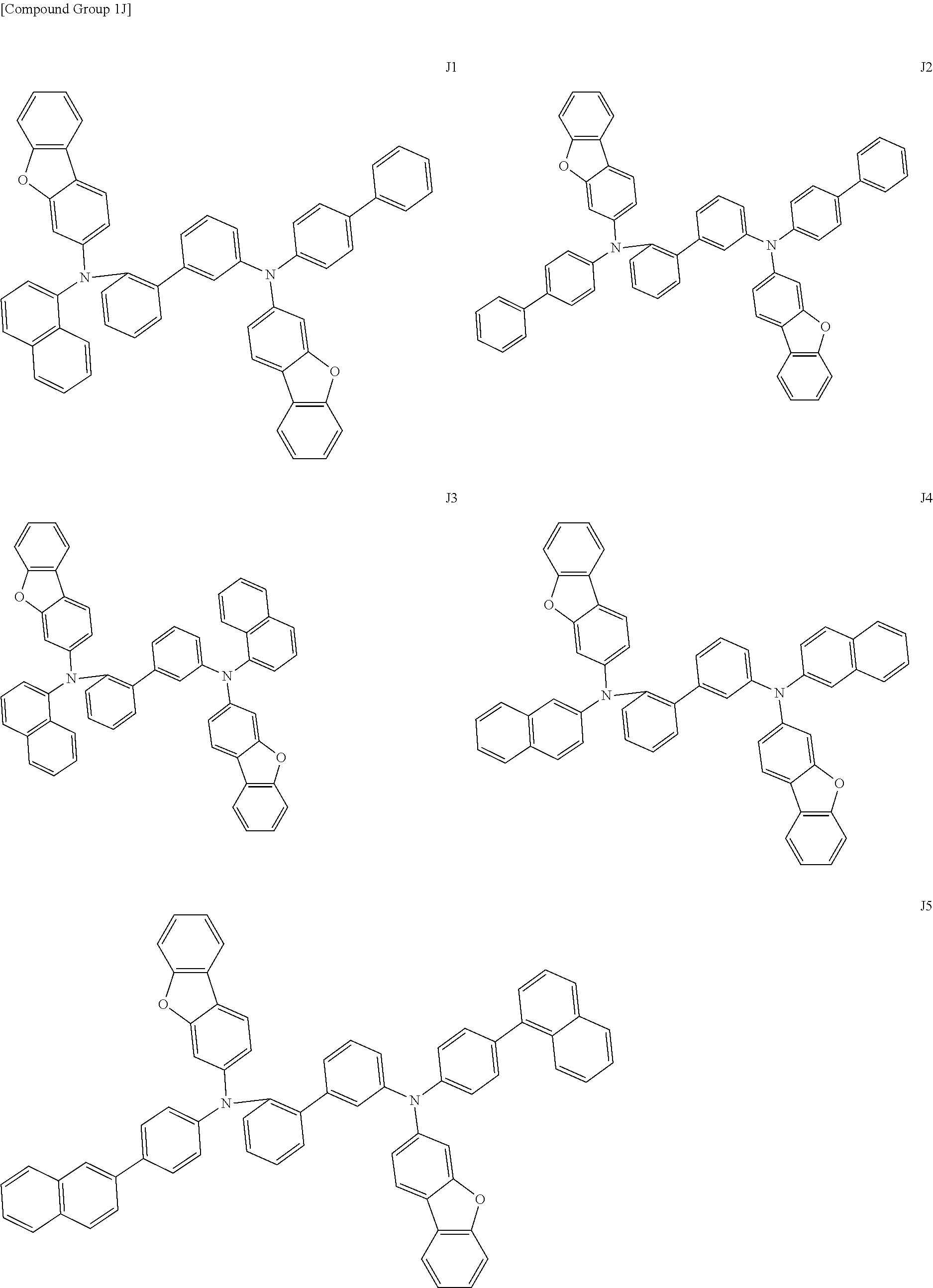 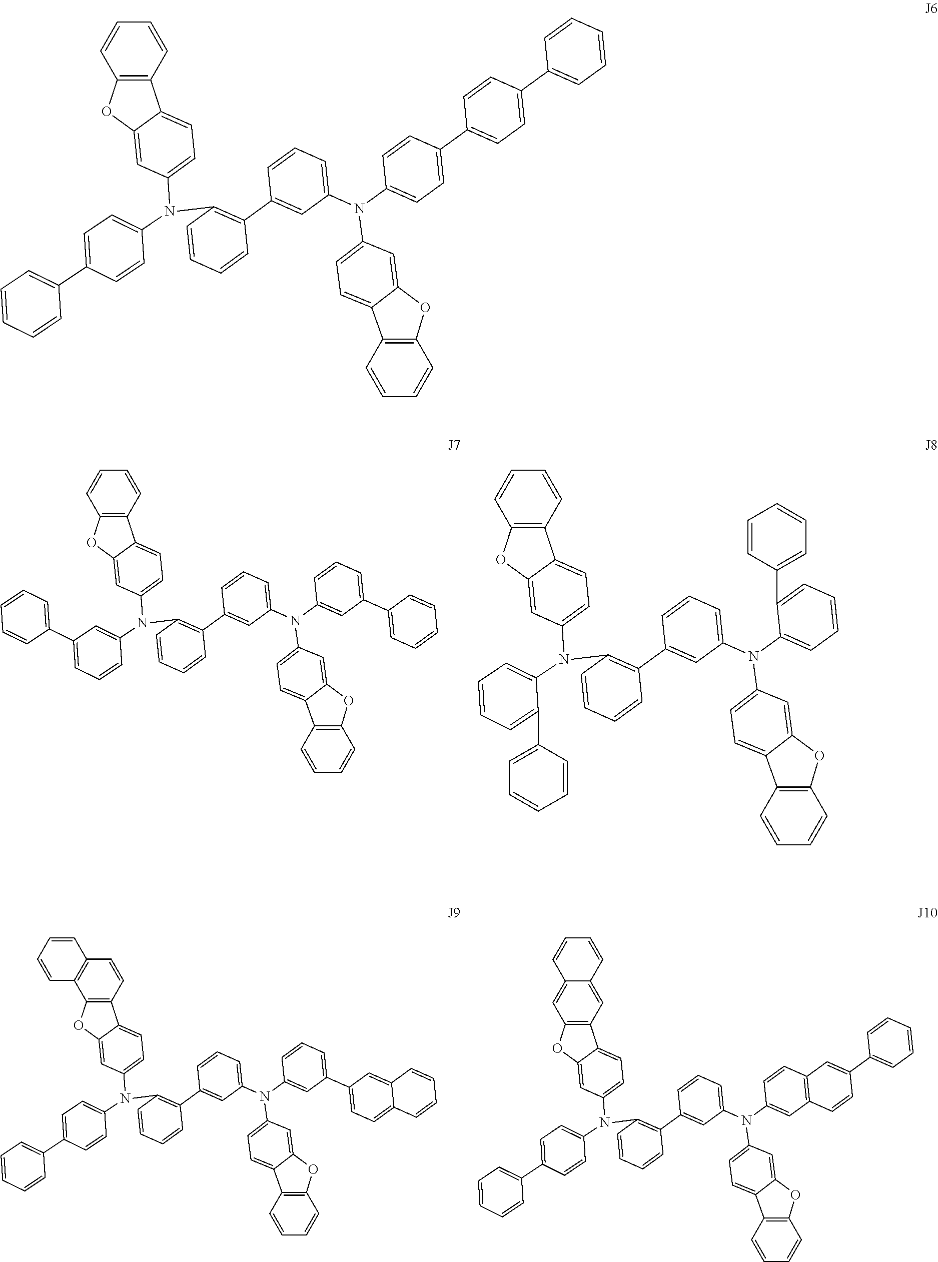 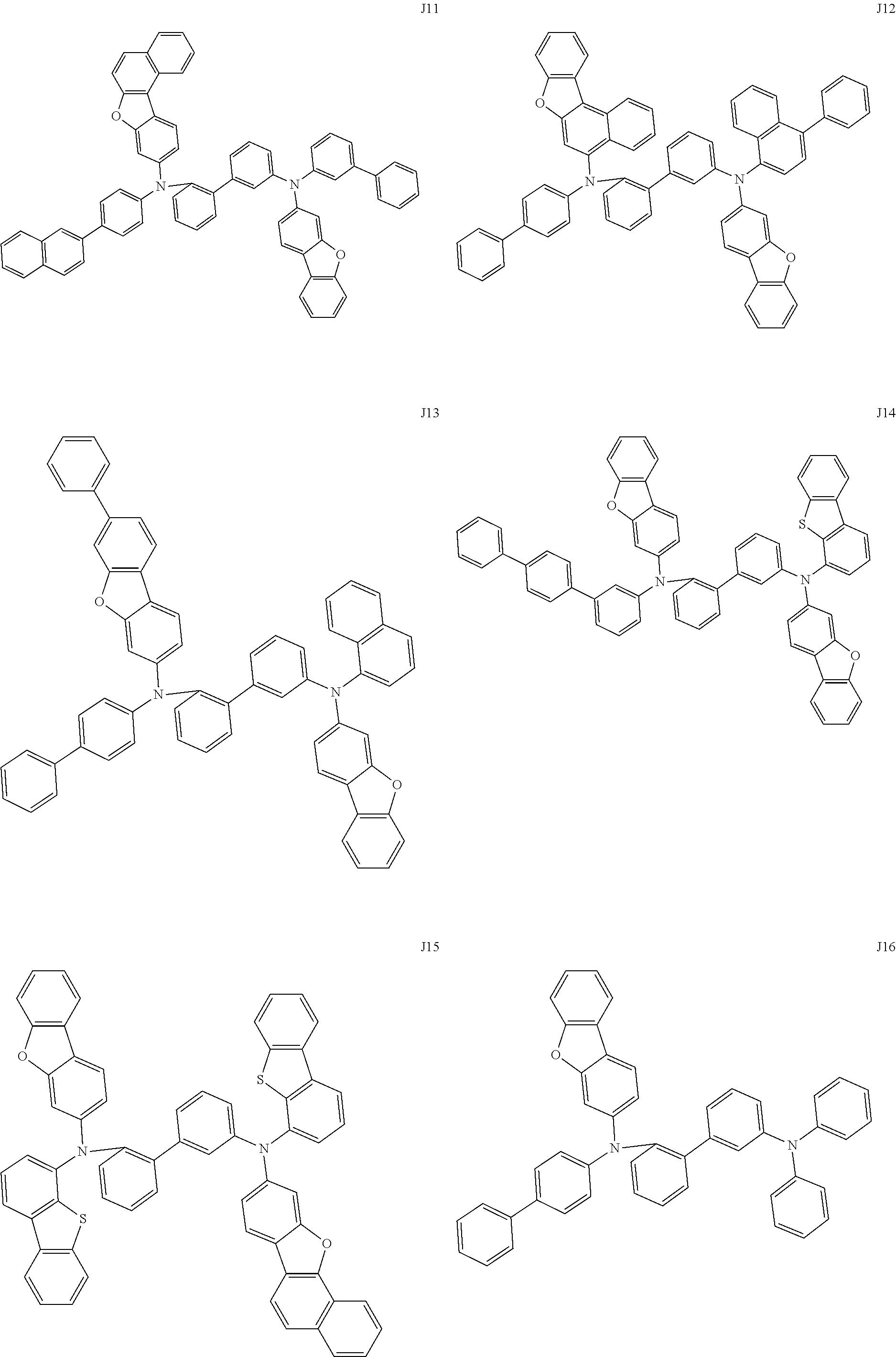 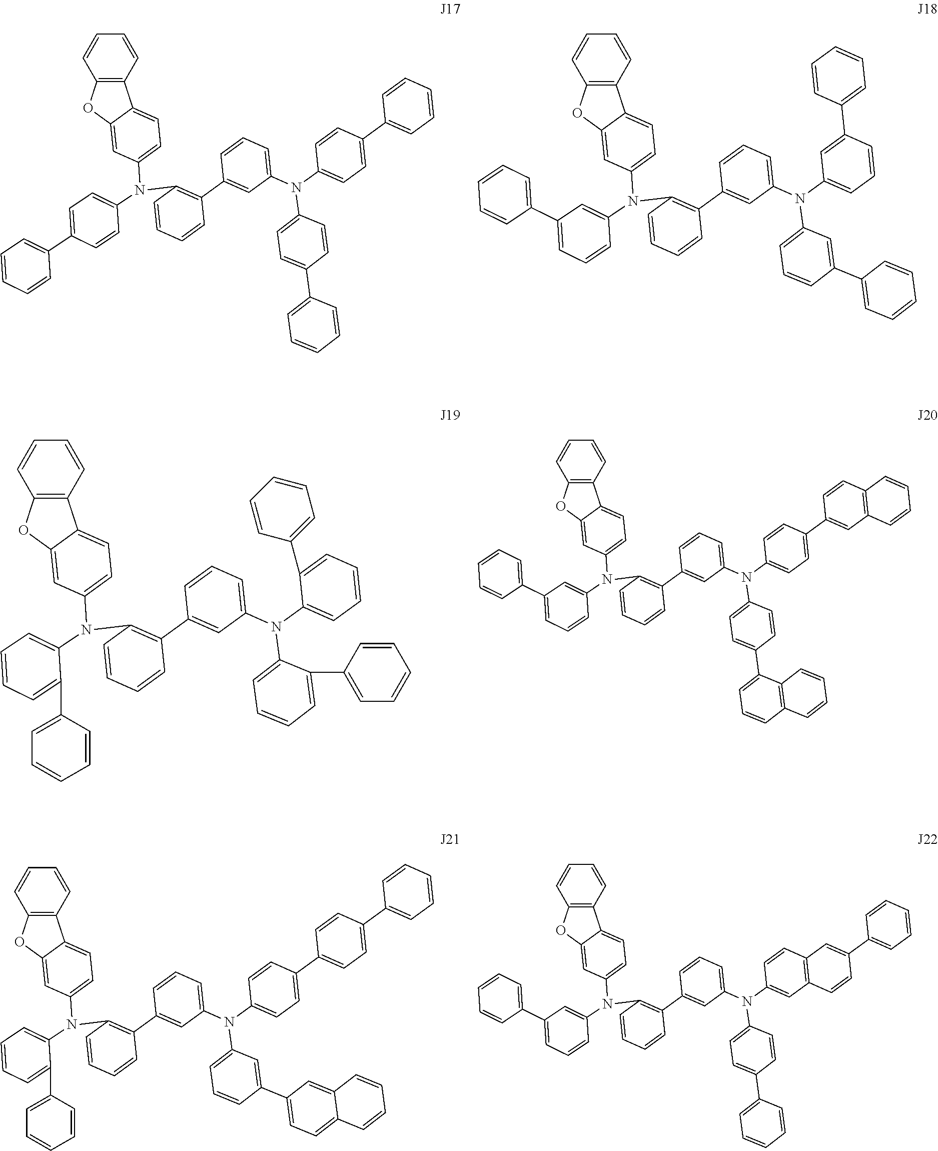 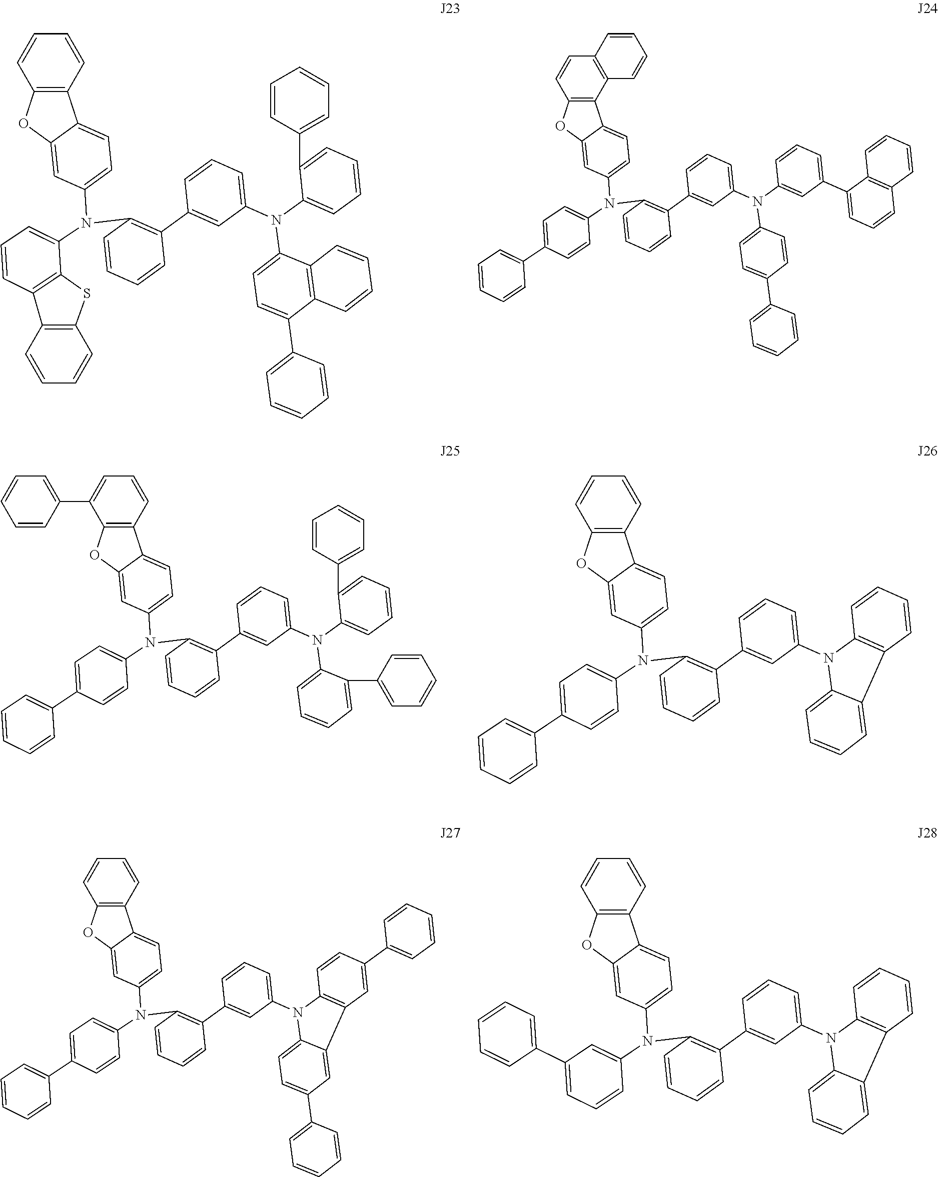 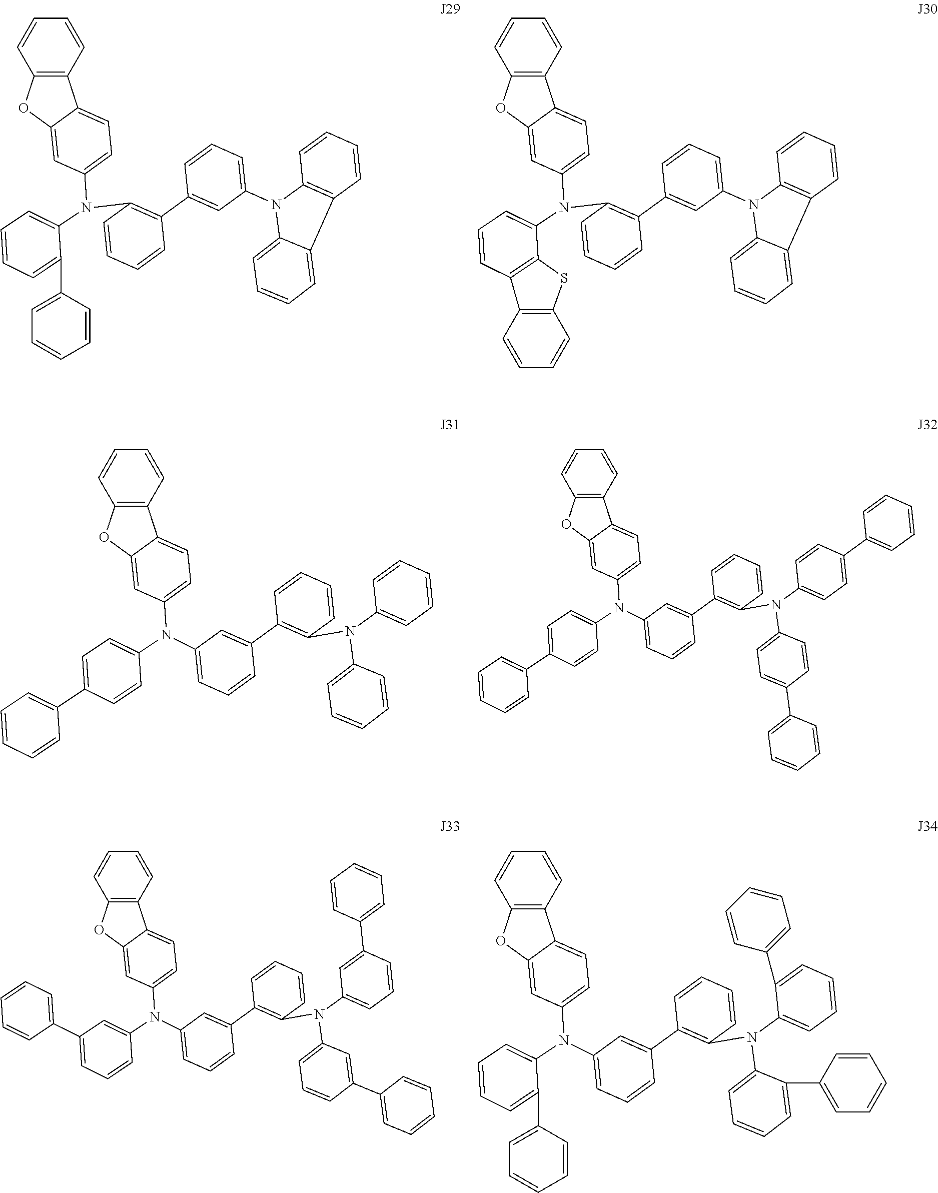 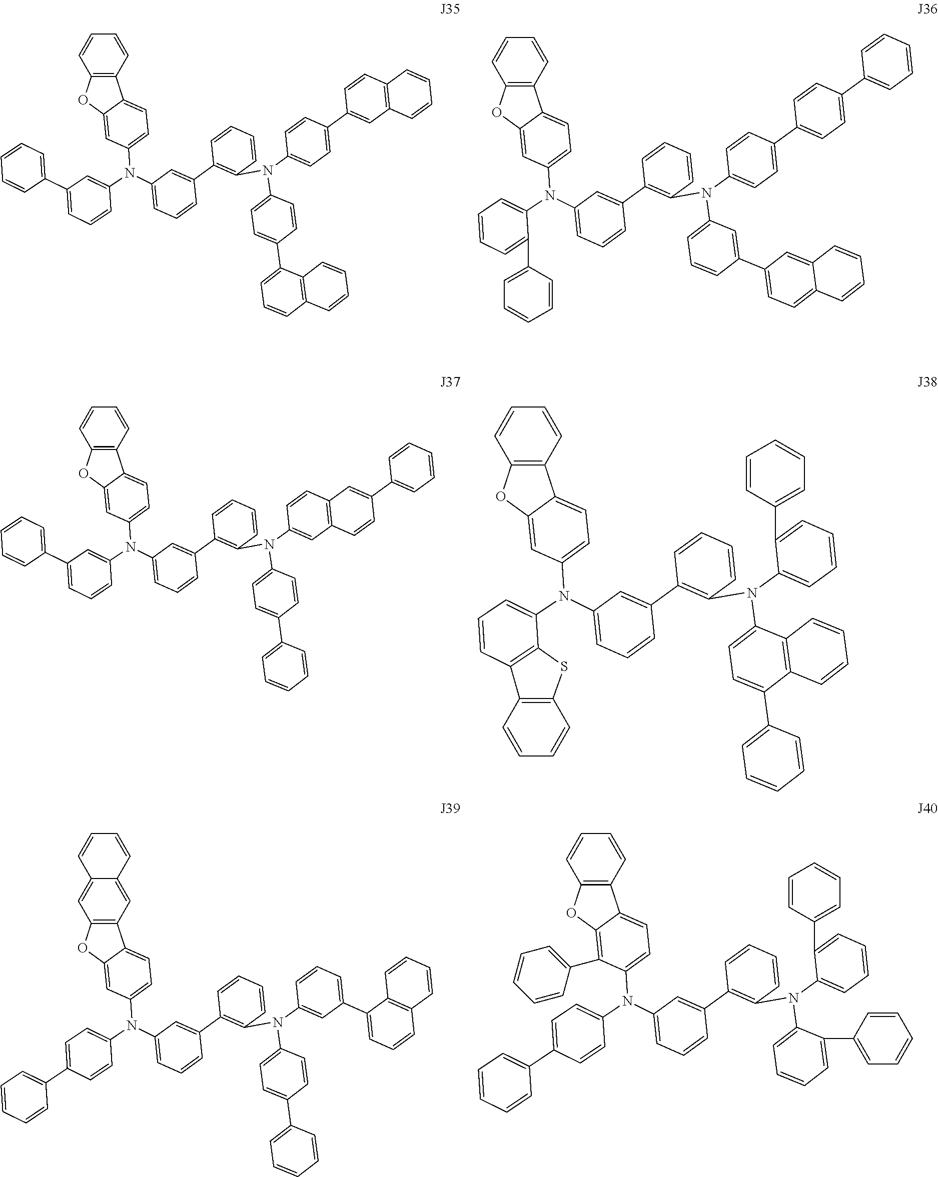 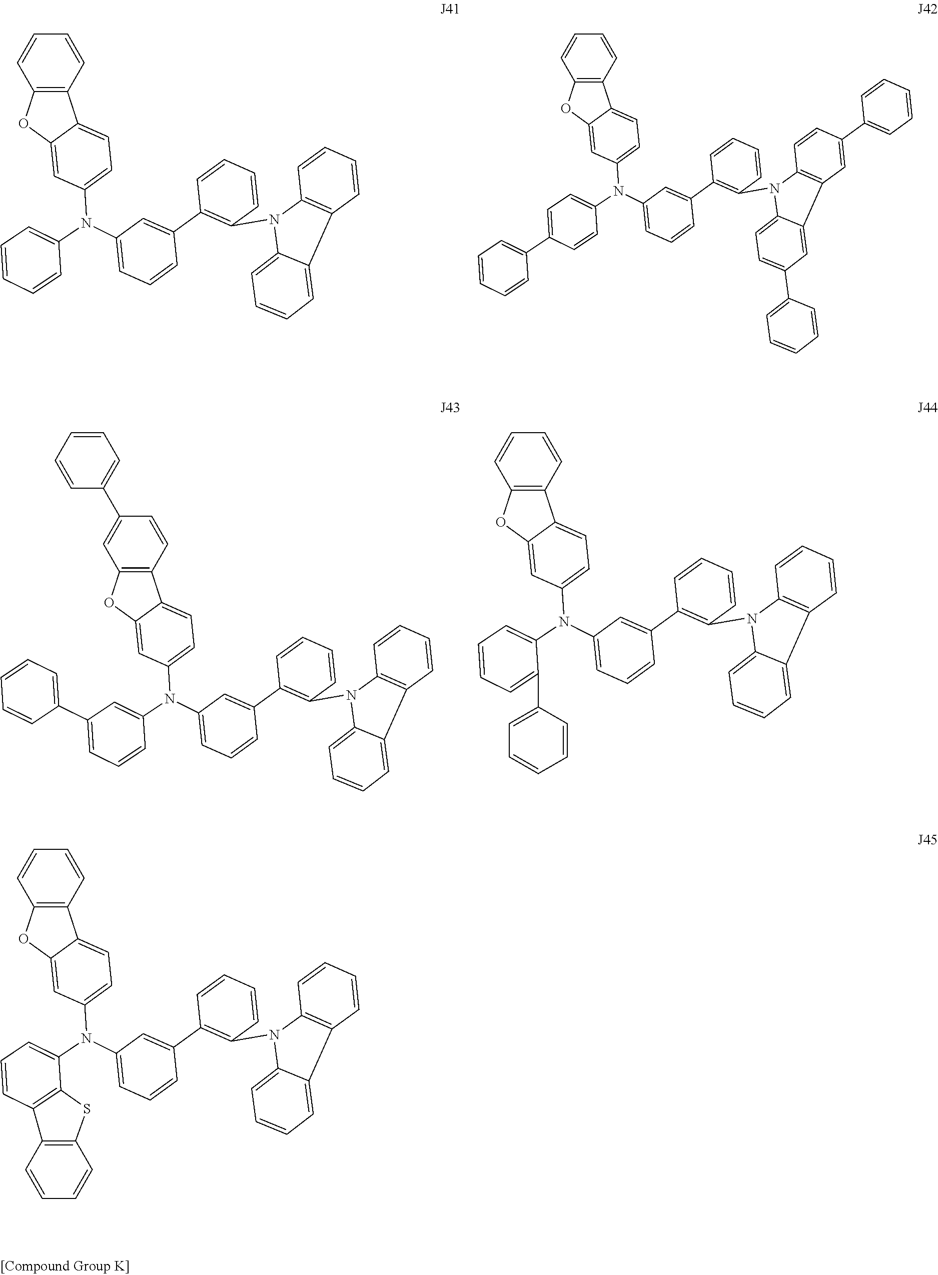 [SMILES: c1ccc(N(c2cccc(-c3ccccc3-n3c4ccccc4c4ccccc43)c2)c2ccc3c(c2)oc2ccccc23)cc1 ; c1ccc(-c2ccc(N(c3cccc(-c4ccccc4-n4c5ccc(-c6ccccc6)cc5c5cc(-c6ccccc6)ccc54)c3)c3ccc4c(c3)oc3ccccc34)cc2)cc1 ; c1ccc(-c2cccc(N(c3cccc(-c4ccccc4-n4c5ccccc5c5ccccc54)c3)c3ccc4c(c3)oc3cc(-c5ccccc5)ccc34)c2)cc1 ; c1ccc(-c2ccccc2N(c2cccc(-c3ccccc3-n3c4ccccc4c4ccccc43)c2)c2ccc3c(c2)oc2ccccc23)cc1 ; c1cc(-c2ccccc2-n2c3ccccc3c3ccccc32)cc(N(c2ccc3c(c2)oc2ccccc23)c2cccc3c2sc2ccccc23)c1] 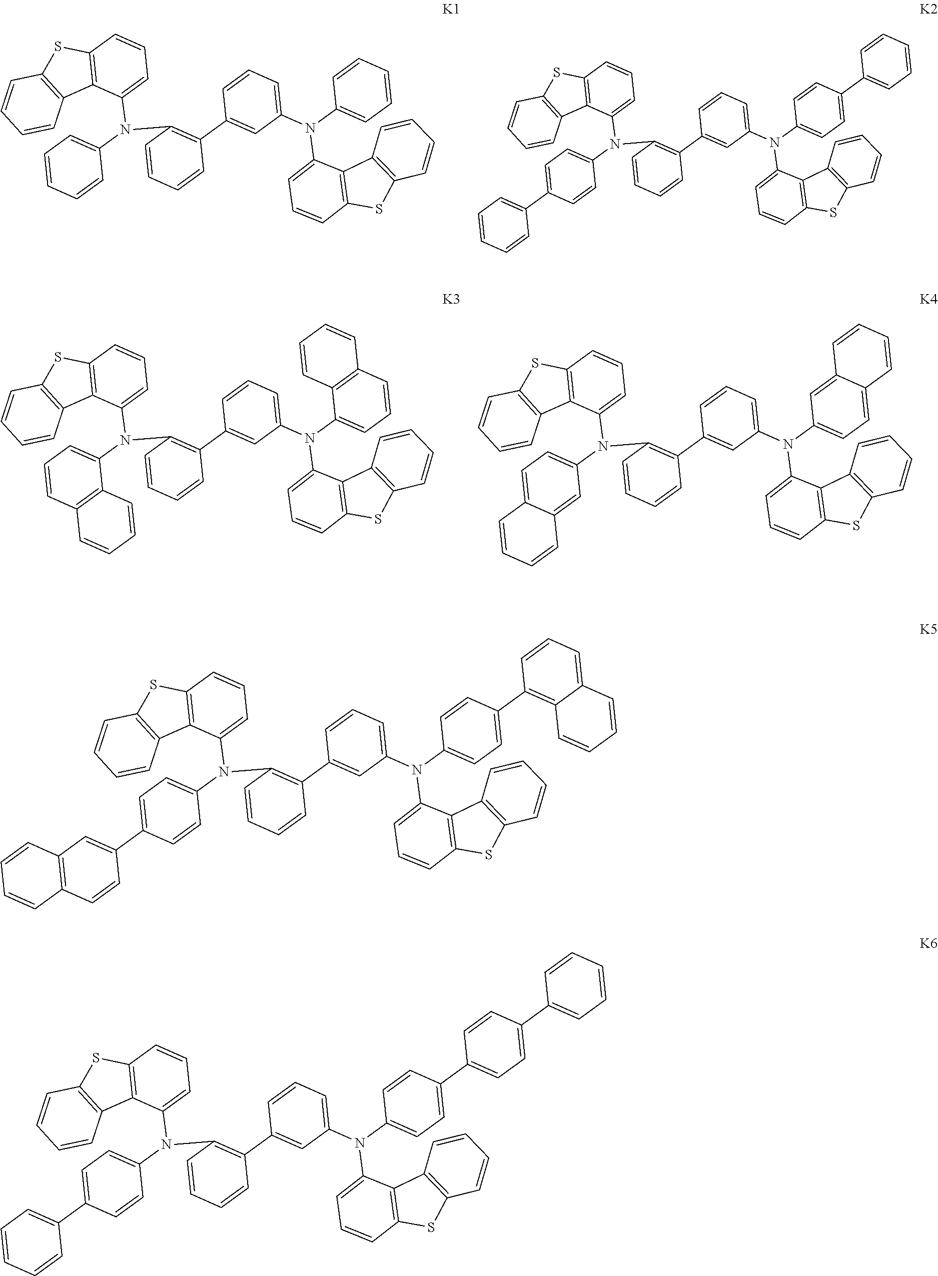 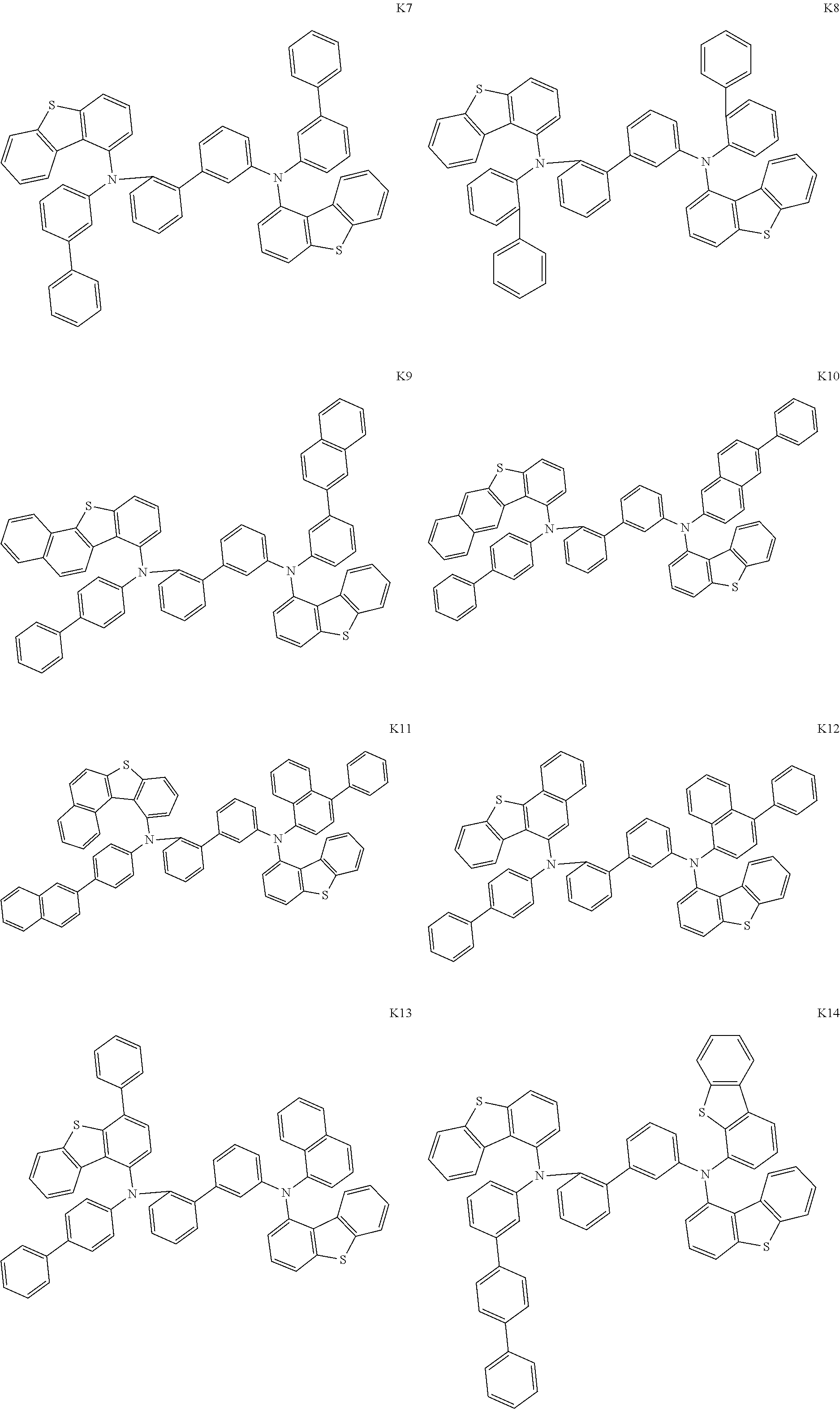 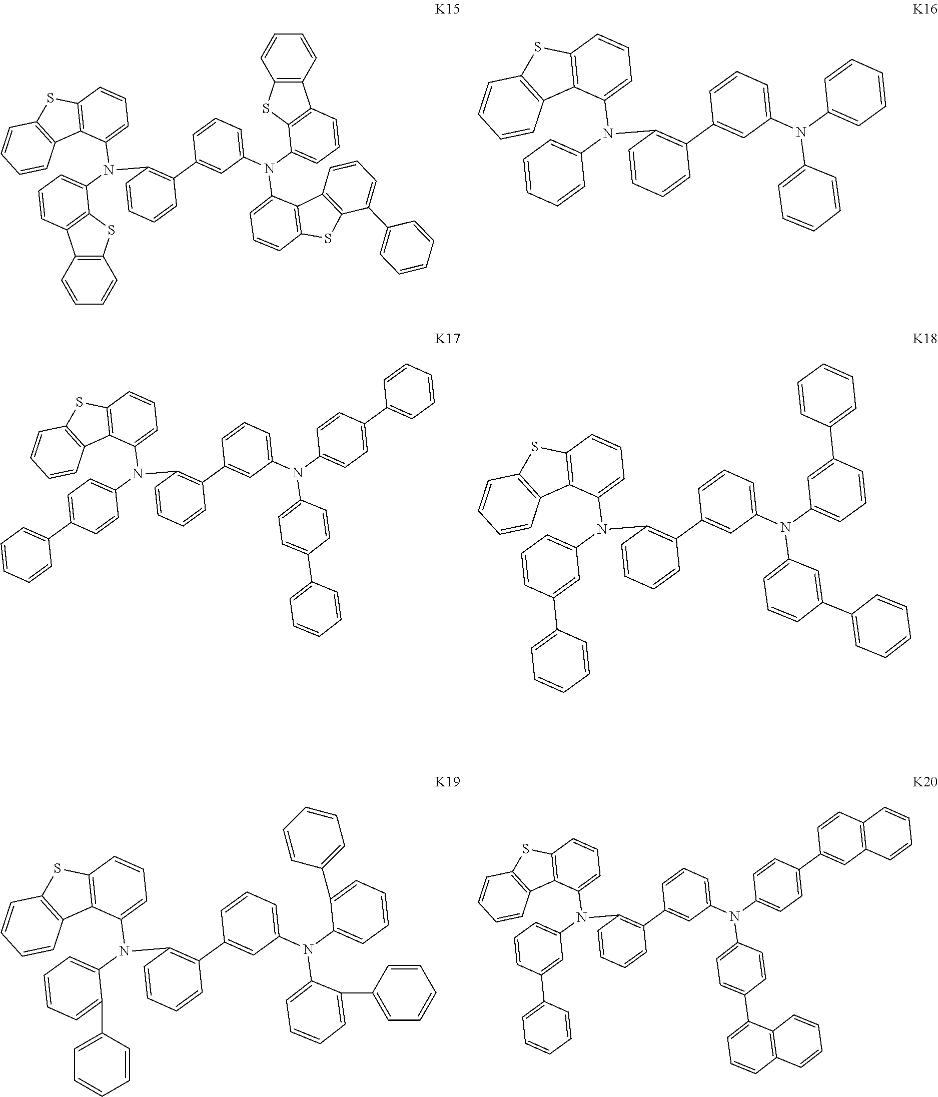 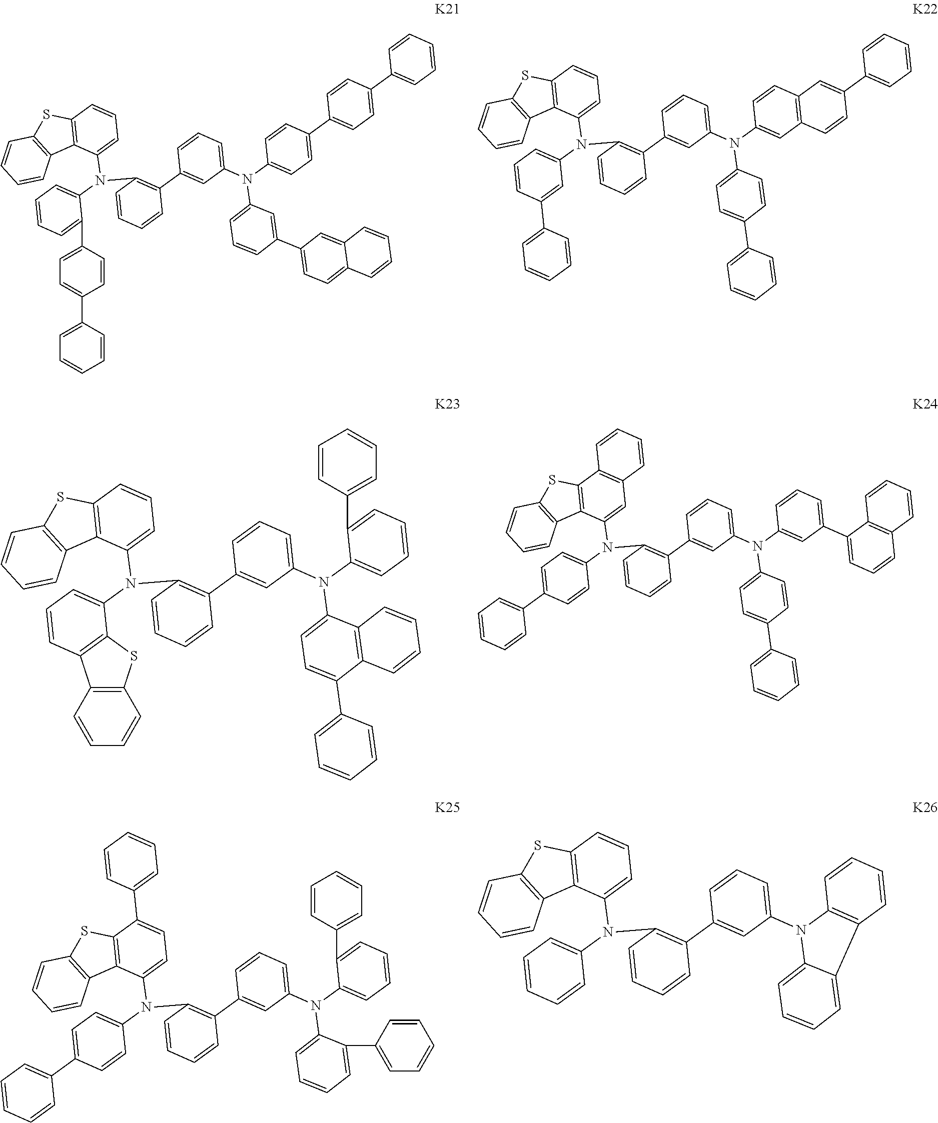 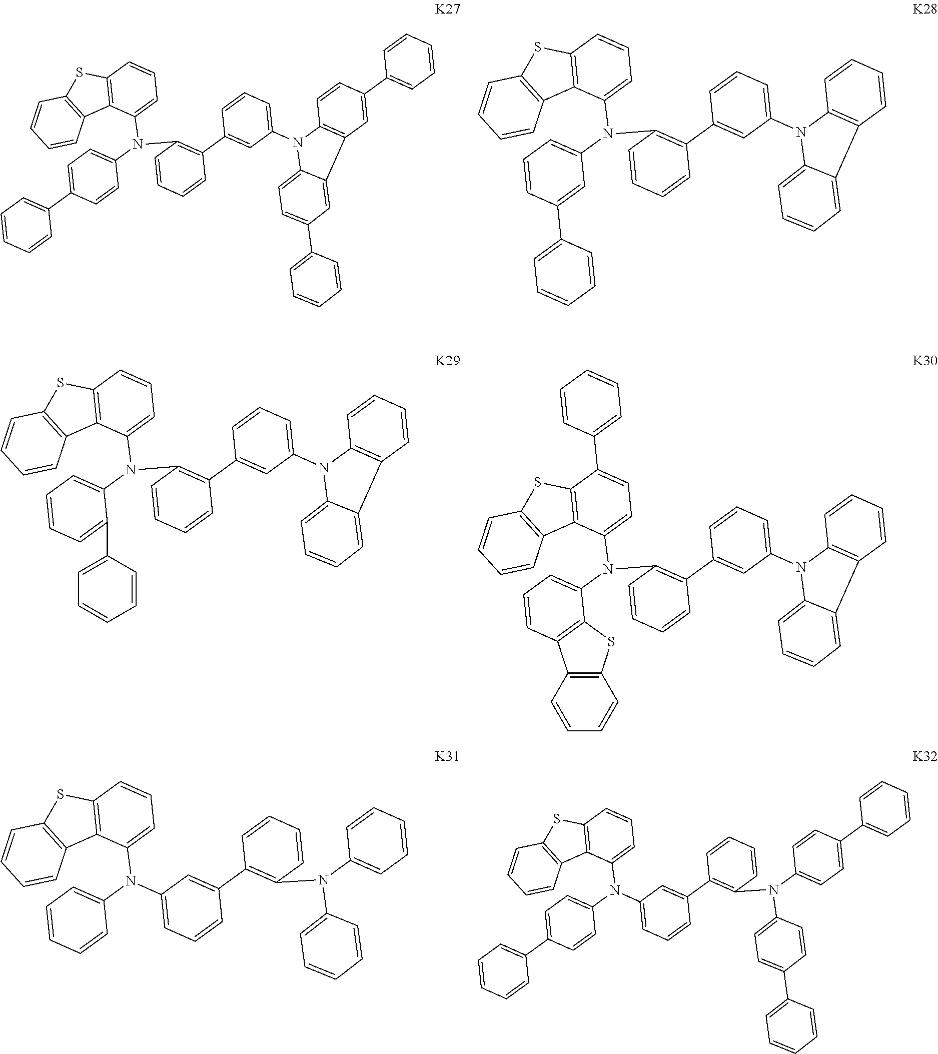 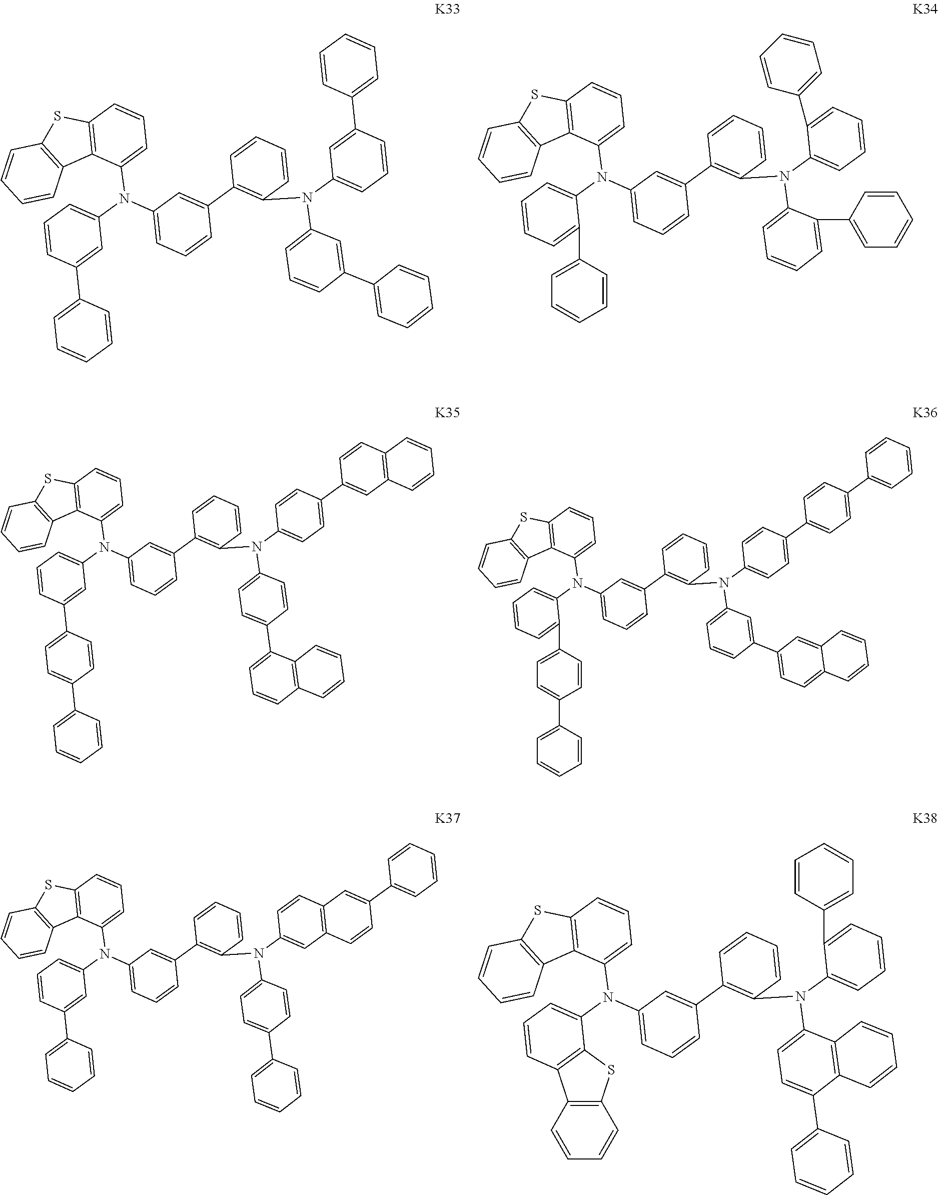 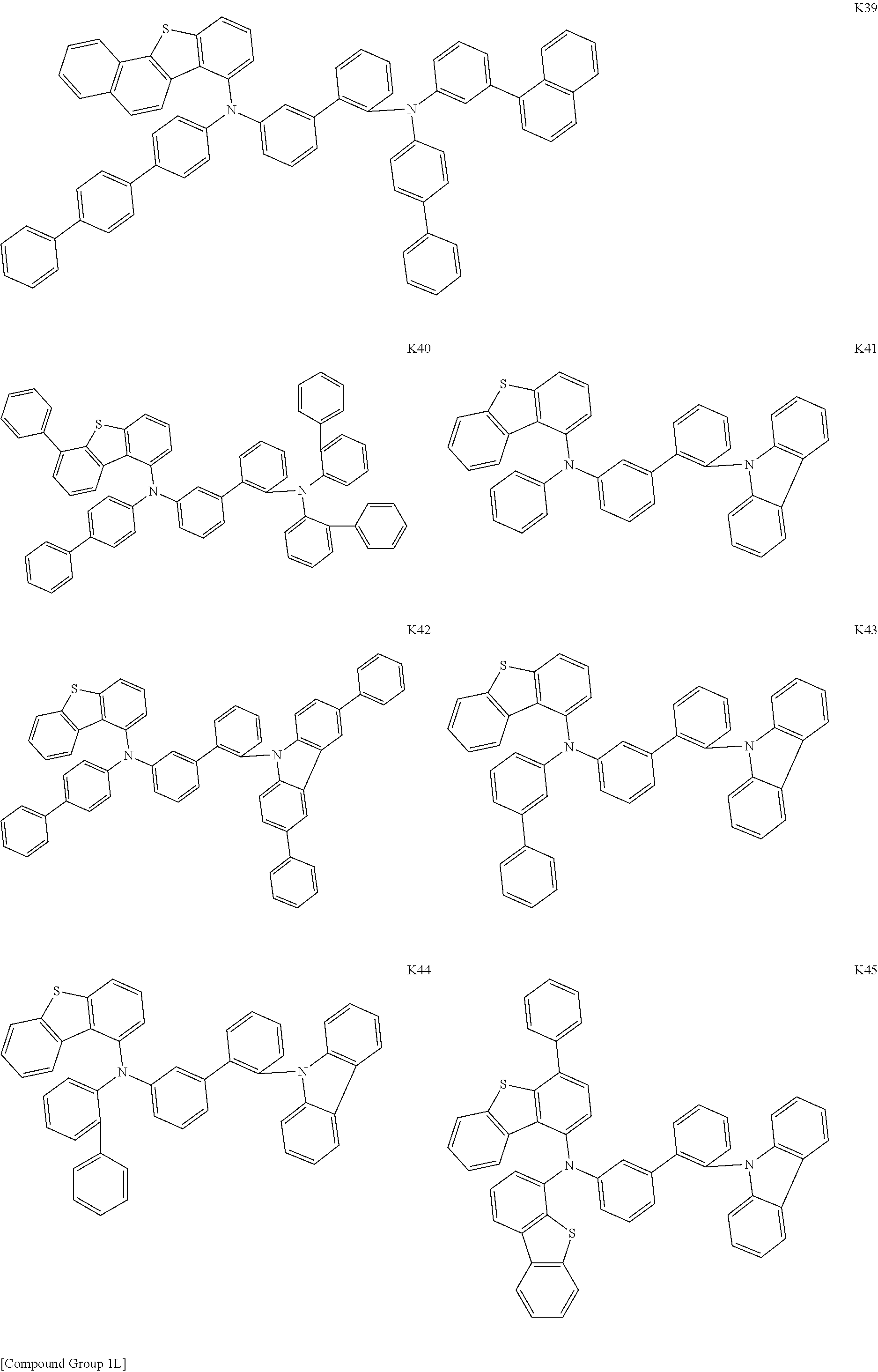 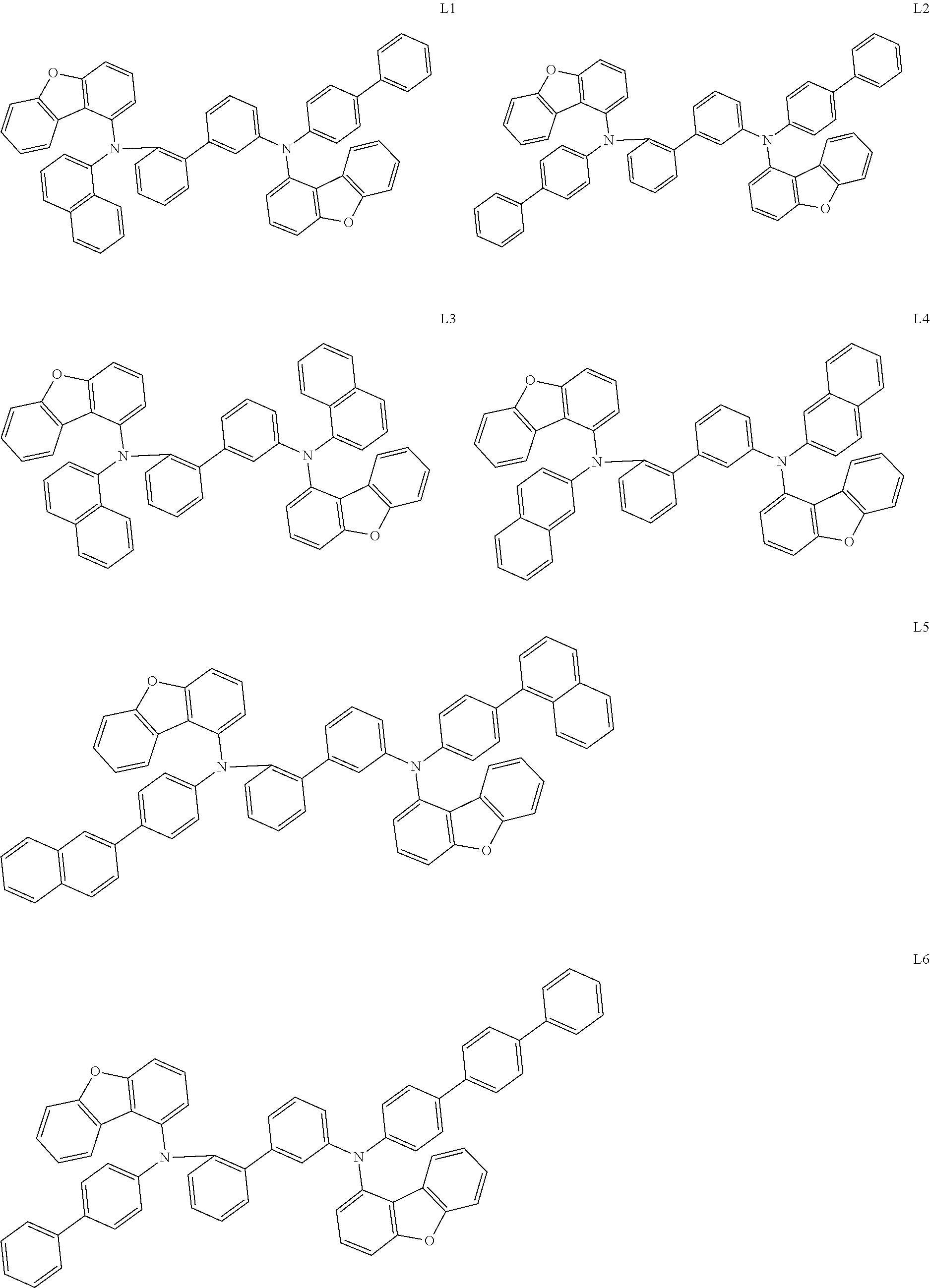 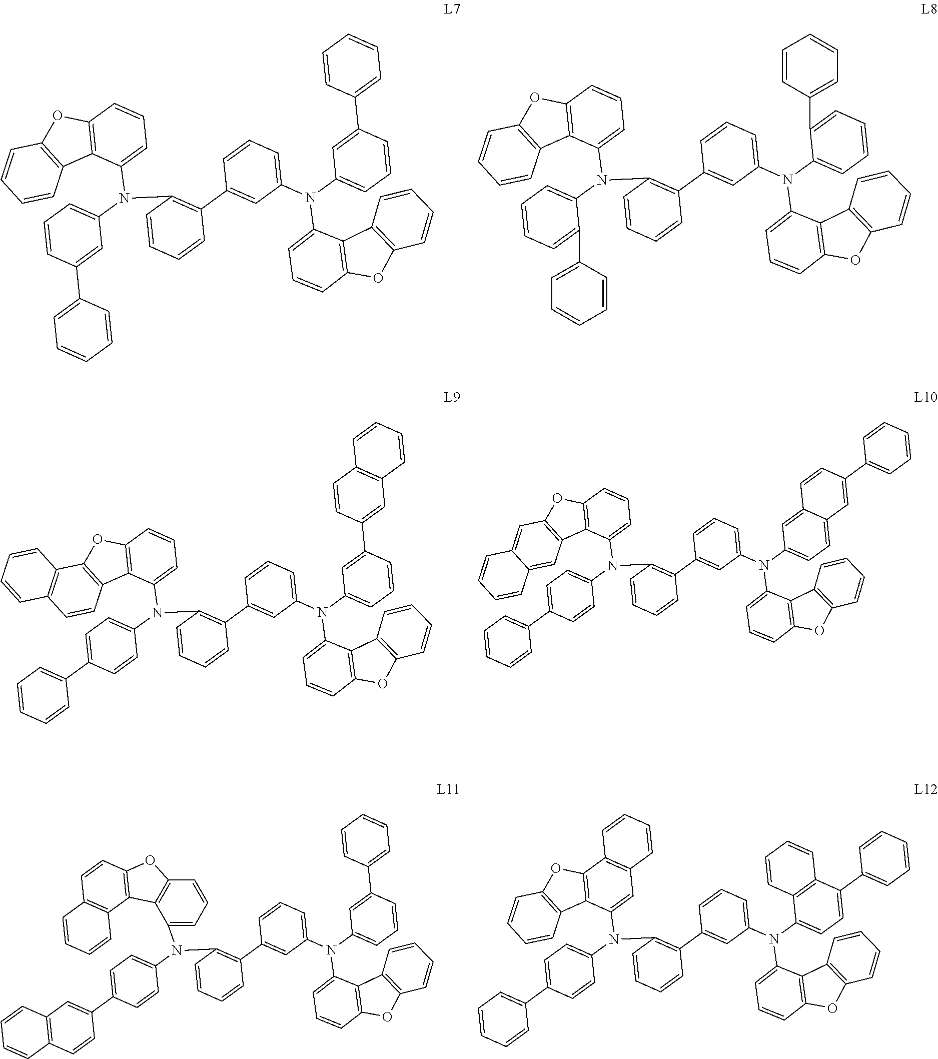 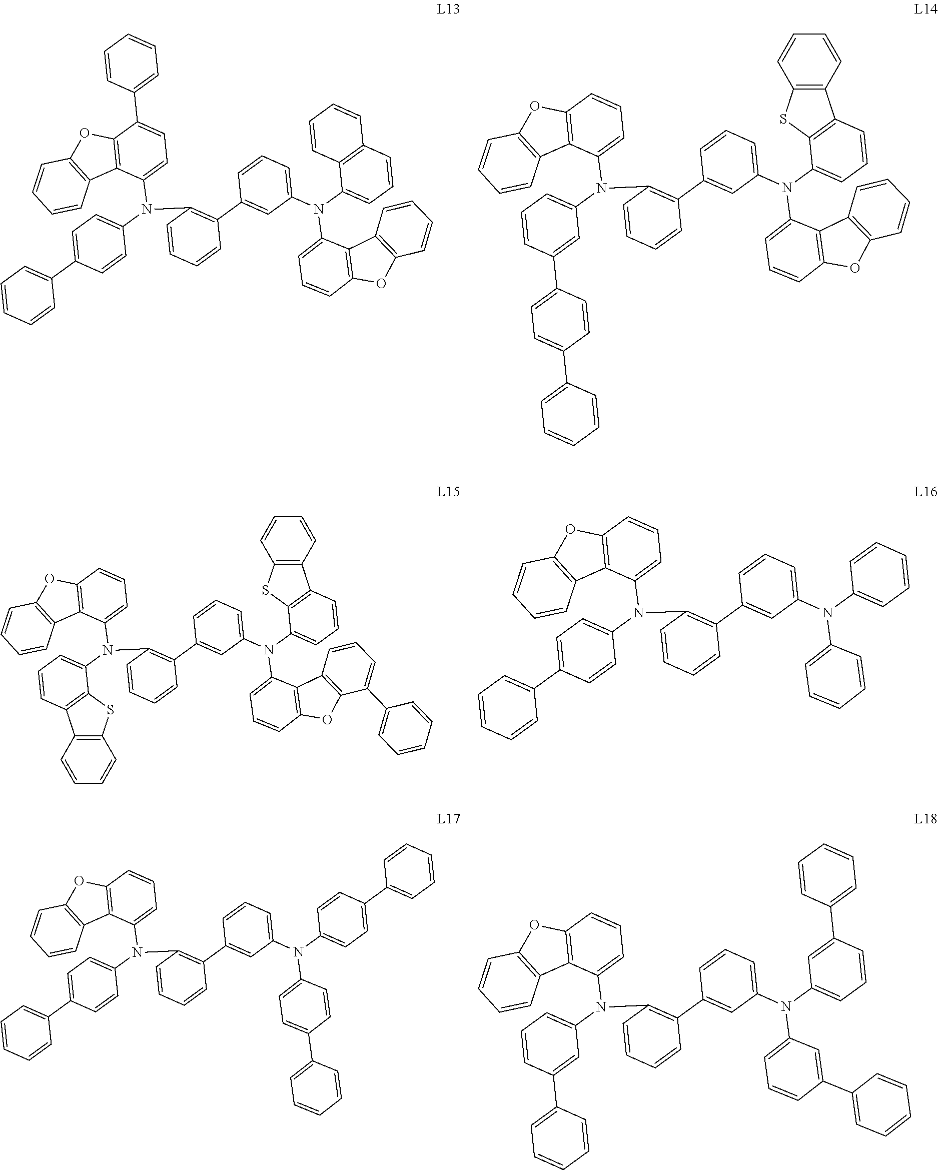 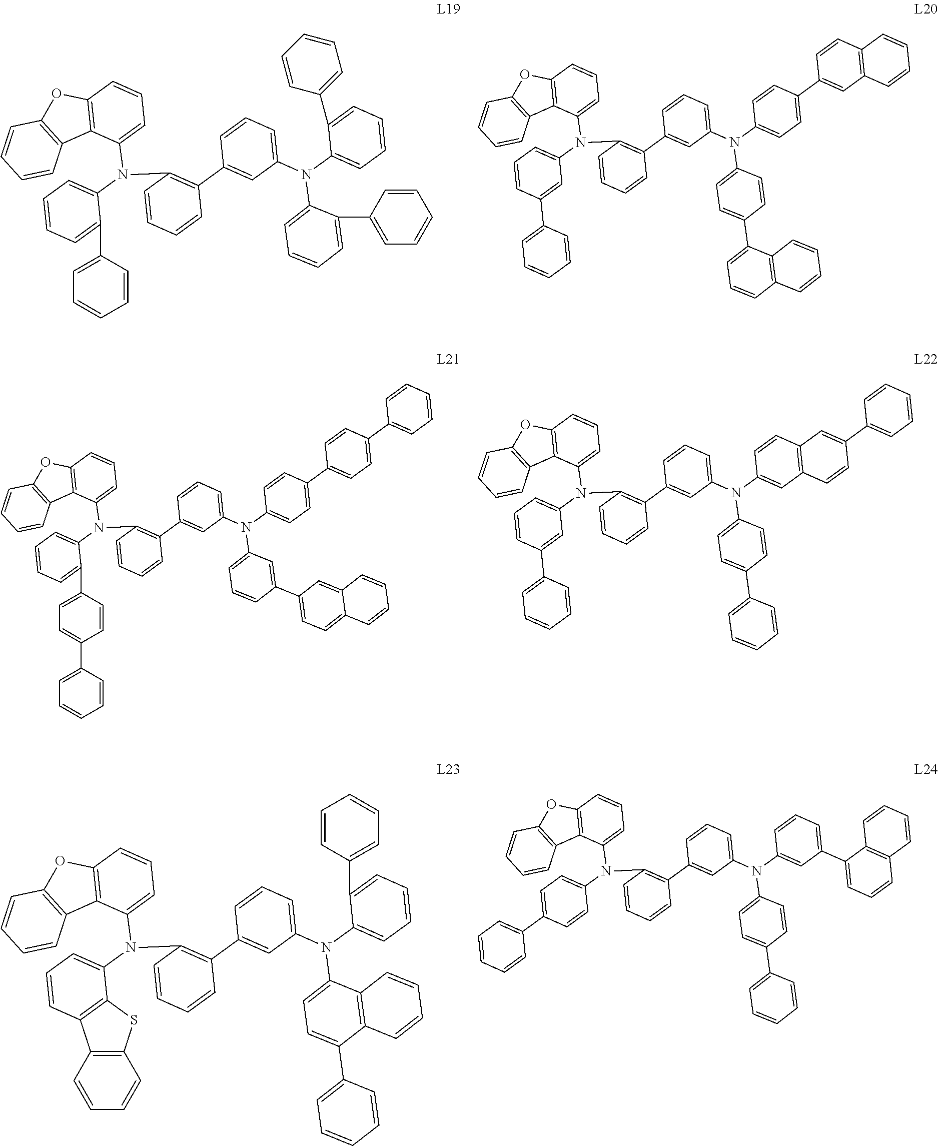 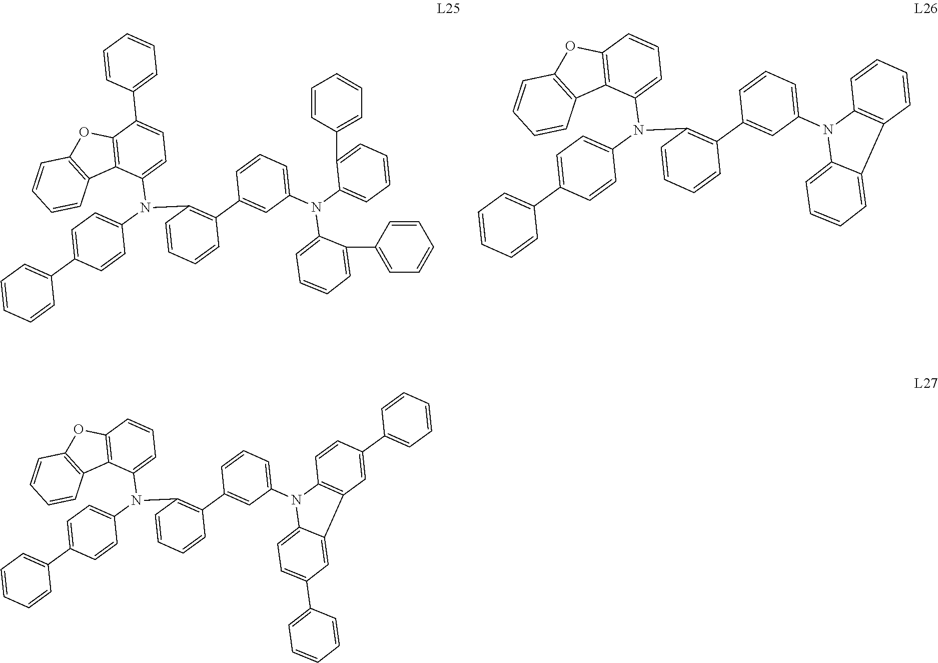 [SMILES: c1ccc(-c2ccc(N(c3ccccc3-c3cccc(N(c4ccccc4-c4ccccc4)c4ccccc4-c4ccccc4)c3)c3ccc(-c4ccccc4)c4oc5ccccc5c34)cc2)cc1 ; c1ccc(-c2ccc(N(c3ccccc3-c3cccc(-n4c5ccccc5c5ccccc54)c3)c3cccc4oc5ccccc5c34)cc2)cc1 ; c1ccc(-c2ccc(N(c3ccccc3-c3cccc(-n4c5ccc(-c6ccccc6)cc5c5cc(-c6ccccc6)ccc54)c3)c3cccc4oc5ccccc5c34)cc2)cc1] 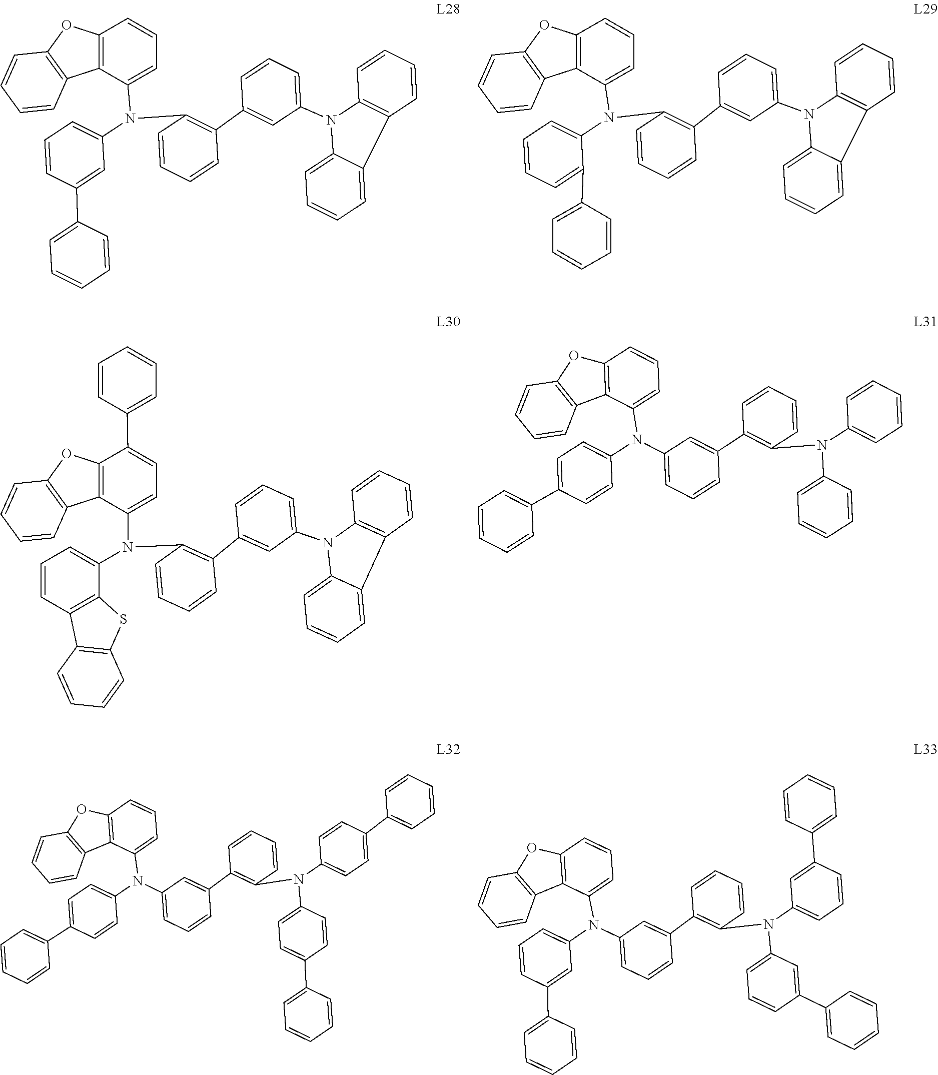 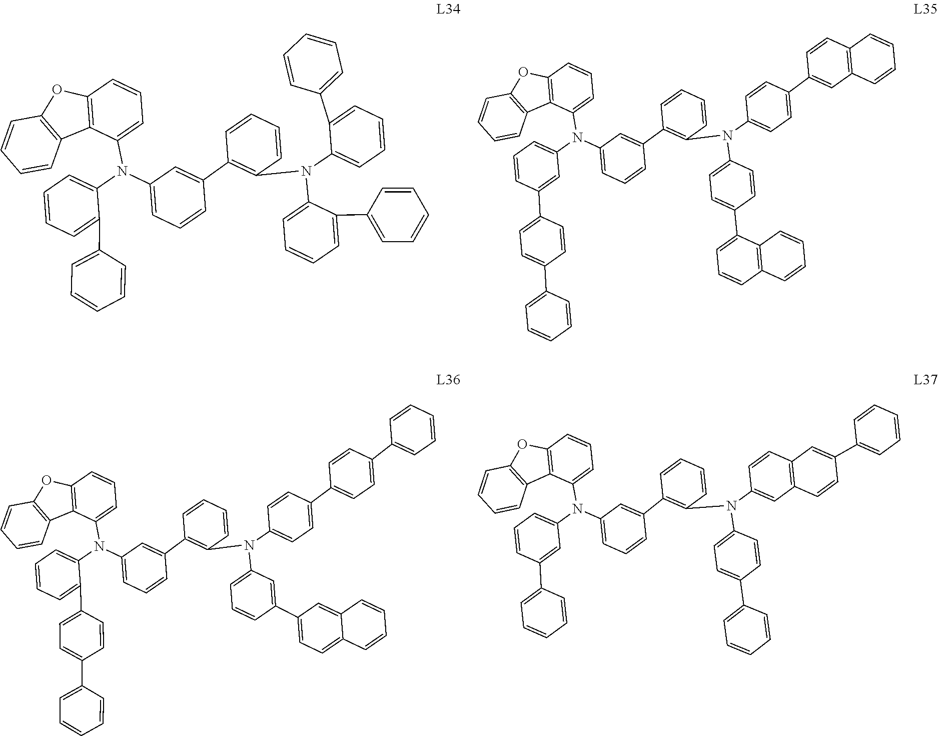 [SMILES: c1ccc(-c2ccccc2N(c2ccccc2-c2ccccc2)c2ccccc2-c2cccc(N(c3ccccc3-c3ccccc3)c3cccc4oc5ccccc5c34)c2)cc1 ; c1ccc(-c2ccc(-c3cccc(N(c4cccc(-c5ccccc5N(c5ccc(-c6ccc7ccccc7c6)cc5)c5ccc(-c6cccc7ccccc67)cc5)c4)c4cccc5oc6ccccc6c45)c3)cc2)cc1 ; c1ccc(-c2ccc(-c3ccc(N(c4cccc(-c5ccc6ccccc6c5)c4)c4ccccc4-c4cccc(N(c5ccccc5-c5ccc(-c6ccccc6)cc5)c5cccc6oc7ccccc7c56)c4)cc3)cc2)cc1 ; c1ccc(-c2ccc(N(c3ccc4cc(-c5ccccc5)ccc4c3)c3ccccc3-c3cccc(N(c4cccc(-c5ccccc5)c4)c4cccc5oc6ccccc6c45)c3)cc2)cc1] 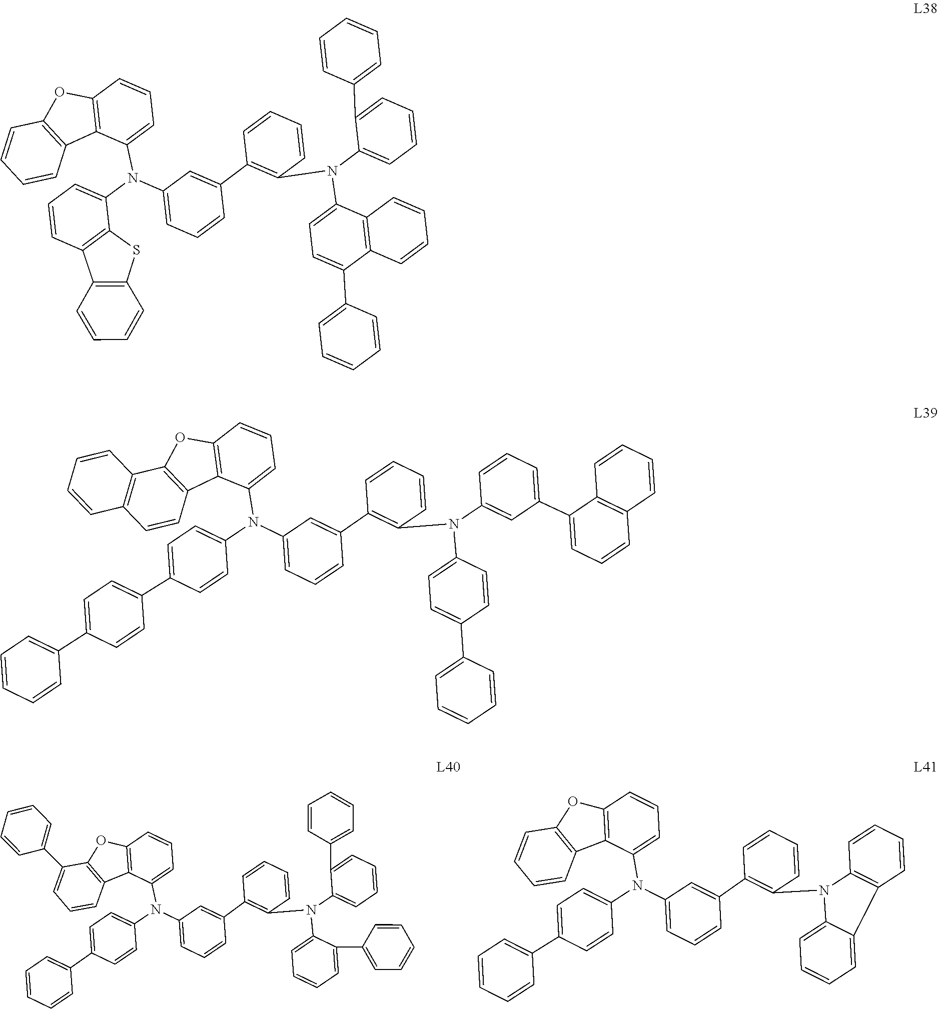 [SMILES: c1ccc(-c2ccccc2N(c2ccccc2-c2cccc(N(c3cccc4c3sc3ccccc34)c3cccc4oc5ccccc5c34)c2)c2ccc(-c3ccccc3)c3ccccc23)cc1 ; c1ccc(-c2ccc(-c3ccc(N(c4cccc(-c5ccccc5N(c5ccc(-c6ccccc6)cc5)c5cccc(-c6cccc7ccccc67)c5)c4)c4cccc5oc6c7ccccc7ccc6c45)cc3)cc2)cc1 ; c1ccc(-c2ccc(N(c3cccc(-c4ccccc4N(c4ccccc4-c4ccccc4)c4ccccc4-c4ccccc4)c3)c3cccc4oc5c(-c6ccccc6)cccc5c34)cc2)cc1 ; c1ccc(-c2ccc(N(c3cccc(-c4ccccc4-n4c5ccccc5c5ccccc54)c3)c3cccc4oc5ccccc5c34)cc2)cc1] 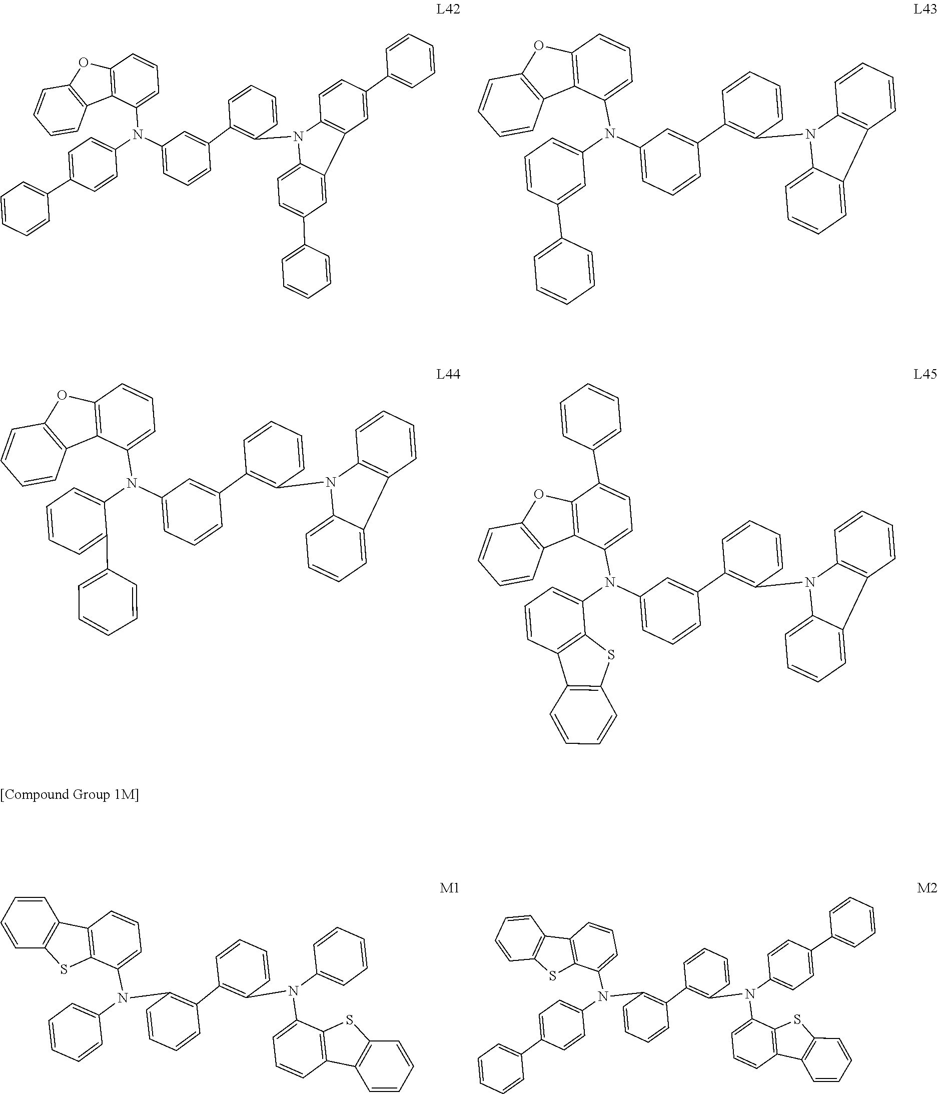 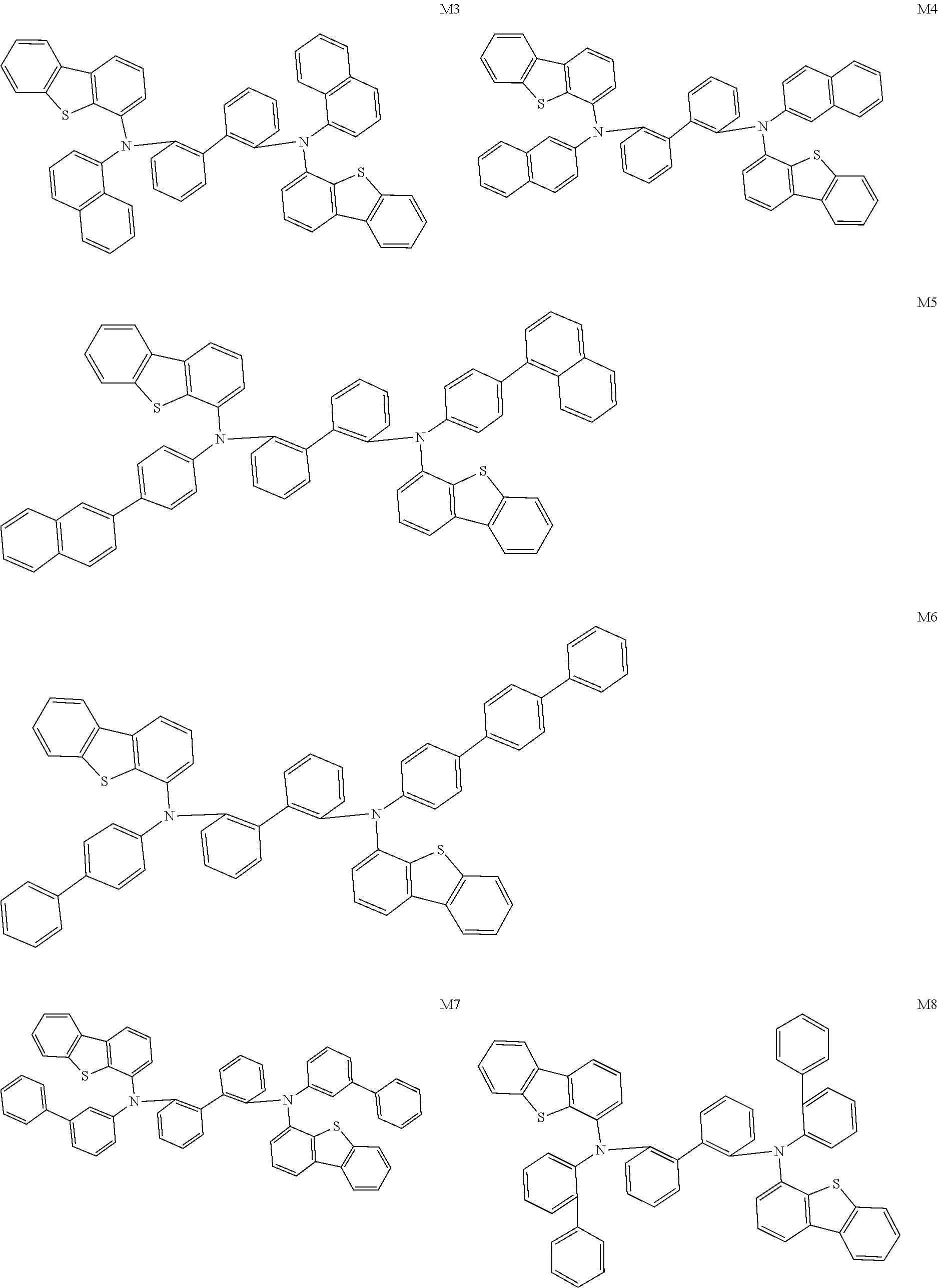 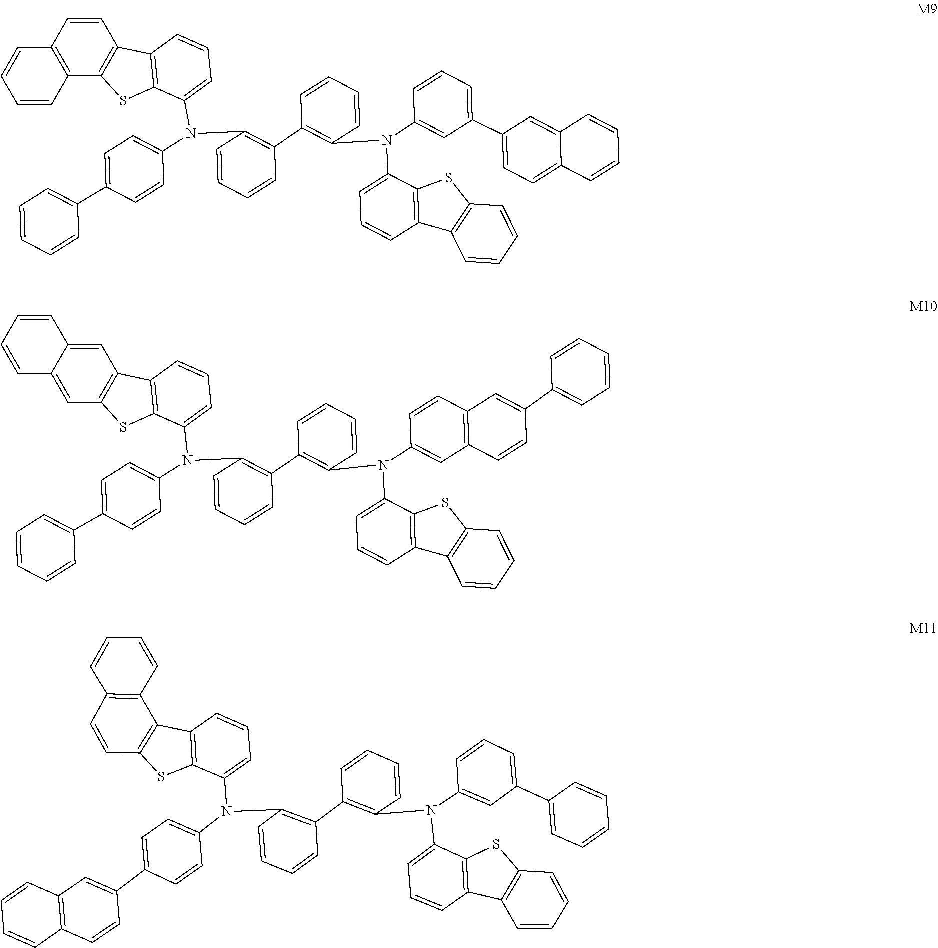 [SMILES: c1ccc(-c2ccc(N(c3ccccc3-c3ccccc3N(c3cccc(-c4ccc5ccccc5c4)c3)c3cccc4c3sc3ccccc34)c3cccc4c3sc3c5ccccc5ccc43)cc2)cc1 ; c1ccc(-c2ccc(N(c3ccccc3-c3ccccc3N(c3ccc4cc(-c5ccccc5)ccc4c3)c3cccc4c3sc3ccccc34)c3cccc4c3sc3cc5ccccc5cc34)cc2)cc1 ; c1ccc(-c2cccc(N(c3ccccc3-c3ccccc3N(c3ccc(-c4ccc5ccccc5c4)cc3)c3cccc4c3sc3ccc5ccccc5c34)c3cccc4c3sc3ccccc34)c2)cc1] 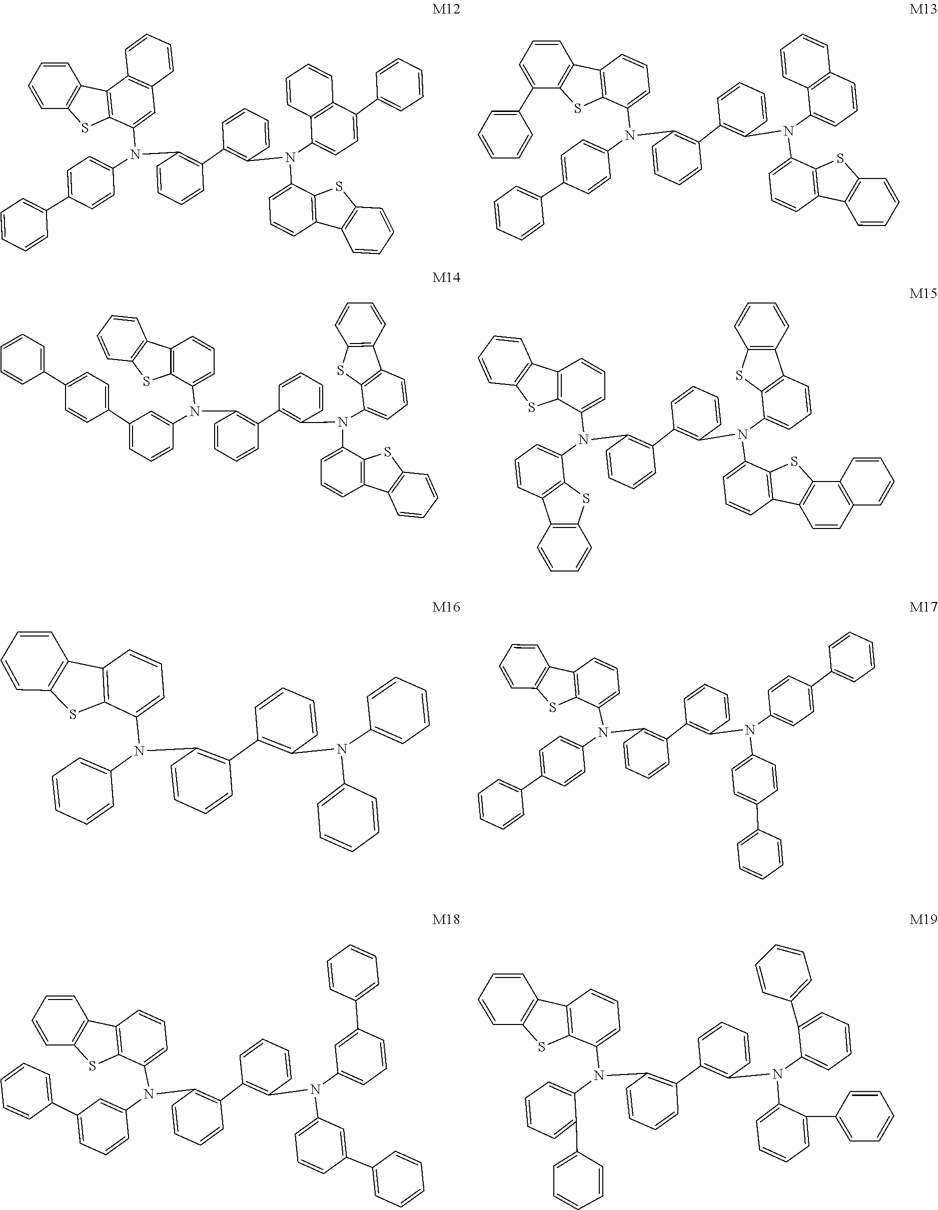 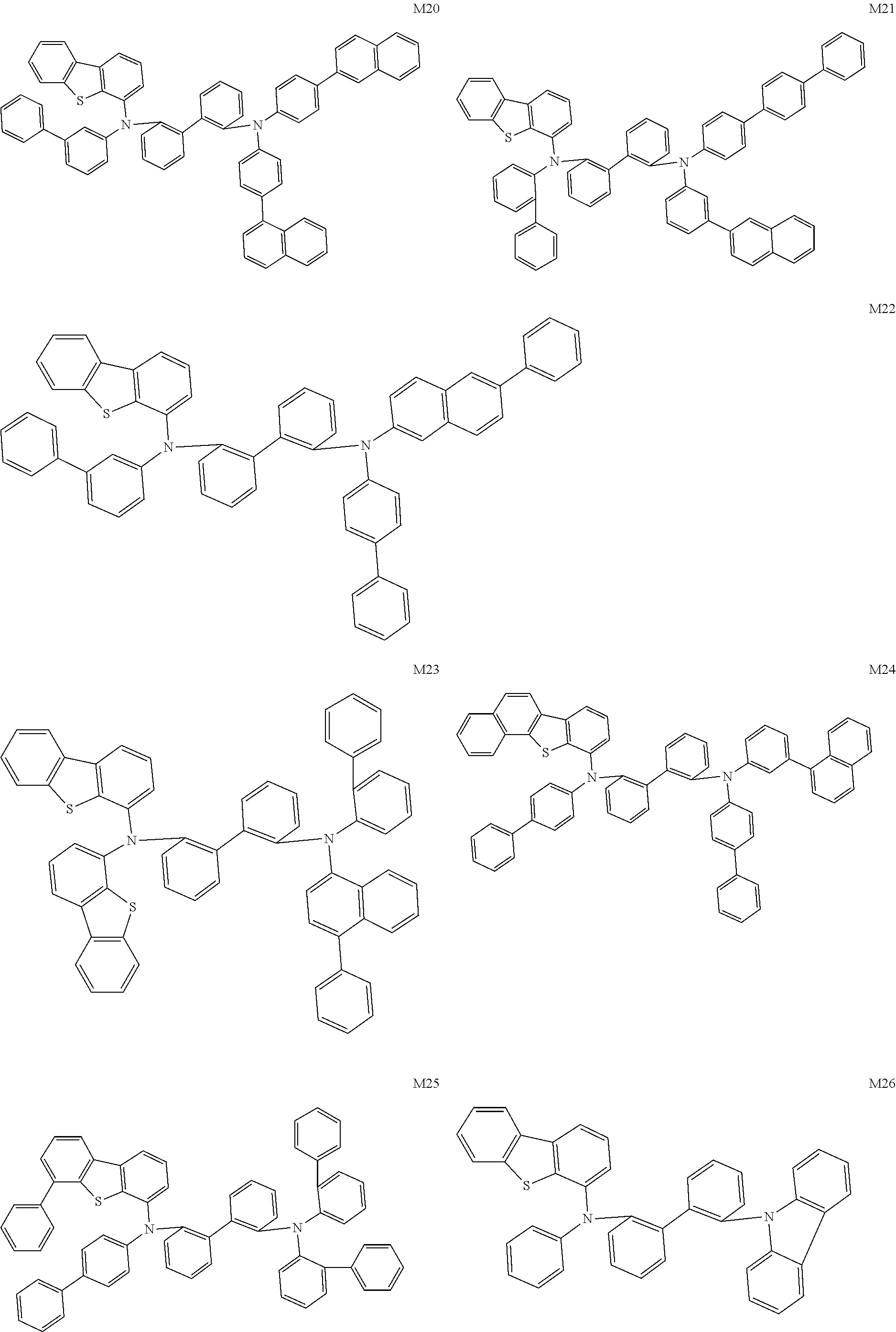 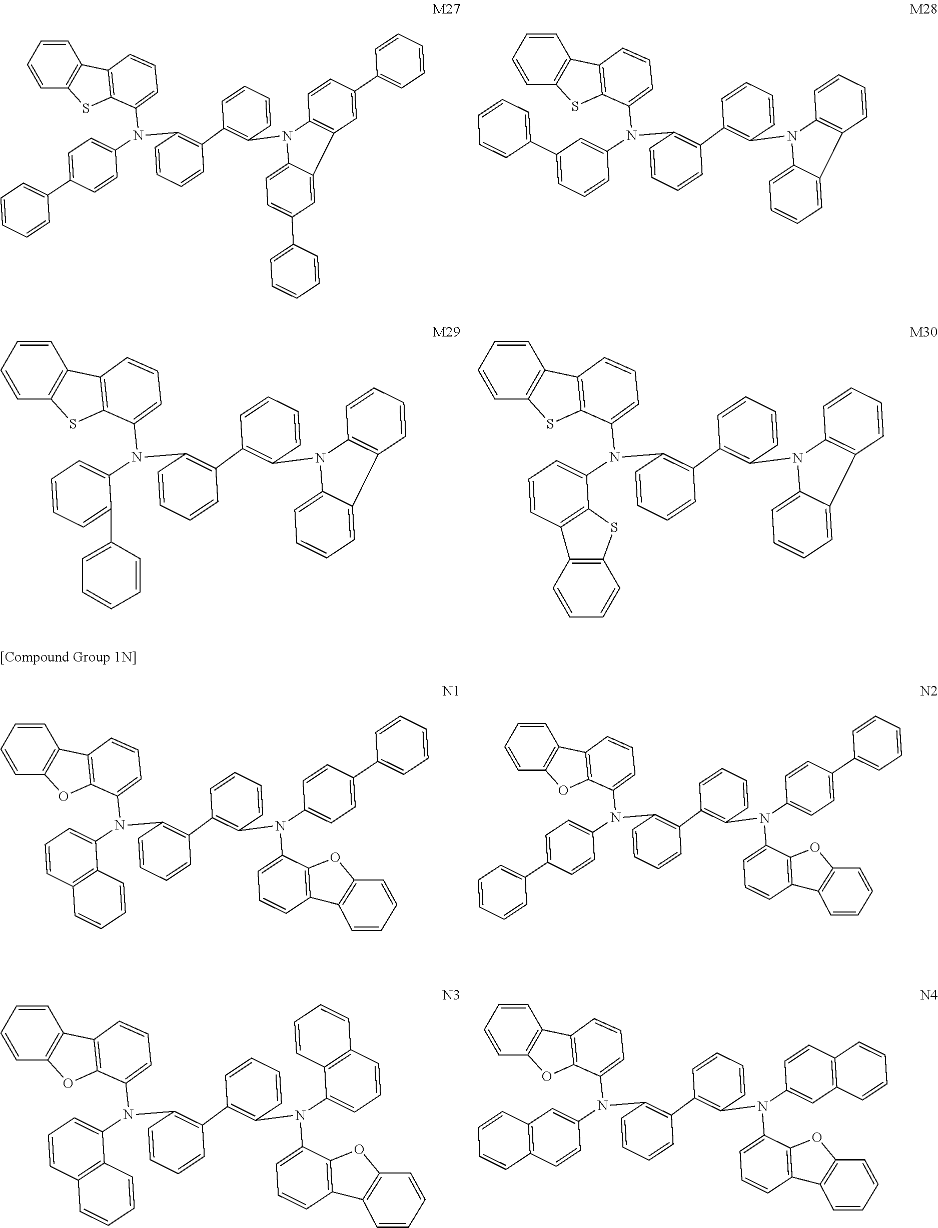 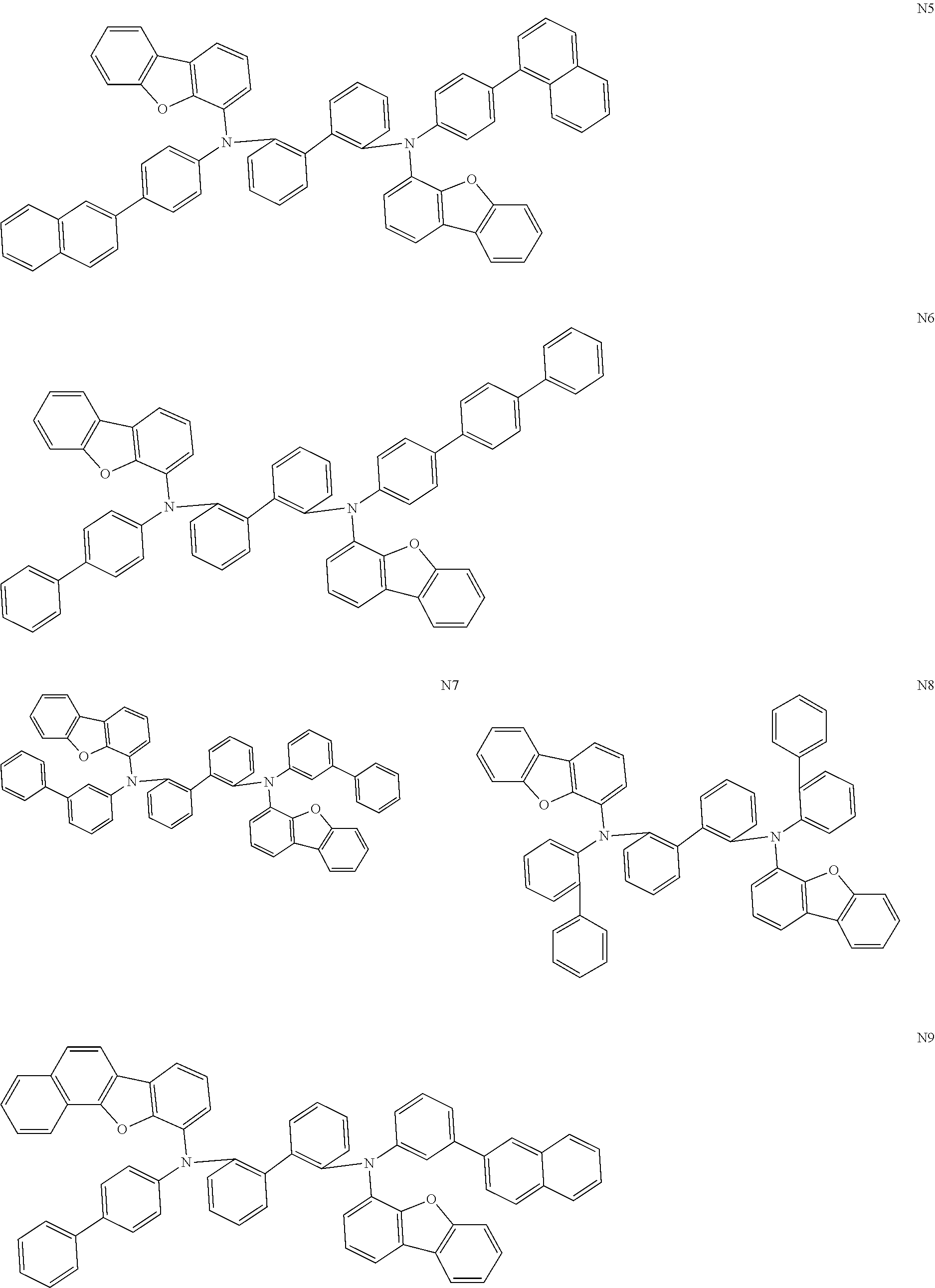 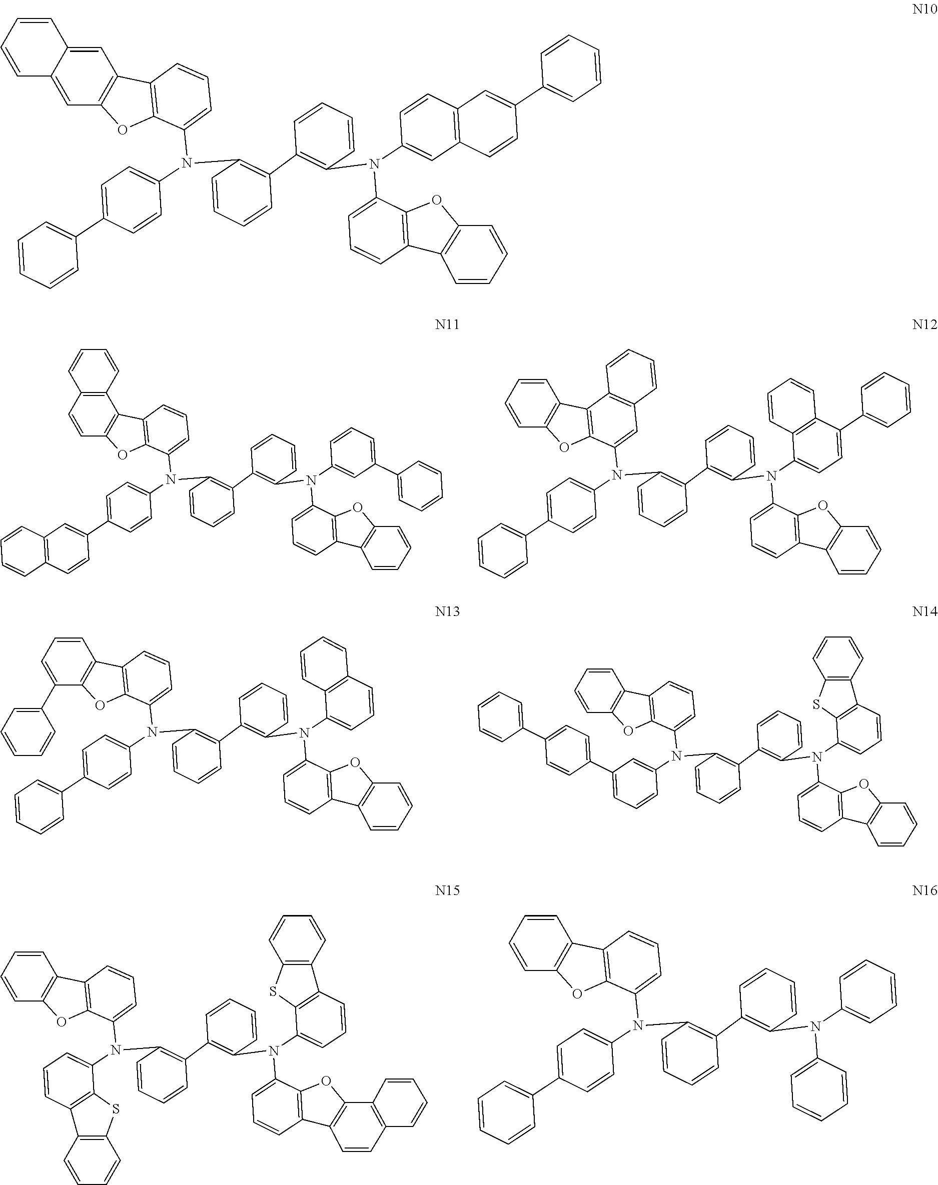 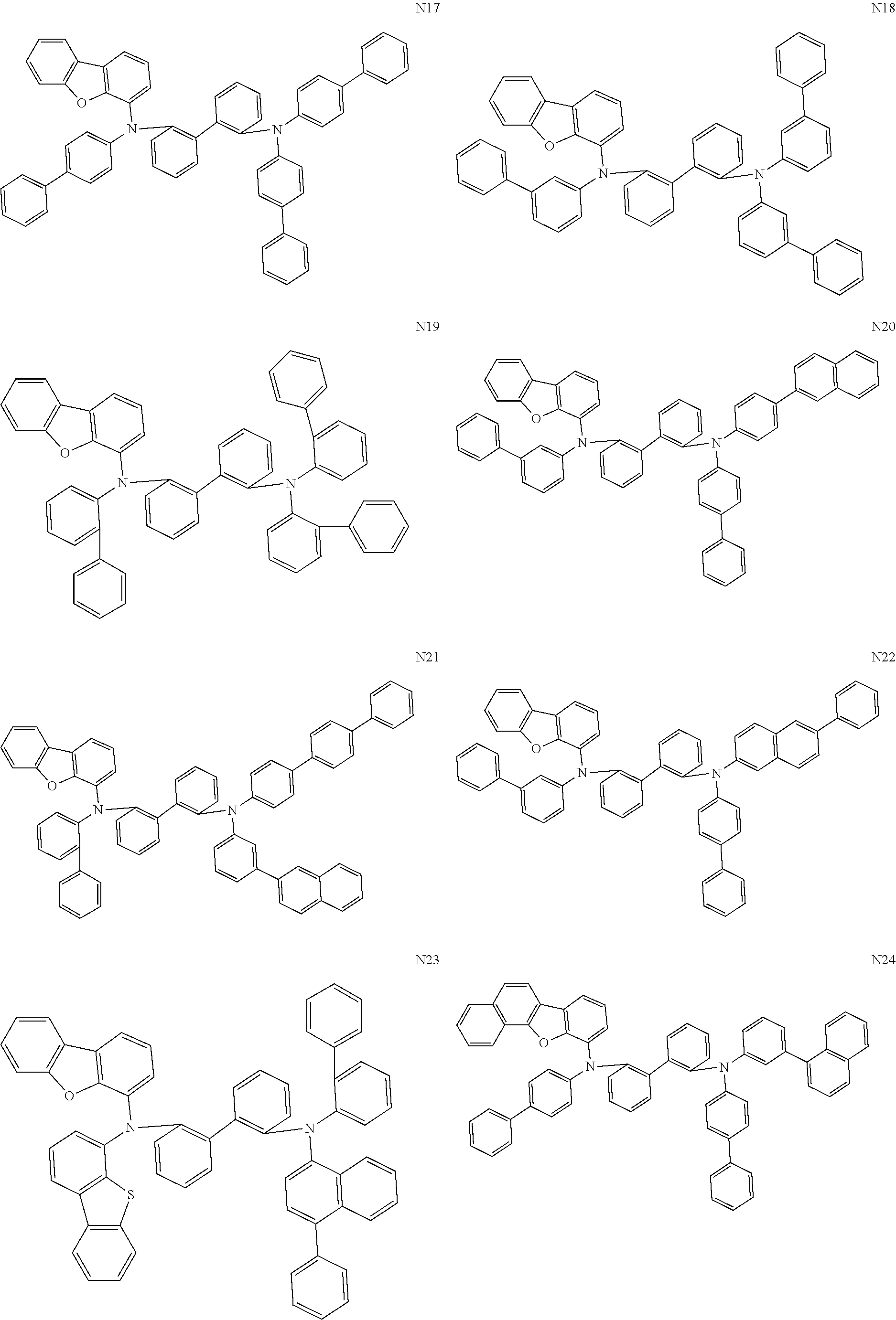 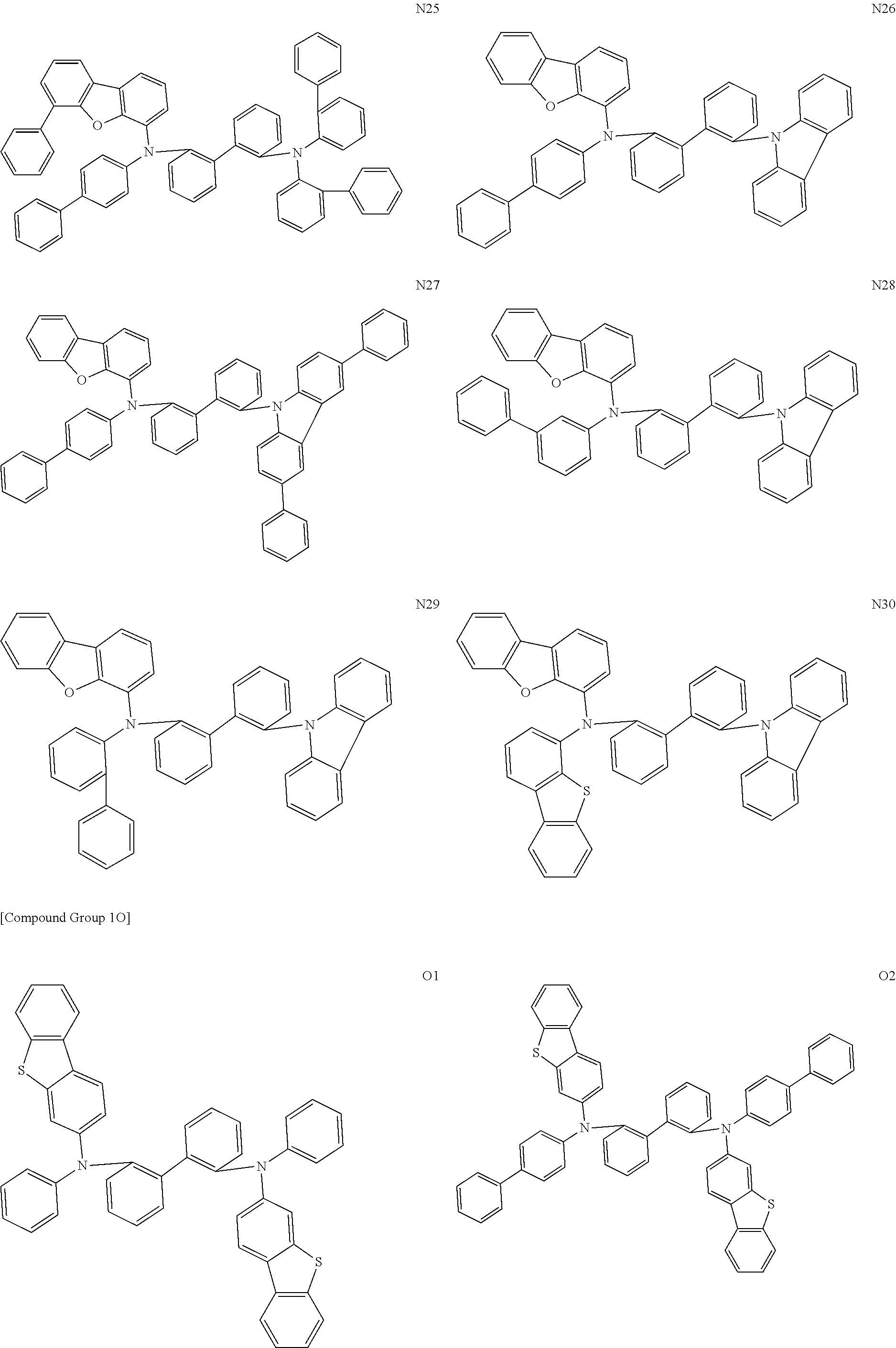 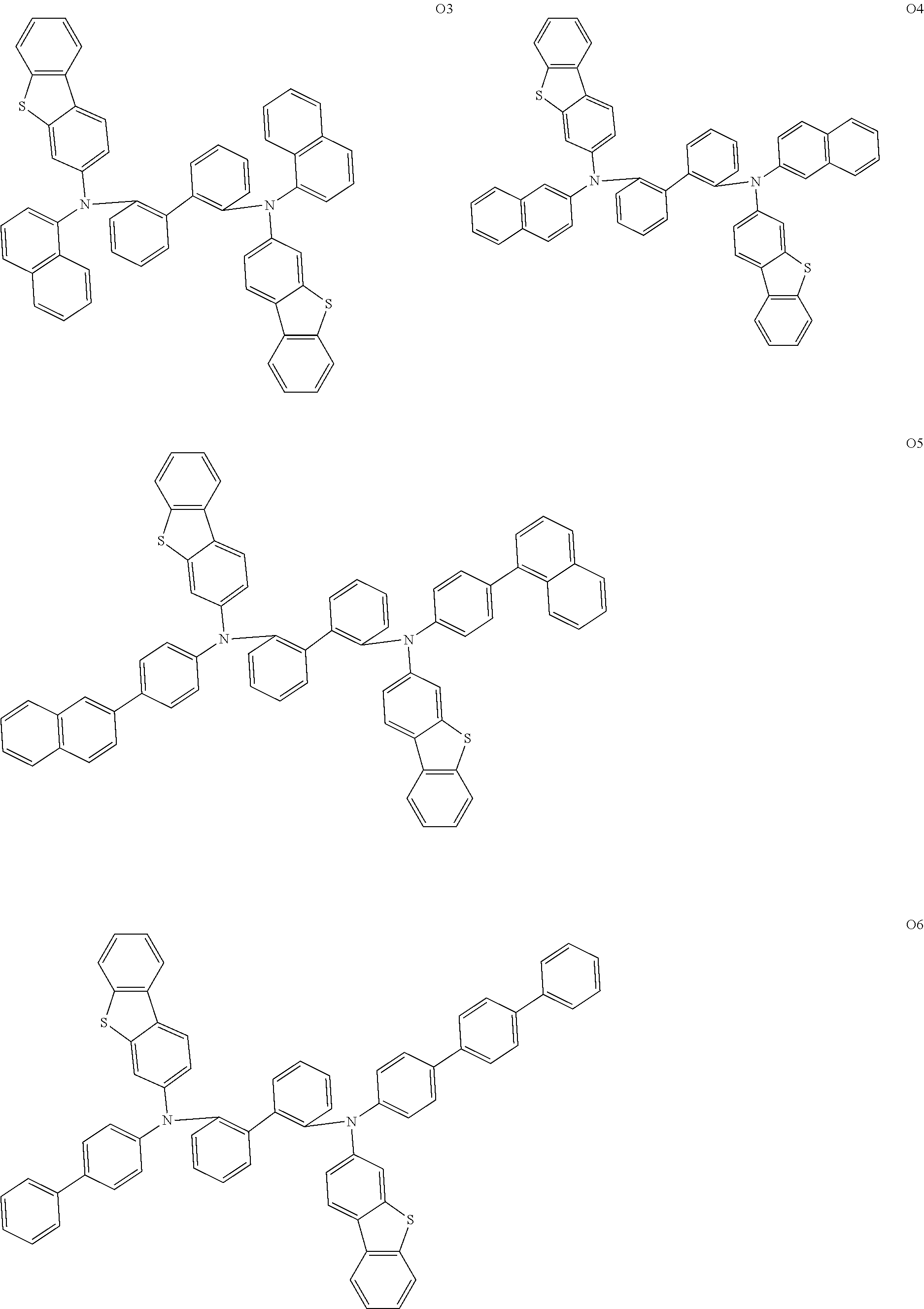 [SMILES: c1ccc(N(c2ccc3c(c2)sc2ccccc23)c2cccc3ccccc23)c(-c2ccccc2N(c2ccc3c(c2)sc2ccccc23)c2cccc3ccccc23)c1 ; c1ccc(N(c2ccc3ccccc3c2)c2ccc3c(c2)sc2ccccc23)c(-c2ccccc2N(c2ccc3ccccc3c2)c2ccc3c(c2)sc2ccccc23)c1 ; c1ccc(N(c2ccc(-c3ccc4ccccc4c3)cc2)c2ccc3c(c2)sc2ccccc23)c(-c2ccccc2N(c2ccc(-c3cccc4ccccc34)cc2)c2ccc3c(c2)sc2ccccc23)c1 ; c1ccc(-c2ccc(-c3ccc(N(c4ccc5c(c4)sc4ccccc45)c4ccccc4-c4ccccc4N(c4ccc(-c5ccccc5)cc4)c4ccc5c(c4)sc4ccccc45)cc3)cc2)cc1] 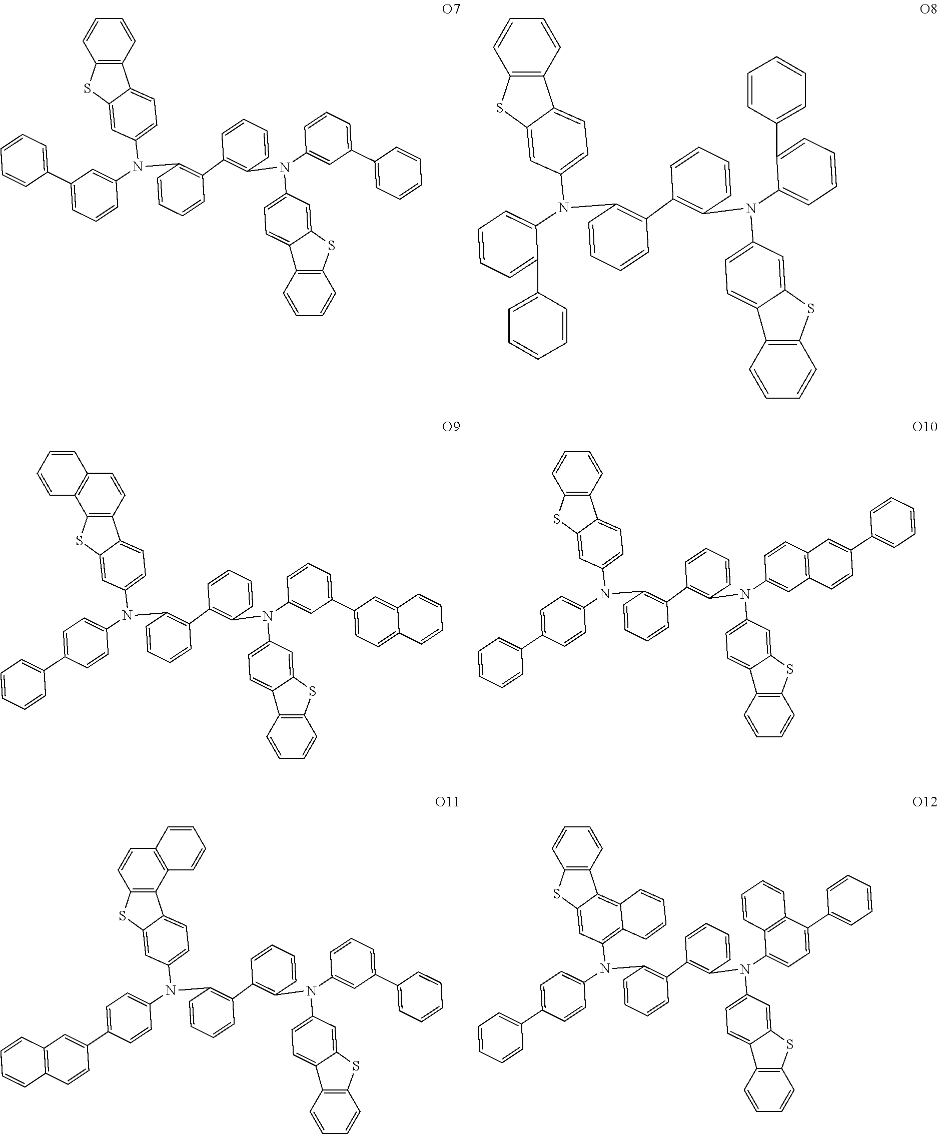 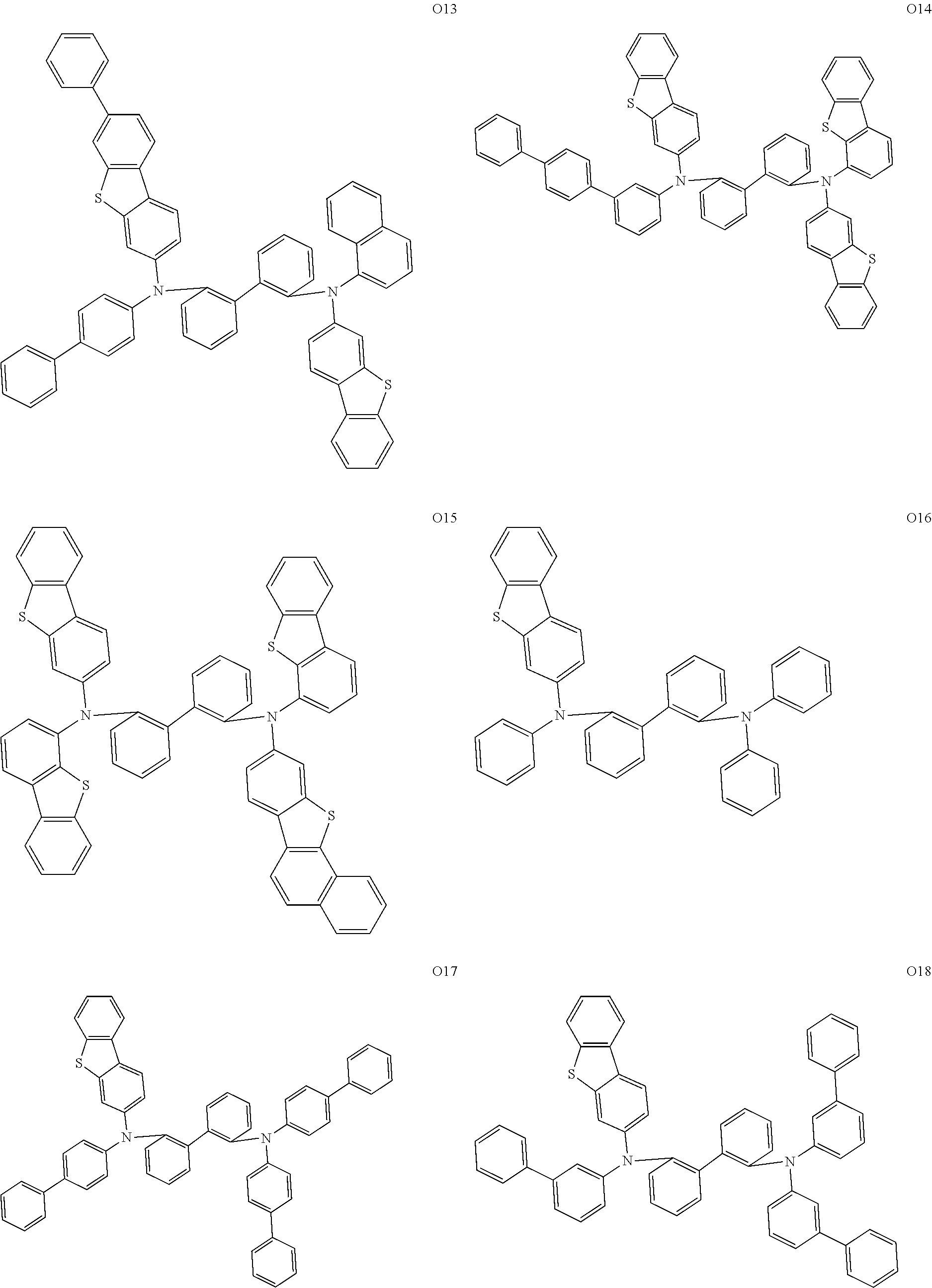 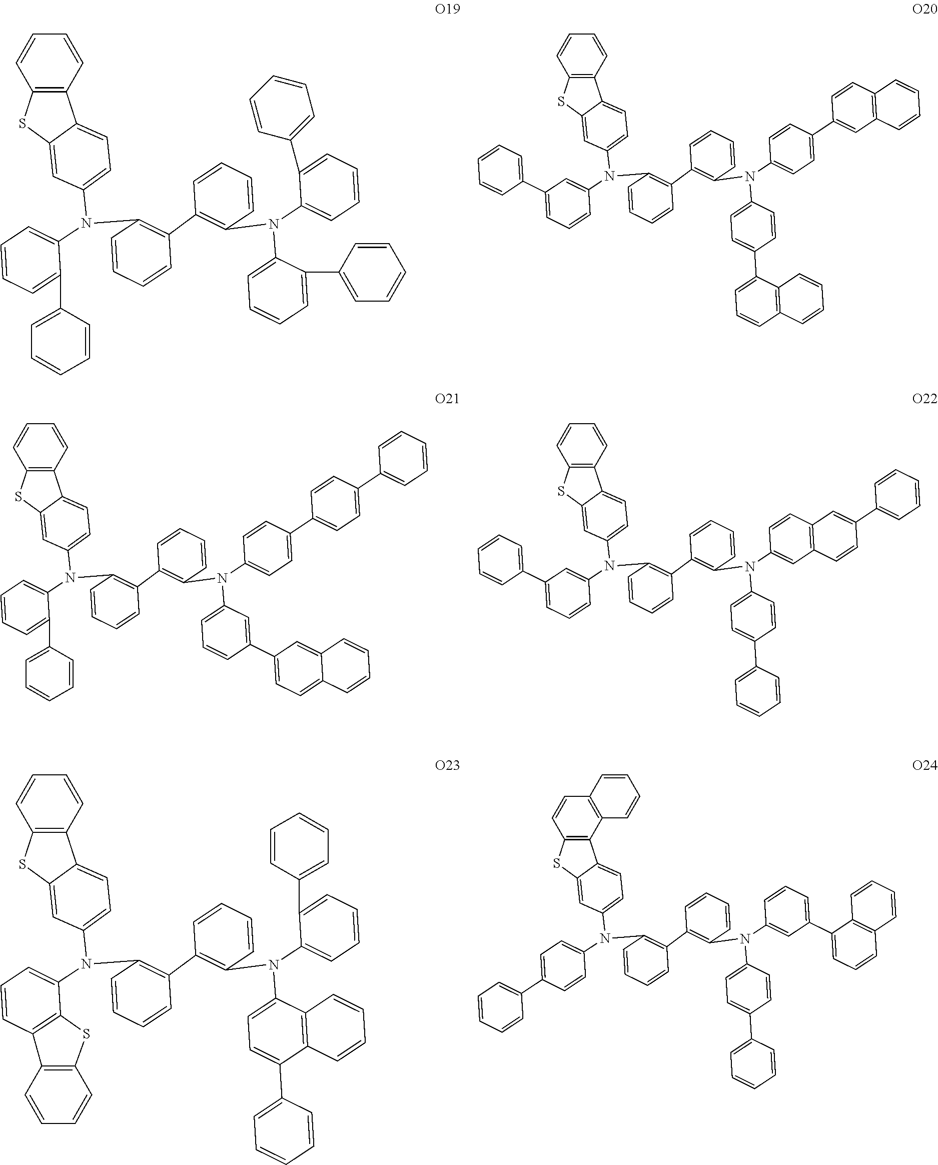 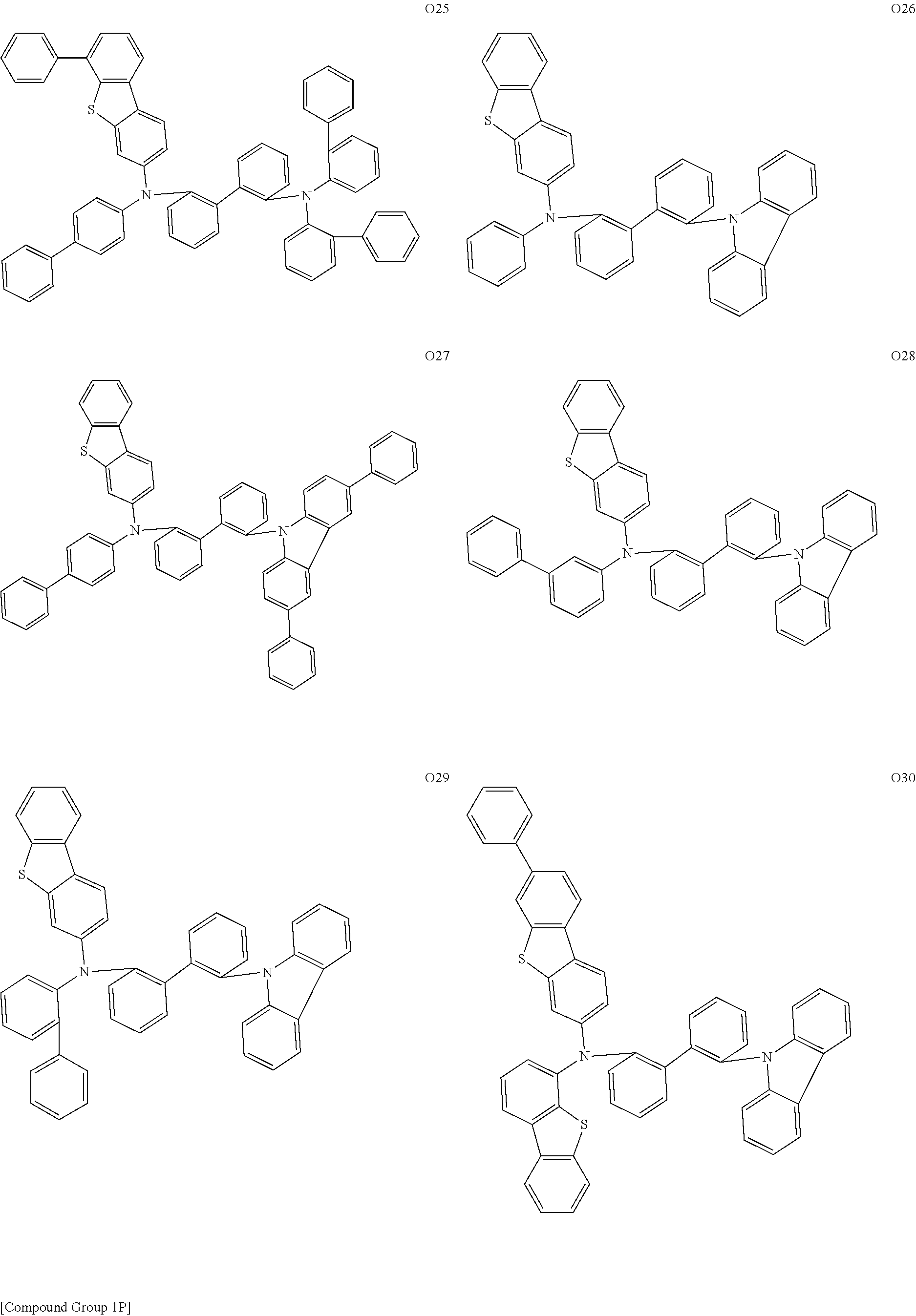 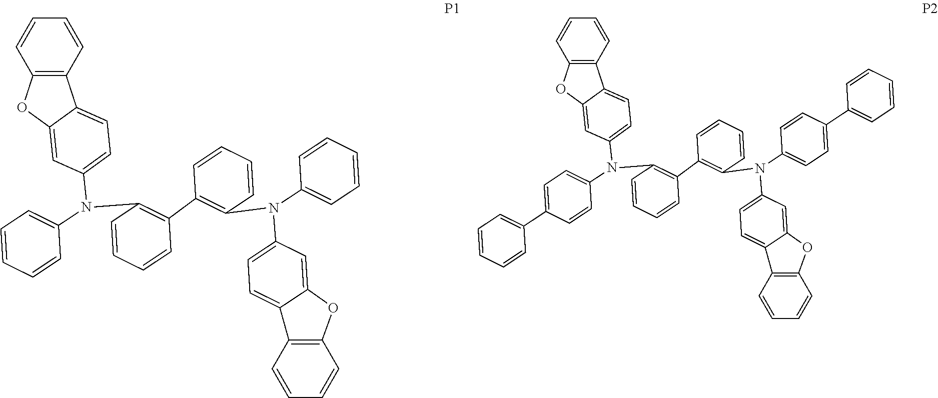 [SMILES: c1ccc(N(c2ccc3c(c2)oc2ccccc23)c2ccccc2-c2ccccc2N(c2ccccc2)c2ccc3c(c2)oc2ccccc23)cc1 ; c1ccc(-c2ccc(N(c3ccc4c(c3)oc3ccccc34)c3ccccc3-c3ccccc3N(c3ccc(-c4ccccc4)cc3)c3ccc4c(c3)oc3ccccc34)cc2)cc1] 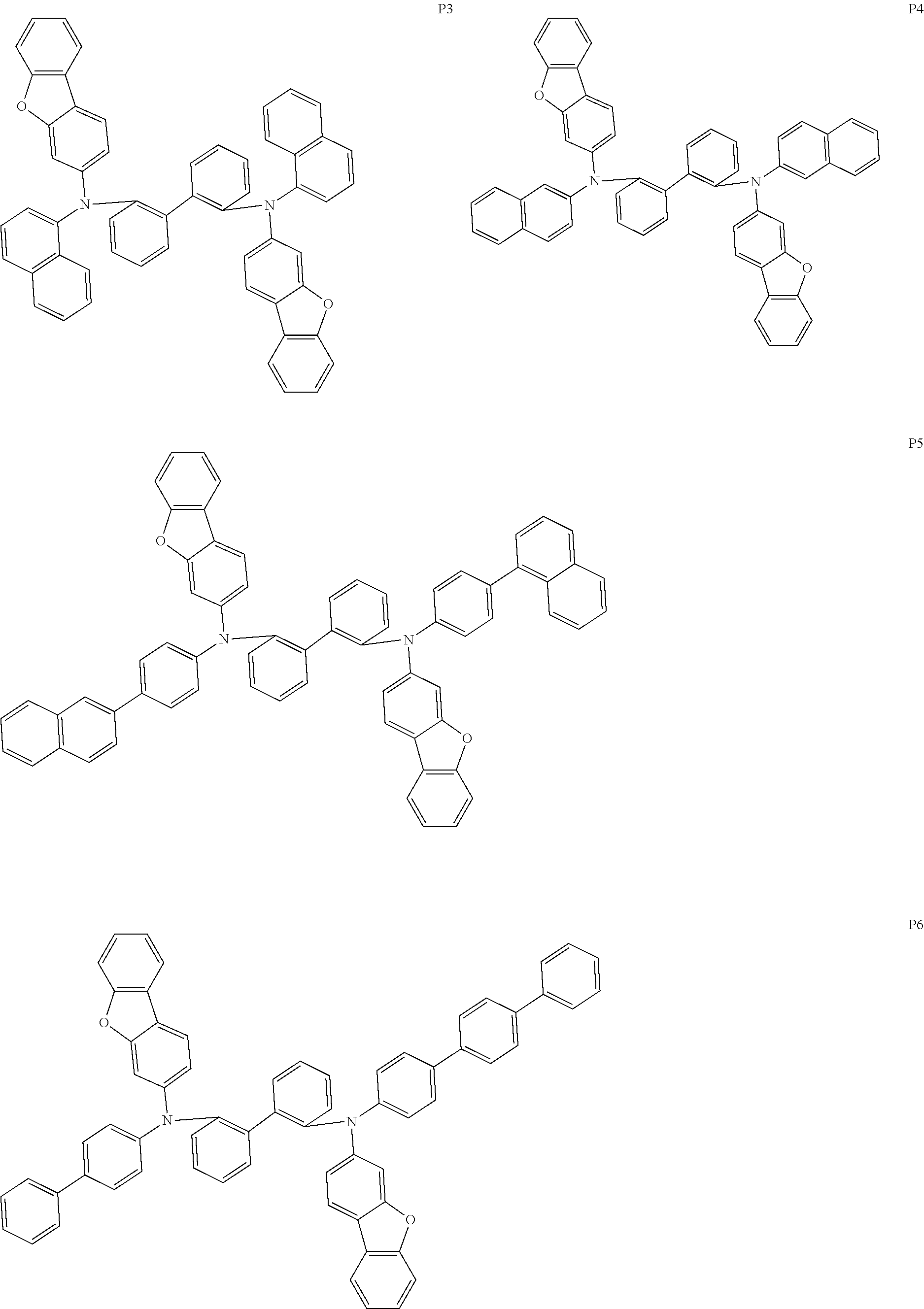 [SMILES: c1ccc(N(c2ccc3c(c2)oc2ccccc23)c2cccc3ccccc23)c(-c2ccccc2N(c2ccc3c(c2)oc2ccccc23)c2cccc3ccccc23)c1 ; c1ccc(N(c2ccc3ccccc3c2)c2ccc3c(c2)oc2ccccc23)c(-c2ccccc2N(c2ccc3ccccc3c2)c2ccc3c(c2)oc2ccccc23)c1 ; c1ccc(N(c2ccc(-c3ccc4ccccc4c3)cc2)c2ccc3c(c2)oc2ccccc23)c(-c2ccccc2N(c2ccc(-c3cccc4ccccc34)cc2)c2ccc3c(c2)oc2ccccc23)c1 ; c1ccc(-c2ccc(-c3ccc(N(c4ccc5c(c4)oc4ccccc45)c4ccccc4-c4ccccc4N(c4ccc(-c5ccccc5)cc4)c4ccc5c(c4)oc4ccccc45)cc3)cc2)cc1] 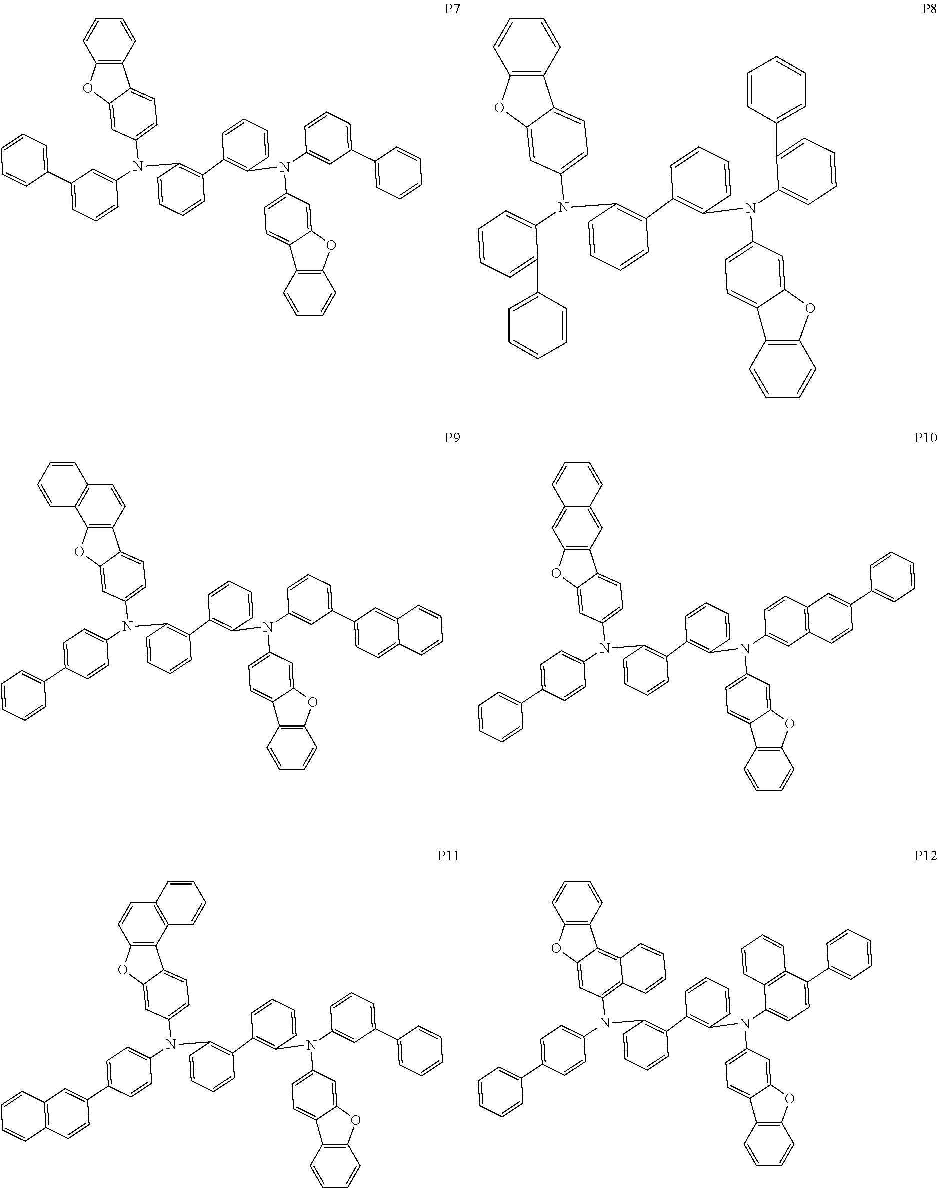 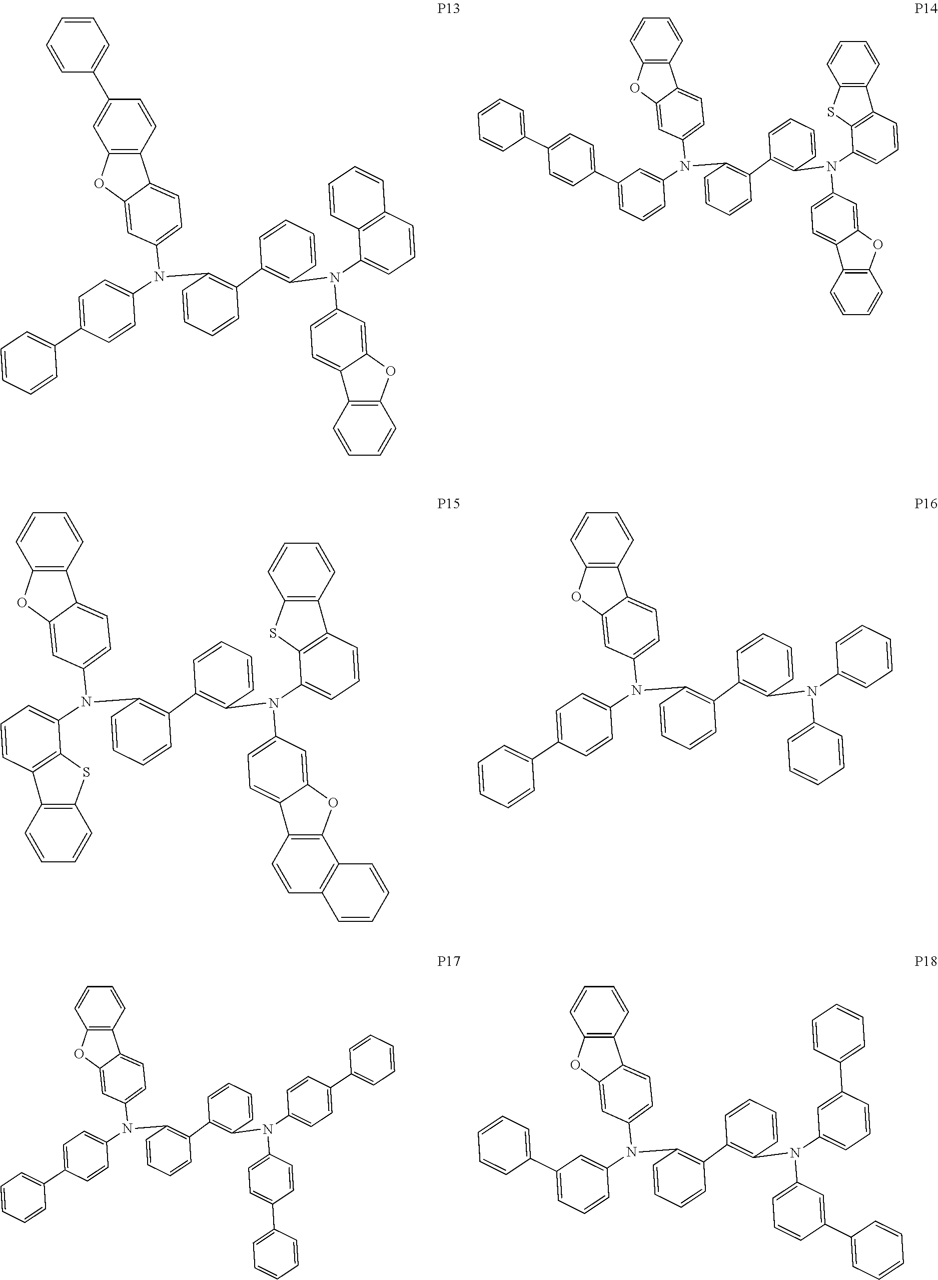 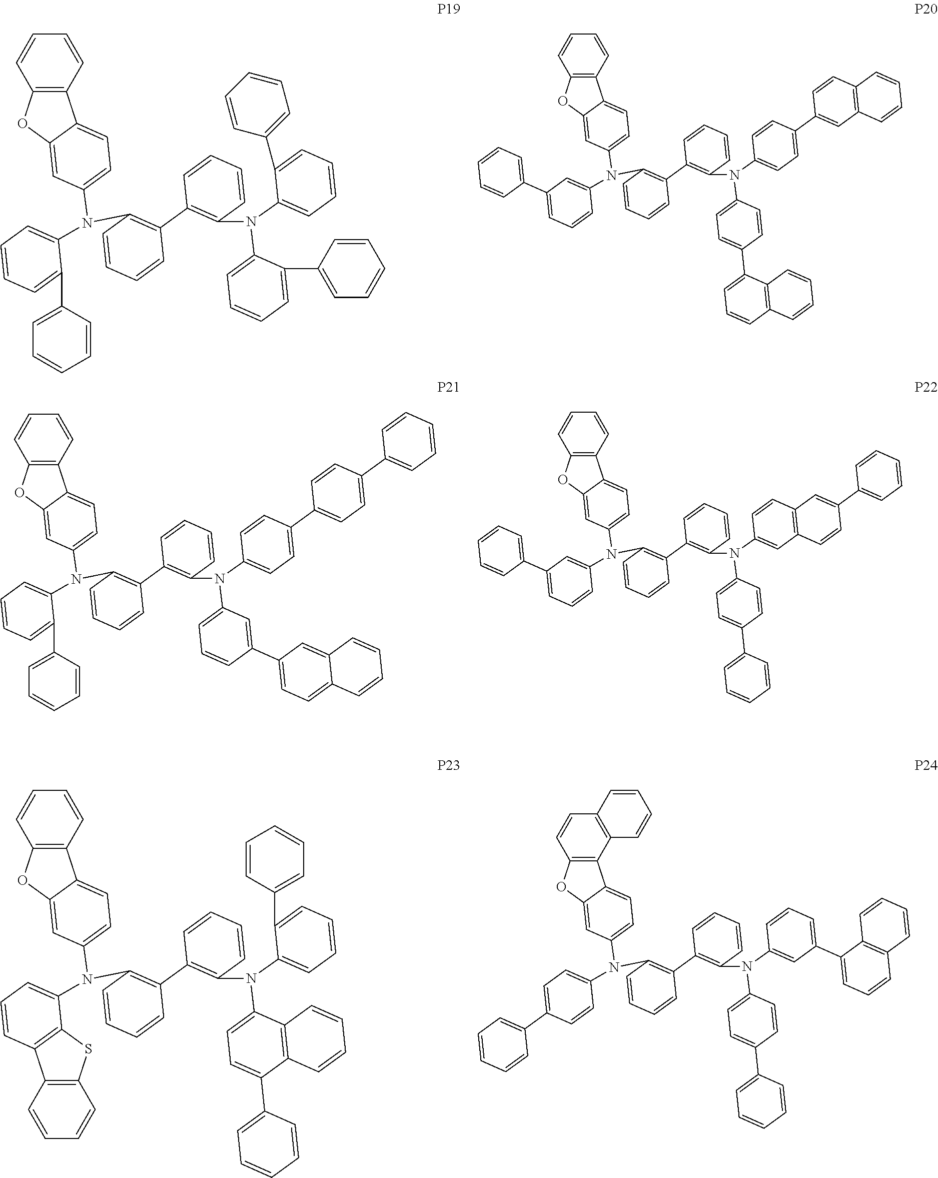 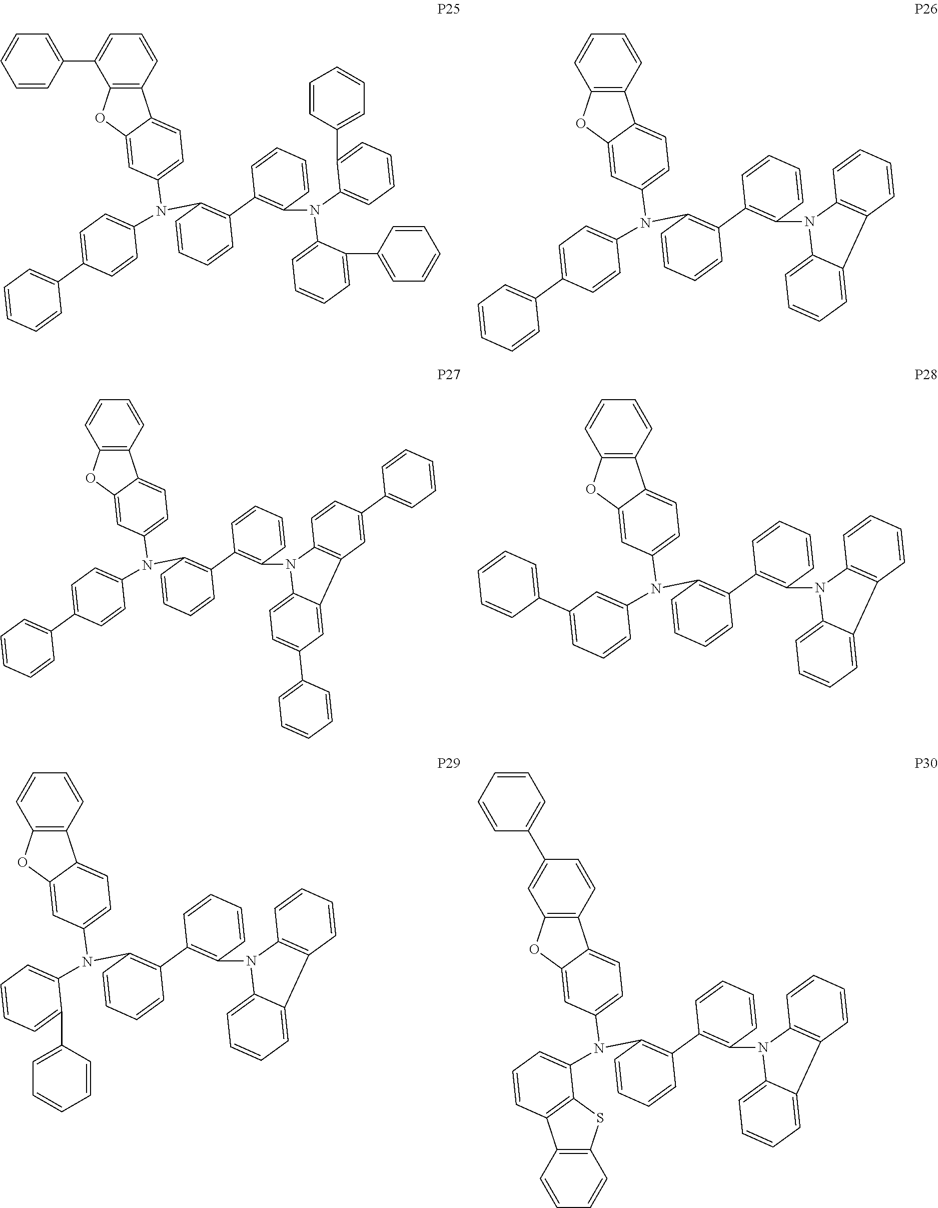 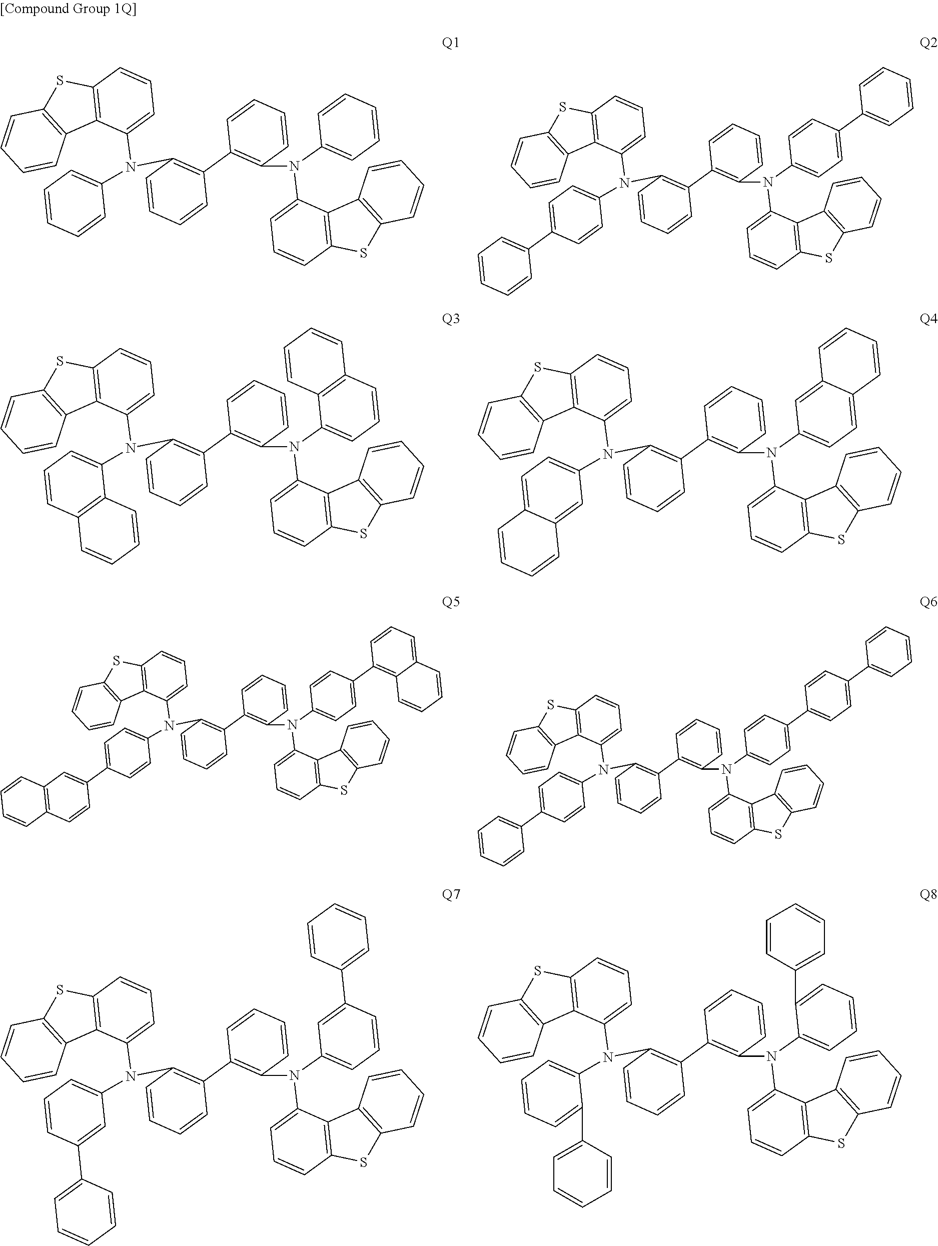 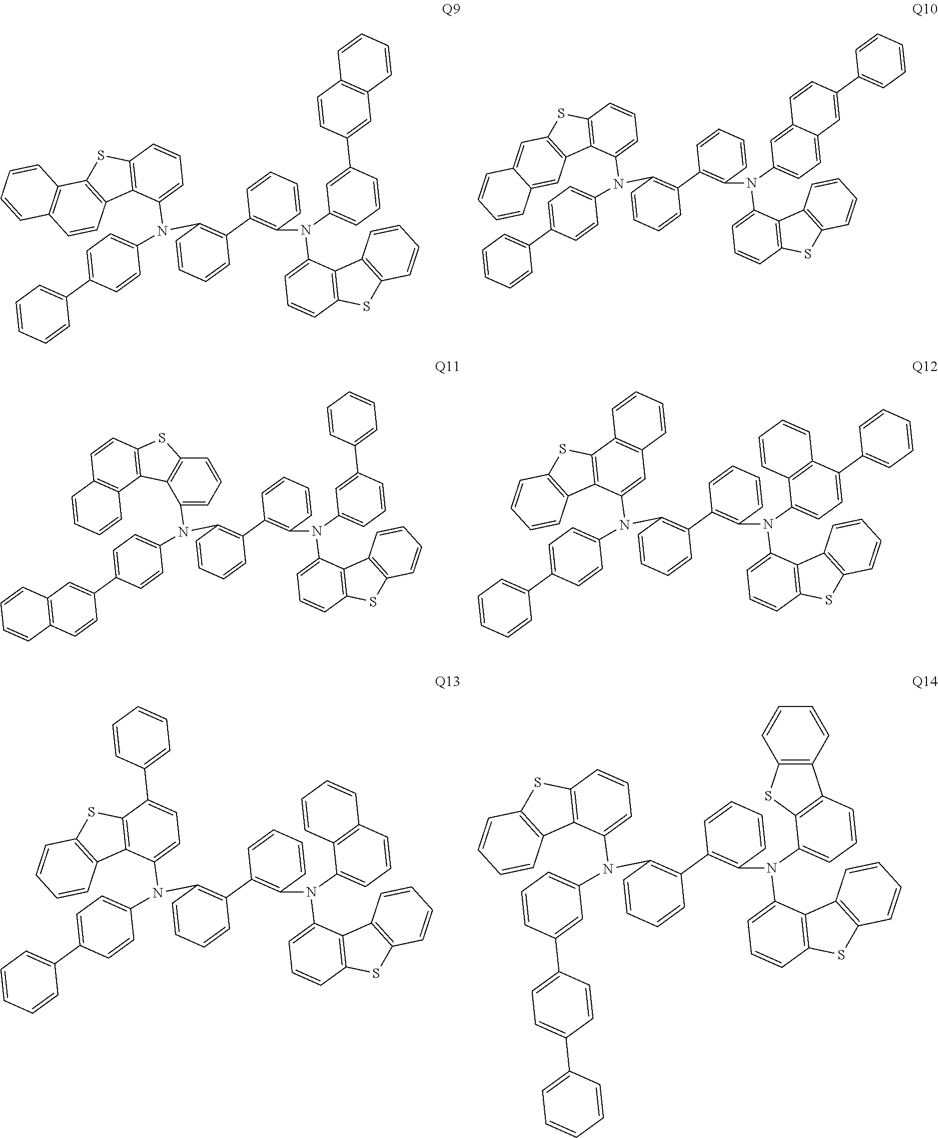 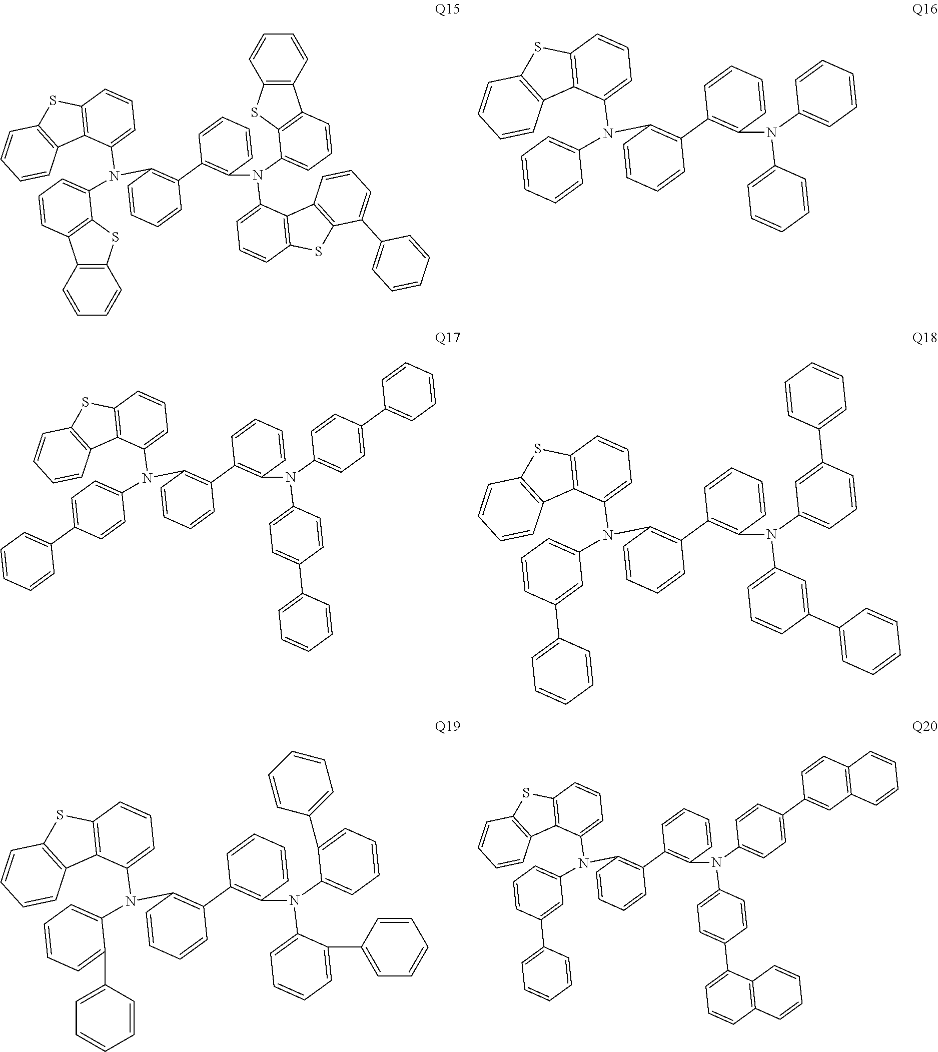 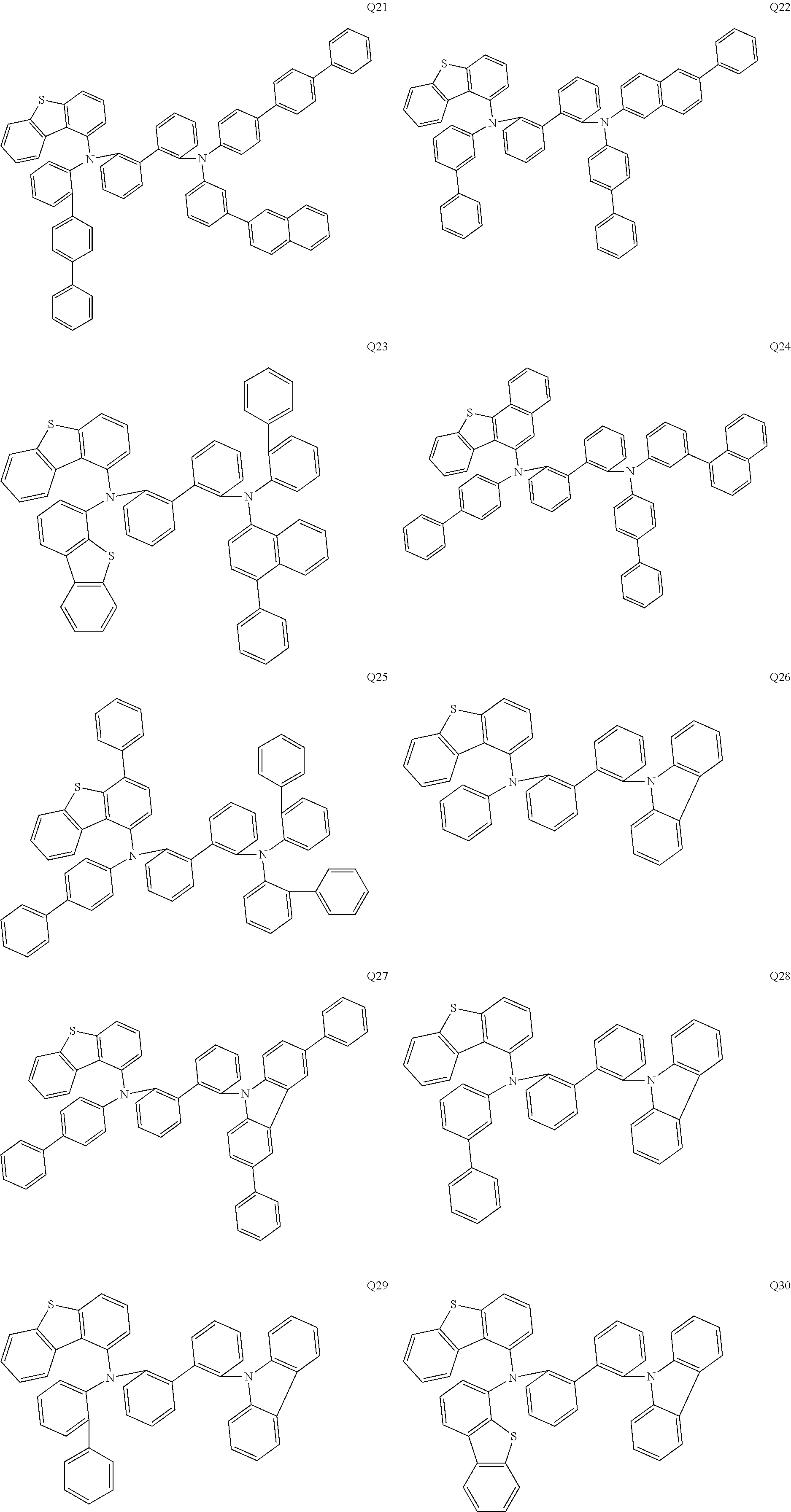 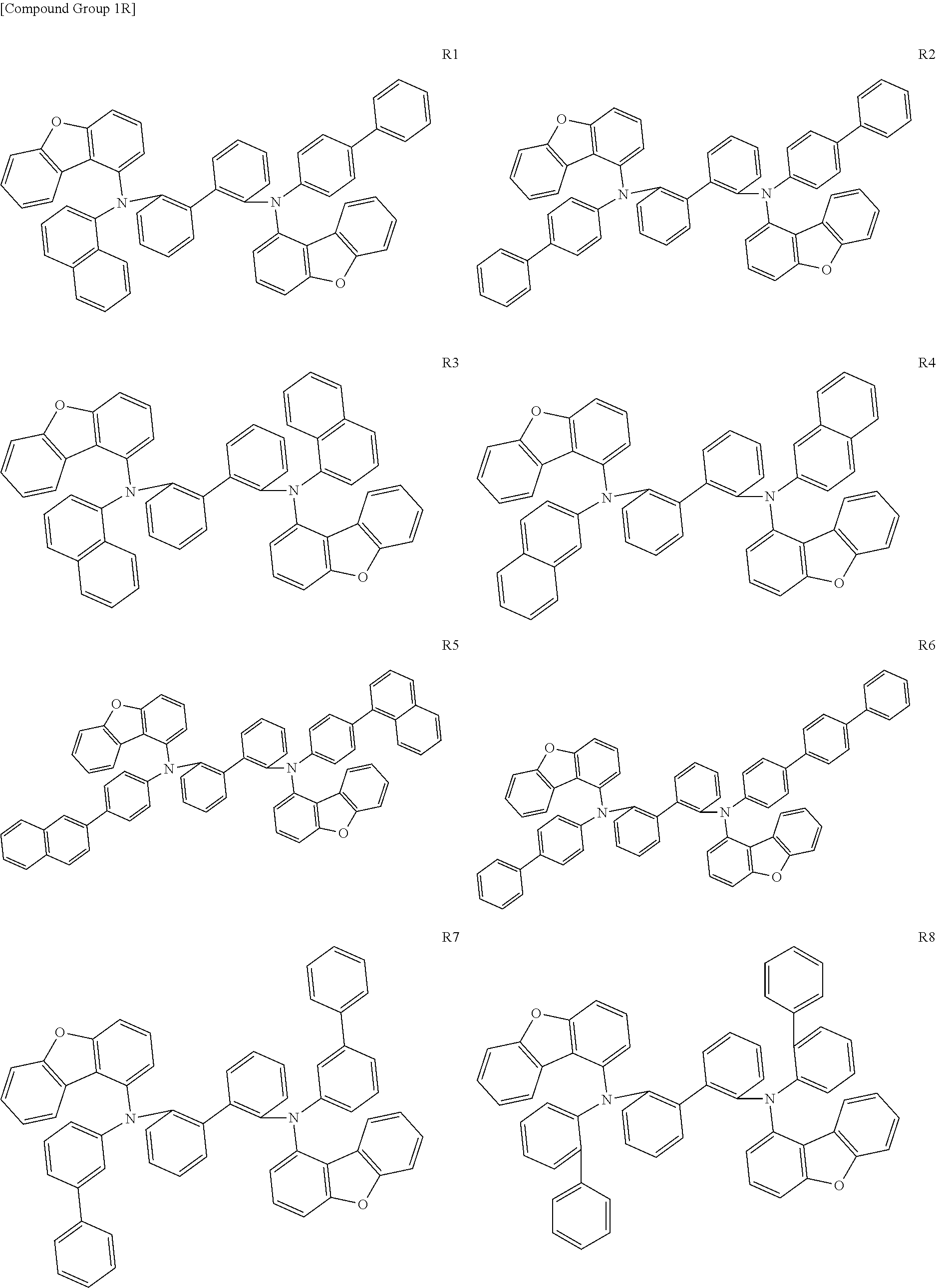 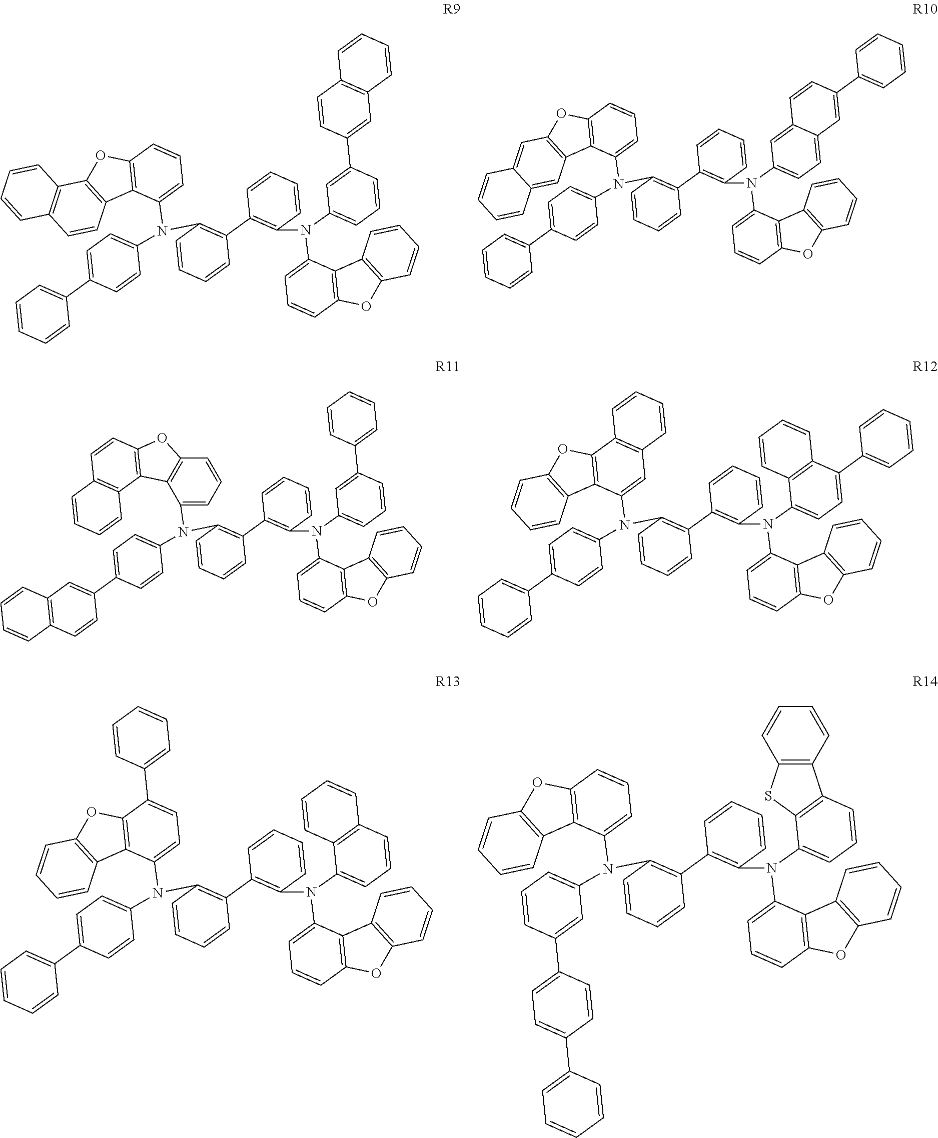 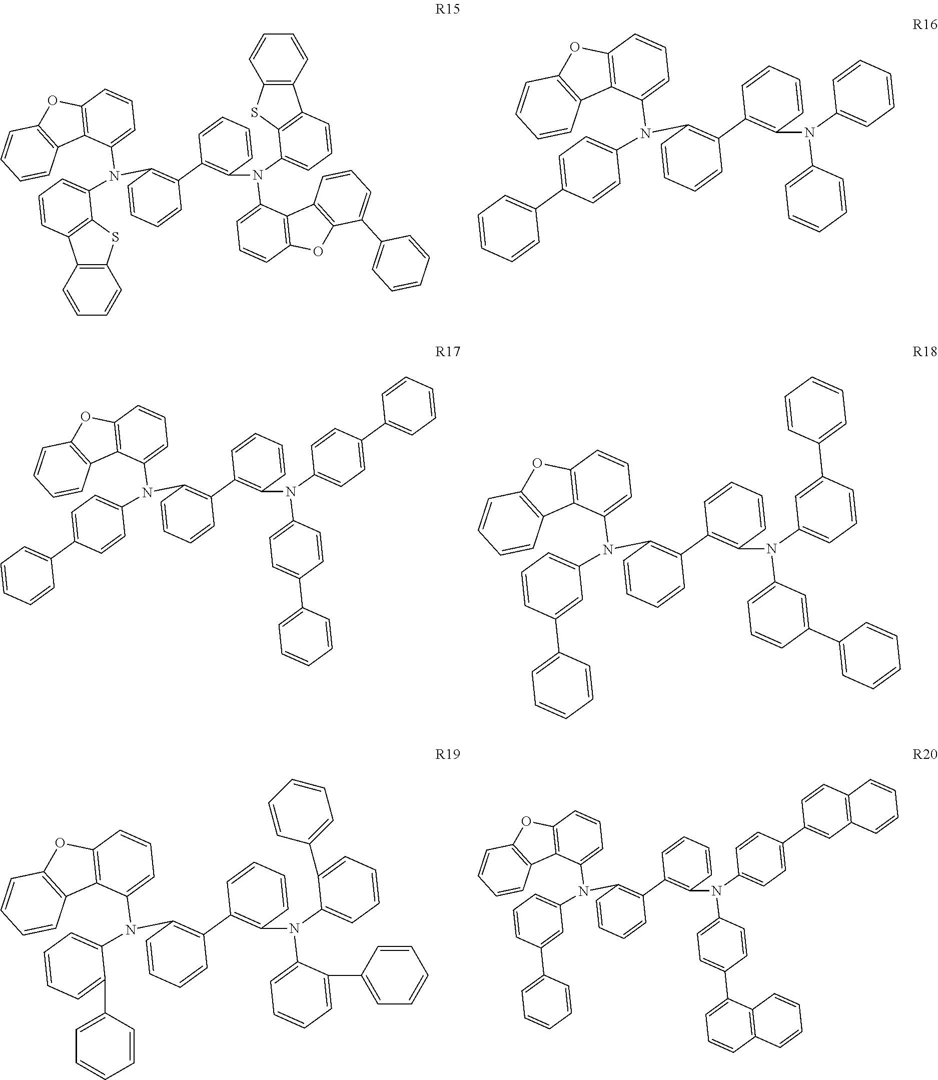 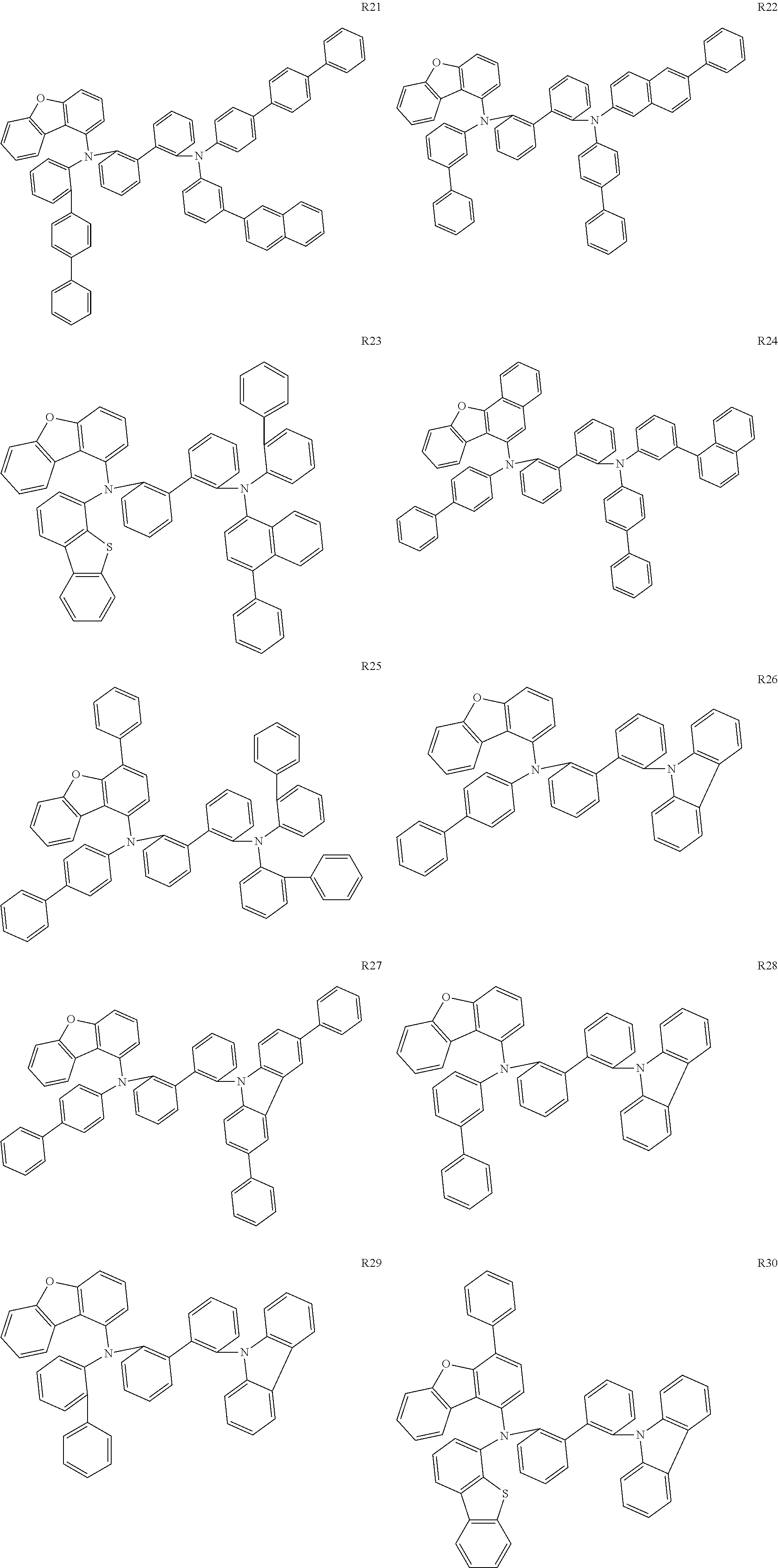 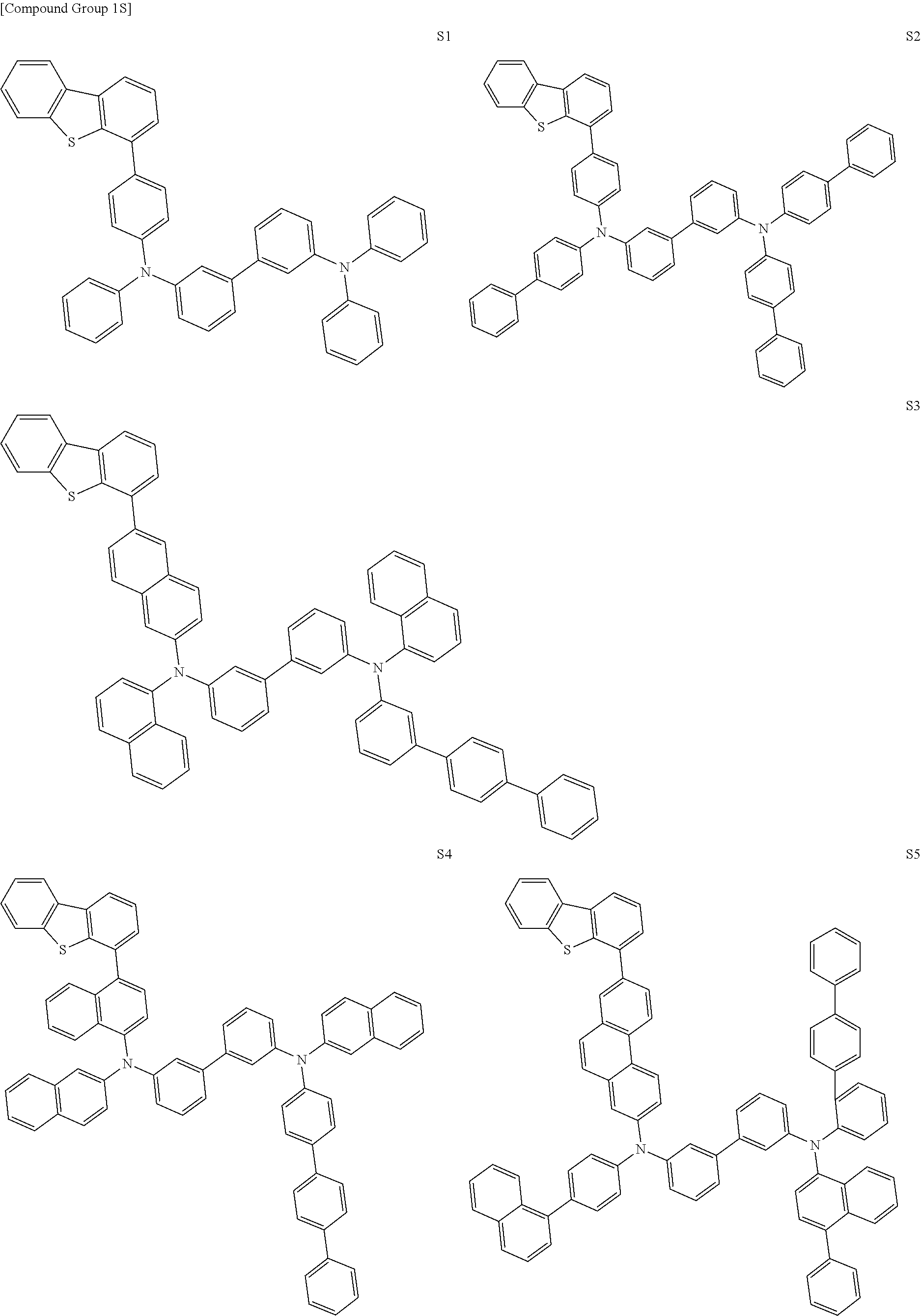 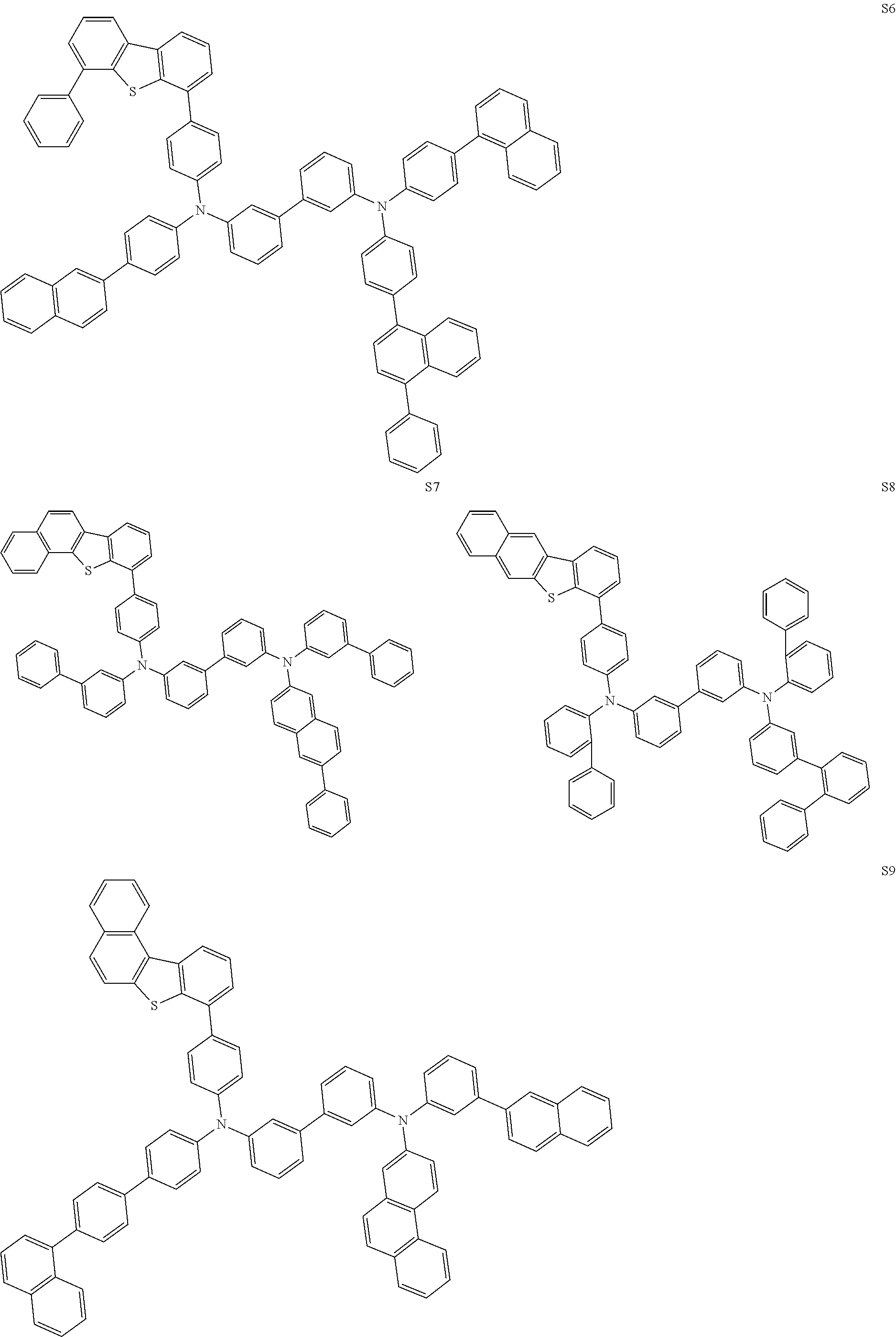 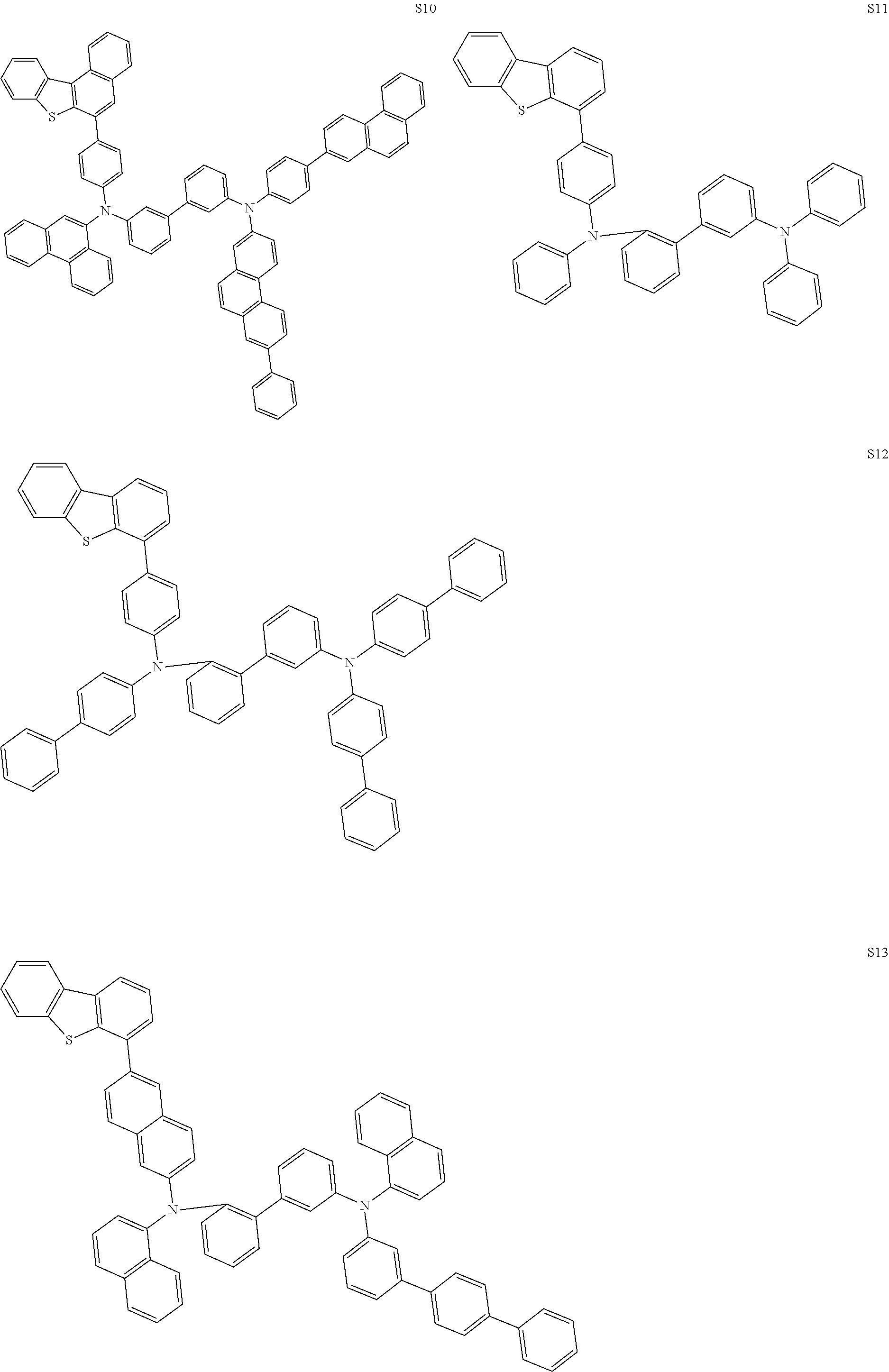 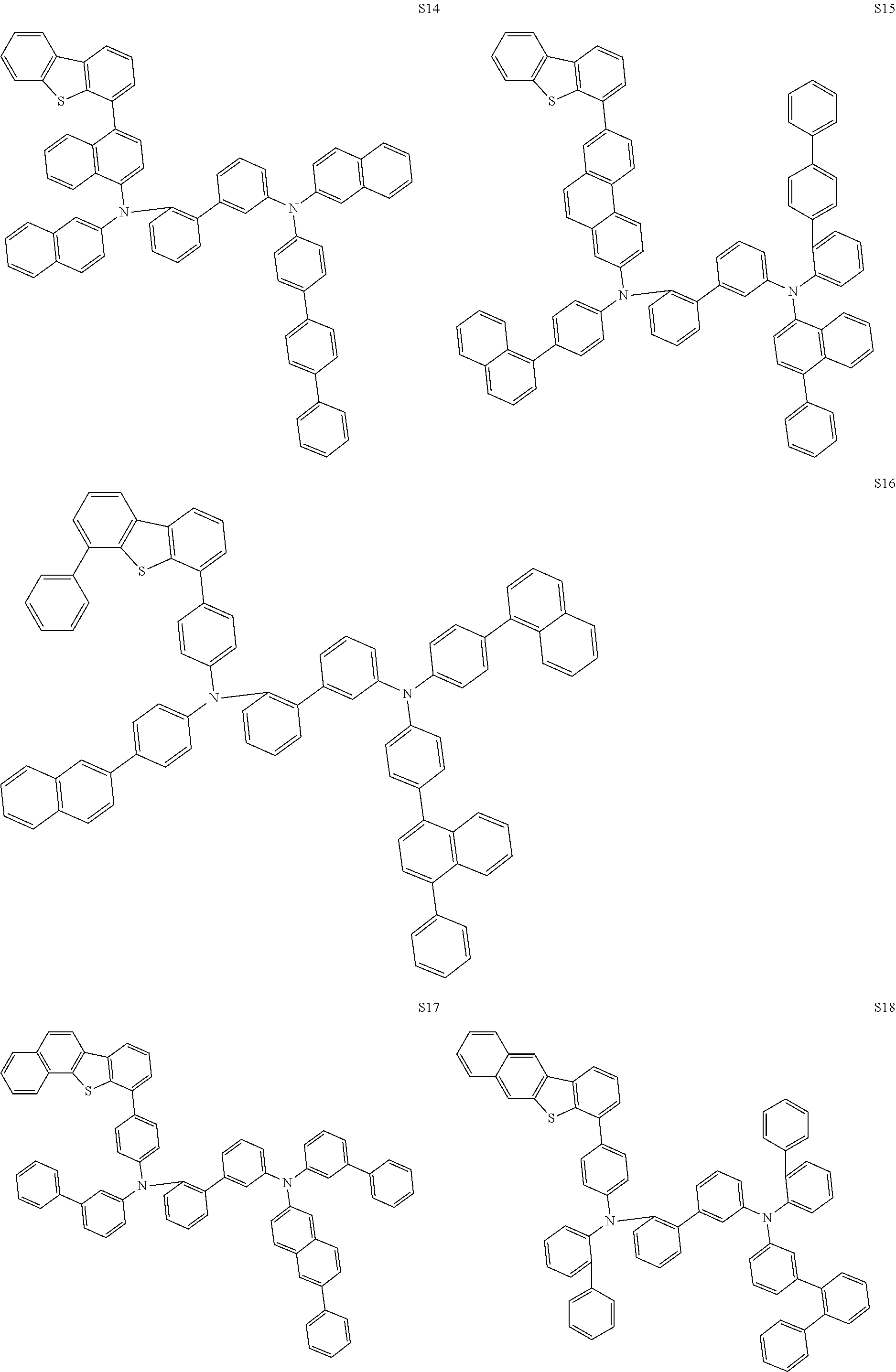 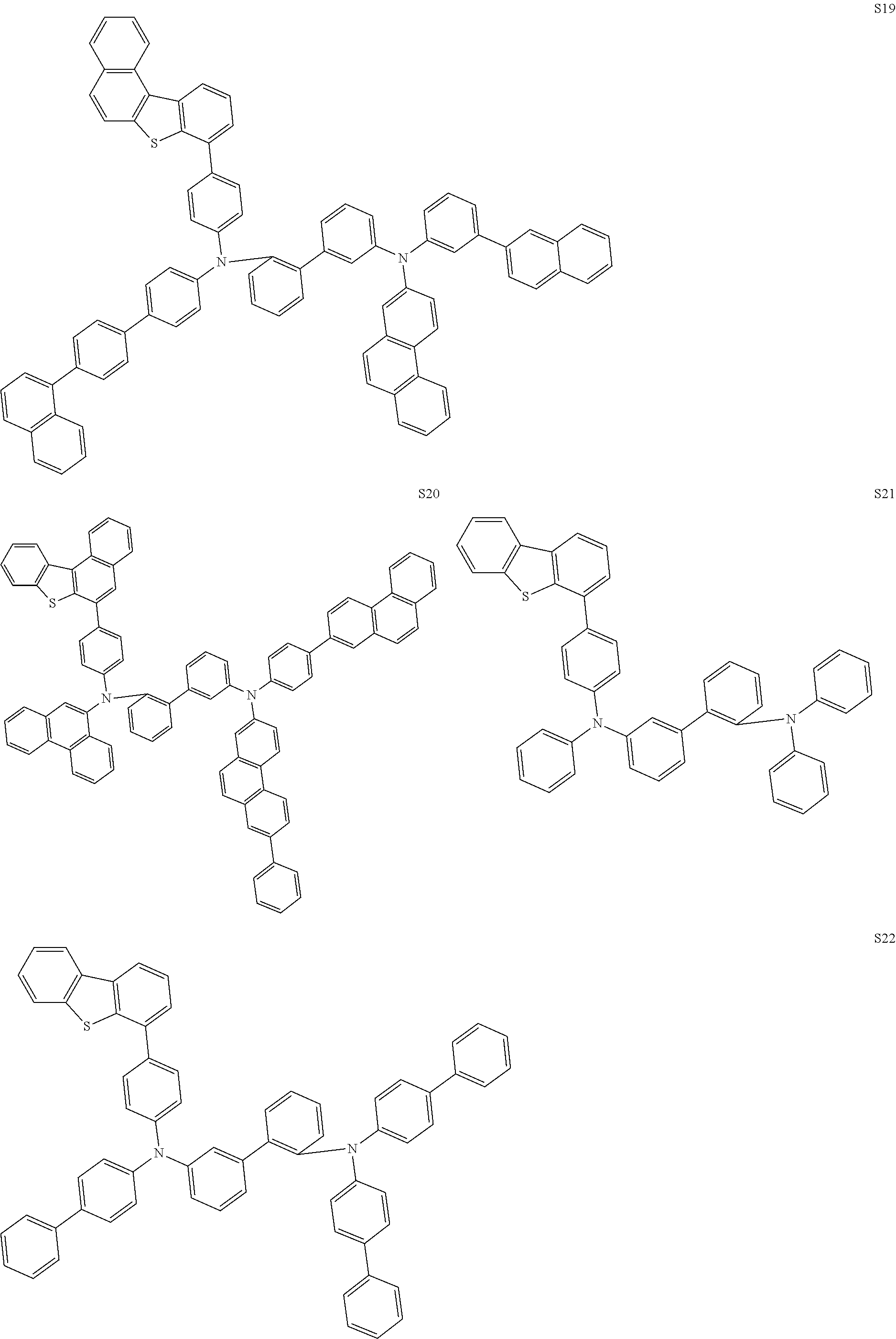 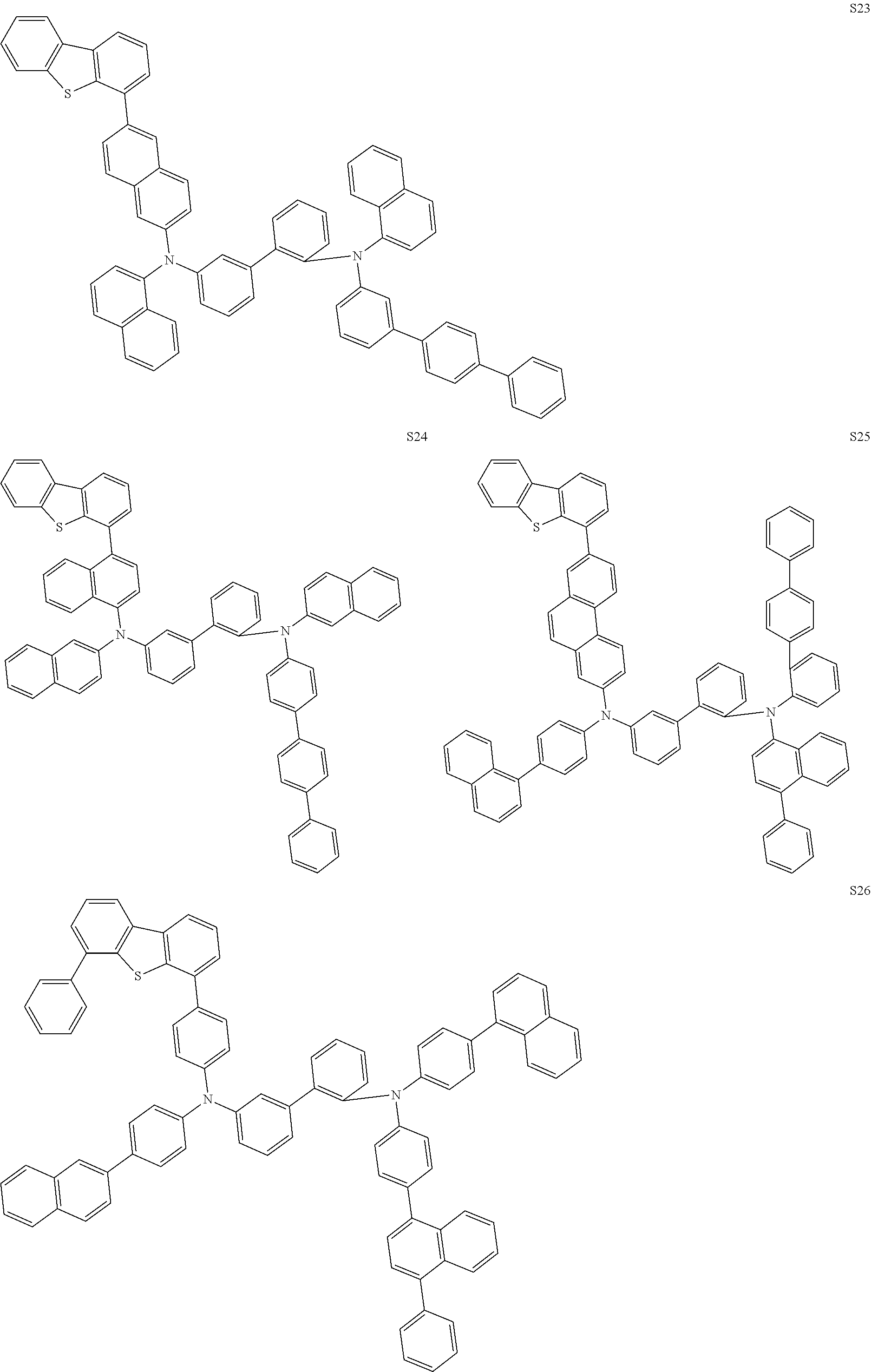 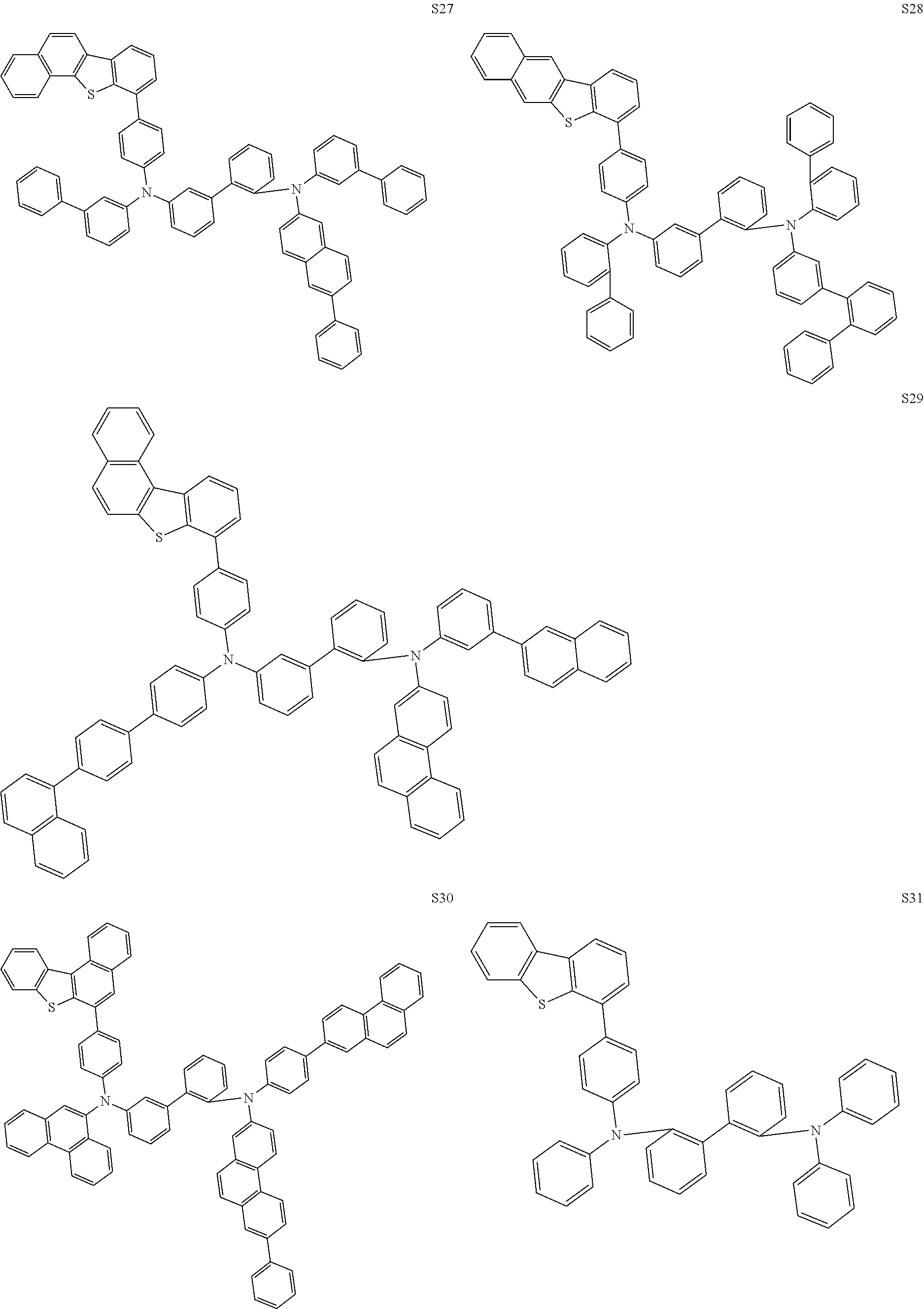 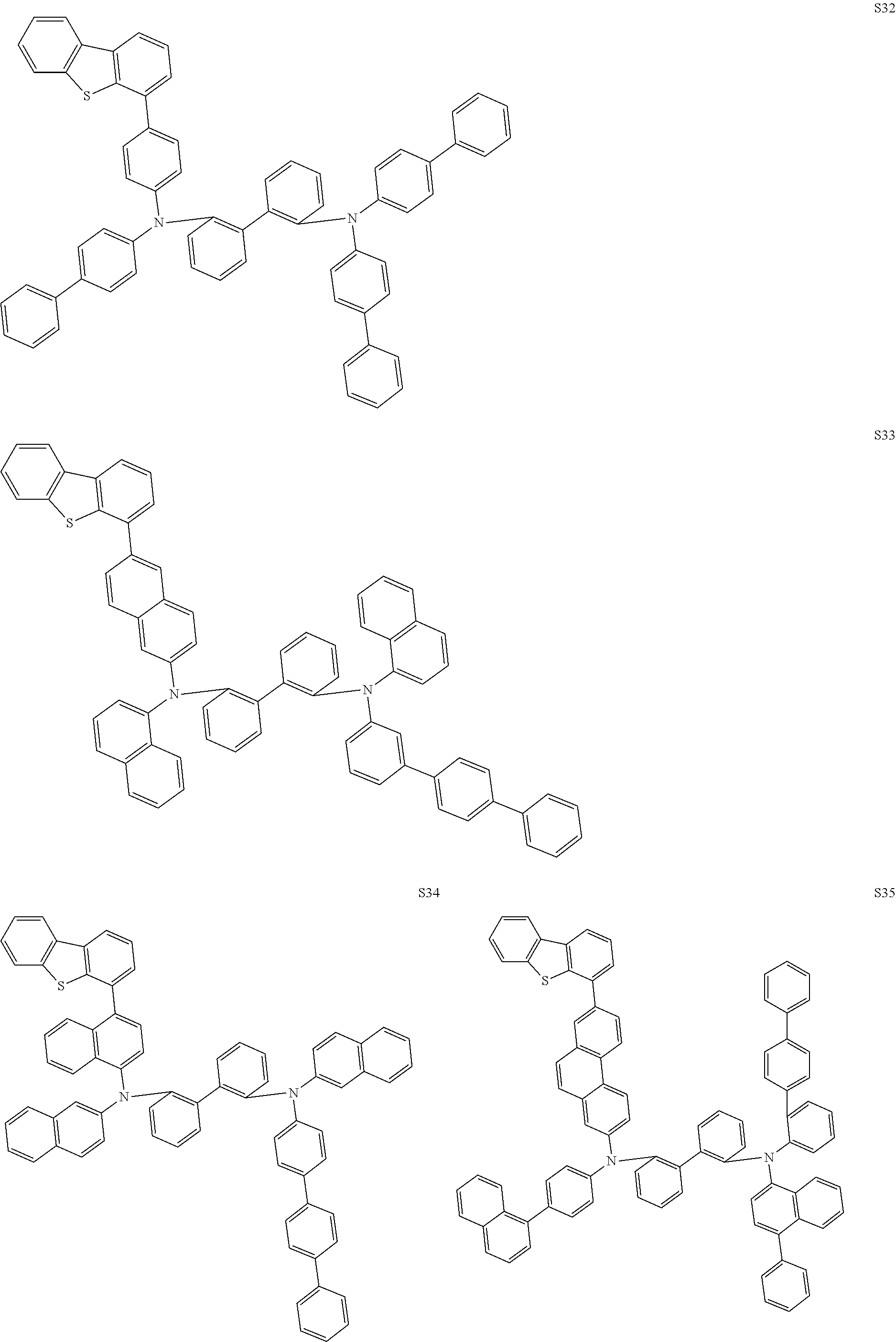 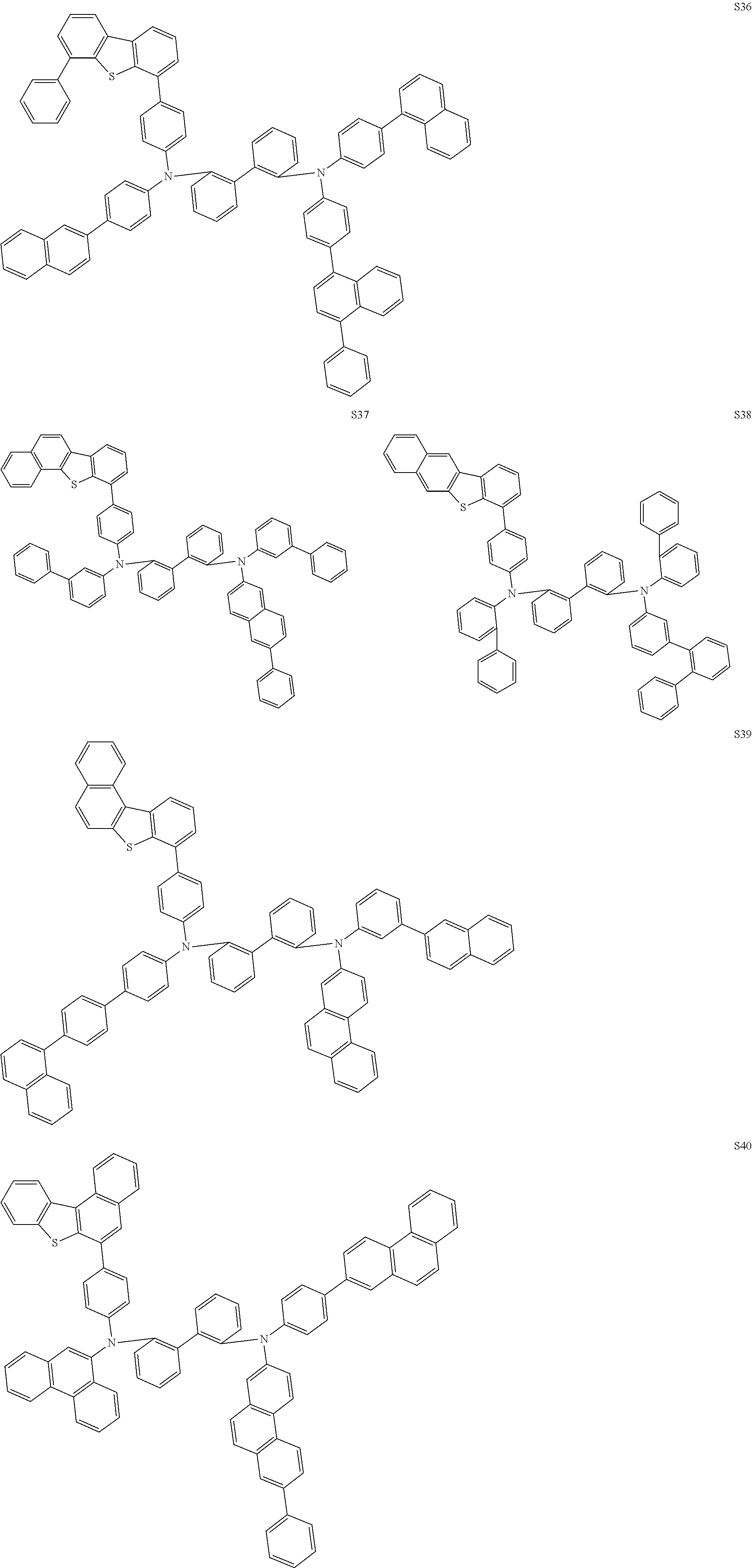 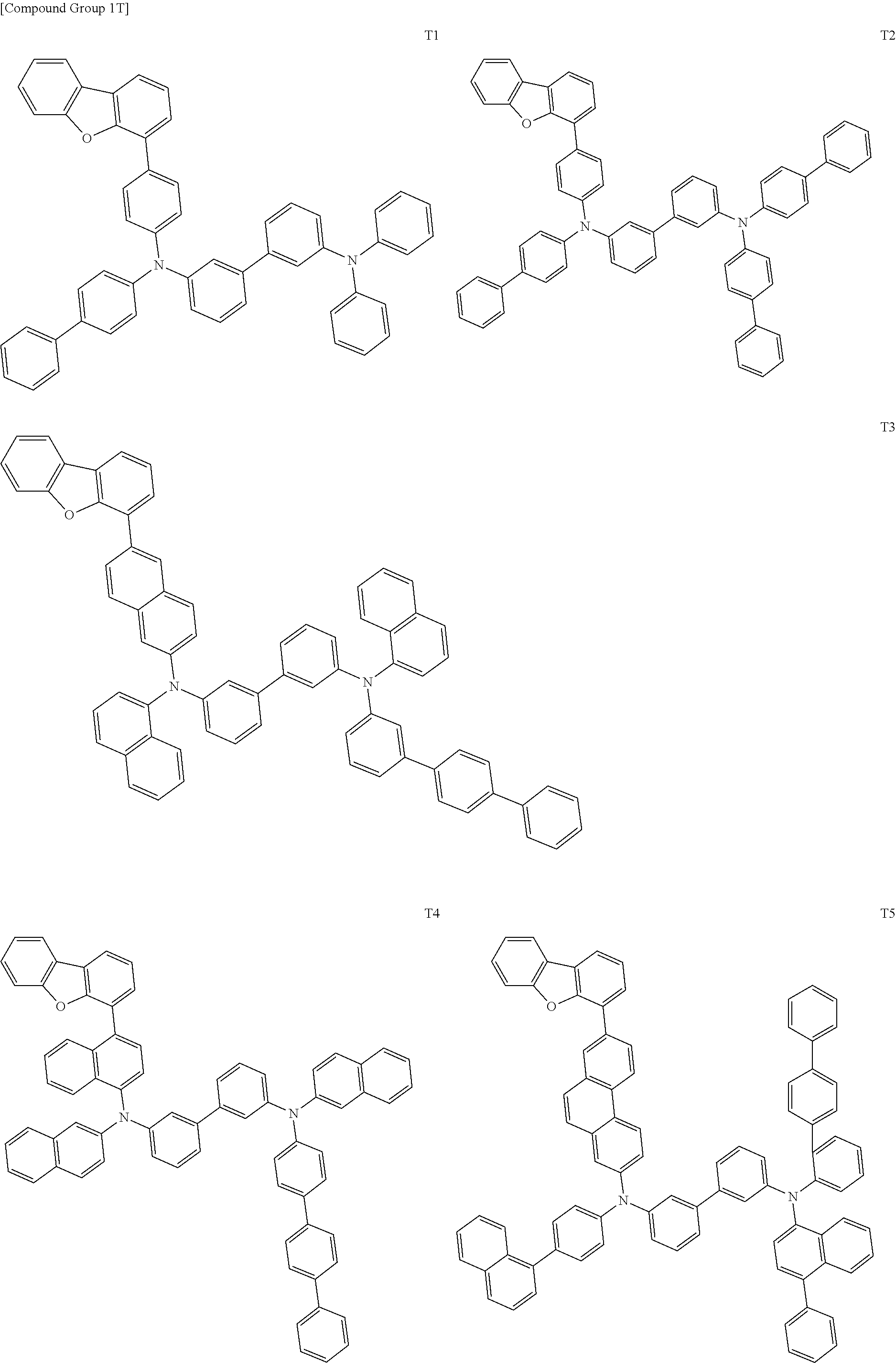 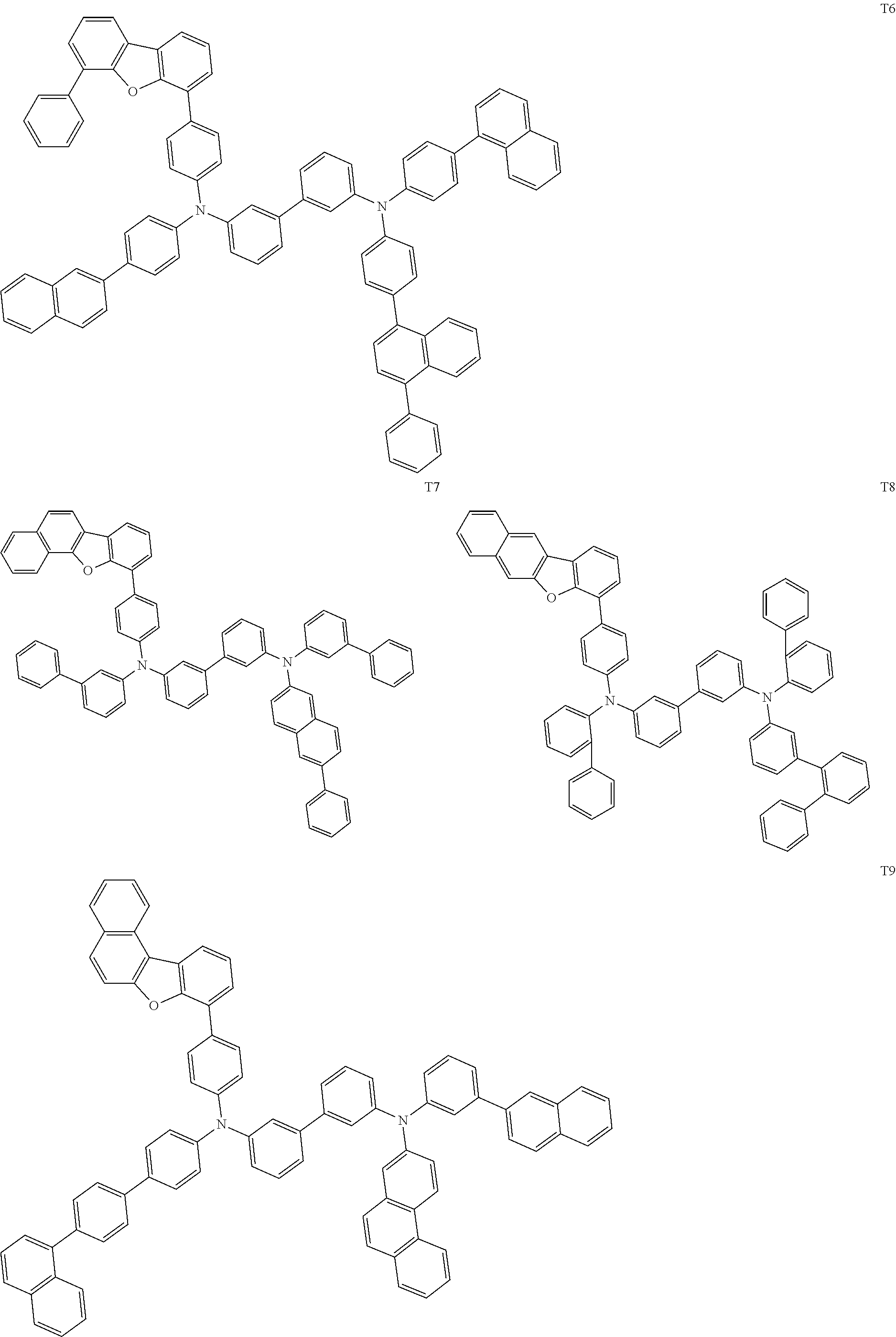 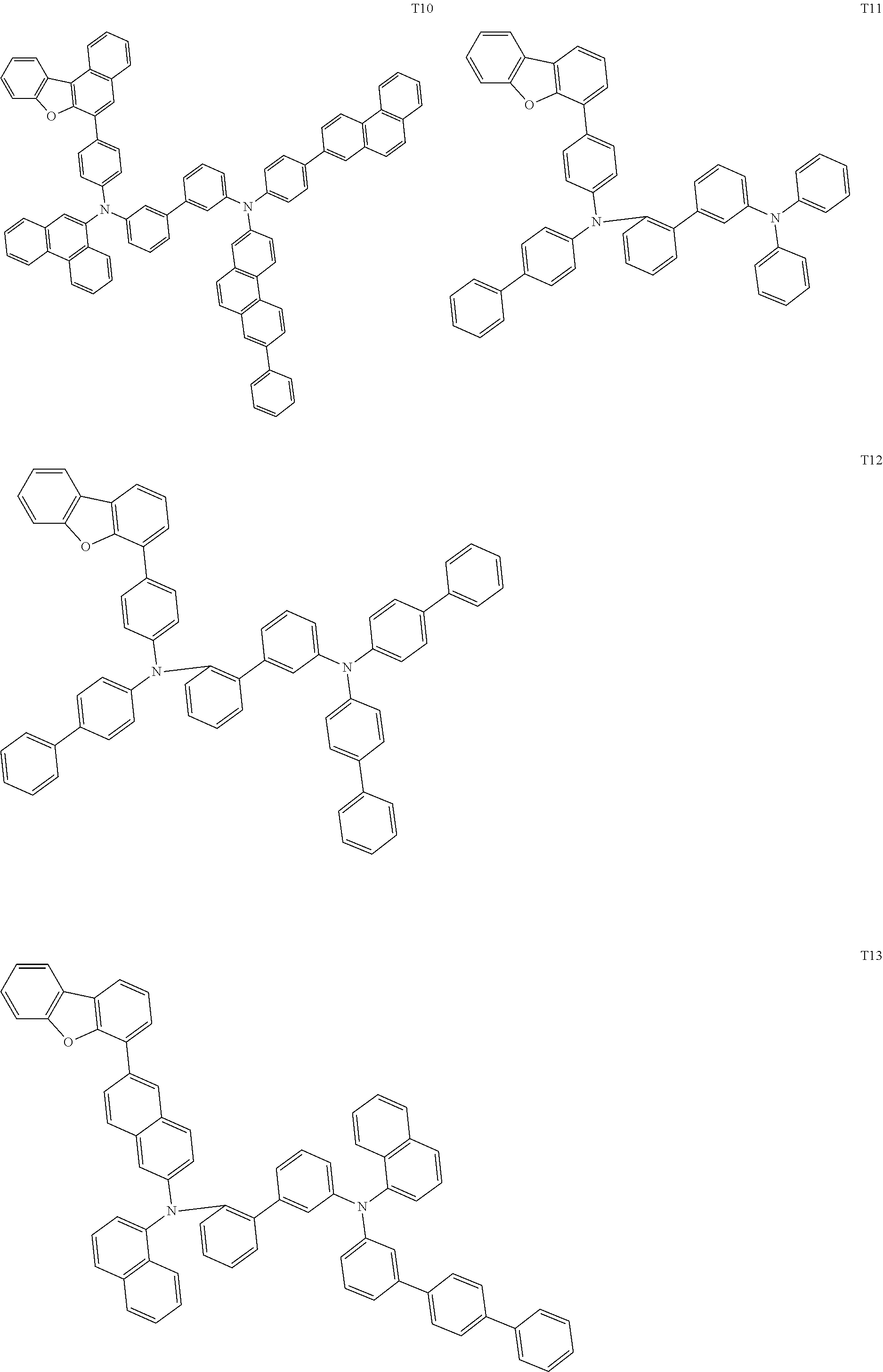 [SMILES: c1ccc(-c2ccc3c(ccc4cc(N(c5ccc(-c6ccc7c(ccc8ccccc87)c6)cc5)c5cccc(-c6cccc(N(c7ccc(-c8cc9ccccc9c9c8oc8ccccc89)cc7)c7cc8ccccc8c8ccccc78)c6)c5)ccc43)c2)cc1 ; c1ccc(-c2ccc(N(c3ccc(-c4cccc5c4oc4ccccc45)cc3)c3ccccc3-c3cccc(N(c4ccccc4)c4ccccc4)c3)cc2)cc1 ; c1ccc(-c2ccc(N(c3ccc(-c4ccccc4)cc3)c3cccc(-c4ccccc4N(c4ccc(-c5ccccc5)cc4)c4ccc(-c5cccc6c5oc5ccccc56)cc4)c3)cc2)cc1 ; c1ccc(-c2ccc(-c3cccc(N(c4cccc(-c5ccccc5N(c5ccc6cc(-c7cccc8c7oc7ccccc78)ccc6c5)c5cccc6ccccc56)c4)c4cccc5ccccc45)c3)cc2)cc1] 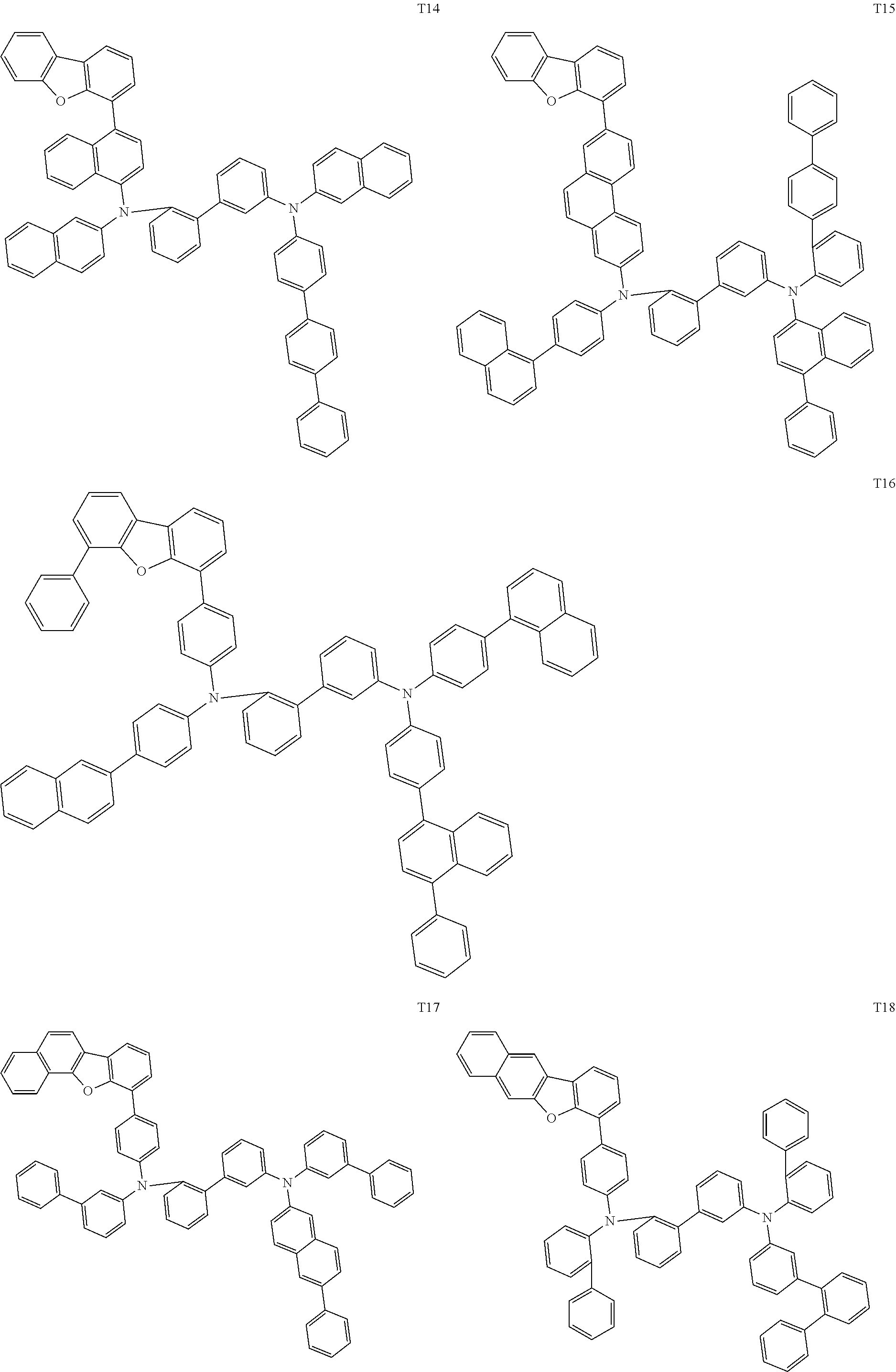 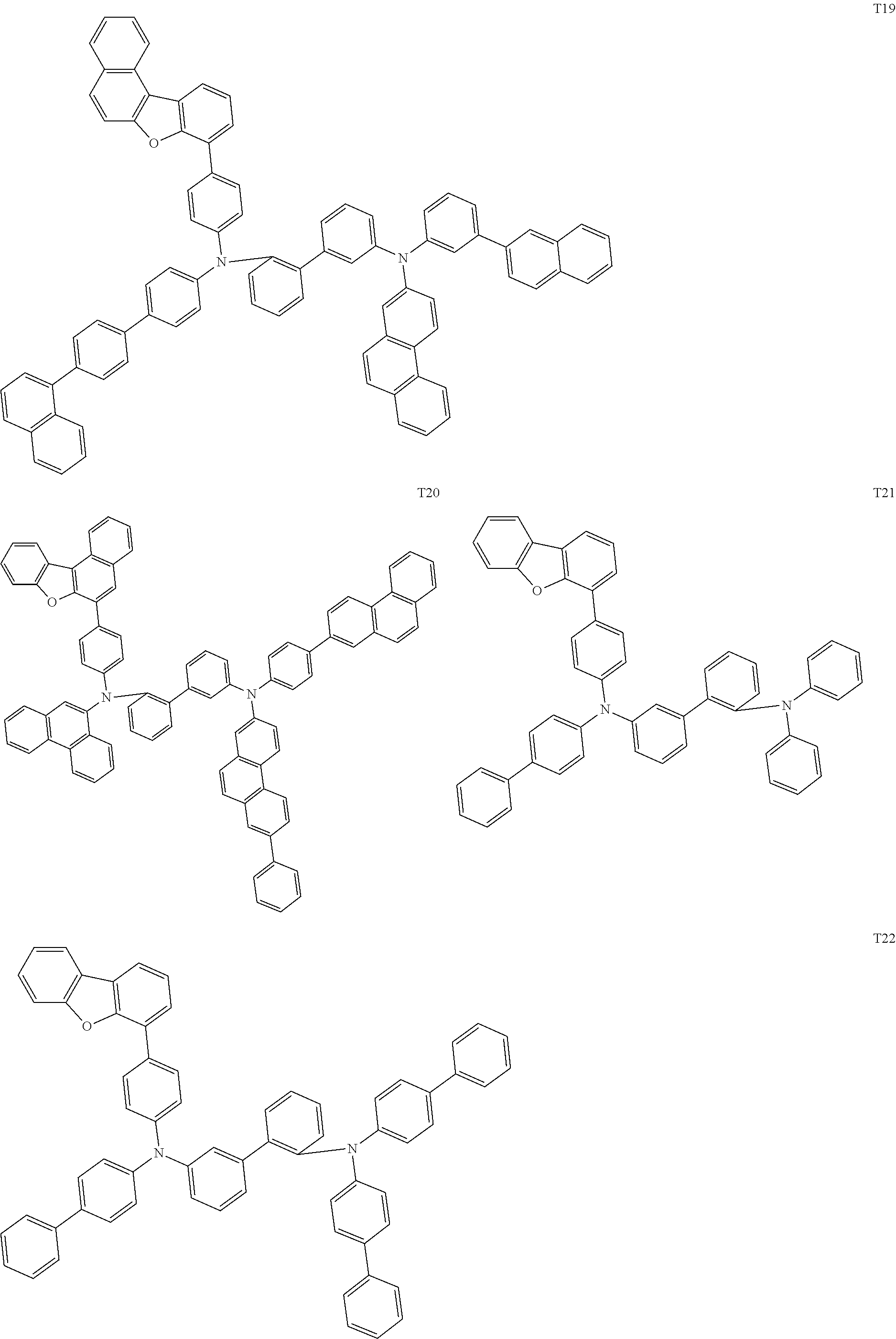 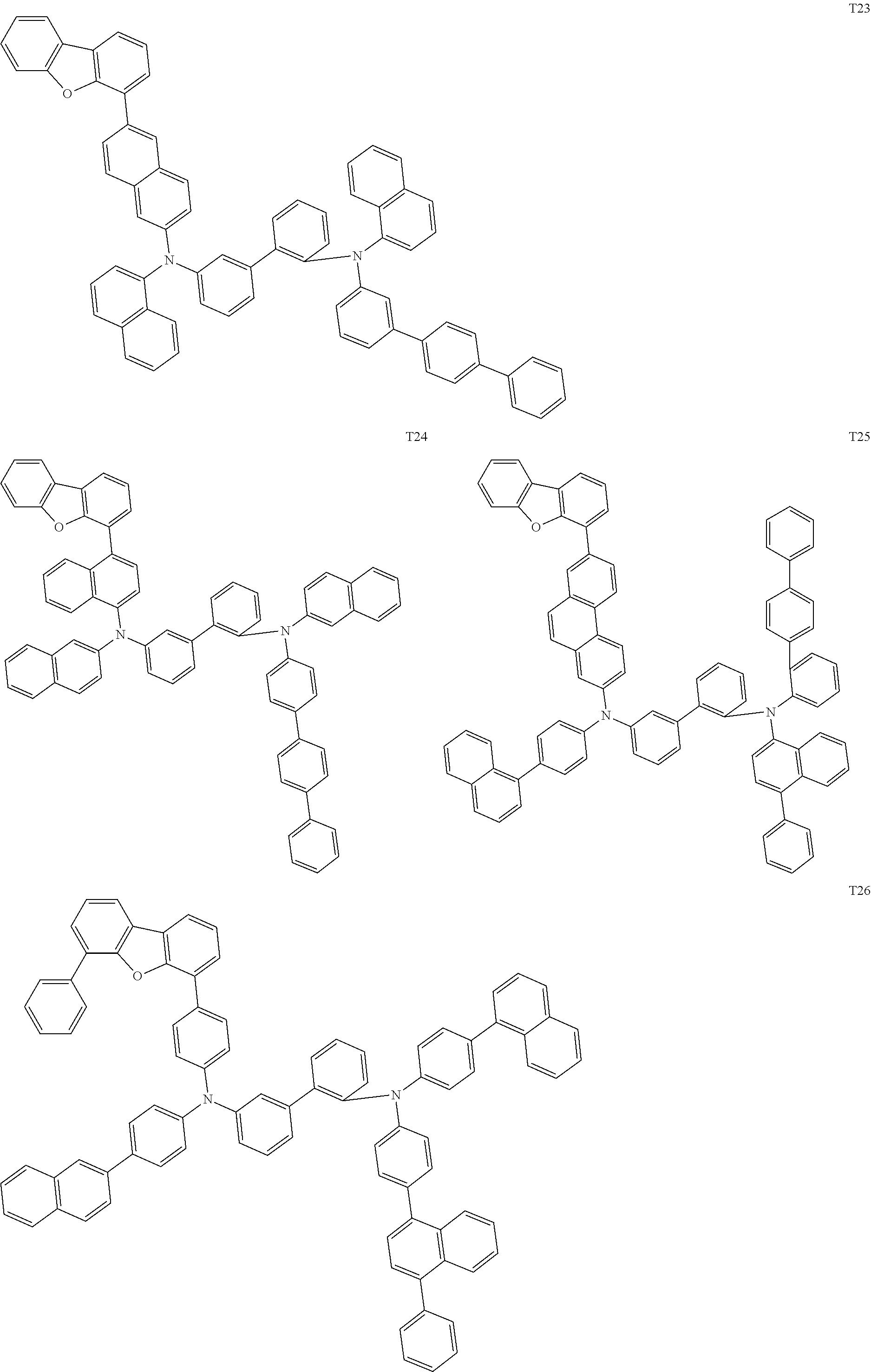 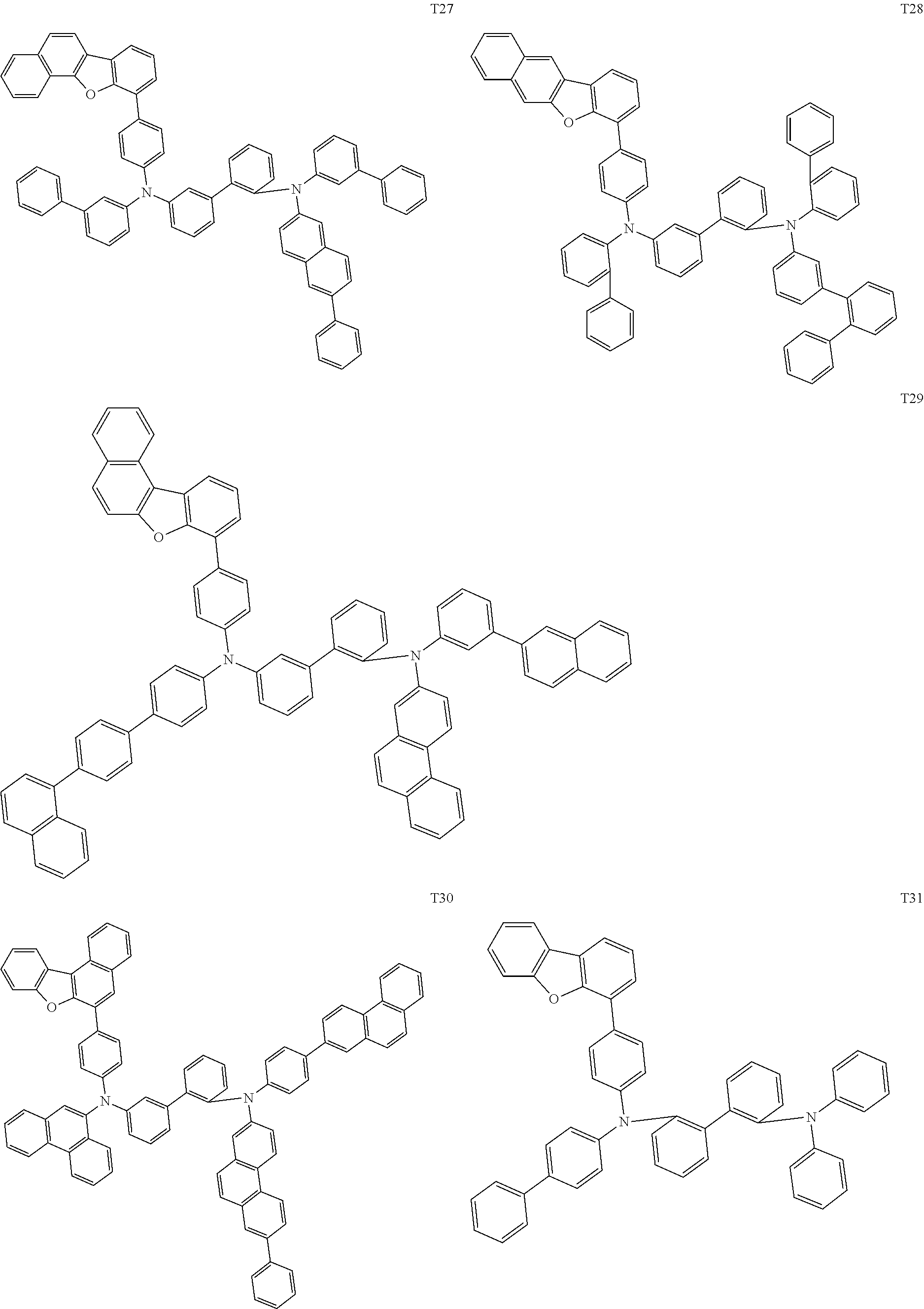 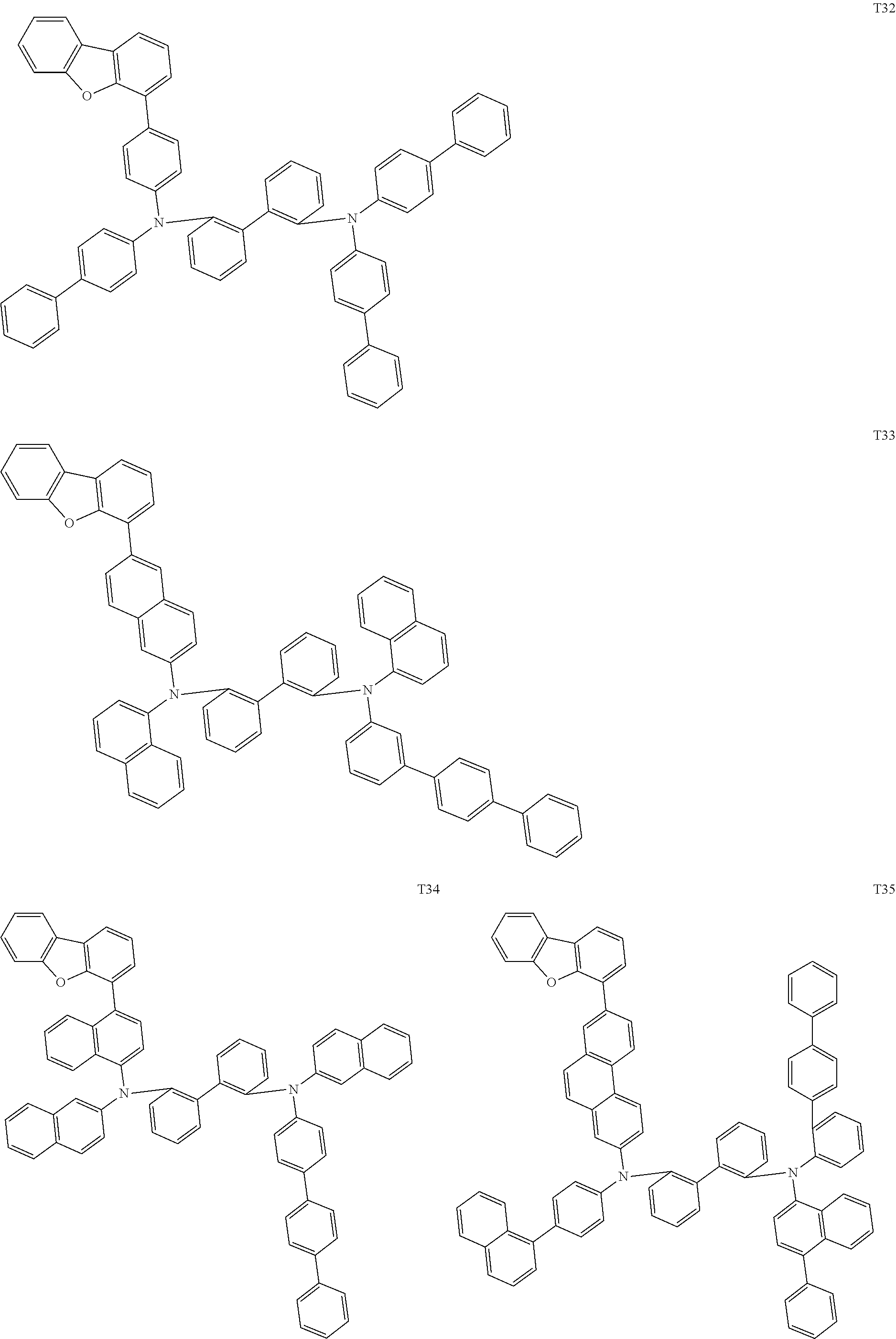 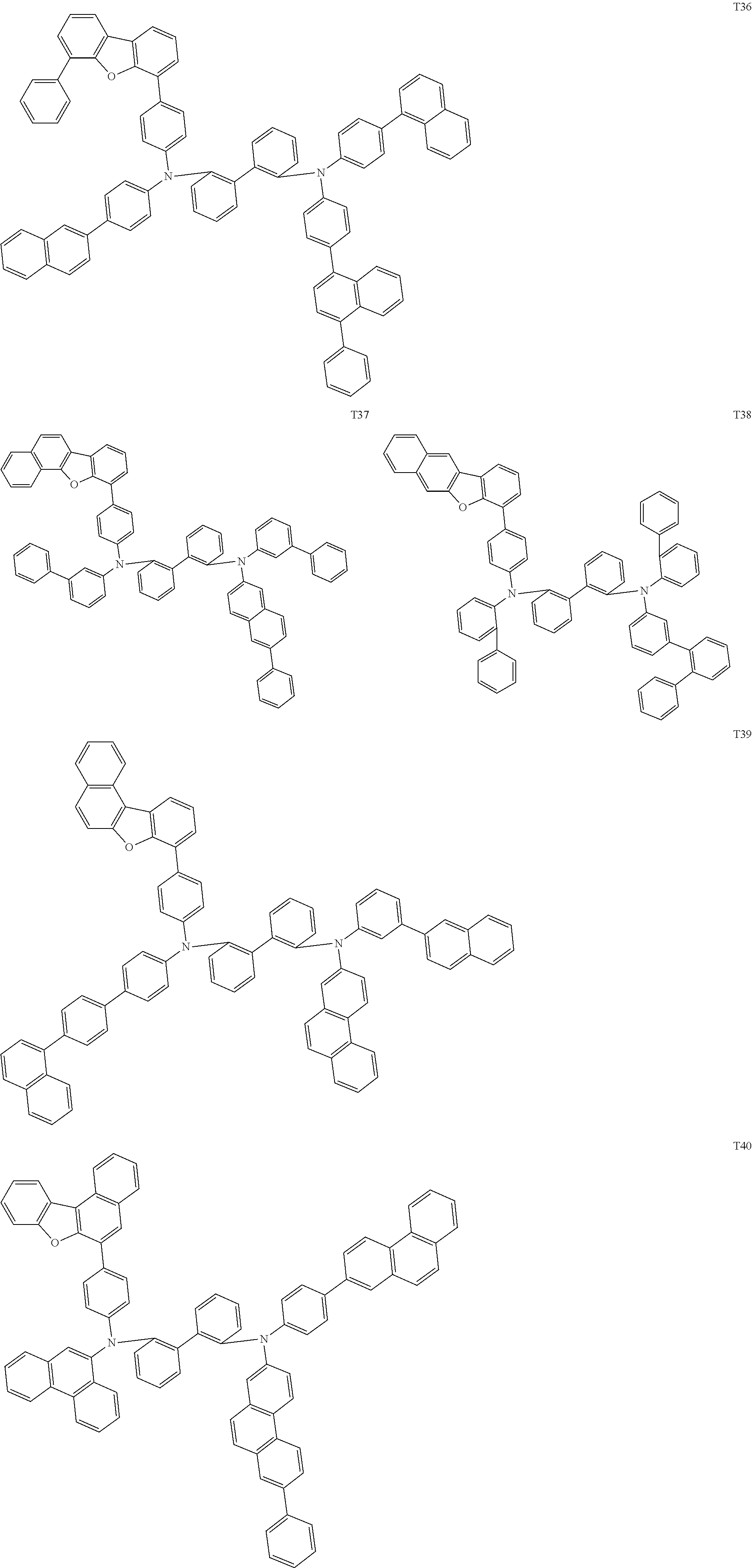 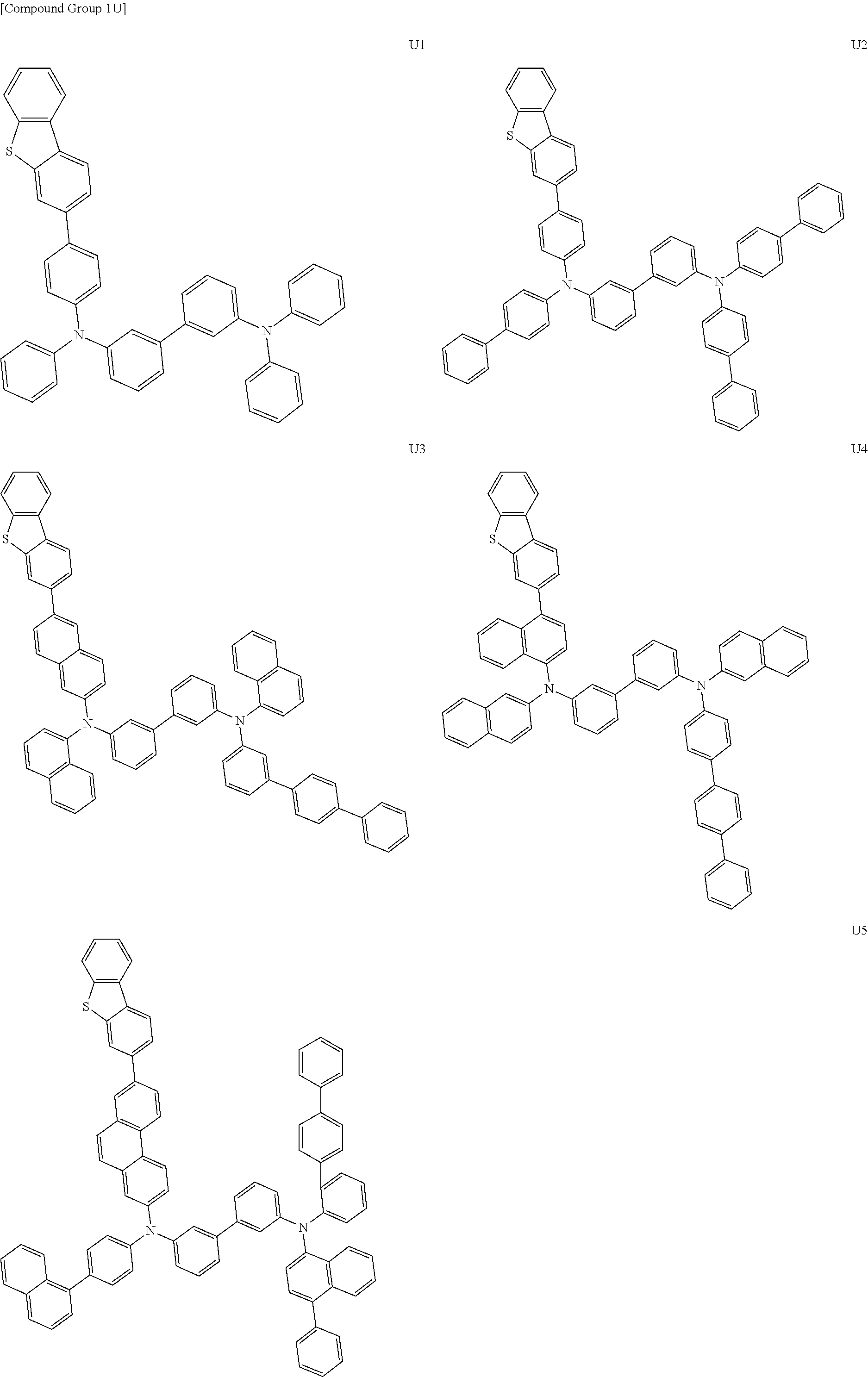 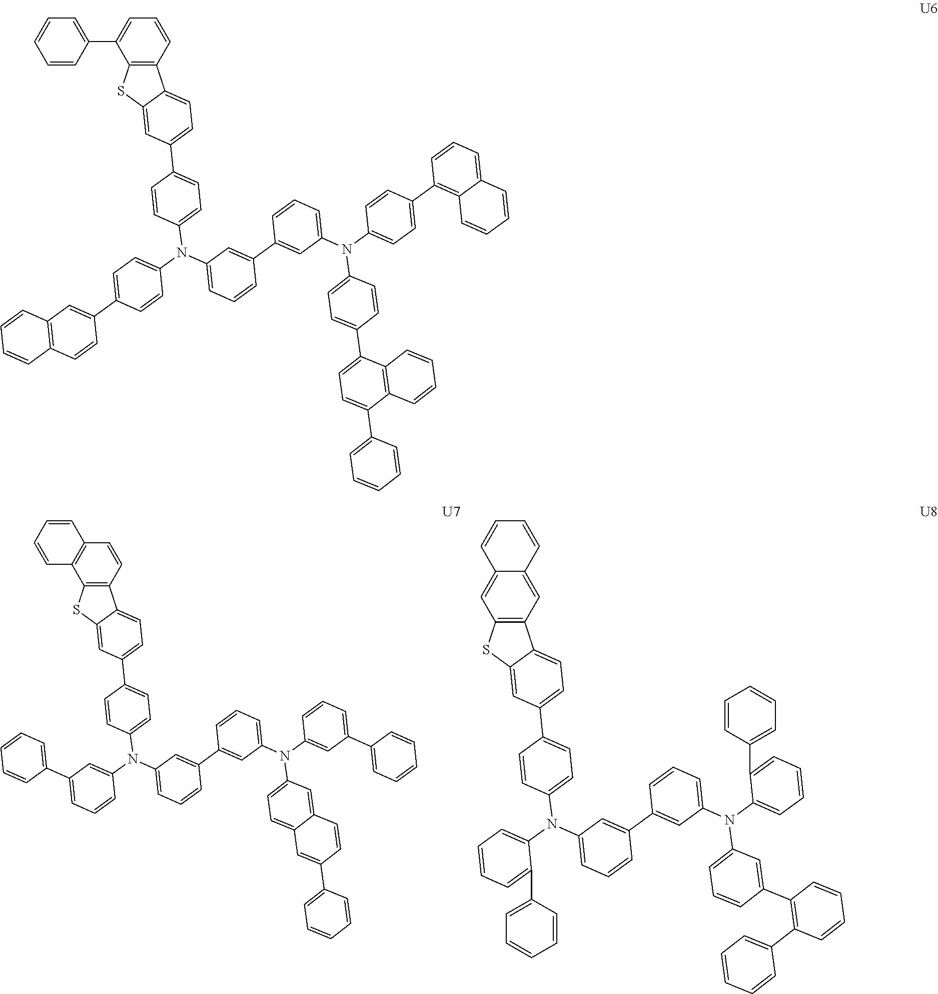 [SMILES: c1ccc(-c2ccc(-c3ccc(N(c4ccc(-c5cccc6ccccc56)cc4)c4cccc(-c5cccc(N(c6ccc(-c7ccc8ccccc8c7)cc6)c6ccc(-c7ccc8c(c7)sc7c(-c9ccccc9)cccc78)cc6)c5)c4)cc3)c3ccccc23)cc1 ; c1ccc(-c2cccc(N(c3ccc(-c4ccc5c(c4)sc4c6ccccc6ccc54)cc3)c3cccc(-c4cccc(N(c5cccc(-c6ccccc6)c5)c5ccc6cc(-c7ccccc7)ccc6c5)c4)c3)c2)cc1 ; c1ccc(-c2ccccc2-c2cccc(N(c3cccc(-c4cccc(N(c5ccc(-c6ccc7c(c6)sc6cc8ccccc8cc67)cc5)c5ccccc5-c5ccccc5)c4)c3)c3ccccc3-c3ccccc3)c2)cc1] 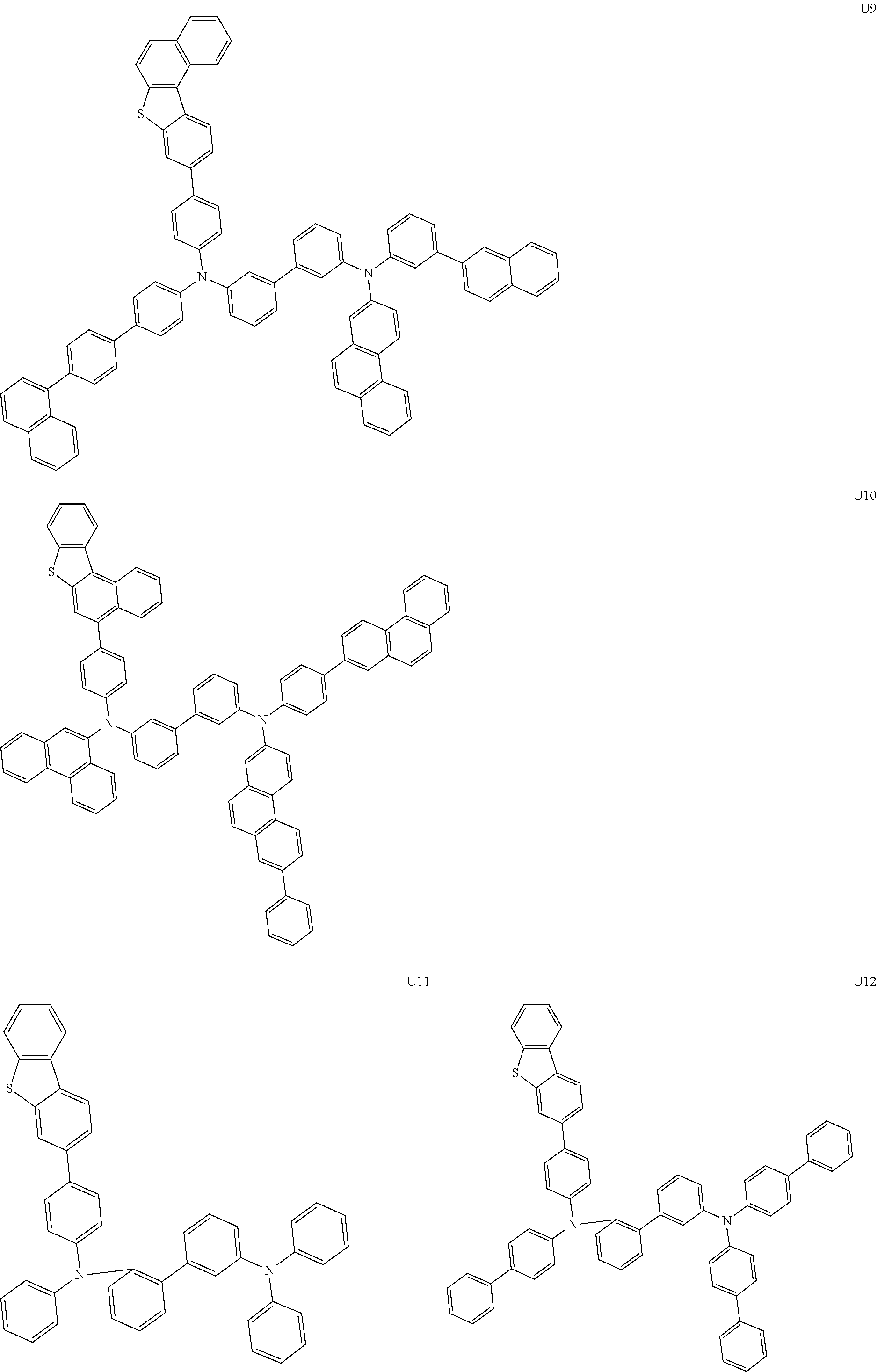 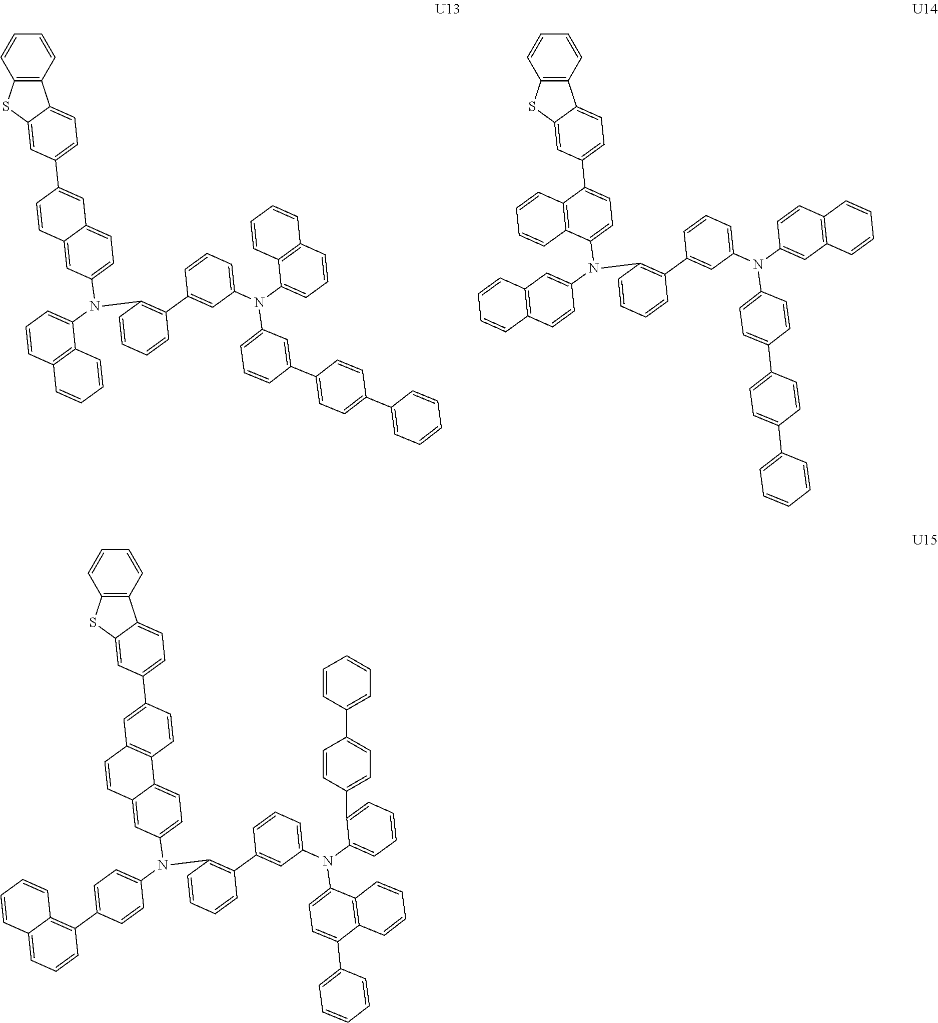 [SMILES: c1ccc(-c2ccc(-c3cccc(N(c4cccc(-c5ccccc5N(c5ccc6cc(-c7ccc8c(c7)sc7ccccc78)ccc6c5)c5cccc6ccccc56)c4)c4cccc5ccccc45)c3)cc2)cc1 ; c1ccc(-c2ccc(-c3ccc(N(c4cccc(-c5ccccc5N(c5ccc6ccccc6c5)c5ccc(-c6ccc7c(c6)sc6ccccc67)c6ccccc56)c4)c4ccc5ccccc5c4)cc3)cc2)cc1 ; c1ccc(-c2ccc(-c3ccccc3N(c3cccc(-c4ccccc4N(c4ccc(-c5cccc6ccccc56)cc4)c4ccc5c(ccc6cc(-c7ccc8c(c7)sc7ccccc78)ccc65)c4)c3)c3ccc(-c4ccccc4)c4ccccc34)cc2)cc1] 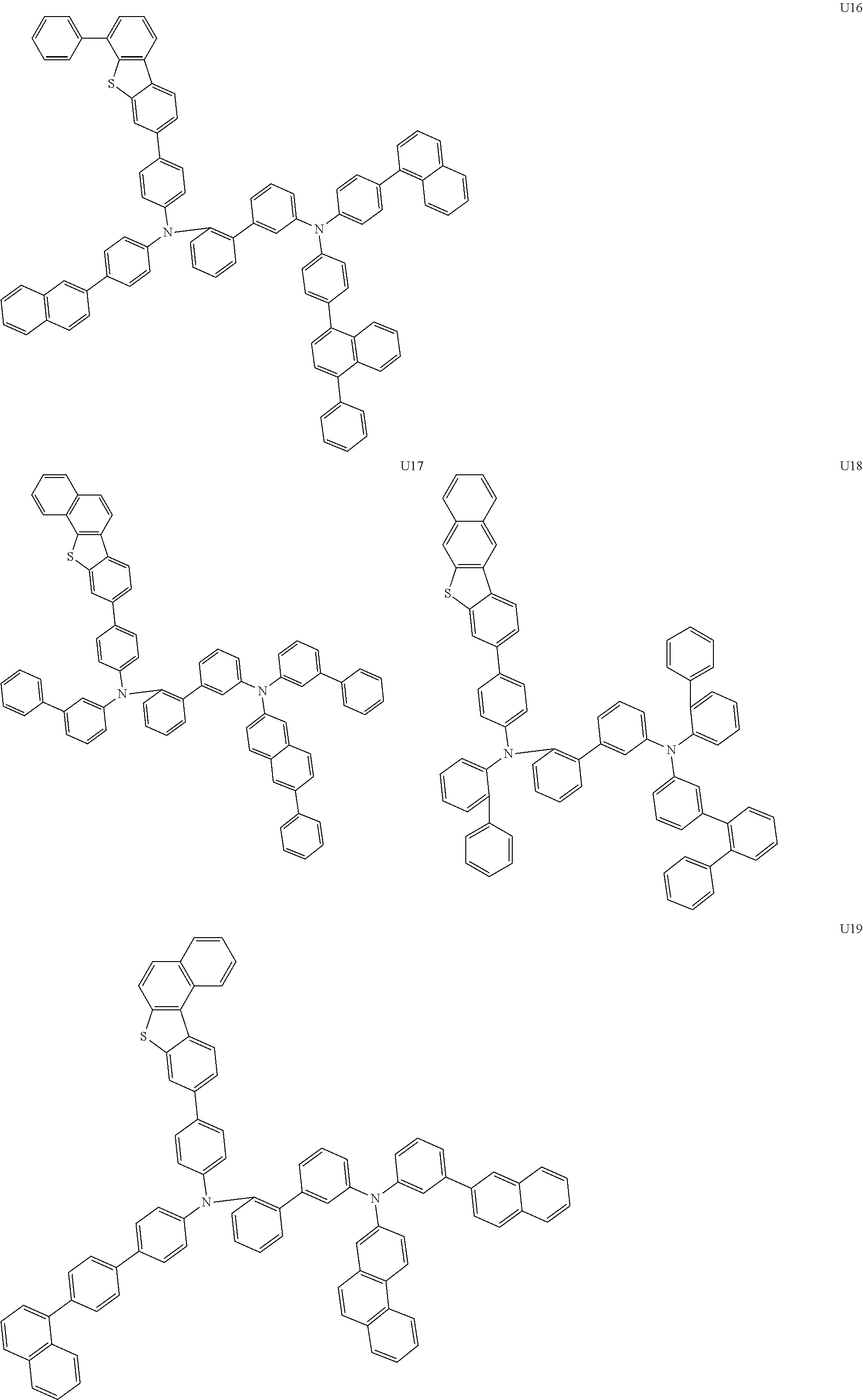 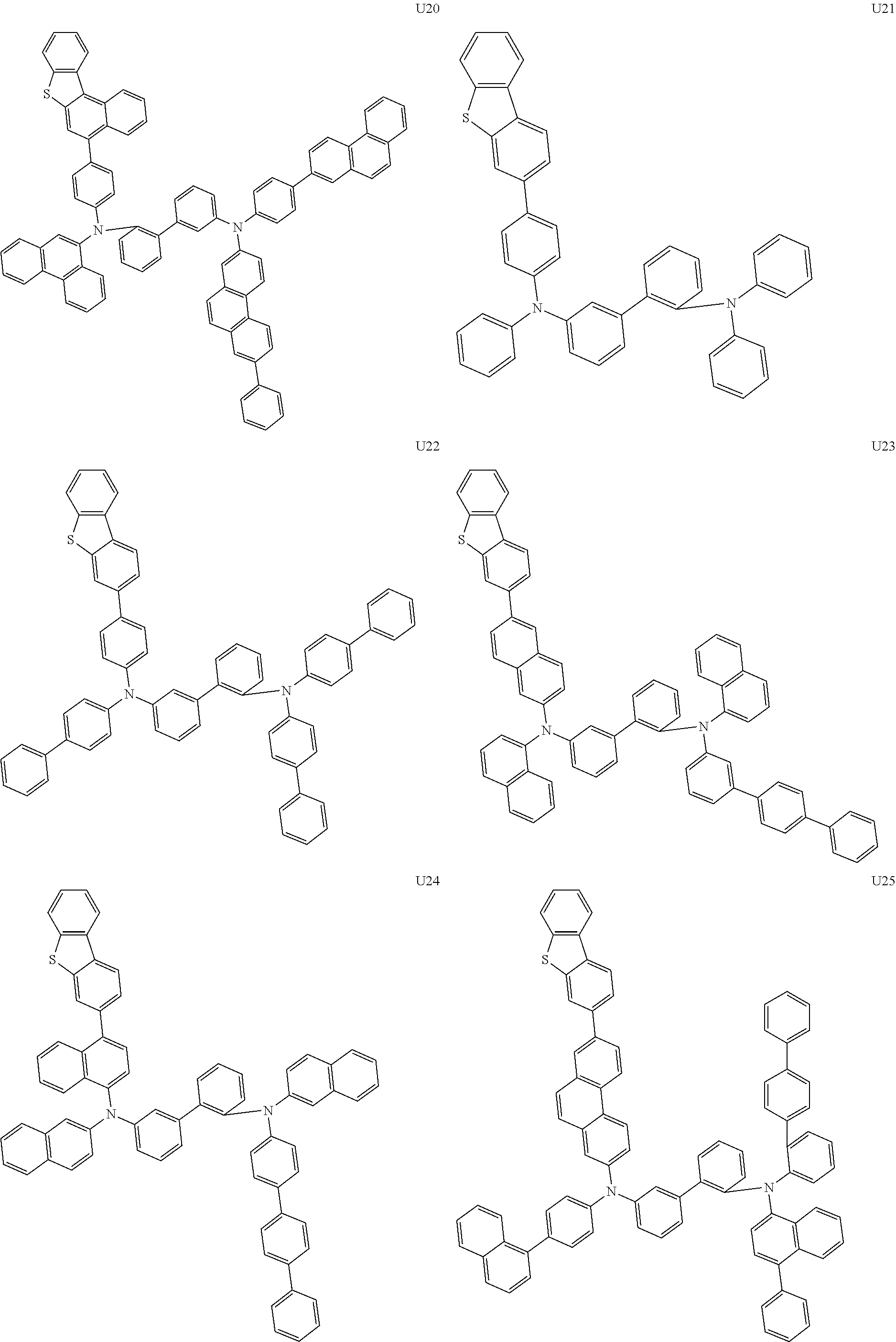 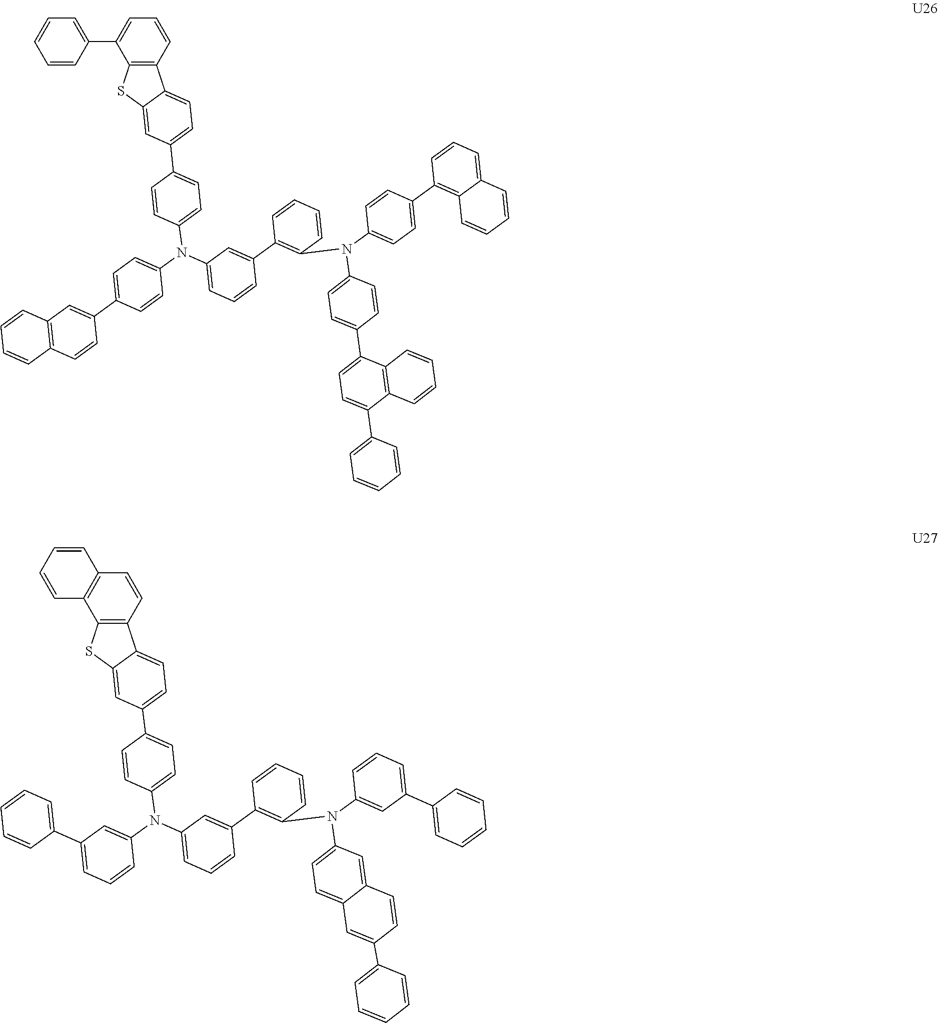 [SMILES: c1ccc(-c2ccc(-c3ccc(N(c4ccc(-c5cccc6ccccc56)cc4)c4ccccc4-c4cccc(N(c5ccc(-c6ccc7ccccc7c6)cc5)c5ccc(-c6ccc7c(c6)sc6c(-c8ccccc8)cccc67)cc5)c4)cc3)c3ccccc23)cc1 ; c1ccc(-c2cccc(N(c3ccc(-c4ccc5c(c4)sc4c6ccccc6ccc54)cc3)c3cccc(-c4ccccc4N(c4cccc(-c5ccccc5)c4)c4ccc5cc(-c6ccccc6)ccc5c4)c3)c2)cc1] 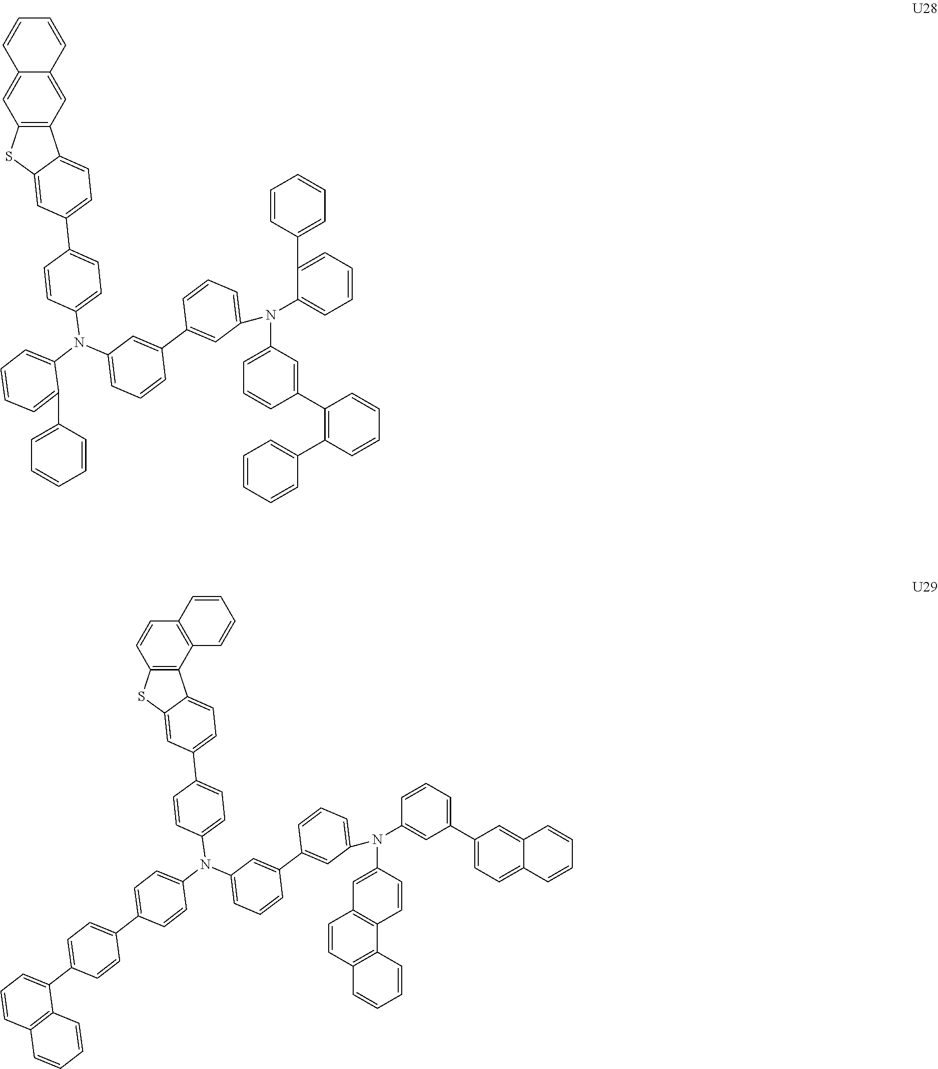 [SMILES: c1ccc(-c2ccccc2-c2cccc(N(c3cccc(-c4cccc(N(c5ccc(-c6ccc7c(c6)sc6cc8ccccc8cc67)cc5)c5ccccc5-c5ccccc5)c4)c3)c3ccccc3-c3ccccc3)c2)cc1 ; c1cc(-c2cccc(N(c3cccc(-c4ccc5ccccc5c4)c3)c3ccc4c(ccc5ccccc54)c3)c2)cc(N(c2ccc(-c3ccc(-c4cccc5ccccc45)cc3)cc2)c2ccc(-c3ccc4c(c3)sc3ccc5ccccc5c34)cc2)c1] 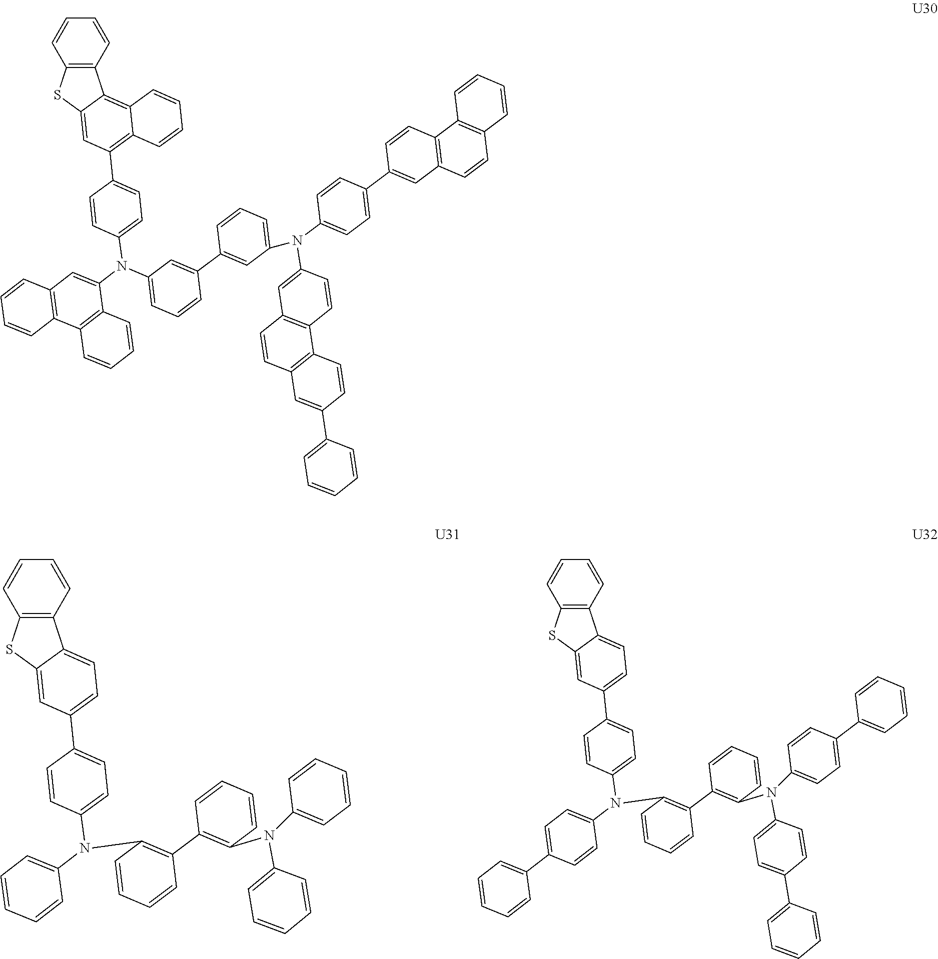 [SMILES: c1ccc(-c2ccc3c(ccc4cc(N(c5ccc(-c6ccc7c(ccc8ccccc87)c6)cc5)c5cccc(-c6cccc(N(c7ccc(-c8cc9sc%10ccccc%10c9c9ccccc89)cc7)c7cc8ccccc8c8ccccc78)c6)c5)ccc43)c2)cc1 ; c1ccc(N(c2ccccc2)c2ccccc2-c2ccccc2N(c2ccccc2)c2ccc(-c3ccc4c(c3)sc3ccccc34)cc2)cc1 ; c1ccc(-c2ccc(N(c3ccc(-c4ccccc4)cc3)c3ccccc3-c3ccccc3N(c3ccc(-c4ccccc4)cc3)c3ccc(-c4ccc5c(c4)sc4ccccc45)cc3)cc2)cc1] 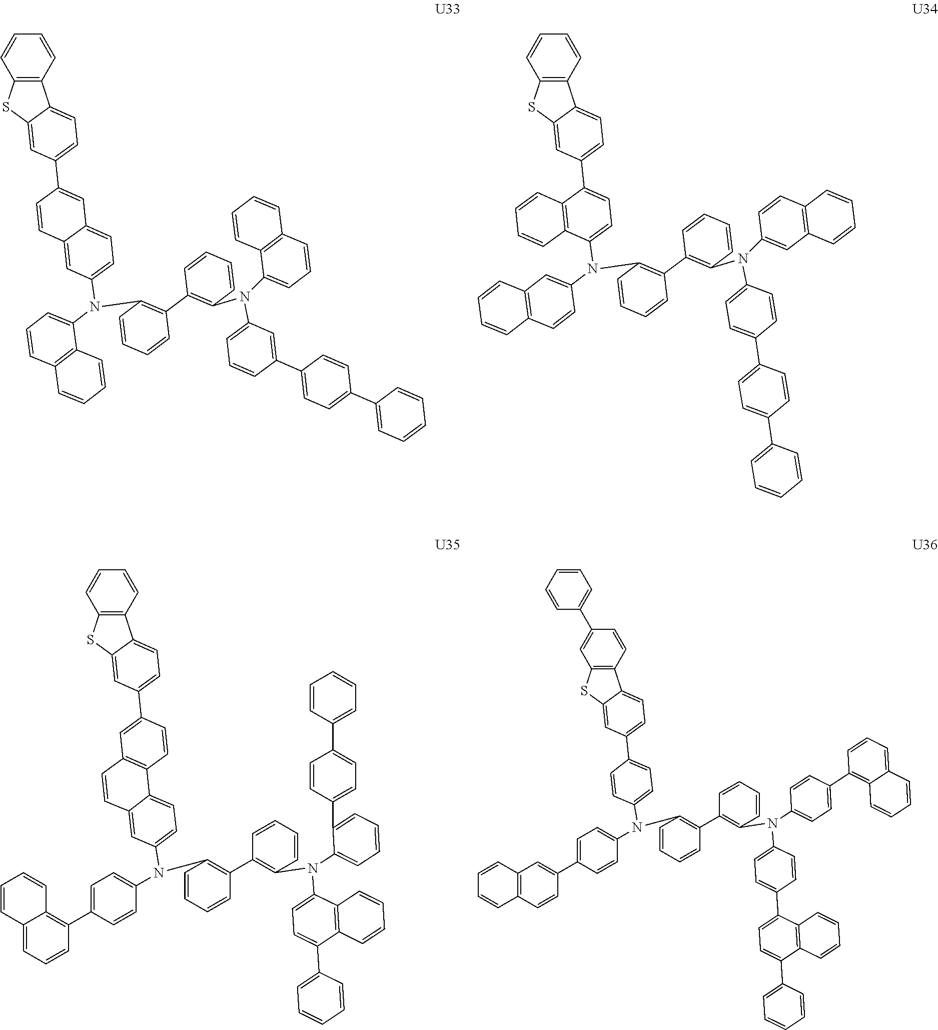 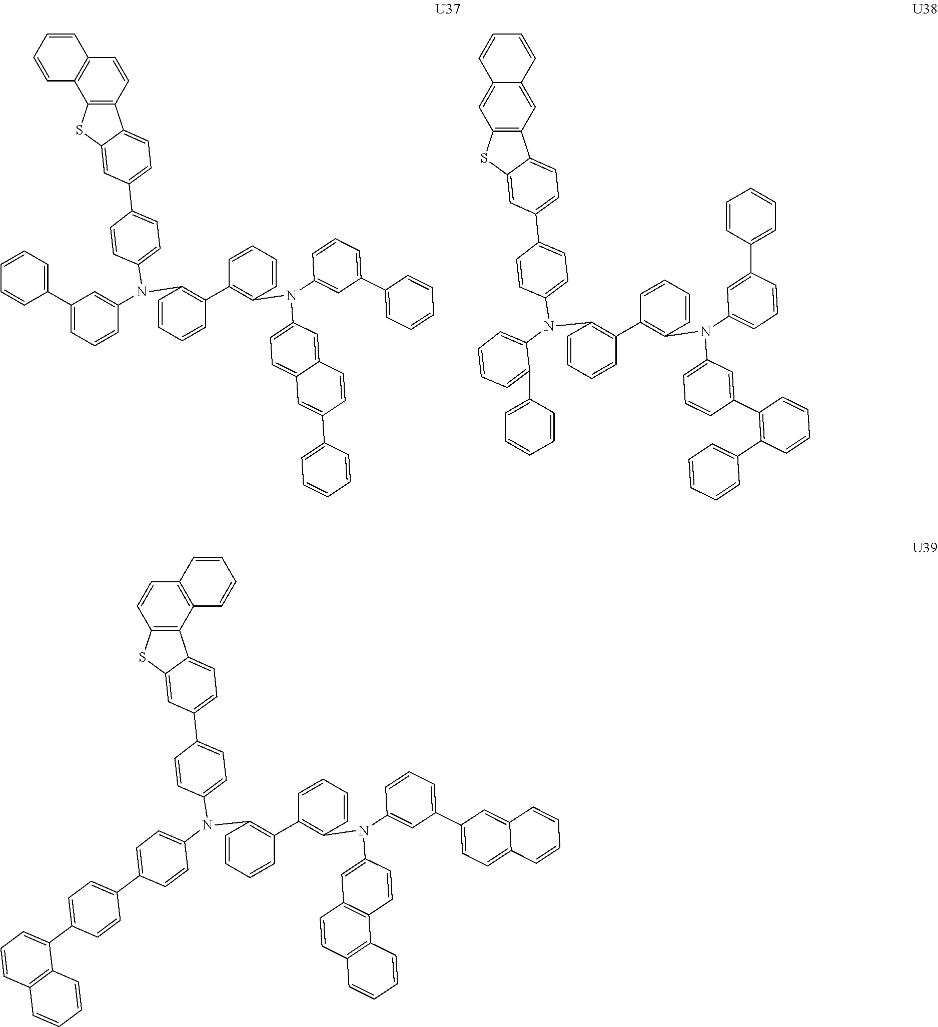 [SMILES: c1ccc(-c2cccc(N(c3ccc(-c4ccc5c(c4)sc4c6ccccc6ccc54)cc3)c3ccccc3-c3ccccc3N(c3cccc(-c4ccccc4)c3)c3ccc4cc(-c5ccccc5)ccc4c3)c2)cc1 ; c1ccc(-c2cccc(N(c3cccc(-c4ccccc4-c4ccccc4)c3)c3ccccc3-c3ccccc3N(c3ccc(-c4ccc5c(c4)sc4cc6ccccc6cc45)cc3)c3ccccc3-c3ccccc3)c2)cc1 ; c1cc(-c2ccc3ccccc3c2)cc(N(c2ccc3c(ccc4ccccc43)c2)c2ccccc2-c2ccccc2N(c2ccc(-c3ccc(-c4cccc5ccccc45)cc3)cc2)c2ccc(-c3ccc4c(c3)sc3ccc5ccccc5c34)cc2)c1] 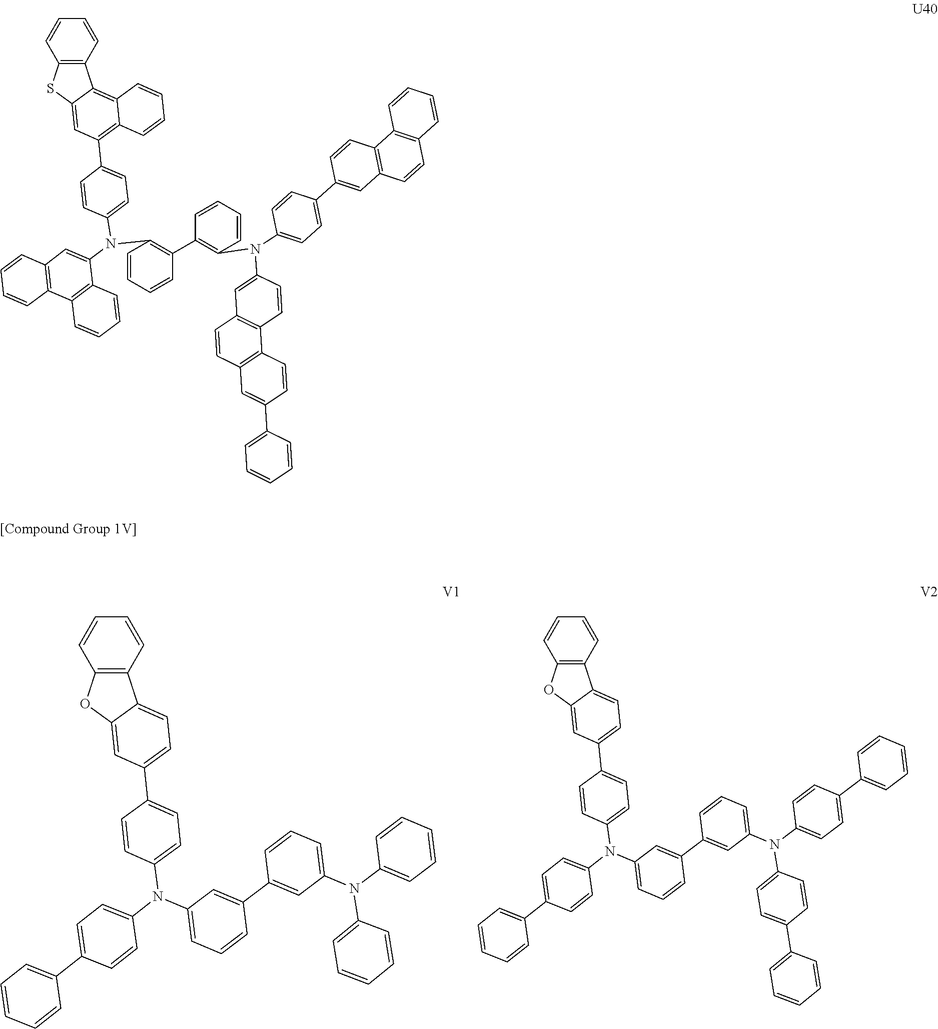 [SMILES: c1ccc(-c2ccc3c(ccc4cc(N(c5ccc(-c6ccc7c(ccc8ccccc87)c6)cc5)c5ccccc5-c5ccccc5N(c5ccc(-c6cc7sc8ccccc8c7c7ccccc67)cc5)c5cc6ccccc6c6ccccc56)ccc43)c2)cc1 ; c1ccc(-c2ccc(N(c3ccc(-c4ccc5c(c4)oc4ccccc45)cc3)c3cccc(-c4cccc(N(c5ccccc5)c5ccccc5)c4)c3)cc2)cc1 ; c1ccc(-c2ccc(N(c3ccc(-c4ccccc4)cc3)c3cccc(-c4cccc(N(c5ccc(-c6ccccc6)cc5)c5ccc(-c6ccc7c(c6)oc6ccccc67)cc5)c4)c3)cc2)cc1] 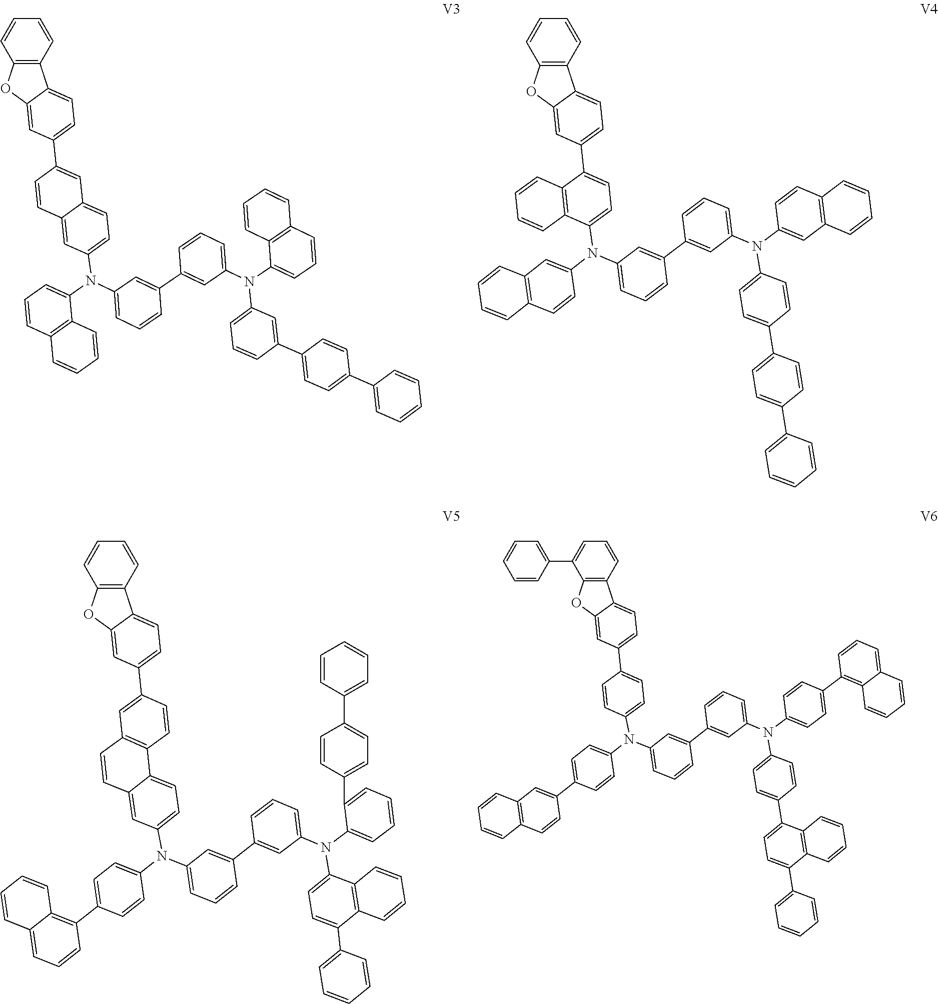 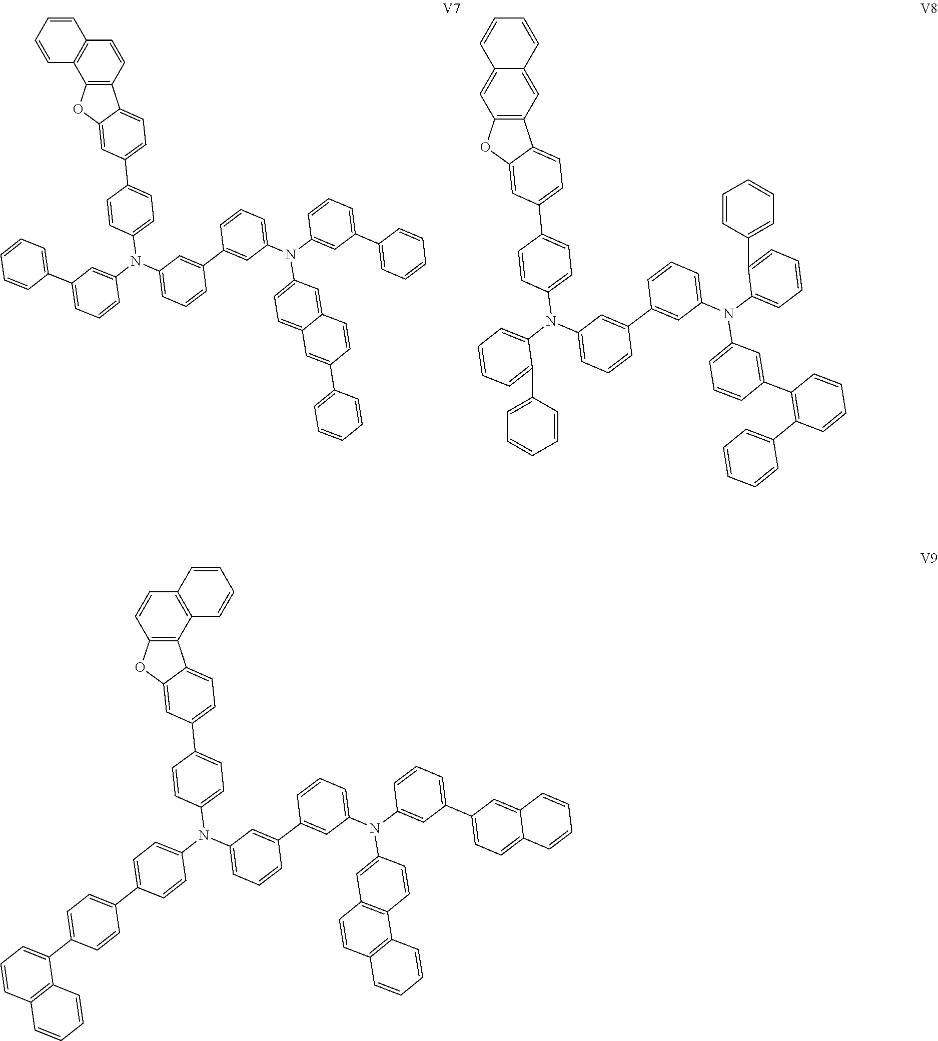 [SMILES: c1ccc(-c2cccc(N(c3ccc(-c4ccc5c(c4)oc4c6ccccc6ccc54)cc3)c3cccc(-c4cccc(N(c5cccc(-c6ccccc6)c5)c5ccc6cc(-c7ccccc7)ccc6c5)c4)c3)c2)cc1 ; c1ccc(-c2ccccc2-c2cccc(N(c3cccc(-c4cccc(N(c5ccc(-c6ccc7c(c6)oc6cc8ccccc8cc67)cc5)c5ccccc5-c5ccccc5)c4)c3)c3ccccc3-c3ccccc3)c2)cc1 ; c1cc(-c2cccc(N(c3cccc(-c4ccc5ccccc5c4)c3)c3ccc4c(ccc5ccccc54)c3)c2)cc(N(c2ccc(-c3ccc(-c4cccc5ccccc45)cc3)cc2)c2ccc(-c3ccc4c(c3)oc3ccc5ccccc5c34)cc2)c1] 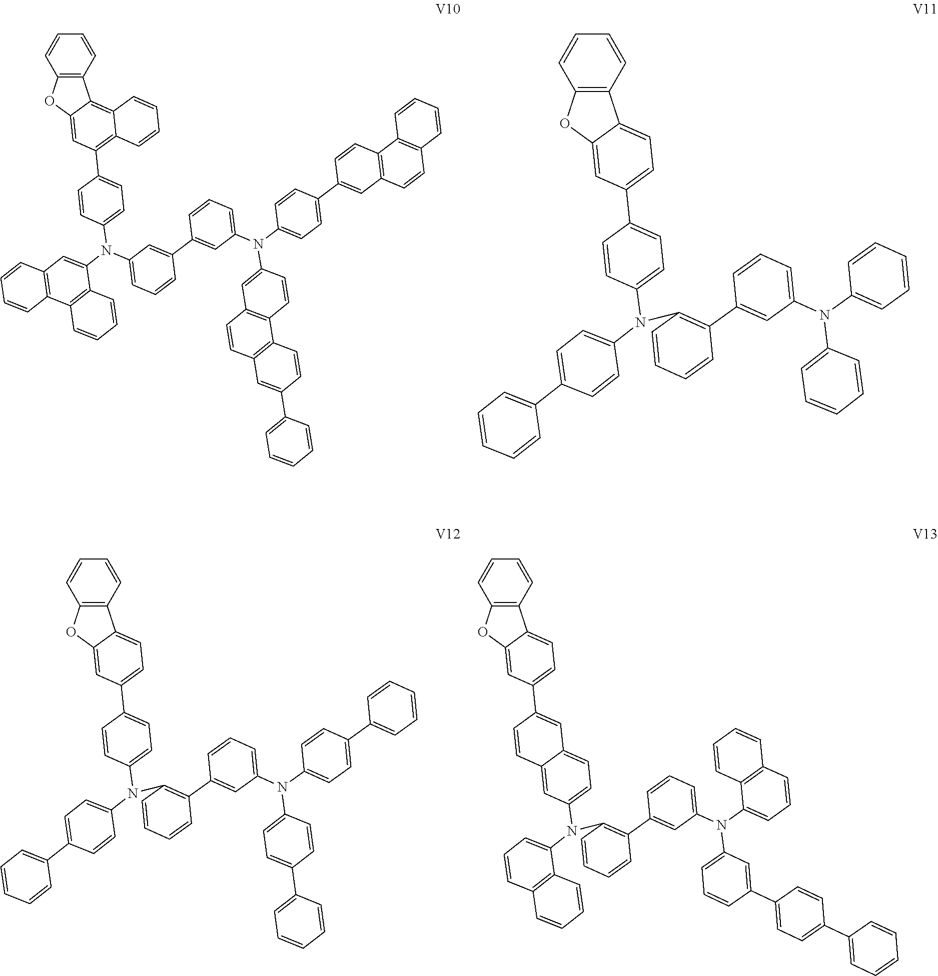 [SMILES: c1ccc(-c2ccc3c(ccc4cc(N(c5ccc(-c6ccc7c(ccc8ccccc87)c6)cc5)c5cccc(-c6cccc(N(c7ccc(-c8cc9oc%10ccccc%10c9c9ccccc89)cc7)c7cc8ccccc8c8ccccc78)c6)c5)ccc43)c2)cc1 ; c1ccc(-c2ccc(N(c3ccc(-c4ccc5c(c4)oc4ccccc45)cc3)c3ccccc3-c3cccc(N(c4ccccc4)c4ccccc4)c3)cc2)cc1 ; c1ccc(-c2ccc(N(c3ccc(-c4ccccc4)cc3)c3cccc(-c4ccccc4N(c4ccc(-c5ccccc5)cc4)c4ccc(-c5ccc6c(c5)oc5ccccc56)cc4)c3)cc2)cc1 ; c1ccc(-c2ccc(-c3cccc(N(c4cccc(-c5ccccc5N(c5ccc6cc(-c7ccc8c(c7)oc7ccccc78)ccc6c5)c5cccc6ccccc56)c4)c4cccc5ccccc45)c3)cc2)cc1] 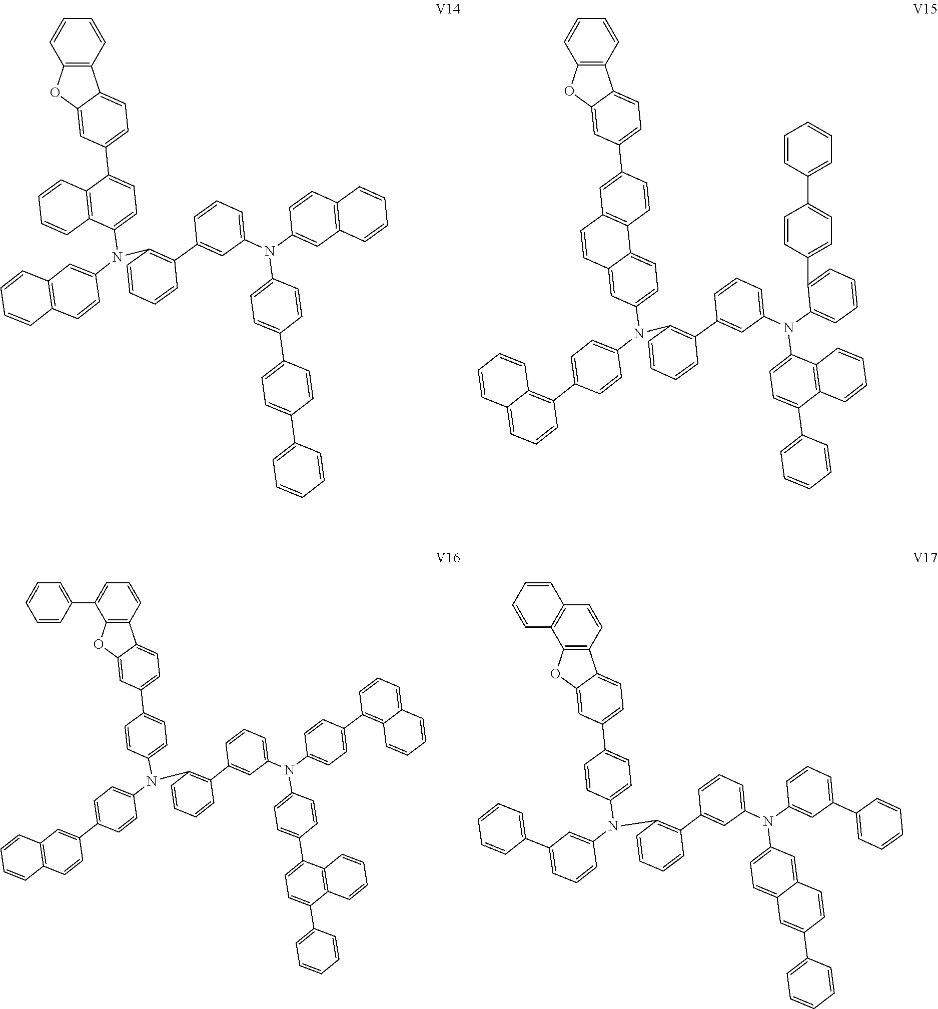 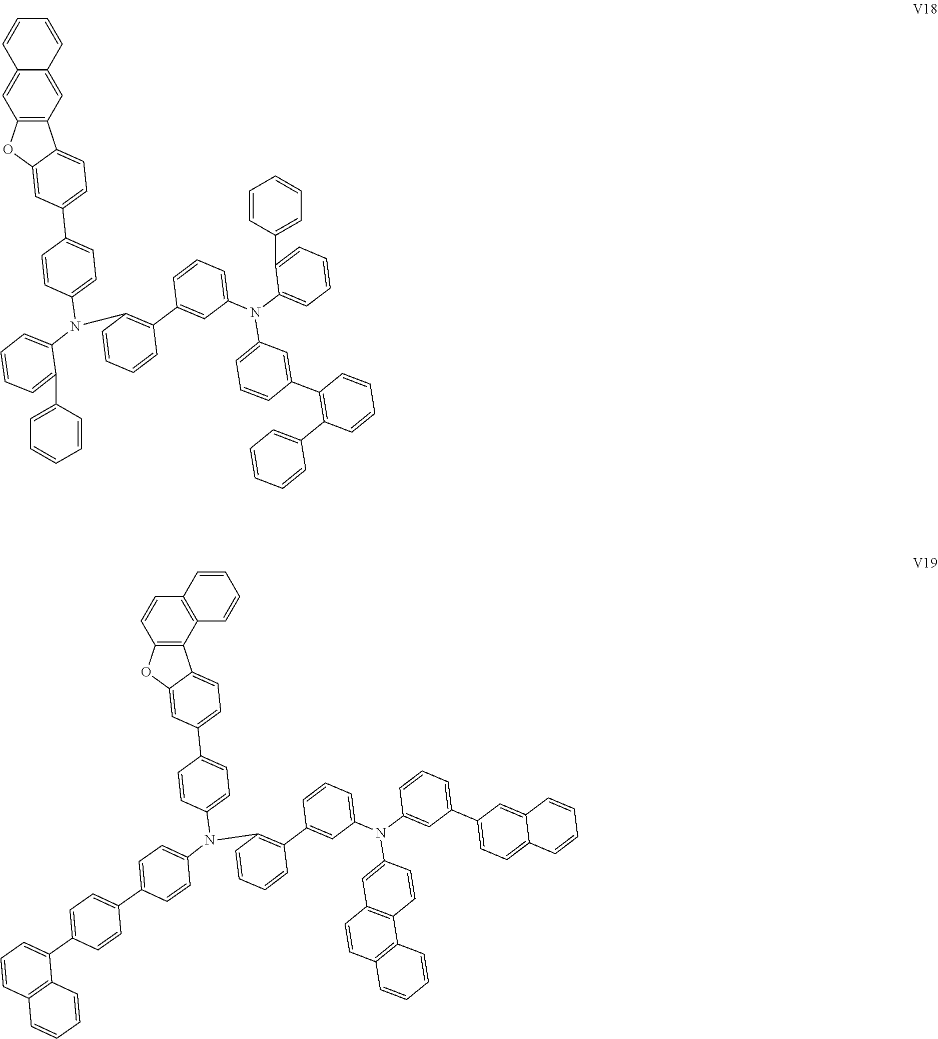 [SMILES: c1ccc(-c2ccccc2-c2cccc(N(c3cccc(-c4ccccc4N(c4ccc(-c5ccc6c(c5)oc5cc7ccccc7cc56)cc4)c4ccccc4-c4ccccc4)c3)c3ccccc3-c3ccccc3)c2)cc1 ; c1cc(-c2ccc3ccccc3c2)cc(N(c2cccc(-c3ccccc3N(c3ccc(-c4ccc(-c5cccc6ccccc56)cc4)cc3)c3ccc(-c4ccc5c(c4)oc4ccc6ccccc6c45)cc3)c2)c2ccc3c(ccc4ccccc43)c2)c1] 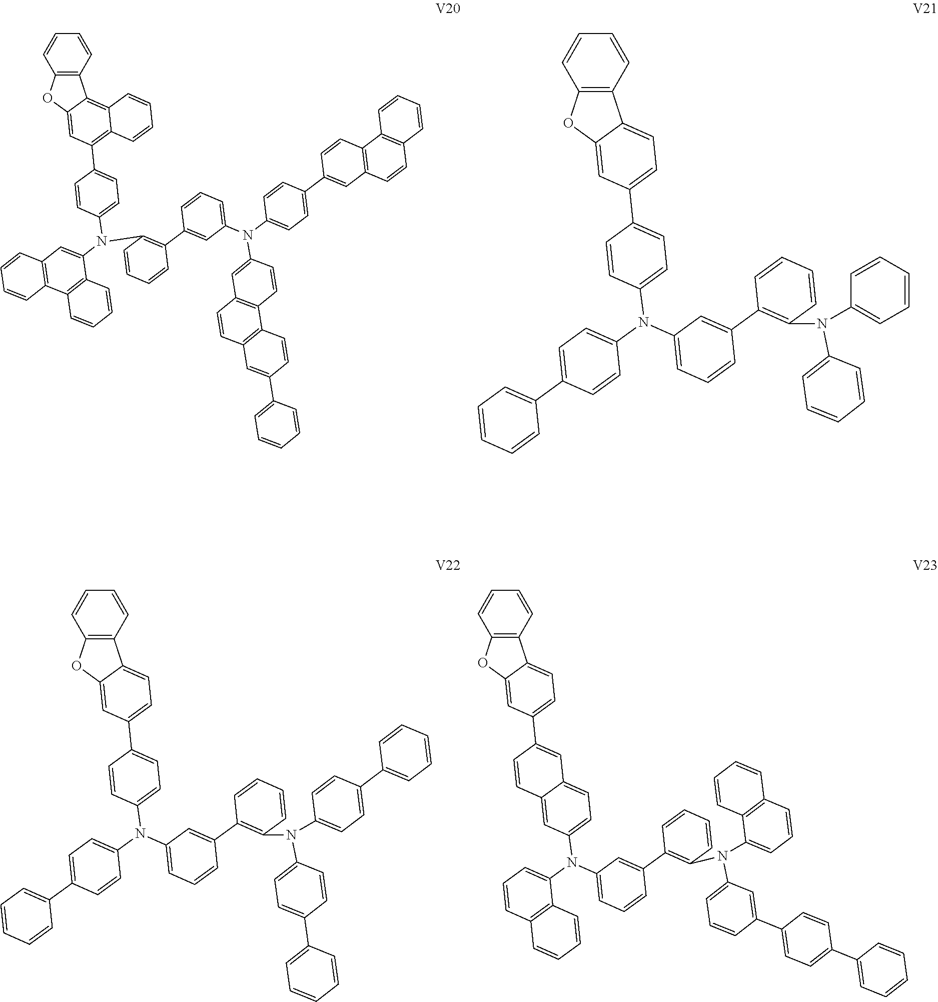 [SMILES: c1ccc(-c2ccc3c(ccc4cc(N(c5ccc(-c6ccc7c(ccc8ccccc87)c6)cc5)c5cccc(-c6ccccc6N(c6ccc(-c7cc8oc9ccccc9c8c8ccccc78)cc6)c6cc7ccccc7c7ccccc67)c5)ccc43)c2)cc1 ; c1ccc(-c2ccc(N(c3ccc(-c4ccc5c(c4)oc4ccccc45)cc3)c3cccc(-c4ccccc4N(c4ccccc4)c4ccccc4)c3)cc2)cc1 ; c1ccc(-c2ccc(N(c3ccc(-c4ccc5c(c4)oc4ccccc45)cc3)c3cccc(-c4ccccc4N(c4ccc(-c5ccccc5)cc4)c4ccc(-c5ccccc5)cc4)c3)cc2)cc1 ; c1ccc(-c2ccc(-c3cccc(N(c4ccccc4-c4cccc(N(c5ccc6cc(-c7ccc8c(c7)oc7ccccc78)ccc6c5)c5cccc6ccccc56)c4)c4cccc5ccccc45)c3)cc2)cc1] 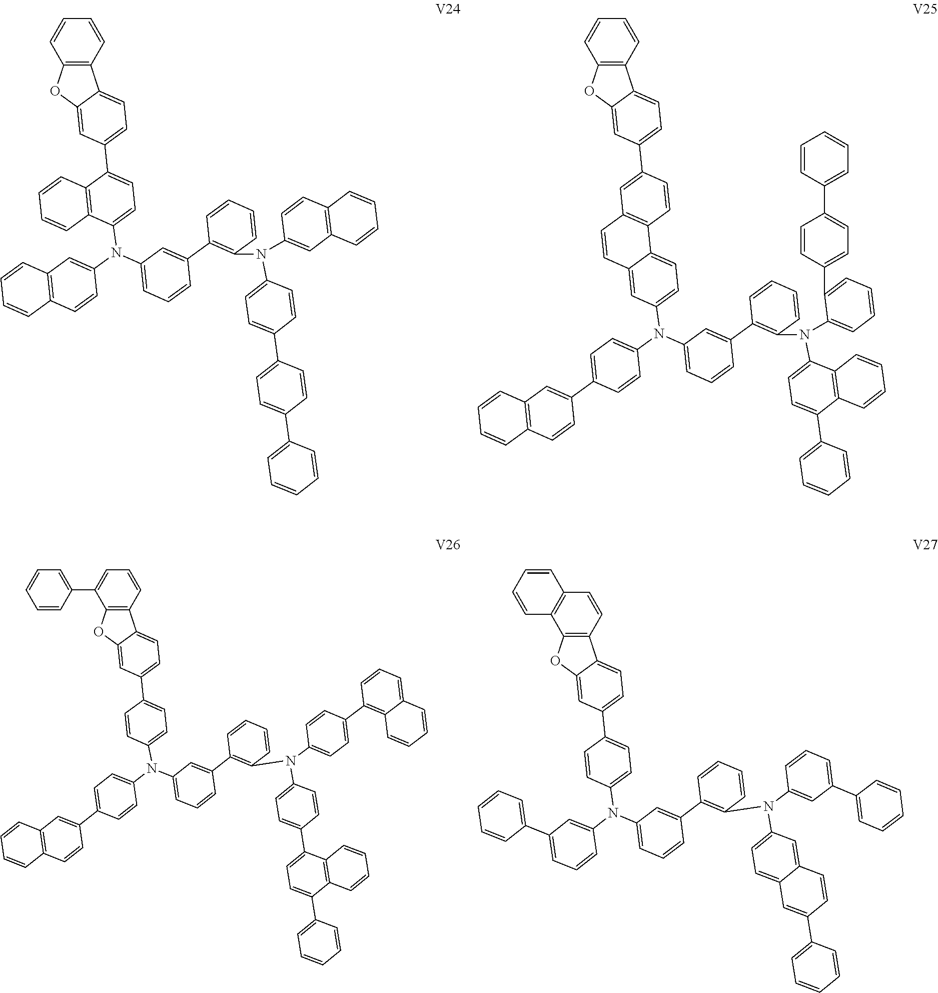 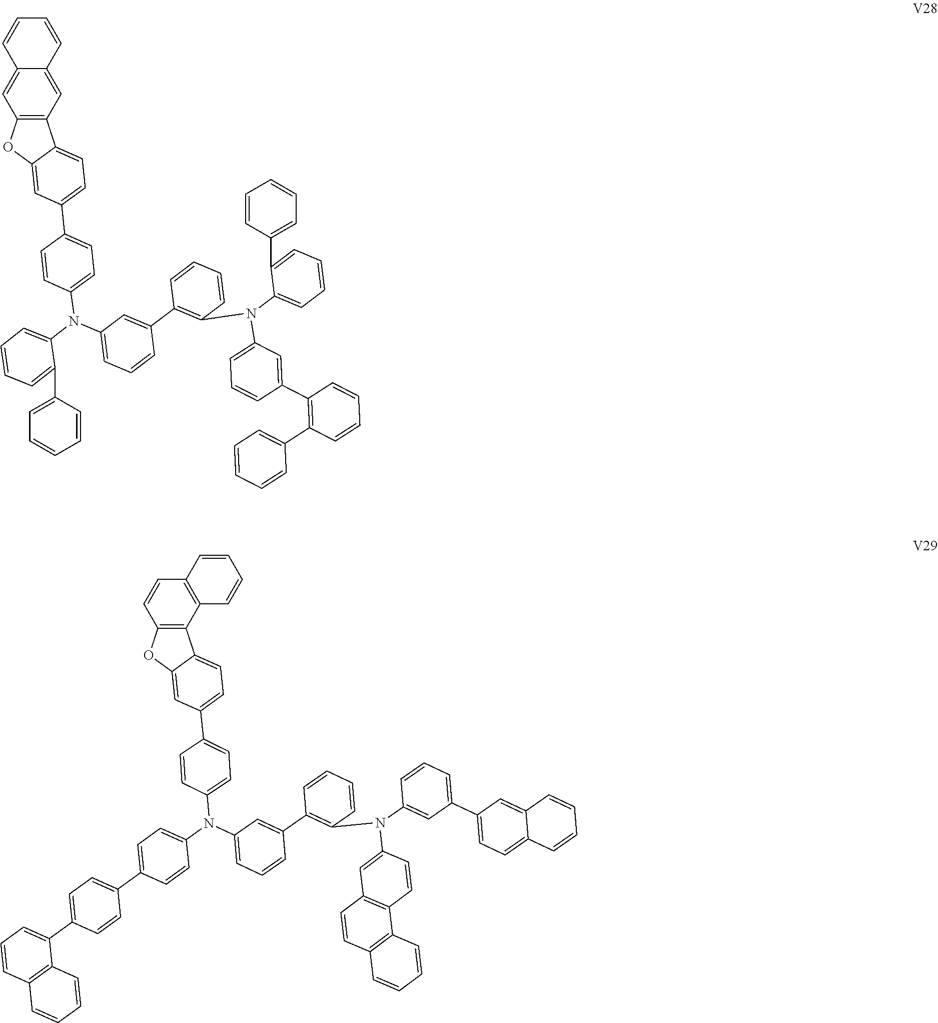 [SMILES: c1ccc(-c2ccccc2-c2cccc(N(c3ccccc3-c3ccccc3)c3ccccc3-c3cccc(N(c4ccc(-c5ccc6c(c5)oc5cc7ccccc7cc56)cc4)c4ccccc4-c4ccccc4)c3)c2)cc1 ; c1cc(-c2ccccc2N(c2cccc(-c3ccc4ccccc4c3)c2)c2ccc3c(ccc4ccccc43)c2)cc(N(c2ccc(-c3ccc(-c4cccc5ccccc45)cc3)cc2)c2ccc(-c3ccc4c(c3)oc3ccc5ccccc5c34)cc2)c1] 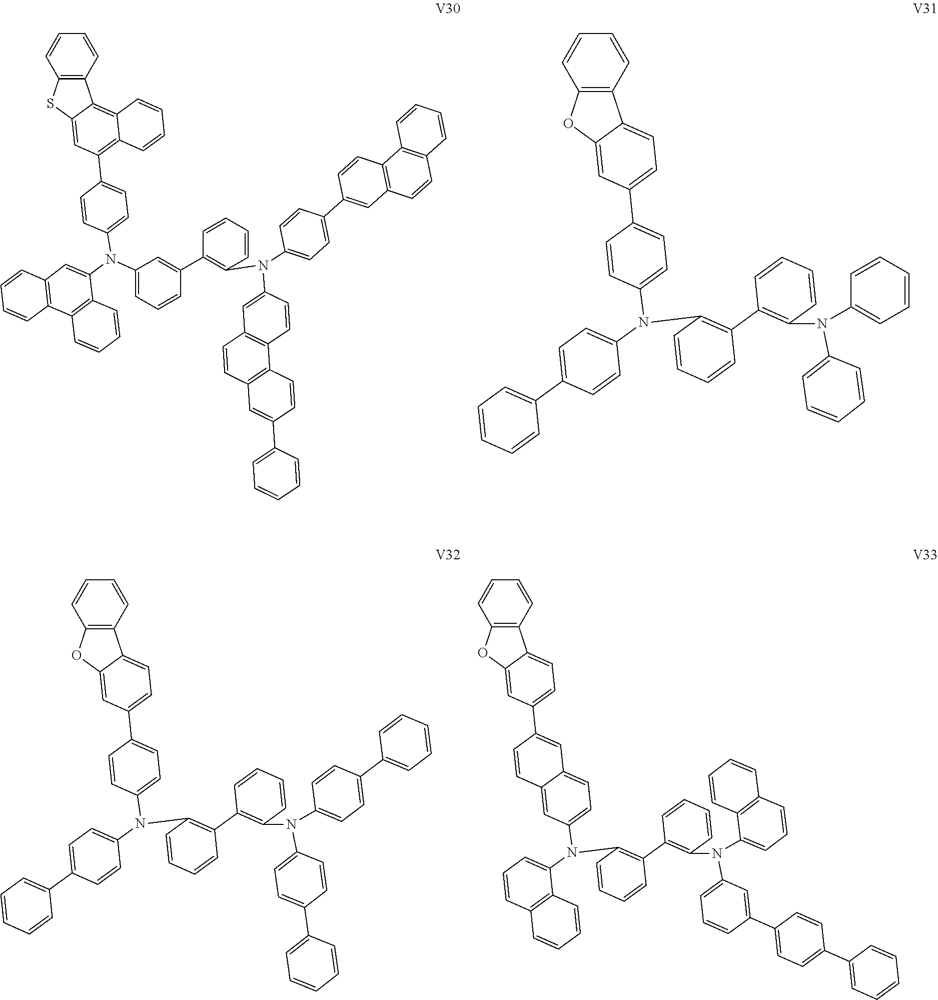 [SMILES: c1ccc(-c2ccc3c(ccc4cc(N(c5ccc(-c6ccc7c(ccc8ccccc87)c6)cc5)c5ccccc5-c5cccc(N(c6ccc(-c7cc8sc9ccccc9c8c8ccccc78)cc6)c6cc7ccccc7c7ccccc67)c5)ccc43)c2)cc1 ; c1ccc(-c2ccc(N(c3ccc(-c4ccc5c(c4)oc4ccccc45)cc3)c3ccccc3-c3ccccc3N(c3ccccc3)c3ccccc3)cc2)cc1 ; c1ccc(-c2ccc(N(c3ccc(-c4ccccc4)cc3)c3ccccc3-c3ccccc3N(c3ccc(-c4ccccc4)cc3)c3ccc(-c4ccc5c(c4)oc4ccccc45)cc3)cc2)cc1 ; c1ccc(-c2ccc(-c3cccc(N(c4ccccc4-c4ccccc4N(c4ccc5cc(-c6ccc7c(c6)oc6ccccc67)ccc5c4)c4cccc5ccccc45)c4cccc5ccccc45)c3)cc2)cc1] 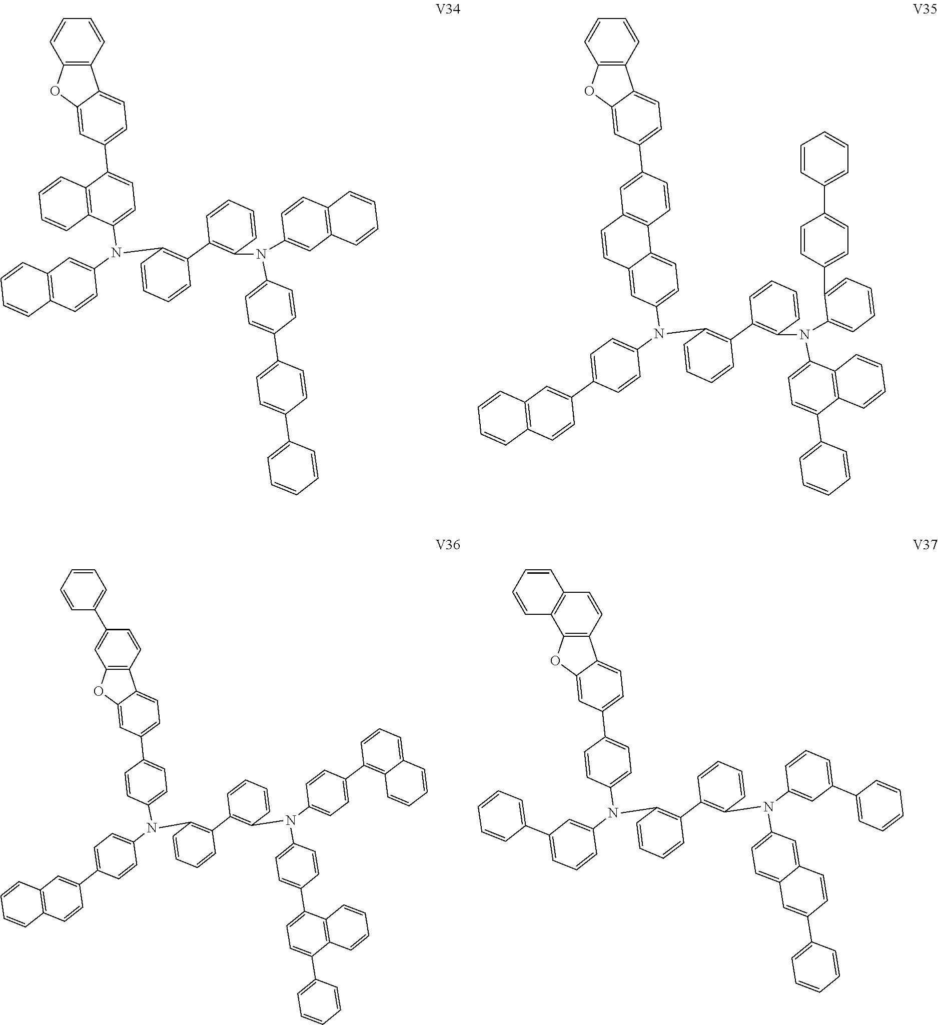 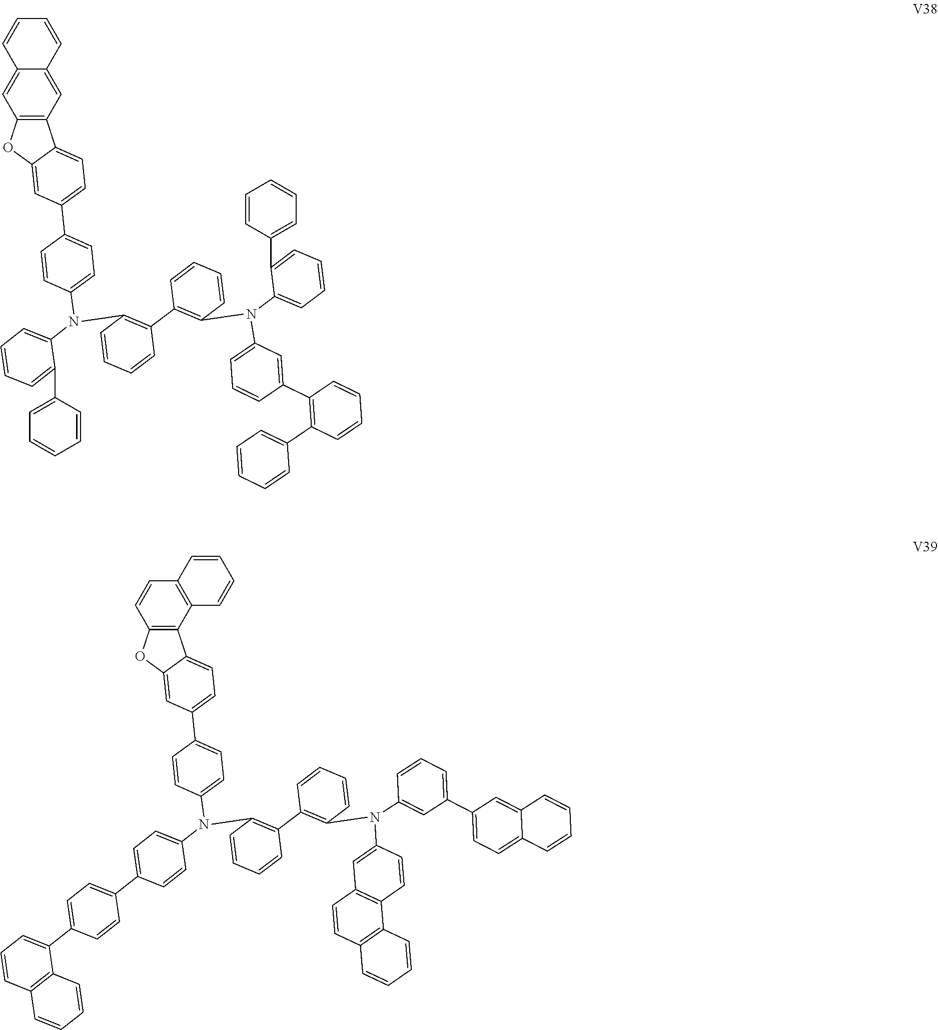 [SMILES: c1ccc(-c2ccccc2-c2cccc(N(c3ccccc3-c3ccccc3)c3ccccc3-c3ccccc3N(c3ccc(-c4ccc5c(c4)oc4cc6ccccc6cc45)cc3)c3ccccc3-c3ccccc3)c2)cc1 ; c1cc(-c2ccc3ccccc3c2)cc(N(c2ccc3c(ccc4ccccc43)c2)c2ccccc2-c2ccccc2N(c2ccc(-c3ccc(-c4cccc5ccccc45)cc3)cc2)c2ccc(-c3ccc4c(c3)oc3ccc5ccccc5c34)cc2)c1] 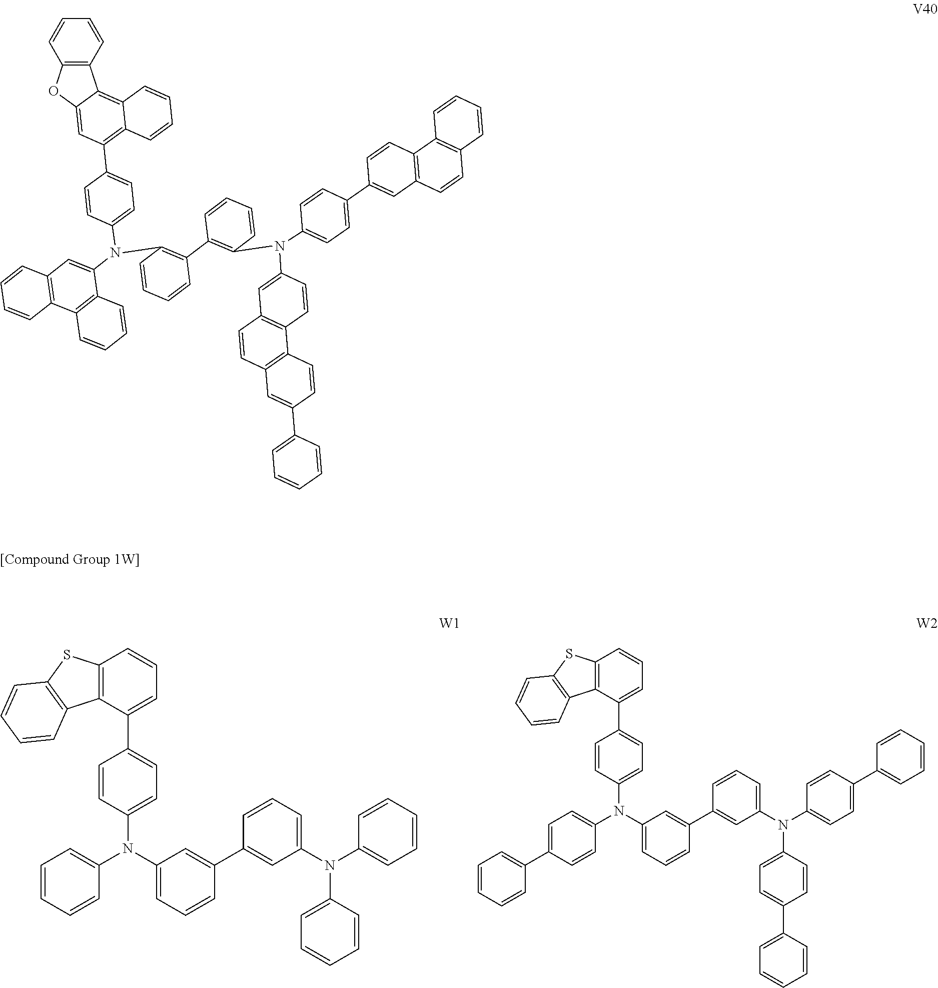 [SMILES: c1ccc(-c2ccc3c(ccc4cc(N(c5ccc(-c6ccc7c(ccc8ccccc87)c6)cc5)c5ccccc5-c5ccccc5N(c5ccc(-c6cc7oc8ccccc8c7c7ccccc67)cc5)c5cc6ccccc6c6ccccc56)ccc43)c2)cc1 ; c1ccc(N(c2ccccc2)c2cccc(-c3cccc(N(c4ccccc4)c4ccc(-c5cccc6sc7ccccc7c56)cc4)c3)c2)cc1 ; c1ccc(-c2ccc(N(c3ccc(-c4ccccc4)cc3)c3cccc(-c4cccc(N(c5ccc(-c6ccccc6)cc5)c5ccc(-c6cccc7sc8ccccc8c67)cc5)c4)c3)cc2)cc1] 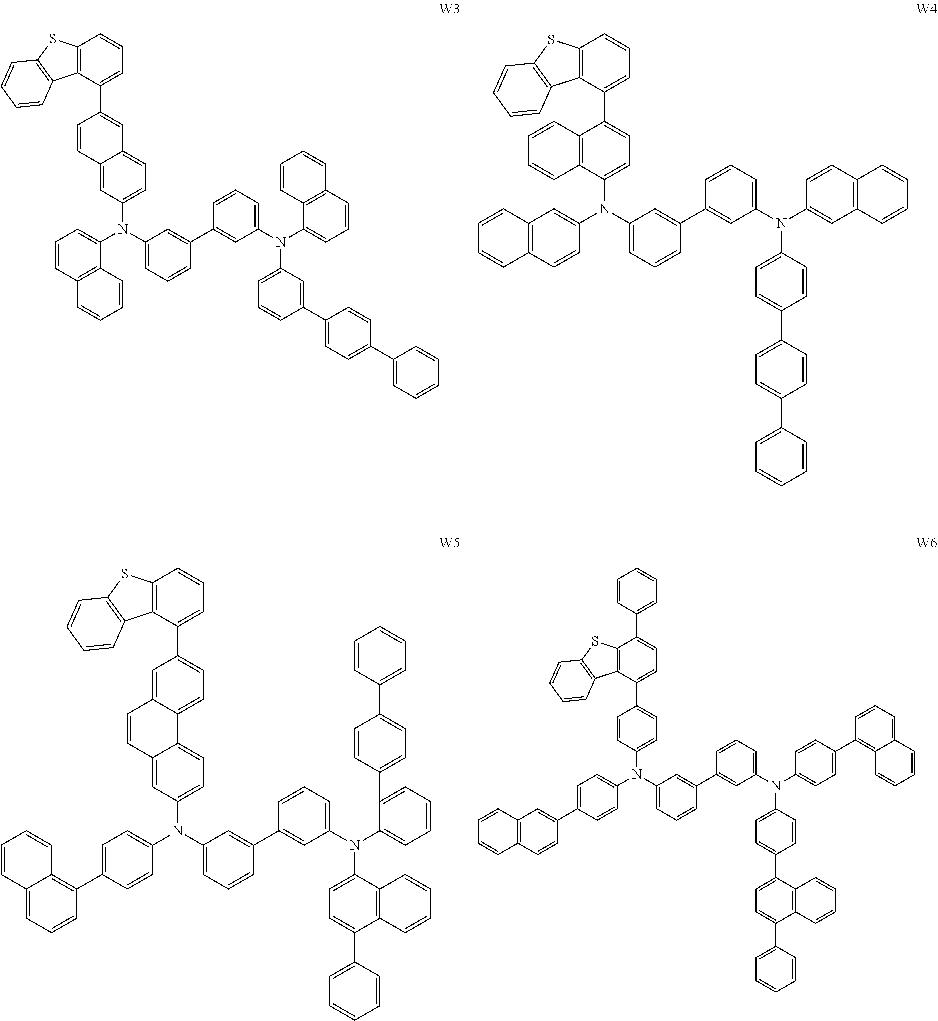 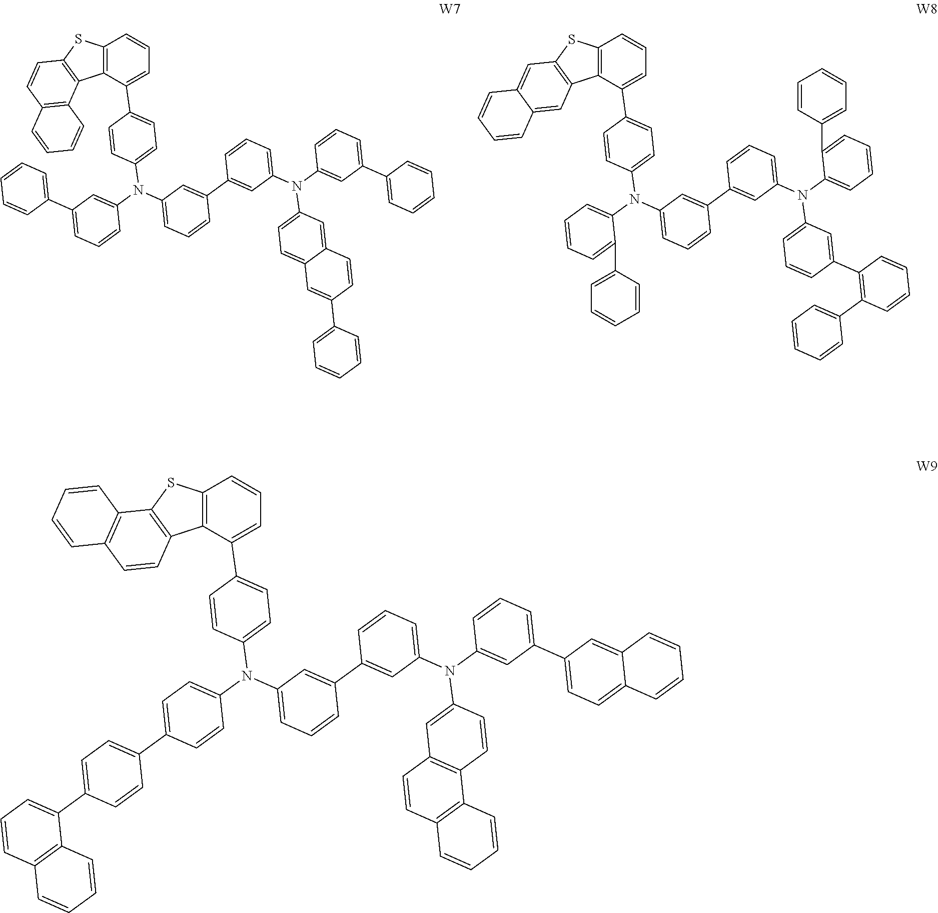 [SMILES: c1ccc(-c2cccc(N(c3ccc(-c4cccc5sc6ccc7ccccc7c6c45)cc3)c3cccc(-c4cccc(N(c5cccc(-c6ccccc6)c5)c5ccc6cc(-c7ccccc7)ccc6c5)c4)c3)c2)cc1 ; c1ccc(-c2ccccc2-c2cccc(N(c3cccc(-c4cccc(N(c5ccc(-c6cccc7sc8cc9ccccc9cc8c67)cc5)c5ccccc5-c5ccccc5)c4)c3)c3ccccc3-c3ccccc3)c2)cc1 ; c1cc(-c2cccc(N(c3cccc(-c4ccc5ccccc5c4)c3)c3ccc4c(ccc5ccccc54)c3)c2)cc(N(c2ccc(-c3ccc(-c4cccc5ccccc45)cc3)cc2)c2ccc(-c3cccc4sc5c6ccccc6ccc5c34)cc2)c1] 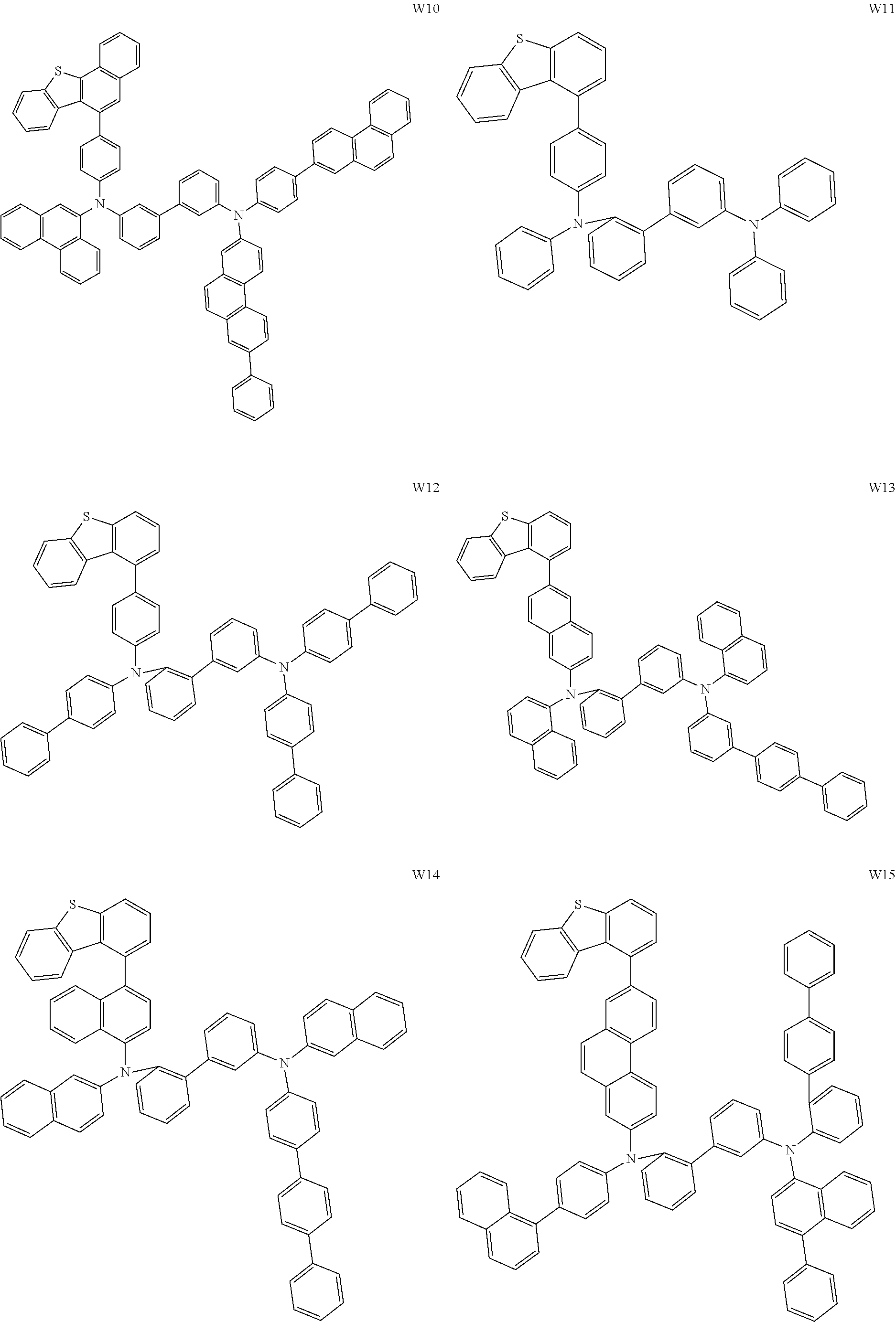 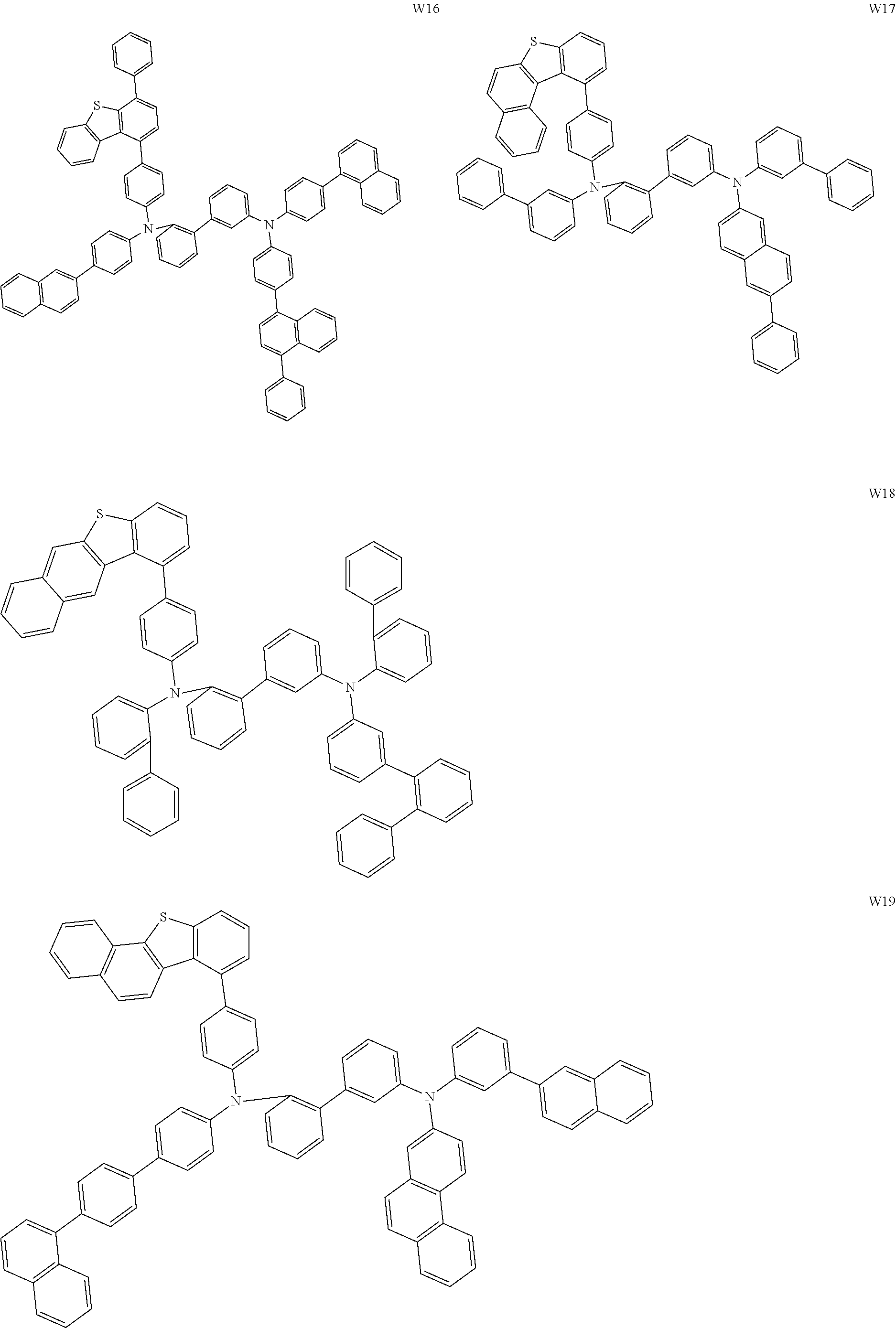 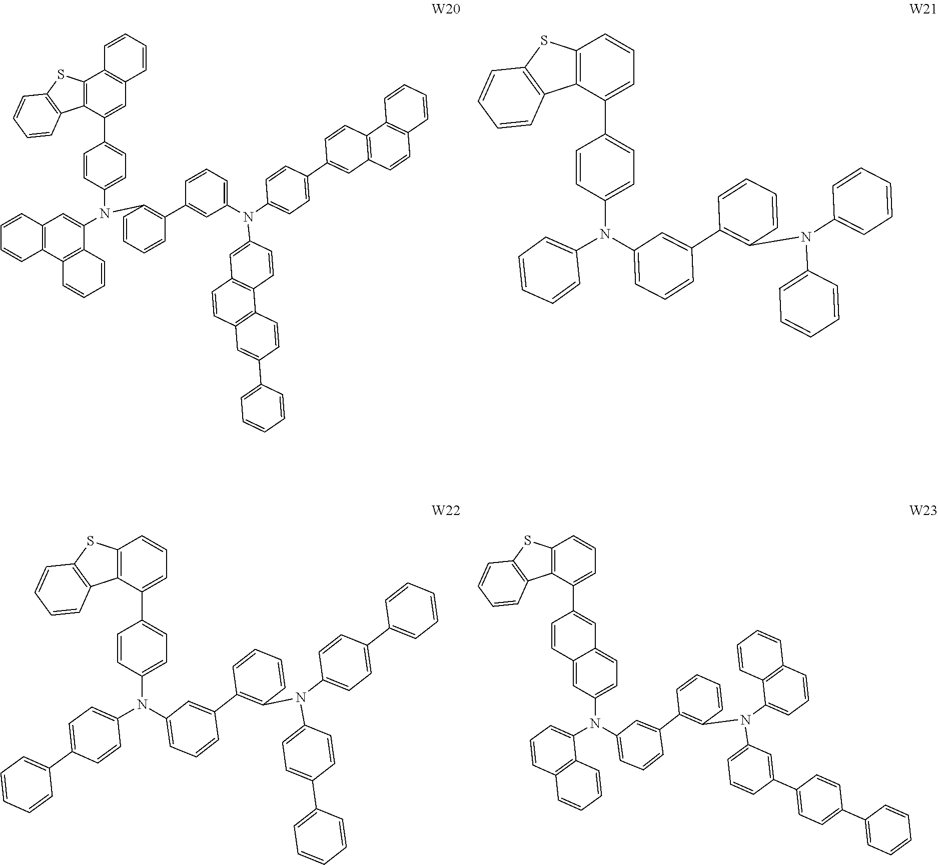 [SMILES: c1ccc(-c2ccc3c(ccc4cc(N(c5ccc(-c6ccc7c(ccc8ccccc87)c6)cc5)c5cccc(-c6ccccc6N(c6ccc(-c7cc8ccccc8c8sc9ccccc9c78)cc6)c6cc7ccccc7c7ccccc67)c5)ccc43)c2)cc1 ; c1ccc(N(c2ccc(-c3cccc4sc5ccccc5c34)cc2)c2cccc(-c3ccccc3N(c3ccccc3)c3ccccc3)c2)cc1 ; c1ccc(-c2ccc(N(c3ccc(-c4cccc5sc6ccccc6c45)cc3)c3cccc(-c4ccccc4N(c4ccc(-c5ccccc5)cc4)c4ccc(-c5ccccc5)cc4)c3)cc2)cc1 ; c1ccc(-c2ccc(-c3cccc(N(c4ccccc4-c4cccc(N(c5ccc6cc(-c7cccc8sc9ccccc9c78)ccc6c5)c5cccc6ccccc56)c4)c4cccc5ccccc45)c3)cc2)cc1] 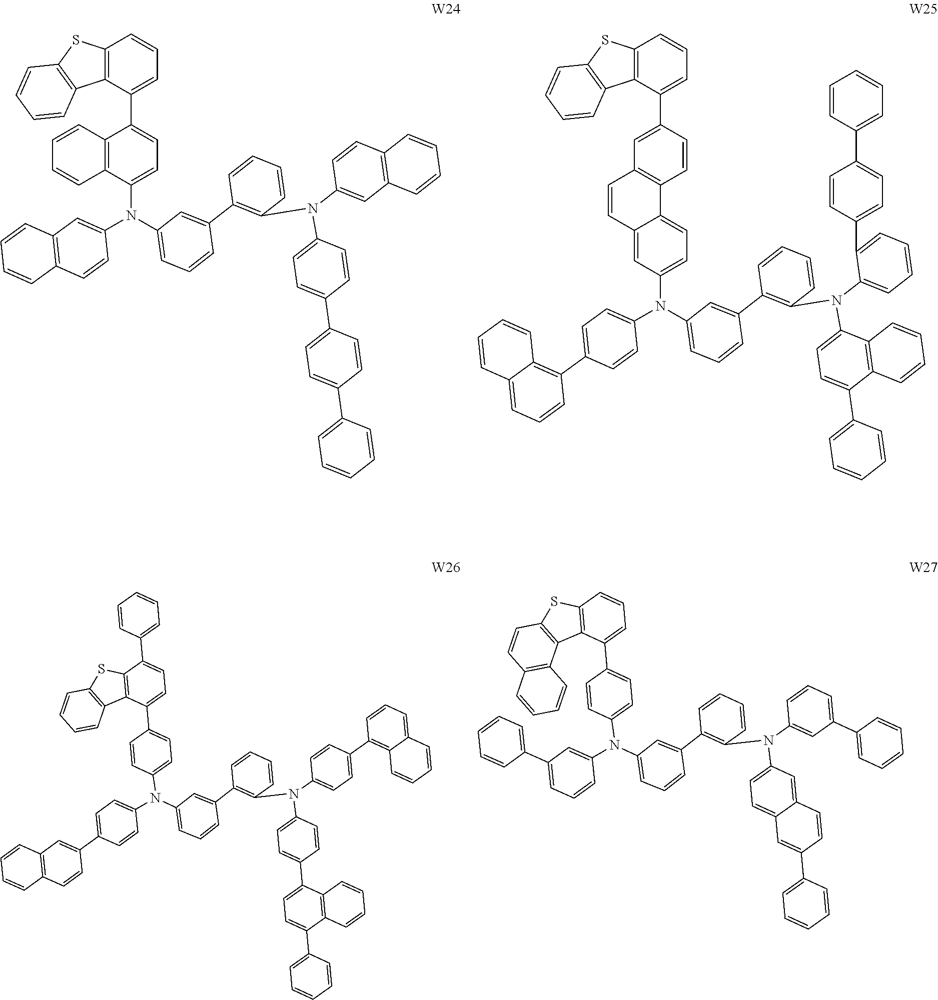 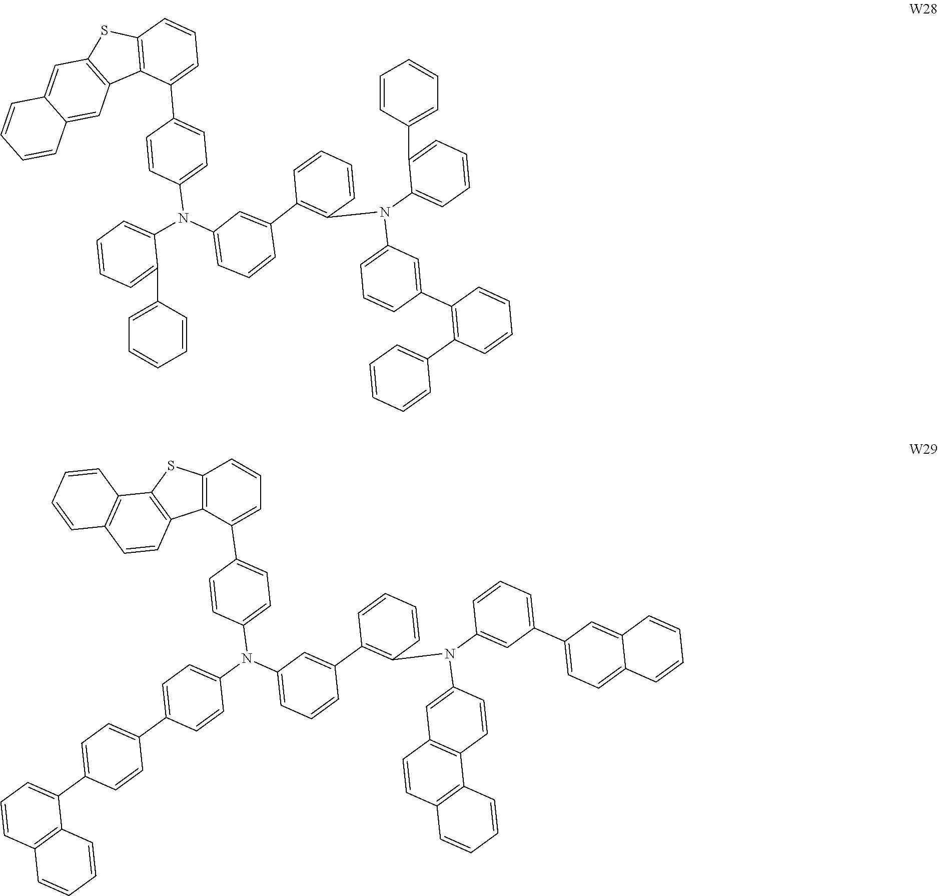 [SMILES: c1ccc(-c2ccccc2-c2cccc(N(c3ccccc3-c3ccccc3)c3ccccc3-c3cccc(N(c4ccc(-c5cccc6sc7cc8ccccc8cc7c56)cc4)c4ccccc4-c4ccccc4)c3)c2)cc1 ; c1cc(-c2ccccc2N(c2cccc(-c3ccc4ccccc4c3)c2)c2ccc3c(ccc4ccccc43)c2)cc(N(c2ccc(-c3ccc(-c4cccc5ccccc45)cc3)cc2)c2ccc(-c3cccc4sc5c6ccccc6ccc5c34)cc2)c1] 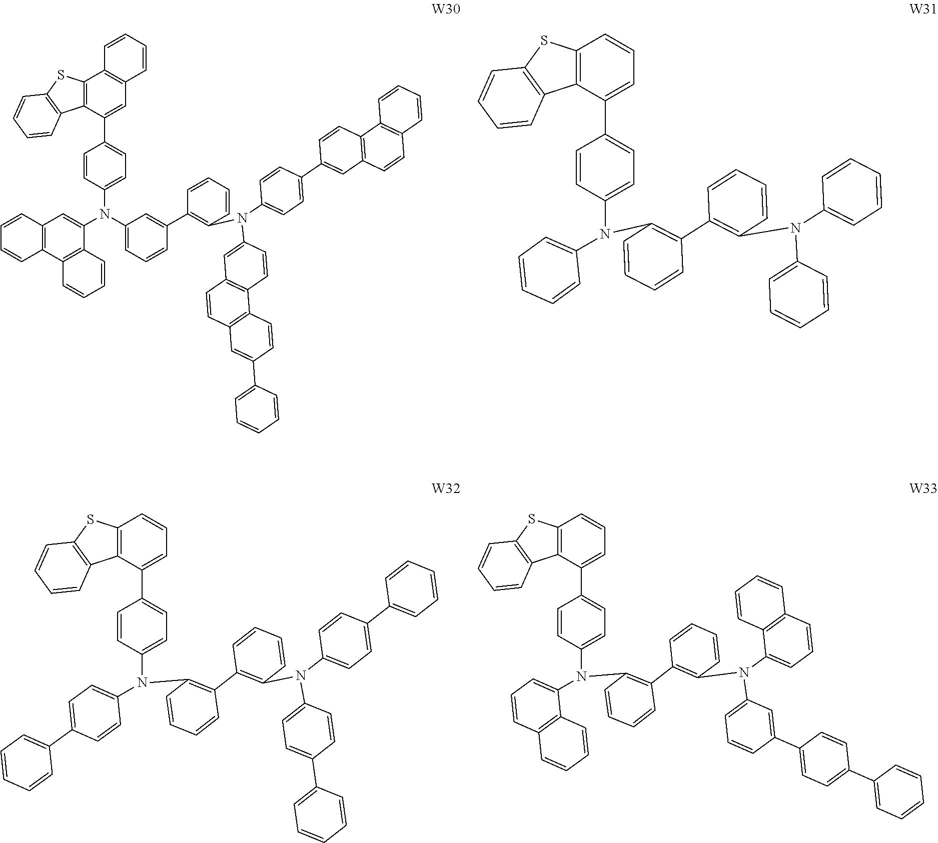 [SMILES: c1ccc(-c2ccc3c(ccc4cc(N(c5ccc(-c6ccc7c(ccc8ccccc87)c6)cc5)c5ccccc5-c5cccc(N(c6ccc(-c7cc8ccccc8c8sc9ccccc9c78)cc6)c6cc7ccccc7c7ccccc67)c5)ccc43)c2)cc1 ; c1ccc(N(c2ccccc2)c2ccccc2-c2ccccc2N(c2ccccc2)c2ccc(-c3cccc4sc5ccccc5c34)cc2)cc1 ; c1ccc(-c2ccc(N(c3ccc(-c4ccccc4)cc3)c3ccccc3-c3ccccc3N(c3ccc(-c4ccccc4)cc3)c3ccc(-c4cccc5sc6ccccc6c45)cc3)cc2)cc1 ; c1ccc(-c2ccc(-c3cccc(N(c4ccccc4-c4ccccc4N(c4ccc(-c5cccc6sc7ccccc7c56)cc4)c4cccc5ccccc45)c4cccc5ccccc45)c3)cc2)cc1] 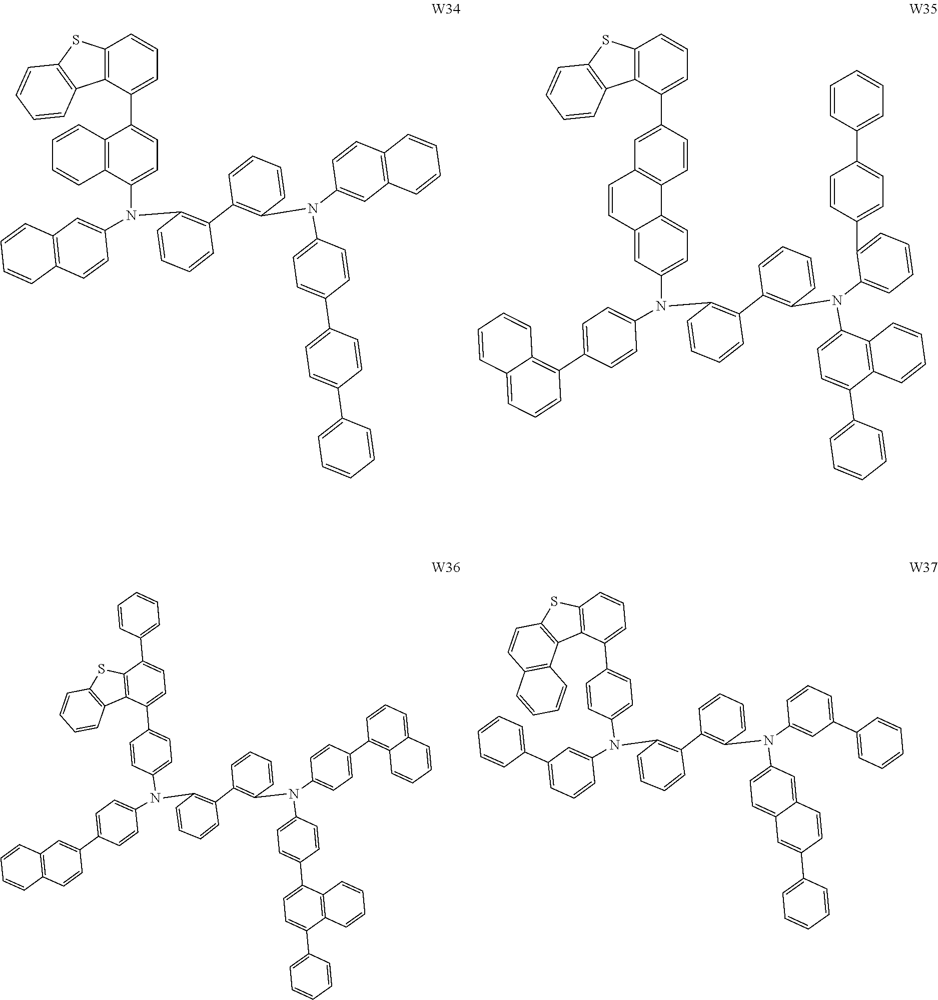 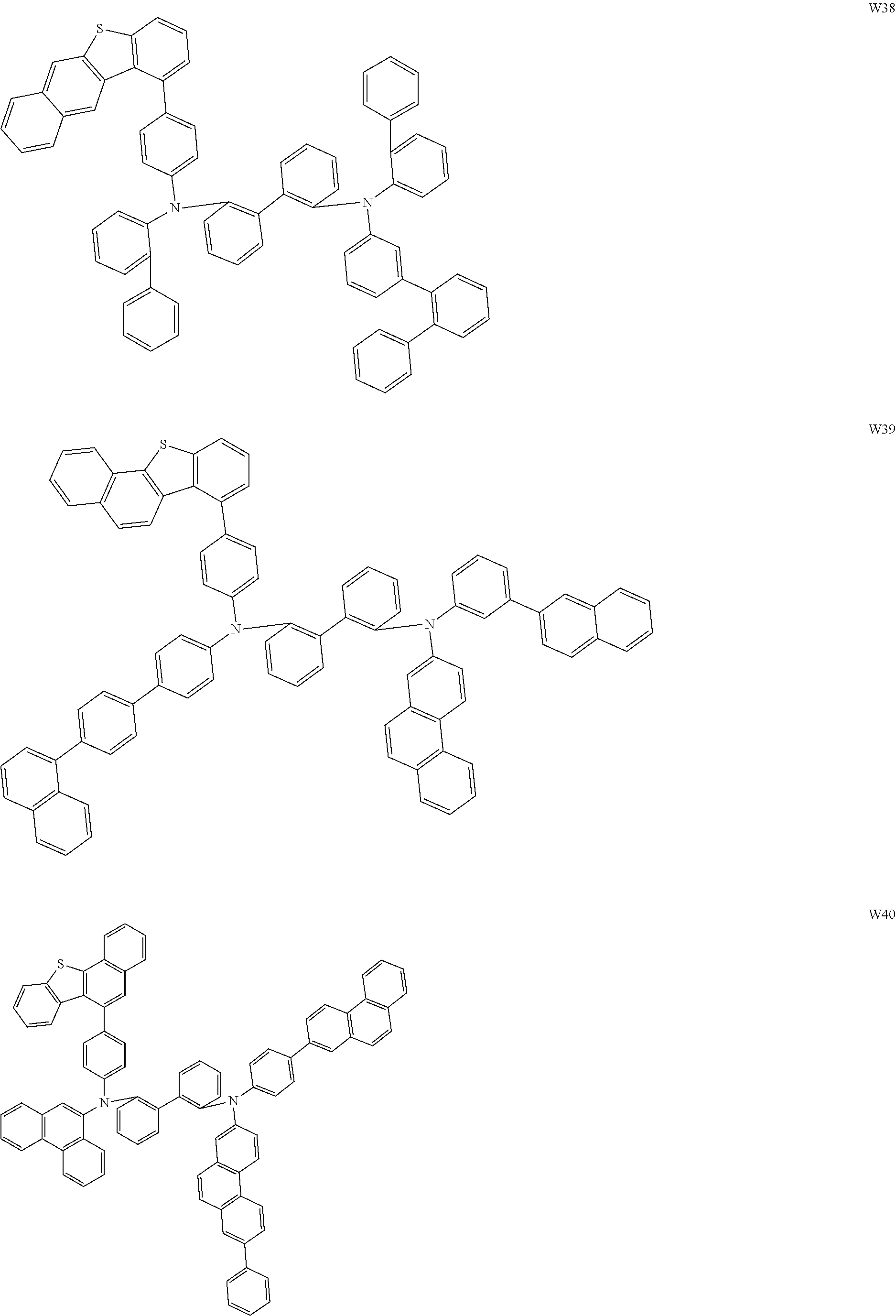 [SMILES: c1ccc(-c2ccccc2-c2cccc(N(c3ccccc3-c3ccccc3)c3ccccc3-c3ccccc3N(c3ccc(-c4cccc5sc6cc7ccccc7cc6c45)cc3)c3ccccc3-c3ccccc3)c2)cc1 ; c1cc(-c2ccc3ccccc3c2)cc(N(c2ccc3c(ccc4ccccc43)c2)c2ccccc2-c2ccccc2N(c2ccc(-c3ccc(-c4cccc5ccccc45)cc3)cc2)c2ccc(-c3cccc4sc5c6ccccc6ccc5c34)cc2)c1 ; c1ccc(-c2ccc3c(ccc4cc(N(c5ccc(-c6ccc7c(ccc8ccccc87)c6)cc5)c5ccccc5-c5ccccc5N(c5ccc(-c6cc7ccccc7c7sc8ccccc8c67)cc5)c5cc6ccccc6c6ccccc56)ccc43)c2)cc1] 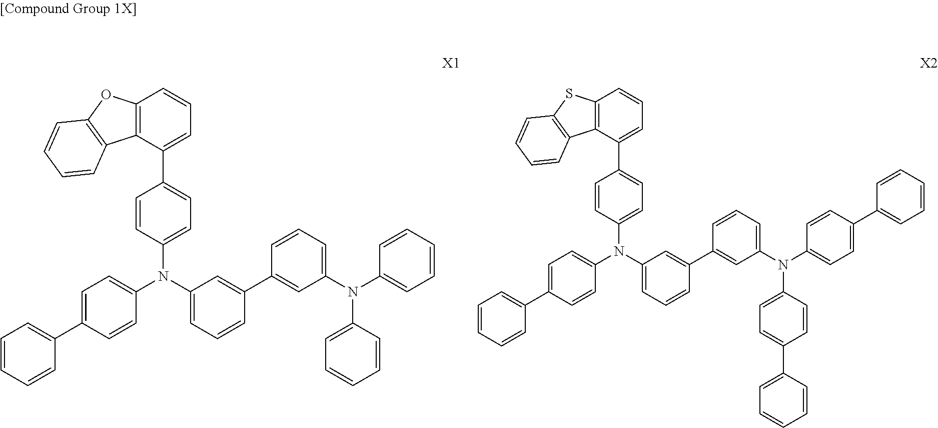 [SMILES: c1ccc(-c2ccc(N(c3ccc(-c4cccc5oc6ccccc6c45)cc3)c3cccc(-c4cccc(N(c5ccccc5)c5ccccc5)c4)c3)cc2)cc1 ; c1ccc(-c2ccc(N(c3ccc(-c4ccccc4)cc3)c3cccc(-c4cccc(N(c5ccc(-c6ccccc6)cc5)c5ccc(-c6cccc7sc8ccccc8c67)cc5)c4)c3)cc2)cc1] 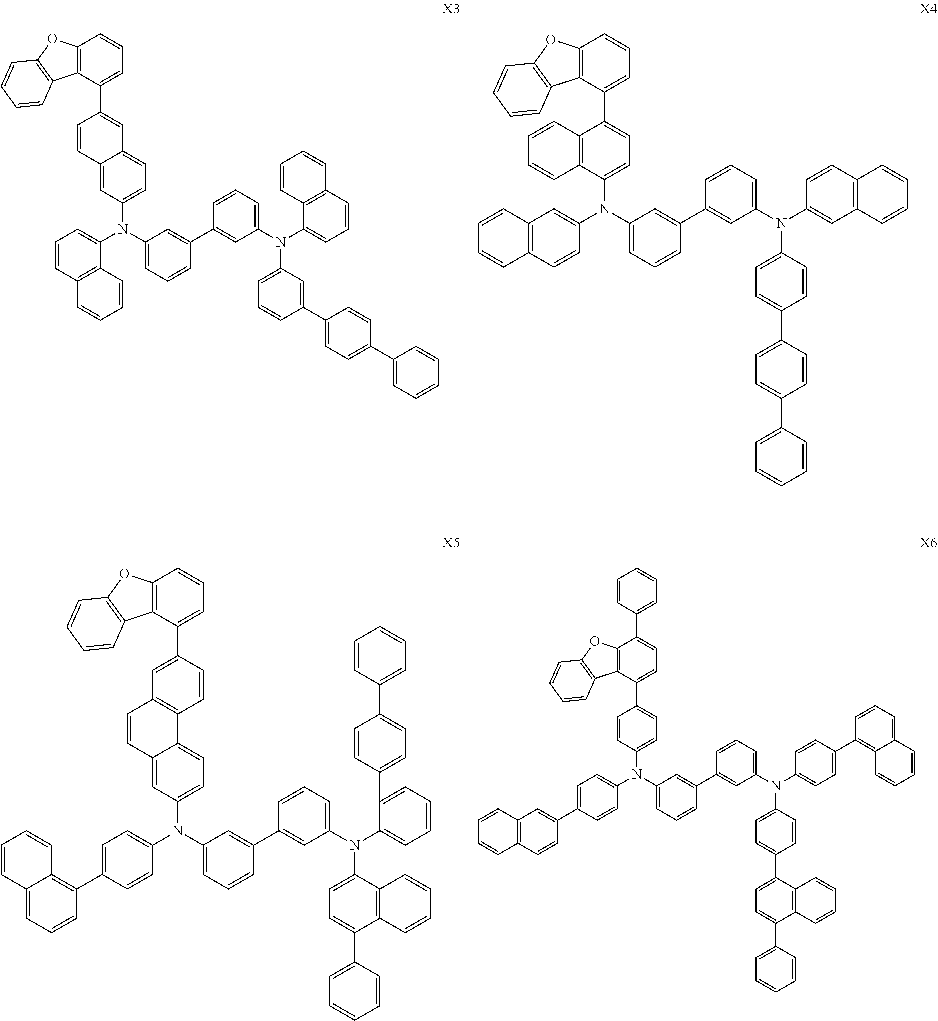 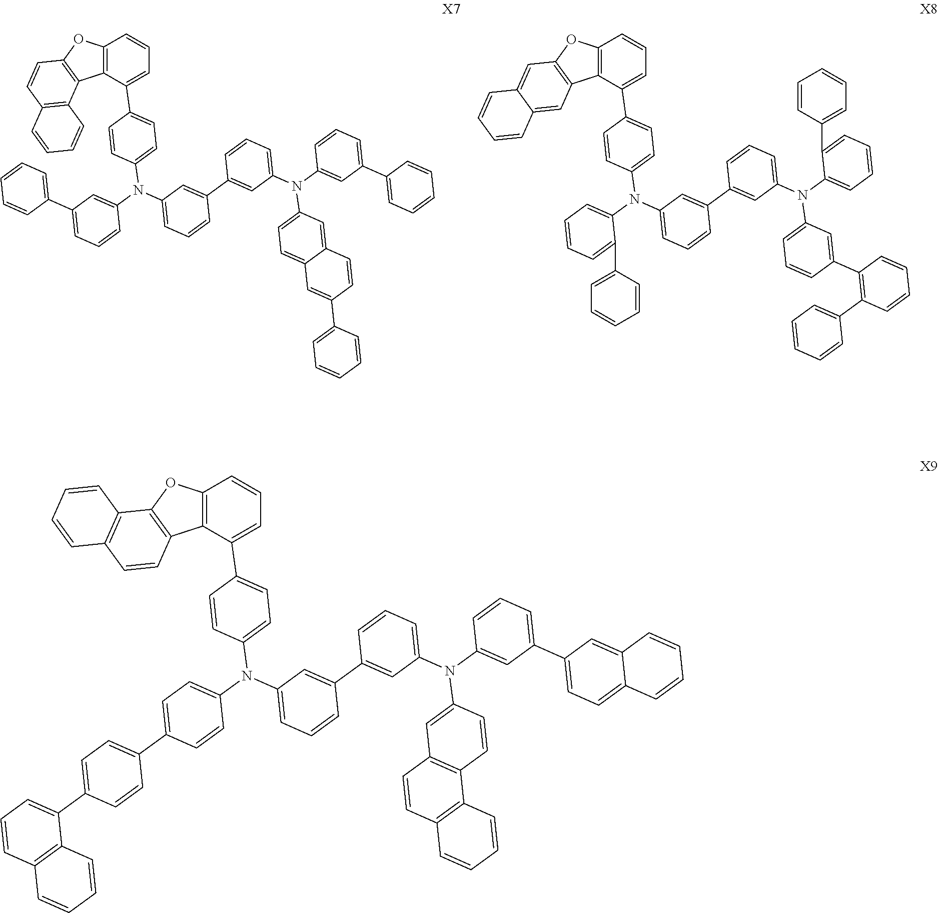 [SMILES: c1ccc(-c2cccc(N(c3ccc(-c4cccc5oc6ccc7ccccc7c6c45)cc3)c3cccc(-c4cccc(N(c5cccc(-c6ccccc6)c5)c5ccc6cc(-c7ccccc7)ccc6c5)c4)c3)c2)cc1 ; c1ccc(-c2ccccc2-c2cccc(N(c3cccc(-c4cccc(N(c5ccc(-c6cccc7oc8cc9ccccc9cc8c67)cc5)c5ccccc5-c5ccccc5)c4)c3)c3ccccc3-c3ccccc3)c2)cc1 ; c1cc(-c2cccc(N(c3cccc(-c4ccc5ccccc5c4)c3)c3ccc4c(ccc5ccccc54)c3)c2)cc(N(c2ccc(-c3ccc(-c4cccc5ccccc45)cc3)cc2)c2ccc(-c3cccc4oc5c6ccccc6ccc5c34)cc2)c1] 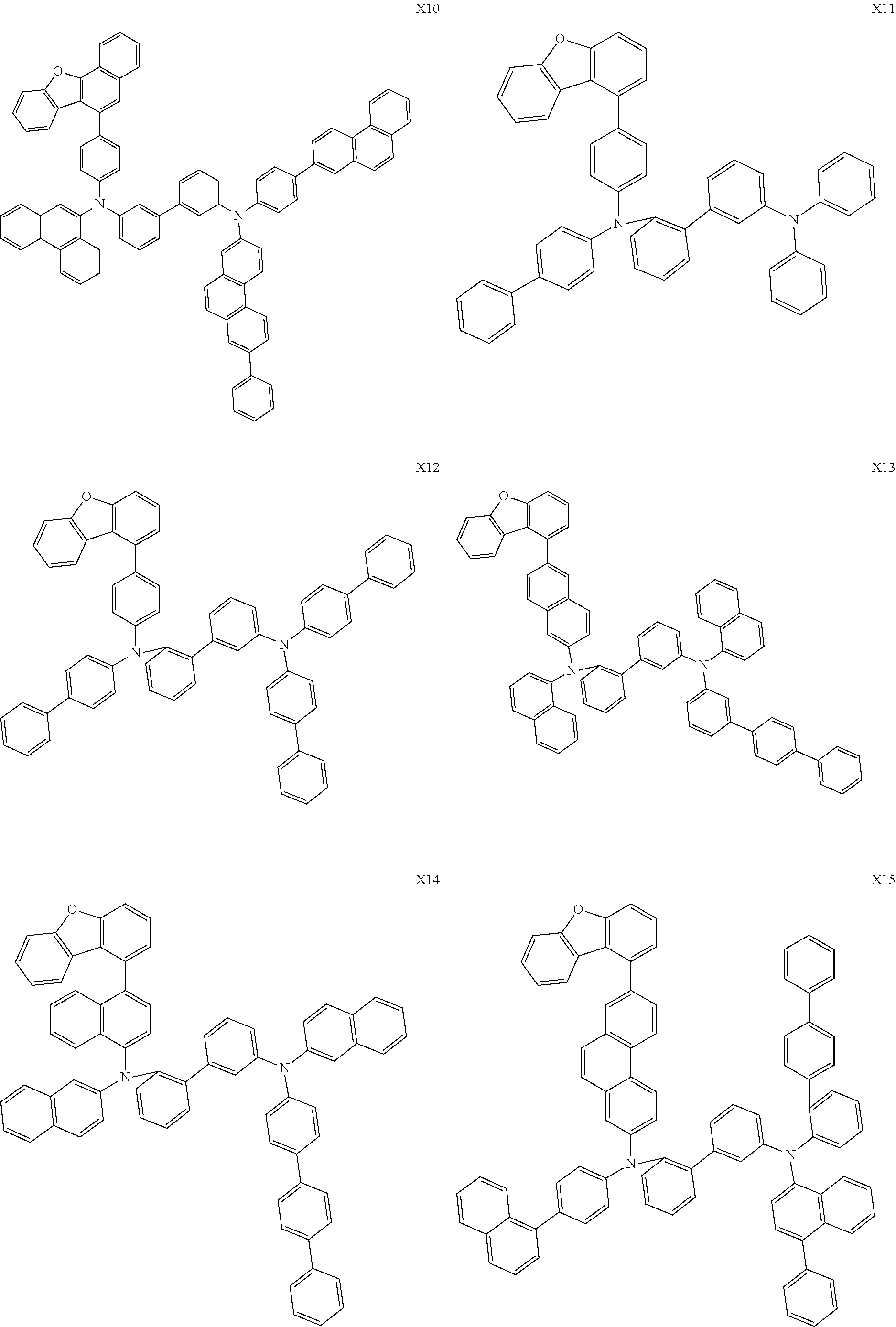 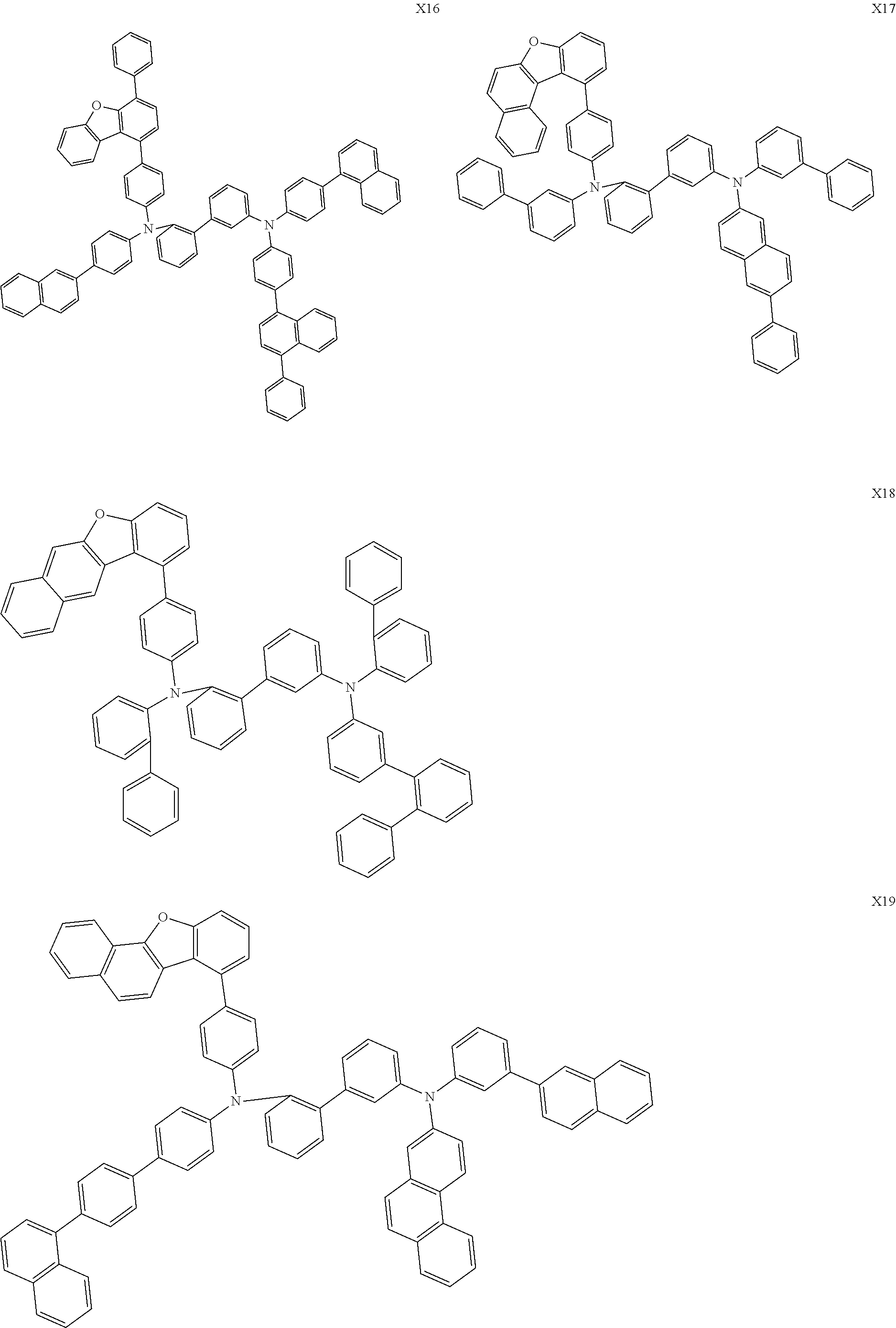 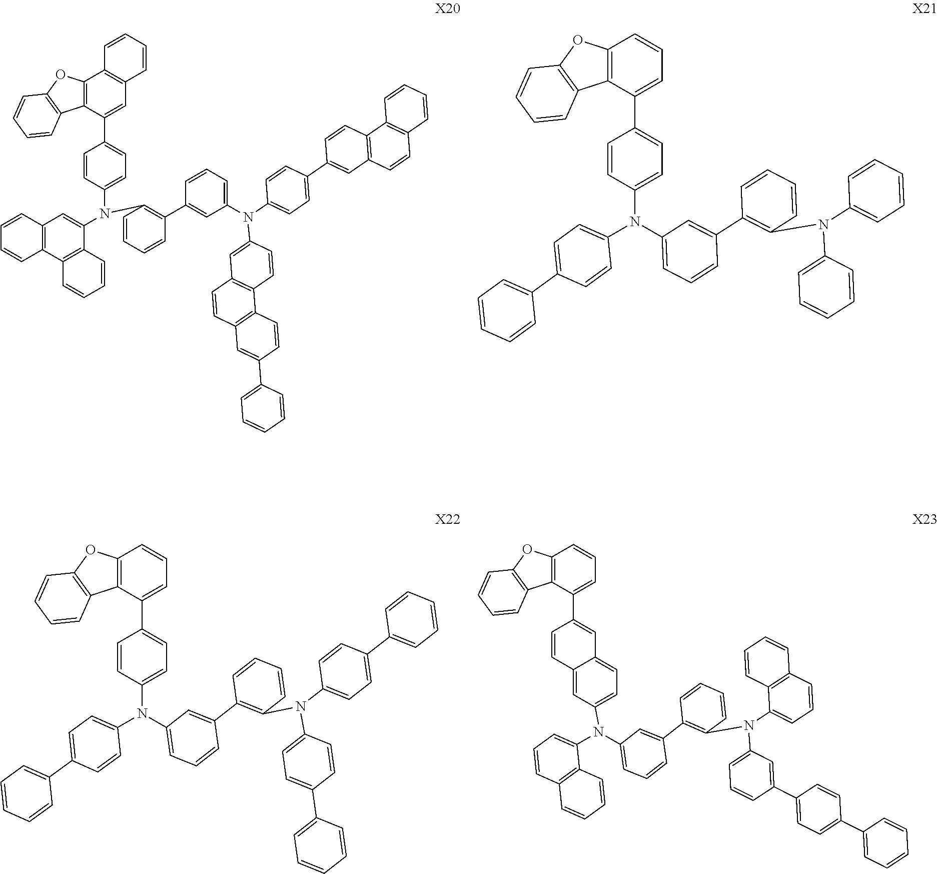 [SMILES: c1ccc(-c2ccc3c(ccc4cc(N(c5ccc(-c6ccc7c(ccc8ccccc87)c6)cc5)c5cccc(-c6ccccc6N(c6ccc(-c7cc8ccccc8c8oc9ccccc9c78)cc6)c6cc7ccccc7c7ccccc67)c5)ccc43)c2)cc1 ; c1ccc(-c2ccc(N(c3ccc(-c4cccc5oc6ccccc6c45)cc3)c3cccc(-c4ccccc4N(c4ccccc4)c4ccccc4)c3)cc2)cc1 ; c1ccc(-c2ccc(N(c3ccc(-c4cccc5oc6ccccc6c45)cc3)c3cccc(-c4ccccc4N(c4ccc(-c5ccccc5)cc4)c4ccc(-c5ccccc5)cc4)c3)cc2)cc1 ; c1ccc(-c2ccc(-c3cccc(N(c4ccccc4-c4cccc(N(c5ccc6cc(-c7cccc8oc9ccccc9c78)ccc6c5)c5cccc6ccccc56)c4)c4cccc5ccccc45)c3)cc2)cc1] 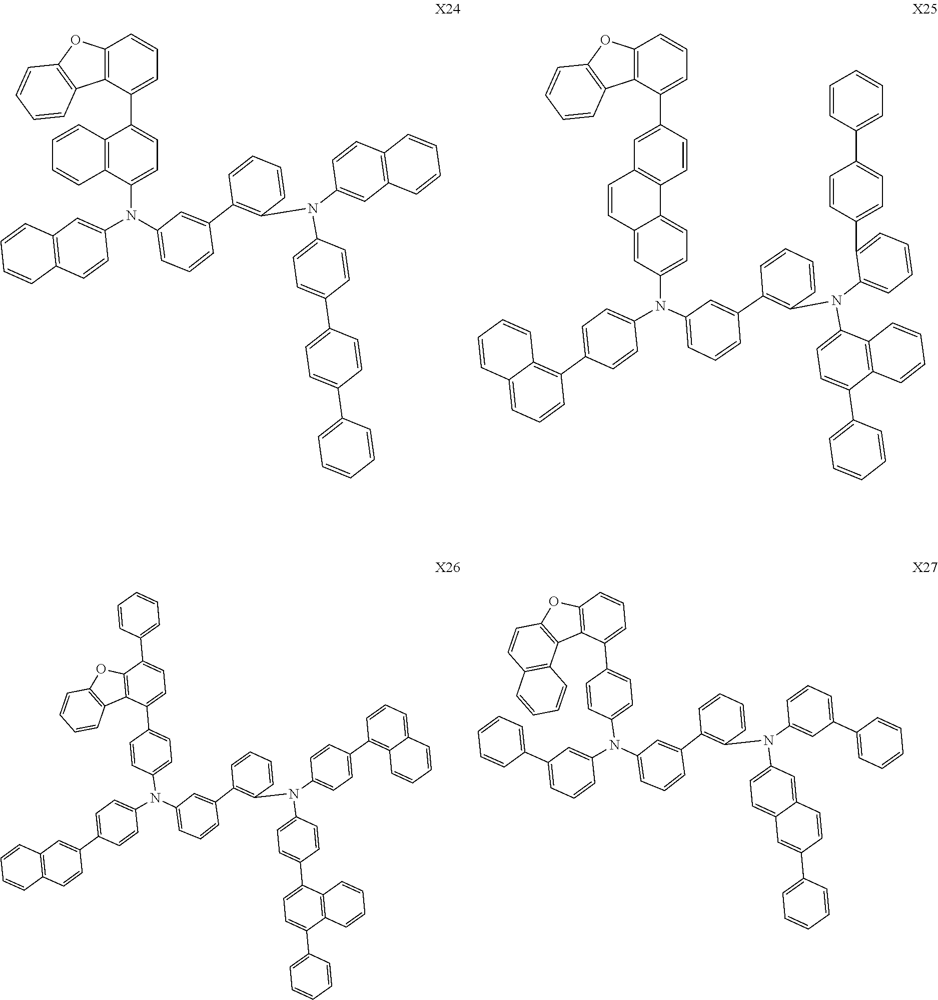 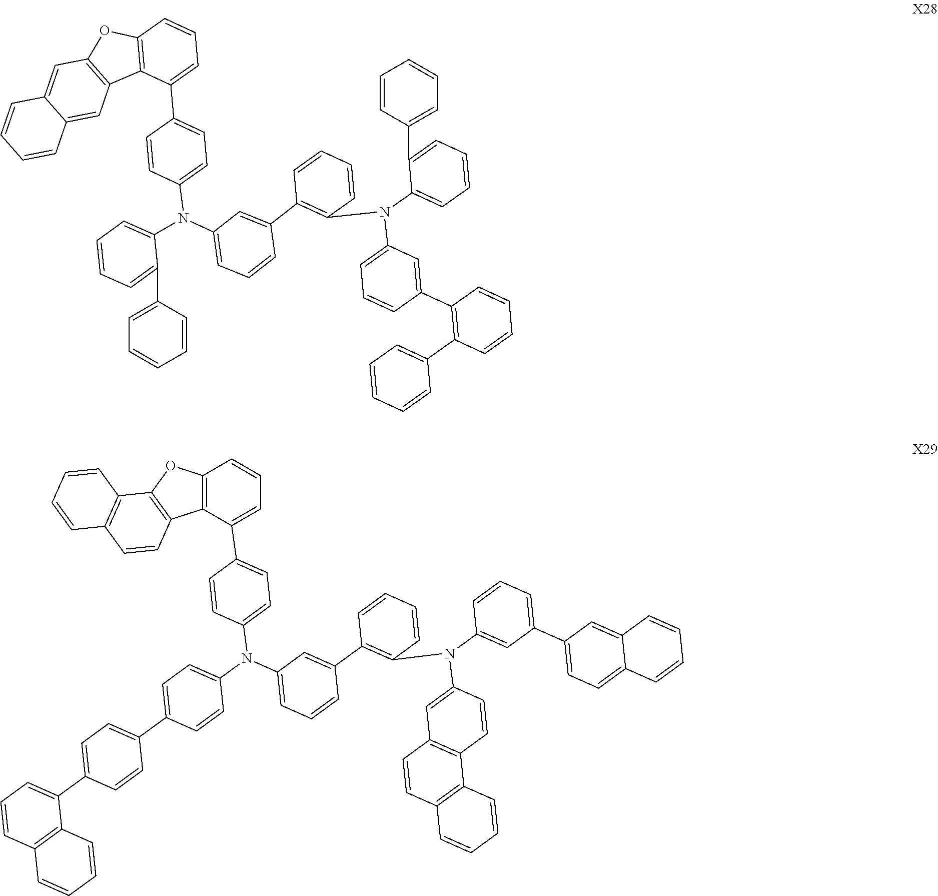 [SMILES: c1ccc(-c2ccccc2-c2cccc(N(c3ccccc3-c3ccccc3)c3ccccc3-c3cccc(N(c4ccc(-c5cccc6oc7cc8ccccc8cc7c56)cc4)c4ccccc4-c4ccccc4)c3)c2)cc1 ; c1cc(-c2ccccc2N(c2cccc(-c3ccc4ccccc4c3)c2)c2ccc3c(ccc4ccccc43)c2)cc(N(c2ccc(-c3ccc(-c4cccc5ccccc45)cc3)cc2)c2ccc(-c3cccc4oc5c6ccccc6ccc5c34)cc2)c1] 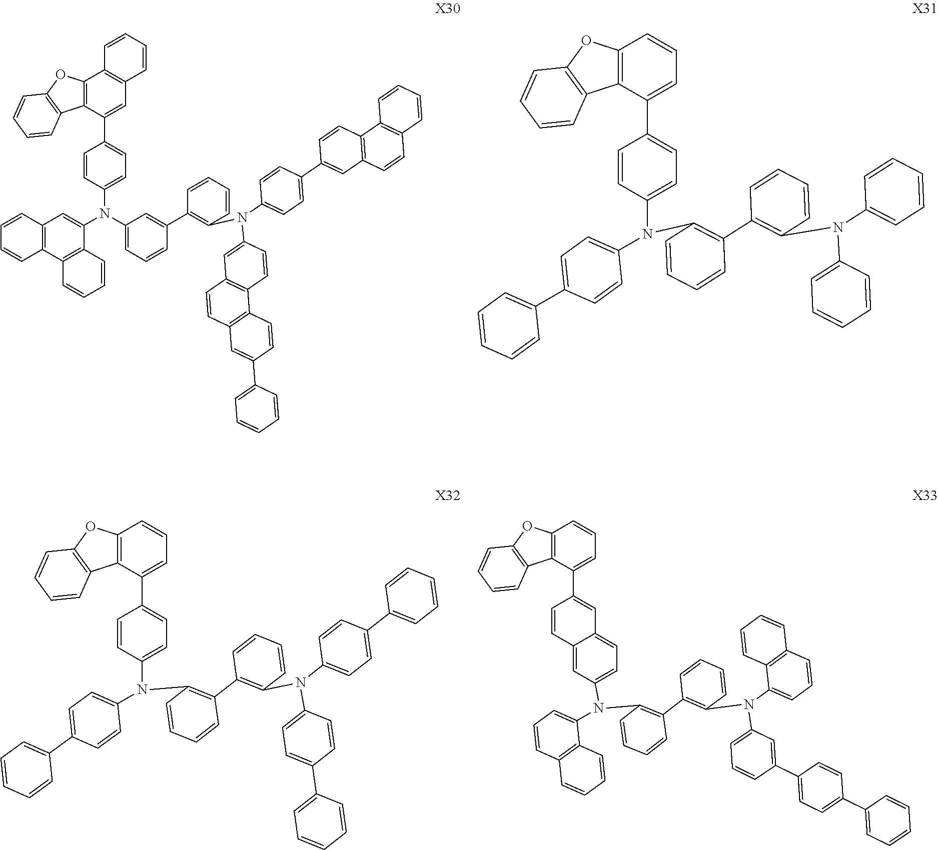 [SMILES: c1ccc(-c2ccc3c(ccc4cc(N(c5ccc(-c6ccc7c(ccc8ccccc87)c6)cc5)c5ccccc5-c5cccc(N(c6ccc(-c7cc8ccccc8c8oc9ccccc9c78)cc6)c6cc7ccccc7c7ccccc67)c5)ccc43)c2)cc1 ; c1ccc(-c2ccc(N(c3ccc(-c4cccc5oc6ccccc6c45)cc3)c3ccccc3-c3ccccc3N(c3ccccc3)c3ccccc3)cc2)cc1 ; c1ccc(-c2ccc(N(c3ccc(-c4ccccc4)cc3)c3ccccc3-c3ccccc3N(c3ccc(-c4ccccc4)cc3)c3ccc(-c4cccc5oc6ccccc6c45)cc3)cc2)cc1 ; c1ccc(-c2ccc(-c3cccc(N(c4ccccc4-c4ccccc4N(c4ccc5cc(-c6cccc7oc8ccccc8c67)ccc5c4)c4cccc5ccccc45)c4cccc5ccccc45)c3)cc2)cc1] 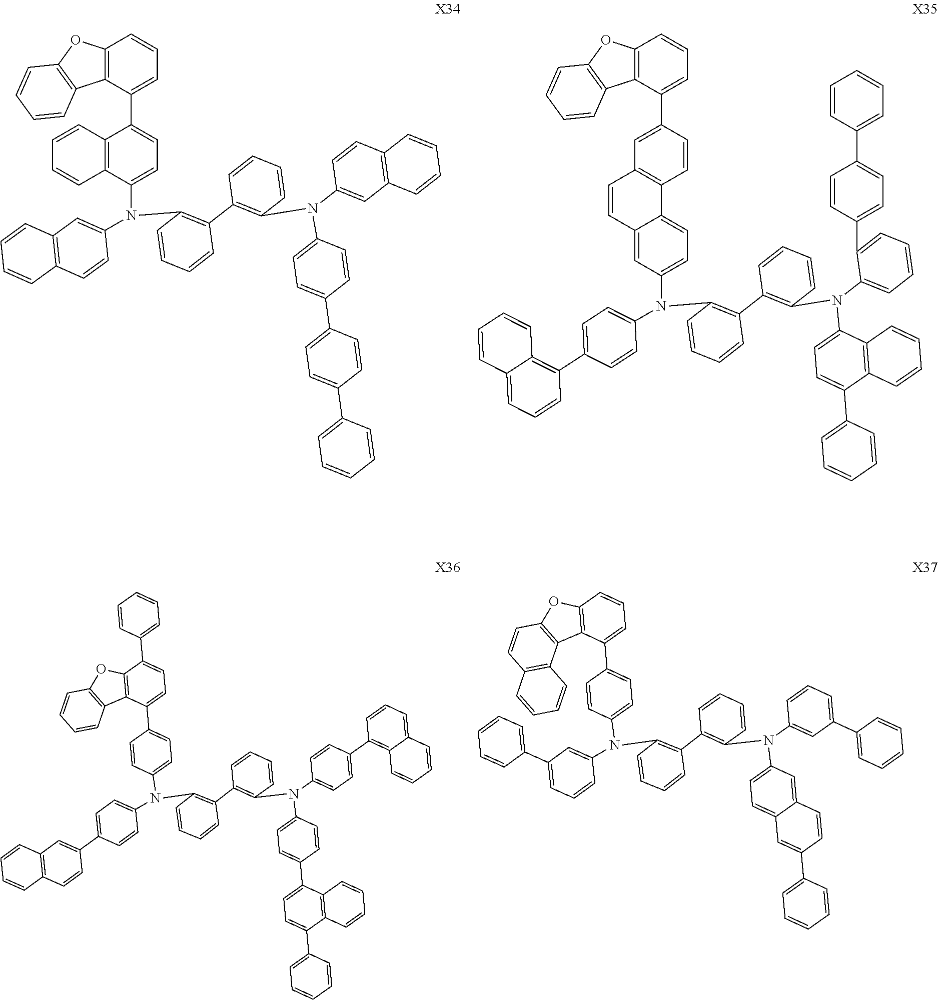 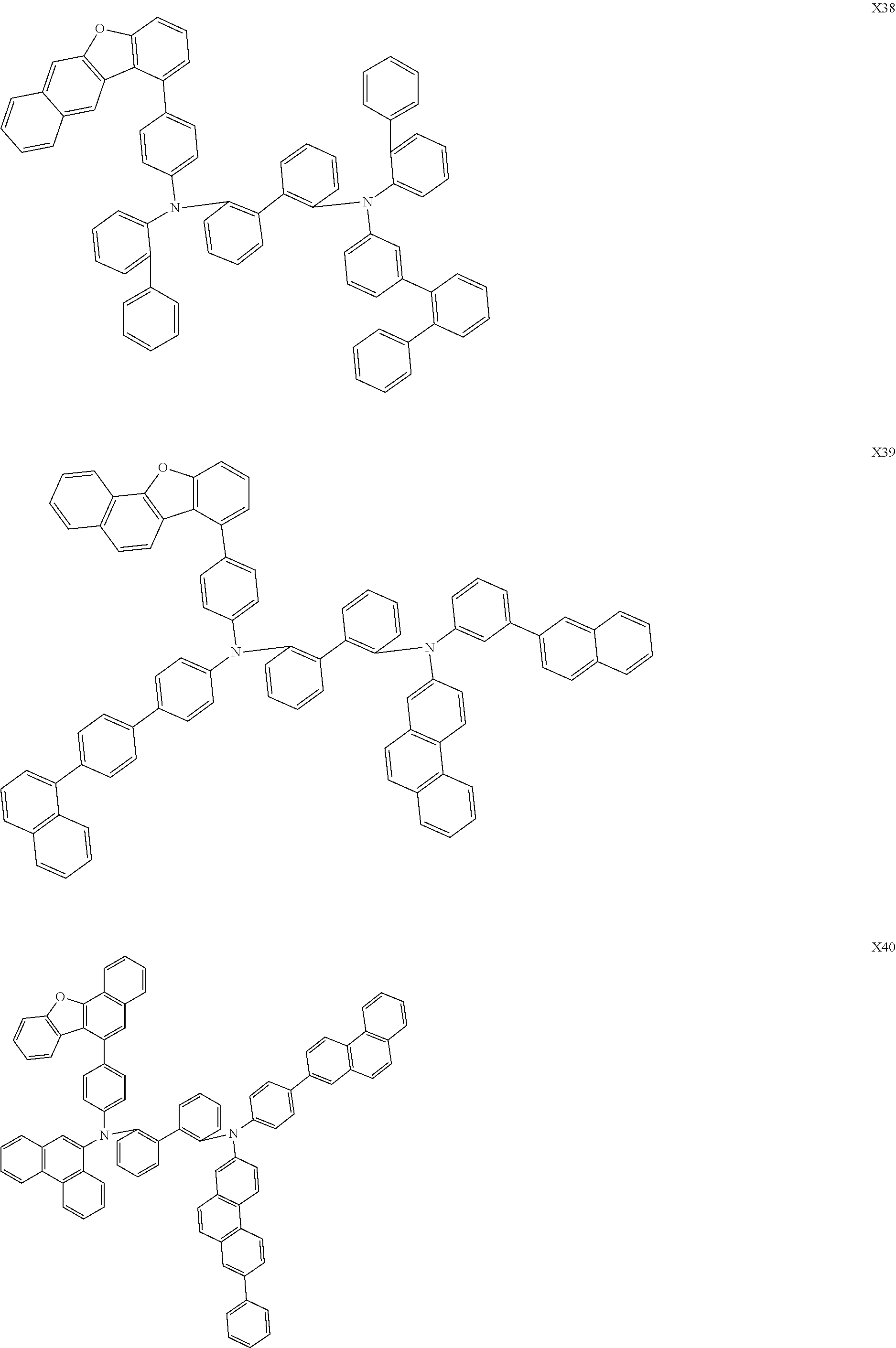 [SMILES: c1ccc(-c2ccccc2-c2cccc(N(c3ccccc3-c3ccccc3)c3ccccc3-c3ccccc3N(c3ccc(-c4cccc5oc6cc7ccccc7cc6c45)cc3)c3ccccc3-c3ccccc3)c2)cc1 ; c1cc(-c2ccc3ccccc3c2)cc(N(c2ccc3c(ccc4ccccc43)c2)c2ccccc2-c2ccccc2N(c2ccc(-c3ccc(-c4cccc5ccccc45)cc3)cc2)c2ccc(-c3cccc4oc5c6ccccc6ccc5c34)cc2)c1 ; c1ccc(-c2ccc3c(ccc4cc(N(c5ccc(-c6ccc7c(ccc8ccccc87)c6)cc5)c5ccccc5-c5ccccc5N(c5ccc(-c6cc7ccccc7c7oc8ccccc8c67)cc5)c5cc6ccccc6c6ccccc56)ccc43)c2)cc1] 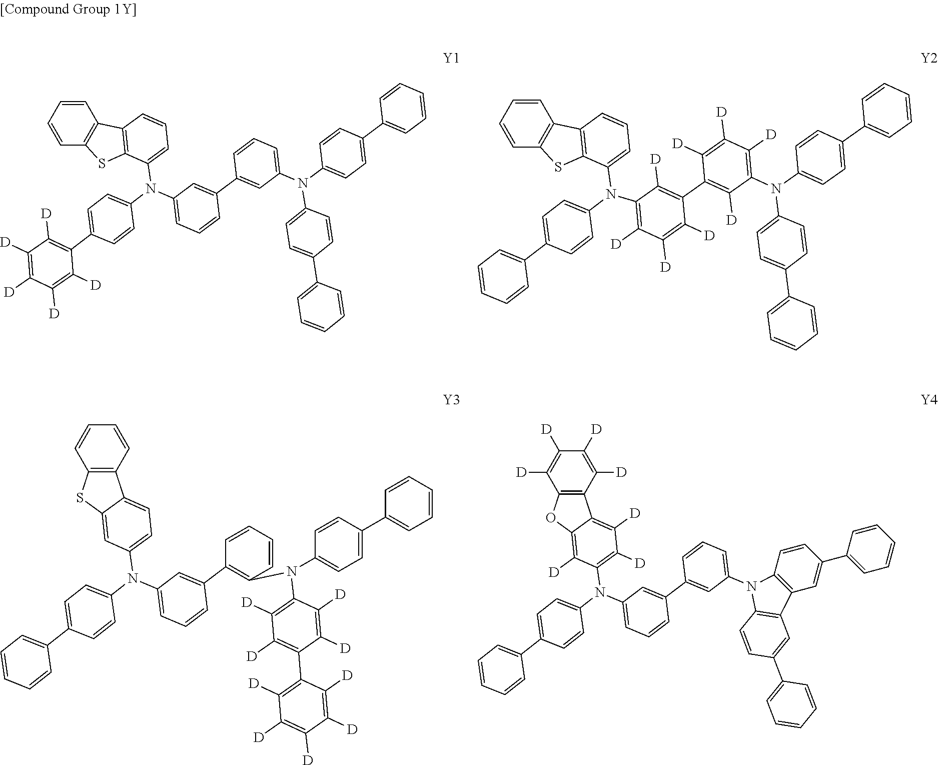 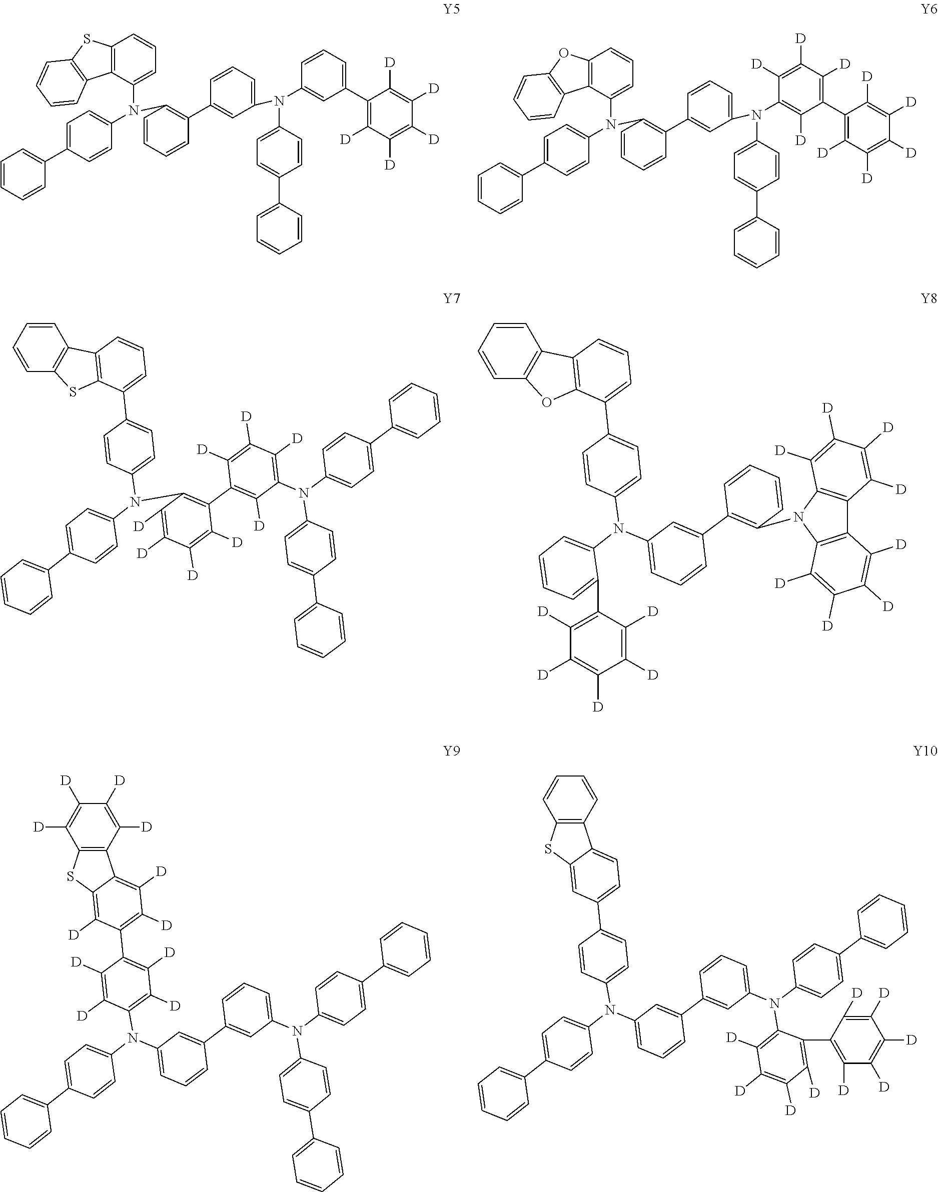 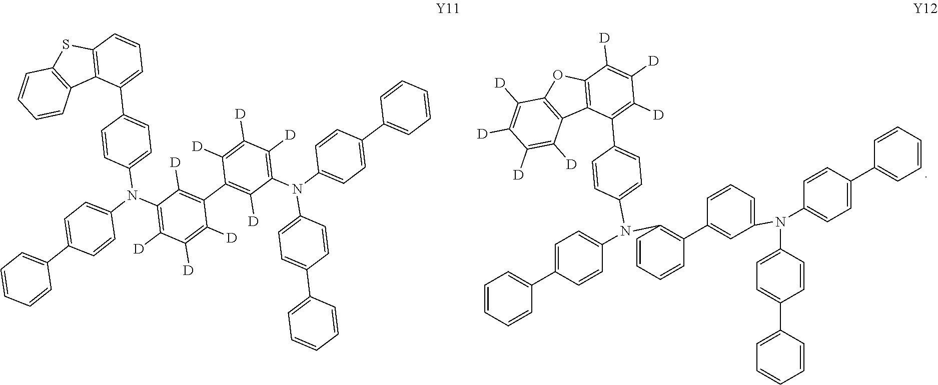 [SMILES: [2H]c1c([2H])c(-c2c([2H])c([2H])c([2H])c(N(c3ccc(-c4ccccc4)cc3)c3ccc(-c4cccc5sc6ccccc6c45)cc3)c2[2H])c([2H])c(N(c2ccc(-c3ccccc3)cc2)c2ccc(-c3ccccc3)cc2)c1[2H] ; [2H]c1c([2H])c([2H])c2c(oc3c([2H])c([2H])c([2H])c(-c4ccc(N(c5ccc(-c6ccccc6)cc5)c5ccccc5-c5cccc(N(c6ccc(-c7ccccc7)cc6)c6ccc(-c7ccccc7)cc6)c5)cc4)c32)c1[2H]]
